(19) United States
(12) Patent Application Publication
(10) Pub. No.: US 2026/0284242 A1
(43) Pub. Date: Sep. 24, 2026

(54) PROTEASE-ACTIVATED RECEPTOR-2 (PAR2) TARGETED THERAPEUTICS AND USES THEREOF

(71) Applicant: Radionetics Oncology, Inc., San Diego, CA (US)

(72) Inventors: Junjie LIU, San Diego, CA (US); Yunfei ZHU, San Diego, CA (US); Yifeng XIONG, San Diego, CA (US)

(21) Appl. No.: 19/554,231

(22) Filed: Mar. 2, 2026

Related U.S. Application Data
(60) Provisional application No. 63/765,904, filed on Mar. 3, 2025.

(51) Int. Cl. A61K 51/08; A61K 51/04; A61P 35/00

(52) U.S. Cl. CPC A61K 51/088; A61K 51/0497; A61P 35/00

(57) ABSTRACT

Described herein are radiotherapeutics that target tumor cells expressing the Protease-Activated Receptor-2 (PAR2) and their use in the treatment and/or diagnosis of cancer.

### CROSS-REFERENCE TO RELATED APPLICATIONS

This application claims the benefit of U.S. Provisional Patent Application No. 63/765,904, filed Mar. 3, 2025, which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 10, 2026, is named 63172-717_201_SL.xml and is 1,433,901 bytes in size.

### FIELD OF THE INVENTION

Described herein are radiotherapeutics that target tumor cells expressing Protease-Activated Receptor-2 (PAR2) and methods of using such radiotherapeutics as cancer therapeutics, diagnostics, or both.

### BACKGROUND OF THE INVENTION

Neoplasms are abnormal growth of cells and cause enormous medical burdens, including morbidity and mortality, in humans. Neoplasms include benign or noncancerous neoplasms which do not display malignant features and are generally unlikely to become dangerous (e.g., adenomas). Malignant neoplasms display features such as genetic mutations, loss of normal function, rapid division, and ability metastasize (invade) to other tissues, and neoplasms of uncertain or unknown behavior. Malignant neoplasms (i.e., cancerous solid tumors) are the leading cause of death in industrialized countries. Noncancerous neoplasms including benign adenomas can also cause significant morbidity and mortality. Although standard treatments can achieve significant effects in tumor growth inhibition and even tumor elimination, the applied drugs exhibit only minor selectivity for the malignant tissue over healthy tissue and their severe side effects limit their efficacy and use. Specific targeting of neoplastic cells without affecting healthy tissue is a major desire for effective solid tumor therapy.

G protein-coupled receptors (GPCRs) are an important class of cell surface receptors that are frequently overexpressed in tumor cells and considered promising targets for selective tumor therapy. PAR2, also known as coagulation factor II (thrombin) receptor-like 1 (F2RL1) or G-Protein coupled receptor 11 (GPR11), is a GPCR overexpressed in several cancers, including, but not limited to, colon cancer, pancreatic cancer, stomach cancer, esophageal cancer, rectal cancer, liver cancer, and lung cancer. As such, targeted delivery of radionuclides to tumors with PAR2-targeting conjugates offers a novel approach to treat and diagnose various cancers.

### SUMMARY OF THE INVENTION

Described herein are radiopharmaceuticals for use in the diagnosis and/or treatment of tumors. The present disclosure provides an alternative and improved method for the treatment of tumors by targeting tumors that overexpress the Protease-Activated Receptor-2 (PAR2). In some embodiments, the radiopharmaceuticals disclosed herein are useful in the treatment of tumors that overexpress PAR2. In some other embodiments, the radiopharmaceuticals disclosed herein are useful in the identification of tissues or organs in a subject comprising tumors overexpressing PAR2. The radiopharmaceuticals disclosed herein are also useful in vivo imaging of a subject for the presence of and distribution of tumors that overexpress PAR2 in the subject.

In one aspect, described herein is a compound of Formula (I), or a pharmaceutically acceptable salt thereof:

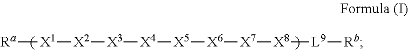

wherein:
R^{a} is —C(═O)—R¹⁰,
R¹⁰ is an aryl, heteroaryl, or partially unsaturated bicyclic heterocyclic ring, each optionally substituted with I to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
L⁹ is an optional linker that is attached to any one of X¹, X², X³, X⁴, X⁵, X⁶, X⁷ or X⁸;
R^{b} is a chelating moiety or a radionuclide complex thereof;
X¹ is

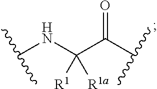

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each Rib is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

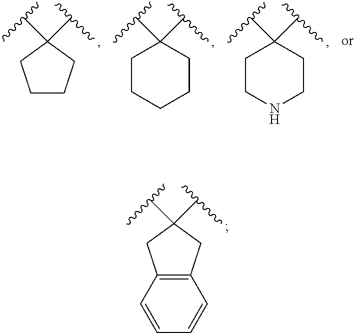

X² is

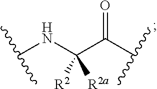

R² is -L²-R^{2b};
L² is absent, —CH₂—, or —CHPh-;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl,
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

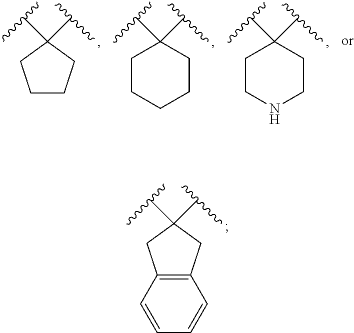

X³ is glycine, (S)-alanine (Ala), (S)-trifluoromethylalanine, (S)-homoalanine, (S)-Glu, (S)-Gln, (S)-Orn, (S)-lysine (Lys), (S)-serine (Ser), (S)—N-methylalanine (N-Me-Ala), 2-amino-2-methylpropanoic acid (AB), I-aminocyclopropane-1-carboxylic acid (ACC), norleucine (Nle), (S)-3-aminoalanine, (S)-2-amino-6-azidohexanoic acid, (S)-2-amino-6-(4-hexyl-1H-1,2,3-triazol-1-yl)hexanoic acid, (S)-2-amino-6-(4-((2-methoxyethoxy)methyl)-1H-1,2,3-triazol-1-yl)hexanoic acid, (S)-2-amino-6-(4-(cyclopentylmethyl)-1H-1,2,3-triazol-1-yl)hexanoic acid, (S)-2-amino-6-(4-benzyl-1H-1,2,3-triazol-1-yl)hexanoic acid, (S)-proline (Pro), (S)-cyclopropylglycine, or (S)-biphenylalanine (Bip);
X⁴ is (S)-arginine (Arg), (S)-3-amino-6-guanidinohexanoic acid, N6-carbaamimidoyl-(S)-lysine, (S)-2-amino-5-ureidopentanoic acid, (S)-homoarginine, N2-methyl-(S)-arginine, N^{ω}-methyl-(S)-arginine, N^{ω}-nitro-(S)-arginine, (E)-N^{ω},N^{ω}′-dimethyl-(S)-arginine, (E)-N^{ω},N^{ω}′-diethyl-(S)-arginine, (S)-ornithine (Orn), (S)-lysine, (S)-phenylalanine (Phe), (S)-cyclohexylglycine (Chg), (S)-cyclohexylalanine (Cha), norleucine (Nle), (S)-3-amino-alanine (H-Dap-OH), piperidinyl-1-yl alanine, N⁴,N⁴-dimethyl-(S)-asparagine (N⁴,N⁴-dimethyl-Asn), (S)-2-(piperidin-4-yl)-glycine (Gly(piperidin-4-yl)), (S)-3-pyridin-3-yl-beta-alanine (H-βAla(3-pyrid-3-yl)-OH), (S)-2-(1-carbamimidoylpiperidin-4-yl)-glycine (Gly(1-carbamimidoylpiperidin-4-yl)), (S)-3-amino-3-(1-carbamimidoylpiperidin-3-yl)propanoic acid, (S)-phenylglycine (Phg), (S)-4-fluorophenylalanine (Phe(4-F)), (S)-4-aminophenylalanine (Phe(4-NH₂)), (S)-4-nitrophenylalanine (Phe(4-NO₂)), (S)-4-carbamoylphenylalanine, (S)-4-guanidinophenylalanine, (S)-asparagine (Asn), (S)-glutamic acid (Glu), (S)-proline, (S)-4-ureidophenylalanine, (S)-(2-aminoethoxy)phenylalanine (Phe(4-OCH₂CH₂NH₂)), (S)-(pyridin-4-yl)alanine (3-Pal), (S)-(pyridin-4-yl)alanine (4-Pal), (S)-3-(6-aminopyridin-3-yl)alanine ((3-Pal(6-NH₂)), (S)-2-((2-aminopyridin-4-yl)-λ²-azaneyl)ethyl alanine, N6-nicotinoyl-Lys, (S)-2-oxo-2-(piperazin-1-yl)ethyl alanine, (S)-2-(piperidin-4-yl)ethyl alanine, (S)-3-((piperazin-1-yl)propyl)-glycine, (S)-3-((4-methylpiperazin-1-yl)propyl)-glycine, (S)-3-(4-benzylpiperidin-1-yl)propylglycine, (S)-3-((4-benzylpiperazin-1-yl)propyl)-glycine, N6-(1-benzylpiperidine-4-carbonyl)-Lys, (S)-3-((3,4-dihydroisoquinolin-2(1H)-yl)propyl)-glycine, (S)-2-pyridin-4-ylthynyl alanine, (S)-(4-(aminomethyl)-1H-1,2λ²,3λ²-triazol-1-yl)methyl)-glycine, (S)-(4-(piperazin-1-ylmethyl)-1H-1,2λ²,3λ²-triazol-1-yl)methyl)-glycine, (S)-2-((1H-imidazol-4-yl)ethyl)glycine, or (4-((R)-2,6-dioxohexahydropyrimidine-4-carboxamido)phenyl)alanine;
or X⁴ is (S)-4-methylpiperazine-2-carboxylic acid,
or X⁴ is

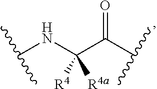

where R⁴ and R^{4a} are taken together with the carbon atom to which they are attached to form

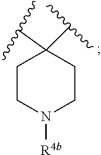

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

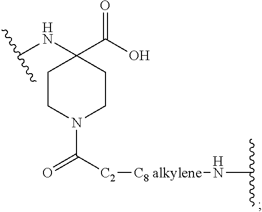

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid

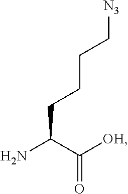 [SMILES: [N-]=[N+]=NCCCC[C@H](N)C(=O)O]

N6-acetyl-lysine

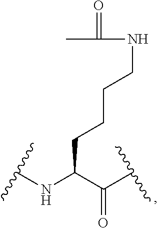 [SMILES: *N[C@@H](CCCCNC(C)=O)C(*)=O ; ","]

N6-(pent-4-ynoyl)-lysine

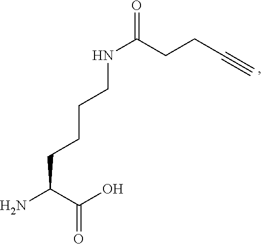 [SMILES: C#CCCC(=O)NCCCC[C@H](N)C(=O)O ; ","]

N6-octanoyl-lysine

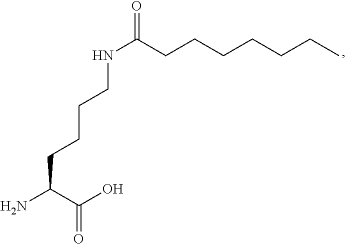 [SMILES: CCCCCCCC(=O)NCCCC[C@H](N)C(=O)O ; ","]

tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-I)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (S)-(4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

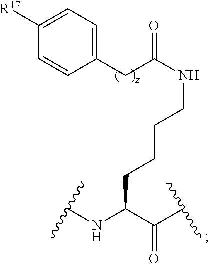

R¹⁷ is —CH₃ or —I;
z is 1, 2, or 3;
X⁸ is absent, lysine (Lys), or glutamic acid (Glu);
L⁵, L⁶, and L⁷ are each independently —NR¹⁵-L¹³-L¹⁴-C(═O)— or substituted or unsubstituted piperazinylene-;
L¹³ is absent, —C₁-C₆ alkylene, substituted or unsubstituted heterocycloalkylene, substituted or unsubstituted cyclohexylene, —(CH₂CH₂O)_{y}—, or —(CH₂CH₂NR¹⁶)_{y}—;
L¹⁴ is absent, —C₁-C₆ alkylene, substituted or unsubstituted heterocycloalkylene, or substituted or unsubstituted cyclohexylene;
R¹⁵ and R¹⁶ are each independently selected from H or —C₁-C₄ alkyl; and
y is 1,2, 3, 4, 5, or 6;
wherein the compound of Formula (I) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (I) may be either —COOH or —CONH₂.

In some embodiments,
R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
L⁹ is an optional linker that is attached to any one of X⁵, X⁶, or X⁷;
R^{b} is a chelating moiety or a radionuclide complex thereof;
X¹ is

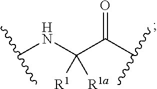

R¹ is -L¹-R^{1b}; L¹ is absent or —CH₂—; Rib is cyclopentyl, cyclohexyl, cycloheptyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, 2-chlorophenyl, 3-chlorophenyl, 4-chlorophenyl, 2,4-dichlorophenyl, 3,4-dichlorophenyl, or 2,5-dichlorophenyl;
R^{1a} is hydrogen or methyl;
X² is

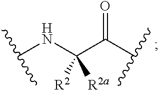

R² is -L²-R^{2b}; L² is absent or —CH₂—; R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl, or 4′-methyl-[1,1′-biphenyl]-4-yl;
R^{2a} is hydrogen or methyl;
X³ is glycine, (S)-alanine (Ala), or norleucine (Nle);
X⁴ is (S)-arginine (Arg), (S)-ornithine (Om), (S)-phenylalanine (Phe), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-(pyridin-4-yl)alanine (4-Pal), (S)-3-(6-aminopyridin-3-yl)alanine ((3-Pal(6-NH₂)), or (S)-2-((2-aminopyridin-4-yl)-λ²-azaneyl)ethyl alanine;
X⁵ is alanine (Ala), lysine (Lys), ornithine (Orn),

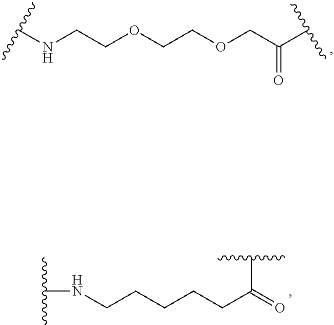 [SMILES: *NCCOCCOCC(*)=O ; *NCCCCCC(*)=O]

N6-(4-trifluorobenzoyl)-Lys, β³-homolysine, 3-amino-alanine (H-Dap-OH), 4-nitrophenylalanine (Phe(4-NO₂)), or (4-fluorobenzamido)methylglycine;
X⁶ is absent, lysine (Lys), ornithine (Orn),

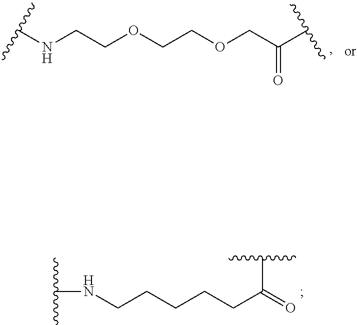 [SMILES: *NCCOCCOCC(*)=O ; *NCCCCCC(*)=O]

X⁷ is absent, lysine (Lys), ornithine (Orn), or

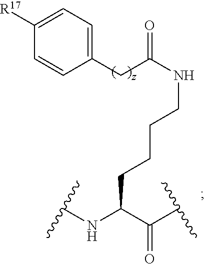

R¹⁷ is —CH₃ or —I;
z is 1, 2, or 3; and
X⁸ is absent;
wherein the compound of Formula (I) is optionally substituted with —C(═O)—C₁-C₁₄ alkylene-R¹², wherein R¹² is H, —CH₃, —COOH, —OH, —NH, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (I) may be either —COOH or —CONH₂.

In some embodiments, R^{a} is —C(═O)—R¹⁰, wherein R¹⁰ is a monocyclic heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl; and wherein R¹⁰ is optionally substituted with 1 to 3 substituents that are each independently selected from the group consisting of halo, C₁-C₆ alkyl, alkoxy, —OH, —NH₂, —NH—C₁-C₆ alkylene-R¹¹, and —O—C₁-C₆ alkylene-R¹¹.

In some embodiments, R^{b} is a chelating moiety independently selected from the group consisting of:
1,4,7,10-tetraazacyclododecane-1,4,7,10-tetraacetic acid (DOTA);
2,2′,2″-(10-(2-amino-2-oxoethyl)-1,4,7,10-tetraazacyclododecane-1,4,7-triyl)triacetic acid (PSC);
1,4,7,10-tetraazacyclododecane-1,4,7-triacetic acid (DO3A);
1,4,7,10-tetraazacyclododecane-1,7-diacetic acid (DO2A);
α,α′,α″,α′″-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid;
benzyl-1,4,7,10-tetraazacyclododecane-1,4,7,10-tetraacetic acid (Bn-DOTA);
p-hydroxy-benzyl-1,4,7,10-tetraazacyclododecane-1,4,7,10-tetraacetic acid (p-OH-Bn-DOTA);
6,6′-(((pyridine-2,6-diylbis(methylene))bis((carboxymethyl)azanediyl))bis(methylene))dipicolinic acid (H₄pypa);
H₄pypa-benzyl;
6,6′,6″,6′″-(((pyridine-2,6-diylbis(methylene))bis(azanetriyl))tetrakis(methylene))-tetrapicolinic acid (H₄py4pa);
H₄py4pa-benzyl;
2,2′,2″-(1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2′,2″,2″′-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H₄octapa-benzyl; and
3,6,9,12-tetrakis(carboxymethyl)-3,6,9,12-tetraazatetradecanedioic acid (TTHA); or a radionuclide complex thereof

In some embodiments, R^{b} is a chelating moiety independently selected from the group consisting of:

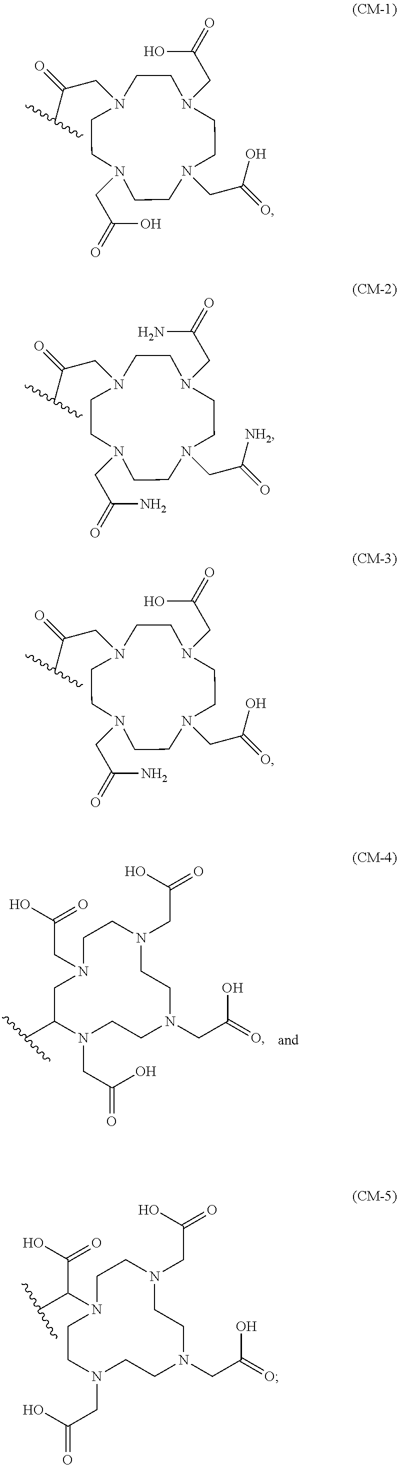 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; "and" ; *C(C(=O)O)N1CCN(CC(=O)O)CCN(CC(=O)O)CCN(CC(=O)O)CC1]

or a radionuclide complex thereof.

In some embodiments, L⁹ is: —C(═O)-L¹⁰-L¹¹-L¹² or —CH₂—C(═O)-L¹⁰-L¹¹-L¹²-;
L¹⁰ is absent, —C₁-C₆ alkylene, —NR¹³—, substituted or unsubstituted heterocycloalkylene, or substituted or unsubstituted cyclohexylene;
L¹¹ is absent, —C₁-C₆ alkylene, substituted or unsubstituted heterocycloalkylene, substituted or unsubstituted cyclohexylene, or —(OCH₂CH₂)ₓ—;
L¹² is —NR¹⁴—;
R¹³ and R¹⁴ are each independently selected from H or —C₁-C₄ alkyl; and
x is 1, 2, 3, 4, 5, or 6.

In some embodiments, the radionuclide of the radionuclide complex is: an Auger electron-emitting radionuclide; or an α-emitting radionuclide; or a β-emitting radionuclide; or a γ-emitting radionuclide.

In some embodiments, the radionuclide of the radionuclide complex is a lanthanide or an actinide.

In some embodiments, the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

In some embodiments, the radionuclide of the radionuclide complex is a diagnostic or therapeutic radionuclide.

In some embodiments, the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 71-gallium (71Ga), 99m-technetium (^{99m}Tc), 195m-platinum (^{195m}Pt), 225-actinium (²²⁵Ac), 213-bismuth (²¹³Bi), 223-radium (²²³Ra), 211-astatine (²¹¹At), aluminium-[¹⁸F]fluoride ([¹⁸F]AlF), 90-yttrium (⁹⁰Y), 175-lutetium (¹⁷⁵Lu), 177-lutetium (¹⁷⁷Lu), 161-terbium (¹⁶¹Tb), 186-rhenium (¹⁸⁶Re), 188-rhenium (¹⁸⁸Re), 204-lead (²⁰⁴Pb), 206-lead (²¹⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), 153-samarium (¹⁵³Sm), 89-strontium (⁸⁹Sr), 198-gold (¹⁹⁸Au), 169-erbium (¹⁶⁹Er), 165-dysprosium (¹⁶⁵Dy), 99m-technetium (^{99m}Tc), 89-zirconium (⁸⁹Zr), 52-manganese (⁵²Mn), 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra). In some embodiments, the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 211-astatine (²¹¹At), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (²⁰⁷Pb), 208-lead (²⁰⁸Pb) 212-lead (212Pb), 63-copper (⁶³Cu), 64-copper (⁶⁴Cu), 65-copper (⁶⁵Cu), or 67-copper (⁶⁷Cu).

In some embodiments, the radionuclide of the radionuclide complex is:
an Auger electron-emitting radionuclide that is 111-indium (¹¹¹In), 67-gallium (⁶⁷Ga), 68-gallium (⁶⁸Ga), 99m-technetium (⁹⁹-Tc), or 195m-platinum (^{195m}Pt); or
an α-emitting radionuclide that is 225-actinium (²²⁵Ac), 213-bismuth (²¹³Bi), 223-radium (²²³Ra), or 212-lead (²¹²Pb); or
a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or
a γ-emitting radionuclide that is 60-cobalt (⁶¹Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is aluminium-[¹⁸F]fluoride ([¹⁸F]AlF), 111-indium (¹¹¹In), 115-indium (¹¹⁵In), 90-yttrium (⁹⁰Y), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), or 213-bismuth (²¹³Bi).

In some embodiments, Z′ is 90-yttrium (⁹⁰Y), 67-gallium (⁶⁷Ga), 68-gallium (⁶⁸Ga), 225-actinium (²²⁵Ac), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (⁷⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), or 213-bismuth (²¹³Bi).

In some embodiments, Z′ is 225-actinium (²²⁵Ac), 177-lutetium (¹⁷⁷Lu), 161-terbium (¹⁶¹Tb), 204-lead (²⁰⁴Pb), 212-lead (²¹²Pb), 67-copper (⁶⁷Cu), 90-yttrium (⁹⁰Y), or 213-bismuth (²¹³Bi).

Also described herein is a pharmaceutical composition comprising a compound described herein (e.g., a compound of Formula (I), or a pharmaceutically acceptable salt thereof, and at least one pharmaceutically acceptable excipient. In some embodiments, the pharmaceutical composition is formulated for administration to a mammal by intravenous administration or subcutaneous administration. In some embodiments, the pharmaceutical composition is formulated for administration to a mammal by intravenous administration.

In another aspect, described herein is a method for the treatment of cancer comprising administering to a mammal with cancer an effective amount of a compound described herein (e.g., a compound of Formula (I)), or a pharmaceutically acceptable salt thereof, or an effective amount of pharmaceutical composition comprising a compound described herein (e.g., a compound of Formula (I)), or a pharmaceutically acceptable salt thereof. In some embodiments, the cancer comprises tumors and the tumors overexpress the Protease-Activated Receptor-2 (PAR2). In some embodiments, the cancer is colon cancer, pancreatic cancer, stomach cancer, esophageal cancer, rectal cancer, liver cancer, or lung cancer. In some embodiments, the cancer is colon cancer. In some embodiments, the cancer is pancreatic cancer. In some embodiments, the cancer is stomach cancer. In some embodiments, the cancer is esophageal cancer. In some embodiments, the cancer is rectal cancer. In some embodiments, the cancer is liver cancer. In some embodiments, the cancer is lung cancer.

In another aspect, described herein is a method for treating tumors in a mammal with a radionuclide comprising administering to the mammal a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof. In some embodiments, the mammal has been diagnosed with breast cancer. In some embodiments, the mammal has been diagnosed with renal cancer. In some embodiments, the mammal has been diagnosed with lung cancer.

In another aspect, described herein is a method of targeting delivery of a radionuclide to tumors in a mammal comprising administering to a mammal with tumors a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof; wherein the tumors overexpress the Protease-Activated Receptor-2 (PAR2).

In another aspect, described herein is a method for identifying tumors expressing the Protease-Activated Receptor-2 (PAR2) in a mammal comprising administering to the mammal a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof; and performing positron emission tomography (PET) analysis, single-photon emission computerized tomography (SPECT), or magnetic resonance imaging (MRI); wherein R^{b} is a chelating moiety-diagnostic radionuclide complex. In another aspect, described herein is a method for identifying tumors expressing the Protease-Activated Receptor-2 (PAR2) in a mammal comprising administering to the mammal a compound described herein (e.g., a compound of Formula (I)), or a pharmaceutically acceptable salt thereof, or a pharmaceutical composition comprising a compound described herein (e.g., a compound of Formula (1)), or a pharmaceutically acceptable salt thereof; and performing positron emission tomography (PET) analysis or single-photon emission computerized tomography (SPECT); wherein R^{b} is a chelating moiety-diagnostic radionuclide complex.

In yet another aspect, described herein is a method for the in vivo imaging of tissues or organs in a mammal with tumors expressing the Protease-Activated Receptor-2 (PAR2) comprising administering to the mammal a compound described herein (e.g., a compound of Formula (1)), or a pharmaceutically acceptable salt thereof, or a pharmaceutical composition comprising a compound described herein (e.g., a compound of Formula (I)), or a pharmaceutically acceptable salt thereof; and performing positron emission tomography (PET) analysis, single-photon emission computerized tomography (SPECT), or magnetic resonance imaging (MRI); wherein R^{b} is a chelating moiety-diagnostic radionuclide complex. In yet another aspect, described herein is a method for the in vivo imaging of tissues or organs in a mammal with tumors expressing the Protease-Activated Receptor-2 (PAR2) comprising administering to the mammal a compound described herein (e.g., a compound of Formula (I)), or a pharmaceutically acceptable salt thereof, or a pharmaceutical composition comprising a compound described herein (e.g., a compound of Formula (I)), or a pharmaceutically acceptable salt thereof; and performing positron emission tomography (PET) analysis or single-photon emission computerized tomography (SPECT); wherein R^{b} is a chelating moiety-diagnostic radionuclide complex.

In one aspect, described herein is a PAR2 ligand having the structure of Formula (VII), or a pharmaceutically acceptable salt thereof.

In some embodiments, described herein is a compound of Formula (VII), or a pharmaceutically acceptable salt thereof:

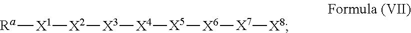

wherein:
R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
X¹ is

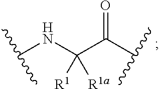

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

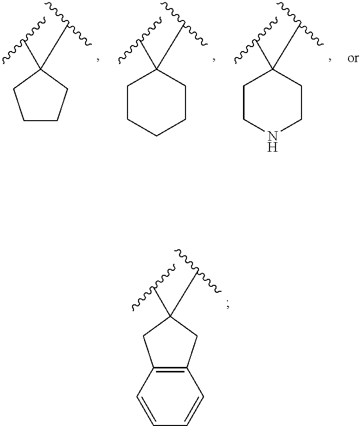 [SMILES: *C1(*)CCCC1 ; *C1(*)CCCCC1 ; *C1(*)CCNCC1 ; *C1(*)Cc2ccccc2C1]

X² is

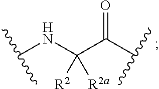

R² is -L²-R^{2b};
L² is absent, —CH₂—, or —CHPh-;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

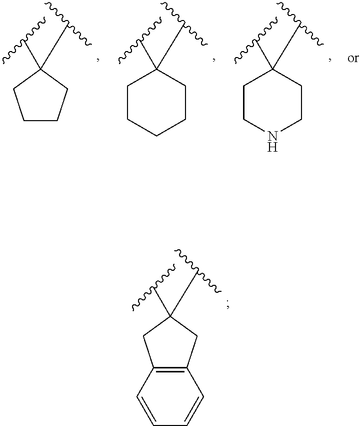 [SMILES: *C1(*)CCCC1 ; *C1(*)CCCCC1 ; *C1(*)CCNCC1 ; *C1(*)Cc2ccccc2C1]

X³ is glycine, alanine (Ala), trifluoromethylalanine, homoalanine, glutamic acid (Glu), glutamine (Gin), ornithine (Orn), lysine (Lys), serine (Ser), N-methylalanine (N-Me-Ala), 2-amino-2-methylpropanoic acid (AIB), 1-aminocyclopropane-1-carboxylic acid (ACC), norleucine (Nle), 3-aminoalanine, 2-amino-6-azidohexanoic acid, 2-amino-6-(4-hexyl-1H-1,2,3-triazol-1-yl)hexanoic acid, 2-amino-6-(4-((2-methoxyethoxy)methyl)-1H-1,2,3-triazol-l-yl)hexanoic acid, 2-amino-6-(4-(cyclopentylmethyl)-1H-1,2,3-triazol-1-yl)hexanoic acid, 2-amino-6-(4-benzyl-1H-1,2,3-triazol-1-yl)hexanoic acid, proline (Pro), cyclopropylglycine, or biphenylalanine (Bip);
X⁴ is arginine (Arg), 3-amino-6-guanidinohexanoic acid, N6-carbamimidoyl-lysine, 2-amino-5-ureidopentanoic acid, homoarginine, N-methyl-arginine, N^{ω}-methyl-arginine, N^{ω}-nitro-arginine, (E)-N^{ω},N^{ω}′-dimethyl-arginine, (E)-N^{ω},N^{ω}′-diethyl-arginine, ornithine (Orn), lysine, phenylalanine (Phe), cyclohexylglycine (Chg), cyclohexylalanine (Cha), norleucine (Nle), 3-amino-alanine (H-Dap-OH), piperidinyl-1-alanine, N⁴,N⁴-dimethyl-asparagine (N⁴,N⁴-dimethyl-Asn), 2-(piperidin-4-yl)-glycine (Gly(piperidin-4-yl)), 3-pyridin-3-yl-beta-alanine (H-μAla(3-pyrid-3-yl)-OH), 2-(1-carbamimidoylpiperidin-4-yl)-glycine (Gly(1-carbamimidoylpiperidin-4-yl)), 3-amino-3-(1-carbamimidoylpiperidin-3-yl)propanoic acid, phenylglycine (Phg), 4-fluorophenylalanine (Phe(4-F)), 4-aminophenylalanine (Phe(4-NH₂)), 4-nitrophenylalanine (Phe(4-NO₂)), 4-carbamoylphenylalanine, 4-guanidinophenylalanine, asparagine (Asn), glutamic acid (Glu), proline, 4-ureidophenylalanine, (2-aminoethoxy)phenylalanine (Phe(4-OCH₂CH₂NH₂)), (pyridin-4-yl)alanine (3-Pal), (pyridin-4-yl)alanine (4-Pal), 3-(6-aminopyridin-3-yl)alanine ((3-Pal(6-NH₂)), 2-((2-aminopyridin-4-yl)-λ²-azaneyl)ethyl alanine, N6-nicotinoyl-Lys, 2-oxo-2-(piperazin-1-yl)ethyl alanine, 2-(piperidin-4-yl)ethylalanine, 3-((piperazin-1-yl)propyl)-glycine, 3-((4-methylpiperazin-1-yl)propyl)-glycine, 3-(4-benzylpiperidin-1-yl)propylglycine, 3-((4-benzylpiperazin-1-yl)propyl)-glycine, N6-(1-benzylpiperidine-4-carbonyl)-Lys, 3-((3,4-dihydroisoquinolin-2(1H)-yl)propyl)-glycine, 2-pyridin-4-ylthynyl alanine, (4-(aminomethyl)-1H-1,2λ²,3λ²-triazol-1-yl)methyl)-glycine, (4-(piperazin-1-ylmethyl)-1H-1,2λ²,3λ²-triazol-1-yl)methyl)-glycine, 2-((1H-imidazol-4-yl)ethyl)glycine, or (4-((R)-2,6-dioxohexahydropyrimidine-4-carboxamido)phenyl)alanine:
or X⁴ is 4-methylpiperazine-2-carboxylic acid,
or X⁴ is

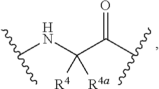

where R⁴ and R^{4a} are taken together with the carbon atom to which they are attached to form

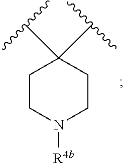

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

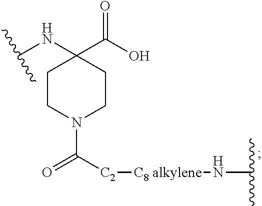

X⁵ is absent, -L⁵-, alanine (Ala), lysine (Lys), omithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-I)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, 4-amino-1-(methylglycyl)piperidine-4-carboxylic acid, N6-(4-fluorobenzoyl)-Lys, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

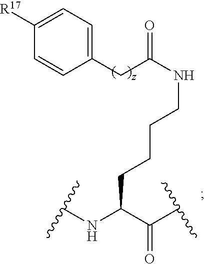

R¹⁷ is —CH₃ or —I;
z is 1, 2, or 3; and
X⁸ is absent, lysine (Lys), or glutamic acid (Glu);
L¹, L⁶, and L⁷ are each independently —NR¹⁵-L¹³-L¹⁴-C(═O)— or substituted or unsubstituted piperazinylene-;
   L¹³ is absent, —C₁-C₆ alkylene, substituted or unsubstituted heterocycloalkylene, substituted or unsubstituted cyclohexylene, —(CH₂CH₂O)_{y}—, or —(CH₂CH₂NR¹⁶)_{y}—;
   L¹⁴ is absent, —C₁-C₆ alkylene, substituted or unsubstituted heterocycloalkylene, or substituted or unsubstituted cyclohexylene;
   R¹⁵ and R¹⁶ are each independently selected from −1 or —C₁-C₄ alkylene;
   y is 1, 2, 3, 4, 5, or 6; and
wherein the compound of Formula (I) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃;
wherein the C-terminal end of the compound of Formula (VII) may be either —COOH or —CONH₂; and
wherein the compound of Formula (VII) is not:

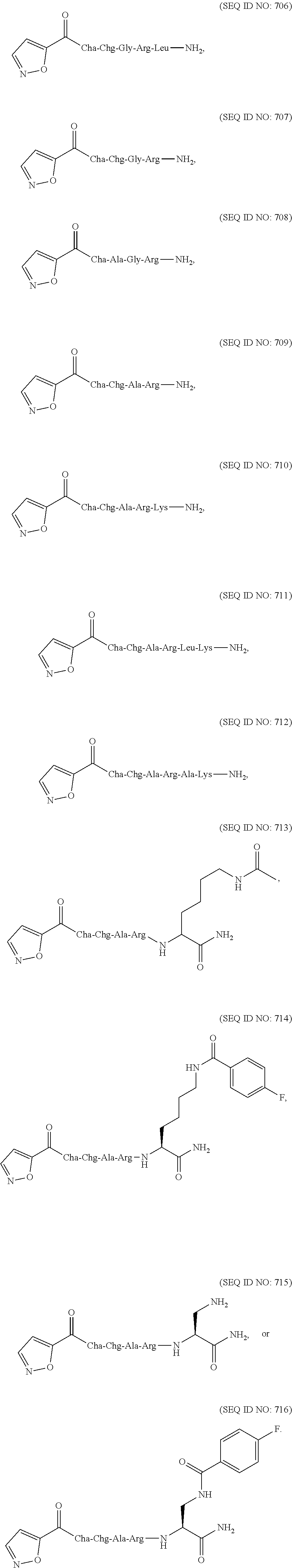

In some embodiments, described herein is a compound of Formula (VII), or a pharmaceutically acceptable salt thereof:

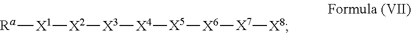

wherein:
R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
X¹ is

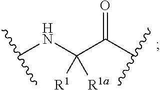

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each Rib is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

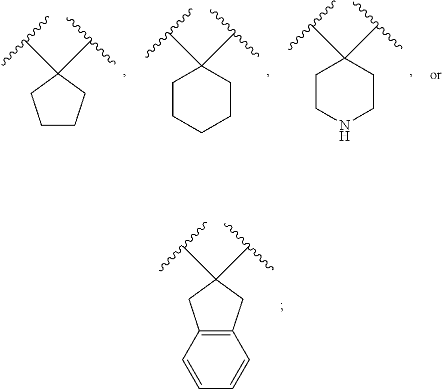 [SMILES: *C1(*)CCCC1 ; *C1(*)CCCCC1 ; *C1(*)CCNCC1 ; *C1(*)Cc2ccccc2C1]

X² is

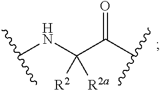

R² is -L²-R^{2b};
L² is absent, —CH₂—, or —CHPh-;
R^{2b} is pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

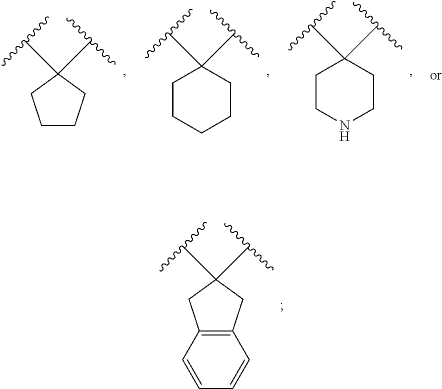 [SMILES: *C1(*)CCCC1 ; *C1(*)CCCCC1 ; *C1(*)CCNCC1 ; *C1(*)Cc2ccccc2C1]

X³ is alanine (Ala), trifluoromethylalanine, homoalanine, glutamic acid (Glu), glutamine (Gln), ornithine (Orn), lysine (Lys), serine (Ser), N-methylalanine (N-Me-Ala), 2-amino-2-methylpropanoic acid (AIB), 1-aminocyclopropane-1-carboxylic acid (ACC), norleucine (Nle), 3-aminoalanine, 2-amino-6-azidohexanoic acid, 2-amino-6-(4-hexyl-1H-1,2,3-triazol-1-yl)hexanoic acid, 2-amino-6-(4-((2-methoxyethoxy)methyl)-1H-1,2,3-triazol-1-yl)hexanoic acid, 2-amino-6-(4-(cyclopentylmethyl)-1H-1,2,3-triazol-1-yl)hexanoic acid, 2-amino-6-(4-benzyl-1H-1,2,3-triazol-1-yl)hexanoic acid, proline (Pro), cyclopropylglycine, or biphenylalanine (Bip);
X⁴ is arginine (Arg), 3-amino-6-guanidinohexanoic acid, N6-carbamimidoyl-lysine, 2-amino-5-ureidopentanoic acid, homoarginine, N-methyl-arginine, N^{ω}-methyl-arginine, N^{ω}-nitro-arginine, (E)-N^{ω},N^{ω}′-dimethyl-arginine, (E)-N^{ω},N^{ω}′-diethyl-arginine, ornithine (Orn), lysine, phenylalanine (Phe), cyclohexylglycine (Chg), cyclohexylalanine (Cha), norleucine (Nle), 3-amino-alanine (H-Dap-OH), piperidinyl-1-alanine, N⁴,N⁴-dimethyl-asparagine (N⁴,N⁴-dimethyl-Asn), 2-(piperidin-4-yl)-glycine (Gly(piperidin-4-yl)), 3-pyridin-3-yl-beta-alanine (H-βAla(3-pyrid-3-yl)-OH), 2-(1-carbamimidoylpiperidin-4-yl)-glycine (Gly(1-carbamimidoylpiperidin-4-yl)), 3-amino-3-(1-carbamimidoylpiperidin-3-yl)propanoic acid, phenylglycine (Phg), 4-fluorophenylalanine (Phe(4-F)), 4-aminophenylalanine (Phe(4-NH₂)), 4-nitrophenylalanine (Phe(4-NO₂)), 4-carbarnoylphenylalanine, 4-guanidinophenylalanine, asparagine (Asn), glutamic acid (Glu), proline, 4-ureidophenylalanine, (2-aminoethoxy)phenylalanine (Phe(4-OCH₂CH₂NH₂)), (pyridin-4-yl)alanine (3-Pal), (pyridin-4-yl)alanine (4-Pal), 3-(6-aminopyridin-3-yl)alanine ((3-Pal(6-NH₂)), 2-((2-aminopyridin-4-yl)-λ²-azaneyl)ethyl alanine, N6-nicotinoyl-Lys, 2-oxo-2-(piperazin-1-yl)ethyl alanine, 2-(piperidin-4-yl)ethylalanine, 3-((piperazin-1-yl)propyl)-glycine, 3-((4-methylpiperazin-1-yl)propyl)-glycine, 3-(4-benzylpiperidine-1-yl)propylglycine, 3-((4-benzylpiperazin-1-yl)propyl)-glycine, N6-(1-benzylpiperidine-4-carbonyl)-Lys, 3-((3,4-dihydroisoquinolin-2(1H)-yl)propyl)-glycine, 2-pyridin-4-ylthynyl alanine, (4-(aminomethyl)-1H-1,2λ²,3λ²-triazol-1-yl)methyl)-glycine, (4-(piperazin-1-ylmethyl)-1H-1,2λ²,3λ²-triazol-1-yl)methyl)-glycine, 2-((1H-imidazol-4-yl)ethyl)glycine, or (4-((R)-2,6-dioxohexahydropyrimidine-4-carboxamido)phenyl)alanine;
or X⁴ is 4-methylpiperazine-2-carboxylic acid;
or X⁴ is

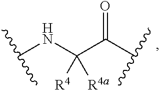

where R⁴ and R^{4a} are taken together with the carbon atom to which they are attached to form

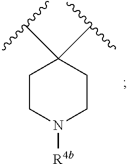

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

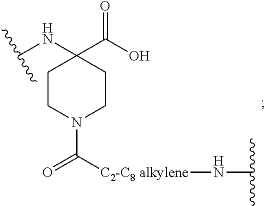

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-I)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

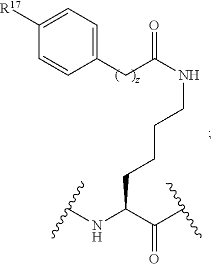

R¹⁷ is —CH₃ or —I;
z is 1, 2, or 3; and
X⁸ is absent, lysine (Lys), or glutamic acid (Glu);
L⁵, L⁶, and L⁷ are each independently —NR⁵-L¹³-L¹⁴-C(═O)— or substituted or unsubstituted piperazinylene-;
   L¹³ is absent, —C₁-C₆ alkylene, substituted or unsubstituted heterocycloalkylene, or substituted or unsubstituted cyclohexylene, —(CH₂CH₂O)_{y}—, or —(CH₂CH₂NR¹⁶)_{y}—;
   L¹⁴ is absent, —C₁-C₆ alkylene, substituted or unsubstituted heterocycloalkylene, or substituted or unsubstituted cyclohexylene; and
   R¹⁵ and R¹⁶ are each independently selected from H or —C₁-C₄ alkylene;
   y is 1, 2, 3, 4, 5, or 6;
wherein the compound of Formula (VII) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃;
wherein the C-terminal end of the compound of Formula (1) may be either —COOH or —CONH₂; and
wherein when X⁴ is Arginine, X⁵ is not absent, lysine (Lys), leucine (Leu), alanine (Ala), N6-acetyl-lysine, N6-(4-fluorobenzoyl)-Lys, 3-amino-alanine (H-Dap-O—I), or (4-fluorobenzamido)methylglycine.

In some embodiments, described herein is a compound of Formula (VII), or a pharmaceutically acceptable salt thereof:

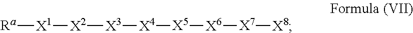

wherein:
R^{a} is —C(═O) R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹—;
wherein any saturated carbon of R¹⁰ is optionally substituted with oxo (=O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
X¹ is

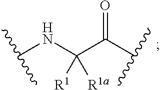

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

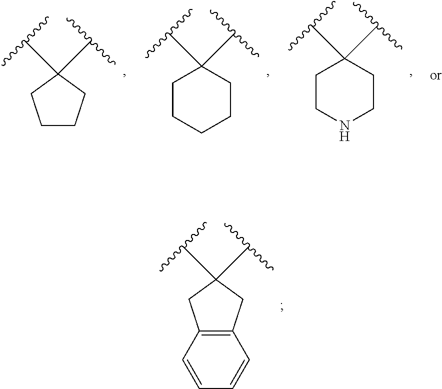 [SMILES: *C1(*)CCCC1 ; *C1(*)CCCCC1 ; *C1(*)CCNCC1 ; *C1(*)Cc2ccccc2C1]

X² is

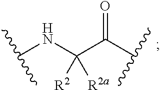

R² is -L²-R^{2b};
L² is absent, —CH₂—, or —CHPh-;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

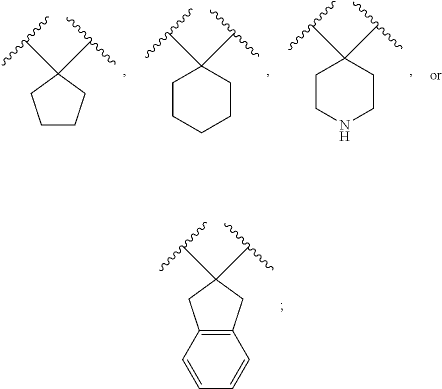 [SMILES: *C1(*)CCCC1 ; *C1(*)CCCCC1 ; *C1(*)CCNCC1 ; *C1(*)Cc2ccccc2C1]

X³ is glycine, alanine (Ala), trifluoromethylalanine, homoalanine, glutamic acid (Glu), glutamine (Gln), ornithine (Orn), lysine (Lys), serine (Ser), N-methylalanine (N-Me-Ala), 2-amino-2-methylpropanoic acid (AIB), 1-aminocyclopropane-1-carboxylic acid (ACC), norleucine (Nle), 3-aminoalanine, 2-amino-6-azidohexanoic acid, 2-amino-6-(4-hexyl-1H-1,2,3-triazol-1-yl)hexanoic acid, 2-amino-6-(4-((2-methoxyethoxy)methyl)-1H-1,2,3-triazol-1-yl)hexanoic acid, 2-amino-6-(4-(cyclopentylmethyl)-1H-1,2,3-triazol-1-yl)hexanoic acid, 2-amino-6-(4-benzyl-1H-1,2,3-triazol-l-yl)hexanoic acid, proline (Pro), cyclopropylglycine, or biphenylalanine (Bip);
X⁴ is 3-amino-6-guanidinohexanoic acid, N6-carbamimidoyl-lysine, 2-amino-5-ureidopentanoic acid, homoarginine, N-methyl-arginine, N^{ω}-methyl-arginine, N^{ω}-nitro-arginine, (E)-N^{ω},N^{ω}′-dimethyl-arginine, (E)-N^{ω},N^{ω}′-diethyl-arginine, ornithine (Orn), lysine, phenylalanine (Phe), cyclohexylglycine (Chg), cyclohexylalanine (Cha), norleucine (Nle), 3-amino-alanine (H-Dap-OH), piperidinyl-1-alanine, N⁴,N⁴-dimethyl-asparagine (N⁴,N⁴-dimethyl-Asn), 2-(piperidin-4-yl)-glycine (Gly(piperidin-4-yl)), 3-pyridin-3-yl-beta-alanine (H-βAla(3-pyrid-3-yl)-OH), 2-(1-carbamimidoylpiperidin-4-yl)-glycine (Gly(1-carbamimidoylpiperidin-4-yl)), 3-amino-3-(1-carbamimidoylpiperidin-3-yl)propanoic acid, phenylglycine (Phg), 4-fluorophenylalanine (Phe(4-F)), 4-aminophenylalanine (Phe(4-NH₂)), 4-nitrophenylalanine (Phe(4-NO₂)), 4-carbamoylphenylalanine, 4-guanidinophenylalanine, asparagine (Asn), glutamic acid (Glu), proline, 4-ureidophenylalanine, (2-aminoethoxy)phenylalanine (Phe(4-OCH₂CH₂NH₂)), (pyridin-4-yl)alanine (3-Pal), (pyridin-4-yl)alanine (4-Pal), 3-(6-aminopyridin-3-yl)alanine ((3-Pal(6-NH₂)), 2-((2-aminopyridin-4-yl)-λ²-azaneyl)ethyl alanine, N6-nicotinoyl-Lys, 2-oxo-2-(piperazin-1-yl)ethyl alanine, 2-(piperidin-4-yl)ethylalanine, 3-((piperazin-1-yl)propyl)-glycine, 3-((4-methylpiperazin-1-yl)propyl)-glycine, 3-(4-benzylpiperidin-1-yl)propylglycine, 3-((4-benzylpiperazin-1-yl)propyl)-glycine, N6-(1-benzylpiperidine-4-carbonyl)-Lys, 3-((3,4-dihydroisoquinolin-2(1H)-yl)propyl)-glycine, 2-pyridin-4-ylthynyl alanine, (4-(aminomethyl)-1H-1,2λ²,3λ²-triazol-1-yl)methyl)-glycine, (4-(piperazin-1-ylmethyl)-1H-1,2λ²,3λ²-triazol-1-yl)methyl)-glycine, 2-((1H-imidazol-4-yl)ethyl)glycine, or (4-((R)-2,6-dioxohexahydropyrimidine-4-carboxamido)phenyl)alanine;
or X⁴ is 4-methylpiperazine-2-carboxylic acid;
or X⁴ is

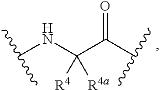

where R⁴ and R^{4a} are taken together with the carbon atom to which they are attached to form

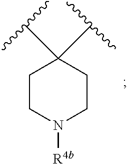

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

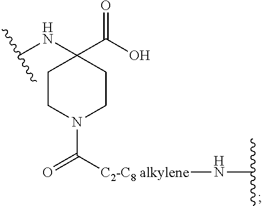

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N3)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-I)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4—CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L^{h}-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

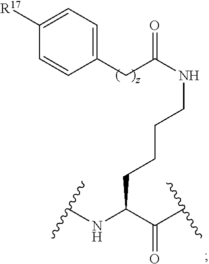

R¹⁷ is —CH₃ or —I;
z is 1, 2, or 3; and
X⁸ is absent, lysine (Lys), or glutamic acid (Glu);
L⁵, L⁶, and L⁷ are each independently —NR¹⁵-L¹³-L¹⁴-C(═O)— or substituted or unsubstituted piperazinylene-;
   L¹³ is absent, —C₁-C₆ alkylene, substituted or unsubstituted heterocycloalkylene, or substituted or unsubstituted cyclohexylene, —(CH₂CH₂O)_{y}—, or —(CH₂CH₂NR¹⁶)_{y}—;
   L¹⁴ is absent, —C₁-C₆ alkylene, substituted or unsubstituted heterocycloalkylene, or substituted or unsubstituted cyclohexylene;
   R¹⁵ and R¹⁶ are each independently selected from H or —C₁-C₄ alkylene; and
y is 1, 2, 3, 4, 5, or 6;
wherein the compound of Formula (I) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (I) may be either —COOH or —CONH₂.

Also described herein is a pharmaceutical composition comprising a compound described herein (e.g., a compound of Formula (VII), or a pharmaceutically acceptable salt thereof, and at least one pharmaceutically acceptable excipient. In some embodiments, the pharmaceutical composition is formulated for administration to a mammal by oral administration.

In another aspect, described herein is a method for the treatment of a chronic inflammatory condition comprising administering to a mammal with a chronic inflammatory condition an effective amount of a compound described herein (e.g. a compound of Formula (VII)), or a pharmaceutically acceptable salt thereof, or an effective amount of pharmaceutical composition comprising a compound described herein (e.g., a compound of Formula (VII)), or a pharmaceutically acceptable salt thereof.

In another aspect, described herein is a method for the treatment of direct tissue damage comprising administering to a mammal with a chronic inflammatory condition an effective amount of a compound described herein (e.g. a compound of Formula (VII)), or a pharmaceutically acceptable salt thereof, or an effective amount of pharmaceutical composition comprising a compound described herein (e.g., a compound of Formula (VII)), or a pharmaceutically acceptable salt thereof.

In another aspect, described herein is a use of a compound described herein (e.g. a compound of Formula (VII)) in the preparation of a bifunctional molecule, wherein the bifunctional molecule comprises:
A) a radionuclide conjugate comprising a compound of Formula (VII), an optional linker attached to the Compound of Formula (VII), a chelating moiety attached to the linker, and optionally a radionuclide complexed by the chelating moiety;
B) a peptide-drug conjugate comprising a compound of Formula (VII), an optional linker attached to the Compound of Formula (VII), and a toxin attached to the optional linker; or
a peptide-antibody conjugate consisting of a compound of Formula (VII), an optional linker attached to the Compound of Formula (VII), and an antibody attached to the optional linker.

In any of the embodiments disclosed herein, the mammal is a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1 depicts biodistribution of ¹¹¹In[In]-Compound 240 in tumor bearing Swiss nude mice. Timepoints are 0.5 h, 2.0 h, 5.0 h, 24 h and 72 h post IV treatment. Activity is measured as percentage of injected dose per gram of tissue (% ID/g).

FIG. 2 depicts competition study of ¹¹¹In[Ln]-Compound 240 co-administered with excessive unlabeled Compound 240 (1:100 ratio respectively) in tumor bearing Swiss nude mice. Timepoint is 2h. Activity is measured as percentage of injected dose per gram of tissue (% ID/g).

FIG. 3 depicts biodistribution of ¹¹¹In[In]-Compound 250 in tumor bearing Swiss nude mice. Timepoints are 0.5 h, 2.0 b, 5.0 h, 241 h and 72 h post IV treatment. Activity is measured as percentage of injected dose per gram of tissue (% ID/g).

FIG. 4 depicts competition study of ¹¹¹In[In]-Compound 250 co-administered with excessive unlabeled Compound 250 (1:100 ratio respectively) in tumor bearing Swiss nude mice. Timepoint is 2 h. Activity is measured as percentage of injected dose per gram of tissue (% ID/g).

FIG. 5 depicts biodistribution of ¹¹¹In[In]-Compound 402 in tumor bearing Swiss nude mice. Timepoints are 0.5 h, 2.0 h, 5.0 h, 24 h and 72 h post IV treatment. Activity is measured as percentage of injected dose per gram of tissue (% ID/g).

FIG. 6 depicts competition study of ¹¹¹In[In]-Compound 402 co-administered with excessive ¹¹⁵In[In]-Compound 402 (1:100 ratio respectively) in tumor bearing Swiss nude mice. The timepoints is 2-3h. Activity is measured as percentage of injected dose per gram of tissue (% ID/g).

FIG. 7 depicts biodistribution of ¹⁷⁷Lu[Lu]-Compound 526 in tumor bearing Swiss nude mice. Timepoints are 0.5, 2.0, 5.0, 24, 72 and 168 h post IV treatment. Activity is measured as percentage of injected dose per gram of tissue (% ID/g).

FIG. 8 depicts competition study of ¹⁷⁷Lu[Lu]-Compound 526 co-administered with excessive ¹⁷⁵Lu[Lu]-Compound 526 (1:100 ratio respectively) in tumor bearing Swiss nude mice. The timepoint is 2h. Activity is measured as percentage of injected dose per gram of tissue (% ID/g).

### DETAILED DESCRIPTION OF THE INVENTION

Cancer, a disease in which some cells undergo a genetic change in the control of their growth and replication that results in uncontrolled growth and spreading, is one of the leading causes of death worldwide. General types of cancers include solid tumors (cancers that typically originate in organs), carcinomas (cancers that originate in skin or tissues that line organs), sarcomas (cancers of connective tissues such as bones), leukemias (cancers of bone marrow), and lymphomas and myelomas (cancers of the immune system). Neoplasms are abnormal growth of cells that result in solid tumors which may be benign (i.e., do not display malignant features and are generally unlikely to become dangerous such as adenomas), malignant (i.e., display features such as genetic mutations, loss of normal function, rapid division, and ability metastasize (invade) to other tissues), and of uncertain or unknown behavior. State-of-the-art treatment of neoplasms is accomplished by a combination of surgical procedures, chemotherapy, and radiation therapy. Surgical procedures can be curative under some conditions, but often require multiple interventions and are often done in combination with radiation and chemotherapy. Chemotherapy proves to be a potent weapon in the fight against cancer in many cases. Chemotherapy is typically performed by systemic administration of potent cytotoxic drugs, but these compounds often lack tumor selectivity and therefore also kill healthy cells in the body. The resulting non-specific toxicity is the cause of severe side effects of chemotherapy which occur because chemotherapy does not target the cancerous cells specifically over other cells. Radiotherapy is the use of high-energy radiation to kill cells. The source of radiation may be external-beam radiation (applied using an external source), internal radiation (placement of a radioactive material near the target cells), or radiotherapy from the systemic administration of a radioactive material. Like chemotherapy, many radiation therapy options also lack tumor cell identification properties needed to achieve the ultimate goal of targeted tumor therapy with drug molecules or radionuclides.

Described herein are radiopharmaceuticals that selectively deliver radionuclides to malignant cells that overexpress PAR2 for use in cancer detection, image guided cancer surgery, and selective tumor killing.

The Protease activated receptor 2 (PAR2)

Protease-activated receptors (PARs) are a class of G protein-coupled seven transmembrane receptors (GPCRs) that are self-activated by a tethered ligand. A tethered ligand is a component of a receptor that is revealed, binds to, and activates the receptor after a specific protease cleaves an N-terminal portion. PAR2 is one of four PAR subtypes that is activated by various trypsin-like serine proteases (e.g., trypsin, tryptase, granzyme A, KLK4), whereas PAR1, PAR3, and PAR4 are activated by thrombin.

PAR2, also known as coagulation factor II (thrombin) receptor-like 1 (F2RL1) or G-Protein coupled receptor I1 (GPR11), is encoded by the F2RL1 gene. The ligand sequences SLIGKV (in humans) (SEQ ID NO: 725) and SLIGRL (in rodents) (SEQ ID NO: 726) have been determined as the relevant amino acid sequences that induce binding and activation of PAR2.

Multiple tissues (e.g., pancreas, liver, small intestine, colon) endogenously express PAR2 as it is involved in inflammation and cell migration. However, inappropriate expression and abnormal function of PAR2 has been linked to various cancers and inflammatory diseases, such as arthritis, colitis, asthma, cardiovascular disease, prostate cancer, lung cancer, gastric cancer, liver cancer, colorectal cancer, melanoma, ovarian cancer, and breast cancer. There is up to a 16-fold increase in PAR2 expression in various cancers and PAR2 expression levels have been correlated to cancer staging, progression, and prognosis.

Radiopharmaceuticals targeting PAR2 are important for the development of new cancer therapies.

Solid Tumors: Benign and/or Malignant Neoplasms (Cancer)

In one aspect, the PAR2-targeted radiopharmaceuticals described herein are used to treat benign and/or malignant neoplasms (solid tumors), wherein the neoplasm comprises cells that overexpress PAR2 on the cell surface.

The term “neoplasm” as used herein, refers to an abnormal growth of cells that may proliferate in an uncontrolled way and may have the ability to metastasize (spread).

Neoplasms include solid tumors, adenomas, carcinomas, sarcomas, leukemias and lymphomas, at any stage of the disease with or without metastases.

A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

Solid tumors are cancers that typically originate in organs, such as the bladder, bowel, brain, breast, endometrium, heart, kidney, lung, liver, uterus, ovaries, pancreas or other endocrine organs (thyroid), and prostate.

In some embodiments, the PAR2-targeted radiopharmaceuticals described herein are used to treat an adenoma. An adenoma is a tumor that is not cancer. It starts in gland-like cells of the epithelial tissue (thin layer of tissue that covers organs, glands, and other structures within the body). An adenoma can grow from many glandular organs, including the adrenal glands, pituitary gland, thyroid, prostate, and others Even though benign, they have the potential to cause serious health complications by compressing other structures (mass effect) and by producing large amounts of hormones in an unregulated, non-feedback-dependent manner (causing paraneoplastic syndromes). Overtime adenomas may transform to become malignant, at which point they are called adenocarcinomas.

Adenomas may be found in the colon (e.g. adenomatous polyps, which have a tendency to become malignant and to lead to colon cancer), kidneys (e.g. renal adenomas may be precursor lesions to renal carcinomas), adrenal glands (e.g. adrenal adenomas; some secrete hormones such as cortisol, causing Cushing's syndrome, aldosterone causing Conn's syndrome, or androgens causing hyperandrogenism), thyroid (e.g. thyroid adenoma), pituitary (e.g. pituitary adenomas, such as prolactinoma, Cushing's disease and acromegaly), parathyroid (e.g. an adenoma of a parathyroid gland may secrete inappropriately high amounts of parathyroid hormone and thereby cause primary hyperparathyroidism), liver (e.g. hepatocellular adenoma), breast (e.g. fibroadenomas), appendix (e.g. cystadenoma), bronchial (e.g. bronchial adenomas may cause carcinoid syndrome, a type of paraneoplastic syndrome), prostate (e.g. prostate adenoma), sebaceous gland (e.g. sebaceous adenoma), and salivary glands.

Metastasis is the spread of malignant cells to new areas of the body, often by way of the lymph system or bloodstream. A metastatic tumor is one that has spread from the primary site of origin, or where it started, into different areas of the body. Metastatic tumors comprise malignant cells that may express cell surface PAR2.

Tumors formed from cells that have spread are called secondary tumors. Tumors may have spread to areas near the primary site, called regional metastasis, or to parts of the body that are farther away, called distant metastasis.

In some embodiments, the tumor to be treated comprises tumor cells expressing PAR2, wherein the tumor is a primary or metastatic tumor. In some embodiments, the tumor to be treated comprises tumor cells expressing PAR2, wherein the tumor is a primary or metastatic tumor of colon origin. In some embodiments, the tumor to be treated comprises tumor cells expressing PAR2, wherein the tumor is a primary or metastatic tumor of pancreatic origin. In some embodiments, the tumor to be treated comprises tumor cells expressing PAR2, wherein the tumor is a primary or metastatic tumor of stomach origin. In some embodiments, the tumor to be treated comprises tumor cells expressing PAR2, wherein the tumor is a primary or metastatic tumor of esophageal origin. In some embodiments, the tumor to be treated comprises tumor cells expressing PAR2, wherein the tumor is a primary or metastatic tumor of rectal origin. In some embodiments, the tumor to be treated comprises tumor cells expressing PAR2, wherein the tumor is a primary or metastatic tumor of liver origin. In some embodiments, the tumor to be treated comprises tumor cells expressing PAR2, wherein the tumor is a primary or metastatic tumor of lung origin.

In some embodiments, the PAR2-targeted radiopharmaceuticals described herein are used to treat a carcinoma. Carcinomas include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc. In some embodiments, the PAR2-targeted radiopharmaceuticals described herein are used to treat breast carcinoma. In some embodiments, the PAR2-targeted radiopharmaceuticals described herein are used to treat renal cell carcinoma. In some embodiments, the PAR2-targeted radiopharmaceuticals described herein are used to treat lung carcinoma.

In some embodiments, the PAR2-targeted radiopharmaceuticals described herein are used to treat a sarcoma. Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Solid tumors include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Benign solid tumors include adenomas.

Primary and metastatic tumors include, e.g., lung cancer (including, but not limited to, lung adenocarcinoma, squamous cell carcinoma, large cell carcinoma, bronchioloalveolar carcinoma, non-small-cell carcinoma, small cell carcinoma, mesothelioma); breast cancer (including, but not limited to, ductal carcinoma, lobular carcinoma, inflammatory breast cancer, clear cell carcinoma, mucinous carcinoma); colorectal cancer (including, but not limited to, colon cancer, rectal cancer); anal cancer; pancreatic cancer (including, but not limited to, pancreatic adenocarcinoma, islet cell carcinoma, neuroendocrine tumors); prostate cancer; ovarian carcinoma (including, but not limited to, ovarian epithelial carcinoma or surface epithelial-stromal tumor including serous tumor, endometrioid tumor and mucinous cystadenocarcinoma, sex-cord-stromal tumor); liver and bile duct carcinoma (including, but not limited to, hepatocellular carcinoma, cholangiocarcinoma, hemangioma); esophageal carcinoma (including, but not limited to, esophageal adenocarcinoma and squamous cell carcinoma); non-Hodgkin's lymphoma; bladder carcinoma; carcinoma of the uterus (including, but not limited to, endometrial adenocarcinoma, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including, but not limited to, renal cell carcinoma, clear cell carcinoma, Wilm's tumor); cancer of the head and neck (including, but not limited to, squamous cell carcinomas); cancer of the stomach (including, but not limited to, stomach adenocarcinoma, gastrointestinal stromal tumor); multiple myeloma; testicular cancer; germ cell tumor; neuroendocrine tumor; cervical cancer; carcinoids of the gastrointestinal tract, breast, and other organs; and signet ring cell carcinoma.

### Representative Protease-Activated Receptor-2 (PAR2) Targeting Conjugates

In some embodiments, the PAR2-targeted radiopharmaceuticals described herein have an affinity to PAR2 that is at least 10-fold, at least 50-fold, at least 100-fold, at least 200-fold, at least 500-fold, or at least 1000-fold greater than the affinity for other non-target receptors.

In some embodiments, the PAR2-targeted radiopharmaceuticals described herein preferentially accumulate in tumor tissues that express the targeted PAR2. In some embodiments, the PAR2-targeted radiopharmaceuticals described herein preferentially accumulate in tissues or organs comprising tumor cells that express PAR2 as compared to tissues or organ(s) lacking tumor cells that express PAR2. In some embodiments, the PAR2-targeted radiopharmaceuticals described herein preferentially accumulate at least 1-fold, at least 2-fold, at least 3-fold, at least 4-fold, at least 5-fold, or greater than 5-fold more in tissues or organ(s) comprising tumor cells that express PAR2 as compared to tissues or organs lacking tumor cells that express PAR2. It is understood that the compound may accumulate in certain tissues and organs involved in the metabolism and/or excretion of therapeutics, including but not limited to the kidneys and liver.

In one aspect, described herein is a compound of Formula (I), or a pharmaceutically acceptable salt thereof:

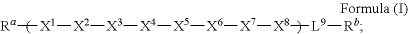

wherein:
R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
L⁹ is an optional linker that is attached to any one of X¹, X², X³, X⁴, X⁵, X⁶, X⁷ or X⁸;
R^{b} is a chelating moiety or a radionuclide complex thereof;
X¹ is

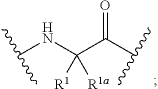

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

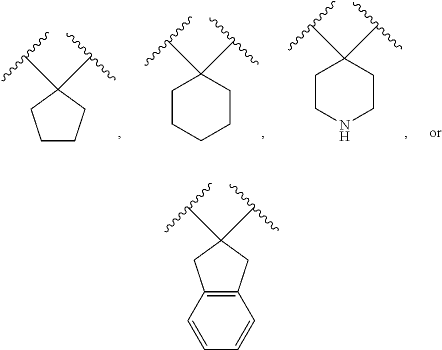 [SMILES: *C1(*)CCCC1 ; *C1(*)CCCCC1 ; *C1(*)CCNCC1 ; *C1(*)Cc2ccccc2C1]

X² is

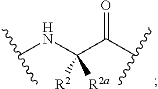

R² is -L²-R^{2b};
L² is absent, —CH₂—, or —CHPh-;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl,
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

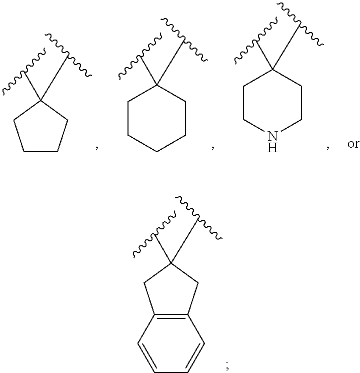 [SMILES: *C1(*)CCCC1 ; *C1(*)CCCCC1 ; *C1(*)CCNCC1 ; *C1(*)Cc2ccccc2C1]

X³ is glycine, (S)-alanine (Ala), (S)-trifluoromethylalanine, (S)-homoalanine, (S)-Glu, (S)-Gln, (S)-Orn, (S)-lysine (Lys), (S)-serine (Ser), (S)—N-methylalanine (N-Me-Ala), 2-amino-2-methylpropanoic acid (AIB), 1-aminocyclopropane-1-carboxylic acid (ACC), norleucine (Nle), (S)-3-aminoalanine, (S)-2-amino-6-azidohexanoic acid, (S)-2-amino-6-(4-hexyl-1H-1,2,3-triazol-1-yl)hexanoic acid, (S)-2-amino-6-(4-((2-methoxyethoxy)methyl)-1H-1,2,3-triazol-1-yl)hexanoic acid, (S)-2-amino-6-(4-(cyclopentylmethyl)-1H-1,2,3-triazol-1-yl)hexanoic acid, (S)-2-amino-6-(4-benzyl-1H-1,2,3-triazol-1-yl)hexanoic acid, (S)-proline (Pro), (S)-cyclopropylglycine, or (S)-biphenylalanine (Bip);
X⁴ is (S)-arginine (Arg), (S)-3-amino-6-guanidinohexanoic acid, N6-carbaamimidoyl-(S)-lysine, (S)-2-amino-5-ureidopentanoic acid, (S)-homoarginine, N-methyl-(S)-arginine, N^{ω}-methyl-(S)-arginine, N^{ω}-nitro-(S)-arginine, (E)-N^{ω},N^{ω}′-dimethyl-(S)-arginine, (E)-N^{ω},N^{ω}′-diethyl-(S)-arginine, (S)-ornithine (Orn), (S)-lysine, (S)-phenylalanine (Phe), (S)-cyclohexylglycine (Chg), (S)-cyclohexylalanine (Cha), norleucine (Nle), (S)-3-amino-alanine (H-Dap-OH), piperidinyl-1-yl alanine, N⁴,N⁴-dimethyl-(S)-asparagine (N⁴,N⁴-dimethyl-Asn), (S)-2-(piperidin-4-yl)-glycine (Gly(piperidin-4-yl)), (S)-3-pyridin-3-yl-beta-alanine (H-βAla(3-pyrid-3-yl)-OH), (S)-2-(1-carbamimidoylpiperidin-4-yl)-glycine (Gly(1-carbamimidoylpiperidin-4-yl)), (S)-3-amino-3-(1-carbamimidoylpiperidin-3-yl)propanoic acid, (S)-phenylglycine (Phg), (S)-4-fluorophenylalanine (Phe(4-F)), (S)-4-aminophenylalanine (Phe(4-NH₂)), (S)-4-nitrophenylalanine (Phe(4-NO₂)), (S)-4-carbamoylphenylalanine, (S)-4-guanidinophenylalanine, (S)-asparagine (Asn), (S)-glutamic acid (Glu), (S)-proline, (S)-4-ureidophenylalanine, (S)-(2-aminoethoxy)phenylalanine (Phe(4-OCH₂CH₂NH₂)), (S)-(pyridin-4-yl)alanine (3-Pal), (S)-(pyridin-4-yl)alanine (4-Pal), (S)-3-(6-aminopyridin-3-yl)alanine ((3-Pal(6-NH₂)), (S)-2-((2-aminopyridin-4-yl)-λ²-azaneyl)ethyl alanine, N6-nicotinoyl-Lys, (S)-2-oxo-2-(piperazin-1-yl)ethyl alanine, (S)-2-(piperidin-4-yl)ethyl alanine, (S)-3-((piperazin-1-yl)propyl)-glycine, (S)-3-((4-methylpiperazin-1-yl)propyl)-glycine, (S)-3-(4-benzylpiperidin-1-yl)propylglycine, (S)-3-((4-benzylpiperazin-1-yl)propyl)-glycine, N6-(1-benzylpiperidine-4-carbonyl)-Lys, (S)-3-((3,4-dihydroisoquinolin-2(1H)-yl)propyl)-glycine, (S)-2-pyridin-4-ylthynyl alanine, (S)-(4-(aminomethyl)-1H-1,2λ²,3λ²-triazol-1-yl)methyl)-glycine, (S)-(4-(piperazin-1-ylmethyl)-1H-1,2λ²,3λ²-triazol-1-yl)methyl)-glycine, (S)-2-((1H-imidazol-4-yl)ethyl)glycine, or (4-((R)-2,6-dioxohexahydropyrimidine-4-carboxamido)phenyl)alanine;
or X⁴ is (S)-4-methylpiperazine-2-carboxylic acid,
or X⁴ is

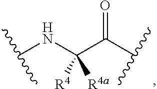

where R⁴ and R^{4a} are taken together with the carbon atom to which they are attached to form

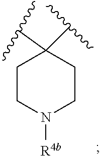

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

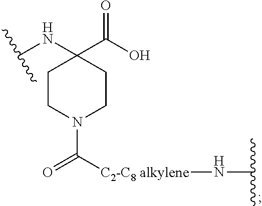

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid

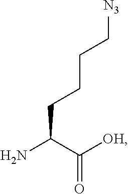 [SMILES: [N-]=[N+]=NCCCC[C@H](N)C(=O)O]

N6-acetyl-lysine

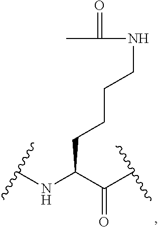 [SMILES: *N[C@@H](CCCCNC(C)=O)C(*)=O]

N6-(pent-4-ynoyl)-lysine

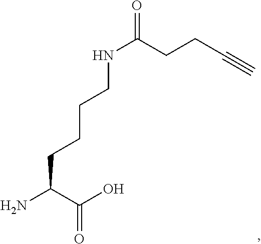 [SMILES: C#CCCC(=O)NCCCC[C@H](N)C(=O)O]

N6-octanoyl-lysine

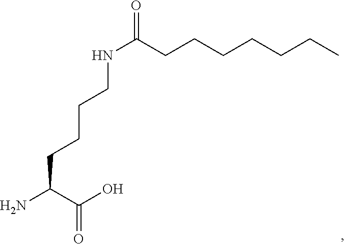 [SMILES: CCCCCCCC(=O)NCCCC[C@H](N)C(=O)O]

tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-I)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

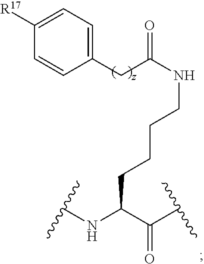

R¹⁷ is —CH₃ or —I;
z is 1, 2, or 3;
X⁸ is absent, lysine (Lys), or glutamic acid (Glu);
L⁵, L⁶, and L⁷ are each independently —NR¹⁵-L¹³-L¹⁴-C(═O)— or substituted or unsubstituted piperazinylene-;
L¹³ is absent, —C₁-C₆ alkylene, substituted or unsubstituted heterocycloalkylene, or substituted or unsubstituted cyclohexylene, —(CH₂CH₂O)_{y}—, or —(CH₂CH₂NR¹⁶)_{y}—;
L¹⁴ is absent, —C₁-C₆ alkylene, substituted or unsubstituted heterocycloalkylene, or substituted or unsubstituted cyclohexylene;
R¹⁵ and R¹⁶ are each independently selected from H or —C₁-C₄ alkyl; and
y is 1, 2, 3, 4, 5, or 6; and
wherein the compound of Formula (I) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
   wherein the C-terminal end of the compound of Formula (I) may be either —COOH or —CONH₂.

In some embodiments,
R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
L⁹ is an optional linker that is attached to any one of X⁵, X⁶, or X⁷;
R^{b} is a chelating moiety or a radionuclide complex thereof;
X¹ is

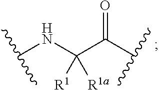

R¹ is -L¹-R^{1b}; L¹ is absent or —CH₂—; R^{1b} is cyclopentyl, cyclohexyl, cycloheptyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, 2-chlorophenyl, 3-chlorophenyl, 4-chlorophenyl, 2,4-dichlorophenyl, 3,4-dichlorophenyl, or 2,5-dichlorophenyl;
R^{1a} is hydrogen or methyl;
X² is

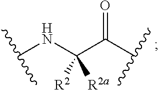

R² is -L²-R^{2b}; L² is absent or —CH₂—; R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl, or 4′-methyl-[1,1′-biphenyl]-4-yl;
R^{2a} is hydrogen or methyl;
X³ is glycine, (S)-alanine (Ala), or norleucine (Nle);
X⁴ is (S)-arginine (Arg), (S)-ornithine (Orn), (S)-phenylalanine (Phe), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-(pyridin-4-yl)alanine (4-Pal), (S)-3-(6-aminopyridin-3-yl)alanine ((3-Pal(6-NH₂)), or (S)-2-((2-aminopyridin-4-yl)-λ²-azaneyl)ethyl alanine;
X⁵ is alanine (Ala), lysine (Lys), ornithine (Orn),

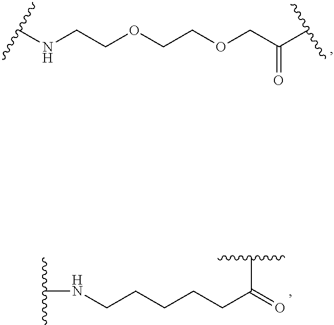 [SMILES: *NCCOCCOCC(*)=O ; *NCCCCCC(*)=O]

N6-(4-fluorobenzoyl)-Lys, β³-homolysine, 3-amino-alanine (H-Dap-OH), 4-nitrophenylalanine (Phe(4-NO₂)), or (4-fluorobenzamido)methylglycine;
X⁶ is absent, lysine (Lys), ornithine (Orn),

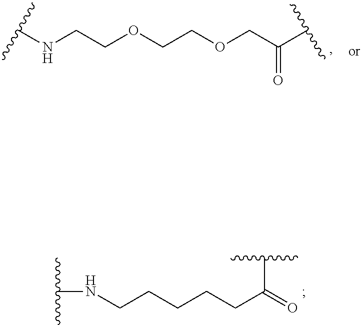 [SMILES: *NCCOCCOCC(*)=O ; *NCCCCCC(*)=O]

X⁷ is absent, lysine (Lys), ornithine (Orn), or

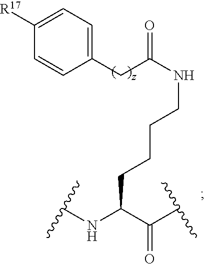

R¹⁷ is —CH₃ or —I;
z is 1, 2, or 3; and
X⁸ is absent;
wherein the compound of Formula (I) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (I) may be either —COOH or —CONH₂.

In some embodiments, the compound has the structure of Formula (Ia):

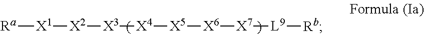

wherein L⁹ is an optional linker that is attached to any one of X⁴, X⁵, X⁶, or X⁷.

In some embodiments, the compound has the structure of Formula (Ib):

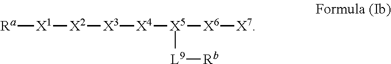

In some embodiments, L⁹ is a bond.

In some embodiments, X⁶ and X⁷ are absent.

In some embodiments, the compound of Formula (Tb) has the following structure:

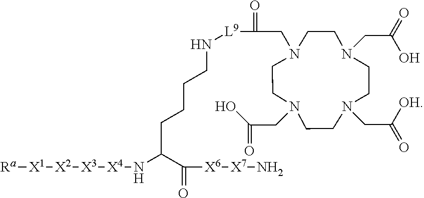

In some embodiments, the compound of Formula (Ib) has the following structure:

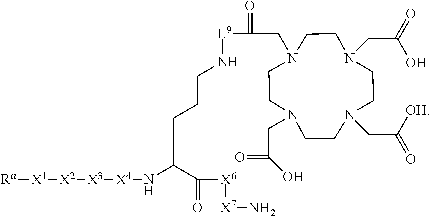

In some embodiments, the compound of Formula (Ib) has the following structure:

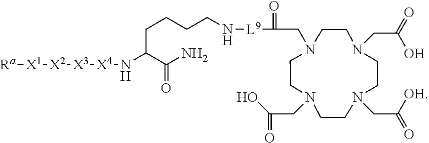

In some embodiments, the compound of Formula (Ib) has the following structure:

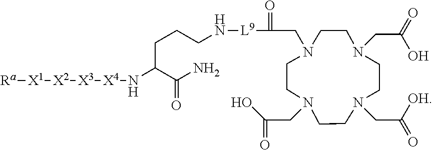

In some embodiments, the compound of Formula (Ib) has the following structure:

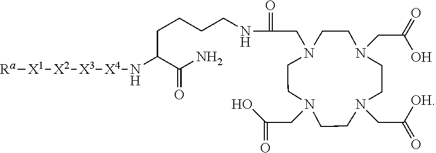

In some embodiments, the compound of Formula (Ib) has the following structure:

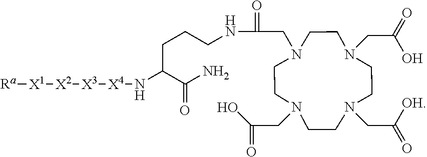

In some embodiments, the compound of Formula (Ib) has the following structure:

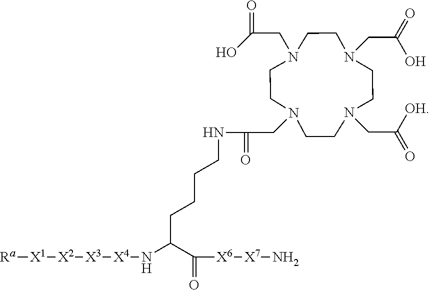

In some embodiments, the compound of Formula (Ib) has the following structure:

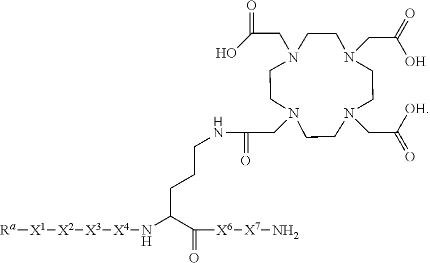

In some embodiments, the compound of Formula (Ib) has the following structure:

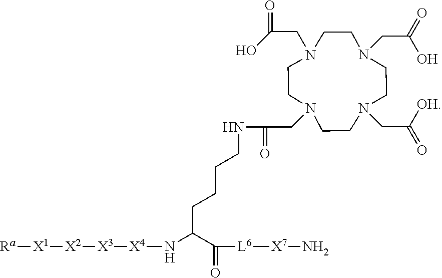

In some embodiments, the compound of Formula (Ib) has the following structure:

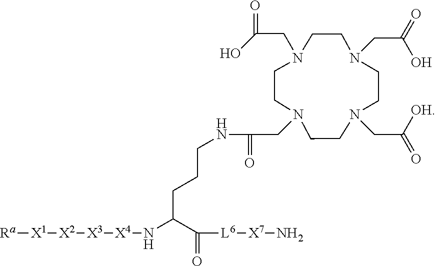

In some embodiments, the compound of Formula (Ib) has one of the following structures:

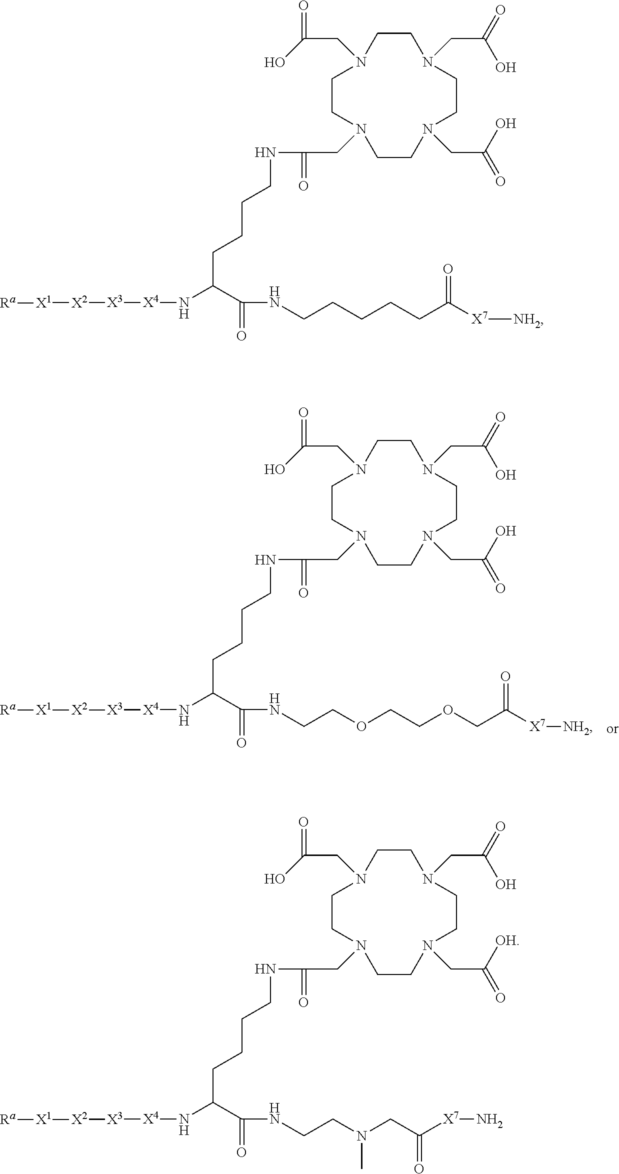

In some embodiments, the compound of Formula (Ib) has one of the following structures:

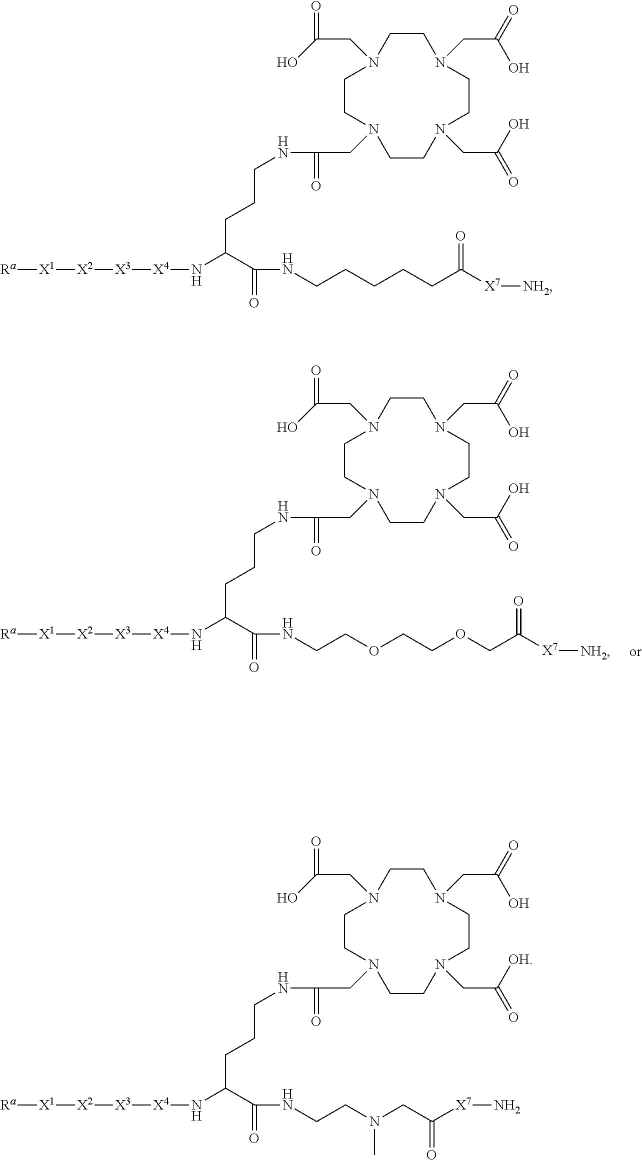

In some embodiments, the compound has the structure of Formula (Ic)

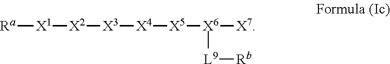

In some embodiments, the compound of Formula (Ic) has the following structure:

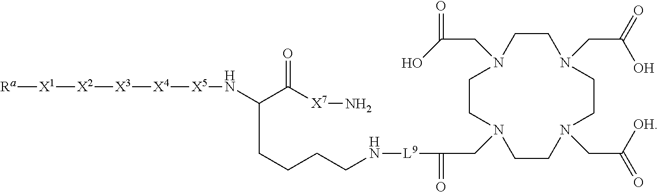

In some embodiments, the compound of Formula (Ic) has the following structure:

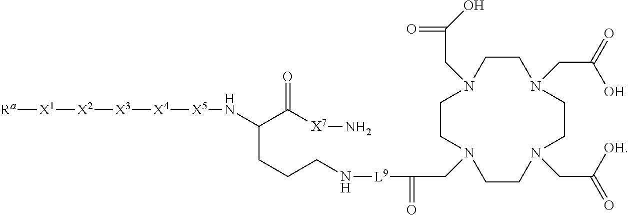

In some embodiments, the compound of Formula (Ic) has the following structure:

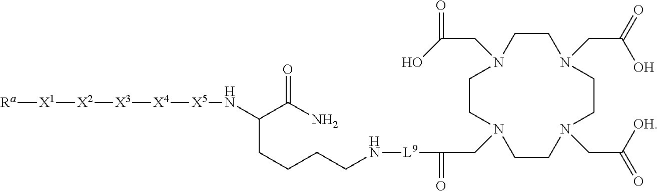

In some embodiments, the compound of Formula (Ic) has the following structure:

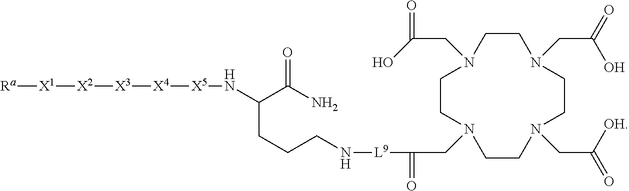

In some embodiments, the compound of Formula (Ic) has the following structure:

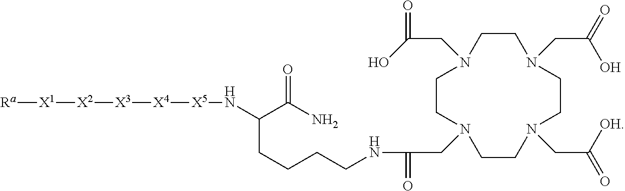

In some embodiments, the compound of Formula (Ic) has the following structure:

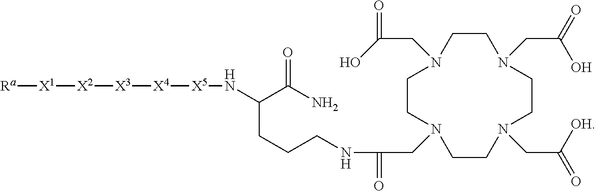

In some embodiments, the compound of Formula (Ic) has the following structure:

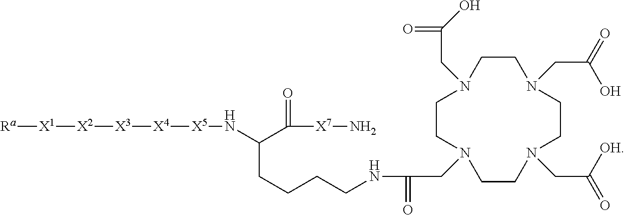

In some embodiments, the compound of Formula (Ic) has the following structure:

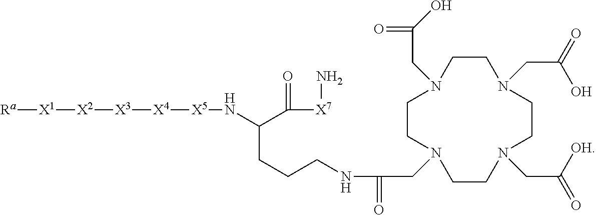

In some embodiments, the compound has the structure of Formula (Id):

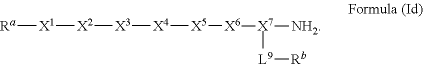

In some embodiments, the compound of Formula (Id) has the following structure:

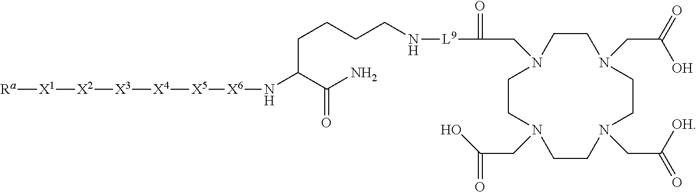

In some embodiments, the compound of Formula (Id) has the following structure:

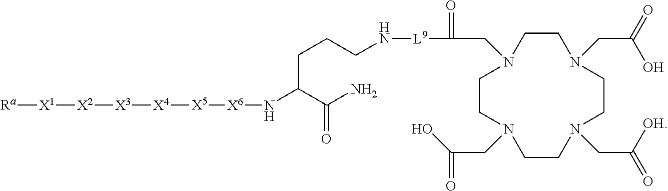

In some embodiments, the compound of Formula (Id) has the following structure:

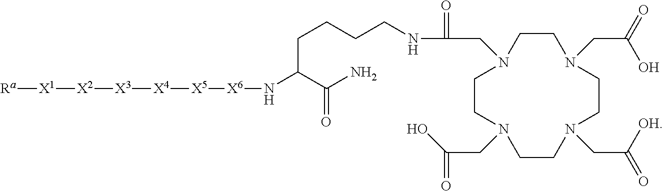

In some embodiments, the compound of Formula (Id) has the following structure:

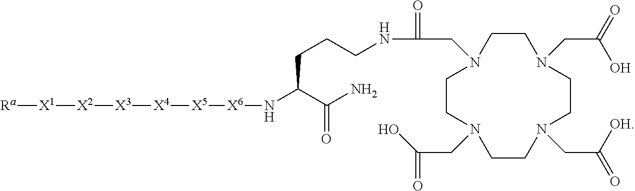

In some embodiments, the compound of Formula (Ib) has the following structure:

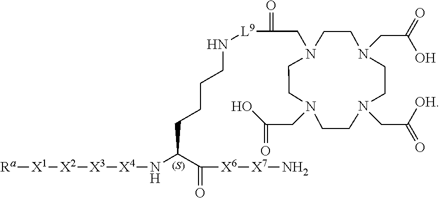

In some embodiments, the compound of Formula (Ib) has the following structure:

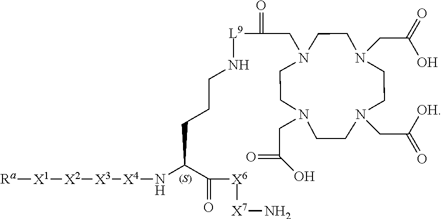

In some embodiments, the compound of Formula (Ib) has the following structure:

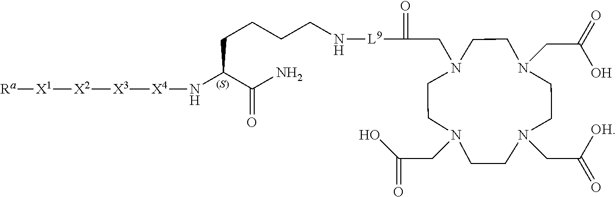

In some embodiments, the compound of Formula (Ib) has the following structure:

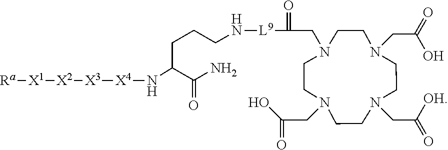

In some embodiments, the compound of Formula (Ib) has the following structure:

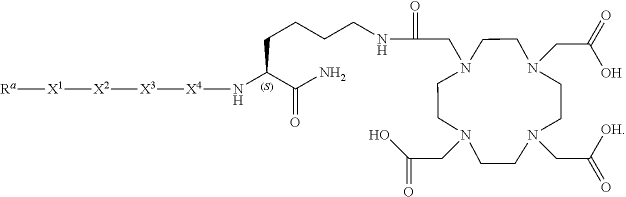

In some embodiments, the compound of Formula (Ib) has the following structure:

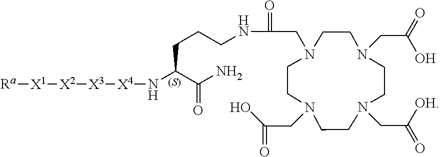

In some embodiments, the compound of Formula (Ib) has the following structure:

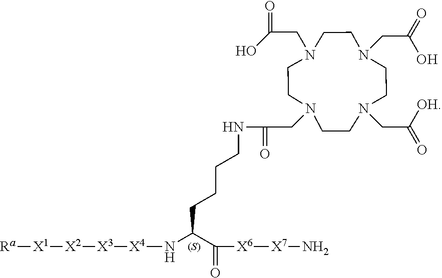

In some embodiments, the compound of Formula (Ib) has the following structure:

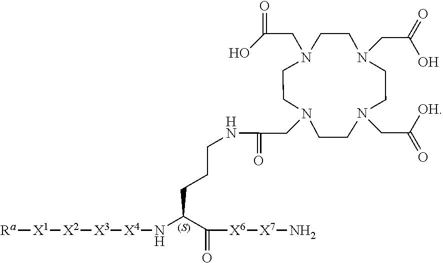

In some embodiments, the compound of Formula (Ib) has the following structure:

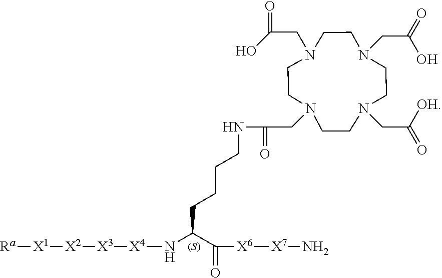

In some embodiments, the compound of Formula (Ib) has the following structure:

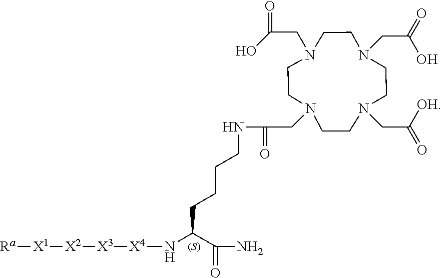

In some embodiments, the compound of Formula (Ib) has the following structure:

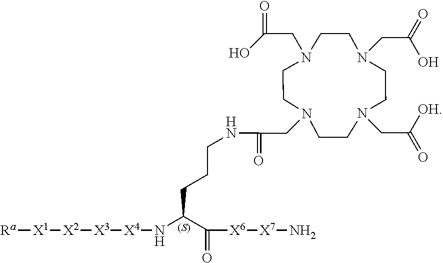

In some embodiments, the compound of Formula (Ib) has the following structure:

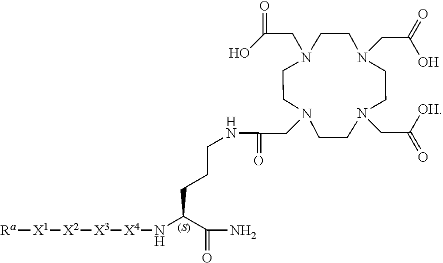

In some embodiments, the compound of Formula (Ib) has one of the following structures:

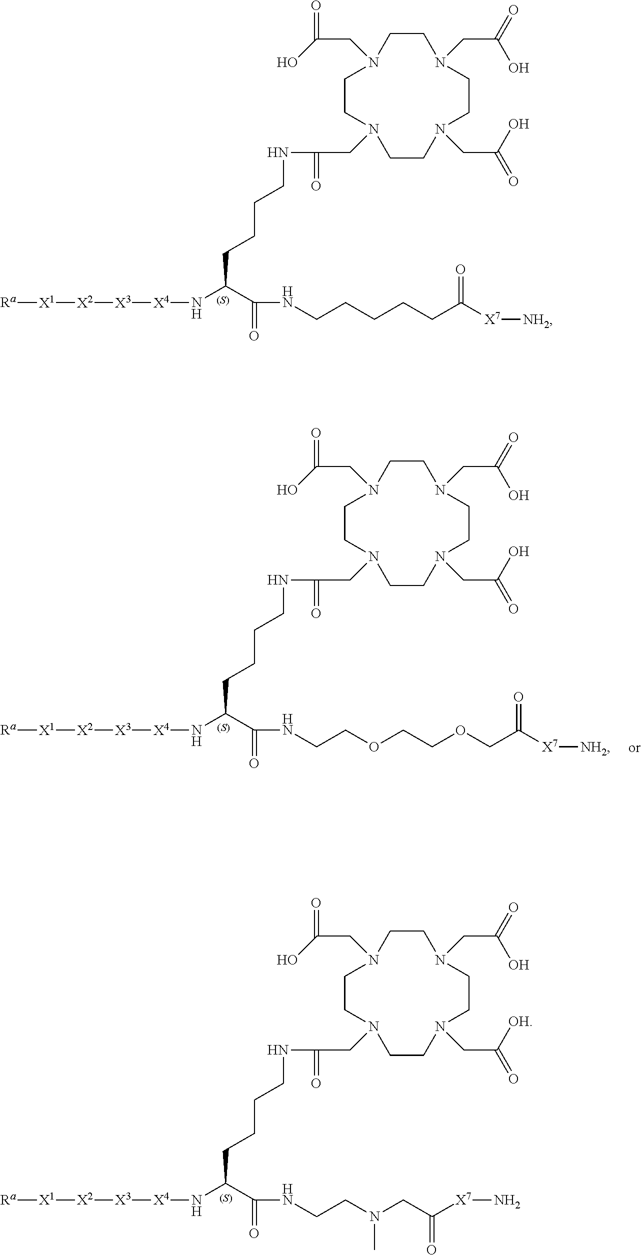

In some embodiments, the compound of Formula (Ib) has the following structure:

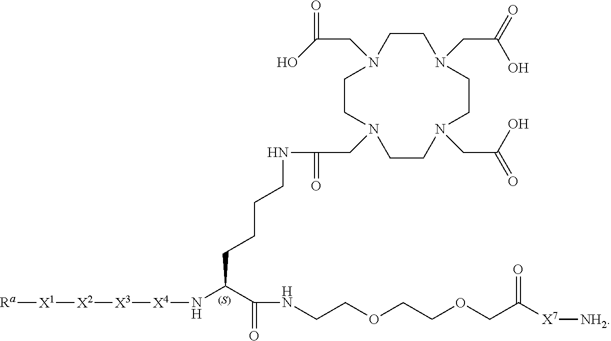

In some embodiments, the compound of Formula (Ib) has one of the following structures:

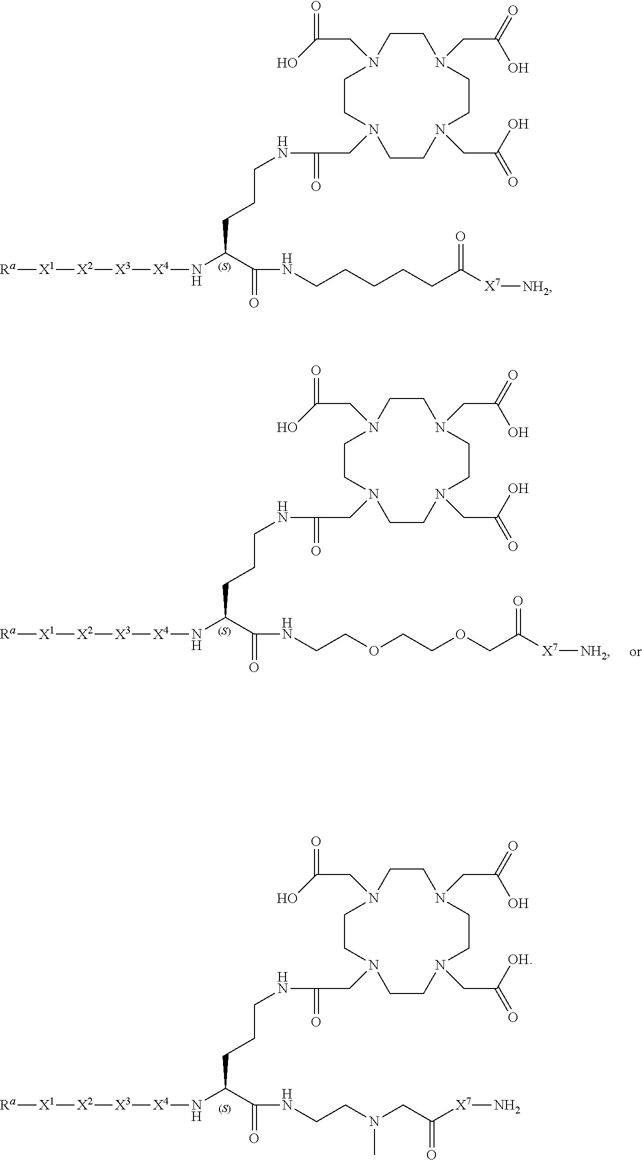

In some embodiments, the compound of Formula (Ib) has the following structure:

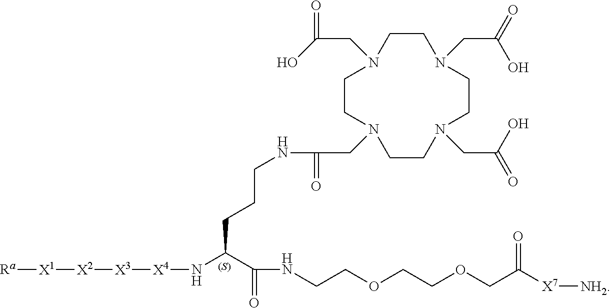

In some embodiments, the compound of Formula (Ic) has the following structure:

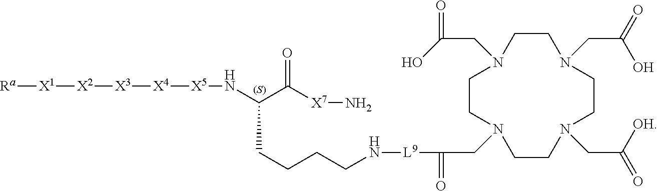

In some embodiments, the compound of Formula (Ic) has the following structure:

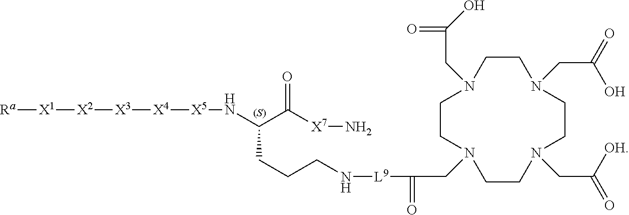

In some embodiments, the compound of Formula (Ic) has the following structure:

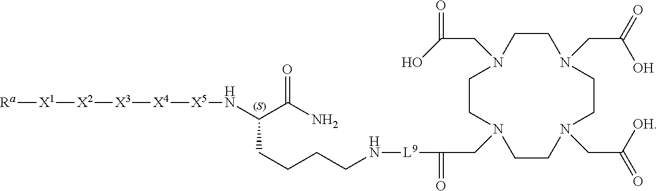

In some embodiments, the compound of Formula (Ic) has the following structure:

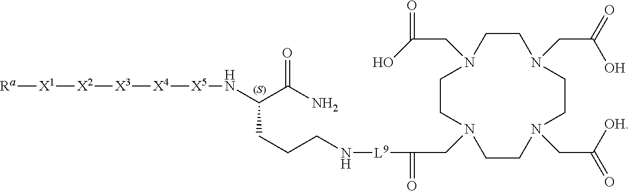

In some embodiments, the compound of Formula (Ic) has the following structure:

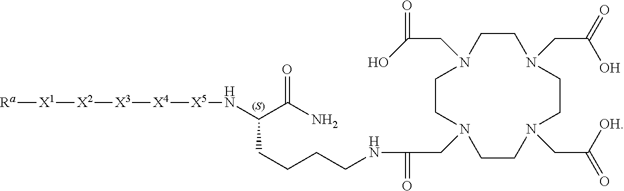

In some embodiments, the compound of Formula (Ic) has the following structure:

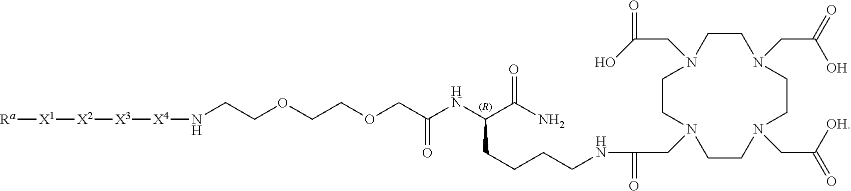

In some embodiments, the compound of Formula (Ic) has the following structure:

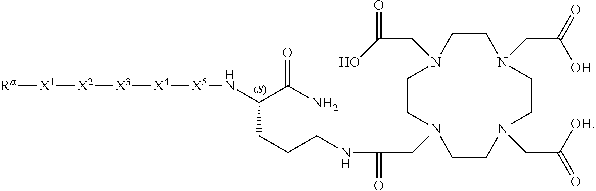

In some embodiments, the compound of Formula (Ic) has the following structure:

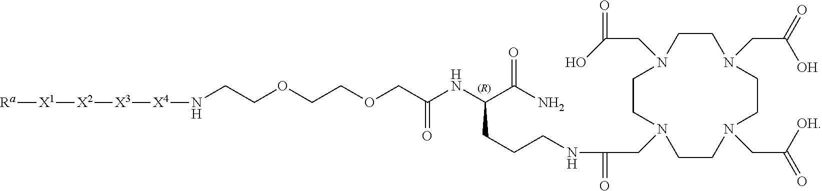

In some embodiments, the compound of Formula (Ic) has the following structure:

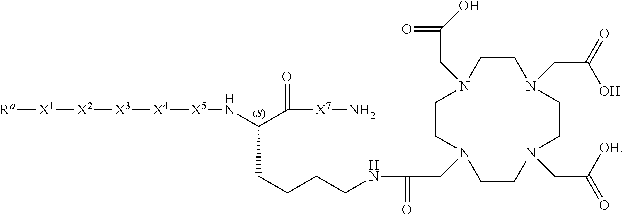

In some embodiments, the compound of Formula (Ic) has the following structure:

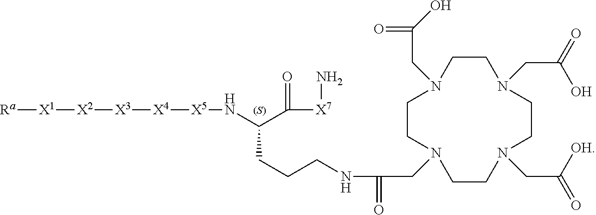

In some embodiments, the compound has the structure of Formula (Id):

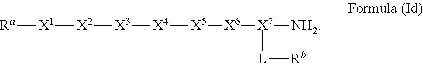

In some embodiments, the compound of Formula (Id) has the following structure:

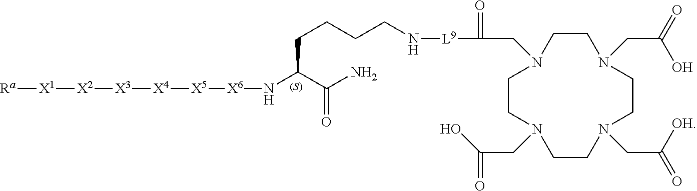

In some embodiments, the compound of Formula (Id) has the following structure:

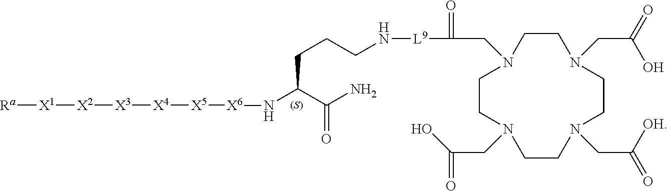

In some embodiments, the compound of Formula (Id) has the following structure:

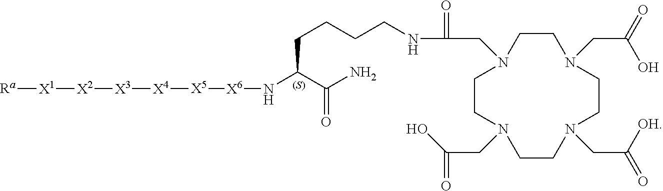

In some embodiments, the compound of Formula (Id) has the following structure:

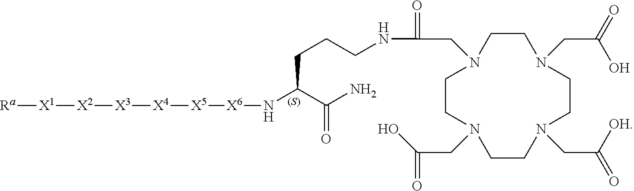

In some embodiments, L⁹ is a bond. In some embodiments, L⁹ is

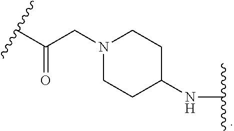 [SMILES: *NC1CCN(CC(*)=O)CC1]

In some embodiments, L⁹ is

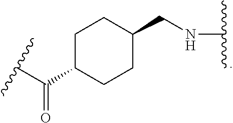 [SMILES: *NC[C@H]1CC[C@H](C(*)=O)CC1]

In some embodiments, L⁹ is

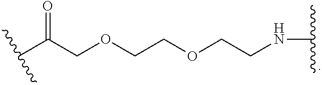 [SMILES: *NCCOCCOCC(*)=O]

In some embodiments, L⁹ is

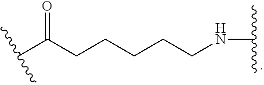 [SMILES: *NCCCCCC(*)=O]

In some embodiments, X¹ is

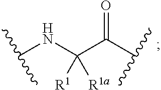

R¹ is -L¹-R^{1b}; L¹ is absent or —CH₂—; R^{1b} is cyclopentyl, cyclohexyl, cycloheptyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, 2-chlorophenyl, 3-chlorophenyl, 4-chlorophenyl, 2,4-dichlorophenyl, 3,4-dichlorophenyl, or 2,5-dichlorophenyl; and R^{1a} is hydrogen or methyl.

In some embodiments, X¹ is

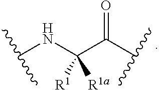

In some embodiments, X¹ is

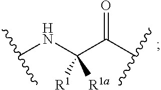

R¹ is -L¹-R^{1b}; L¹ is absent or —CH₂—; R^{1b} is cyclopentyl, cyclohexyl, cycloheptyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, 2-chlorophenyl, 3-chlorophenyl, 4-chlorophenyl, 2,4-dichlorophenyl, 3,4-dichlorophenyl, or 2,5-dichlorophenyl; and R^{1a} is hydrogen or methyl.

In some embodiments, X¹ is

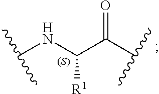

R¹ is -L¹-R^{1b}; L¹ is —CH₂—; and R^{1b} is cyclopentyl, cyclohexyl, cycloheptyl, cyclooctyl, phenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, pyridin-4-yl, 2,3-dihydro-1H-inden-2-yl, 3-cyclopropylphenyl, or [1,1′-biphenyl]-4-yl.

In some embodiments, R^{1b} is cyclohexyl or cycloheptyl. In some embodiments, R¹¹ is cyclohexyl. In some embodiments, R^{1b} is cycloheptyl.

In some embodiments, R^{1b} is phenyl, 4-fluorophenyl, 2,4-difluorophenyl, 3,4-difluorophenyl, 2,5-difluorophenyl, 3,4,5-trifluorophenyl, or perfluorophenyl.

In some embodiments, R^{1b} is cyclopropyl. In some embodiments, R^{1b} is cyclobutyl. In some embodiments, R^{1b} is cyclopentyl. In some embodiments, R^{1b} is cyclohexyl. In some embodiments, R^{1b} is 3-fluorocyclohexyl. In some embodiments, R^{1b} is 4-fluorocyclohexyl. In some embodiments, R^{1b} is 3,3-difluorocyclohexyl, 3,3-dimethylcyclohexyl. In some embodiments, R^{1b} is 4,4-difluorocyclohexyl. In some embodiments, R^{1b} is 4,4-dimethylcyclohexyl. In some embodiments, R^{1b} is cycloheptyl. In some embodiments, R^{1b} is cyclooctyl. In some embodiments, R^{1b} is phenyl. In some embodiments, R^{1b} is 2-fluorophenyl. In some embodiments, R^{1b} is 3-fluorophenyl. In some embodiments, R^{1b} is 4-fluorophenyl. In some embodiments, R^{1b} is 2,4-difluorophenyl. In some embodiments, R^{1b} is 3,4-difluorophenyl. In some embodiments, R^{1b} is 2,5-difluorophenyl. In some embodiments, R^{1b} is 3,4,5-trifluorophenyl. In some embodiments, R^{1b} is perfluorophenyl. In some embodiments, R^{1b} is 2-chlorophenyl. In some embodiments, R^{1b} is 3-chlorophenyl. In some embodiments, R^{1b} is 4-chlorophenyl. In some embodiments, R^{1b} is 2,4-dichlorophenyl. In some embodiments, R^{1b} is 3,4-dichlorophenyl. In some embodiments, R^{1b} is 2,5-dichlorophenyl. In some embodiments, R^{1b} is 3,4,5-trichlorophenyl. In some embodiments, R^{1b} is 4-bromophenyl. In some embodiments, R^{1b} is 4-methylphenyl. In some embodiments, R^{1b} is 4-trifluoromethylphenyl. In some embodiments, R^{1b} is 3,4-bis(trifluoromethyl)phenyl. In some embodiments, R^{1b} is 4-vinylphenyl. In some embodiments, R^{1b} is 4-tert-butylphenyl. In some embodiments, R^{1b} is 2-hydroxyphenyl. In some embodiments, R^{1b} is 4-hydroxyphenyl. In some embodiments, R^{1b} is 4-methoxyphenyl. In some embodiments, R^{1b} is pyridin-2-yl. In some embodiments, R^{1b} is pyridin-3-yl. In some embodiments, R^{1b} is pyridin-4-yl. In some embodiments, R^{1b} is 4-aminophenyl. In some embodiments, R^{1b} is 4-carbamoylphenyl. In some embodiments, R^{1b} is 4-ureidophenyl. In some embodiments, R^{1b} is 4-acetylphenyl. In some embodiments, R^{1b} is 4-azidophenyl. In some embodiments, R^{1b} is 4-(azidomethyl)phenyl. In some embodiments, R^{1b} is 2,3-dihydro-1H-inden-1-yl. In some embodiments, R^{1b} is 2,3-dihydro-1H-inden-2-yl. In some embodiments, R^{1b} is 3-cyclopropylphenyl. In some embodiments, R^{1b} is [1,1′-biphenyl]-4-yl. In some embodiments, R^{1b} is piperidin-1-yl. In some embodiments, R^{1b} is 1,2,3,4-tetrahydronaphthalen-1-yl. In some embodiments, R^{1b} is 5,6,7,8-tetrahydronaphthalen-2-yl. In some embodiments, R^{1b} is 1H-indol-3-yl. In some embodiments, R^{1b} is 2,3-dihydrobenzofuran-6-yl. In some embodiments, R^{1b} is naphthalen-1-yl. In some embodiments, R^{1b} is adamantan-1-yl. In the aforementioned embodiments, each R^{1b} is optionally additionally substituted with 1 or more —F, —Br, —Cl, —I, —CF₃, or —C₁-C₆ alkyl. In some embodiments, R¹ and R^{1a} are taken together with the carbon atoms to which they are attached to form:

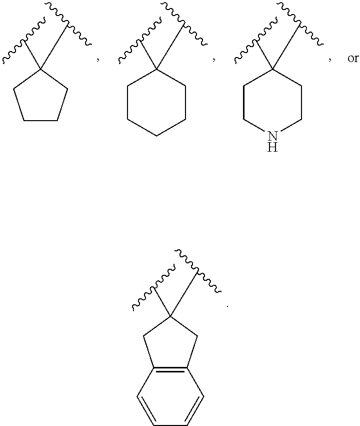 [SMILES: *C1(*)CCCC1 ; *C1(*)CCCCC1 ; *C1(*)CCNCC1 ; *C1(*)Cc2ccccc2C1]

In some embodiments, X¹ is

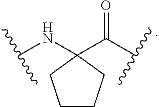 [SMILES: *NC1(C(*)=O)CCCC1]

In some embodiments, X¹ is

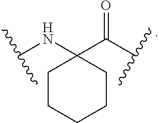 [SMILES: *NC1(C(*)=O)CCCCC1]

In some embodiments, X¹ is

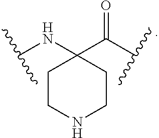 [SMILES: *NC1(C(*)=O)CCNCC1]

In some embodiments, X¹ is

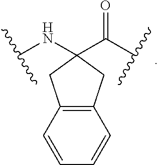 [SMILES: *NC1(C(*)=O)Cc2ccccc2C1]

In some embodiments, X¹ is

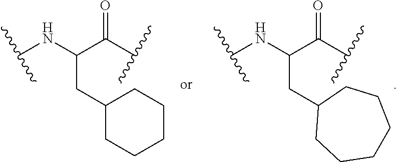 [SMILES: *NC(CC1CCCCC1)C(*)=O ; *NC(CC1CCCCCC1)C(*)=O]

In some embodiments, X¹ is

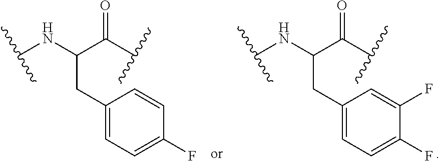 [SMILES: *NC(Cc1ccc(F)cc1)C(*)=O ; *NC(Cc1ccc(F)c(F)c1)C(*)=O]

In some embodiments, X² is

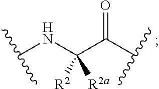

R² is -L²-R^{2b}; L² is absent or —CH₂—; R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl, or 4′-methyl-[1,1′-biphenyl]-4-yl; and R^{2a} is hydrogen or methyl. In some embodiments, R^{2b} is cyclohexyl, cycloheptyl, 2,3-dihydro-1H-inden-2-yl or [1,1-biphenyl]-4-yl.

In some embodiments, X² is

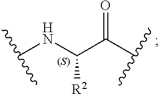

R² is -L²-R^{2b}; L² is absent; and R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl, or 4′-methyl-[1,1′-biphenyl]-4-yl. In some embodiments, R^{2b} is cyclohexyl or cycloheptyl.

In some embodiments, X² is

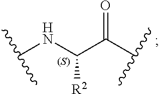

R² is -L²-R^{2b}; L² is —CH₂—; and R^{2b} is 2,3-dihydro-1H-inden-2-yl or [1,1′-biphenyl]-4-yl. In some embodiments, X² is

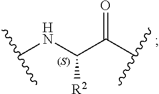

R² is -L²-R^{2b}; L² is absent; and R^{2b} is 2,3-dihydro-1H-inden-2-yl or [1,1′-biphenyl]-4-yl.

In some embodiments, R^{2b} is methyl. In some embodiments, R^{2b} is pent-3-yl. In some embodiments, R^{2b} is tert-butyl. In some embodiments, R^{2b} is 1,1,1,3,3,3-hexafluoropropan-2-yl. In some embodiments, R^{2b} is cyclopropyl. In some embodiments, R^{2b} is cyclobutyl. In some embodiments, R^{2b} is cyclopentyl. In some embodiments, R^{2b} is cyclohexyl. In some embodiments, R^{2b} is 3-fluorocyclohexyl. In some embodiments, R^{2b} is 4-fluorocyclohexyl. In some embodiments, R^{2b} is 3,3-difluorocyclohexyl. In some embodiments, R^{2b} is 3,3-dimethylcyclohexyl. In some embodiments, R^{2b} is 4,4-difluorocyclohexyl. In some embodiments, R^{2b} is 4,4-dimethylcyclohexyl. In some embodiments, R^{2b} is cycloheptyl. In some embodiments, R^{2b} is cyclooctyl. In some embodiments, R^{2b} is phenyl. In some embodiments, R²¹ is 2-fluorophenyl. In some embodiments, R^{2b} is 3-fluorophenyl. In some embodiments, R^{2b} is 4-fluorophenyl. In some embodiments, R² is 2,4-difluorophenyl. In some embodiments, R^{2b} is 3,4-difluorophenyl. In some embodiments, R^{2b} is 2,5-difluorophenyl. In some embodiments. R^{2b} is 3,4,5-trifluorophenyl. In some embodiments, R^{2b} is perfluorophenyl. In some embodiments, R^{2b} is 2-chlorophenyl. In some embodiments, R^{2b} is 3-chlorophenyl. In some embodiments, R^{2b} is 4-chlorophenyl. In some embodiments, R^{2b} is 2,4-dichlorophenyl. In some embodiments, R^{2b} is 3,4-dichlorophenyl. In some embodiments, R^{2b} is 2,5-dichlorophenyl. In some embodiments, R^{2b} is 3,4,5-trichlorophenyl. In some embodiments, R²⁶ is 4-bromophenyl. In some embodiments, R^{2b} is 4-methylphenyl. In some embodiments, R^{2b} is 4-trifluoromethylphenyl. In some embodiments, R^{2b} is 3,4-bis(trifluoromethyl)phenyl. In some embodiments, R^{2b} is 4-vinylphenyl. In some embodiments, R^{2b} is 4-tert-butylphenyl. In some embodiments, R^{2b} is 2-hydroxyphenyl. In some embodiments, R^{2b} is 4-hydroxyphenyl. In some embodiments, R^{2b} is 4-methoxyphenyl. In some embodiments, R^{2b} is 4-aminophenyl. In some embodiments, R^{2b} is 4-carbamoylphenyl. In some embodiments, R^{2b} is 4-ureidophenyl. In some embodiments, R^{2b} is 4-acetylphenyl. In some embodiments, R^{2b} is 4-azidophenyl. In some embodiments, R^{2b} is 4-(azidomethyl)phenyl. In some embodiments, R^{2b} is pyridin-2-yl. In some embodiments, R^{2b} is pyridin-3-yl. In some embodiments, R^{2b} is pyridin-4-yl. In some embodiments, R^{2b} is 2,3-dihydro-1H-inden-1-yl. In some embodiments, R^{2b} is 2,3-dihydro-1H-inden-2-yl. In some embodiments, R^{2b} is 3-cyclopropylphenyl. In some embodiments, R^{2b} is [1,1′-biphenyl]-4-yl. In some embodiments, R^{2b} is 4′-fluoro-[1,1′-biphenyl. In some embodiments, R^{2b} is 4′-methyl-[1,1′-biphenyl]-4-yl. In some embodiments, R^{2b} is 1,2,3,4-tetrahydronaphthalen-1-yl. In some embodiments, R^{2b} is 5,6,7,8-tetrahydronaphthalen-2-yl. In some embodiments, R^{2b} is 1H-indol-3-yl. In some embodiments, R^{2b} is 2,3-dihydrobenzofuran-6-yl. In some embodiments, R^{2b} is naphthalen-1-yl. In some embodiments, R^{2b} is adamantan-1-yl. In the aforementioned embodiments, each R^{2b} is optionally additionally substituted with 1 or more —F, —Br, —Cl, —I, —CF₃, or —C₁-C₆ alkyl.

In some embodiments, R² and R^{2a} are taken together with the carbon atoms to which they are attached to form:

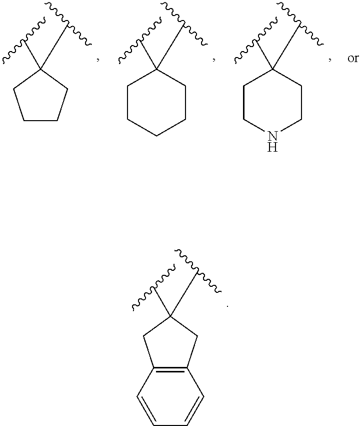 [SMILES: *C1(*)CCCC1 ; *C1(*)CCCCC1 ; *C1(*)CCNCC1 ; *C1(*)Cc2ccccc2C1]

In some embodiments, X² is

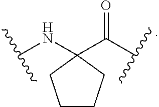 [SMILES: *NC1(C(*)=O)CCCC1]

In some embodiments, X² is

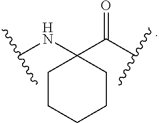 [SMILES: *NC1(C(*)=O)CCCCC1]

In some embodiments, X² is

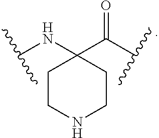 [SMILES: *NC1(C(*)=O)CCNCC1]

In some embodiments, X² is

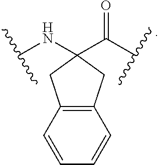 [SMILES: *NC1(C(*)=O)Cc2ccccc2C1]

In some embodiments, X² is

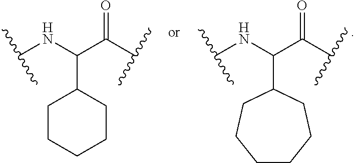 [SMILES: *NC(C(*)=O)C1CCCCC1 ; *NC(C(*)=O)C1CCCCCC1]

In some embodiments, L¹ is absent. In some embodiments, L¹ is —CH₂—. In some embodiments, L¹ is —CHPh-.

In some embodiments, L² is absent. In some embodiments, L² is —CH₂—. In some embodiments, L² is —CHPh-.

In some embodiments, X³ is glycine, (S)-alanine (Ala), or norleucine (Nle). In some embodiments, X³ is (S)-alanine (Ala) or (S)-norleucine (Nle). In some embodiments, X³ is (S)-alanine (Ala). In some embodiments, X³ is (S)-norleucine (Nle).

In some embodiments, X³ is glycine. In some embodiments, X³ is alanine (Ala). In some embodiments, X³ is (S)-alanine (Ala). In some embodiments, X³ is (S)-trifluoromethylalanine. In some embodiments, X³ is (S)-homoalanine. In some embodiments, X³ is (S)-Glu. In some embodiments, X³ is (S)-Gln. In some embodiments, X³ is (S)-Orn. In some embodiments, X³ is (S)-lysine (Lys). In some embodiments, X³ is (S)-serine (Ser). In some embodiments, X³ is (S)—N-methylalanine (N-Me-Ala). In some embodiments, 3 is 2-amino-2-methylpropanoic acid (AIB). In some embodiments, X³ is 1-aminocyclopropane-1-carboxylic acid (ACC). In some embodiments, X³ is norleucine (Nle). In some embodiments, X³ is (S)-3-aminoalanine. In some embodiments, X³ is (S)-2-amino-6-azidohexanoic acid. In some embodiments, X³ is (S)-2-amino-6-(4-hexyl-1H-1,2,3-triazol-1-yl)hexanoic acid. In some embodiments, X³ is (S)-2-amino-6-(4-((2-methoxyethoxy)methyl)-1H-1,2,3-triazol-1-yl)hexanoic acid. In some embodiments, X³ is (S)-2-amino-6-(4-(cyclopentylmethyl)-1H-1,2,3-triazol-1-yl)hexanoic acid. In some embodiments, X³ is (S)-2-amino-6-(4-benzyl-1H-1,2,3-triazol-1-yl)hexanoic acid. In some embodiments, X³ is (S)-proline (Pro). In some embodiments, X³ is (S)-cyclopropylglycine. In some embodiments, X³ is (S)-biphenylalanine (Bip).

In some embodiments, X⁴ is (S)-arginine (Arg), (S)-ornithine (Orn), (S)-phenylalanine (Phe), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-(pyridin-4-yl)alanine (4-Pal), (S)-4-aminophenylalanine (Phe(4-NH₂)), (S)-3-(6-aminopyridin-3-yl)alanine ((3-Pal(6-NH₂)), or (S)-2-((2-aminopyridin-4-yl)-λ²-azaneyl)ethyl alanine. In some embodiments, X⁴ is (S)-arginine (Arg), or (S)-ornithine (Orn). In some embodiments, X⁴ is (S)-arginine (Arg), (S)-ornithine (Orn), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-3-(6-aminopyridin-3-yl)alanine ((3-Pal(6-NH₂)), or (S)-2-((2-aminopyridin-4-yl)-λ²-azaneyl)ethyl alanine. In some embodiments, X⁴ is (S)-2-amino-3-(4-aminophenyl)propanoic acid (Phe(4-NH₂)), (S)-2-amino-3-(6-aminopyridin-3-yl)propanoic acid, or (S)-2-((2-aminopyridin-4-yl)-λ²-azaneyl)ethyl alanine.

In some embodiments, X⁴ is (S)-arginine (Arg). In some embodiments, X⁴ is (S)-3-amino-6-guanidinohexanoic acid. In some embodiments, X⁴ is N6-carbamimidoyl-(S)-lysine. In some embodiments, X⁴ is (S)-2-amino-5-ureidopentanoic acid. In some embodiments, X⁴ is (S)-homoarginine. In some embodiments, X⁴ is N2-methyl-(S)-arginine. In some embodiments, X⁴ is N^{ω}-methyl-(S)-arginine. In some embodiments, X⁴ is N^{ω}-nitro-(S)-arginine. In some embodiments, X⁴ is (E)-N^{ω},N^{ω}′-dimethyl-(S)-arginine. In some embodiments, X⁴ is (E)-N^{ω},N^{ω}′-diethyl-(S)-arginine. In some embodiments, X⁴ is (S)-ornithine (Orn). In some embodiments, X⁴ is (S)-lysine. In some embodiments, X⁴ is (S)-phenylalanine (Phe). In some embodiments, X⁴ is (S)-cyclohexylglycine (Chg). In some embodiments, X⁴ is (S)-cyclohexylalanine (Cha). In some embodiments, X⁴ is norleucine (Nle). In some embodiments, X⁴ is (S)-3-amino-alanine (H-Dap-OH). In some embodiments, X⁴ is piperidinyl-1-yl-(S)-alanine. In some embodiments, X⁴ is N⁴,N⁴-dimethyl-(S)-asparagine (N⁴,N⁴-dimethyl-Asn). In some embodiments, X⁴ is (S)-2-(piperidin-4-yl)-glycine (Gly(piperidin-4-yl)). In some embodiments, X⁴ is (S)-3-pyridin-3-yl-beta-alanine (H-βAla(3-pyrid-3-yl)-OH). In some embodiments, X⁴ is (S)-2-(1-carbamimidoylpiperidin-4-yl)-glycine (Gly(1-carbamimidoylpiperidin-4-yl)). In some embodiments, X⁴ is (S)-3-amino-3-(1-carbamimidoylpiperidin-3-yl)propanoic acid. In some embodiments, X⁴ is (S)-phenylglycine (Phg). In some embodiments, X⁴ is (S)-4-fluorophenylalanine (Phe(4-F)). In some embodiments, X⁴ is (S)-4-aminophenylalanine (Phe(4-NH₂)). In some embodiments, X⁴ is (S)-4-nitrophenylalanine (Phe(4-NO₂)). In some embodiments, X⁴ is (S)-4-carbamoylphenylalanine. In some embodiments, X⁴ is (S)-4-guanidinophenylalanine. In some embodiments, X⁴ is (S)-asparagine (Asn). In some embodiments, X⁴ is (S)-glutamic acid (Glu). In some embodiments, X⁴ is (S)-proline. In some embodiments, X⁴ is (S)-4-ureidophenylalanine. In some embodiments, X⁴ is (S)-(2-aminoethoxy)phenylalanine (Phe(4-OCH₂CH₂NH₂)). In some embodiments, X⁴ is (S)-(pyridin-4-yl)alanine (3-Pal). In some embodiments, X⁴ is (S)-(pyridin-4-yl)alanine (4-Pal). In some embodiments, X⁴ is (S)-4-aminophenylalanine (Phe(4-NH₂)). In some embodiments, X⁴ is (S)-3-(6-aminopyridin-3-yl)alanine ((3-Pal(6-N1H₂)). In some embodiments, X⁴ is (S)-2-((2-aminopyridin-4-yl)-λ²-azaneyl)ethyl alanine. In some embodiments, X⁴ is N6-nicotinoyl-Lys. In some embodiments, X⁴ is 2-oxo-2-(piperazin-1-yl)ethyl alanine. In some embodiments, X⁴ is (S)-2-(piperidin-4-yl)ethylalanine. In some embodiments, X⁴ is (S)-3-((piperazin-1-yl)propyl)-glycine. In some embodiments, X⁴ is (S)-3-((4-methylpiperazin-1-yl)propyl)-glycine. In some embodiments, X⁴ is (S)-3-(4-benzylpiperidin-1-yl)propylglycine. In some embodiments, X⁴ is (S)-3-((4-benzylpiperazin-1-yl)propyl)-glycine. In some embodiments, X⁴ is N6-(1-benzylpiperidine-4-carbonyl)-Lys. In some embodiments, X⁴ is (S)-3-((3,4-dihydroisoquinolin-2(1H)-yl)propyl)-glycine. In some embodiments, X⁴ is (S)-2-pyridin-4-ylthynyl alanine. In some embodiments, X⁴ is (S)-(4-(aminomethyl)-1H-1,2λ²,3λ²-triazol-1-yl)methyl)-glycine. In some embodiments, X⁴ is (S)-(4-(piperazin-1-ylmethyl)-1H-1,2λ²,3λ²-triazol-1-yl)methyl)-glycine. In some embodiments, X⁴ is (S)-2-((1H-imidazol-4-yl)ethyl)glycine. In some embodiments, X⁴ is (4-((R)-2,6-dioxohexahydropyrimidine-4-carboxamido)phenyl)alanine.

In some embodiments, X⁴ is (S)-4-methylpiperazine-2-carboxylic acid.

In some embodiments, X⁴ is

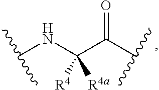

where R⁴ and R^{4a} are taken together with the carbon atom to which they are attached to form

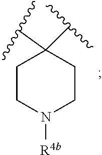

wherein R^{4b} is H, —C(═NH)N—H₂, —C(═O)CH(CH₃)NCH₃, —CH₂C(═O)NHC₁-C₁₀ alkyl-NH₂, or —C(═O)CH₂NHC₁-C₁₀ alkyl. In some embodiments X⁴ is

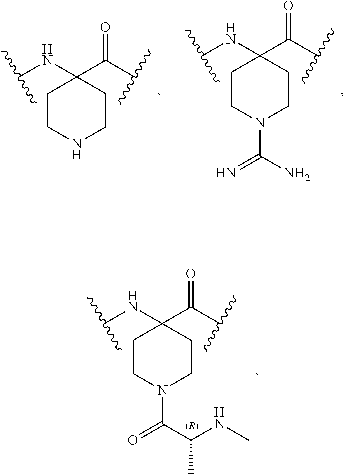

or

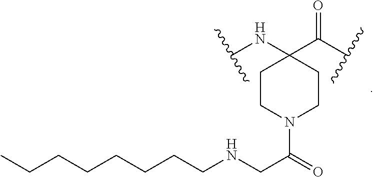 [SMILES: *NC1(C(*)=O)CCN(C(=O)CNCCCCCCCC)CC1]

In some embodiments, X⁴ is

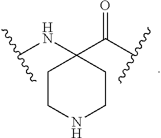 [SMILES: *NC1(C(*)=O)CCNCC1]

In some embodiments, X⁴ is

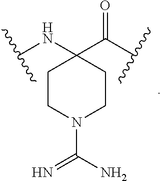 [SMILES: *NC1(C(*)=O)CCN(C(=N)N)CC1]

In some embodiments, X⁴ is

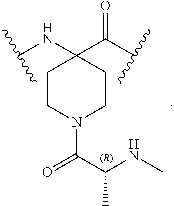

In some embodiments, X⁴ is

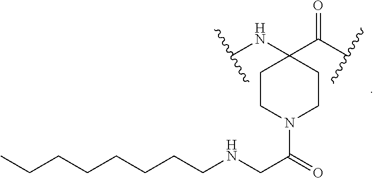 [SMILES: *NC1(C(*)=O)CCN(C(=O)CNCCCCCCCC)CC1]

In some embodiments, X⁴ is

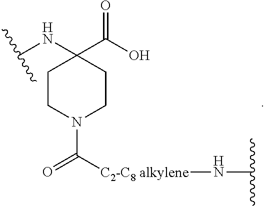

In some embodiments, X⁴ is

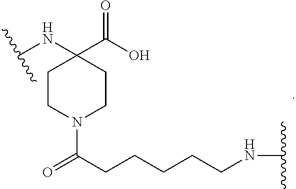 [SMILES: *NCCCCCC(=O)N1CCC(N*)(C(=O)O)CC1]

In some embodiments, X⁴ is

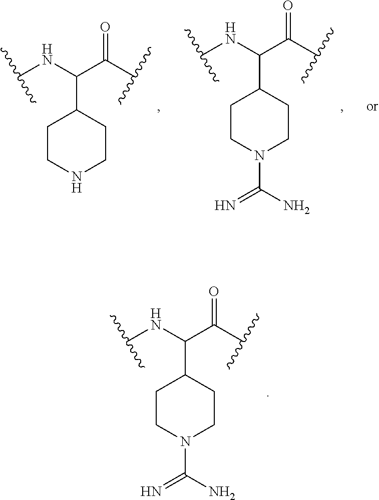 [SMILES: *NC(C(*)=O)C1CCNCC1 ; *NC(C(*)=O)C1CCN(C(=N)N)CC1 ; *NC(C(*)=O)C1CCN(C(=N)N)CC1]

In some embodiments, X⁴ is

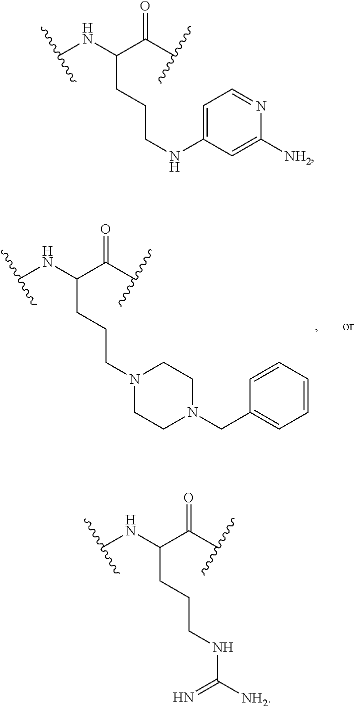 [SMILES: *NC(CCCNc1ccnc(N)c1)C(*)=O ; *NC(CCCN1CCN(Cc2ccccc2)CC1)C(*)=O ; *NC(CCCNC(=N)N)C(*)=O]

In some embodiments, —X¹—X²—X³—X⁴— is:

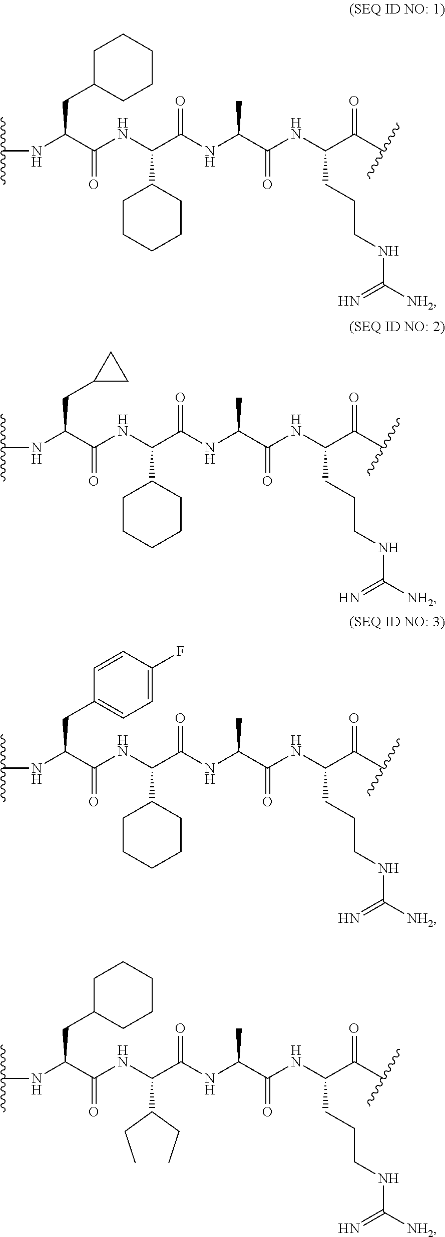 [SMILES: *N[C@@H](CC1CCCCC1)C(=O)N[C@H](C(=O)N[C@@H](C)C(=O)N[C@@H](CCCNC(=N)N)C(*)=O)C1CCCCC1 ; *N[C@@H](CC1CC1)C(=O)N[C@H](C(=O)N[C@@H](C)C(=O)N[C@@H](CCCNC(=N)N)C(*)=O)C1CCCCC1 ; *N[C@@H](Cc1ccc(F)cc1)C(=O)N[C@H](C(=O)N[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N[C@@H](CCCNC(=N)N)C(*)=O)C(CC)CC]

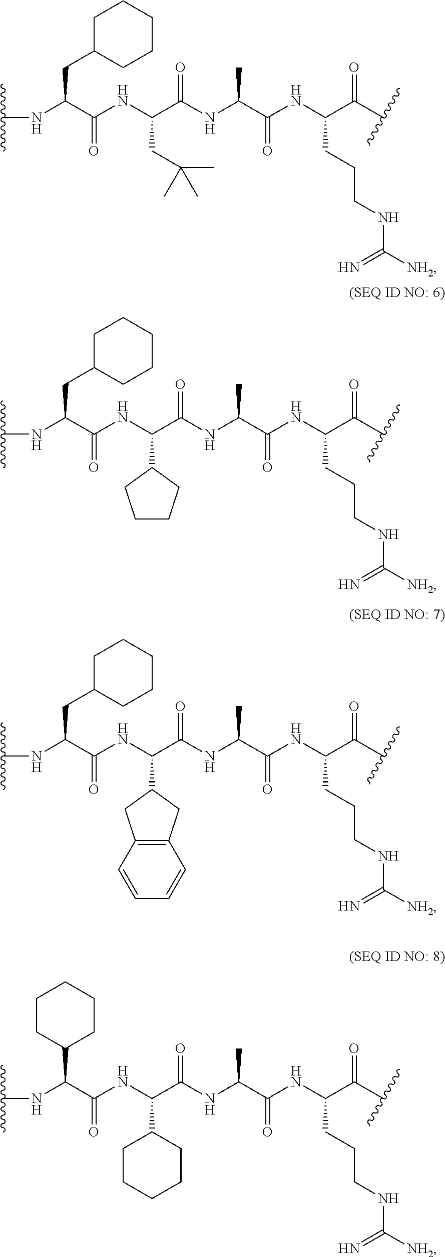 [SMILES: *N[C@@H](CC1CCCCC1)C(=O)N[C@@H](CC(C)(C)C)C(=O)N[C@@H](C)C(=O)N[C@@H](CCCNC(=N)N)C(*)=O ; *N[C@@H](CC1CCCCC1)C(=O)N[C@H](C(=O)N[C@@H](C)C(=O)N[C@@H](CCCNC(=N)N)C(*)=O)C1CCCC1 ; *N[C@@H](CC1CCCCC1)C(=O)N[C@H](C(=O)N[C@@H](C)C(=O)N[C@@H](CCCNC(=N)N)C(*)=O)C1Cc2ccccc2C1 ; *N[C@H](C(=O)N[C@H](C(=O)N[C@@H](C)C(=O)N[C@@H](CCCNC(=N)N)C(*)=O)C1CCCCC1)C1CCCCC1]

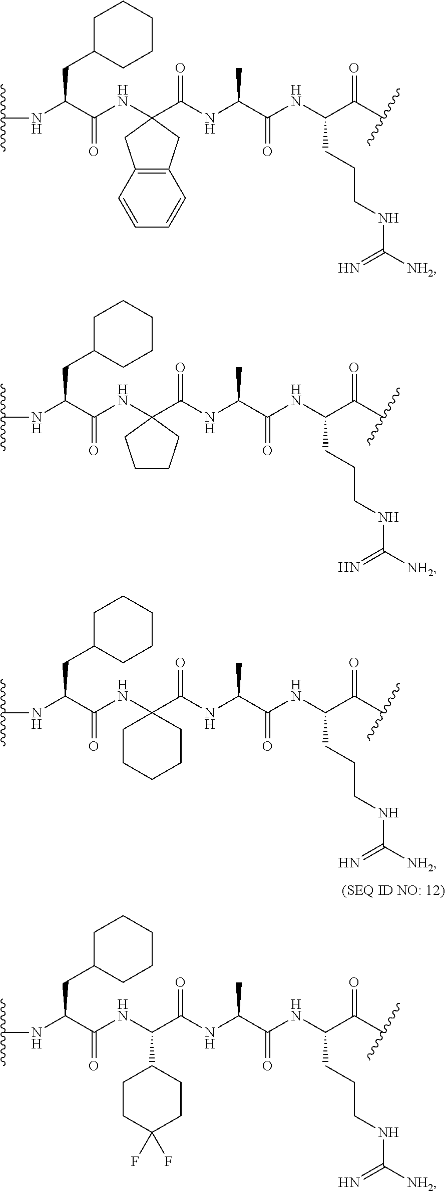 [SMILES: *N[C@@H](CC1CCCCC1)C(=O)NC1(C(=O)N[C@@H](C)C(=O)N[C@@H](CCCNC(=N)N)C(*)=O)Cc2ccccc2C1 ; *N[C@@H](CC1CCCCC1)C(=O)NC1(C(=O)N[C@@H](C)C(=O)N[C@@H](CCCNC(=N)N)C(*)=O)CCCC1 ; *N[C@@H](CC1CCCCC1)C(=O)NC1(C(=O)N[C@@H](C)C(=O)N[C@@H](CCCNC(=N)N)C(*)=O)CCCCC1 ; *N[C@@H](CC1CCCCC1)C(=O)N[C@H](C(=O)N[C@@H](C)C(=O)N[C@@H](CCCNC(=N)N)C(*)=O)C1CCC(F)(F)CC1]

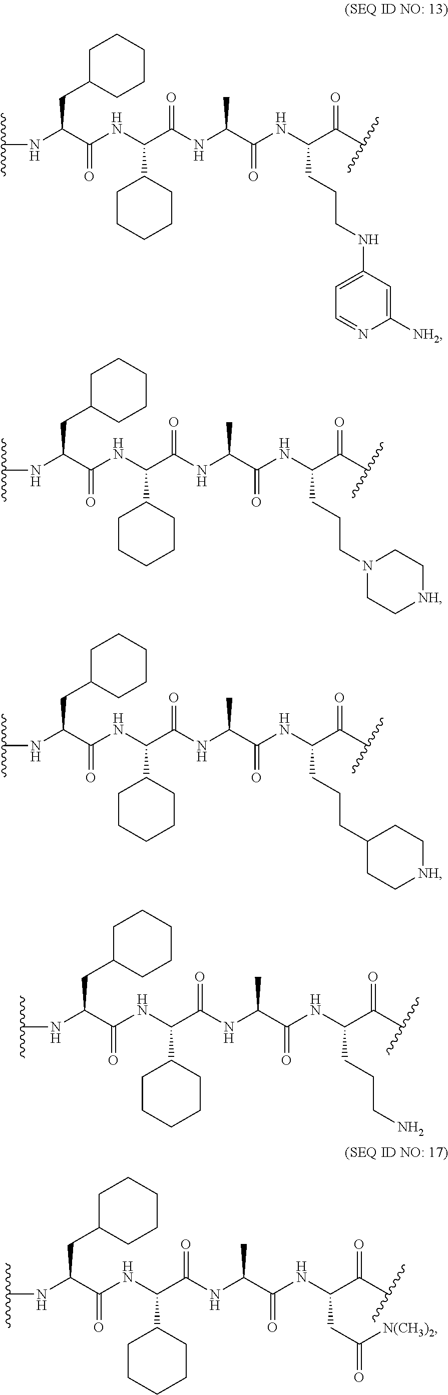 [SMILES: *N[C@@H](CC1CCCCC1)C(=O)N[C@H](C(=O)N[C@@H](C)C(=O)N[C@@H](CCCNc1ccnc(N)c1)C(*)=O)C1CCCCC1 ; *N[C@@H](CC1CCCCC1)C(=O)N[C@H](C(=O)N[C@@H](C)C(=O)N[C@@H](CCCN1CCNCC1)C(*)=O)C1CCCCC1 ; *N[C@@H](CC1CCCCC1)C(=O)N[C@H](C(=O)N[C@@H](C)C(=O)N[C@@H](CCCC1CCNCC1)C(*)=O)C1CCCCC1 ; *N[C@@H](CC1CCCCC1)C(=O)N[C@H](C(=O)N[C@@H](C)C(=O)N[C@@H](CCCN)C(*)=O)C1CCCCC1 ; *N[C@@H](CC1CCCCC1)C(=O)N[C@H](C(=O)N[C@@H](C)C(=O)N[C@@H](CC(=O)N(C)C)C(*)=O)C1CCCCC1]

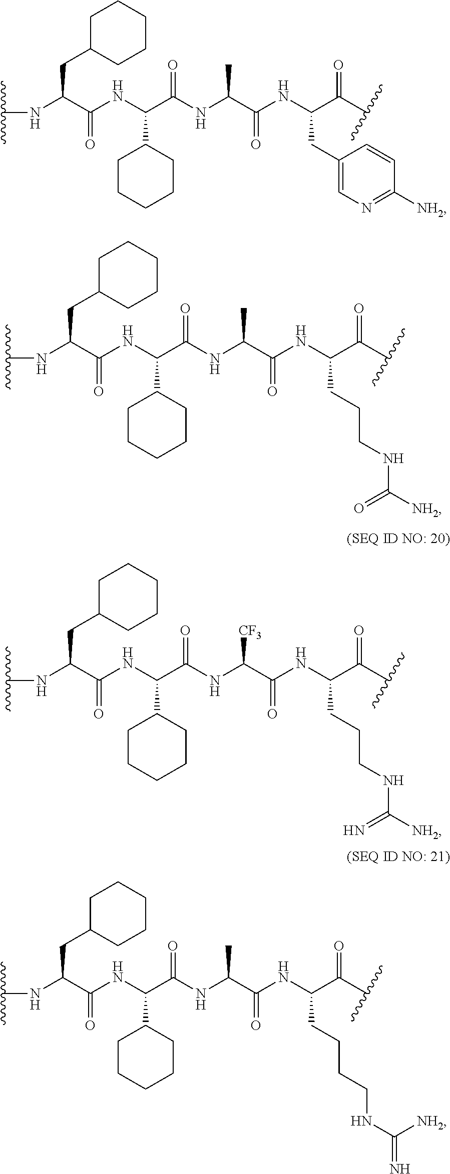

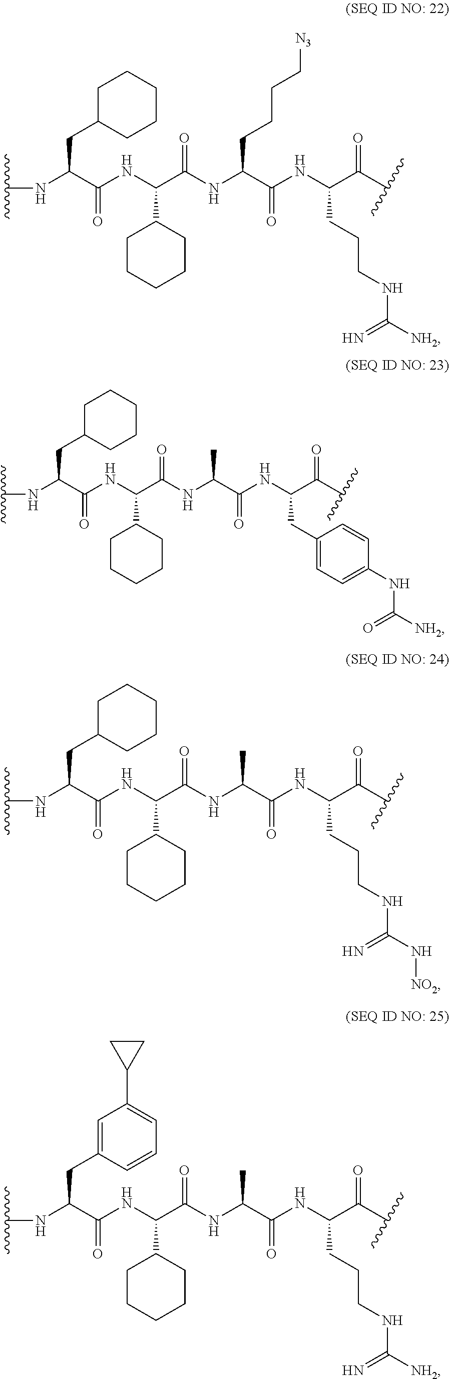 [SMILES: *N[C@@H](CC1CCCCC1)C(=O)N[C@H](C(=O)N[C@@H](CCCCN=[N+]=[N-])C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N[C@@H](Cc1ccc(NC(N)=O)cc1)C(*)=O)C1CCCCC1 ; *N[C@@H](CC1CCCCC1)C(=O)N[C@H](C(=O)N[C@@H](C)C(=O)N[C@@H](CCCNC(=N)N[N+](=O)[O-])C(*)=O)C1CCCCC1 ; *N[C@@H](Cc1cccc(C2CC2)c1)C(=O)N[C@H](C(=O)N[C@@H](C)C(=O)N[C@@H](CCCNC(=N)N)C(*)=O)C1CCCCC1]

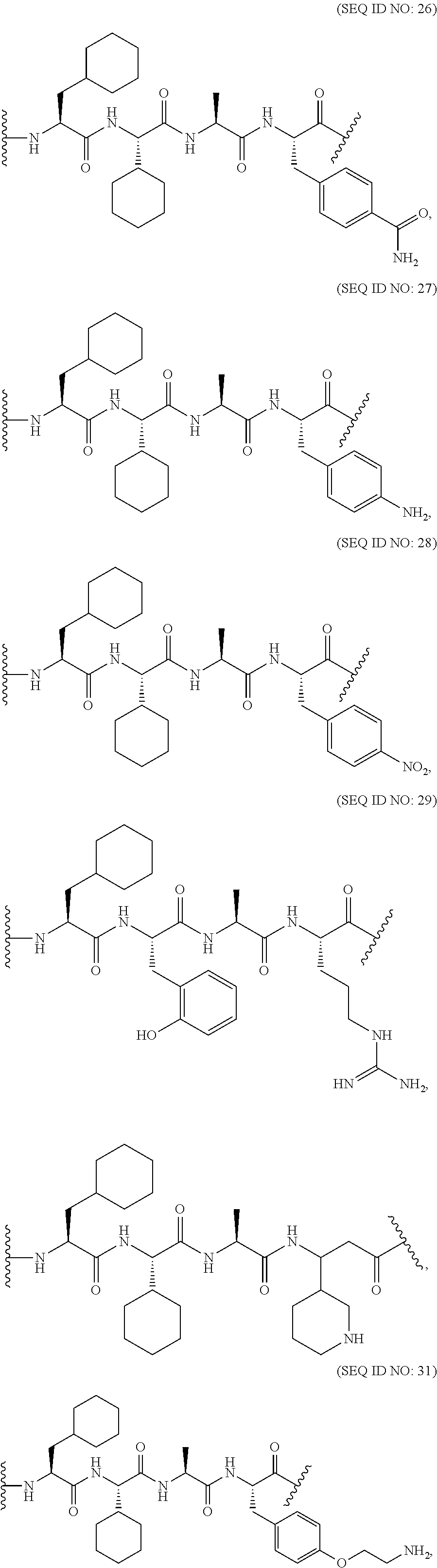

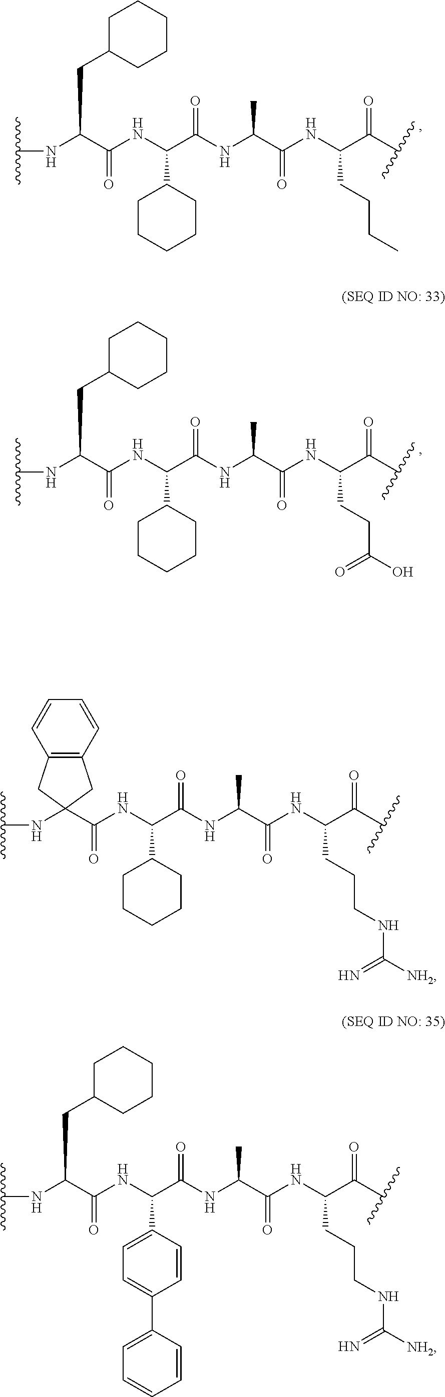 [SMILES: *N[C@@H](CC1CCCCC1)C(=O)N[C@H](C(=O)N[C@@H](C)C(=O)N[C@@H](CCCC)C(*)=O)C1CCCCC1 ; *N[C@@H](CC1CCCCC1)C(=O)N[C@H](C(=O)N[C@@H](C)C(=O)N[C@@H](CCC(=O)O)C(*)=O)C1CCCCC1 ; *NC1(C(=O)N[C@H](C(=O)N[C@@H](C)C(=O)N[C@@H](CCCNC(=N)N)C(*)=O)C2CCCCC2)Cc2ccccc2C1 ; *N[C@@H](CC1CCCCC1)C(=O)N[C@H](C(=O)N[C@@H](C)C(=O)N[C@@H](CCCNC(=N)N)C(*)=O)c1ccc(-c2ccccc2)cc1]

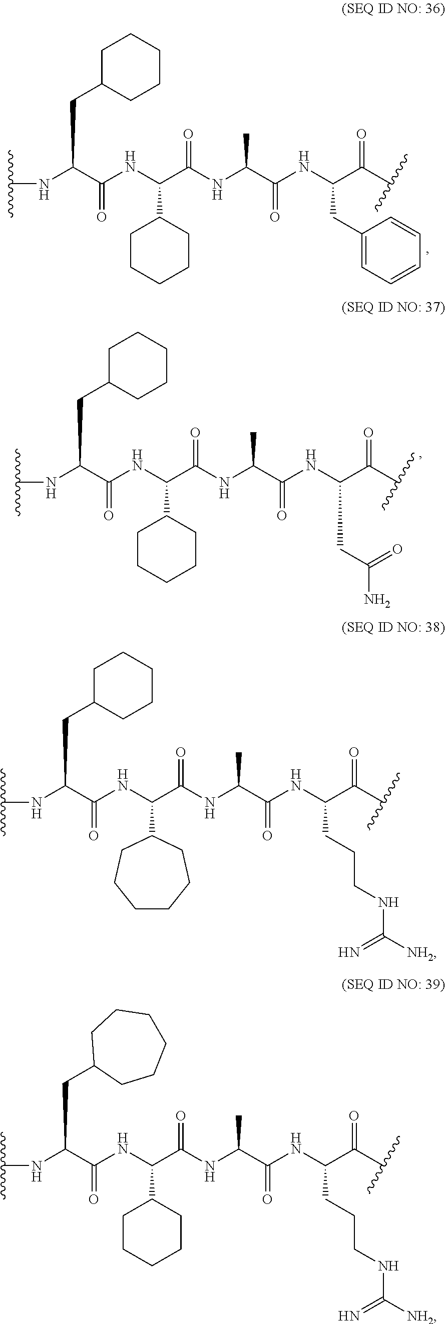 [SMILES: *N[C@@H](CC1CCCCC1)C(=O)N[C@H](C(=O)N[C@@H](C)C(=O)N[C@@H](Cc1ccccc1)C(*)=O)C1CCCCC1 ; *N[C@@H](CC1CCCCC1)C(=O)N[C@H](C(=O)N[C@@H](C)C(=O)N[C@@H](CC(N)=O)C(*)=O)C1CCCCC1 ; *N[C@@H](CC1CCCCC1)C(=O)N[C@H](C(=O)N[C@@H](C)C(=O)N[C@@H](CCCNC(=N)N)C(*)=O)C1CCCCCC1 ; *N[C@@H](CC1CCCCCC1)C(=O)N[C@H](C(=O)N[C@@H](C)C(=O)N[C@@H](CCCNC(=N)N)C(*)=O)C1CCCCC1]

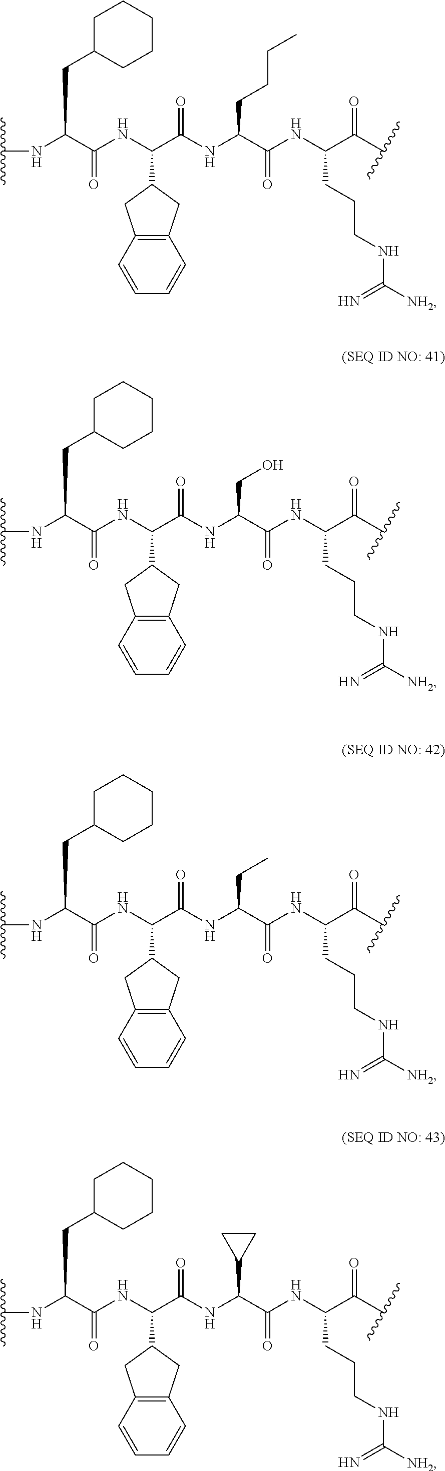 [SMILES: *N[C@@H](CC1CCCCC1)C(=O)N[C@H](C(=O)N[C@@H](CCCC)C(=O)N[C@@H](CCCNC(=N)N)C(*)=O)C1Cc2ccccc2C1 ; *N[C@@H](CC1CCCCC1)C(=O)N[C@H](C(=O)N[C@@H](CO)C(=O)N[C@@H](CCCNC(=N)N)C(*)=O)C1Cc2ccccc2C1 ; *N[C@@H](CC1CCCCC1)C(=O)N[C@H](C(=O)N[C@@H](CC)C(=O)N[C@@H](CCCNC(=N)N)C(*)=O)C1Cc2ccccc2C1 ; *N[C@@H](CC1CCCCC1)C(=O)N[C@H](C(=O)N[C@H](C(=O)N[C@@H](CCCNC(=N)N)C(*)=O)C1CC1)C1Cc2ccccc2C1]

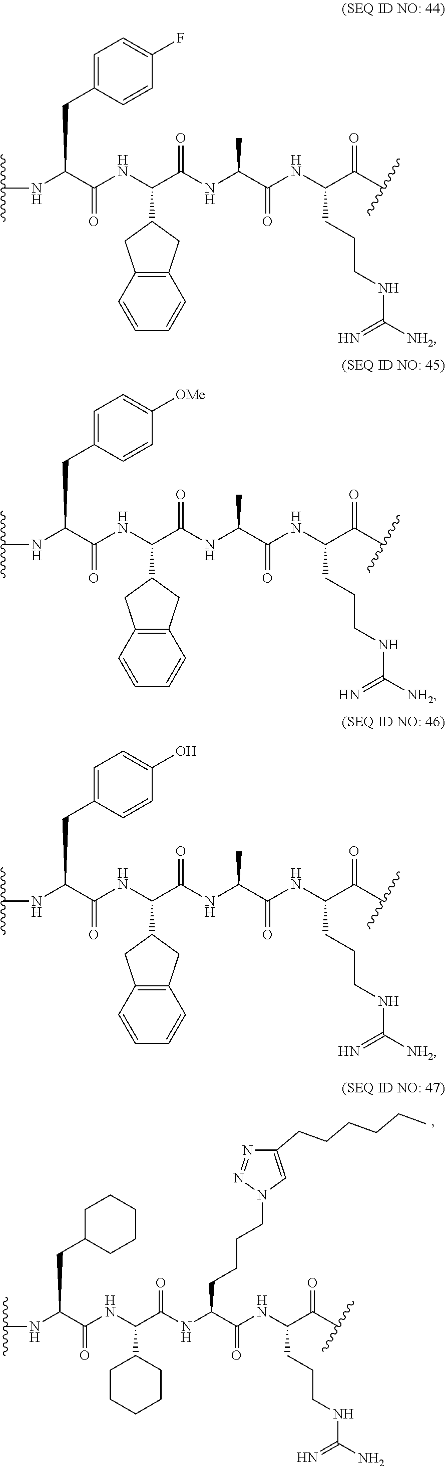 [SMILES: *N[C@@H](Cc1ccc(F)cc1)C(=O)N[C@H](C(=O)N[C@@H](C)C(=O)N[C@@H](CCCNC(=N)N)C(*)=O)C1Cc2ccccc2C1 ; *N[C@@H](Cc1ccc(OC)cc1)C(=O)N[C@H](C(=O)N[C@@H](C)C(=O)N[C@@H](CCCNC(=N)N)C(*)=O)C1Cc2ccccc2C1 ; *N[C@@H](Cc1ccc(O)cc1)C(=O)N[C@H](C(=O)N[C@@H](C)C(=O)N[C@@H](CCCNC(=N)N)C(*)=O)C1Cc2ccccc2C1 ; *N[C@@H](CC1CCCCC1)C(=O)N[C@H](C(=O)N[C@@H](CCCCn1cc(CCCCCC)nn1)C(=O)N[C@@H](CCCNC(=N)N)C(*)=O)C1CCCCC1]

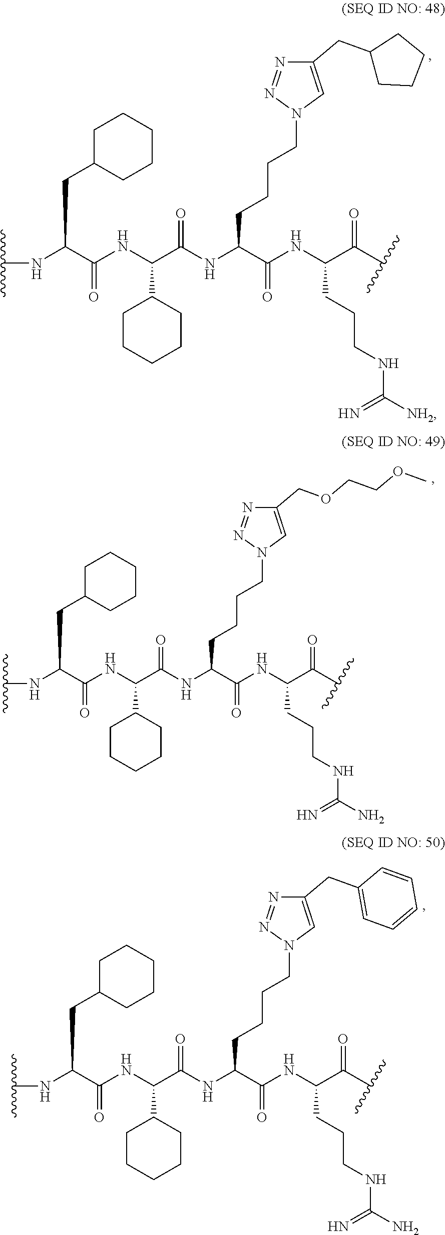 [SMILES: *N[C@@H](CC1CCCCC1)C(=O)N[C@H](C(=O)N[C@@H](CCCCn1cc(CC2CCCC2)nn1)C(=O)N[C@@H](CCCNC(=N)N)C(*)=O)C1CCCCC1 ; *N[C@@H](CC1CCCCC1)C(=O)N[C@H](C(=O)N[C@@H](CCCCn1cc(COCCOC)nn1)C(=O)N[C@@H](CCCNC(=N)N)C(*)=O)C1CCCCC1 ; *N[C@@H](CC1CCCCC1)C(=O)N[C@H](C(=O)N[C@@H](CCCCn1cc(Cc2ccccc2)nn1)C(=O)N[C@@H](CCCNC(=N)N)C(*)=O)C1CCCCC1]

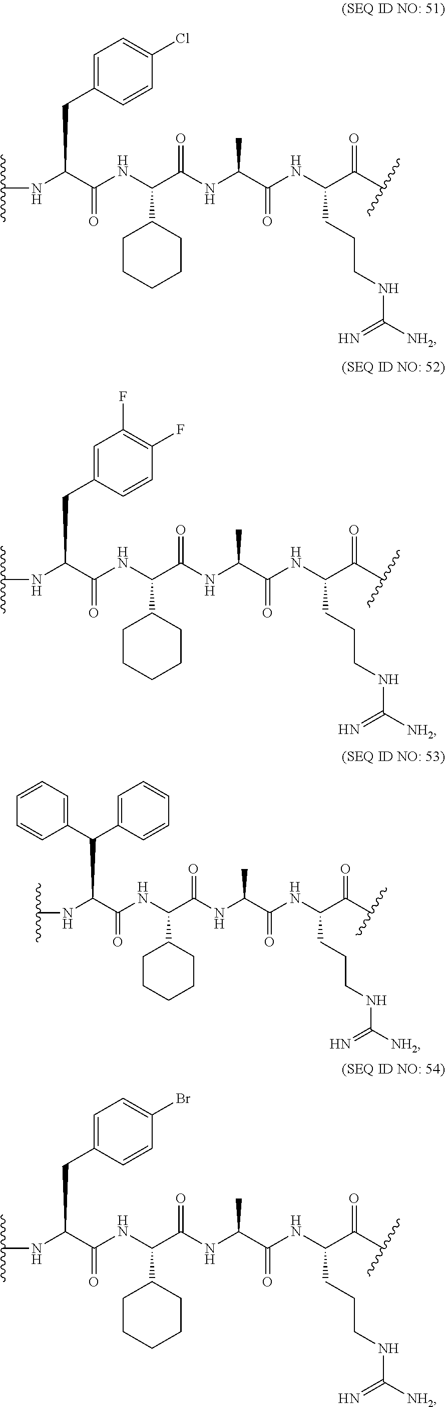 [SMILES: *N[C@@H](Cc1ccc(Cl)cc1)C(=O)N[C@H](C(=O)N[C@@H](C)C(=O)N[C@@H](CCCNC(=N)N)C(*)=O)C1CCCCC1 ; *N[C@@H](Cc1ccc(F)c(F)c1)C(=O)N[C@H](C(=O)N[C@@H](C)C(=O)N[C@@H](CCCNC(=N)N)C(*)=O)C1CCCCC1 ; *N[C@H](C(=O)N[C@H](C(=O)N[C@@H](C)C(=O)N[C@@H](CCCNC(=N)N)C(*)=O)C1CCCCC1)C(c1ccccc1)c1ccccc1 ; *N[C@@H](Cc1ccc(Br)cc1)C(=O)N[C@H](C(=O)N[C@@H](C)C(=O)N[C@@H](CCCNC(=N)N)C(*)=O)C1CCCCC1]

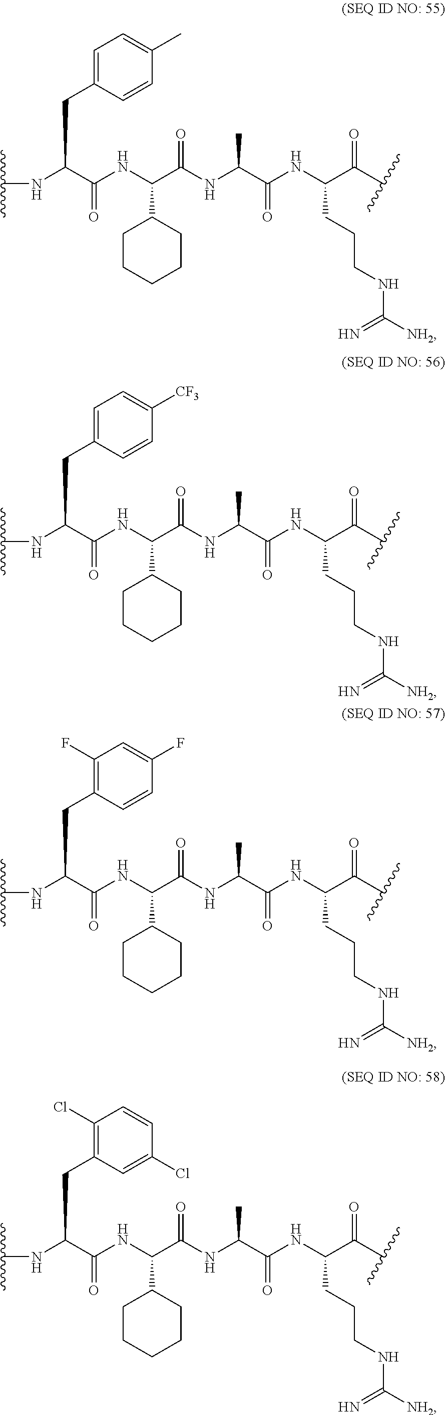 [SMILES: *N[C@@H](Cc1ccc(C)cc1)C(=O)N[C@H](C(=O)N[C@@H](C)C(=O)N[C@@H](CCCNC(=N)N)C(*)=O)C1CCCCC1 ; *N[C@@H](Cc1ccc(C(F)(F)F)cc1)C(=O)N[C@H](C(=O)N[C@@H](C)C(=O)N[C@@H](CCCNC(=N)N)C(*)=O)C1CCCCC1 ; *N[C@@H](Cc1ccc(F)cc1F)C(=O)N[C@H](C(=O)N[C@@H](C)C(=O)N[C@@H](CCCNC(=N)N)C(*)=O)C1CCCCC1 ; *N[C@@H](Cc1cc(Cl)ccc1Cl)C(=O)N[C@H](C(=O)N[C@@H](C)C(=O)N[C@@H](CCCNC(=N)N)C(*)=O)C1CCCCC1]

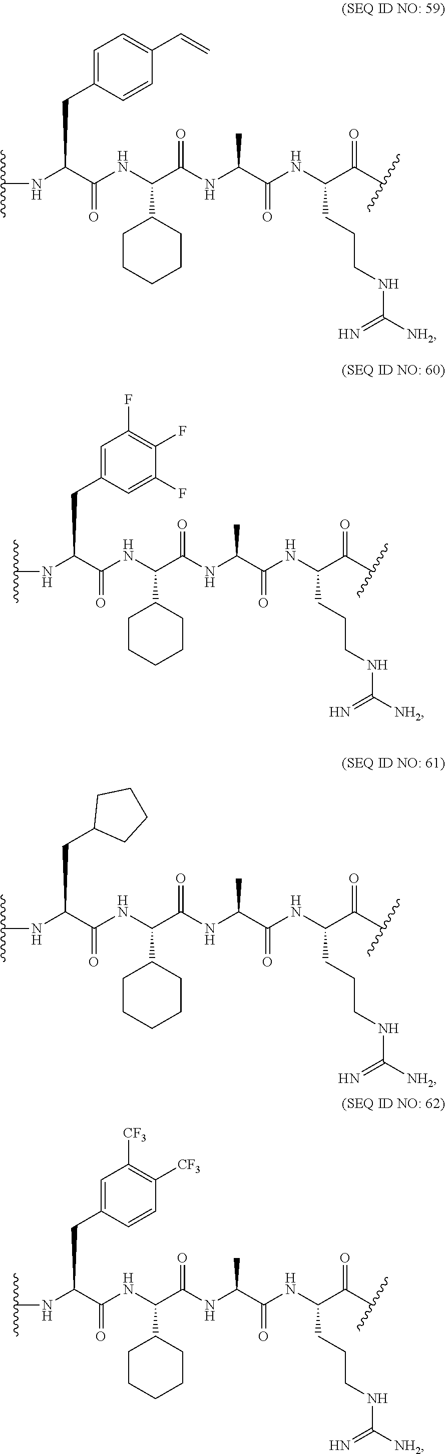 [SMILES: *N[C@@H](Cc1ccc(C=C)cc1)C(=O)N[C@H](C(=O)N[C@@H](C)C(=O)N[C@@H](CCCNC(=N)N)C(*)=O)C1CCCCC1 ; *N[C@@H](Cc1cc(F)c(F)c(F)c1)C(=O)N[C@H](C(=O)N[C@@H](C)C(=O)N[C@@H](CCCNC(=N)N)C(*)=O)C1CCCCC1 ; *N[C@@H](CC1CCCC1)C(=O)N[C@H](C(=O)N[C@@H](C)C(=O)N[C@@H](CCCNC(=N)N)C(*)=O)C1CCCCC1 ; *N[C@@H](Cc1ccc(C(F)(F)F)c(C(F)(F)F)c1)C(=O)N[C@H](C(=O)N[C@@H](C)C(=O)N[C@@H](CCCNC(=N)N)C(*)=O)C1CCCCC1]

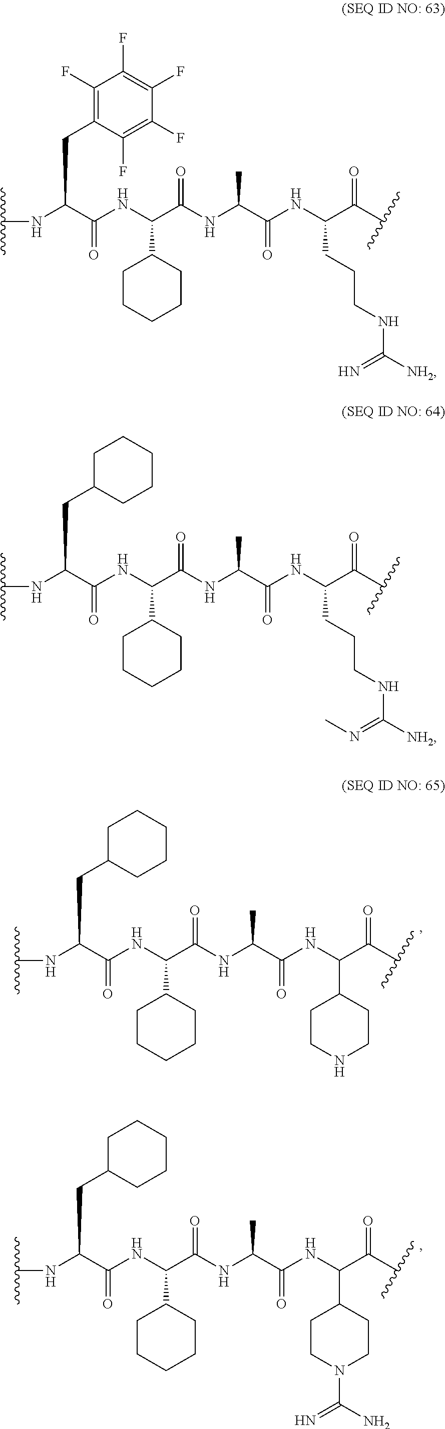 [SMILES: *N[C@@H](Cc1c(F)c(F)c(F)c(F)c1F)C(=O)N[C@H](C(=O)N[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N[C@@H](CCCN/C(N)=N\C)C(*)=O)C1CCCCC1 ; *N[C@@H](CC1CCCCC1)C(=O)N[C@H](C(=O)N[C@@H](C)C(=O)NC(C(*)=O)C1CCNCC1)C1CCCCC1 ; *N[C@@H](CC1CCCCC1)C(=O)N[C@H](C(=O)N[C@@H](C)C(=O)NC(C(*)=O)C1CCN(C(=N)N)CC1)C1CCCCC1]

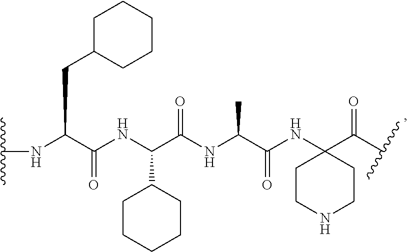 [SMILES: *N[C@@H](CC1CCCCC1)C(=O)N[C@H](C(=O)N[C@@H](C)C(=O)NC1(C(*)=O)CCNCC1)C1CCCCC1]

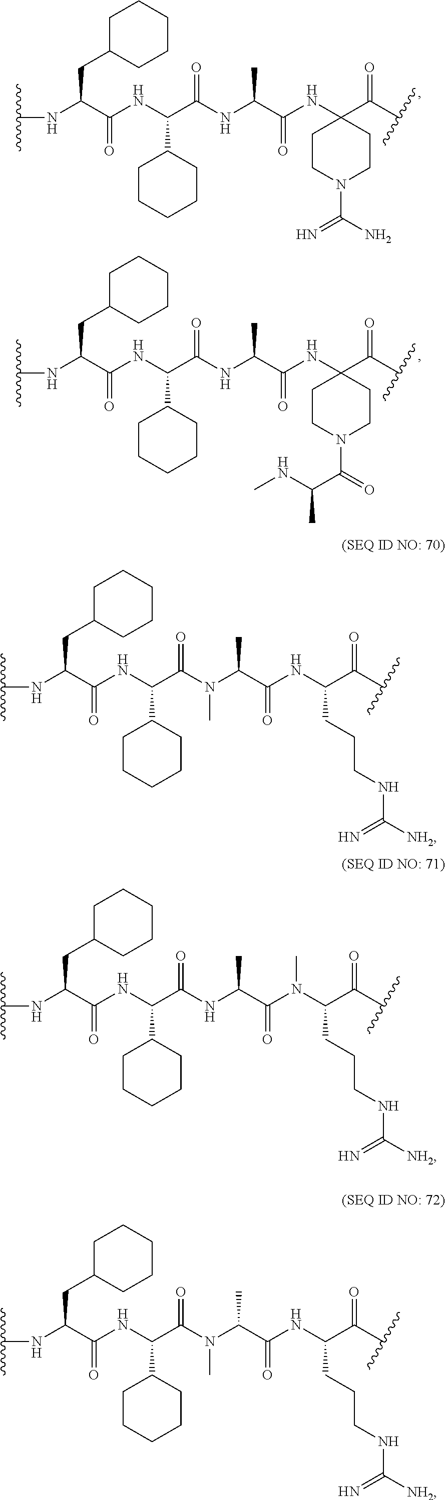 [SMILES: *N[C@@H](CC1CCCCC1)C(=O)N[C@H](C(=O)N[C@@H](C)C(=O)NC1(C(*)=O)CCN(C(=N)N)CC1)C1CCCCC1 ; *N[C@@H](CC1CCCCC1)C(=O)N[C@H](C(=O)N[C@@H](C)C(=O)NC1(C(*)=O)CCN(C(=O)[C@@H](C)NC)CC1)C1CCCCC1 ; *N[C@@H](CC1CCCCC1)C(=O)N[C@H](C(=O)N(C)[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N(C)[C@@H](CCCNC(=N)N)C(*)=O)C1CCCCC1 ; *N[C@@H](CC1CCCCC1)C(=O)N[C@H](C(=O)N(C)[C@H](C)C(=O)N[C@@H](CCCNC(=N)N)C(*)=O)C1CCCCC1]

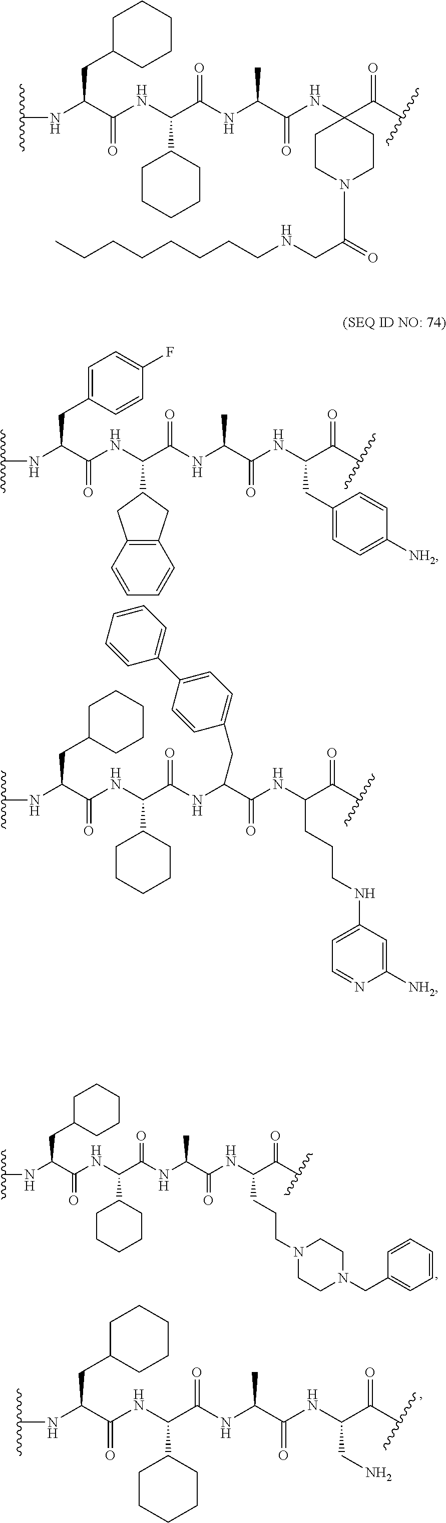

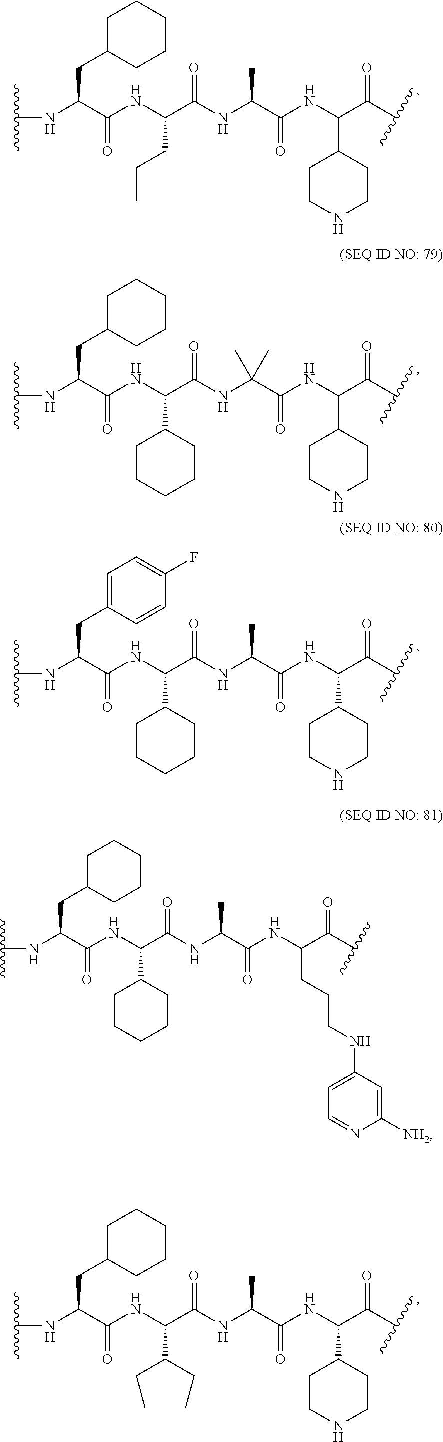 [SMILES: *N[C@@H](CC1CCCCC1)C(=O)N[C@@H](CCC)C(=O)N[C@@H](C)C(=O)NC(C(*)=O)C1CCNCC1 ; *N[C@@H](CC1CCCCC1)C(=O)N[C@H](C(=O)NC(C)(C)C(=O)NC(C(*)=O)C1CCNCC1)C1CCCCC1 ; *N[C@@H](Cc1ccc(F)cc1)C(=O)N[C@H](C(=O)N[C@@H](C)C(=O)N[C@H](C(*)=O)C1CCNCC1)C1CCCCC1 ; *N[C@@H](CC1CCCCC1)C(=O)N[C@H](C(=O)N[C@@H](C)C(=O)NC(CCCNc1ccnc(N)c1)C(*)=O)C1CCCCC1 ; *N[C@@H](CC1CCCCC1)C(=O)N[C@H](C(=O)N[C@@H](C)C(=O)N[C@H](C(*)=O)C1CCNCC1)C(CC)CC]

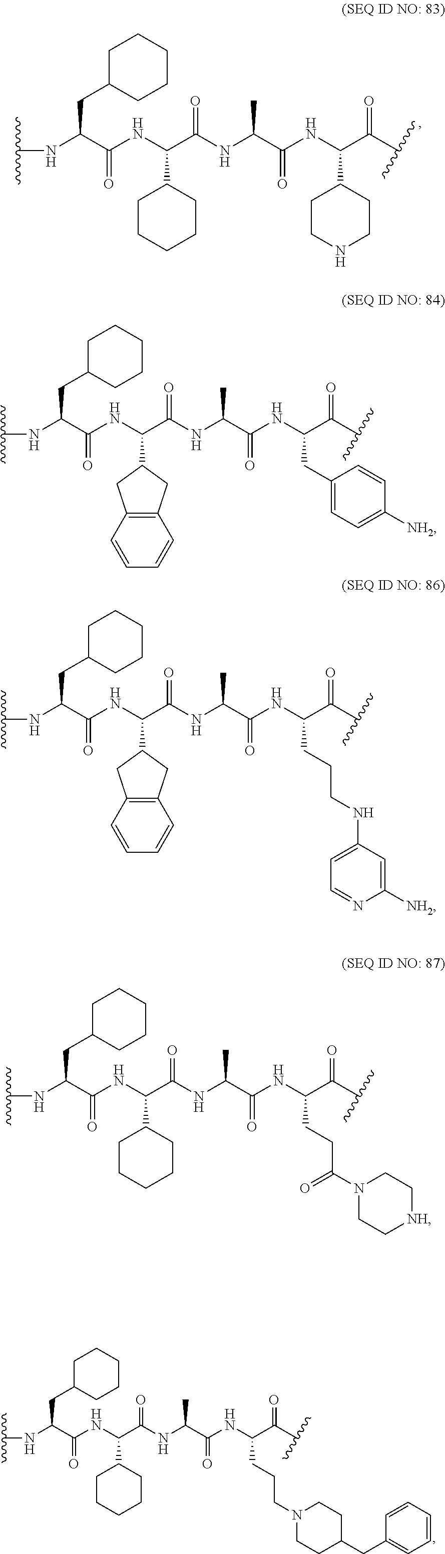 [SMILES: *N[C@@H](CC1CCCCC1)C(=O)N[C@H](C(=O)N[C@@H](C)C(=O)N[C@H](C(*)=O)C1CCNCC1)C1CCCCC1 ; *N[C@@H](CC1CCCCC1)C(=O)N[C@H](C(=O)N[C@@H](C)C(=O)N[C@@H](Cc1ccc(N)cc1)C(*)=O)C1Cc2ccccc2C1 ; *N[C@@H](CC1CCCCC1)C(=O)N[C@H](C(=O)N[C@@H](C)C(=O)N[C@@H](CCCNc1ccnc(N)c1)C(*)=O)C1Cc2ccccc2C1 ; *N[C@@H](CC1CCCCC1)C(=O)N[C@H](C(=O)N[C@@H](C)C(=O)N[C@@H](CCC(=O)N1CCNCC1)C(*)=O)C1CCCCC1 ; *N[C@@H](CC1CCCCC1)C(=O)N[C@H](C(=O)N[C@@H](C)C(=O)N[C@@H](CCCN1CCC(Cc2ccccc2)CC1)C(*)=O)C1CCCCC1]

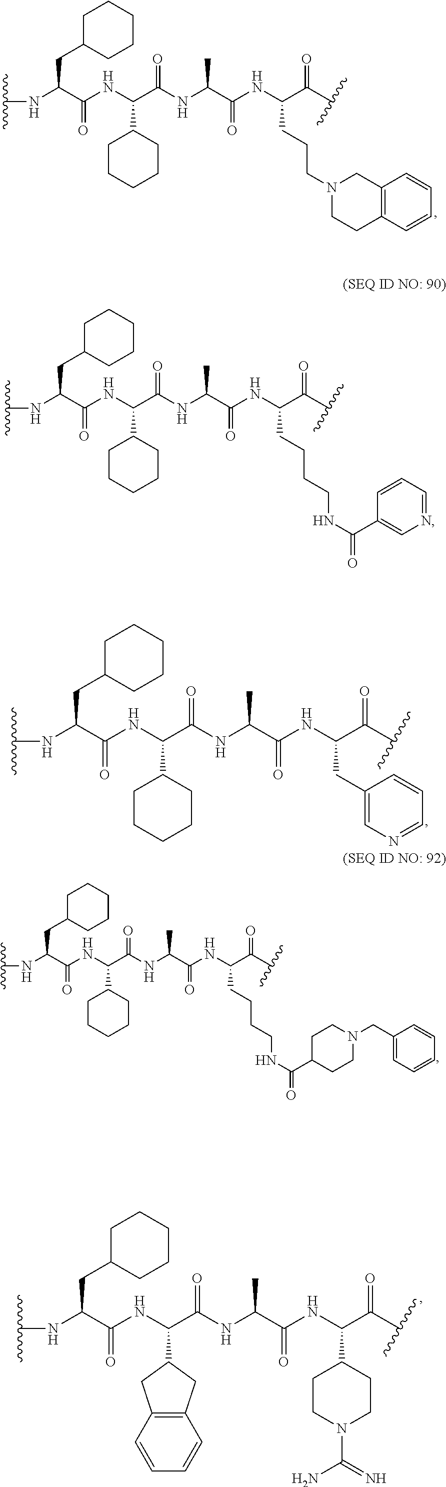 [SMILES: *N[C@@H](CC1CCCCC1)C(=O)N[C@H](C(=O)N[C@@H](C)C(=O)N[C@@H](CCCN1CCc2ccccc2C1)C(*)=O)C1CCCCC1 ; *N[C@@H](CC1CCCCC1)C(=O)N[C@H](C(=O)N[C@@H](C)C(=O)N[C@@H](CCCCNC(=O)c1cccnc1)C(*)=O)C1CCCCC1 ; *N[C@@H](CC1CCCCC1)C(=O)N[C@H](C(=O)N[C@@H](C)C(=O)N[C@@H](Cc1cccnc1)C(*)=O)C1CCCCC1 ; *N[C@@H](CC1CCCCC1)C(=O)N[C@H](C(=O)N[C@@H](C)C(=O)N[C@@H](CCCCNC(=O)C1CCN(Cc2ccccc2)CC1)C(*)=O)C1CCCCC1 ; *N[C@@H](CC1CCCCC1)C(=O)N[C@H](C(=O)N[C@@H](C)C(=O)N[C@H](C(*)=O)C1CCN(C(=N)N)CC1)C1Cc2ccccc2C1]

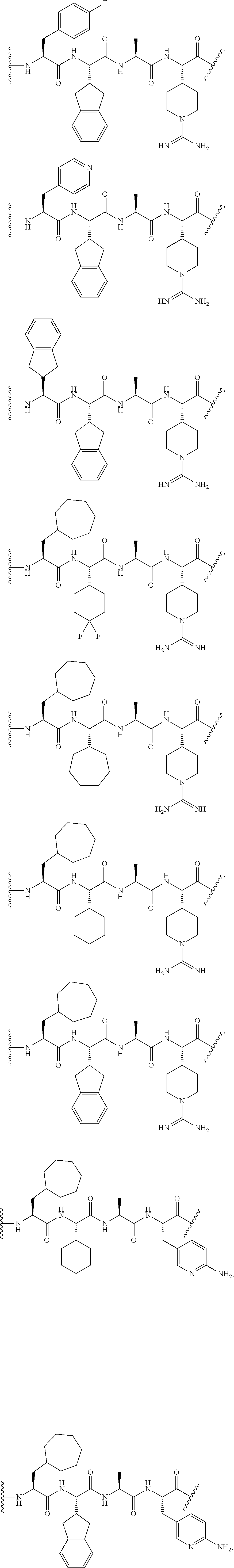

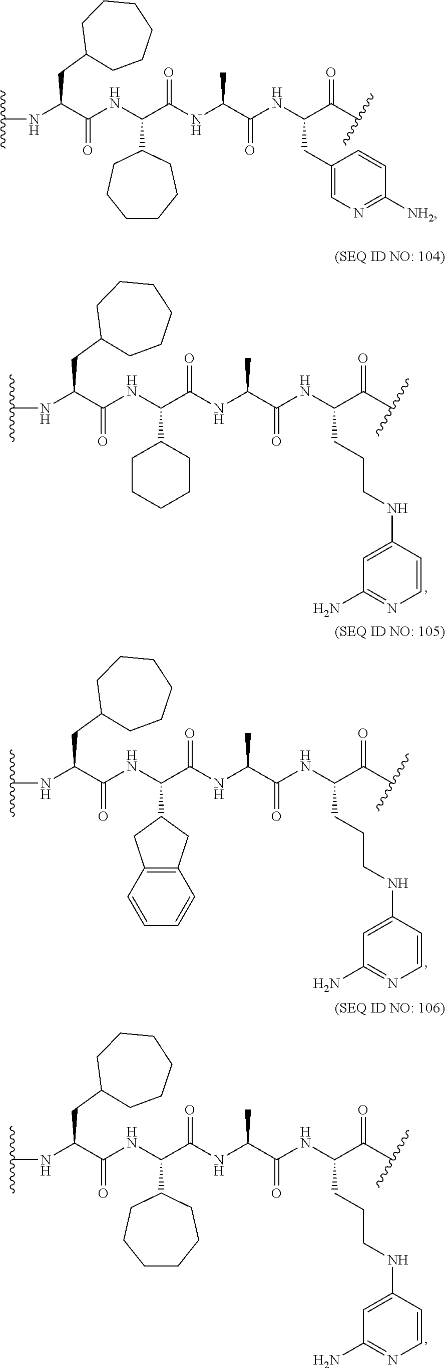 [SMILES: *N[C@@H](CC1CCCCCC1)C(=O)N[C@H](C(=O)N[C@@H](C)C(=O)N[C@@H](Cc1ccc(N)nc1)C(*)=O)C1CCCCCC1 ; *N[C@@H](CC1CCCCCC1)C(=O)N[C@H](C(=O)N[C@@H](C)C(=O)N[C@@H](CCCNc1ccnc(N)c1)C(*)=O)C1CCCCC1 ; *N[C@@H](CC1CCCCCC1)C(=O)N[C@H](C(=O)N[C@@H](C)C(=O)N[C@@H](CCCNc1ccnc(N)c1)C(*)=O)C1Cc2ccccc2C1 ; *N[C@@H](CC1CCCCCC1)C(=O)N[C@H](C(=O)N[C@@H](C)C(=O)N[C@@H](CCCNc1ccnc(N)c1)C(*)=O)C1CCCCCC1]

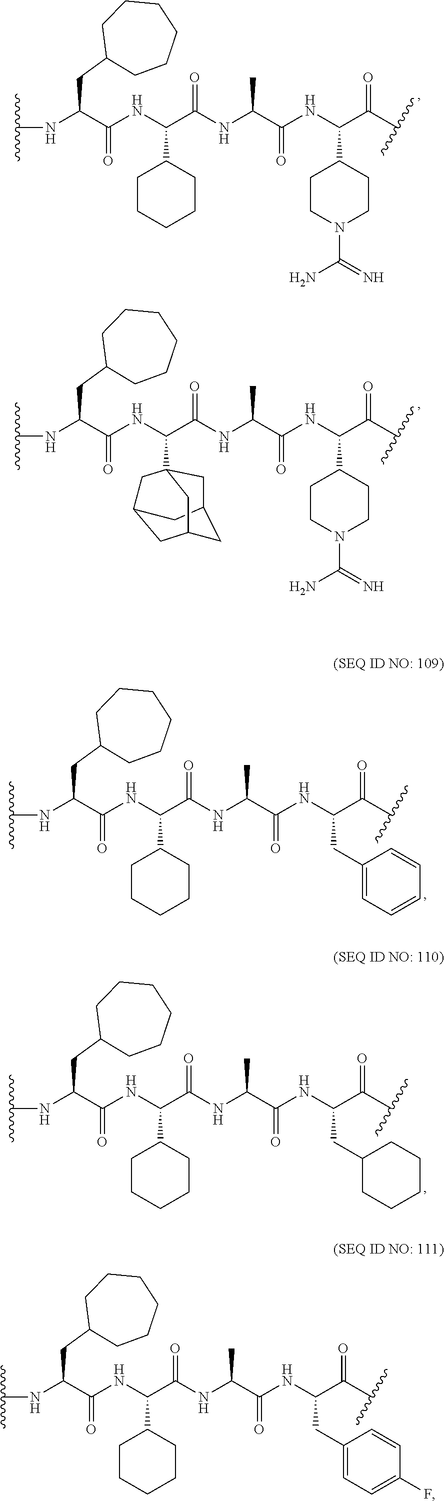 [SMILES: *N[C@@H](CC1CCCCCC1)C(=O)N[C@H](C(=O)N[C@@H](C)C(=O)N[C@H](C(*)=O)C1CCN(C(=N)N)CC1)C1CCCCC1 ; *N[C@@H](CC1CCCCCC1)C(=O)N[C@H](C(=O)N[C@@H](C)C(=O)N[C@H](C(*)=O)C1CCN(C(=N)N)CC1)C12CC3CC(CC(C3)C1)C2 ; *N[C@@H](CC1CCCCCC1)C(=O)N[C@H](C(=O)N[C@@H](C)C(=O)N[C@@H](Cc1ccccc1)C(*)=O)C1CCCCC1 ; *N[C@@H](CC1CCCCCC1)C(=O)N[C@H](C(=O)N[C@@H](C)C(=O)N[C@@H](CC1CCCCC1)C(*)=O)C1CCCCC1 ; *N[C@@H](CC1CCCCCC1)C(=O)N[C@H](C(=O)N[C@@H](C)C(=O)N[C@@H](Cc1ccc(F)cc1)C(*)=O)C1CCCCC1]

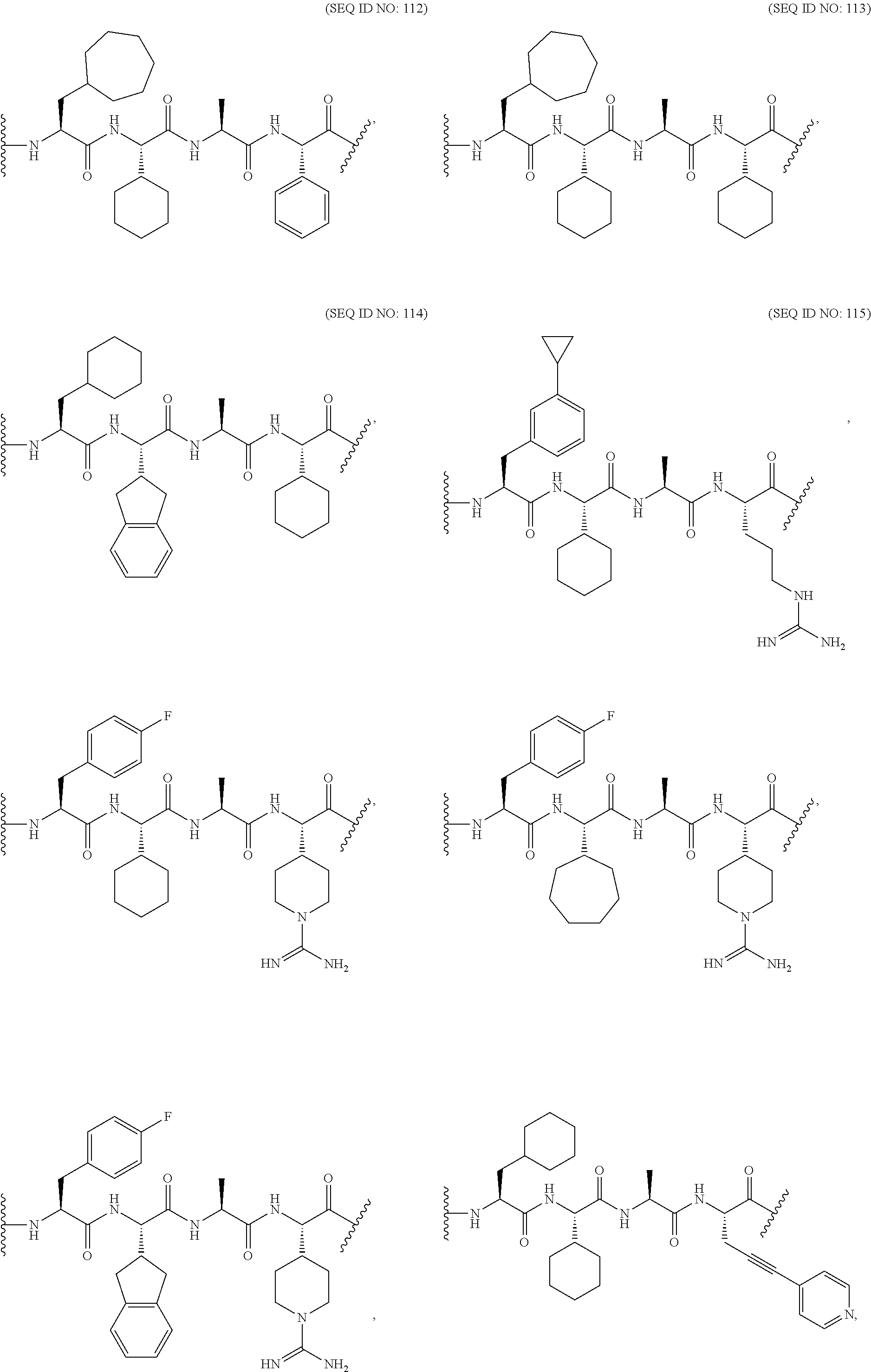

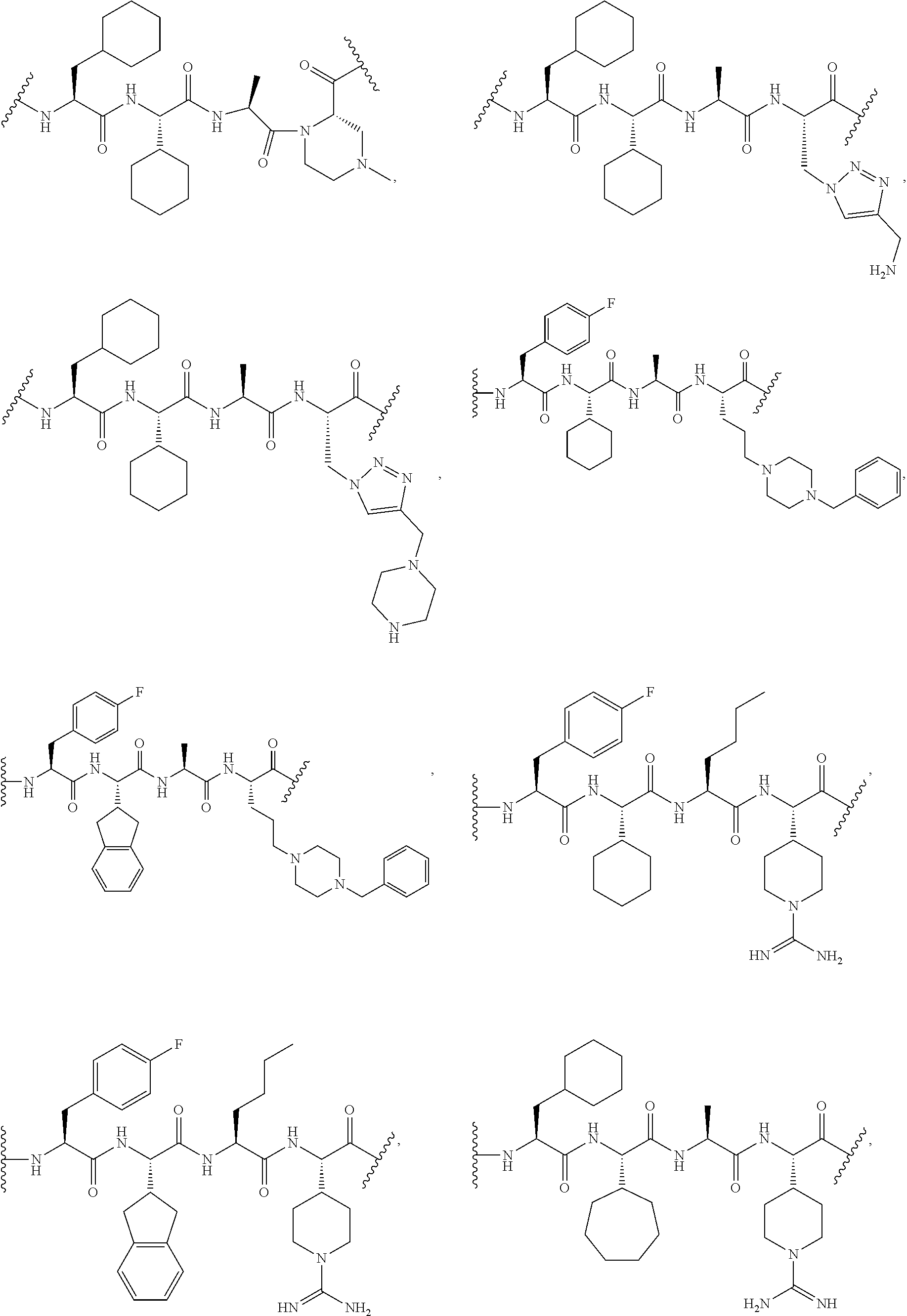

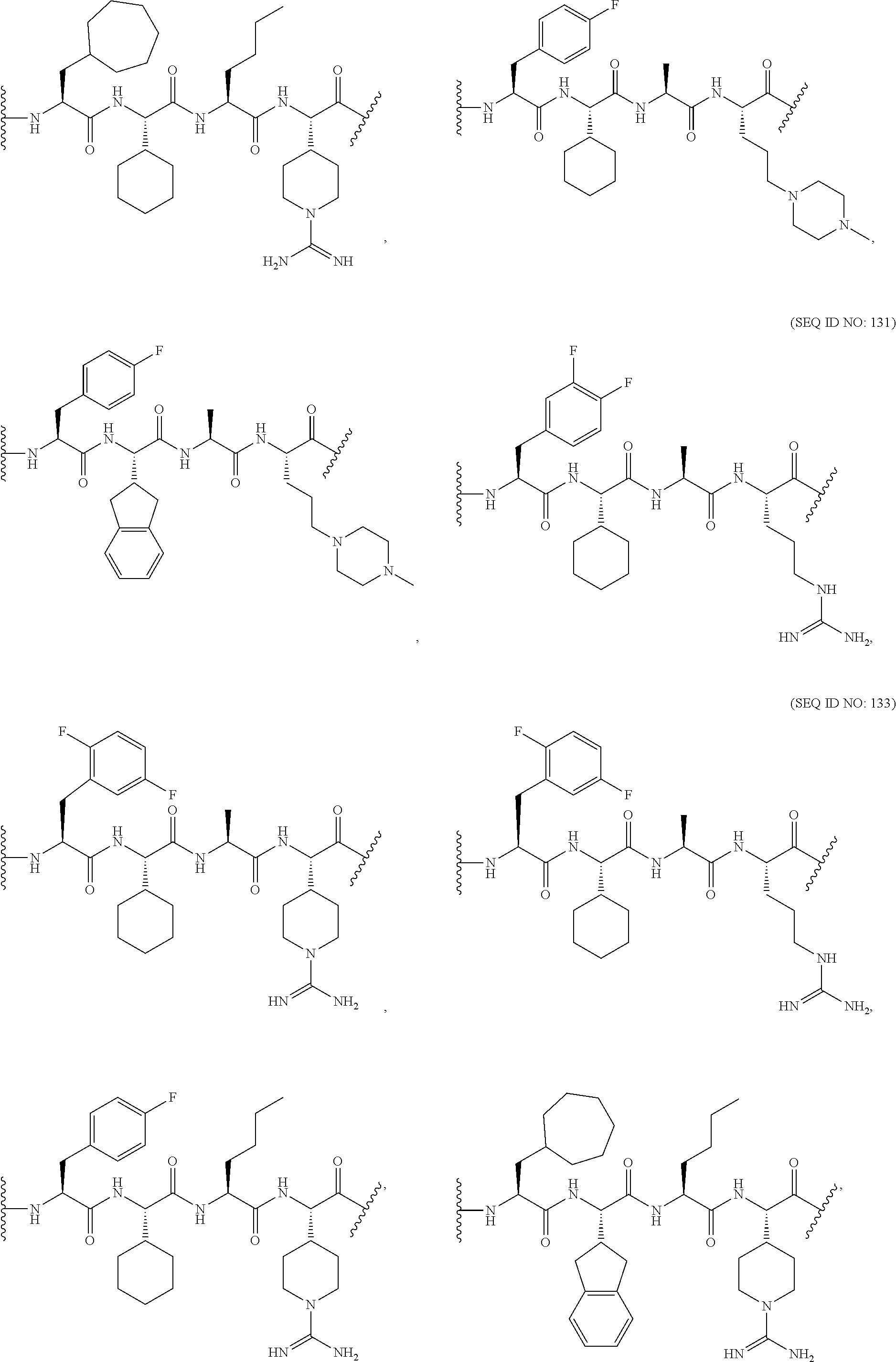

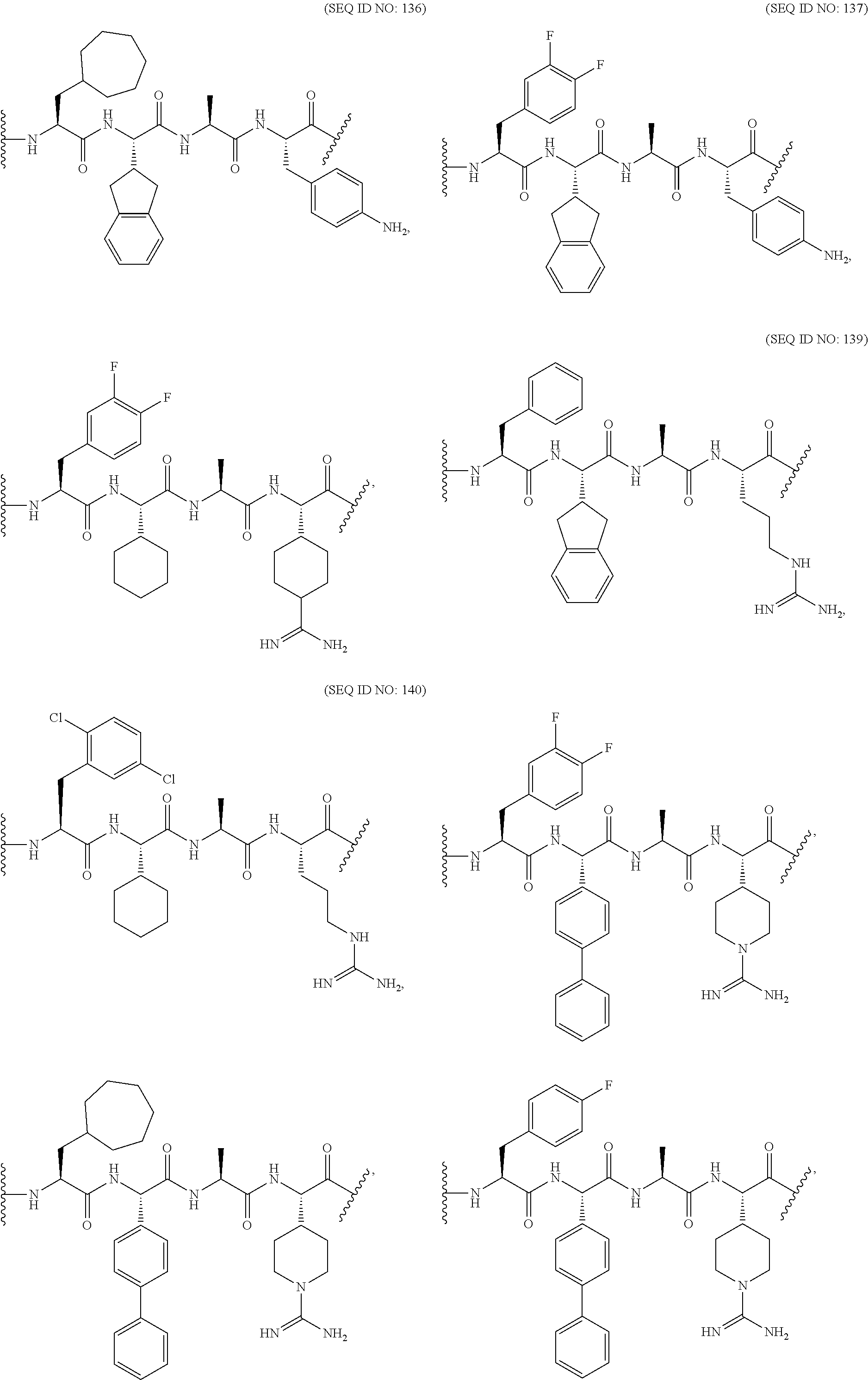

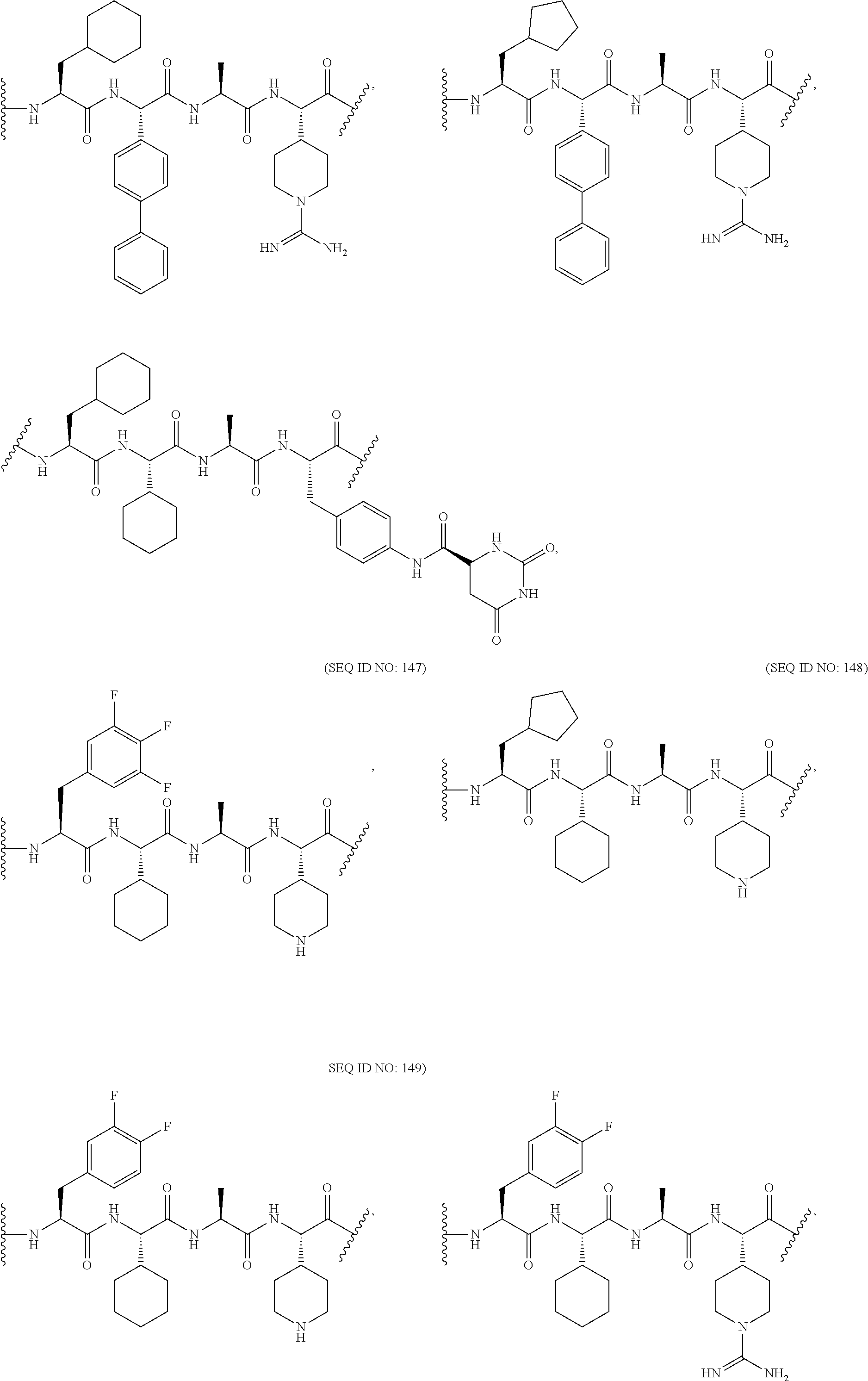

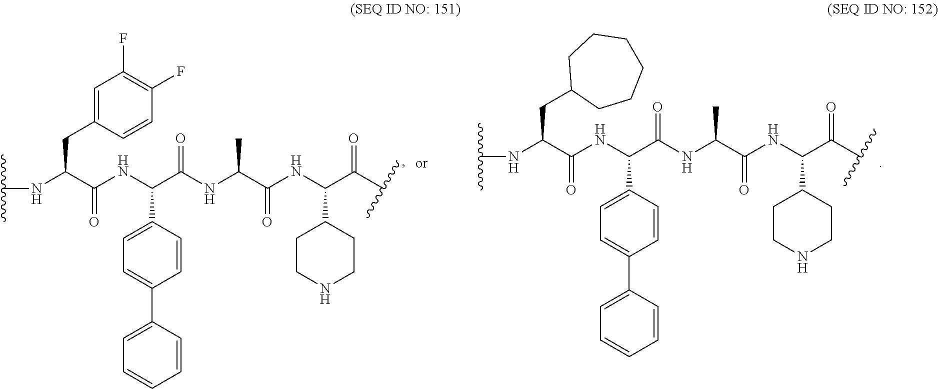 [SMILES: *N[C@@H](Cc1ccc(F)c(F)c1)C(=O)N[C@H](C(=O)N[C@@H](C)C(=O)N[C@H](C(*)=O)C1CCNCC1)c1ccc(-c2ccccc2)cc1 ; *N[C@@H](CC1CCCCCC1)C(=O)N[C@H](C(=O)N[C@@H](C)C(=O)N[C@H](C(*)=O)C1CCNCC1)c1ccc(-c2ccccc2)cc1]

In some embodiments, —X¹—X²—X³—X⁴— is:

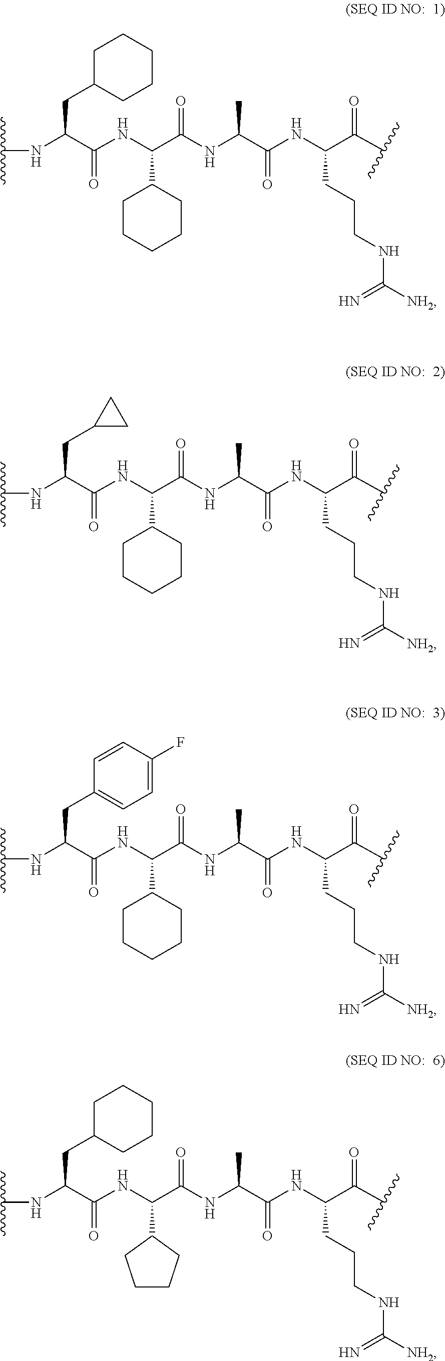 [SMILES: *N[C@@H](CC1CCCCC1)C(=O)N[C@H](C(=O)N[C@@H](C)C(=O)N[C@@H](CCCNC(=N)N)C(*)=O)C1CCCCC1 ; *N[C@@H](CC1CC1)C(=O)N[C@H](C(=O)N[C@@H](C)C(=O)N[C@@H](CCCNC(=N)N)C(*)=O)C1CCCCC1 ; *N[C@@H](Cc1ccc(F)cc1)C(=O)N[C@H](C(=O)N[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N[C@@H](CCCNC(=N)N)C(*)=O)C1CCCC1]

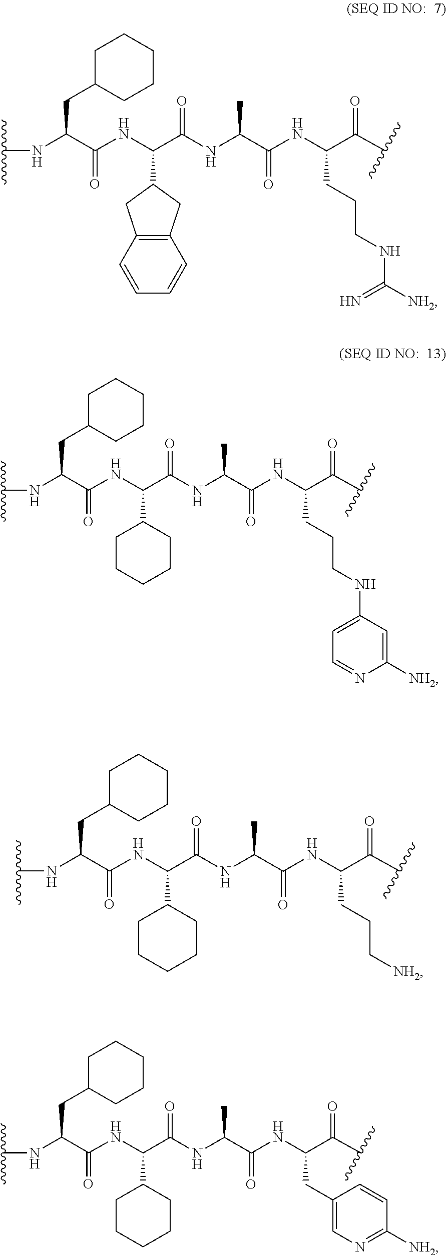 [SMILES: *N[C@@H](CC1CCCCC1)C(=O)N[C@H](C(=O)N[C@@H](C)C(=O)N[C@@H](CCCNC(=N)N)C(*)=O)C1Cc2ccccc2C1 ; *N[C@@H](CC1CCCCC1)C(=O)N[C@H](C(=O)N[C@@H](C)C(=O)N[C@@H](CCCNc1ccnc(N)c1)C(*)=O)C1CCCCC1 ; *N[C@@H](CC1CCCCC1)C(=O)N[C@H](C(=O)N[C@@H](C)C(=O)N[C@@H](CCCN)C(*)=O)C1CCCCC1 ; *N[C@@H](CC1CCCCC1)C(=O)N[C@H](C(=O)N[C@@H](C)C(=O)N[C@@H](Cc1ccc(N)nc1)C(*)=O)C1CCCCC1]

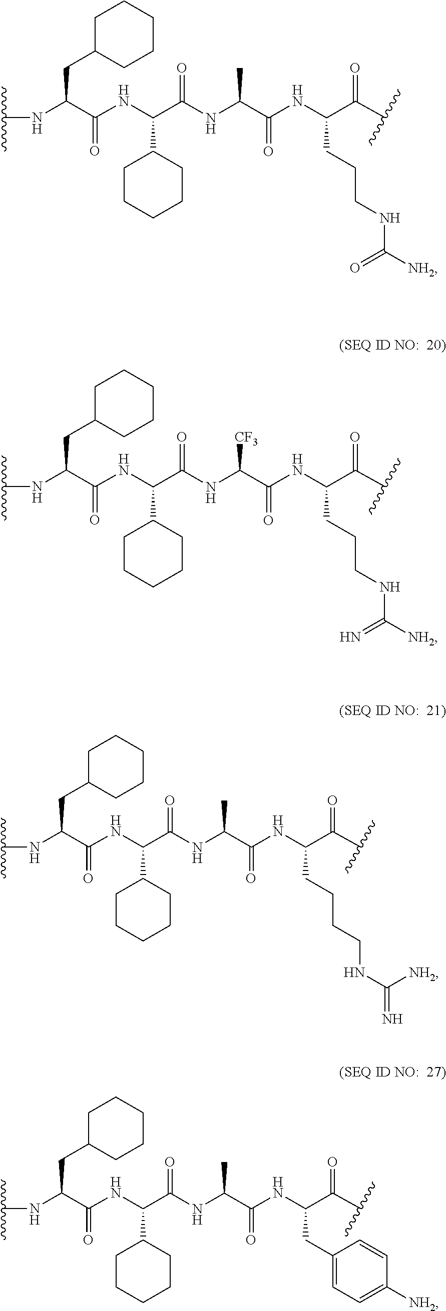 [SMILES: *N[C@@H](CC1CCCCC1)C(=O)N[C@H](C(=O)N[C@@H](C)C(=O)N[C@@H](CCCNC(N)=O)C(*)=O)C1CCCCC1 ; *N[C@@H](CC1CCCCC1)C(=O)N[C@H](C(=O)N[C@H](C(=O)N[C@@H](CCCNC(=N)N)C(*)=O)C(F)(F)F)C1CCCCC1 ; *N[C@@H](CC1CCCCC1)C(=O)N[C@H](C(=O)N[C@@H](C)C(=O)N[C@@H](CCCCNC(=N)N)C(*)=O)C1CCCCC1 ; *N[C@@H](CC1CCCCC1)C(=O)N[C@H](C(=O)N[C@@H](C)C(=O)N[C@@H](Cc1ccc(N)cc1)C(*)=O)C1CCCCC1]

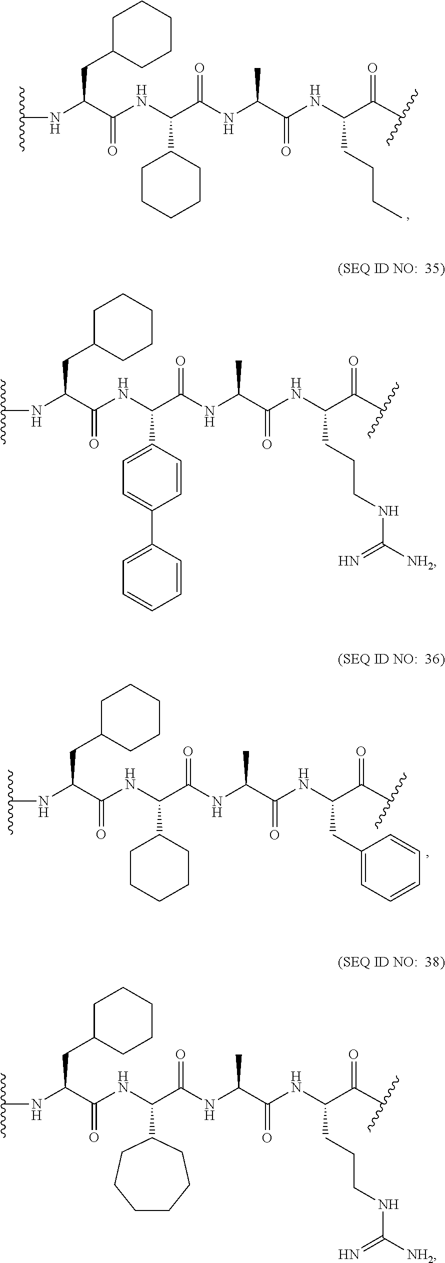 [SMILES: *N[C@@H](CC1CCCCC1)C(=O)N[C@H](C(=O)N[C@@H](C)C(=O)N[C@@H](CCCC)C(*)=O)C1CCCCC1 ; *N[C@@H](CC1CCCCC1)C(=O)N[C@H](C(=O)N[C@@H](C)C(=O)N[C@@H](CCCNC(=N)N)C(*)=O)c1ccc(-c2ccccc2)cc1 ; *N[C@@H](CC1CCCCC1)C(=O)N[C@H](C(=O)N[C@@H](C)C(=O)N[C@@H](Cc1ccccc1)C(*)=O)C1CCCCC1 ; *N[C@@H](CC1CCCCC1)C(=O)N[C@H](C(=O)N[C@@H](C)C(=O)N[C@@H](CCCNC(=N)N)C(*)=O)C1CCCCCC1]

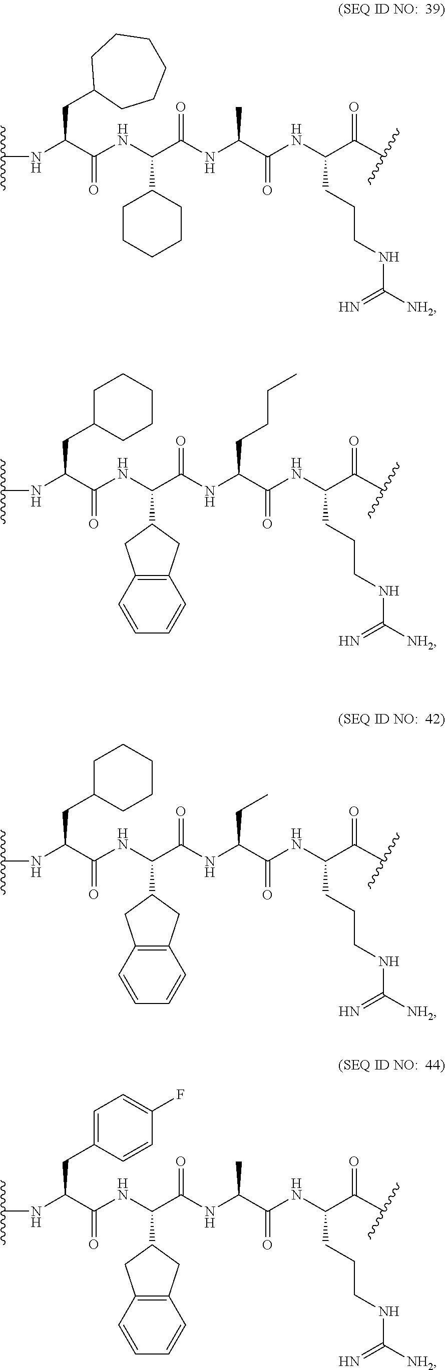 [SMILES: *N[C@@H](CC1CCCCCC1)C(=O)N[C@H](C(=O)N[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](CCCC)C(=O)N[C@@H](CCCNC(=N)N)C(*)=O)C1Cc2ccccc2C1 ; *N[C@@H](CC1CCCCC1)C(=O)N[C@H](C(=O)N[C@@H](CC)C(=O)N[C@@H](CCCNC(=N)N)C(*)=O)C1Cc2ccccc2C1 ; *N[C@@H](Cc1ccc(F)cc1)C(=O)N[C@H](C(=O)N[C@@H](C)C(=O)N[C@@H](CCCNC(=N)N)C(*)=O)C1Cc2ccccc2C1]

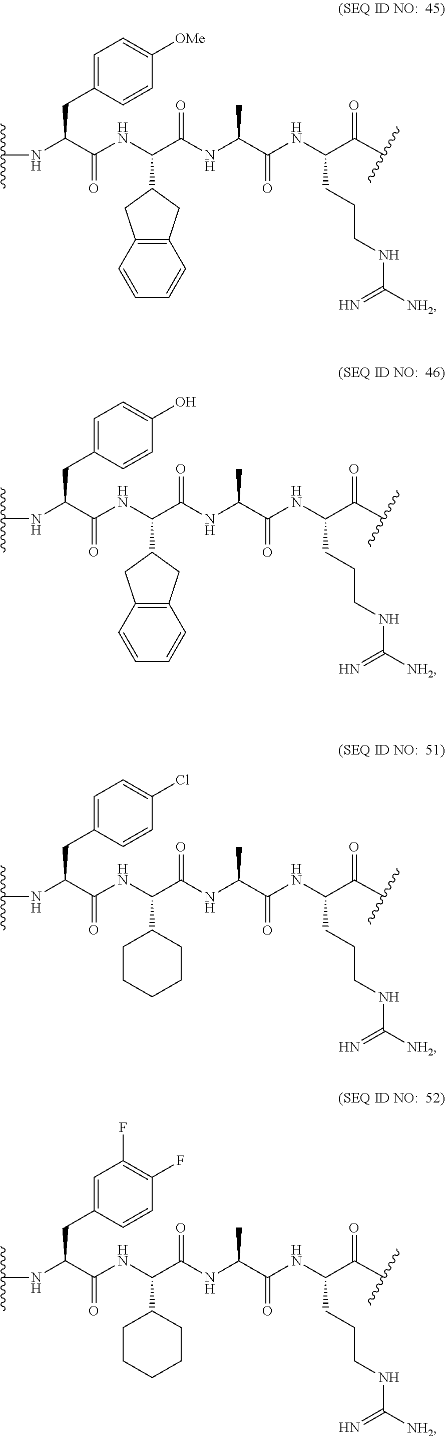 [SMILES: *N[C@@H](Cc1ccc(OC)cc1)C(=O)N[C@H](C(=O)N[C@@H](C)C(=O)N[C@@H](CCCNC(=N)N)C(*)=O)C1Cc2ccccc2C1 ; *N[C@@H](Cc1ccc(O)cc1)C(=O)N[C@H](C(=O)N[C@@H](C)C(=O)N[C@@H](CCCNC(=N)N)C(*)=O)C1Cc2ccccc2C1 ; *N[C@@H](Cc1ccc(Cl)cc1)C(=O)N[C@H](C(=O)N[C@@H](C)C(=O)N[C@@H](CCCNC(=N)N)C(*)=O)C1CCCCC1 ; *N[C@@H](Cc1ccc(F)c(F)c1)C(=O)N[C@H](C(=O)N[C@@H](C)C(=O)N[C@@H](CCCNC(=N)N)C(*)=O)C1CCCCC1]

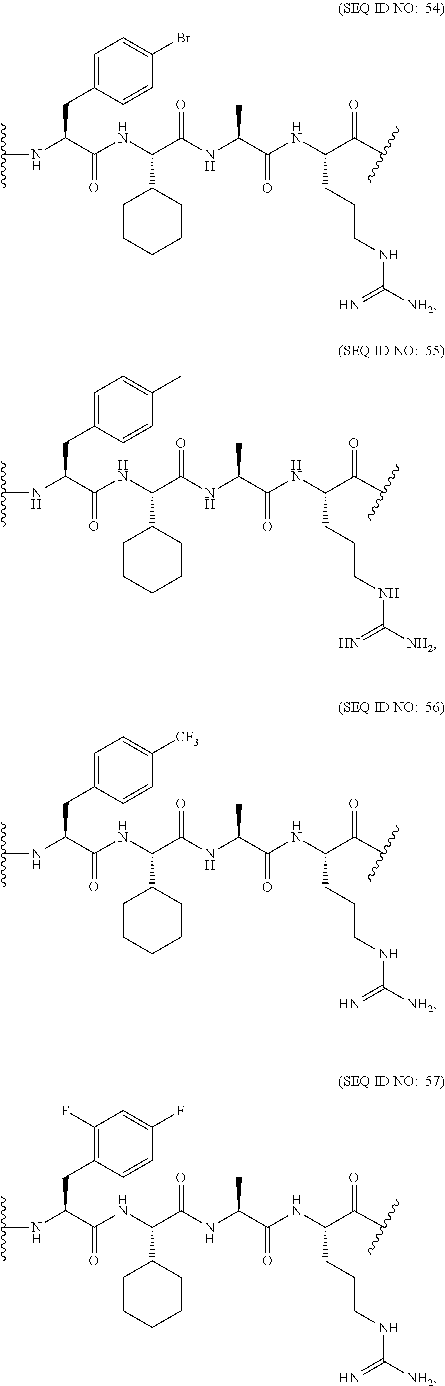 [SMILES: *N[C@@H](Cc1ccc(Br)cc1)C(=O)N[C@H](C(=O)N[C@@H](C)C(=O)N[C@@H](CCCNC(=N)N)C(*)=O)C1CCCCC1 ; *N[C@@H](Cc1ccc(C)cc1)C(=O)N[C@H](C(=O)N[C@@H](C)C(=O)N[C@@H](CCCNC(=N)N)C(*)=O)C1CCCCC1 ; *N[C@@H](Cc1ccc(C(F)(F)F)cc1)C(=O)N[C@H](C(=O)N[C@@H](C)C(=O)N[C@@H](CCCNC(=N)N)C(*)=O)C1CCCCC1 ; *N[C@@H](Cc1ccc(F)cc1F)C(=O)N[C@H](C(=O)N[C@@H](C)C(=O)N[C@@H](CCCNC(=N)N)C(*)=O)C1CCCCC1]

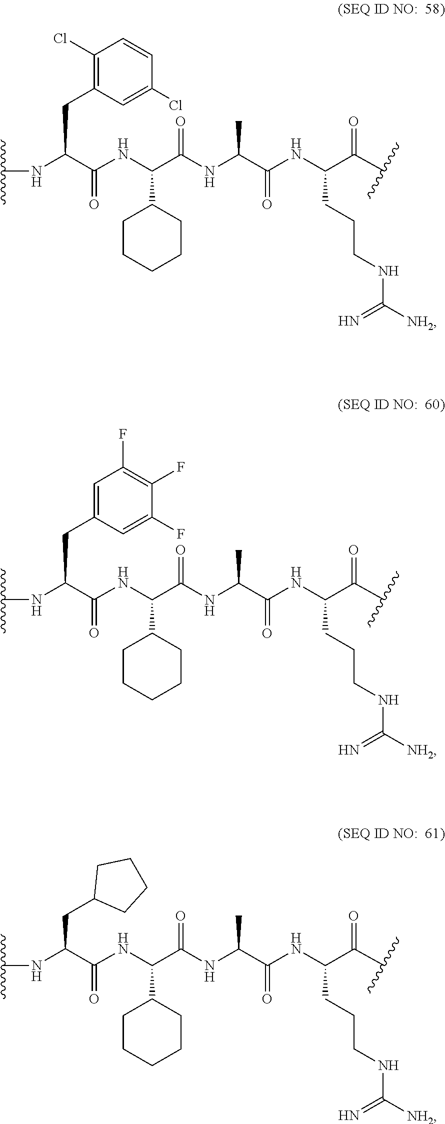 [SMILES: *N[C@@H](Cc1cc(Cl)ccc1Cl)C(=O)N[C@H](C(=O)N[C@@H](C)C(=O)N[C@@H](CCCNC(=N)N)C(*)=O)C1CCCCC1 ; *N[C@@H](Cc1cc(F)c(F)c(F)c1)C(=O)N[C@H](C(=O)N[C@@H](C)C(=O)N[C@@H](CCCNC(=N)N)C(*)=O)C1CCCCC1 ; *N[C@@H](CC1CCCC1)C(=O)N[C@H](C(=O)N[C@@H](C)C(=O)N[C@@H](CCCNC(=N)N)C(*)=O)C1CCCCC1]

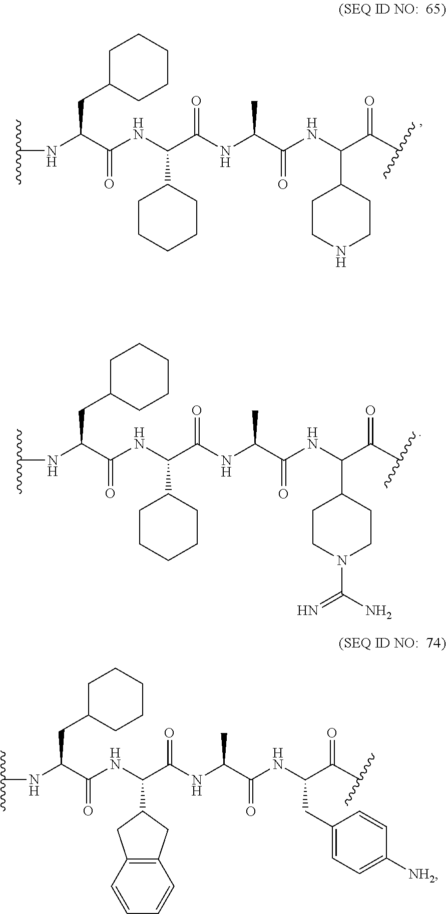 [SMILES: *N[C@@H](CC1CCCCC1)C(=O)N[C@H](C(=O)N[C@@H](C)C(=O)NC(C(*)=O)C1CCNCC1)C1CCCCC1 ; *N[C@@H](CC1CCCCC1)C(=O)N[C@H](C(=O)N[C@@H](C)C(=O)NC(C(*)=O)C1CCN(C(=N)N)CC1)C1CCCCC1 ; *N[C@@H](CC1CCCCC1)C(=O)N[C@H](C(=O)N[C@@H](C)C(=O)N[C@@H](Cc1ccc(N)cc1)C(*)=O)C1Cc2ccccc2C1]

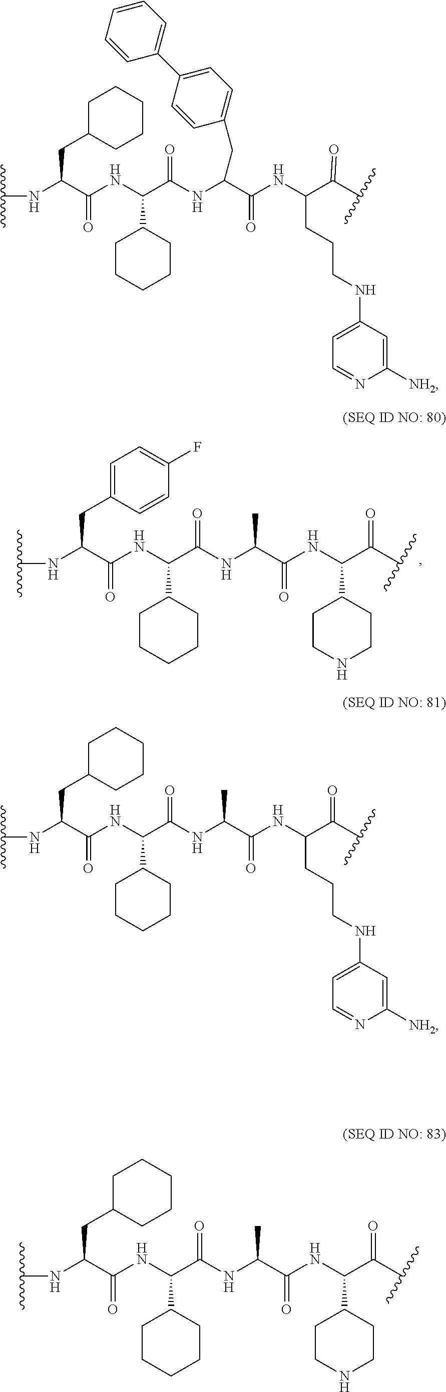 [SMILES: *N[C@@H](CC1CCCCC1)C(=O)N[C@H](C(=O)NC(Cc1ccc(-c2ccccc2)cc1)C(=O)NC(CCCNc1ccnc(N)c1)C(*)=O)C1CCCCC1 ; *N[C@@H](Cc1ccc(F)cc1)C(=O)N[C@H](C(=O)N[C@@H](C)C(=O)N[C@H](C(*)=O)C1CCNCC1)C1CCCCC1 ; *N[C@@H](CC1CCCCC1)C(=O)N[C@H](C(=O)N[C@@H](C)C(=O)NC(CCCNc1ccnc(N)c1)C(*)=O)C1CCCCC1 ; *N[C@@H](CC1CCCCC1)C(=O)N[C@H](C(=O)N[C@@H](C)C(=O)N[C@H](C(*)=O)C1CCNCC1)C1CCCCC1]

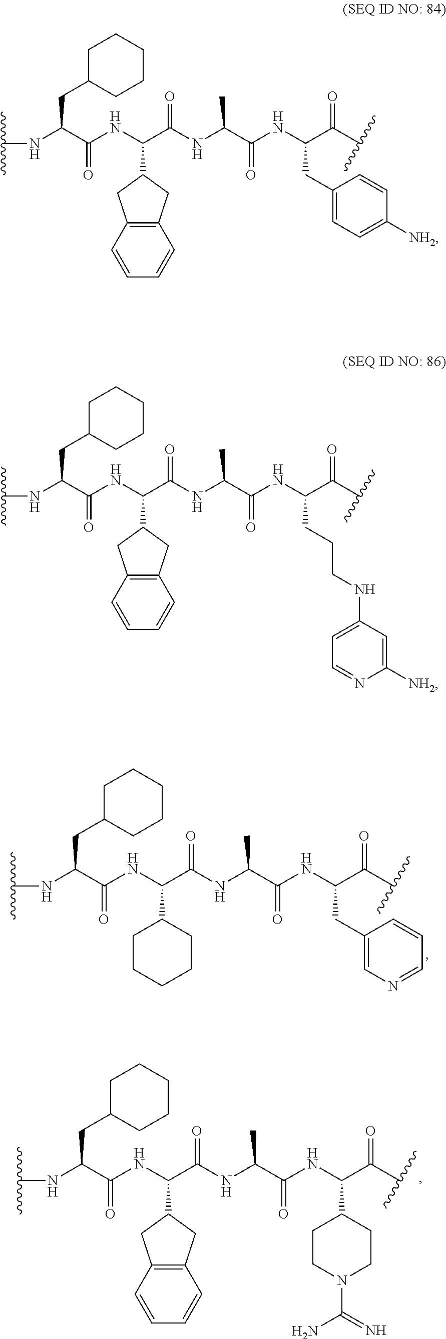 [SMILES: *N[C@@H](CC1CCCCC1)C(=O)N[C@H](C(=O)N[C@@H](C)C(=O)N[C@@H](Cc1ccc(N)cc1)C(*)=O)C1Cc2ccccc2C1 ; *N[C@@H](CC1CCCCC1)C(=O)N[C@H](C(=O)N[C@@H](C)C(=O)N[C@@H](CCCNc1ccnc(N)c1)C(*)=O)C1Cc2ccccc2C1 ; *N[C@@H](CC1CCCCC1)C(=O)N[C@H](C(=O)N[C@@H](C)C(=O)N[C@@H](Cc1cccnc1)C(*)=O)C1CCCCC1 ; *N[C@@H](CC1CCCCC1)C(=O)N[C@H](C(=O)N[C@@H](C)C(=O)N[C@H](C(*)=O)C1CCN(C(=N)N)CC1)C1Cc2ccccc2C1]

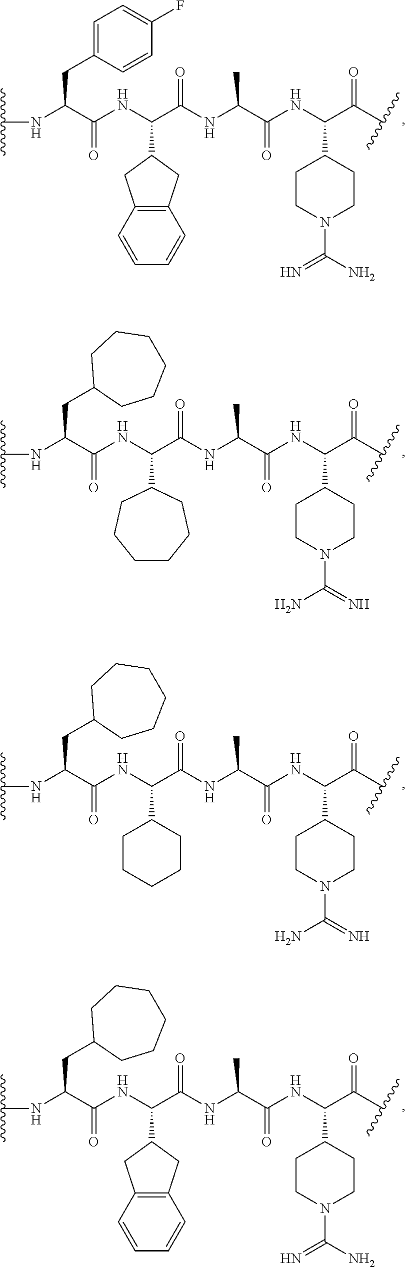 [SMILES: *N[C@@H](Cc1ccc(F)cc1)C(=O)N[C@H](C(=O)N[C@@H](C)C(=O)N[C@H](C(*)=O)C1CCN(C(=N)N)CC1)C1Cc2ccccc2C1 ; *N[C@@H](CC1CCCCCC1)C(=O)N[C@H](C(=O)N[C@@H](C)C(=O)N[C@H](C(*)=O)C1CCN(C(=N)N)CC1)C1CCCCCC1 ; *N[C@@H](CC1CCCCCC1)C(=O)N[C@H](C(=O)N[C@@H](C)C(=O)N[C@H](C(*)=O)C1CCN(C(=N)N)CC1)C1CCCCC1 ; *N[C@@H](CC1CCCCCC1)C(=O)N[C@H](C(=O)N[C@@H](C)C(=O)N[C@H](C(*)=O)C1CCN(C(=N)N)CC1)C1Cc2ccccc2C1]

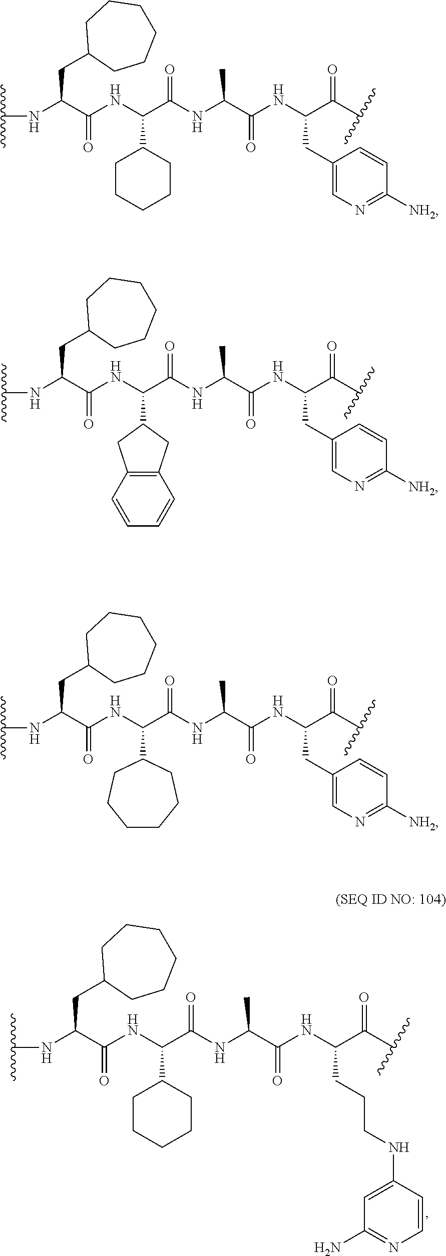 [SMILES: *N[C@@H](CC1CCCCCC1)C(=O)N[C@H](C(=O)N[C@@H](C)C(=O)N[C@@H](Cc1ccc(N)nc1)C(*)=O)C1CCCCC1 ; *N[C@@H](CC1CCCCCC1)C(=O)N[C@H](C(=O)N[C@@H](C)C(=O)N[C@@H](Cc1ccc(N)nc1)C(*)=O)C1Cc2ccccc2C1 ; *N[C@@H](CC1CCCCCC1)C(=O)N[C@H](C(=O)N[C@@H](C)C(=O)N[C@@H](Cc1ccc(N)nc1)C(*)=O)C1CCCCCC1 ; *N[C@@H](CC1CCCCCC1)C(=O)N[C@H](C(=O)N[C@@H](C)C(=O)N[C@@H](CCCNc1ccnc(N)c1)C(*)=O)C1CCCCC1]

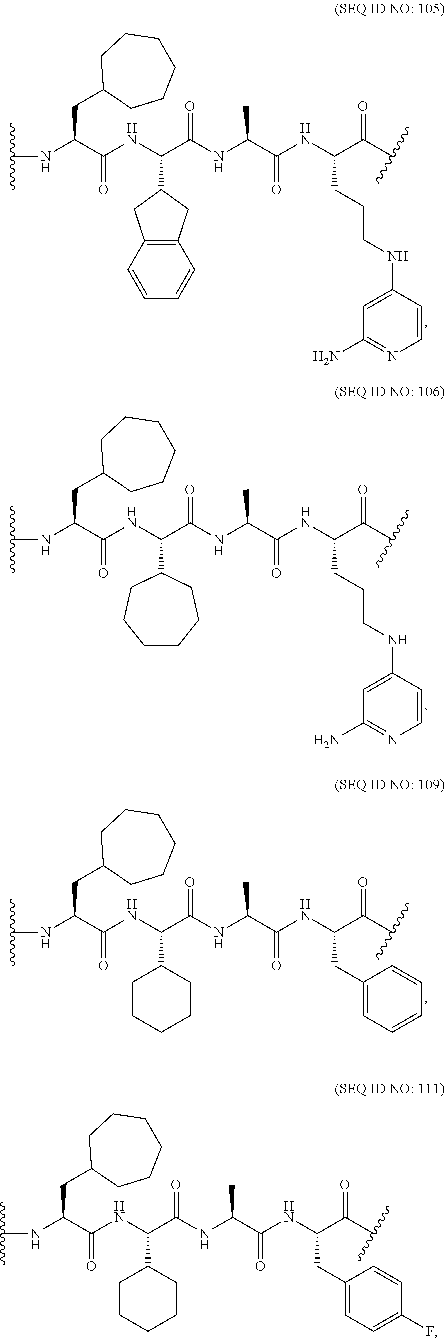 [SMILES: *N[C@@H](CC1CCCCCC1)C(=O)N[C@H](C(=O)N[C@@H](C)C(=O)N[C@@H](CCCNc1ccnc(N)c1)C(*)=O)C1Cc2ccccc2C1 ; *N[C@@H](CC1CCCCCC1)C(=O)N[C@H](C(=O)N[C@@H](C)C(=O)N[C@@H](CCCNc1ccnc(N)c1)C(*)=O)C1CCCCCC1 ; *N[C@@H](CC1CCCCCC1)C(=O)N[C@H](C(=O)N[C@@H](C)C(=O)N[C@@H](Cc1ccccc1)C(*)=O)C1CCCCC1 ; *N[C@@H](CC1CCCCCC1)C(=O)N[C@H](C(=O)N[C@@H](C)C(=O)N[C@@H](Cc1ccc(F)cc1)C(*)=O)C1CCCCC1]

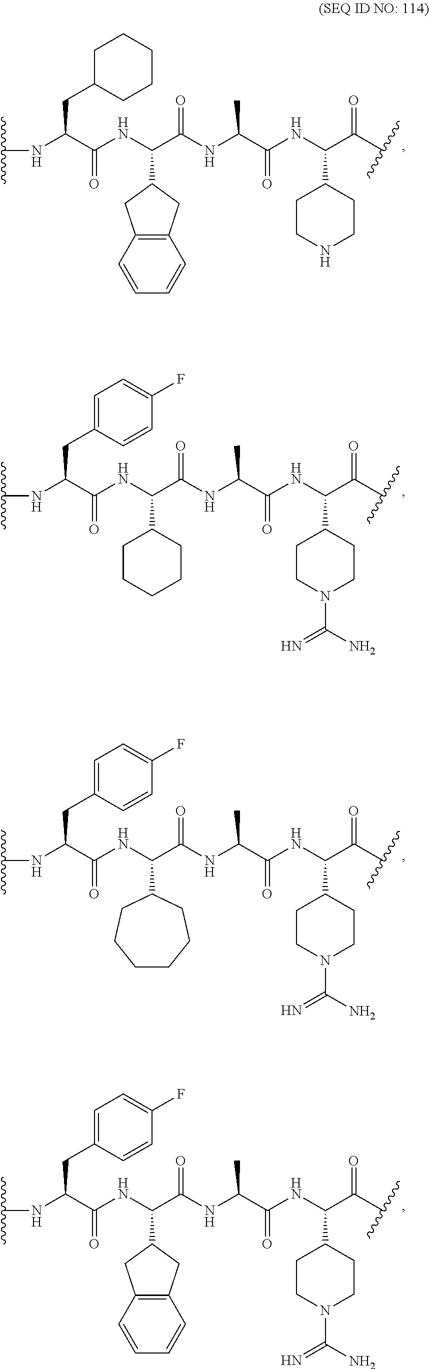 [SMILES: *N[C@@H](CC1CCCCC1)C(=O)N[C@H](C(=O)N[C@@H](C)C(=O)N[C@H](C(*)=O)C1CCNCC1)C1Cc2ccccc2C1 ; *N[C@@H](Cc1ccc(F)cc1)C(=O)N[C@H](C(=O)N[C@@H](C)C(=O)N[C@H](C(*)=O)C1CCN(C(=N)N)CC1)C1CCCCC1 ; *N[C@@H](Cc1ccc(F)cc1)C(=O)N[C@H](C(=O)N[C@@H](C)C(=O)N[C@H](C(*)=O)C1CCN(C(=N)N)CC1)C1CCCCCC1 ; *N[C@@H](Cc1ccc(F)cc1)C(=O)N[C@H](C(=O)N[C@@H](C)C(=O)N[C@H](C(*)=O)C1CCN(C(=N)N)CC1)C1Cc2ccccc2C1]

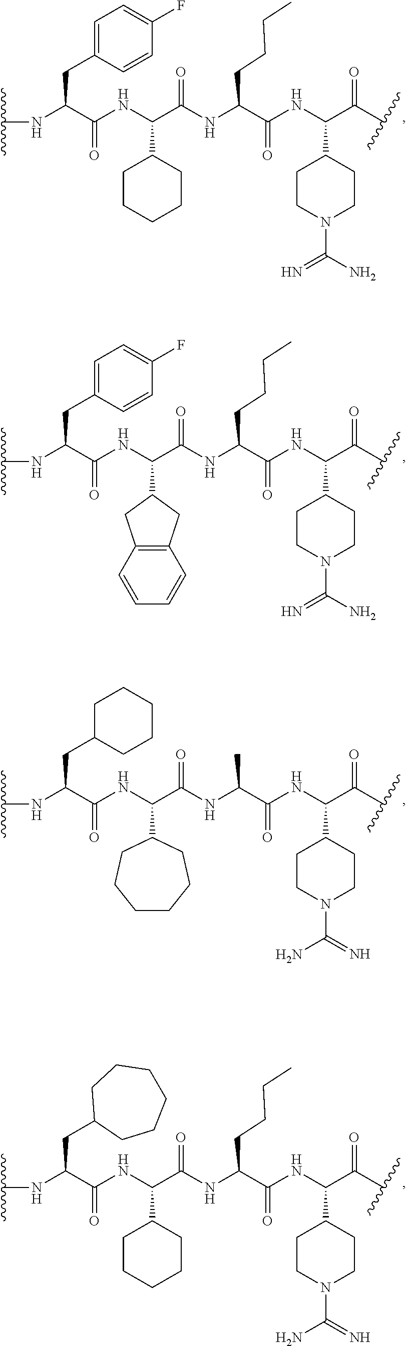 [SMILES: *N[C@@H](Cc1ccc(F)cc1)C(=O)N[C@H](C(=O)N[C@@H](CCCC)C(=O)N[C@H](C(*)=O)C1CCN(C(=N)N)CC1)C1CCCCC1 ; *N[C@@H](Cc1ccc(F)cc1)C(=O)N[C@H](C(=O)N[C@@H](CCCC)C(=O)N[C@H](C(*)=O)C1CCN(C(=N)N)CC1)C1Cc2ccccc2C1 ; *N[C@@H](CC1CCCCC1)C(=O)N[C@H](C(=O)N[C@@H](C)C(=O)N[C@H](C(*)=O)C1CCN(C(=N)N)CC1)C1CCCCCC1 ; *N[C@@H](CC1CCCCCC1)C(=O)N[C@H](C(=O)N[C@@H](CCCC)C(=O)N[C@H](C(*)=O)C1CCN(C(=N)N)CC1)C1CCCCC1]

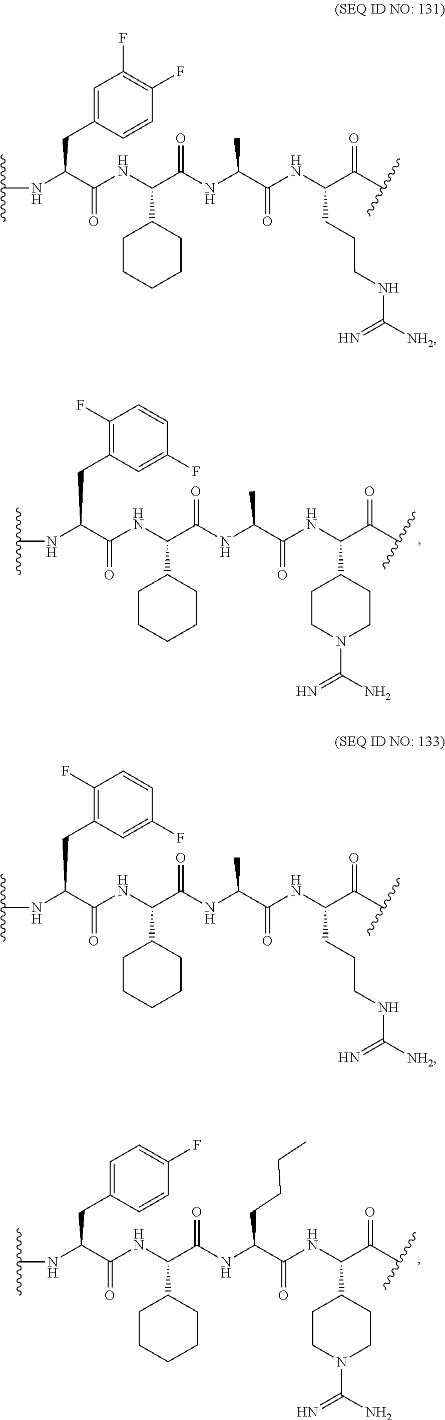 [SMILES: *N[C@@H](Cc1ccc(F)c(F)c1)C(=O)N[C@H](C(=O)N[C@@H](C)C(=O)N[C@@H](CCCNC(=N)N)C(*)=O)C1CCCCC1 ; *N[C@@H](Cc1cc(F)ccc1F)C(=O)N[C@H](C(=O)N[C@@H](C)C(=O)N[C@H](C(*)=O)C1CCN(C(=N)N)CC1)C1CCCCC1 ; *N[C@@H](Cc1cc(F)ccc1F)C(=O)N[C@H](C(=O)N[C@@H](C)C(=O)N[C@@H](CCCNC(=N)N)C(*)=O)C1CCCCC1 ; *N[C@@H](Cc1ccc(F)cc1)C(=O)N[C@H](C(=O)N[C@@H](CCCC)C(=O)N[C@H](C(*)=O)C1CCN(C(=N)N)CC1)C1CCCCC1]

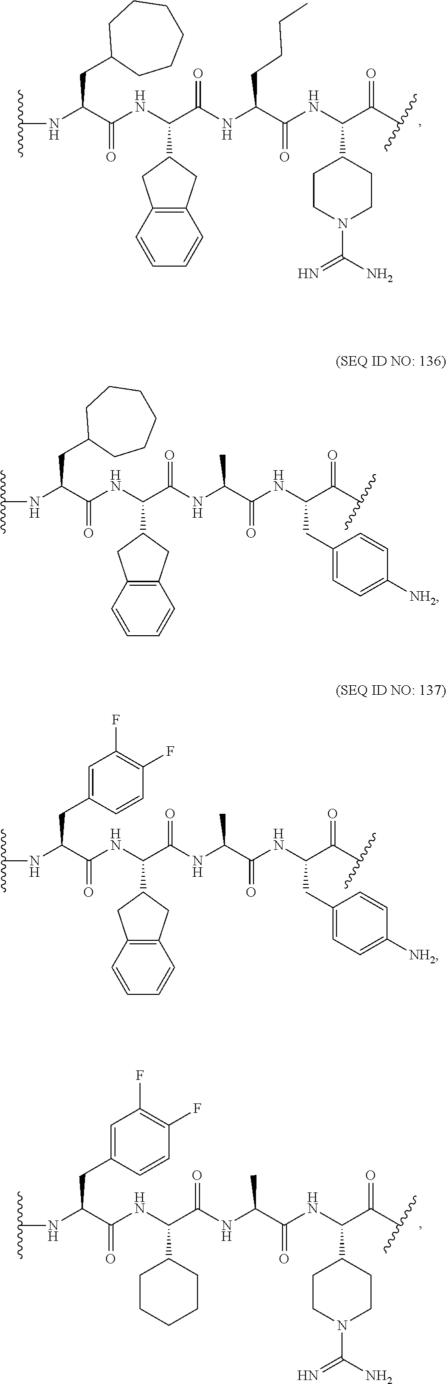 [SMILES: *N[C@@H](CC1CCCCCC1)C(=O)N[C@H](C(=O)N[C@@H](CCCC)C(=O)N[C@H](C(*)=O)C1CCN(C(=N)N)CC1)C1Cc2ccccc2C1 ; *N[C@@H](CC1CCCCCC1)C(=O)N[C@H](C(=O)N[C@@H](C)C(=O)N[C@@H](Cc1ccc(N)cc1)C(*)=O)C1Cc2ccccc2C1 ; *N[C@@H](Cc1ccc(F)c(F)c1)C(=O)N[C@H](C(=O)N[C@@H](C)C(=O)N[C@@H](Cc1ccc(N)cc1)C(*)=O)C1Cc2ccccc2C1 ; *N[C@@H](Cc1ccc(F)c(F)c1)C(=O)N[C@H](C(=O)N[C@@H](C)C(=O)N[C@H](C(*)=O)C1CCN(C(=N)N)CC1)C1CCCCC1]

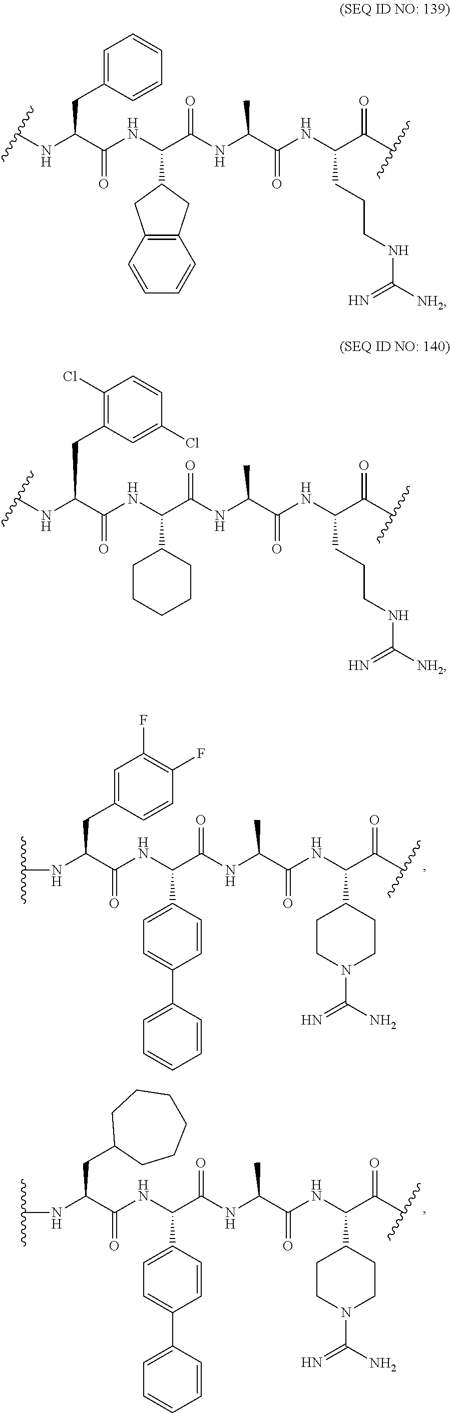

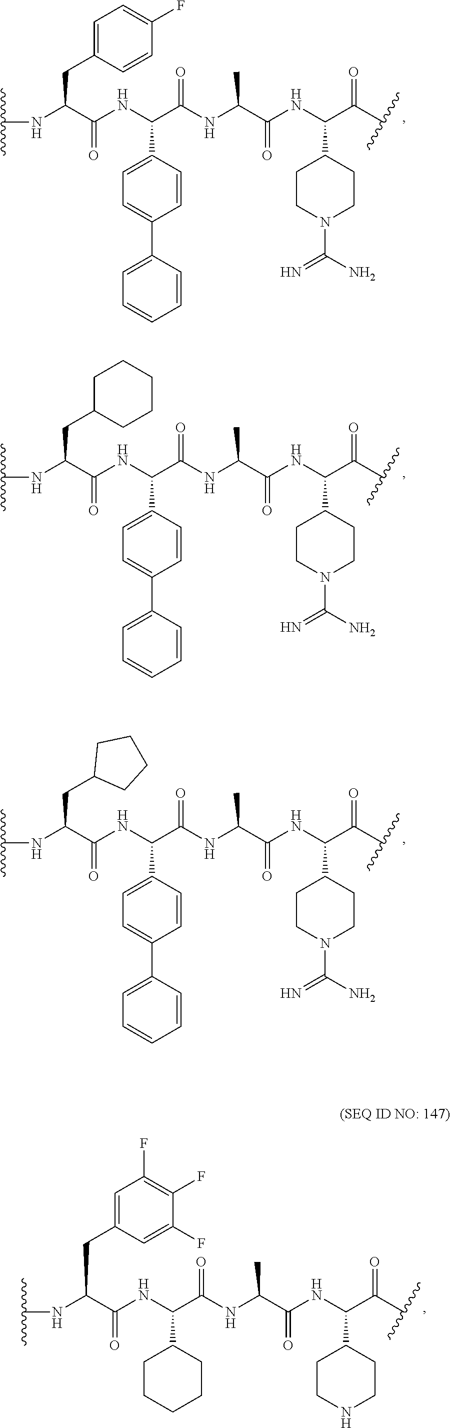 [SMILES: *N[C@@H](Cc1ccc(F)cc1)C(=O)N[C@H](C(=O)N[C@@H](C)C(=O)N[C@H](C(*)=O)C1CCN(C(=N)N)CC1)c1ccc(-c2ccccc2)cc1 ; *N[C@@H](CC1CCCCC1)C(=O)N[C@H](C(=O)N[C@@H](C)C(=O)N[C@H](C(*)=O)C1CCN(C(=N)N)CC1)c1ccc(-c2ccccc2)cc1 ; *N[C@@H](CC1CCCC1)C(=O)N[C@H](C(=O)N[C@@H](C)C(=O)N[C@H](C(*)=O)C1CCN(C(=N)N)CC1)c1ccc(-c2ccccc2)cc1 ; *N[C@@H](Cc1cc(F)c(F)c(F)c1)C(=O)N[C@H](C(=O)N[C@@H](C)C(=O)N[C@H](C(*)=O)C1CCNCC1)C1CCCCC1]

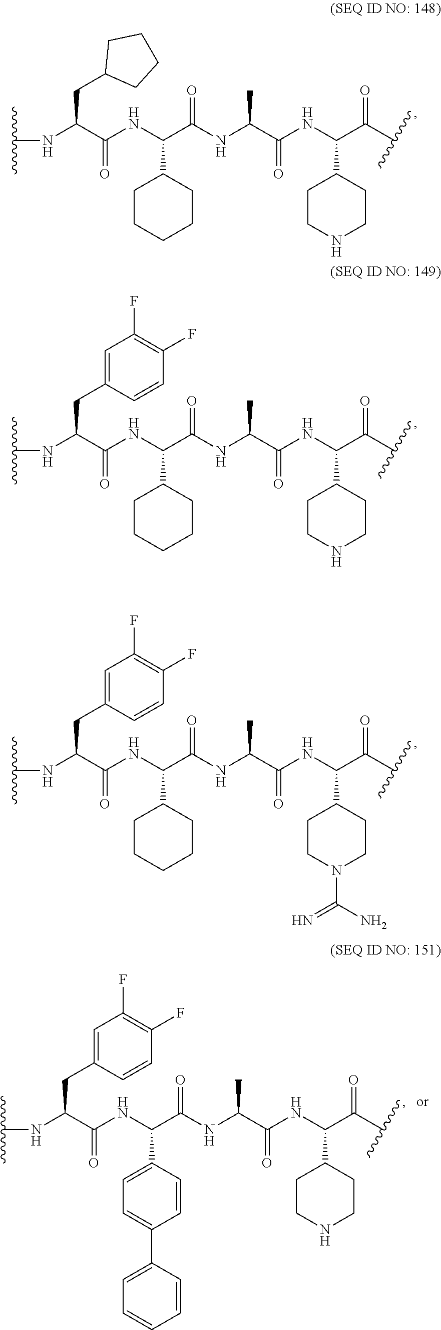 [SMILES: *N[C@@H](CC1CCCC1)C(=O)N[C@H](C(=O)N[C@@H](C)C(=O)N[C@H](C(*)=O)C1CCNCC1)C1CCCCC1 ; *N[C@@H](Cc1ccc(F)c(F)c1)C(=O)N[C@H](C(=O)N[C@@H](C)C(=O)N[C@H](C(*)=O)C1CCNCC1)C1CCCCC1 ; *N[C@@H](Cc1ccc(F)c(F)c1)C(=O)N[C@H](C(=O)N[C@@H](C)C(=O)N[C@H](C(*)=O)C1CCN(C(=N)N)CC1)C1CCCCC1 ; *N[C@@H](Cc1ccc(F)c(F)c1)C(=O)N[C@H](C(=O)N[C@@H](C)C(=O)N[C@H](C(*)=O)C1CCNCC1)c1ccc(-c2ccccc2)cc1]

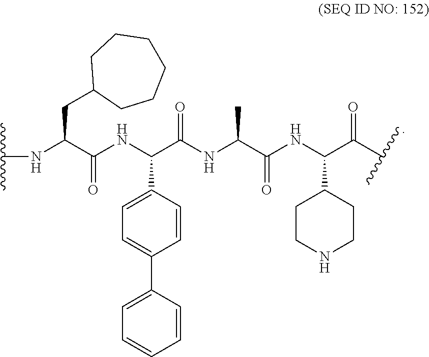 [SMILES: *N[C@@H](CC1CCCCCC1)C(=O)N[C@H](C(=O)N[C@@H](C)C(=O)N[C@H](C(*)=O)C1CCNCC1)c1ccc(-c2ccccc2)cc1]

In some embodiments, R^{a}—X¹—X²—X³—X⁴— is:

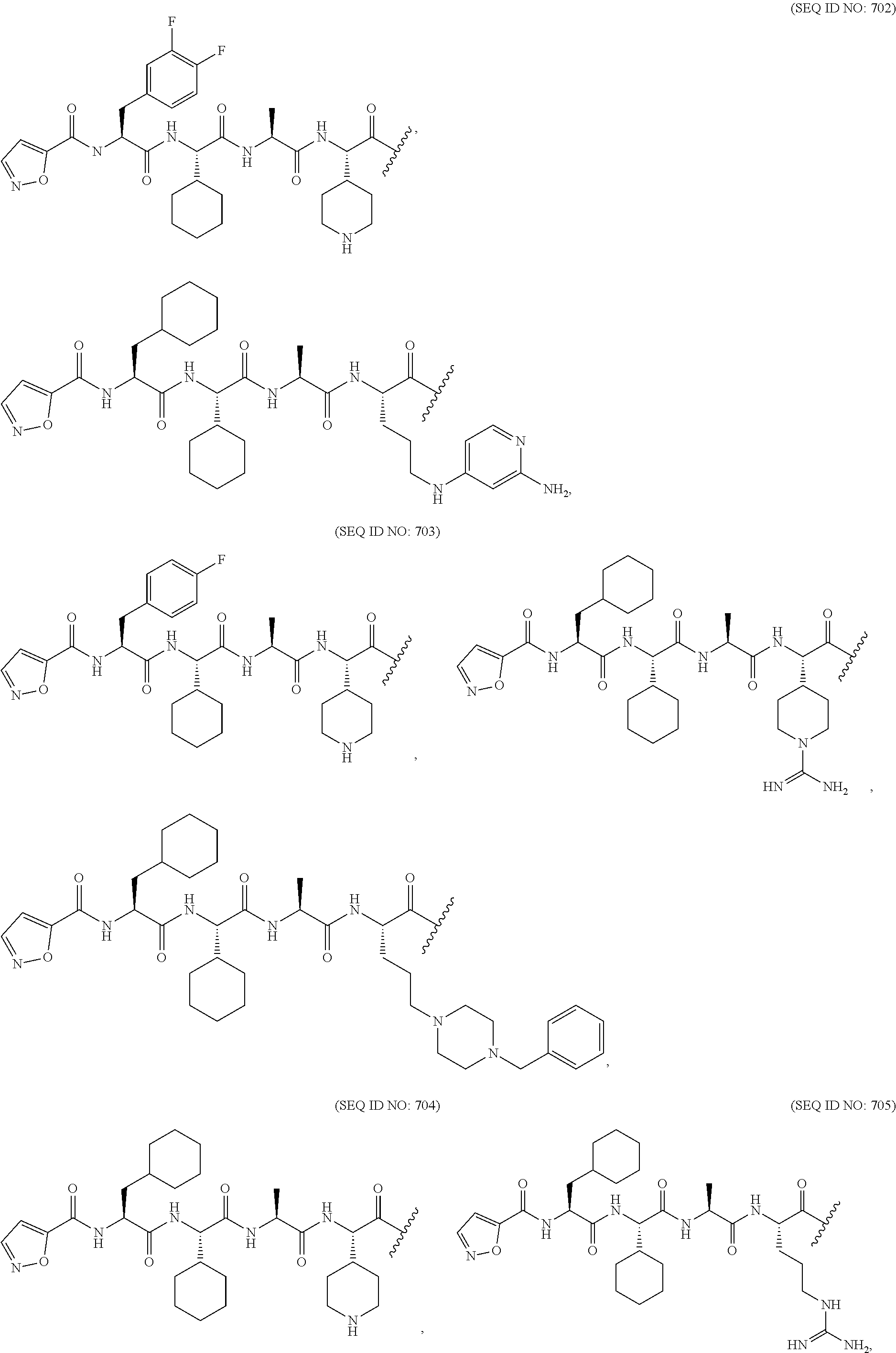

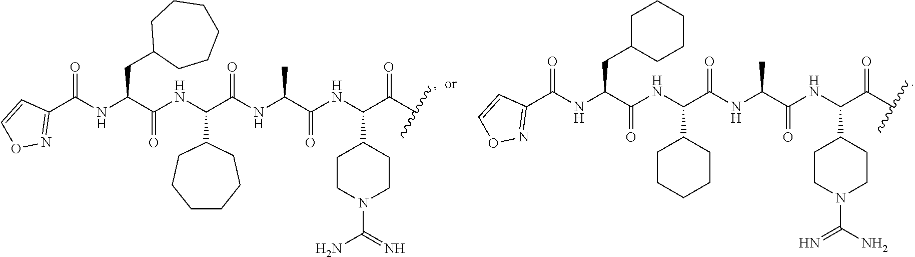 [SMILES: *C(=O)[C@@H](NC(=O)[C@H](C)NC(=O)[C@@H](NC(=O)[C@H](CC1CCCCCC1)NC(=O)c1ccon1)C1CCCCCC1)C1CCN(C(=N)N)CC1 ; *C(=O)[C@@H](NC(=O)[C@H](C)NC(=O)[C@@H](NC(=O)[C@H](CC1CCCCC1)NC(=O)c1ccon1)C1CCCCC1)C1CCN(C(=N)N)CC1]

In some embodiments, X⁵ is -L⁵-, (S)-alanine (Ala), (S)-lysine (Lys), (S)-ornithine (Orn), N6-palmitoyl-(S)-lysine, N6-(15-carboxypentadecanoyl)-(S)-Lys, N6-(16-oxo-16-((2-(N—N—N-trimethyl-amino)ethyl)amino)hexadecanoyl)-Lys, N6-(4-fluorobenzoyl)-(S)-lysine, (S)-β³-homolysine, (S)-3-amino-alanine (H-Dap-OH), 4-fluoro-(S)-phenylalanine (Phe(4-F)), 4-iodo-(S)-phenylalanine (Phe(4-1)), 4-nitro-(S)-phenylalanine (Phe(4-NO₂)), (S)-(4-fluorobenzamido)methylglycine, 4-aminomethyl-(S)-phenylalanine (Phe(4-CH₂—NH₂)), (S)-7-hydroxy-1,2,3,4-tetrahydroisoquinoline-3-carboxylic acid, (S)-proline, (S)-hydroxyproline (Hyp) (S)-piperidine-2-carboxylic acid, (S)-isoindoline-1-carboxylic acid, or (S)-(4-(aminomethyl)-1H-1,2λ²,3λ²-triazol-1-yl)methyl)-glycine.

In some embodiments, X⁵ is N6-palmitoyl-(S)-lysine, N6-(15-carboxypentadecanoyl)-(S)-Lys, N6-(16-oxo-16-((2-(N—N—N-trimethyl-amino)ethyl)amino)hexadecanoyl)-(S)-Lys, N6-(4-fluorobenzoyl)-(S)-lysine, (S)-β³-homolysine, (S)-3-amino-alanine (H-Dap-OH), 4-fluoro-(S)-phenylalanine (Phe(4-F)), 4-iodo-(S)-phenylalanine (Phe(4-I)), 4-nitro-(S)-phenylalanine (Phe(4-NO₂)), (S)-(4-fluorobenzamido)methylglycine, 4-amidomethyl-(S)-phenylalanine (Phe(4-CH₂NH₂)), (S)-7-hydroxy-1,2,3,4-tetrahydroisoquinoline-3-carboxylic acid, (S)-proline, (S)-hydroxyproline (Hyp), (S)-piperidine-2-carboxylic acid, (S)-isoindoline-1-carboxylic acid, or (S)-(4-(aminomethyl)-1H-1,2λ²,3λ²-triazol-1-yl)methyl)-glycine.

In some embodiments, X⁵ is absent. In some embodiments, X⁵ is -L⁵-. In some embodiments, X⁵ is alanine (Ala). In some embodiments, X⁵ is lysine (Lys). In some embodiments, X⁵ is ornithine (Orn). In some embodiments, X⁵ is β³-homolysine. In some embodiments, X⁵ is γ-glutamic acid (γ-Glu). In some embodiments, X⁵ is leucine (Leu). In some embodiments, X⁵ is phenylalanine (Phe). In some embodiments, X⁵ is 4-azidophenylalanine (Phe(4-N₃)). In some embodiments, X⁵ is norleucine (Nle). In some embodiments, X⁵ is 2-aminoheptanoic acid. In some embodiments, X⁵ is 2-amino-6-azidohexanoic acid. In some embodiments, X⁵ is N6-acetyl-lysine. In some embodiments, X⁵ is N6-(pent-4-ynoyl)-lysine. In some embodiments, X⁵ is N6-octanoyl-lysine. In some embodiments, X⁵ is tyrosine (Tyr). In some embodiments, X⁵ is N6-palmitoyl-lysine. In some embodiments, X⁵ is N6-(15-carboxypentadecanoyl)-Lys. In some embodiments, X⁵ is N6-(16-oxo-16-((2-(N—N—N-trimethyl-amino)ethyl)amino)hexadecanoyl)-Lys. In some embodiments, X⁵ is N6-(4-fluorobenzoyl)-lysine. In some embodiments, X⁵ is β³-homolysine. In some embodiments, X⁵ is 3-amino-alanine (H-Dap-OH). In some embodiments, X⁵ is 4-fluoro-phenylalanine (Phe(4-F)). In some embodiments, X⁵ is 4-iodo-phenylalanine (Phe(4-I)). In some embodiments, X⁵ is 4-nitro-phenylalanine (Phe(4-N02)) In some embodiments, X⁵ is (4-fluorobenzamido)methylglycine. In some embodiments, X⁵ is 4-aminomethylphenylalanine (Phe(4-CH₂NH₂)). In some embodiments, X⁵ is 4-azidomethylphenylalanine (Phe(4-CH₂N₃)). In some embodiments, X⁵ is 4-amino-1-((3-fluoro-4-methylbenzyl)glycyl)piperidine-4-carboxylic acid. In some embodiments, X⁵ is 7-hydroxy-1,2,3,4-tetrahydroisoquinoline-3-carboxylic acid. In some embodiments, X⁵ is proline. In some embodiments, X⁵ is hydroxyproline (Hyp). In some embodiments, X⁵ is piperidine-2-carboxylic acid. In some embodiments, X⁵ is isoindoline-1-carboxylic acid. In some embodiments, 5 is (4-(aminomethyl)-1H-1,2λ²,3λ²-triazol-1-yl)methyl)-glycine.

In some embodiments, X⁵ is alanine (Ala), lysine (Lys), ornithine (Orn),

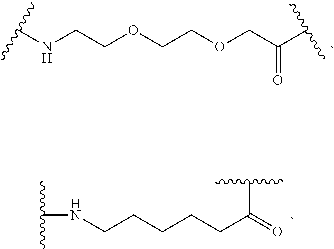 [SMILES: *NCCOCCOCC(*)=O ; *NCCCCCC(*)=O]

N6-(4-fluorobenzoyl)-Lys, β³-homolysine, 3-amino-alanine (H-Dap-OH), 4-nitrophenylalanine (Phe(4-NO₂)), or (4-fluorobenzamido)methylglycine.

In some embodiments, X⁵ is -L⁵-, (S)-alanine (ala), (S)-lysine (Lys), (S)-ornithine (Orn), or (S)-(4-fluorobenzamido)methylglycine. In some embodiments, X⁵ is (S)-lysine (Lys) or (S)-ornithine (Orn). In some embodiments, X⁵ is (R)-lysine (Lys) or (R)-ornithine (Orn). In some embodiments, X⁵ is -L⁵-. In some embodiments, X⁵ is (S)-alanine (ala). In some embodiments, X⁵ is (S)-lysine (Lys). In some embodiments, X⁵ is (S)-ornithine (Orn). In some embodiments, X⁵ is (R)-lysine (Lys). In some embodiments, X⁵ is (R)-ornithine (Orn). In some embodiments, X⁵ is (S)-(4-fluorobenzamido)methylglycine.

In some embodiments, X⁵ is absent or

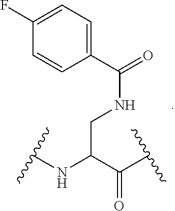 [SMILES: *NC(CNC(=O)c1ccc(F)cc1)C(*)=O]

In some embodiments,
X¹ is

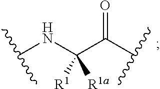

R¹ is -L¹-R^{1b}; L¹ is —CH₂—; R^{1b} is cyclohexyl, cycloheptyl, or 4-fluorophenyl; R^{1a} is hydrogen;
X² is

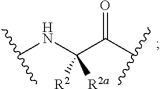

R² is -L²-R^{2b}; L² is absent; R^{2b} is cyclohexyl, cycloheptyl, 2,3-dihydro-1H-inden-2-yl, or [1,1′-biphenyl]-4-yl; R^{2a} is hydrogen;
X³ is (S)-alanine (Ala) or (S)-norleucine (Nle);
X⁴ is (S)-arginine (Arg), (S)-ornithine (Orn), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-3-(6-aminopyridin-3-yl)alanine ((3-Pal(6-NH₂)), or (S)-2-((2-aminopyridin-4-yl)-λ²-azaneyl)ethyl alanine; and
X⁵ is -L⁵-, alanine (Ala), lysine (Lys), or ornithine (Orn).

In some embodiments, X⁶ is absent, lysine (Lys), ornithine (Orn),

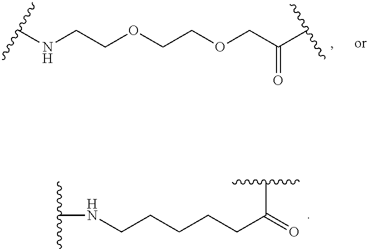 [SMILES: *NCCOCCOCC(*)=O ; *NCCCCCC(*)=O]

In some embodiments, X⁶ is absent, -L⁶-, lysine (Lys), or ornithine (Orn). In some embodiments, X⁶ is absent. In some embodiments, X⁶ is -L⁶-. In some embodiments, X⁶ is (S)-lysine (Lys) or (S)-ornithine (Orn). In some embodiments, X⁶ is (R)-lysine (Lys) or (R)-ornithine (Gm). In some embodiments, X⁶ is (S)-lysine (Lys). In some embodiments, X⁶ is (S)-ornithine (Orn). In some embodiments, X⁶ is (R)-lysine (Lys). In some embodiments, X⁶ is (R)-ornithine (Orn). In some embodiments, X⁶ is

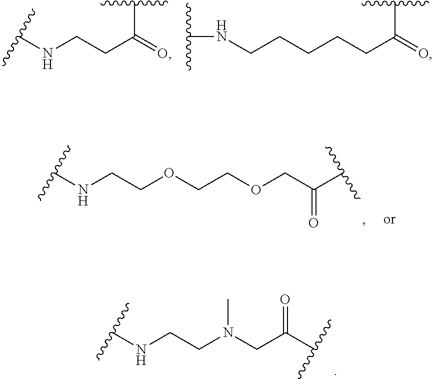

In some embodiments, X⁶ is

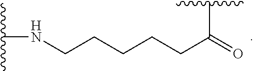 [SMILES: *NCCCCCC(*)=O]

In some embodiments, X⁶ is

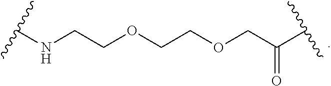 [SMILES: *NCCOCCOCC(*)=O]

In some embodiments, X⁶ is

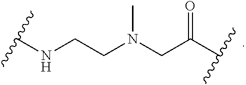 [SMILES: *NCCN(C)CC(*)=O]

In some embodiments, X⁶ is 4-amino-1-(methylglycyl)piperidine-4-carboxylic acid, or 4-amino-1-(N-carbamimidoyl-N-methylglycyl)piperidine-4-carboxylic acid. In some embodiments, X⁶ is (4-fluorobenzamido)methylglycine. In some embodiments, X⁶ is N6-(4-fluorobenzoyl)-Lys.

In some embodiments, X⁷ is absent, lysine (Lys), ornithine (Orn), or

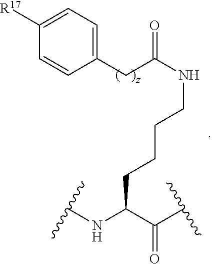

In some embodiments, X⁷ is absent. In some embodiments, X⁷ is -L⁷-. In some embodiments, X⁷ is (S)-lysine (Lys) or (S)-ornithine (Orn). In some embodiments, X⁷ is (S)-lysine (Lys). In some embodiments, X⁷ is (S)-ornithine (Orn). In some embodiments, X⁷ is glutamic acid (Glu). In some embodiments, X⁷ is norleucine (Nle). In some embodiments. X⁷ is

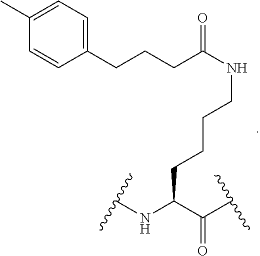 [SMILES: *N[C@@H](CCCCNC(=O)CCCc1ccc(C)cc1)C(*)=O]

In some embodiments, X⁷ is

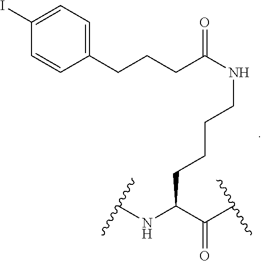 [SMILES: *N[C@@H](CCCCNC(=O)CCCc1ccc(I)cc1)C(*)=O]

In some embodiments, X⁷ is

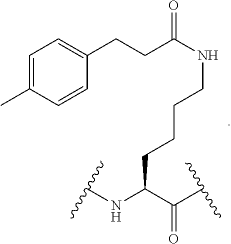 [SMILES: *N[C@@H](CCCCNC(=O)CCc1ccc(C)cc1)C(*)=O]

In some embodiments, X⁷ is

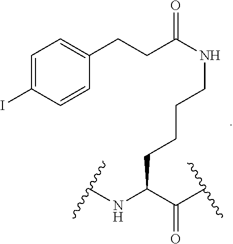 [SMILES: *N[C@@H](CCCCNC(=O)CCc1ccc(I)cc1)C(*)=O]

In some embodiments, X⁷ is

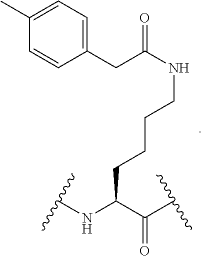 [SMILES: *N[C@@H](CCCCNC(=O)Cc1ccc(C)cc1)C(*)=O]

In some embodiments, X⁷ is

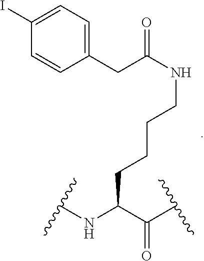 [SMILES: *N[C@@H](CCCCNC(=O)Cc1ccc(I)cc1)C(*)=O]

In some embodiments, X⁸ is absent. In some embodiments, X⁸ is lysine (Lys). In some embodiments, X⁸ is glutamic acid (Glu).

In some embodiments, R^{a} is —C(═O)—R¹³, wherein R¹⁰ is a heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl, indolizinyl, indolyl, benzofuranyl, benzothiophenyl, indazolyl, benzimidazolyl, purinyl, quinolizinyl, quinolinyl, isoquinolinyl, cinnolinyl, phthalazinyl, quinazolinyl, quinoxalinyl, 1,8-naphthyridinyl, 1,7-naphthyridinyl, 1,6-naphthyridinyl, 1,5-naphthyridinyl, pteridinyl, and tetrazolo[1,5-a]pyridinyl; and wherein R⁰ is optionally substituted with 1 to 3 substituents that are each independently selected from the group consisting of halo, C₁-C₆ alkyl, alkoxy, —OH, —NH₂, —NH—C₁-C₆ alkylene-R¹¹, and —O—C₁-C₆ alkylene-R¹. In some embodiments, R^{a} is —C(═O)—R¹⁰, wherein R¹⁰ is a monocyclic heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl; and wherein R¹⁰ is optionally substituted with 1 to 3 substituents that are each independently selected from the group consisting of halo, C₁-C₆ alkyl, alkoxy, —OH, —NH₂, —NH—C₁-C₆ alkylene-R¹¹, and —O—C₁-C₆ alkylene-R¹¹. In some embodiments, R^{a} is —C(═O)—R¹⁰, wherein R¹⁰ is a monocyclic heteroaryl selected from the group consisting of pyridinyl, pyrimidinyl, pyrazinyl, and pyridazinyl; and wherein R¹⁰ is optionally substituted with —NH—C₁-C₆ alkylene-R¹¹ or —O—C₁-C₆ alkylene-R¹¹. In some embodiments, R^{a} is —C(═O)—R¹⁰, wherein R¹⁰ is a phenyl or naphthyl; and wherein R¹⁰ is optionally substituted with 1 to 3 substituents that are each independently selected from the group consisting of halo, C₁-C₆ alkyl, alkoxy, —OH, —NH₂, —NH—C₁-C₆alkylene-R¹¹, and —O—C₁-C₆ alkylene-R¹¹. In some embodiments, R^{a} is —C(═O)—R¹⁰, wherein R¹⁰ is a partially unsaturated bicyclic heterocyclic ring; wherein any saturated carbon of R¹⁰ is optionally substituted with oxo (═O); and wherein R¹⁰ is optionally substituted with 1 to 3 substituents that are each independently selected from the group consisting of halo, C₁-C₆ alkyl, alkoxy, —OH, and —NH₂. In some embodiments, R^{a} is —C(═O)—R¹⁰, wherein R¹⁰ is unsubstituted isoxazolyl.

In some embodiments, R¹⁰ is pyridinyl. In some embodiments, R¹¹ is imidazolyl. In some embodiments, R¹¹ is pyrimidinyl. In some embodiments, R¹⁰ is pyrazolyl. In some embodiments, R¹⁰ is triazolyl. In some embodiments, R¹⁰ is pyrazinyl. In some embodiments, R¹⁰ is tetrazolyl. In some embodiments, R¹⁰ is furyl. In some embodiments, R¹¹ is thienyl. In some embodiments, R¹¹ is isoxazolyl. In some embodiments, R¹¹ is thiazolyl. In some embodiments, R¹⁰ is oxazolyl. In some embodiments, R¹⁰ is isothiazolyl. In some embodiments, R¹⁰ is pyrrolyl. In some embodiments, R¹⁰ is pyridazinyl. In some embodiments, R¹⁰ is triazinyl. In some embodiments, R¹⁰ is oxadiazolyl. In some embodiments, R¹¹ is thiadiazolyl. In some embodiments, R¹⁰ is furazanyl. In some embodiments, R¹¹ is indolizinyl. In some embodiments, R¹¹ is indolyl. In some embodiments, R¹⁰ is benzofuranyl. In some embodiments, R¹⁰ is benzothiophenyl. In some embodiments, R¹⁰ is indazolyl. In some embodiments, R¹⁰ is benzimidazolyl. In some embodiments, R¹¹ is purinyl. In some embodiments, R¹⁰ is quinolizinyl. In some embodiments, R¹⁰ is quinolinyl. In some embodiments, R¹⁰ is isoquinolinyl. In some embodiments, R¹⁰ is cinnolinyl. In some embodiments, R¹⁰ is phthalazinyl. In some embodiments, R¹⁰ is quinazolinyl. In some embodiments, R¹⁰ is quinoxalinyl. In some embodiments, R¹¹ is 1,8-naphthyridinyl. In some embodiments, R¹⁰ is 1,7-naphthyridinyl. In some embodiments, R¹⁰ is 1,6-naphthyridinyl. In some embodiments, R¹⁰ is 1,5-naphthyridinyl. In some embodiments, R¹⁰ is pteridinyl. In some embodiments, R¹⁰ is tetrazolo[1,5-a]pyridinyl. In the aforementioned embodiments, R¹⁰ is substituted with 1 to 3 halo, C₁-C₆ alkyl, alkoxy, —OH, —NH₂, —NH—CH₂—R¹¹, —O—CH₂—R¹¹, —NH—CH₂CH₂—R¹¹, —O—CH₂CH₂—R¹¹, —NH—CH₂CH₂CH₂—R¹¹, —O—CH₂CH₂CH₂—R¹¹, —NH—CH₂CH₂CH₂CH₂—R¹¹, or —O—CH₂CH₂CH₂CH₂—R¹¹.

In some embodiments, R¹¹ is unsubstituted phenyl. In some embodiments, R¹¹ is phenyl substituted with 1, 2 or 3 substituents selected from the group consisting of: —F, —Cl, —Br, —CH₃, —CF₃, —OCH₃, —NH₂, or —NO₂. In some embodiments, R¹¹ is unsubstituted pyridinyl. In some embodiments, R¹¹ is unsubstituted isoindolinyl.

In some embodiments, R^{a} is

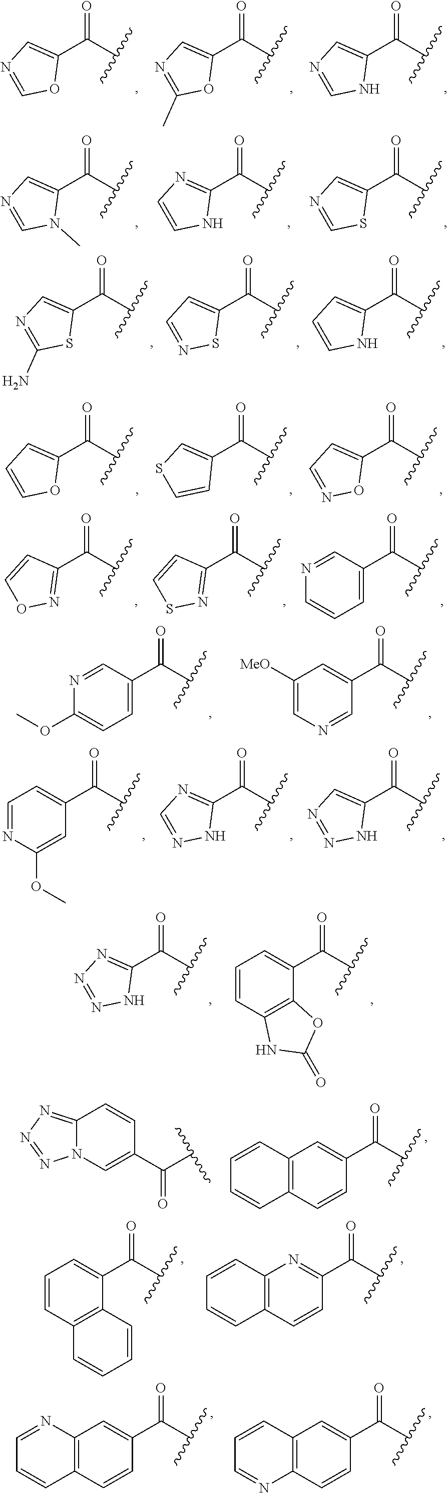

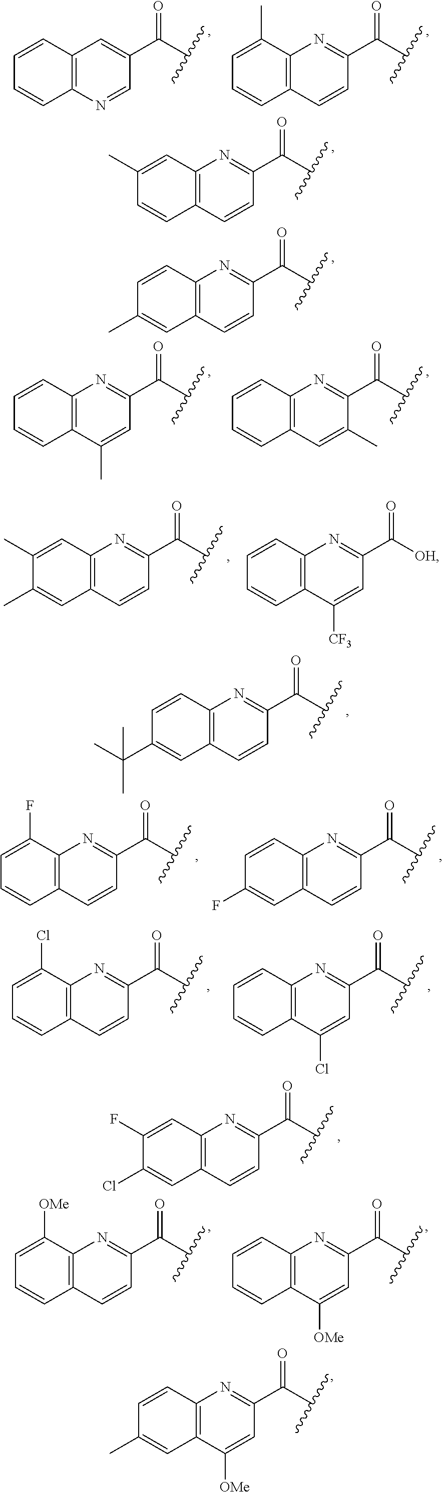

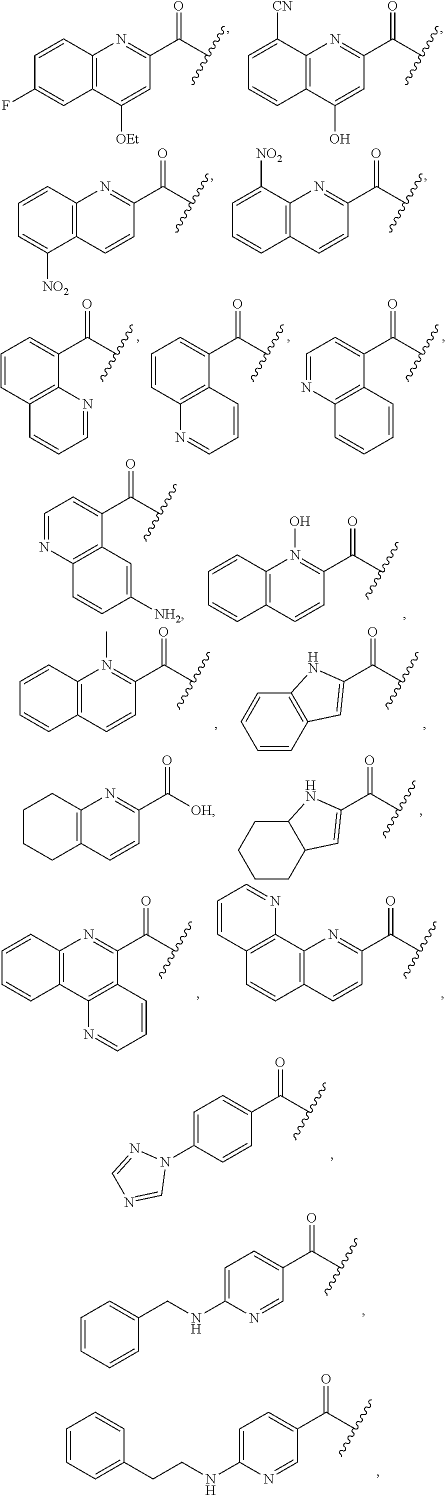

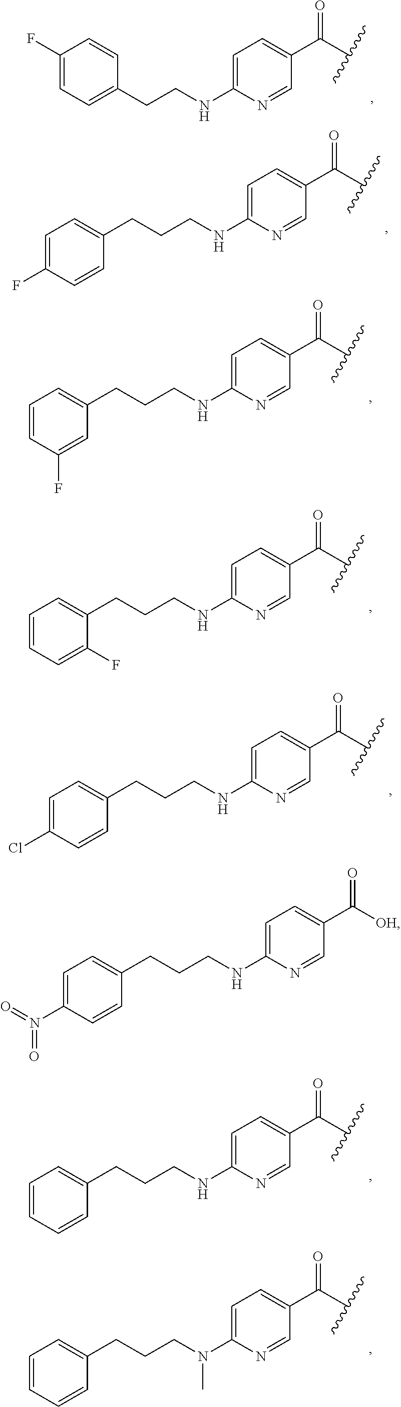

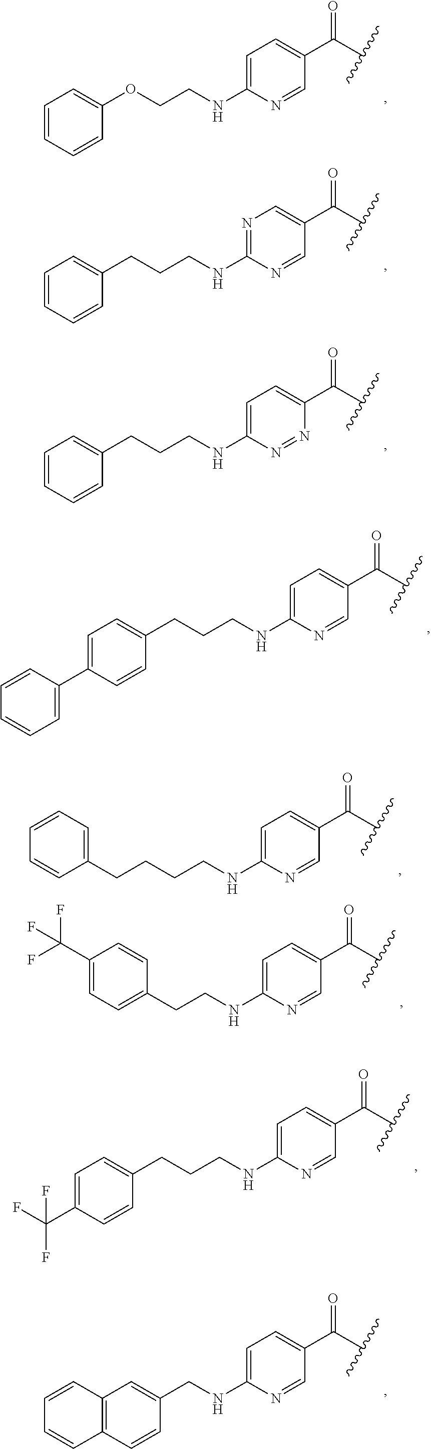

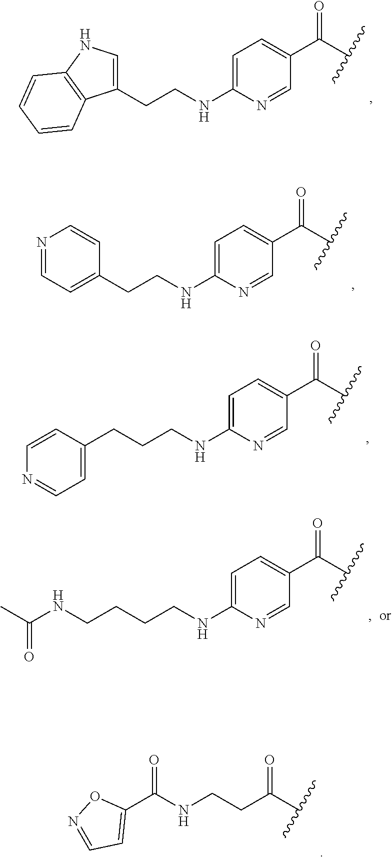 [SMILES: *C(=O)c1ccc(NCCc2c[nH]c3ccccc23)nc1 ; *C(=O)c1ccc(NCCc2ccncc2)nc1 ; *C(=O)c1ccc(NCCCc2ccncc2)nc1 ; *C(=O)c1ccc(NCCCCNC(C)=O)nc1 ; *C(=O)CCNC(=O)c1ccno1]

In some embodiments, R^{a} is

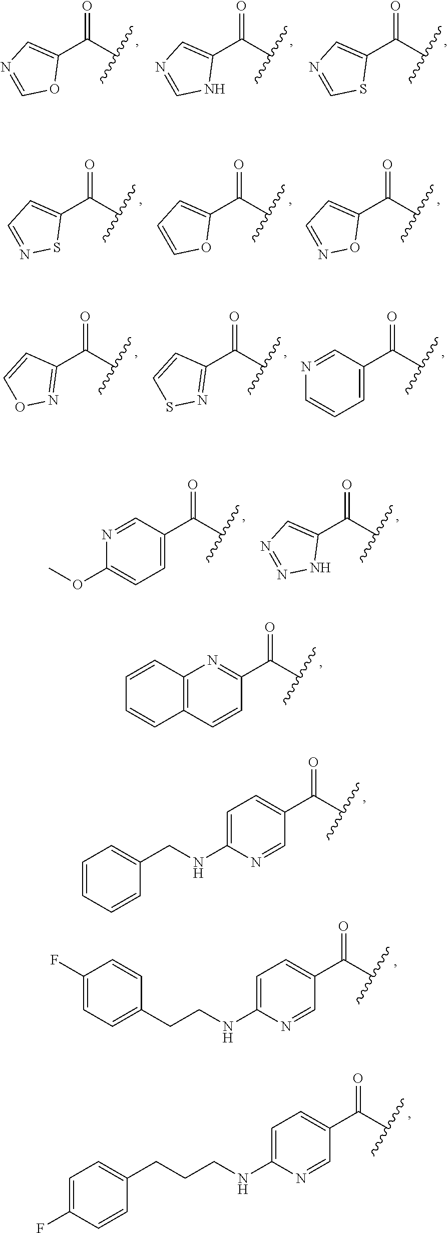

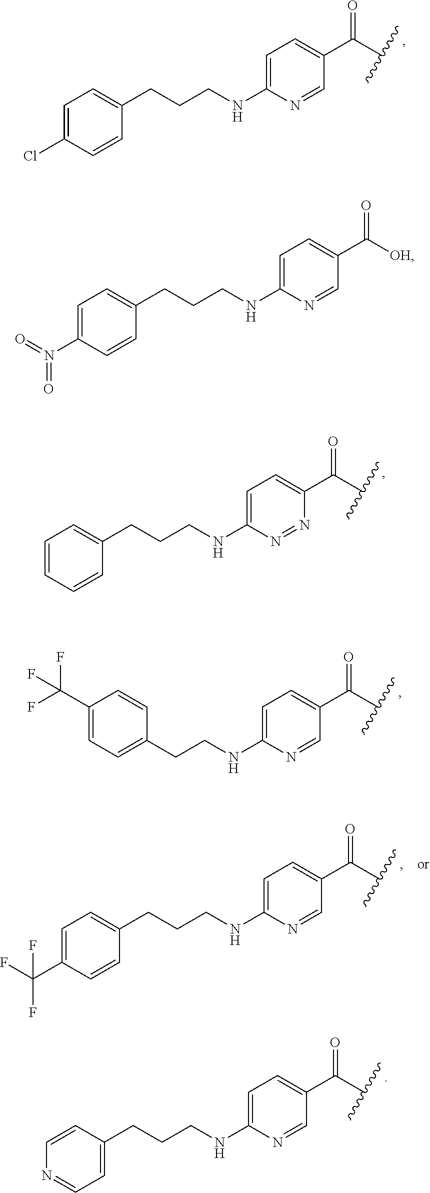 [SMILES: *C(=O)c1ccc(NCCCc2ccc(Cl)cc2)nc1 ; O=C(O)c1ccc(NCCCc2ccc([N+](=O)[O-])cc2)nc1 ; *C(=O)c1ccc(NCCCc2ccccc2)nn1 ; *C(=O)c1ccc(NCCc2ccc(C(F)(F)F)cc2)nc1 ; *C(=O)c1ccc(NCCCc2ccc(C(F)(F)F)cc2)nc1 ; *C(=O)c1ccc(NCCCc2ccncc2)nc1]

In some embodiments, R^{a} is

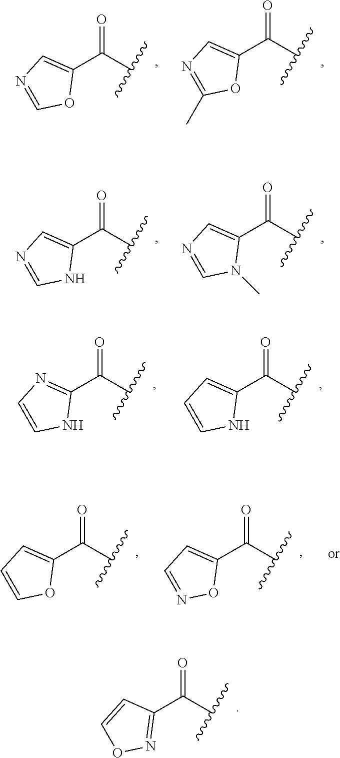

In some embodiments, R^{a} is

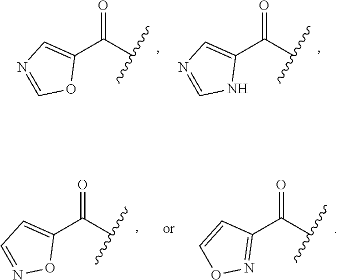 [SMILES: *C(=O)c1cnco1 ; *C(=O)c1cnc[nH]1 ; *C(=O)c1ccno1 ; *C(=O)c1ccon1]

In some embodiments, R^{a} is

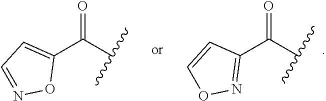 [SMILES: *C(=O)c1ccno1 ; *C(=O)c1ccon1]

In some embodiments, R^{a} is

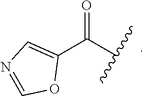 [SMILES: *C(=O)c1cnco1]

In some embodiments, R^{a} is

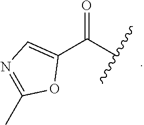 [SMILES: *C(=O)c1cnc(C)o1]

In some embodiments, R^{a} is

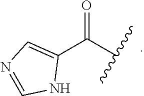 [SMILES: *C(=O)c1cnc[nH]1]

In some embodiments, R^{a} is

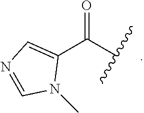 [SMILES: *C(=O)c1cncn1C]

In some embodiments, R^{a} is

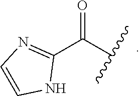 [SMILES: *C(=O)c1ncc[nH]1]

In some embodiments, R^{a} is

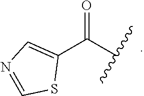 [SMILES: *C(=O)c1cncs1]

In some embodiments, R^{a} is

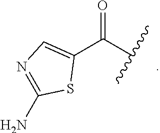 [SMILES: *C(=O)c1cnc(N)s1]

In some embodiments, R^{a} is

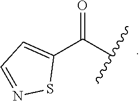 [SMILES: *C(=O)c1ccns1]

In some embodiments, R^{a} is

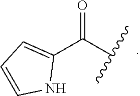 [SMILES: *C(=O)c1ccc[nH]1]

In some embodiments, R^{a} is

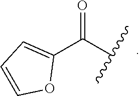 [SMILES: *C(=O)c1ccco1]

In some embodiments, R^{a} is

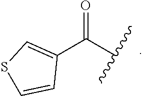 [SMILES: *C(=O)c1ccsc1]

In some embodiments, R^{a} is

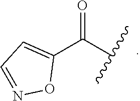 [SMILES: *C(=O)c1ccno1]

In some embodiments, R^{a} is

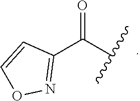 [SMILES: *C(=O)c1ccon1]

In some embodiments, R^{a} is

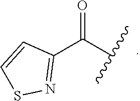 [SMILES: *C(=O)c1ccsn1]

In some embodiments, R^{a} is

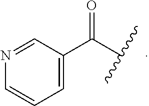 [SMILES: *C(=O)c1cccnc1]

In some embodiments, R^{a} is

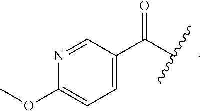 [SMILES: *C(=O)c1ccc(OC)nc1]

In some embodiments, R^{a} is

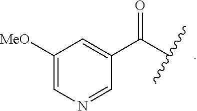 [SMILES: *C(=O)c1cncc(OC)c1]

In some embodiments, R^{a} is

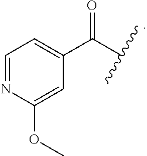 [SMILES: *C(=O)c1ccnc(OC)c1]

In some embodiments, R^{a} is

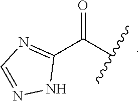 [SMILES: *C(=O)c1ncn[nH]1]

In some embodiments, R^{a} is

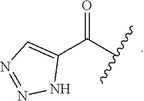 [SMILES: *C(=O)c1cnn[nH]1]

In some embodiments, R^{a} is

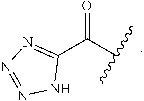 [SMILES: *C(=O)c1nnn[nH]1]

In some embodiments, R^{a} is

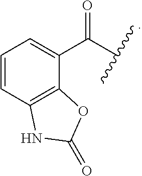 [SMILES: *C(=O)c1cccc2[nH]c(=O)oc12]

In some embodiments, R^{a} is

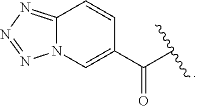 [SMILES: *C(=O)c1ccc2nnnn2c1]

In some embodiments, R^{a} is

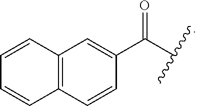 [SMILES: *C(=O)c1ccc2ccccc2c1]

In some embodiments, R^{a} is

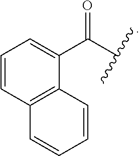 [SMILES: *C(=O)c1cccc2ccccc12]

In some embodiments, R^{a} is

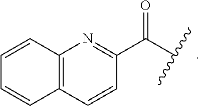 [SMILES: *C(=O)c1ccc2ccccc2n1]

In some embodiments, R^{a} is

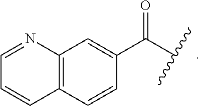 [SMILES: *C(=O)c1ccc2cccnc2c1]

In some embodiments, R^{a} is

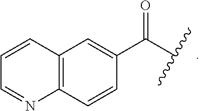 [SMILES: *C(=O)c1ccc2ncccc2c1]

In some embodiments, R^{a} is

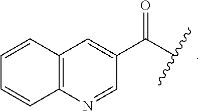 [SMILES: *C(=O)c1cnc2ccccc2c1]

In some embodiments, R^{a} is

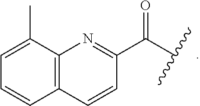 [SMILES: *C(=O)c1ccc2cccc(C)c2n1]

In some embodiments, R^{a} is

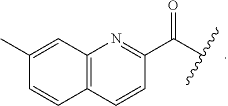 [SMILES: *C(=O)c1ccc2ccc(C)cc2n1]

In some embodiments, R^{a} is

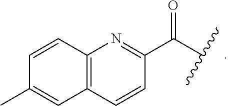 [SMILES: *C(=O)c1ccc2cc(C)ccc2n1]

In some embodiments, R^{a} is

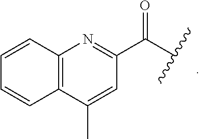 [SMILES: *C(=O)c1cc(C)c2ccccc2n1]

In some embodiments, R^{a} is

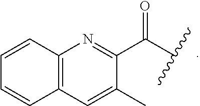 [SMILES: *C(=O)c1nc2ccccc2cc1C]

In some embodiments, R^{a} is

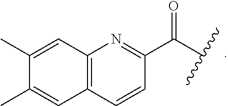 [SMILES: *C(=O)c1ccc2cc(C)c(C)cc2n1]

In some embodiments, R^{a} is

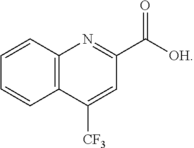 [SMILES: O=C(O)c1cc(C(F)(F)F)c2ccccc2n1]

In some embodiments, R^{a} is

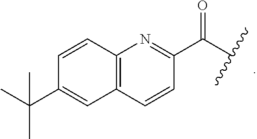 [SMILES: *C(=O)c1ccc2cc(C(C)(C)C)ccc2n1]

In some embodiments, R^{a} is

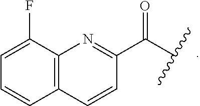 [SMILES: *C(=O)c1ccc2cccc(F)c2n1]

In some embodiments, R^{a} is

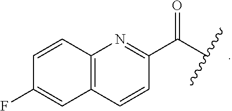 [SMILES: *C(=O)c1ccc2cc(F)ccc2n1]

In some embodiments, R^{a} is

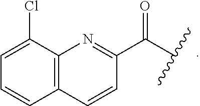 [SMILES: *C(=O)c1ccc2cccc(Cl)c2n1]

In some embodiments, R^{a} is

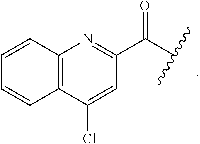 [SMILES: *C(=O)c1cc(Cl)c2ccccc2n1]

In some embodiments, R^{a} is

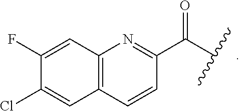 [SMILES: *C(=O)c1ccc2cc(Cl)c(F)cc2n1]

In some embodiments, R^{a} is

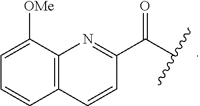 [SMILES: *C(=O)c1ccc2cccc(OC)c2n1]

In some embodiments, R^{a} is

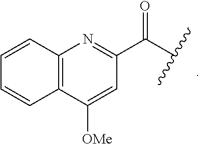 [SMILES: *C(=O)c1cc(OC)c2ccccc2n1]

In some embodiments, R^{a} is

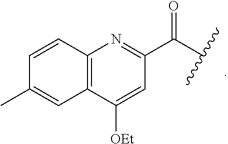 [SMILES: *C(=O)c1cc(OCC)c2cc(C)ccc2n1]

In some embodiments, R^{a} is

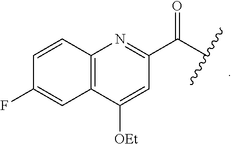 [SMILES: *C(=O)c1cc(OCC)c2cc(F)ccc2n1]

In some embodiments, R^{a} is

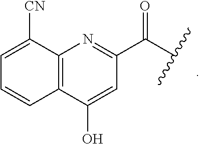 [SMILES: *C(=O)c1cc(O)c2cccc(C#N)c2n1]

In some embodiments, R^{a} is

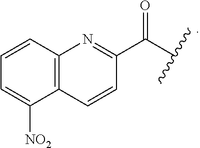 [SMILES: *C(=O)c1ccc2c([N+](=O)[O-])cccc2n1]

In some embodiments, R^{a} is

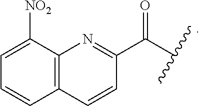 [SMILES: *C(=O)c1ccc2cccc([N+](=O)[O-])c2n1]

In some embodiments, R^{a} is

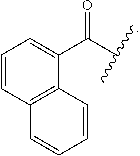 [SMILES: *C(=O)c1cccc2ccccc12]

In some embodiments, R^{a} is

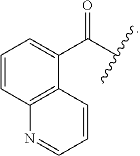 [SMILES: *C(=O)c1cccc2ncccc12]

In some embodiments, R^{a} is

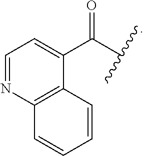 [SMILES: *C(=O)c1ccnc2ccccc12]

In some embodiments, R^{a} is

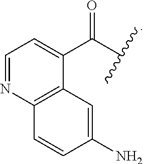 [SMILES: *C(=O)c1ccnc2ccc(N)cc12]

In some embodiments, R^{a} is

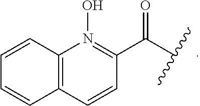

In some embodiments, R^{a} is

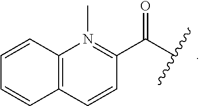

In some embodiments, R^{a} is

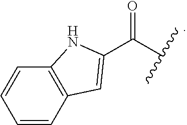 [SMILES: *C(=O)c1cc2ccccc2[nH]1]

In some embodiments, R^{a} is

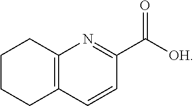 [SMILES: O=C(O)c1ccc2c(n1)CCCC2]

In some embodiments, R^{a} is

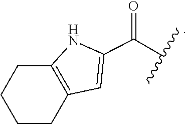 [SMILES: *C(=O)c1cc2c([nH]1)CCCC2]

In some embodiments, R^{a} is

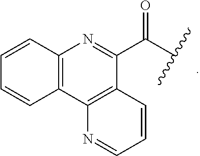 [SMILES: *C(=O)c1nc2ccccc2c2ncccc12]

In some embodiments, R^{a} is

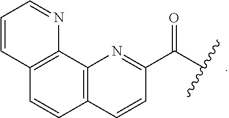 [SMILES: *C(=O)c1ccc2ccc3cccnc3c2n1]

In some embodiments, R^{a} is

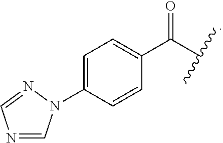 [SMILES: *C(=O)c1ccc(-n2cncn2)cc1]

In some embodiments, R^{a} is

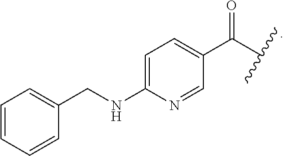 [SMILES: *C(=O)c1ccc(NCc2ccccc2)nc1]

In some embodiments, R^{a} is

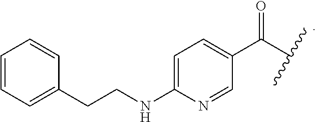 [SMILES: *C(=O)c1ccc(NCCc2ccccc2)nc1]

In some embodiments, R^{a} is

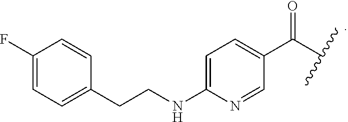 [SMILES: *C(=O)c1ccc(NCCc2ccc(F)cc2)nc1]

In some embodiments, R^{a} is

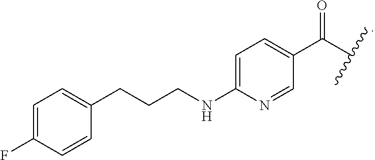 [SMILES: *C(=O)c1ccc(NCCCc2ccc(F)cc2)nc1]

In some embodiments, R^{a} is

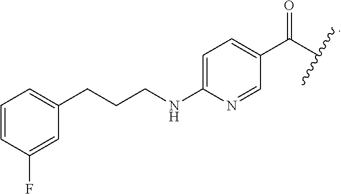 [SMILES: *C(=O)c1ccc(NCCCc2cccc(F)c2)nc1]

In some embodiments, R^{a} is

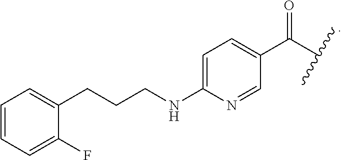 [SMILES: *C(=O)c1ccc(NCCCc2ccccc2F)nc1]

In some embodiments, R^{a} is

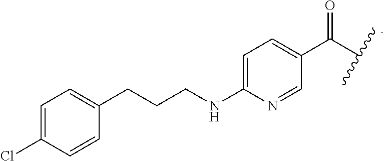 [SMILES: *C(=O)c1ccc(NCCCc2ccc(Cl)cc2)nc1]

In some embodiments, R^{a} is

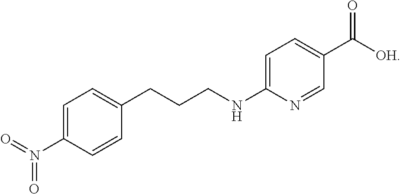 [SMILES: O=C(O)c1ccc(NCCCc2ccc([N+](=O)[O-])cc2)nc1]

In some embodiments, R^{a} is

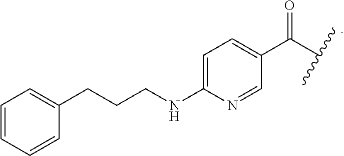 [SMILES: *C(=O)c1ccc(NCCCc2ccccc2)nc1]

In some embodiments, R^{a} is

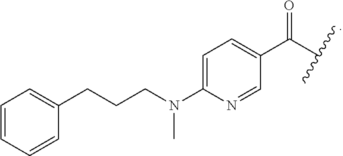 [SMILES: *C(=O)c1ccc(N(C)CCCc2ccccc2)nc1]

In some embodiments, R^{a} is

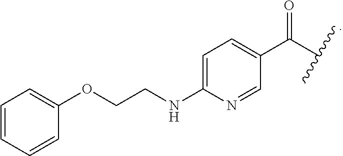 [SMILES: *C(=O)c1ccc(NCCOc2ccccc2)nc1]

In some embodiments, R^{a} is

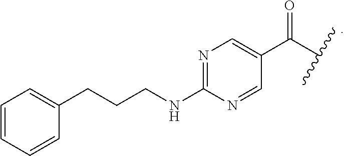 [SMILES: *C(=O)c1cnc(NCCCc2ccccc2)nc1]

In some embodiments, R^{a} is

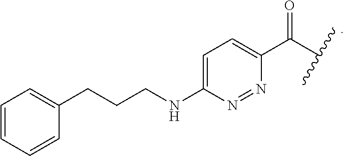 [SMILES: *C(=O)c1ccc(NCCCc2ccccc2)nn1]

In some embodiments, R^{a} is

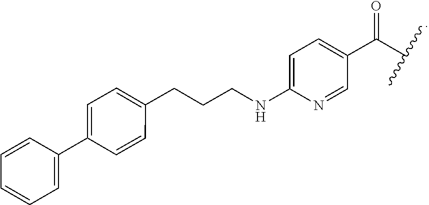 [SMILES: *C(=O)c1ccc(NCCCc2ccc(-c3ccccc3)cc2)nc1]

In some embodiments, R^{a} is

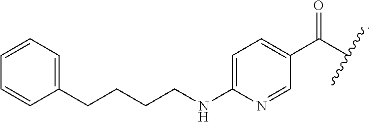 [SMILES: *C(=O)c1ccc(NCCCCc2ccccc2)nc1]

In some embodiments, R^{a} is

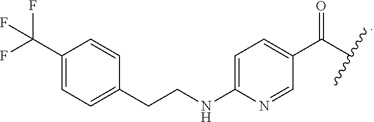 [SMILES: *C(=O)c1ccc(NCCc2ccc(C(F)(F)F)cc2)nc1]

In some embodiments, R^{a} is

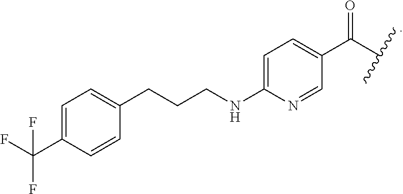 [SMILES: *C(=O)c1ccc(NCCCc2ccc(C(F)(F)F)cc2)nc1]

In some embodiments, R^{a} is

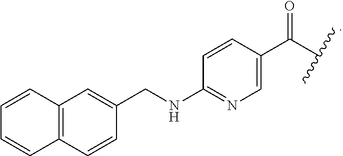 [SMILES: *C(=O)c1ccc(NCc2ccc3ccccc3c2)nc1]

In some embodiments, R^{a} is

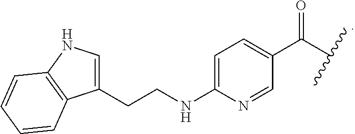 [SMILES: *C(=O)c1ccc(NCCc2c[nH]c3ccccc23)nc1]

In some embodiments, R^{a} is

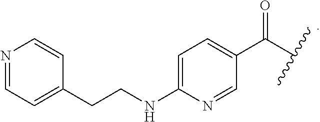 [SMILES: *C(=O)c1ccc(NCCc2ccncc2)nc1]

In some embodiments, R^{a} is

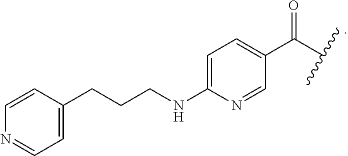 [SMILES: *C(=O)c1ccc(NCCCc2ccncc2)nc1]

In some embodiments, R^{a} is

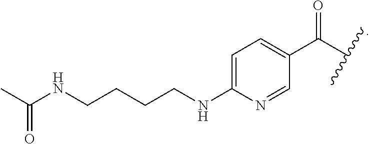 [SMILES: *C(=O)c1ccc(NCCCCNC(C)=O)nc1]

In some embodiments, R^{a} is

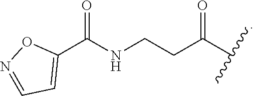 [SMILES: *C(=O)CCNC(=O)c1ccno1]

In some embodiments

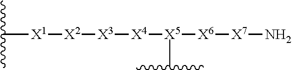

is

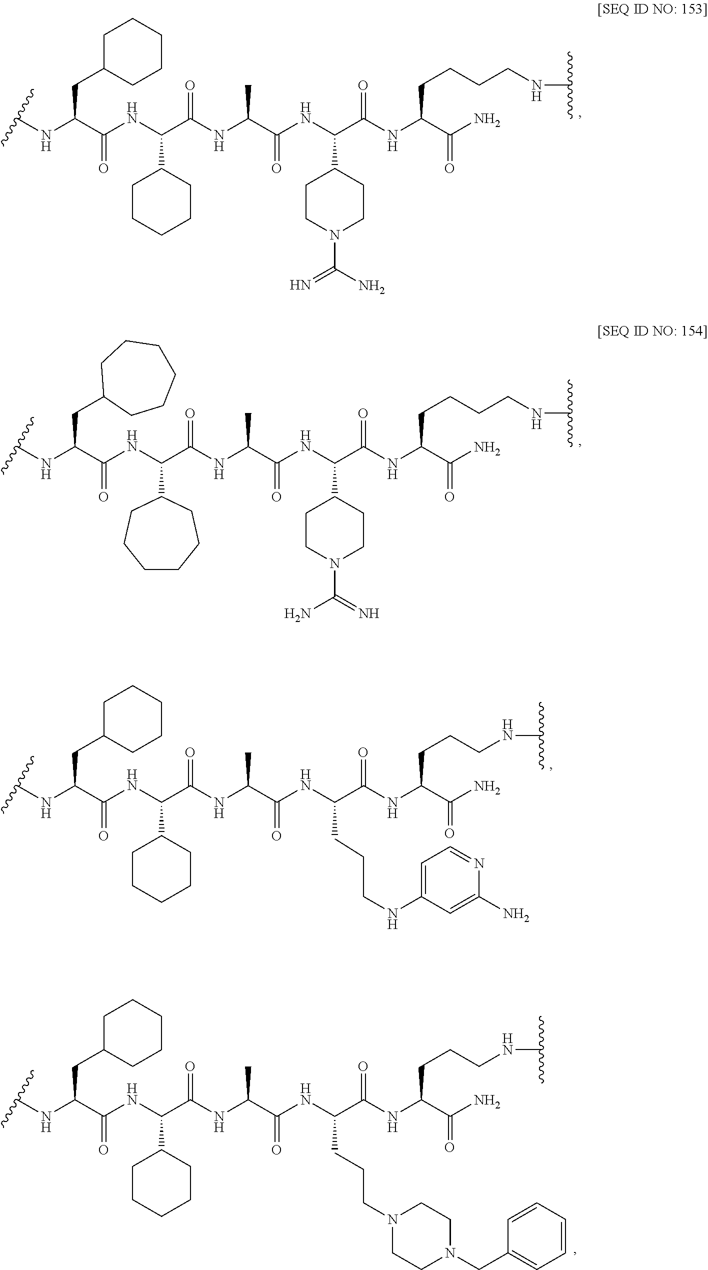 [SMILES: *NCCCC[C@H](NC(=O)[C@@H](NC(=O)[C@H](C)NC(=O)[C@@H](NC(=O)[C@H](CC1CCCCC1)N*)C1CCCCC1)C1CCN(C(=N)N)CC1)C(N)=O ; *NCCCC[C@H](NC(=O)[C@@H](NC(=O)[C@H](C)NC(=O)[C@@H](NC(=O)[C@H](CC1CCCCCC1)N*)C1CCCCCC1)C1CCN(C(=N)N)CC1)C(N)=O ; *NCCC[C@H](NC(=O)[C@H](CCCNc1ccnc(N)c1)NC(=O)[C@H](C)NC(=O)[C@@H](NC(=O)[C@H](CC1CCCCC1)N*)C1CCCCC1)C(N)=O ; *NCCC[C@H](NC(=O)[C@H](CCCN1CCN(Cc2ccccc2)CC1)NC(=O)[C@H](C)NC(=O)[C@@H](NC(=O)[C@H](CC1CCCCC1)N*)C1CCCCC1)C(N)=O]

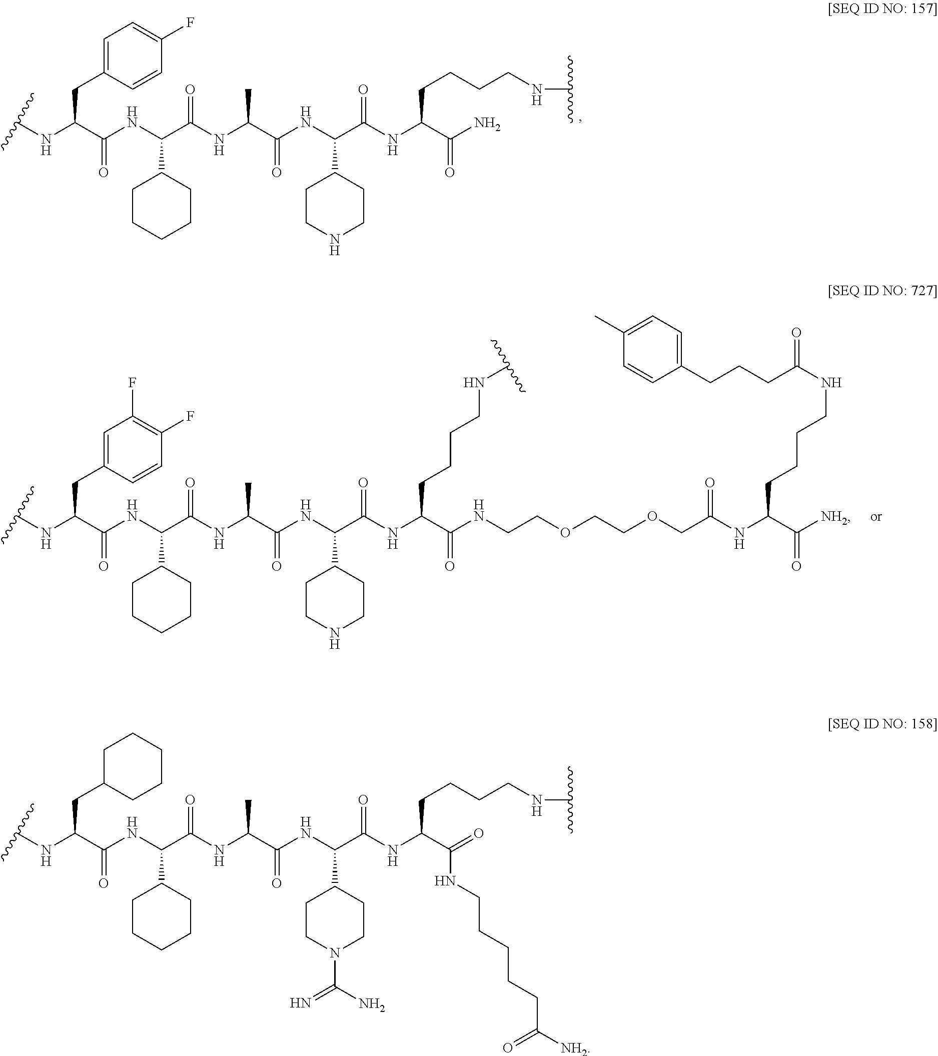 [SMILES: *NCCCC[C@H](NC(=O)[C@@H](NC(=O)[C@H](C)NC(=O)[C@@H](NC(=O)[C@H](Cc1ccc(F)cc1)N*)C1CCCCC1)C1CCNCC1)C(N)=O ; *NCCCC[C@H](NC(=O)[C@@H](NC(=O)[C@H](C)NC(=O)[C@@H](NC(=O)[C@H](Cc1ccc(F)c(F)c1)N*)C1CCCCC1)C1CCNCC1)C(=O)NCCOCCOCC(=O)N[C@@H](CCCCNC(=O)CCCc1ccc(C)cc1)C(N)=O ; *NCCCC[C@H](NC(=O)[C@@H](NC(=O)[C@H](C)NC(=O)[C@@H](NC(=O)[C@H](CC1CCCCC1)N*)C1CCCCC1)C1CCN(C(=N)N)CC1)C(=O)NCCCCCC(N)=O]

In some embodiments,

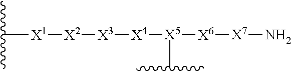

is:

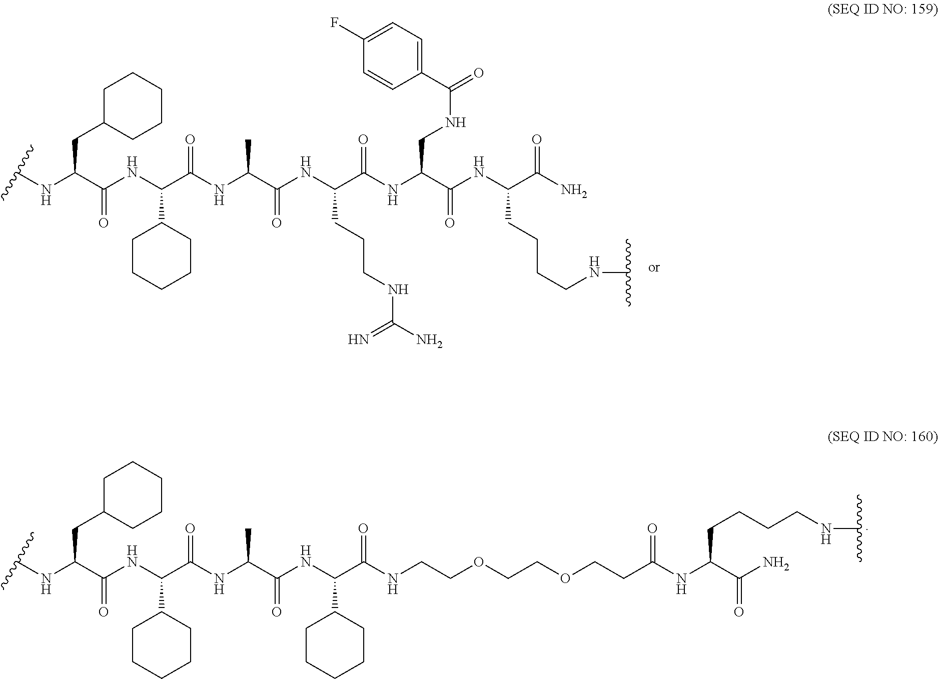 [SMILES: *NCCCC[C@H](NC(=O)[C@H](CNC(=O)c1ccc(F)cc1)NC(=O)[C@H](CCCNC(=N)N)NC(=O)[C@H](C)NC(=O)[C@@H](NC(=O)[C@H](CC1CCCCC1)N*)C1CCCCC1)C(N)=O ; *NCCCC[C@H](NC(=O)CCOCCOCCNC(=O)[C@@H](NC(=O)[C@H](C)NC(=O)[C@@H](NC(=O)[C@H](CC1CCCCC1)N*)C1CCCCC1)C1CCCCC1)C(N)=O]

In some embodiments, the compound of Formula (I) is a compound of Formula (II) or Formula (III):

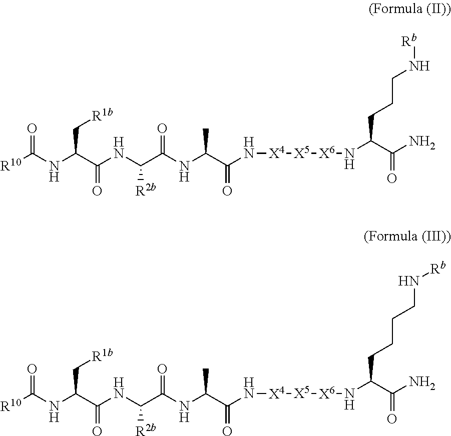

R¹⁰ is a monocyclic heteroaryl selected from the group consisting of imidazolyl, pyrazolyl, triazolyl, furyl, thienyl, isoxazolyl, thiazolyl, oxazolyl, isothiazolyl, and pyrrolyl; and wherein R¹⁰ is optionally substituted with 1 to 3 substituents that are each independently selected from the group consisting of halo and C₁-C₄ alkyl;
R^{1b} is cyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, or perfluorophenyl;
R^{2b} is cyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, or perfluorophenyl;
X⁴ is (S)-arginine (Arg), (S)-2-(piperidin-4-yl)-glycine (Gly(piperidin-4-yl))

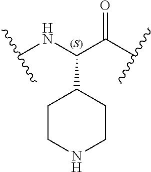

(S)-2-(1-carbamimidoylpiperidin-4-yl)-glycine (Gly(1-carbamimidoylpiperidin-4-yl))

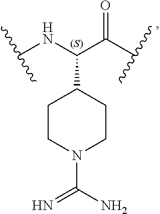

### (S)-(pyridin-4-yl)alanine (3-Pal),

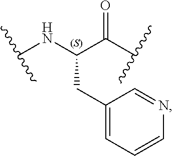

(S)-(pyridin-4-yl)alanine (4-Pal)

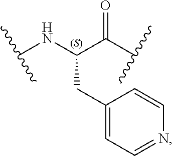

(S)-3-(6-amninopyridin-3-yl)alanine ((3-Pal(6-NH₂))

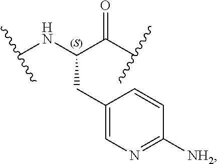

(S)-2-((2-aminopyridin-4-yl)-λ²-azaneyl)ethyl alanine

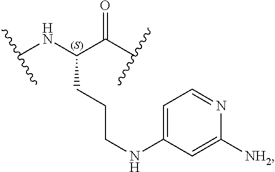

(S)-2-(piperidin-4-yl)ethyl alanine

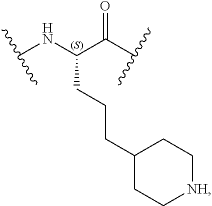

(S)-3-((piperazin-1-yl)propyl)-glycine

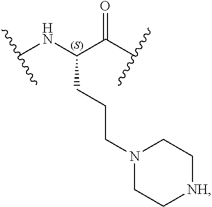

(S)-3-((4-methylpiperazin-1-yl)propyl)-glycine

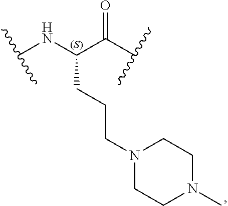

(S)-3-(4-benzylpiperidin-1-yl)propylglycine

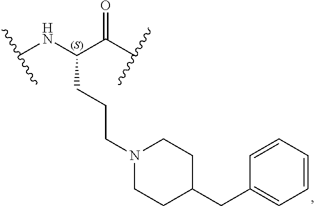

(S)-3-(4-benzylpiperidin-1-yl)propylglycine

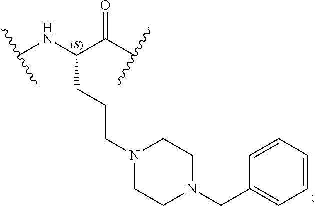

X⁵ is absent, or (4-fluorobenzamido)methylglycine

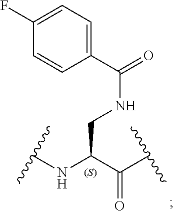

X⁶ is absent, or -L⁶-;
L⁶ is —NR¹⁵-L¹³-L¹⁴-C(═O)—;
L¹³ is —C₁-C₆ alkylene, or —(CH₂CH₂O)_{y}—;
L¹⁴ is absent, —C₁-C₆ alkylene;
R¹⁵ is H or —C₁-C₄ alkyl;
y is 1, 2, 3, 4, 5, or 6; and
R^{b} is

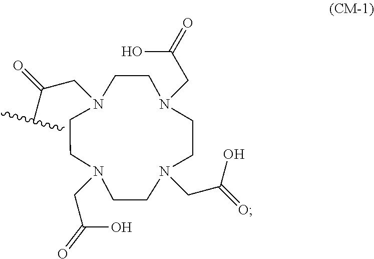 [SMILES: *C(=O)CN1CCN(CC(=O)O)CCN(CC(=O)O)CCN(CC(=O)O)CC1]

or a radionuclide complex thereof.

In some embodiments, the compound of Formula. (I) has one of the following chemical structures:

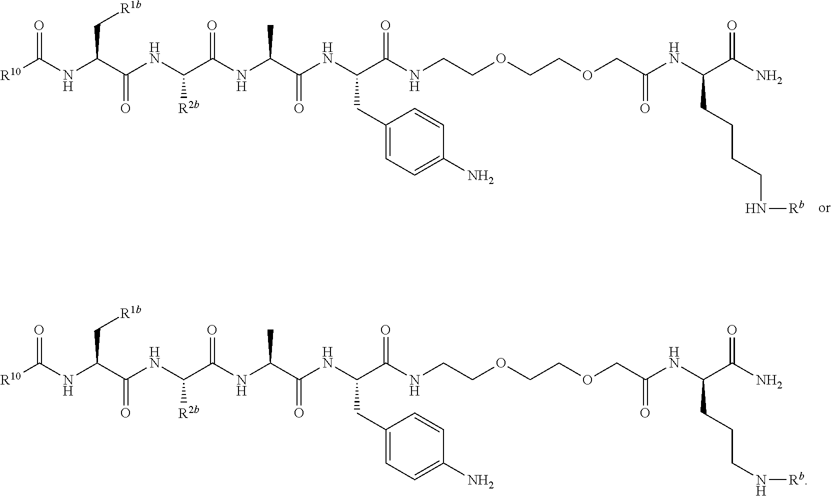

In some embodiments, R¹¹ is a monocyclic heteroaryl selected from the group consisting of imidazolyl, pyrazolyl, triazolyl, furyl, thienyl, isoxazolyl, thiazolyl, oxazolyl, isothiazolyl, and pyrrolyl; and wherein R¹⁰ is optionally substituted with 1 to 3 substituents that are each independently selected from the group consisting of halo and C₁-C₄ alkyl; R^{1b} is cyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, or perfluorophenyl; R^{2b} is cyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, or perfluorophenyl; and R^{b} is

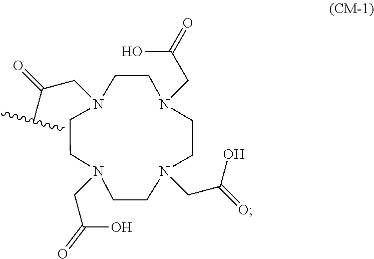 [SMILES: *C(=O)CN1CCN(CC(=O)O)CCN(CC(=O)O)CCN(CC(=O)O)CC1]

or a radionuclide complex thereof.

In some embodiments, the compound of Formula (I) is a compound of Formula (IV) or Formula (V):

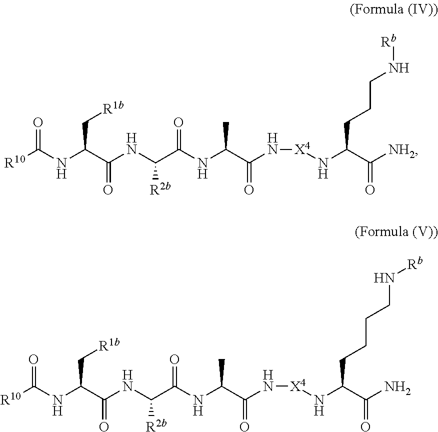

R¹⁰ is a monocyclic heteroaryl selected from the group consisting of imidazolyl, pyrazolyl, triazolyl, furyl, thienyl, isoxazolyl, thiazolyl, oxazolyl, isothiazolyl, and pyrrolyl; and wherein R¹⁰ is optionally substituted with 1 to 3 substituents that are each independently selected from the group consisting of halo and C₁-C₄ alkyl;
R^{1b} is cyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, or perfluorophenyl;
R^{2b} is cyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, or perfluorophenyl;
X⁴ is (S)-arginine (Arg), (S)-2-(piperidin-4-yl)-glycine (Gly(piperidin-4-yl))

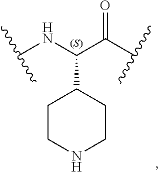

(S)-2-(1-carbamimidoylpiperidin-4-yl)-glycine (Gly(1-carbamimidoylpiperidin-4-yl))

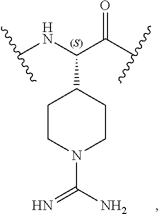

### (S)-(pyridin-4-yl)alanine (3-Pal),

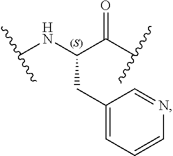

(S)-(pyridin-4-yl)alanine (4-Pal),

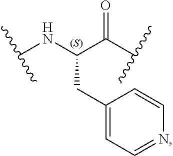

(S)-3-(6-aminopyridin-3-yl)alanine ((3-Pal(6—NH₂))

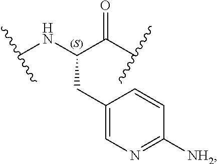

(S)-2-((2-aminopyridin-4-yl)-λ²-azaneyl)ethyl alanine

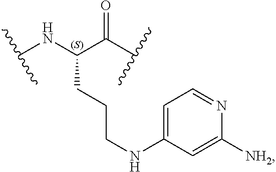

(S)-2-(piperidin-4-yl)ethyl alanine

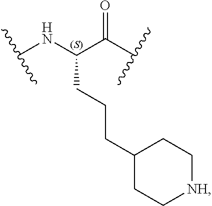

(S)-3-((piperazin-1-yl)propyl)-glycine

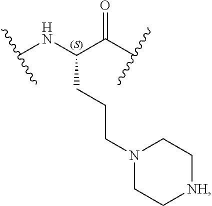

(S)-3-((4-methylpiperazin-1-yl)propyl)-glycine

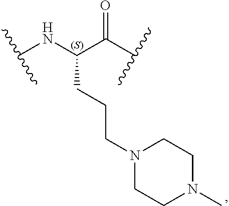

(S)-3-(4-benzylpiperidin-1-yl)propylglycine,

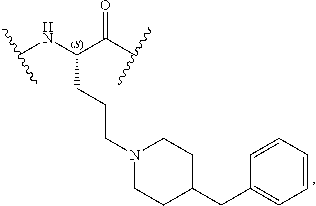

(S)-3-((4-benzylpiperazin-1-yl)propyl)-glycine

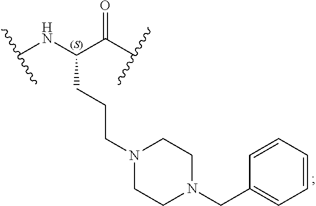

and
R^{b} is

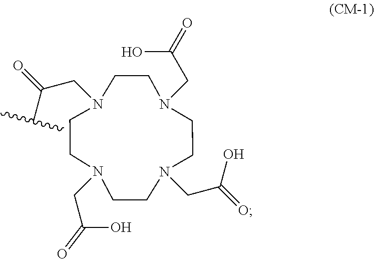 [SMILES: *C(=O)CN1CCN(CC(=O)O)CCN(CC(=O)O)CCN(CC(=O)O)CC1]

or a radionuclide complex thereof.

In some embodiments, the compound of Formula (I) has one of the following chemical structures:

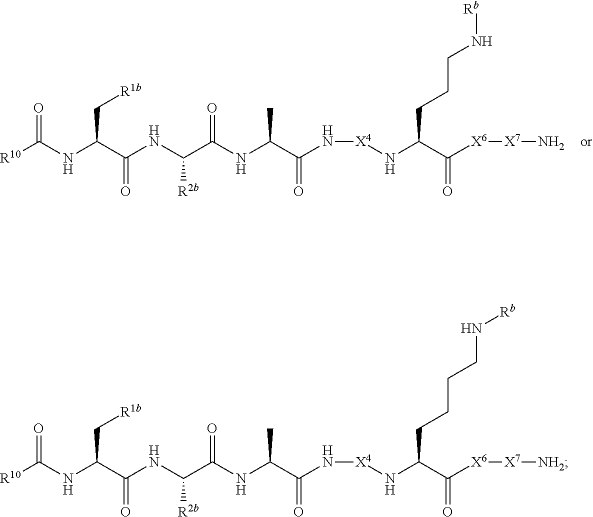

wherein:
R¹⁰ is a monocyclic heteroaryl selected from the group consisting of imidazolyl, pyrazolyl, triazolyl, furyl, thienyl, isoxazolyl, thiazolyl, oxazolyl, isothiazolyl, and pyrrolyl; and wherein R¹⁰ is optionally substituted with 1 to 3 substituents that are each independently selected from the group consisting of halo and C₁-C₄ alkyl;
R^{1b} is cyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, or perfluorophenyl;
R^{2b} is cyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, or perfluorophenyl;
X⁴ is (S)-arginine (Arg), (S)-2-(piperidin-4-yl)-glycine (Gly(piperidin-4-yl))

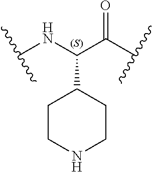

(S)-2-(1-carbamimidoylpiperidin-4-yl)-glycine (Gly(1-carbamimidoylpiperidin-4-yl))

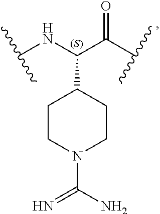

### (S)-(pyridin-4-yl)alanine (3-Pal)

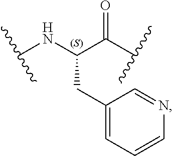

(S)-(pyridin-4-yl)alanine (4-Pal)

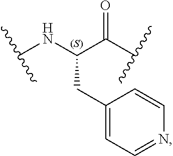

(S)-3-(6-aminopyridin-3-yl)alanine ((3-Pal(6-NH₂))

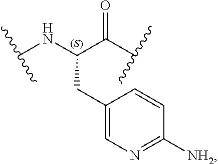

(S)-2-((2-aminopyridin-4-yl)-λ²-azaneyl)ethyl alanine

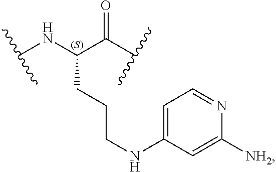

(S)-2-(piperidin-4-yl)ethyl alanine

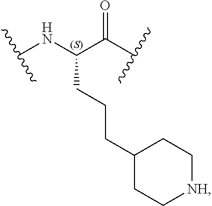

(S)-3-((piperazin-1-yl)propyl)-glycine

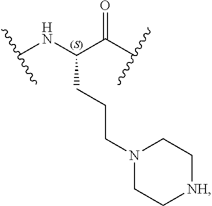

(S)-3-((4-methylpiperazin-1-yl)propyl)-glycine

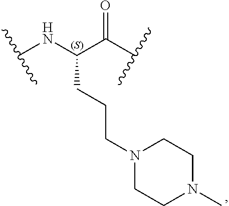

(S)-3-(4-benzylpiperidin-1-yl)propylglycine

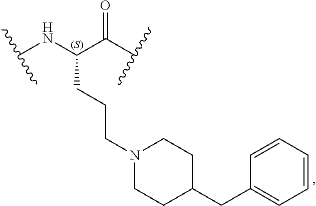

(S)-3-((4-benzylpiperazin-1-yl)propyl)-glycine

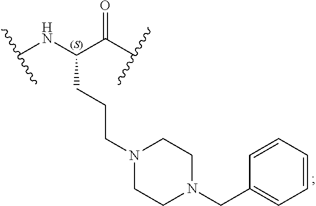

X⁶ is

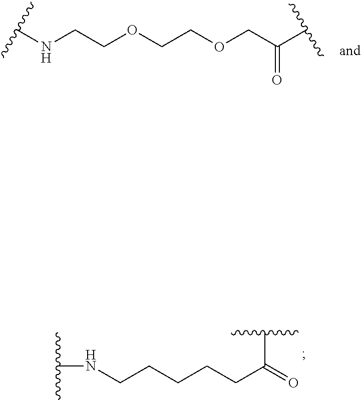 [SMILES: *NCCOCCOCC(*)=O ; *NCCCCCC(*)=O]

X⁷ is absent, lysine (Lys), ornithine (Orn), or

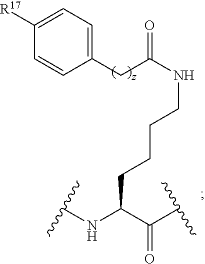

R¹⁷ is —CH₃ or —I;
z is 1, 2, or 3; and
R^{b} is

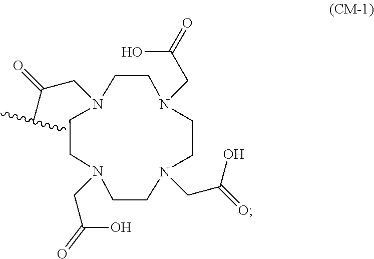 [SMILES: *C(=O)CN1CCN(CC(=O)O)CCN(CC(=O)O)CCN(CC(=O)O)CC1]

or a radionuclide complex thereof. In some embodiments, the compound of Formula (I) has one of the following chemical structures:

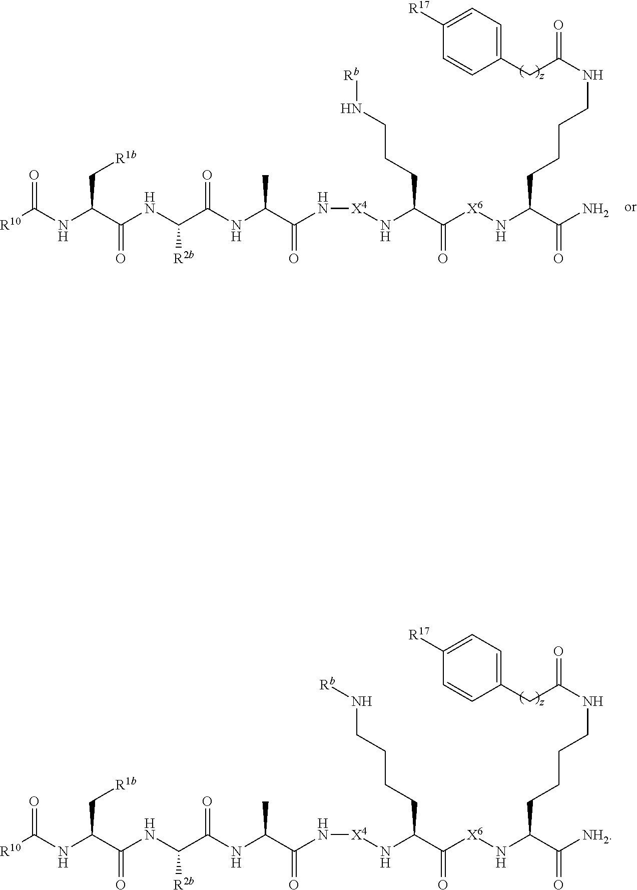

### Radionuclide Complexes

Radiopharmaceuticals have increasingly become very useful tools for physicians to diagnose, stage, treat, and monitor the progression of several diseases, especially cancer. The primary difference between radiopharmaceuticals and other pharmaceutical drugs is that radiopharmaceuticals contain a radionuclide. The nuclear decay properties of the radionuclide determine whether a radiopharmaceutical will be used clinically as a diagnostic agent or as a therapeutic agent. Diagnostic radiopharmaceuticals require radionuclides that emit either gamma (γ) rays or positrons (β+), which subsequently annihilate with nearby electrons to produce two 511 keV annihilation photons emitted approximately 180° away from each other. Gamma ray-emitting radionuclides (e. g. ^{99m}Tc, ¹¹¹In, ²⁰¹Tl, etc.) are useful for single photon emission computed tomography (SPECT), while positron-emitting radionuclides (e. g. ¹⁸F, ⁸⁹Zr, ⁶⁸Ga, etc.) are useful for positron emission tomography (PET).

In contrast, therapeutic radiopharmaceuticals require radionuclides that emit particulate radiation, such as alpha (α) particles, beta (β−) particles, or Auger electrons. These particles, which strongly interact with target tissues (e. g. cancerous tumor) and lead to extensive localized ionization, can damage chemical bonds in DNA molecules and potentially induce cytotoxicity.

For most nuclear medicine applications, it is desired that a diagnostic radiopharmaceutical is paired with a therapeutic radiopharmaceutical. This concept is commonly known as “theranostics”. As a first step in the theranostic concept, a target molecule labeled with a diagnostic radionuclide is used for quantitative imaging of a tumor imaging biomarker, either by positron emission tomography (PET) or single photon emission computed tomography (SPECT). When it is demonstrated that, with this targeted molecule, a tumoricidal radiation absorbed dose can be delivered to tumor and metastases, as a second step, via administration of the same or a similar target molecule labeled with a therapeutic radionuclide.

In some embodiments, the chemical and pharmacokinetic behaviors of both the diagnostic and therapeutic radiopharmaceuticals match. In some embodiments, the diagnostic and therapeutic radionuclides are a chemically identical radioisotope pair (also known as a “matched pair”). One examples of a matched pair for theranostic radiopharmaceutical applications is the ¹²³I/¹³¹I pair, where ¹²³I-labeled compounds are used for diagnosis, while ¹³¹I-labeled compounds are used for therapy. Other theranostic matched pairs include ⁴⁴Sc/⁴⁷Sc, ⁶⁴Ci/⁶⁷Cu, ⁷²As/⁷⁷As, ⁸⁶Y/⁹⁰Y, and ²⁰³Pb/²¹²Pb, among others. Alternatively, radionuclide pairs from different elements can be utilized for theranostic radiopharmaceutical development when their chemistry is very similar (e. g. ^{99m}Tc/^{186/188}Re) and there is no significant difference in the pharmacokinetic behavior between the diagnostic and therapeutic analogues. Another example is the ⁶⁸Ga/¹⁷⁷Lu pair, where ⁶⁸Ga is used for diagnosis and ¹⁷⁷Lu is used for therapy. For example, gastroenteropancreatic endocrine tumors express high amounts of sst2 receptor that can be targeted with somatostatin receptor scintigraphy for diagnostic purposes with a ⁶⁸Ga sst2 ligand conjugate ([⁶⁸Ga]Ga-DOTA-TATE (NETSPOT™) or [⁶⁸Ga]Ga-DOTA-TOC (DOTA-(D-Phe1,Tyr3)-octreotide, SomaKit TOC®)), followed by treatment with a ¹⁷⁷Lu sst2 ligand conjugate ([¹⁷⁷Lu]Lu-DOTA-TATE) for endoradiotherapy.

### Chelating Moieties Used to Generate Metal (Radionuclide) Complexes

The compounds described herein comprise at least one R^{b} group, wherein R^{b} is a chelating moiety capable of chelating a radionuclide (Z′), or radionuclide complex thereof. In some embodiments, any suitable group or atom(s) of the chelator are used to connect, via an optional linker, to the PAR2 targeting ligand.

In some embodiments, the chelator is capable of binding a radioactive atom. In some embodiments, the binding is direct, e.g., the chelator makes hydrogen bonds or electrostatic interactions with a radioactive atom. In some embodiments, the binding is indirect, e.g., the chelator binds to a molecule that comprises a radioactive atom. In some embodiments, the chelator is or comprises a macrocycle.

In some embodiments, the chelator comprises one or more amine groups. In some embodiments, the metal chelator comprises two or more amine groups. In some embodiments, the chelator comprises three or more amine groups. In some embodiments, the chelator comprises four or more amine groups. In some embodiments, the chelator includes 4 or more N atoms, 4 or more carboxylic acid groups, or a combination thereof. In some embodiments, the chelator does not comprise S. In some embodiments, the chelator comprises a ring. In some embodiments, the ring comprises an O and/or a N atom. In some embodiments, the chelator is a ring that includes 3 or more N atoms, 3 or more carboxylic acid groups, or a combination thereof. In some embodiments, the chelator is polydentate ligand, bidentate ligand, or monodentate ligand. Polydentate ligands range in the number of atoms used to bond to a metal atom or ion. EDTA, a hexadentate ligand, is an example of a polydentate ligand that has six donor atoms with electron pairs that can be used to bond to a central metal atom or ion. Bidentate ligands have two donor atoms which allow them to bind to a central metal atom or ion at two points. Ethylenediamine (en) and the oxalate ion (ox) are examples of bidentate ligands.

In some embodiments, a chelator described herein comprises a cyclic chelating agent or an acyclic chelating agent. In some embodiments, a chelator described herein comprises a cyclic chelating agent. In some embodiments, a chelator described herein comprises an acyclic chelating agent.

In some embodiments, a chelator described herein comprises cyclen, DO2A, DO3A, HP-DO3A, DO3A-Nprop, DO3AP, DO3AP^{PrA}, DO3AP^{ABn}, DO3AM^{nBu}, BT-DO3A, DOTA, PSC, DOTAGA, DOTA(GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE2A, TE2A, NOTA, NODAGA, NODA-MPAA, (3p-C-NETA), TACN, TACN-TM, NOTP, Sarcophagine (Sar), DiAmSar, SarAr, AmBaSar, cis-DO2A2P, trans-DO2A2P, DOTEP, p-NO₂-Bn-DOTA, BAT, DO3TMP-Monoamide, CHX-A″-DTPA, c-DEPA, PCTA, p-NO-Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa, CP256, THP, YM103, t-Bu-calix[4]arene-tetracarboxylic acid, CHX-A″-DTPA, H₆phospha, p-NH₂—Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), or SBTG₂DAP.

In some embodiments, a chelator described herein comprises DOTA, DOTAGA, DOTA(GA)₂, NOTA, NODAGA, TRITA, TETA, DOTA-MA, HP-DO3A, DOTMA, DOTA-pNB, DOTP, DOTMP, DOTEP, DOTMPE, F-DOTPME, DOTPP, DOTBzP, DOTA-monoamide, BAT, DO3TMP-Monoamide, and CHX-A″-DTPA.

In some embodiments, a chelator described herein comprises DTA, CyEDTA, EDTMP, DTPMP, DTPA, CyDTPA, Cy2DTPA, DTPA-MA, DTPA-BA, and BOPA.

In some embodiments, a chelator described herein comprises DOTA, DOTAGA, DOTA(GA)₂, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP.

In some embodiments, a chelator described herein comprises HP-DO3A, BT-DO3A, DO3A-Nprop, DO3AP, DO2A2P, DOA3P, DOTP, DOTPMB, DOTAMAE, DOTAMAP, DO3AM^{Bu}, DOTMA, TCE-DOTA, DEPA, PCTA, p-NO₂—Bn-PCTA, p-NO₂—Bn-DOTA, symPC2APA, symPCA2PA, asymPC2APA, asymPCA2PA, TRAP, AAZTA, DATA^{m}, THP, HEHA, HBED, or HBED-CC TFP.

In some embodiments, a chelator described herein comprises DOTA, NOTA, NODAGA, DOTAGA, HBED, HBED-CC TFP, H2DEPDPA, DFO-B, Deferiprone, CP256, YM103, TETA, CB-TE2A, TE2A, Sar, DiAmSar, TRAPH, TRAP-Pr, TRAP-OH, TRAP-Ph, NOPO, DEADPA, PCTA, EDTA, PEPA, HEHA, DTPA, EDTMP, AAZTA, DO3AP, DO3AP^{PrA}, DO3AP^{ABn}, or DOTAM.

In some embodiments, the chelator is or comprises DOTA, HBED-CC, DOTAGA, DOTA(GA)₂, NOTA, and DOTAM. In some embodiments, the chelator is or comprises NODAGA, NOTA, DOTAGA, DOTA(GA)₂, TRAP, NOPO, NCTA, DFO, DTPA, and HYNIC.

In some embodiments, the chelator comprises a macrocycle, e.g., a macrocycle comprising an O and/or a N atom, DOTA, HBED-CC, DOTAGA, DOTA(GA)₂, NOTA, DOTAM, one or more amines, one or more ethers, one or more carboxylic acids, EDTA, DTPA, TETA, DO3A, PCTA, or desferrioxamine.

In some embodiments, a metal chelator described herein comprises one of the following structures:

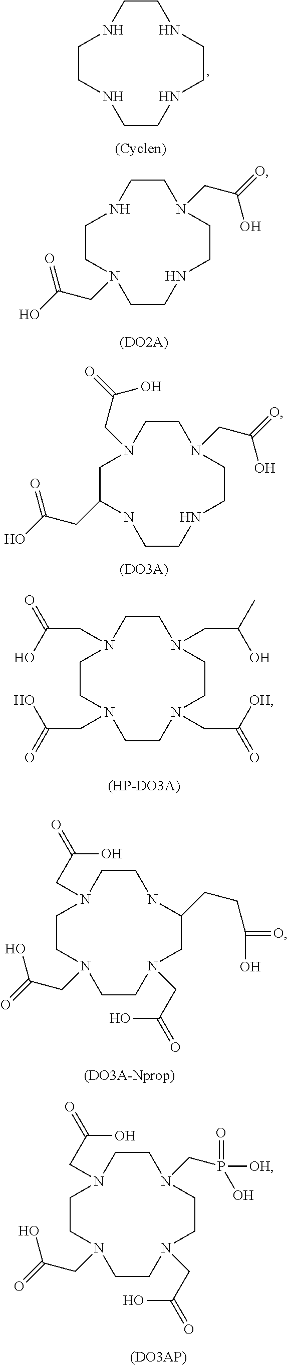

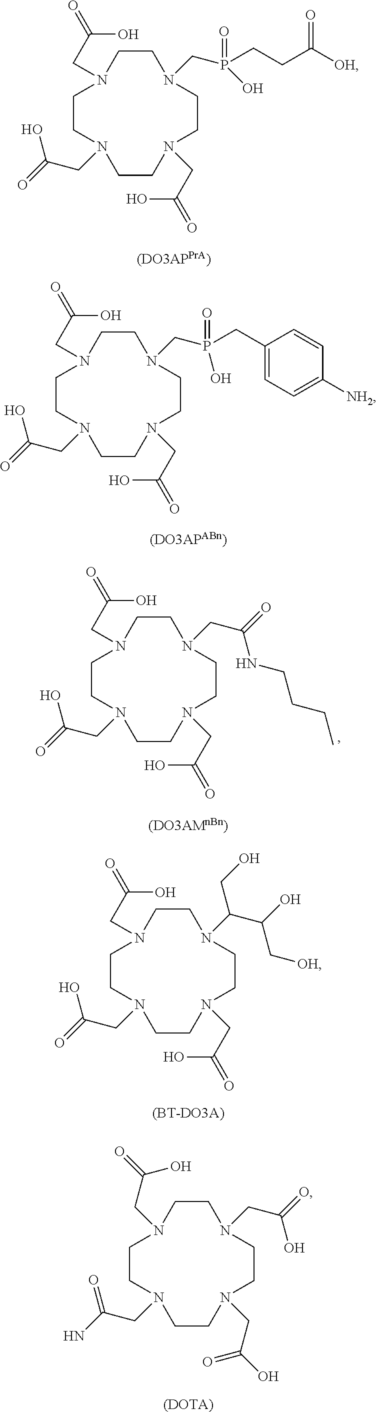

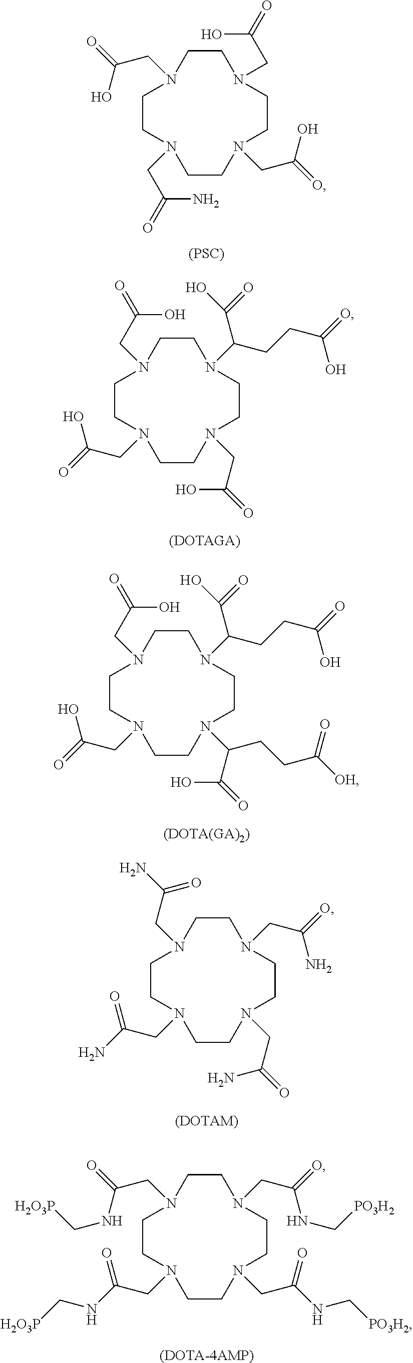

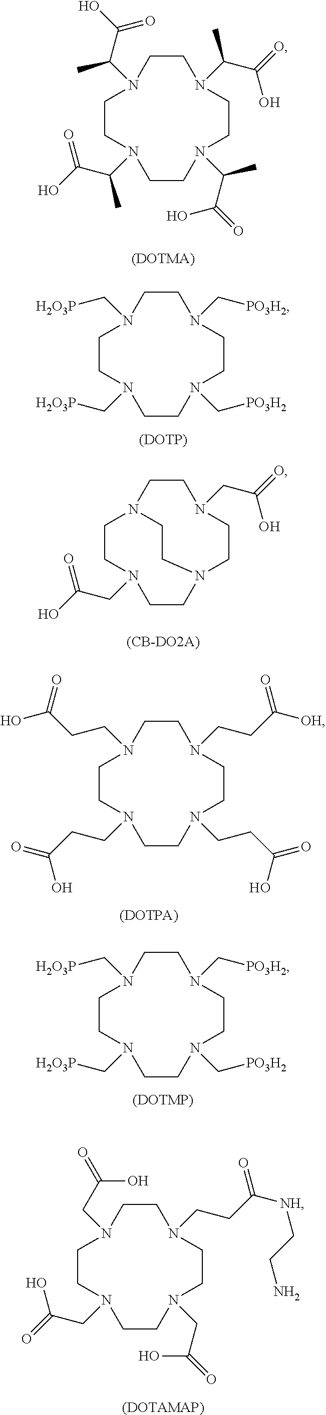

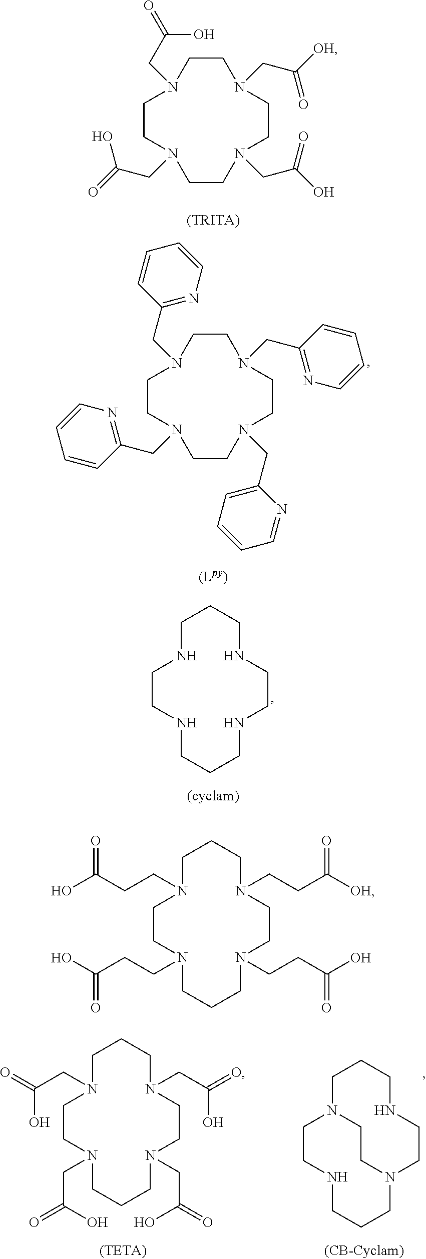

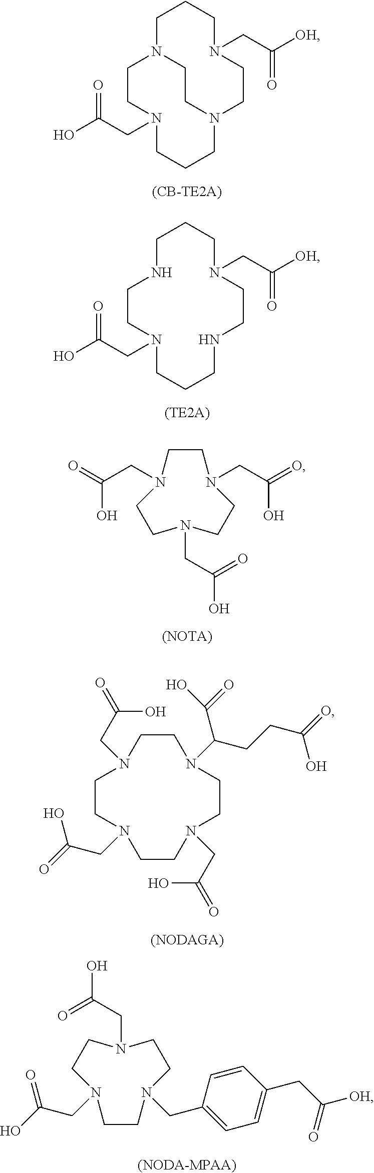

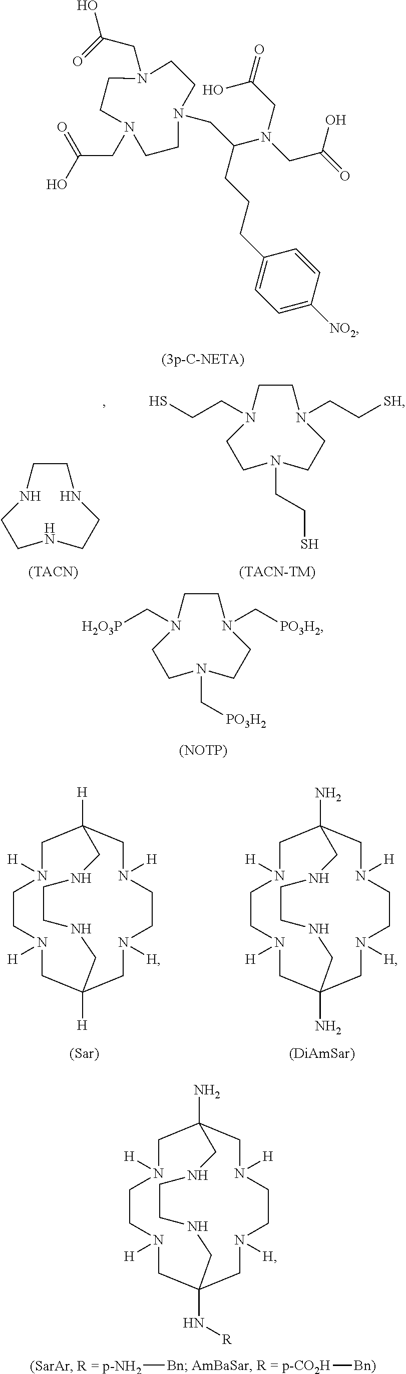

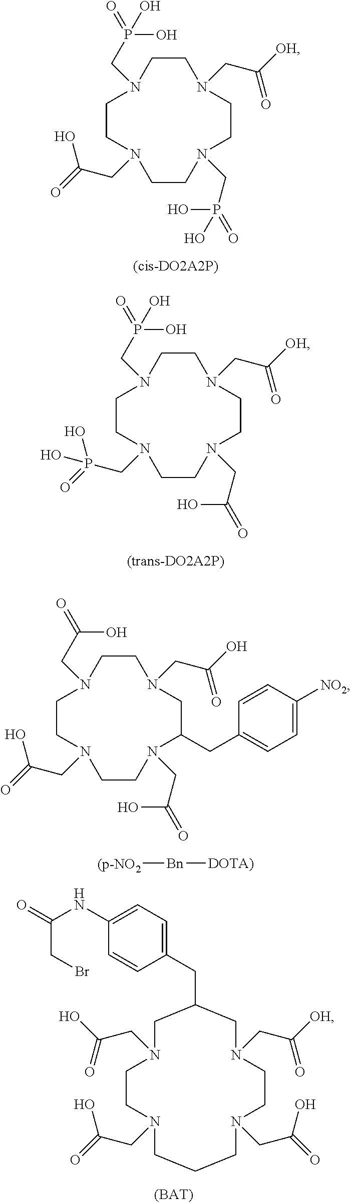

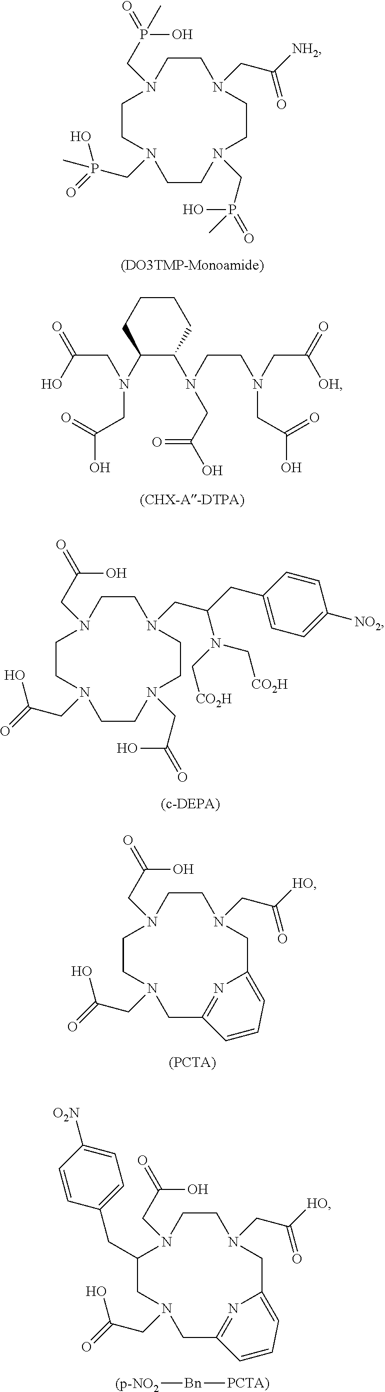

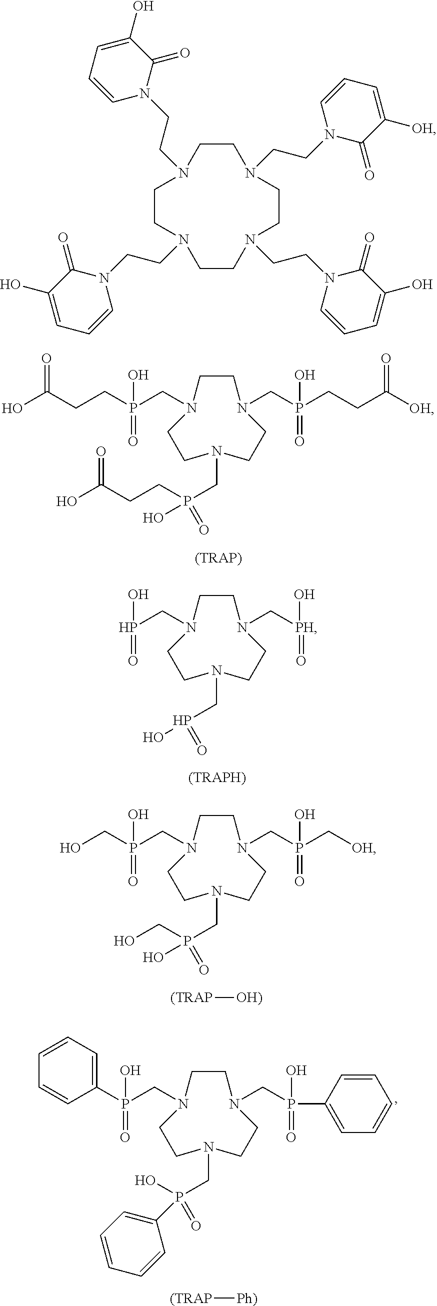

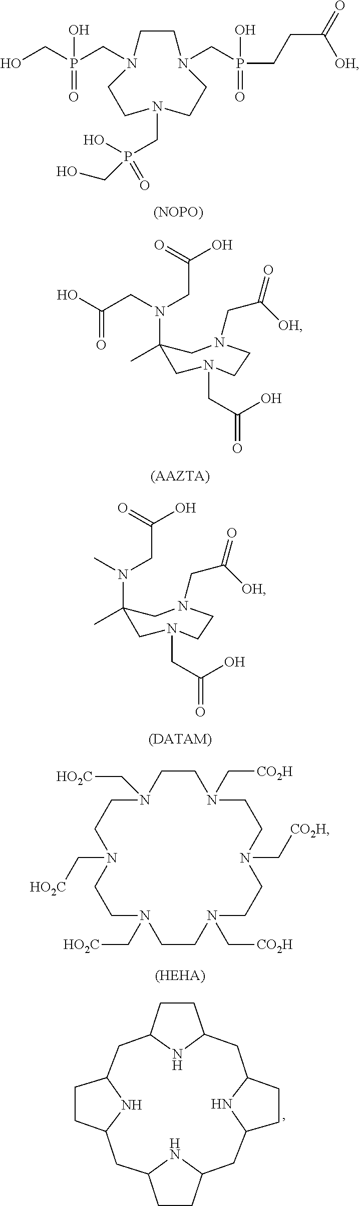

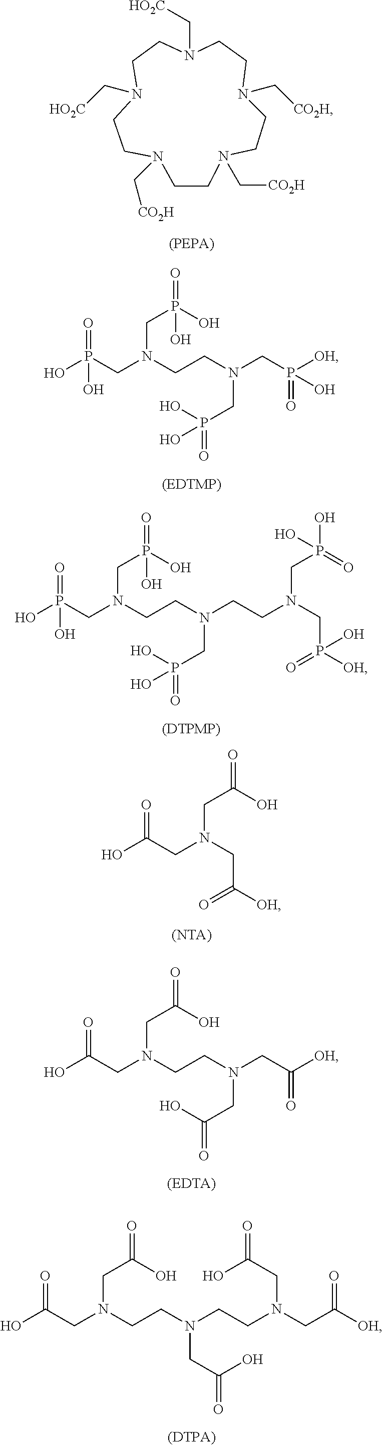 [SMILES: O=C(O)CN1CCN(CC(=O)O)CCN(CC(=O)O)CCN(CC(=O)O)CCN(CC(=O)O)CC1 ; O=P(O)(O)CN(CCN(CP(=O)(O)O)CP(=O)(O)O)CP(=O)(O)O ; O=P(O)(O)CN(CCN(CP(=O)(O)O)CP(=O)(O)O)CCN(CP(=O)(O)O)CP(=O)(O)O ; O=C(O)CN(CC(=O)O)CC(=O)O ; O=C(O)CN(CCN(CC(=O)O)CC(=O)O)CC(=O)O ; O=C(O)CN(CCN(CC(=O)O)CC(=O)O)CCN(CC(=O)O)CC(=O)O]

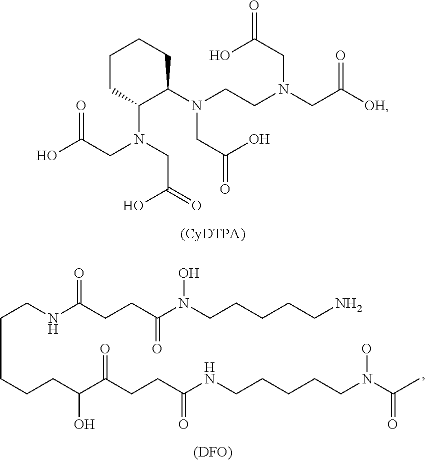

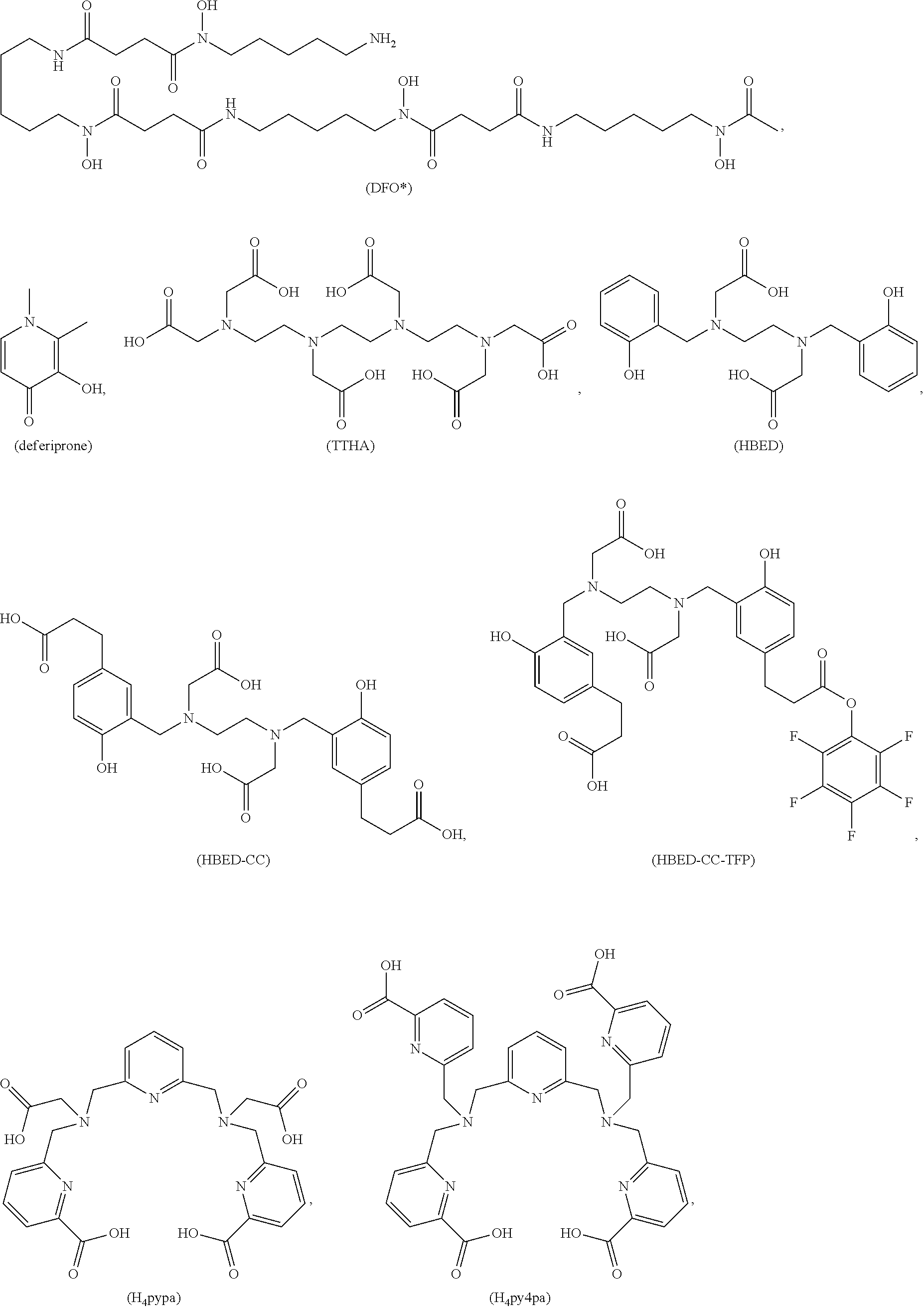

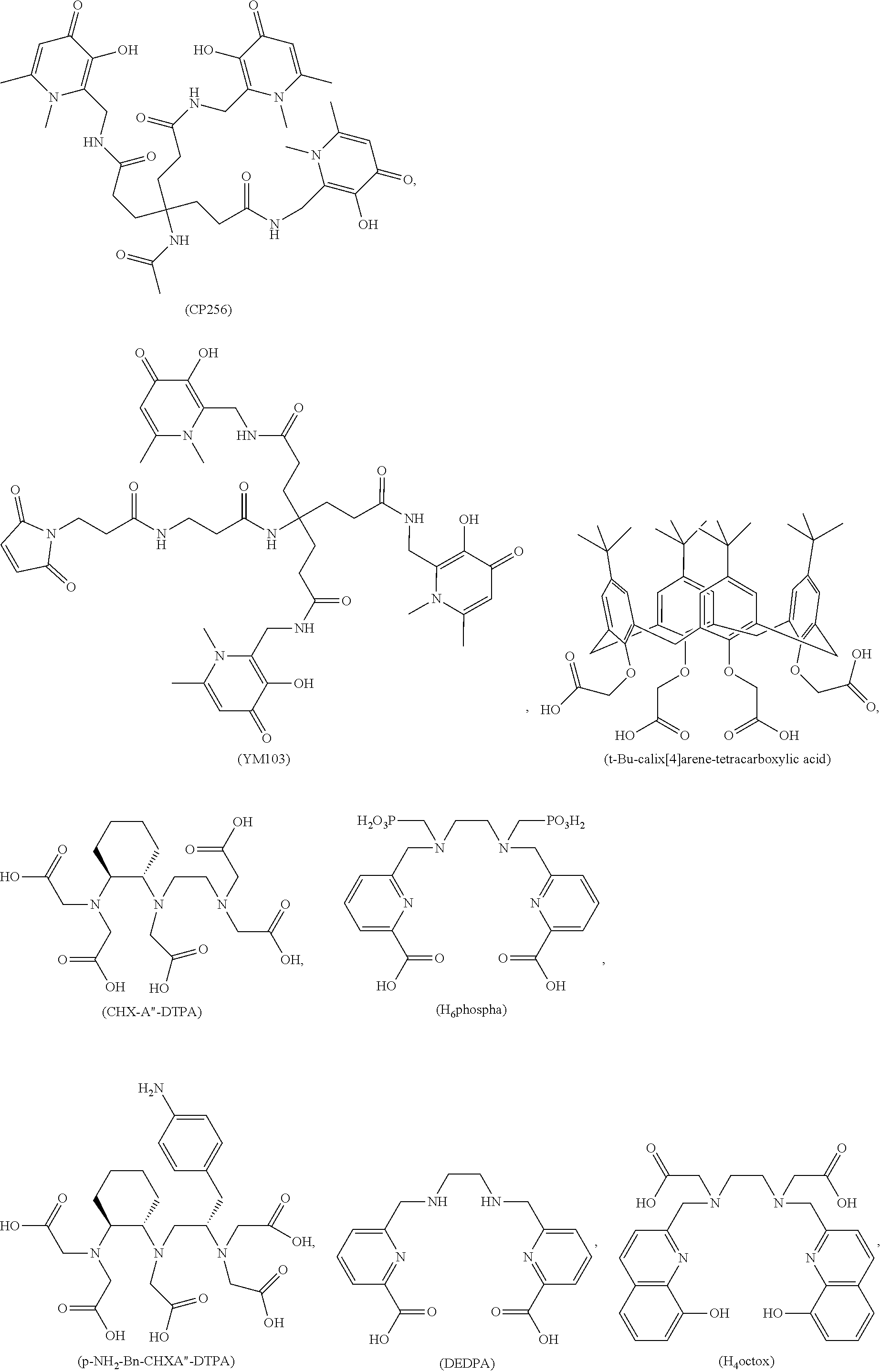

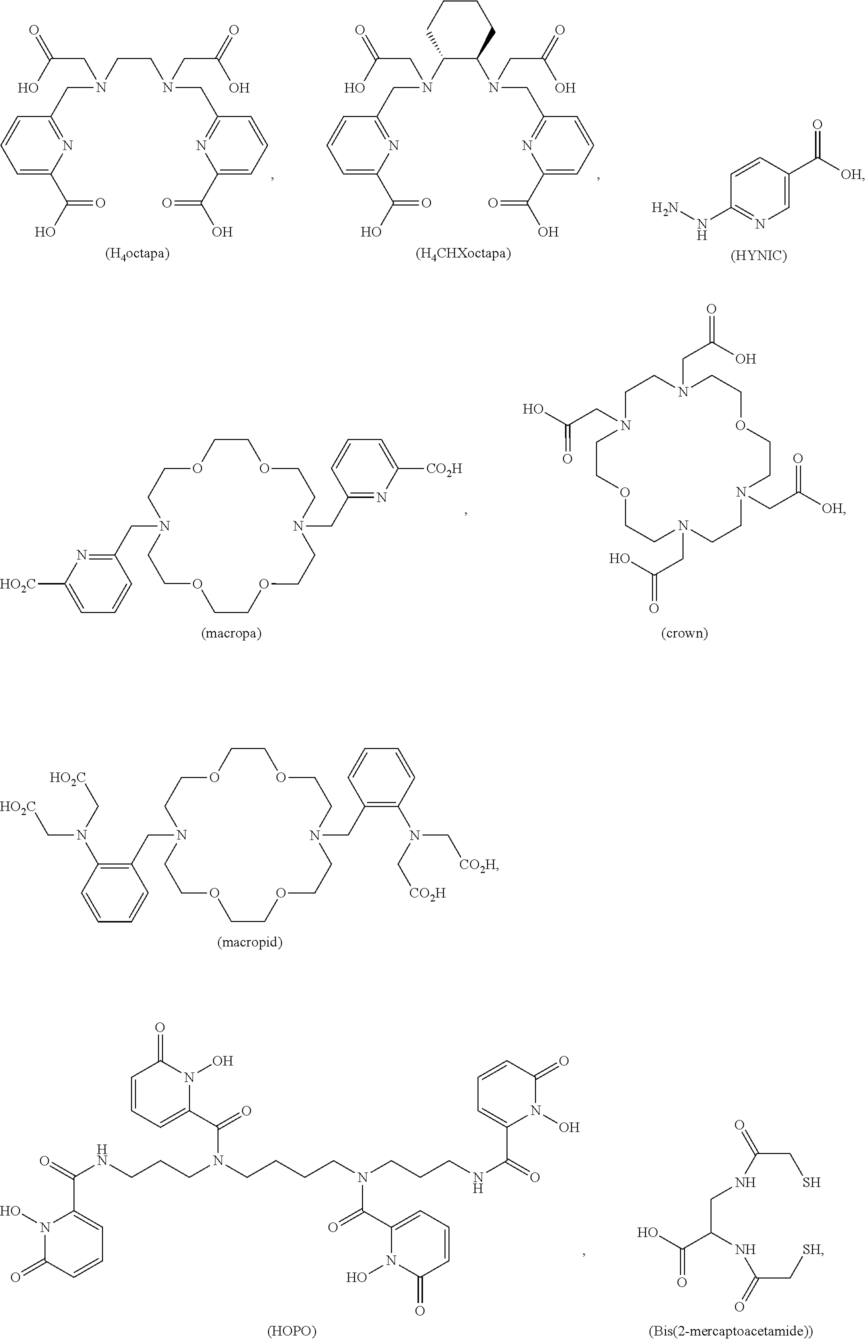

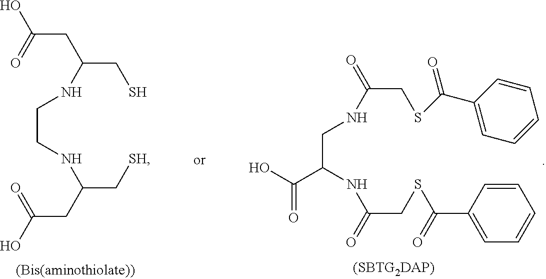 [SMILES: O=C(O)CC(CS)NCCNC(CS)CC(=O)O ; "or" ; O=C(CSC(=O)c1ccccc1)NCC(NC(=O)CSC(=O)c1ccccc1)C(=O)O]

In some embodiments, the chelating moiety R^{b} comprises a radionuclide and DOTA. In some embodiments, the chelating moiety R^{b} comprises a radionuclide and a DOTA derivative. In some embodiments, the chelating moiety comprises two independent chelators, and at least one or both are DOTA.

In some embodiments, the chelating moiety comprises a radionuclide and a chelator configured to bind the radionuclide (Z′), wherein the chelator comprises DOTA, DOTP, DOTMA, DOTAM, DTPA, NOTA, NTA, NODAGA, EDTA, DO3A, DO2A, NOC, TETA, CB-TE2A, DiAmSar, CB-Cyclam, DOTA-4AMP, H₄pypa, H₄octox, H₄octapa, p-NO₂—Bn-neunpa, or NOTP.

In some embodiments, the metal chelator described herein comprises macropa or crown. In some embodiments, the metal chelator described herein comprises macropa. In some embodiments, the metal chelator described herein comprises crown. In some embodiments, the metal chelator described herein comprises

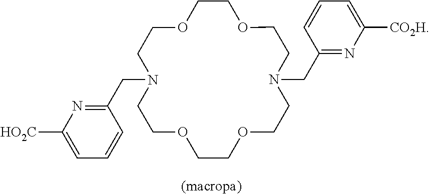 [SMILES: O=C(O)c1cccc(CN2CCOCCOCCN(Cc3cccc(C(=O)O)n3)CCOCCOCC2)n1]

In some embodiments, the metal chelator described herein comprises

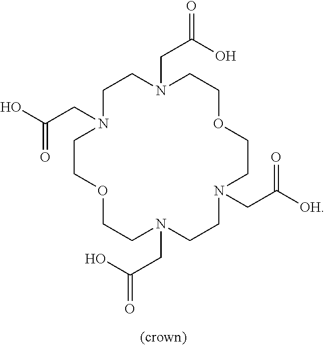 [SMILES: O=C(O)CN1CCOCCN(CC(=O)O)CCN(CC(=O)O)CCOCCN(CC(=O)O)CC1]

In some embodiments, the chelating moiety of R^{b} is independently selected from the group consisting of: cyclen, DO2A, DO3A, HP-DO3A, DO3A-Nprop, DO3AP, DO3AP^{PrA}, DO3AP^{ABn}, DO3AM^{nBu}, BT-DO3A, DOTA, DOTAGA, DOTA(GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE2A, TE2A, NOTA, NODAGA, NODA-MPAA, (3p-C-NETA), TACN, TACN-TM, NOTP, Sarcophagine (Sar), DiAmSar, SarAr, AmBaSar, cis-DO2A2P, trans-DO2A2P, DOTEP, p-NO₂—Bn-DOTA, BAT, DO3TMP-Monoamide, CHX-A″-DTPA, c-DEPA, PCTA, p-NO₂—Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa, CP256, THP, YM103, t-Bu-calix[4]arene-tetracarboxylic acid, CHX-A″-DTPA, H₆phospha, p-NH₂—Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), and SBTG₂DAP.

In some embodiments, R^{b} is a chelating moiety selected from the group consisting of: DOTA; 2,2′,2″-(10-(2-amino-2-oxoethyl)-1,4,7,10-tetraazacyclododecane-1,4,7-triyl)triacetic acid (PSC); DO3A; DO2A; DOTMA; DOTAM; DOTPA; 2,2′,2″-(10-(2-amino-2-oxoethyl)-1,4,7,10-tetraazacyclododecane-1,4,7-triyl)triacetic acid; Bn-DOTA; p-OH-Bn-DOTA; H₄pypa; H₄pypa-benzyl; H₄py4pa; H₄py4pa-benzyl; NOTA; macropa; crown; H₄octapa; H₄octapa-benzyl; and TTHA; or a radionuclide complex thereof.

In some embodiments, the chelating moiety of R^{b} is independently selected from the group consisting of: 1,4,7,10-tetraazacyclododecane-1,4,7,10-tetraacetic acid (DOTA);
1,4,7,10-tetraazacyclododecane-1,4,7-triacetic acid (DO3A);
1,4,7,10-tetraazacyclododecane-1,7-diacetic acid (DO2A);
α,α′,α″,α″′-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid;
benzyl-1,4,7,10-tetraazacyclododecane-1,4,7,10-tetraacetic acid (Bn-DOTA);
p-hydroxy-benzyl-1,4,7,10-tetraazacyclododecane-1,4,7,10-tetraacetic acid (p-OH-Bn-DOTA);
6,6′-(((pyridine-2,6-diylbis(methylene))bis((carboxymethyl)azanediyl))bis(methylene))dipicolinic acid (H₄pypa);
H₄pypa-benzyl;
6,6′,6″,6′″-(((pyridine-2,6-diylbis(methylene))bis(azanetriyl))tetrakis(methylene))-tetrapicolinic acid (H₄py4pa); H₄py4pa-benzyl;
2,2′,2″-(1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2,2′,2″,2″′-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H₄octapa-benzyl; and
3,6,9,12-tetrakis(carboxymethyl)-3,6,9,12-tetraazatetradecanedioic acid (TTHA);
   or a radionuclide complex thereof.

In some embodiments, the chelating moiety of R^{b} is independently selected from the group consisting of: DOTA; DO3A; DO2A; DOTMA; DOTAM; DOTPA; 2,2′,2″-(10-(2-amino-2-oxoethyl)-1,4,7,10-tetraazacyclododecane-1,4,7-triyl)triacetic acid; H₄pypa; H₄py4pa; NOTA; macropa; crown; H₄octapa; and TTHA; or a radionuclide complex thereof.

In some embodiments, R^{b} is DOTA or a radionuclide complex thereof. In some embodiments, R^{b} is DO3A or a radionuclide complex thereof. In some embodiments, R^{b} is DO2A or a radionuclide complex thereof. In some embodiments, R^{b} is DOTMA or a radionuclide complex thereof. In some embodiments, R^{b} is DOTAM or a radionuclide complex thereof. In some embodiments, R^{b} is DOTPA or a radionuclide complex thereof. In some embodiments, R^{b} is 2,2′,2″-(10-(2-amino-2-oxoethyl)-1,4,7,10-tetraazacyclododecane-1,4,7-triyl)triacetic acid or a radionuclide complex thereof. In some embodiments, R^{b} is H4pypa or a radionuclide complex thereof. In some embodiments, R^{b} is H₄py4pa or a radionuclide complex thereof. In some embodiments, R^{b} is NOTA or a radionuclide complex thereof. In some embodiments, R^{b} is macropa or a radionuclide complex thereof. In some embodiments, R^{b} is crown or a radionuclide complex thereof. In some embodiments, R^{b} is H₄octapa or a radionuclide complex thereof. In some embodiments, R^{b} is TTHA or a radionuclide complex thereof.

In some embodiments, R^{b} is: DOTA or DO3A; or a radionuclide complex thereof.

In some embodiments, the chelating moiety of R^{b} is independently selected from the group consisting of: DOTA; DO3A; DO2A; DOTMA; DOTAM; DOTPA; 2,2′,2″-(10-(2-amino-2-oxoethyl)-1,4,7,10-tetraazacyclododecane-1,4,7-triyl)triacetic acid; H₄pypa; H₄py4pa; NOTA; macropa; crown; H₄octapa; and TTHA; or a radionuclide complex thereof.

In some embodiments, R^{b} is DOTA or a radionuclide complex thereof. In some embodiments, R^{b} is DO3A or a radionuclide complex thereof. In some embodiments, R^{b} is DO2A or a radionuclide complex thereof. In some embodiments, R^{b} is DOTMA or a radionuclide complex thereof. In some embodiments, R^{b} is DOTAM or a radionuclide complex thereof. In some embodiments, R^{b} is DOTPA or a radionuclide complex thereof. In some embodiments, R^{b} is 2,2′,2″-(10-(2-amino-2-oxoethyl)-1,4,7,10-tetraazacyclododecane-1,4,7-triyl)triacetic acid or a radionuclide complex thereof. In some embodiments, R^{b} is H₄pypa or a radionuclide complex thereof. In some embodiments, R^{b} is H₄py4pa or a radionuclide complex thereof. In some embodiments, R^{b} is NOTA. In some embodiments, R^{b} is macropa. In some embodiments, R^{b} is crown. In some embodiments, R^{b} is I4octapa or a radionuclide complex thereof. In some embodiments, R^{b} is TTHA or a radionuclide complex thereof.

In some embodiments, the chelating moiety of R^{b} is: DOTA or DO3A; or a radionuclide complex thereof.

In some embodiments, R^{b} is a chelating moiety selected from the group consisting of:

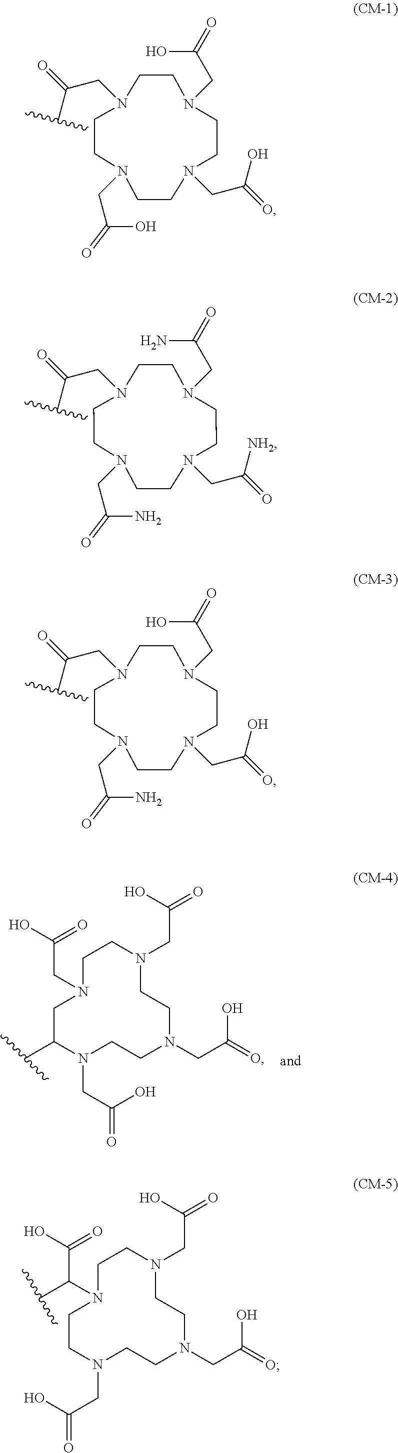 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, R^{b} is a chelating moiety selected from the group consisting of: (CM-1), (CM-2), (CM-4) and (CM-5); or a radionuclide complex thereof.

In some embodiments, R^{b} is

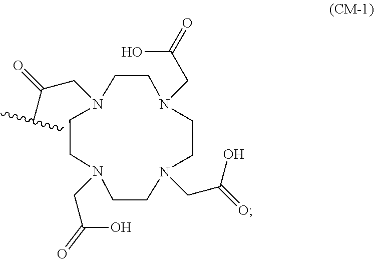 [SMILES: *C(=O)CN1CCN(CC(=O)O)CCN(CC(=O)O)CCN(CC(=O)O)CC1]

or a radionuclide complex thereof.

In some embodiments, R^{b} is: (CM-2), (CM-3), (CM-4), or (CM-5); or a radionuclide complex thereof.

In some embodiments, R^{b} is (CM-2), (CM-4), or (CM-5); or a radionuclide complex thereof.

In some embodiments, R^{b} is: (CM-2); or a radionuclide complex thereof. In some embodiments, R^{b} is: (CM-3); or a radionuclide complex thereof. In some embodiments, R^{b} is: (CM-5); or a radionuclide complex thereof.

In some embodiments, R^{b} is:

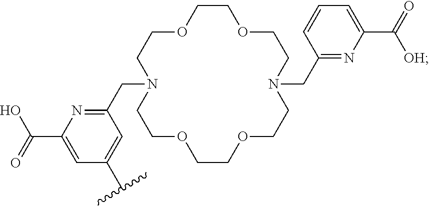 [SMILES: *c1cc(CN2CCOCCOCCN(Cc3cccc(C(=O)O)n3)CCOCCOCC2)nc(C(=O)O)c1]

or a radionuclide complex thereof. In some embodiments, R^{b} is:

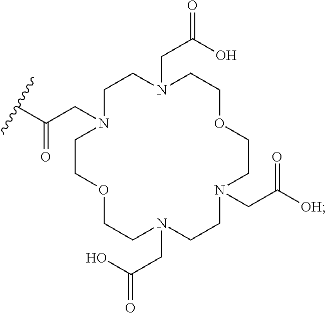 [SMILES: *C(=O)CN1CCOCCN(CC(=O)O)CCN(CC(=O)O)CCOCCN(CC(=O)O)CC1]

or a radionuclide complex thereof.

In some embodiments, R^{b} is:

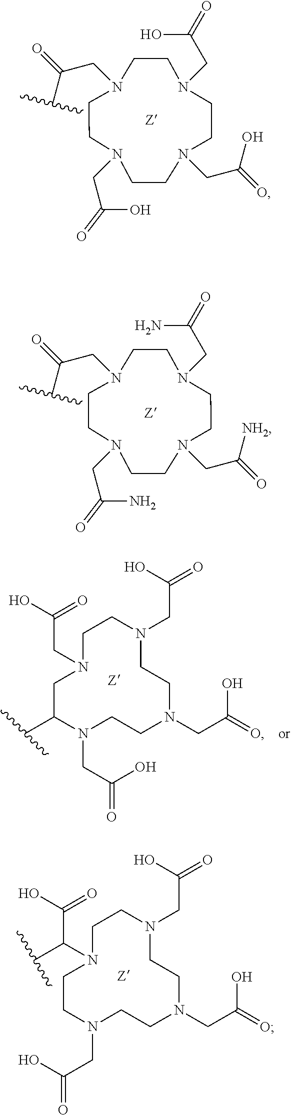

wherein Z′ is a diagnostic or therapeutic radionuclide.

In some embodiments, R^{b} is:

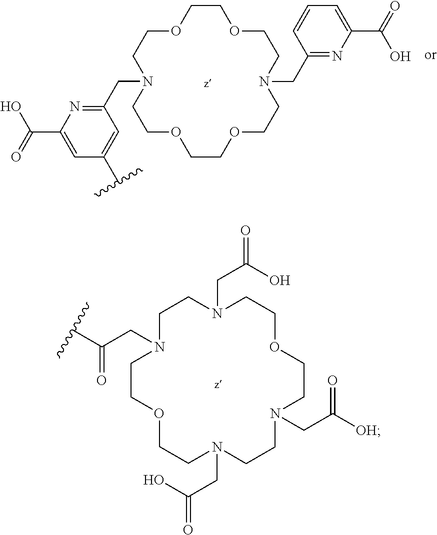

wherein Z′ is a diagnostic or therapeutic radionuclide.

In some embodiments, Z is a lanthanide or an actinide.

In some embodiments, Z′ is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

In some embodiments, Z is an Auger electron-emitting radionuclide, α-emitting radionuclide, β-emitting radionuclide, or γ-emitting radionuclide.

In some embodiments, Z′ is 111-indium (¹¹¹In), 115-indium (¹¹¹In), 67-gallium (⁶⁷Ga), 68-gallium (⁶⁸Ga), 69-gallium (⁶⁹Ga), 71-gallium (⁷¹Ga), 99m-technetium (^{99m}Tc), 195m-platinum (^{195m}Pt), 225-actinium (²²⁵Ac), 213-bismuth (²¹³Bi), 223-radium (²²³Ra), 211-astatine (²¹¹At), aluminium-[¹⁸F]fluoride ([¹⁸F]AlF), 90-yttrium (⁹⁰Y), 175-lutetium (¹⁷⁵Lu), 177-lutetium (¹⁷⁷Lu), 161-terbium (¹⁶¹Tb), 186-rhenium (¹⁸⁶Re), 188-rhenium (¹⁸⁸Re), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), 153-samarium (¹⁵³Sm), 89-strontium (⁸⁹Sr), 198-gold (¹⁹⁸Au), 169-erbium (¹⁶⁹Er), 165-dysprosium (¹⁶⁵Dy), 99m-technetium (^{99m}Tc), 89-zirconium (⁸⁹Zr), 52-manganese (⁵²Mn), 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra). In some embodiments, Z′ is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 211-astatine (²¹¹At), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

In some embodiments, Z′ is aluminium-[¹⁸F]fluoride ([¹⁸F]AlF), 111-indium (¹¹¹In), 115-indium (¹⁵¹In), 90-yttrium (⁹¹Y), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), or 213-bismuth (²¹³Bi).

In some embodiments, Z′ is 90-yttrium (⁹⁰Y), 67-gallium (⁶⁷Ga), 68-gallium (⁶⁸Ga), 225-actinium (²²⁵Ac), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), or 213-bismuth (²¹³Bi).

In some embodiments, Z′ is 225-actinium (²²⁵Ac), 177-lutetium (¹⁷⁷Lu), 161-terbium (¹⁶¹Tb), 204-lead (²⁰⁴Pb), 212-lead (²¹²Pb), 67-copper (⁶⁷Cu), 90-yttrium (⁹⁰Y), or 213-bismuth (²¹³Bi).

In some embodiments, Z′ is an Auger electron-emitting radionuclide that is 111-indium (¹¹¹In), 67-gallium (⁶⁷Ga), 99m-technetium (^{99m}Tc), or 195m-platinum (^{195m}Pt); or an α-emitting radionuclide that is 225-actinium (²²⁵Ac), 213-bismuth (²¹³Bi), 223-radium (²²³Ra), or 211-astatine (²¹¹At); or a β-emitting radionuclide that is aluminium-[¹⁸F]fluoride ([¹⁸F]AlF), 68-gallium (⁶⁸Ga), 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), 52-manganese (⁵²Mn), or 212-lead (²¹²Pb); or a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), 64-copper (⁶⁴Cu), or 195m-platinum (^{195m}Pt).

In some embodiments, Z′ is an α-emitting radionuclide that is 225-actinium (²²⁵Ac), 213-bismuth (²¹³Bi), 223-radium (²²³Ra), or 212-lead (²¹²Pb). In some embodiments, Z′ is α-emitting radionuclide that is 90-yttrium (⁹⁰Y), 177-lutetium (¹⁷⁷Lu), iodine-131 (¹³¹I), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn).

In some embodiments, Z′ is a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, R^{b} comprises a radionuclide (Z′) and a chelator configured to bind the radionuclide (Z), wherein the radionuclide is suitable for positron emission tomography (PET) analysis, single-photon emission computerized tomography (SPECT), or magnetic resonance imaging (MRI). In some embodiments, R^{b} comprises a radionuclide (Z) and a chelator configured to bind the radionuclide (Z′), wherein the radionuclide is suitable for positron emission tomography (PET) analysis or single-photon emission computerized tomography (SPECT). In some embodiments, the radionuclide is copper-64 (⁶⁴Cu), gallium-68 (⁶⁸Ga), 111-indium (¹¹¹In), or technetium-99m (^{99m}Tc).

### Metals (Radionuclides)

In some embodiments, Z′ is an Auger electron-emitting radionuclide. In some embodiments, Z′ is an α-emitting radionuclide. In some embodiments, Z′ is a β-emitting radionuclide. In some embodiments, Z′ is a γ-emitting radionuclide. In some embodiments, the type of radionuclide used in a peptide targeted therapeutic compound can be tailored to the specific type of cancer, the type of targeting moiety (e.g., peptide ligand), etc. Radionuclides that undergo α-decay emit α-particles (helium ions with a ±2 charge) from their nuclei. As a result of α-decay the daughter nuclide has 2 protons less and 2 neutrons less than the parent nuclide. This means that in α-decay, the proton number is reduced by 2 while the nucleon number is reduced by 4. Radionuclides that undergo β-decay emit β-particles (electrons) from their nuclei. During β-decay, one of the neutrons changes into a proton and an electron. The proton remains in the nucleus while the electron is emitted as a β-particle. This means that in β-decay, the nucleus loses a neutron but gains a proton. In γ-decay, a nucleus in an excited state (higher energy state) emits a γ-ray photon to change to a lower energy state. There is no change in the proton number and nucleon number during the γ-decay. The emission of γ-rays often accompanies the emission of α-particles and β-particles.

Auger electrons (AEs) are very low energy electrons that are emitted by radionuclides that decay by electron capture (EC) (e.g. ¹¹¹In, ⁶⁷Ga, ^{99m}Tc, ^{195m}Pt, ¹²⁵I and ¹²³I). This energy is deposited over nanometer-micrometer distances, resulting in high linear energy transfer that is potent for causing lethal damage in cancer cells. Thus, AE-emitting radiotherapeutic agents have great potential for treatment of cancer.

β-Particles are electrons emitted from the nucleus. They typically have a longer range in tissue (of the order of 1-5 mm) and are the most frequently used.

α-Particles are helium nuclei (two protons and two neutrons) that are emitted from the nucleus of a radioactive atom. Depending on their emission energy, they can travel 50-100 μm in tissue. They are positively charged and are orders of magnitude larger than electrons. The amount of energy deposited per path length travelled (designated ‘linear energy transfer’) of α-particles is approximately 400 times greater than that of electrons. This leads to substantially more damage along their path than that caused by electrons. An ca-particle track leads to a preponderance of complex and largely irreparable DNA double-strand breaks. The absorbed dose required to achieve cytotoxicity relates to the number of α-particles traversing the cell nucleus. With use of this as a measure, cytotoxicity may be achieved with a range of 1 to 20 α-particle traversals of the cell nucleus. The resulting high potency, combined with the short range of α-particles (which reduces normal organ toxicity), has led to substantial interest in developing α-particle-emitting agents. The α-particle emitters typically used include bismuth-212, lead-212, bismuth-213, actinium-225, radium-223 and thorium-227.

In some embodiments, Z′ is a diagnostic or therapeutic radionuclide.

### Representative Radionuclides

| | Radionuclide | | |
|---|---|---|---|
| Isotope | t_{1/2} (h) | Decay mode | |
| ⁶⁰Cu | 0.4 | β+ (93%), EC (7%) | |
| ⁶¹Cu | 3.3 | β+ (62%), EC (38%) | |
| ⁶²Cu | 0.16 | β+ (98%), EC (2%) | |
| ⁶⁴Cu | 12.7 | β+ (19%), EC (41%), β− (40%) | |
| ⁶⁷Cu | 61.9 | | |
| ⁶⁶Ga | 9.5 | β+ (56%), EC (44%) | |
| ⁶⁷Ga | 78.2 | EC | (100%) |
| ⁶⁸Ga | 1.1 | β+ (90%), EC (10%) | |
| ⁴⁴Sc | 3.9 | β+ (94%), EC (6%) | |
| ⁴⁷Sc | 80.2 | β− | (100%) |
| ¹¹¹In | 67.2 | EC | (100%) |
| ^{114m}In | 49.5 d | EC | (100%) |
| ¹¹⁴In (daughter) | 73 s | β− | (100%) |
| ¹⁶¹Tb | 166.8 | β− | (100%) |
| ¹⁷⁷Lu | 159.4 | β− | (100%) |
| ⁸⁶Y | 14.7 | β+ (33%), EC (66%) | |
| ⁹⁰Y | 64.1 | β− | (100%) |
| ⁸⁹Zr | 78.5 | β+ (23%), EC (77%) | |
| ²¹²Bi | 1.1 | α (36%), β− (64%) | |
| ²¹³Bi | 0.76 | α (2.2%), β− (97.8%) | |
| ²¹²Pb (daughter is ²¹²Bi) | 10.6 | β− | (100%) |
| ²²⁵Ac | 240 | α | (100%) |

| | | |
|---|---|---|
| ²²⁷Th | 448.8 | α |
| ²¹¹At | 7.2 | α |

In some embodiments, Z′ is an Auger electron-emitting radionuclide. In some embodiments, Z′ is an Auger electron-emitting radionuclide that is 111-indium (¹¹¹In), 67-gallium (⁶⁷Ga), 68-gallium (⁶⁸Ga), 99m-technetium (^{991m}Tc), or 195m-platinum (^{195m}Pt).

In some embodiments, Z′ is an α-emitting radionuclide. In some embodiments, Z′ is an α-emitting radionuclide that is 225-actinium (²²⁵Ac), 213-bismuth (²¹³Bi), 223-radium (²²³1Ra), or 212-lead (²¹²Pb).

In some embodiments, Z′ is a j-emitting radionuclide. In some embodiments, Z′ is a (3-emitting radionuclide that is aluminium-[¹⁸F]fluoride ([¹⁸F]AlF), 68-gallium (⁶⁸Ga), 90-yttrium (⁹⁰Y), 177-lutetium (¹⁷⁷Lu), 161-terbium (¹⁶¹Tb), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn), or 212-lead (²¹²Pb).

In some embodiments, Z′ is a γ-emitting radionuclide. In some embodiments, Z′ is a 7-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is an Auger electron-emitting radionuclide that is 111-indium (¹¹¹In), 67-gallium (⁶⁷Ga), 99m-technetium (^{991m}Tc), or 195m-platinum (^{195m}Pt); or Z′ is an α-emitting radionuclide that is 225-actinium (²²⁵Ac), 213-bismuth (²¹³Bi), 223-radium (²²³Ra), or 212-lead (²¹²Pb); or V is a β-emitting radionuclide that is aluminium-[¹⁸F]fluoride ([¹⁸F]AlF), 68-gallium (⁶⁸Ga), 90-yttrium (⁹⁰Y), 177-lutetium (¹⁷⁷Lu), 161-terbium (¹⁶¹Tb), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr) 198-gold (¹⁹⁸Au, 169-erbium (¹⁶⁹Er), 165-dysprosium (¹⁶⁵Dy), 99m-technetium (^{99m}Tc), 89-zirconium (⁸⁹Zr), or 52-manganese (⁵²Mn); or Z′ is a 7-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

In some embodiments, Z′ is ⁹⁴Tc, ⁹³In, ¹¹¹In, ⁶⁷Ga, ⁶⁸Ga, ⁸⁶Y, ⁹⁰Y, ¹⁷⁷Lu, ¹⁶¹Tb, ¹⁸⁶Re, ¹⁸⁸Re, ⁶⁴Cu, ⁶⁷Cu, ⁵⁵Co, ⁵⁷Co, ⁴³Sc, ⁴⁴Sc, ⁴⁷Sc, ²²⁵Ac, ²¹³Bi, ²¹²Bi, ²¹²Pb, ²²⁷Th, ¹⁵³Sm, ¹⁶⁶Ho, ¹⁵²Gd, ¹⁵³Gd, ¹⁵⁷Gd, and ¹⁶⁶Dy.

In some embodiments, Z′ is ⁶⁷Cu, ⁶⁴Cu, ⁹⁰Y ¹⁰⁹Pd, ¹¹¹Ag, ¹⁴⁹Pm, ¹⁵³Sn, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ⁴⁴Sc, ⁴⁷Sc, ⁷⁰As, ⁷¹As, ⁷²As, ⁷³As, ⁷⁴As, ⁷⁶As, ⁷⁷As, ²¹²Pb, ²¹²Bi, ²¹³Bi, ²²⁵Ac, ^{117m}Sn, ⁶⁷Ga, ²⁰¹Tl, ¹⁶⁰Gd, ¹⁴⁸Nd, and ⁸⁹Sr.

In some embodiments, Z′ is ⁶⁸Ga, ⁴³S, ⁴⁴Sc, ⁴⁷Sc, ¹⁷⁷Lu, ¹⁶¹Tb, ²²⁵Ac, ²¹³Bi, ²¹²Bi, or ²¹²Pb. In some embodiments, Z′ is ⁶⁷Ga, ^{99m}Tc, ¹¹¹In, or ²⁰¹Tl.

In some embodiments, the radionuclide (Z′) is ⁴⁴Sc, ⁶⁴Cu, ⁶⁷Ga, ⁶⁸Ga, ⁸⁶Y, ⁸⁹Zr, ^{99m}Tc ¹¹¹In, or ¹⁷⁷Lu.

In some embodiments, Z′ is ⁴⁴Sc, ⁶⁴Cu, ⁶⁸Ga, ⁸⁶Y, or ⁸⁹Zr. In some embodiments, Z′ is ⁶⁷Ga, ^{99m}Tc, ¹¹¹In, or ¹⁷⁷Lu.

In some embodiments, Z′ is ⁶⁷Cu, ⁹⁰Y, ¹¹¹In, ¹⁷⁷Lu, ²²⁵Ac, ²¹²Pb, or ²¹³Bi.

In some embodiments, Z′ is aluminium-[¹⁸F]fluoride ([¹⁸F]AlF), 111-indium (¹¹¹In), 115-indium (¹¹⁵In), 90-yttrium (⁹⁰Y), 67-gallium (⁶⁷Ga), 68-gallium (⁶⁸Ga), 69-gallium (⁶⁹Ga) 71-gallium (⁷¹Ga), 225-actinium (²²⁵Ac), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), or 213-bismuth (²¹³Bi).

In some embodiments, the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 99m-technetium (^{99m}Tc), 195m-platinum (^{195m}Pt), 225-actinium (²²⁵Ac), 213-bismuth (²¹³Bi), 223-radium (²²³Ra), 211-astatine (²¹¹At), aluminium-[¹⁸F]fluoride ([¹⁸F]AlF), 90-yttrium (⁹⁰Y), 175-lutetium (¹⁷⁵Lu), 177-lutetium (¹⁷⁷Lu), 161-terbium (¹⁶¹Tb), 186-rhenium (¹⁸⁶Re), 188-rhenium (¹⁸⁸Re), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), 153-samarium (¹⁵³Sm), 89-strontium (⁸⁹Sr), 198-gold (¹⁹⁸Au), 169-erbium (¹⁶⁹Er), 165-dysprosium (¹⁶⁵Dy), 99m-technetium (^{99m}Tc), 89-zirconium (⁸⁹Zr), 52-manganese (⁵²Mn), 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra). In some embodiments, the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 211-astatine (²¹¹At), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

In some embodiments, the radionuclide of the radionuclide complex is:
an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt); or
an α-emitting radionuclide that is 225-actinium (²²⁵Ac), 213-bismuth (²¹³Bi), 223-radium (²²³Ra), or 212-lead (²¹²Pb); or
a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or
a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, Z′ is aluminium-[¹⁸F]fluoride ([¹⁸F]AlF), 111-indium (¹¹¹In), 115-indium (¹¹⁵In), 90-yttrium (⁹⁰Y), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), or 213-bismuth (²¹³Bi).

In some embodiments, Z′ is 90-yttrium (⁹⁰Y), 67-gallium (⁶⁷Ga), 68-gallium (⁶⁸Ga), 225-actinium (²²⁵Ac), 175-lutetium (¹⁷⁵Lu), 177-lutetium (¹⁷⁷Lu), 161-terbium (¹⁶¹Tb), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), or 213-bismuth (²¹³Bi).

In some embodiments, Z′ 225-actinium (²²⁵Ac), 177-lutetium (¹⁷⁷Lu), 161-terbium (¹⁶¹Tb), 212-lead (²¹²Pb), 67-copper (⁶⁷Cu), 90-yttrium (⁹⁰Y), or 213-bismuth (²¹³Bi).

In some embodiments, Z′ is 111-indium (¹¹¹In). In some embodiments, Z′ is 115-indium (¹¹⁵In). In some embodiments, Z′ is aluminium-[¹⁸F]fluoride ([¹⁸F]AlF). In some embodiments, Z′ is 67-gallium (⁶⁷Ga). In some embodiments, Z′ is 68-gallium (⁶⁸Ga). In some embodiments, Z′ is 69-gallium (⁶⁹Ga), 71-gallium (⁷¹Ga), or a mixture thereof. In some embodiments, Z′ is 225-actinium (²²⁵Ac). In some embodiments, Z′ is 175-lutetium (¹⁷⁵Lu). In some embodiments, Z′ is 177-lutetium (¹⁷⁷Lu). In some embodiments, Z′ is 161-terbium (¹⁶¹Tb). In some embodiments, Z′ is 204-lead (²⁰⁴Pb), 206-lead (²⁰⁶Pb), 207-lead (²⁰⁷Pb), 208-lead (²⁰⁸Pb), or a mixture thereof. In some embodiments, Z′ is 212-lead (²¹²Pb). In some embodiments, Z′ is 64-copper (⁶⁴Cu). In some embodiments, Z′ is 63-copper (⁶³Cu), 65-copper (⁶⁵Cu), or a mixture thereof. In some embodiments, Z′ is 67-copper (⁶⁷Cu). In some embodiments, Z′ is 213-bismuth (²¹³Bi). In some embodiments, Z′ is 211-astatine (²¹¹At).

In some embodiments, Z′ is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu) or 177-lutetium (¹⁷⁷Lu).

### Exemplary Chelator and Radionuclide Complexes

Radionuclides have useful emission properties that can be used for diagnostic imaging techniques, such as single photon emission computed tomography (SPECT, e.g. ⁶⁷Ga, ^{99m}Tc, ¹¹¹In, ¹⁷⁷Lu) and positron emission tomography (PET, e.g. ⁶⁸Ga, ⁶⁴Cu, ⁴⁴Sc, ⁸⁶Y, ⁸⁹Zr), as well as therapeutic applications (e.g. ⁴⁷Sc, ¹¹⁴mIn, ¹⁷⁷Lu, ⁹⁰Y, ^{212/213}Bi, ²¹²Pb, ²²⁵Ac, ^{186/188}Re). A fundamental component of a radiometal-based radiopharmaceutical is the chelator, the ligand system that binds the radiometal ion in a tight stable coordination complex so that it can be properly directed to a desirable molecular target in vivo. Guidance for selecting the optimal match between chelator and radiometal for a particular use is provided in the art (e.g., see Price et al., “Matching chelators to radiometals for radiopharmaceuticals”, Chem. Soc. Rev., 2014, 43, 260-290).

In some embodiments, R^{b} is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H4py4pa; H4py4pa-benzyl; H4octapa; H4octapa-benzyl; and TTHA; or a radionuclide complex thereof.

In some embodiments, R^{b} is:

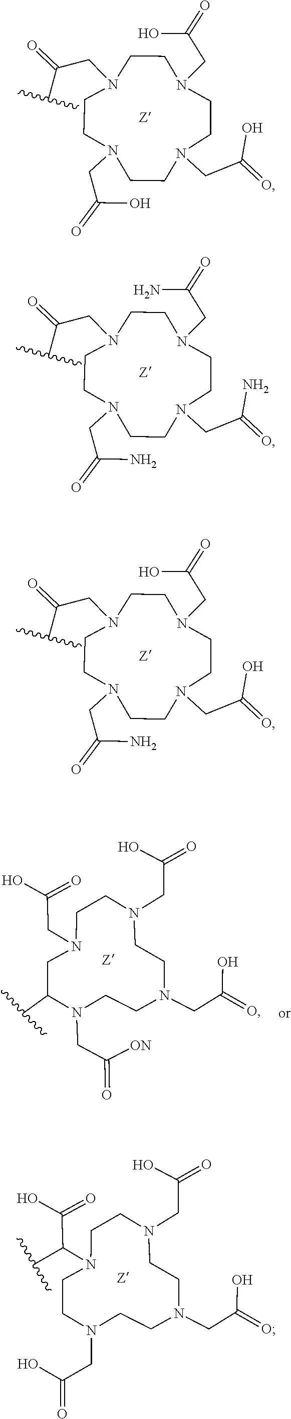

wherein Z′ is a diagnostic or therapeutic radionuclide.

In some embodiments, the radionuclide (Z′) is ⁴⁴Sc, ⁶⁴Cu, ⁶⁷Ga, ⁶⁸Ga, ⁸⁶Y, ⁸⁹Zr, ^{99m}Tc, ¹¹¹In, or ¹⁷⁷Lu. In some embodiments, the radionuclide (Z′) is ⁴⁴Sc, ⁶⁴Cu, ⁶⁸Ga, ⁸⁶Y, or ⁸⁹Zr. In some embodiments, the radionuclide (Z′) is ⁶⁷Ga, ^{99m}Tc, ¹¹¹In, or ¹⁷⁷Lu.

In some embodiments, the radionuclide (Z′) is ⁶⁷Cu, ⁹⁰Y, ¹¹¹In, ¹⁷⁷Lu, ²²⁵Ac, ²¹²Pb, or ²¹³Bi.

In some embodiments, the radionuclide (Z′) is 111-indium (¹¹¹In), 115-indium (¹¹⁵In), aluminium-[¹⁸F]fluoride ([¹⁸F]AlF), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 161-terbium (¹⁶¹Tb), 206-lead (²⁰⁶Pb), 207-lead (²⁰⁷Pb), 208-lead (²⁰⁸Pb), 212-lead (²¹²Pb), 60-copper (⁶⁰Cu), 61-copper (⁶¹Cu), 62-copper (⁶²Cu), 63-copper (⁶³Cu), 64-copper (⁶⁴Cu), 65-copper (⁶⁵Cu), or 67-copper (⁶⁷Cu).

In some embodiments, the radionuclide (Z′) is 111-indium (¹¹¹In) or 115-indium (¹¹⁵In), or a mixture thereof. In some embodiments, the radionuclide (Z′) is aluminium-[¹⁸F]fluoride ([¹⁸F]AlF). In some embodiments, the radionuclide (Z′) is 67-gallium (⁶⁷Ga), 68-gallium (⁶⁸Ga), 69-gallium (⁶⁹Ga), or 71-gallium (⁷¹Ga), or a mixture thereof. In some embodiments, the radionuclide (Z′) is 225-actinium (²²⁵Ac). In some embodiments, the radionuclide (Z′) is 175-lutetium (¹⁷⁵Lu) or 177-lutetium (¹⁷⁷Lu), or a mixture thereof. In some embodiments, the radionuclide (Z′) is 161-terbium (¹⁶¹Tb). In some embodiments, the radionuclide (Z′) is 206-lead (²⁰⁶Pb), 207-lead (²⁰⁷Pb), 208-lead (²⁰⁸Pb), or 212-lead (²¹²Pb), or a mixture thereof. In some embodiments, the radionuclide (Z′) is 60-copper (⁶⁰Cu), 61-copper (⁶¹Cu), 62-copper (⁶²Cu), 63-copper (⁶³Cu), 64-copper (⁶⁴Cu), 65-copper (⁶⁵Cu), or 67-copper (⁶⁷Cu), or a mixture thereof.

In some embodiments, the radionuclide (Z′) is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu) or 177-lutetium (¹⁷⁷Lu).

In some embodiments, the radionuclide (Z′) is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

### Emission Tomography

In some embodiments, R^{b} comprises a chelated radionuclide that is suitable for positron emission tomography (PET) analysis or single-photon emission computerized tomography (SPECT). In some embodiments, R^{b} comprises a chelated radionuclide that is suitable for single-photon emission computerized tomography (SPECT). In some embodiments, R^{b} comprises a chelated radionuclide that is suitable for positron emission tomography (PET) analysis. In some embodiments, R^{b} comprises a chelated radionuclide that is suitable for positron emission tomography imaging, positron emission tomography with computed tomography imaging, or positron emission tomography with magnetic resonance imaging (MRI).

In some embodiments, R^{b} is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H₄py4pa; H4py4pa-benzyl; H4octapa; H4octapa-benzyl; and TTHA; or a radionuclide complex thereof. In some embodiments, the radionuclide is copper-64 (⁶⁴Cu), gallium-68 (⁶⁸Ga), or technetium-99m (^{99m}Tc).

In some embodiments, a conjugate described herein is designed to have a prescribed elimination profile. The elimination profile can be designed by adjusting the sequence and length of the peptide ligand, the property of the linker, the type of radionuclide, etc. In some embodiments, the conjugate has an elimination half-life of about 5 minutes to about 12 hours. In some embodiments, the conjugate has an elimination half-life of about 10 minutes to about 8 hours. In some embodiments, the conjugate has an elimination half-life of at least about 15 minutes, at least about 30 minutes, at least about 1 hour, at least about 2 hours, at least about 3 hours, at least about 4 hours, at least about 5 hours, at least about 6 hours, or at least about 8 hours. In some embodiments, the conjugate has an elimination half-life of at most about 15 minutes, at most about 30 minutes, at most about 1 hour, at most about 2 hours, at most about 3 hours, at most about 4 hours, at most about 5 hours, at most about 6 hours, or at most about 8 hours. In some embodiments, the elimination half-life is determined in rats. In some embodiments, the elimination half-life is determined in humans.

A herein described conjugate can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is about 15 minutes to about 1 day. In some embodiments, the elimination half-life of the conjugate in a tumor is at least 1.1, at least 1.2, at least 1.3, at least 1.4, at least 1.5, at least 2.0, at least 2.5, at least 3.0, at least 4.0, or at least 5.0-fold of the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the pharmaceutical in the living system. In some embodiments, the elimination half-life is measured as effective half-life, which is the half-life of a radiopharmaceutical in a living system taking into account the half-life of the radionuclide.

Response and toxicity prediction is essential for the rational implementation of cancer therapy. The biological effects of radionuclide therapy are mediated by a well-defined physical quantity, the absorbed dose (D), which is defined as the energy absorbed per unit mass of tissue.

Radiation dosimetry is the measurement, calculation and assessment of the ionizing radiation dose absorbed by an object, usually the human body, and may be thought of as the ability to perform the equivalent of a pharmacodynamic study in treated patients in real time. This applies both internally, due to ingested or inhaled radioactive substances, or externally due to irradiation by sources of radiation. Dosimetry analysis may be performed as part of patient treatment to calculate tumor versus normal organ absorbed dose and therefore the likelihood of treatment success.

A conjugate described herein can have a prescribed time-integrated activity coefficient (i.e., ã) in a tumor or non-tumor tissues of a subject. As used herein, ã represents the cumulative number of nuclear transformations occurring in a source tissue over a dose-integration period per unit administered activity. The ã value of a conjugate can be tuned by modifications of the non-peptide small molecule drug conjugate (NPDC). The ã value can be determined using a method known in the art. In some embodiments, the a value of the conjugate in a tumor is from about 10 minutes to about 1 day. The ã value of the conjugate in a tumor can be the same as the ã value of the conjugate in a non-tumor tissue of the subject. The ã value of the conjugate in a tumor can be longer or shorter than the ã value of the conjugate in a non-tumor tissue of the subject. In some embodiments, the ã value of the conjugate in a tumor is at least 1.1, at least 1.2, at least 1.3, at least 1.4, at least 1.5, at least 2.0, at least 2.5, at least 3.0, at least 4.0, or at least 5.0-fold of the ã value of the conjugate in a non-tumor tissue of the subject.

A conjugate described herein can have an ã value in an organ of a subject. In some embodiments, the conjugate has an a value in a kidney of the subject of at most 24 hours. In some embodiments, the ã value of the conjugate in a kidney of the subject is at most 18 hours, 15 hours, 12 hours, 10 hours, 8 hours, 6 hours, or 5 hours. In some embodiments, the a value of the conjugate in a kidney of the subject is about 30 minutes to about 24 hours. In some embodiments, the a value of the conjugate in a kidney of the subject is about 2 to 24 hours. In some embodiments, the ã value of the conjugate in a kidney of the subject is more than 24 hours. In some embodiments, the ã value of the conjugate in a liver of the subject is at most 24 hours. In some embodiments, the a value of the conjugate in a liver of the subject is at most 18 hours, 15 hours, 12 hours, 10 hours, 8 hours, 6 hours, or 5 hours. In some embodiments, the a value of the conjugate in a liver of the subject is about 30 minutes to about 24 hours. In some embodiments, the ã value of the conjugate in a liver of the subject is about 2 to 24 hours. In some embodiments, the ã value of the conjugate in a liver of the subject is more than 24 hours.

### Linkers

In some embodiments, the linker has a prescribed length thereby linking the Protease-Activated Receptor-2 (PAR2) targeting ligand and the chelating moiety or a radionuclide complex thereof (R^{b}) while allowing an appropriate distance therebetween.

For the linkers described herein, no orientation of the linker is implied by the direction in which the formula of the linker is written. For example, the formula

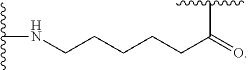 [SMILES: *NCCCCCC(*)=O]

represents both

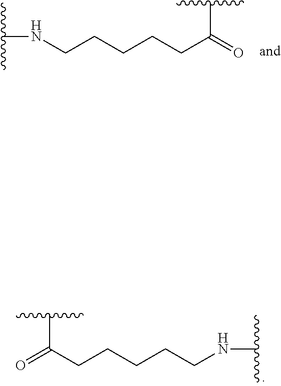 [SMILES: *NCCCCCC(*)=O ; *NCCCCCC(*)=O]

Additionally, the formula

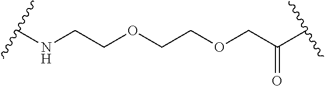 [SMILES: *NCCOCCOCC(*)=O]

represents both

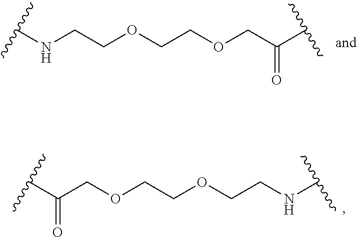 [SMILES: *NCCOCCOCC(*)=O ; *NCCOCCOCC(*)=O]

and the formula

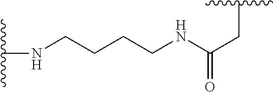 [SMILES: *CC(=O)NCCCCN*]

represents both

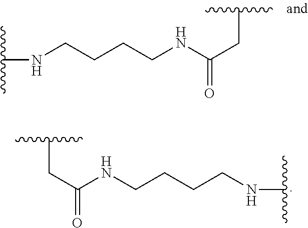 [SMILES: *CC(=O)NCCCCN* ; *CC(=O)NCCCCN*]

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker comprises one or more linear structures, one or more non-linear structures, one or more branched structures, one or more cyclic structures, one or more flexible moieties, one or more rigid moieties, or combinations thereof.

In some embodiments, a linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids.

In some embodiments, the linker comprises a peptide linkage. The peptide linkage comprises L-amino acids and/or D-amino acids. In some embodiments, D-amino acids are preferred in order to minimize immunogenicity and nonspecific cleavage by background peptidases or proteases.

In some embodiments, a linker has 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 10 atoms in length. In some embodiments, the linker has 1 to 20 atoms in length.

In some embodiments, a linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences, proline-rich sequences, and regions rich in double and/or triple bonds.

In some embodiments, the linker comprises a click chemistry residue. In some embodiments, the linker is attached to a peptide ligand, to a metal chelator or both via click chemistry. For example, in some embodiments, a peptide ligand comprises an azide group that reacts with an alkyne moiety of the linker. For another example, in some embodiments, a peptide ligand comprises an alkyne group that reacts with an azide of the linker. The metal chelator and the linker can be attached similarly. In some embodiments, the linker comprises an azide moiety, an alkyne moiety, or both. In some embodiments, the linker comprises a triazole moiety.

In some embodiments, L⁹ is: —C(═O)-L¹⁰-L¹¹-L¹² or —CH₂—C(═O)-L¹⁰-L¹¹-L¹²-; L¹⁰ is absent, —C₁-C₆alkylene, —NR¹³—, substituted or unsubstituted heterocycloalkylene, or substituted or unsubstituted cyclohexylene; L¹¹ is absent, —C₁-C₆ alkylene, substituted or unsubstituted heterocycloalkylene, substituted or unsubstituted cyclohexylene, or —(OCH₂CH₂)ₓ—; L¹² is —NR¹⁴—; R¹³ and R¹⁴ are each independently selected from H or —C₁-C₄ alkyl; and x is 1, 2, 3, 4, 5, or 6.

In some embodiments, L⁹ is: —C(═O)—C₁-C₆ alkylene-NR¹⁴—*, —C(═O)—NR¹³—C₁-C₆ alkylene-NR¹⁴—*, —C(═O)—C₁-C₆ alkylene-(substituted or unsubstituted heterocycloalkylene)-NR¹⁴—*, —C(═O)-(substituted or unsubstituted heterocycloalkylene)-C₁-C₆ alkylene-NR¹⁴—*, —C(═O)—C₁-C₆ alkylene-(substituted or Unsubstituted cyclohexylene)-NR¹⁴—*, —C(═O)-(substituted or unsubstituted cyclohexylene)-C₁-C₆ alkylene-NR¹⁴—*, —C(═O)—C₁-C₆ alkylene-(OCH₂CH₂)ₓ—NR¹⁴—*; —CH₂—C(═O)—C₁-C₆ alkylene-NR¹⁴—*, —CH₂—C(═O)—NR¹³—C₁-C₆ alkylene-NR¹⁴—*, —CH₂—C(═O)—C₁-C₆ alkylene-(substituted or unsubstituted heterocycloalkylene)-NR¹⁴—*, —CH₂—C(═O)-(substituted or unsubstituted heterocycloalkylene)-C₁-C₆ alkylene-NR¹⁴—*, —CH₂—C(═O)—C₁-C₆ alkylene-(substituted or unsubstituted cyclohexylene)-NR¹⁴—*, —CH₂—C(═O)-(substituted or unsubstituted cyclohexylene)-C₁-C₆ alkylene-NR¹⁴—*, or —CH₂—C(═O)—C₁-C₆ alkylene-(OCH₂CH₂)ₓ—NR¹⁴—*; R¹³ and R¹⁴ are each independently selected from H or —C₁-C₄ alkyl; and x is 1, 2, 3, 4, 5, or 6; wherein * denotes the attachment point to R^{b}.

In some embodiments, L⁹ is absent.

In some embodiments, L⁹ is:

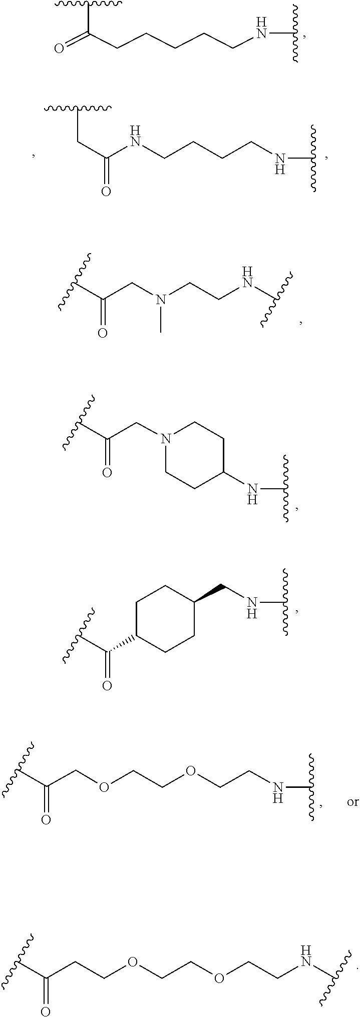

In some embodiments, L⁹ is

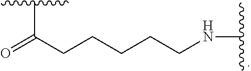 [SMILES: *NCCCCCC(*)=O]

In some embodiments, L⁹ is

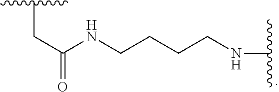 [SMILES: *CC(=O)NCCCCN*]

In some embodiments, L⁹ is

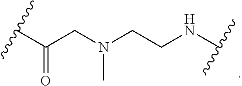 [SMILES: *NCCN(C)CC(*)=O]

In some embodiments, L⁹ is

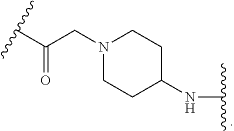 [SMILES: *NC1CCN(CC(*)=O)CC1]

In some embodiments, L⁹ is

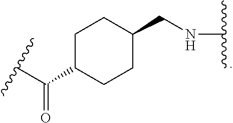

In some embodiments, L⁹ is

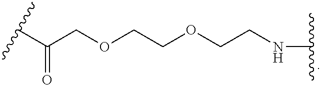 [SMILES: *NCCOCCOCC(*)=O]

In some embodiments, L⁹ is

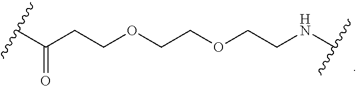 [SMILES: *NCCOCCOCCC(*)=O]

In some embodiments, L⁹ is

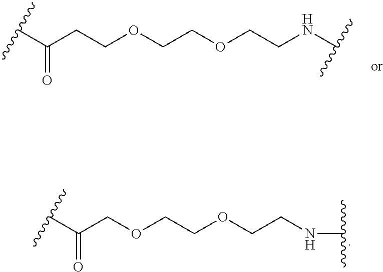 [SMILES: *NCCOCCOCCC(*)=O ; *NCCOCCOCC(*)=O]

In some embodiments, -L⁵-, -L⁶-, or -L⁷- are each independently selected from the group consisting of: —NR¹⁵—C₁-C₆ alkylene-C(═O)—, —NR¹⁵—(CH₂CH₂O)_{y}—C₁-C₆ alkylene-C(═O)—, —NR¹⁵(CH₂CH₂NR¹⁶)_{y}—C₁-C₆alkylene-C(═O)—, —NR¹⁵—C₁-C₆ alkylene-(substituted or unsubstituted heterocycloalkylene)-C(═O)—, —NR¹⁵-(substituted or unsubstituted heterocycloalkylene)-C₁-C₆ alkylene-C(═O)—, —NR¹⁵—C₁-C₆ alkylene-(substituted or unsubstituted cyclohexylene)-C(═O)—, and —NR¹⁵-(substituted or unsubstituted cyclohexylene)-C₁-C₆ alkyl-C(═O)—; R¹⁵ and R¹⁶ are each independently selected from H or —C₁-C₄ alkyl; and y is 1, 2, 3, 4, 5, or 6.

In some embodiments, -L^{S}-, -L⁶-, or -L⁷- are each independently selected from the group consisting of: —NH—C₁-C₆ alkylene-C(═O)—, —NH—(CH₂CH₂O)_{y}—C₁-C₆ alkylene-C(═O)—, —NH(CH₂CH₂NR¹⁶), —C₁-C₆ alkylene-C(═O)—, —NH—C₁-C₆alkylene-(unsubstituted piperidinylene)-C(═O)—, —NH-(unsubstituted piperidinylene)-C₁-C₆ alkylene-C(═O)—, —NH—C₁-C₆ alkylene-(unsubstituted cyclohexylene)-C(═O)—, and —NH-(unsubstituted cyclohexylene)-C₁-C₆ alkylene-C(═O)—.

In some embodiments, -L⁵- is —NR¹⁵—C₁-C₆ alkylene-C(═O)—, —NR¹⁵—(CH₂CH₂O)_{y}—C₁-C₆ alkylene-C(═O)—, or —NR¹⁵(CH₂CH₂NR¹⁶)_{y}—C₁-C₆ alkylene-C(═O)—, R¹⁵ and R¹⁶ are each independently selected from H or —CH₃; and y is 2, 3, or 4.

In some embodiments, -L⁶- is —NR¹⁵—C₁-C₆ alkylene-C(═O)—, —NR¹⁵—(CH₂CH₂O)_{y}—C₁-C₆ alkylene-C(═O)—, or —NR¹⁵(CH₂CH₂NR¹⁶)_{y}—C₁-C₆ alkylene-C(═O)—, R¹⁵ and R¹⁶ are each independently selected from H or —CH₃; and y is 2, 3, or 4.

In some embodiments, -L⁷- is —NR¹⁵—C₁-C₆ alkylene-C(═O)—, —NR¹⁵—(CH₂CH₂O)_{y}—C₁-C₆ alkylene-C(═O)—, or —NR¹⁵(CH₂CH₂NR¹⁶)_{y}—C₁-C₆ alkylene-C(═O)—, R¹⁵ and R¹⁶ are each independently selected from H or —CH₃; and y is 2, 3, or 4.

In some embodiments, -L⁵-, -L⁶-, or -L⁷- are each independently selected from the group consisting of

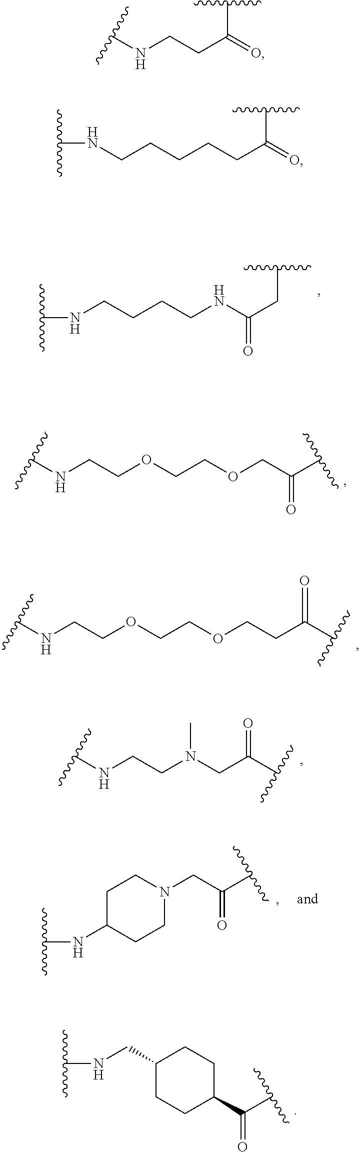

In some embodiments, -L⁵- is

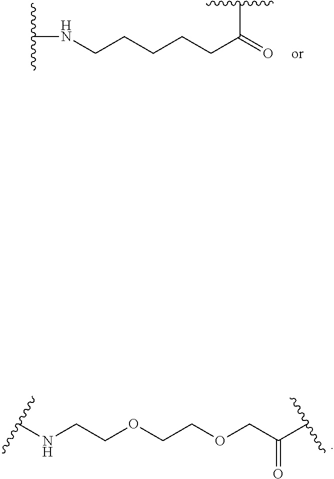 [SMILES: *NCCCCCC(*)=O ; *NCCOCCOCC(*)=O]

In some embodiments, -L⁵- is

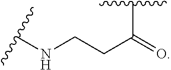 [SMILES: *NCCC(*)=O]

In some embodiments, -L⁵- is

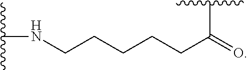 [SMILES: *NCCCCCC(*)=O]

In some embodiments, -L⁵- is

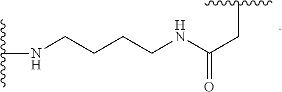 [SMILES: *CC(=O)NCCCCN*]

In some Embodiments, -L⁵- is

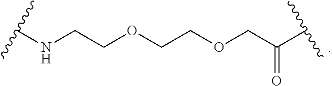 [SMILES: *NCCOCCOCC(*)=O]

In some embodiments, -L⁵- is

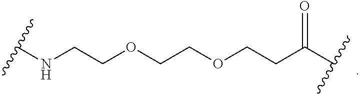 [SMILES: *NCCOCCOCCC(*)=O]

In some embodiments, -L⁵- is

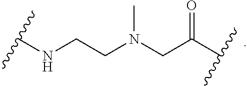 [SMILES: *NCCN(C)CC(*)=O]

In some embodiments, -L⁵- is

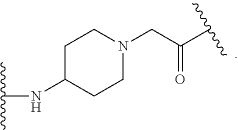 [SMILES: *NC1CCN(CC(*)=O)CC1]

In some embodiments, -L⁵- is

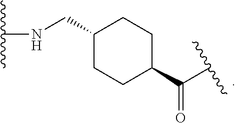 [SMILES: *NC[C@H]1CC[C@H](C(*)=O)CC1]

In some embodiments, -L⁵- is substituted or unsubstituted piperazine. In some embodiments, -L⁵- is

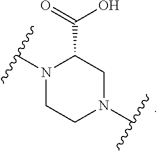 [SMILES: *N1CCN(*)[C@H](C(=O)O)C1]

In some embodiments, -L⁶- is

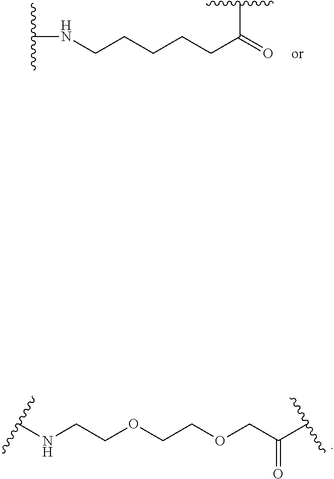 [SMILES: *NCCCCCC(*)=O ; *NCCOCCOCC(*)=O]

In some embodiments, -L⁶- is

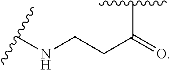 [SMILES: *NCCC(*)=O]

In some embodiments, -L⁶- is

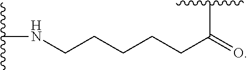 [SMILES: *NCCCCCC(*)=O]

In some embodiments, -L⁶- is

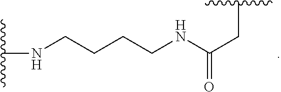 [SMILES: *CC(=O)NCCCCN*]

In some embodiments, -L⁶- is

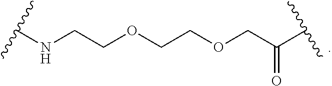 [SMILES: *NCCOCCOCC(*)=O]

In some embodiments, -L⁶- is

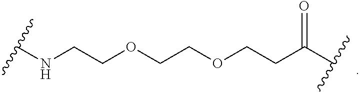 [SMILES: *NCCOCCOCCC(*)=O]

In some embodiments, -L⁶- is

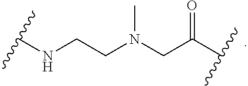 [SMILES: *NCCN(C)CC(*)=O]

In some embodiments, -L⁶- is

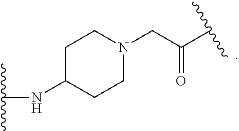 [SMILES: *NC1CCN(CC(*)=O)CC1]

In some embodiments, -L⁶- is

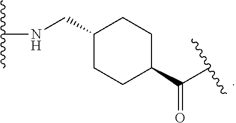 [SMILES: *NC[C@H]1CC[C@H](C(*)=O)CC1]

In some embodiments, -L⁷- is

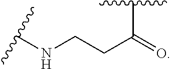 [SMILES: *NCCC(*)=O]

In some embodiments, -L⁷- is

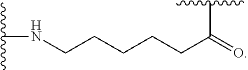 [SMILES: *NCCCCCC(*)=O]

in some embodiments, -L⁷- is

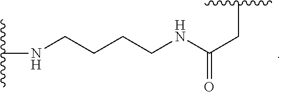 [SMILES: *CC(=O)NCCCCN*]

In some embodiments, -L⁷- is

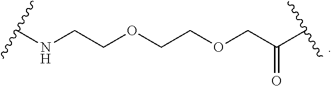 [SMILES: *NCCOCCOCC(*)=O]

In some embodiments, -L⁷- is

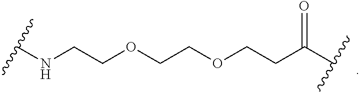 [SMILES: *NCCOCCOCCC(*)=O]

In some embodiments, -L⁷- is

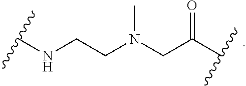 [SMILES: *NCCN(C)CC(*)=O]

In some embodiments, -L⁷- is

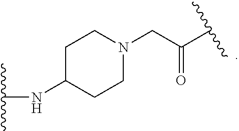 [SMILES: *NC1CCN(CC(*)=O)CC1]

In some embodiments, -L⁷- is

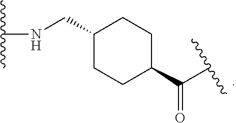 [SMILES: *NC[C@H]1CC[C@H](C(*)=O)CC1]

In some embodiments, L⁹ is —C(═O)-L¹⁰-L¹¹-L¹². In some embodiments, L⁹ is —CH₂—C(═O)-L¹⁰-L¹¹-L¹².

In some embodiments, L¹⁰ is absent. In some embodiments, L¹⁰ is —C₁-C₆ alkylene. In some embodiments, L¹¹ is —CH₂—. In some embodiments, L¹⁰ is —CH₂CH₂—. In some embodiments. L¹⁰ is —CH₂CH₂CH₂CH₂CH₂—. In some embodiments, L¹⁰ is —NH—. In some embodiments, L¹⁰ is —NCH₃—. In some embodiments, L¹⁰ is substituted or unsubstituted heterocycloalkylene. In some embodiments, L¹⁰ is substituted or unsubstituted cyclohexylene.

In some embodiments, L¹¹ is absent. In some embodiments, L¹¹ is —C₁-C₆ alkylene. In some embodiments, L¹¹ is substituted or unsubstituted heterocycloalkylene. In some embodiments, L¹¹ is substituted or unsubstituted cyclohexylene. In some embodiments, L¹¹ is —(OCH₂CH₂)ₓ—. In some embodiments, L¹¹ is —(OCH₂CH₂)₂—.

In some embodiments, L¹² is —NH—. In some embodiments, L¹² is —N(CH₃)—.

In some embodiments, R¹³ is H. In some embodiments, R¹³ is —CH₃. In some embodiments, R¹⁴ is H. In some embodiments, R¹⁴ is —CH₃.

In some embodiments, x is 1. In some embodiments, x is 2. In some embodiments, x is 3.

In some embodiments, x is 4. In some embodiments, x is 5. In some embodiments, x is 6.

In some embodiments, y is 1. In some embodiments, y is 2. In some embodiments, y is 3.

In some embodiments, y is 4. In some embodiments, y is 5. In some embodiments, y is 6.

### Representative Linker and Chelating Moieties

In some embodiments, R^{b}-L⁹- is

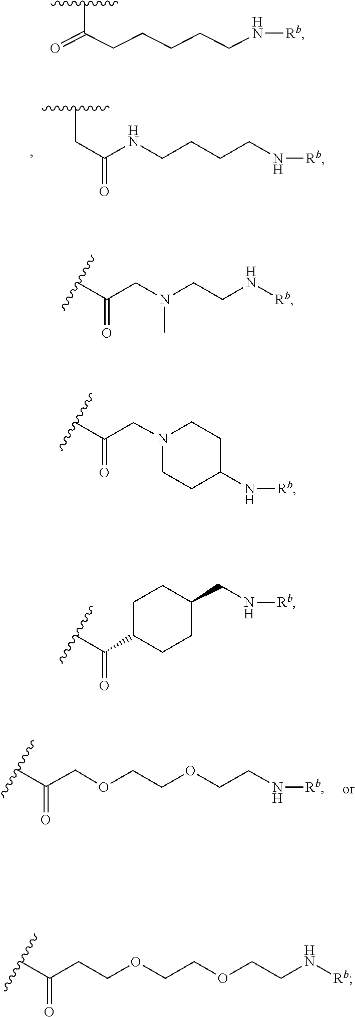

wherein R^{b} is.

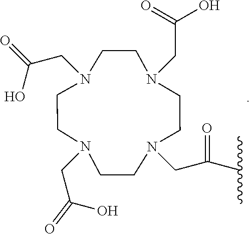 [SMILES: *C(=O)CN1CCN(CC(=O)O)CCN(CC(=O)O)CCN(CC(=O)O)CC1]

In some embodiments, R^{b}-L⁹- is

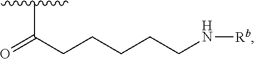

wherein R^{b} is

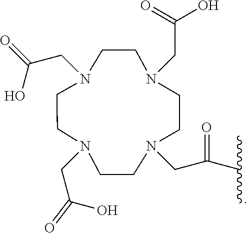 [SMILES: *C(=O)CN1CCN(CC(=O)O)CCN(CC(=O)O)CCN(CC(=O)O)CC1]

In some embodiments, R^{b}-L⁹- is

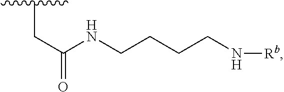

wherein R^{b} is

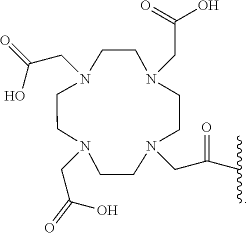 [SMILES: *C(=O)CN1CCN(CC(=O)O)CCN(CC(=O)O)CCN(CC(=O)O)CC1]

In some embodiments, R^{b}-L⁹- is

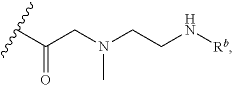

wherein R^{b} is

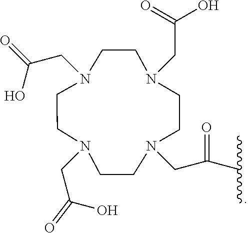 [SMILES: *C(=O)CN1CCN(CC(=O)O)CCN(CC(=O)O)CCN(CC(=O)O)CC1]

In some embodiments, R^{b}-L⁹- is

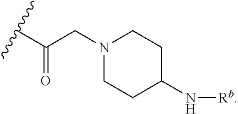

wherein R^{b} is

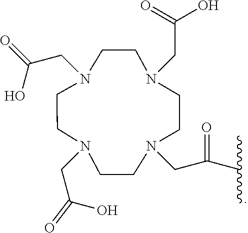 [SMILES: *C(=O)CN1CCN(CC(=O)O)CCN(CC(=O)O)CCN(CC(=O)O)CC1]

In some embodiments, R^{b}-L⁹- is

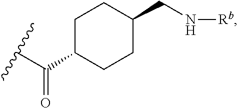

wherein R^{b} is

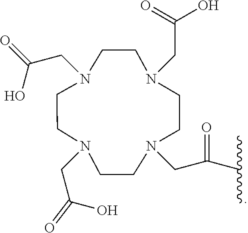 [SMILES: *C(=O)CN1CCN(CC(=O)O)CCN(CC(=O)O)CCN(CC(=O)O)CC1]

In some embodiments, R^{b}-L⁹- is

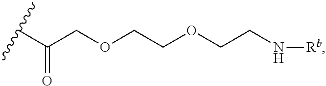

wherein R^{b} is

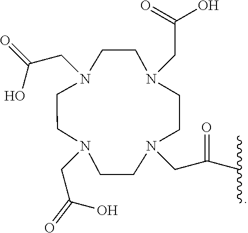 [SMILES: *C(=O)CN1CCN(CC(=O)O)CCN(CC(=O)O)CCN(CC(=O)O)CC1]

In some embodiments, R^{b}-L⁹- is

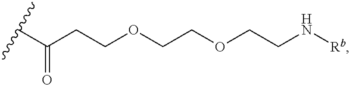

wherein R^{b} is

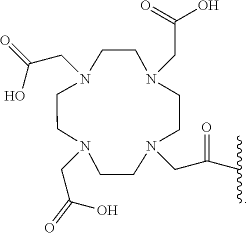 [SMILES: *C(=O)CN1CCN(CC(=O)O)CCN(CC(=O)O)CCN(CC(=O)O)CC1]

In some embodiments, L⁹ is absent and R^{b} is directly attached to a nitrogen atom of the amino acid side chain of X⁴, X⁵, X⁶ or X⁷. In some embodiments, L⁹ is absent and R is directly attached to the nitrogen atom of the Lys side chain of X⁴, X⁵, X⁶ or X⁷, wherein X⁴, X⁵, X⁶ or X⁷ is lysine. In some embodiments, L⁹ is absent and R^{b} is directly attached to the nitrogen atom of the Lys side chain of X⁵, wherein X⁵ is lysine. In some embodiments, L⁹ is absent and R^{b} is directly attached to the nitrogen atom of the Lys side chain of X⁶, wherein X⁶ is lysine. In some embodiments, L⁹ is absent and R^{b} is directly attached to the nitrogen atom of the Lys side chain of X⁷, wherein X⁷ is lysine.

### Representative Compounds

In some embodiments, the compound of Formula (I) is any one of compounds 219 to 543, or a pharmaceutically acceptable salt thereof, or a radionuclide complex thereof.

In some embodiments, the compound of Formula (1) or a pharmaceutically acceptable salt thereof, has the following chemical structure:

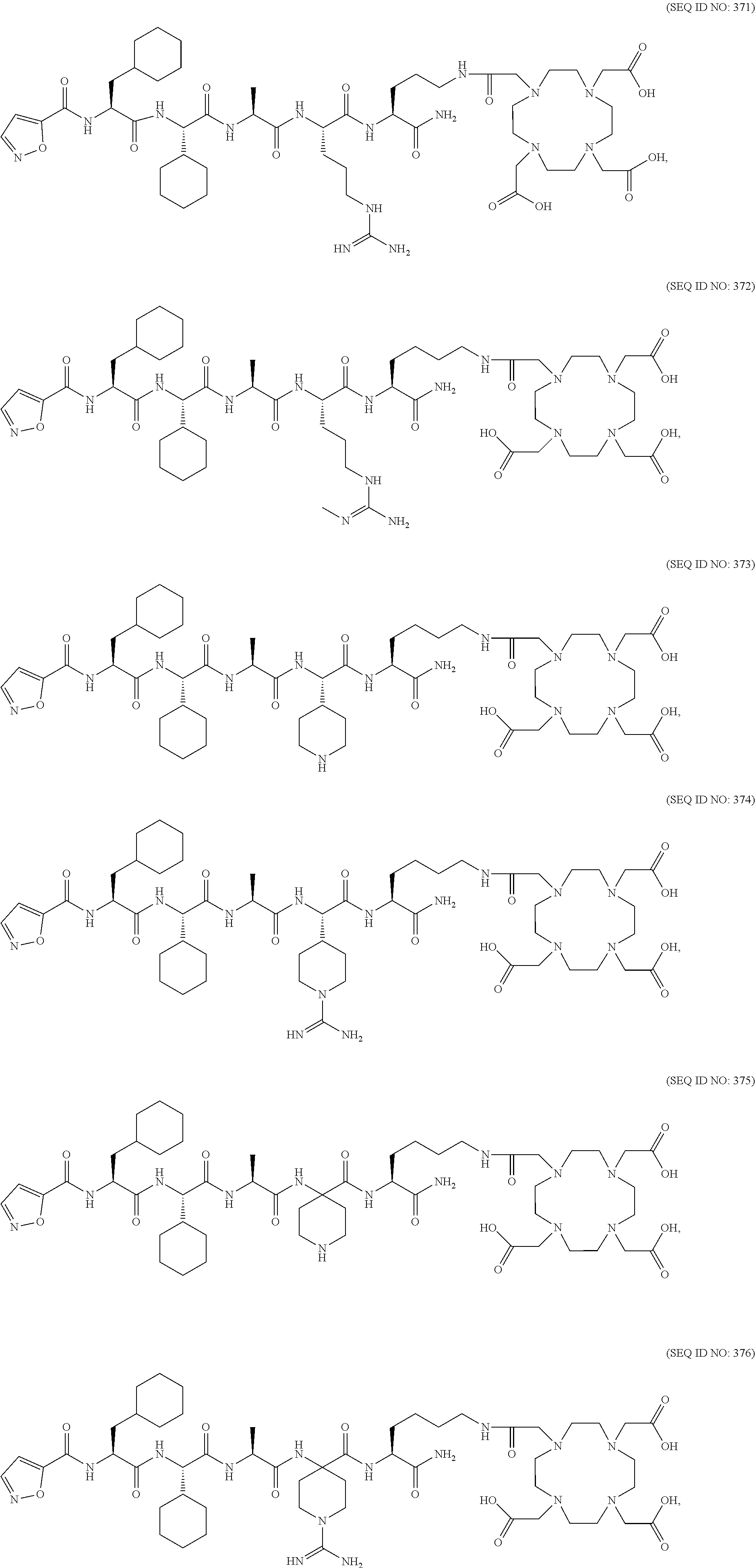

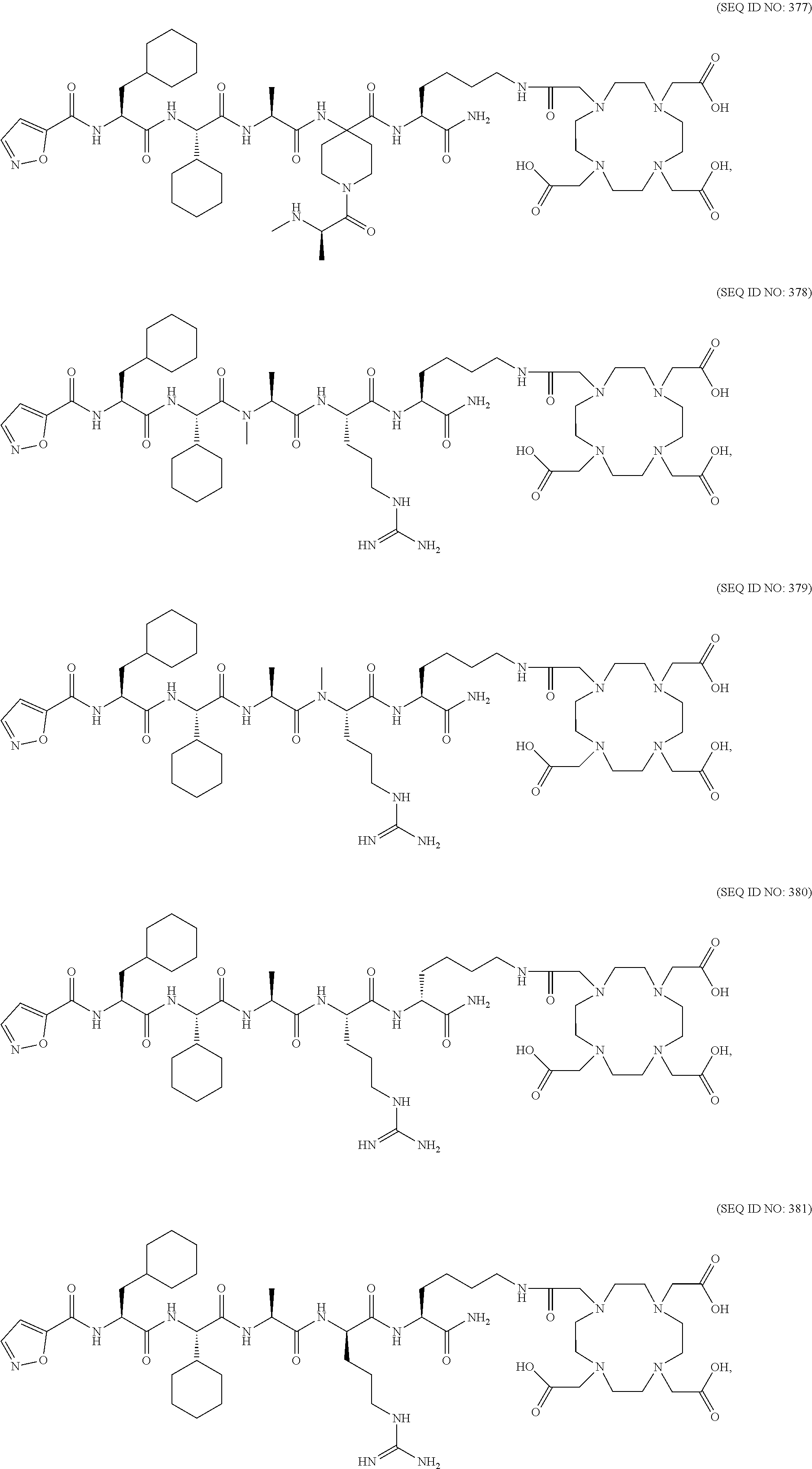

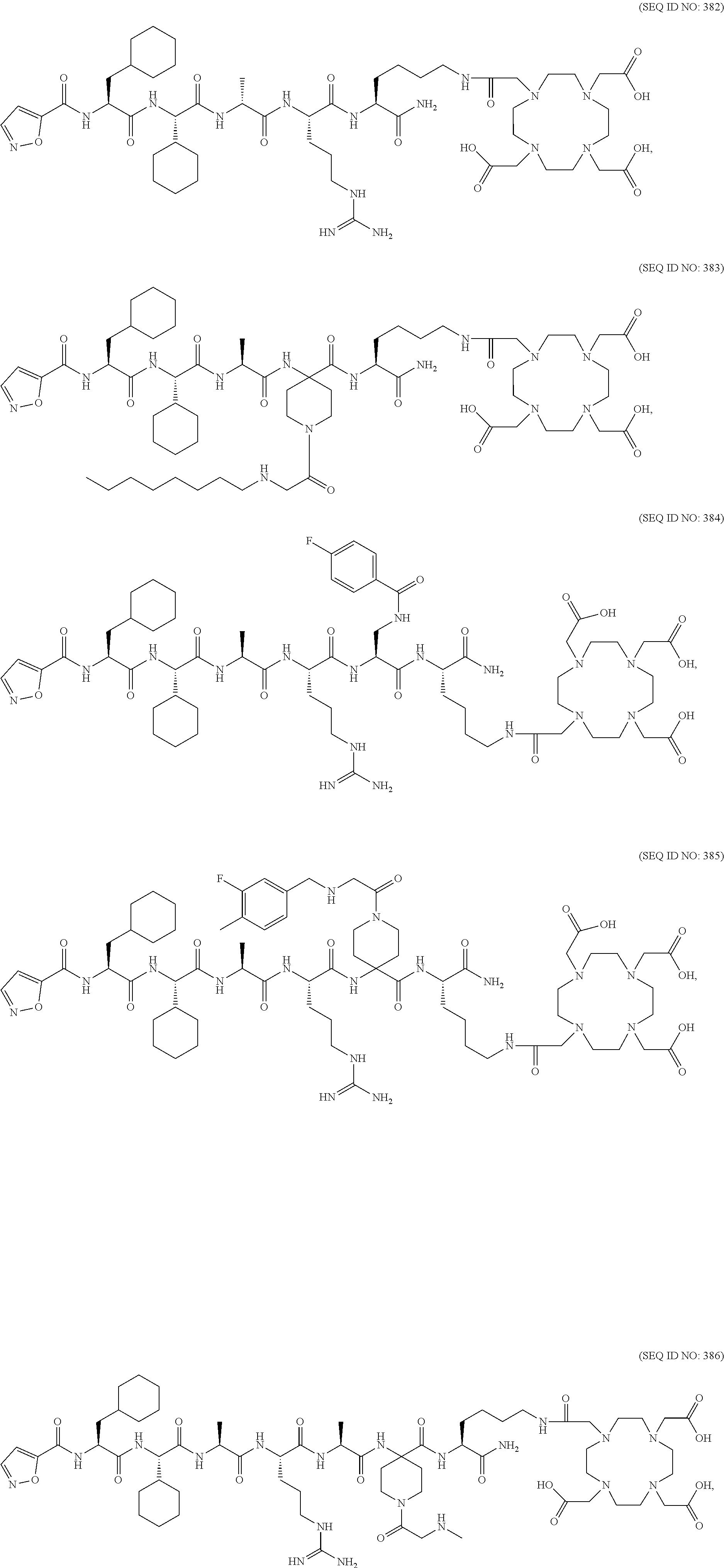

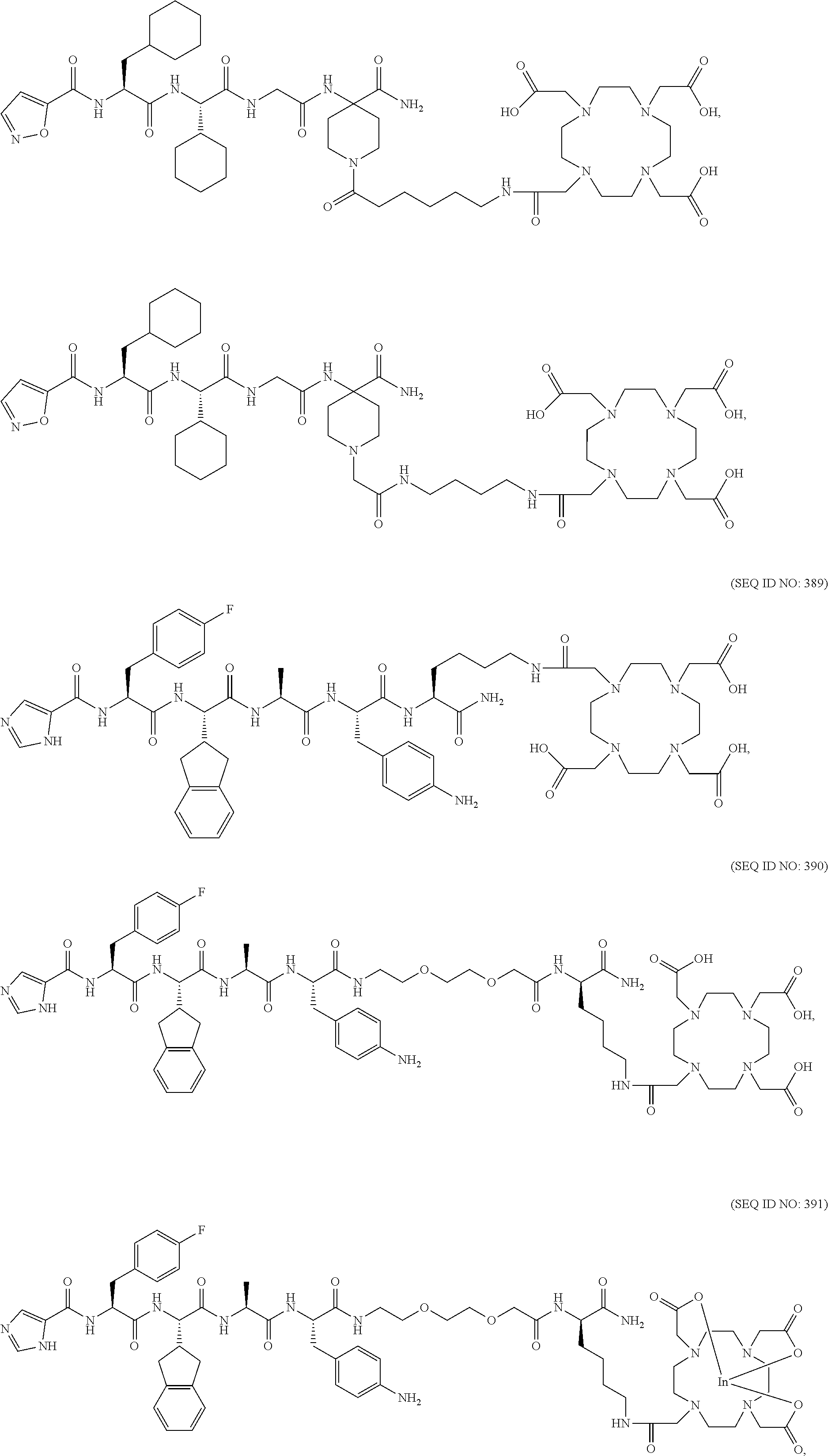

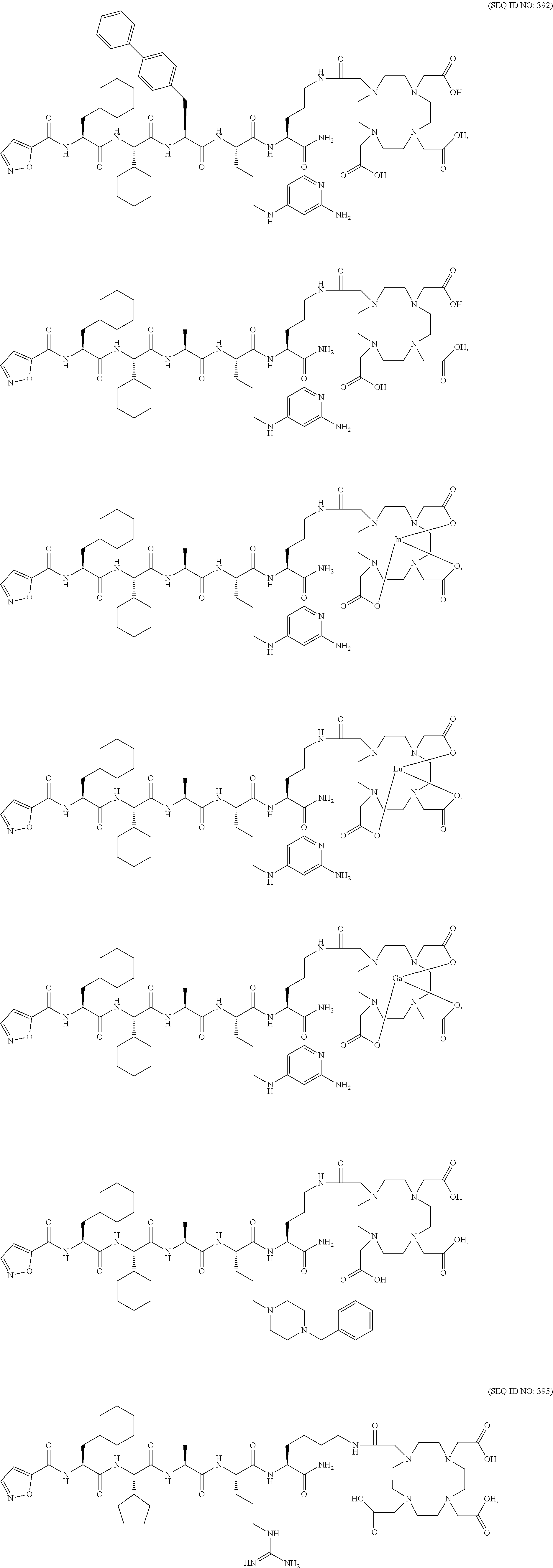

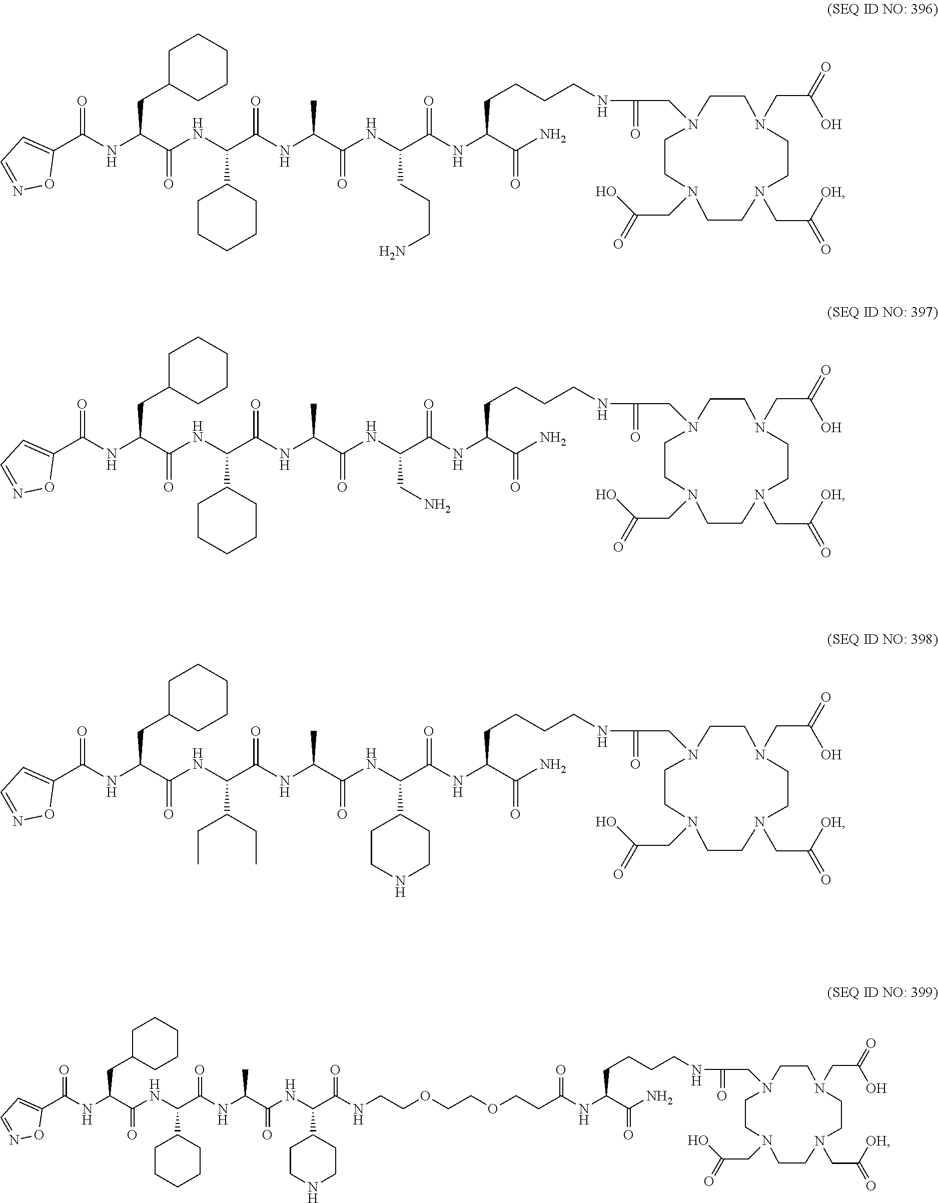

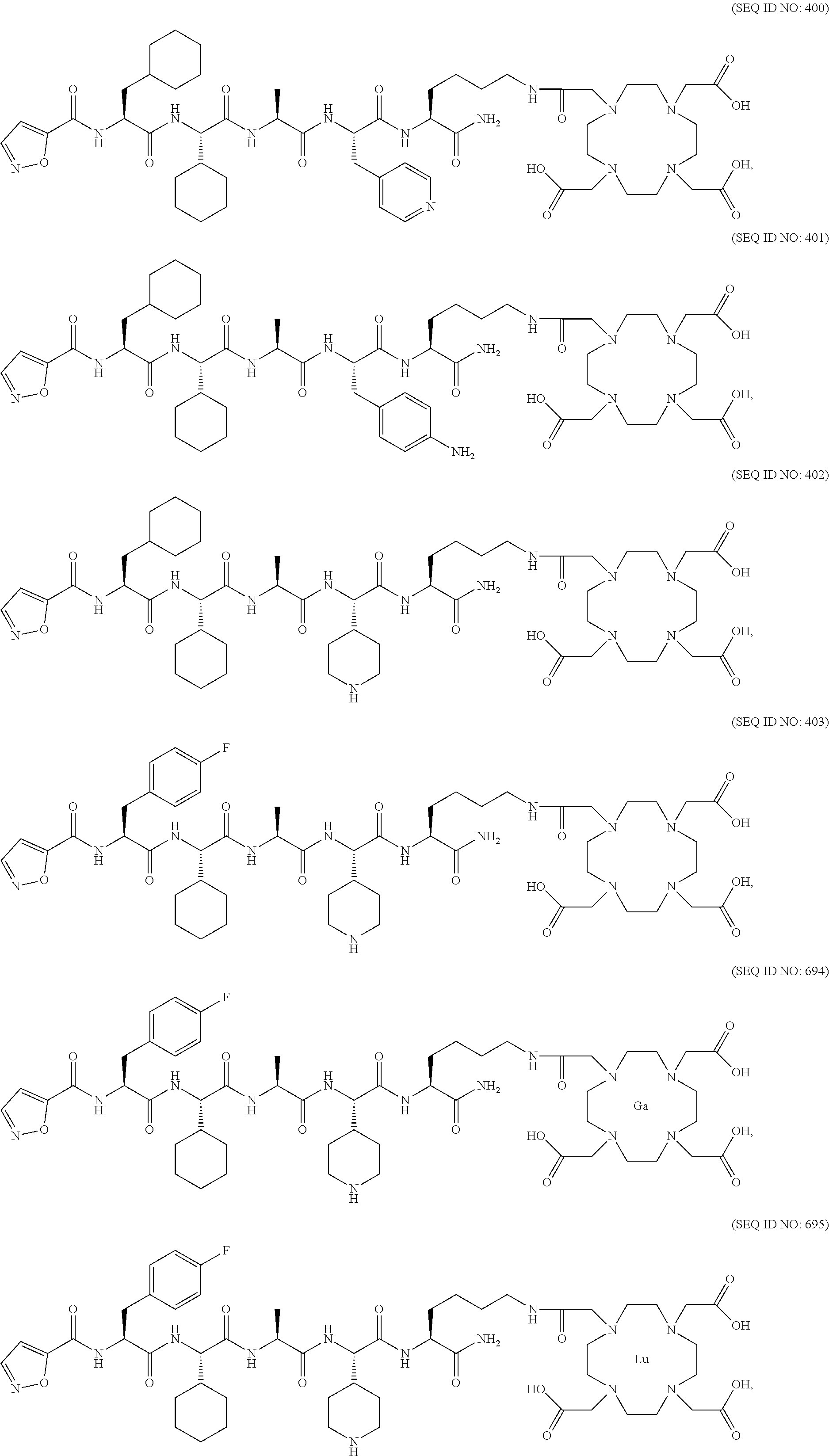

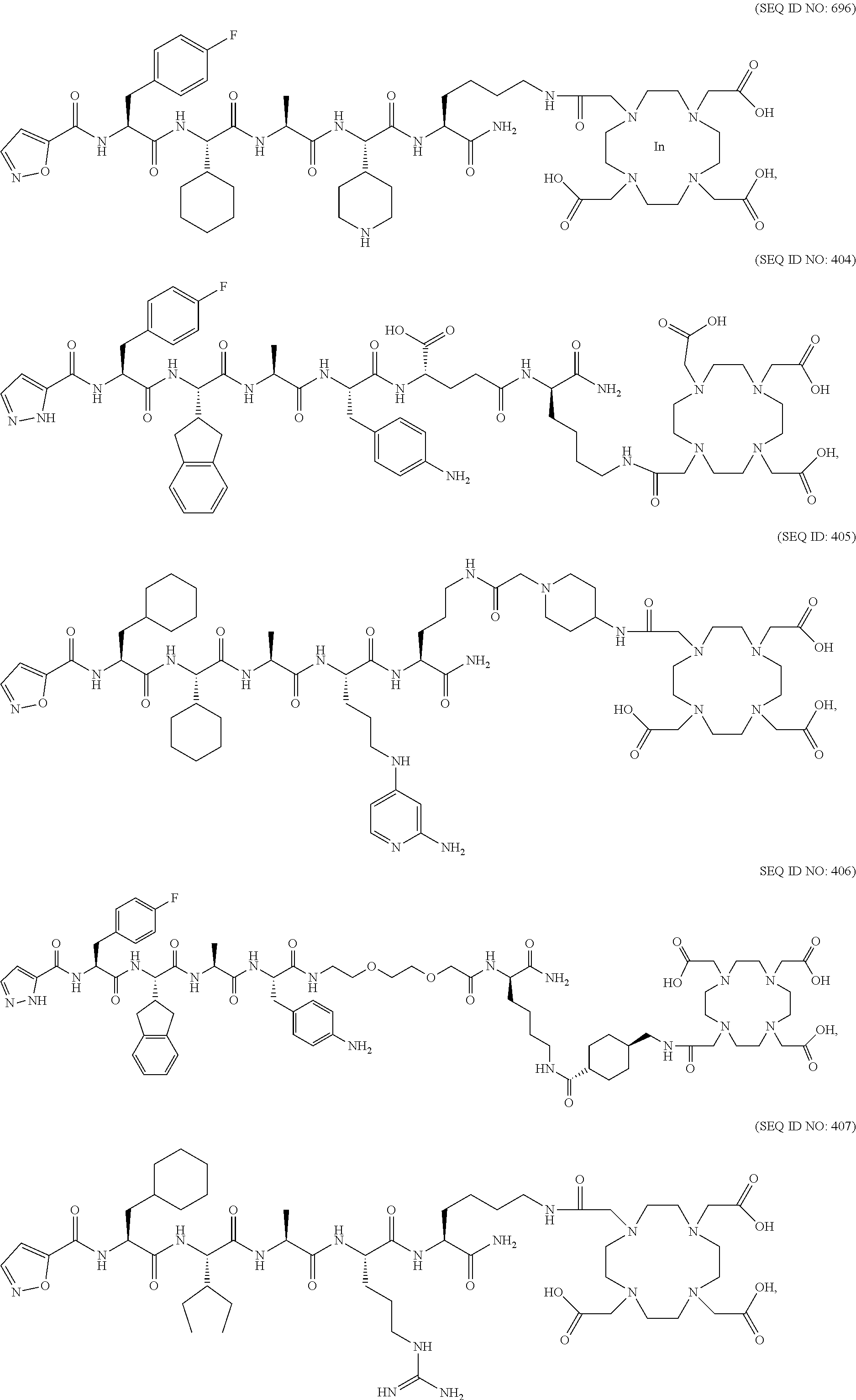

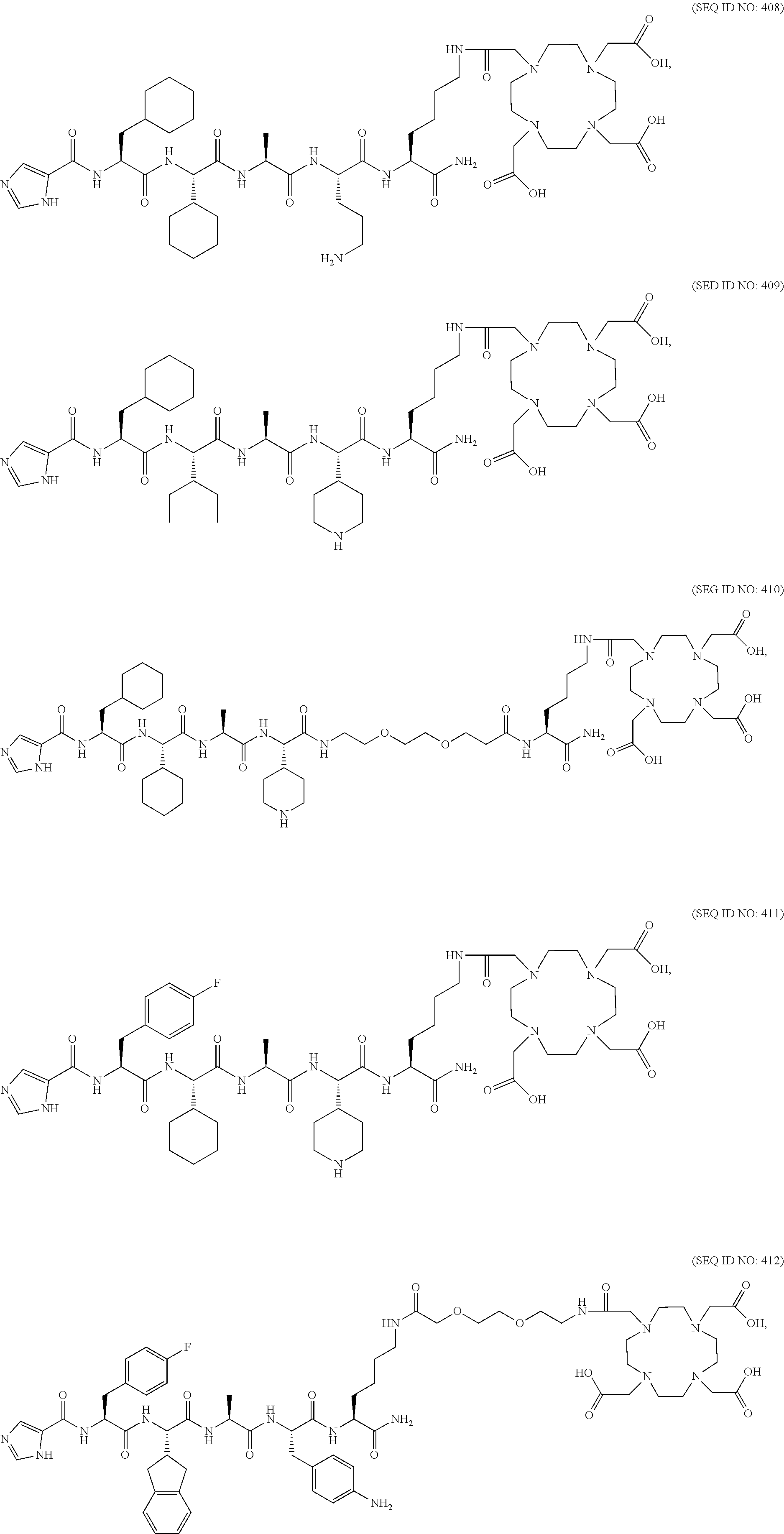

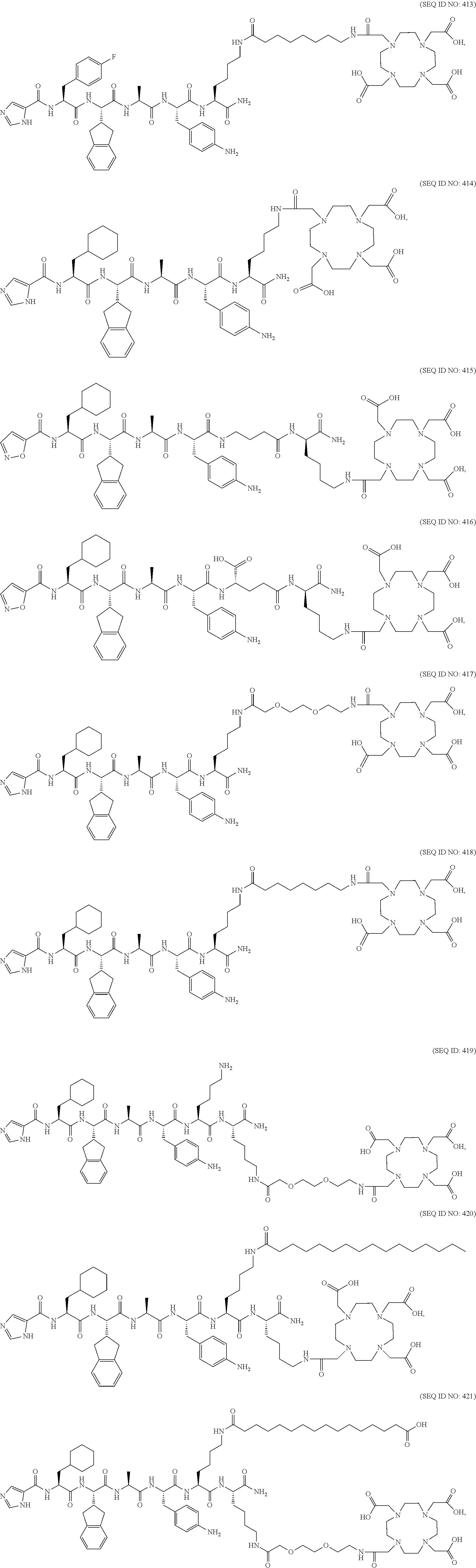

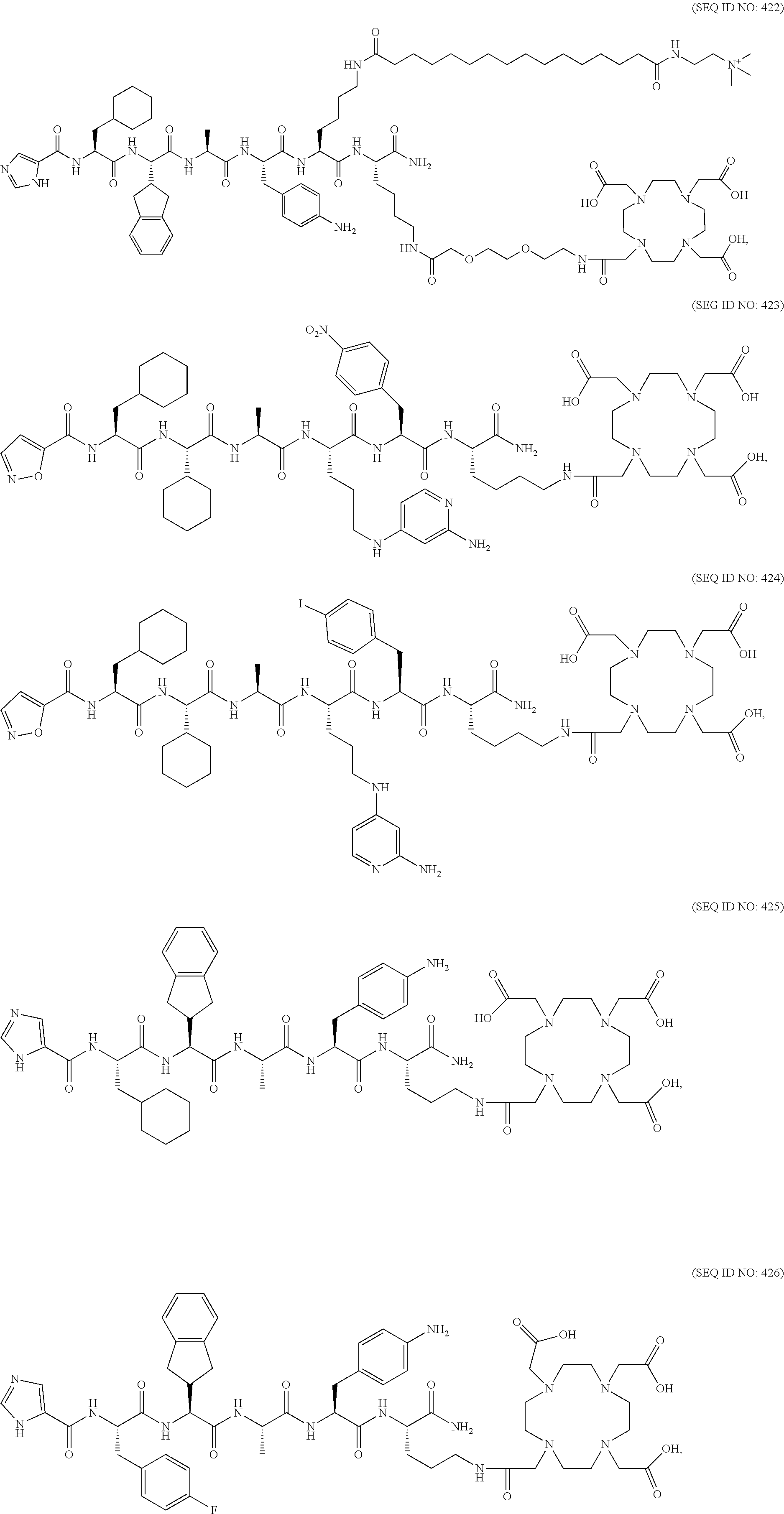

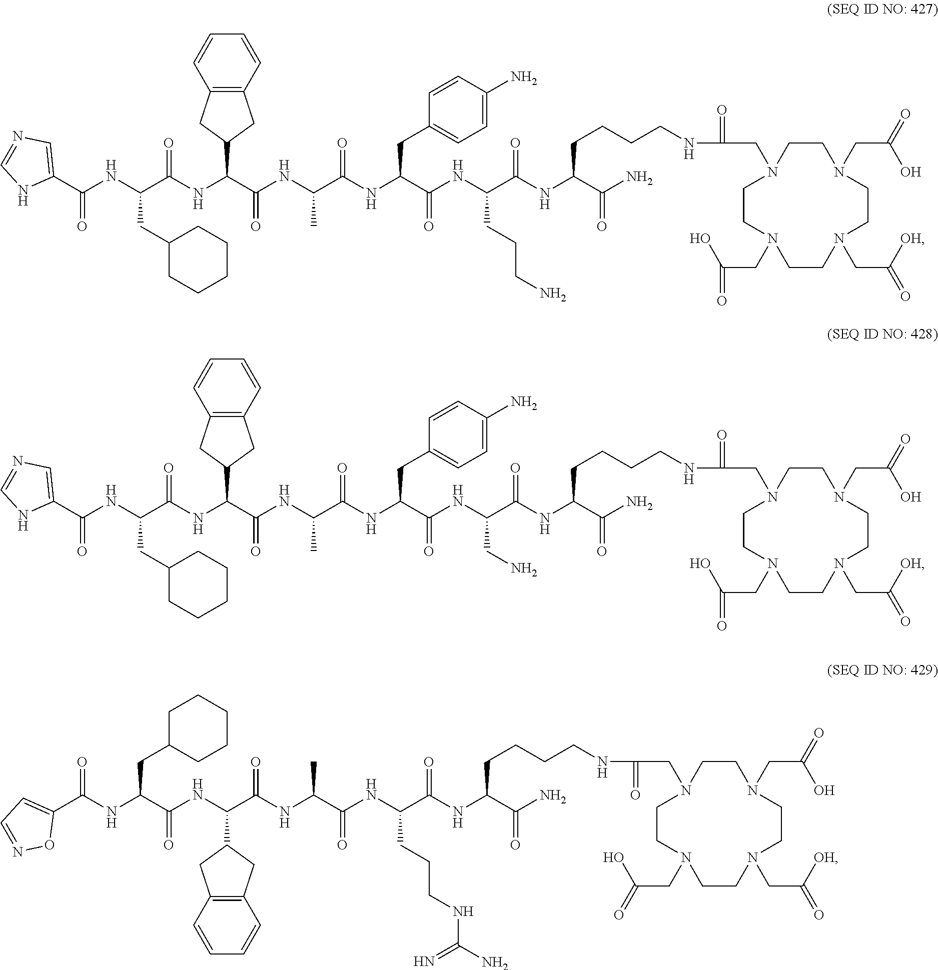

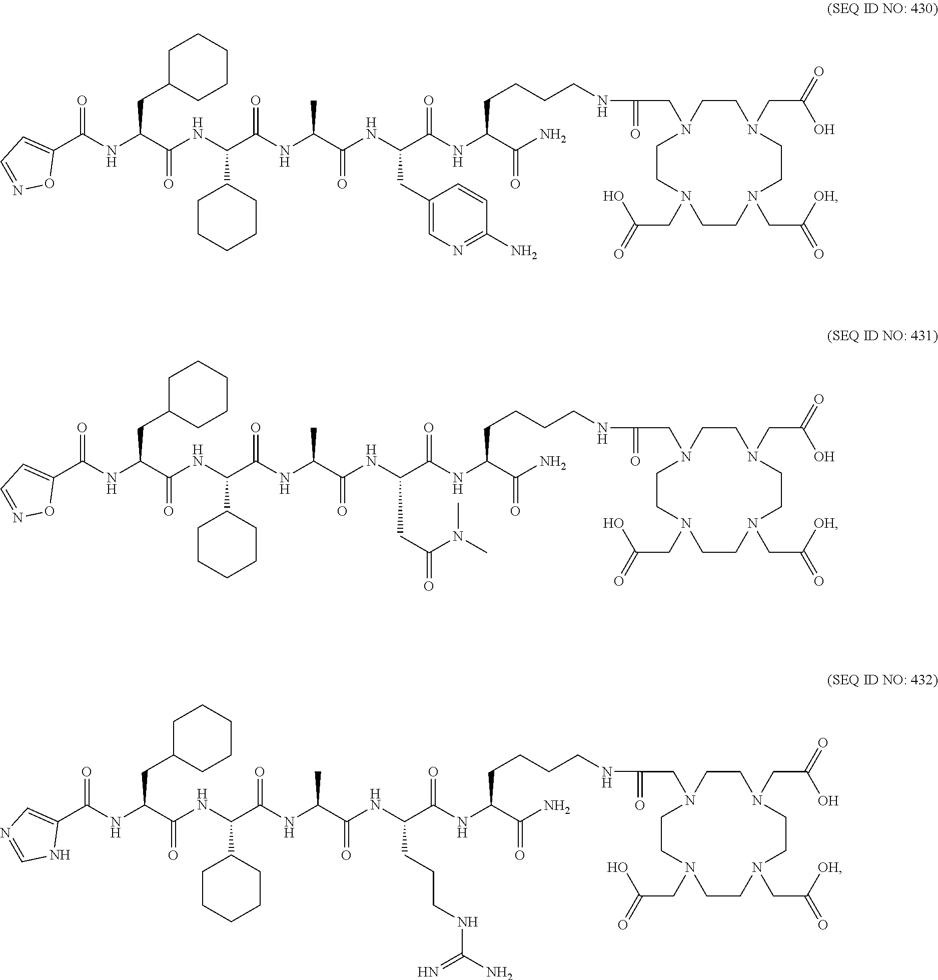 [SMILES: C[C@H](NC(=O)[C@@H](NC(=O)[C@H](CC1CCCCC1)NC(=O)c1ccno1)C1CCCCC1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(N)=O ; C[C@H](NC(=O)[C@@H](NC(=O)[C@H](CC1CCCCC1)NC(=O)c1ccno1)C1CCCCC1)C(=O)N[C@@H](CC(=O)N(C)C)C(=O)N[C@@H](CCCCNC(=O)CN1CCN(CC(=O)O)CCN(CC(=O)O)CCN(CC(=O)O)CC1)C(N)=O ; C[C@H](NC(=O)[C@@H](NC(=O)[C@H](CC1CCCCC1)NC(=O)c1cnc[nH]1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

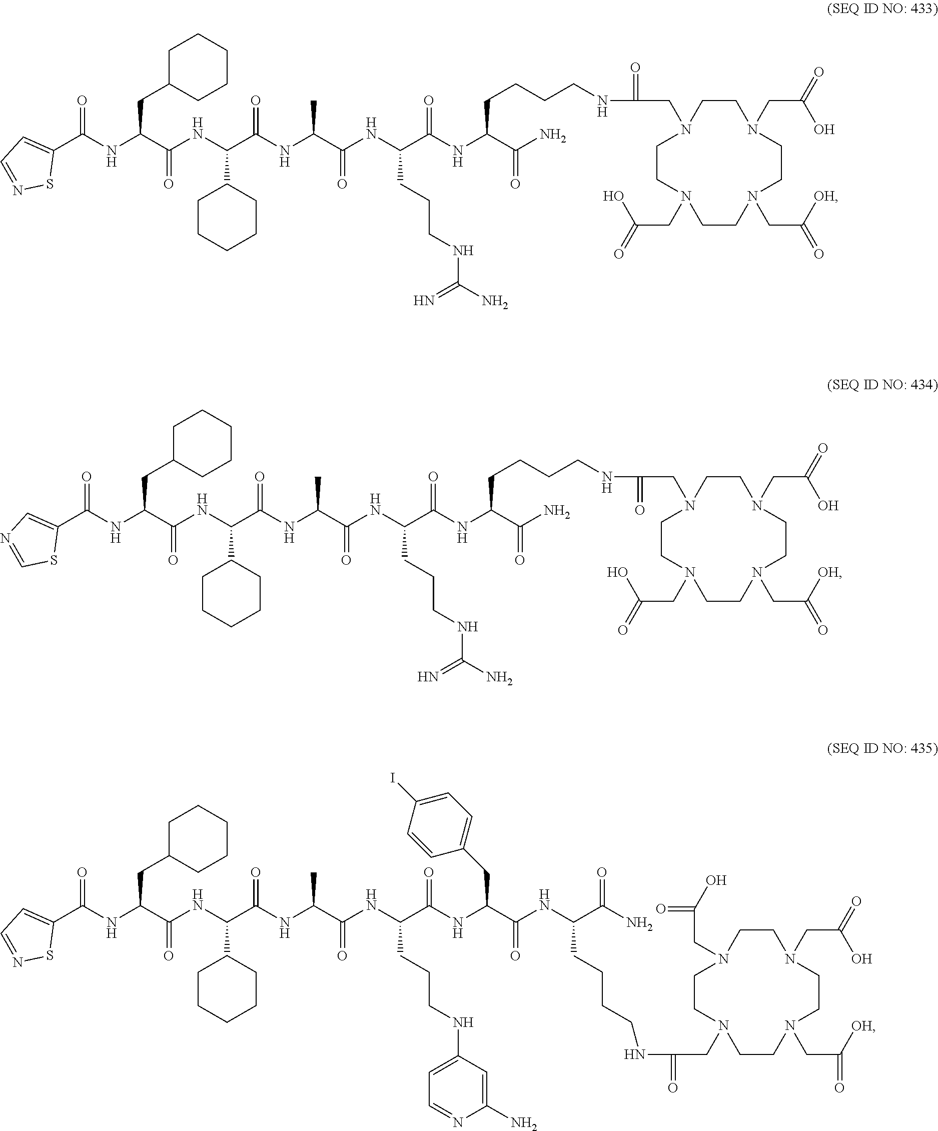

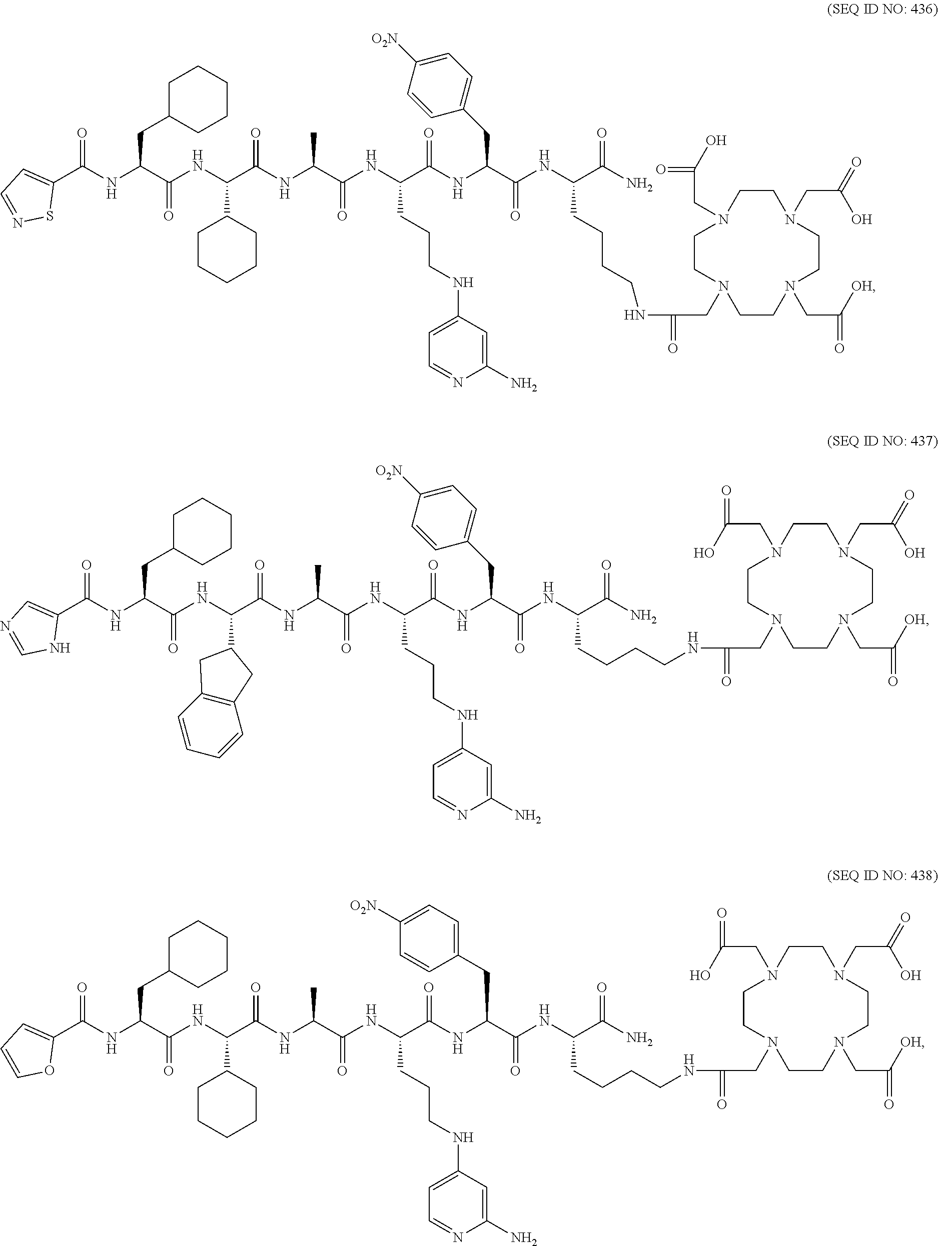

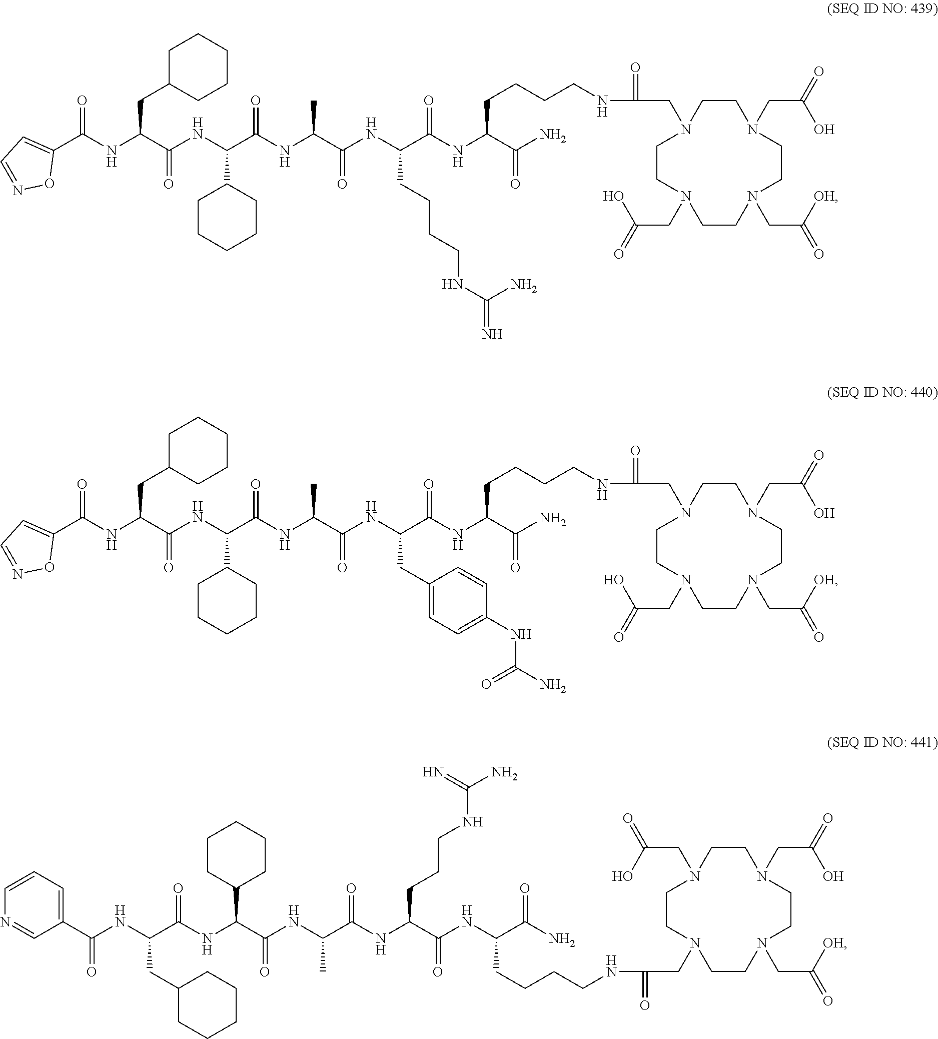 [SMILES: C[C@H](NC(=O)[C@@H](NC(=O)[C@H](CC1CCCCC1)NC(=O)c1ccno1)C1CCCCC1)C(=O)N[C@@H](CCCCNC(=N)N)C(=O)N[C@@H](CCCCNC(=O)CN1CCN(CC(=O)O)CCN(CC(=O)O)CCN(CC(=O)O)CC1)C(N)=O ; C[C@H](NC(=O)[C@@H](NC(=O)[C@H](CC1CCCCC1)NC(=O)c1ccno1)C1CCCCC1)C(=O)N[C@@H](Cc1ccc(NC(N)=O)cc1)C(=O)N[C@@H](CCCCNC(=O)CN1CCN(CC(=O)O)CCN(CC(=O)O)CCN(CC(=O)O)CC1)C(N)=O ; C[C@H](NC(=O)[C@@H](NC(=O)[C@H](CC1CCCCC1)NC(=O)c1cccnc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

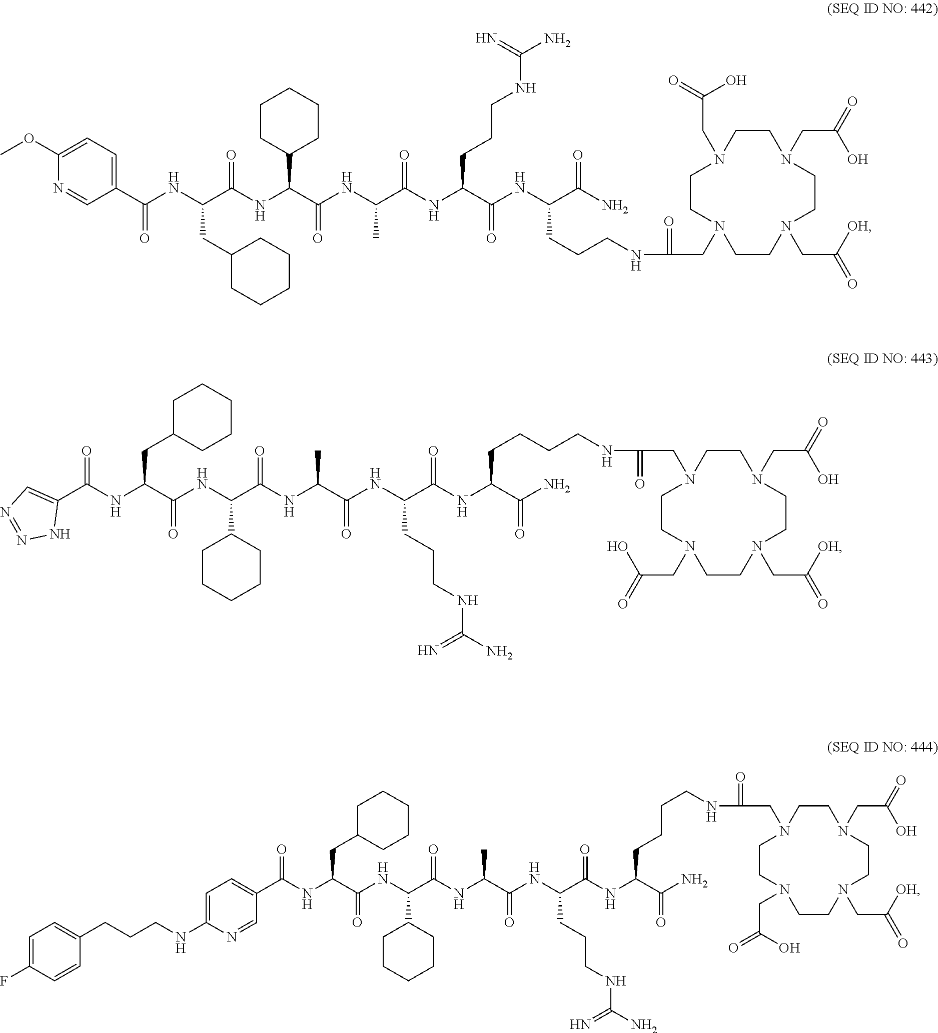

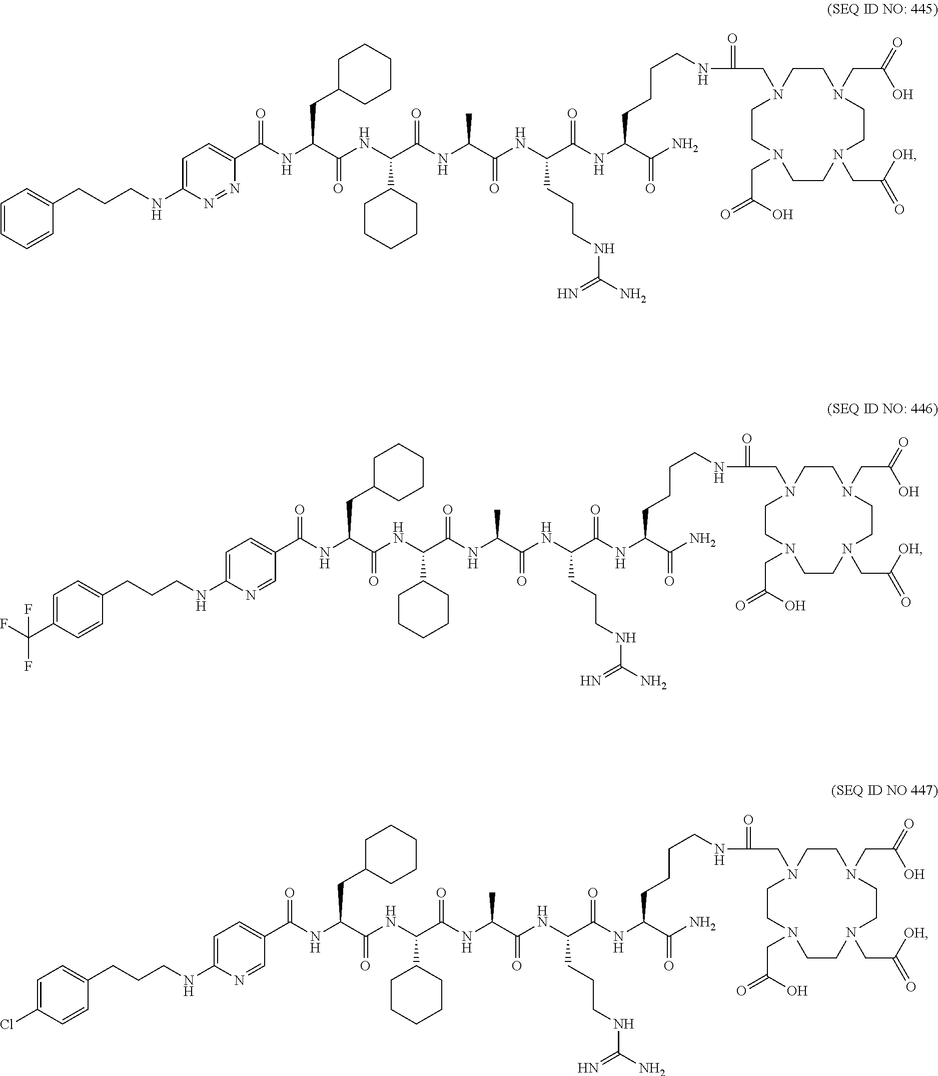

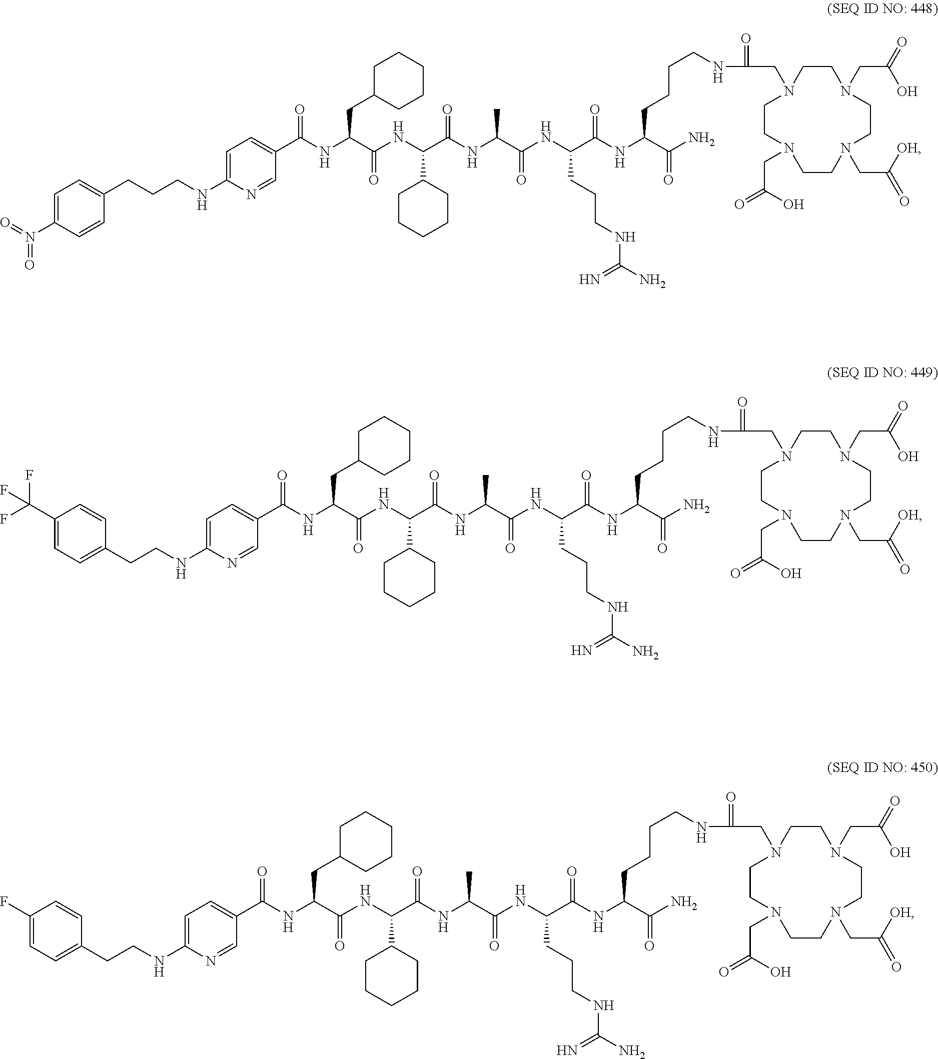

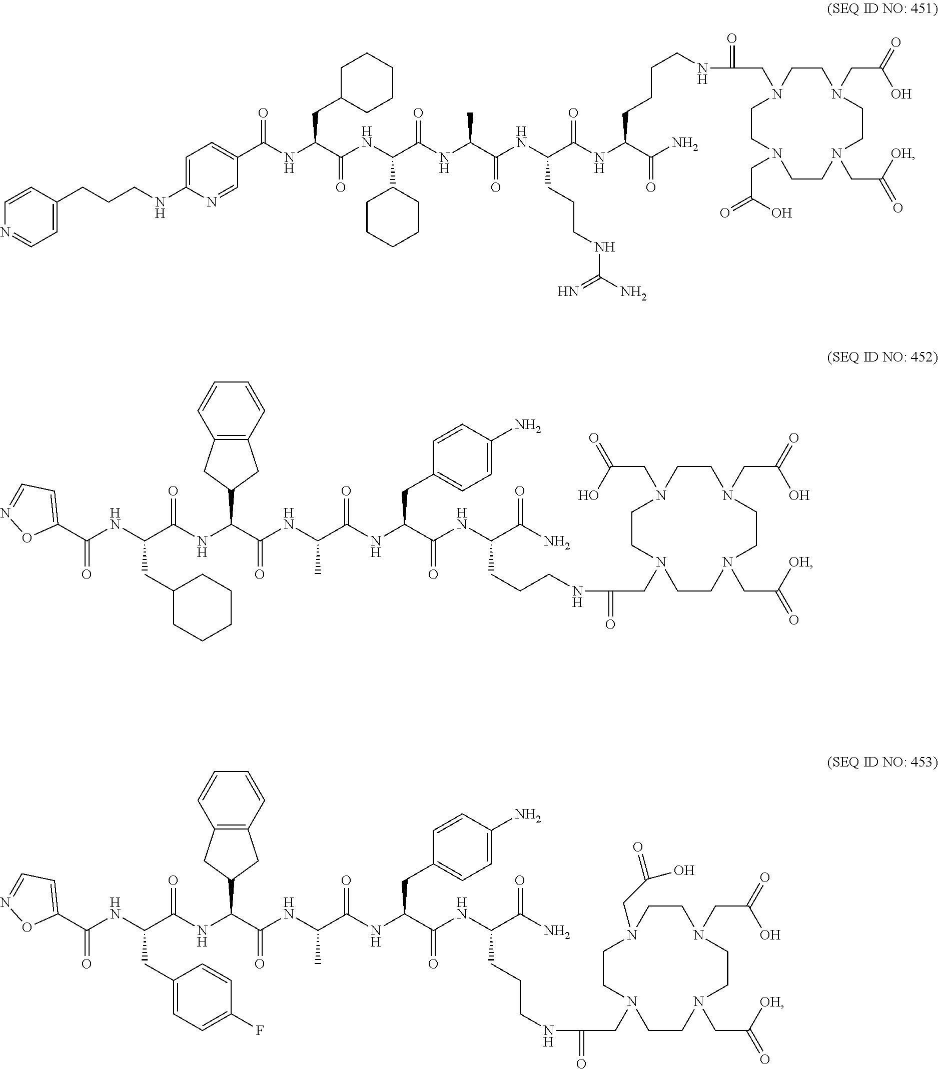

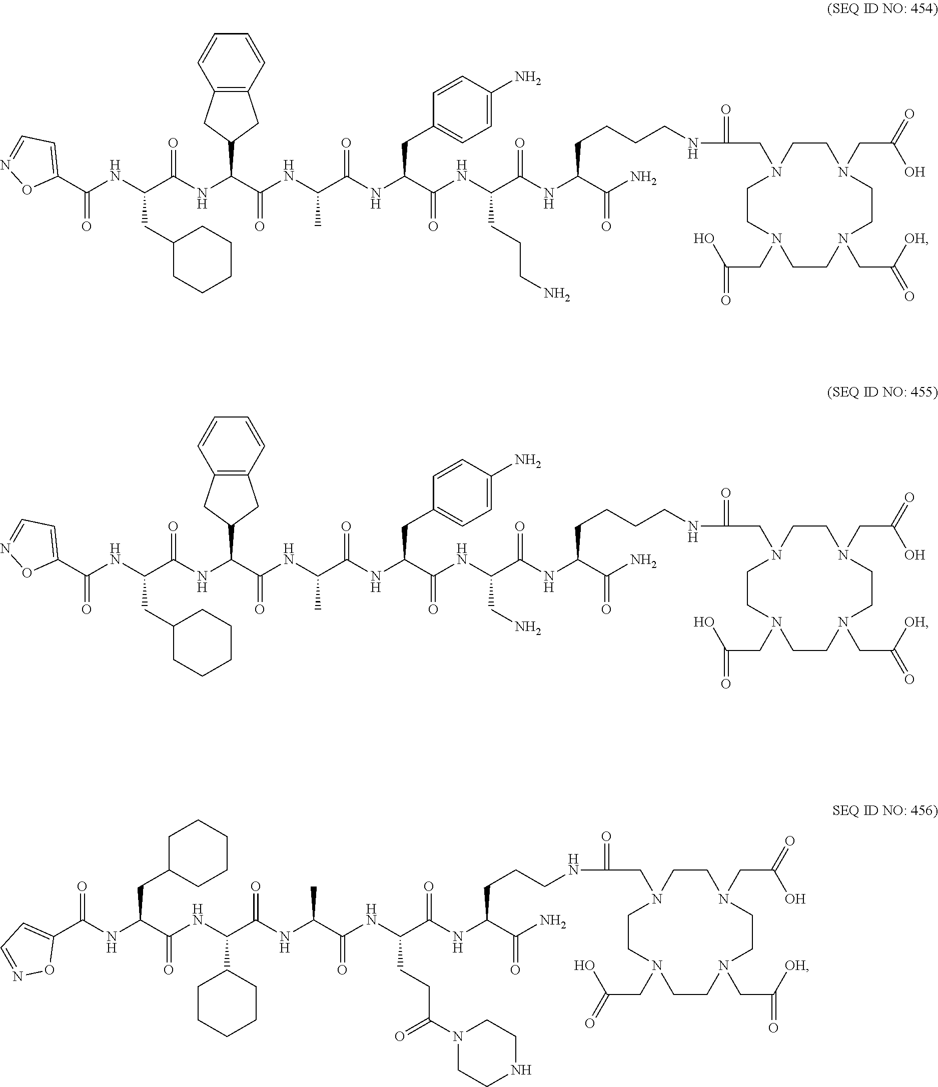

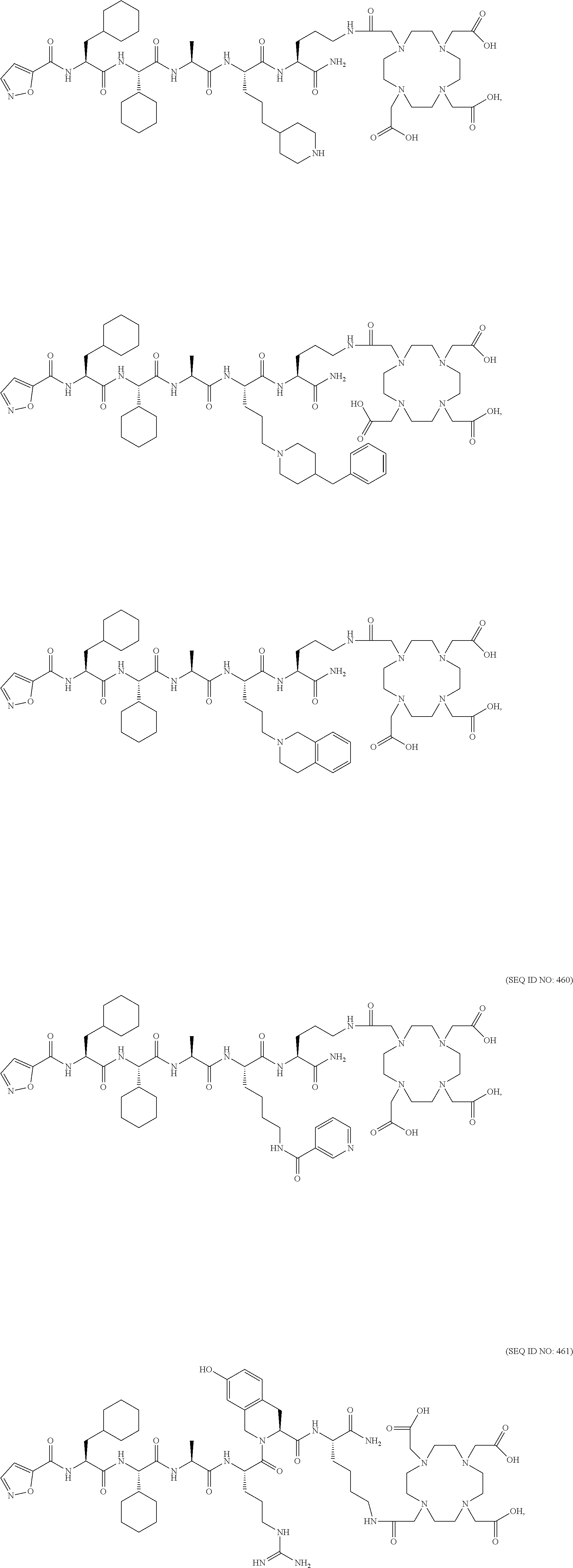

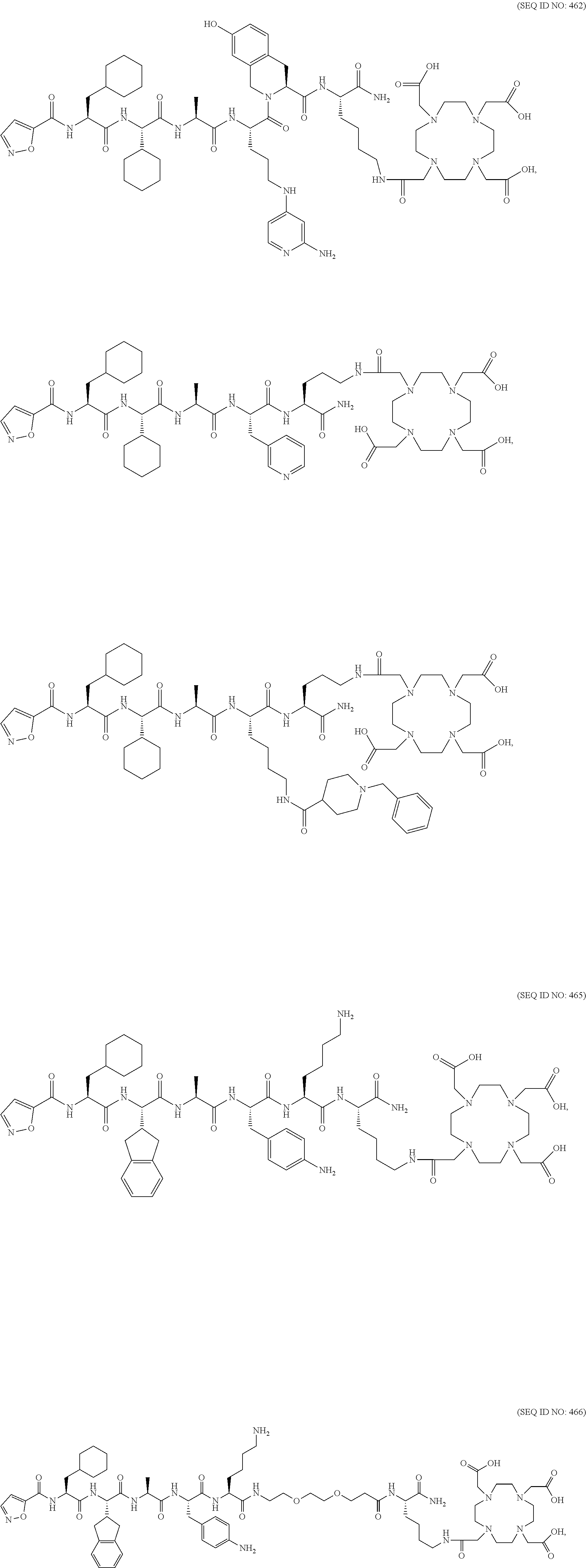

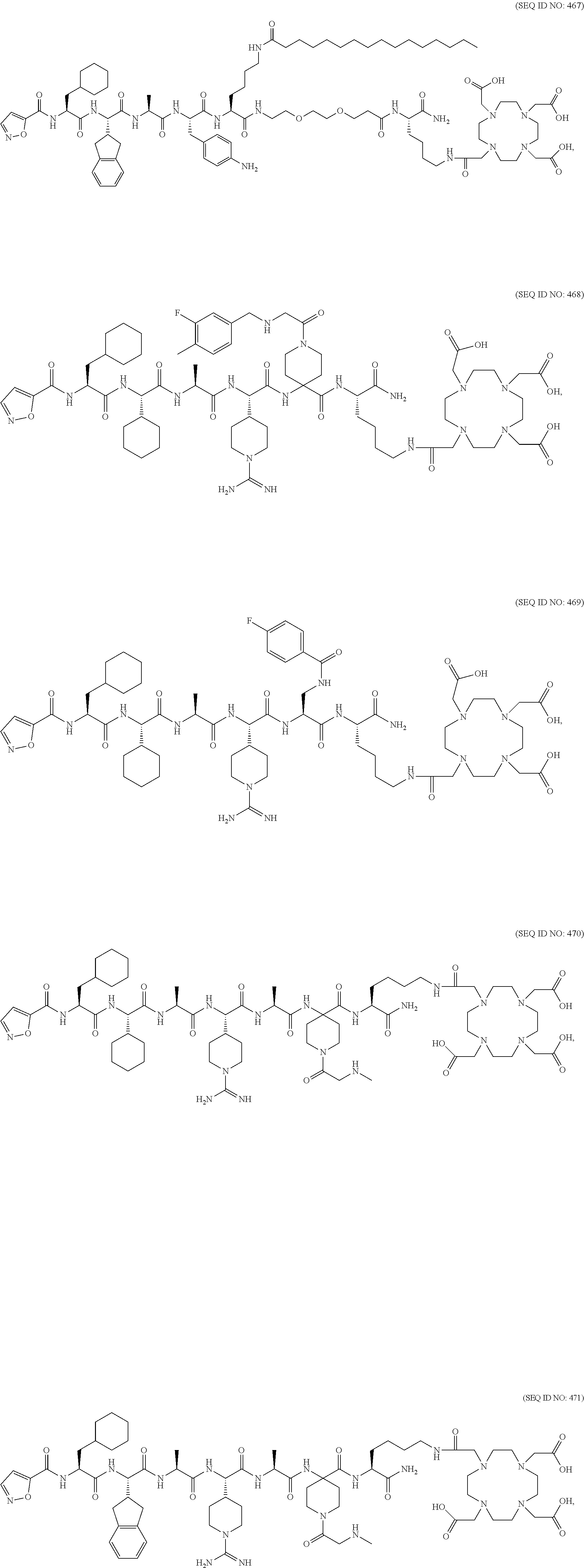

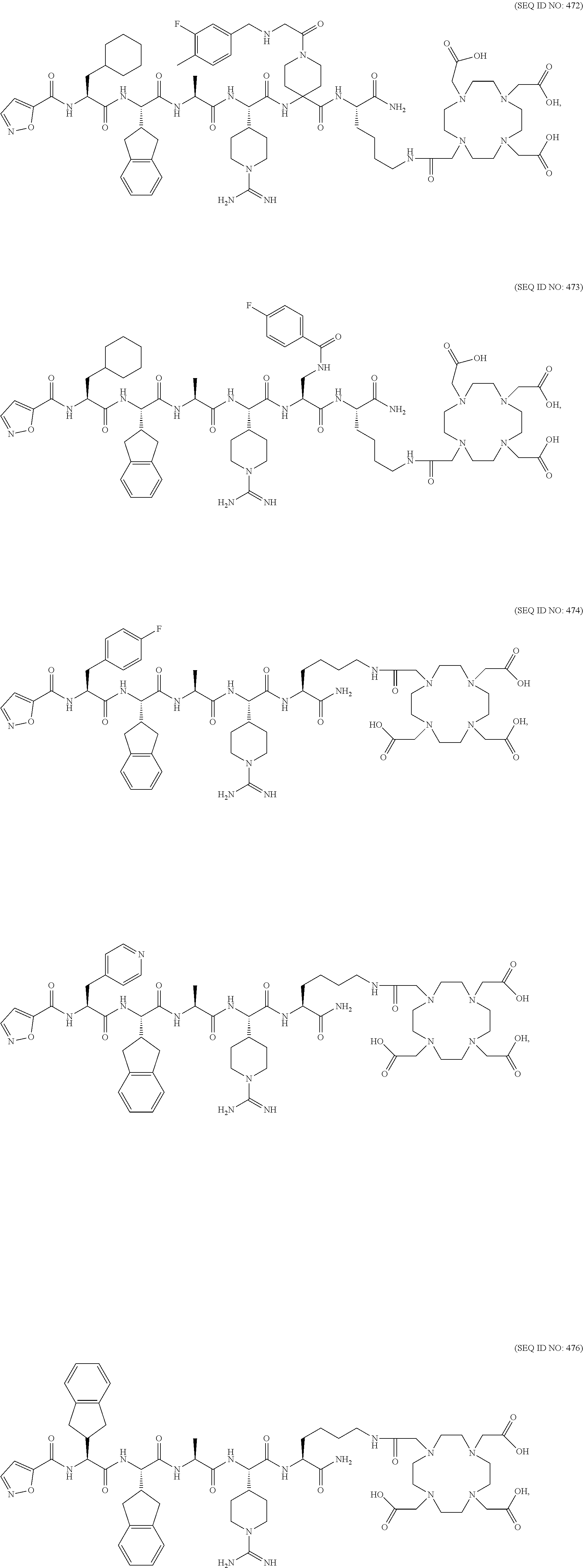

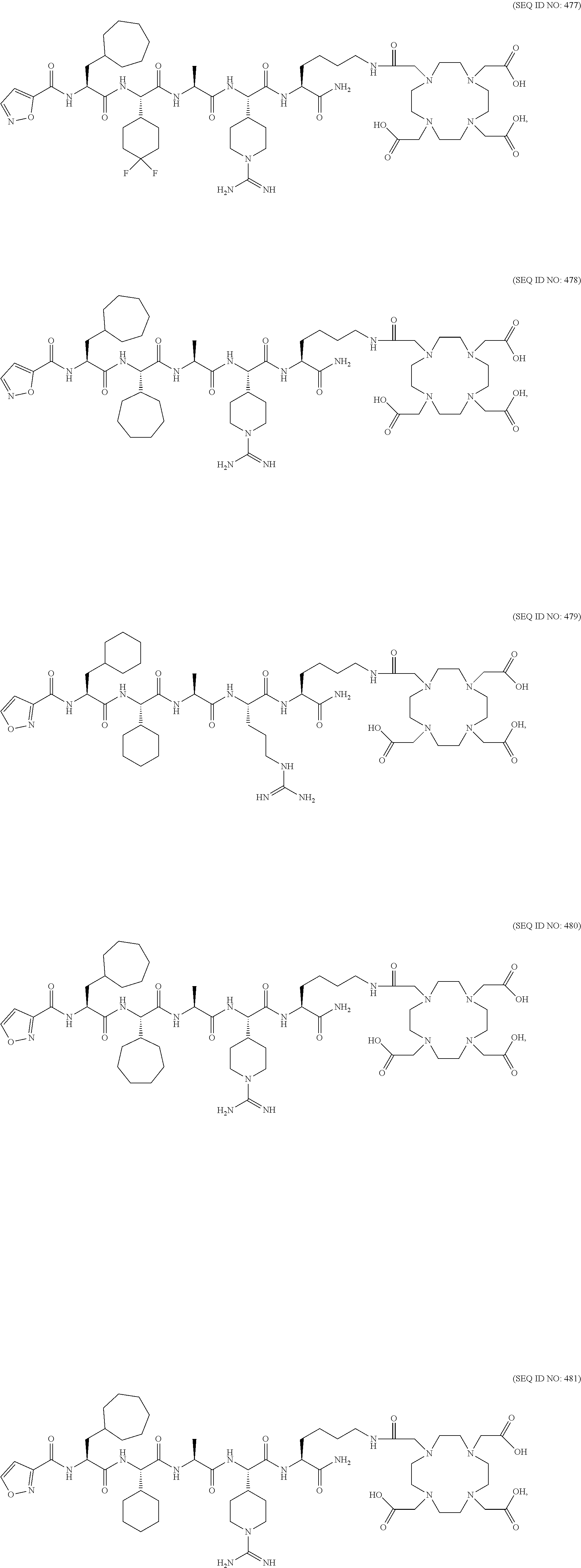

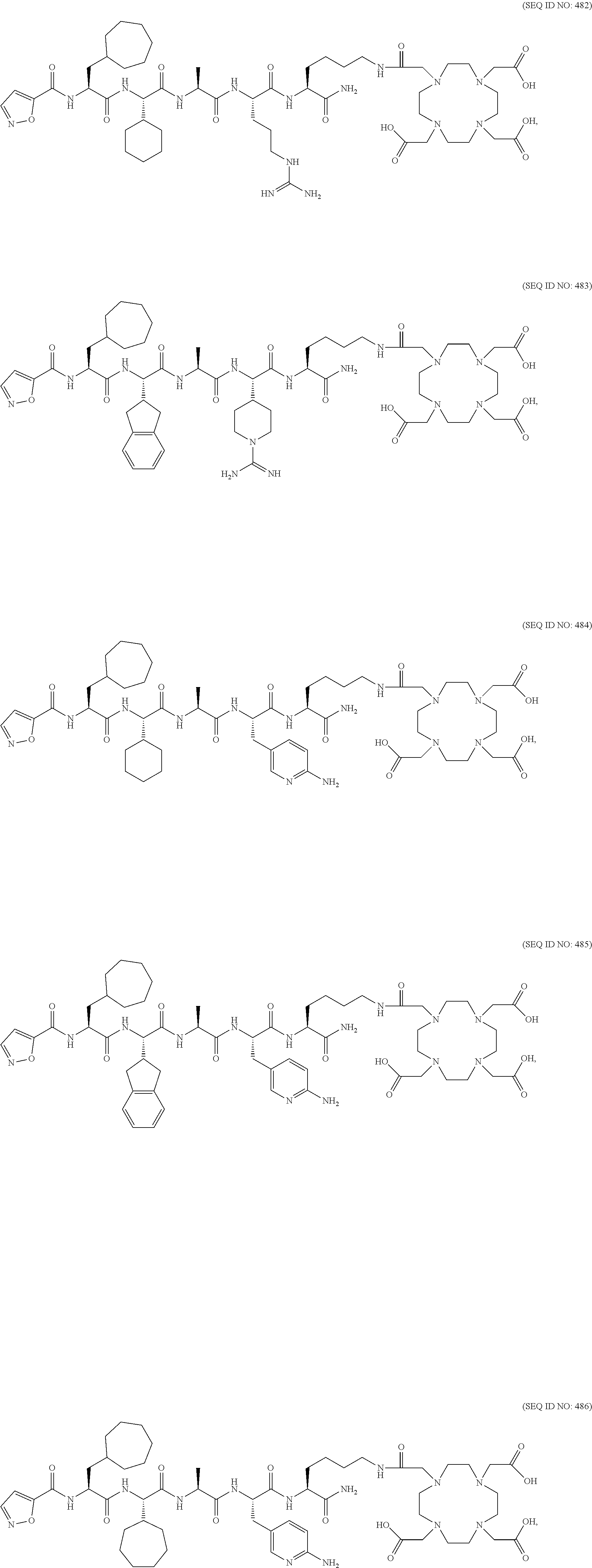

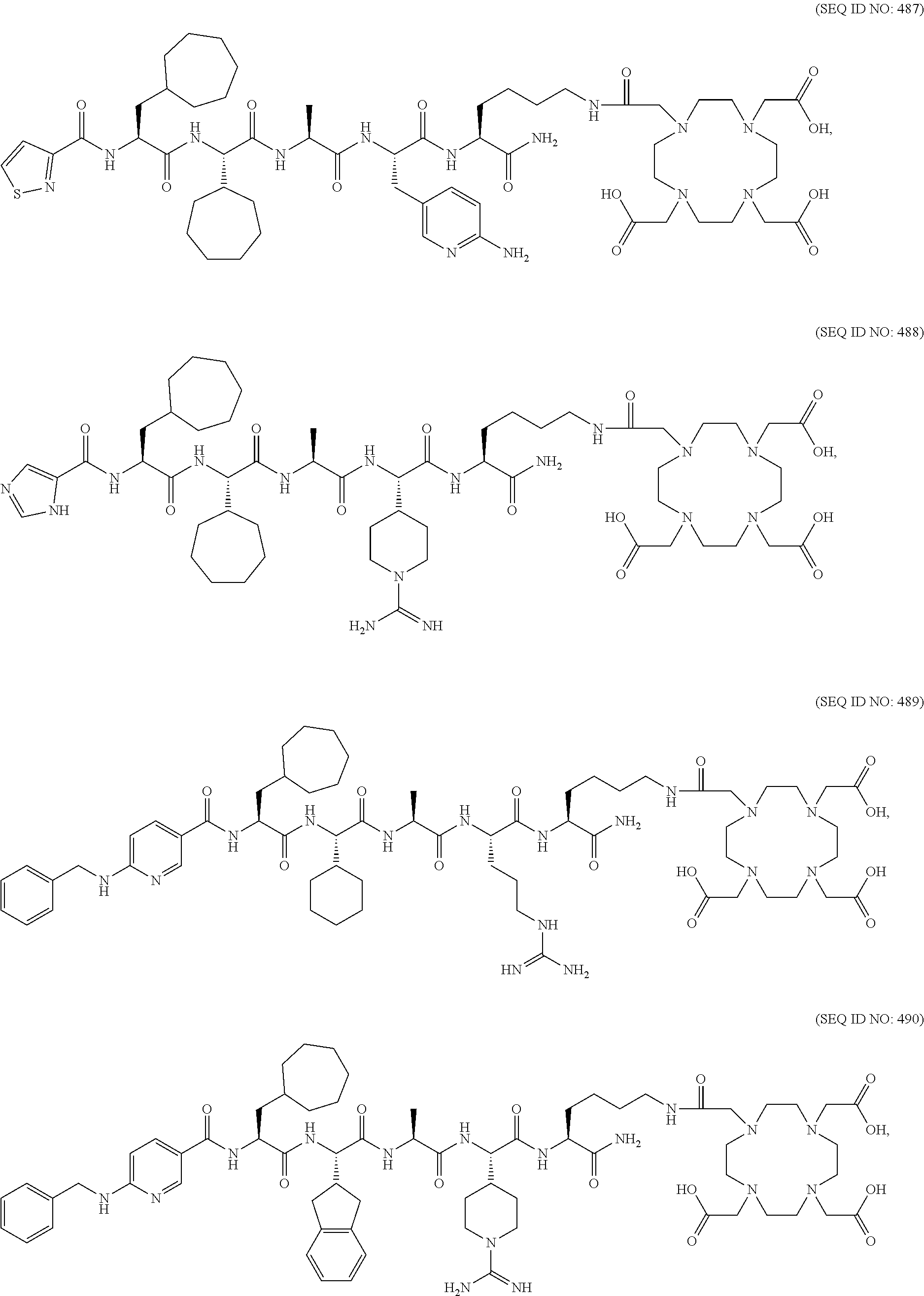

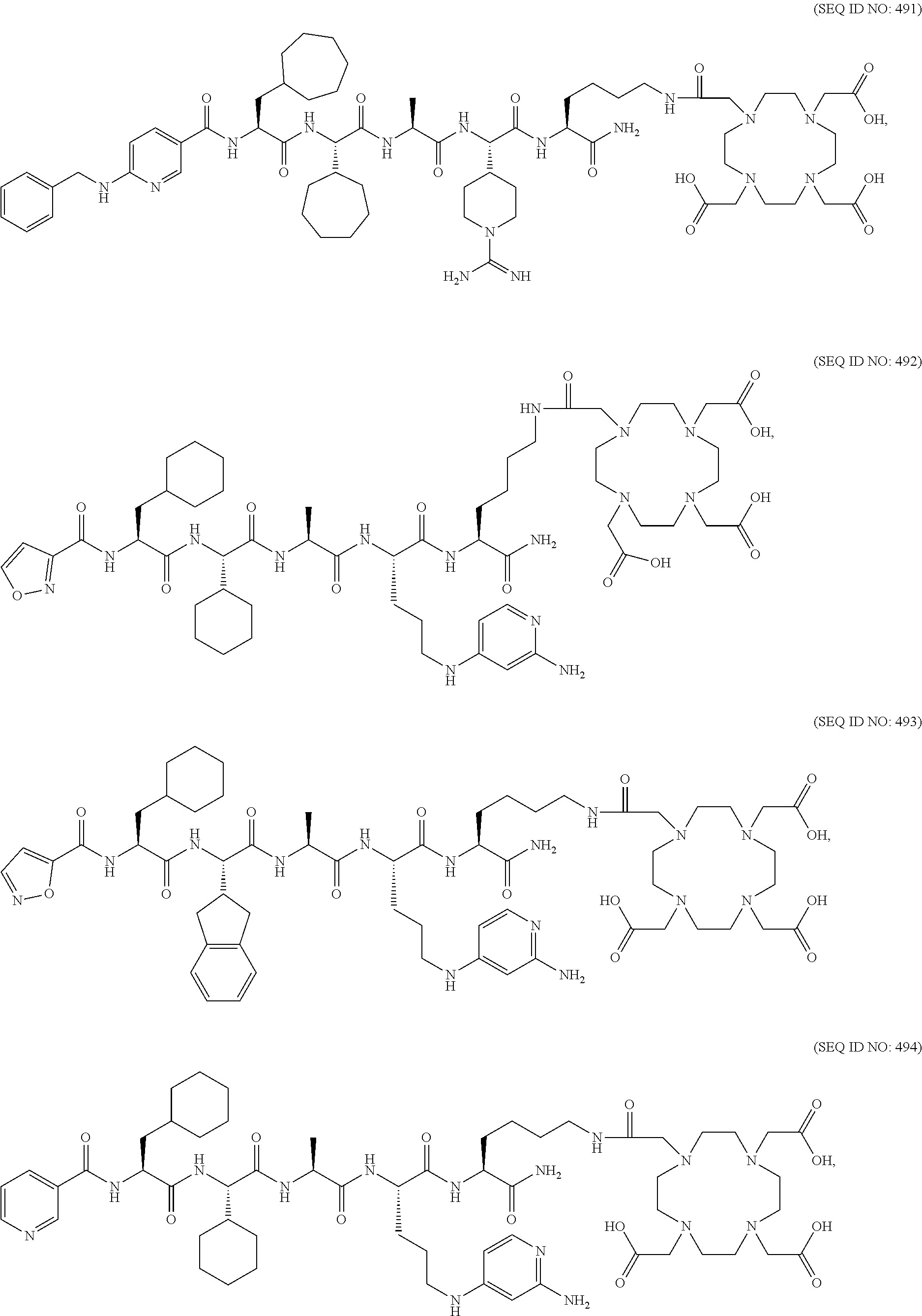

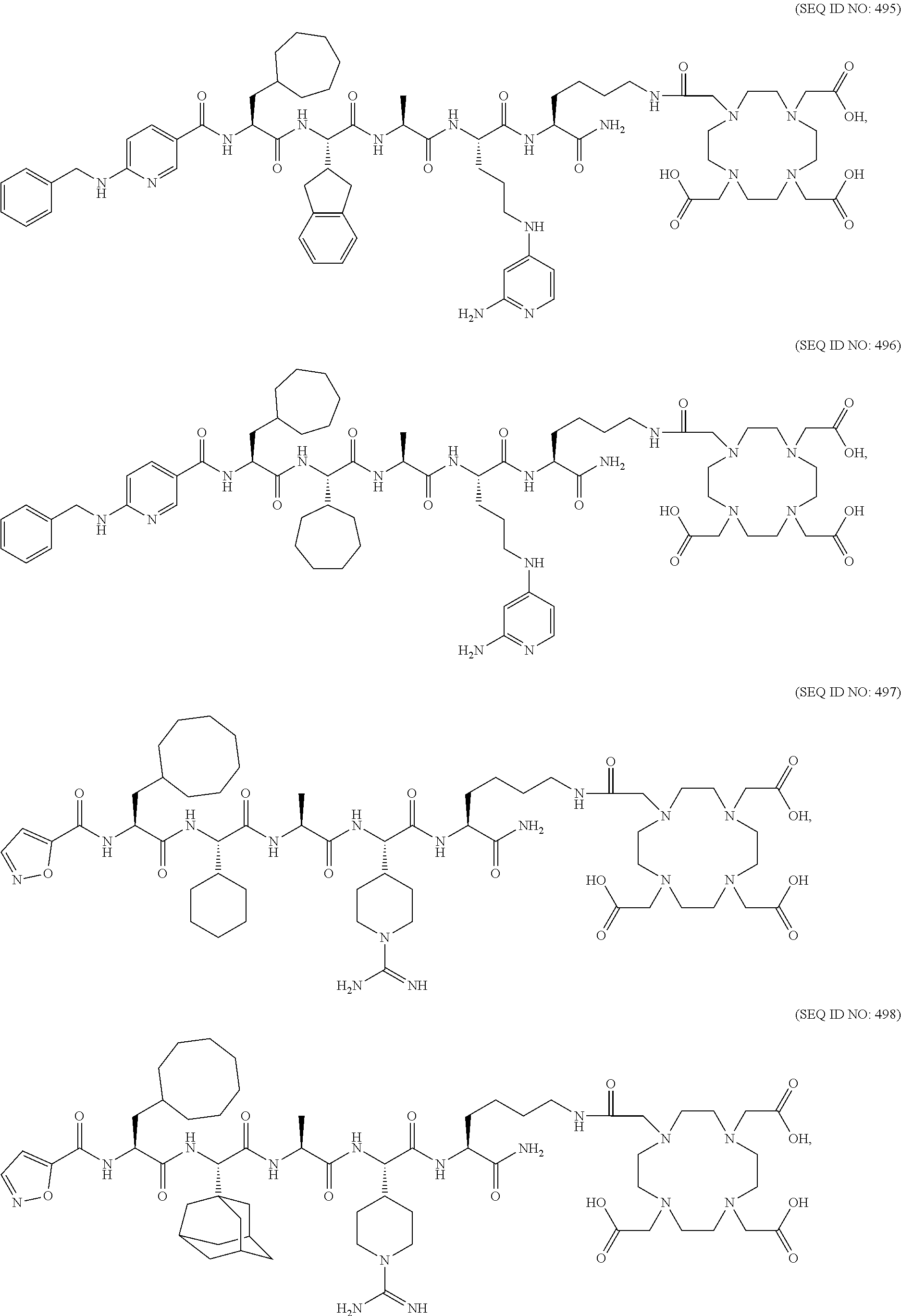

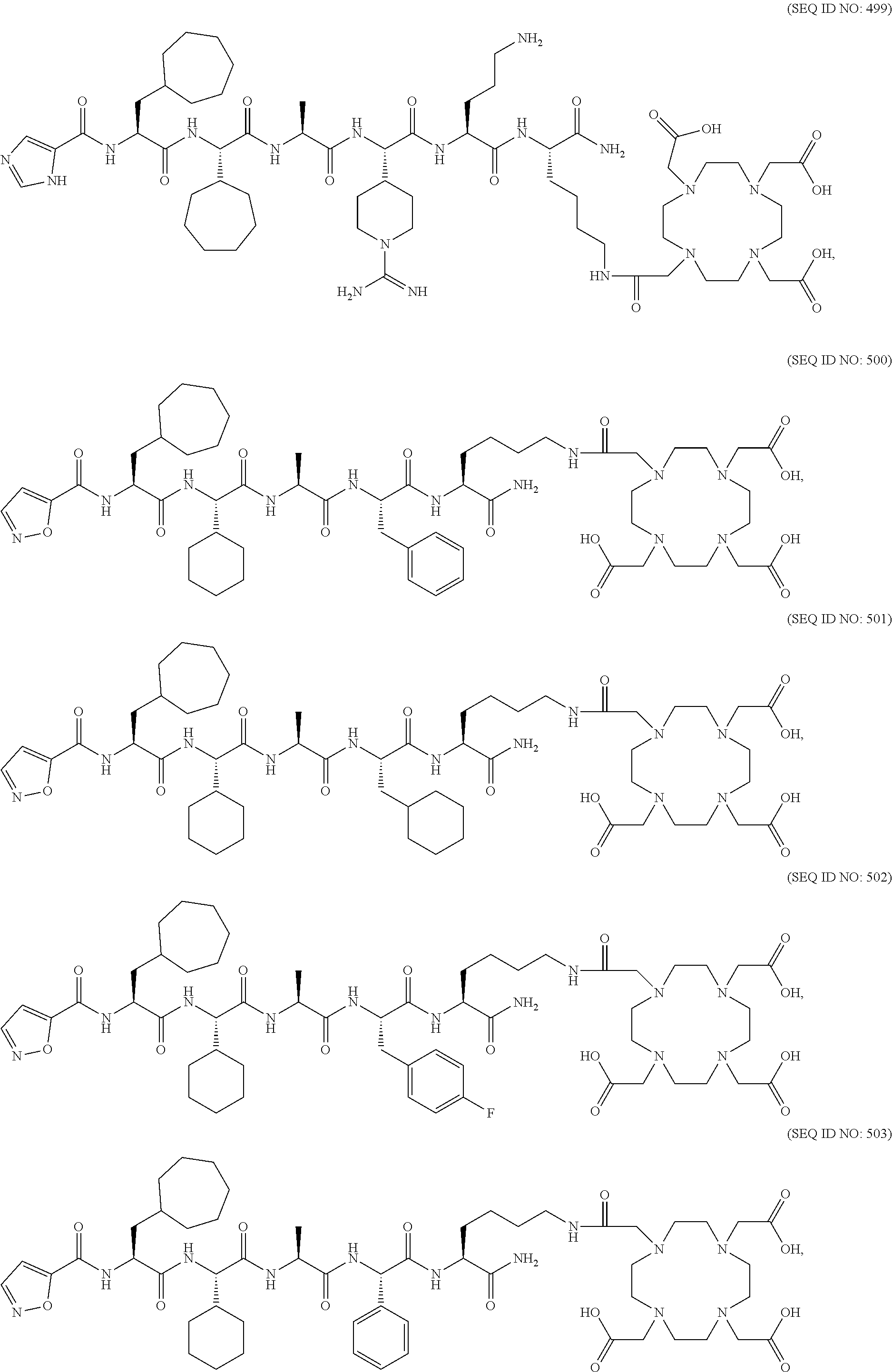

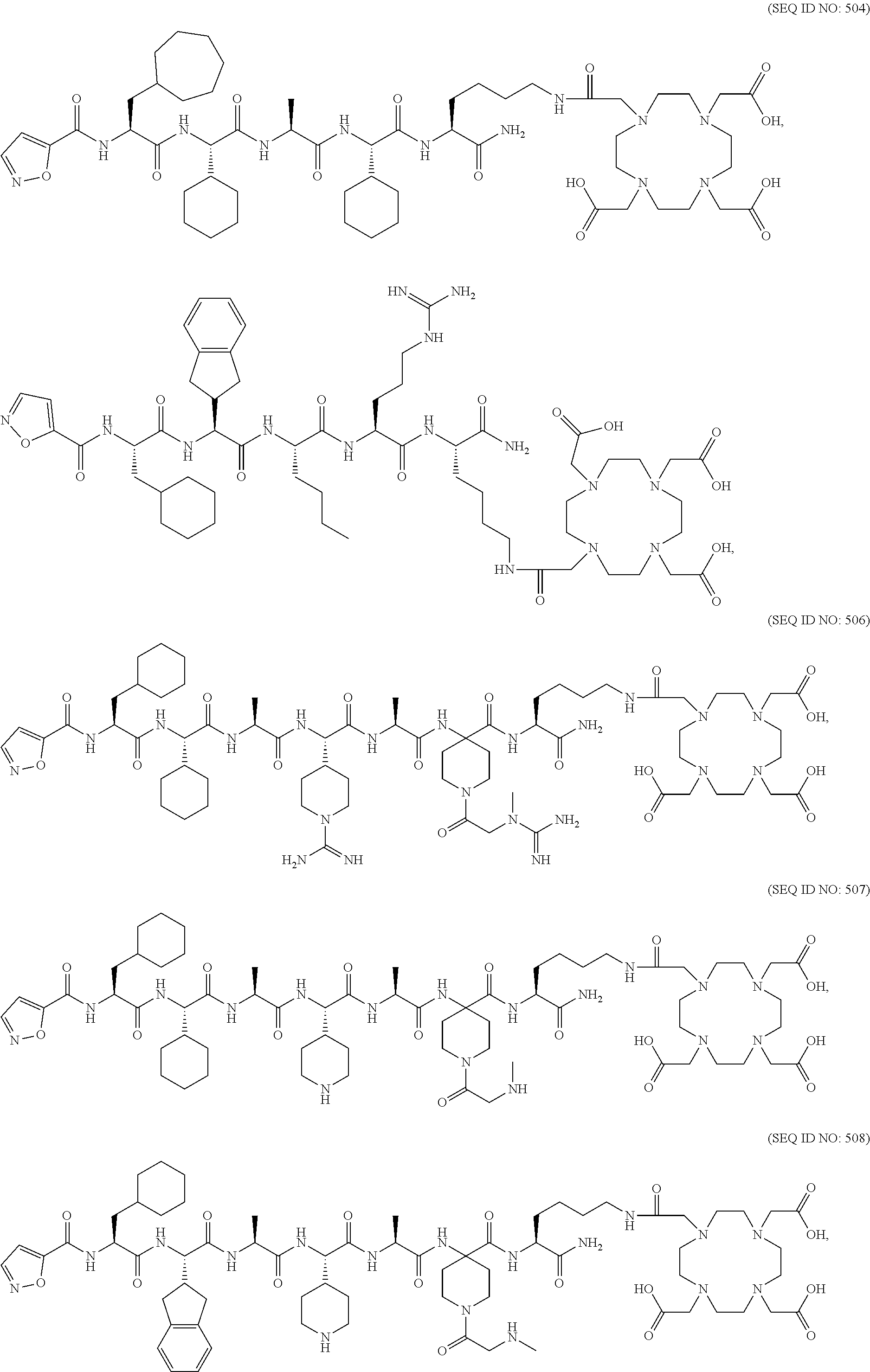

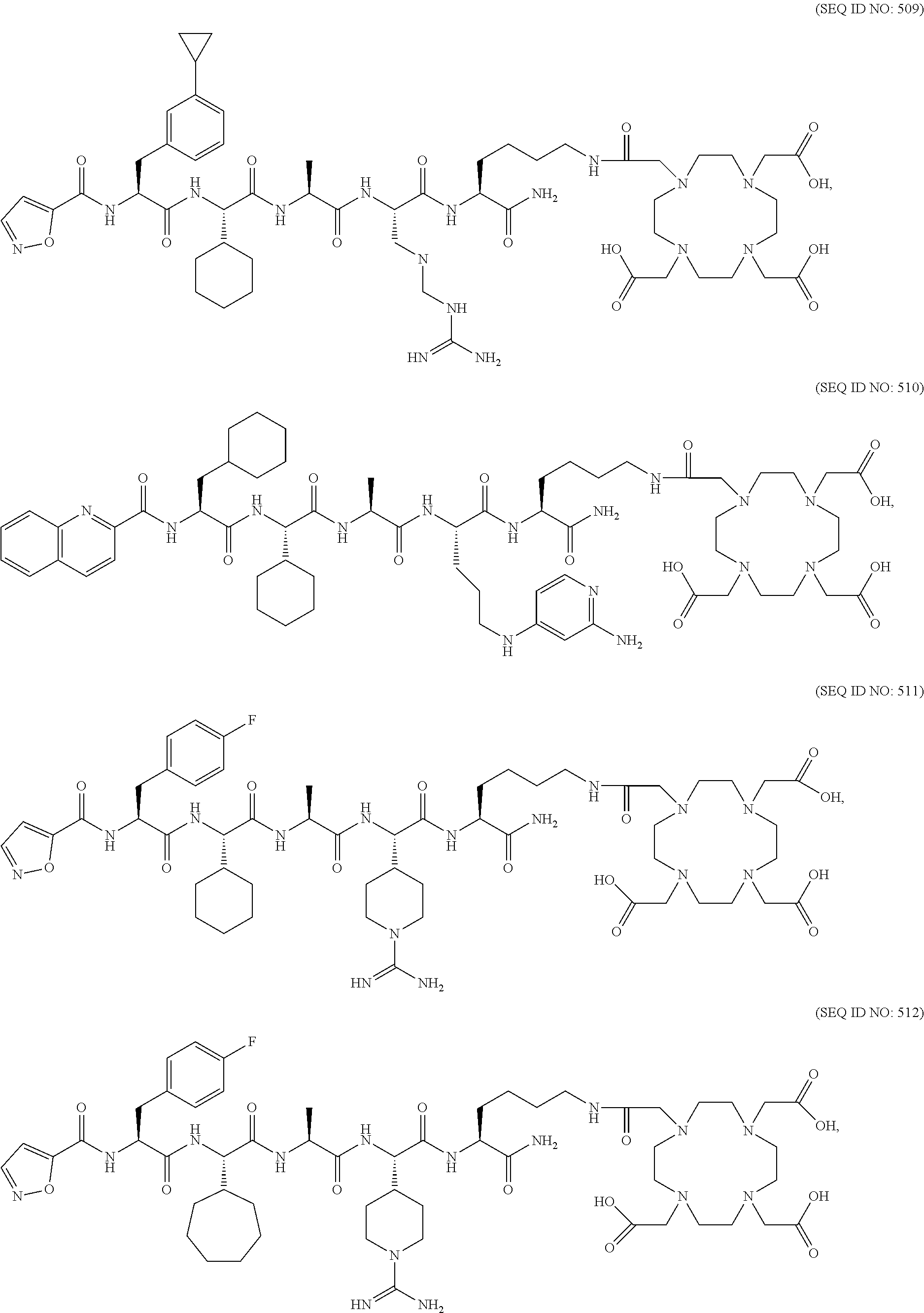

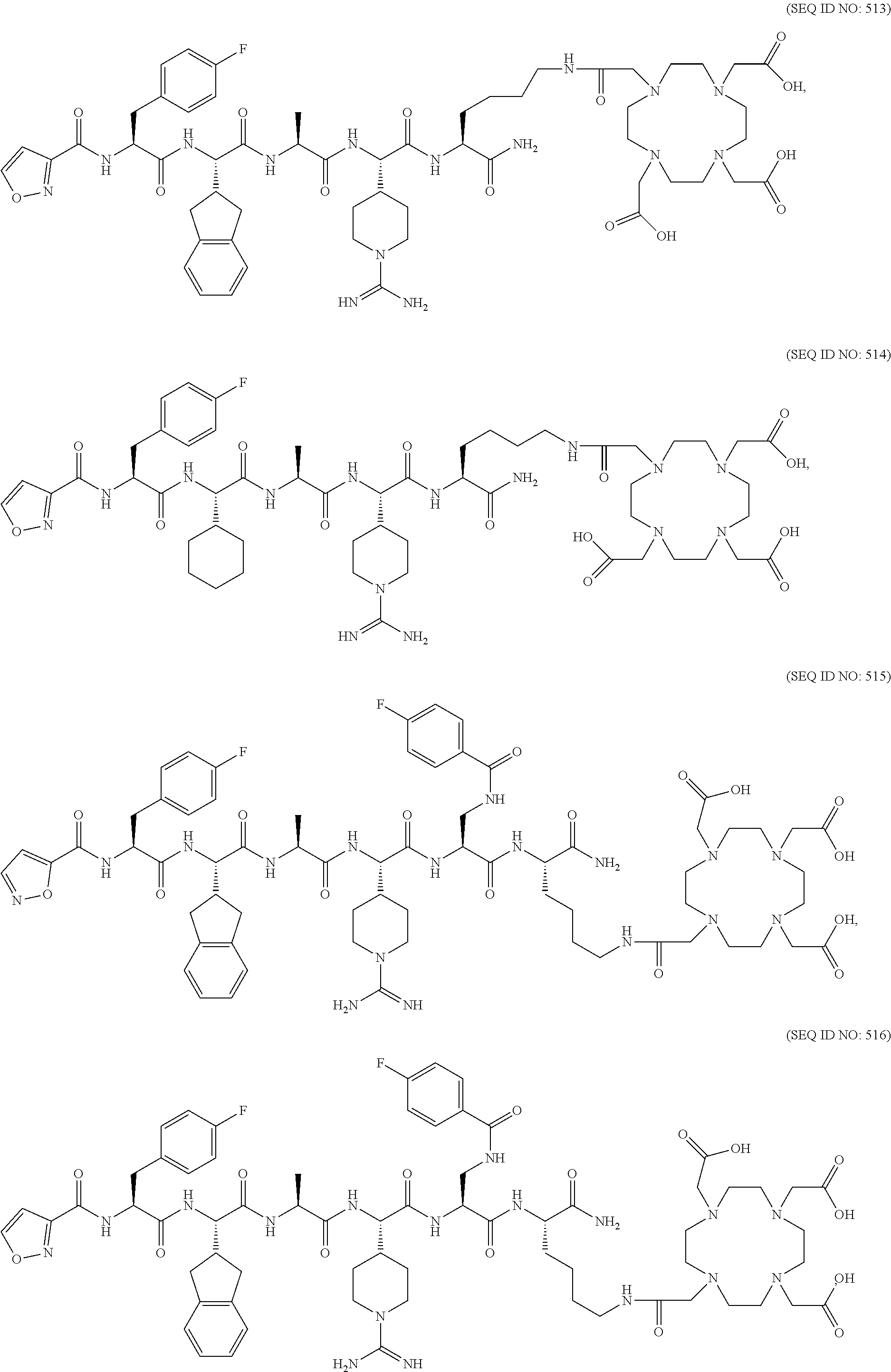

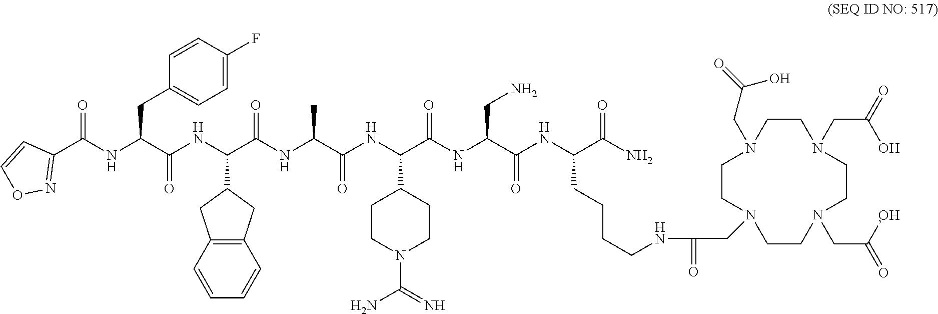 [SMILES: C[C@H](NC(=O)[C@@H](NC(=O)[C@H](Cc1ccc(F)cc1)NC(=O)c1ccon1)C1Cc2ccccc2C1)C(=O)N[C@H](C(=O)N[C@@H](CN)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

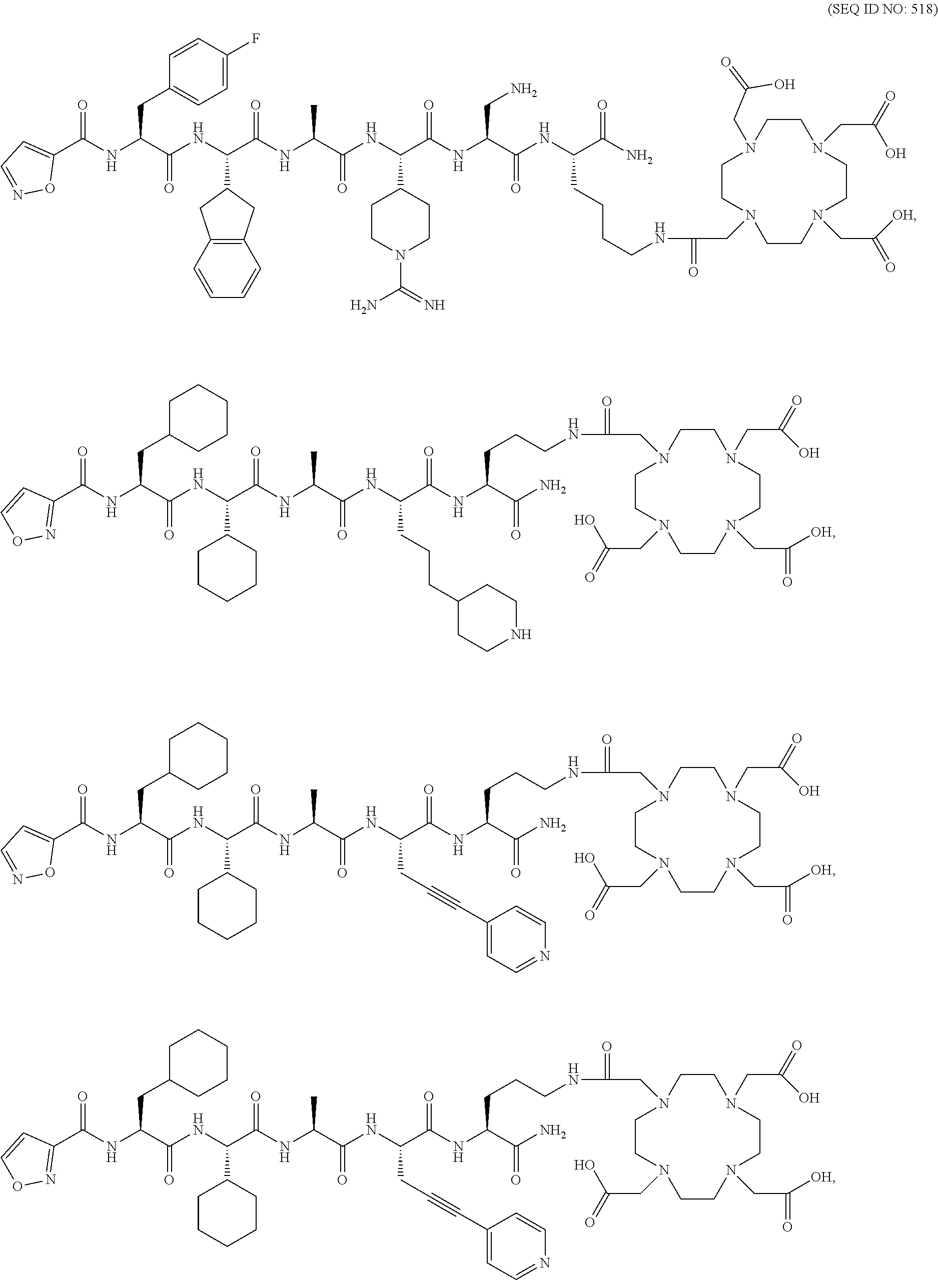

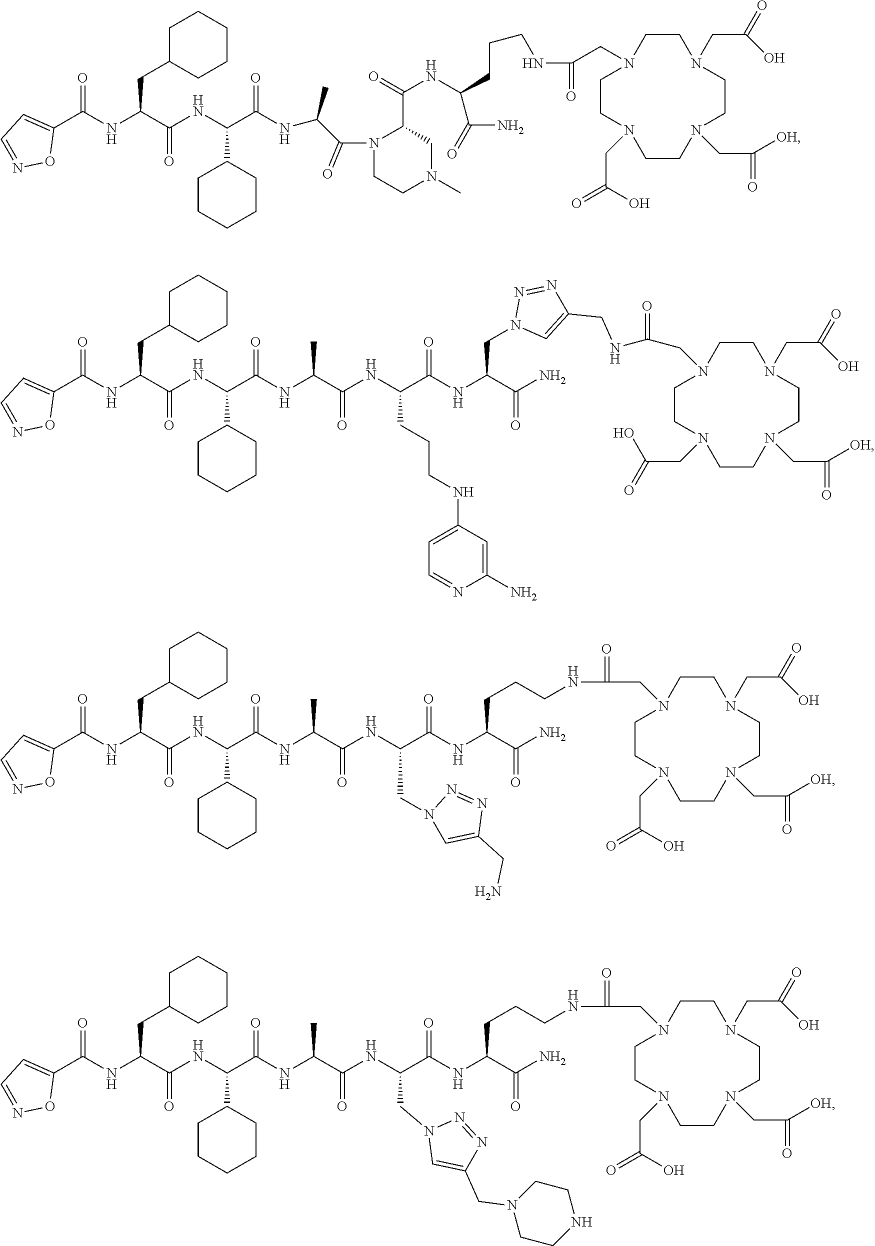

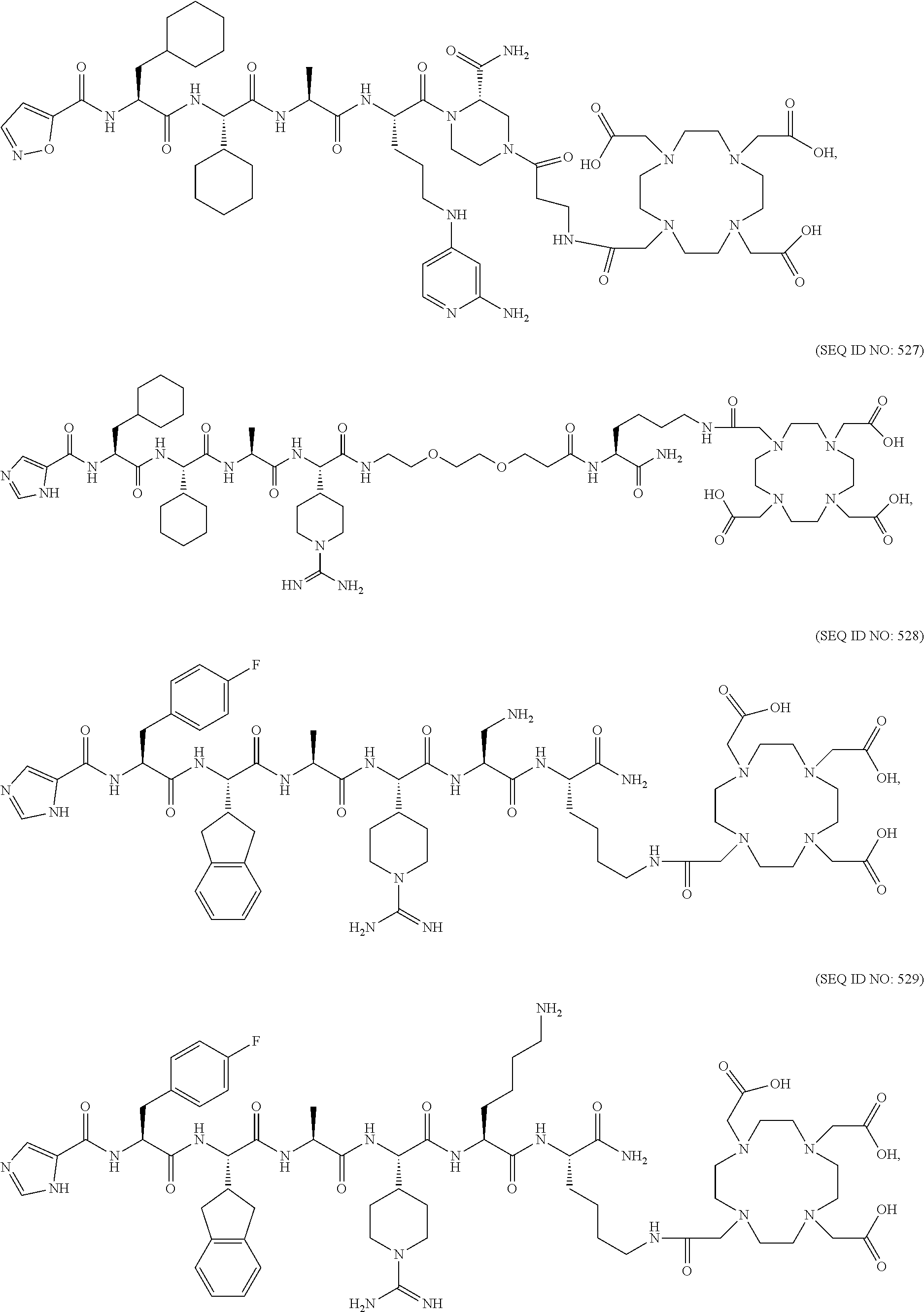

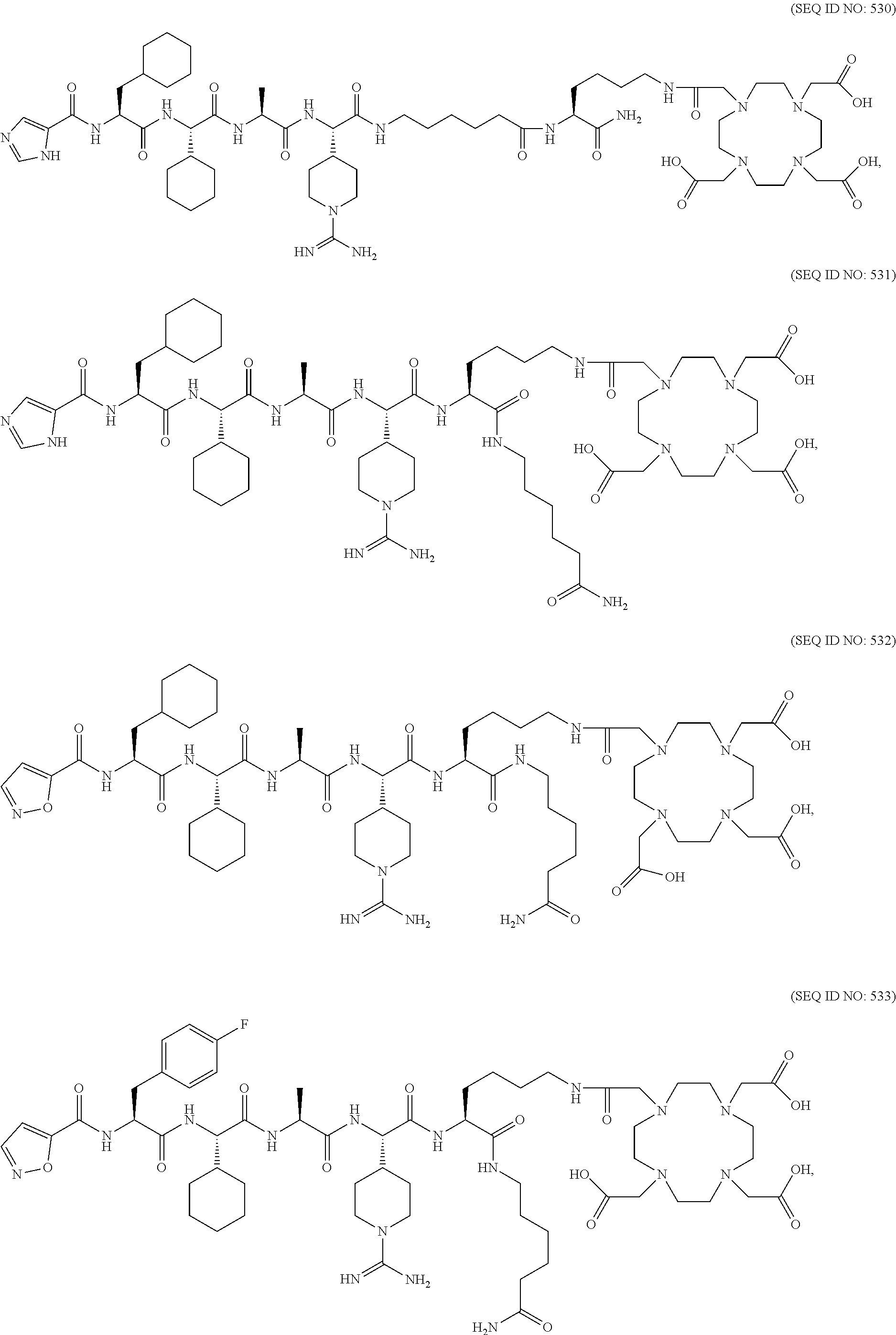

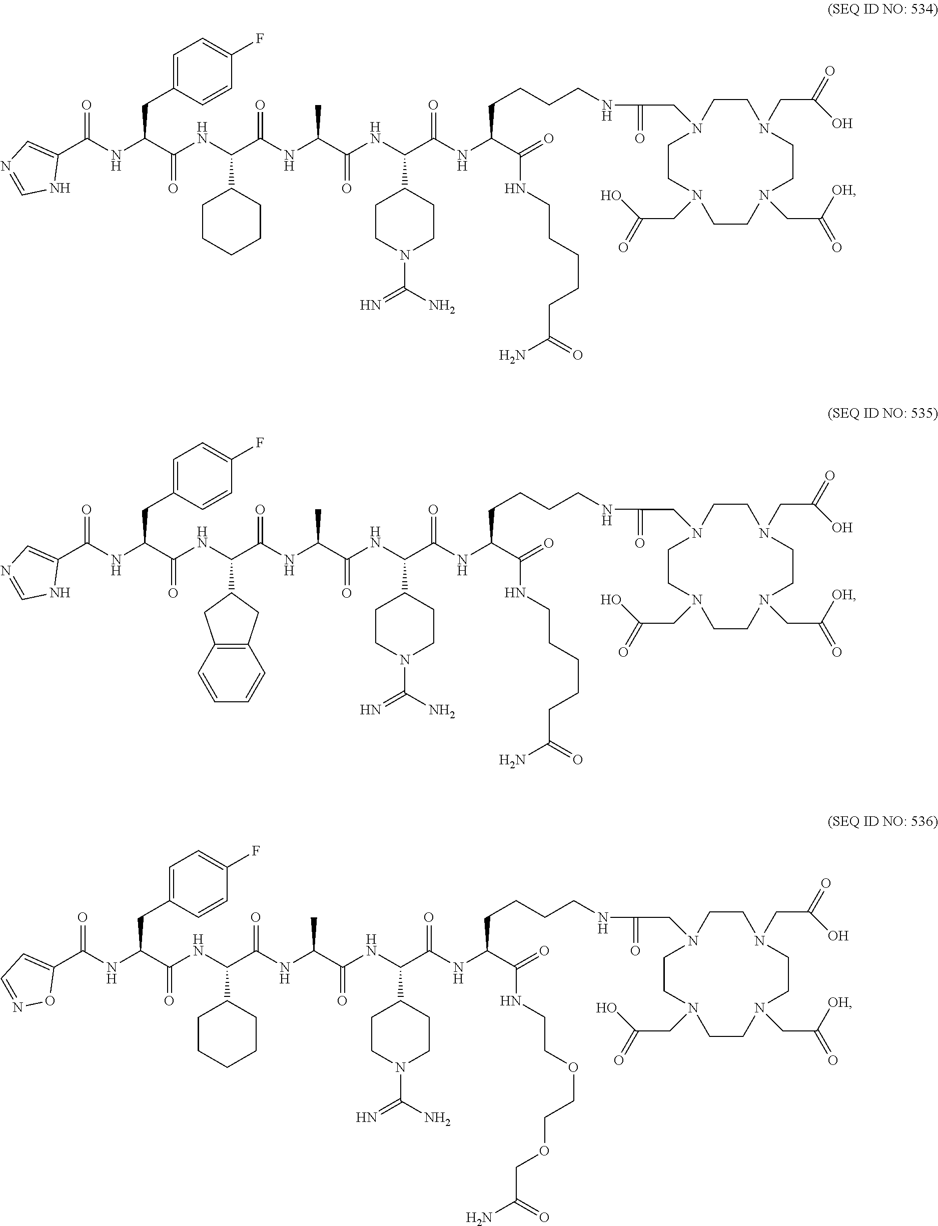

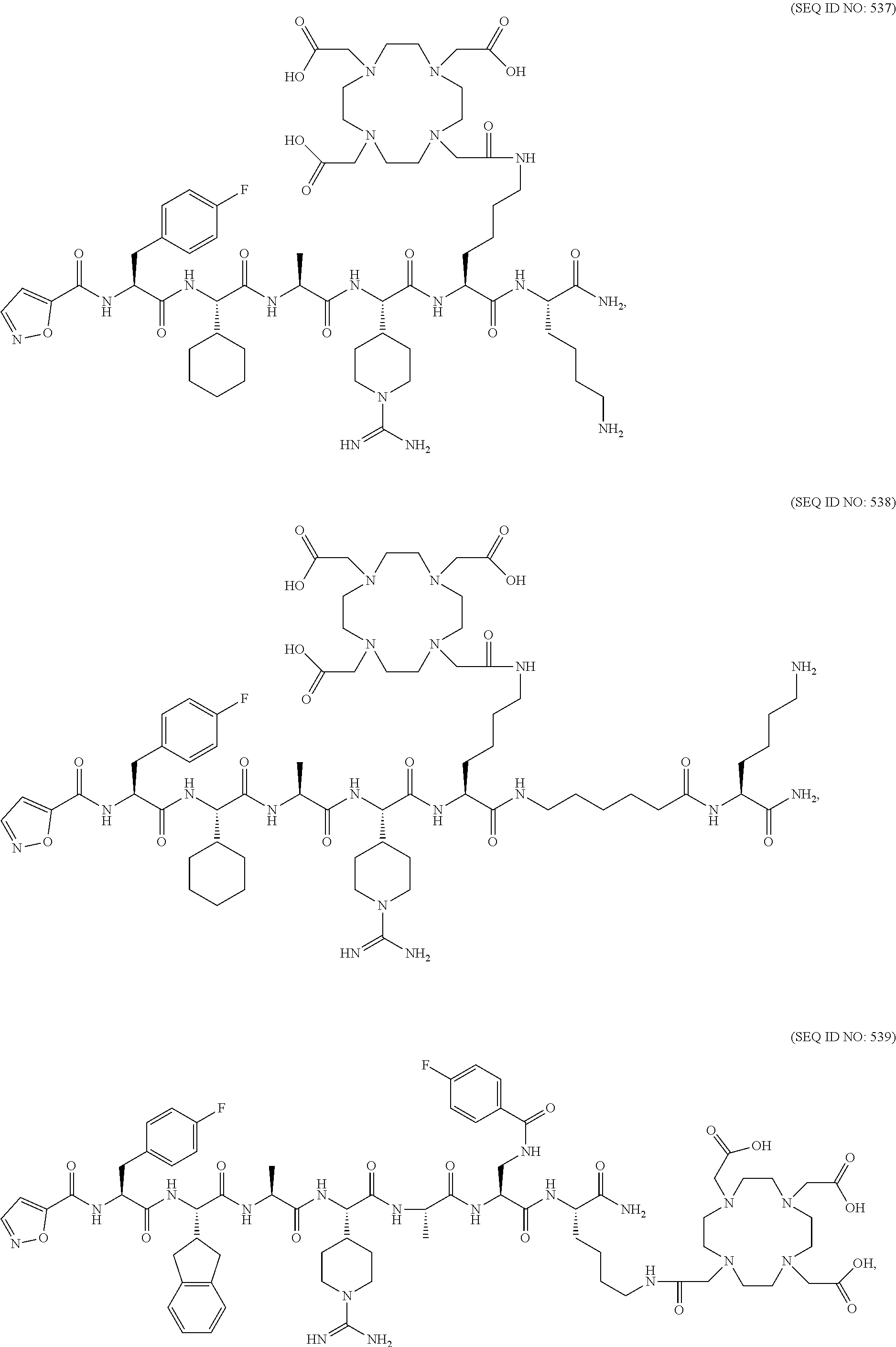

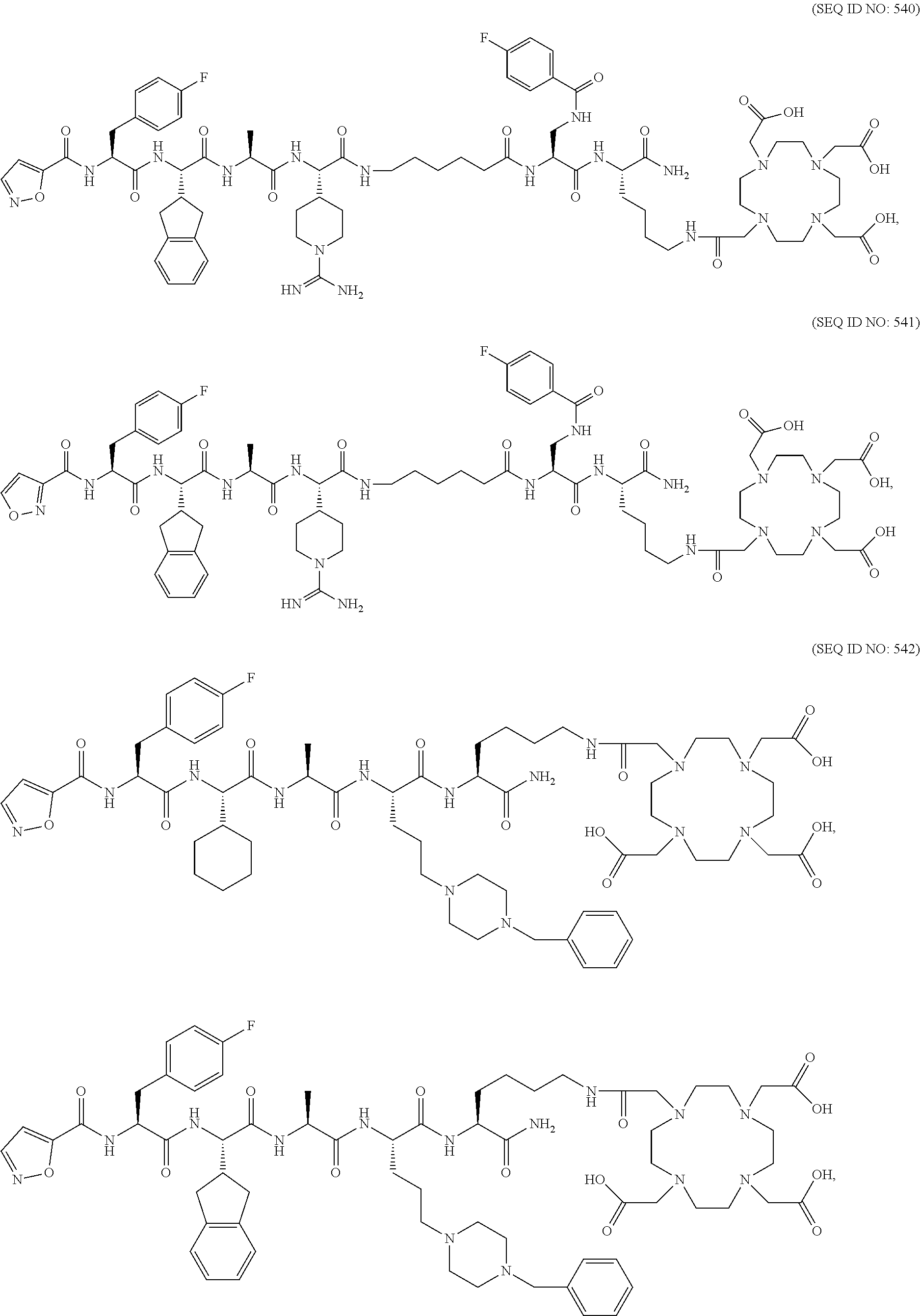

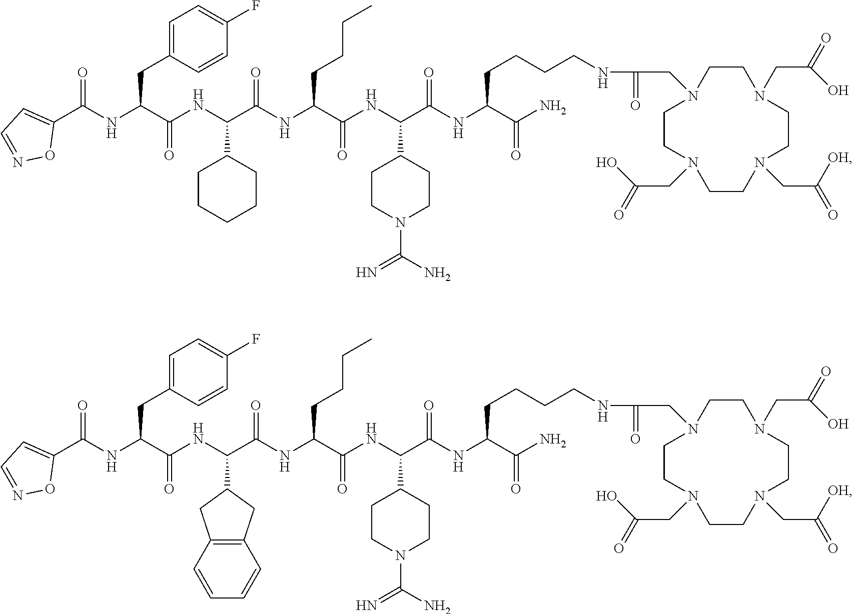 [SMILES: CCCC[C@H](NC(=O)[C@@H](NC(=O)[C@H](Cc1ccc(F)cc1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; CCCC[C@H](NC(=O)[C@@H](NC(=O)[C@H](Cc1ccc(F)cc1)NC(=O)c1ccno1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

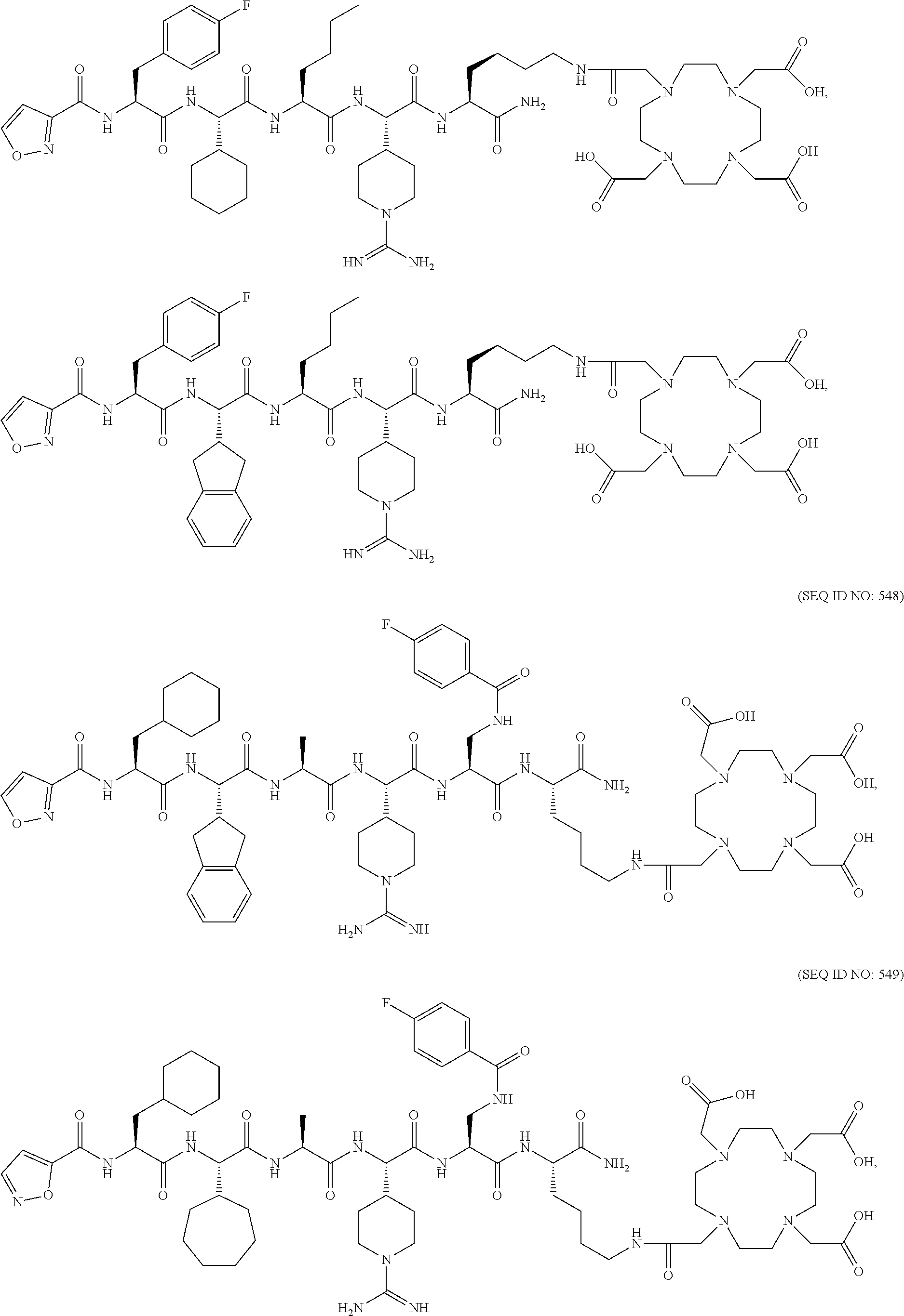

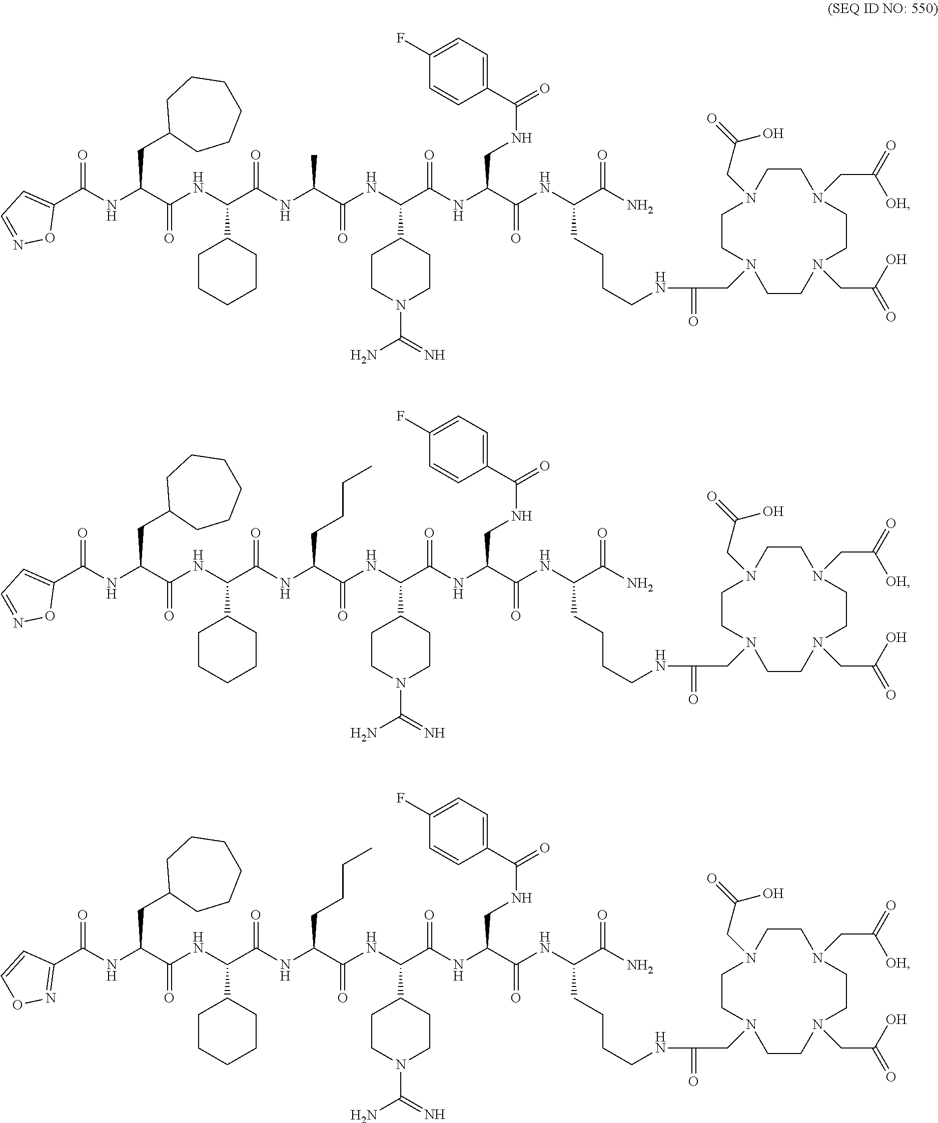

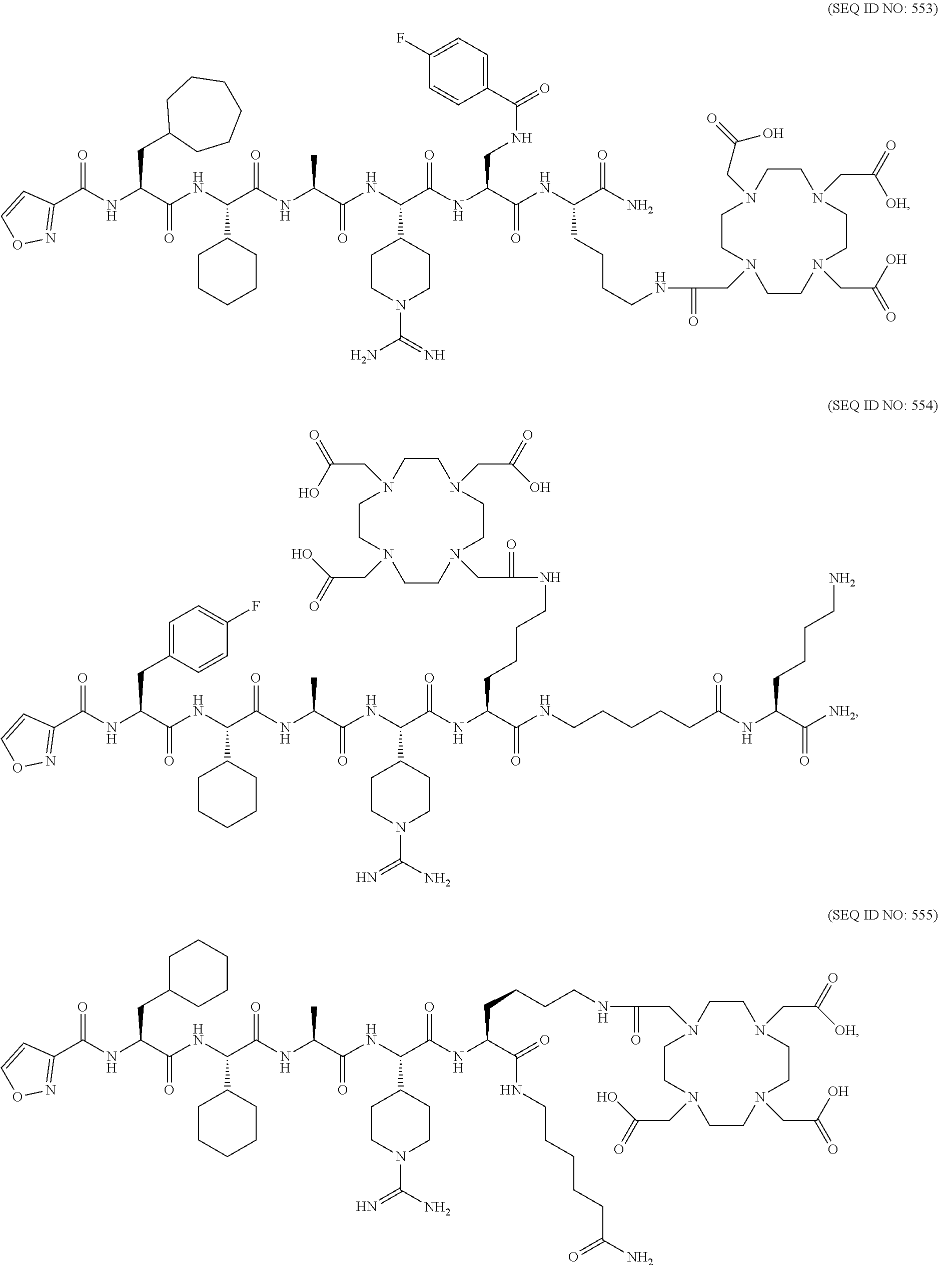

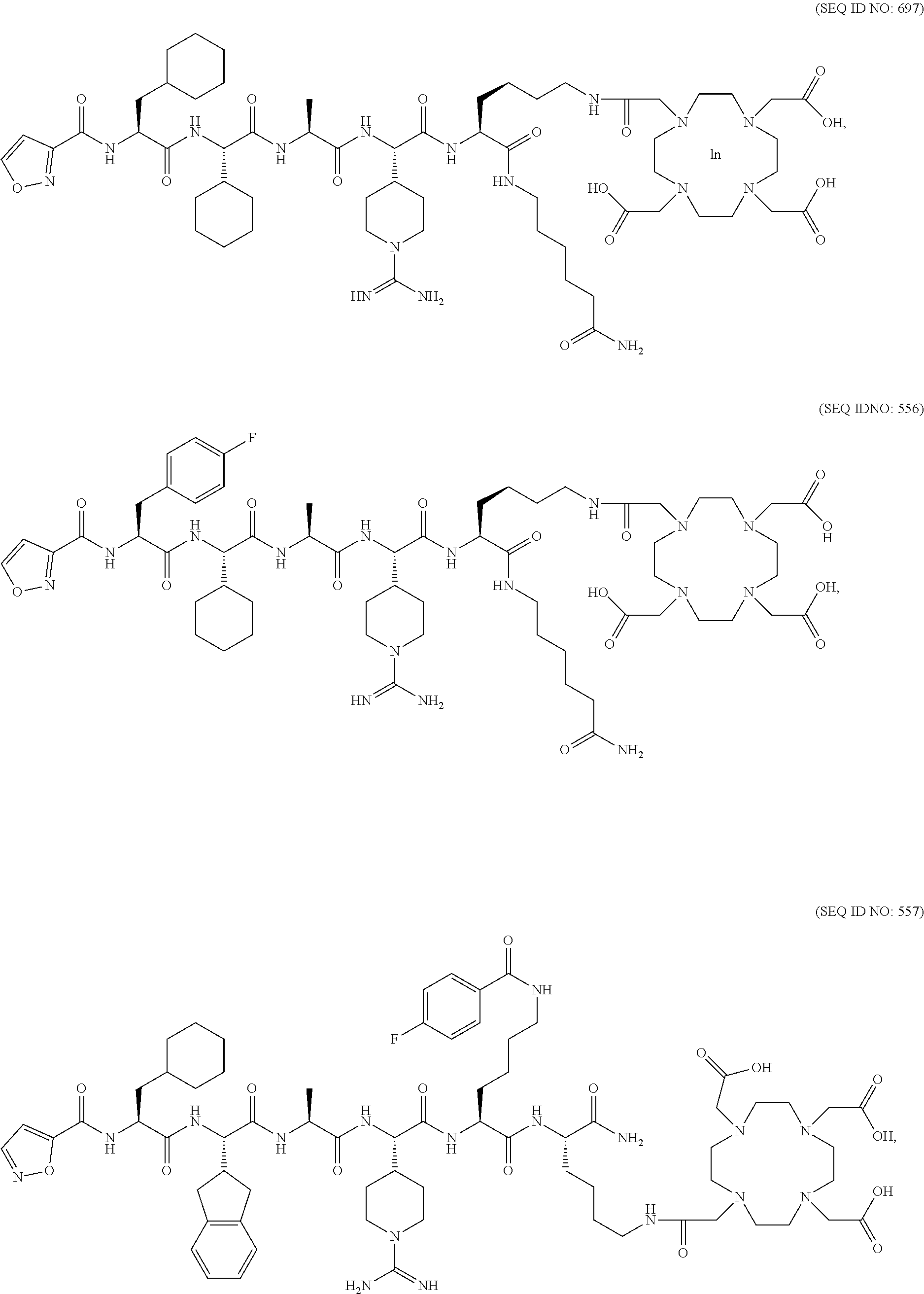

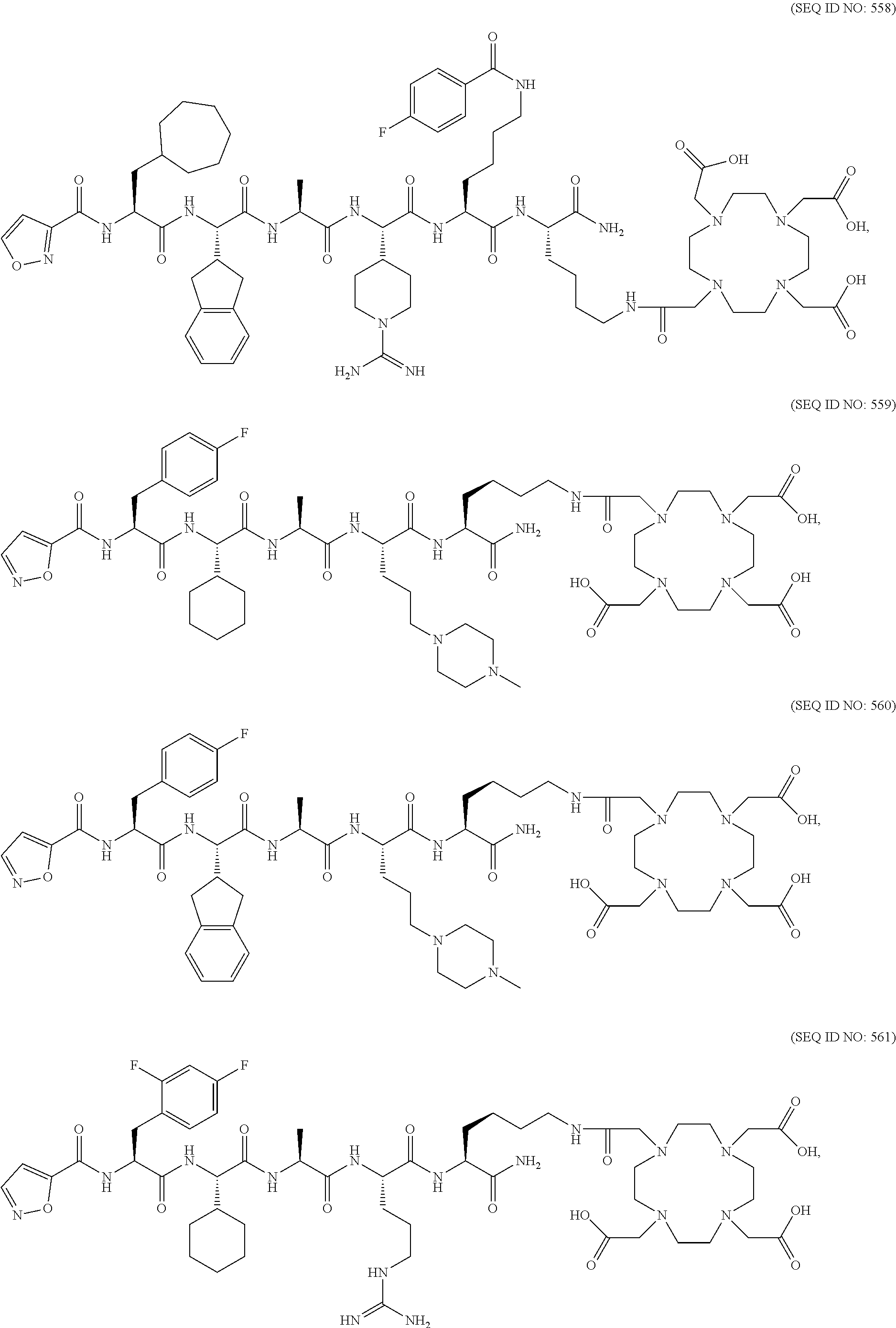

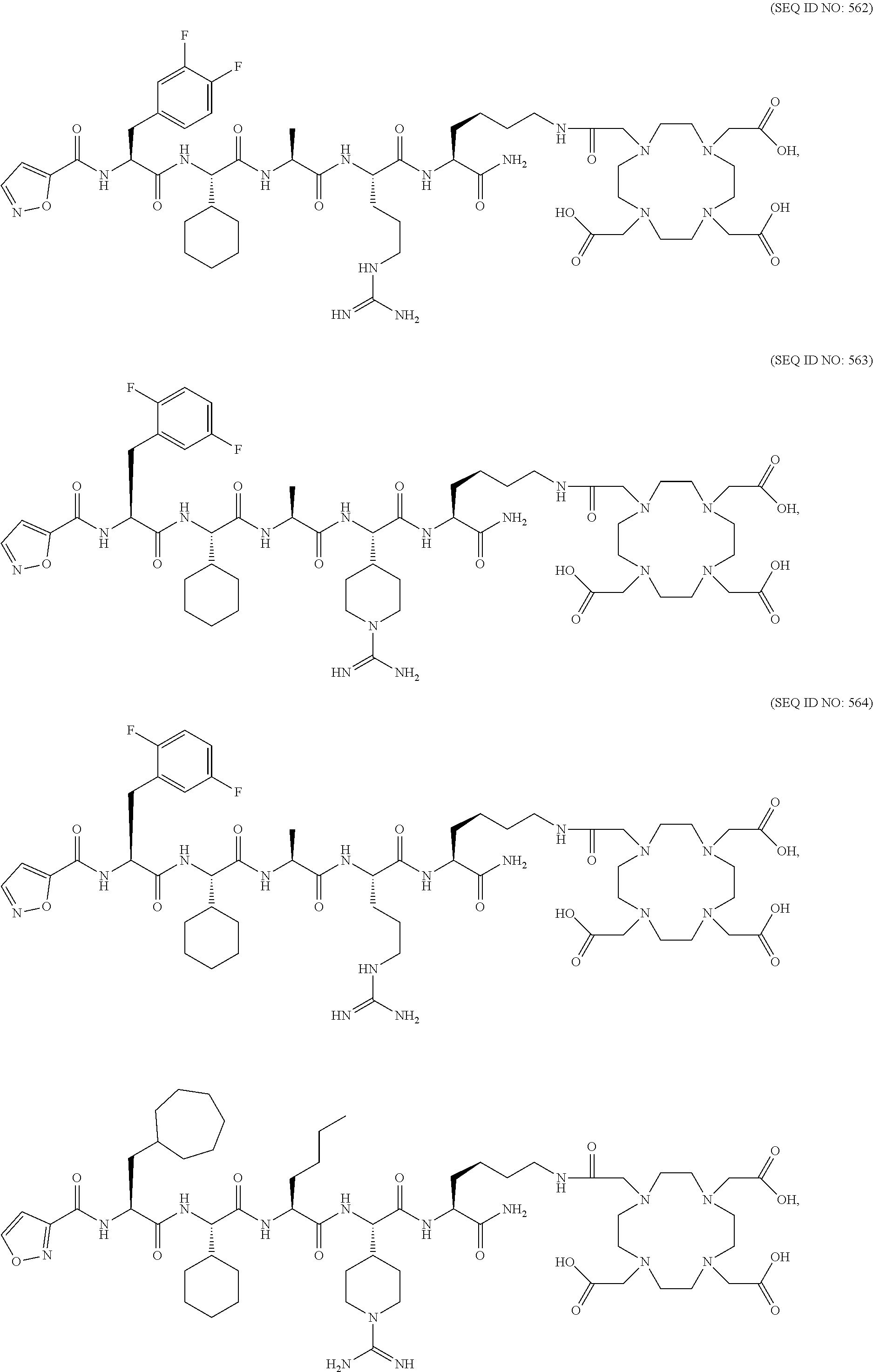

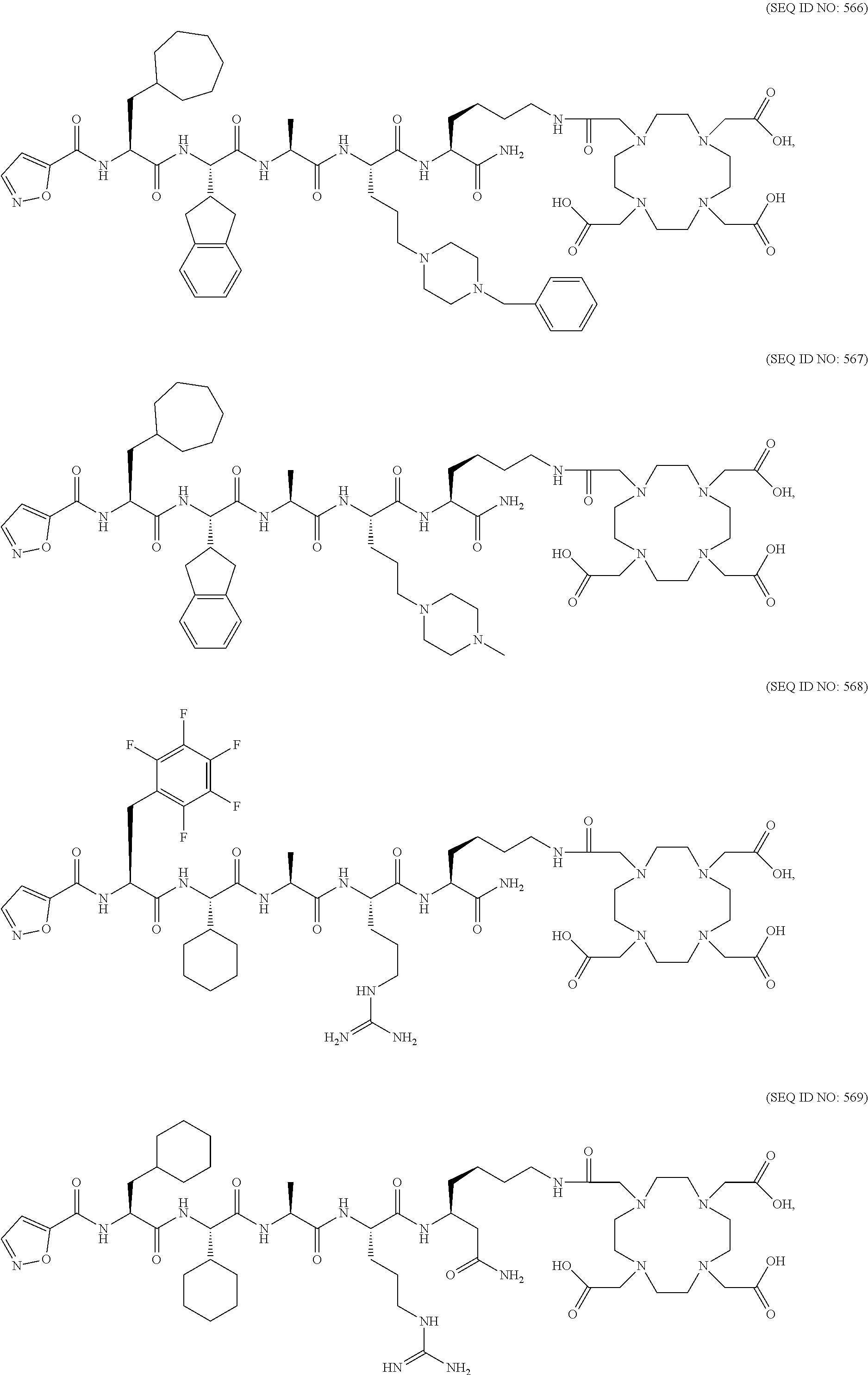

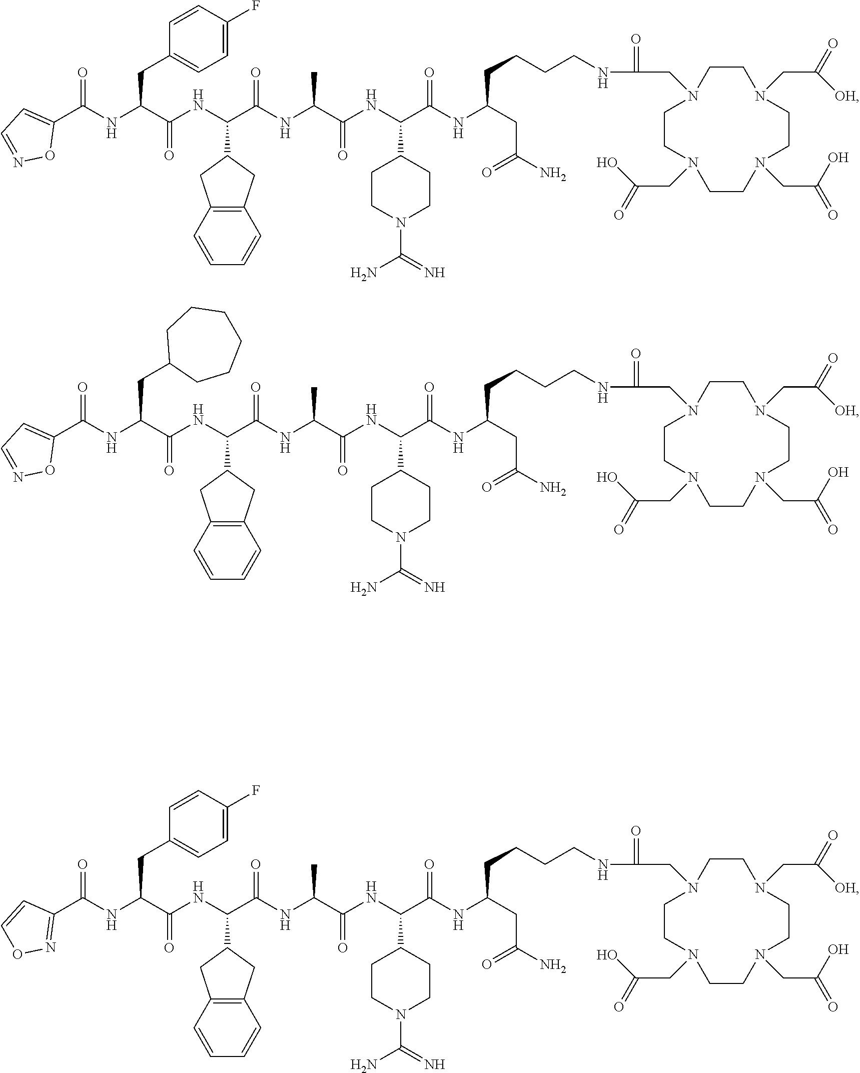

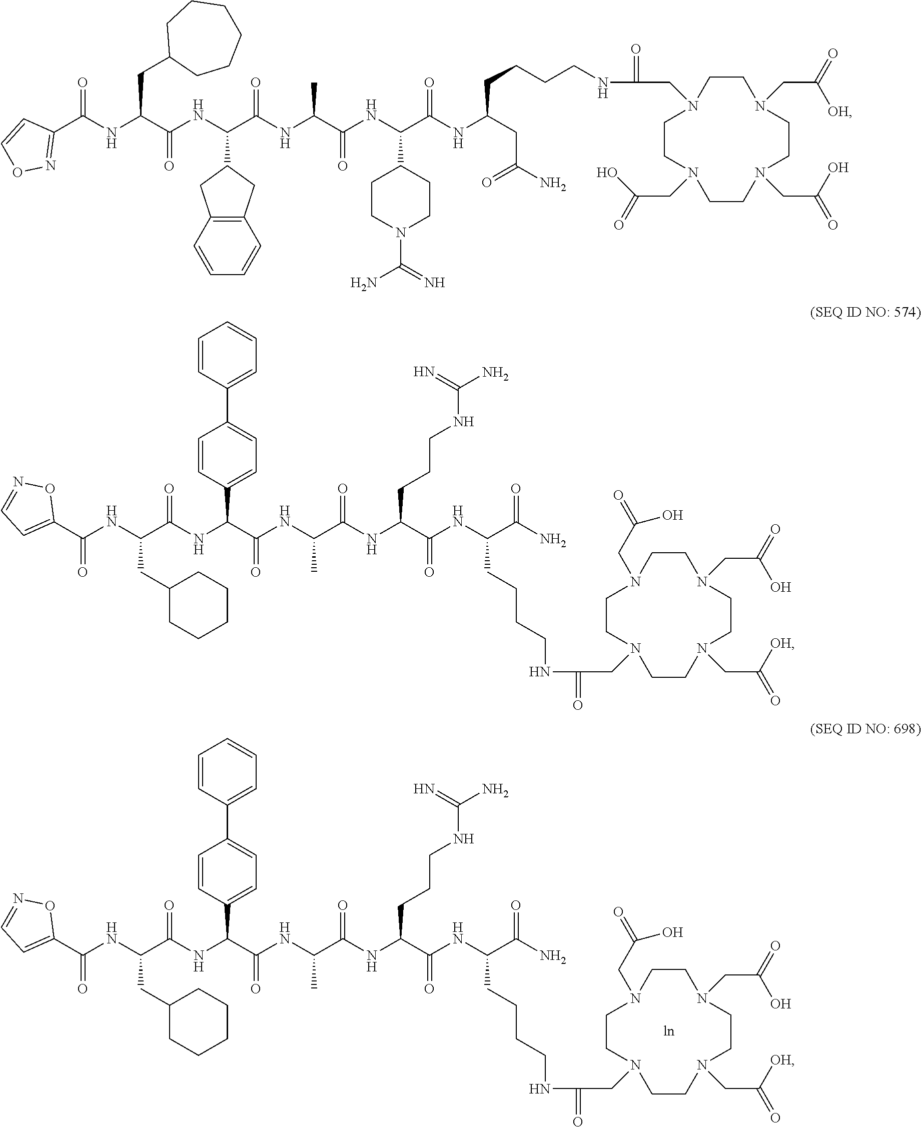

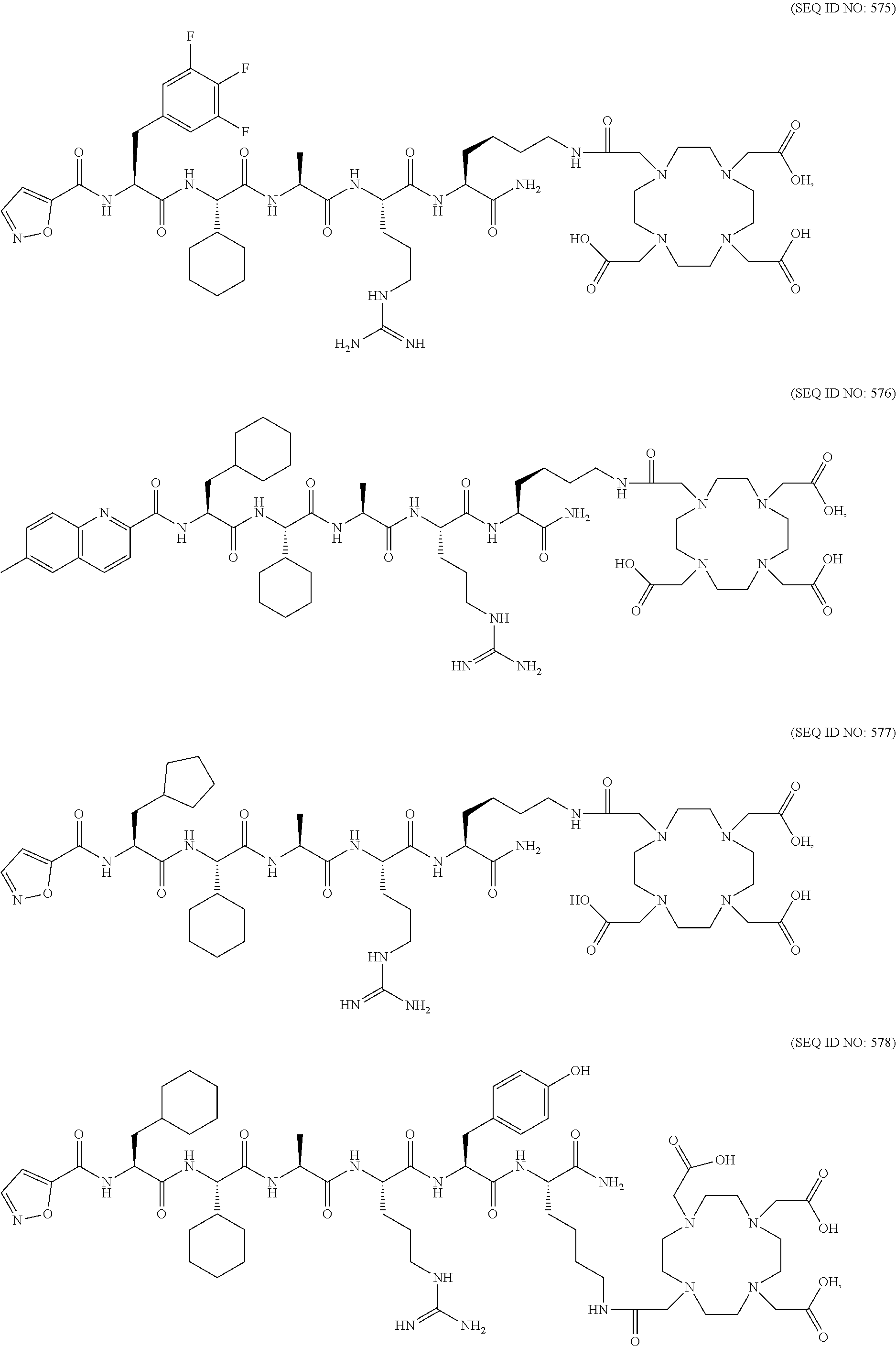

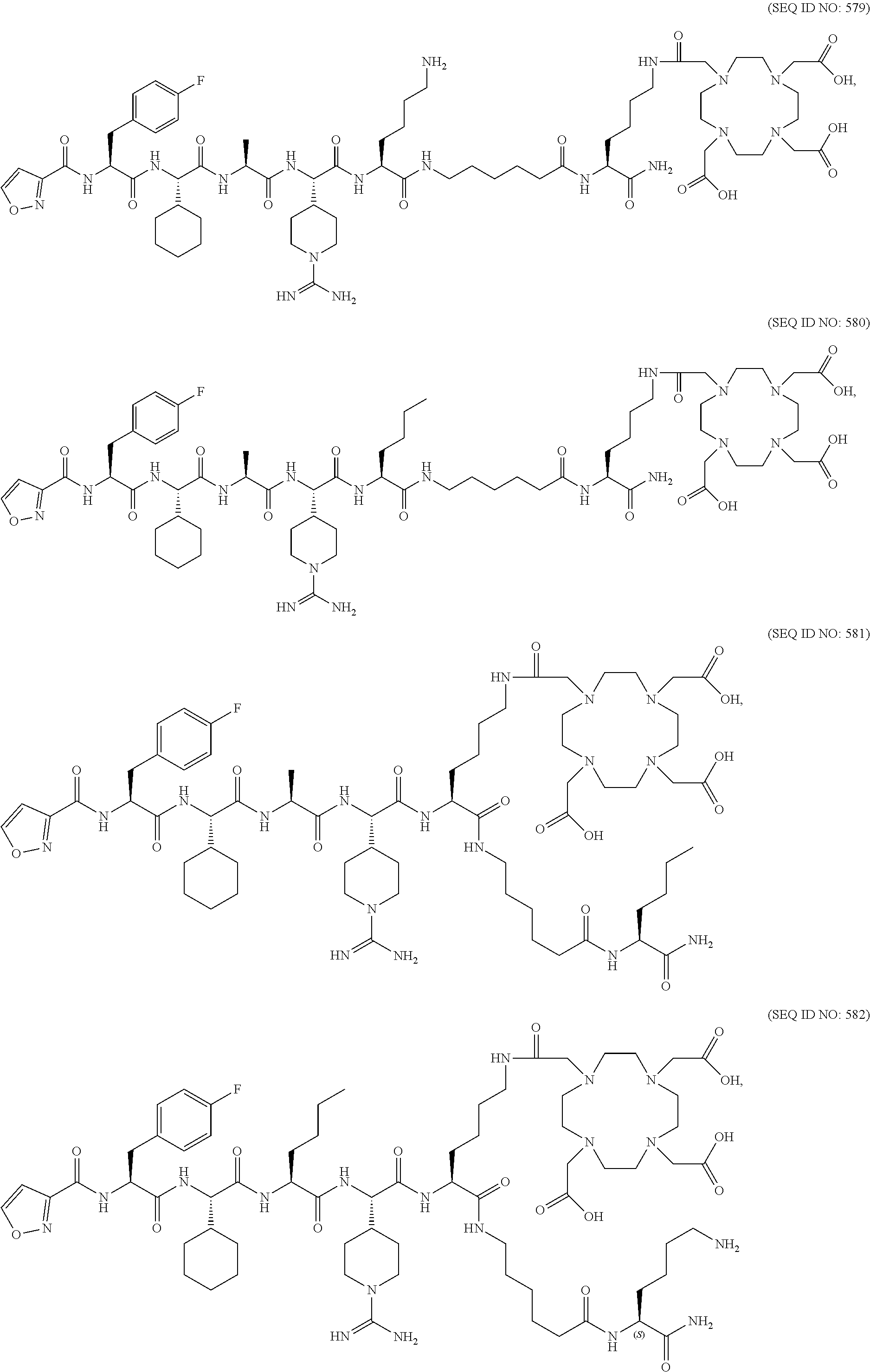

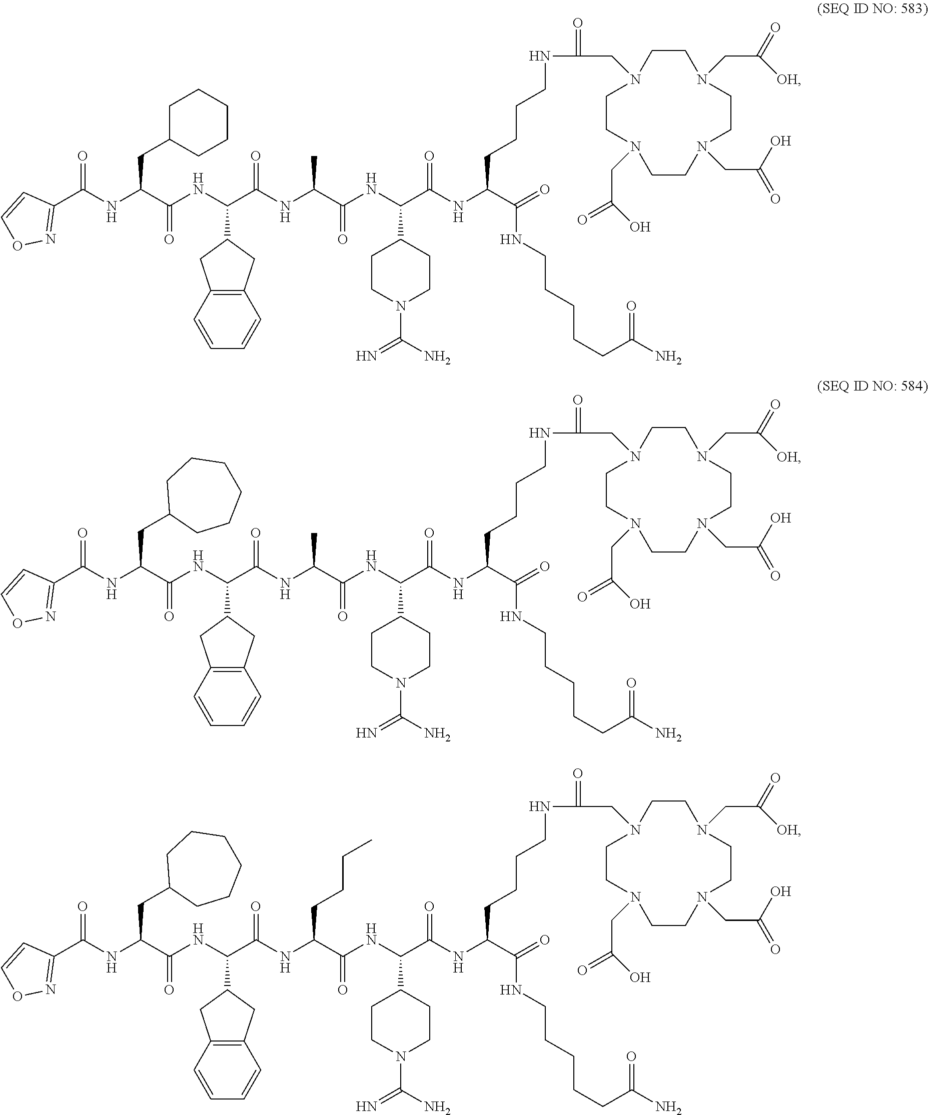

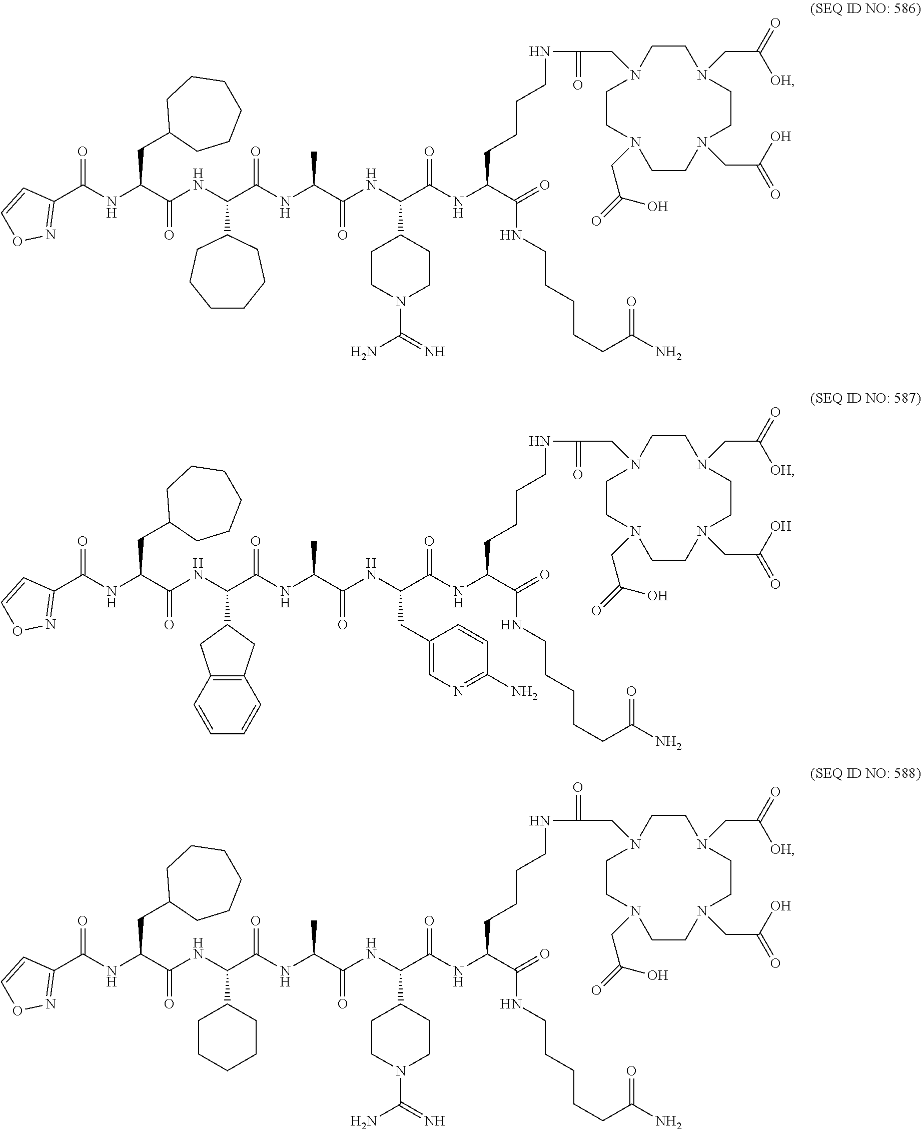

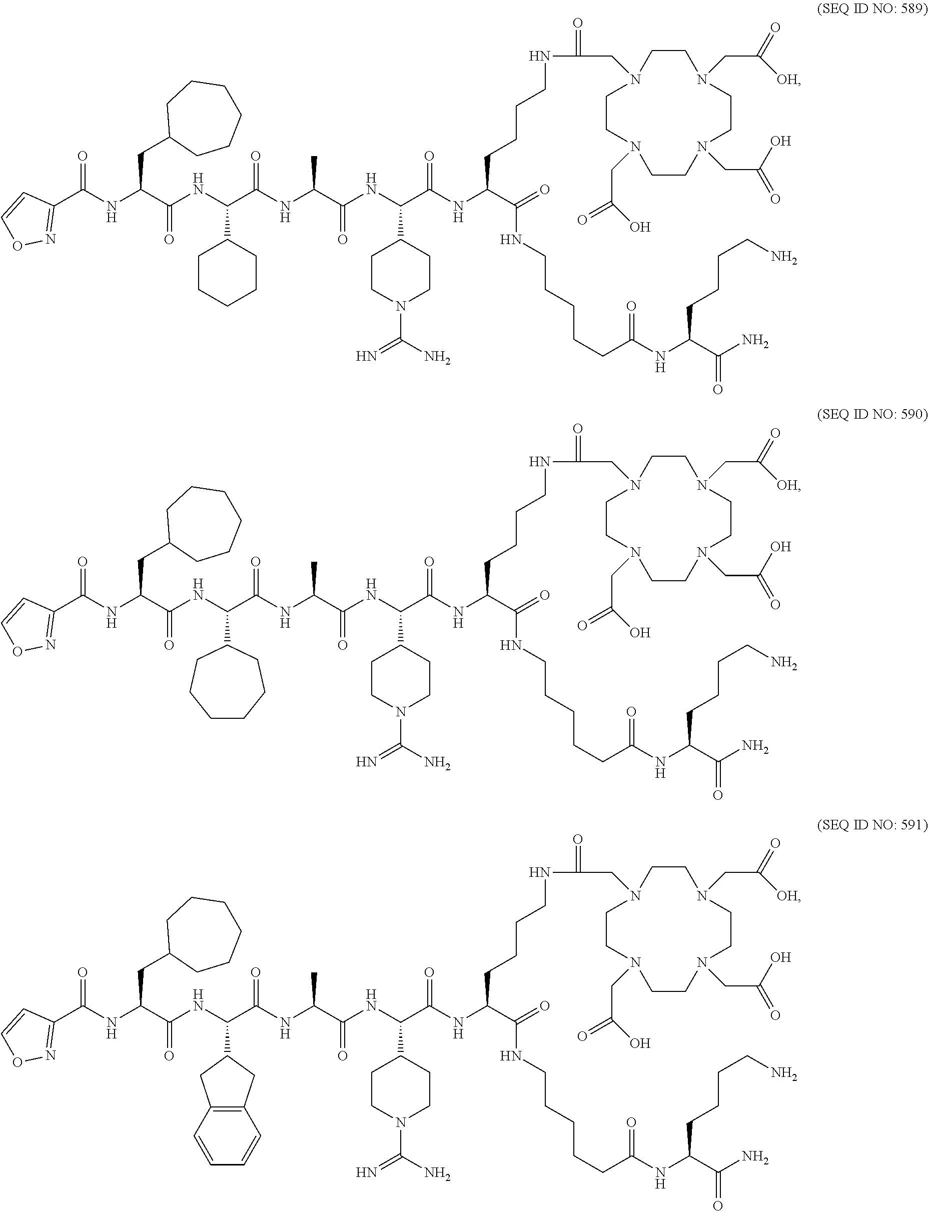

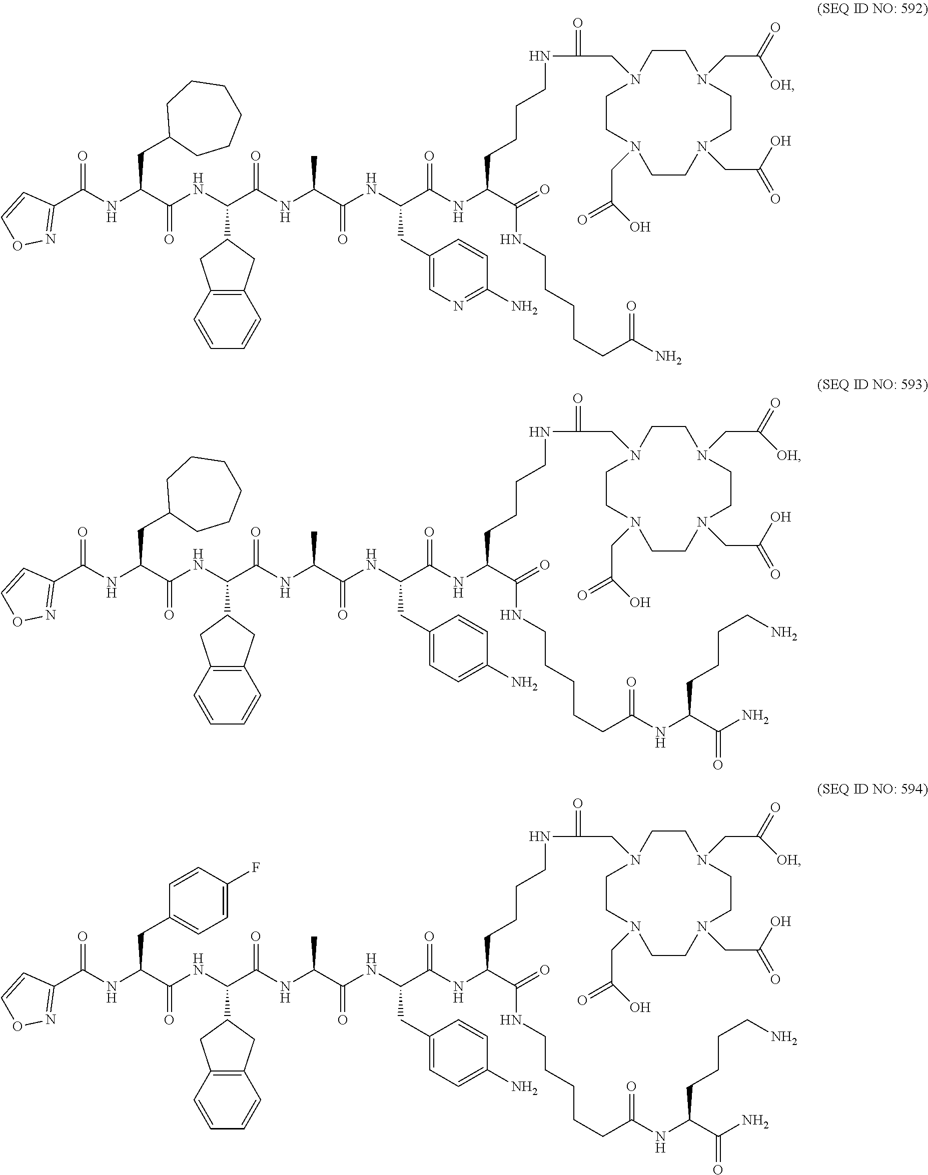

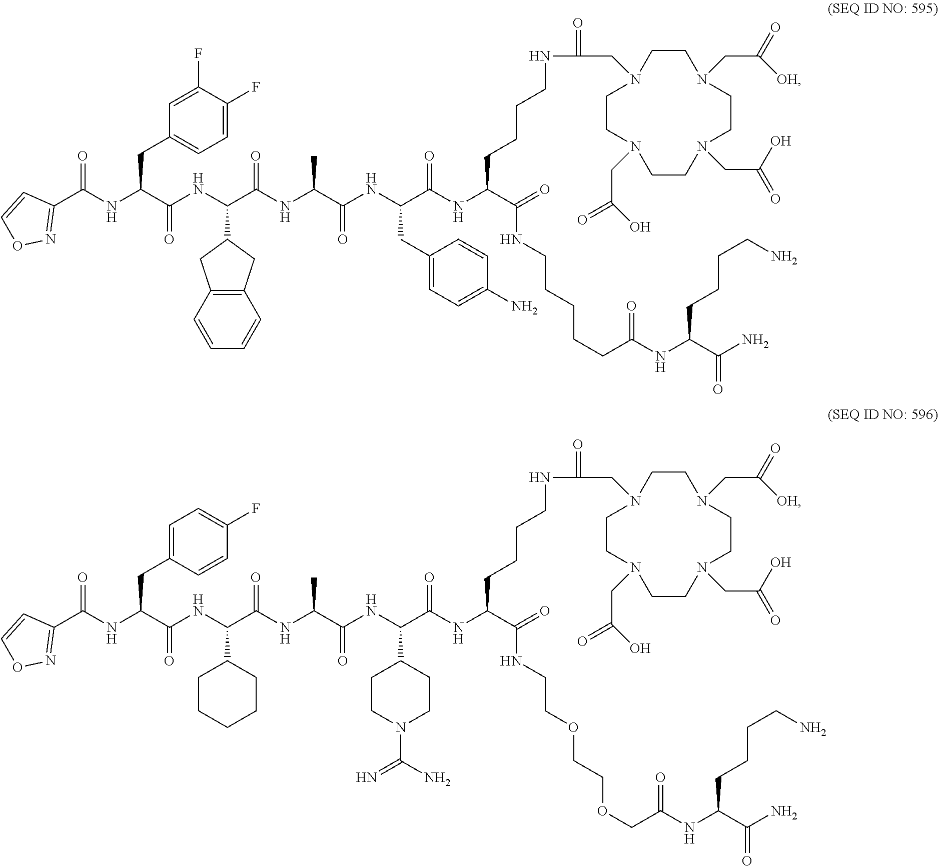 [SMILES: C[C@H](NC(=O)[C@@H](NC(=O)[C@H](Cc1ccc(F)c(F)c1)NC(=O)c1ccon1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O ; C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCN(C(=N)N)CC1]

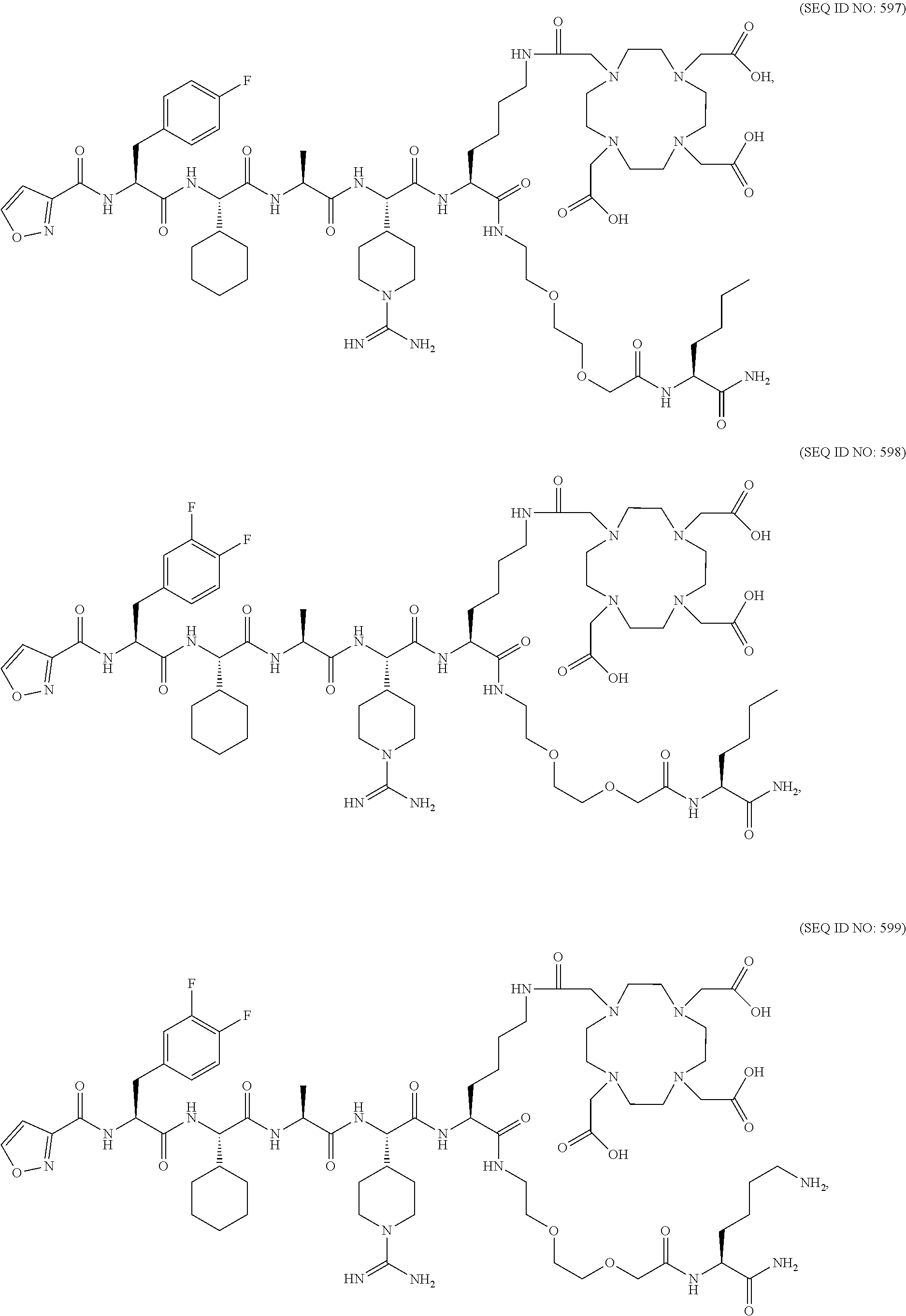

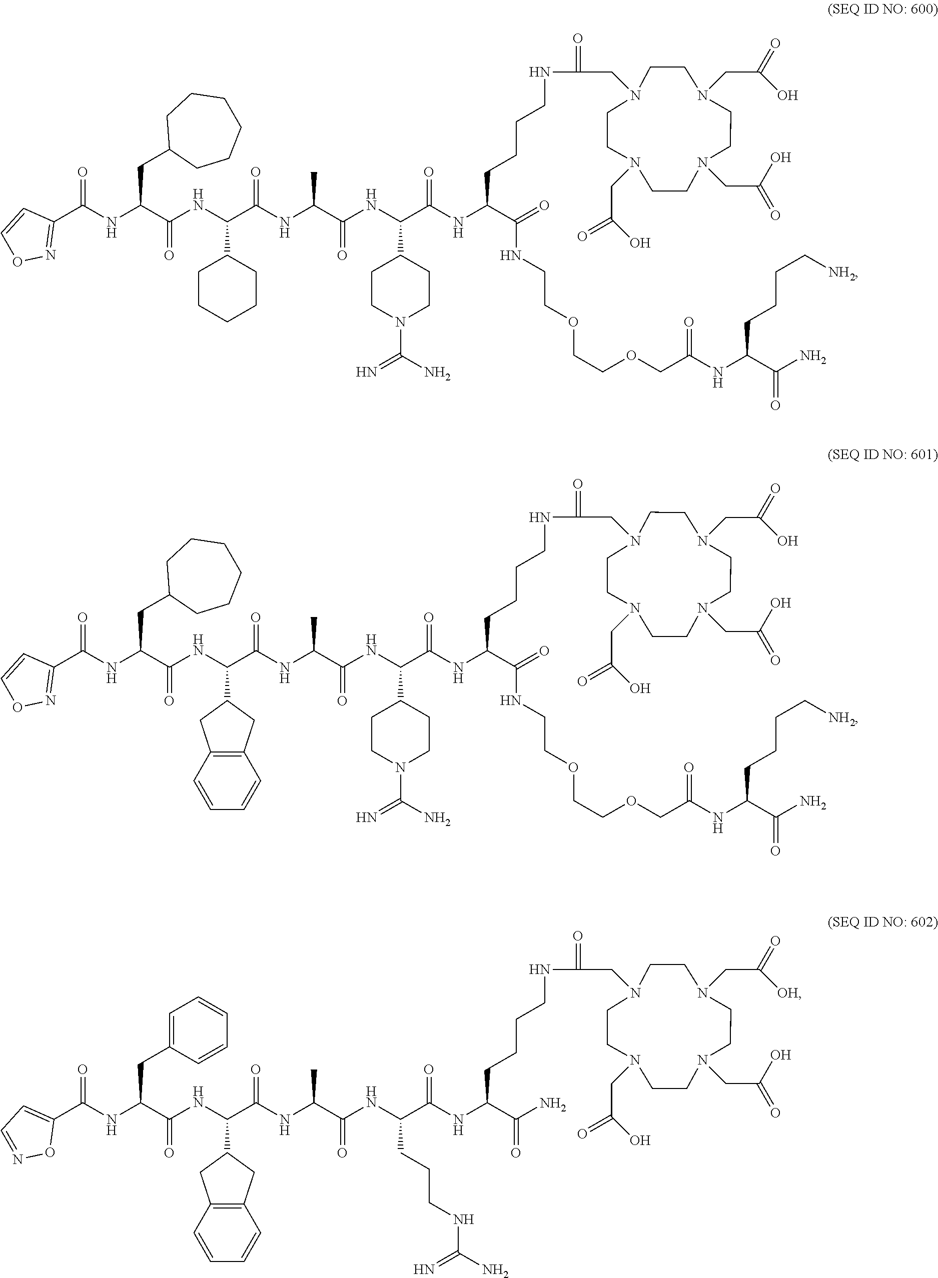

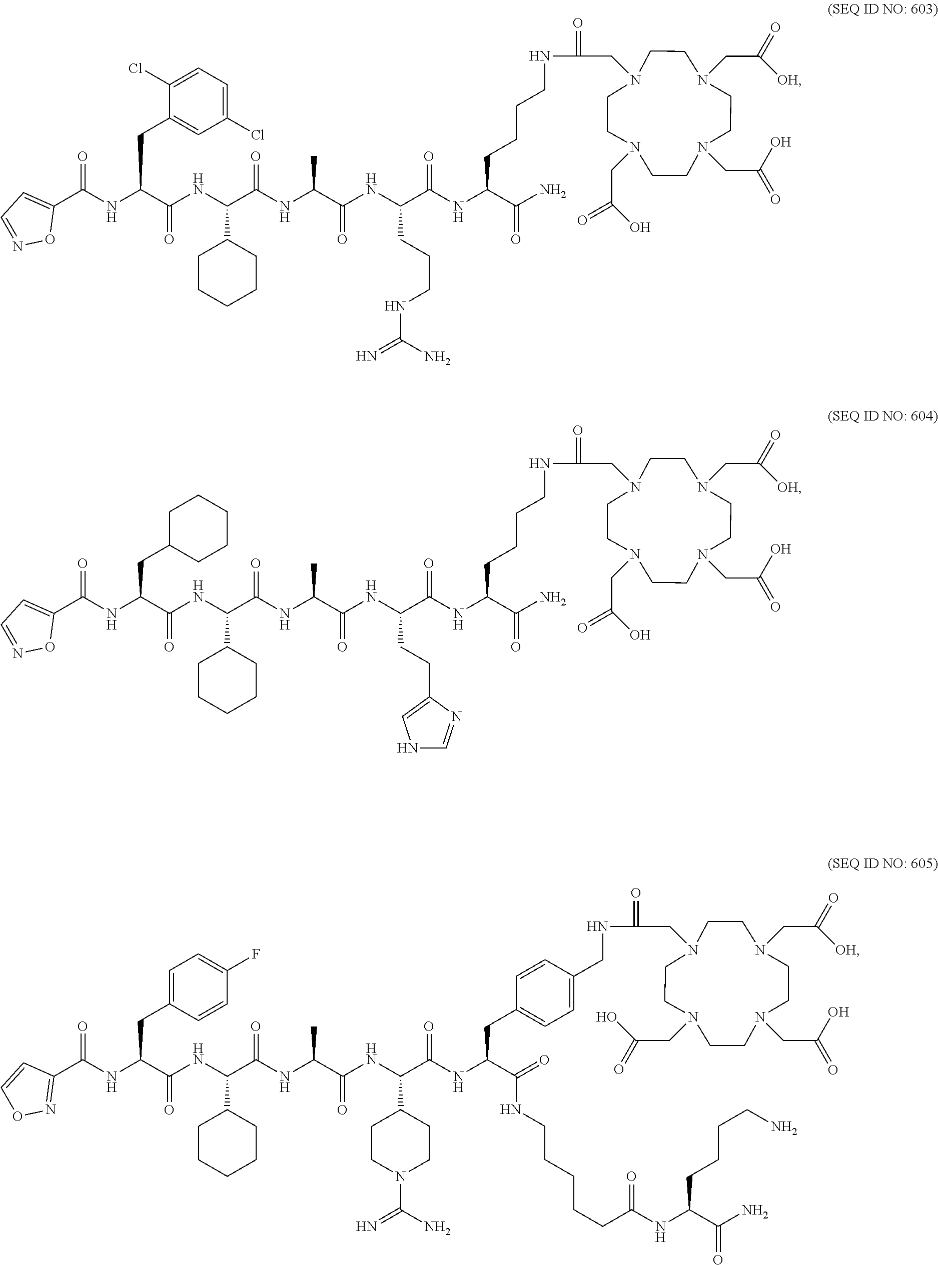

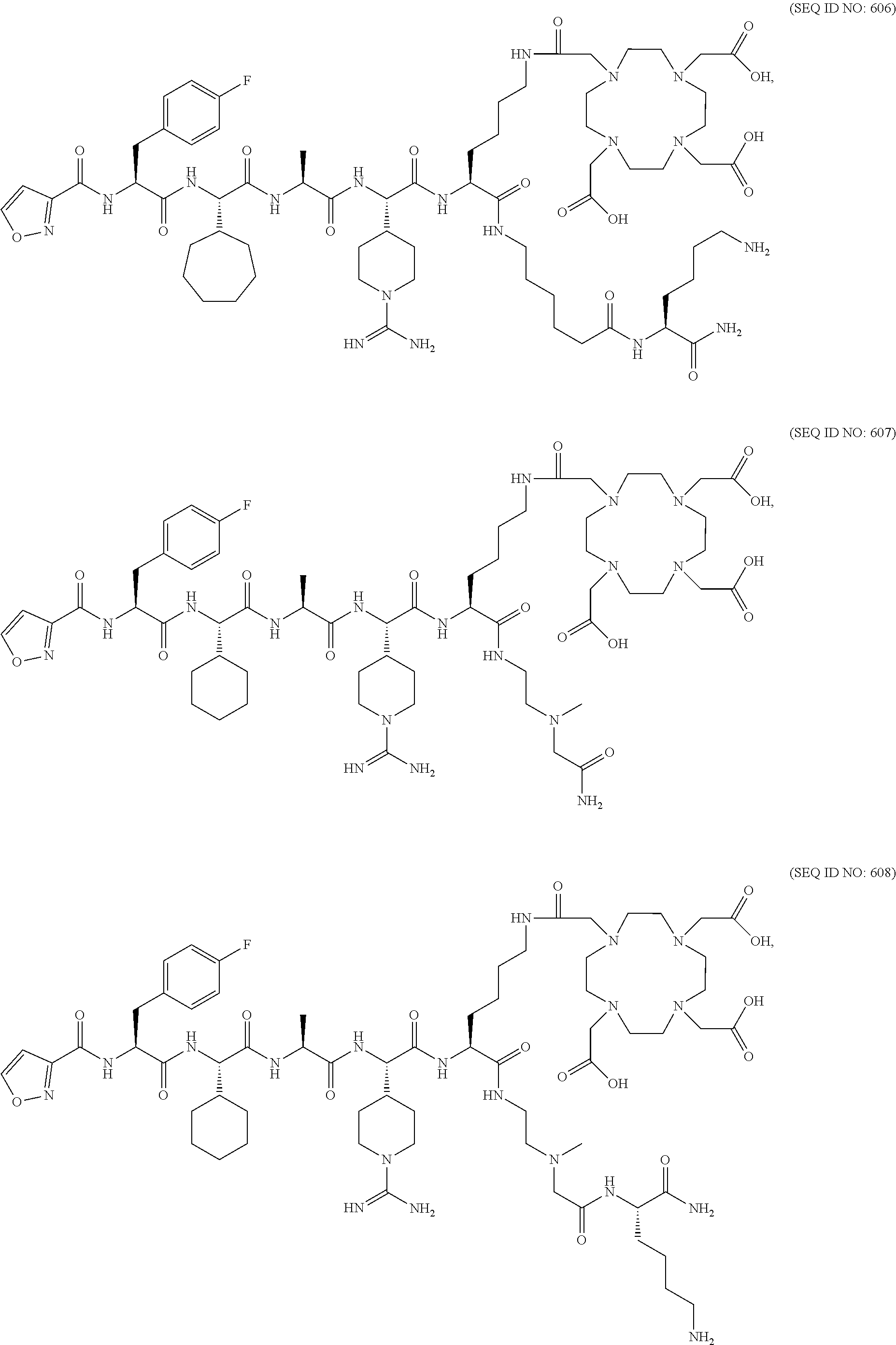

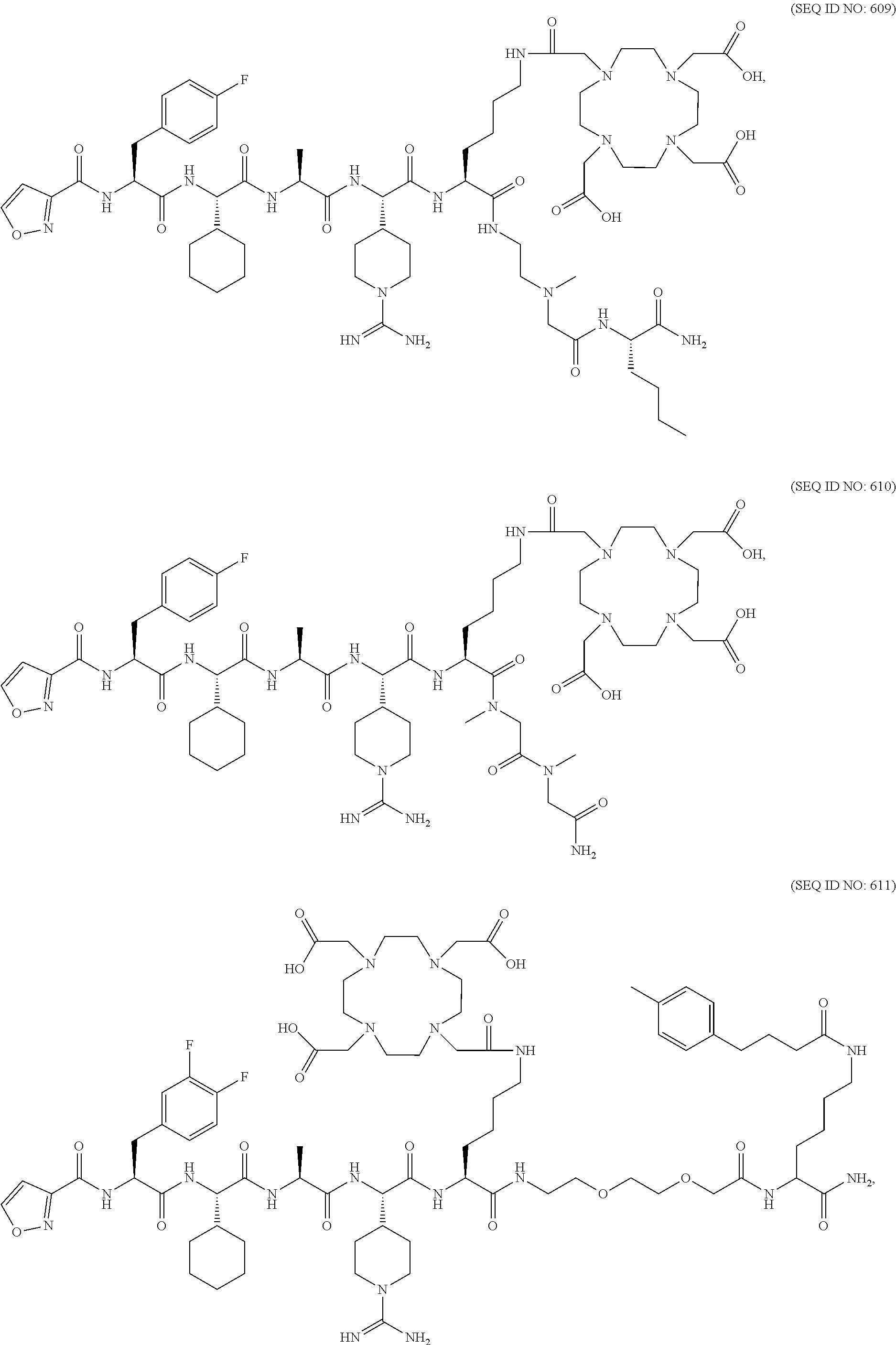

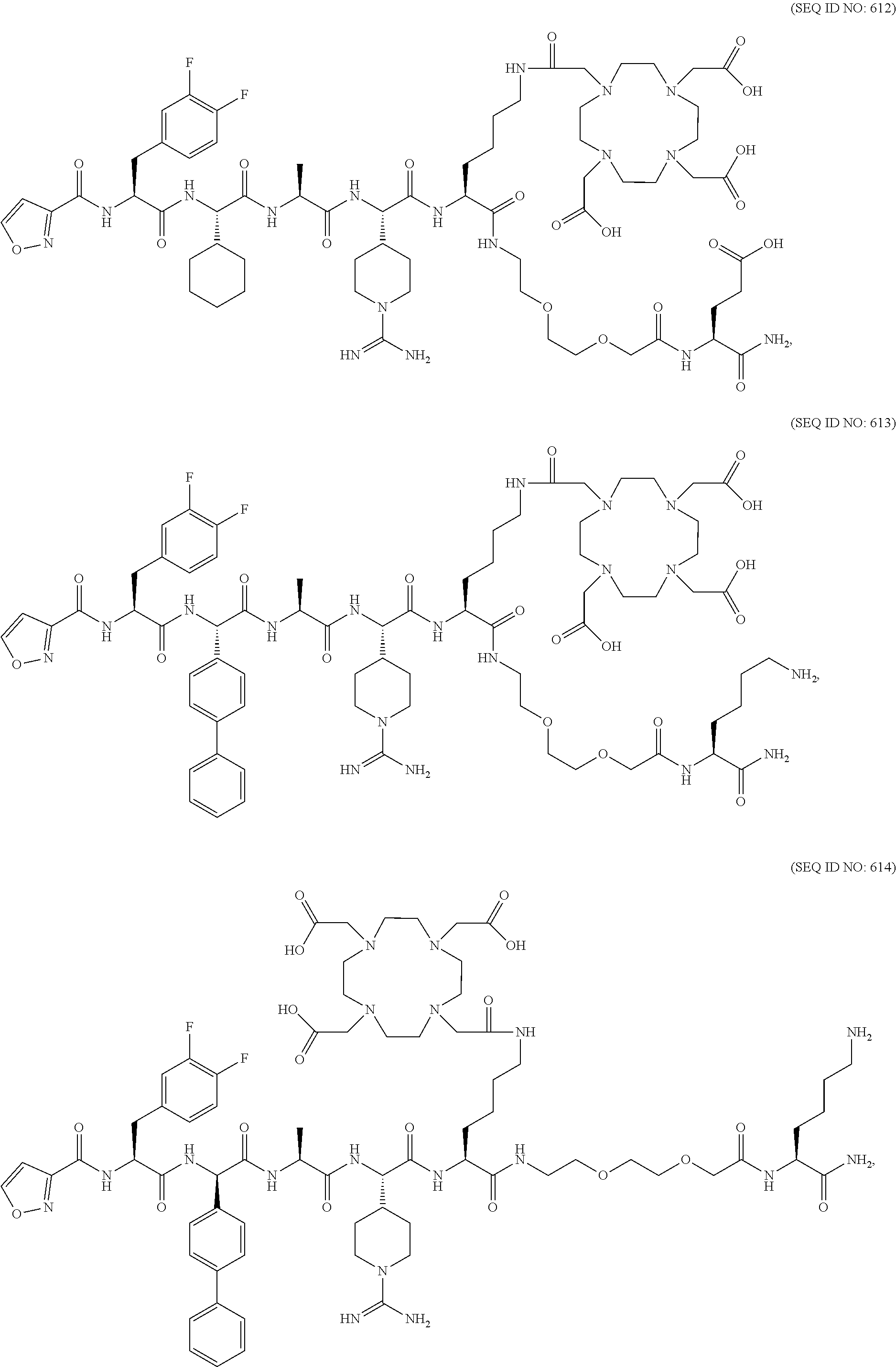

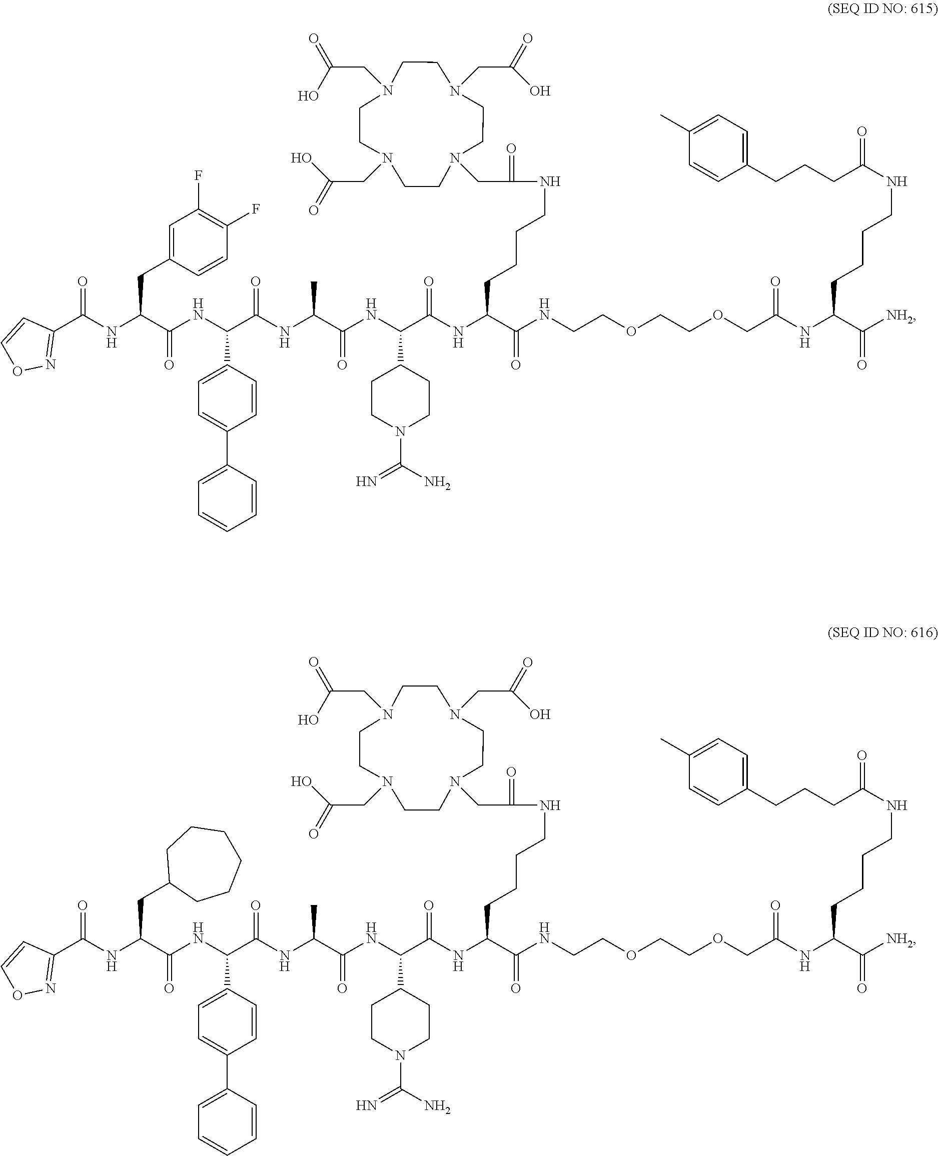 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)c2ccc(-c3ccccc3)cc2)C2CCN(C(=N)N)CC2)C(N)=O)cc1 ; Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](CC2CCCCCC2)NC(=O)c2ccon2)c2ccc(-c3ccccc3)cc2)C2CCN(C(=N)N)CC2)C(N)=O)cc1]

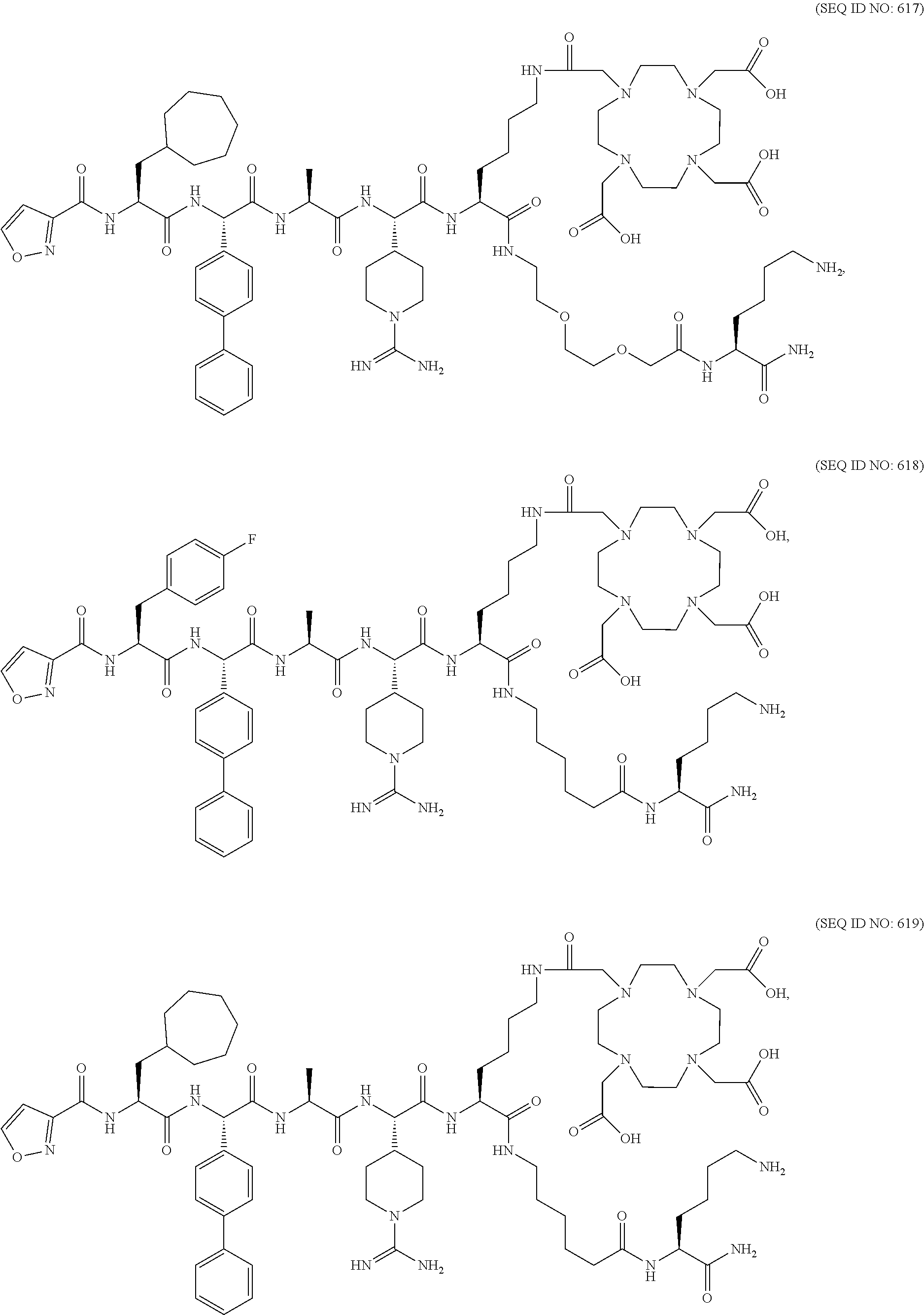

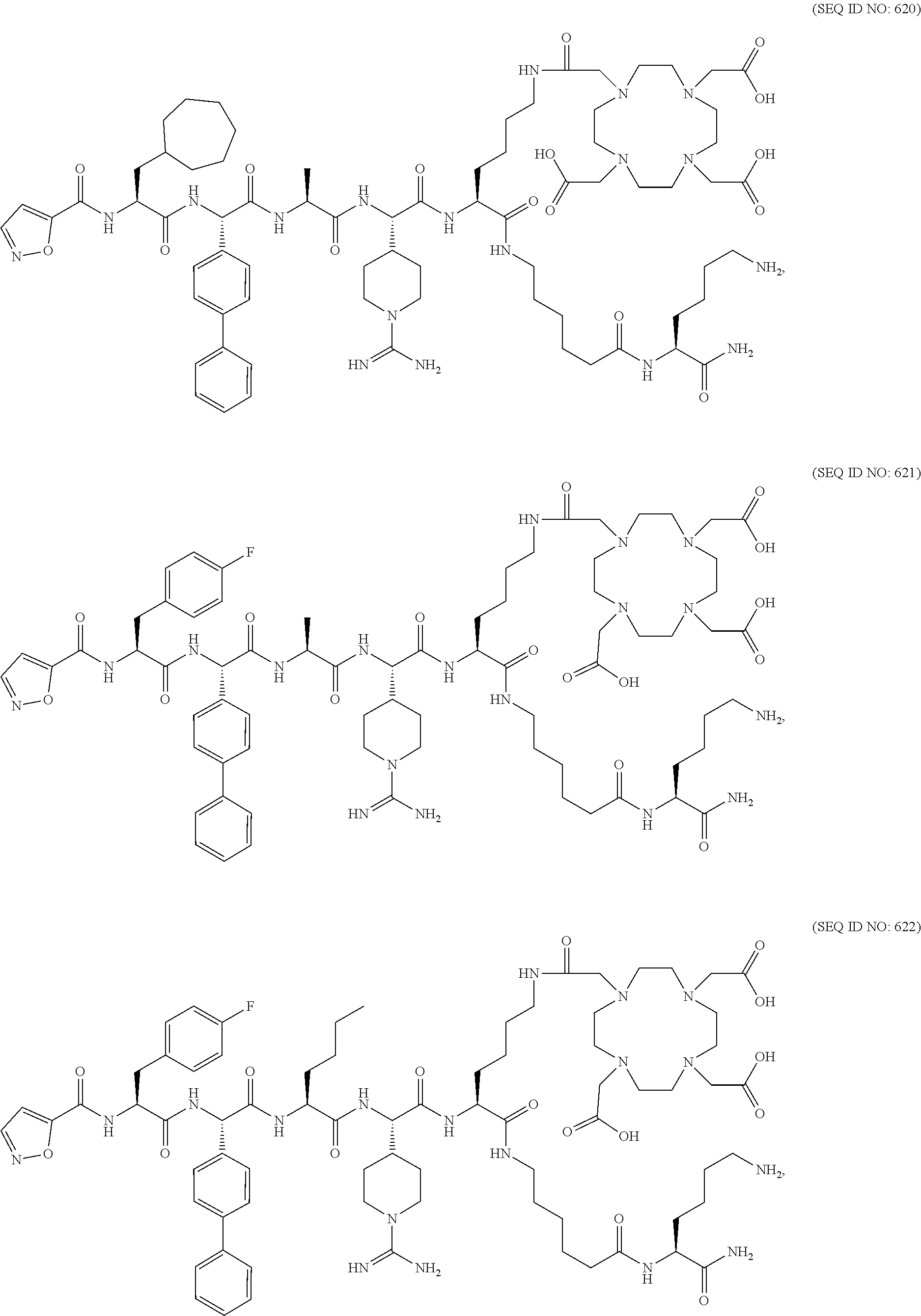

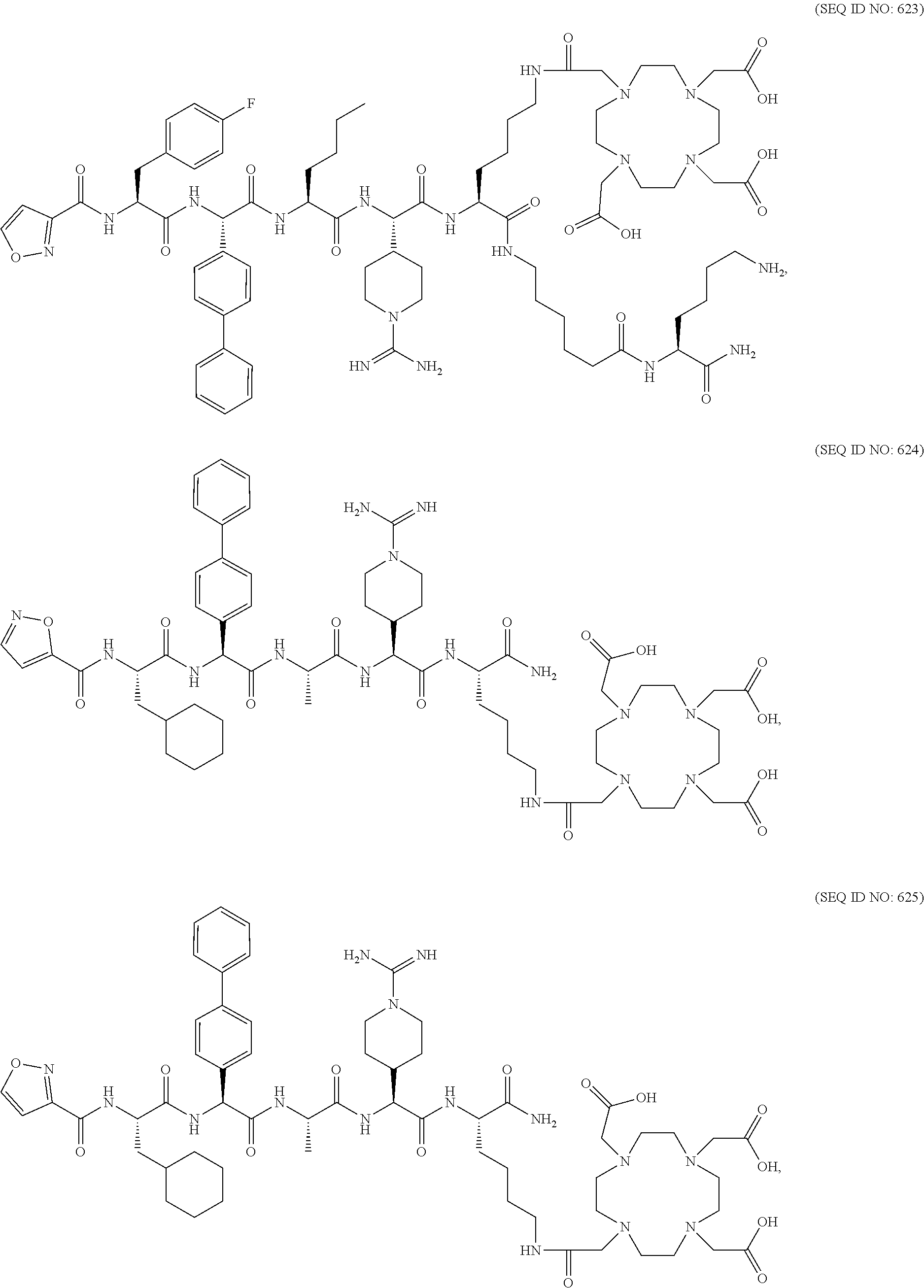

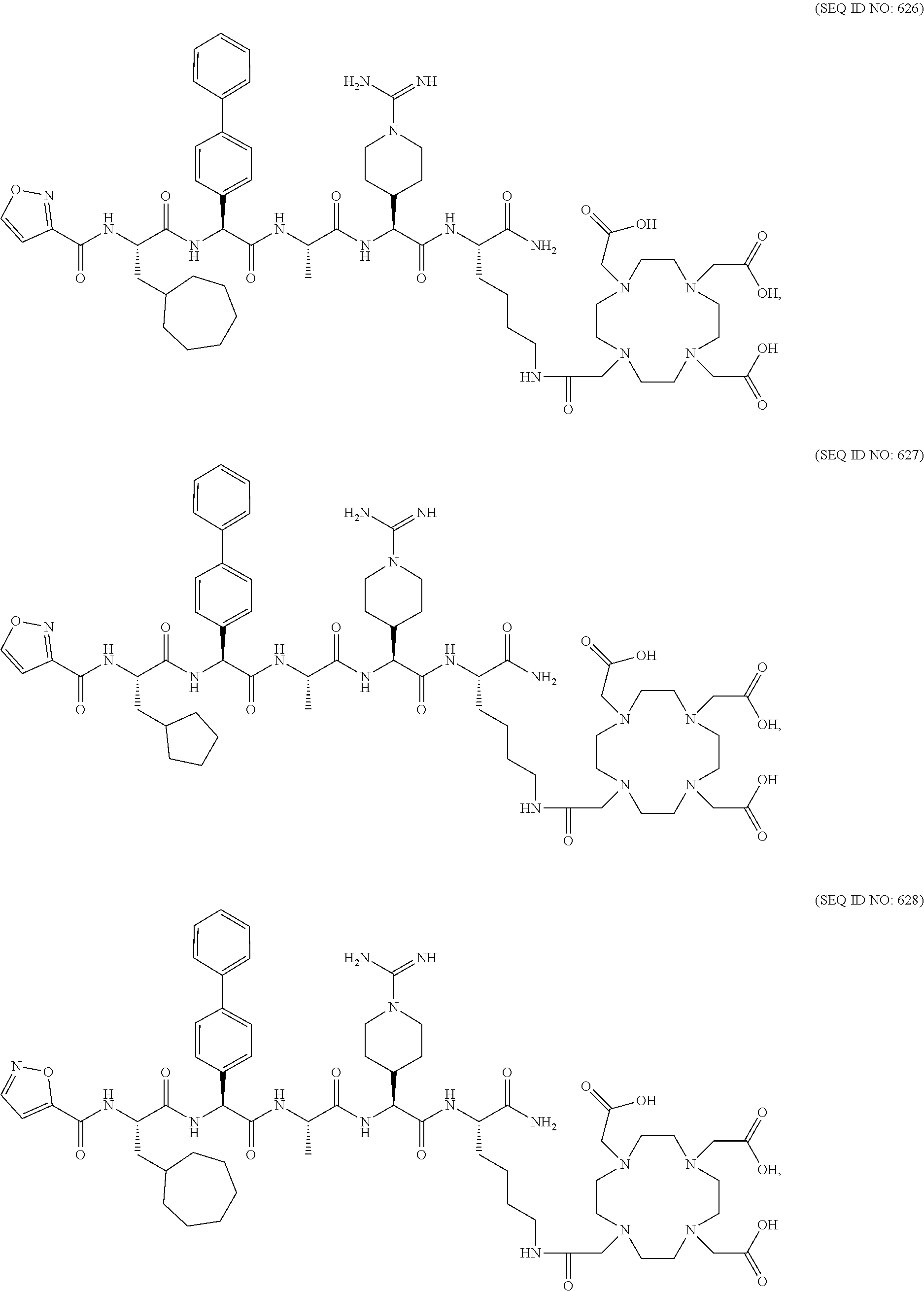

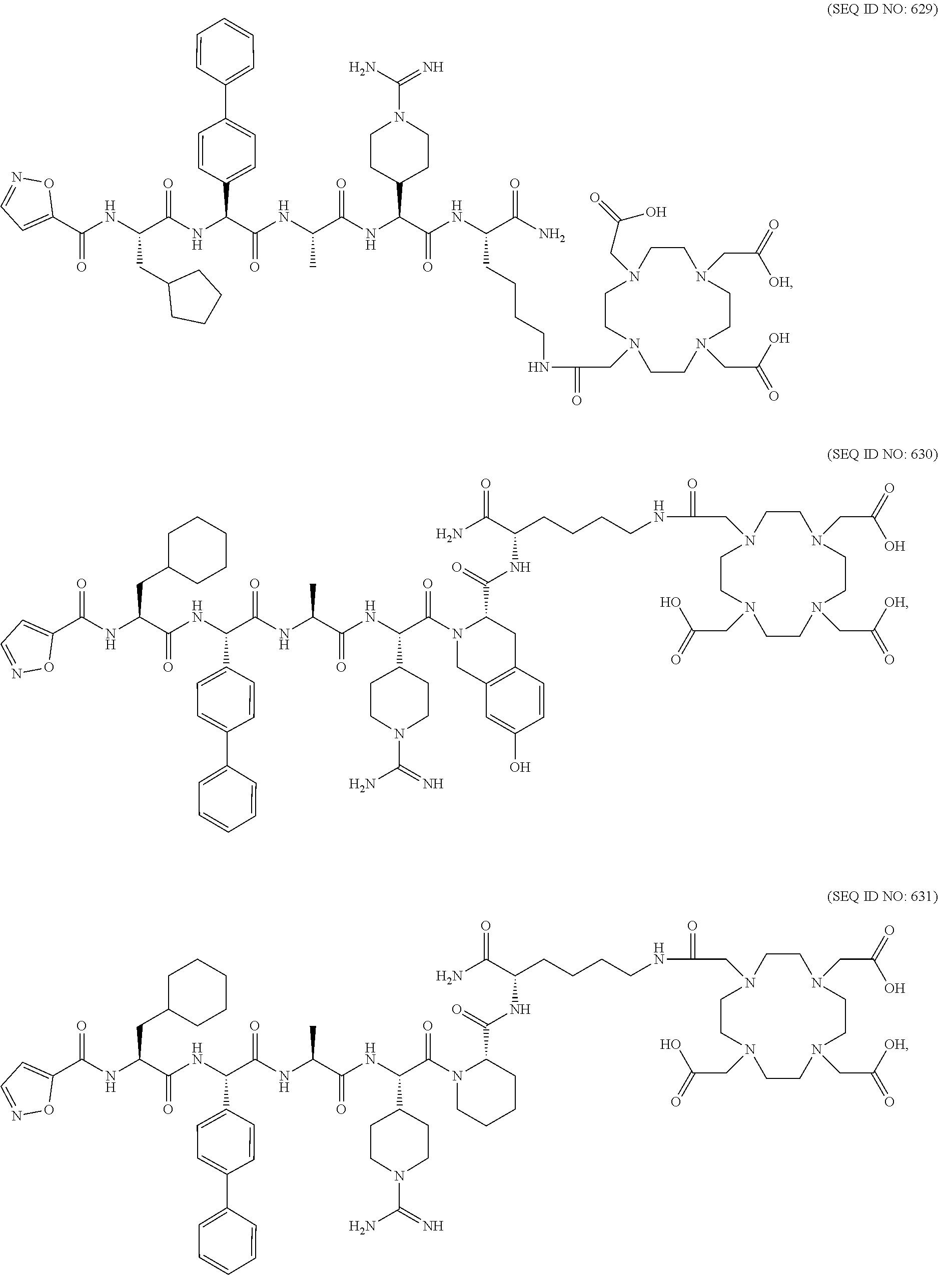

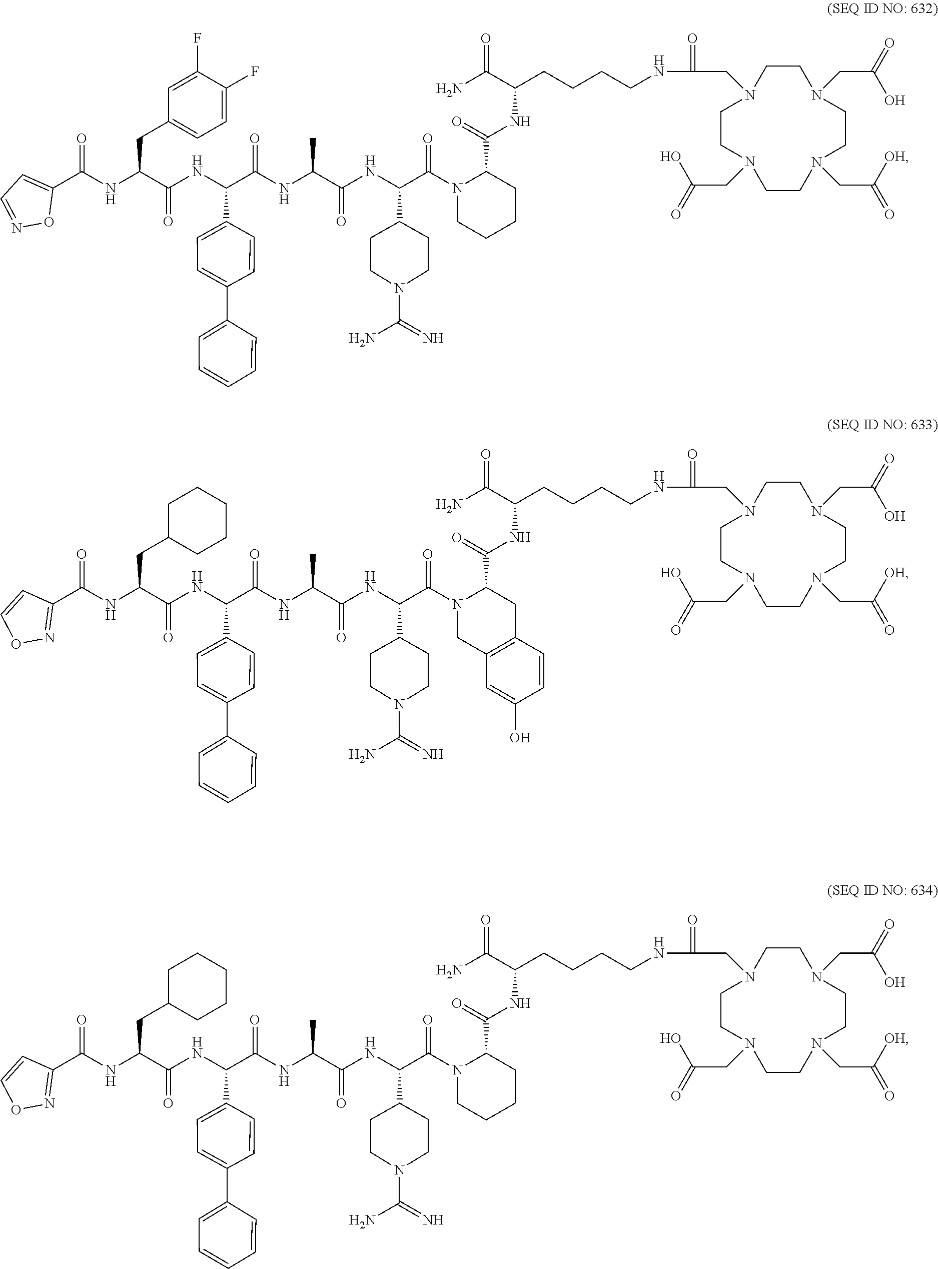

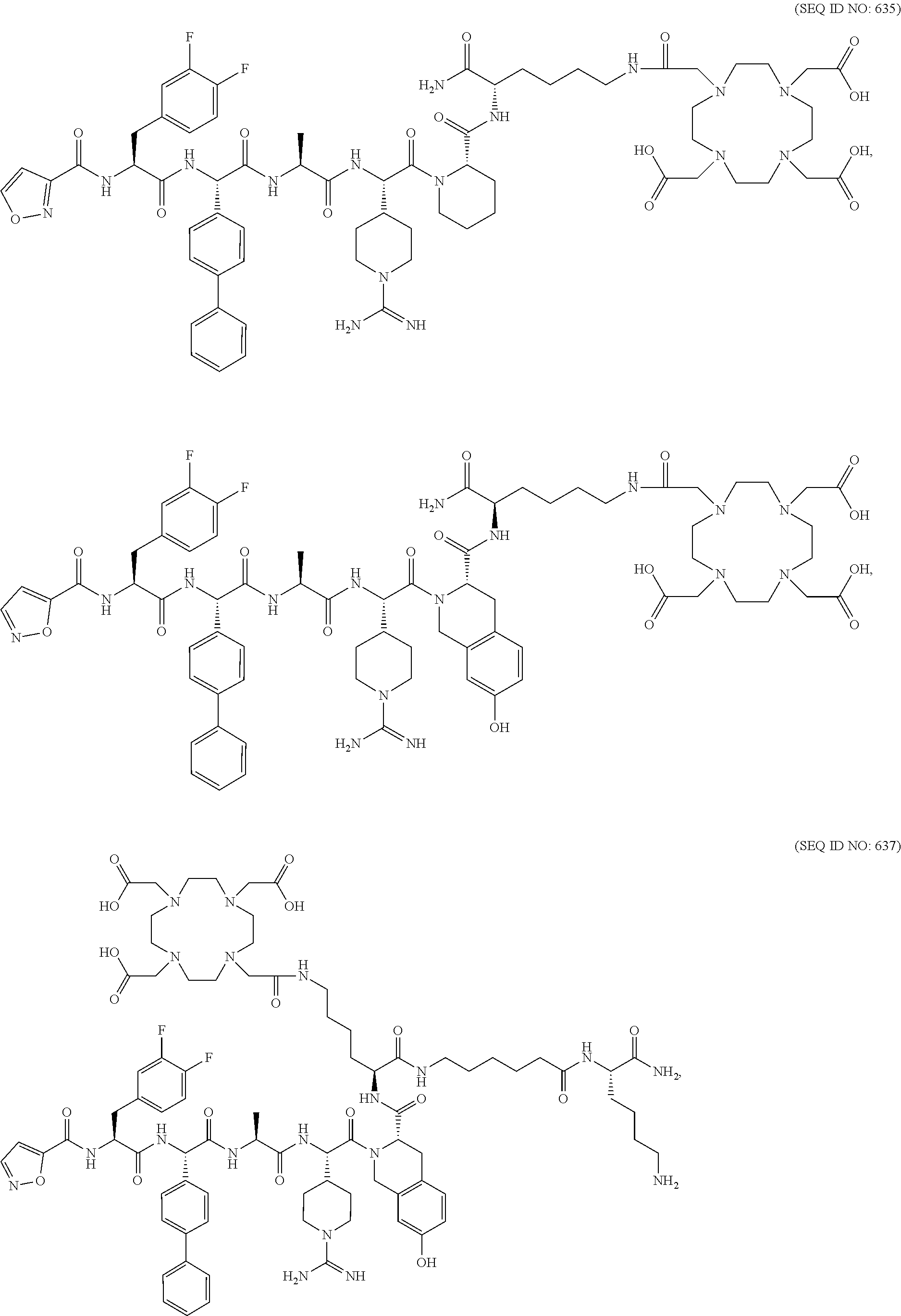

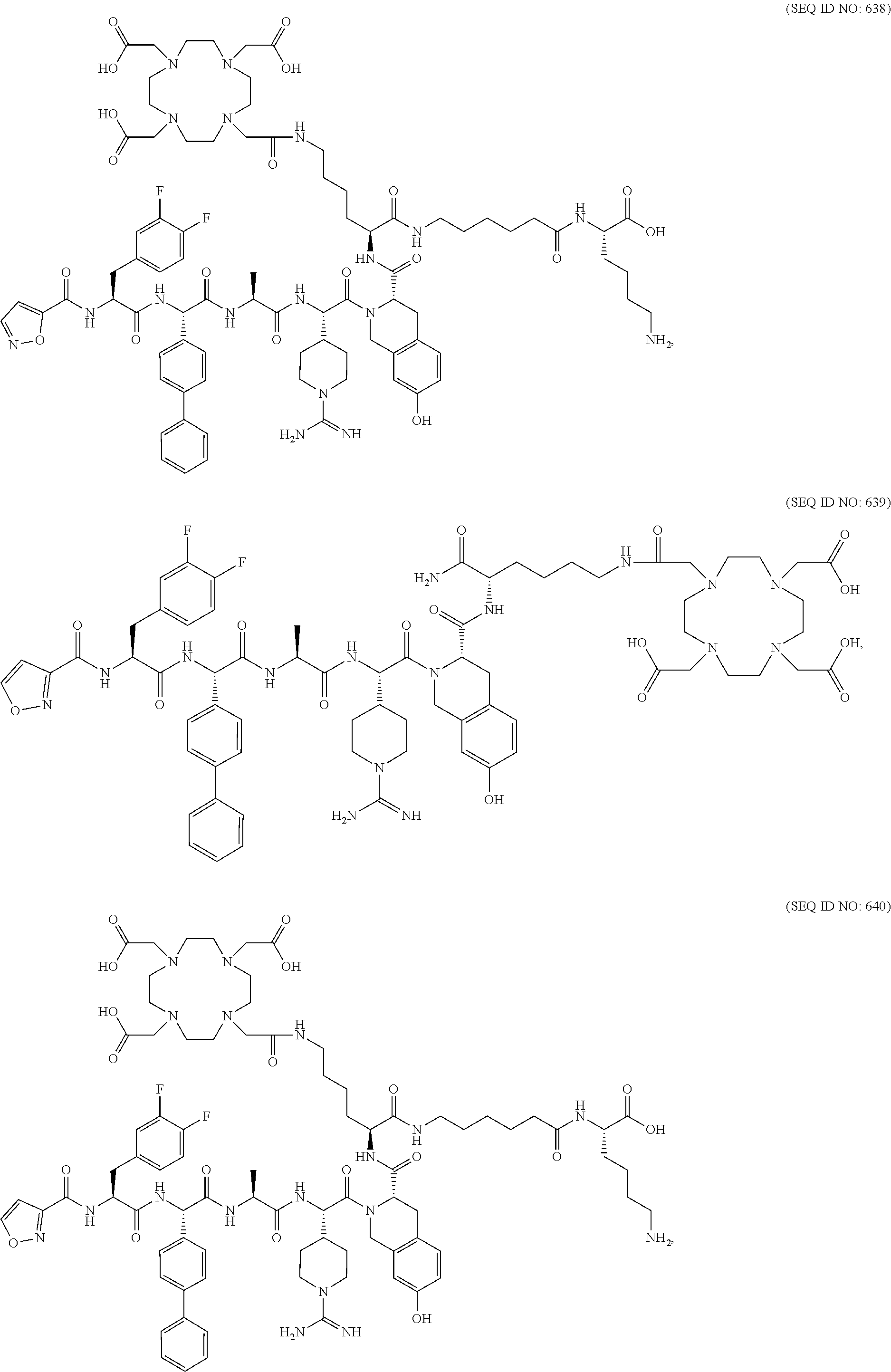

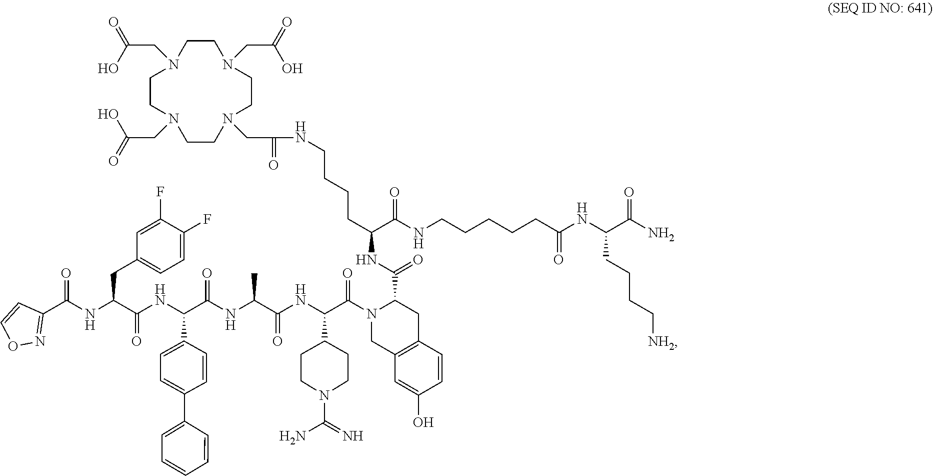 [SMILES: C[C@H](NC(=O)[C@@H](NC(=O)[C@H](Cc1ccc(F)c(F)c1)NC(=O)c1ccon1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

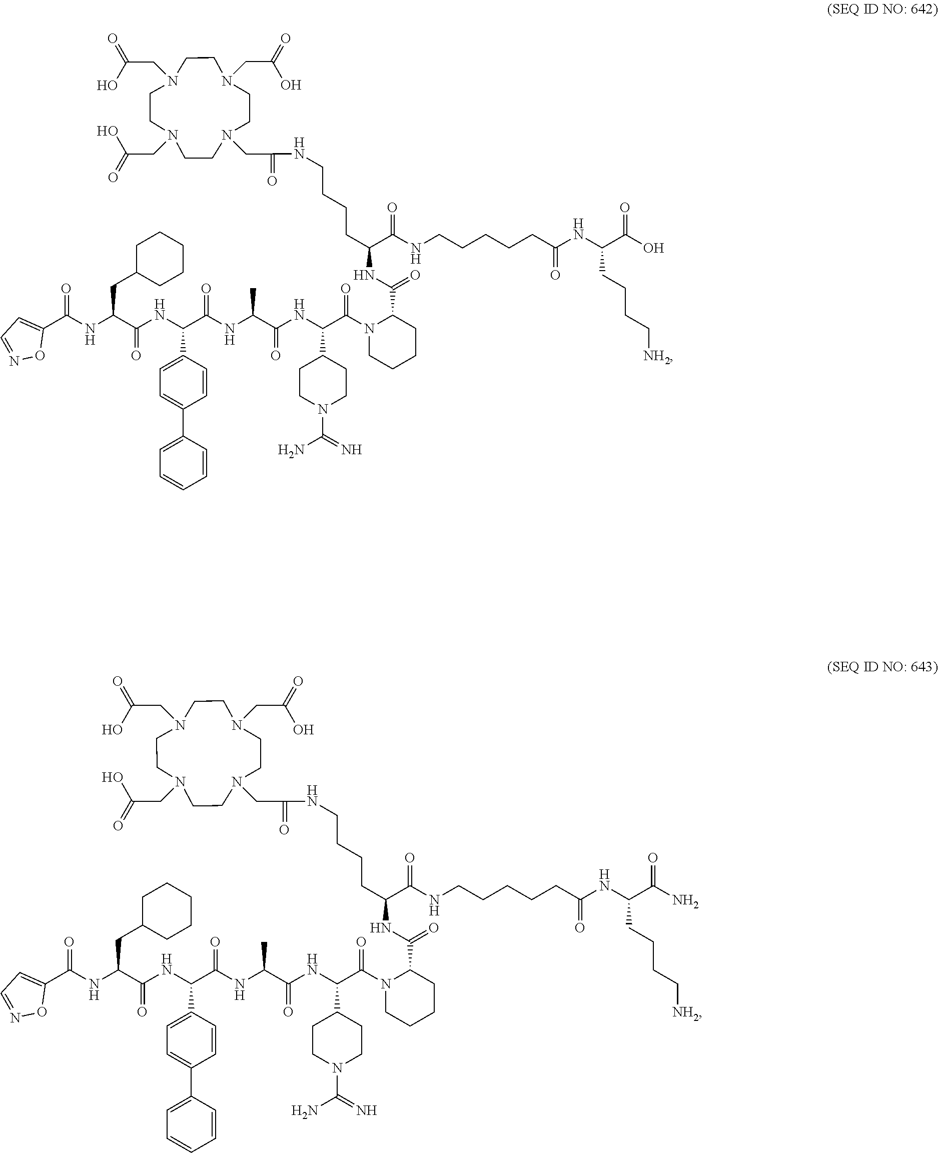 [SMILES: C[C@H](NC(=O)[C@@H](NC(=O)[C@H](CC1CCCCC1)NC(=O)c1ccno1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(=O)O)C1CCN(C(=N)N)CC1 ; C[C@H](NC(=O)[C@@H](NC(=O)[C@H](CC1CCCCC1)NC(=O)c1ccno1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

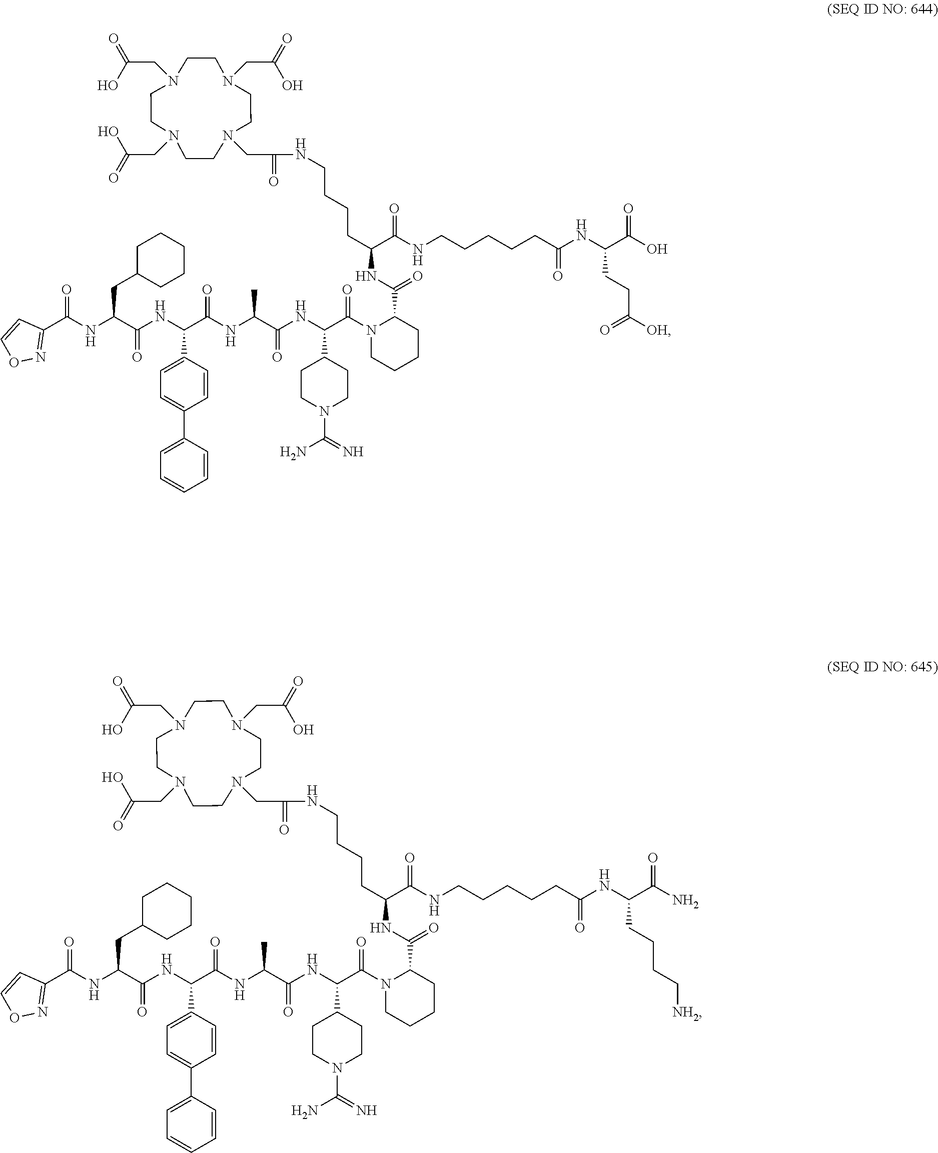 [SMILES: C[C@H](NC(=O)[C@@H](NC(=O)[C@H](CC1CCCCC1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCC(=O)O)C(=O)O)C1CCN(C(=N)N)CC1 ; C[C@H](NC(=O)[C@@H](NC(=O)[C@H](CC1CCCCC1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

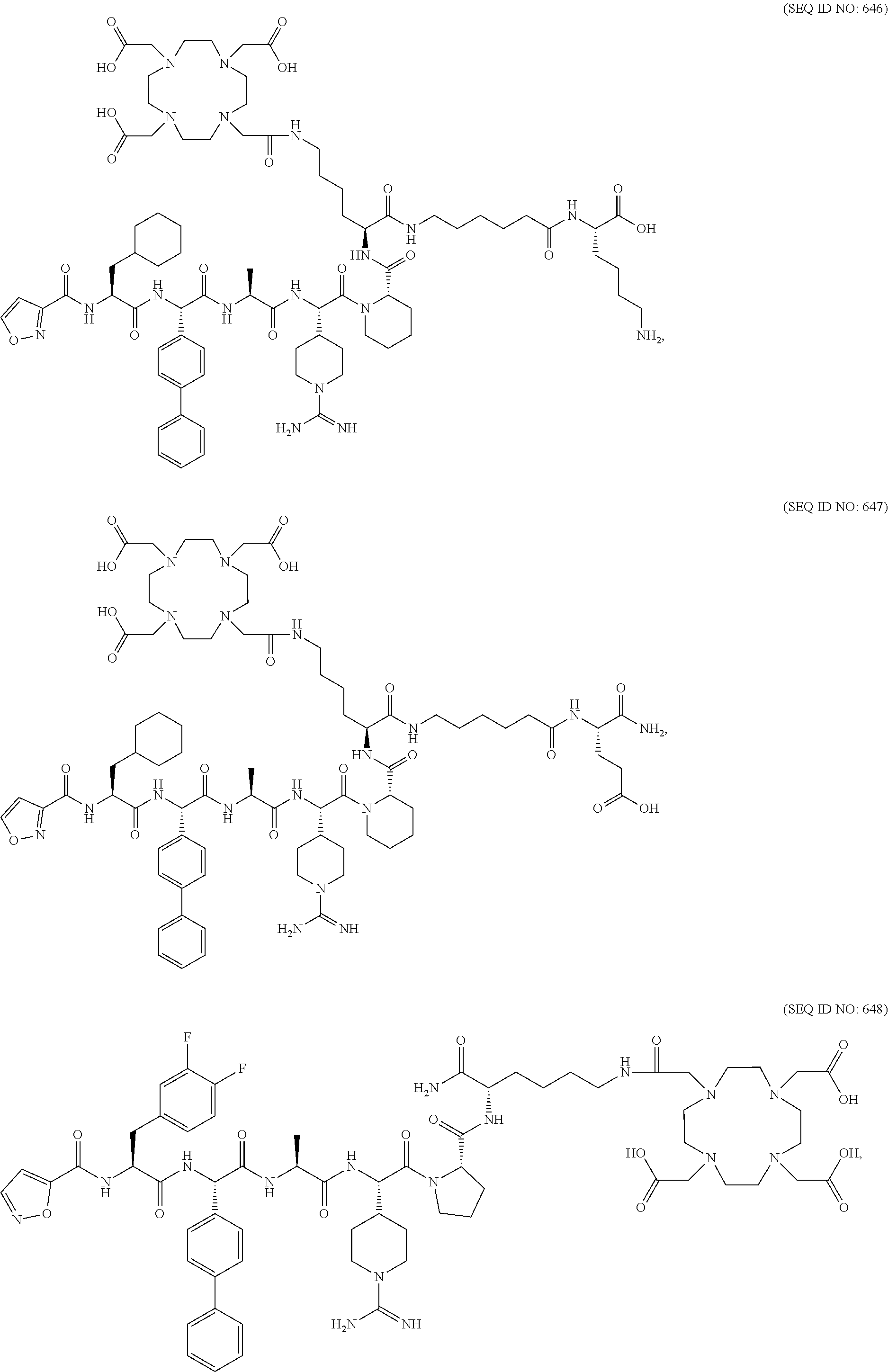

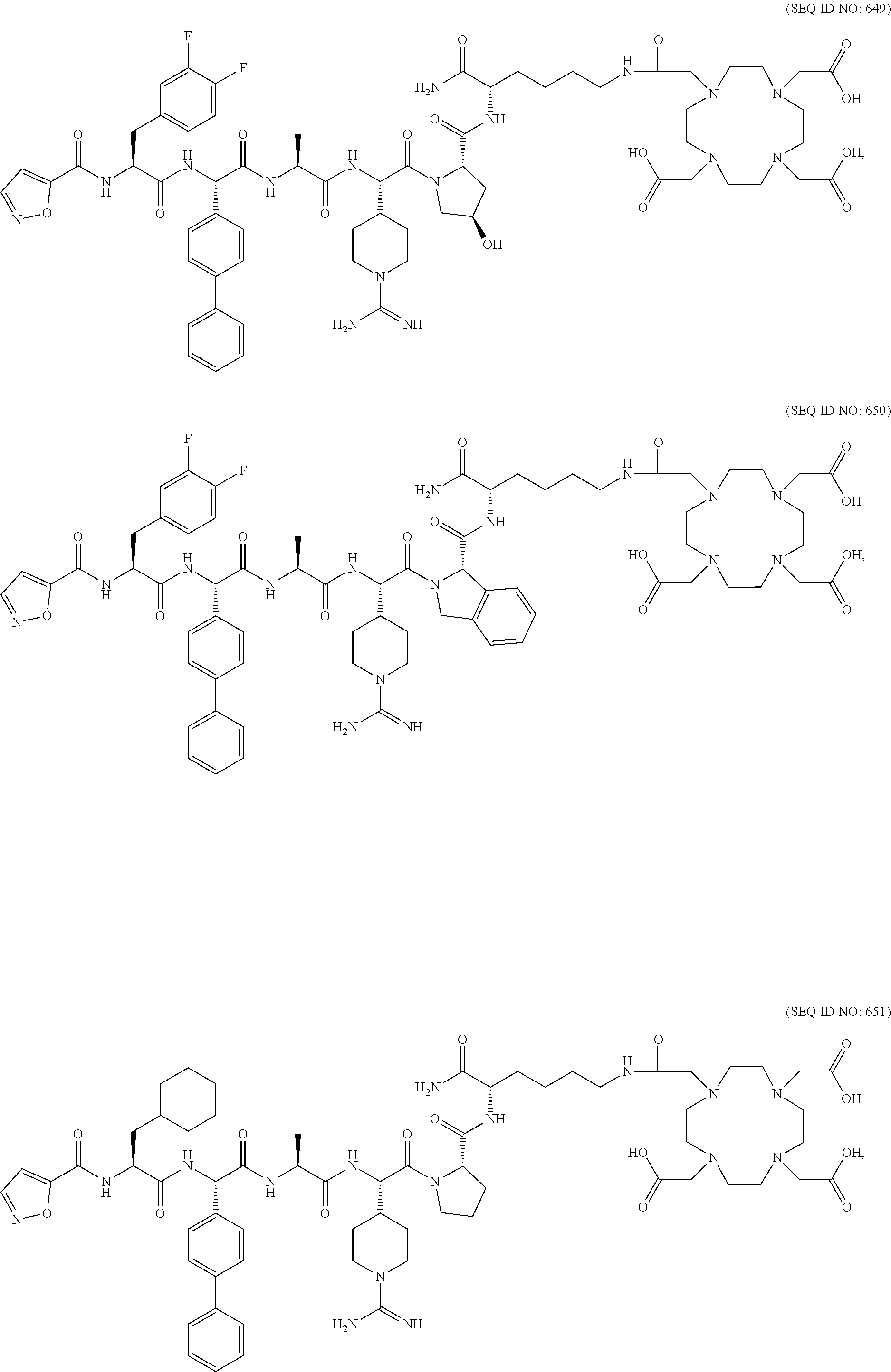

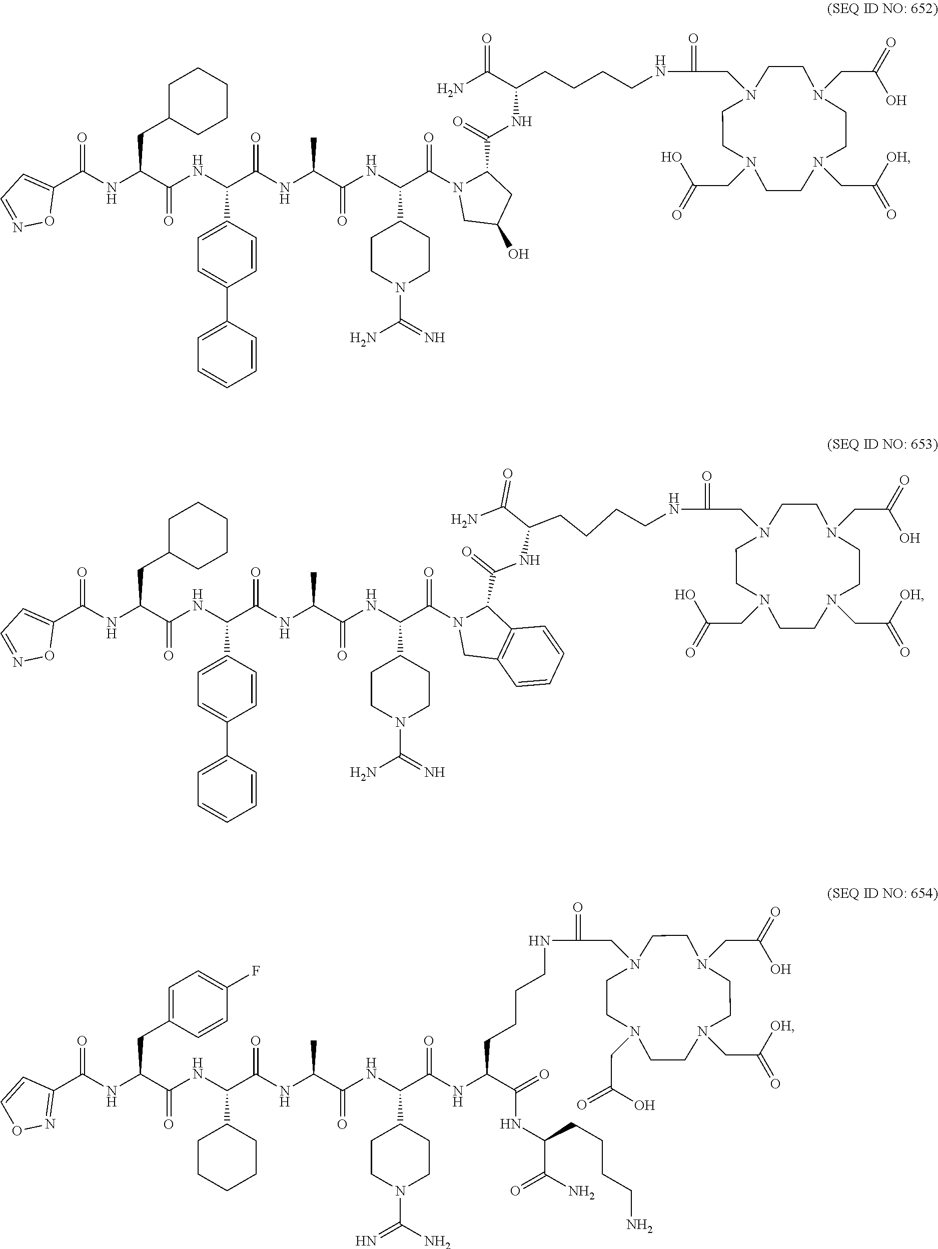

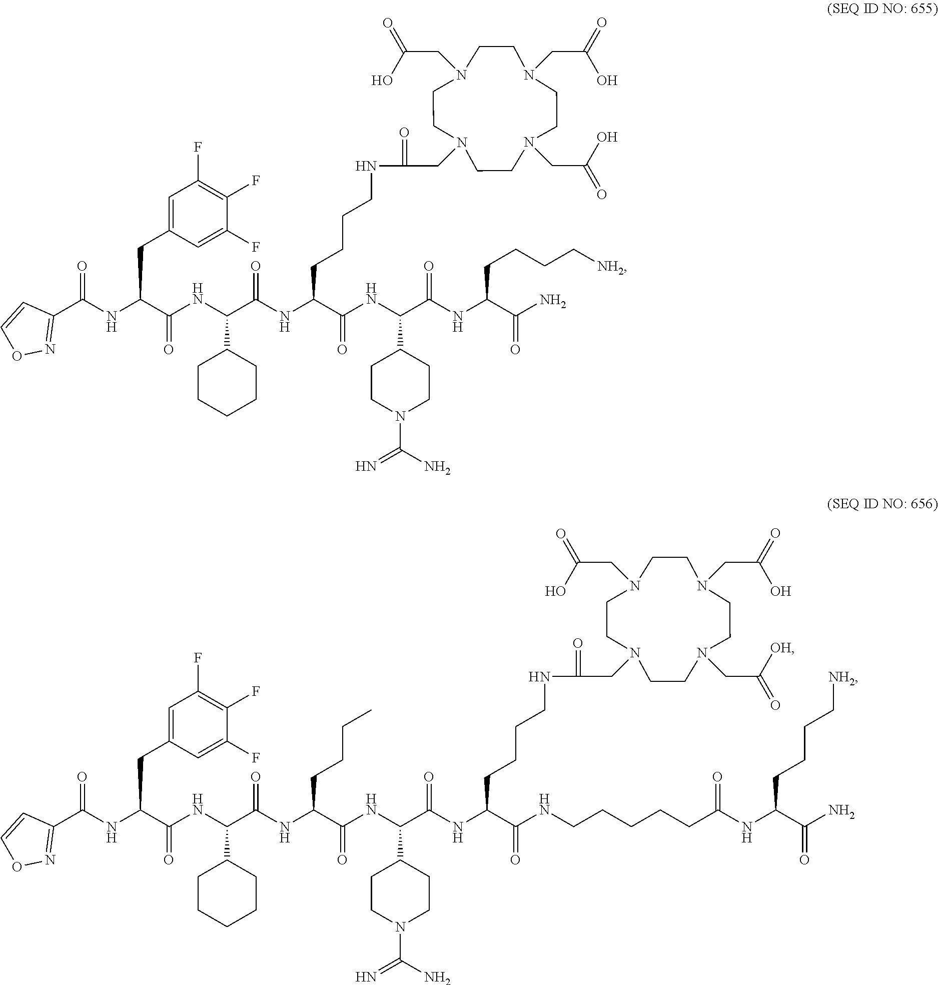 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@H](Cc2cc(F)c(F)c(F)c2)NC(=O)c2ccon2)C2CCCCC2)C(=O)N[C@@H](CCCCN)C(N)=O)CC1 ; CCCC[C@H](NC(=O)[C@@H](NC(=O)[C@H](Cc1cc(F)c(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

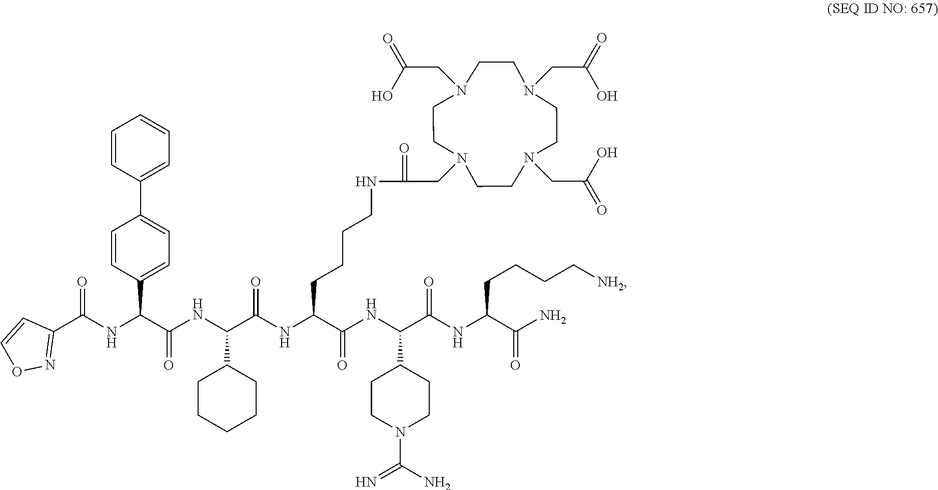 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@@H](NC(=O)c2ccon2)c2ccc(-c3ccccc3)cc2)C2CCCCC2)C(=O)N[C@@H](CCCCN)C(N)=O)CC1]

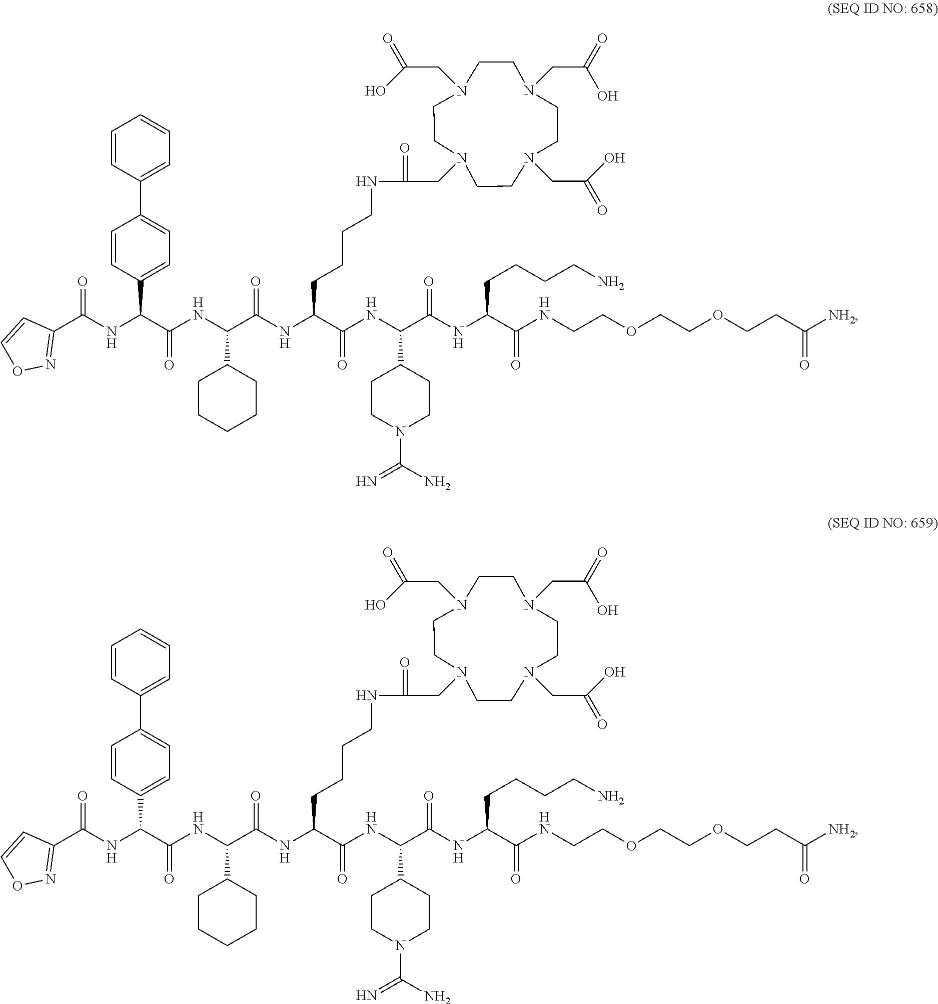 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@@H](NC(=O)c2ccon2)c2ccc(-c3ccccc3)cc2)C2CCCCC2)C(=O)N[C@@H](CCCCN)C(=O)NCCOCCOCCC(N)=O)CC1 ; N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@H](NC(=O)c2ccon2)c2ccc(-c3ccccc3)cc2)C2CCCCC2)C(=O)N[C@@H](CCCCN)C(=O)NCCOCCOCCC(N)=O)CC1]

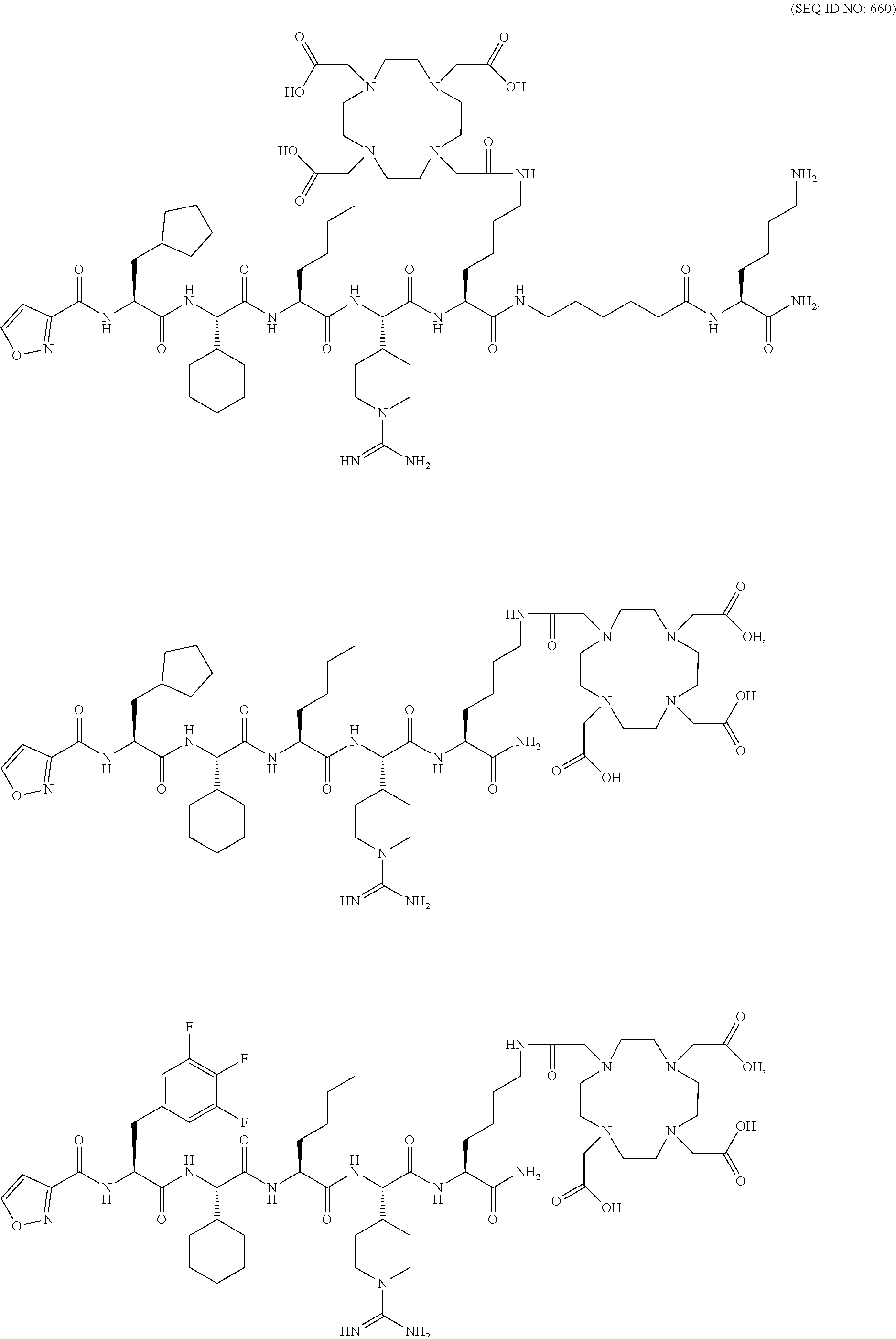

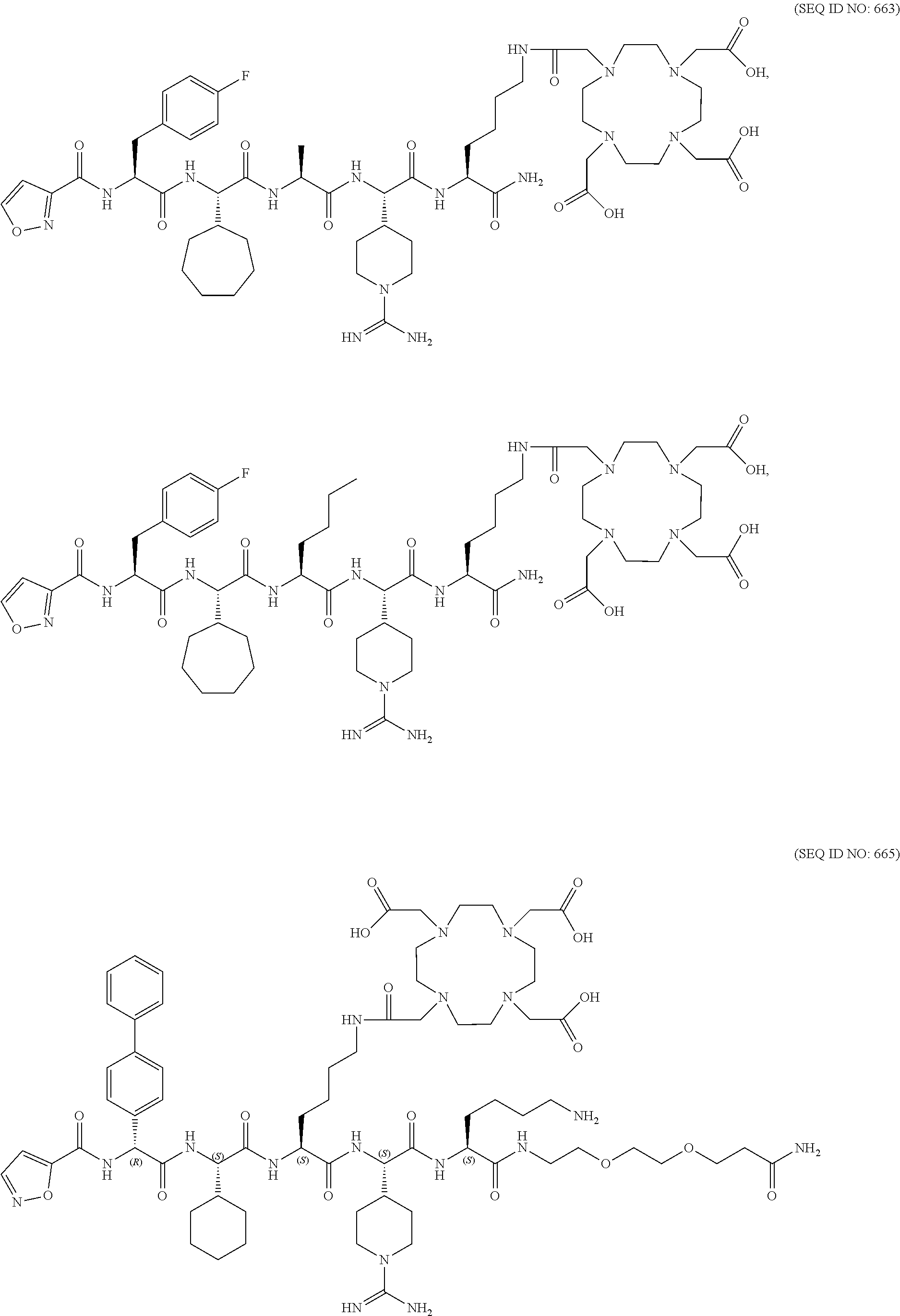

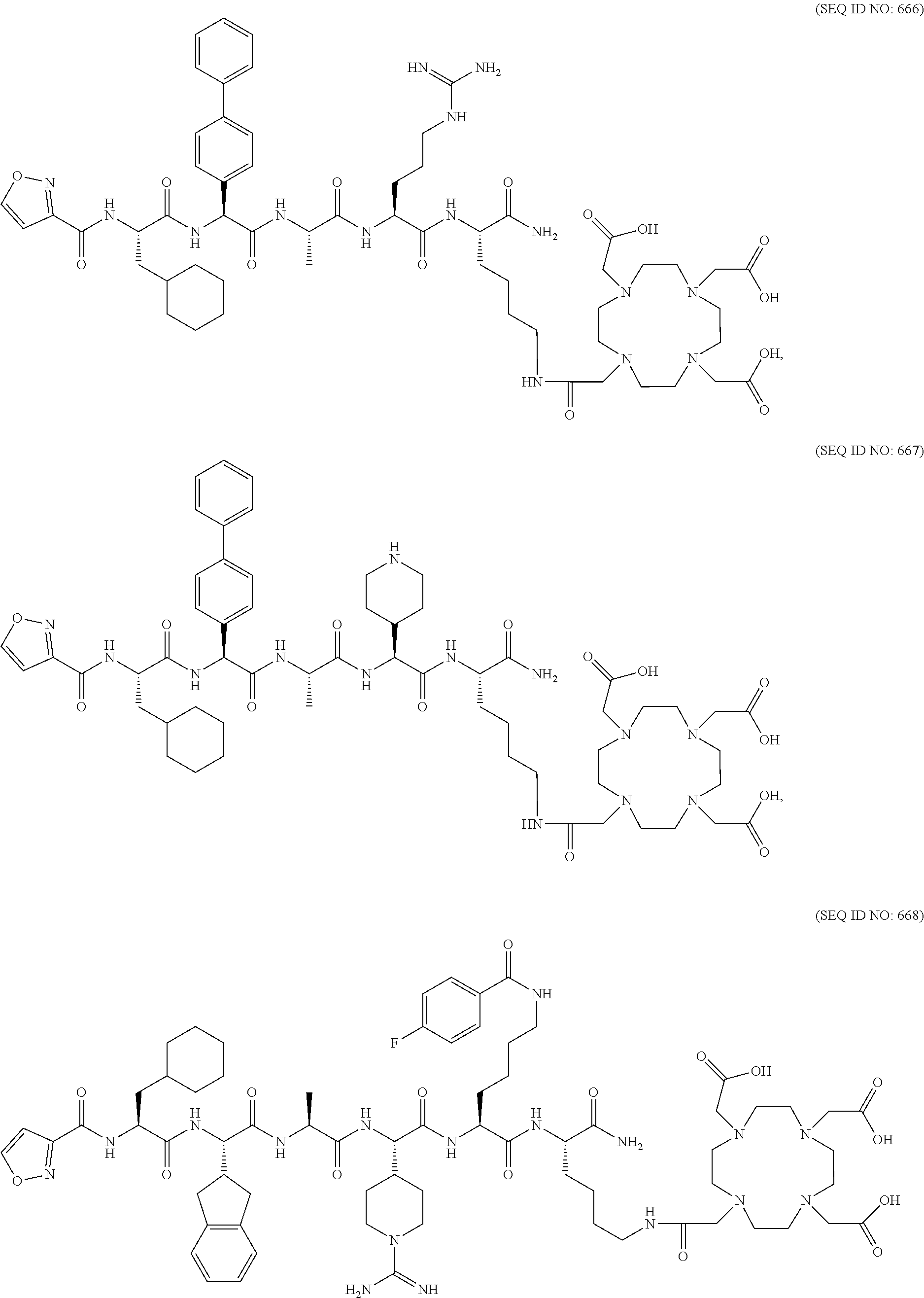

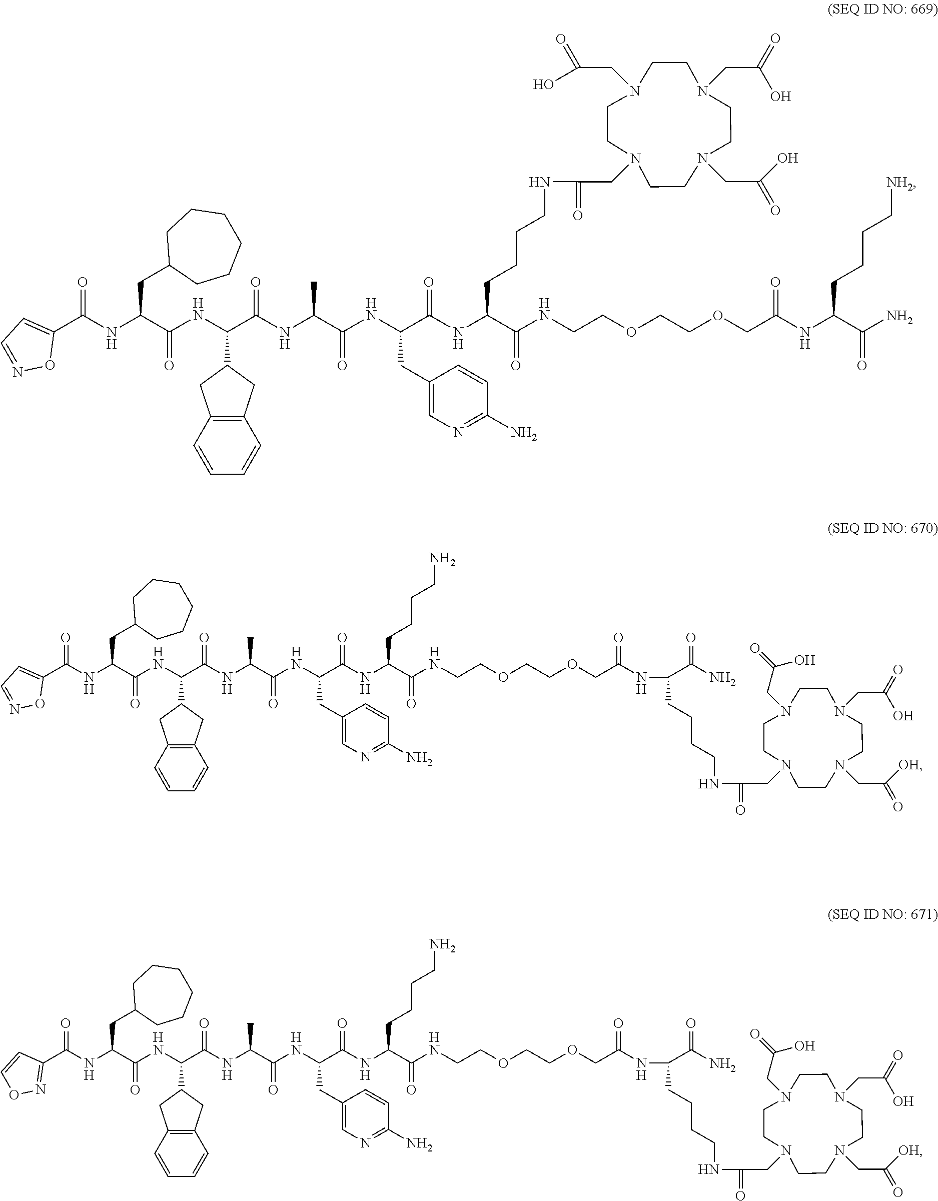

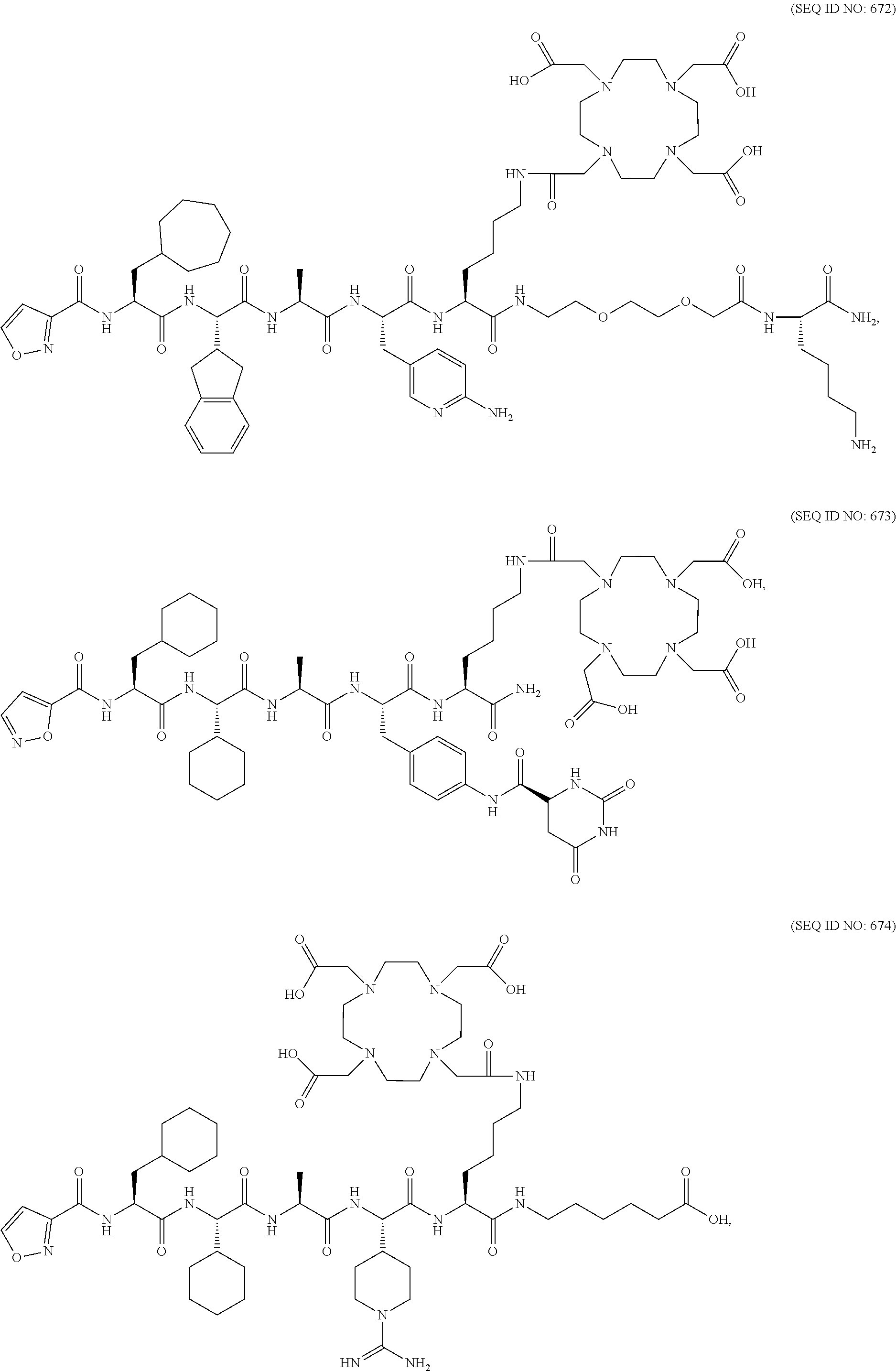

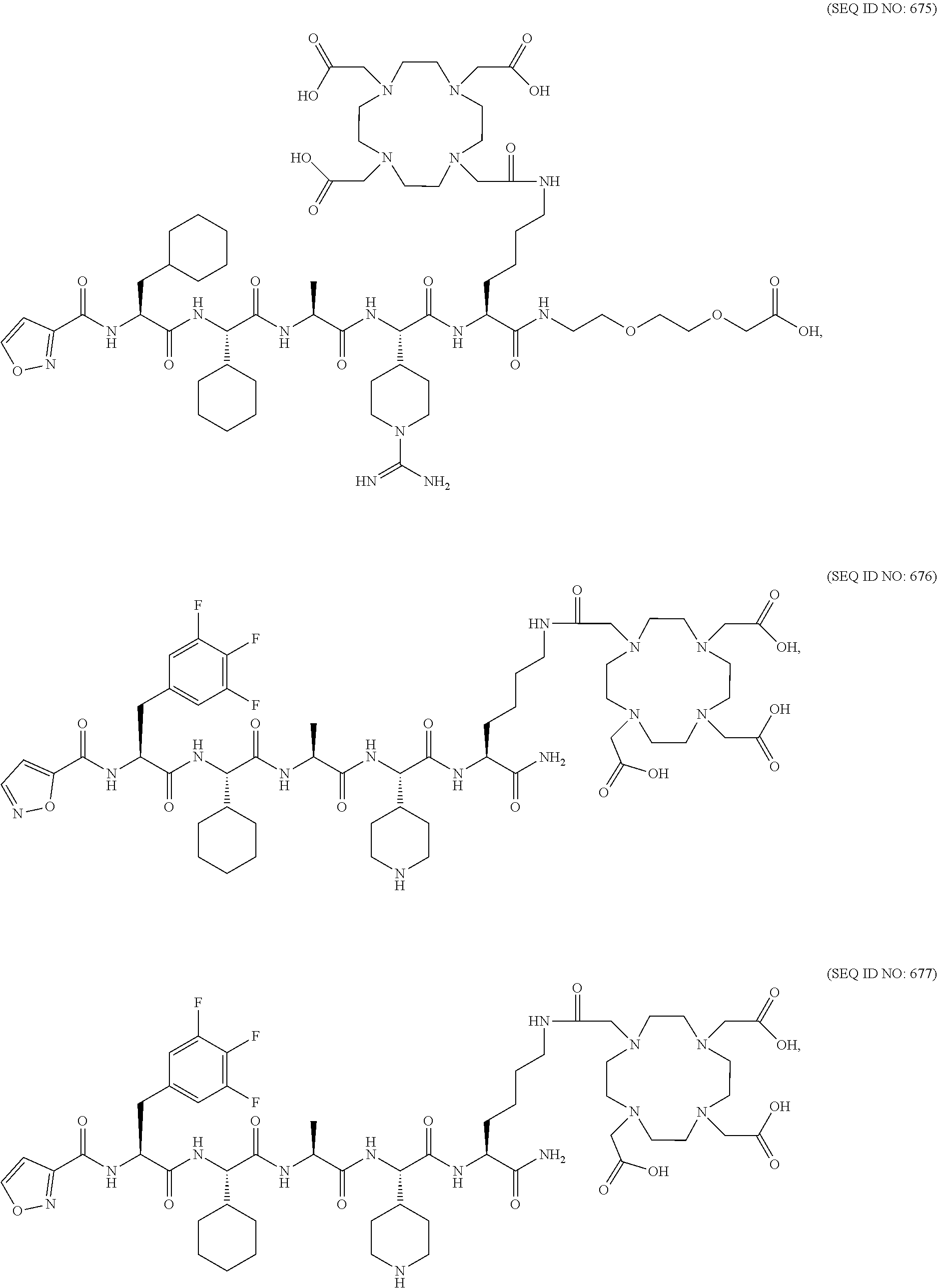

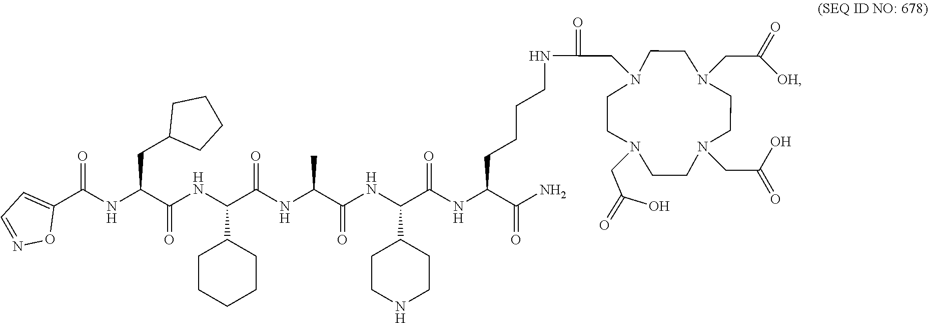 [SMILES: C[C@H](NC(=O)[C@@H](NC(=O)[C@H](CC1CCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

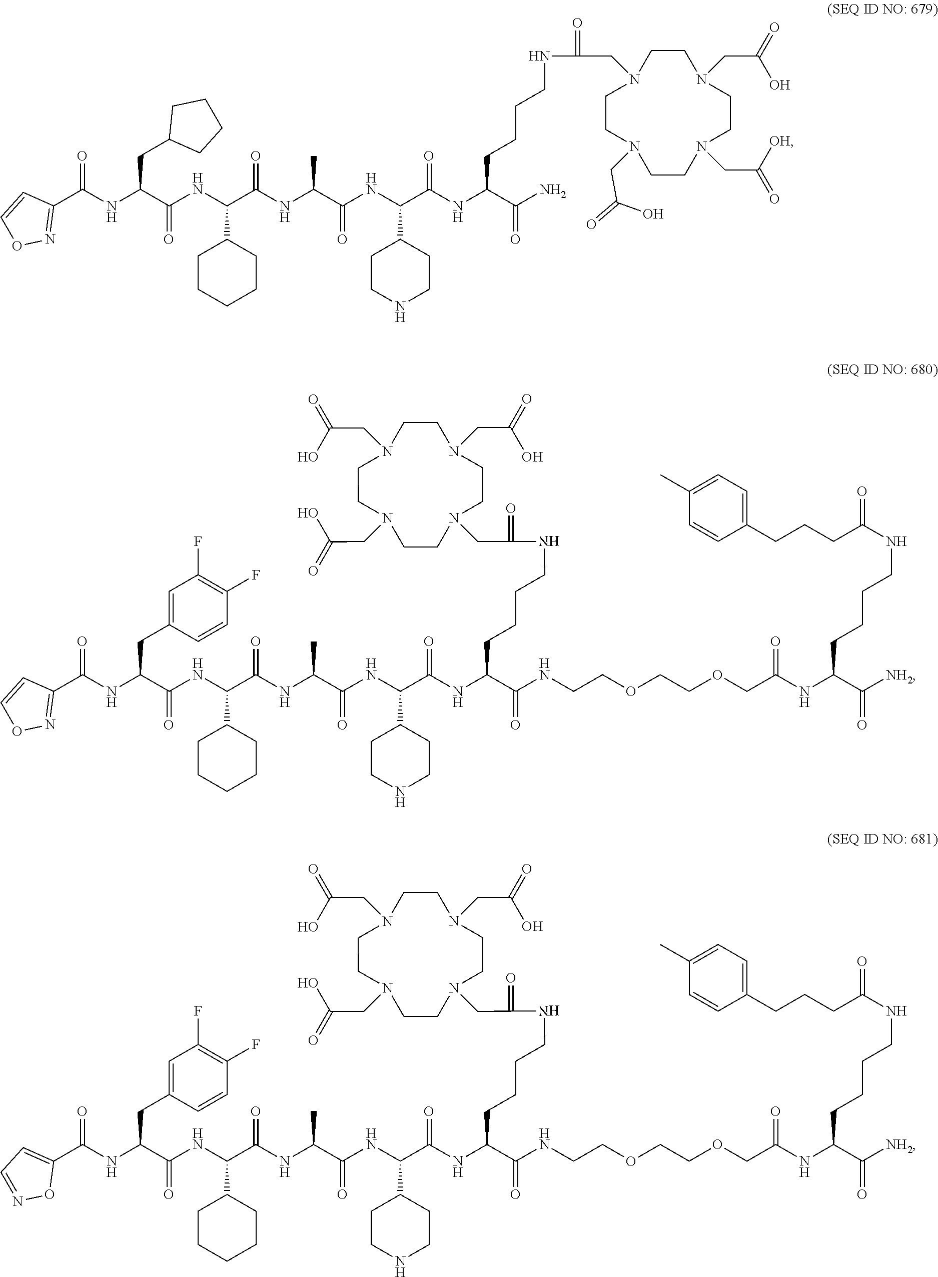

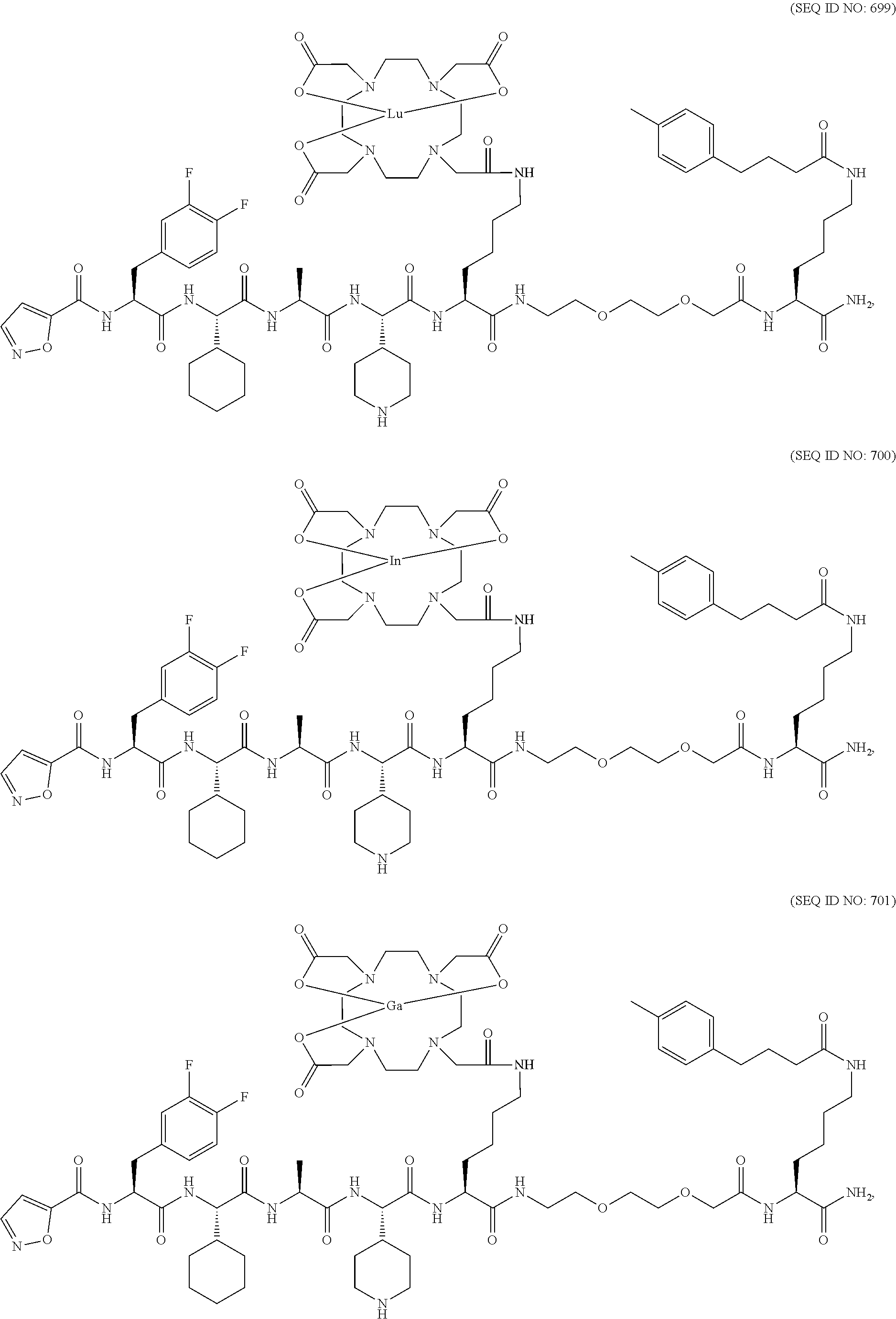

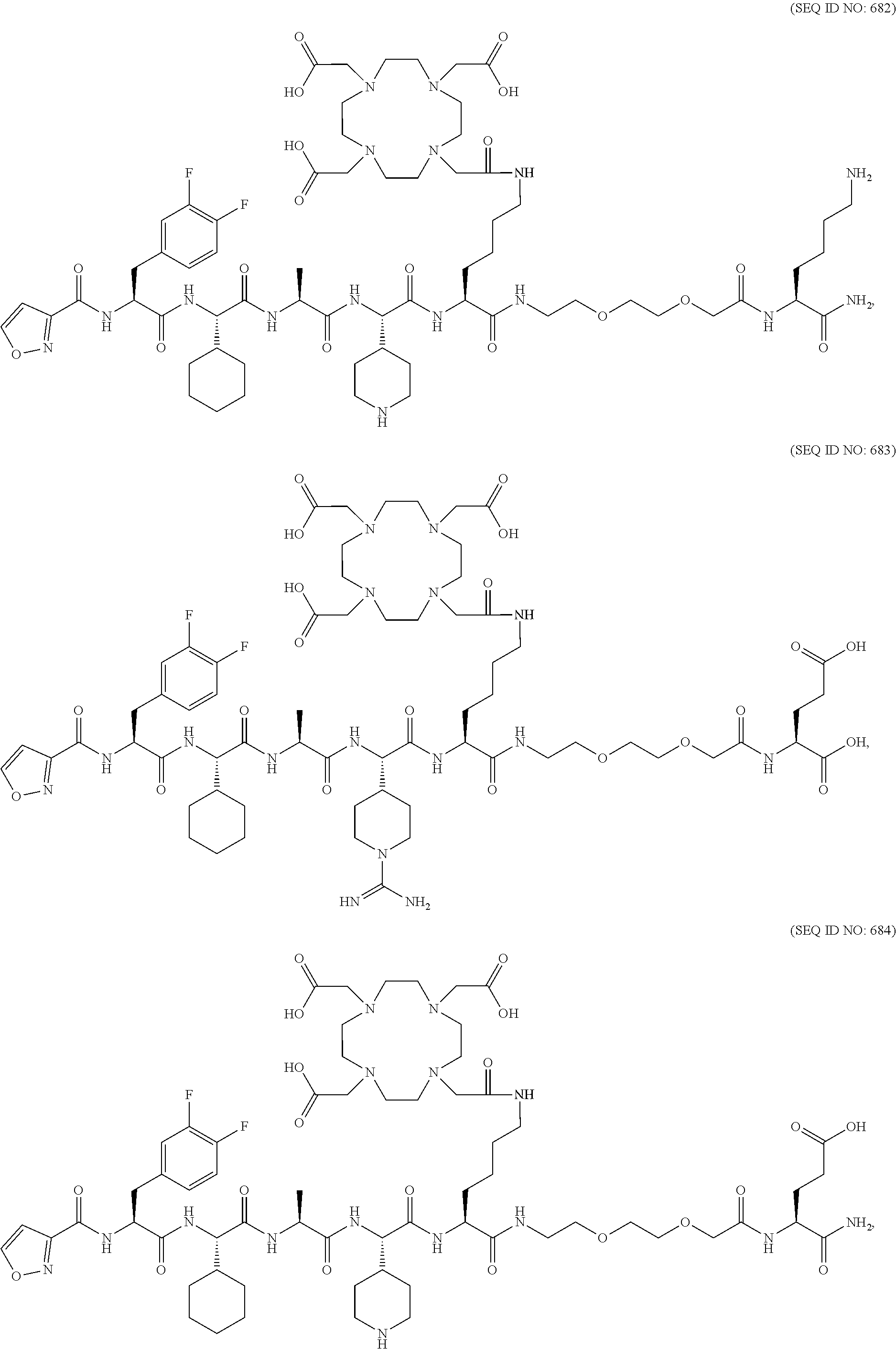

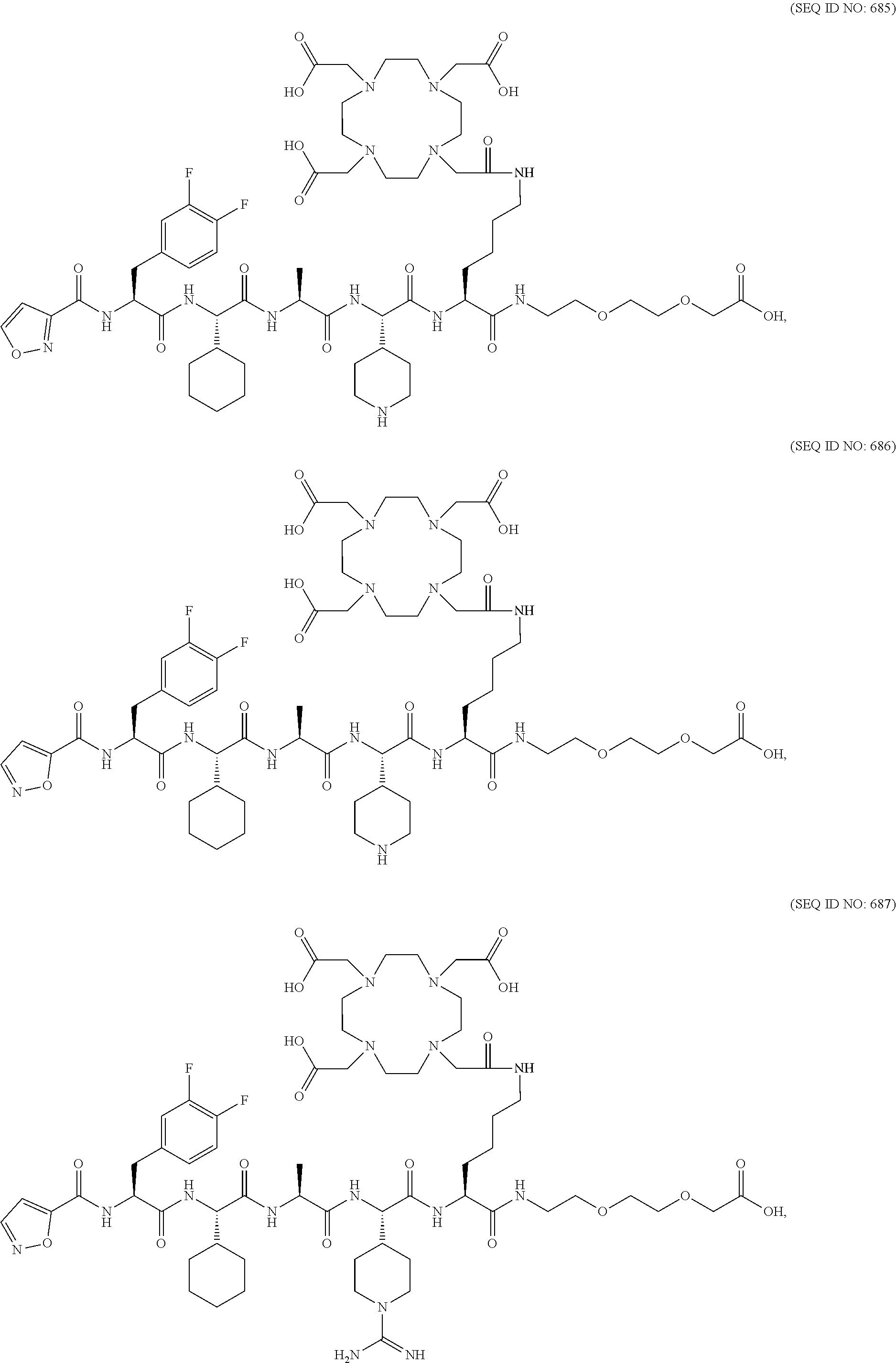

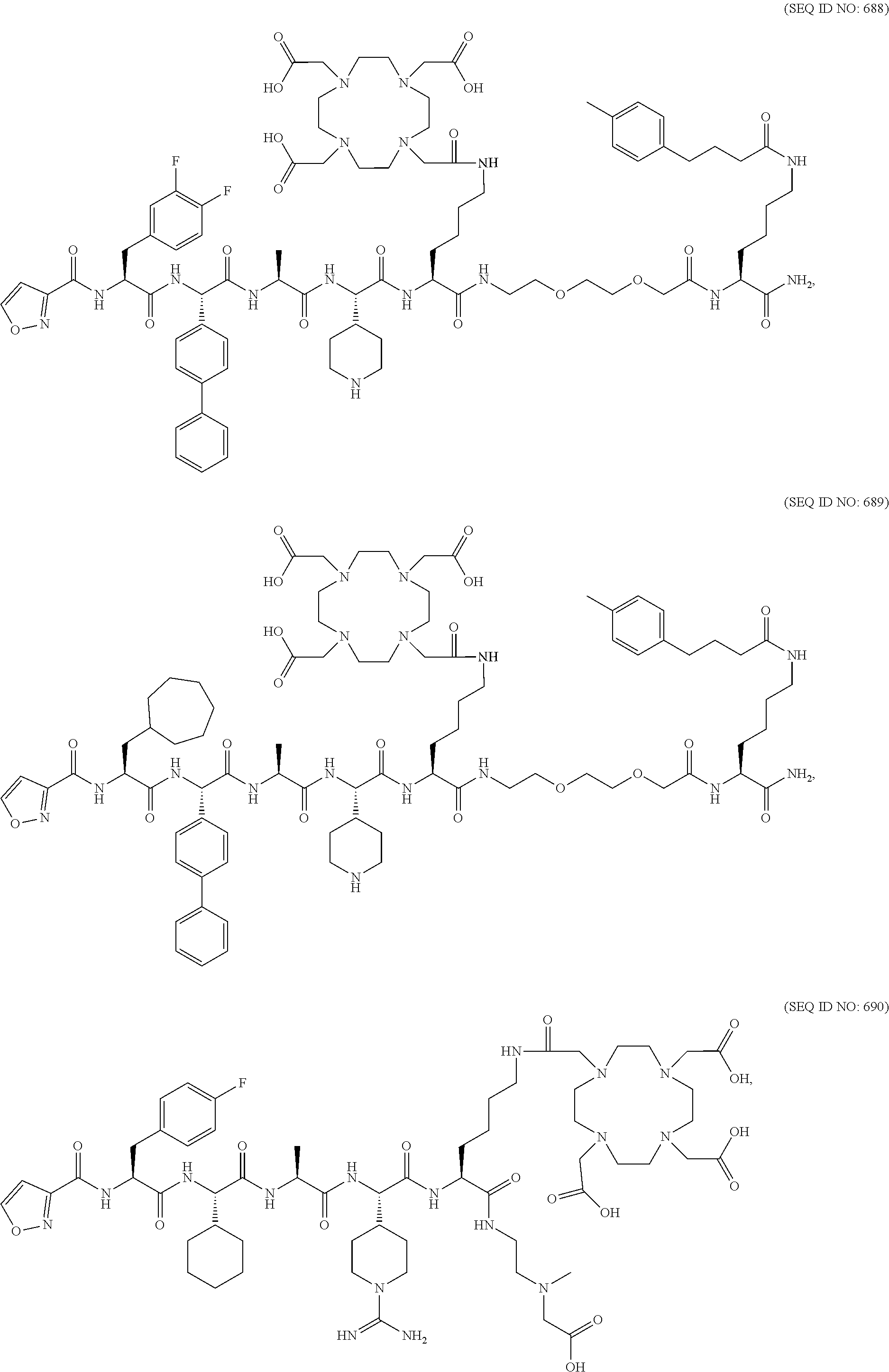

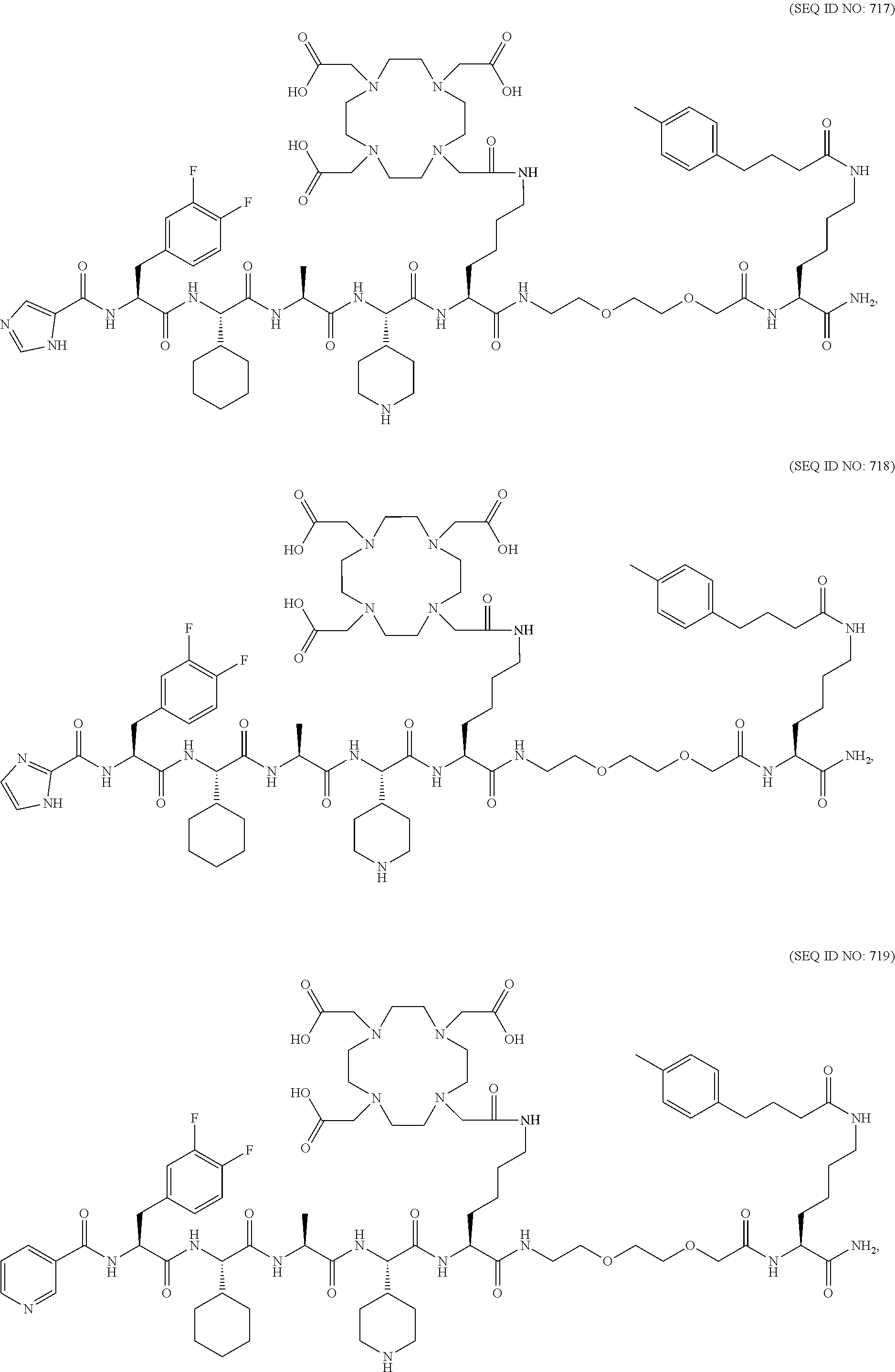

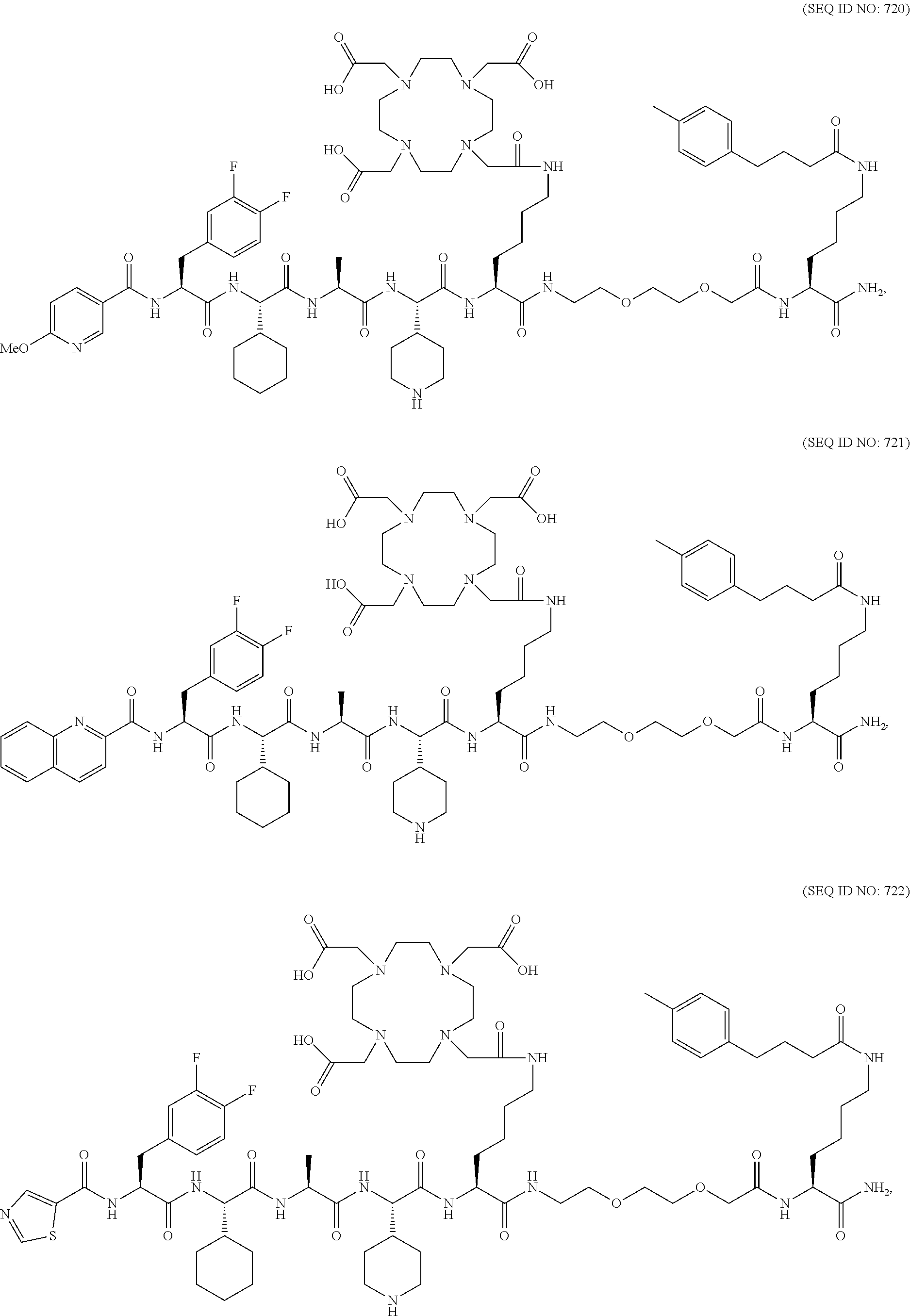

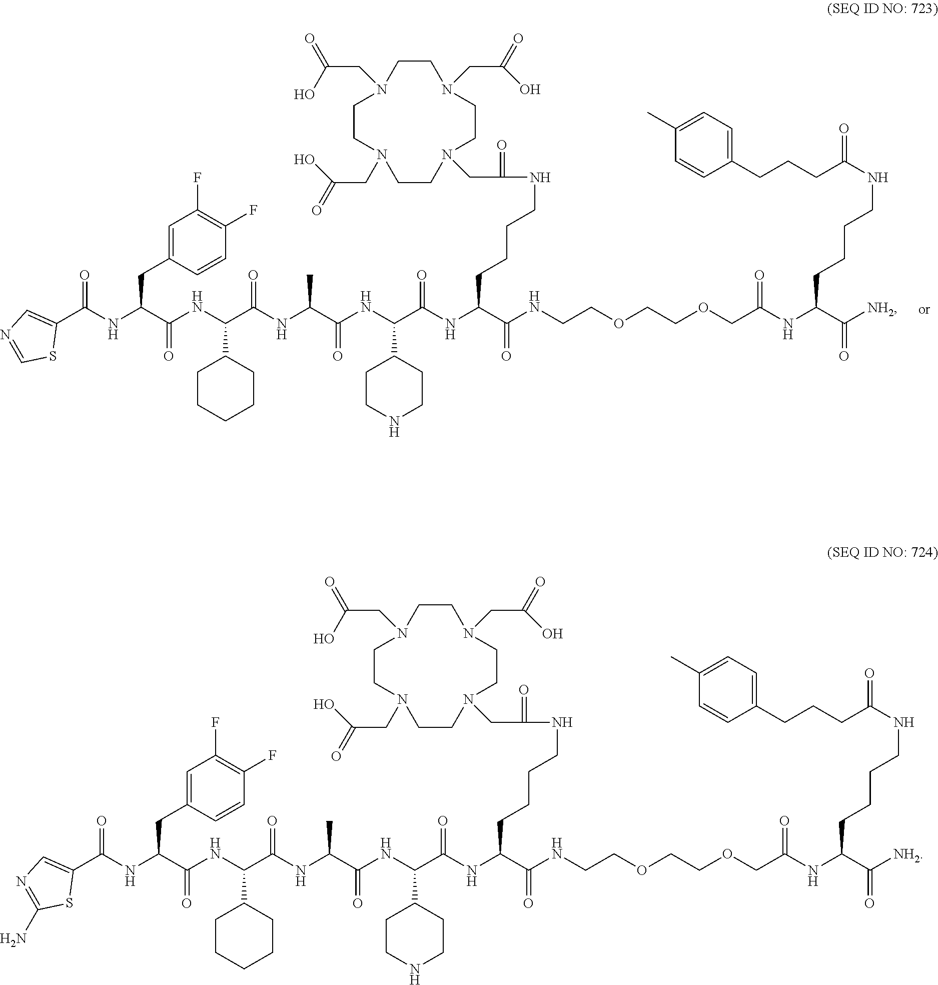 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cncs2)C2CCCCC2)C2CCNCC2)C(N)=O)cc1 ; Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cnc(N)s2)C2CCCCC2)C2CCNCC2)C(N)=O)cc1]

In some embodiments, the compound of Formula (1) or a pharmaceutically acceptable salt thereof, has the following chemical structure, or a pharmaceutically acceptable salt thereof:

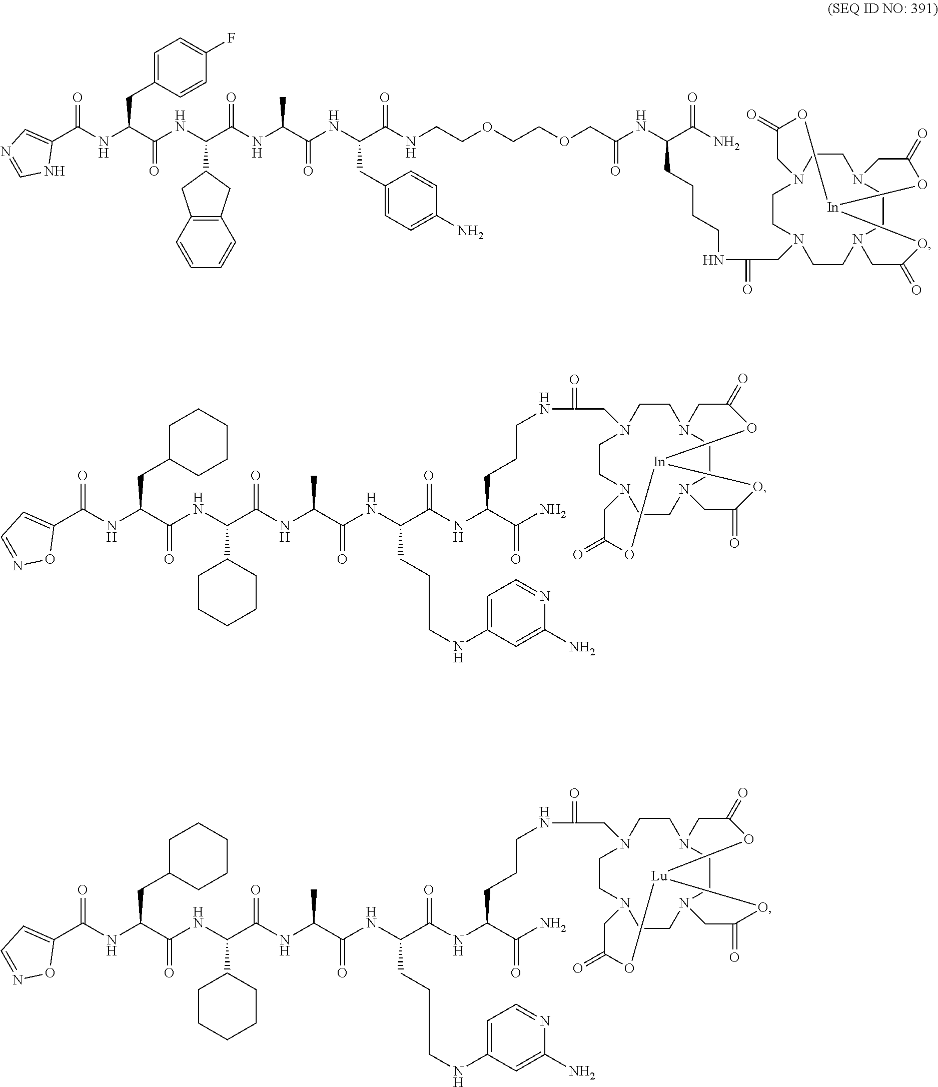

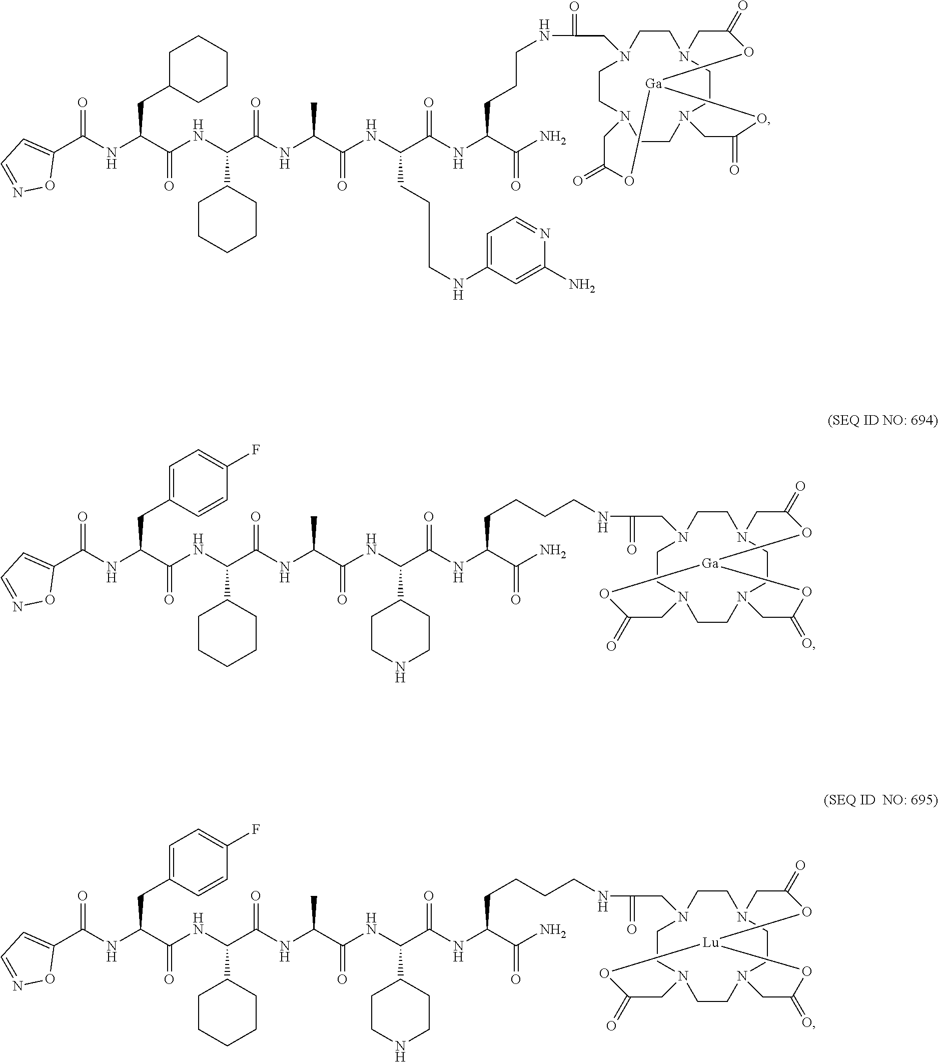

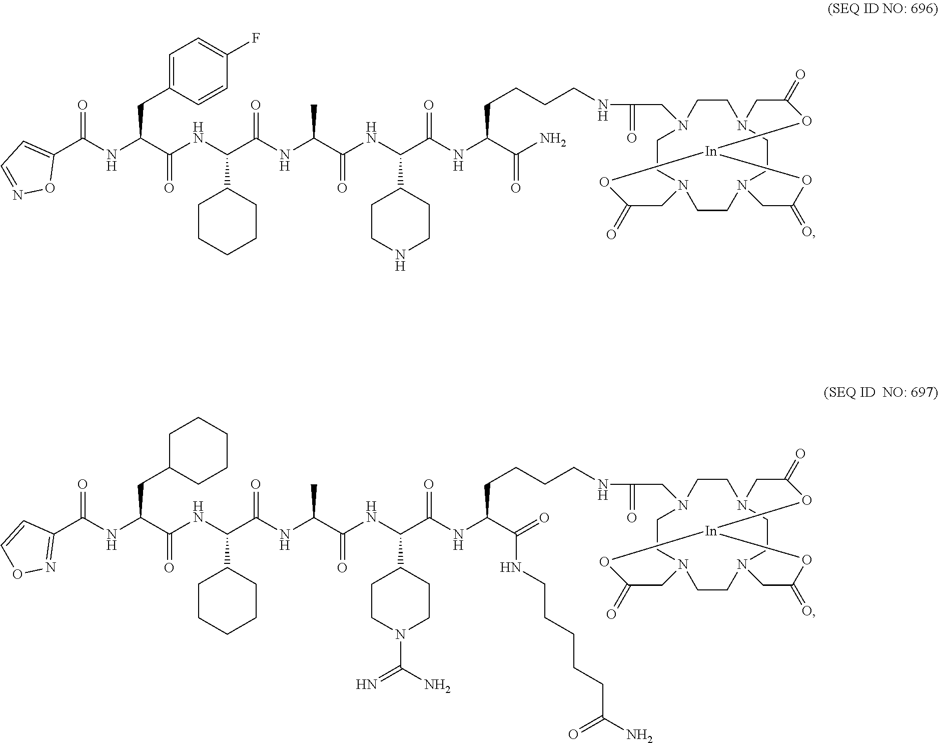

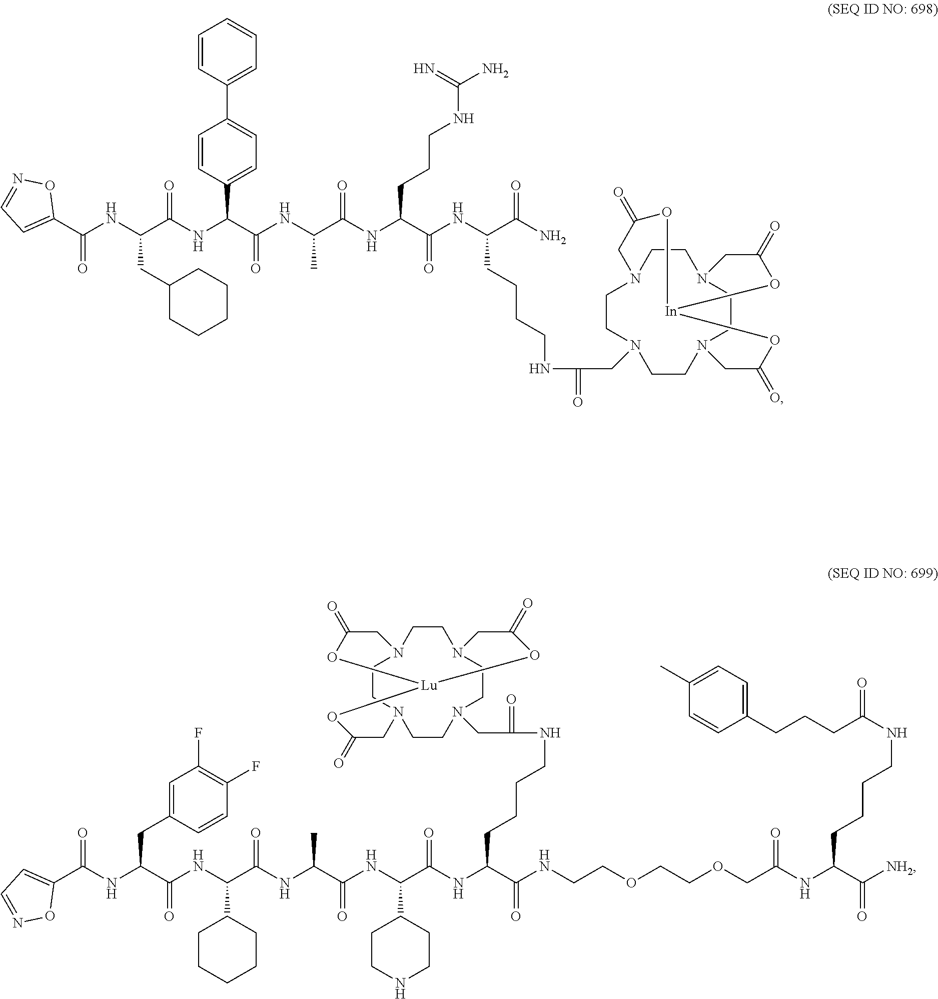

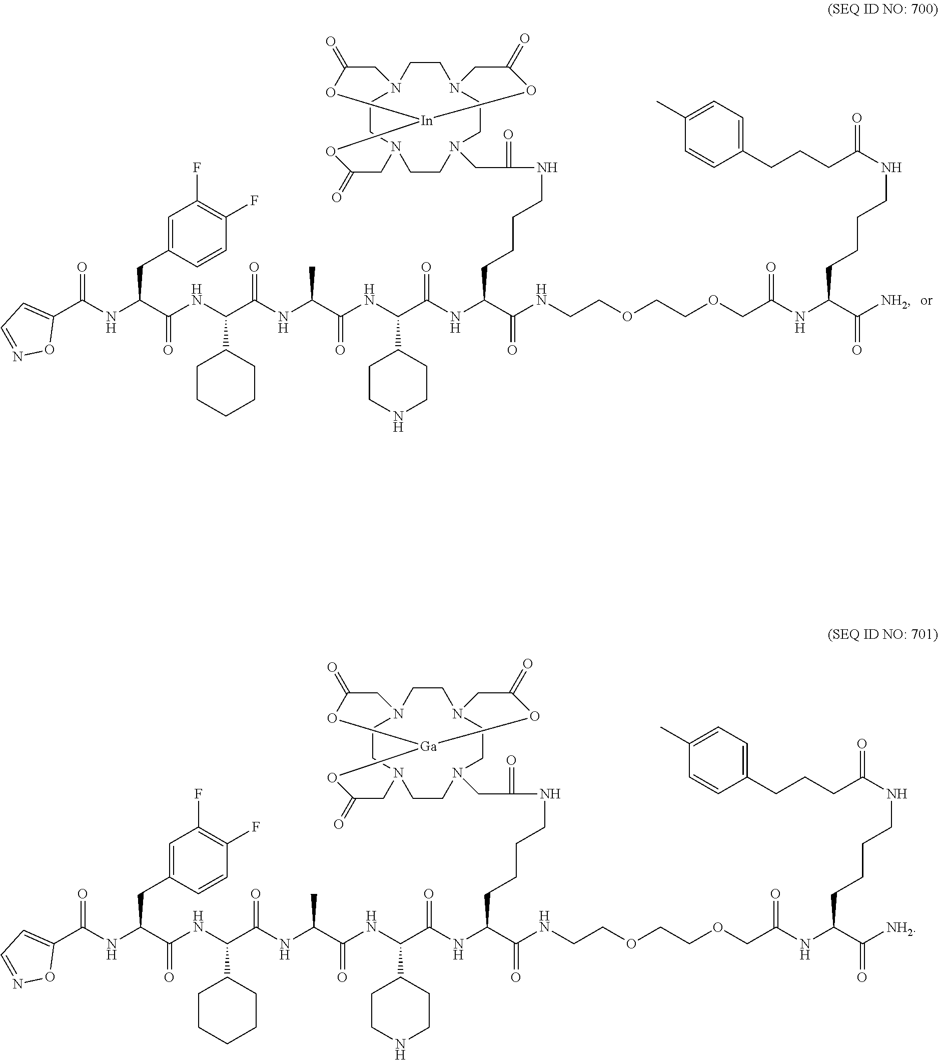

### Representative Protease-Activated Receptor-2 (PAR2) Ligands

In one aspect, described herein is a PAR2 ligand having the structure of Formula (VII), or a pharmaceutically acceptable salt thereof.

In some embodiments, described herein is a compound of Formula (VII), or a pharmaceutically acceptable salt thereof:

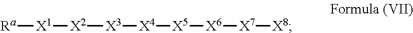

wherein:
R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₅ alkyl, alkoxy, —OH, —NH₂, —NH—C₁-C₆ alkylene-R¹, or —O—C₁-C₆ alkylene-R¹¹, and —R¹¹;
wherein any saturated carbon of R¹³ is optionally substituted with oxo (═O);
R¹¹ is an aryl or heteroaryl optionally substituted with I to 3 substituents that are each independently selected from the group consisting of —F, —Cl, —Br, —C₁-C₃ alkyl, —CF₃, —CN, —OCH₃, —NH₂, and —NO₂;
X¹ is

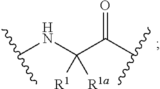

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

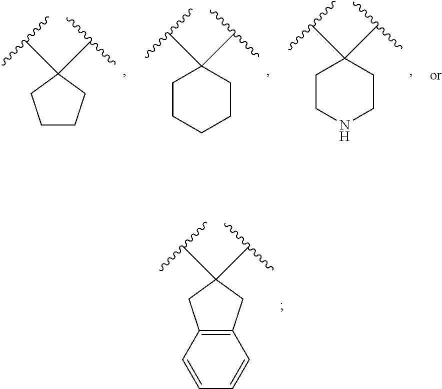 [SMILES: *C1(*)CCCC1 ; *C1(*)CCCCC1 ; *C1(*)CCNCC1 ; *C1(*)Cc2ccccc2C1]

X² is

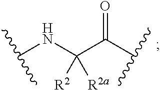

R² is -L²-R^{2b};
L² is absent, —CH₂—, or —CHPh-;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

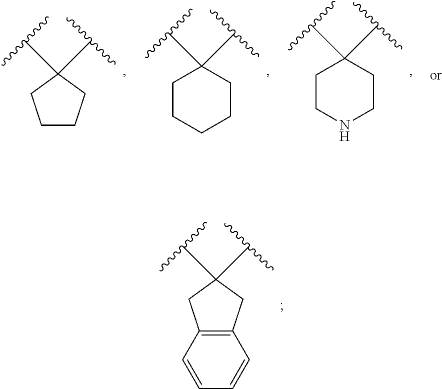 [SMILES: *C1(*)CCCC1 ; *C1(*)CCCCC1 ; *C1(*)CCNCC1 ; *C1(*)Cc2ccccc2C1]

X³ is glycine, alanine (Ala), trifluoromethylalanine, homoalanine, glutamic acid (Glu), glutamine (Gln), ornithine (Orn), lysine (Lys), serine (Ser), N-methylalanine (N-Me-Ala), 2-amino-2-methylpropanoic acid (AIB), 1-aminocyclopropane-1-carboxylic acid (ACC), norleucine (Nle), 3-aminoalanine, 2-amino-6-azidohexanoic acid, 2-amino-6-(4-hexyl-1H-1,2,3-triazol-1-yl)hexanoic acid, 2-amino-6-(4-((2-methoxyethoxy)methyl)-1H-1,2,3-triazol-1-yl)hexanoic acid, 2-amino-6-(4-(cyclopentylmethyl)-1H-1,2,3-triazol-1-yl)hexanoic acid, 2-amino-6-(4-benzyl-1H-1,2,3-triazol-1-yl)hexanoic acid, proline (Pro), cyclopropylglycine, or biphenylalanine (Bip);
X⁴ is arginine (Arg), 3-amino-6-guanidinohexanoic acid, N6-carbamimidoyl-lysine, 2-amino-5-ureidopentanoic acid, homoarginine, N-methyl-arginine, N^{ω}-methyl-arginine, N^{ω}-nitro-arginine, (E)-N^{ω},N^{ω}′-dimethyl-arginine, (E)-N^{ω},N^{ω}′-diethyl-arginine, ornithine (Orn), lysine, phenylalanine (Phe), cyclohexylglycine (Chg), cyclohexylalanine (Cha), norleucine (Nle), 3-amino-alanine (H-Dap-OH), piperidinyl-1-alanine, N⁴,N⁴-dimethyl-asparagine (N⁴,N⁴-dimethyl-Asn), 2-(piperidin-4-yl)-glycine (Gly(piperidin-4-yl)), 3-pyridin-3-yl-beta-alanine (H-βAla(3-pyrid-3-yl)-OH), 2-(1-carbamimidoylpiperidin-4-yl)-glycine (Gly(1-carbamimidoylpiperidin-4-yl)), 3-amino-3-(1-carbamimidoylpiperidin-3-yl)propanoic acid, phenylglycine (Phg), 4-fluorophenylalanine (Phe(4-F)), 4-aminophenylalanine (Phe(4-NH₂, 4-nitrophenylalanine (Phe(4-NO₂)), 4-carbamoylphenylalanine, 4-guanidinophenylalanine, asparagine (Asn), glutamic acid (Glu), proline, 4-ureidophenylalanine, (2-aminoethoxy)phenylalanine (Phe(4-OCH₂CH₂NH₂)), (pyridin-4-yl)alanine (3-Pal), (pyridin-4-yl)alanine (4-Pal), 3-(6-aminopyridin-3-yl)alanine ((3-Pal(6-NH₂)), 2-((2-aminopyridin-4-yl)-λ²-azaneyl)ethyl alanine, N6-nicotinoyl-Lys, 2-oxo-2-(piperazin-1-yl)ethyl alanine, 2-(piperidin-4-yl)ethylalanine, 3-((piperazin-1-yl)propyl)-glycine, 3-((4-methylpiperazin-1-yl)propyl)-glycine, 3-(4-benzylpiperidin-1-yl)propylglycine, 3-((4-benzylpiperazin-1-yl)propyl)-glycine, N6-(1-benzylpiperidine-4-carbonyl)-Lys, 3-((3,4-dihydroisoquinolin-2(1H)-yl)propyl)-glycine, 2-pyridin-4-ylthynyl alanine, (4-(aminomethyl)-1H-1,2λ²,3λ²-triazol-1-yl)methyl)-glycine, (4-(piperazin-1-ylmethyl)-1H-1,2λ²,3λ²-triazol-1-yl)methyl)-glycine, 2-((1H-imidazol-4-yl)ethyl)glycine, or (4-((R)-2,6-dioxohexahydropyrimidine-4-carboxamido)phenyl)alanine;
or X⁴ is 4-methylpiperazine-2-carboxylic acid,
or X⁴ is

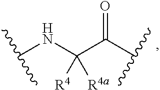

where R⁴ and R^{4a} are taken together with the carbon atom to which they are attached to form

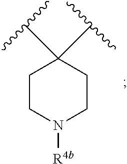

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

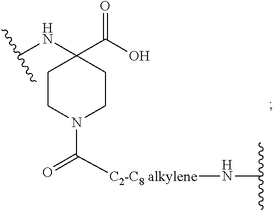

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Ne), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-I)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, 4-amino-1-(methylglycyl)piperidine-4-carboxylic acid, N6-(4-fluorobenzoyl)-Lys, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (The), or

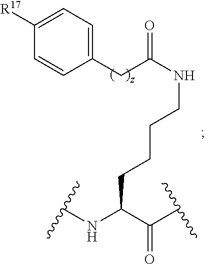

R⁷ is —CH₃ or —I;
z is 1, 2, or 3; and
X⁸ is absent, lysine (Lys), or glutamic acid (Glu);
L⁵, L⁶, and L⁷ are each independently —NR¹⁵-L¹³-L¹⁴-C(═O)— or substituted or unsubstituted piperazinylene-;
   L¹³ is absent, —C₁-C₆ alkylene, substituted or unsubstituted heterocycloalkylene, substituted or unsubstituted cyclohexylene, —(CH₂CH₂O)_{y}—, or —(CH₂CH₂NR¹⁶)_{y}—;
   L¹⁴ is absent, —C₁-C₆ alkylene, substituted or unsubstituted heterocycloalkylene, or substituted or unsubstituted cyclohexylene;
   R¹⁵ and R¹⁶ are each independently selected from H or —C₁-C₄ alkylene;
   y is 1, 2, 3, 4, 5, or 6; and
wherein the compound of Formula (I) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃;
wherein the C-terminal end of the compound of Formula (VII) may be either —COOH or —CONH₂; and
wherein the compound of Formula (VI) is not:

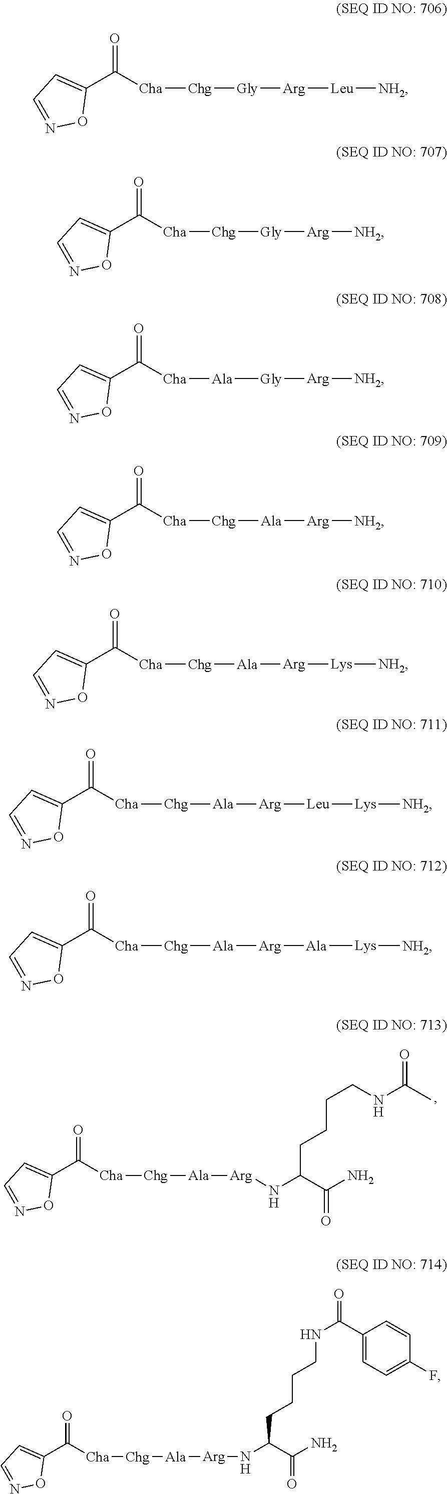

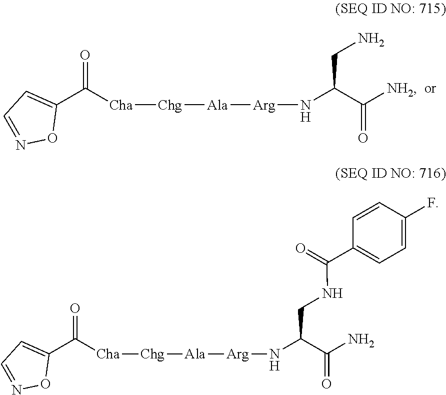

In some embodiments, described herein is a compound of Formula (VII), or a pharmaceutically acceptable salt thereof:

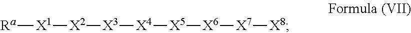

wherein:
R^{a} is —C(═O)—R¹⁰;
R¹⁰ is an aryl, heteroaryl, or partially unsaturated bicyclic heterocyclic ring, each optionally substituted with I to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹, and —R¹;
wherein any saturated carbon of RⁱO is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
X¹ is

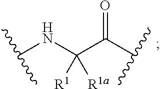

R¹ is -L¹-R^{1b},
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

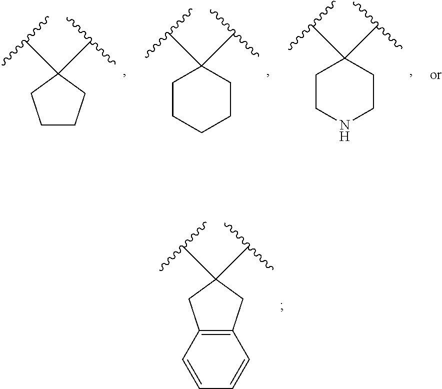 [SMILES: *C1(*)CCCC1 ; *C1(*)CCCCC1 ; *C1(*)CCNCC1 ; *C1(*)Cc2ccccc2C1]

X² is

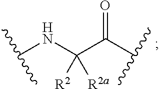

R² is -L²-R^{2b};
L² is absent, —CH₂—, or —CHPh-;
R^{2b} is pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-l-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

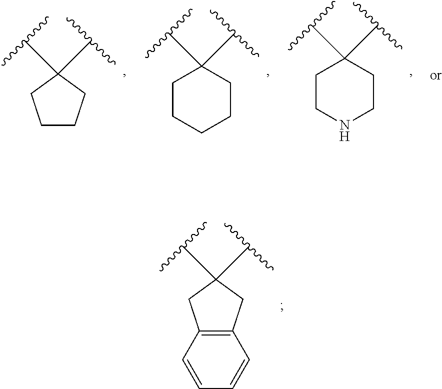 [SMILES: *C1(*)CCCC1 ; *C1(*)CCCCC1 ; *C1(*)CCNCC1 ; *C1(*)Cc2ccccc2C1]

X³ is alanine (Ala), trifluoromethylalanine, homoalanine, glutamic acid (Glu), glutamine (Gln), ornithine (Orn), lysine (Lys), serine (Ser), N-methylalanine (N-Me-Ala), 2-amino-2-methylpropanoic acid (AIB), 1-aminocyclopropane-1-carboxylic acid (ACC), norleucine (Nle), 3-aminoalanine, 2-amino-6-azidohexanoic acid, 2-amino-6-(4-hexyl-1H-1,2,3-triazol-1-yl)hexanoic acid, 2-amino-6-(4-((2-methoxyethoxy)methyl)-1H-1,2,3-triazol-1-yl)hexanoic acid, 2-amino-6-(4-(cyclopentylmethyl)-1H-1,2,3-triazol-1-yl)hexanoic acid, 2-amino-6-(4-benzyl-1H-1,2,3-triazol-l-yl)hexanoic acid, proline (Pro), cyclopropylglycine, or biphenylalanine (Bip);
X⁴ is arginine (Arg), 3-amino-6-guanidinohexanoic acid, N6-carbamimidoyl-lysine, 2-amino-5-ureidopentanoic acid, homoarginine, N-methyl-arginine, No-methyl-arginine, N^{ω}-nitro-arginine, (E)-N^{ω},N^{ω}′-dimethyl-arginine, (E)-N^{ω},N^{ω}′-diethyl-arginine, ornithine (Orn), lysine, phenylalanine (Phe), cyclohexylglycine (Chg), cyclohexylalanine (Cha), norleucine (Nle), 3-amino-alanine (H-Dap-OH), piperidinyl-1-alanine, N⁴,N⁴-dimethyl-asparagine (N⁴,N⁴-dimethyl-Asn), 2-(piperidin-4-yl)-glycine (Gly(piperidin-4-yl)), 3-pyridin-3-yl-beta-alanine (H-βAla(3-pyrid-3-yl)-OH), 2-(1-carbamimidoylpiperidin-4-yl)-glycine (Gly(1l-carbamimidoylpiperidin-4-yl)), 3-amino-3-(1-carbamimidoylpiperidin-3-yl)propanoic acid, phenylglycine (Phg), 4-fluorophenylalanine (Phe(4-F)), 4-aminophenylalanine (Phe(4-NH₂, 4-nitrophenylalanine (Phe(4-NO₂)), 4-carbamoylphenylalanine, 4-guanidinophenylalanine, asparagine (Asn), glutamic acid (Glu), proline, 4-ureidophenylalanine, (2-aminoethoxy)phenylalanine (Phe(4-OCH₂CH₂NH₂)), (pyridin-4-yl)alanine (3-Pal), (pyridin-4-yl)alanine (4-Pal), 3-(6-aminopyridin-3-yl)alanine ((3-Pal(6-NH₂)), 2-((2-aminopyridin-4-yl)-2-azaneyl)ethyl alanine, N6-nicotinoyl-Lys, 2-oxo-2-(piperazin-1-yl)ethyl alanine, 2-(piperidin-4-yl)ethylalanine, 3-((piperazin-1-yl)propyl)-glycine, 3-((4-methylpiperazin-1-yl)propyl)-glycine, 3-(4-benzylpiperidine-1-yl)propylglycine, 3-((4-benzylpiperazin-1-yl)propyl)-glycine, N6-(1-benzylpiperidine-4-carbonyl)-Lys, 3-((3,4-dihydroisoquinolin-2(1H)-yl)propyl)-glycine, 2-pyridin-4-ylthynyl alanine, (4-(aminomethyl)-1H-1,2λ²,3λ²-triazol-1-yl)methyl)-glycine, (4-(piperazin-1-ylmethyl)-1H-1,2λ²,3λ²-triazol-1-yl)methyl)-glycine, 2-((1H-imidazol-4-yl)ethyl)glycine, or (4-((R)-2,6-dioxohexahydropyrimidine-4-carboxamido)phenyl)alanine;
or X⁴ is 4-methylpiperazine-2-carboxylic acid;
or X⁴ is

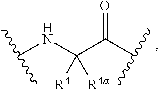

where R⁴ and R^{4a} are taken together with the carbon atom to which they are attached to form

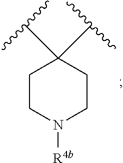

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

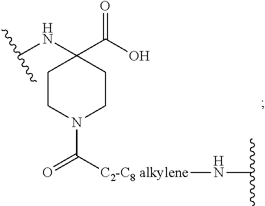

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N3)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-1)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

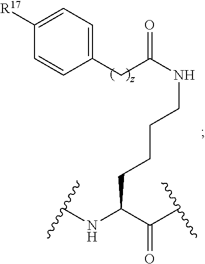

R¹⁷ is —CH₃ or —I;
z is 1, 2, or 3; and
X⁸ is absent, lysine (Lys), or glutamic acid (Glu);
L⁵, L⁶, and L⁷ are each independently —NR¹⁵-L³-L⁴-C(═O)— or substituted or unsubstituted piperazinylene-;
   L¹³ is absent, —C₁-C₆ alkylene, substituted or unsubstituted heterocycloalkylene, or substituted or unsubstituted cyclohexylene, —(CH₂CH₂O)_{y}—, or —(CH₂CH₂NR¹⁶)_{y}—;
   L¹⁴ is absent, —C₁-C₆ alkylene, substituted or unsubstituted heterocycloalkylene, or substituted or unsubstituted cyclohexylene; and
   R¹⁵ and R¹⁶ are each independently selected from H or —C₁-C₄ alkylene;
   y is 1, 2, 3, 4, 5, or 6;
wherein the compound of Formula (VII) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃;
wherein the C-terminal end of the compound of Formula (I) may be either —COOH or —CONH₂; and
wherein when X⁴ is Arginine, X⁵ is not absent, lysine (Lys), leucine (Leu), alanine (Ala), N6-acetyl-lysine, N6-(4-fluorobenzoyl)-Lys, 3-amino-alanine (H-Dap-OH), or (4-fluorobenzamido)methylglycine.

In some embodiments, described herein is a compound of Formula (VII), or a pharmaceutically acceptable salt thereof:

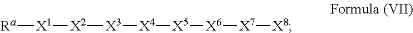

wherein:
R^{a} is —C(═O)—R¹⁰;
R¹¹ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹;
wherein any saturated carbon of R¹⁰ is optionally substituted with oxo (═O);
R¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂:
X¹ is

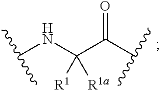

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

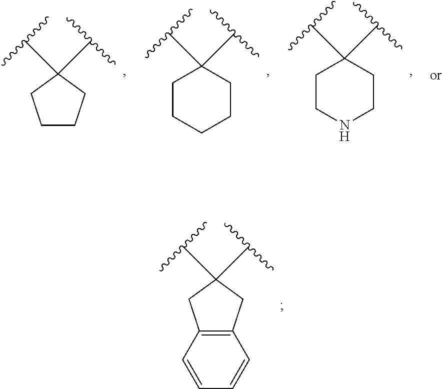 [SMILES: *C1(*)CCCC1 ; *C1(*)CCCCC1 ; *C1(*)CCNCC1 ; *C1(*)Cc2ccccc2C1]

X² is

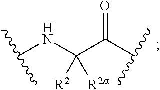

R² is -L²-R^{2b};
L² is absent, —CH₂—, or —CHPh-;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

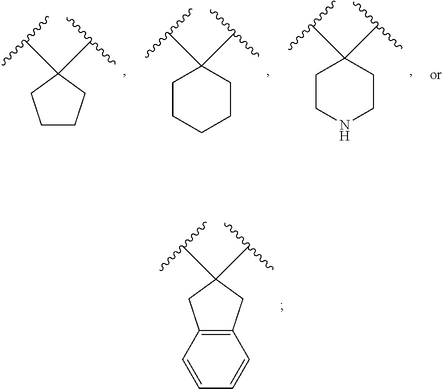 [SMILES: *C1(*)CCCC1 ; *C1(*)CCCCC1 ; *C1(*)CCNCC1 ; *C1(*)Cc2ccccc2C1]

X³ is glycine, alanine (Ala), trifluoromethylalanine, homoalanine, glutamic acid (Glu), glutamine (Gln), ornithine (Orn), lysine (Lys), serine (Ser), N-methylalanine (N-Me-Ala), 2-amino-2-methylpropanoic acid (AID), 1-aminocyclopropane-1-carboxylic acid (ACC), norleucine (Nle), 3-aminoalanine, 2-amino-6-azidohexanoic acid, 2-amino-6-(4-hexyl-1H-1,2,3-triazol-1-yl)hexanoic acid, 2-amino-6-(4-((2-methoxyethoxy)methyl)-1H-1,2,3-triazol-1-yl)hexanoic acid, 2-amino-6-(4-(cyclopentylmethyl)-1H-1,2,3-triazol-1-yl)hexanoic acid, 2-amino-6-(4-benzyl-1H-1,2,3-triazol-1-yl)hexanoic acid, proline (Pro), cyclopropylglycine, or biphenylalanine (Bip);
X⁴ is 3-amino-6-guanidinohexanoic acid, N6-carbamimidoyl-lysine, 2-amino-5-ureidopentanoic acid, homoarginine, N-methyl-arginine, N^{ω}-methyl-arginine, N^{ω}-nitro-arginine, (E)-N^{ω},N^{ω}′-dimethyl-arginine, (E)-N^{ω},N^{ω}′-diethyl-arginine, ornithine (Orn), lysine, phenylalanine (Phe), cyclohexylglycine (Chg), cyclohexylalanine (Cha), norleucine (Ne), 3-amino-alanine (H-Dap-OH), piperidinyl-1-alanine, N⁴,N-dimethyl-asparagine (N⁴,N⁴-dimethyl-Asn), 2-(piperidin-4-yl)-glycine (Gly(piperidin-4-yl)), 3-pyridin-3-yl-beta-alanine (H-βAla(3-pyrid-3-yl)-OH), 2-(1-carbamimidoylpiperidin-4-yl)-glycine (Gly(1-carbamimidoylpiperidin-4-yl)), 3-amino-3-(1-carbamimidoylpiperidin-3-yl)propanoic acid, phenylglycine (Phg), 4-fluorophenylalanine (Phe(4-F)), 4-aminophenylalanine (Phe(4-NH₂)), 4-nitrophenylalanine (Phe(4-NO₂)), 4-carbamoylphenylalanine, 4-guanidinophenylalanine, asparagine (Asn), glutamic acid (Glu), proline, 4-ureidophenylalanine, (2-aminoethoxy)phenylalanine (Phe(4-OCH₂CH₂NH₂)), (pyridin-4-yl)alanine (3-Pal), (pyridin-4-yl)alanine (4-Pal), 3-(6-aminopyridin-3-yl)alanine ((3-Pal(6-NH₂)), 2-((2-aminopyridin-4-yl)-λ²-azaneyl)ethyl alanine, N6-nicotinoyl-Lys, 2-oxo-2-(piperazin-1-yl)ethyl alanine, 2-(piperidin-4-yl)ethylalanine, 3-((piperazin-1-yl)propyl)-glycine, 3-((4-methylpiperazin-1-yl)propyl)-glycine, 3-(4-benzylpiperidin-1-yl)propylglycine, 3-((4-benzylpiperazin-1-yl)propyl)-glycine, N6-(1-benzylpiperidine-4-carbonyl)-Lys, 3-((3,4-dihydroisoquinolin-2(1H)-yl)propyl)-glycine, 2-pyridin-4-ylthynyl alanine, (4-(aminomethyl)-1H-1,2λ²,3λ²-triazol-1-yl)methyl)-glycine, (4-(piperazin-1-ylmethyl)-1H-1,2λ²,3λ²-triazol-1-yl)methyl)-glycine, 2-((1H-imidazol-4-yl)ethyl)glycine, or (4-((R)-2,6-dioxohexahydropyrimidine-4-carboxamido)phenyl)alanine;
or X⁴ is 4-methylpiperazine-2-carboxylic acid;
or X⁴ is

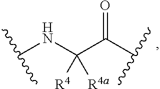

where R⁴ and R^{4a} are taken together with the carbon atom to which they are attached to form

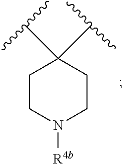

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

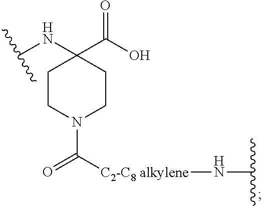

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-I)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

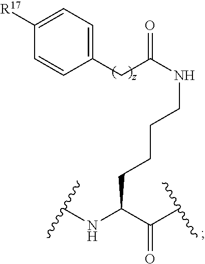

R¹⁷ is —CH₃ or —I;
z is 1, 2, or 3; and
X⁸ is absent, lysine (Lys), or glutamic acid (Glu);
L⁵, L⁶, and L⁷ are each independently —NR¹⁵-L¹³-L¹⁴-C(═O)— or substituted or unsubstituted piperazinylene-;
   L¹³ is absent, —C₁-C₆ alkylene, substituted or unsubstituted heterocycloalkylene, or substituted or unsubstituted cyclohexylene, —(CH₂CH₂O)_{y}—, or —(CH₂CH₂NR¹⁶)_{y}—;
   L¹⁴ is absent, —C₁-C₆ alkylene, substituted or unsubstituted heterocycloalkylene, or substituted or unsubstituted cyclohexylene;
   R¹⁵ and R¹⁶ are each independently selected from H or —C₁-C₄ alkylene; and
y is 1, 2, 3, 4, 5, or 6;
wherein the compound of Formula (I) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (I) may be either —COOH or —CONH₂.

In some embodiments, R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
X¹ is

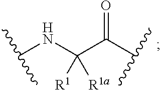

R¹ is -L¹-R^{1b};
L¹ is absent or —CH₂—;
R^{1b} is cyclopentyl, cyclohexyl, cycloheptyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, 2-chlorophenyl, 3-chlorophenyl, 4-chlorophenyl, 2,4-dichlorophenyl, 3,4-dichlorophenyl, or 2,5-dichlorophenyl;
R^{1a} is hydrogen or methyl;
X² is

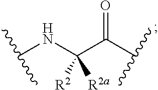

R² is -L²-R^{2b};
L² is absent or —CH₂—;
R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl, or 4′-methyl-[1,1′-biphenyl]-4-yl;
R^{2a} is hydrogen or methyl;
X³ is glycine, (S)-alanine (Ala), or norleucine (Nle);
X⁴ is (S)-arginine (Arg), (S)-ornithine (Orn), (S)-phenylalanine (Phe), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-(pyridin-4-yl)alanine (4-Pal), (S)-4-aminophenylalanine (Phe(4-NH₂)), (S)-3-(6-aminopyridin-3-yl)alanine ((3-Pal(6-NH₂)), or (S)-2-((2-aminopyridin-4-yl)-λ²-azaneyl)ethyl alanine;
X⁵ is alanine (Ala), lysine (Lys), ornithine (Orn),

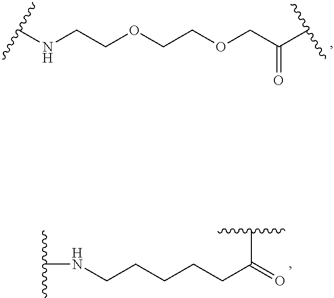 [SMILES: *NCCOCCOCC(*)=O ; *NCCCCCC(*)=O]

N6-(4-fluorobenzoyl)-Lys, β³-homolysine, 3-amino-alanine (H-Dap-OH), 4-nitrophenylalanine (Phe(4-NO₂)), or (4-fluorobenzamido)methylglycine;
X⁶ is absent, lysine (Lys), ornithine (Orn),

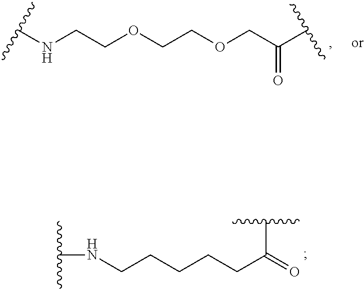 [SMILES: *NCCOCCOCC(*)=O ; *NCCCCCC(*)=O]

X⁷ is absent, lysine (Lys), ornithine (Orn), or

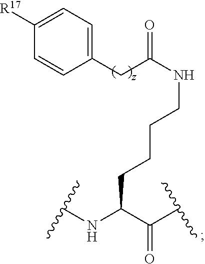

R¹⁷ is —CH₃ or —I;
z is 1, 2, or 3; and
X⁸ is absent;
wherein the compound of Formula (VII) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (VII) may be either —COOH or —CONH₂.

In some embodiments, X¹ is

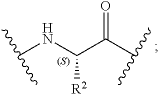

R¹ is -L¹-R^{1b}; L¹ is —CH₂—; and R^{1b} is cyclopentyl, cyclohexyl, cycloheptyl, cyclooctyl, phenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, pyridin-4-yl, 2,3-dihydro-1H-inden-2-yl, 3-cyclopropylphenyl, or [1,1′-biphenyl]-4-yl.

In some embodiments, R^{1b} is cyclohexyl or cycloheptyl. In some embodiments, R^{1b} is phenyl, 4-fluorophenyl, 2,4-difluorophenyl, 3,4-difluorophenyl, 2,5-difluorophenyl, 3,4,5-trifluorophenyl, or perfluorophenyl.

In some embodiments, X² is

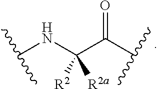

In some embodiments, X² is

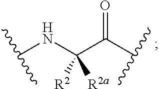

R² is -L²-R^{2b}; L² is absent or —CH₂—; R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl, or 4′-methyl-[1,1′-biphenyl]-4-yl; and R^{2a} is hydrogen or methyl. In some embodiments, R^{2b} is cyclohexyl, cycloheptyl, 2,3-dihydro-1[H-inden-2-yl or [1,1′-biphenyl]-4-yl.

In some embodiments, X² is

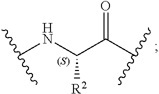

R² is -L²-R^{2b}; L² is absent; and R^{2b} is cyclohexyl or cycloheptyl. In some embodiments, X² is

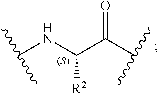

R² is -L²-R^{2b}; L² is —CH₂—; and R^{2b} is 2,3-dihydro-1H-inden-2-yl or [1,1′-biphenyl]-4-yl. In some embodiments, X² is

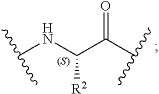

R² is L²-R^{2b}; L² is absent; and R^{2b} is 2,3-dihydro-1H-inden-2-yl or [1,1′-biphenyl]-4-yl.

In some embodiments, X³ is glycine, (S)-alanine (Ala), (S)-trifluoromethylalanine, (S)-homoalanine, (S)-glutamic acid (Glu), (S)-glutamine (Gln), (S)-ornithin (Orn), (S)-lysine (Lys), (S)-serine (Ser), (S)—N-methylalanine (N-Me-Ala), 2-amino-2-methylpropanoic acid (AID), 1-aminocyclopropane-1-carboxylic acid (ACC), (S)-norleucine (Nle), (S)-3-aminoalanine, (S)-2-amino-6-azidohexanoic acid, (S)-2-amino-6-(4-hexyl-1H-1,2,3-triazol-1-yl)hexanoic acid, (S)-2-amino-6-(4-((2-methoxyethoxy)methyl)-1H-1,2,3-triazol-1-yl)hexanoic acid, (S)-2-amino-6-(4-(cyclopentylmethyl)-1H-1,2,3-triazol-1-yl)hexanoic acid, (S)-2-amino-6-(4-benzyl-1H-1,2,3-triazol-1-yl)hexanoic acid, (S)-proline (Pro), (S)-cyclopropylglycine, or (S)-biphenylalanine (Bip). In some embodiments, X³ is glycine, (S)-alanine (Ala), or norleucine (Nle). In some embodiments, X³ is (S)-alanine (Ala) or (S)-norleucine (Nle). In some embodiments, X³ is (S)-alanine (Ala). In some embodiments, X³ is (S)-norleucine (Nle). In some embodiments, X³ is alanine (Ala). In some embodiments, X³ is (S)-alanine (Ala). In some embodiments, X³ is (S)-trifluoromethylalanine. In some embodiments, X³ is (S)-homoalanine. In some embodiments, X³ is (S)-Orn. In some embodiments, X³ is (S)-lysine (Lys). In some embodiments, X³ is (S)-serine (Ser). In some embodiments, X³ is (S)—N-methylalanine (N-Me-Ala). In some embodiments, X³ is 2-amino-2-methylpropanoic acid (AIB). In some embodiments, X³ is 1-aminocyclopropane-1-carboxylic acid (ACC). In some embodiments, X³ is norleucine (Nle). In some embodiments, X³ is (S)-3-aminoalanine. In some embodiments, X³ is (S)-proline (Pro). In some embodiments, X³ is (S)-cyclopropylglycine. In some embodiments, X³ is (S)-biphenylalanine (Bip).

In some embodiments, X³ is (S)-alanine (Ala) or (S)-norleucine (Nle). In some embodiments, X³ is (S)-alanine (Ala).

In some embodiments, X⁴ is (S)-arginine (Arg), (S)-3-amino-6-guanidinohexanoic acid, N6-carbamimidoyl-(S)-lysine, (S)-2-amino-5-ureidopentanoic acid, (S)-homoarginine, N-methyl-(S)-arginine, N^{ω}-methyl-(S)-arginine, N^{ω}-nitro-(S)-arginine, (E)-N^{ω},N^{ω}′-dimethyl-(S)-arginine, (E)-N^{ω},N^{ω}′-diethyl-(S)-arginine, (S)-ornithine (Orn), (S)-lysine, (S)-phenylalanine (Phe), (S)-cyclohexylglycine (Chg), (S)-cyclohexylalanine (Cha), norleucine (Nle), (S)-3-amino-alanine (H-Dap-OH), piperidinyl-1-(S)-alanine, N⁴,N⁴-dimethyl-(S)-asparagine (N⁴,N⁴-dimethyl-Asn), (S)-2-(piperidin-4-yl)-glycine (Gly(piperidin-4-yl)), (S)-3-pyridin-3-yl-beta-alanine (I-βAla(3-pyrid-3-yl)-OH), (S)-2-(1-carbamimidoylpiperidin-4-yl)-glycine (Gly(1-carbamimidoylpiperidin-4-yl)), (S)-3-amino-3-(1-carbamimidoylpiperidin-3-yl)propanoic acid, (S)-phenylglycine (Phg), (S)-4-fluorophenylalanine (Phe(4-F)), (S)-4-aminophenylalanine (Phe(4-NH₂)), (S)-4-nitrophenylalanine (Phe(4-NO₂)), (S)-4-carbamoylphenylalanine, (S)-4-guanidinophenylalanine, (S)-asparagine (Asn), (S)-glutamic acid (Glu), (S)-proline, (S)-4-ureidophenylalanine, (S)-(2-aminoethoxy)phenylalanine (Phe(4-OCH₂CH₂NH₂)), (S)-(pyridin-4-yl)alanine (3-Pal), (S)-(pyridin-4-yl)alanine (4-Pal), (S)-3-(6-aminopyridin-3-yl)alanine ((3-Pal(6-NH₂)), (S)-2-((2-aminopyridin-4-yl)-2-azaneyl)ethyl alanine, N6-nicotinoyl-Lys, 2-oxo-2-(piperazin-1-yl)ethyl alanine, (S)-2-(piperidin-4-yl)ethylalanine, (S)-3-((piperazin-1-yl)propyl)-glycine, (S)-3-((4-methylpiperazin-1-yl)propyl)-glycine, (S)-3-(4-benzylpiperidin-1-yl)propylglycine, (S)-3-((4-benzylpiperazin-1-yl)propyl)-glycine, N6-(1-benzylpiperidine-4-carbonyl)-Lys, (S)-3-((3,4-dihydroisoquinolin-2(1H)-yl)propyl)-glycine, (S)-2-pyridin-4-ylthynyl alanine, (S)-(4-(aminomethyl)-1H-1,2λ²,3λ²-triazol-1-yl)methyl)-glycine, (S)-(4-(piperazin-1-ylmethyl)-1H-1,2λ²,3λ²-triazol-1-yl)methyl)-glycine, (S)-2-((1H-imidazol-4-yl)ethyl)glycine, or(S)-(4-((R)-2,6-dioxohexahydropyrimidine-4-carboxamido)phenyl)alanine;
or X⁴ is (S)-4-methylpiperazine-2-carboxylic acid;
or X⁴ is

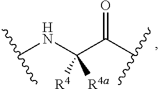

where R⁴ and R^{4a} are taken together with the carbon atom to which they are attached to form

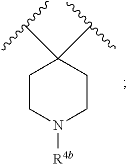

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

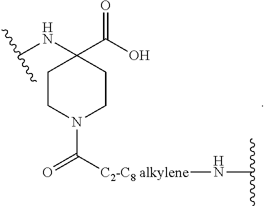

In some embodiments, X⁴ is (S)-3-amino-6-guanidinohexanoic acid, N6-carbamimidoyl-(S)-lysine, (S)-2-amino-5-ureidopentanoic acid, (S)-homoarginine, N-methyl-(S)-arginine, N^{ω}-methyl-(S)-arginine, N^{ω}-nitro-(S)-arginine, (E)-N^{ω},N^{ω}′-dimethyl-(S)-arginine, (E)-N^{ω},N ^{ω}′-diethyl-(S)-arginine, (S)-ornithine (Orn), (S)-lysine, (S)-phenylalanine (Phe), (S)-cyclohexylglycine (Chg), (S)-cyclohexylalanine (Cha), norleucine (Nle), (S)-3-amino-alanine (H-Dap-OH), piperidinyl-1-(S)-alanine, N⁴,N⁴-dimethyl-(S)-asparagine (N⁴,N⁴-dimethyl-Asn), (S)-2-(piperidin-4-yl)-glycine (Gly(piperidin-4-yl)), (S)-3-pyridin-3-yl-beta-alanine (H-βAla(3-pyrid-3-yl)-OH), (S)-2-(I-carbamimidoylpiperidin-4-yl)-glycine (Gly(1-carbamimidoylpiperidin-4-yl)), (S)-3-amino-3-(1-carbamimidoylpiperidin-3-yl)propanoic acid, (S)-phenylglycine (Phg), (S)-4-fluorophenylalanine (Phe(4-F)), (S)-4-aminophenylalanine (Phe(4-NH₂)), (S)-4-nitrophenylalanine (Phe(4-NO₂)), (S)-4-carbamoylphenylalanine, (S)-4-guanidinophenylalanine, (S)-asparagine (Asn), (S)-glutamic acid (Glu), (S)-proline, (S)-4-ureidophenylalanine, (S)-(2-aminoethoxy)phenylalanine (Phe(4-OCH₂CH₂NH₂)), (S)-(pyridin-4-yl)alanine (3-Pal), (S)-(pyridin-4-yl)alanine (4-Pal), (S)-3-(6-aminopyridin-3-yl)alanine ((3-Pal(6-NH₂)), (S)-2-((2-aminopyridin-4-yl)-λ²-azaneyl)ethyl alanine, N6-nicotinoyl-Lys, 2-oxo-2-(piperazin-1-yl)ethyl alanine, (S)-2-(piperidin-4-yl)ethylalanine, (S)-3-((piperazin-1-yl)propyl)-glycine, (S)-3-((4-methylpiperazin-1-yl)propyl)-glycine, (S)-3-(4-benzylpiperidin-1-yl)propylglycine, (S)-3-((4-benzylpiperazin-1-yl)propyl)-glycine, N6-(1-benzylpiperidine-4-carbonyl)-Lys, (S)-3-((3,4-dihydroisoquinolin-2(1H)-yl)propyl)-glycine, (S)-2-pyridin-4-ylthynyl alanine, (S)-(4-(aminomethyl)-1H-1,2λ²,3λ²-triazol-1-yl)methyl)-glycine, (S)-(4-(piperazin-1-ylmethyl)-H-1,2λ²,3λ²-triazol-1-yl)methyl)-glycine, (S)-2-((1H-imidazol-4-yl)ethyl)glycine, or (S)-(4-((R)-2,6-dioxohexahydropyrimidine-4-carboxamido)phenyl)alanine;
or X⁴ is (S)-4-methylpiperazine-2-carboxylic acid;
or X⁴ is

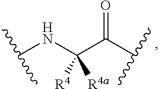

where R⁴ and R^{4a} are taken together with the carbon atom to which they are attached to form

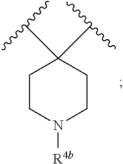

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

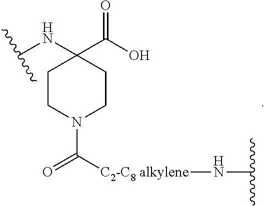

In some embodiments, X⁴ is (S)-arginine (Arg), (S)-ornithine (Orn), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-3-(6-aminopyridin-3-yl)alanine ((3-Pal(6-N112)), or (S)-2-((2-aminopyridin-4-yl)-λ²-azaneyl)ethyl alanine. In some embodiments, X⁴ is (S)-arginine (Arg). In some embodiments, X⁴ is (S)-2-(1-carbamimidoylpiperidin-4-yl)-glycine (Gly(1-carbamimidoylpiperidin-4-yl)). In some embodiments, X⁴ is (S)-2-amino-3-(4-aminophenyl)propanoic acid (Phe(4-NH₂)), (S)-2-amino-3-(6-aminopyridin-3-yl)propanoic acid, or (S)-2-((2-aminopyridin-4-yl)-X²-azaneyl)ethyl alanine.

In some embodiments, X⁴

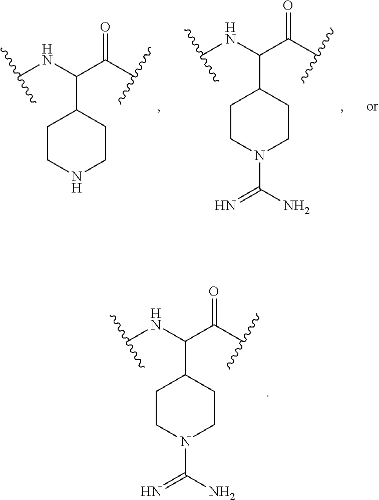 [SMILES: *NC(C(*)=O)C1CCNCC1 ; *NC(C(*)=O)C1CCN(C(=N)N)CC1 ; *NC(C(*)=O)C1CCN(C(=N)N)CC1]

In some embodiments, X⁴ is

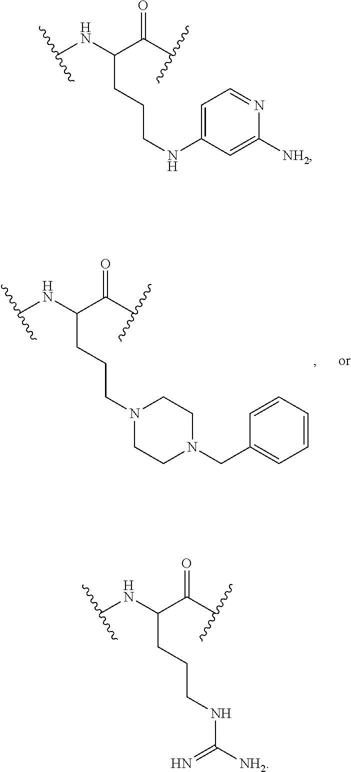 [SMILES: *NC(CCCNc1ccnc(N)c1)C(*)=O ; *NC(CCCN1CCN(Cc2ccccc2)CC1)C(*)=O ; *NC(CCCNC(=N)N)C(*)=O]

In some embodiments, X⁴ is

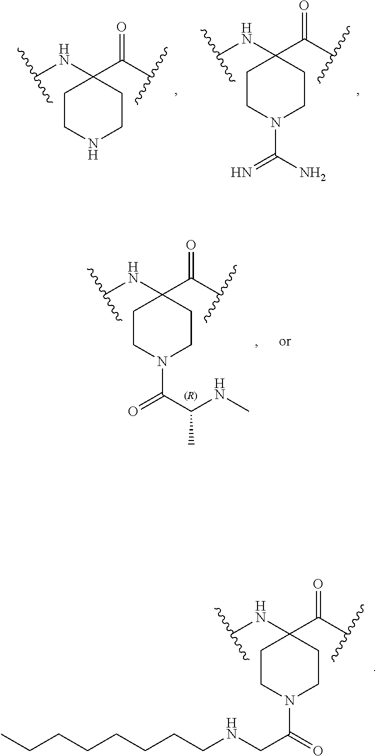

In some embodiments, X⁴ is

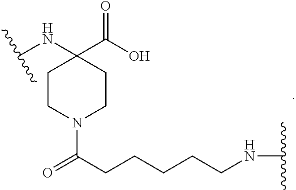 [SMILES: *NCCCCCC(=O)N1CCC(N*)(C(=O)O)CC1]

In some embodiments, R^{a}—X¹—X²—X³—X⁴— is:

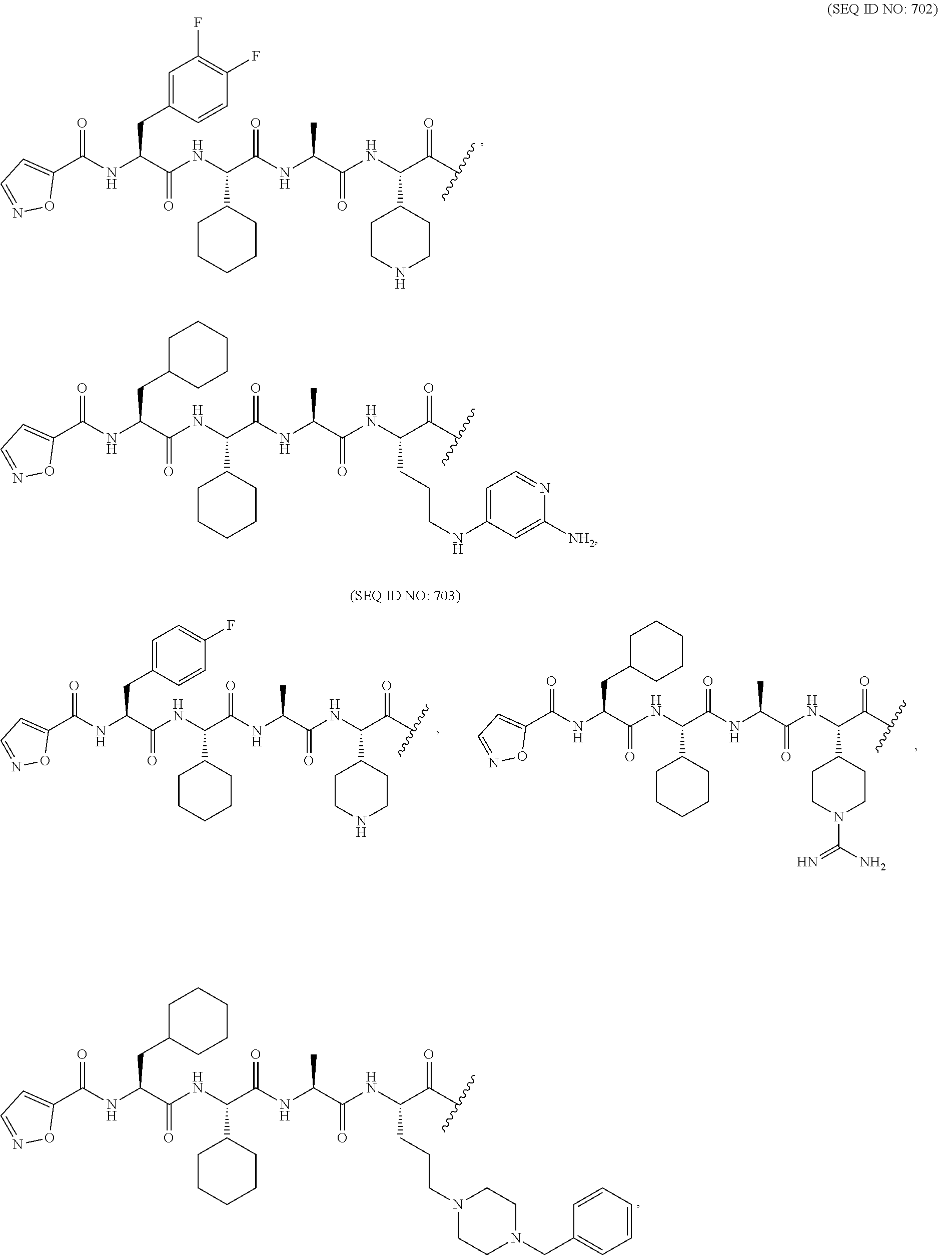

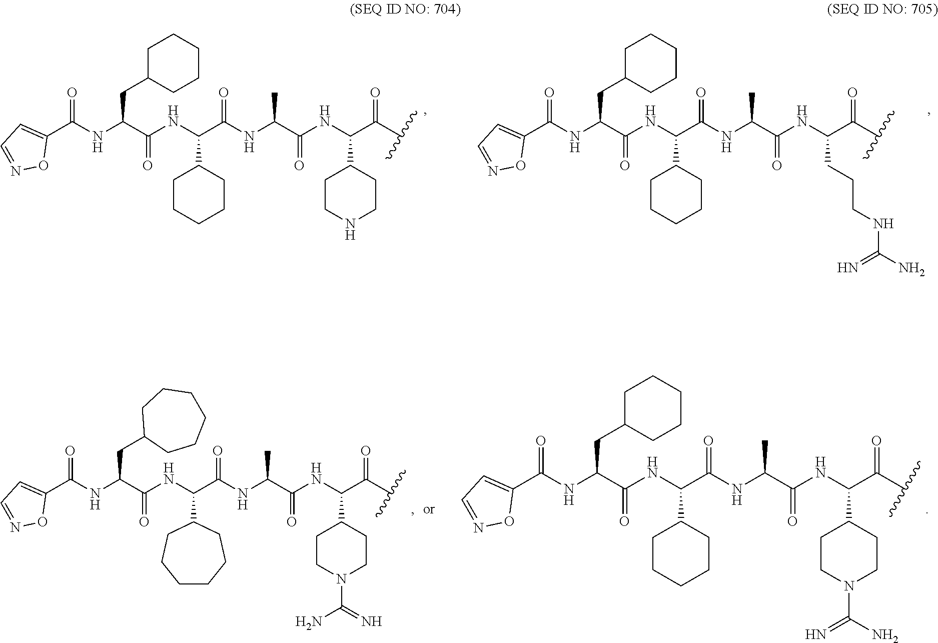 [SMILES: *C(=O)[C@@H](NC(=O)[C@H](C)NC(=O)[C@@H](NC(=O)[C@H](CC1CCCCC1)NC(=O)c1ccno1)C1CCCCC1)C1CCNCC1 ; *C(=O)[C@H](CCCNC(=N)N)NC(=O)[C@H](C)NC(=O)[C@@H](NC(=O)[C@H](CC1CCCCC1)NC(=O)c1ccno1)C1CCCCC1 ; *C(=O)[C@@H](NC(=O)[C@H](C)NC(=O)[C@@H](NC(=O)[C@H](CC1CCCCCC1)NC(=O)c1ccno1)C1CCCCCC1)C1CCN(C(=N)N)CC1 ; *C(=O)[C@@H](NC(=O)[C@H](C)NC(=O)[C@@H](NC(=O)[C@H](CC1CCCCC1)NC(=O)c1ccno1)C1CCCCC1)C1CCN(C(=N)N)CC1]

In some embodiments, X⁵ is -L⁵-, (S)-alanine (Ala), (S)-lysine (Lys), (S)-ornithine (Orn), or (S)-(4-fluorobenzamido)methylglycine. In some embodiments, X⁵ is -L⁵-. In some embodiments, X⁵ is (S)-lysine (Lys) or (S)-ornithine (Om). In some embodiments, X⁵ is (R)-lysine (Lys) or (R)-ornithine (Orn). In some embodiments, X⁵ is (S)-(4-fluorobenzamido)methylglycine.

In some embodiments, X⁵ is N6-palmitoyl-(S)-lysine, N6-(15-carboxypentadecanoyl)-(S)-Lys, N6-(16-oxo-16-((2-(N—N—N-trimethyl-amino)ethyl)amino)hexadecanoyl)-(S)-Lys, N6-(4-fluorobenzoyl)-(S)-lysine, (S)-β³-homolysine, (S)-3-amino-alanine (H-Dap-OH), 4-fluoro-(S)-phenylalanine (Phe(4-F)), 4-iodo-(S)-phenylalanine (Phe(4-I)), 4-nitro-(S)-phenylalanine (Phe(4-NO₂)), (S)-(4-fluorobenzamido)methylglycine, 4-aminomethylphenylalanine (Phe(4-CH₂NH₂)), (S)-7-hydroxy-1,2,3,4-tetrahydroisoquinoline-3-carboxylic acid, (S)-proline, (S)-hydroxyproline (Hyp), (S)-piperidine-2-carboxylic acid, (S)-isoindoline-1-carboxylic acid, or (S)-(4-(aminomethyl)-1H-1,2λ²,3λ²-triazol-1-yl)methyl)-glycine.

In some embodiments, X⁵ is alanine (ala), lysine (Lys), ornithine (Orn),

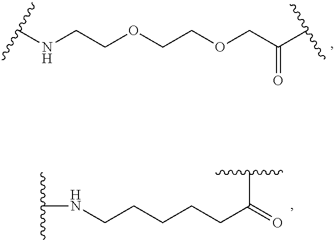 [SMILES: *NCCOCCOCC(*)=O ; *NCCCCCC(*)=O]

N6-(4-fluorobenzoyl)-Lys, β³-homolysine, 3-amino-alanine (H-Dap-OH), 4-nitrophenylalanine (Phe(4-NO₂)), or (4-fluorobenzamido)methylglycine. In some embodiments, X⁵ is absent or

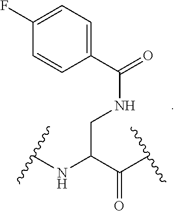 [SMILES: *NC(CNC(=O)c1ccc(F)cc1)C(*)=O]

In some embodiments, X¹ is

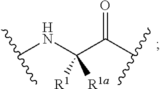

R¹ is -L¹-R^{1b};
L is —CH₂—;
R^{1b} is cyclohexyl, cycloheptyl, or 4-fluorophenyl;
R^{1a} is hydrogen;
X² is

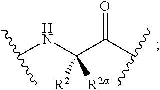

R² is -L²-R^{2b};
L² is absent;
R^{2b} is cyclohexyl, cycloheptyl, 2,3-dihydro-1H-inden-2-yl, or [1,1′-biphenyl]-4-yl;
R^{2a} is hydrogen;
X³ is (S)-alanine (Ala) or (S)-norleucine (Nle);
X⁴ is (S)-arginine (Arg), (S)-ornithine (Orn), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-3-(6-aminopyridin-3-yl)alanine ((3-Pal(6-NH₂)), or (S)-2-((2-aminopyridin-4-yl)-λ²-azaneyl)ethyl alanine; and
X⁵ is -L⁵-, alanine (Ala), lysine (Lys), or ornithine (Orn).

In some embodiments, X⁶ is absent. In some embodiments, X⁶ is -L⁶-. In some embodiments, X⁶ is (S)-lysine (Lys) or (S)-ornithine (Orn). In some embodiments, X⁶ is (R)-lysine (Lys) or (R)-ornithine (Orn).

In some embodiments, X⁶ is absent, lysine (Lys), ornithine (Orn),

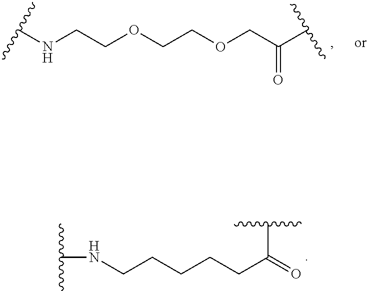 [SMILES: *NCCOCCOCC(*)=O ; *NCCCCCC(*)=O]

In some embodiments, X⁶ is

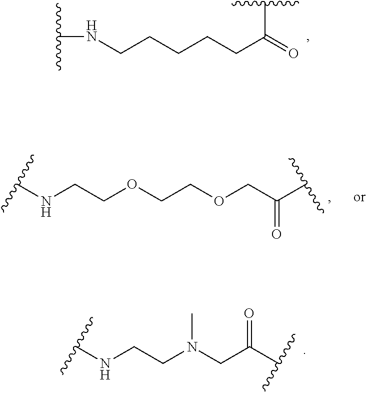

In some embodiments, X⁶ is 4-amino-1-(methylglycyl)piperidine-4-carboxylic acid, or 4-amino-1-(N-carbamimidoyl-N-methylglycyl)piperidine-4-carboxylic acid. In some embodiments, X⁶ is (4-fluorobenzamido)methylglycine. In some embodiments, X⁶ is N6-(4-fluorobenzoyl)-Lys.

In some embodiments, X⁷ is absent, lysine (Lys), ornithine (Orn), or

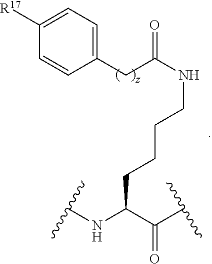

In some embodiments, X⁷ is absent. In some embodiments, X⁷ is (S)-lysine (Lys) or (S)-ornithine (Orn). In some embodiments, X⁷ is glutamic acid (Glu). In some embodiments, X⁷ is norleucine (Nle). In some embodiments, X⁷ is

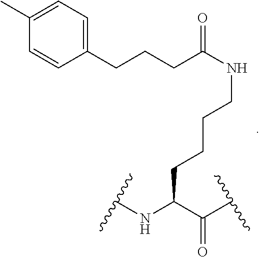 [SMILES: *N[C@@H](CCCCNC(=O)CCCc1ccc(C)cc1)C(*)=O]

In some embodiments, X⁷ is

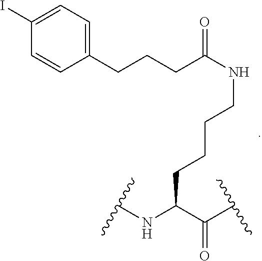 [SMILES: *N[C@@H](CCCCNC(=O)CCCc1ccc(I)cc1)C(*)=O]

In some embodiments, R^{a} is —C(═O)—R¹⁰, wherein R¹⁰ is a heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl, indolizinyl, indolyl, benzofuranyl, benzothiophenyl, indazolyl, benzimidazolyl, purinyl, quinolizinyl, quinolinyl, isoquinolinyl, cinnolinyl, phthalazinyl, quinazolinyl, quinoxalinyl, 1,8-naphthyridinyl, 1,7-naphthyridinyl, 1,6-naphthyridinyl, 1,5-naphthyridinyl, pteridinyl, and tetrazolo[1,5-a]pyridinyl; and wherein R¹⁰ is optionally substituted with I to 3 substituents that are each independently selected from the group consisting of halo, —C₁-C₆ alkyl, alkoxy, —OH, —NH₂, —NH—C₁-C₆ alkylene-R^{II}, and —O—C₁-C₆ alkylene-R¹¹. In some embodiments, R^{a} is —C(═O)—R¹⁰, wherein R¹⁰ is a monocyclic heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl; and wherein R¹⁰ is optionally substituted with 1 to 3 substituents that are each independently selected from the group consisting of halo, —C₁-C₆ alkyl, alkoxy, —OH, —NH₂, —NH—C₁-C₆ alkylene-R¹¹, and —O—C₁-C₆ alkylene-R¹¹. In some embodiments, R^{a} is —C(═O)—R¹⁰, wherein R¹⁰ is a monocyclic heteroaryl selected from the group consisting of pyridinyl, pyrimidinyl, pyrazinyl, and pyridazinyl; and wherein R¹⁰ is optionally substituted with —NH—C₁-C₆ alkylene-R¹, and —O—C₁-C₆ alkylene-R¹. In some embodiments, R^{a} is —C(═O)—R¹⁰, wherein R¹⁰ is a phenyl or naphthyl; and wherein R¹⁰ is optionally substituted with 1 to 3 substituents that are each independently selected from the group consisting of halo, C₁-C₆ alkyl, alkoxy, —OH, —NH, —NH—C₁-C₆alkylene-R¹¹, and —O—C₁-C₆ alkylene-R¹¹. In some embodiments, R^{a} is —C(═O)—R¹⁰, wherein R¹⁰ is a partially unsaturated bicyclic heterocyclic ring; wherein any saturated carbon of R¹⁰ is optionally substituted with oxo (═O); and wherein R¹⁰ is optionally substituted with 1 to 3 substituents that are each independently selected from the group consisting of halo, C₁-C₆ alkyl, alkoxy, —OH, and —NH₂. In some embodiments, R^{a} is —C(═O)—R¹⁰, wherein R¹⁰ is unsubstituted isoxazolyl.

In some embodiments, R^{a} is

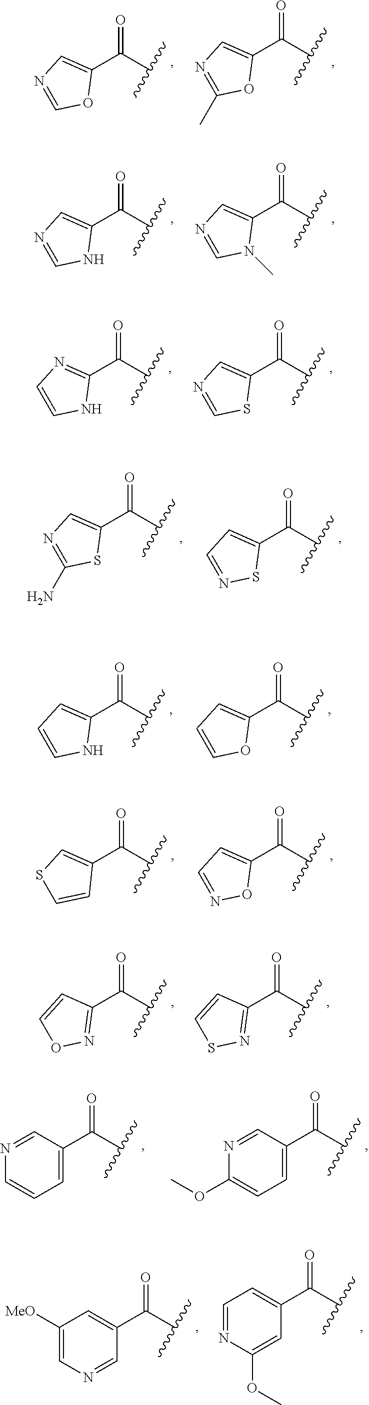

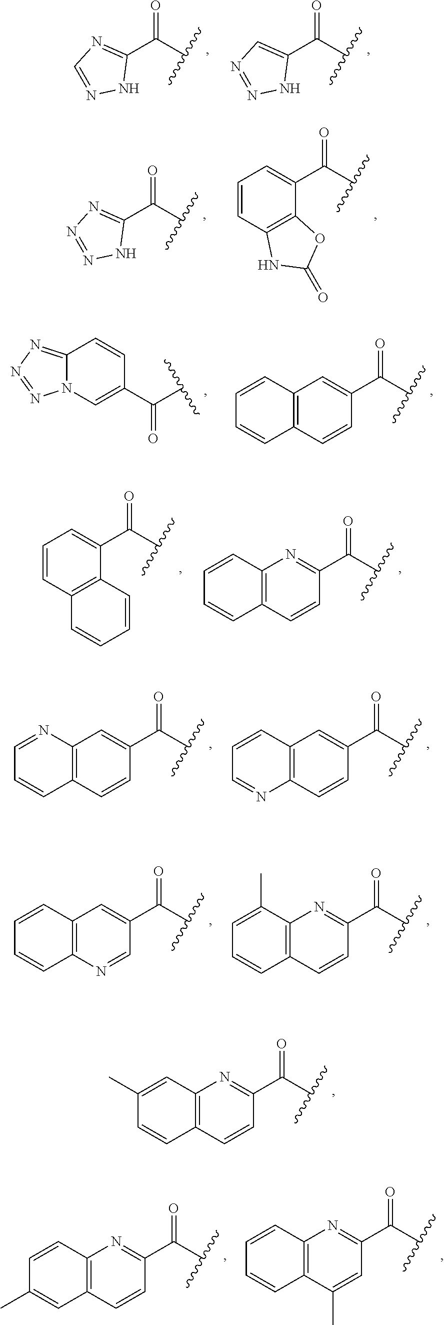

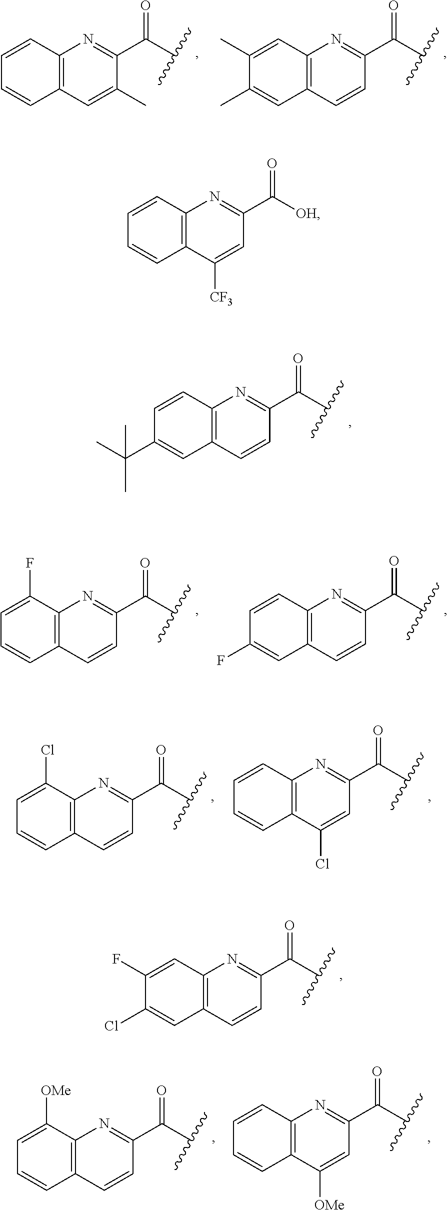

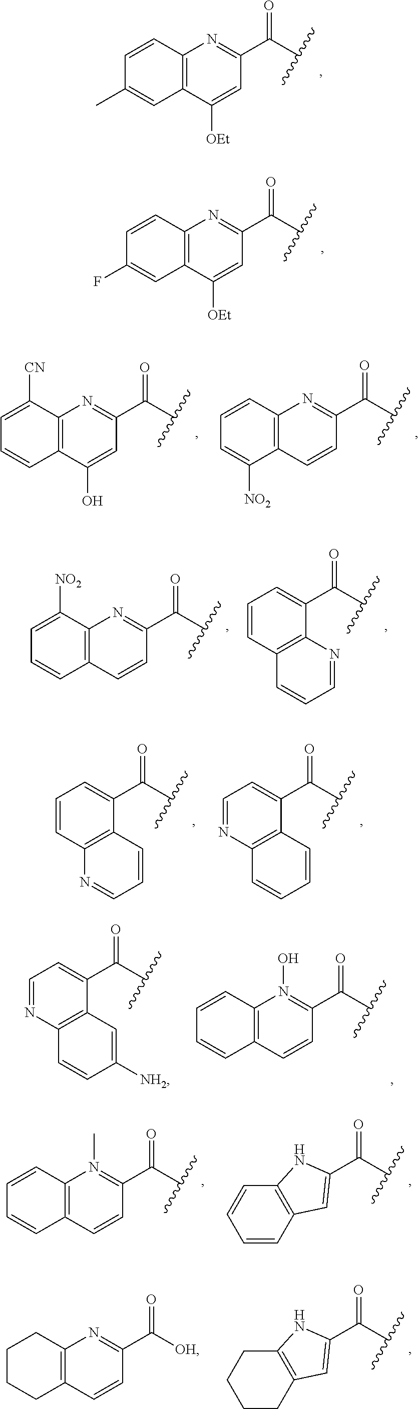

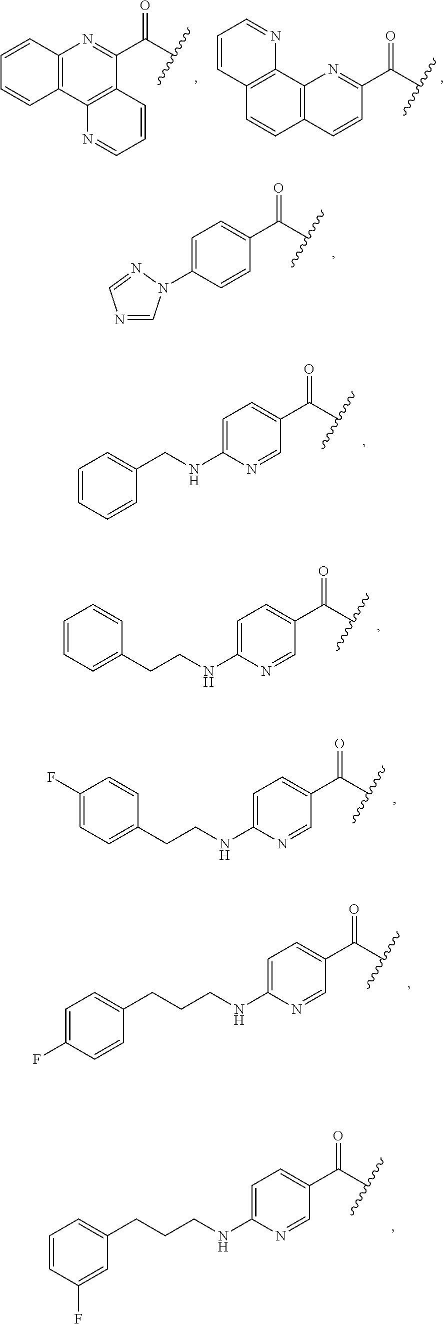

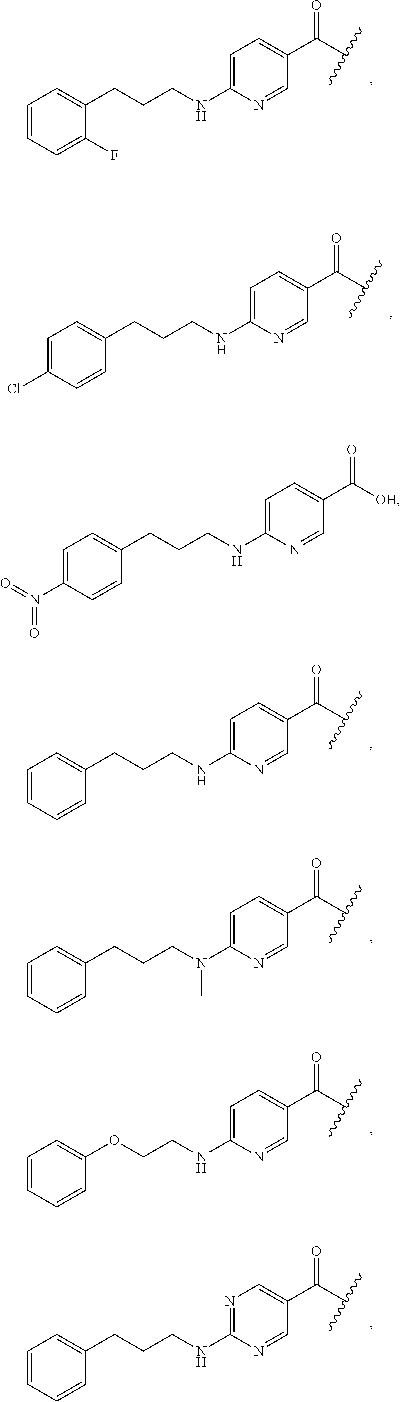

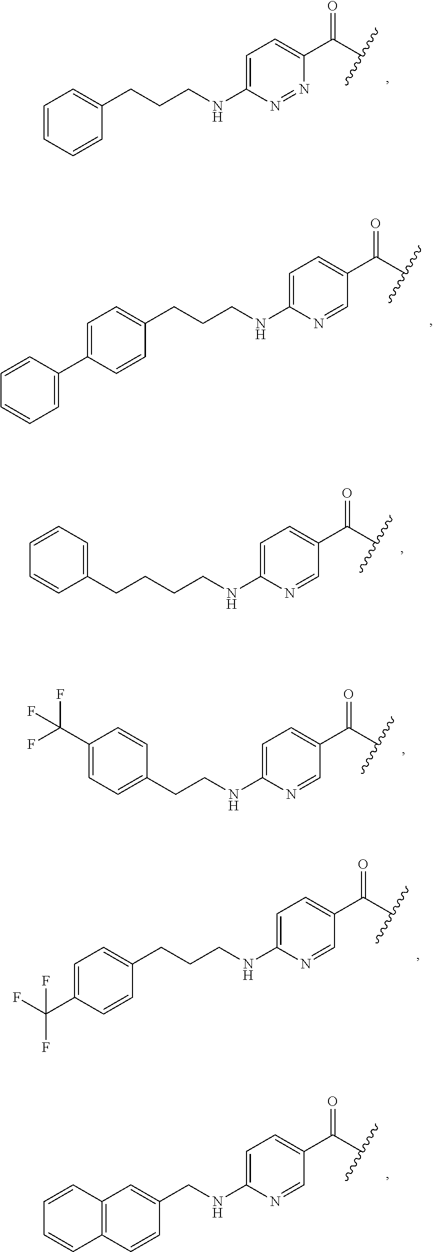 [SMILES: *C(=O)c1ccc(NCCCc2ccccc2)nn1 ; *C(=O)c1ccc(NCCCc2ccc(-c3ccccc3)cc2)nc1 ; *C(=O)c1ccc(NCCCCc2ccccc2)nc1 ; *C(=O)c1ccc(NCCc2ccc(C(F)(F)F)cc2)nc1 ; *C(=O)c1ccc(NCCCc2ccc(C(F)(F)F)cc2)nc1 ; *C(=O)c1ccc(NCc2ccc3ccccc3c2)nc1]

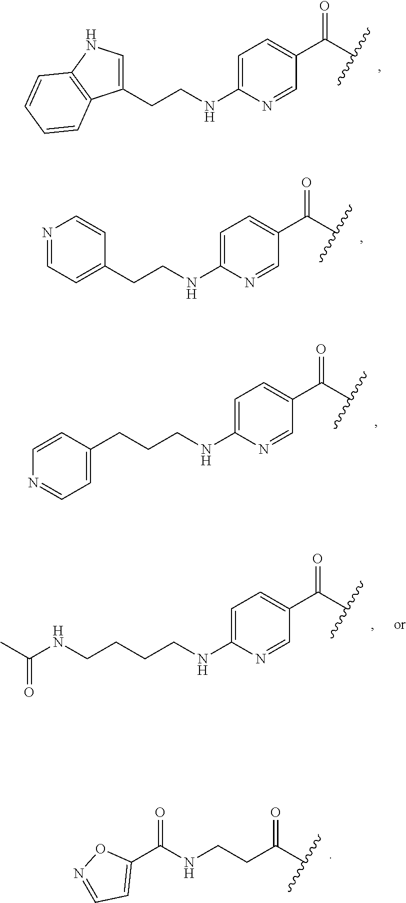 [SMILES: *C(=O)c1ccc(NCCc2c[nH]c3ccccc23)nc1 ; *C(=O)c1ccc(NCCc2ccncc2)nc1 ; *C(=O)c1ccc(NCCCc2ccncc2)nc1 ; *C(=O)c1ccc(NCCCCNC(C)=O)nc1 ; *C(=O)CCNC(=O)c1ccno1]

In some embodiments, R^{a} is

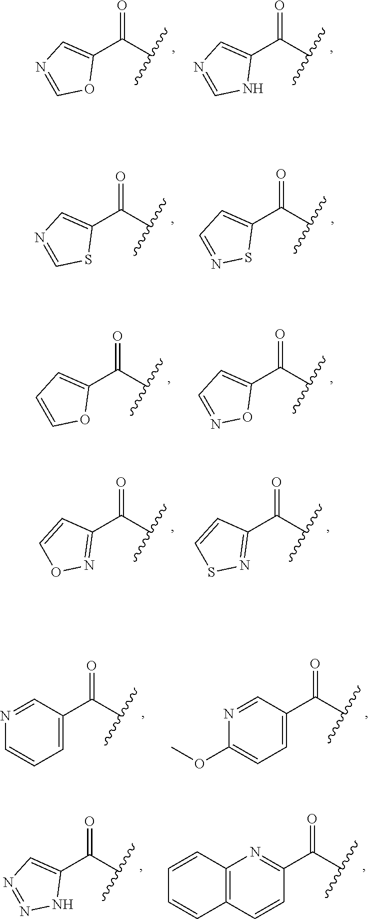

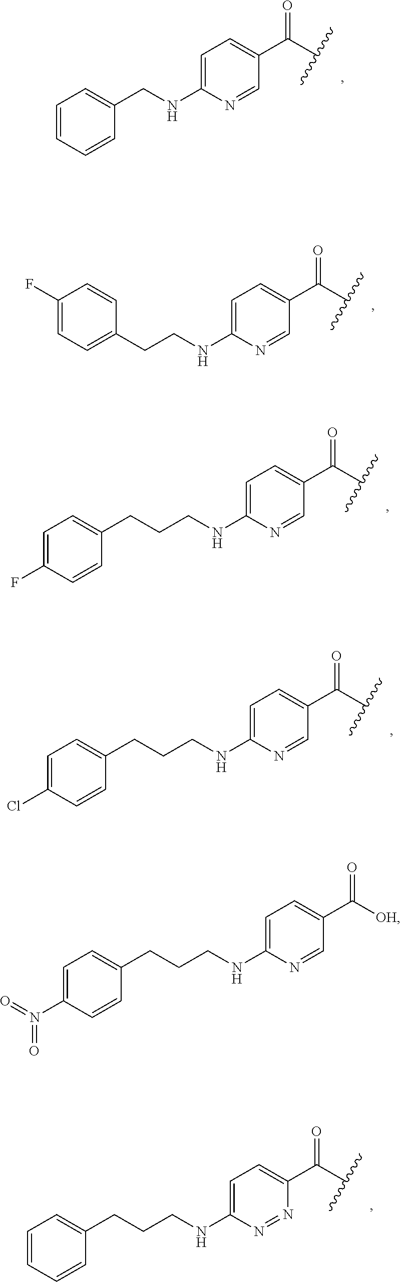 [SMILES: *C(=O)c1ccc(NCc2ccccc2)nc1 ; *C(=O)c1ccc(NCCc2ccc(F)cc2)nc1 ; *C(=O)c1ccc(NCCCc2ccc(F)cc2)nc1 ; *C(=O)c1ccc(NCCCc2ccc(Cl)cc2)nc1 ; O=C(O)c1ccc(NCCCc2ccc([N+](=O)[O-])cc2)nc1 ; *C(=O)c1ccc(NCCCc2ccccc2)nn1]

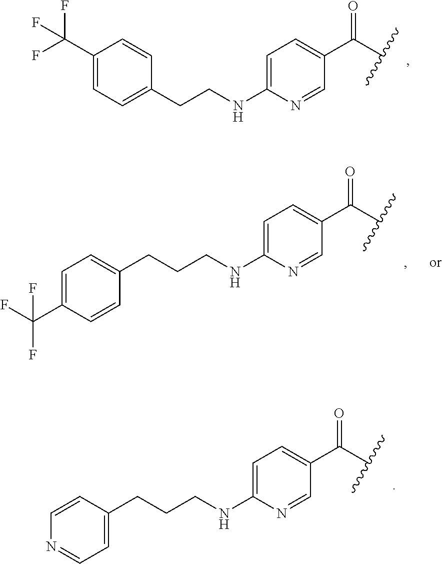 [SMILES: *C(=O)c1ccc(NCCc2ccc(C(F)(F)F)cc2)nc1 ; *C(=O)c1ccc(NCCCc2ccc(C(F)(F)F)cc2)nc1 ; *C(=O)c1ccc(NCCCc2ccncc2)nc1]

In some embodiments, R^{a} is

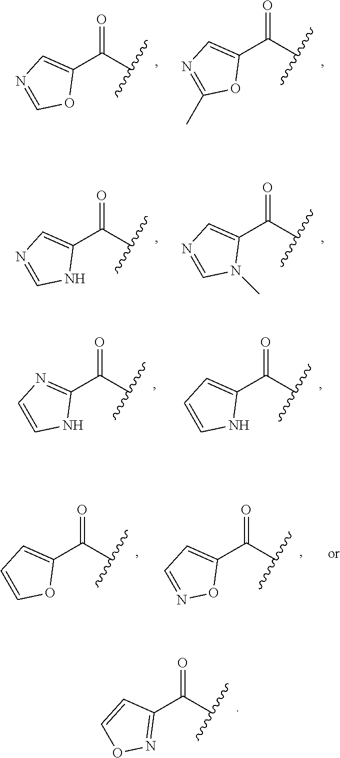

In some embodiments, R^{a} is

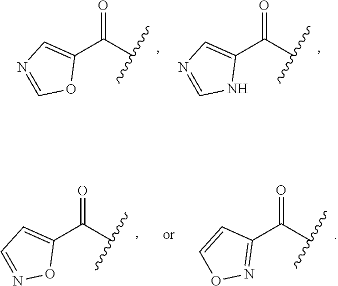 [SMILES: *C(=O)c1cnco1 ; *C(=O)c1cnc[nH]1 ; *C(=O)c1ccno1 ; *C(=O)c1ccon1]

In some embodiments, R^{a} is

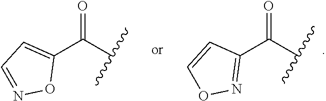 [SMILES: *C(=O)c1ccno1 ; *C(=O)c1ccon1]

In some embodiments, -L⁵-, -L⁶-, -L⁷- are each independently selected from the group consisting of: —R¹⁵—C₁-C₆ alkyl-C(═O)—, —NR ¹⁵—(CH₂CH₂O)_{y}—C₁-C₆ alkyl-C(═O)—, —NR¹⁵(CH₂CH₂NR¹⁶)_{y}—C₁-C₆ alkyl-C(═O)—, —NR¹⁵—C₁-C₆ alkyl-(substituted or unsubstituted heterocycloalkyl)-C(═O)—, —NR¹⁵-(substituted or unsubstituted heterocycloalkyl)-C₁-C₆ alkyl-C(═O)—, —NR¹⁵—C₁-C₆ alkyl-(substituted or unsubstituted cyclohexyl)-C(═O)—, and —NR¹⁵-(substituted or unsubstituted cyclohexyl)-C₁-C₆ alkyl-C(═O)—; R¹⁵ and R¹⁶ are each independently selected from H or —C₁-C₄ alkyl; and y is 1, 2, 3, 4, 5, or 6.

In some embodiments, -L⁵-, -L⁶-, or -L⁷- are each independently selected from the group consisting of: —NH—C₁-C₆ alkylene-C(═O)—, —NH—(CH₂CH₂O)_{y}—C₁-C₆ alkylene-C(═O)—, —NH(CH₂CH₂NR¹⁶)_{y}—C₁-C₆alkylene-C(═O)—, —NH—C₁-C₆alkylene-(unsubstituted piperidinylene)-C(═O)—, —NH-(unsubstituted piperidinylene)-C₁-C₆ alkylene-C(═O)—, —NH—C₁-C₆ alkylene-(unsubstituted cyclohexylene)-C(═O)—, and —NH-(unsubstituted cyclohexylene)-C₁-C₆ alkylene-C(═O)—.

In some embodiments, -L⁵-, -L⁶-, or -L⁷- are each independently selected from the group consisting of:

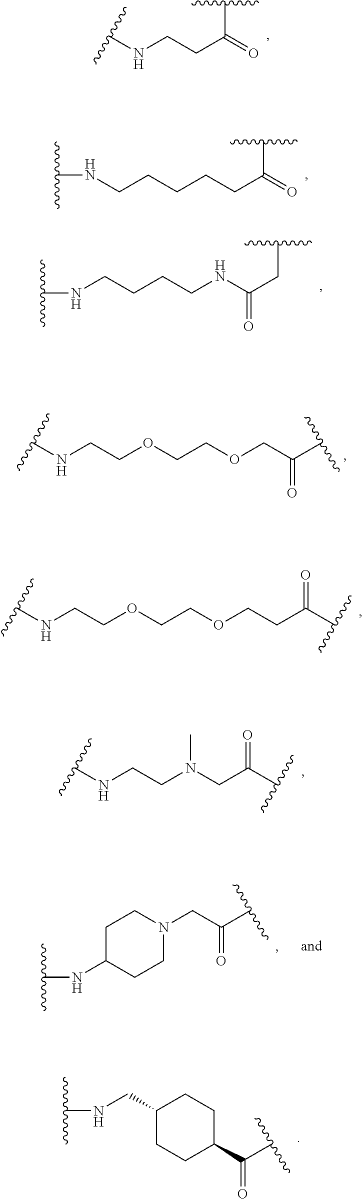

In some embodiments, the compound of Formula (VII) is any one of compounds 1 to 210, or a pharmaceutically acceptable salt thereof.

In some embodiments, the compound of Formula (VII), or a pharmaceutically acceptable salt thereof, has the following chemical structure, or a pharmaceutically acceptable salt thereof:

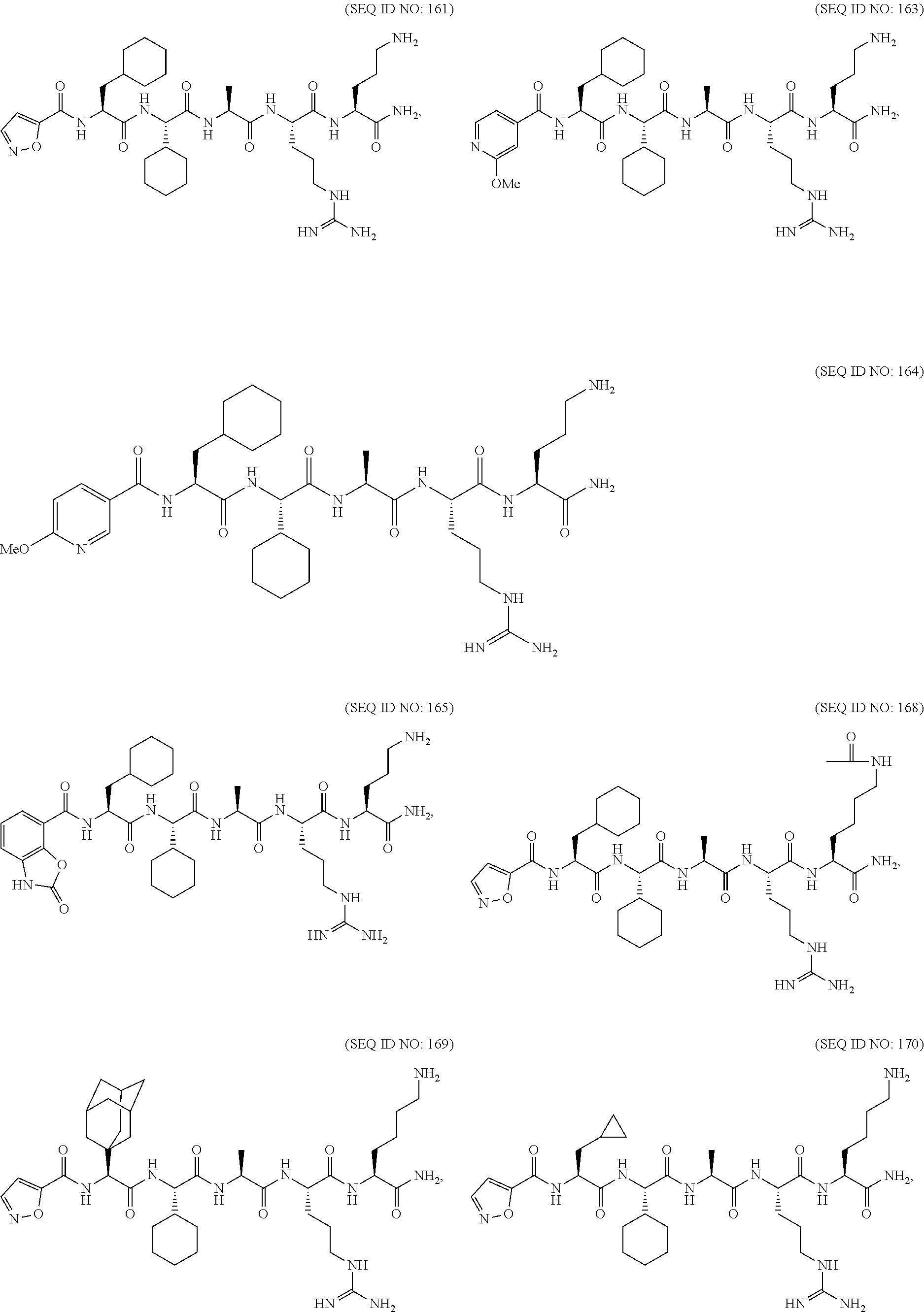

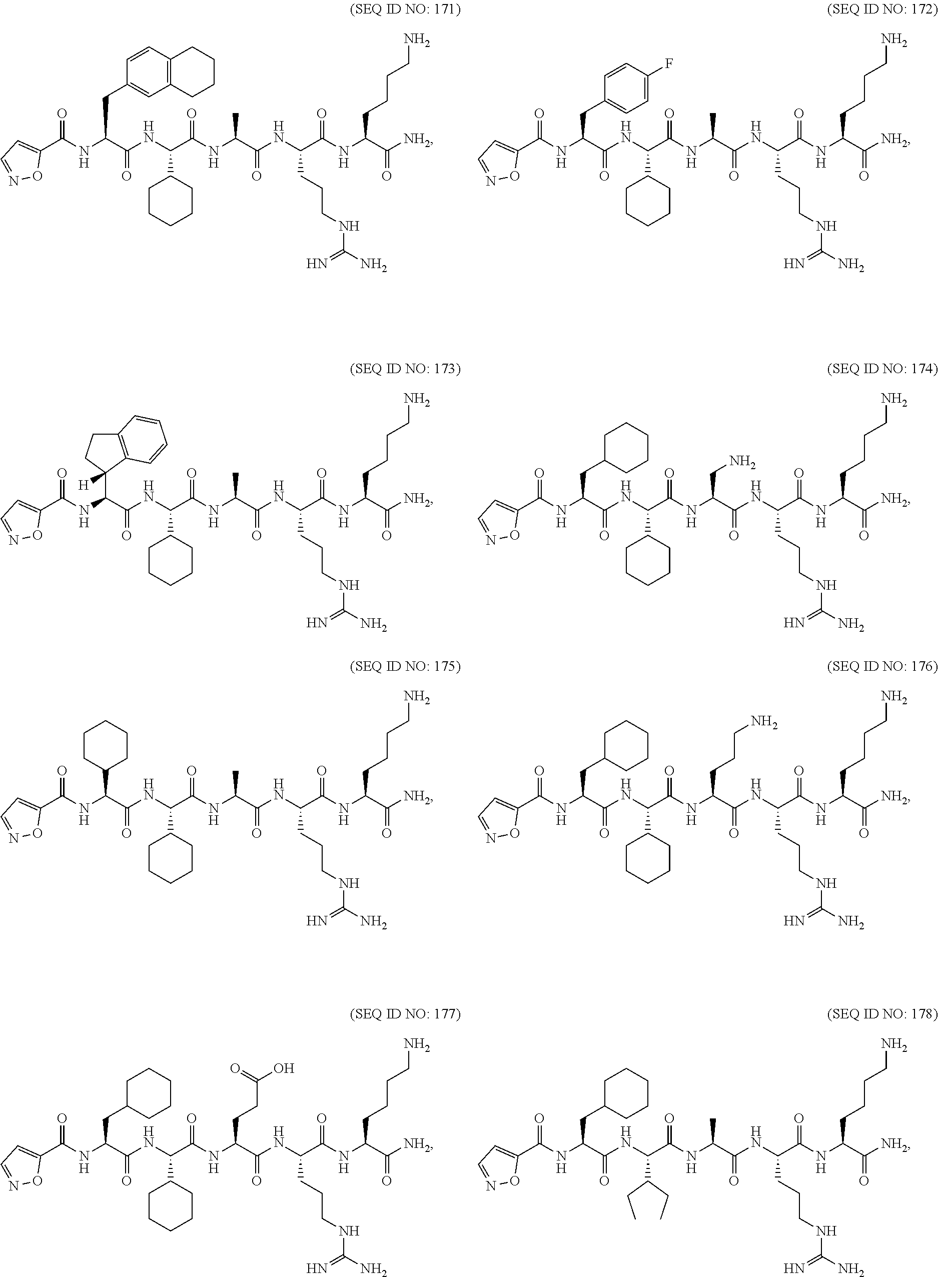

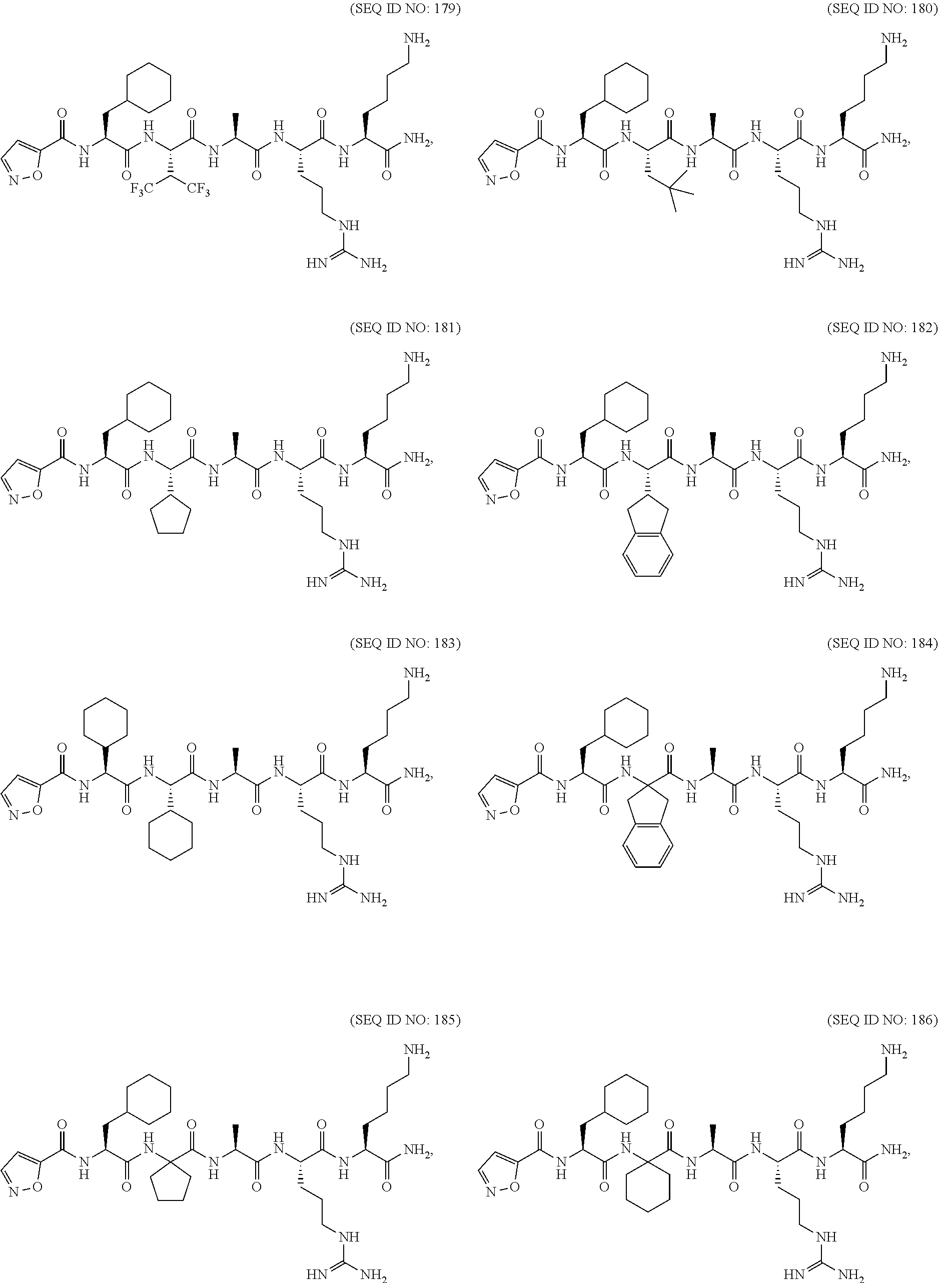

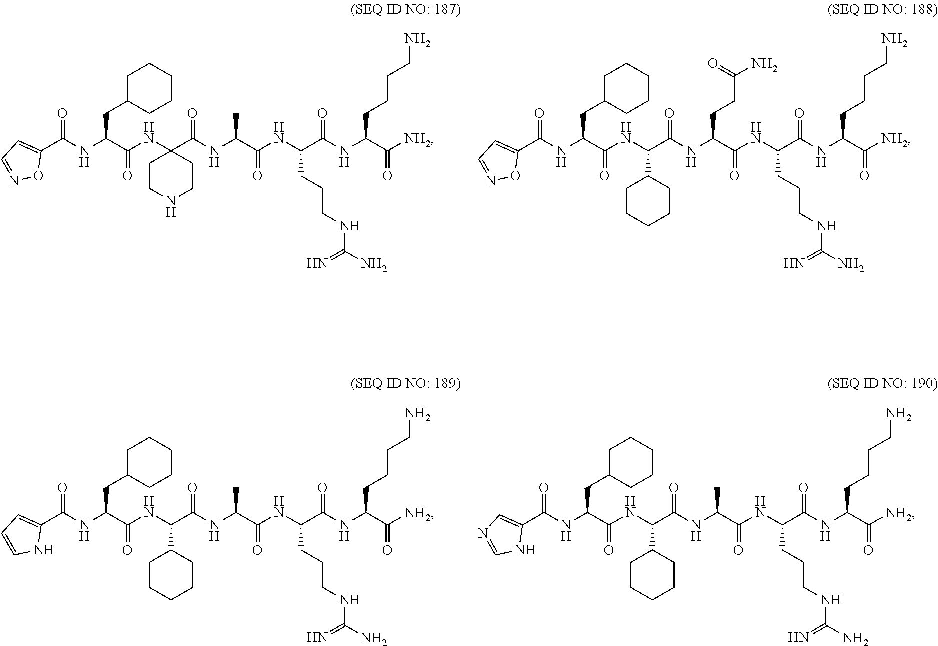 [SMILES: C[C@H](NC(=O)C1(NC(=O)[C@H](CC2CCCCC2)NC(=O)c2ccno2)CCNCC1)C(=O)N[C@@H](CCCNC(=N)N)C(=O)N[C@@H](CCCCN)C(N)=O ; N=C(N)NCCC[C@H](NC(=O)[C@H](CCC(N)=O)NC(=O)[C@@H](NC(=O)[C@H](CC1CCCCC1)NC(=O)c1ccno1)C1CCCCC1)C(=O)N[C@@H](CCCCN)C(N)=O ; C[C@H](NC(=O)[C@@H](NC(=O)[C@H](CC1CCCCC1)NC(=O)c1ccc[nH]1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1cnc[nH]1)C1CCCCC1)C(=O)N[C@@H](CCCNC(=N)N)C(=O)N[C@@H](CCCCN)C(N)=O]

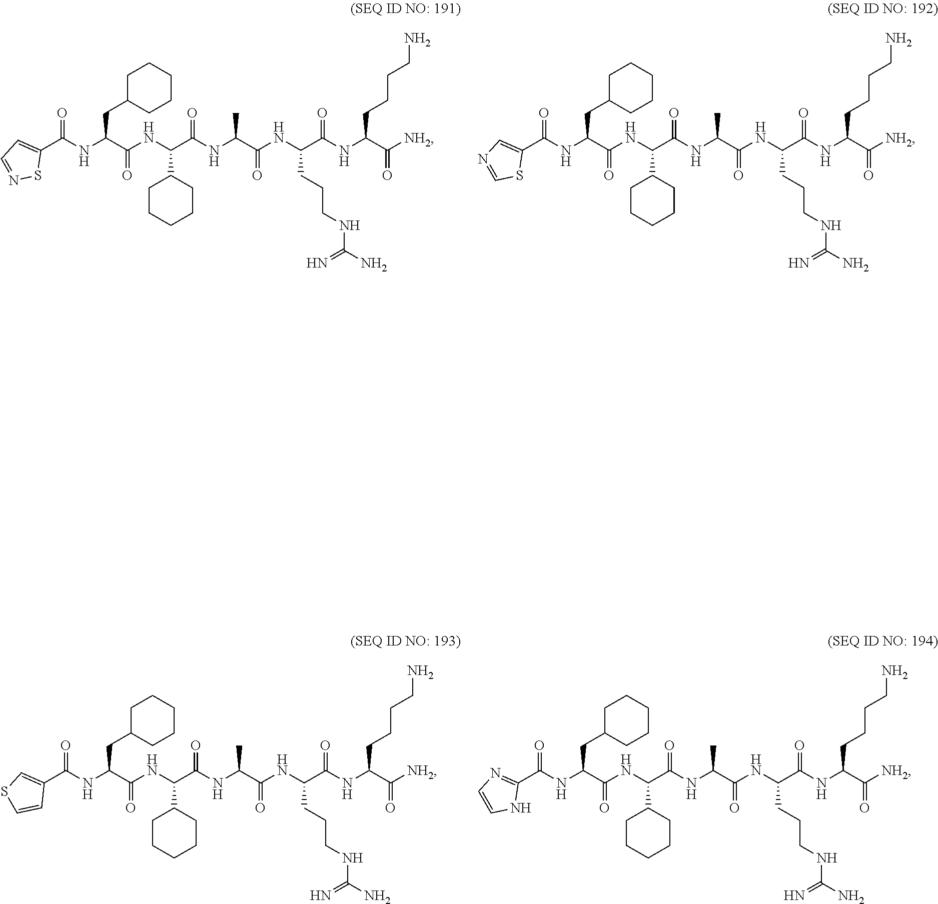 [SMILES: C[C@H](NC(=O)[C@@H](NC(=O)[C@H](CC1CCCCC1)NC(=O)c1ccns1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1cncs1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccsc1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ncc[nH]1)C1CCCCC1)C(=O)N[C@@H](CCCNC(=N)N)C(=O)N[C@@H](CCCCN)C(N)=O]

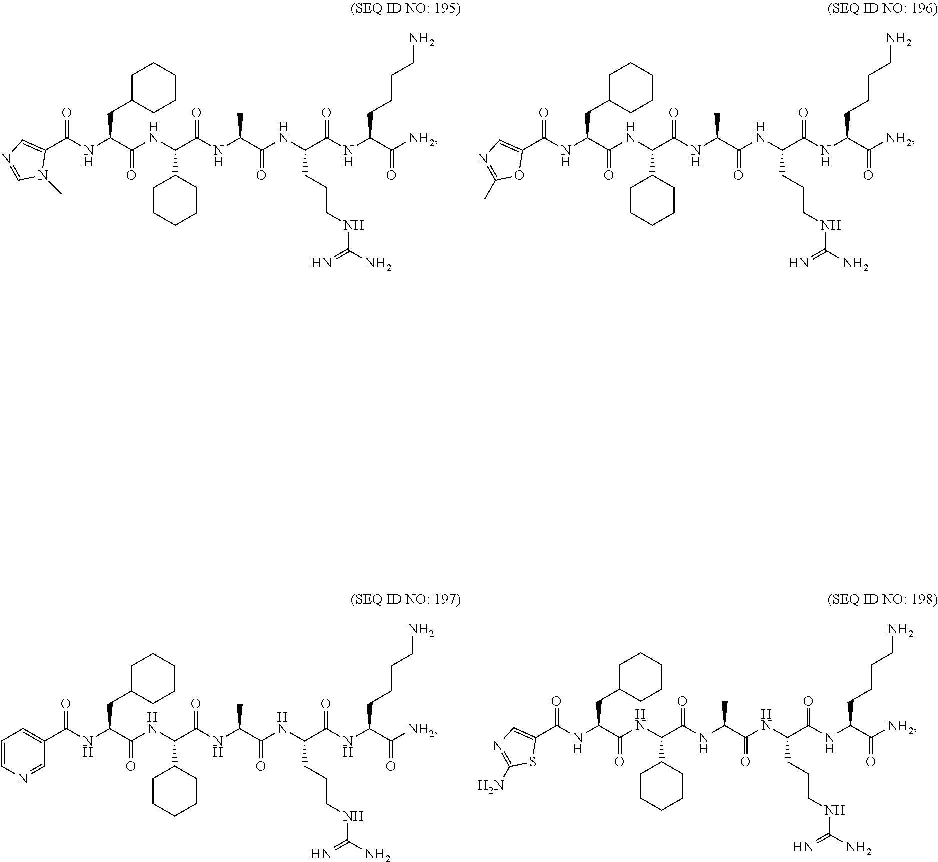 [SMILES: C[C@H](NC(=O)[C@@H](NC(=O)[C@H](CC1CCCCC1)NC(=O)c1cncn1C)C1CCCCC1)C(=O)N[C@@H](CCCNC(=N)N)C(=O)N[C@@H](CCCCN)C(N)=O ; Cc1ncc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)o1 ; C[C@H](NC(=O)[C@@H](NC(=O)[C@H](CC1CCCCC1)NC(=O)c1cccnc1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1cnc(N)s1)C1CCCCC1)C(=O)N[C@@H](CCCNC(=N)N)C(=O)N[C@@H](CCCCN)C(N)=O]

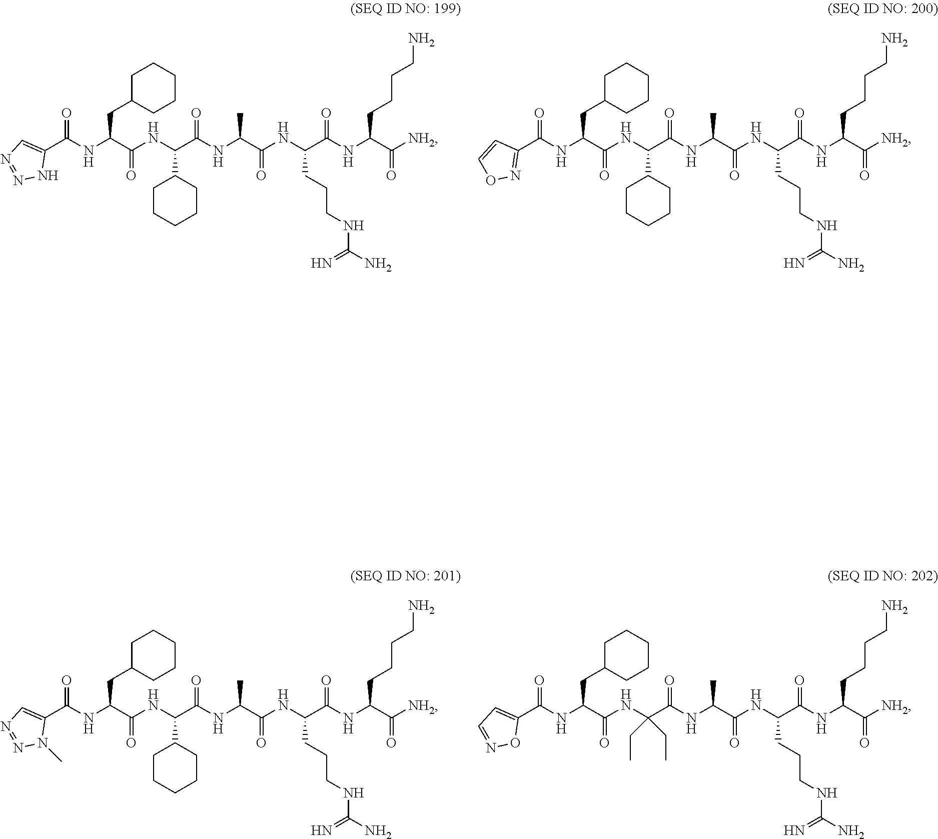 [SMILES: C[C@H](NC(=O)[C@@H](NC(=O)[C@H](CC1CCCCC1)NC(=O)c1cnn[nH]1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccon1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1cnnn1C)C1CCCCC1)C(=O)N[C@@H](CCCNC(=N)N)C(=O)N[C@@H](CCCCN)C(N)=O ; CCC(CC)(NC(=O)[C@H](CC1CCCCC1)NC(=O)c1ccno1)C(=O)N[C@@H](C)C(=O)N[C@@H](CCCNC(=N)N)C(=O)N[C@@H](CCCCN)C(N)=O]

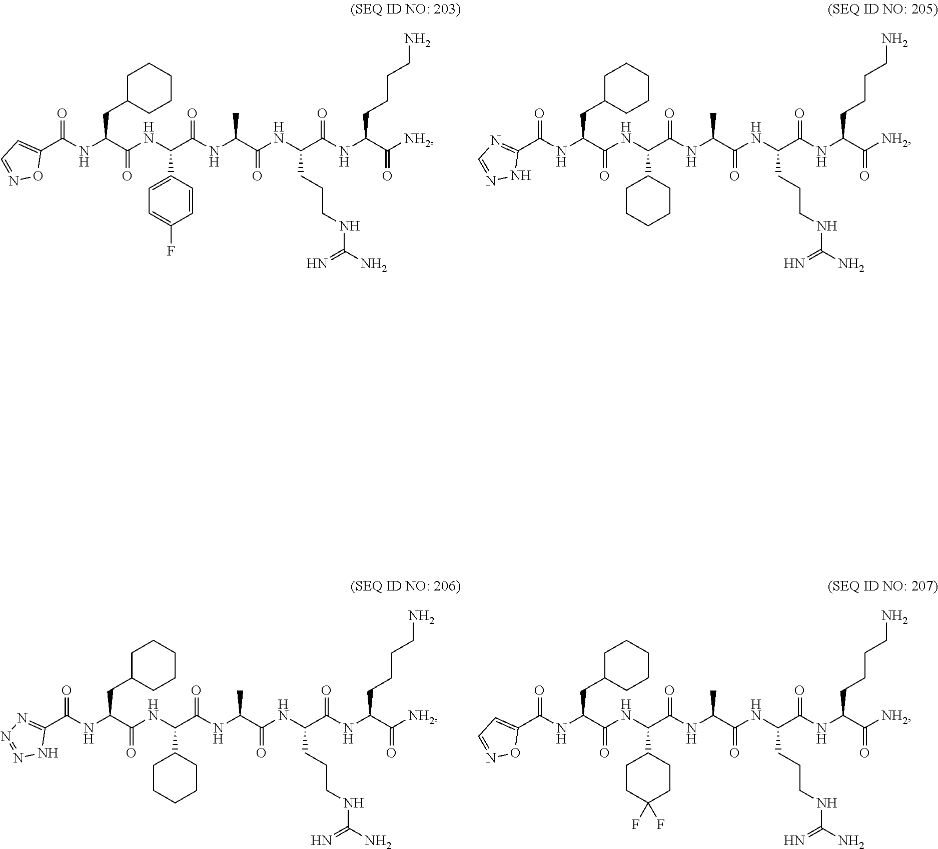 [SMILES: C[C@H](NC(=O)[C@@H](NC(=O)[C@H](CC1CCCCC1)NC(=O)c1ccno1)c1ccc(F)cc1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ncn[nH]1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1nnn[nH]1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccno1)C1CCC(F)(F)CC1)C(=O)N[C@@H](CCCNC(=N)N)C(=O)N[C@@H](CCCCN)C(N)=O]

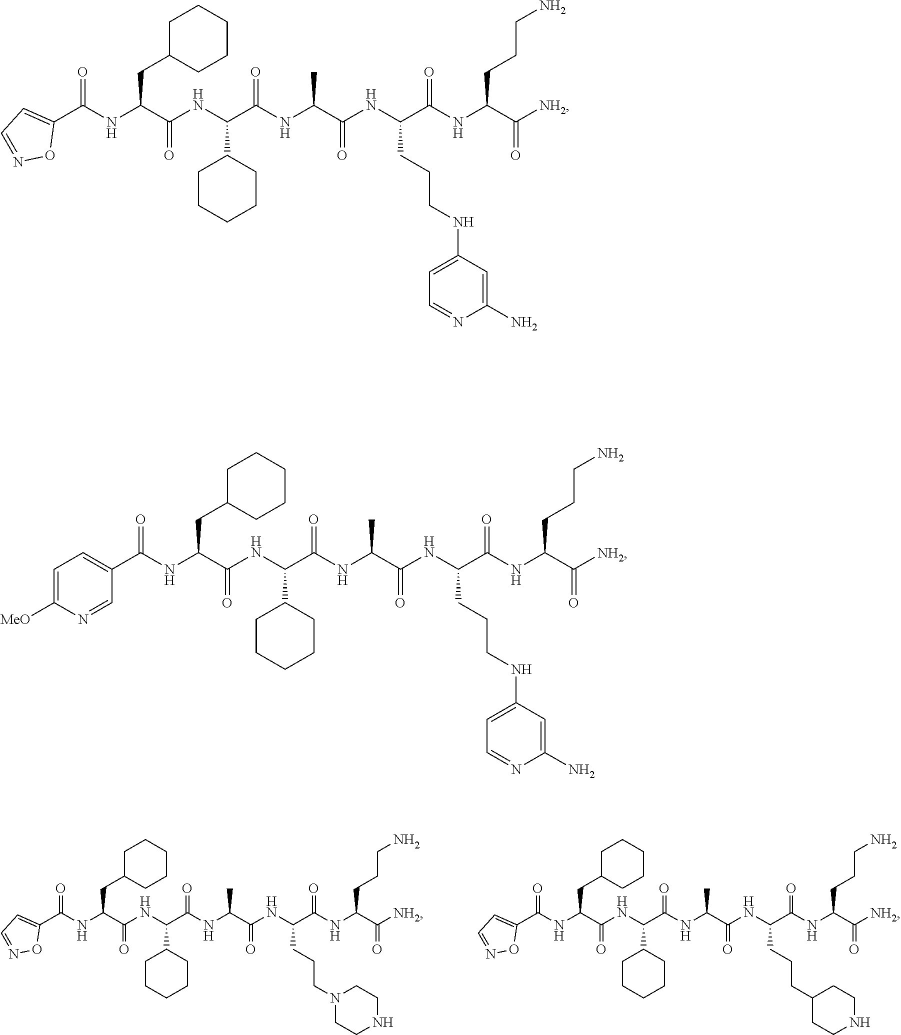 [SMILES: C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N[C@@H](CCCN)C(N)=O ; COc1ccc(C(=O)N[C@@H](CC2CCCCC2)C(=O)N[C@H](C(=O)N[C@@H](C)C(=O)N[C@@H](CCCNc2ccnc(N)c2)C(=O)N[C@@H](CCCN)C(N)=O)C2CCCCC2)cn1 ; C[C@H](NC(=O)[C@@H](NC(=O)[C@H](CC1CCCCC1)NC(=O)c1ccno1)C1CCCCC1)C(=O)N[C@@H](CCCN1CCNCC1)C(=O)N[C@@H](CCCN)C(N)=O ; C[C@H](NC(=O)[C@@H](NC(=O)[C@H](CC1CCCCC1)NC(=O)c1ccno1)C1CCCCC1)C(=O)N[C@@H](CCCC1CCNCC1)C(=O)N[C@@H](CCCN)C(N)=O]

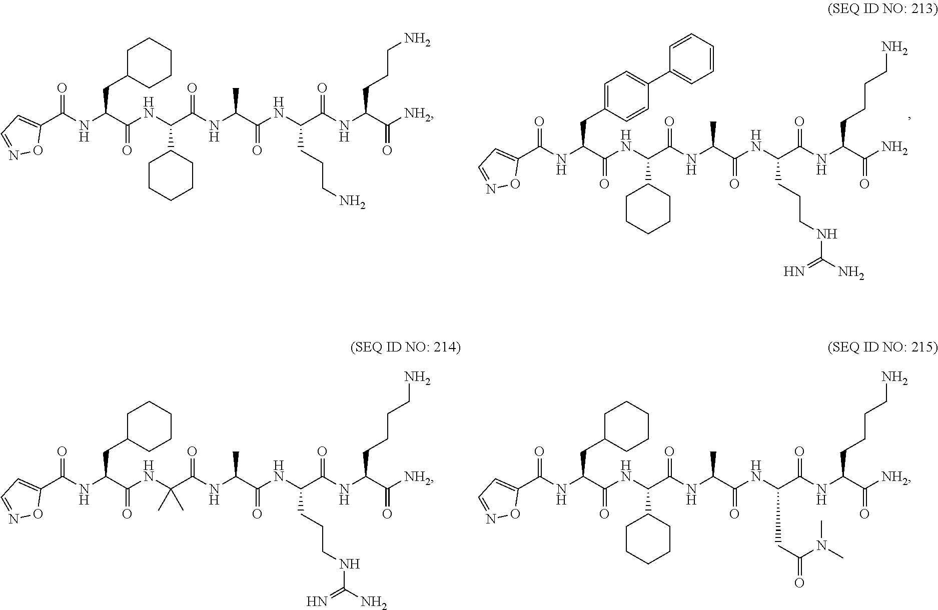 [SMILES: C[C@H](NC(=O)[C@@H](NC(=O)[C@H](CC1CCCCC1)NC(=O)c1ccno1)C1CCCCC1)C(=O)N[C@@H](CCCN)C(=O)N[C@@H](CCCN)C(N)=O ; C[C@H](NC(=O)[C@@H](NC(=O)[C@H](Cc1ccc(-c2ccccc2)cc1)NC(=O)c1ccno1)C1CCCCC1)C(=O)N[C@@H](CCCNC(=N)N)C(=O)N[C@@H](CCCCN)C(N)=O ; C[C@H](NC(=O)C(C)(C)NC(=O)[C@H](CC1CCCCC1)NC(=O)c1ccno1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccno1)C1CCCCC1)C(=O)N[C@@H](CC(=O)N(C)C)C(=O)N[C@@H](CCCCN)C(N)=O]

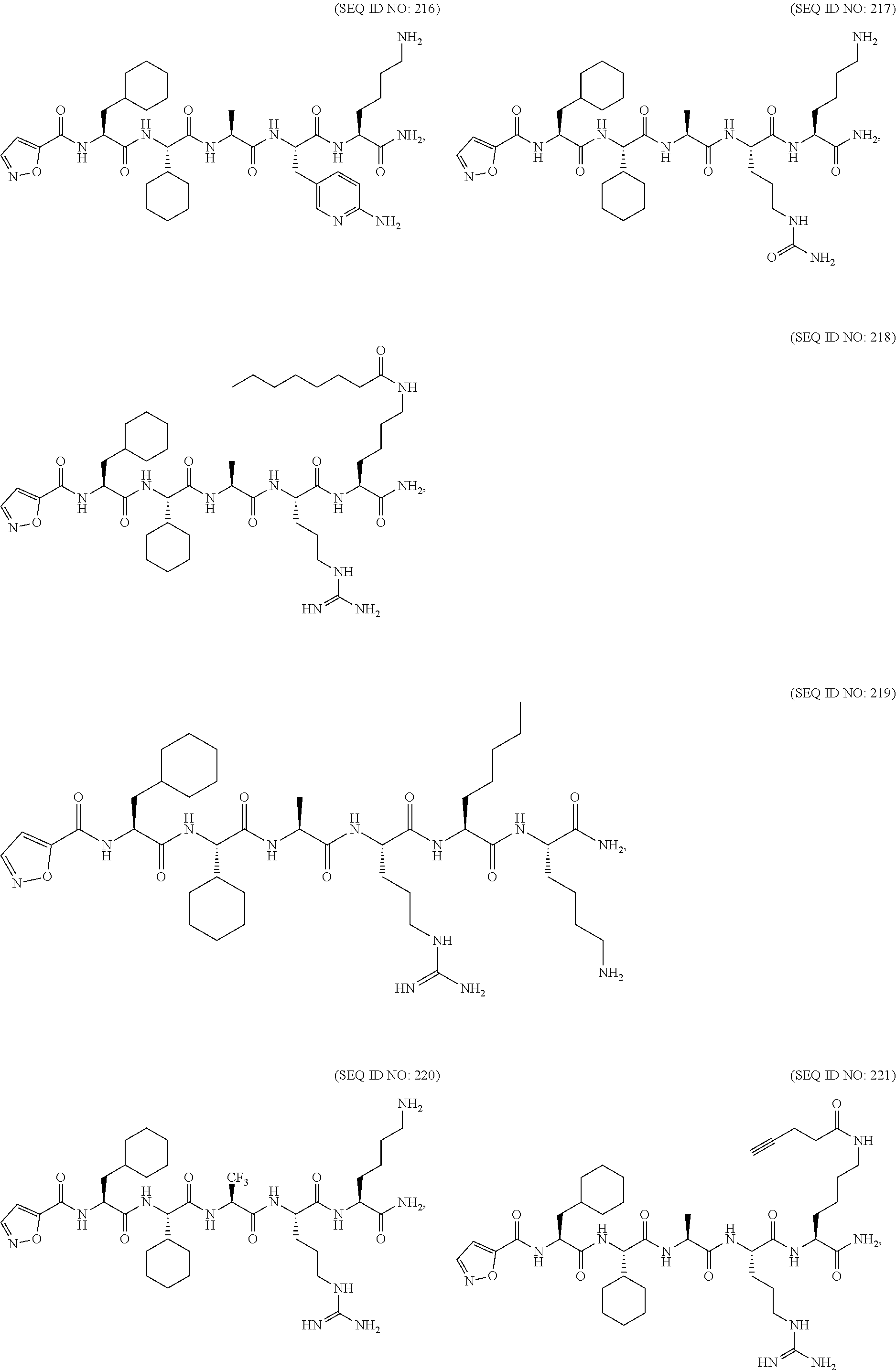

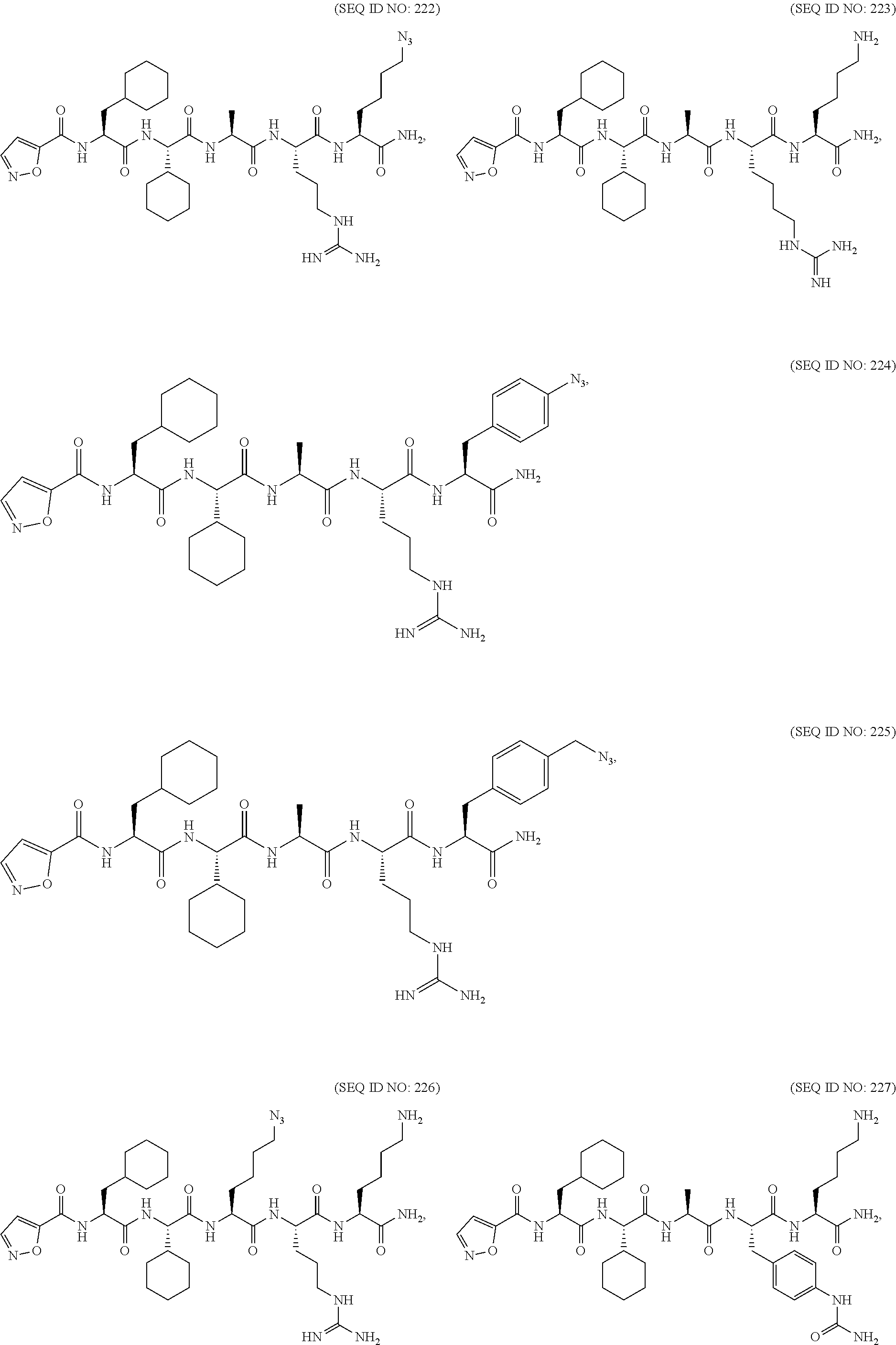

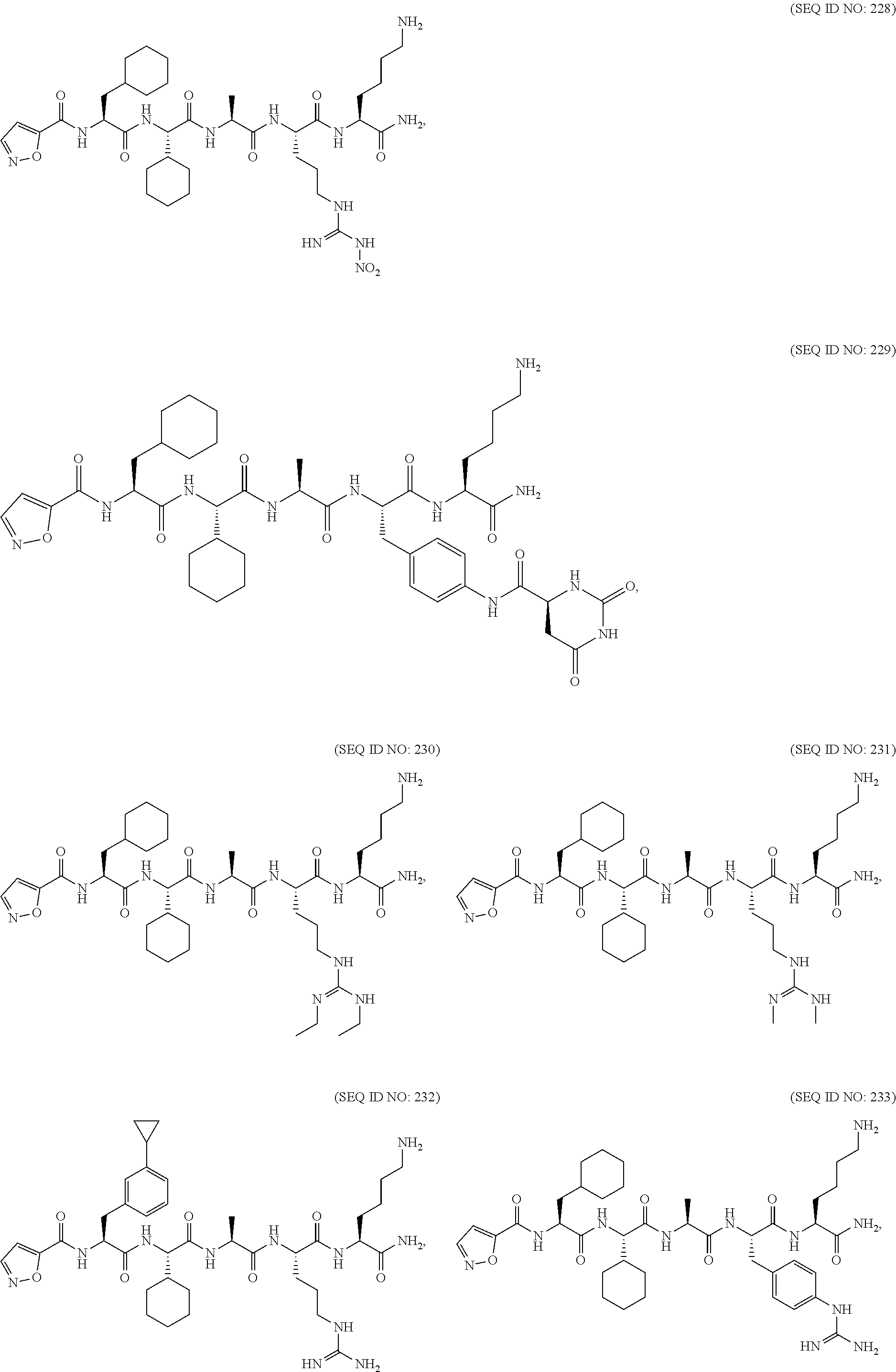

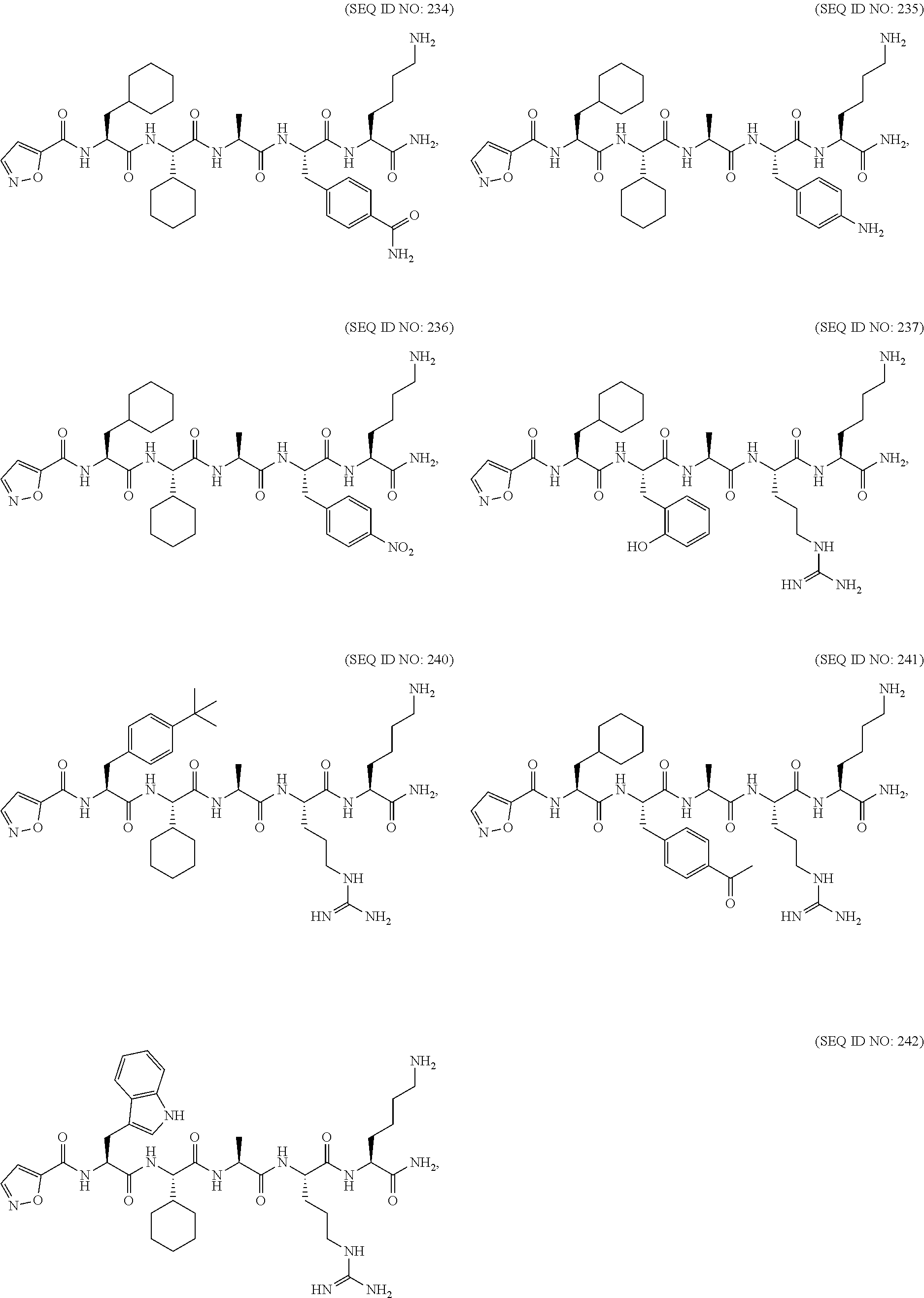

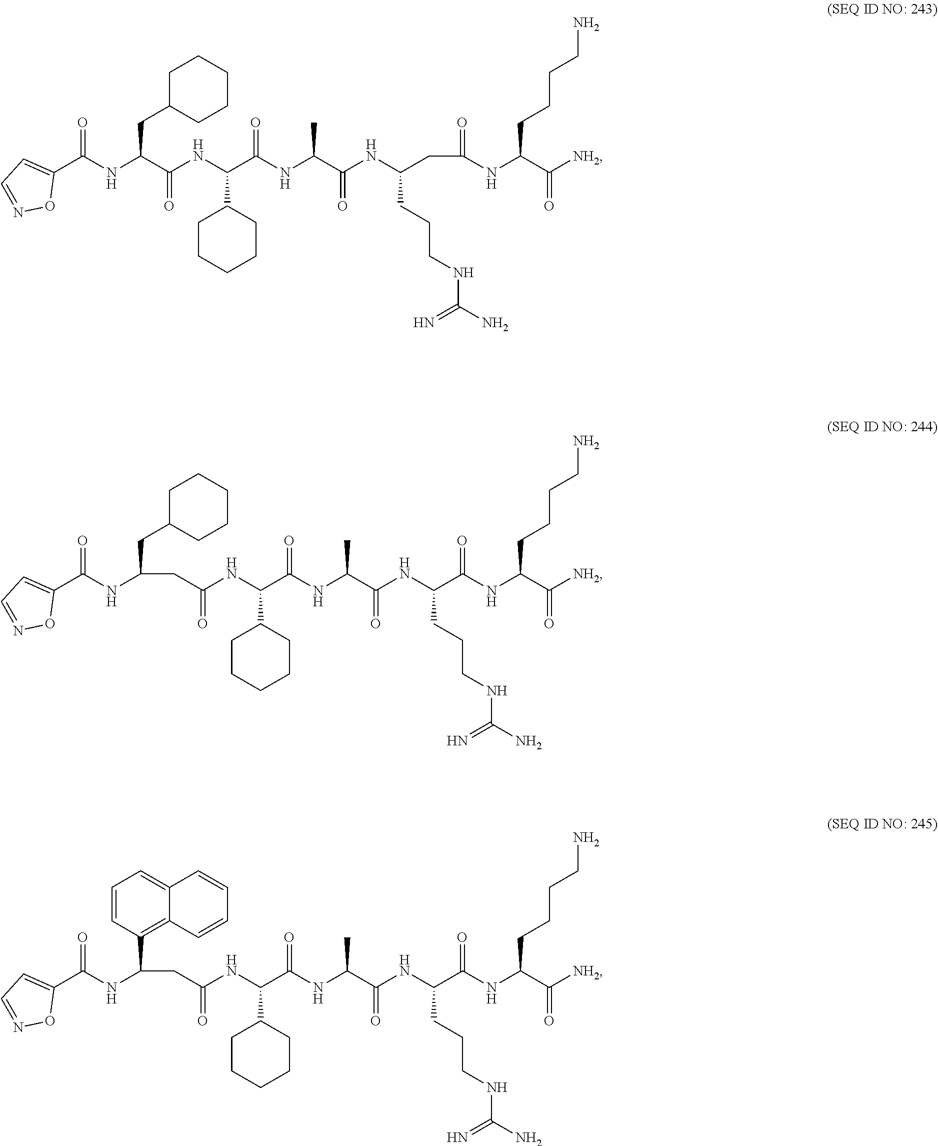 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)CC(=O)N[C@@H](CCCCN)C(N)=O ; C[C@H](NC(=O)[C@@H](NC(=O)C[C@H](CC1CCCCC1)NC(=O)c1ccno1)C1CCCCC1)C(=O)N[C@@H](CCCNC(=N)N)C(=O)N[C@@H](CCCCN)C(N)=O ; C[C@H](NC(=O)[C@@H](NC(=O)C[C@@H](NC(=O)c1ccno1)c1cccc2ccccc12)C1CCCCC1)C(=O)N[C@@H](CCCNC(=N)N)C(=O)N[C@@H](CCCCN)C(N)=O]

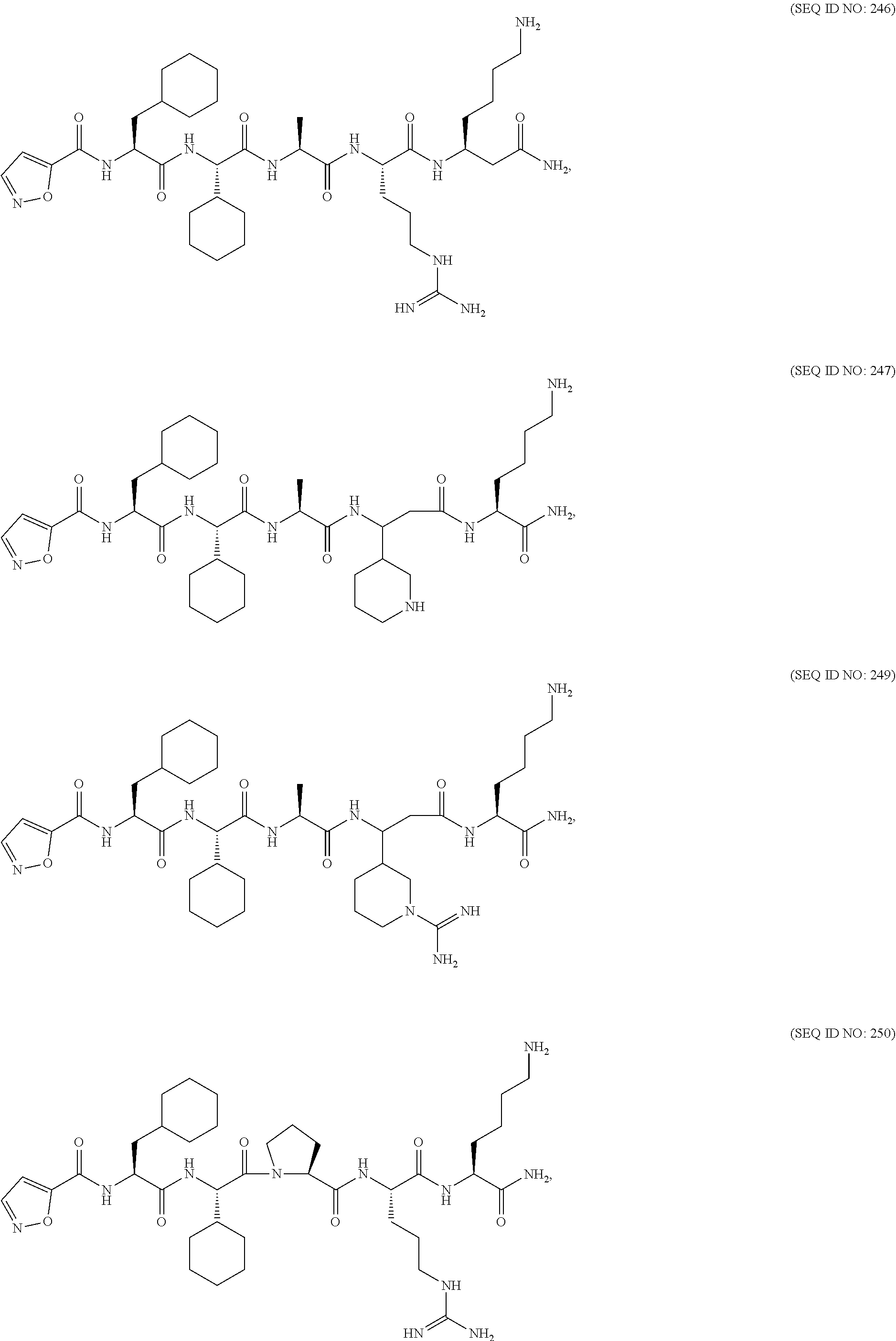 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCCN)CC(N)=O ; C[C@H](NC(=O)[C@@H](NC(=O)[C@H](CC1CCCCC1)NC(=O)c1ccno1)C1CCCCC1)C(=O)NC(CC(=O)N[C@@H](CCCCN)C(N)=O)C1CCCNC1 ; C[C@H](NC(=O)[C@@H](NC(=O)[C@H](CC1CCCCC1)NC(=O)c1ccno1)C1CCCCC1)C(=O)NC(CC(=O)N[C@@H](CCCCN)C(N)=O)C1CCCN(C(=N)N)C1 ; N=C(N)NCCC[C@H](NC(=O)[C@@H]1CCCN1C(=O)[C@@H](NC(=O)[C@H](CC1CCCCC1)NC(=O)c1ccno1)C1CCCCC1)C(=O)N[C@@H](CCCCN)C(N)=O]

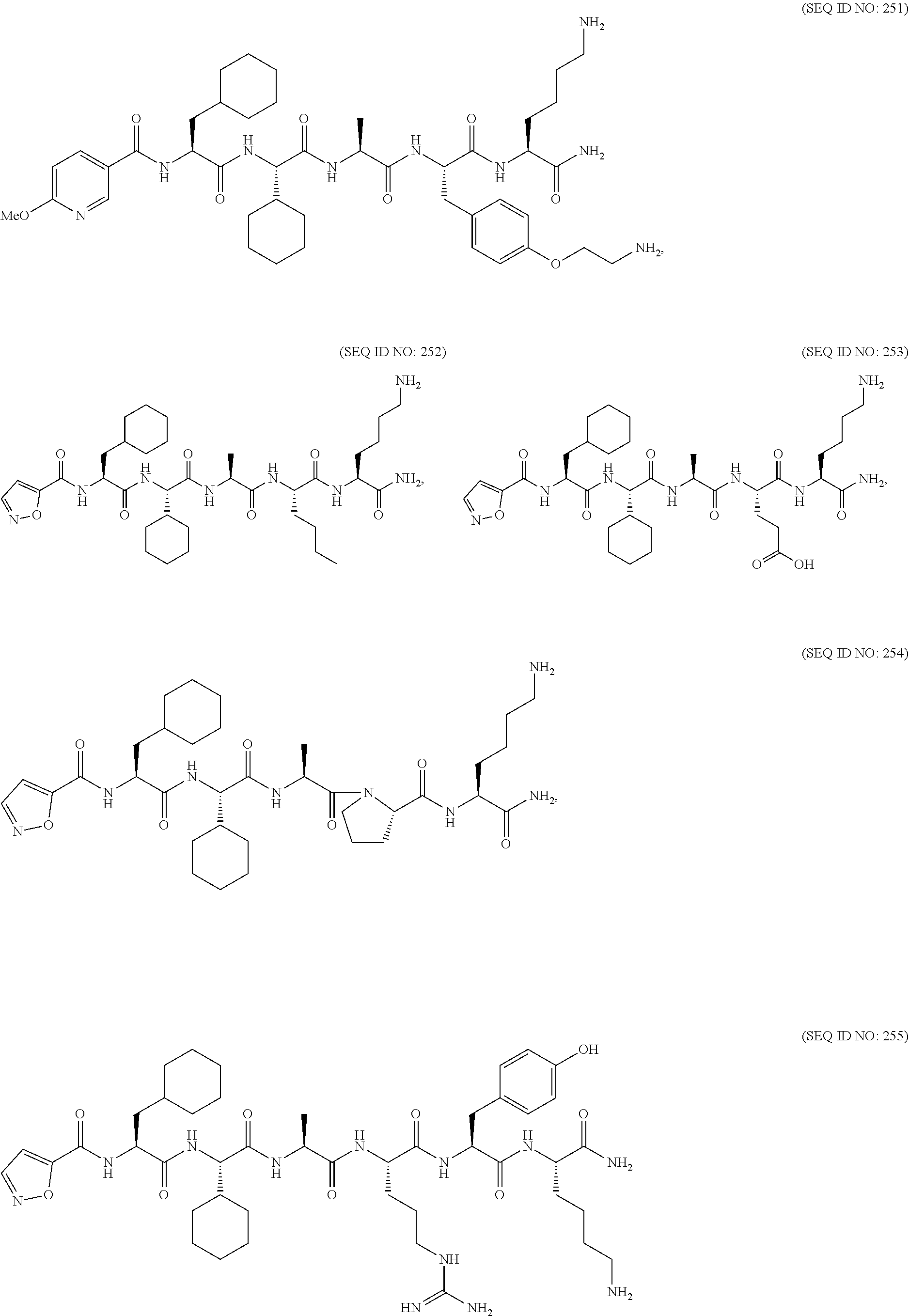

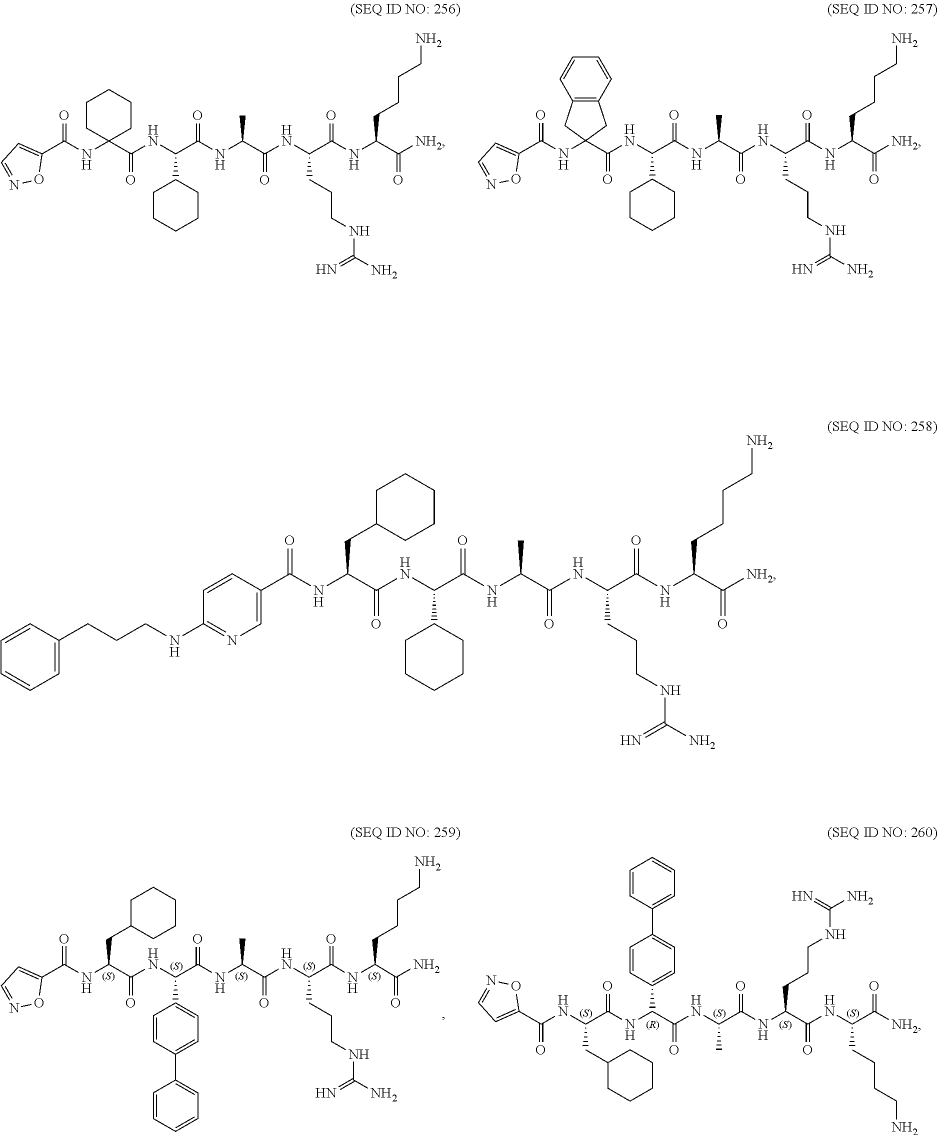

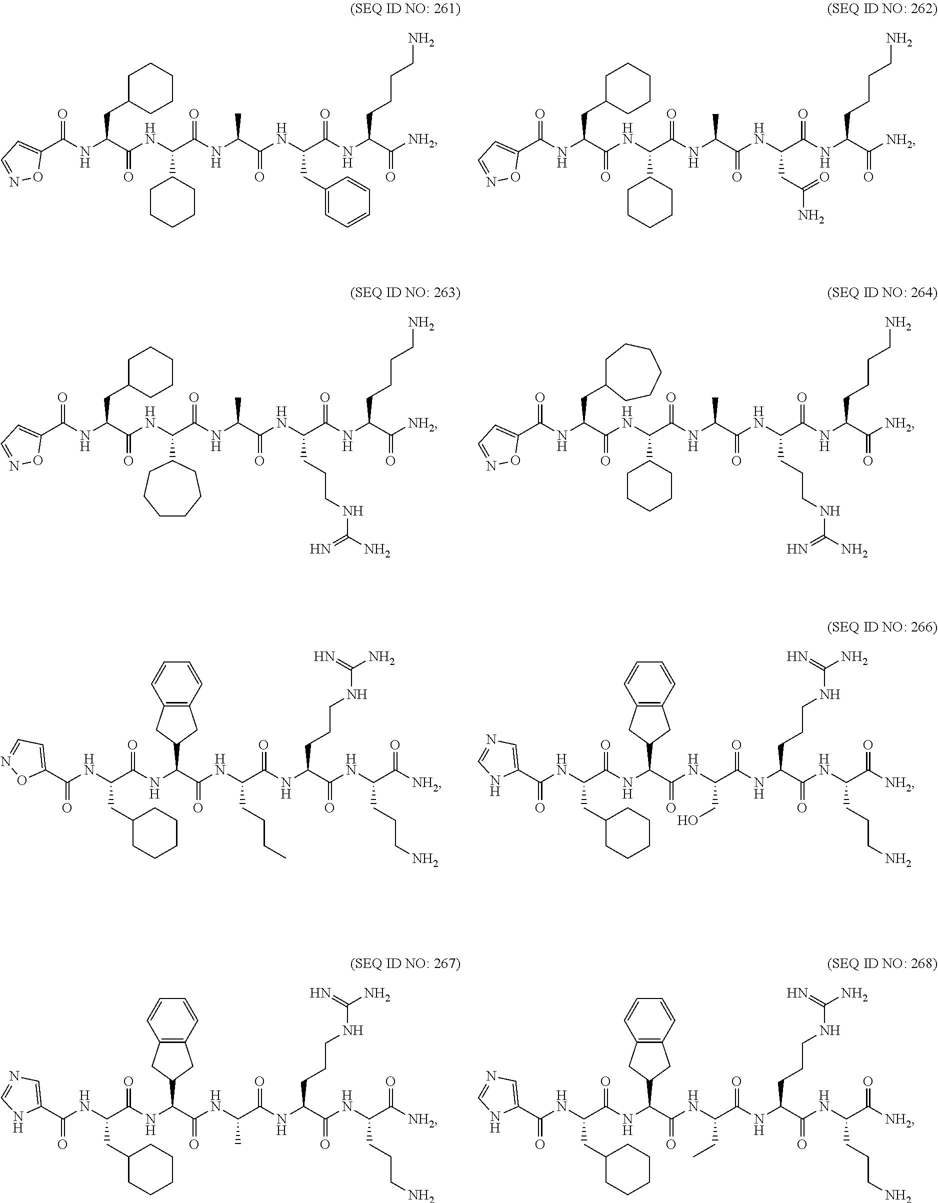

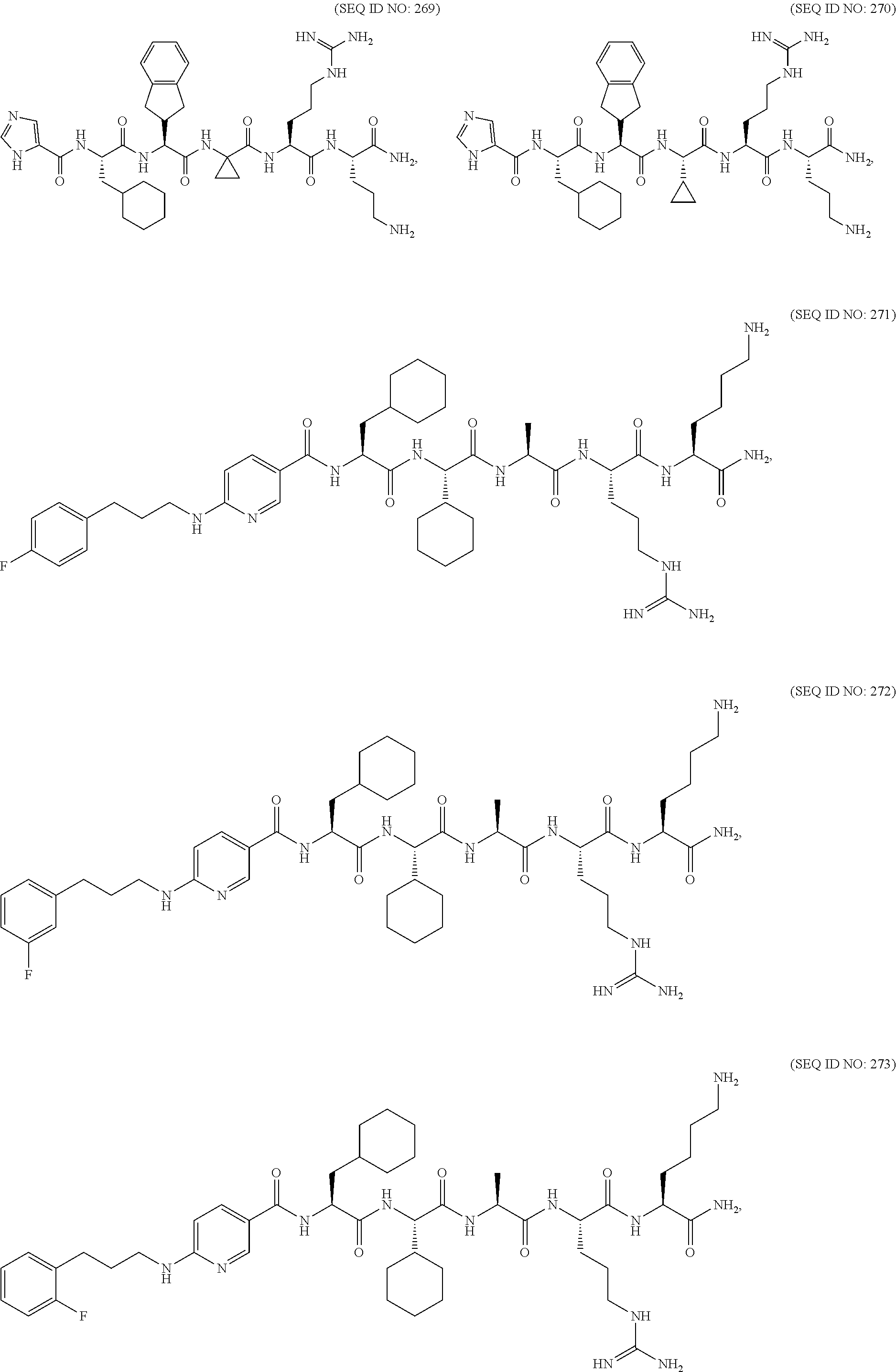

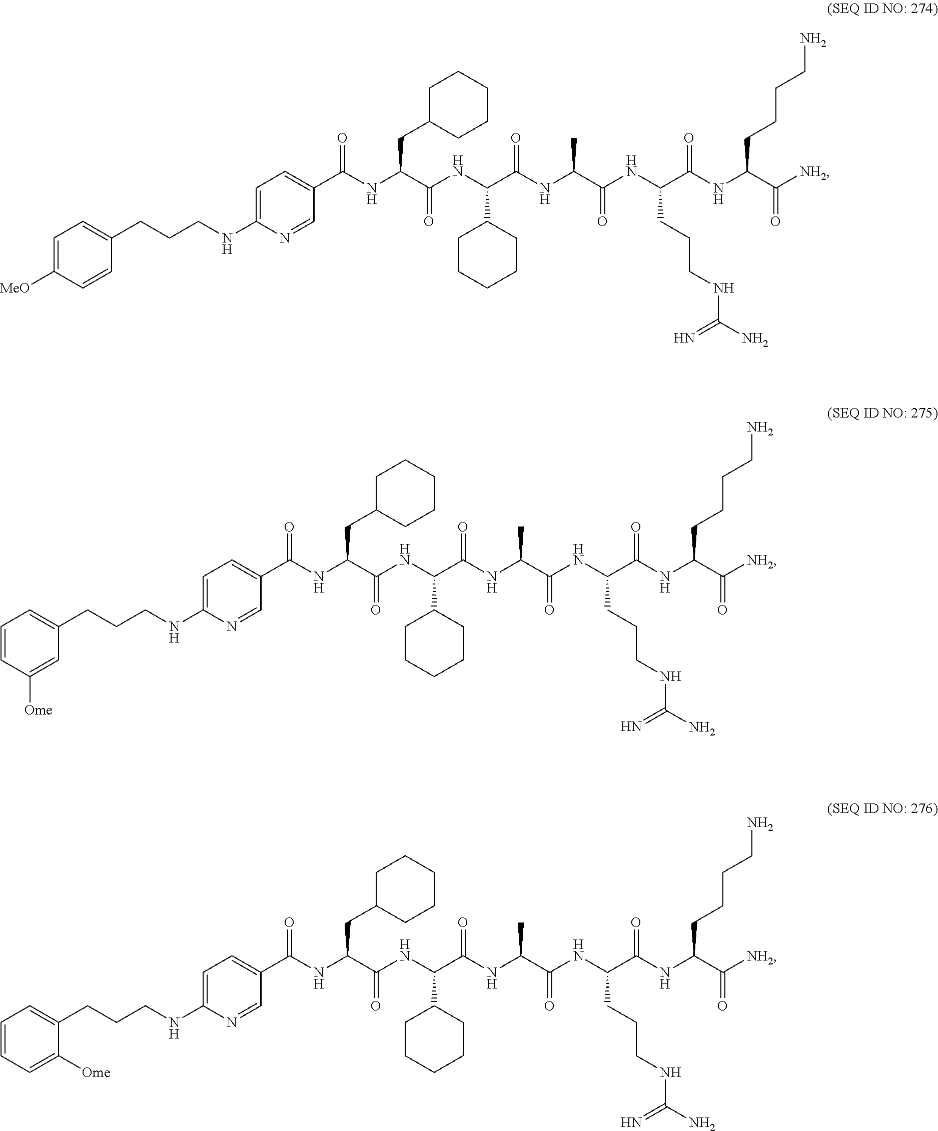

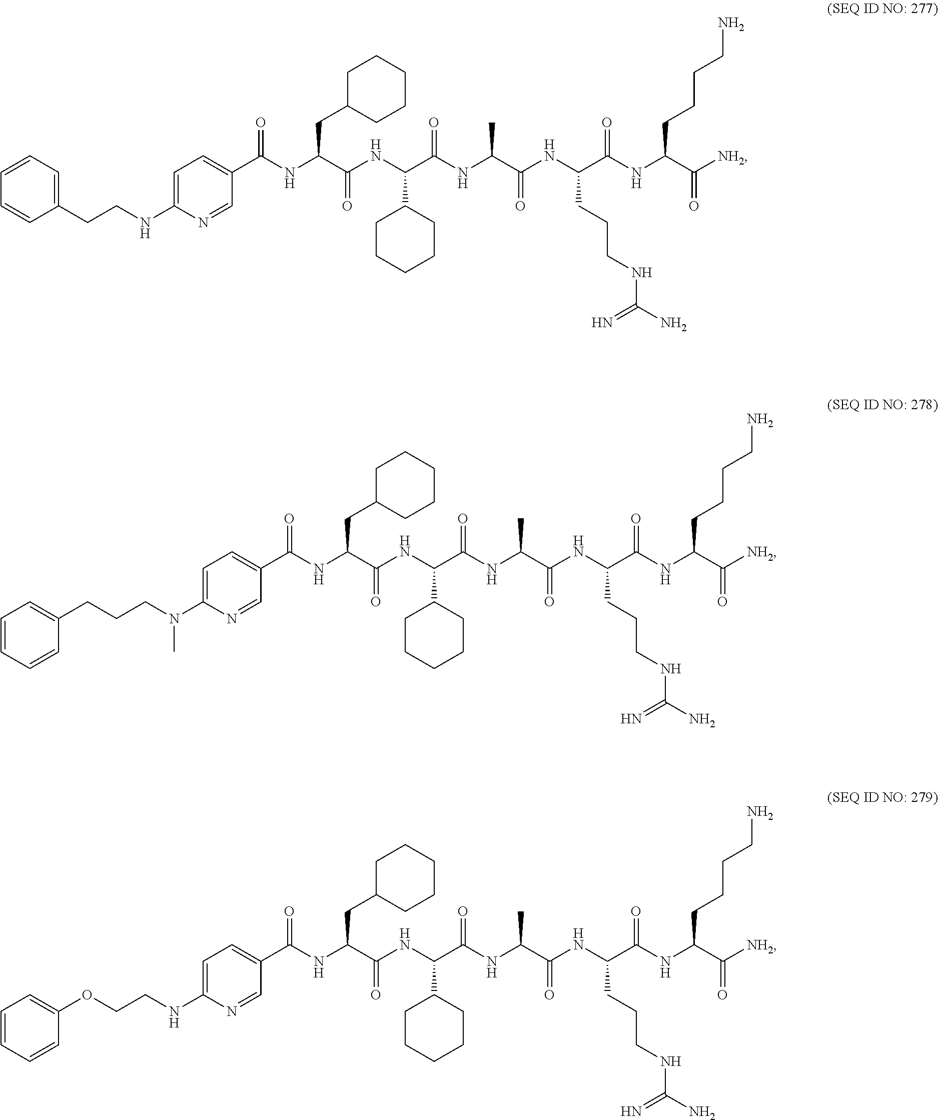 [SMILES: C[C@H](NC(=O)[C@@H](NC(=O)[C@H](CC1CCCCC1)NC(=O)c1ccc(NCCc2ccccc2)nc1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccc(N(C)CCCc2ccccc2)nc1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccc(NCCOc2ccccc2)nc1)C1CCCCC1)C(=O)N[C@@H](CCCNC(=N)N)C(=O)N[C@@H](CCCCN)C(N)=O]

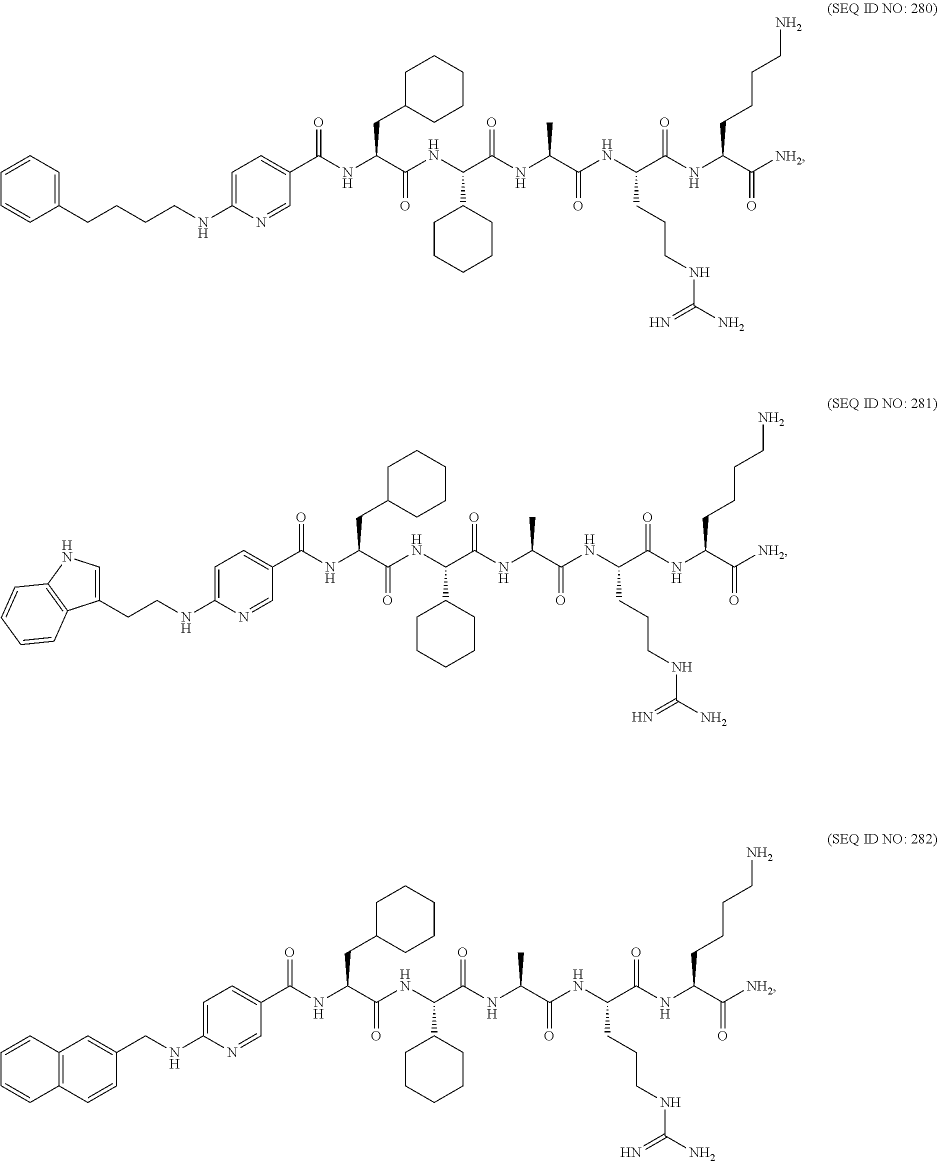 [SMILES: C[C@H](NC(=O)[C@@H](NC(=O)[C@H](CC1CCCCC1)NC(=O)c1ccc(NCCCCc2ccccc2)nc1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccc(NCCc2c[nH]c3ccccc23)nc1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccc(NCc2ccc3ccccc3c2)nc1)C1CCCCC1)C(=O)N[C@@H](CCCNC(=N)N)C(=O)N[C@@H](CCCCN)C(N)=O]

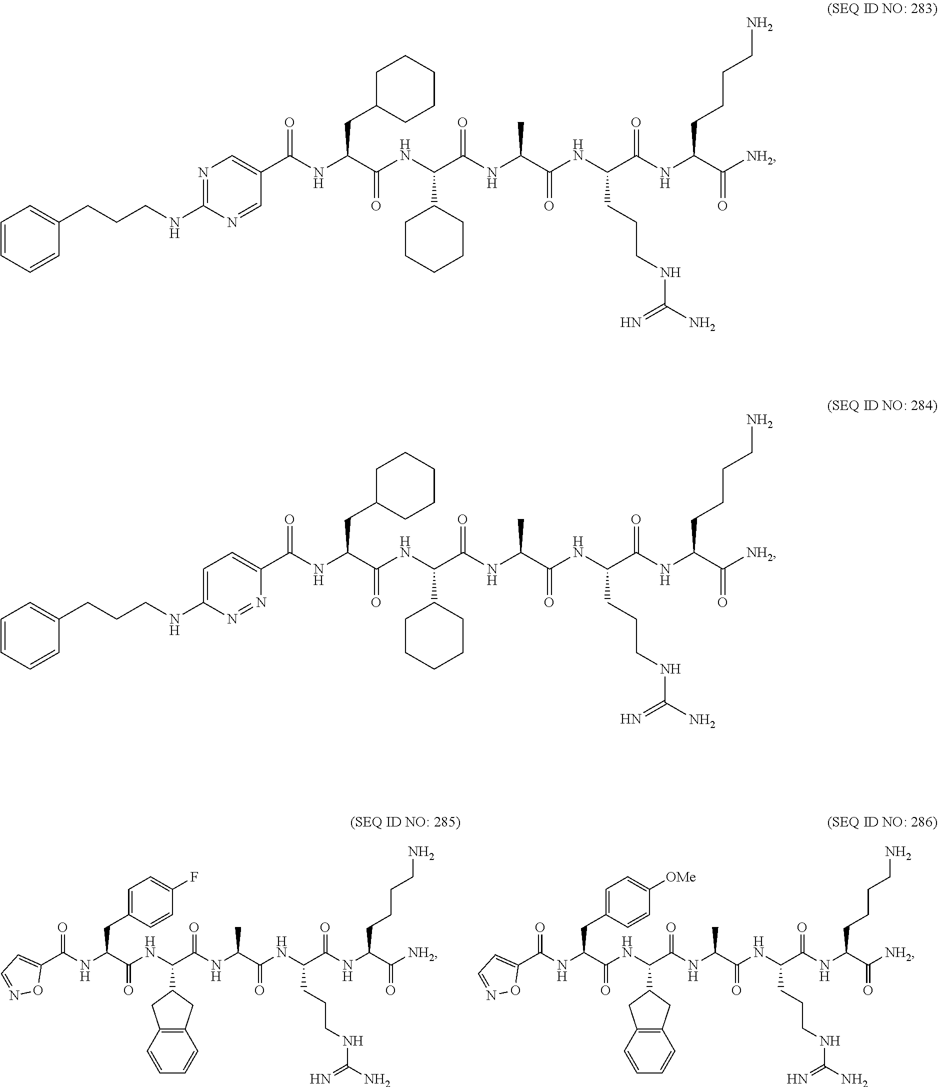

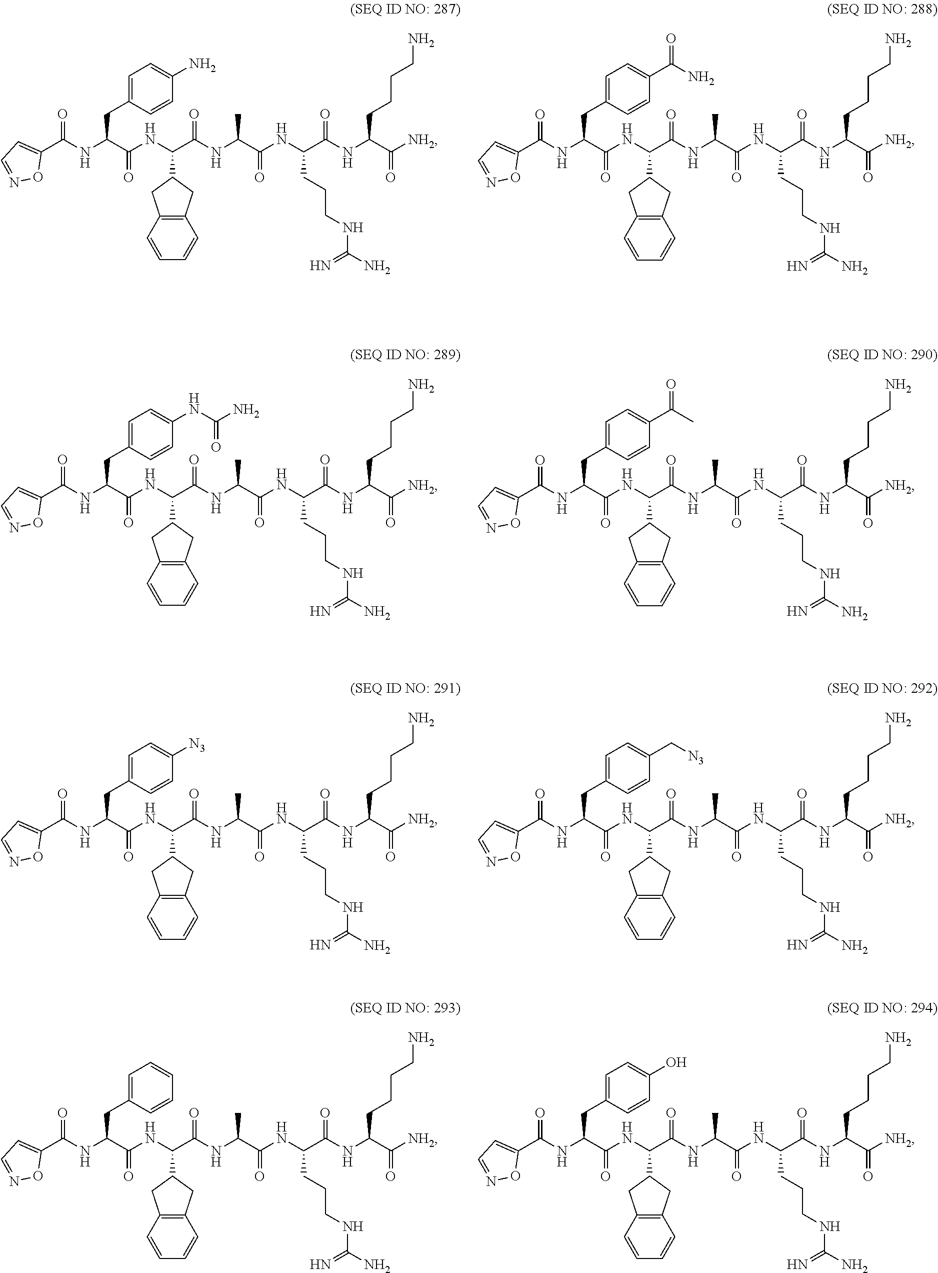

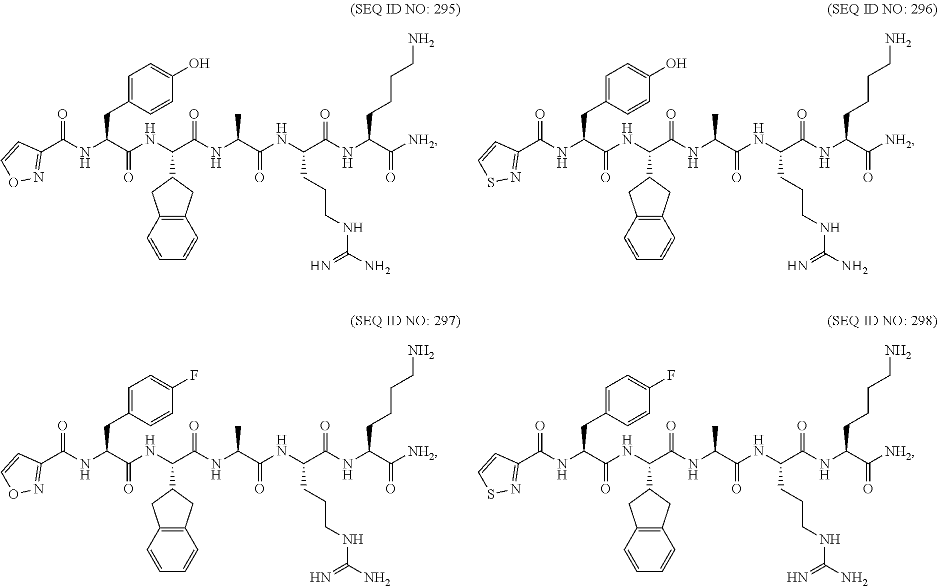 [SMILES: C[C@H](NC(=O)[C@@H](NC(=O)[C@H](Cc1ccc(O)cc1)NC(=O)c1ccon1)C1Cc2ccccc2C1)C(=O)N[C@@H](CCCNC(=N)N)C(=O)N[C@@H](CCCCN)C(N)=O ; C[C@H](NC(=O)[C@@H](NC(=O)[C@H](Cc1ccc(O)cc1)NC(=O)c1ccsn1)C1Cc2ccccc2C1)C(=O)N[C@@H](CCCNC(=N)N)C(=O)N[C@@H](CCCCN)C(N)=O ; C[C@H](NC(=O)[C@@H](NC(=O)[C@H](Cc1ccc(F)cc1)NC(=O)c1ccon1)C1Cc2ccccc2C1)C(=O)N[C@@H](CCCNC(=N)N)C(=O)N[C@@H](CCCCN)C(N)=O ; C[C@H](NC(=O)[C@@H](NC(=O)[C@H](Cc1ccc(F)cc1)NC(=O)c1ccsn1)C1Cc2ccccc2C1)C(=O)N[C@@H](CCCNC(=N)N)C(=O)N[C@@H](CCCCN)C(N)=O]

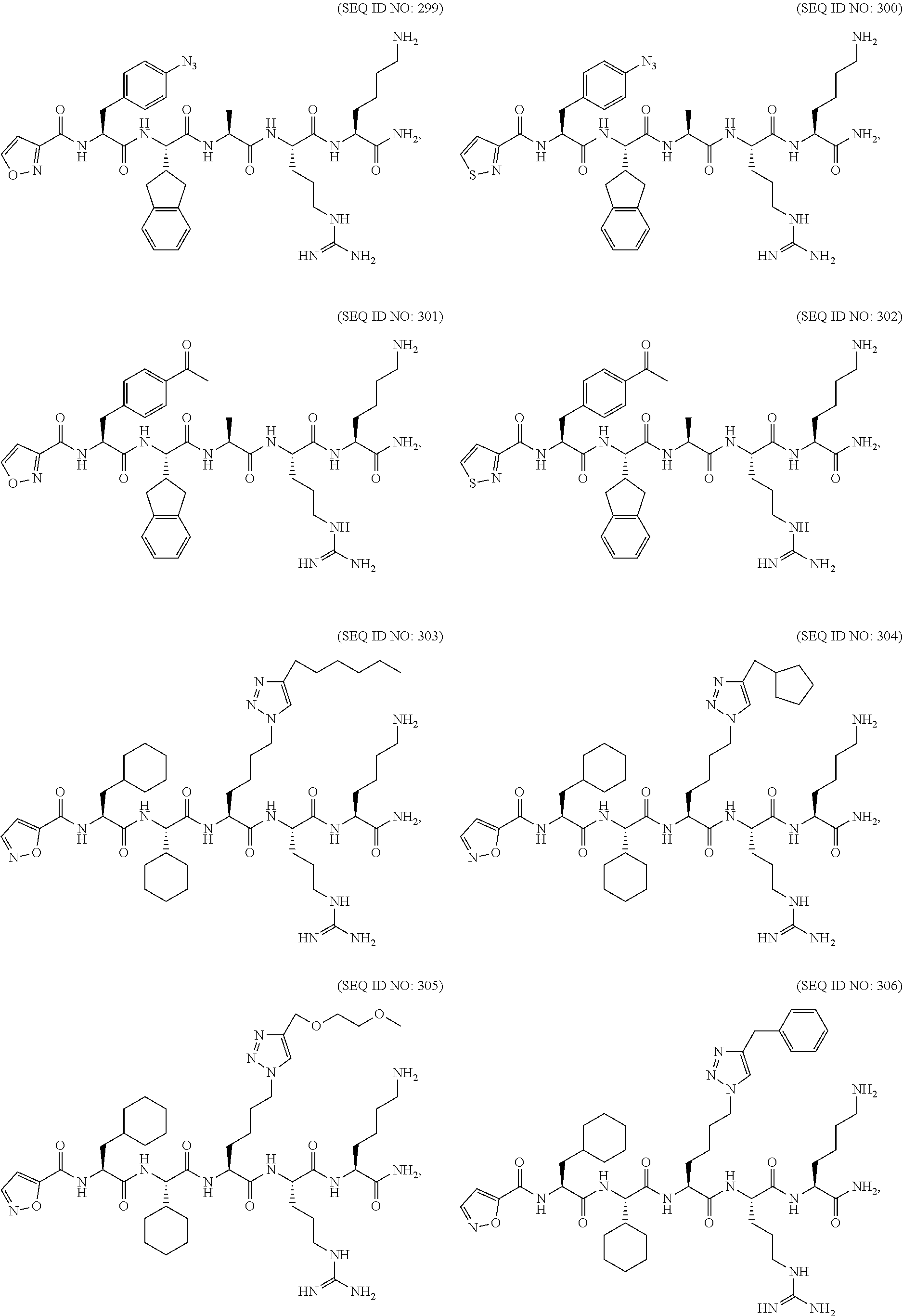

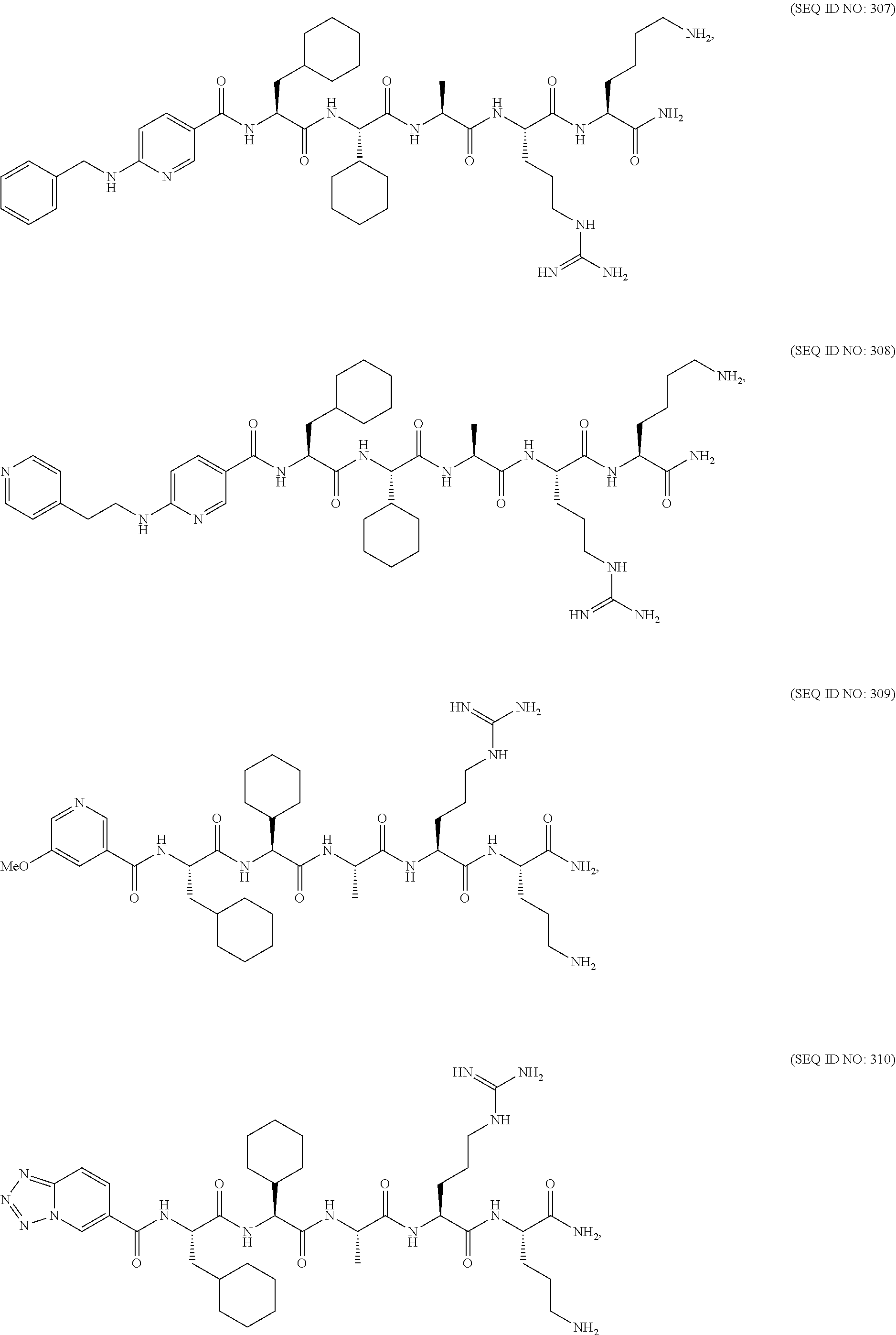

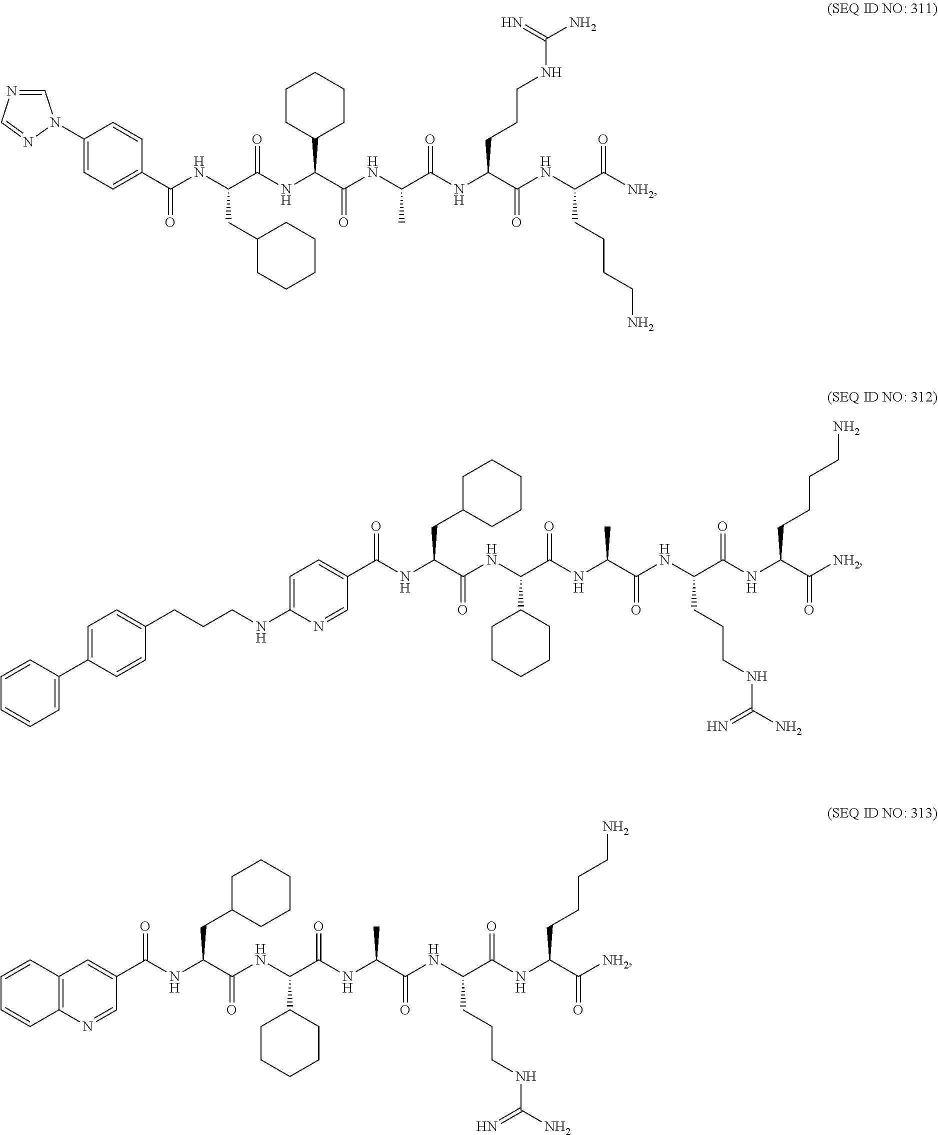 [SMILES: C[C@H](NC(=O)[C@@H](NC(=O)[C@H](CC1CCCCC1)NC(=O)c1ccc(-n2cncn2)cc1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccc(NCCCc2ccc(-c3ccccc3)cc2)nc1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1cnc2ccccc2c1)C1CCCCC1)C(=O)N[C@@H](CCCNC(=N)N)C(=O)N[C@@H](CCCCN)C(N)=O]

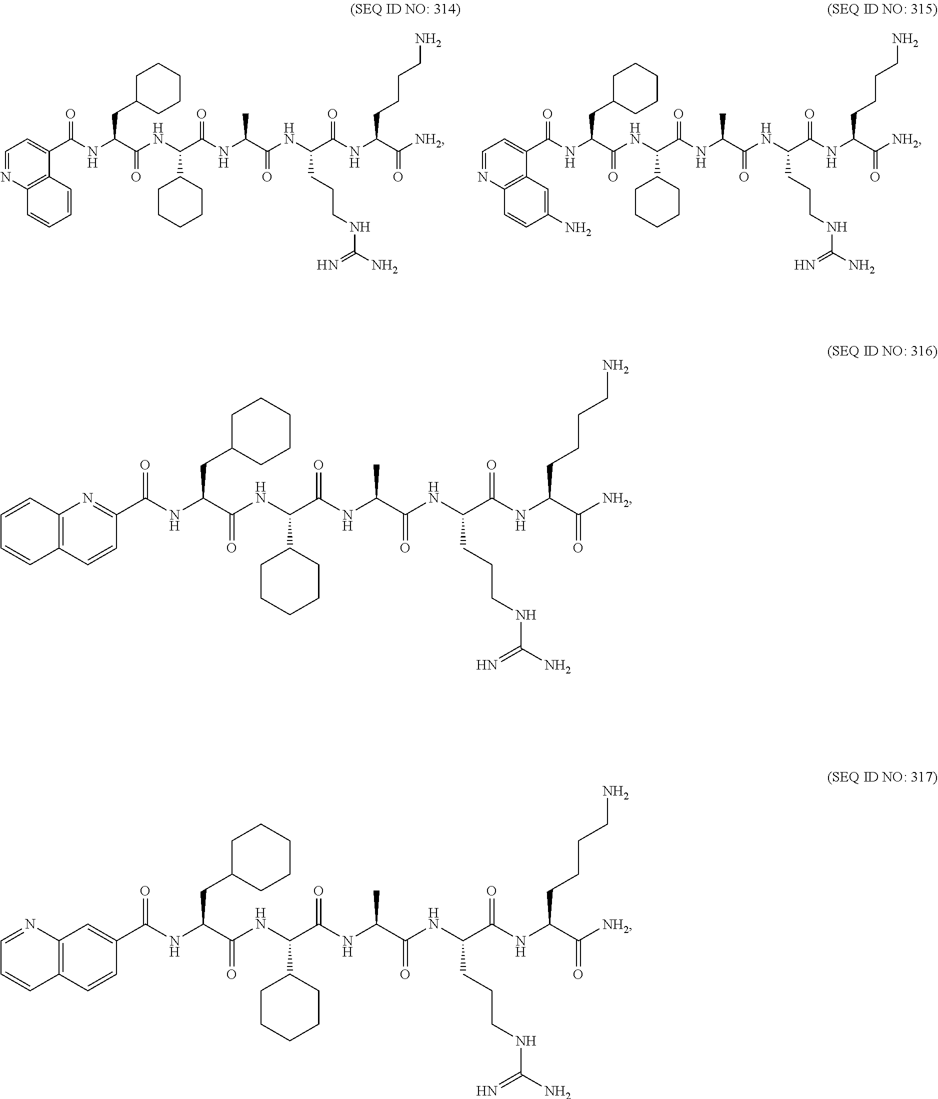 [SMILES: C[C@H](NC(=O)[C@@H](NC(=O)[C@H](CC1CCCCC1)NC(=O)c1ccnc2ccccc12)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccnc2ccc(N)cc12)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccc2ccccc2n1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccc2cccnc2c1)C1CCCCC1)C(=O)N[C@@H](CCCNC(=N)N)C(=O)N[C@@H](CCCCN)C(N)=O]

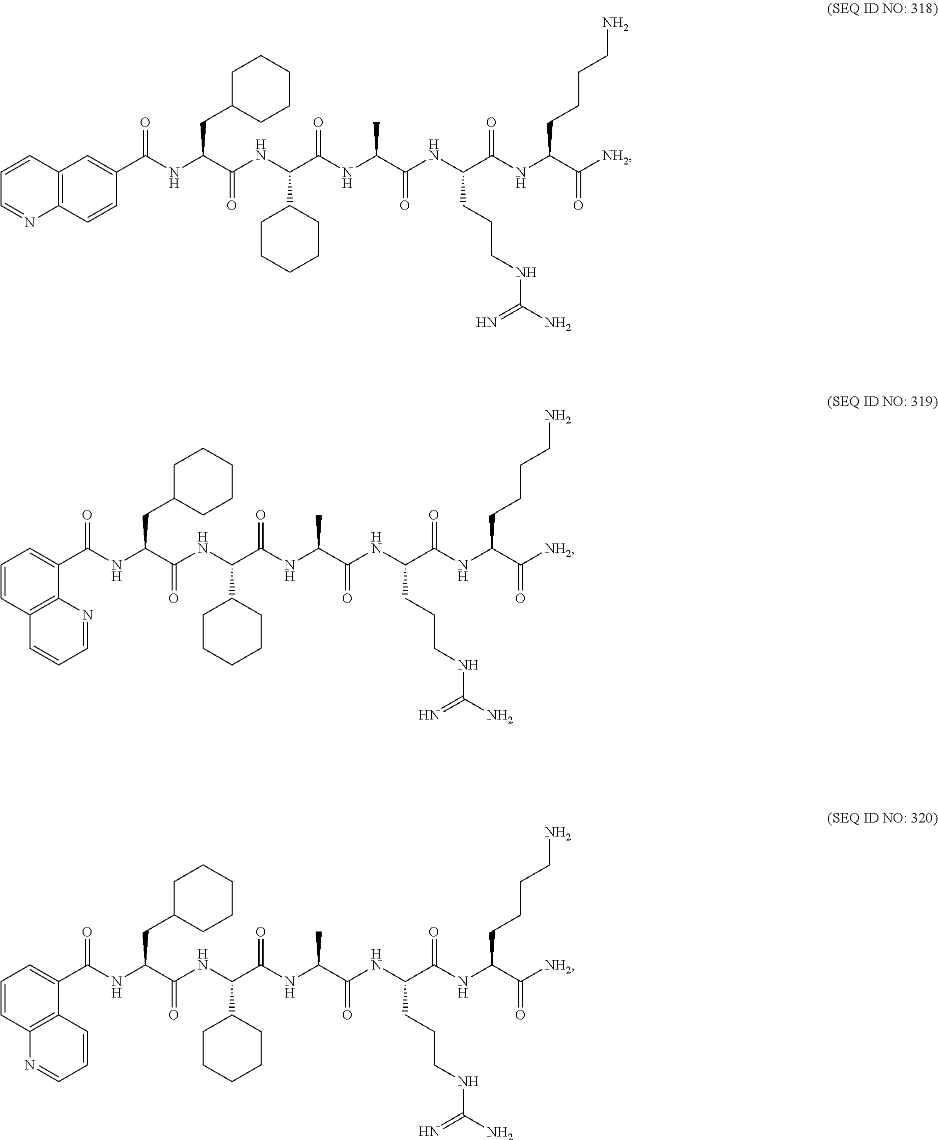 [SMILES: C[C@H](NC(=O)[C@@H](NC(=O)[C@H](CC1CCCCC1)NC(=O)c1ccc2ncccc2c1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1cccc2cccnc12)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1cccc2ncccc12)C1CCCCC1)C(=O)N[C@@H](CCCNC(=N)N)C(=O)N[C@@H](CCCCN)C(N)=O]

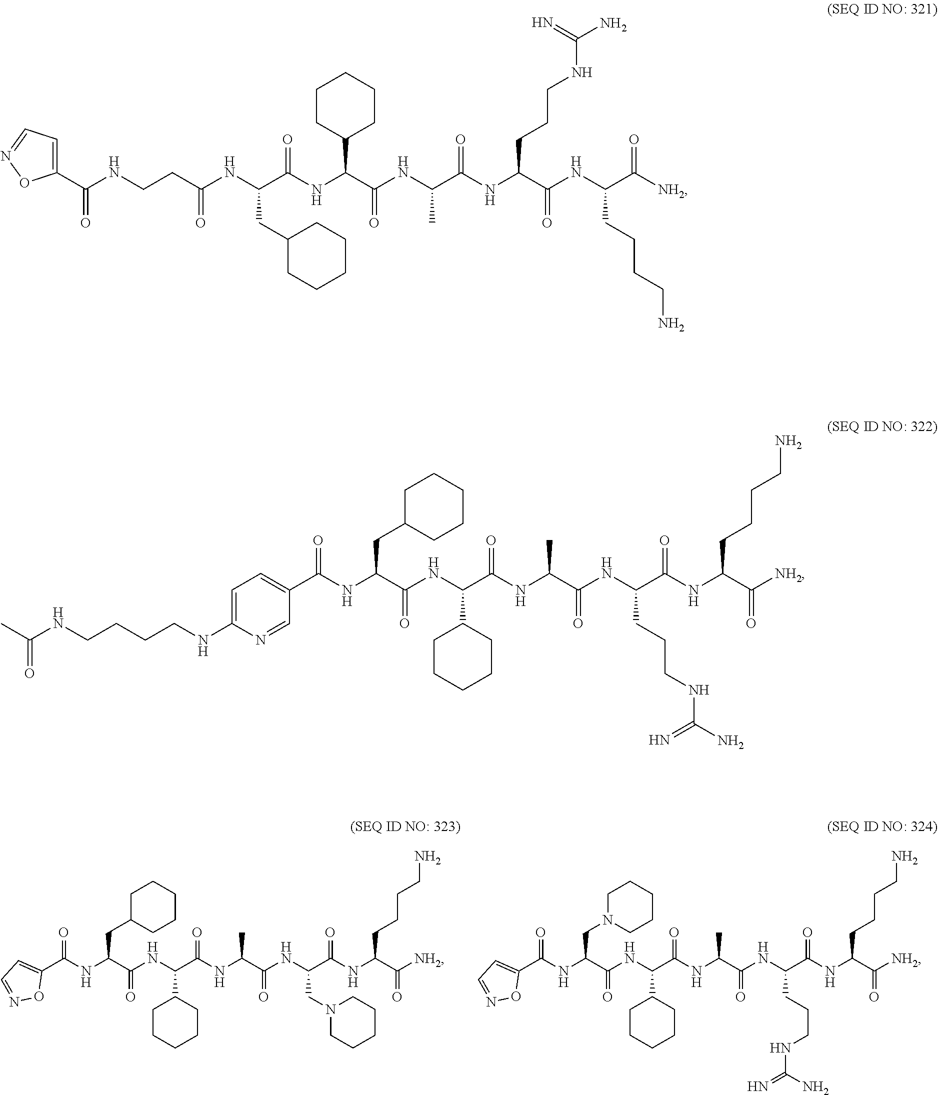 [SMILES: C[C@H](NC(=O)[C@@H](NC(=O)[C@H](CC1CCCCC1)NC(=O)CCNC(=O)c1ccno1)C1CCCCC1)C(=O)N[C@@H](CCCNC(=N)N)C(=O)N[C@@H](CCCCN)C(N)=O ; CC(=O)NCCCCNc1ccc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)cn1 ; C[C@H](NC(=O)[C@@H](NC(=O)[C@H](CC1CCCCC1)NC(=O)c1ccno1)C1CCCCC1)C(=O)N[C@@H](CN1CCCCC1)C(=O)N[C@@H](CCCCN)C(N)=O ; C[C@H](NC(=O)[C@@H](NC(=O)[C@H](CN1CCCCC1)NC(=O)c1ccno1)C1CCCCC1)C(=O)N[C@@H](CCCNC(=N)N)C(=O)N[C@@H](CCCCN)C(N)=O]

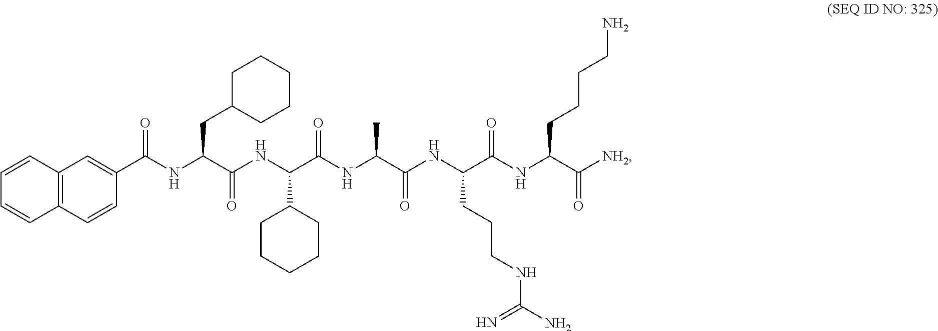 [SMILES: C[C@H](NC(=O)[C@@H](NC(=O)[C@H](CC1CCCCC1)NC(=O)c1ccc2ccccc2c1)C1CCCCC1)C(=O)N[C@@H](CCCNC(=N)N)C(=O)N[C@@H](CCCCN)C(N)=O]

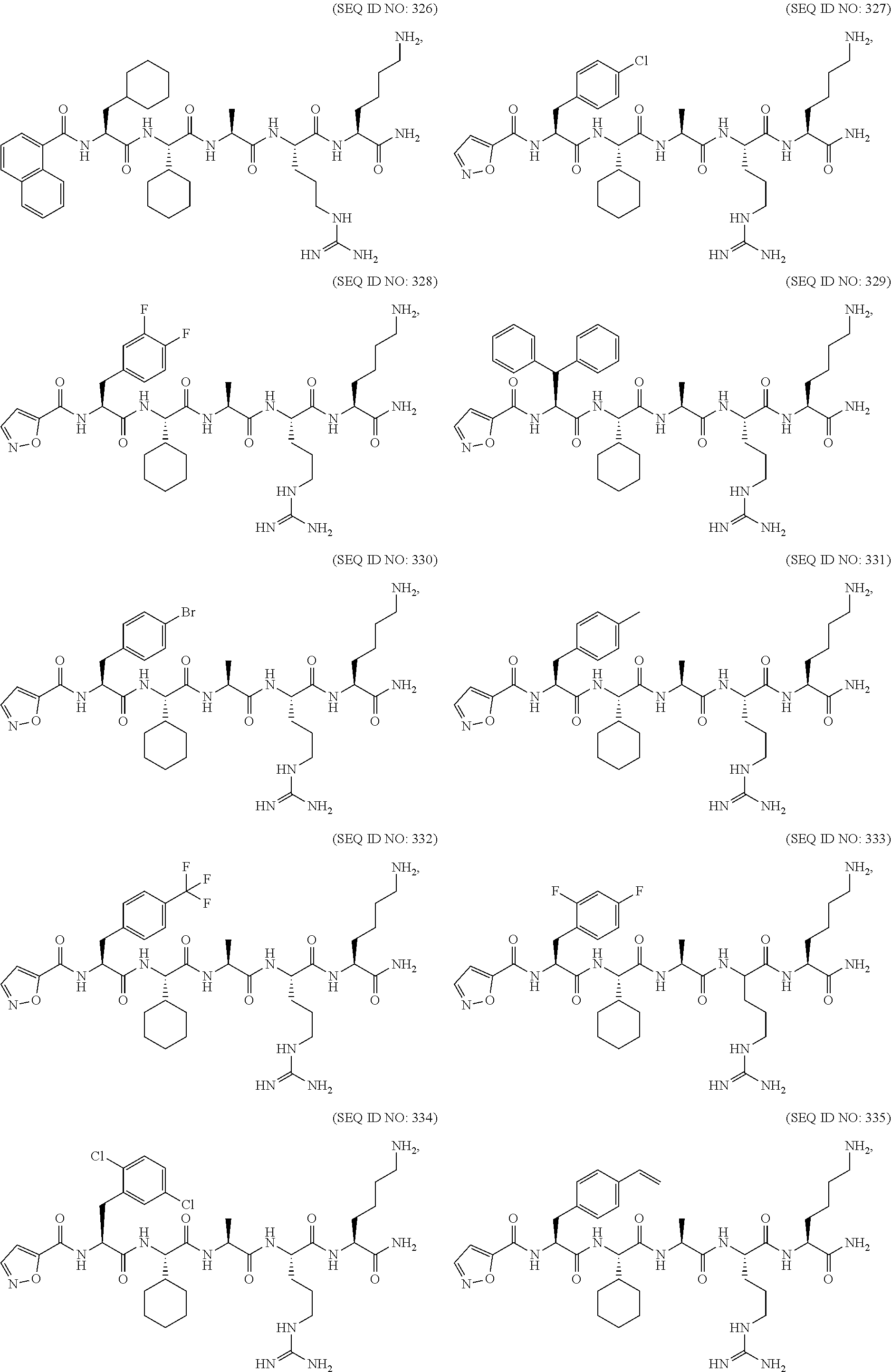

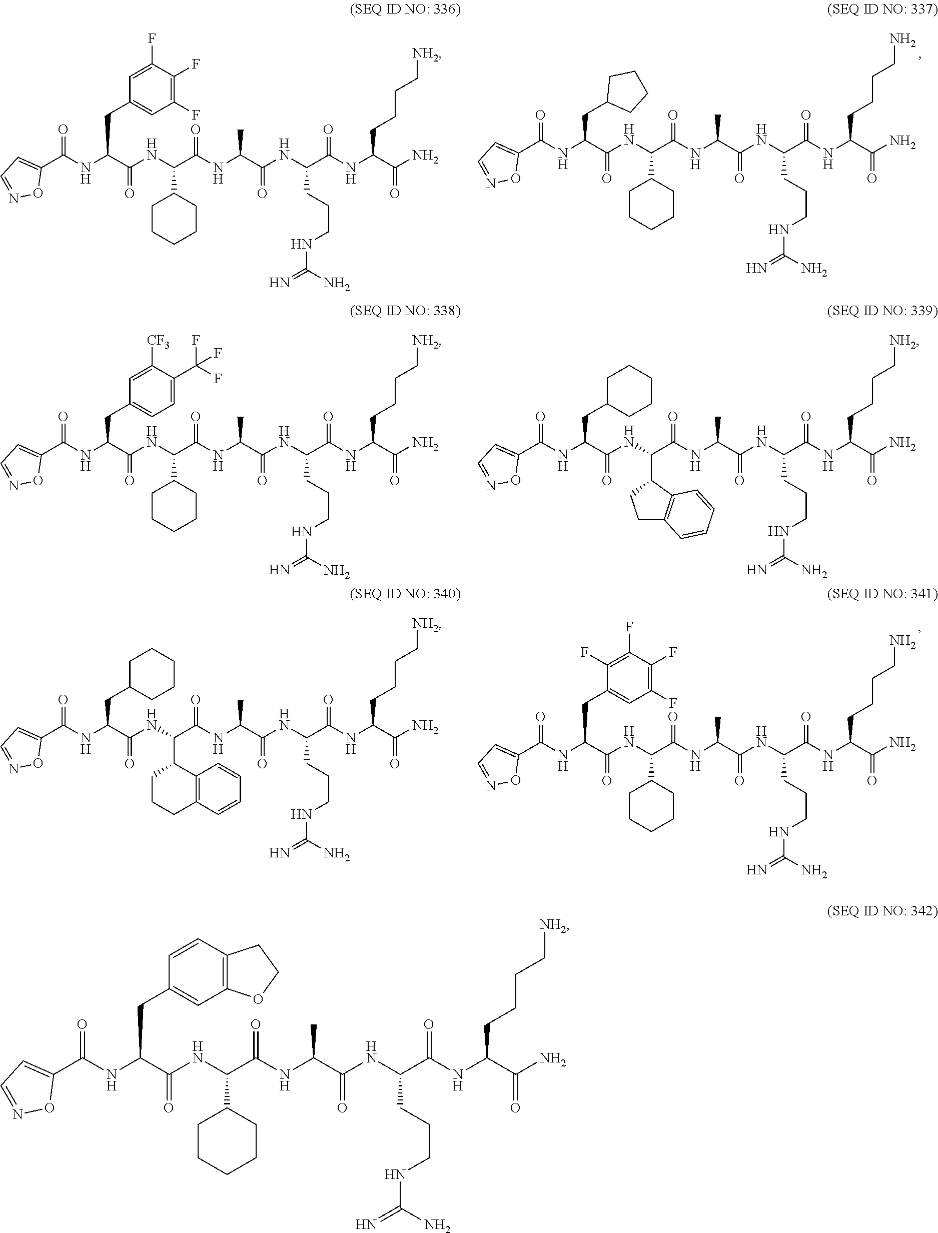

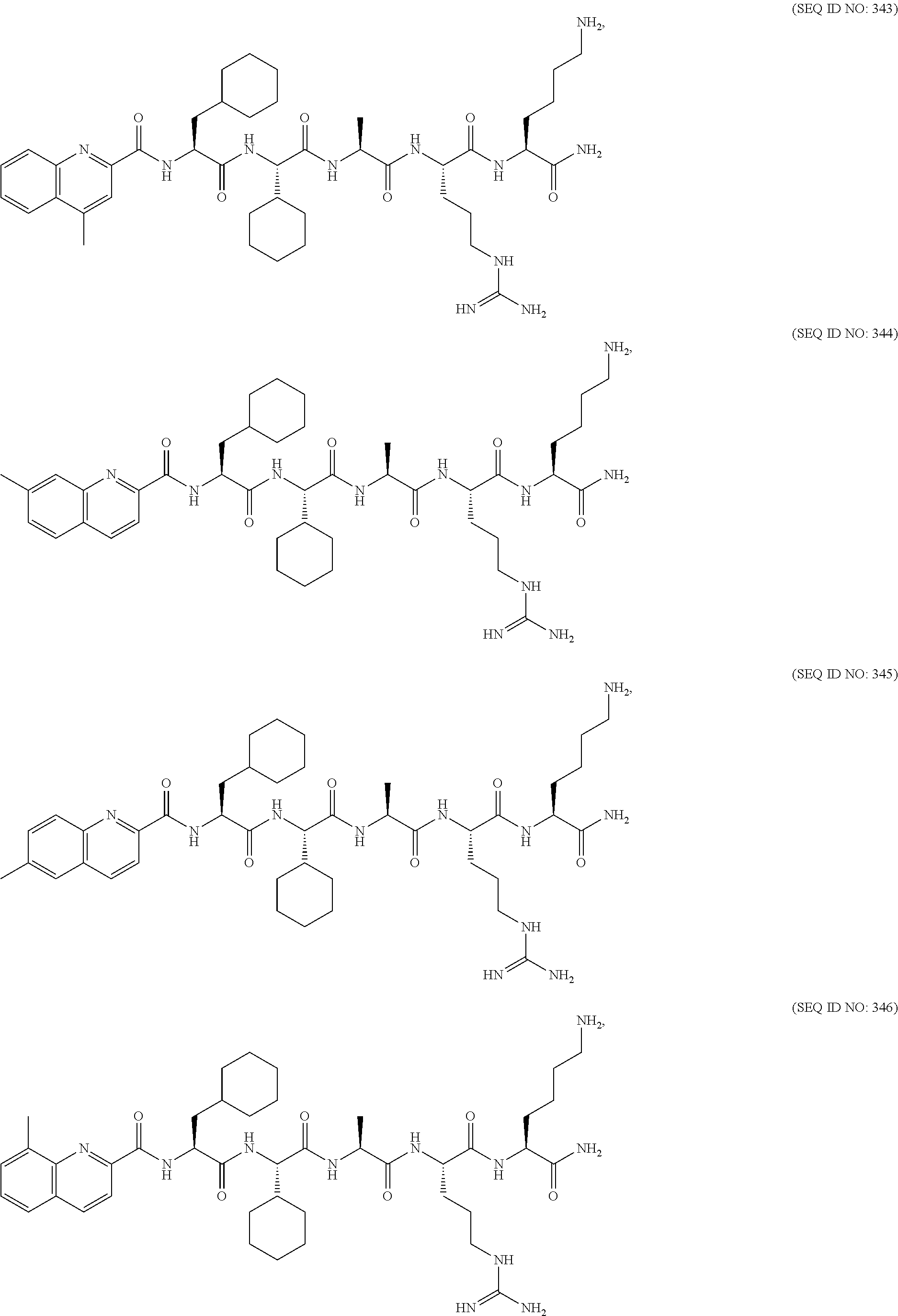

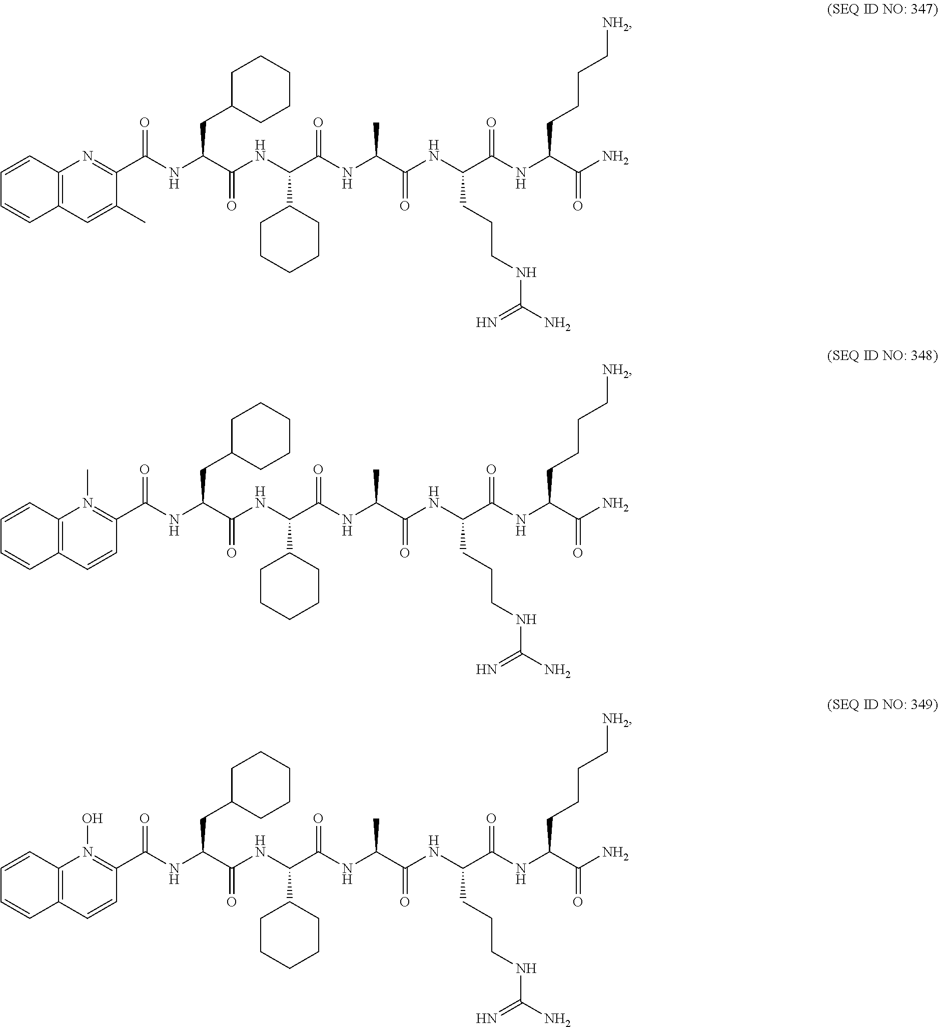

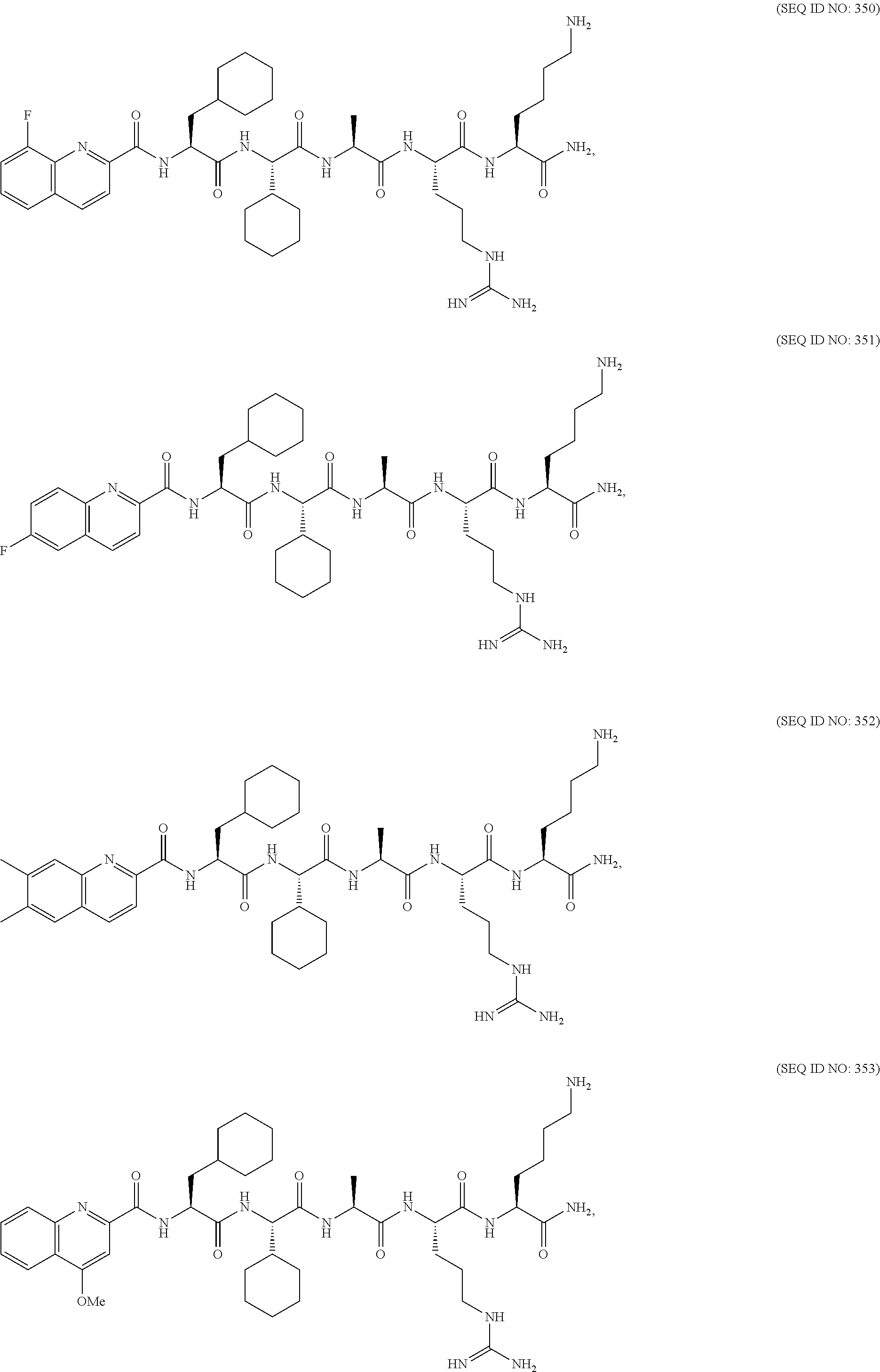

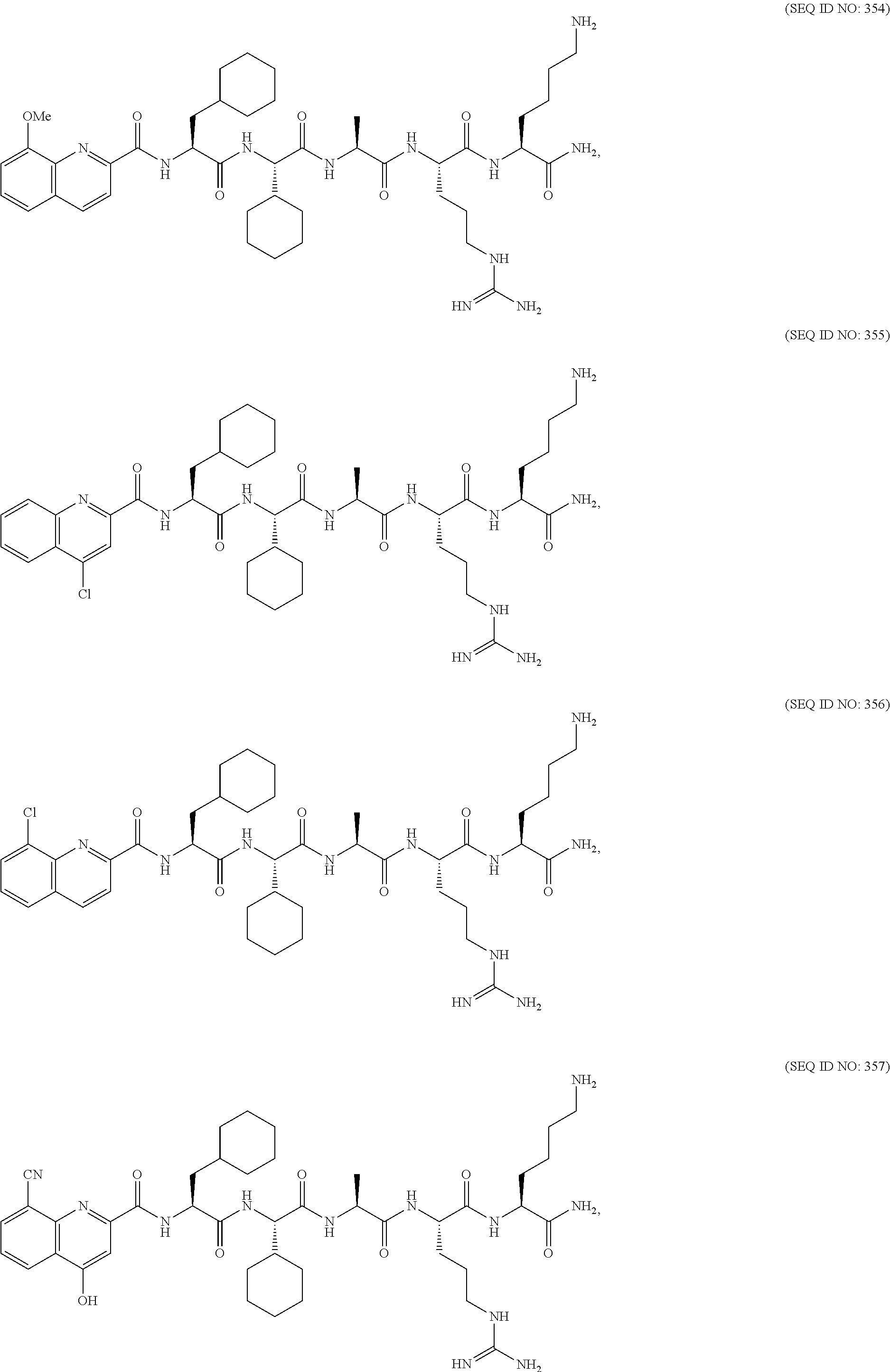

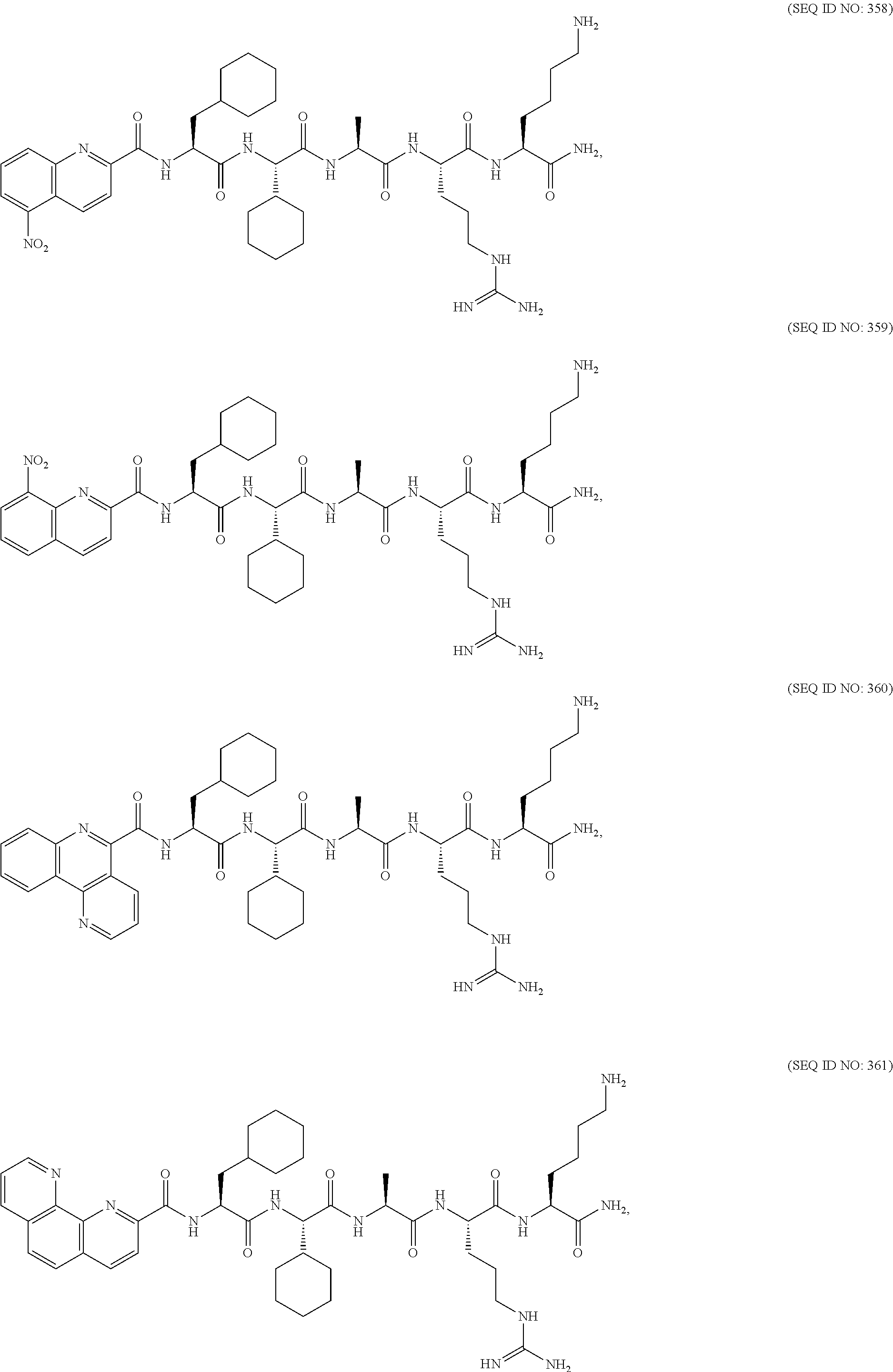

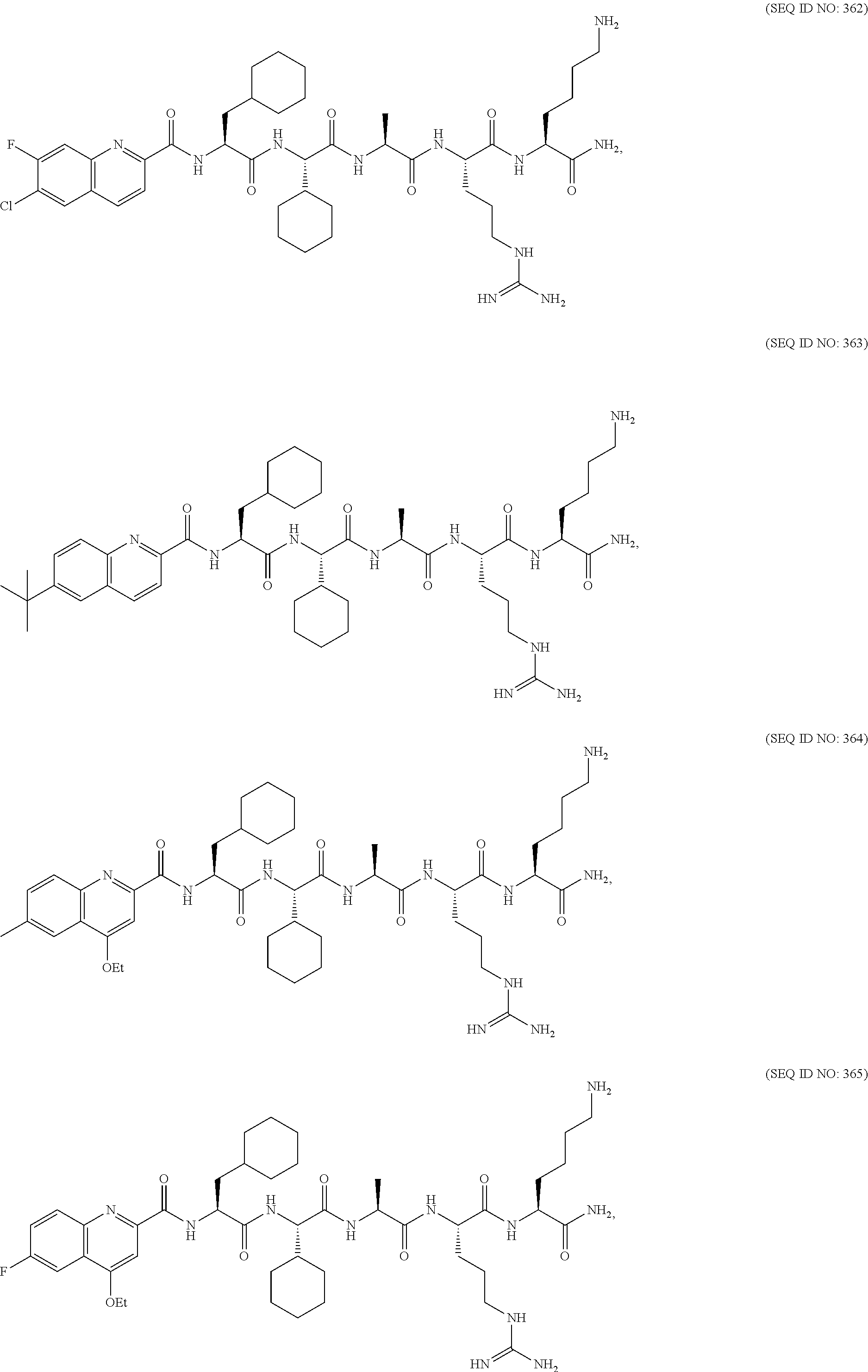

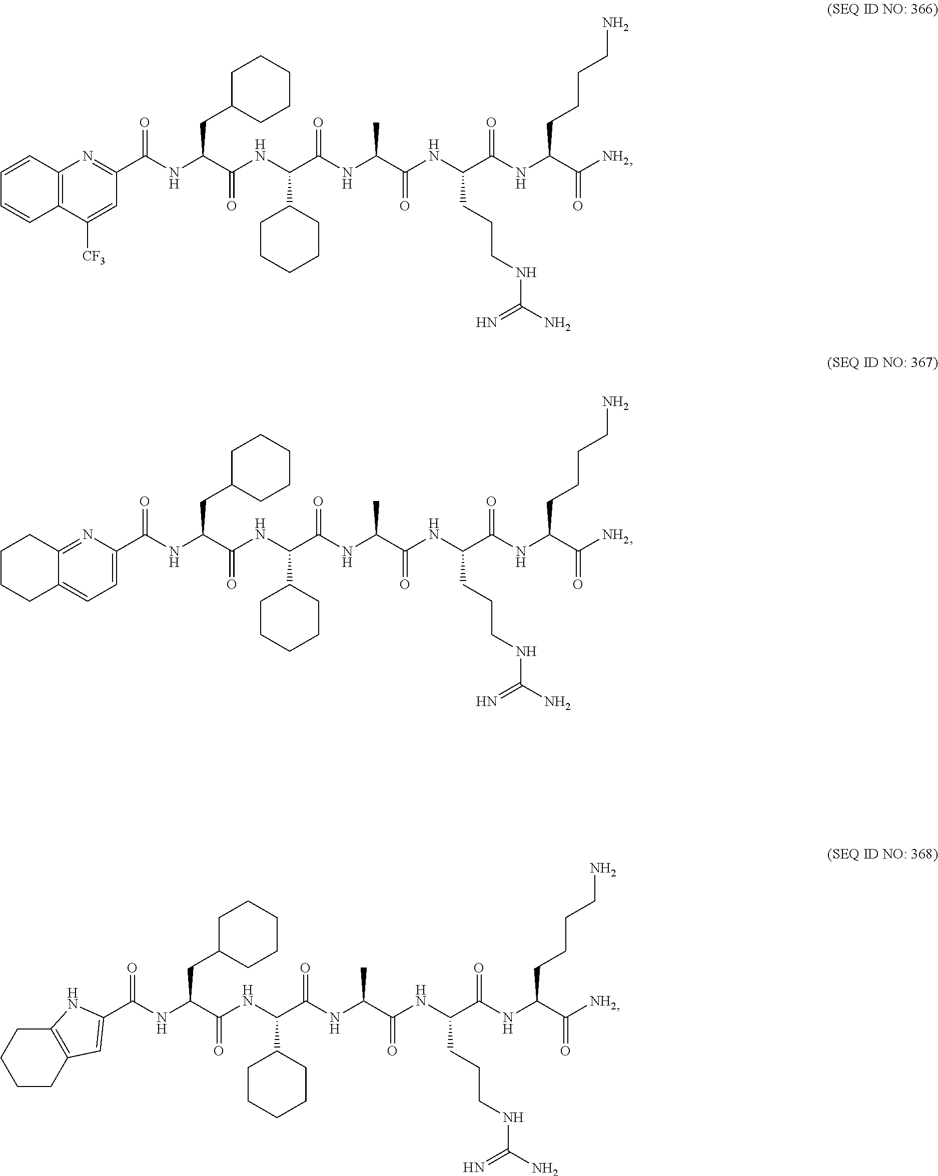 [SMILES: C[C@H](NC(=O)[C@@H](NC(=O)[C@H](CC1CCCCC1)NC(=O)c1cc(C(F)(F)F)c2ccccc2n1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccc2c(n1)CCCC2)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1cc2c([nH]1)CCCC2)C1CCCCC1)C(=O)N[C@@H](CCCNC(=N)N)C(=O)N[C@@H](CCCCN)C(N)=O]

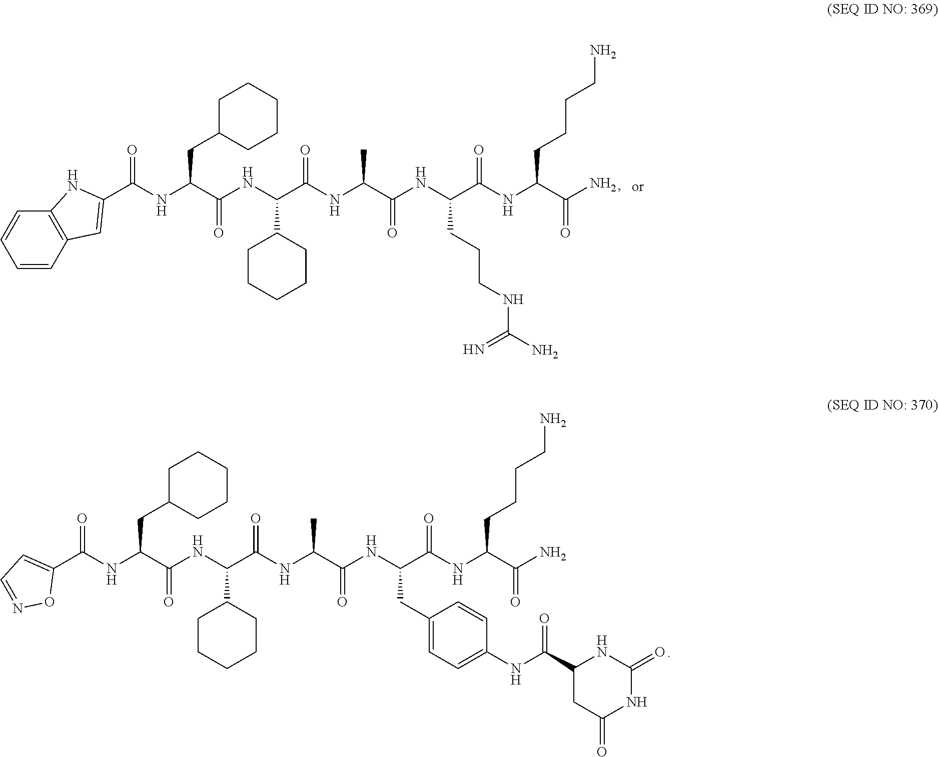

Any combination of the groups described above for the various variables is contemplated herein. Throughout the specification, groups and substituents thereof are chosen by one skilled in the field to provide stable moieties and compounds.

### Further Forms of Compounds

In one aspect, compounds described herein are in the form of pharmaceutically acceptable salts. In addition, the compounds described herein can exist in unsolvated as well as solvated forms with pharmaceutically acceptable solvents such as water, ethanol, and the like. The solvated forms of the compounds presented herein are also considered to be disclosed herein.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. See for example Handbook of Pharmaceutical Salts: Properties, Selection and Use, International Union of Pure and Applied Chemistry, Wiley-VCH 2002; S. M. Berge, L. D. Bighley, D. C. Monkhouse, J. Pharm. Sci. 1977, 66, 1-19, and P. H. Stahl and C. G. Wermuth, editors, Handbook of Pharmaceutical Salts: Properties, Selection and Use, Weinheim/Zürich:Wiley-VCH/VHCA, 2002, which are incorporated herein by reference. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I) with an acid. In some embodiments, the acid is an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to: 1-hydroxy-2-naphthoic acid; 2,2-dichloroacetic acid; 2-hydroxyethanesulfonic acid; 2-oxoglutaric acid; 4-acetamidobenzoic acid: 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid: dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (I), is prepared as a chloride salt, sulfate salt, bromide salt, mesylate salt, maleate salt, citrate salt or phosphate salt.

In some embodiments, pharmaceutically acceptable salts are obtained by reacting a compound of Formula (I), with a base. In some cases, compounds described herein coordinate with an organic base, such as, but not limited to, ethanolamine, diethanolamine, triethanolamine, tromethamine, meglumine, N-methylglucamine, dicyclohexylamine, or tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, any one of the hydrogen atoms on the organic radicals (e.g., alkyl groups, aromatic rings) of compounds described herein are replaced with deuterium.

In some embodiments, the compounds of Formula (I), possess one or more stereocenters and each stereocenter exists independently in either the R or S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds described herein, are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the formation of diastereomeric salts and separation by recrystallization, or chromatography, or any combination thereof. See for example Jean Jacques, Andre Collet, Samuel H. Wilen, “Enantiomers, Racemates and Resolutions”, John Wiley And Sons, Inc., 1981, which is incorporated herein by reference. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. See for example Design of Prodrugs, Bundgaard, A. Ed., Elsevier, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference.

A “metabolite” of a compound disclosed herein is a derivative of that compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

### Synthesis of Compounds

Compounds described herein are synthesized using standard synthetic techniques or using methods known in the art in combination with methods described herein.

Unless otherwise indicated, conventional methods of mass spectroscopy, NMR, and HPLC are employed.

Compounds are prepared using standard organic chemistry techniques such as those described in, for example, March's Advanced Organic Chemistry, 6^{th} Edition, John Wiley and Sons, Inc. Compounds may also be prepared using solid-phase peptide synthesis techniques such as those described in, for example, Solid Phase Peptide Synthesis, 2^{nd} Edition, The Pierce Chemical Co., Rockford, Ill. (1984). Alternative reaction conditions for the synthetic transformations described herein may be employed such as variation of solvent, reaction temperature, reaction time, as well as different chemical reagents and other reaction conditions.

### Peptide Synthesis

A peptide of the present disclosure may be prepared through known methods, including solid-phase peptide synthesis (SPPS). (See for example Palomo, Jose M. (2014). “Solid-phase peptide synthesis: An overview focused on the preparation of biologically relevant peptides” RSC Adv. 4 (62): 32658-32672; Krchňák, V; Holladay, Mark W. (2002). “Solid Phase Heterocyclic Chemistry” Chemical Reviews. 102 (1): 61-92; Merrifield, B. (1986-04-18). “Solid phase synthesis” Science. 232 (4748): 341-347; Guillier, F; et al., (2000). “Linkers and Cleavage Strategies in Solid-Phase Organic Synthesis and Combinatorial Chemistry.” Chemical Reviews. 100 (6): 2091-2158; Amblard M, et al., “Methods and protocols of modern solid phase Peptide synthesis.” Mol Biotechnol. 2006 July; 33(3):239-54).

In some embodiments, the solid-phase peptide synthesis is Fmoc solid-phase peptide synthesis. (See, for example, Behrendt, R., et al., (2016) Advances in Fmoc solid-phase peptide synthesis. J. Pept. Sci., 22: 4-27.

SPPS is a common technique for peptide synthesis. Usually, peptides are synthesized from the carbonyl group side (C-terminus) to amino group side (N-terminus) of the amino acid chain in the SPPS method, although peptides are biologically synthesized in the opposite direction in cells. In peptide synthesis, an amino-protected amino acid is bound to a solid phase material or resin (most commonly, low cross-linked polystyrene beads), forming a covalent bond between the carbonyl group and the resin, most often an amido or an ester bond. Then the amino group is deprotected and reacted with the carbonyl group of the next N-protected amino acid. The solid phase now bears a dipeptide. This cycle is repeated to form the desired peptide chain. After all reactions are complete, the synthesized peptide is cleaved from the bead.

The protecting groups for the amino groups mostly used in the peptide synthesis are 9-fluorenylmethyloxycarbonyl group (Fmoc) and t-butyloxycarbonyl (Boc). A number of amino acids bear functional groups in the side chain which must be protected specifically from reacting with the incoming N-protected amino acids. In contrast to Boc and Fmoc groups, these have to be stable over the course of peptide synthesis although they are also removed during the final deprotection of peptides.

An example solid-phase peptide synthesis may be carried out as follows. An esterification reaction occurs between the carboxyl group of a first amino acid (with a protected α-amino group) and the hydroxyl group of a hydroxyl-containing resin. The α-amino protecting group of the first amino acid is removed and a second amino acid is coupled with the first through its carboxyl group (all other functional groups are protected) to form a peptide bond between the first and second amino acids. The α-amino protecting group of the second amino acid is removed and a third amino acid is coupled with the second through its carboxyl group (all other functional groups are protected) to form a peptide bond between the second and third amino acids. These steps are repeated until the peptide of desired length is synthesized. Any remaining functional groups on the peptide chain are then deprotected. The peptide chain can then be cleaved from the resin.

Examples of resins used for SPPS include Merrifield resin, Rink amide resin, Wang resin, Sieber amide resin, MBHA resin, CTC resin, HMBA resin, DHP resin, and PAL resin. In some embodiments, the resin for SPPS is Rink amide resin. In some embodiments, the resin for SPPS is Wang resin. In some embodiments, the resin for SPPS is 2-chlorotrityl resin. In some embodiments, the resin for SPPS is Sieber amide resin.

Examples of α-amino protecting groups include benzyloxycarbonyl (Cbz), tertbutoxycarbonyl (Boc), fluorenylmethoxycarbonyl (Fmoc), and allyloxycarbonyl (Alloc) groups. In some embodiments, the α-amino protecting group is Fmoc. In some embodiments, the α-amino protecting group can be deprotected using acid, such as hydrofluoric acid or trifluoroacetic acid. In some embodiments, the α-amino protecting group can be deprotected using base, such as piperidine.

Examples of condensation agents used to activate a carboxyl group for an amidification or esterification reaction include HATU, DCC, EDC, BOP, and HBTU. In some embodiments, the condensation agent is HATU.

Examples of acids use to cleave a peptide chain from the resin include TFA.

In some embodiments, compounds are prepared as described in the Examples.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H A and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to, delivery via parenteral routes (including injection or infusion, and subcutaneous).

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and contain optional agents as excipients such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use.

### Methods of Treatment

In some embodiments, the methods comprise administering to a subject a therapeutically effective amount of a compound of Formula (I), or a pharmaceutically acceptable salt or solvate thereof. In some embodiments, the compound of Formula (I) or pharmaceutically acceptable salt or solvate thereof is administered in a pharmaceutical composition. In some embodiments, the subject has cancer. In some embodiments, the cancer is a solid tumor. In some embodiments, the subject has a noncancerous tumor. In some embodiments, the subject has an adenoma.

In some embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life, or combinations thereof.

In some embodiments, provided herein are methods for killing a tumor cell comprising contacting the tumor cell with a compound of Formula (I), or a pharmaceutically acceptable salt or solvate thereof. In some embodiments, the compound of Formula (I), or pharmaceutically acceptable salt or solvate thereof releases a number of alpha particles by natural radioactive decay. In some embodiments, the released alpha particles are sufficient to kill the tumor cell. In some embodiments, the released alpha particles are sufficient to stop cell growth. In some embodiments, the tumor cell is a malignant tumor cell. In some embodiments, the tumor cell is a benign tumor cell. In some embodiments, the method comprises killing a tumor cell with a beta-particle emitting radionuclide. In some embodiments, the method comprises killing a tumor cell with an alpha-particle emitting radionuclide. In some embodiments, the method comprises killing a tumor cell with a gamma-particle emitting radionuclide.

In one aspect, provided herein are methods and compositions for treating cancers. In some embodiments, the cancer is ovarian cancer. In some embodiments, the cancer is breast cancer. In some embodiments, the cancer is endometrial cancer. In some embodiments, the cancer is prostate cancer. In some embodiments, the cancer is renal cell carcinoma. In some embodiments, the cancer is lung cancer.

In one aspect, provided herein are methods and compositions for treating an adenoma.

In one aspect, provided herein are methods and compositions for treating a carcinoma.

In one aspect, provided herein is a method for identifying tumors in a mammal that overexpress PAR2 comprising: (i) administering to the mammal a PAR2 radiopharmaceutical described herein, or a pharmaceutically acceptable salt thereof; and (ii) performing single-photon emission computerized tomography (SPECT) or positron emission tomography (PET) analysis on the mammal. In some embodiments, the method comprises: (i) administering to the mammal a PAR2 radiopharmaceutical described herein, or a pharmaceutically acceptable salt thereof; and (ii) performing positron emission tomography (PET) analysis on the mammal.

In some embodiments, the mammal was diagnosed with cancer. In some embodiments, the mammal was diagnosed with ovarian cancer. In some embodiments, the mammal was diagnosed with breast cancer. In some embodiments, the mammal was diagnosed with endometrial cancer. In some embodiments, the mammal was diagnosed with prostate cancer. In some embodiments, the tissues in the mammal that overexpress PAR2 are tumors.

In some embodiments, a PAR2 radiopharmaceutical described herein, or a pharmaceutically acceptable salt thereof are used in a method for in vivo imaging of a subject. In some embodiments, the method includes the steps of:
(i) administering to the mammal PAR2 radiopharmaceutical described herein, or a pharmaceutically acceptable salt thereof;
(ii) waiting a sufficient amount of time to allow the PAR2 radiopharmaceutical, to accumulate at a tissue or cell site to be imaged; and
(iii) imaging the cells or tissues with a non-invasive imaging technique.

In some embodiments, the non-invasive imaging technique is single-photon emission computerized tomography (SPECT) or positron emission tomography (PET) analysis. In some embodiments, the non-invasive imaging technique is single-photon emission computerized tomography (SPECT). In some embodiments, the non-invasive imaging technique is selected from positron emission tomography imaging, or positron emission tomography with computed tomography imaging, and positron emission tomography with magnetic resonance imaging.

### Methods of Dosing and Treatment Regimens

In one embodiment, the PAR2 radiopharmaceutical described herein, or a pharmaceutically acceptable salt thereof, are used in the preparation of medicaments for the treatment of tumors in a mammal. Methods for treating any of the diseases or conditions described herein in a mammal in need of such treatment, involves administration of pharmaceutical compositions that include at least one compound of Formula (I), or a pharmaceutically acceptable salt thereof, in therapeutically effective amounts to said mammal.

In certain embodiments, the compositions containing the compound(s) described herein are administered for diagnostic and/or therapeutic treatments.

The amount of a given agent that corresponds to such an amount varies depending upon factors such as the particular conjugate, specific cancer or tumor to be treated (and its severity), the identity (e.g., weight, sex) of the subject or host in need of treatment, but nevertheless is determined according to the particular circumstances surrounding the case, including, e.g., the specific conjugate being administered, the route of administration, the condition being treated, and the subject or host being treated. Optimal doses are generally determined using experimental models and/or clinical trials. The optimal dose depends upon the body mass, weight, or blood volume of the subject.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans.

The amount of a compound of Formula (I), or pharmaceutically acceptable salts thereof, that are administered are sufficient to deliver a therapeutically effective dose to the particular subject. In some embodiments, dosages of a compound of Formula (I), are between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of a compound of Formula (I), or a pharmaceutically acceptable salt thereof described herein for methods of treating a disease as described herein is about 0.001 mg/kg to about I mg/kg body weight of the subject per dose. In some embodiments, the dose is about 0.001 mg to about 1000 mg per dose for the subject being treated. In some embodiments, a compound of Formula (I), or a pharmaceutically acceptable salt thereof described herein is administered to a subject at a dosage of from about 0.01 mg to about 500 mg, from about 0.01 mg to about 100 mg, or from about 0.01 mg to about 50 mg.

In some embodiments, a compound of Formula (I), or a pharmaceutically acceptable salt thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole.

In some embodiments, a compound of Formula (I), or a pharmaceutically acceptable salt thereof described herein is administered to a subject at a dosage of about 0.01 Gbq to about 1000 Gbq, about 0.5 Gbq to about 100 Gbq, or about 1 Gbq to about 50 Gbq.

In some embodiments, the dose is administered once a day, 1 to 3 times a week, I to 4 times a month, or 1 to 12 times a year.

In any of the aforementioned aspects are further embodiments in which the effective amount of the PAR2 radiopharmaceutical described herein, or a pharmaceutically acceptable salt thereof, is: (a) systemically administered to the mammal; and/or (b) intravenously administered to the mammal; and/or (c) administered by injection to the mammal.

In certain instances, it is appropriate to administer at least one PAR2 radiopharmaceutical described herein, or a pharmaceutically acceptable salt thereof, in combination with one or more other therapeutic agents.

### Certain Terminology

Unless otherwise stated, the following terms used in this application have the definitions given below. The use of the term “including” as well as other forms, such as “include,” “includes,” and “included,” is not limiting. The section headings used herein are for organizational purposes only and are not to be construed as limiting the subject matter described.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e., groups containing 1 carbon atom, 2 carbon atoms, 3 carbon atoms, or 4 carbon atoms, 5 carbon atoms or 6 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e., a —C₁-C₁₀ alkyl. Whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a —C₁-C₆ alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, the alkyl group is an “alkenyl” or “alkynyl” group.

An “alkylene” group refers to a divalent alkyl radical. Any of the above-mentioned monovalent alkyl groups may be an alkylene by abstraction of a second hydrogen atom from the alkyl. In some embodiments, an alkylene is a —C₁-C₆ alkylene. In other embodiments, an alkylene is a —C₁-C₄ alkylene. Typical alkylene groups include, but are not limited to: —CH₂—, —CH₂CH₂—, —CH₂CH₂CH₂—, —CH₂CH₂CH₂CH₂—, and the like. In some embodiments, an alkylene is —CH₂—. In some embodiments, an alkylene is —CH₂CH₂—.

An “alkoxy” group refers to an (alkyl)O— group, where alkyl is as defined herein.

The term “alkenyl” refers to a type of alkyl group in which at least one carbon-carbon double bond is present. In one embodiment, an alkenyl group has the formula: —C(R)═CR₂, wherein R refers to the remaining portions of the alkenyl group, which may be the same or different. In some embodiments, each R is independently H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portion of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, and —CH₂C≡CH.

The term “heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. In some embodiments, the “heteroalkyl” group has 2 to 10 atoms in the backbone, which include a combination of carbon atoms and heteroatoms (e.g. N, O, S), i.e., a 2 to 10-membered heteroalkyl. In some embodiments, the heteroalkyl is attached to the rest of the molecule at a carbon atom of the heteroalkyl. In one embodiment, a heteroalkyl is a 2 to 8 membered heteroalkyl.

A “heteroalkylene” group refers to a divalent alkyl radical derived from heteroalkyl, as exemplified, but not limited by, —CH₂—CH₂—O—CH₂—CH₂— and —CH₂—O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(═O)O— represents both —C(═O)O— and —OC(═O)—. Additionally, the formula —C(═O)NH-represents both —C(═O)NH— and —NHC(═O)—.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom that is different from carbon. In some embodiments, carbocycles are monocyclic, bicyclic, or multicyclic. In some embodiments, a carbocycle is a monocyclic carbocycle or a bicyclic carbocycle. A bicyclic carbocycle is a fused bicyclic carbocycle, bridged bicyclic carbocycle, or spirocyclic bicyclic carbocycle. A bicyclic carbocycle is a fused bicyclic carbocycle, bridged bicyclic carbocycle, or spirocyclic bicyclic carbocycle. The term “fused bicyclic carbocycle” refers to two carbocycles that are joined together and share two adjacent carbon atoms. The term “bridged bicyclic carbocycles” refers to two carbocycles that are joined together and share three or more carbon atoms. The term “spirocyclic carbocycles” or a “spiro bicyclic carbocycles” refers to two carbocycles that are joined together and share a single carbon atom.

A carbocycle is fully saturated (i.e., no units of unsaturation, also known as cycloalkyl), partially unsaturated (i.e., at least one unit of unsaturation, i.e., cycloalkenyl), or fully unsaturated (i.e., aryl). Carbocycles include aryls and cycloalkyls.

In some embodiments, at least one of the two rings of a partially unsaturated bicyclic carbocycle is aromatic. In some embodiments, both rings of a bicyclic carbocycle are aromatic. In some embodiments, partially unsaturated bicyclic carbocycles include indanyl

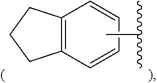

indenyl

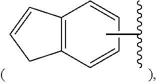

and tetrahydronaphthyl

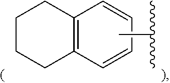

1,4-dihydronaphthalene, and 1,2-dihydronaphthalene. In some other embodiments, partially unsaturated bicyclic carbocycles include fused bicyclic cycloalkenyl, spirocyclic cycloalkenyl and bridged bicyclic cycloalkenyl.

As used herein, the term “aryl” refers to an aromatic ring wherein each of the atoms forming the ring is a carbon atom. In one aspect, aryl is phenyl

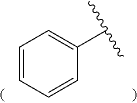 [SMILES: *c1ccccc1]

or naphthyl

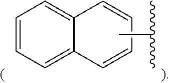

A monocyclic aryl is phenyl. A bicyclic aryl is naphthyl. In some embodiments, an aryl is a C₆-C₁₀ aryl. Depending on the structure, an aryl group is a monoradical or a diradical (i.e., an arylene group).

The term “cycloalkyl” refers to a monocyclic, bicyclic, or polycyclic aliphatic, non-aromatic radical, wherein each of the atoms forming the ring (i.e., skeletal atoms) is a carbon atom. In some embodiments, a cycloalkyl is a fused cycloalkyl, spirocyclic cycloalkyl, or bridged cycloalkyl. Cycloalkyl groups include groups having from 3 to 12 ring atoms. In some embodiments, cycloalkyl groups are selected from cyclopropyl, cyclobutyl, cyclopentyl, cyclopentenyl, cyclohexyl, cyclohexenyl, cycloheptyl, cyclooctyl, spiro[2.2]pentyl, and bicyclo[1.1.1]pentyl. In some embodiments, a cycloalkyl is a C₃-C₆ cycloalkyl. In some embodiments, a cycloalkyl is a C₃-C₄ cycloalkyl. In some embodiments, a cycloalkyl is a C₅-C₆ cycloalkyl. Non-limiting examples of fused bicyclic cycloalkyl include: bicyclo[4.4.0]decane, bicyclo[4.3.0]nonane, bicyclo[4.2.0]octane, bicyclo[4.1.0]heptane, bicyclo[3.3.0]octane, or bicyclo[3.1.0]hexane. Non-limiting examples of spirocyclic cycloalkyls include: spiro[2.2]pentyl, spiro[3.2]hexyl, spiro[3.3]heptyl, spiro[4.3]octyl, spiro[4.2]heptyl, spiro[5.3]nonyl, spiro[5.4]decanyl, spiro[5.5]undecanyl, spiro[5.2]octyl, spiro[6.3]decanyl, spiro[6.4]undecanyl, or spiro[6.5]dodecanyl. Non-limiting examples of bridged bicyclic cycloalkyl include: bicyclo[1.1.1]pentanyl, bicyclo[2.1.1]hexanyl, bicyclo[3.1.1]heptanyl, bicyclo[2.2.1]heptanyl, bicyclo[2.2.2]octanyl, bicyclo[3.2.2]octanyl, bicyclo[3.3.1]nonanyl, bicyclo[4.2.2]dodecanyl, bicyclo[3.3.2]dodecanyl, bicyclo[3.2.2]nonanyl, bicyclo[3.3.3]undecanyl, or bicyclo[3.1.1]heptanyl.

The term “cycloalkenyl” refers to a type of non-aromatic cycloalkyl group in which at least one carbon-carbon double bond is present. In some embodiments, cycloalkenyl is a monocyclic cycloalkenyl or bicyclic cycloalkenyl. In some embodiments, cycloalkenyl is a monocyclic cycloalkenyl. In some embodiments, cycloalkenyl is a bicyclic cycloalkenyl. In some embodiments, a bicyclic cycloalkenyl is a fused bicyclic cycloalkenyl, spirocyclic cycloalkenyl, or a bridged bicyclic cycloalkenyl.

Non-limiting examples of monocyclic cycloalkenyls include: cyclopentenyl, cyclohexenyl, cycloheptenyl, or cyclooctenyl. In some embodiments, monocyclic cycloalkenyls include:

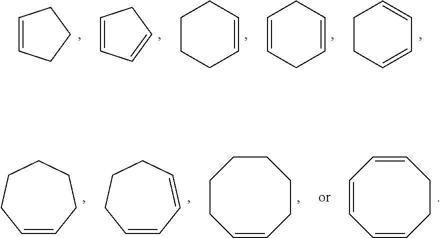

Non-limiting examples of spirocyclic cycloalkenyls include:

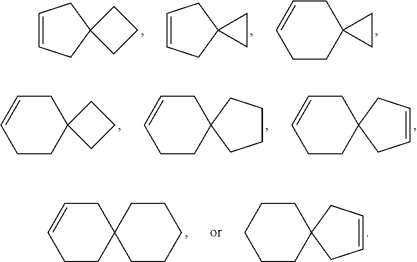

Non-limiting examples of fused bicyclic cycloalkenyl include:

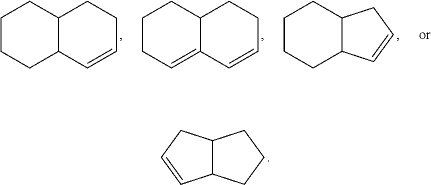

Non-limiting examples of bridged bicyclic cycloalkenyl include:

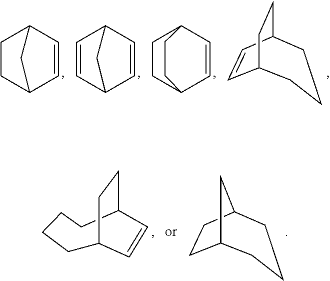

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a —C₁-C₆ fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 12 atoms in its ring system, and with the proviso that any ring does not contain two adjacent O or S atoms. Non-aromatic heterocyclic groups (also known as heterocycloalkyls) can be fully saturated (i.e., no units of unsaturation, also known as heterocycloalkyl), partially unsaturated (i.e., at least one unit of unsaturation), or fully unsaturated (i.e., heteroaryl). In some embodiments, fully saturated or partially unsaturated heterocyclic groups include rings having 3 to 12 atoms in its ring system and aromatic heterocyclic groups include rings having 5 to 10 atoms in its ring system. In some embodiments, at least one of the two rings of a partially unsaturated bicyclic heterocycle is aromatic. The heterocyclic groups include partially unsaturated bicyclic heterocyclic rings wherein the heterocycloalkyl is fused with an aryl or heteroaryl. In some embodiments, one of the two rings of a bicyclic partially unsaturated heterocycle is aromatic, and the point of attachment to the remaining of the compound is at a carbon atom or heteroatom of the aromatic ring. In some embodiments, one of the two rings of a bicyclic heterocycle is aromatic, and the point of attachment to the remaining of the compound is at a carbon atom or heteroatom of the non-aromatic ring. In some embodiments, bicyclic heterocycle is a fused bicyclic heterocycle, bridged bicyclic heterocycle, or a spirocyclic bicyclic heterocycle. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl

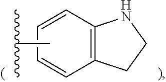

isoindolinyl

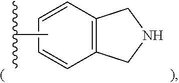

2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H′)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). Examples of partially unsaturated bicyclic heterocyclic rings are indolinyl

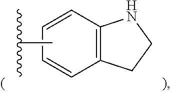

isoindolinyl

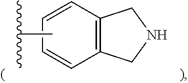

1,2-dihydroisoquinolinyl

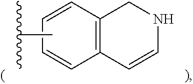

1,2,3,4-tetrahydroquinolinyl

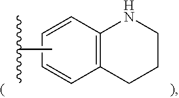

1,2,3,4-tetrahydroisoquinolinyl

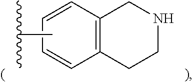

1,2,3,4-tetrahydroquinoxalinyl

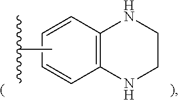

1,2,3,4-tetrahydro-1,8-naphthyridine

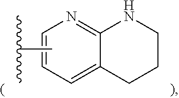

5,6,7,8-tetrahydro-1,7-naphthyridinyl

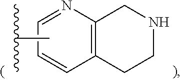

5,6,7,8-tetrahydro-1,6-naphthyridinyl

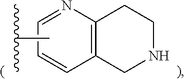

5,6,7,8-tetrahydro-1,5-naphthyridinyl

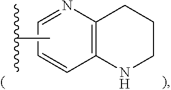

1,2,3,4-tetrahydro-1,6-naphthyridinyl

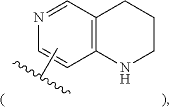

1,2,3,4-tetrahydro-2,6-naphthyridinyl

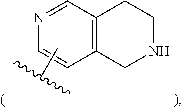

1,2,3,4-tetrahydro-3,6-naphthyridinyl

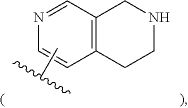

1,2,3,4-tetrahydro-4,6-naphthyridinyl

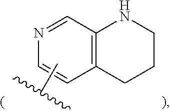

or 2,3-dihydrobenzo[d]oxazole

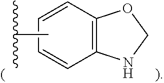

Partially unsaturated bicyclic heterocyclic groups are optionally substituted with one or two oxo (═O) moieties, such as pyrrolidin-2-one, indolin-2-one

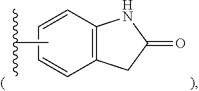

isoindolin-1-one

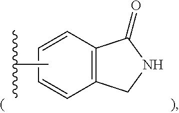

benzo[d]oxazol-2(3H)-one

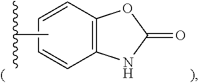

3,4-dihydroquinolin-2(1H)-one

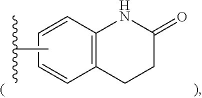

or 3,4-dihydroisoquinolin-1(2H)-one

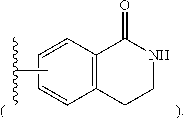

The terms “heteroaryl” or, alternatively, “heteroaromatic” refer to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizinyl, indolyl, benzofuranyl, benzothiophenyl, indazolyl, benzimidazolyl, purinyl, quinolizinyl, quinolinyl, isoquinolinyl, cinnolinyl

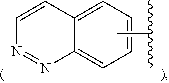

phthalazinyl

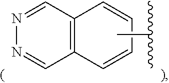

quinazolinyl

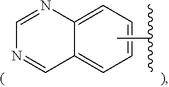

quinoxalinyl

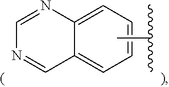

1,8-naphthyridinyl

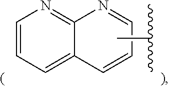

1,7-naphthyridinyl

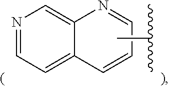

1,6-naphthyridinyl

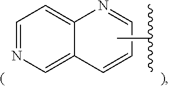

1,5-naphthyridinyl

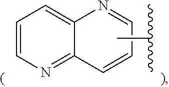

pteridinyl

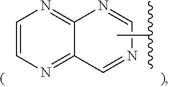

and tetrazolo[1,5-a]pyridinyl

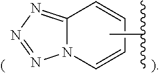

In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, a heteroaryl contains 1 O atom. In some embodiments, a heteroaryl contains I S atom in the ring. In some embodiments, a heteroaryl is a 5 to 10-membered heteroaryl. In some embodiments, a monocyclic heteroaryl is a 5 to 6 membered heteroaryl. In some embodiments, a monocyclic heteroaryl is a 5-membered heteroaryl. In some embodiments, a monocyclic heteroaryl is a 6-membered heteroaryl. In some embodiments, a bicyclic heteroaryl is a 10-membered heteroaryl.

A “heterocycloalkyl” group refers to a cycloalkyl group that includes at least one heteroatom selected from nitrogen, oxygen and sulfur. A “heterocycloalkyl” group is optionally substituted with one or two oxo (═O) moieties, such as pyrrolidin-2-one. In some embodiments, heterocycloalkyl is a monocyclic heterocycloalkyl or bicyclic heterocycloalkyl. In some embodiments, heterocycloalkyl is a monocyclic heterocycloalkyl. In some embodiments, heterocycloalkyl is a bicyclic heterocycloalkyl. In some embodiments, the bicyclic cycloalkyl is a spiro bicyclic (i.e., spirocyclic) heterocycloalkyl, a fused bicyclic heterocycloalkyl, or a bridged bicyclic heterocycloalkyl. In one aspect, a heterocycloalkyl is a 3 to 12-membered heterocycloalkyl. In another aspect, a heterocycloalkyl is a 5 to 10-membered heterocycloalkyl. In some embodiments, a heterocycloalkyl is a 5-membered heterocycloalkyl. In some embodiments, a heterocycloalkyl is a 6-membered heterocycloalkyl. In some embodiments, a heterocycloalkyl is monocyclic or bicyclic. In some embodiments, a heterocycloalkyl is a 10-membered bicyclic heterocycloalkyl. In some embodiments, a heterocycloalkyl is a 9-membered bicyclic heterocycloalkyl. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1-4 nitrogen (N) atoms in the ring. In some embodiments, a heterocycloalkyl contains 0-2 N atoms, 0-2 oxygen (O) atoms and 0-1 sulfur (S) atoms in the ring.

Monocyclic heterocycloalkyls that include 2 to 10 carbon atoms in the ring include aziridinyl, azetidinyl, pyrrolidinyl, piperidinyl, azepanyl, oxetanyl, thietanyl, thioxanyl, oxazolidinonyl, dihydrofuranyl, tetrahydrofuranyl, tetrahydrothienyl, dihydropyranyl, tetrahydropyranyl, 2H-pyranyl, 4H-pyranyl, tetrahydrothiopyranyl, morpholinyl, thiomorpholinyl, piperazinyl, homopiperidinyl, oxepanyl, thiepanyl, oxazepinyl, diazepinyl, thiazepinyl, 1,2,3,6-tetrahydropyridinyl, piperidin-2-onyl, pyrrolidine-2,5-dithionyl, pyrrolidine-2,5-dionyl, pyrrolidinonyl, imidazolidinyl, imidazolidin-2-onyl, thiazolidin-2-onyl, pyrrolin-2-yl, pyrrolin-3-yl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, or imidazolidinyl. In some embodiments, monocyclic heterocycloalkyls that include 2 to 8 carbon atoms in the ring include aziridinyl, azetidinyl, pyrrolidinyl, piperidinyl, azepanyl, oxetanyl, tetrahydrofuranyl, tetrahydrothienyl, tetrahydropyranyl, morpholinyl, thiomorpholinyl, piperazinyl, or homopiperidinyl, In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

Non-limiting examples of spirocyclic heterocycloalkyls include:

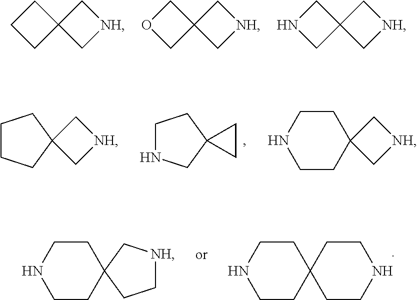

Non-limiting examples of fused bicyclic heterocycloalkyl include:

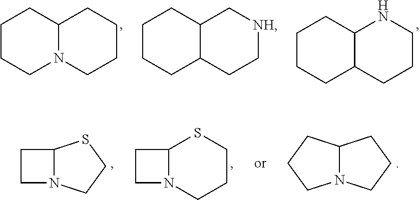

Non-limiting examples of bridged bicyclic heterocycloalkyl include:

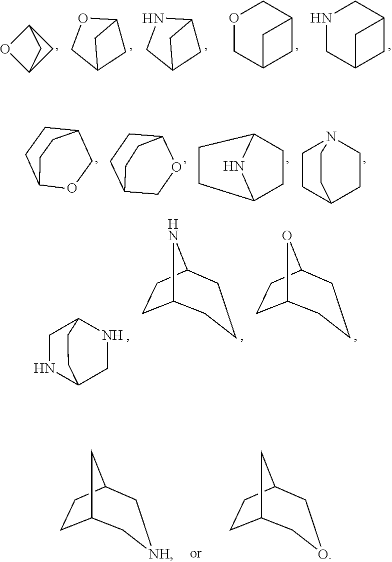

The term “bond” or “single bond” refers to a chemical bond between two atoms, or two moieties when the atoms joined by the bond are considered to be part of a larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄alkyl), —S(═O)₂N(C₁-C₄ alkyl)₂, —C₁-C₄ alkyl, —C₃-C₆ cycloalkyl, —C₁-C₄ fluoroalkyl, —C₁-C₄ heteroalkyl, —C₁-C₄ alkoxy, —C₁-C₄ fluoroalkoxy, —SC₁-C₄ alkyl, —S(═O)C₁-C₄alkyl, and —S(═O)₂C₁-C₄ alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion). Those of skill in the art are familiar with administration techniques that can be employed with the compounds and methods described herein.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

Chemical structures depicted herein include all stereochemical forms of the structure, unless otherwise stated.

The term “peptide” as used herein refers to a compound comprising two or more amino acids in a serial array, linked through peptide bonds. The amino acids making up the polypeptide may be naturally derived, or may be synthetic.

The term “amino acid” as used herein refers to both natural and unnatural amino acids. The term “unnatural amino acid” as used herein refers to an amino acid that is not part of the 20 amino acids that occur naturally in protein.

As used herein, amino acid residue refers to an amino acid formed upon chemical digestion (hydrolysis) of a polypeptide at its peptide linkages. The amino acid residues described herein are, in certain embodiments, in the “L” isomeric form. Residues in the “D” isomeric form can be substituted for any “L” amino acid residue, as long as the desired functional property is retained by the polypeptide. “—NH₂” refers to the free amino group present at the amino terminus of a polypeptide. “—CO₂H” refers to the free carboxy group present at the carboxyl terminus of a polypeptide. In keeping with standard polypeptide nomenclature described in J. Biol. Chem., 243:3552 59 (1969) and adopted at 37 C.F.R. §§ 1.821-1.822, abbreviations for amino acid residues are shown in the following Table B:

TABLE B
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino terminus to carboxyl terminus. In addition, the phrase “amino acid residue” is broadly defined to include the amino acids listed in the Table of Correspondence and modified and unusual amino acids, Such as those referred to in 37 C.F.R. §§ 1.8.21-1.822, and incorporated herein by reference. Furthermore, it should be noted that a dash at the beginning or end of an amino acid residue sequence indicates a peptide bond to a further sequence of one or more amino acid residues or to an amino terminal group such as —NH₂ or to a carboxyl terminal group such as —CO₂H.

In a peptide, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in Table C as follows:

TABLE C

| Original residue | Conservative substitution |
|---|---|
| Ala (A) | Gly; Ser |
| Arg (R) | Lys |
| Asn (N) | Gln; His |
| Asp (D) | Glu |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Representative amino acid side chains are shown in Table D.

TABLE D
Representative amino acid side chains

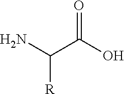

R
—H
Glycine (Gly)

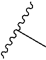

Alanine (Ala)

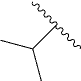 [SMILES: *C(C)C]

Valine (Val)

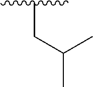 [SMILES: *CC(C)C]

Leucine (Leu)

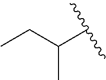 [SMILES: *C(C)CC]

Isoleucine (Ile)

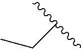 [SMILES: *CC]

Homoalanine (HAla)

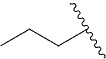 [SMILES: *CCC]

Norvaline (Nva)

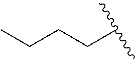 [SMILES: *CCCC]

Norleucine (Nle)

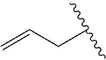 [SMILES: *CC=C]

Allylglycine (Allylgly)

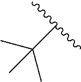 [SMILES: *C(C)(C)C]

tert-Leucine (Tle)

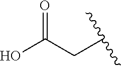 [SMILES: *CC(=O)O]

Aspartic Acid (Asp)

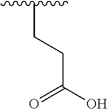 [SMILES: *CCC(=O)O]

Glutamic acid (Glu)

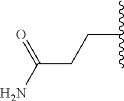 [SMILES: *CCC(N)=O]

Glutamine (Gln)

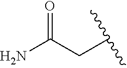 [SMILES: *CC(N)=O]

Asparagine (Asn)

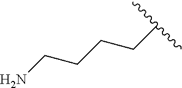 [SMILES: *CCCCN]

Lysine (Lys)

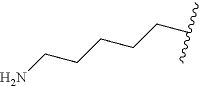 [SMILES: *CCCCCN]

Homolysine (HLys)

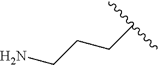 [SMILES: *CCCN]

Ornithine (Orn)

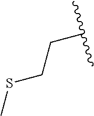 [SMILES: *CCSC]

Methionine (Met)

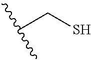 [SMILES: *CS]

Cysteine (Cys)

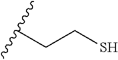 [SMILES: *CCS]

Homocysteine (HCys)

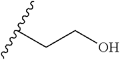 [SMILES: *CCO]

Homoserine (HSer)

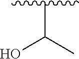 [SMILES: *C(C)O]

Threonine (Thr)

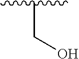 [SMILES: *CO]

Serine (Ser)

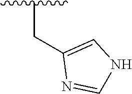 [SMILES: *Cc1c[nH]cn1]

Histidine (His)

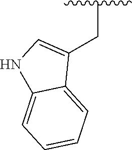 [SMILES: *Cc1c[nH]c2ccccc12]

Tryptophan (Trp)

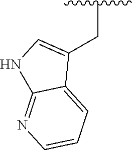 [SMILES: *Cc1c[nH]c2ncccc12]

7-aza-Trp

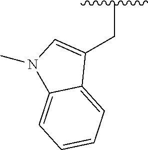 [SMILES: *Cc1cn(C)c2ccccc12]

1-methyltryptophan (1MT)

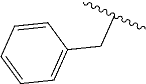 [SMILES: *Cc1ccccc1]

Phenylalanine (Phe)

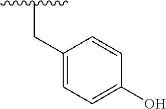 [SMILES: *Cc1ccc(O)cc1]

Tyrosine (Tyr)

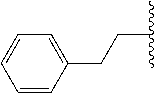 [SMILES: *CCc1ccccc1]

Homophenylalanine (HPhe)

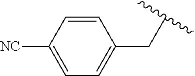 [SMILES: *Cc1ccc(C#N)cc1]

4-cyano phenylalanine
(Phe(4-CN))

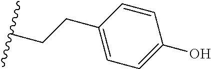 [SMILES: *CCc1ccc(O)cc1]

Homotyrosine (HTyr)

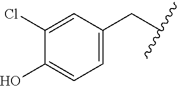 [SMILES: *Cc1ccc(O)c(Cl)c1]

3-chlorotyrosine (Tyr(3-Cl))

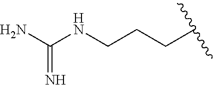 [SMILES: *CCCNC(=N)N]

Arginine (Arg)

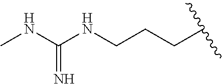 [SMILES: *CCCNC(=N)NC]

N^{ω}-methylarginine

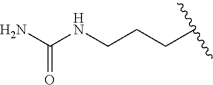 [SMILES: *CCCNC(N)=O]

Citrulline (Cit)

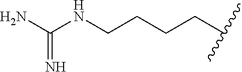 [SMILES: *CCCCNC(=N)N]

Homoarginine (HArg)

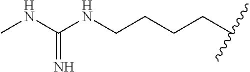 [SMILES: *CCCCNC(=N)NC]

Methyl homoarginine (homo-
Arg(Me))

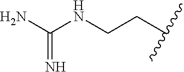 [SMILES: *CCNC(=N)N]

Norarginine (AGBA)

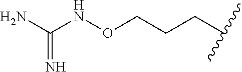 [SMILES: *CCCONC(=N)N]

Canavanine

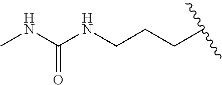 [SMILES: *CCCNC(=O)NC]

Methyl Citrulline (Cit(Me))

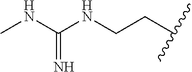 [SMILES: *CCNC(=N)NC]

Methyl Norarginine
(AGBA(Me))

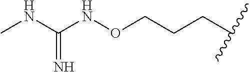 [SMILES: *CCCONC(=N)NC]

Methyl Canavanine

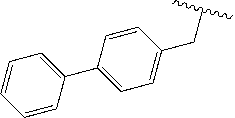 [SMILES: *Cc1ccc(-c2ccccc2)cc1]

Biphenylalanine (Bip)

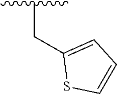 [SMILES: *Cc1cccs1]

β-(2-thienyl)-Ala

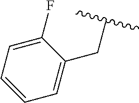 [SMILES: *Cc1ccccc1F]

2-Fluorophenylalanine
(Phe(2-F))

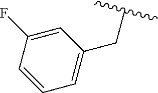 [SMILES: *Cc1cccc(F)c1]

3-Fluorophenylalanine
(Phe(3-F))

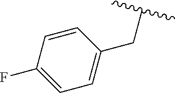 [SMILES: *Cc1ccc(F)cc1]

4-Fluorophenylalanine (Phe(4-
F))

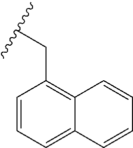 [SMILES: *Cc1cccc2ccccc12]

3-(1-Naphthyl)alanine (α-
Nal)

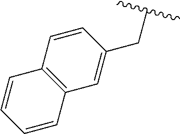 [SMILES: *Cc1ccc2ccccc2c1]

3-(2-Naphthyl)alanine or
(β-Nal or 2Nal)

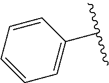 [SMILES: *c1ccccc1]

Phenylglycine (Phg)

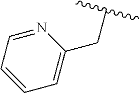 [SMILES: *Cc1ccccn1]

(pyridin-2-yl)alanine or 3-(2-
Pyridyl)alanine (2-Pal)

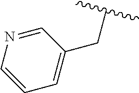 [SMILES: *Cc1cccnc1]

(pyridin-3-yl)alanine or 3-(3-
Pyridyl)alanine (3-Pal)

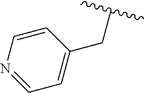 [SMILES: *Cc1ccncc1]

(pyridin-4-yl)alanine or 3-(4-
Pyridyl)alanine (4-Pal)

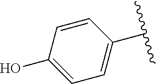 [SMILES: *c1ccc(O)cc1]

4-hydroxyphenylglycine
(Phg(4-OH))

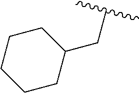 [SMILES: *CC1CCCCC1]

Cyclohexylalanine (Cha)

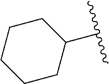 [SMILES: *C1CCCCC1]

Cyclohexylglycine (Chg)

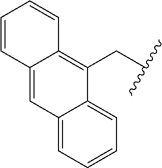 [SMILES: *Cc1c2ccccc2cc2ccccc12]

3-(9-anthryl)-alanine
(H-Ala(9-Anth)-OH, AAP)

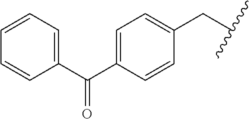 [SMILES: *Cc1ccc(C(=O)c2ccccc2)cc1]

4-Benzoyl-L-phenylalanine
(Bpa)

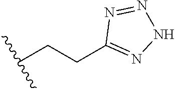 [SMILES: *CCc1nn[nH]n1]

(S)-2-amino-4-(2H-tetrazol-5-
yl)butanoic acid

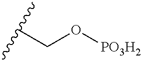

O-phospho-serine (SOP)

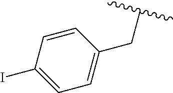 [SMILES: *Cc1ccc(I)cc1]

4-Iodophenylalanine
(Phe(4-I))

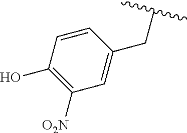 [SMILES: *Cc1ccc(O)c([N+](=O)[O-])c1]

3-nitro-tyrosine
Tyr(3-NO₂)

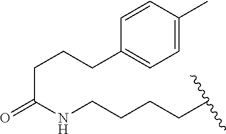 [SMILES: *CCCCNC(=O)CCCc1ccc(C)cc1]

N6-(4-(p-tolyl)butanoyl)-
lysine

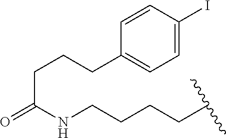 [SMILES: *CCCCNC(=O)CCCc1ccc(I)cc1]

N6-(4-(4-iodophenyl)
butanoyl)-lysine

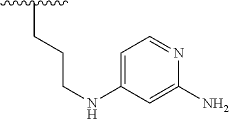 [SMILES: *CCCNc1ccnc(N)c1]

2-((2-aminopyridin-4-yl)-λ²-
azaneyl)ethyl alanine

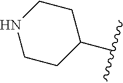 [SMILES: *C1CCNCC1]

2-(piperidin-4-yl)-glycine
(Gly(piperidin-4-yl)) or 2-
amino-2-(piperidin-4-
yl)acetic

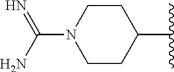 [SMILES: *C1CCN(C(=N)N)CC1]

2-(1-carbamimidoylpiperidin-4-
yl)-glycine (Gly(1-
carbamimidoylpiperidin-4-yl))

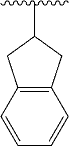 [SMILES: *C1Cc2ccccc2C1]

2-indanylglycine or 2-amino-
2-(2,3-dihydro-1H-inden-2-
yl)acetic acid

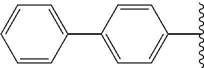 [SMILES: *c1ccc(-c2ccccc2)cc1]

Biphenylglycine

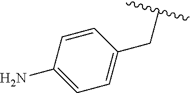 [SMILES: *Cc1ccc(N)cc1]

4-aminophenylalanine
(Phe(4-NH₂))

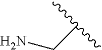 [SMILES: *CN]

2,3-diaminopropanoic acid or
3-amino-alanine (H-Dap-OH)

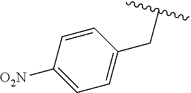 [SMILES: *Cc1ccc([N+](=O)[O-])cc1]

4-nitrophenylalanine
(Phe(4-NO₂))

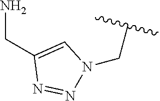 [SMILES: *Cn1cc(CN)nn1]

2-((4-(aminomethyl)-1H-
1,2,3-triazol-1-yl)methyl)-
glycine

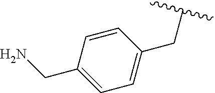 [SMILES: *Cc1ccc(CN)cc1]

4-aminomethylphenylalanine
(Phe(4-CH₂NH₂))

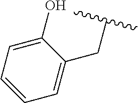 [SMILES: *Cc1ccccc1O]

2-hydroxyphenylalanine
(Phe(2-OH))

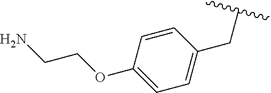 [SMILES: *Cc1ccc(OCCN)cc1]

4-(2-aminoethoxy)
phenylalanine (Phe(4-
OCH₂CH₂NH₂))

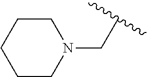 [SMILES: *CN1CCCCC1]

piperidinyl-1-yl alanine

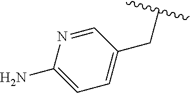 [SMILES: *Cc1ccc(N)nc1]

3-(6-aminopyridin-3-
yl)alanine ((3-Pal(6-NH₂))

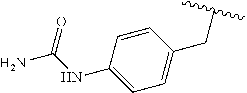 [SMILES: *Cc1ccc(NC(N)=O)cc1]

4-ureidophenylalanine

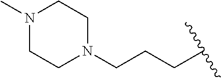 [SMILES: *CCCN1CCN(C)CC1]

3-((4-methylpiperazin-1-
yl)propyl)-glycine

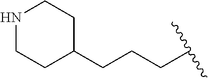 [SMILES: *CCCC1CCNCC1]

2-(piperidin-4-
yl)ethylalanine or 2-amino-5-
(piperidin-4-yl)pentanoic
acid

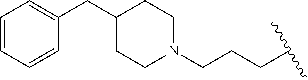 [SMILES: *CCCN1CCC(Cc2ccccc2)CC1]

3-(4-benzylpiperidin-1-
yl)propylglycine

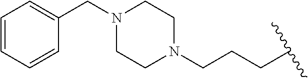 [SMILES: *CCCN1CCN(Cc2ccccc2)CC1]

3-((4-benzylpiperazin-1-
yl)propyl)-glycine

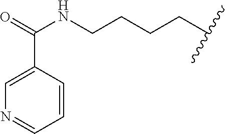 [SMILES: *CCCCNC(=O)c1cccnc1]

N6-nicotinoyl-Lys

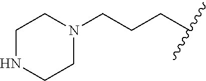 [SMILES: *CCCN1CCNCC1]

3-((piperazin-1-yl)propyl)-
glycine

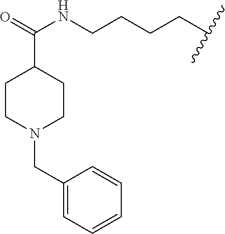 [SMILES: *CCCCNC(=O)C1CCN(Cc2ccccc2)CC1]

N6-(1-benzylpiperidine-4-
carbonyl)-Lys

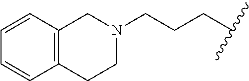 [SMILES: *CCCN1CCc2ccccc2C1]

3-((3,4-dihydroisoquinolin-
2(1H)-yl)propyl)-glycine

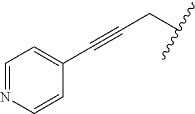 [SMILES: *CC#Cc1ccncc1]

2-pyridin-4-ylthynyl alanine

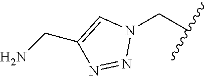 [SMILES: *Cn1cc(CN)nn1]

2-((4-(aminomethyl)-1H-
1,2λ²,3λ²-triazol-1-yl)methyl)-
glycine

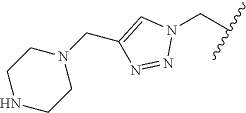 [SMILES: *Cn1cc(CN2CCNCC2)nn1]

4-(piperazin-1-ylmethyl)-1H-
1,2λ²,3λ²-triazol-1-
yl)methyl)-glycine

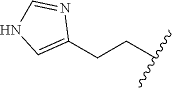 [SMILES: *CCc1c[nH]cn1]

(S)-2-((1H-imidazol-4-
yl)ethyl)glycine

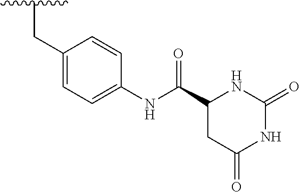

(4-((R)-2,6-
dioxohexahydropyrimidine-4-
carboxamido)phenyl)alanine

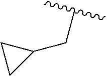 [SMILES: *CC1CC1]

cyclopropyl alanine

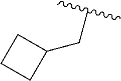 [SMILES: *CC1CCC1]

3-cyclobutyl alanine

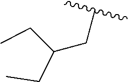 [SMILES: *CC(CC)CC]

4-ethyl-norleucine or
2-amino-4-ethylhexanoic acid

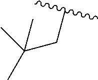 [SMILES: *CC(C)(C)C]

3-tert-butyl-alanine or 2-
amino-4,4-dimethylpentanoic
acid

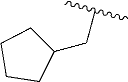 [SMILES: *CC1CCCC1]

3-cyclopentylalanine (Cpa-
OH) or 2-amino-3-
cyclopentylpropanoic acid

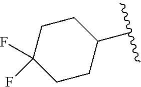 [SMILES: *C1CCC(F)(F)CC1]

4,4-difluorocyclohexylglycine

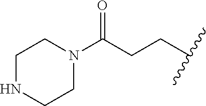 [SMILES: *CCC(=O)N1CCNCC1]

2-oxo-2-(piperazin-1-yl)ethyl
alanine or 2-amino-5-oxo-5-
(piperazin-1-yl)pentanoic
acid

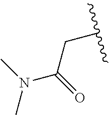 [SMILES: *CC(=O)N(C)C]

N⁴,N⁴-dimethyl-asparagine
(N⁴,N⁴-dimethyl-Asn)

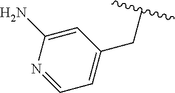 [SMILES: *Cc1ccnc(N)c1]

(S)-3-(6-aminopyridin-4-
ylalanine ((4-Pal(6-NH₂))

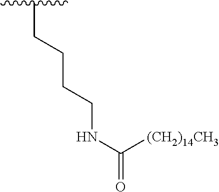

N6-palmitoyl-Lys

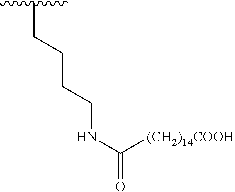

N6-(15-
carboxypentadecanoyl)-Lys

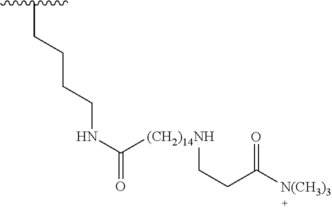

N6-(16-oxo-16-((2-(N-N-N-
trimethyl-amino)ethyl)amino)
hexadecanoyl)-Lys

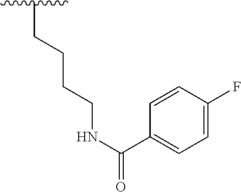 [SMILES: *CCCCNC(=O)c1ccc(F)cc1]

N6-(4-fluorobenzoyl)-Lys

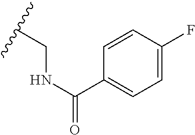 [SMILES: *CNC(=O)c1ccc(F)cc1]

(4-fluorobenzamido)
methylglycine

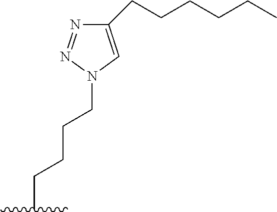 [SMILES: *CCCCn1cc(CCCCCC)nn1]

2-amino-6-(4-hexyl-1H-1,2,3-
triazol-1-yl)hexanoic acid

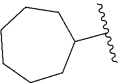 [SMILES: *C1CCCCCC1]

Cycloheptylglycine

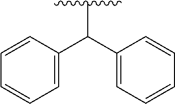 [SMILES: *C(c1ccccc1)c1ccccc1]

3,3-diphenyl-alanine (Dip)

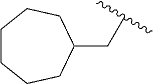 [SMILES: *CC1CCCCCC1]

cycloheptylalanine

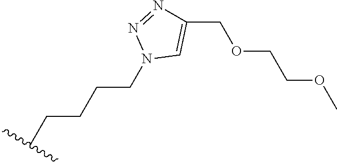 [SMILES: *CCCCn1cc(COCCOC)nn1]

2-amino-6-(4-((2-
methoxyethoxy)methyl)-1H-
1,2,3-triazol-1-yl)hexanoic
acid

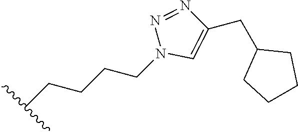 [SMILES: *CCCCn1cc(CC2CCCC2)nn1]

2-amino-6-(4-
(cyclopentylmethyl)-1H-
1,2,3-triazol-1-yl)hexanoic
acid

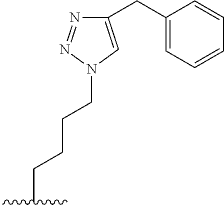 [SMILES: *CCCCn1cc(Cc2ccccc2)nn1]

2-amino-6-(4-benzyl-1H-
1,2,3-triazol-1-yl)hexanoic
acid

Additional amino acids are shown in Tables E and F.

TABLE E
Representative cyclic and unnatural amino acids

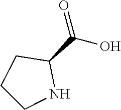

Proline (Pro)

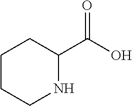

Piperidine-2-carboxylic acid

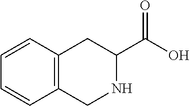 [SMILES: O=C(O)C1Cc2ccccc2CN1]

2,3,4,5-
Tetrahydroisoquinoline-3-
carboxylic acid (Tic)

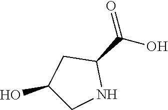

Hydroxyproline (Hyp)

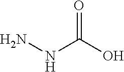

Azaglycine (AzaGly)

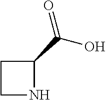

Azetidine-2-carboxylic acid
(Aze)

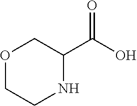

Morpholine-2-carboxylic acid

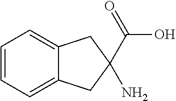 [SMILES: NC1(C(=O)O)Cc2ccccc2C1]

2-Amino-2-indancarboxylic
acid (Aic)

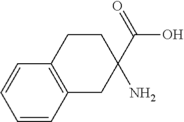 [SMILES: NC1(C(=O)O)CCc2ccccc2C1]

2-aminotetralin-2-
carboxylic acid (Atc)

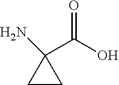

1-Aminocyclopropane-1-
carboxylic acid (ACC)

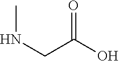

Sarcosine (Sar)

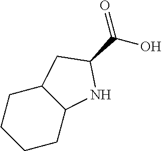 [SMILES: O=C(O)[C@@H]1CC2CCCCC2N1]

Octahydroindole-2-
carboxylic acid (Oic)

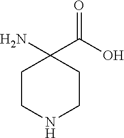

4-aminopiperidine-4-carboxylic
acid

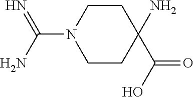 [SMILES: N=C(N)N1CCC(N)(C(=O)O)CC1]

4-amino-1-
carbamimidoylpiperidine-4-
carboxylic acid

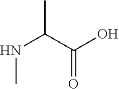

N-methylalanine
(N-Me-Ala)

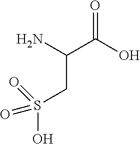

3-sulfo-alanine (Ala-SO₃H)

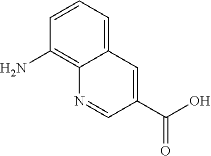 [SMILES: Nc1cccc2cc(C(=O)O)cnc12]

8-aminoquinoline-3-
carboxylic acid

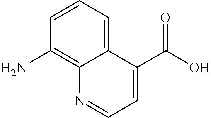 [SMILES: Nc1cccc2c(C(=O)O)ccnc12]

8-aminoquinoline-4-
carboxylic acid

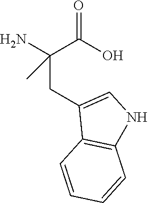 [SMILES: CC(N)(Cc1c[nH]c2ccccc12)C(=O)O]

Methyl-tryptophan (Me-Trp)

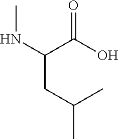

Methyl-leucine (Me-Leu)

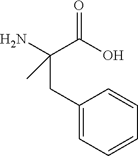

Methyl-phenylalanine (Me-
Phe)

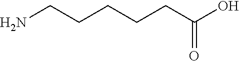

6-aminohexanoic acid (Ahx)

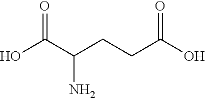

γ-Glutamic acid (γ-Glu)

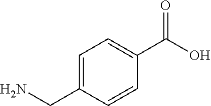 [SMILES: NCc1ccc(C(=O)O)cc1]

4-(aminomethyl)benzoic
acid (AMBA)

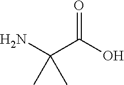

2-methylalanine (AIB) or
2-amino-2-methylpropanoic
acid

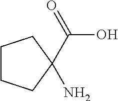

1-aminocyclopentane-1-
carboxylic acid

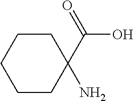

1-aminocyclohexane-1-
carboxylic acid

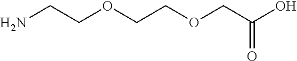 [SMILES: NCCOCCOCC(=O)O]

2-(2-(2-aminoethoxy)ethoxy)
acetic acid

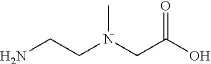

N-(2-aminoethyl)-N-
methylglycine

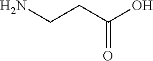

3-aminopropanoic acid

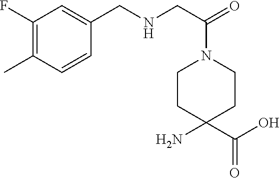 [SMILES: Cc1ccc(CNCC(=O)N2CCC(N)(C(=O)O)CC2)cc1F]

4-amino-1-((3-fluoro-4-
methylbenzyl)glycyl)piperidine-
4-carboxylic acid

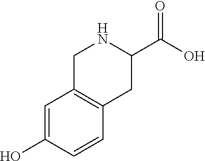 [SMILES: O=C(O)C1Cc2ccc(O)cc2CN1]

7-hydroxy-1,2,3,4-
tetrahydroisoquinoline-3-
carboxylic acid

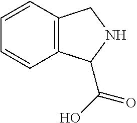 [SMILES: O=C(O)C1NCc2ccccc21]

isoindoline-1-carboxylic
acid

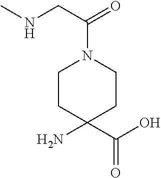

4-amino-1-
(methylglycyl)piperidine-4-
carboxylic acid

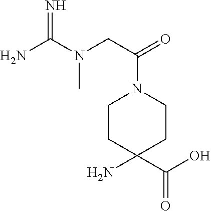 [SMILES: CN(CC(=O)N1CCC(N)(C(=O)O)CC1)C(=N)N]

4-amino-1-(N-
carbamimidoyl-N-
methylglycyl)piperidine-4-
carboxylic acid

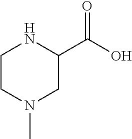

4-methylpiperazine-2-
carboxylic acid

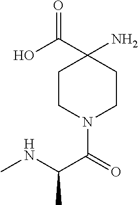 [SMILES: CN[C@H](C)C(=O)N1CCC(N)(C(=O)O)CC1]

4-amino-1-(methyl-D-
alanyl)piperidine-4-carboxylic
acid

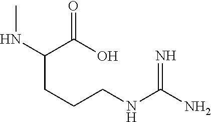

N2-methylarginine

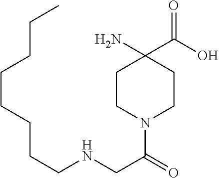 [SMILES: CCCCCCCCNCC(=O)N1CCC(N)(C(=O)O)CC1]

4-amino-1-
(octylglycyl)piperidine-4-
carboxylic acid

TABLE F
Representative β-amino acids

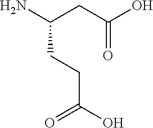 [SMILES: N[C@@H](CCC(=O)O)CC(=O)O]

β³-homoglutamic acid

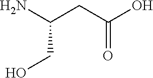

β³-homoserine

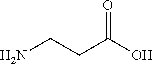

β-alanine (bAla or β-Ala)

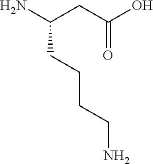 [SMILES: NCCCC[C@H](N)CC(=O)O]

β³-homolysine

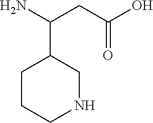 [SMILES: NC(CC(=O)O)C1CCCNC1]

(3-pyrid-3-yl)-β-alanine
(H-βAla(3-pyrid-3-yl)-OH)

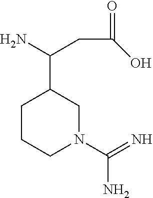 [SMILES: N=C(N)N1CCCC(C(N)CC(=O)O)C1]

3-amino-3-(1-
carbamimidoylpiperidin-3-
yl)propanoic acid

### NUMBERED EMBODIMENTS

Embodiment 1. A compound of Formula (1), or a pharmaceutically acceptable salt thereof:

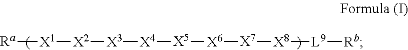

wherein
R^{a} is —C(═O)—R¹⁰,
   R¹⁰ is an aryl, heteroaryl, or partially unsaturated bicyclic heterocyclic ring, each optionally substituted with i to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, or —R¹¹;
   wherein any saturated carbon of R¹⁰ is optionally substituted with oxo (═O);
   R¹¹ is an aryl or heteroaryl optionally substituted with I to 3 substituents that are each independently selected from the group consisting of —F, —Cl, —Br, —C₁-C₃ alkyl, —CF₃, —CN, —OCH₃, —NH₂, or —NO₂;
L⁹ is an optional linker that is attached to any one of X¹, X², X³, X⁴, X⁵, X⁶, X⁷, or X⁸;
R^{b} is a chelating moiety or a radionuclide complex thereof;
X¹ is

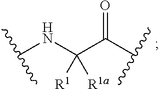

R¹ is -L¹-R^{1b};
   L¹ is absent, —CH₂—, or —CHPh-;
   R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
   wherein each R^{1b} is optionally additionally substituted with 1 or more —F, —Br, —CI, —I, —CF₃, or —C₁-C₆ alkyl;
R^{1a} is hydrogen or methyl;
or R¹ and R^{1a} are taken together with the carbon atoms to which they are attached to form:

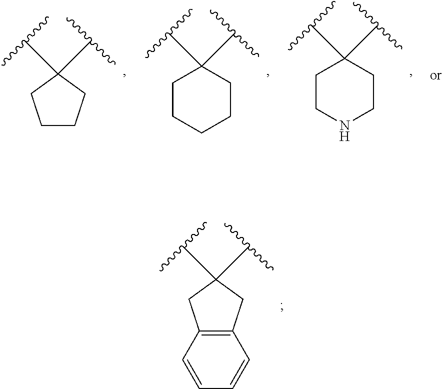 [SMILES: *C1(*)CCCC1 ; *C1(*)CCCCC1 ; *C1(*)CCNCC1 ; *C1(*)Cc2ccccc2C1]

X² is

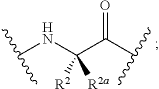

R² is -L²-R^{2b};
   L² is absent, —CH₂—, or —CHPh-;
   R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl; wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

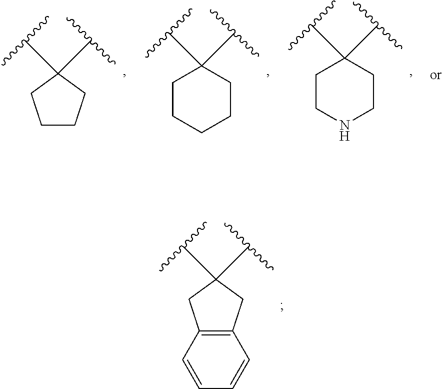 [SMILES: *C1(*)CCCC1 ; *C1(*)CCCCC1 ; *C1(*)CCNCC1 ; *C1(*)Cc2ccccc2C1]

X³ is glycine, (S)-alanine (Ala), (S)-trifluoromethylalanine, (S)-homoalanine, (S)-Glu, (S)-Gln, (S)-Orn, (S)-lysine (Lys), (S)-serine (Ser), (S)—N-methylalanine (N-Me-Ala), 2-amino-2-methylpropanoic acid (AlB), 1-aminocyclopropane-1-carboxylic acid (ACC), norleucine (Nle), (S)-3-aminoalanine, (S)-2-amino-6-azidohexanoic acid, (S)-2-amino-6-(4-hexyl-1H-1,2,3-triazol-1-yl)hexanoic acid, (S)-2-amino-6-(4-((2-methoxyethoxy)methyl)-1H-1,2,3-triazol-l-yl)hexanoic acid, (S)-2-amino-6-(4-(cyclopentylmethyl)-1H-1,2,3-triazol-1-yl)hexanoic acid, (S)-2-amino-6-(4-benzyl-1H-1,2,3-triazol-1-yl)hexanoic acid, (S)-proline (Pro), (S)-cyclopropylglycine, or (S)-biphenylalanine (Bip);
X⁴ is (S)-arginine (Arg), (S)-3-amino-6-guanidinohexanoic acid, N6-carbamimidoyl-(S)-lysine, (S)-2-amino-5-ureidopentanoic acid, (S)-homoarginine, N-methyl-(S)-arginine, N^{ω}-methyl-(S)-arginine, N^{ω}-nitro-(S)-arginine, (E)-N^{ω},N^{ω}′-dimethyl-(S)-arginine, (E)-N^{ω},N^{ω}′-diethyl-(S)-arginine, (S)-ornithine (Orn), (S)-lysine, (S)-phenylalanine (Phe), (S)-cyclohexylglycine (Chg), (S)-cyclohexylalanine (Cha), norleucine (Nle), (S)-3-amino-alanine (H-Dap-OH), piperidinyl-1-yl alanine, N⁴,N⁴-dimethyl-(S)-asparagine (N⁴,N⁴-dimethyl-Asn), (S)-2-(piperidin-4-yl)-glycine (Gly(piperidin-4-yl)), (S)-3-pyridin-3-yl-beta-alanine (H-βAla(3-pyrid-3-yl)-OH), (S)-2-(1-carbamimidoylpiperidin-4-yl)-glycine (Gly(1-carbamimidoylpiperidin-4-yl)), (S)-3-amino-3-(1-carbamimidoylpiperidin-3-yl)propanoic acid, (S)-phenylglycine (Phg), (S)-4-fluorophenylalanine (Phe(4-F)), (S)-4-aminophenylalanine (Phe(4-NH₂)), (S)-4-nitrophenylalanine (Phe(4-NO₂)), (S)-4-carbamoylphenylalanine, (S)-4-guanidinophenylalanine, (S)-asparagine (Asn), (S)-glutamic acid (Glu), (S)-proline, (S)-4-ureidophenylalanine, (S)-(2-aminoethoxy)phenylalanine (Phe(4-OCH₂CH₂NH₂)), (S)-(pyridin-4-yl)alanine (3-Pal), (S)-(pyridin-4-yl)alanine (4-Pal), (S)-3-(6-aminopyridin-3-yl)alanine ((3-Pal(6-NH₂)), (S)-2-((2-aminopyridin-4-yl)-λ²-azaneyl)ethyl alanine, N6-nicotinoyl-Lys, 2-oxo-2-(piperazin-1-yl)ethyl alanine, (S)-2-(piperidin-4-yl)ethylalanine, (S)-3-((piperazin-1-yl)propyl)-glycine, (S)-3-((4-methylpiperazin-1-yl)propyl)-glycine, (S)-3-(4-benzylpiperidin-1-yl)propylglycine, (S)-3-((4-benzylpiperazin-1-yl)propyl)-glycine, N6-(1-benzylpiperidine-4-carbonyl)-Lys, (S)-3-((3,4-dihydroisoquinolin-2(1H)-yl)propyl)-glycine, (S)-2-pyridin-4-ylthynyl alanine, (S)-(4-(aminomethyl)-1H-1,2λ²,3λ²-triazol-1-yl)methyl)-glycine, (S)-(4-(piperazin-1-ylmethyl)-1H-1,2λ²,3λ²-triazol-1-yl)methyl)-glycine, (S)-2-((1H-imidazol-4-yl)ethyl)glycine, or (4-((R)-2,6-dioxohexahydropyrimidine-4-carboxamido)phenyl)alanine;
or X⁴ is (S)-4-methylpiperazine-2-carboxylic acid,
or X⁴ is

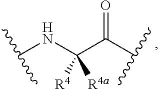

where R⁴ and R^{4a} are taken together with the carbon atom to which they are attached to form

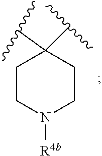

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

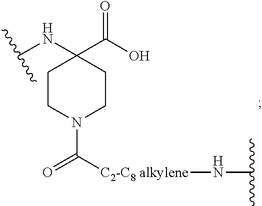

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Ly s, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-J)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ2-triazol-1-yl)methyl)-glycine;
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

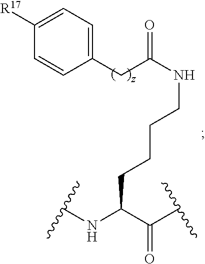

R¹⁷ is —CH₃ or —I;
z is 1, 2, or 3; and
X⁸ is absent, lysine (Lys), or glutamic acid (Glu).
L⁵, L⁶, and L⁷ are each independently —NR¹⁵-L¹³-L¹⁴-C(═O)— or substituted or unsubstituted piperazinylene-;
   L¹³ is absent, —C₁-C₆ alkylene, substituted or unsubstituted heterocycloalkylene, substituted or unsubstituted cyclohexylene, —(CH₂CH₂O)_{y}—, or —(CH₂CH₂NR¹⁶)_{y}—;
   L¹⁴ is absent, —C₁-C₆ alkylene, substituted or unsubstituted heterocycloalkylene, substituted or unsubstituted cyclohexylene;
   R¹⁵ and R¹⁶ are each independently selected from H or —C₁-C₄ alkylene; and
y is 1, 2, 3, 4, 5, or 6;
wherein the compound of Formula (I) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (I) may be either —COOH or —CONH₂.

Embodiment 2. The compound of embodiment 1, or a pharmaceutically acceptable salt thereof, wherein
R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, or —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, or —NO₂;
L⁹ is an optional linker that is attached to any one of X⁵, X⁶, or X⁷;
R^{b} is a chelating moiety or a radionuclide complex thereof;
X¹ is

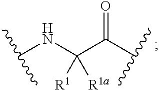

R¹ is -L¹-R^{1b};
   L¹ is absent or —CH₂—;
   R^{1b} is cyclopentyl, cyclohexyl, cycloheptyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, 2-chlorophenyl, 3-chlorophenyl, 4-chlorophenyl, 2,4-dichlorophenyl, 3,4-dichlorophenyl, or 2,5-dichlorophenyl;
R^{1a} is hydrogen or methyl;
X² is

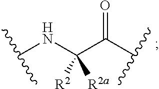

R² is -L²-R^{2b};
   L² is absent or —CH₂;
   R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl, or 4′-methyl-[1,1′-biphenyl]-4-yl;
R^{2a} is hydrogen or methyl;
X³ is glycine, (S)-alanine (Ala), or norleucine (Nle);
X⁴ is (S)-arginine (Arg), (S)-ornithine (Orn), (S)-phenylalanine (Phe), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-(pyridin-4-yl)alanine (4-Pal), (S)-4-aminophenylalanine (Phe(4-NH₂)), (S)-3-(6-aminopyridin-3-yl)alanine ((3-Pal(6-NH₂)), or (S)-2-((2-aminopyridin-4-yl)-λ²-azaneyl)ethyl alanine;
X⁵ is alanine (Ala), lysine (Lys), ornithine (Orn),

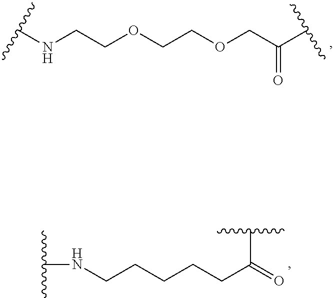 [SMILES: *NCCOCCOCC(*)=O ; *NCCCCCC(*)=O]

N6-(4-fluorobenzoyl)-Lys, β³-homolysine, 3-amino-alanine (H-Dap-OH), 4-nitrophenylalanine (Phe(4-NO₂)), or (4-fluorobenzamido)methylglycine;
X⁶ is absent, lysine (Lys), ornithine (Orn),

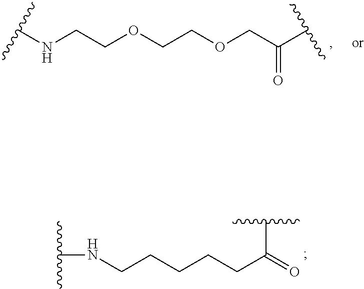 [SMILES: *NCCOCCOCC(*)=O ; *NCCCCCC(*)=O]

X⁷ is absent, lysine (Lys), ornithine (Orn), or

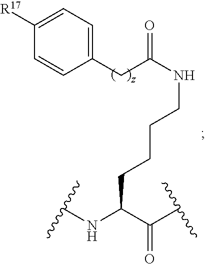

R¹⁷ is —CH₃ or —I;
z is 1, 2, or 3; and
X⁸ is absent.

Embodiment 3. The compound of embodiment I or 2, or a pharmaceutically acceptable salt thereof, wherein the compound has the structure of Formula (Ia):

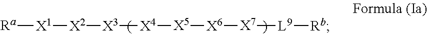

wherein L⁹ is an optional linker that is attached to any one of X⁴, X⁵, X⁶, or X⁷.

Embodiment 4. The compound of embodiment 1 or 2, or a pharmaceutically acceptable salt thereof, wherein the compound has the structure of Formula (Ib):

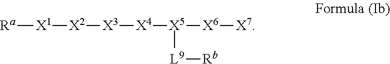

Embodiment 5. The compound of embodiment 4, or a pharmaceutically acceptable salt thereof, wherein X⁶ and X⁷ are absent.

Embodiment 6. The compound of embodiment 4, or a pharmaceutically acceptable salt thereof, wherein the compound of Formula (Ib) has the following structure:

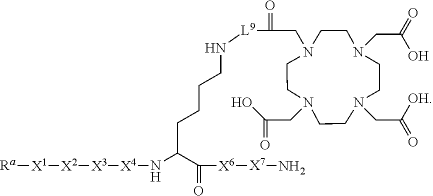

Embodiment 7. The compound of embodiment 4, or a pharmaceutically acceptable salt thereof, wherein the compound of Formula (Ib) has the following structure:

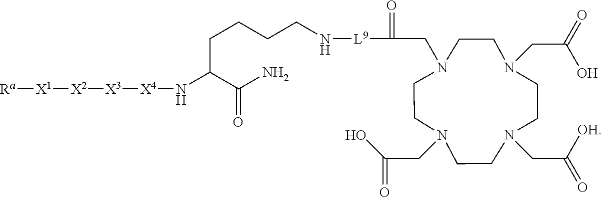

Embodiment 8. The compound of embodiment 4, or a pharmaceutically acceptable salt thereof, wherein the compound of Formula (Ib) has the following structure:

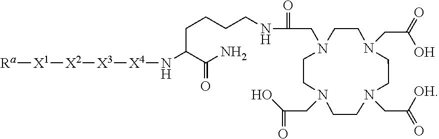

Embodiment 9. The compound of embodiment 4, or a pharmaceutically acceptable salt thereof, wherein the compound of Formula (Ib) has the following structure:

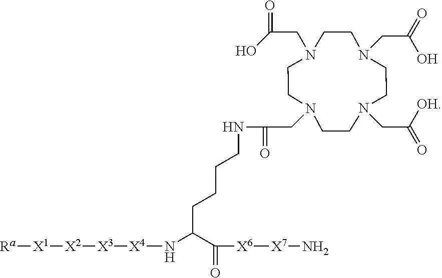

Embodiment 10. The compound of embodiment 4, or a pharmaceutically acceptable salt thereof, wherein the compound of Formula (Ib) has the following structure:

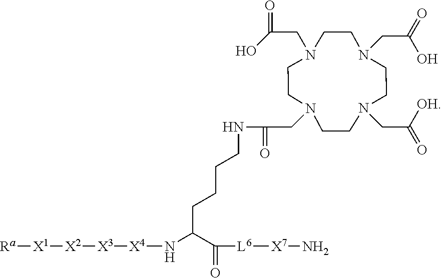

Embodiment 11. The compound of embodiment 4, or a pharmaceutically acceptable salt thereof, wherein the compound of Formula (Ib) has one of the following structures:

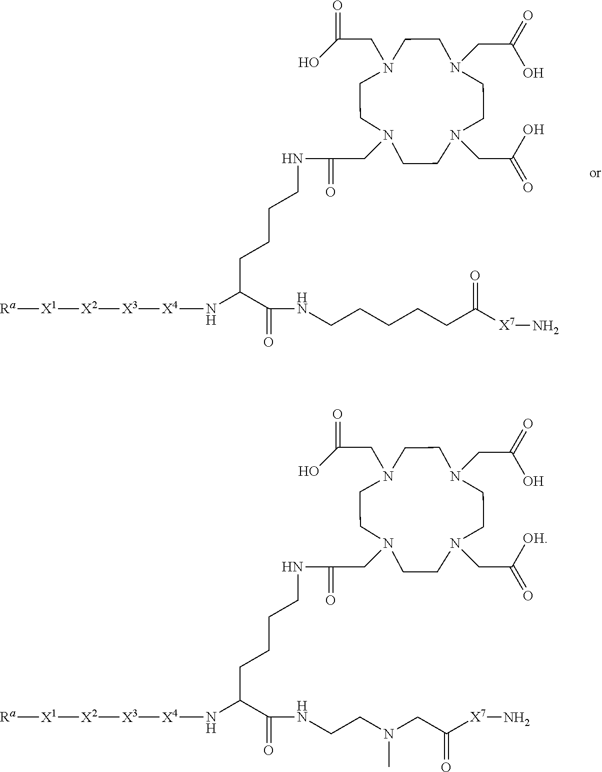

Embodiment 12. The compound of embodiment 4, or a pharmaceutically acceptable salt thereof, wherein the compound of Formula (Ib) has the following structure

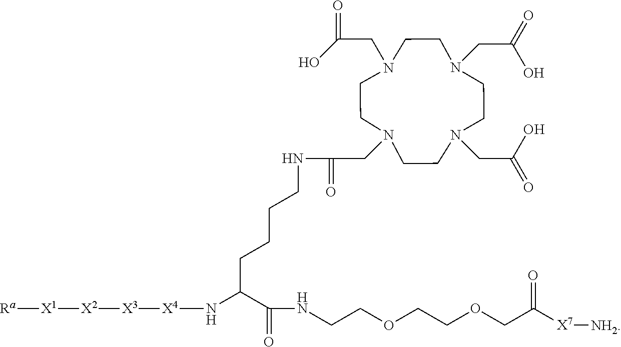

Embodiment 13. The compound of embodiment 1 or 2, or a pharmaceutically acceptable salt thereof, wherein the compound has the structure of Formula (Ic):

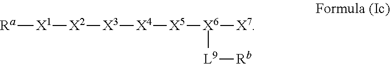

Embodiment 14. The compound of embodiment 13, or a pharmaceutically acceptable salt thereof, wherein the compound of Formula (Ic) has the following structure:

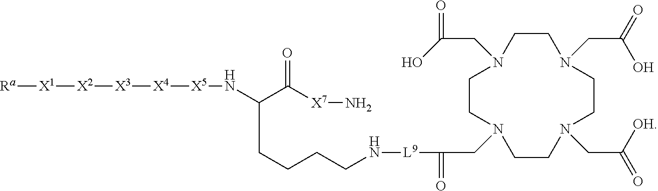

Embodiment 15. The compound of embodiment 13, or a pharmaceutically acceptable salt thereof, wherein the compound of Formula (Ic) has the following structure:

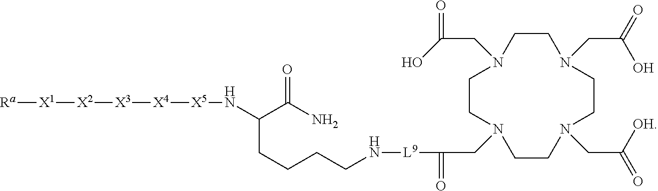

Embodiment 16. The compound of embodiment 13, or a pharmaceutically acceptable salt thereof, wherein the compound of Formula (Ic) has the following structure:

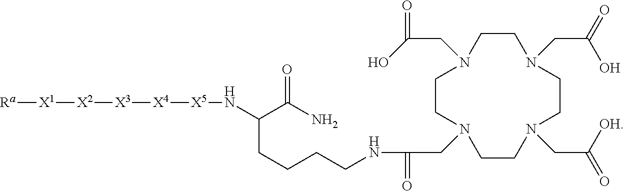

Embodiment 17. The compound of embodiment 13, or a pharmaceutically acceptable salt thereof, wherein the compound of Formula (Ic) has the following structure:

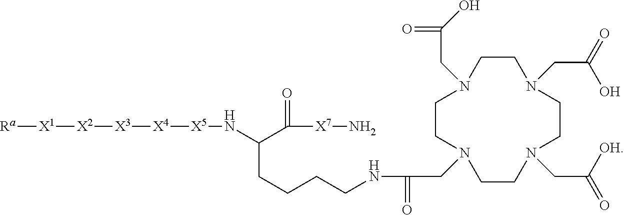

Embodiment 18. The compound of embodiment 1 or 2, or a pharmaceutically acceptable salt thereof, wherein the compound has the structure of Formula (Id):

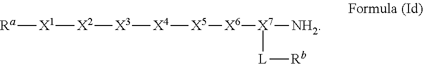

Embodiment 19. The compound of embodiment 18, wherein the compound of Formula (Id) has the following structure:

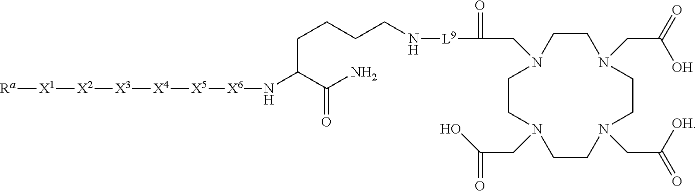

Embodiment 20. The compound of embodiment 18, or a pharmaceutically acceptable salt thereof, wherein the compound of Formula (Id) has the following structure:

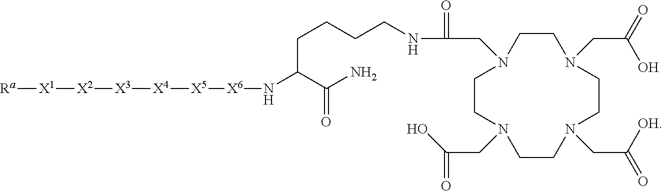

Embodiment 21. The compound of any one of embodiments 1-6, 13-15, 18 or 19, or a pharmaceutically acceptable salt thereof, wherein L⁹ is a bond.

Embodiment 22. The compound of any one of embodiments 1-21, or a pharmaceutically acceptable salt thereof, wherein X¹ is

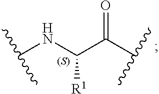

R¹ is -L¹-R^{1b};
L¹ is —CH₂—; and
R^{1b} is cyclopentyl, cyclohexyl, cycloheptyl, cyclooctyl, phenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, pyridin-4-yl, 2,3-dihydro-1-inden-2-yl, 3-cyclopropylphenyl, or [1,1′-biphenyl]-4-yl.

Embodiment 23. The compound of any one of embodiments 1-22, or a pharmaceutically acceptable salt thereof, wherein R^{1b} is cyclohexyl or cycloheptyl.

Embodiment 24. The compound of any one of embodiments 1-22, or a pharmaceutically acceptable salt thereof, wherein R^{1b} is phenyl, 4-fluorophenyl, 2,4-difluorophenyl, 3,4-difluorophenyl, 2,5-difluorophenyl, 3,4,5-trifluorophenyl, or perfluorophenyl.

Embodiment 25. The compound of any one of embodiments 1-24, or a pharmaceutically acceptable salt thereof, wherein X² is

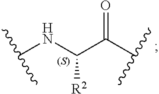

R² is -L²-R^{2b};
L² is absent; and
R^{2b} is cyclohexyl or cycloheptyl.

Embodiment 26. The compound of any one of embodiments 1-24, or a pharmaceutically acceptable salt thereof, wherein X² is

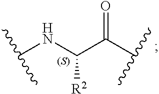

R² is -L²-R^{2b};
L² is absent; and
R^{2b} is 2,3-dihydro-1H-inden-2-yl or [1,1′-biphenyl]-4-yl.

Embodiment 27. The compound of any one of embodiments 1-26, or a pharmaceutically acceptable salt thereof, wherein X³ is (S)-alanine (Ala) or (S)-norleucine (Nle).

Embodiment 28. The compound of any one of embodiments 1-26, or a pharmaceutically acceptable salt thereof, wherein X³ is (S)-alanine (Ala).

Embodiment 29. The compound of any one of embodiments 1-28, or a pharmaceutically acceptable salt thereof, wherein X⁴ is (S)-arginine (Arg), (S)-ornithine (Orn), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-3-(6-aminopyridin-3-yl)alanine ((3-Pal(6-NH₂)), or (S)-2-((2-aminopyridin-4-yl)-λ²-azaneyl)ethyl alanine.

Embodiment 30. The compound of any one of embodiments 1-28, or a pharmaceutically acceptable salt thereof, wherein X⁴ is (S)-arginine (Arg).

Embodiment 31. The compound of any one of embodiments 1-28, or a pharmaceutically acceptable salt thereof, wherein X⁴ is (S)-2-(1-carbamimidoylpiperidin-4-yl)-glycine (Gly(1-carbamimidoylpiperidin-4-yl)).

Embodiment 32. The compound of any one of embodiments 1-28, or a pharmaceutically acceptable salt thereof, wherein X⁴ is (S)-2-amino-3-(4-aminophenyl)propanoic acid (Phe(4-NH₂)), (S)-2-amino-3-(6-aminopyridin-3-yl)propanoic acid, or (S)-2-((2-aminopyridin-4-yl)-λ²-azaneyl)ethyl alanine.

Embodiment 33. The compound of any one of embodiments 1-28, or a pharmaceutically acceptable salt thereof, wherein X⁴ is

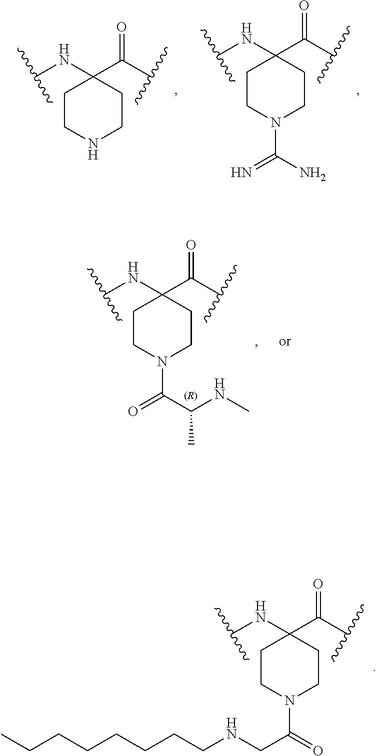

Embodiment 34. The compound of any one of embodiments 1-28, or a pharmaceutically acceptable salt thereof, wherein X⁴ is

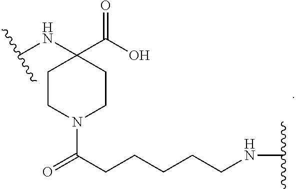 [SMILES: *NCCCCCC(=O)N1CCC(N*)(C(=O)O)CC1]

Embodiment 35. The compound of any one of embodiments 1-21, wherein —X¹—X²—X³—X⁴— is:

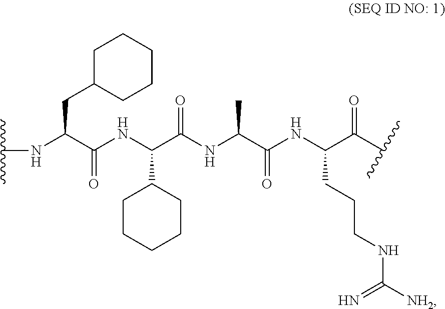 [SMILES: *N[C@@H](CC1CCCCC1)C(=O)N[C@H](C(=O)N[C@@H](C)C(=O)N[C@@H](CCCNC(=N)N)C(*)=O)C1CCCCC1]

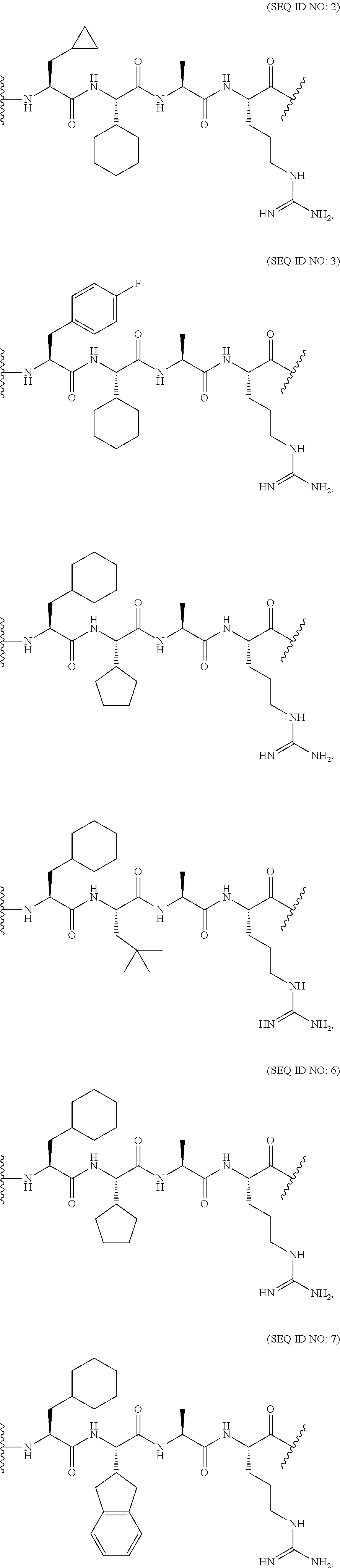

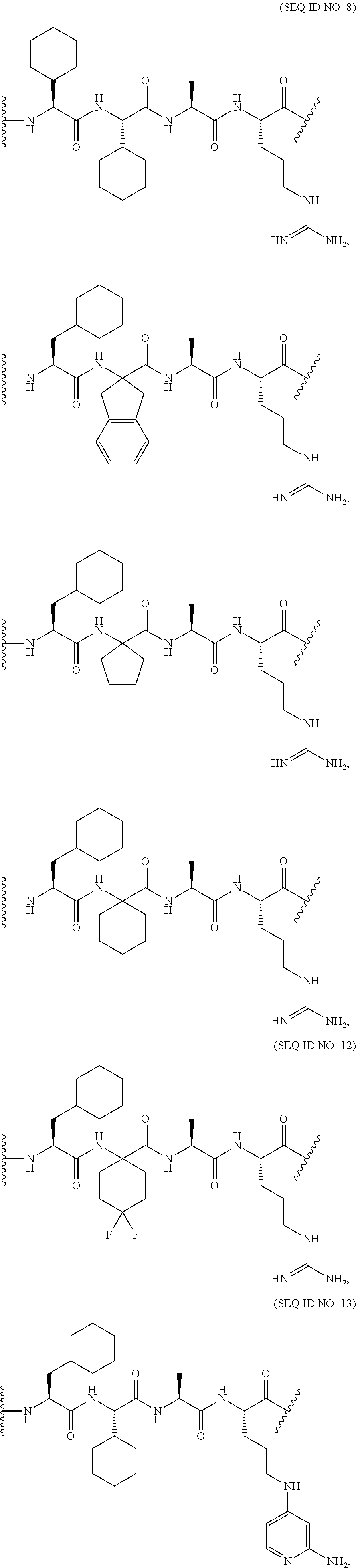

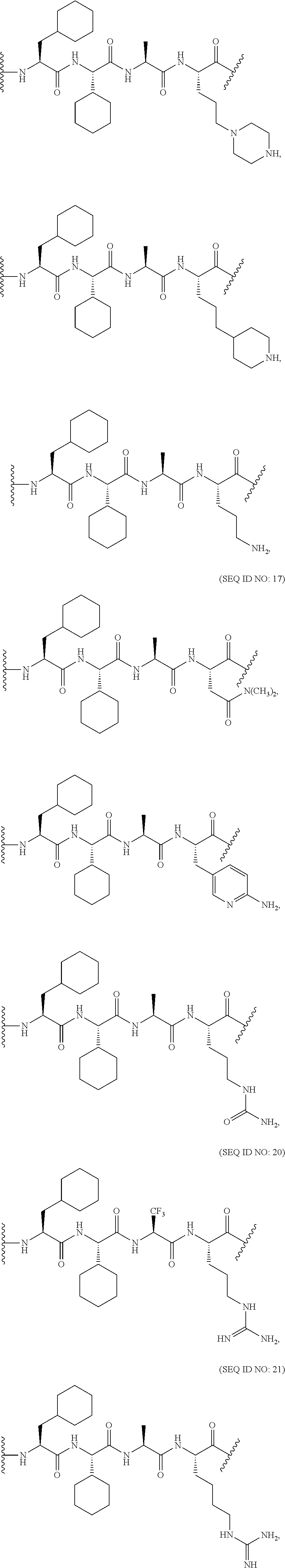

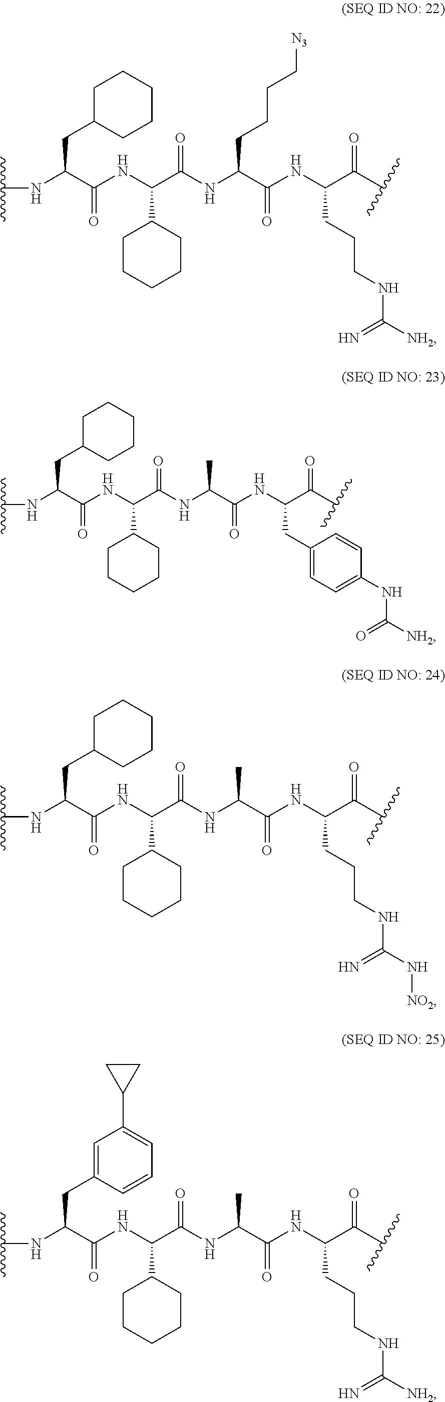 [SMILES: *N[C@@H](CC1CCCCC1)C(=O)N[C@H](C(=O)N[C@@H](CCCCN=[N+]=[N-])C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N[C@@H](Cc1ccc(NC(N)=O)cc1)C(*)=O)C1CCCCC1 ; *N[C@@H](CC1CCCCC1)C(=O)N[C@H](C(=O)N[C@@H](C)C(=O)N[C@@H](CCCNC(=N)N[N+](=O)[O-])C(*)=O)C1CCCCC1 ; *N[C@@H](Cc1cccc(C2CC2)c1)C(=O)N[C@H](C(=O)N[C@@H](C)C(=O)N[C@@H](CCCNC(=N)N)C(*)=O)C1CCCCC1]

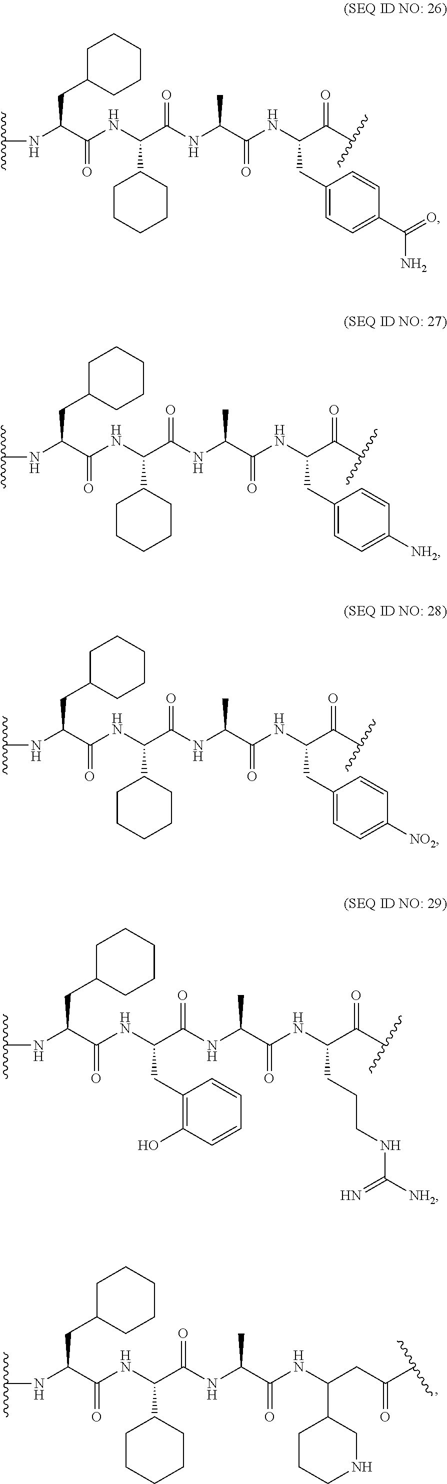 [SMILES: *N[C@@H](CC1CCCCC1)C(=O)N[C@H](C(=O)N[C@@H](C)C(=O)N[C@@H](Cc1ccc(C(N)=O)cc1)C(*)=O)C1CCCCC1 ; *N[C@@H](CC1CCCCC1)C(=O)N[C@H](C(=O)N[C@@H](C)C(=O)N[C@@H](Cc1ccc(N)cc1)C(*)=O)C1CCCCC1 ; *N[C@@H](CC1CCCCC1)C(=O)N[C@H](C(=O)N[C@@H](C)C(=O)N[C@@H](Cc1ccc([N+](=O)[O-])cc1)C(*)=O)C1CCCCC1 ; *N[C@@H](CC1CCCCC1)C(=O)N[C@@H](Cc1ccccc1O)C(=O)N[C@@H](C)C(=O)N[C@@H](CCCNC(=N)N)C(*)=O ; *N[C@@H](CC1CCCCC1)C(=O)N[C@H](C(=O)N[C@@H](C)C(=O)NC(CC(*)=O)C1CCCNC1)C1CCCCC1]

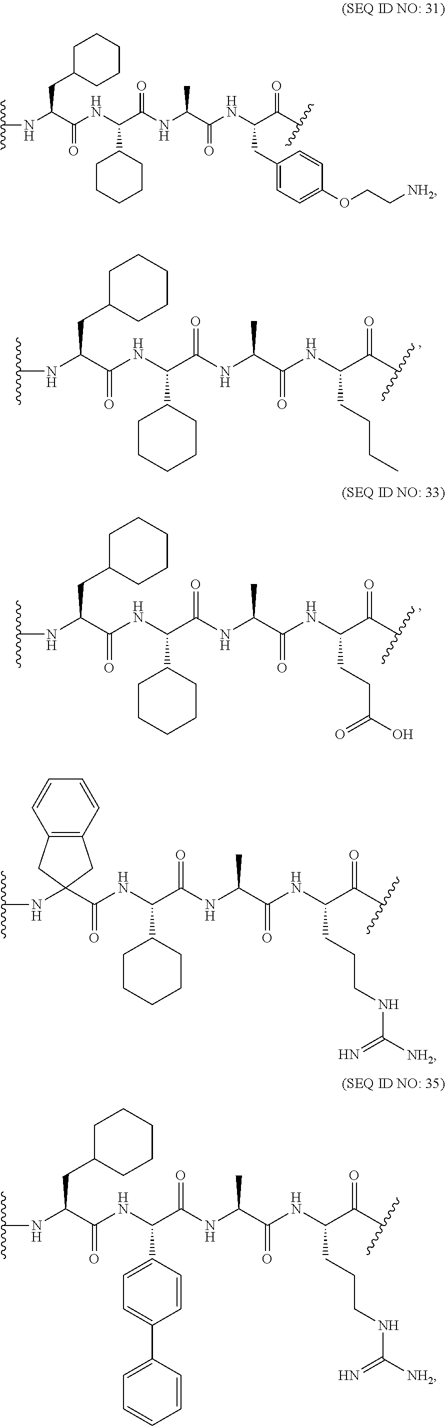 [SMILES: *N[C@@H](CC1CCCCC1)C(=O)N[C@H](C(=O)N[C@@H](C)C(=O)N[C@@H](Cc1ccc(OCCN)cc1)C(*)=O)C1CCCCC1 ; *N[C@@H](CC1CCCCC1)C(=O)N[C@H](C(=O)N[C@@H](C)C(=O)N[C@@H](CCCC)C(*)=O)C1CCCCC1 ; *N[C@@H](CC1CCCCC1)C(=O)N[C@H](C(=O)N[C@@H](C)C(=O)N[C@@H](CCC(=O)O)C(*)=O)C1CCCCC1 ; *NC1(C(=O)N[C@H](C(=O)N[C@@H](C)C(=O)N[C@@H](CCCNC(=N)N)C(*)=O)C2CCCCC2)Cc2ccccc2C1 ; *N[C@@H](CC1CCCCC1)C(=O)N[C@H](C(=O)N[C@@H](C)C(=O)N[C@@H](CCCNC(=N)N)C(*)=O)c1ccc(-c2ccccc2)cc1]

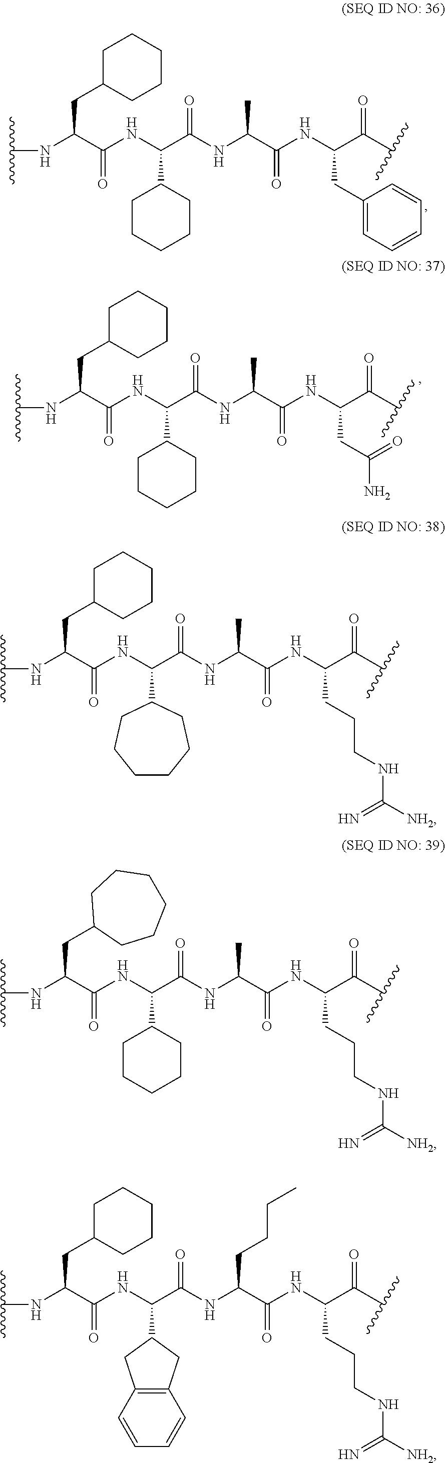

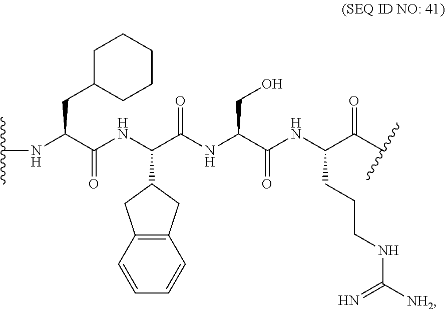 [SMILES: *N[C@@H](CC1CCCCC1)C(=O)N[C@H](C(=O)N[C@@H](CO)C(=O)N[C@@H](CCCNC(=N)N)C(*)=O)C1Cc2ccccc2C1]

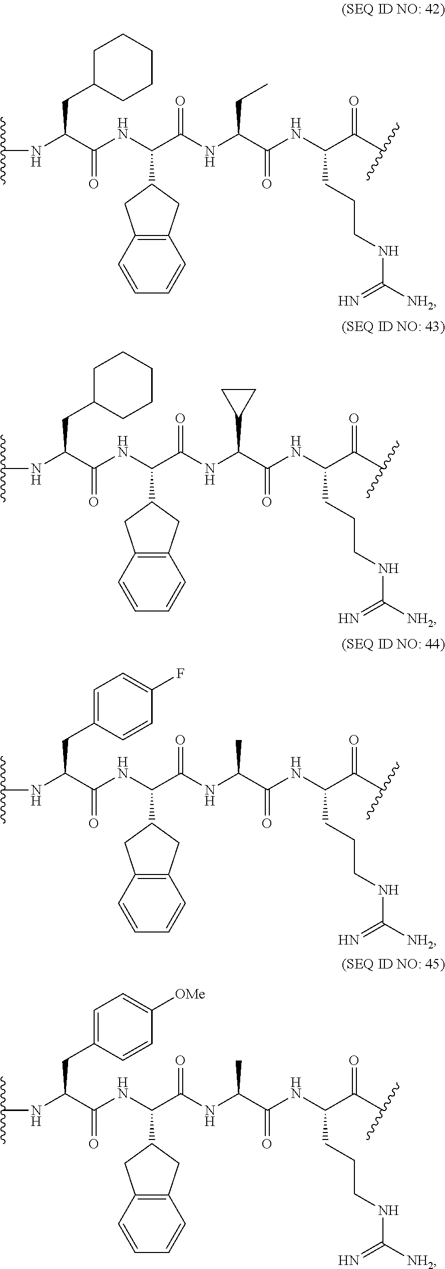 [SMILES: *N[C@@H](CC1CCCCC1)C(=O)N[C@H](C(=O)N[C@@H](CC)C(=O)N[C@@H](CCCNC(=N)N)C(*)=O)C1Cc2ccccc2C1 ; *N[C@@H](CC1CCCCC1)C(=O)N[C@H](C(=O)N[C@H](C(=O)N[C@@H](CCCNC(=N)N)C(*)=O)C1CC1)C1Cc2ccccc2C1 ; *N[C@@H](Cc1ccc(F)cc1)C(=O)N[C@H](C(=O)N[C@@H](C)C(=O)N[C@@H](CCCNC(=N)N)C(*)=O)C1Cc2ccccc2C1 ; *N[C@@H](Cc1ccc(OC)cc1)C(=O)N[C@H](C(=O)N[C@@H](C)C(=O)N[C@@H](CCCNC(=N)N)C(*)=O)C1Cc2ccccc2C1]

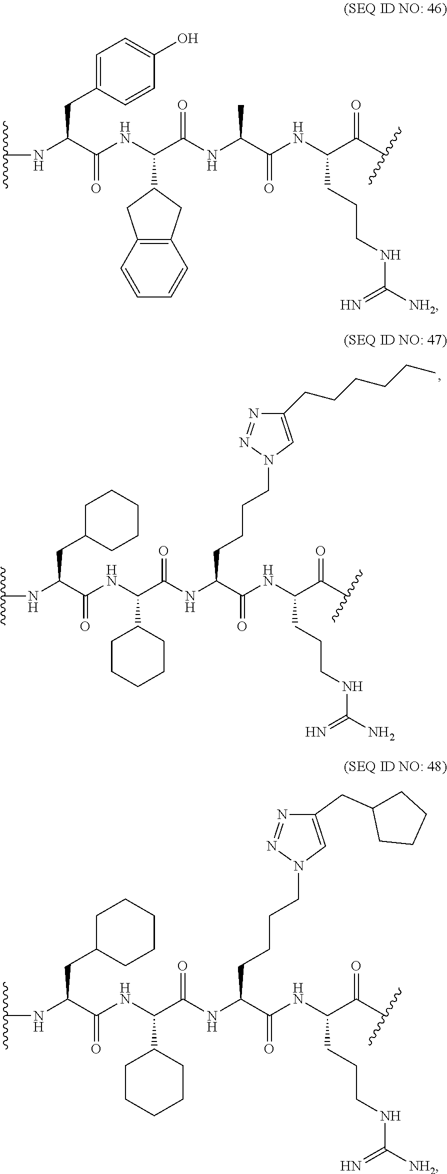 [SMILES: *N[C@@H](Cc1ccc(O)cc1)C(=O)N[C@H](C(=O)N[C@@H](C)C(=O)N[C@@H](CCCNC(=N)N)C(*)=O)C1Cc2ccccc2C1 ; *N[C@@H](CC1CCCCC1)C(=O)N[C@H](C(=O)N[C@@H](CCCCn1cc(CCCCCC)nn1)C(=O)N[C@@H](CCCNC(=N)N)C(*)=O)C1CCCCC1 ; *N[C@@H](CC1CCCCC1)C(=O)N[C@H](C(=O)N[C@@H](CCCCn1cc(CC2CCCC2)nn1)C(=O)N[C@@H](CCCNC(=N)N)C(*)=O)C1CCCCC1]

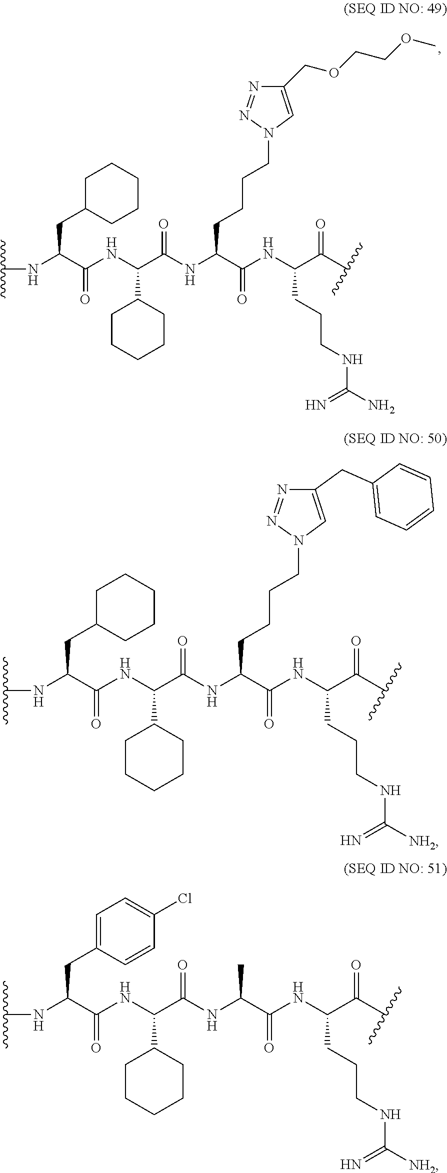 [SMILES: *N[C@@H](CC1CCCCC1)C(=O)N[C@H](C(=O)N[C@@H](CCCCn1cc(COCCOC)nn1)C(=O)N[C@@H](CCCNC(=N)N)C(*)=O)C1CCCCC1 ; *N[C@@H](CC1CCCCC1)C(=O)N[C@H](C(=O)N[C@@H](CCCCn1cc(Cc2ccccc2)nn1)C(=O)N[C@@H](CCCNC(=N)N)C(*)=O)C1CCCCC1 ; *N[C@@H](Cc1ccc(Cl)cc1)C(=O)N[C@H](C(=O)N[C@@H](C)C(=O)N[C@@H](CCCNC(=N)N)C(*)=O)C1CCCCC1]

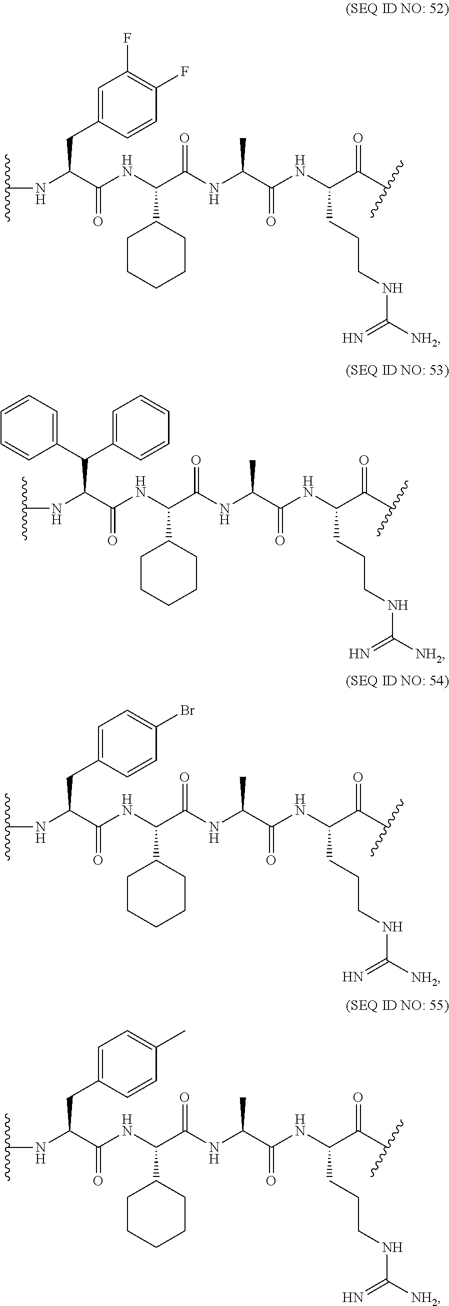 [SMILES: *N[C@@H](Cc1ccc(F)c(F)c1)C(=O)N[C@H](C(=O)N[C@@H](C)C(=O)N[C@@H](CCCNC(=N)N)C(*)=O)C1CCCCC1 ; *N[C@H](C(=O)N[C@H](C(=O)N[C@@H](C)C(=O)N[C@@H](CCCNC(=N)N)C(*)=O)C1CCCCC1)C(c1ccccc1)c1ccccc1 ; *N[C@@H](Cc1ccc(Br)cc1)C(=O)N[C@H](C(=O)N[C@@H](C)C(=O)N[C@@H](CCCNC(=N)N)C(*)=O)C1CCCCC1 ; *N[C@@H](Cc1ccc(C)cc1)C(=O)N[C@H](C(=O)N[C@@H](C)C(=O)N[C@@H](CCCNC(=N)N)C(*)=O)C1CCCCC1]

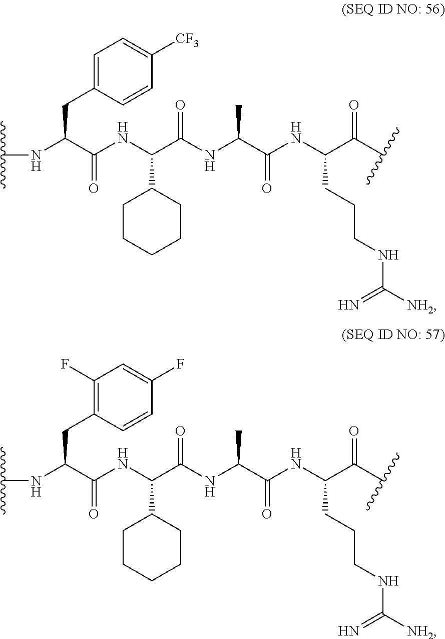 [SMILES: *N[C@@H](Cc1ccc(C(F)(F)F)cc1)C(=O)N[C@H](C(=O)N[C@@H](C)C(=O)N[C@@H](CCCNC(=N)N)C(*)=O)C1CCCCC1 ; *N[C@@H](Cc1ccc(F)cc1F)C(=O)N[C@H](C(=O)N[C@@H](C)C(=O)N[C@@H](CCCNC(=N)N)C(*)=O)C1CCCCC1]

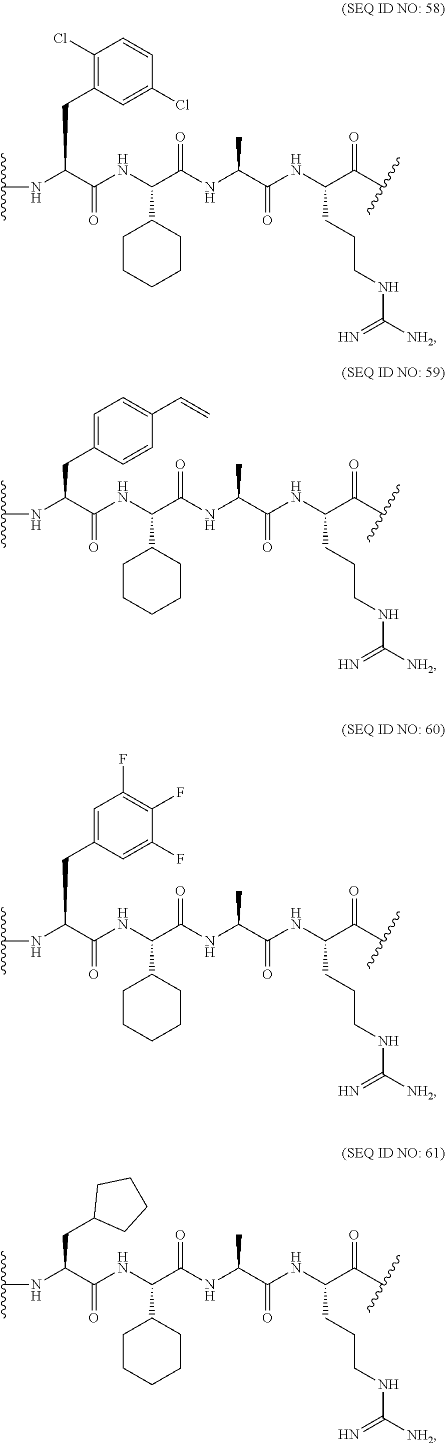 [SMILES: *N[C@@H](Cc1cc(Cl)ccc1Cl)C(=O)N[C@H](C(=O)N[C@@H](C)C(=O)N[C@@H](CCCNC(=N)N)C(*)=O)C1CCCCC1 ; *N[C@@H](Cc1ccc(C=C)cc1)C(=O)N[C@H](C(=O)N[C@@H](C)C(=O)N[C@@H](CCCNC(=N)N)C(*)=O)C1CCCCC1 ; *N[C@@H](Cc1cc(F)c(F)c(F)c1)C(=O)N[C@H](C(=O)N[C@@H](C)C(=O)N[C@@H](CCCNC(=N)N)C(*)=O)C1CCCCC1 ; *N[C@@H](CC1CCCC1)C(=O)N[C@H](C(=O)N[C@@H](C)C(=O)N[C@@H](CCCNC(=N)N)C(*)=O)C1CCCCC1]

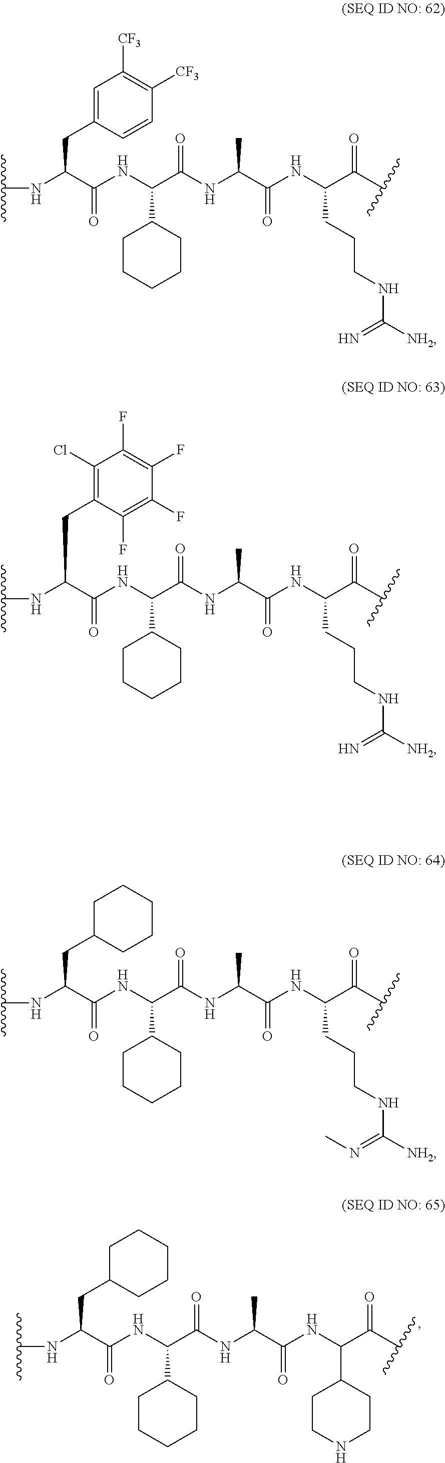 [SMILES: *N[C@@H](Cc1ccc(C(F)(F)F)c(C(F)(F)F)c1)C(=O)N[C@H](C(=O)N[C@@H](C)C(=O)N[C@@H](CCCNC(=N)N)C(*)=O)C1CCCCC1 ; *N[C@@H](Cc1c(F)c(F)c(F)c(F)c1Cl)C(=O)N[C@H](C(=O)N[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N[C@@H](CCCN/C(N)=N\C)C(*)=O)C1CCCCC1 ; *N[C@@H](CC1CCCCC1)C(=O)N[C@H](C(=O)N[C@@H](C)C(=O)NC(C(*)=O)C1CCNCC1)C1CCCCC1]

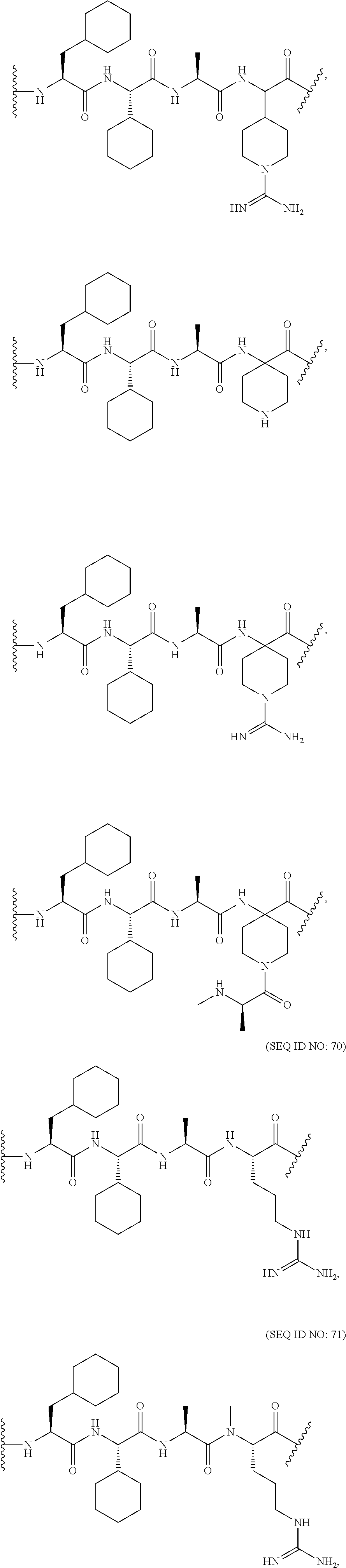

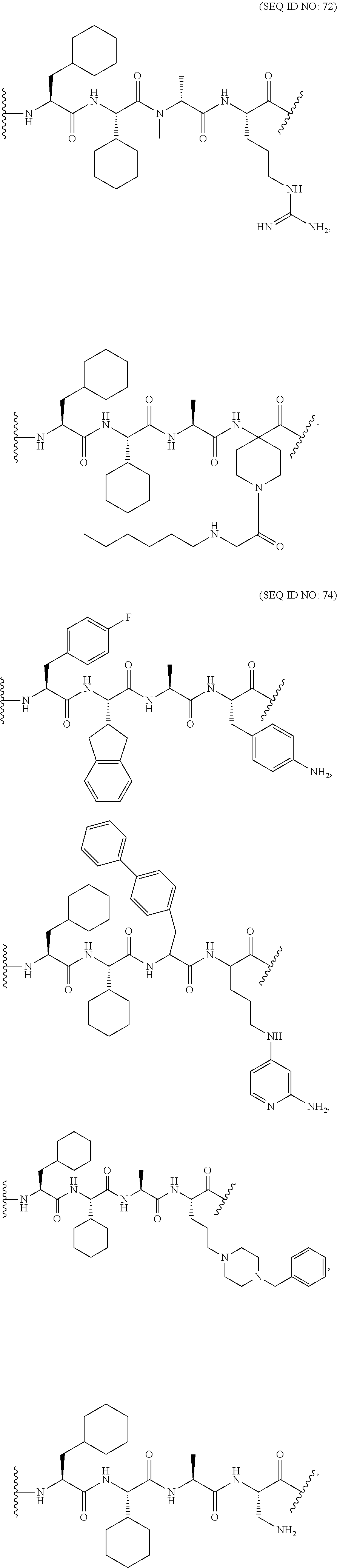

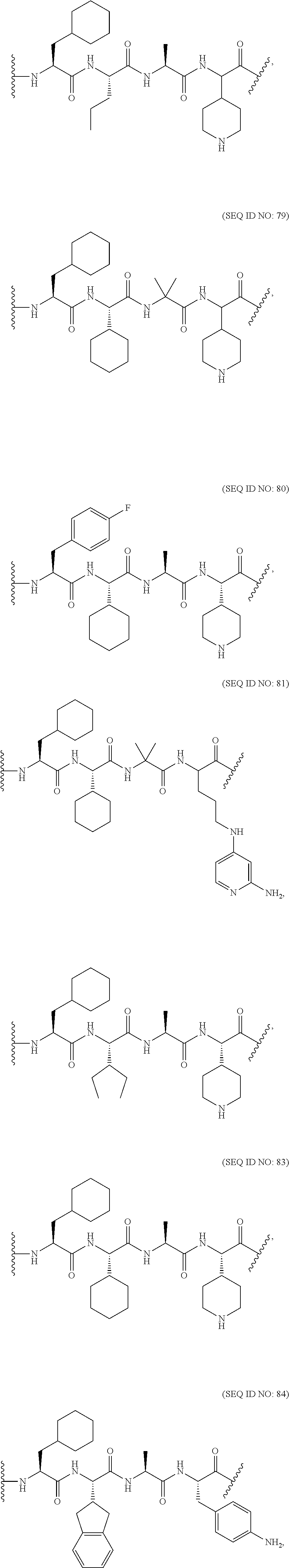

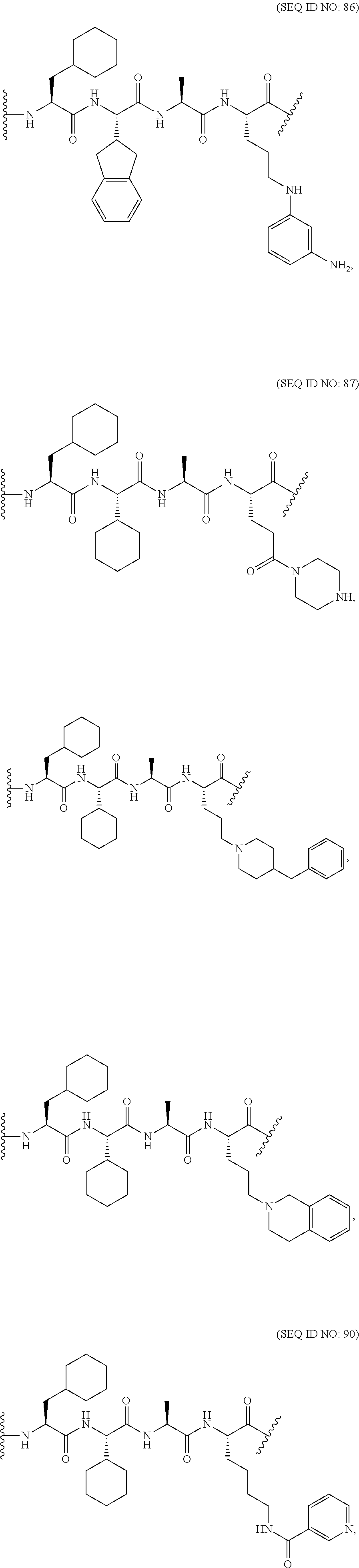 [SMILES: *N[C@@H](CC1CCCCC1)C(=O)N[C@H](C(=O)N[C@@H](C)C(=O)N[C@@H](CCCNc1cccc(N)c1)C(*)=O)C1Cc2ccccc2C1 ; *N[C@@H](CC1CCCCC1)C(=O)N[C@H](C(=O)N[C@@H](C)C(=O)N[C@@H](CCC(=O)N1CCNCC1)C(*)=O)C1CCCCC1 ; *N[C@@H](CC1CCCCC1)C(=O)N[C@H](C(=O)N[C@@H](C)C(=O)N[C@@H](CCCN1CCC(Cc2ccccc2)CC1)C(*)=O)C1CCCCC1 ; *N[C@@H](CC1CCCCC1)C(=O)N[C@H](C(=O)N[C@@H](C)C(=O)N[C@@H](CCCN1CCc2ccccc2C1)C(*)=O)C1CCCCC1 ; *N[C@@H](CC1CCCCC1)C(=O)N[C@H](C(=O)N[C@@H](C)C(=O)N[C@@H](CCCCNC(=O)c1cccnc1)C(*)=O)C1CCCCC1]

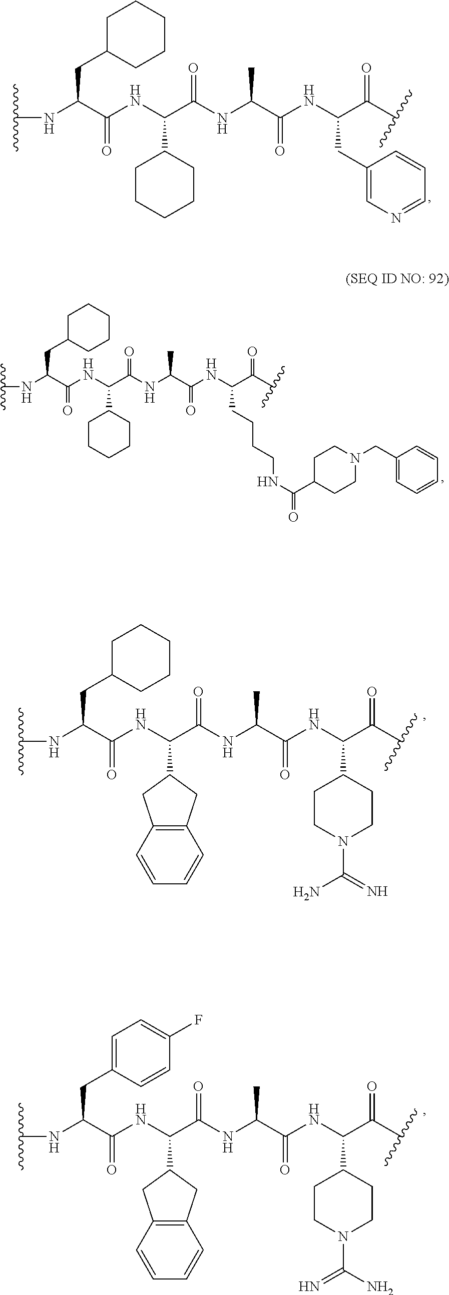 [SMILES: *N[C@@H](CC1CCCCC1)C(=O)N[C@H](C(=O)N[C@@H](C)C(=O)N[C@@H](Cc1cccnc1)C(*)=O)C1CCCCC1 ; *N[C@@H](CC1CCCCC1)C(=O)N[C@H](C(=O)N[C@@H](C)C(=O)N[C@@H](CCCCNC(=O)C1CCN(Cc2ccccc2)CC1)C(*)=O)C1CCCCC1 ; *N[C@@H](CC1CCCCC1)C(=O)N[C@H](C(=O)N[C@@H](C)C(=O)N[C@H](C(*)=O)C1CCN(C(=N)N)CC1)C1Cc2ccccc2C1 ; *N[C@@H](Cc1ccc(F)cc1)C(=O)N[C@H](C(=O)N[C@@H](C)C(=O)N[C@H](C(*)=O)C1CCN(C(=N)N)CC1)C1Cc2ccccc2C1]

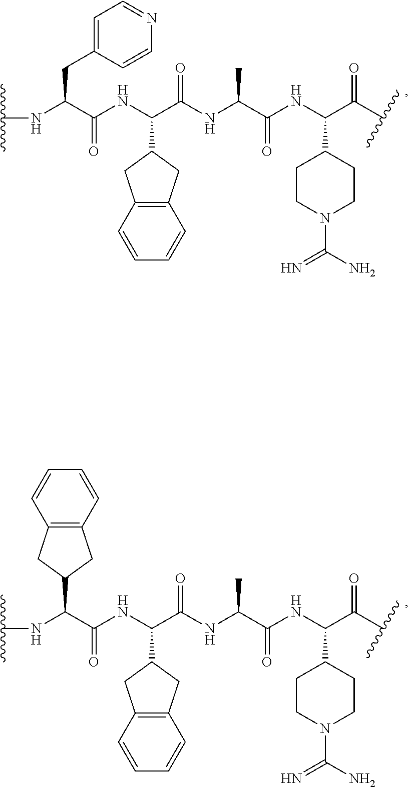 [SMILES: *N[C@@H](Cc1ccncc1)C(=O)N[C@H](C(=O)N[C@@H](C)C(=O)N[C@H](C(*)=O)C1CCN(C(=N)N)CC1)C1Cc2ccccc2C1 ; *N[C@H](C(=O)N[C@H](C(=O)N[C@@H](C)C(=O)N[C@H](C(*)=O)C1CCN(C(=N)N)CC1)C1Cc2ccccc2C1)C1Cc2ccccc2C1]

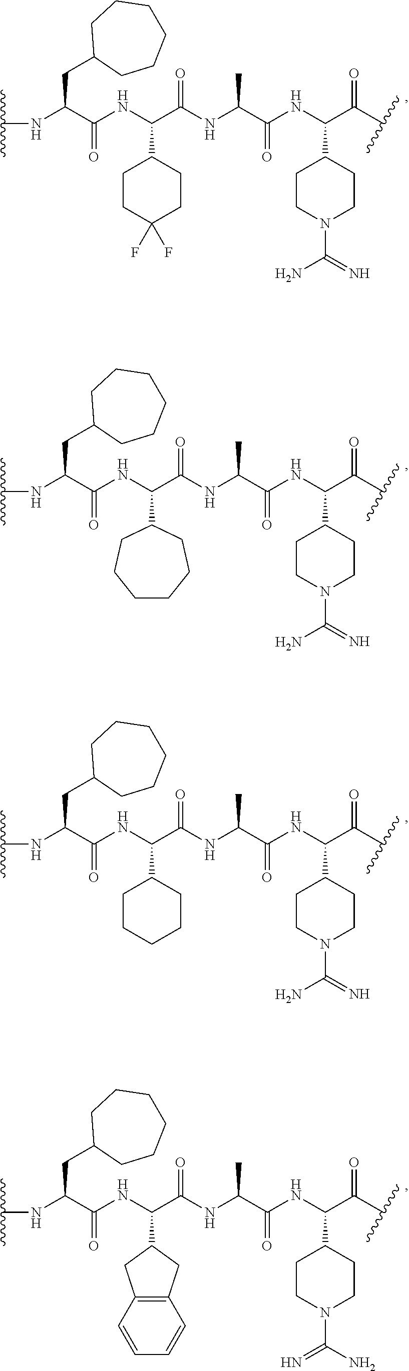 [SMILES: *N[C@@H](CC1CCCCCC1)C(=O)N[C@H](C(=O)N[C@@H](C)C(=O)N[C@H](C(*)=O)C1CCN(C(=N)N)CC1)C1CCC(F)(F)CC1 ; *N[C@@H](CC1CCCCCC1)C(=O)N[C@H](C(=O)N[C@@H](C)C(=O)N[C@H](C(*)=O)C1CCN(C(=N)N)CC1)C1CCCCCC1 ; *N[C@@H](CC1CCCCCC1)C(=O)N[C@H](C(=O)N[C@@H](C)C(=O)N[C@H](C(*)=O)C1CCN(C(=N)N)CC1)C1CCCCC1 ; *N[C@@H](CC1CCCCCC1)C(=O)N[C@H](C(=O)N[C@@H](C)C(=O)N[C@H](C(*)=O)C1CCN(C(=N)N)CC1)C1Cc2ccccc2C1]

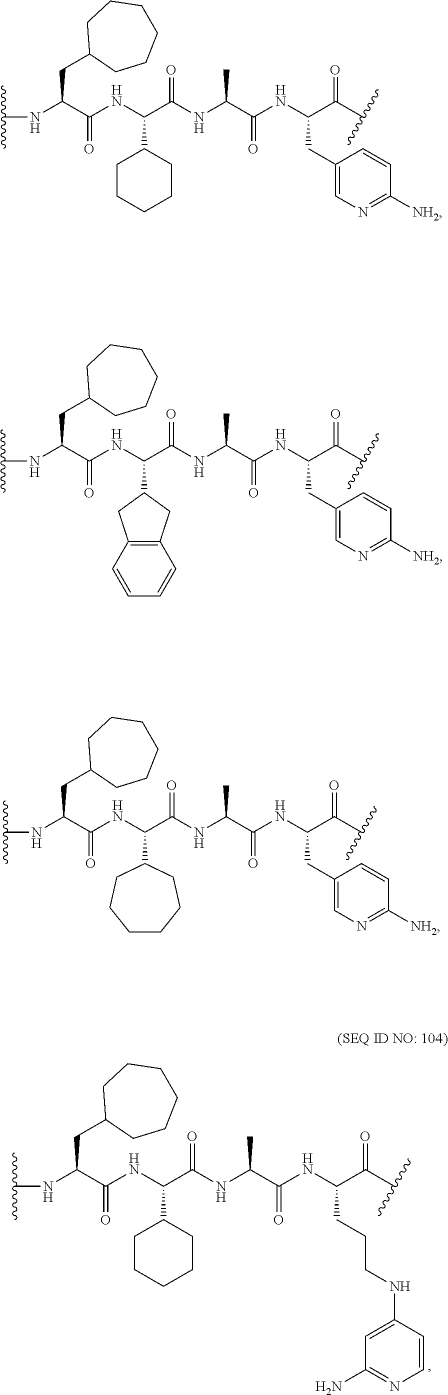 [SMILES: *N[C@@H](CC1CCCCCC1)C(=O)N[C@H](C(=O)N[C@@H](C)C(=O)N[C@@H](Cc1ccc(N)nc1)C(*)=O)C1CCCCC1 ; *N[C@@H](CC1CCCCCC1)C(=O)N[C@H](C(=O)N[C@@H](C)C(=O)N[C@@H](Cc1ccc(N)nc1)C(*)=O)C1Cc2ccccc2C1 ; *N[C@@H](CC1CCCCCC1)C(=O)N[C@H](C(=O)N[C@@H](C)C(=O)N[C@@H](Cc1ccc(N)nc1)C(*)=O)C1CCCCCC1 ; *N[C@@H](CC1CCCCCC1)C(=O)N[C@H](C(=O)N[C@@H](C)C(=O)N[C@@H](CCCNc1ccnc(N)c1)C(*)=O)C1CCCCC1]

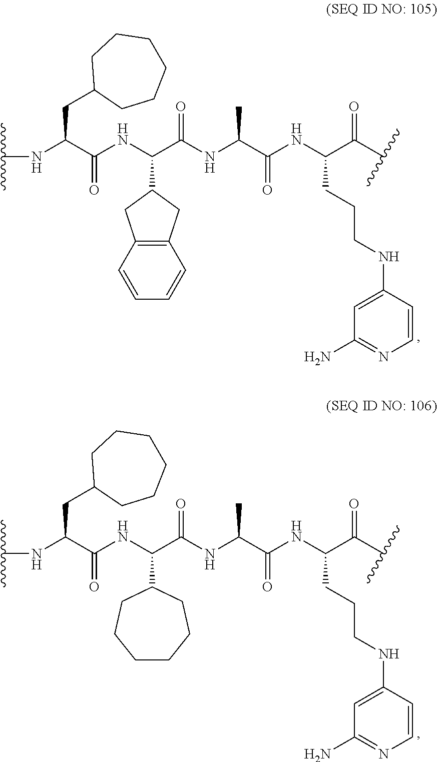 [SMILES: *N[C@@H](CC1CCCCCC1)C(=O)N[C@H](C(=O)N[C@@H](C)C(=O)N[C@@H](CCCNc1ccnc(N)c1)C(*)=O)C1Cc2ccccc2C1 ; *N[C@@H](CC1CCCCCC1)C(=O)N[C@H](C(=O)N[C@@H](C)C(=O)N[C@@H](CCCNc1ccnc(N)c1)C(*)=O)C1CCCCCC1]

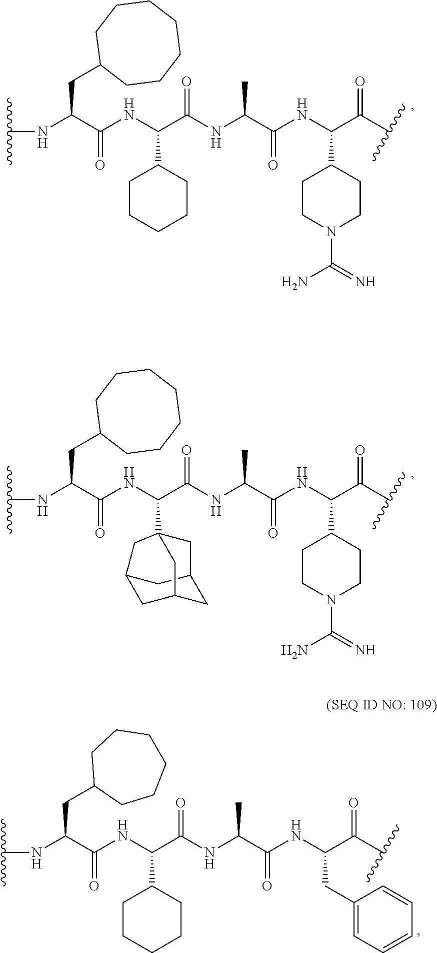 [SMILES: *N[C@@H](CC1CCCCCCC1)C(=O)N[C@H](C(=O)N[C@@H](C)C(=O)N[C@H](C(*)=O)C1CCN(C(=N)N)CC1)C1CCCCC1 ; *N[C@@H](CC1CCCCCCC1)C(=O)N[C@H](C(=O)N[C@@H](C)C(=O)N[C@H](C(*)=O)C1CCN(C(=N)N)CC1)C12CC3CC(CC(C3)C1)C2 ; *N[C@@H](CC1CCCCCC1)C(=O)N[C@H](C(=O)N[C@@H](C)C(=O)N[C@@H](Cc1ccccc1)C(*)=O)C1CCCCC1]

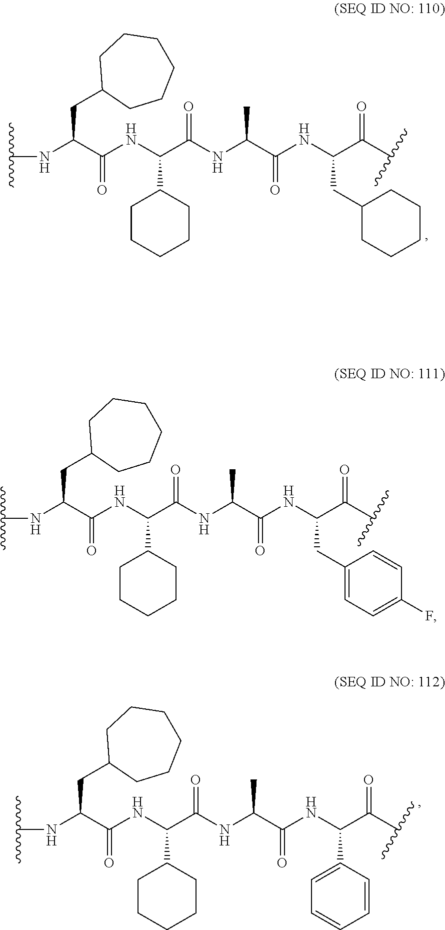 [SMILES: *N[C@@H](CC1CCCCCC1)C(=O)N[C@H](C(=O)N[C@@H](C)C(=O)N[C@@H](CC1CCCCC1)C(*)=O)C1CCCCC1 ; *N[C@@H](CC1CCCCCC1)C(=O)N[C@H](C(=O)N[C@@H](C)C(=O)N[C@@H](Cc1ccc(F)cc1)C(*)=O)C1CCCCC1 ; *N[C@@H](CC1CCCCCC1)C(=O)N[C@H](C(=O)N[C@@H](C)C(=O)N[C@H](C(*)=O)c1ccccc1)C1CCCCC1]

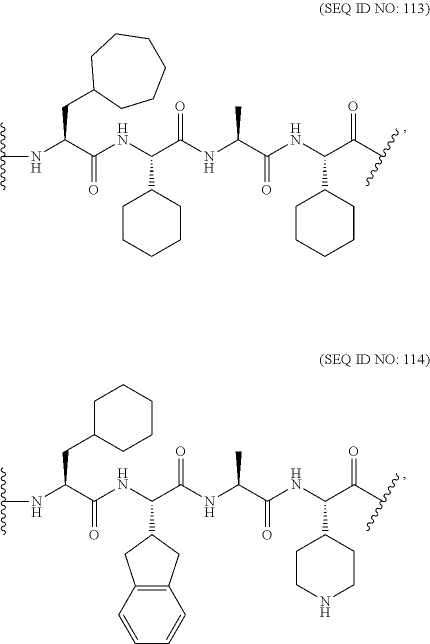 [SMILES: *N[C@@H](CC1CCCCCC1)C(=O)N[C@H](C(=O)N[C@@H](C)C(=O)N[C@H](C(*)=O)C1CCCCC1)C1CCCCC1 ; *N[C@@H](CC1CCCCC1)C(=O)N[C@H](C(=O)N[C@@H](C)C(=O)N[C@H](C(*)=O)C1CCNCC1)C1Cc2ccccc2C1]

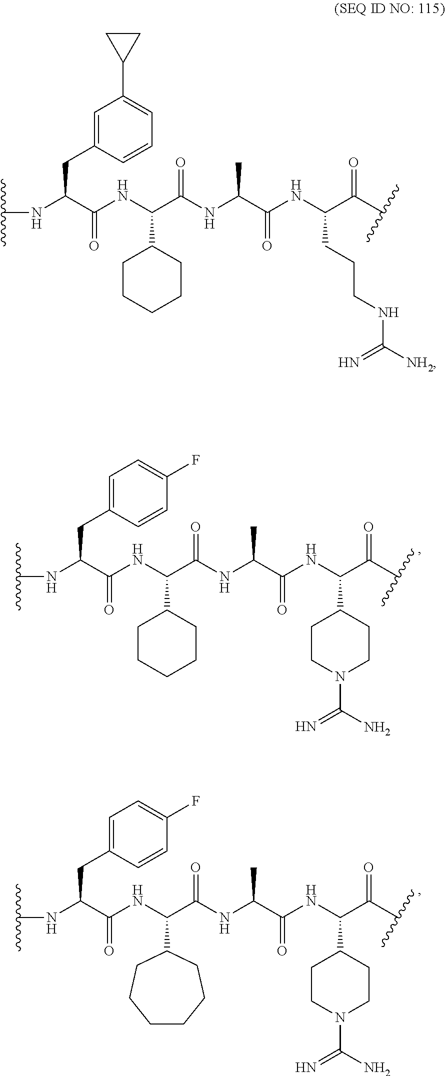 [SMILES: *N[C@@H](Cc1cccc(C2CC2)c1)C(=O)N[C@H](C(=O)N[C@@H](C)C(=O)N[C@@H](CCCNC(=N)N)C(*)=O)C1CCCCC1 ; *N[C@@H](Cc1ccc(F)cc1)C(=O)N[C@H](C(=O)N[C@@H](C)C(=O)N[C@H](C(*)=O)C1CCN(C(=N)N)CC1)C1CCCCC1 ; *N[C@@H](Cc1ccc(F)cc1)C(=O)N[C@H](C(=O)N[C@@H](C)C(=O)N[C@H](C(*)=O)C1CCN(C(=N)N)CC1)C1CCCCCC1]

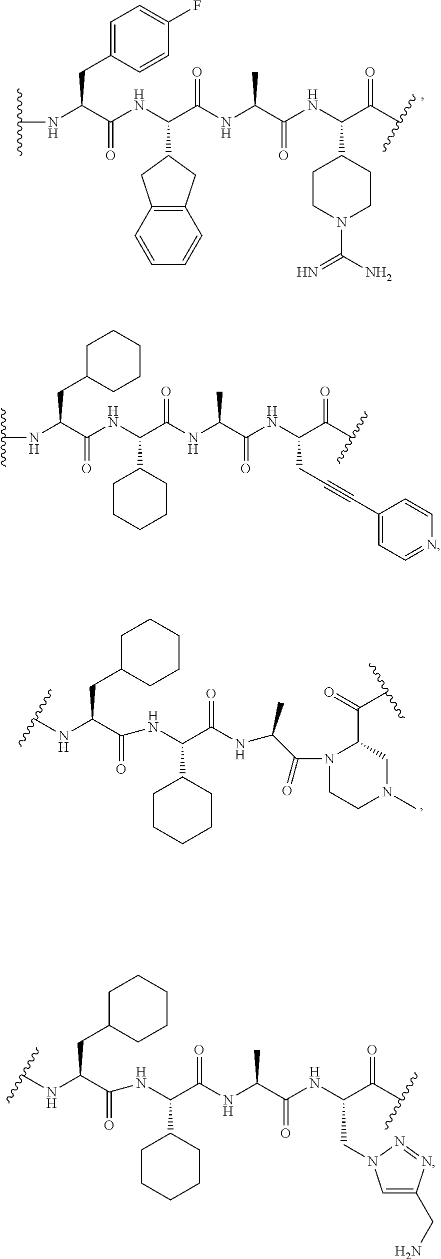 [SMILES: *N[C@@H](Cc1ccc(F)cc1)C(=O)N[C@H](C(=O)N[C@@H](C)C(=O)N[C@H](C(*)=O)C1CCN(C(=N)N)CC1)C1Cc2ccccc2C1 ; *N[C@@H](CC1CCCCC1)C(=O)N[C@H](C(=O)N[C@@H](C)C(=O)N[C@@H](CC#Cc1ccncc1)C(*)=O)C1CCCCC1 ; *N[C@@H](CC1CCCCC1)C(=O)N[C@H](C(=O)N[C@@H](C)C(=O)N1CCN(C)C[C@H]1C(*)=O)C1CCCCC1 ; *N[C@@H](CC1CCCCC1)C(=O)N[C@H](C(=O)N[C@@H](C)C(=O)N[C@@H](Cn1cc(CN)nn1)C(*)=O)C1CCCCC1]

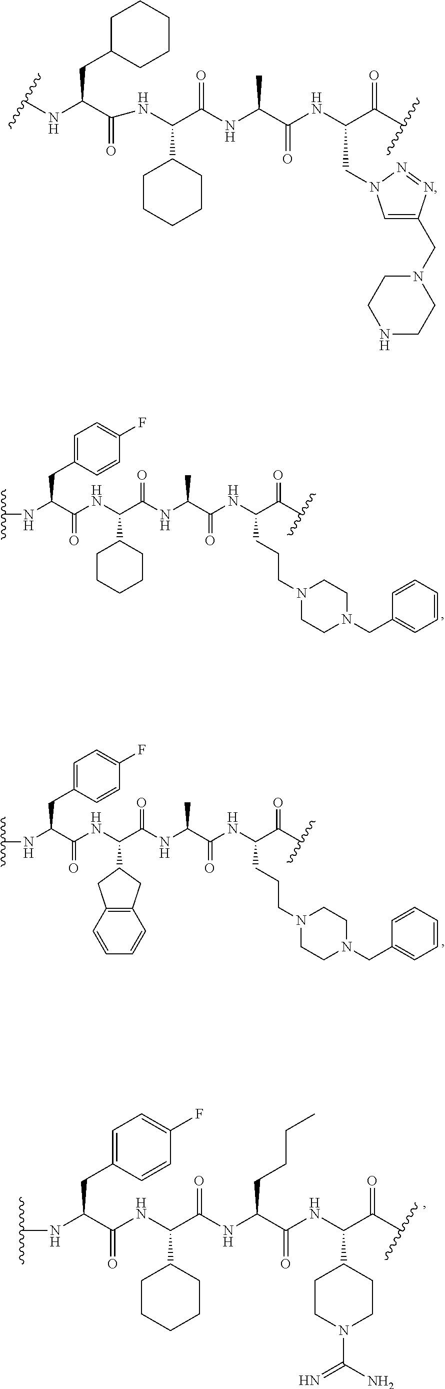 [SMILES: *N[C@@H](CC1CCCCC1)C(=O)N[C@H](C(=O)N[C@@H](C)C(=O)N[C@@H](Cn1cc(CN2CCNCC2)nn1)C(*)=O)C1CCCCC1 ; *N[C@@H](Cc1ccc(F)cc1)C(=O)N[C@H](C(=O)N[C@@H](C)C(=O)N[C@@H](CCCN1CCN(Cc2ccccc2)CC1)C(*)=O)C1CCCCC1 ; *N[C@@H](Cc1ccc(F)cc1)C(=O)N[C@H](C(=O)N[C@@H](C)C(=O)N[C@@H](CCCN1CCN(Cc2ccccc2)CC1)C(*)=O)C1Cc2ccccc2C1 ; *N[C@@H](Cc1ccc(F)cc1)C(=O)N[C@H](C(=O)N[C@@H](CCCC)C(=O)N[C@H](C(*)=O)C1CCN(C(=N)N)CC1)C1CCCCC1]

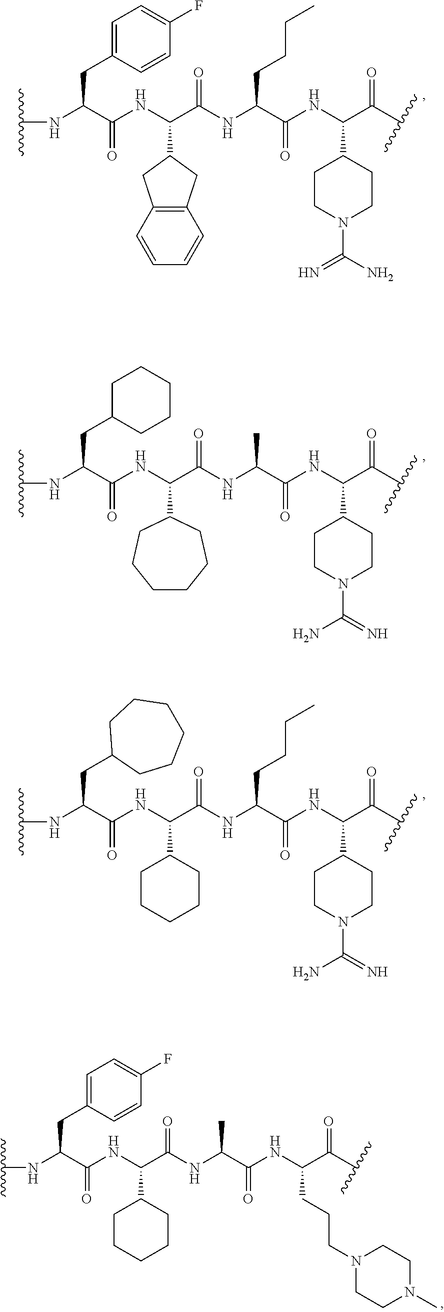 [SMILES: *N[C@@H](Cc1ccc(F)cc1)C(=O)N[C@H](C(=O)N[C@@H](CCCC)C(=O)N[C@H](C(*)=O)C1CCN(C(=N)N)CC1)C1Cc2ccccc2C1 ; *N[C@@H](CC1CCCCC1)C(=O)N[C@H](C(=O)N[C@@H](C)C(=O)N[C@H](C(*)=O)C1CCN(C(=N)N)CC1)C1CCCCCC1 ; *N[C@@H](CC1CCCCCC1)C(=O)N[C@H](C(=O)N[C@@H](CCCC)C(=O)N[C@H](C(*)=O)C1CCN(C(=N)N)CC1)C1CCCCC1 ; *N[C@@H](Cc1ccc(F)cc1)C(=O)N[C@H](C(=O)N[C@@H](C)C(=O)N[C@@H](CCCN1CCN(C)CC1)C(*)=O)C1CCCCC1]

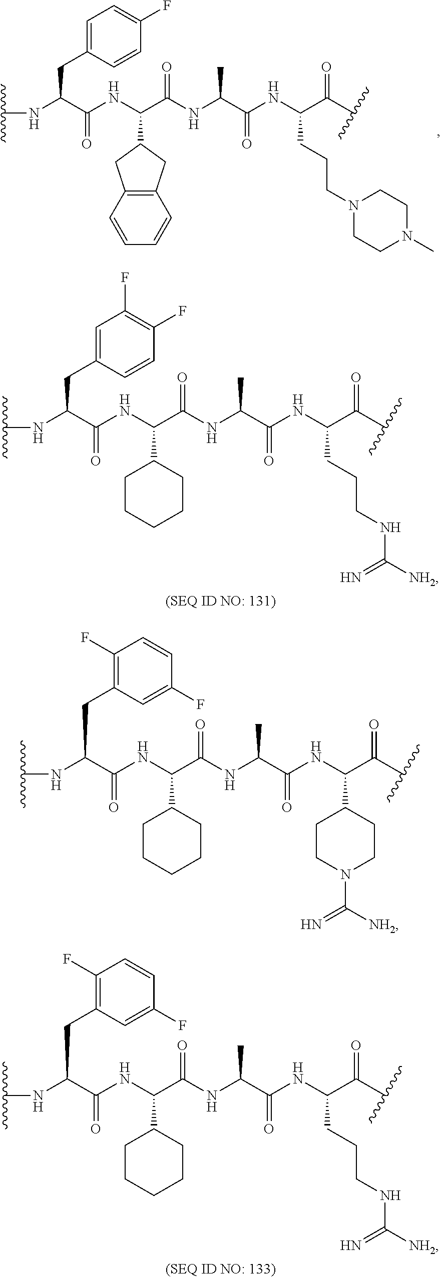

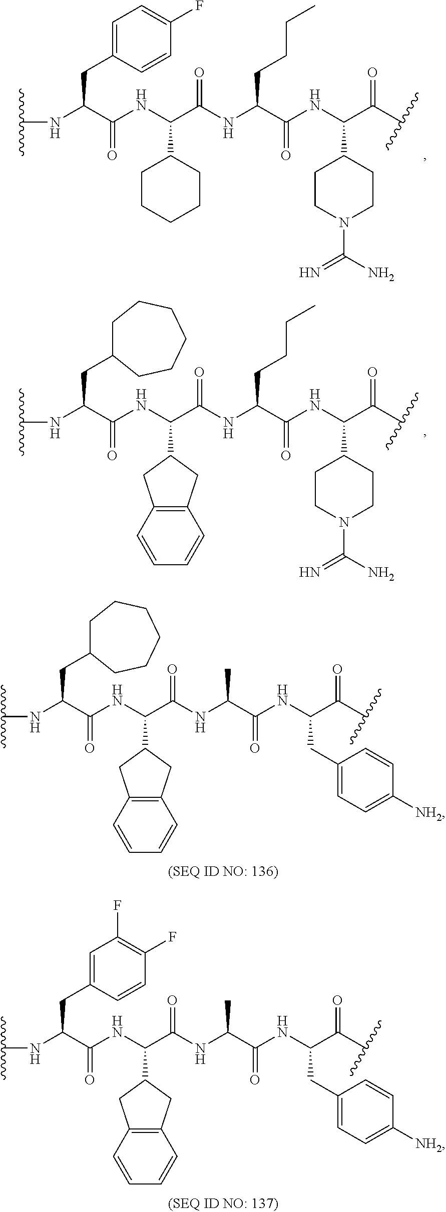

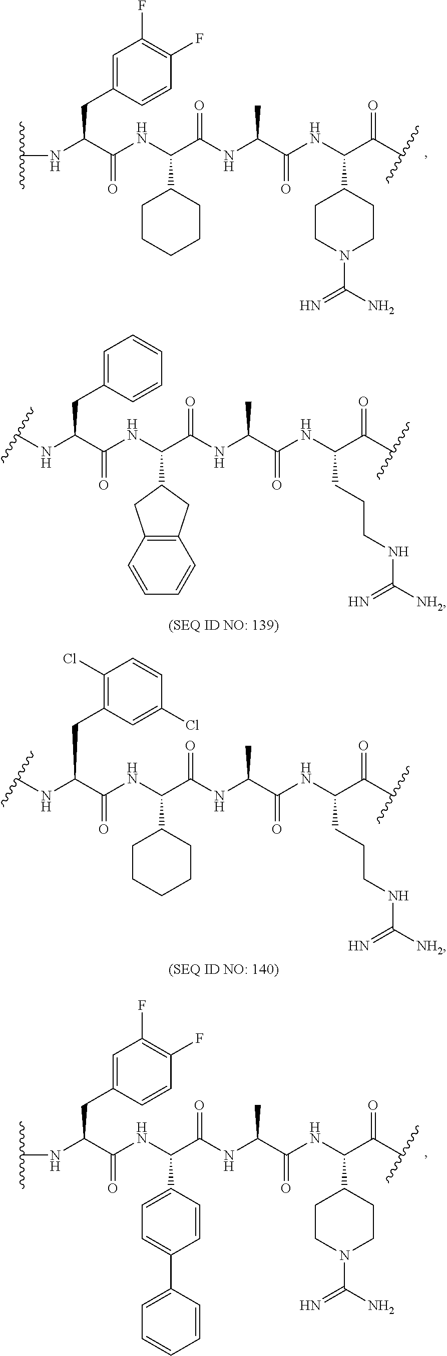 [SMILES: *N[C@@H](Cc1ccc(F)c(F)c1)C(=O)N[C@H](C(=O)N[C@@H](C)C(=O)N[C@H](C(*)=O)C1CCN(C(=N)N)CC1)C1CCCCC1 ; *N[C@@H](Cc1ccccc1)C(=O)N[C@H](C(=O)N[C@@H](C)C(=O)N[C@@H](CCCNC(=N)N)C(*)=O)C1Cc2ccccc2C1 ; *N[C@@H](Cc1cc(Cl)ccc1Cl)C(=O)N[C@H](C(=O)N[C@@H](C)C(=O)N[C@@H](CCCNC(=N)N)C(*)=O)C1CCCCC1 ; *N[C@@H](Cc1ccc(F)c(F)c1)C(=O)N[C@H](C(=O)N[C@@H](C)C(=O)N[C@H](C(*)=O)C1CCN(C(=N)N)CC1)c1ccc(-c2ccccc2)cc1]

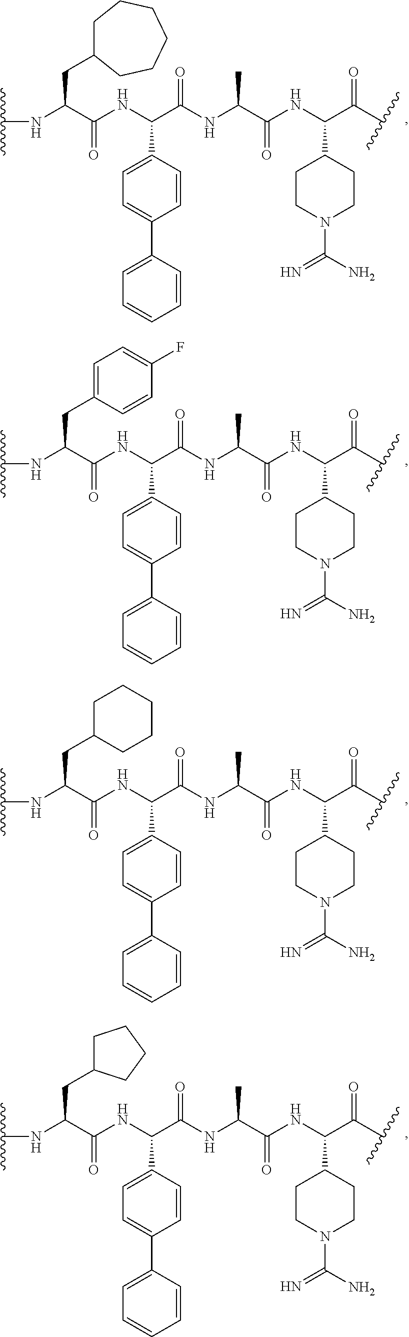

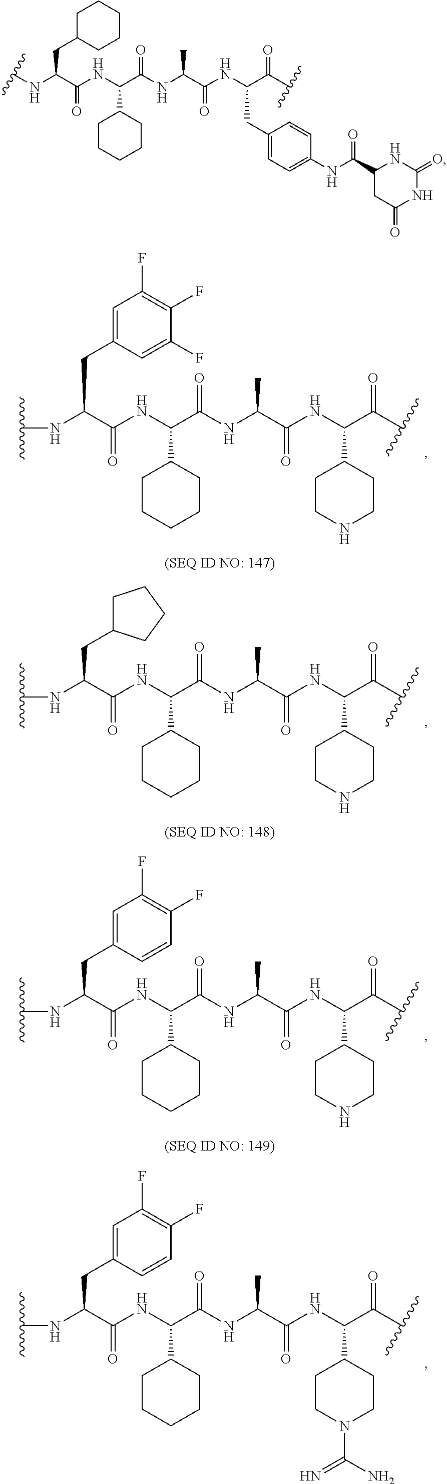

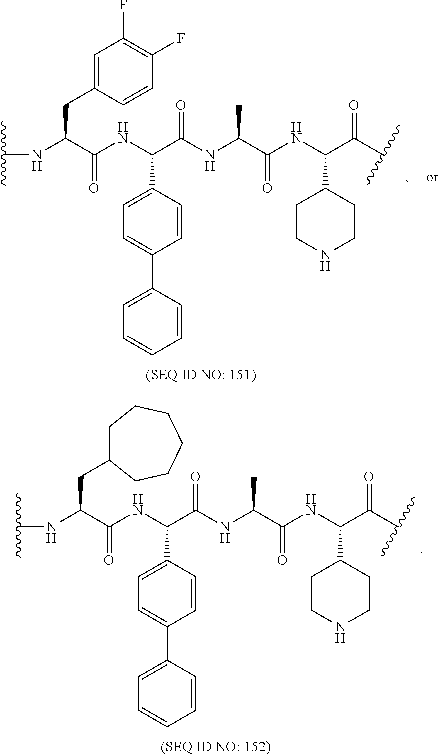 [SMILES: *N[C@@H](Cc1ccc(F)c(F)c1)C(=O)N[C@H](C(=O)N[C@@H](C)C(=O)N[C@H](C(*)=O)C1CCNCC1)c1ccc(-c2ccccc2)cc1 ; *N[C@@H](CC1CCCCCC1)C(=O)N[C@H](C(=O)N[C@@H](C)C(=O)N[C@H](C(*)=O)C1CCNCC1)c1ccc(-c2ccccc2)cc1]

Embodiment 36. The compound of any one of embodiments 1-35, or a pharmaceutically acceptable salt thereof, wherein: X⁵ is -L⁵-, (S)-alanine (Ala), (S)-lysine (Lys), (S)-ornithine (Orn), or (S)-(4-fluorobenzamido)methylglycine.

Embodiment 37. The compound of any one of embodiments 1-35, or a pharmaceutically acceptable salt thereof, wherein X⁵ is -L⁵-.

Embodiment 38. The compound of any one of embodiments 1-35, or a pharmaceutically acceptable salt thereof, wherein X⁵ is (S)-lysine (Lys) or (S)-ornithine (Orn).

Embodiment 39. The compound of any one of embodiments 1-35, or a pharmaceutically acceptable salt thereof, wherein X⁵ is (R)-lysine (Lys) or (R)-ornithine (Orn).

Embodiment 40. The compound of any one of embodiments 1-35, or a pharmaceutically acceptable salt thereof, wherein: X⁵ is (S)-(4-fluorobenzamido)methylglycine.

Embodiment 41. The compound of any one of embodiments 1-35, or a pharmaceutically acceptable salt thereof, wherein: X⁵ is N6-palmitoyl-(S)-lysine, N6-(15-carboxypentadecanoyl)-(S)-Lys, N6-(16-oxo-16-((2-(N—N—N-trimethyl-amino)ethyl)amino)hexadecanoyl)-(S)-Lys, N6-(4-fluorobenzoyl)-(S)-lysine, (S)-β³-homolysine, (S)-3-amino-alanine (H-Dap-OH), 4-fluoro-(S)-phenylalanine (Phe(4-F)), 4-iodo-(S)-phenylalanine (Phe(4-I)), 4-nitro-(S)-phenylalanine (Phe(4-NO₂)), (S)-(4-fluorobenzamido)methylglycine, 4-aminomethylphenyl-(S)-alanine (Phe(4-CH₂NH₂)), (S)-7-hydroxy-1,2,3,4-tetrahydroisoquinoline-3-carboxylic acid, (S)-proline, (S)-hydroxyproline (Hyp), (S)-piperidine-2-carboxylic acid, (S)-isoindoline-1-carboxylic acid, or (S)-(4-(aminomethyl)-1H-1,2λ²,3λ²-triazol-1-yl)methyl)-glycine.

Embodiment 42. The compound of any one of embodiments 1-21, or a pharmaceutically acceptable salt thereof, wherein:
X¹ is

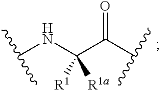

R¹ is -L¹-R^{1b};
   L¹ is —CH₂—;
   R^{1b} is cyclohexyl, cycloheptyl, or 4-fluorophenyl;
R^{1a} is hydrogen;
X² is

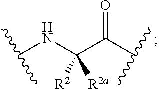

R² is -L²-R^{2b};
   L² is absent;
   R^{2b} is cyclohexyl, cycloheptyl, 2,3-dihydro-1H-inden-2-yl, or [1,1′-biphenyl]-4-yl;
R^{2a} is hydrogen;
X³ is (S)-alanine (Ala) or (S)-norleucine (Nle);
X⁴ is (S)-arginine (Arg), (S)-ornithine (Orn), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-3-(6-aminopyridin-3-yl)alanine ((3-Pal(6-NH₂)), or (S)-2-((2-aminopyridin-4-yl)-λ²-azaneyl)ethyl alanine; and X⁵ is -L⁵-, alanine (Ala), lysine (Lys), or ornithine (Orn).

Embodiment 43. The compound of any one of embodiments 1-42, or a pharmaceutically acceptable salt thereof, wherein X⁶ is absent.

Embodiment 44. The compound of any one of embodiments 1-42, or a pharmaceutically acceptable salt thereof, wherein X⁶ is -L⁶-.

Embodiment 45. The compound of any one of embodiments 1-42, or a pharmaceutically acceptable salt thereof, wherein X⁶ is (S)-lysine (Lys) or (S)-ornithine (Orn).

Embodiment 46. The compound of any one of embodiments 1-42, or a pharmaceutically acceptable salt thereof, wherein X⁶ is (R)-lysine (Lys) or (R)-ornithine (Orn).

Embodiment 47. The compound of any one of embodiments 1-42, or a pharmaceutically acceptable salt thereof, wherein X⁶ is

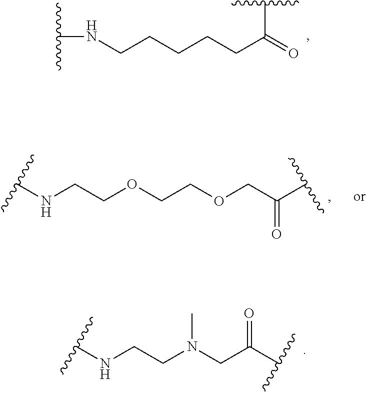

Embodiment 48. The compound of any one of embodiments 1-47, or a pharmaceutically acceptable salt thereof, wherein X⁷ is absent.

Embodiment 49. The compound of any one of embodiments 1-48, or a pharmaceutically acceptable salt thereof, wherein X⁷ is (S)-lysine (Lys) or (S)-ornithine (Orn).

Embodiment 50. The compound of any one of embodiments 1-49, wherein R^{a} is —C(═O)—R¹⁰, wherein R¹⁰ is a heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl, indolizinyl, indolyl, benzofuranyl, benzothiophenyl, indazolyl, benzimidazolyl, purinyl, quinolizinyl, quinolinyl, isoquinolinyl, cinnolinyl, phthalazinyl, quinazolinyl, quinoxalinyl, 1,8-naphthyridinyl, 1,7-naphthyridinyl, 1,6-naphthyridinyl, 1,5-naphthyridinyl, pteridinyl, and tetrazolo[1,5-a]pyridinyl; and wherein R¹⁰ is optionally substituted with 1 to 3 substituents that are each independently selected from the group consisting of halo, C₁-C₆ alkyl, alkoxy, —OH, —NH₂, —NH—C₁-C₆ alkylene-R¹¹, and —O—C₁-C₆ alkylene-R¹¹.

Embodiment 51. The compound of any one of embodiments 1-49, wherein R^{a} is —C(═O)—R¹⁰, wherein R¹⁰ is a monocyclic heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl; and wherein R¹⁰ is optionally substituted with 1 to 3 substituents that are each independently selected from the group consisting of halo, C₁-C₆ alkyl, alkoxy, —OH, —NH₂, —NH—C₁-C₆ alkylene-R¹¹, and —O—C₁-C₆ alkylene-R¹¹.

Embodiment 52. The compound of any one of embodiments 1-49, wherein R^{a} is —C(═O)—R¹⁰, wherein R¹⁰ is a monocyclic heteroaryl selected from the group consisting of pyridinyl, pyrimidinyl, pyrazinyl, and pyridazinyl; and wherein R¹⁰ is optionally substituted with —NH—C₁-C₆ alkylene-R¹¹, and —O—C₁-C₆ alkylene-R¹¹.

Embodiment 53. The compound of any one of embodiments 1-49, wherein R^{a} is —C(═O)—R¹¹, wherein R¹⁰ is a phenyl or naphthyl; and wherein R¹⁰ is optionally substituted with 1 to 3 substituents that are each independently selected from the group consisting of halo, C₁-C₆ alkyl, alkoxy, —OH, —NH₂, —NH—C₁-C₆ alkylene-R¹¹, and —O—C₁-C₆ alkylene-R¹¹.

Embodiment 54. The compound of any one of embodiments 1-49, wherein R^{a} is —C(═O)—R¹¹, wherein R¹⁰ is a partially unsaturated bicyclic heterocyclic ring; wherein any saturated carbon of R¹⁰ is optionally substituted with oxo (═O); and wherein R¹⁰ is optionally substituted with 1 to 3 substituents that are each independently selected from the group consisting of halo, C₁-C₆ alkyl, alkoxy, —OH, and —NH₂.

Embodiment 55. The compound of any one of embodiments 1-49, wherein R^{a} is

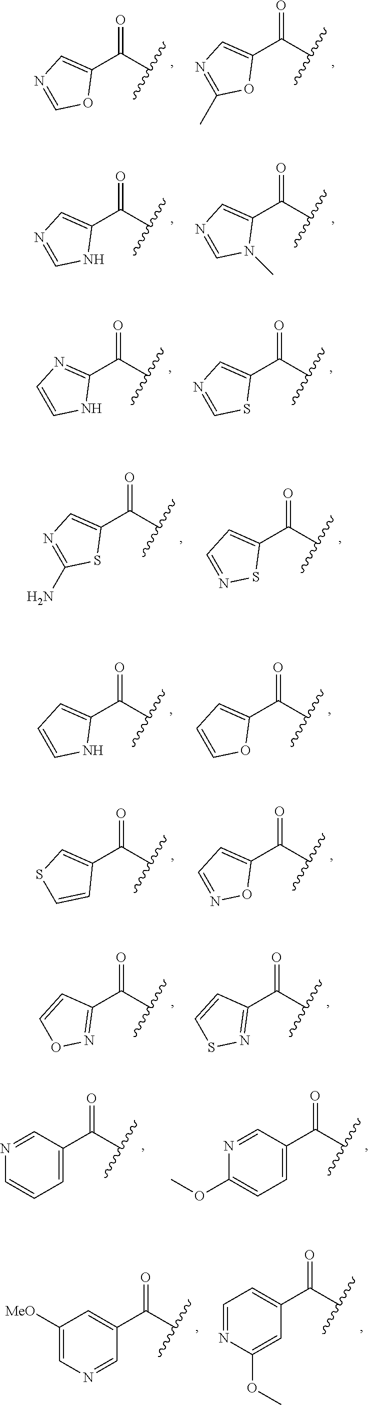

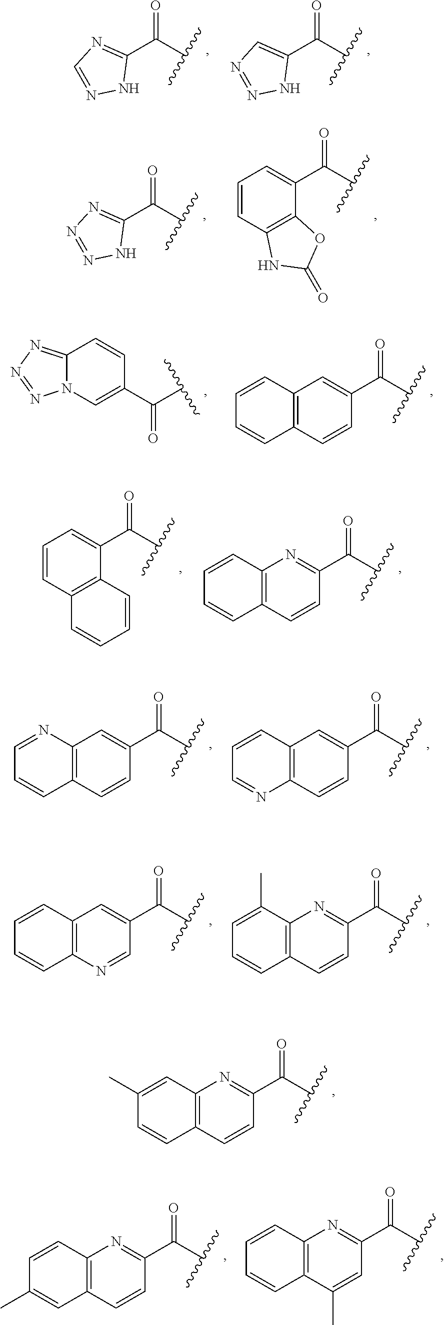

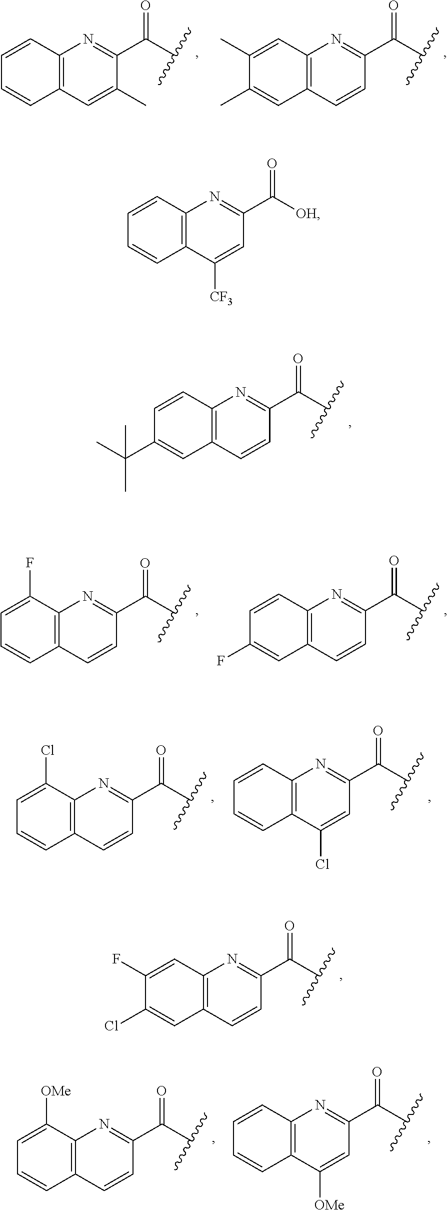

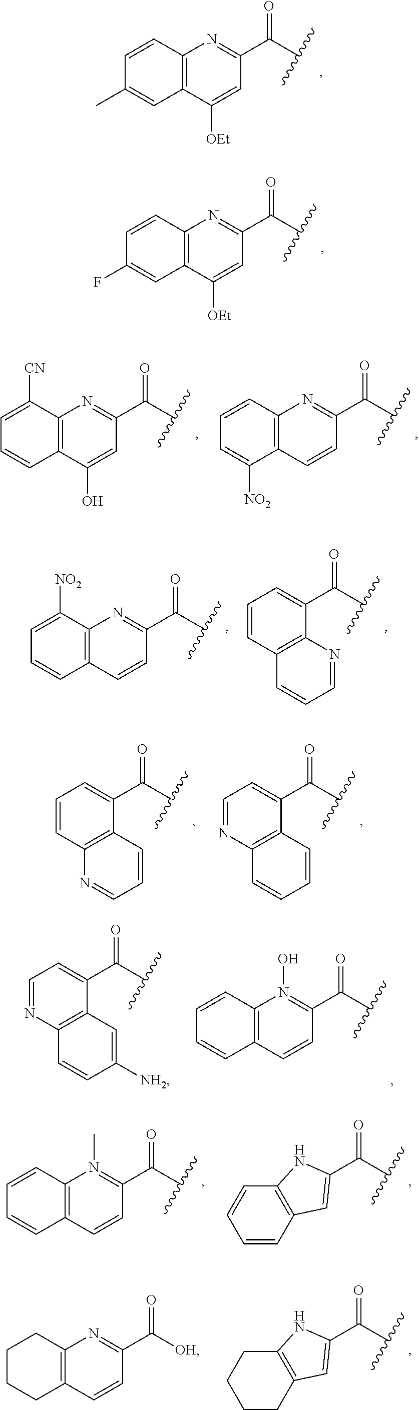

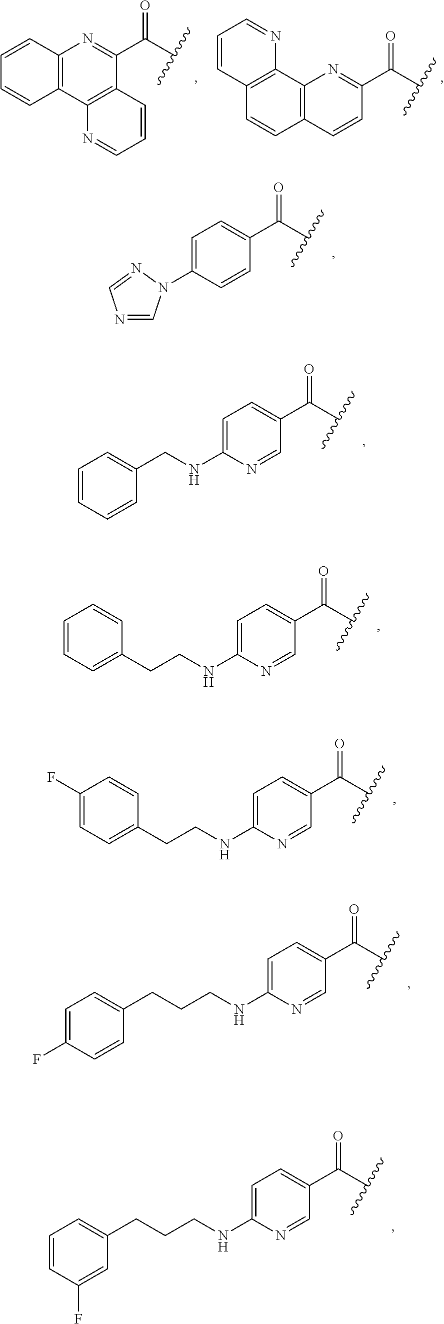

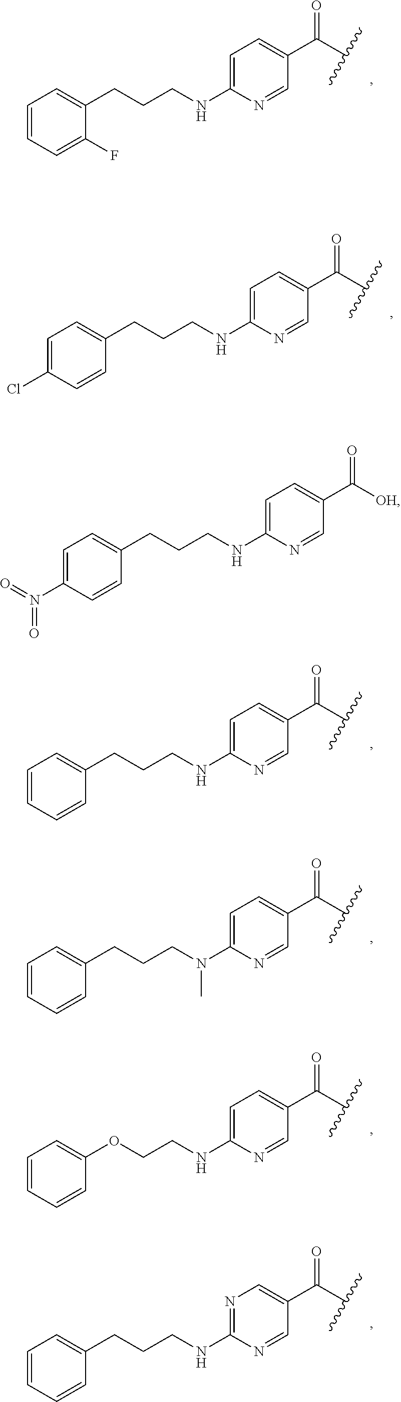

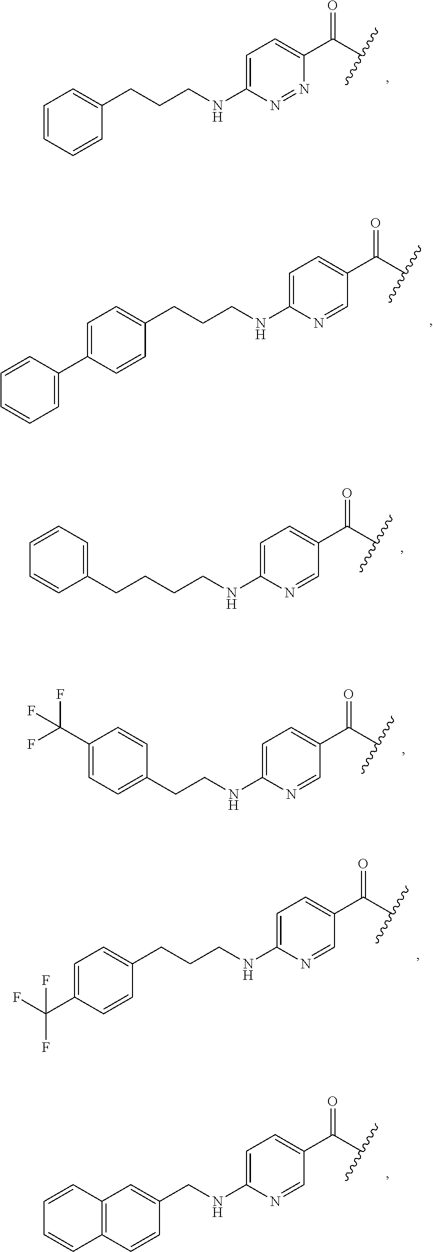 [SMILES: *C(=O)c1ccc(NCCCc2ccccc2)nn1 ; *C(=O)c1ccc(NCCCc2ccc(-c3ccccc3)cc2)nc1 ; *C(=O)c1ccc(NCCCCc2ccccc2)nc1 ; *C(=O)c1ccc(NCCc2ccc(C(F)(F)F)cc2)nc1 ; *C(=O)c1ccc(NCCCc2ccc(C(F)(F)F)cc2)nc1 ; *C(=O)c1ccc(NCc2ccc3ccccc3c2)nc1]

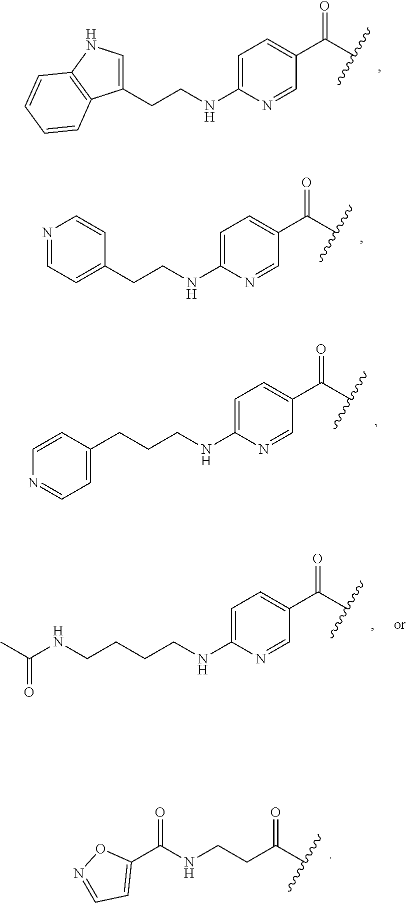 [SMILES: *C(=O)c1ccc(NCCc2c[nH]c3ccccc23)nc1 ; *C(=O)c1ccc(NCCc2ccncc2)nc1 ; *C(=O)c1ccc(NCCCc2ccncc2)nc1 ; *C(=O)c1ccc(NCCCCNC(C)=O)nc1 ; *C(=O)CCNC(=O)c1ccno1]

Embodiment 56 The compound of any one of embodiments 1-49, wherein R^{a} is

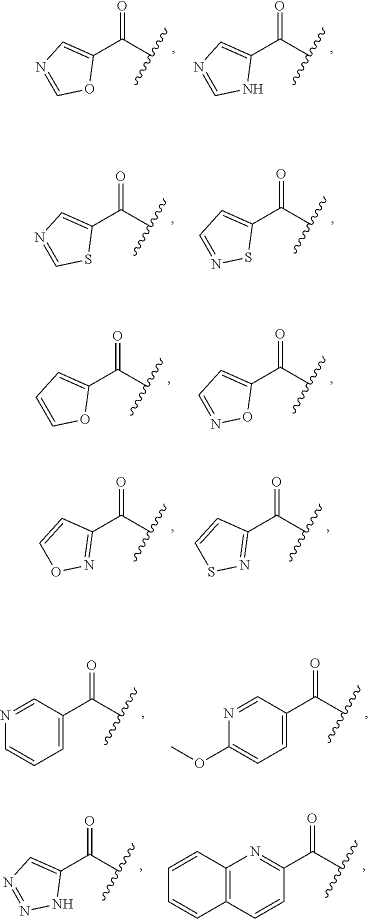

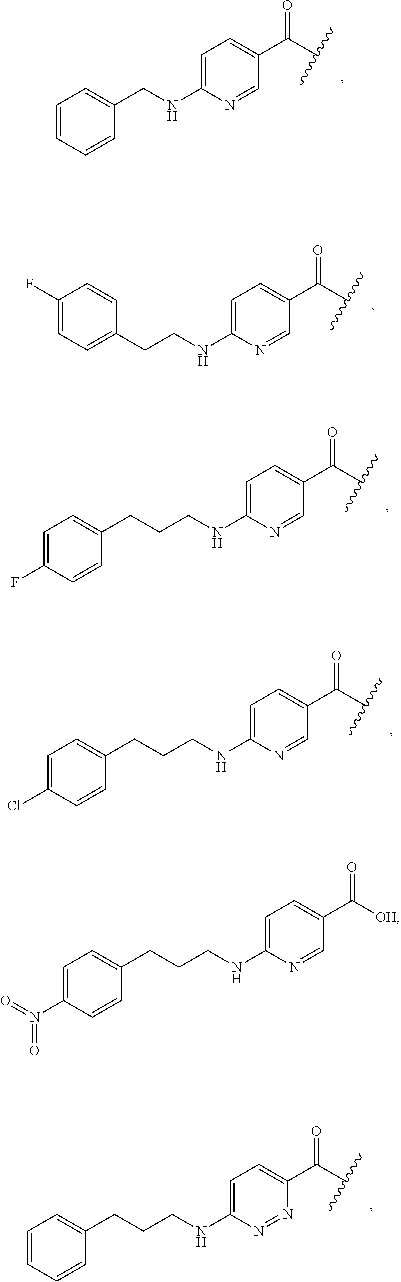 [SMILES: *C(=O)c1ccc(NCc2ccccc2)nc1 ; *C(=O)c1ccc(NCCc2ccc(F)cc2)nc1 ; *C(=O)c1ccc(NCCCc2ccc(F)cc2)nc1 ; *C(=O)c1ccc(NCCCc2ccc(Cl)cc2)nc1 ; O=C(O)c1ccc(NCCCc2ccc([N+](=O)[O-])cc2)nc1 ; *C(=O)c1ccc(NCCCc2ccccc2)nn1]

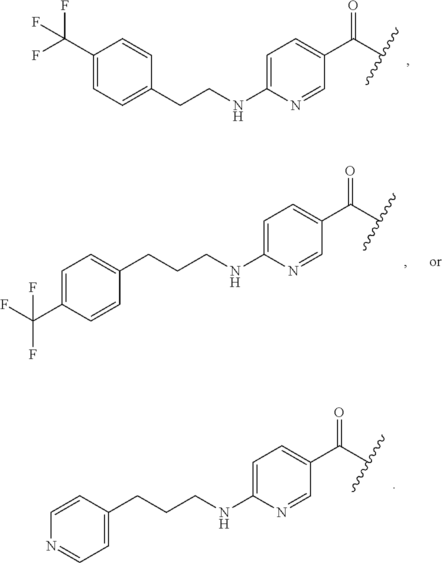 [SMILES: *C(=O)c1ccc(NCCc2ccc(C(F)(F)F)cc2)nc1 ; *C(=O)c1ccc(NCCCc2ccc(C(F)(F)F)cc2)nc1 ; *C(=O)c1ccc(NCCCc2ccncc2)nc1]

Embodiment 57. The compound of any one of embodiments 1-49, wherein R^{a} is

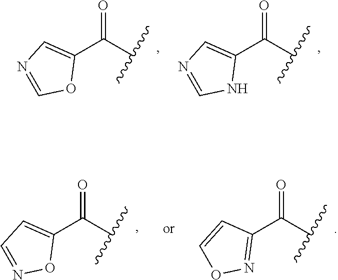 [SMILES: *C(=O)c1cnco1 ; *C(=O)c1cnc[nH]1 ; *C(=O)c1ccno1 ; *C(=O)c1ccon1]

Embodiment 58. The compound of embodiment 1, wherein

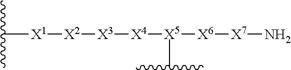

is

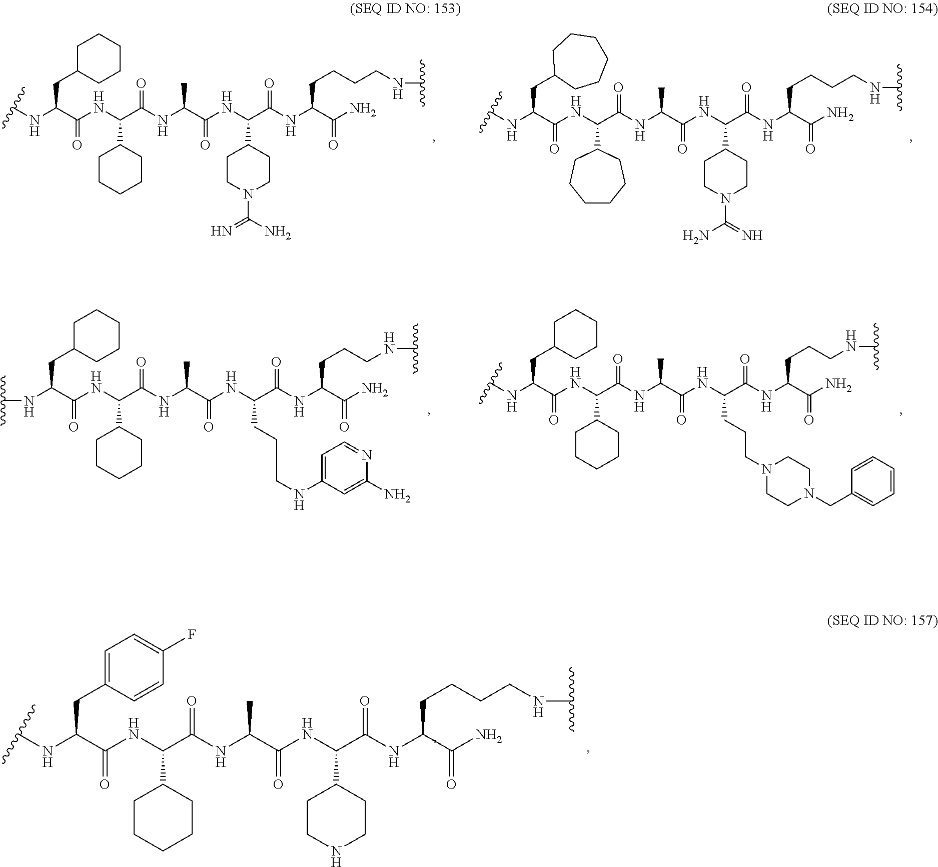

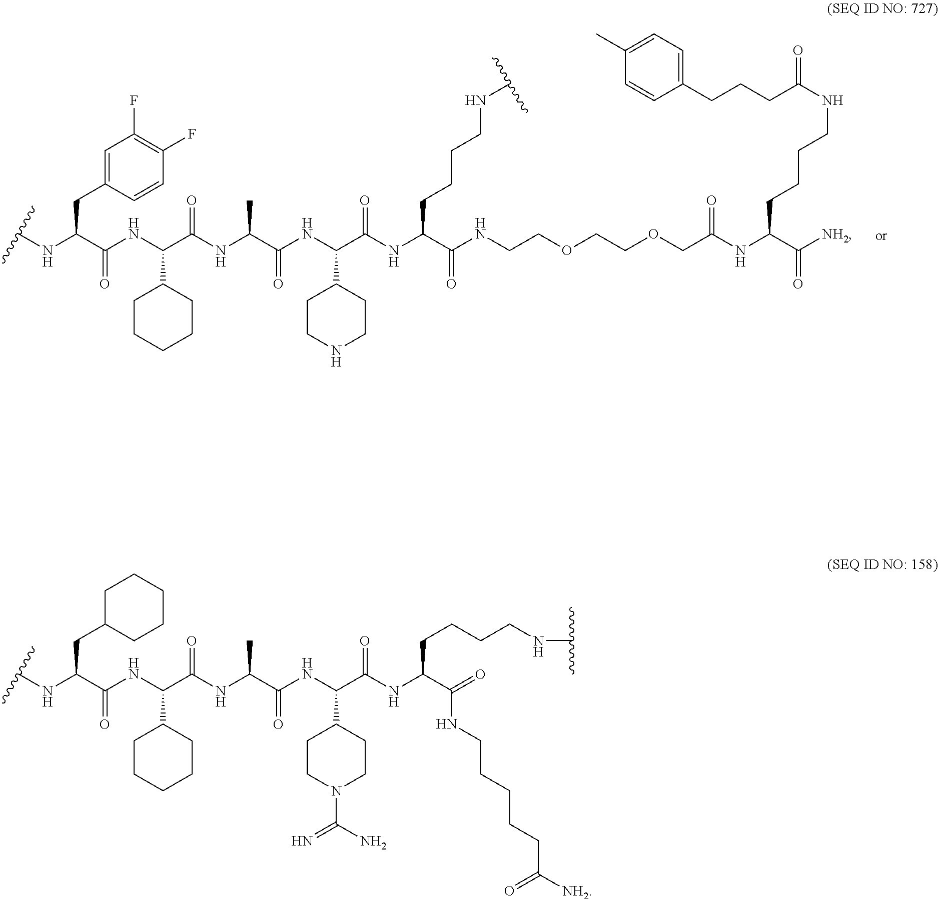 [SMILES: *NCCCC[C@H](NC(=O)[C@@H](NC(=O)[C@H](C)NC(=O)[C@@H](NC(=O)[C@H](Cc1ccc(F)c(F)c1)N*)C1CCCCC1)C1CCNCC1)C(=O)NCCOCCOCC(=O)N[C@@H](CCCCNC(=O)CCCc1ccc(C)cc1)C(N)=O ; *NCCCC[C@H](NC(=O)[C@@H](NC(=O)[C@H](C)NC(=O)[C@@H](NC(=O)[C@H](CC1CCCCC1)N*)C1CCCCC1)C1CCN(C(=N)N)CC1)C(=O)NCCCCCC(N)=O]

Embodiment 59. The compound of embodiment 1, wherein

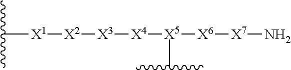

is:

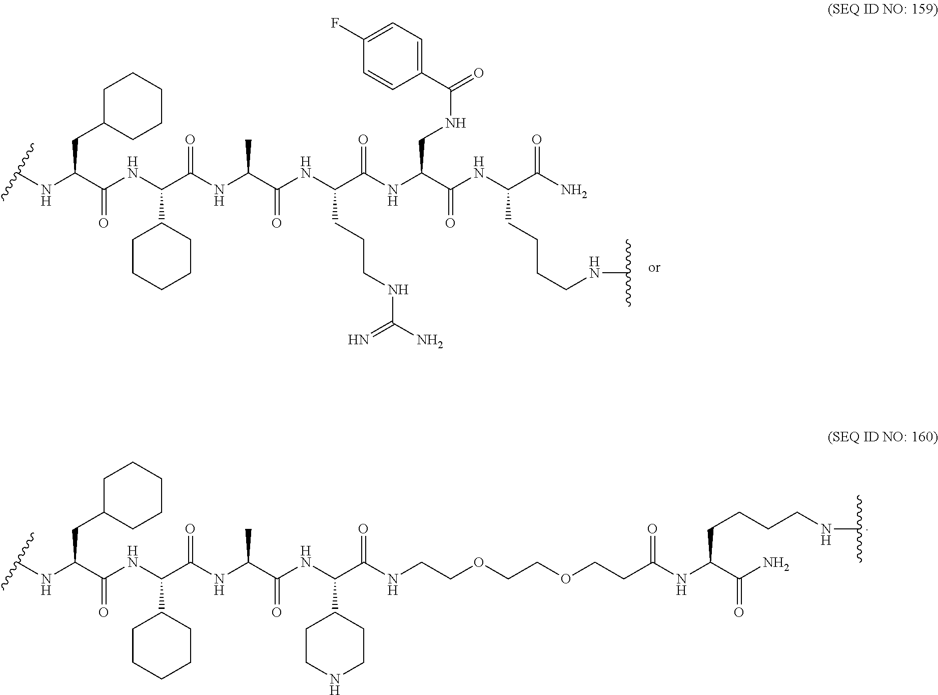 [SMILES: *NCCCC[C@H](NC(=O)[C@H](CNC(=O)c1ccc(F)cc1)NC(=O)[C@H](CCCNC(=N)N)NC(=O)[C@H](C)NC(=O)[C@@H](NC(=O)[C@H](CC1CCCCC1)N*)C1CCCCC1)C(N)=O ; *NCCCC[C@H](NC(=O)CCOCCOCCNC(=O)[C@@H](NC(=O)[C@H](C)NC(=O)[C@@H](NC(=O)[C@H](CC1CCCCC1)N*)C1CCCCC1)C1CCNCC1)C(N)=O]

Embodiment 60. The compound of any one of embodiments 1-59, or a pharmaceutically acceptable salt thereof, wherein R^{b} is a chelating moiety independently selected from the group consisting of:
1,4,7,10-tetraazacyclododecane-1,4,7,10-tetraacetic acid (DOTA);
2,2′,2″-(10-(2-amino-2-oxoethyl)-1,4,7,10-tetraazacyclododecane-1,4,7-triyl)triacetic acid (PSC);
1,4,7,10-tetraazacyclododecane-1,4,7-triacetic acid (DO3A);
1,4,7,10-tetraazacyclododecane-1,7-diacetic acid (DO2A);
α,α′,α″,α′″-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid;
benzyl-1,4,7,10-tetraazacyclododecane-1,4,7,10-tetraacetic acid (Bn-DOTA);
p-hydroxy-benzyl-1,4,7,10-tetraazacyclododecane-1,4,7,10-tetraacetic acid (p-OH-Bn-DOTA);
6,6′-(((pyridine-2,6-diylbis(methylene))bis((carboxymethyl)azanediyl))bis(methylene))dipicolinic acid (H₄pypa);
H1pypa-benzyl;
6,6′,6″,6″′-(((pyridine-2,6-diylbis(methylene))bis(azanetriyl))tetrakis(methylene))-tetrapicolinic acid (H₄py4pa);
H₄py4pa-benzyl;
2,2′,2″-(1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2,2′,2″,2′″-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H4octapa-benzyl; and
3,6,9,12-tetrakis(carboxymethyl)-3,6,9,12-tetraazatetradecanedioic acid (TTHA);
or a radionuclide complex thereof.

Embodiment 61. The compound of any one of embodiments 1-59, or a pharmaceutically acceptable salt thereof, wherein R^{b} is a chelating moiety selected from the group consisting of:
1,4,7,10-tetraazacyclododecane-1,4,7,10-tetraacetic acid (DOTA) and
1,4,7,10-tetraazacyclododecane-1,4,7-triacetic acid (DO3A); or a radionuclide complex thereof.

Embodiment 62. The compound of any one of embodiments 1-59, or a pharmaceutically acceptable salt thereof, wherein Rbis a chelating moiety independently selected from the group consisting of:

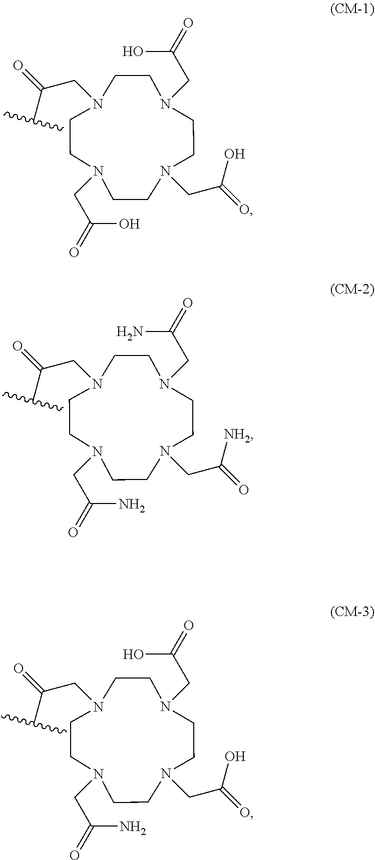 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1]

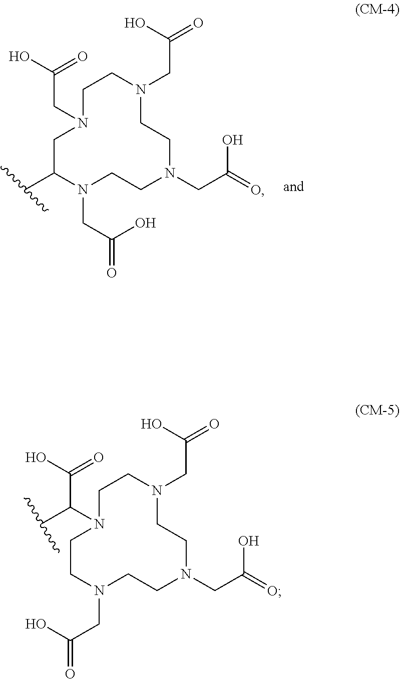 [SMILES: *C1CN(CC(=O)O)CCN(CC(=O)O)CCN(CC(=O)O)CCN1CC(=O)O ; "and" ; *C(C(=O)O)N1CCN(CC(=O)O)CCN(CC(=O)O)CCN(CC(=O)O)CC1]

or a radionuclide complex thereof.

Embodiment 63. The compound of any one of embodiments 1-59, or a pharmaceutically acceptable salt thereof, wherein R^{b} is

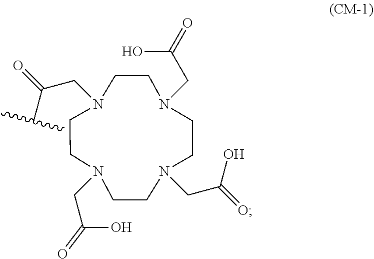 [SMILES: *C(=O)CN1CCN(CC(=O)O)CCN(CC(=O)O)CCN(CC(=O)O)CC1]

or a radionuclide complex thereof.

Embodiment 64. The compound of any one of embodiments 1-59, or a pharmaceutically acceptable salt thereof, wherein Rbis independently selected from:

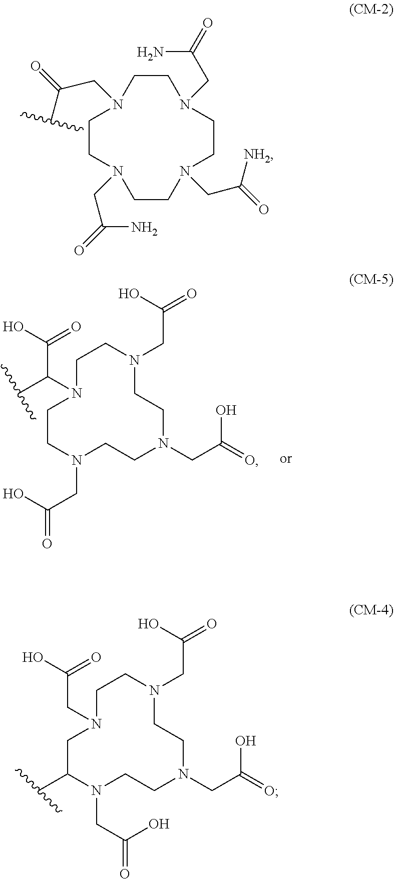 [SMILES: *C(=O)CN1CCN(CC(N)=O)CCN(CC(N)=O)CCN(CC(N)=O)CC1 ; *C(C(=O)O)N1CCN(CC(=O)O)CCN(CC(=O)O)CCN(CC(=O)O)CC1 ; "or" ; *C1CN(CC(=O)O)CCN(CC(=O)O)CCN(CC(=O)O)CCN1CC(=O)O]

or a radionuclide complex thereof.

Embodiment 65. The compound of any one of embodiments 1-64, or a pharmaceutically acceptable salt thereof, wherein L⁹ is: —C(═O)-L¹⁰-L¹¹-L¹² or —CH₂—C(═O)-L¹⁰-L¹-L¹²-;
L¹⁰ is absent, —C₁-C₆ alkylene, —NR¹³—, substituted or unsubstituted heterocycloalkylene, or substituted or unsubstituted cyclohexylene;
L¹¹ is absent, —C₁-C₆ alkylene, substituted or unsubstituted heterocycloalkylene, substituted or unsubstituted cyclohexylene, or —(OCH₂CH₂)ₓ—;
L¹² is —NR¹⁴—;
R¹³ and R¹⁴ are each independently selected from H or —C₁-C₄ alkyl; and
x is 1, 2, 3, 4, 5, or 6.

Embodiment 66. The compound of any one of embodiments 1-64, or a pharmaceutically acceptable salt thereof, wherein L⁹ is: —C(═O)—C₁-C₆ alkylene-NR¹⁴—*, —C(═O)—NR¹—C₁-C₆ alkylene-NR¹⁴—*, —C(═O)—C₁-C₆ alkylene-(substituted or unsubstituted heterocycloalkylene)-NR¹⁴—*, —C(═O)-(substituted or unsubstituted heterocycloalkylene)-C₁-C₆ alkylene-NR¹⁴—*, —C(═O)—C₁-C₆ alkylene-(substituted or unsubstituted cyclohexylene)-NR¹⁴—*, —C(═O)-(substituted or unsubstituted cyclohexylene)-C₁-C₆ alkylene-NR¹⁴—*, —C(═O)—C₁-C₆ alkylene-(OCH₂CH₂)ₓ—NR¹⁴—*; —CH₂—C(═O)—C₁-C₆ alkylene-NR¹⁴—*, —CH₂—C(═O)—NR¹³—C₁-C₆ alkylene-NR¹⁴—*, —CH₂—C(═O)—C₁-C₆ alkylene-(substituted or unsubstituted heterocycloalkylene)-NR¹⁴—*, —CH₂—C(═O)-(substituted or unsubstituted heterocycloalkylene)-C₁-C₆ alkylene-NR¹⁴—*, —CH₂—C(═O)—C₁-C₆ alkylene-(substituted or unsubstituted cyclohexylene)-NR¹⁴—*, —CH₂—C(═O)-(substituted or unsubstituted cyclohexylene)-C₁-C₆ alkylene-NR¹⁴—*, or —CH₂—C(═O)—C₁-C₆ alkylene-(OCH₂CH₂)ₓ—NR¹⁴—*;
R¹³ and R¹⁴ are each independently selected from H or —C₁-C₄ alkyl; and
x is 1, 2, 3, 4, 5, or 6;
wherein * denotes the attachment point to R^{b}.

Embodiment 67. The compound of any one of embodiments 1-64, or a pharmaceutically acceptable salt thereof, wherein L⁹ is absent.

Embodiment 68. The compound of any one of embodiments 1-64, or a pharmaceutically acceptable salt thereof, wherein L⁹ is:

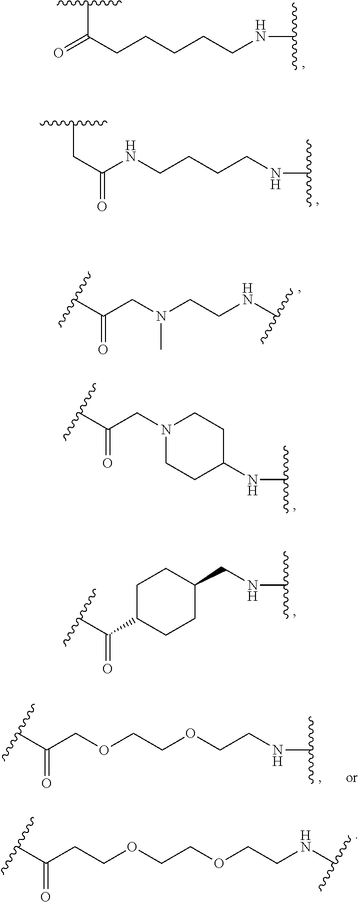

Embodiment 69. The compound of any one of embodiments 1-68, or a pharmaceutically acceptable salt thereof, wherein -L⁵-, -L⁶-, or -L⁷- are each independently selected from the group consisting of: —NR¹⁵—C₁-C₆ alkyl-C(═O)—, —NR¹⁵—(CH₂CH₂O)—C₁-C₆ alkyl-C(═O)—, —NR¹⁵(CH₂CH₂NR¹⁶)_{y}—C₁-C₆ alkyl-C(═O)—, —NR¹⁵—C₁-C₆ alkyl-(substituted or unsubstituted heterocycloalkyl)-C(═O)—, —NR¹⁵-(substituted or unsubstituted heterocycloalkyl)-C₁-C₆ alkyl-C(═O)—, —NR¹⁵—C₁-C₆ alkyl-(substituted or unsubstituted cyclohexyl)-C(═O)—, and —NR¹⁵-(substituted or unsubstituted cyclohexyl)-C₁-C₆ alkyl-C(═O)—;
R¹⁵ and R¹⁶ are each independently selected from H or —C₁-C₄ alkyl; and
y is 1, 2, 3, 4, 5, or 6.

Embodiment 70. The compound of any one of embodiments 1-68, or a pharmaceutically acceptable salt thereof, wherein the -L⁵-, -L⁶-, or -L⁷- are each independently selected from the group consisting of: —NH—C₁-C₆ alkylene-C(═O)—, —NH—(C₂CH₂O)_{y}—C₁-C₆ alkylene-C(═O)—, —NH(CH₂CH₂NR¹⁶)_{y}—C₁-C₆alkylene-C(═O)—, —NH—C₁-C₆alkylene-(unsubstituted piperidinylene)-C(═O)—, —NH-(unsubstituted piperidinylene)-C₁-C₆ alkylene-C(═O)—, —NH—C₁-C₆ alkylene-(unsubstituted cyclohexylene)-C(═O)—, and —NH-(unsubstituted cyclohexylene)-C₁-C₆ alkylene-C(═O)—.

Embodiment 71. The compound of any one of embodiments 1-68, or a pharmaceutically acceptable salt thereof, wherein -L⁵-, -L⁶-, or -L⁷- are each independently selected from the group consisting of:

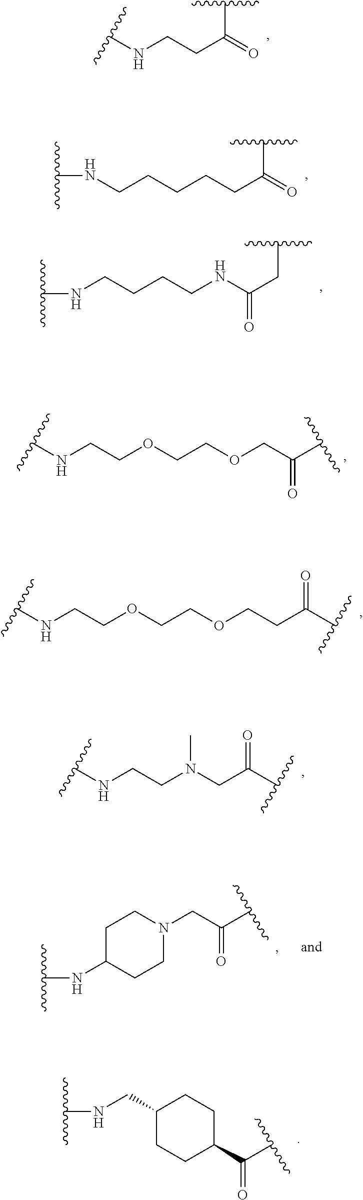

Embodiment 72. The compound of embodiment 1, or a pharmaceutically acceptable salt thereof, wherein the compound of Formula (I) has one of the following chemical structures, or a pharmaceutically acceptable salt thereof:

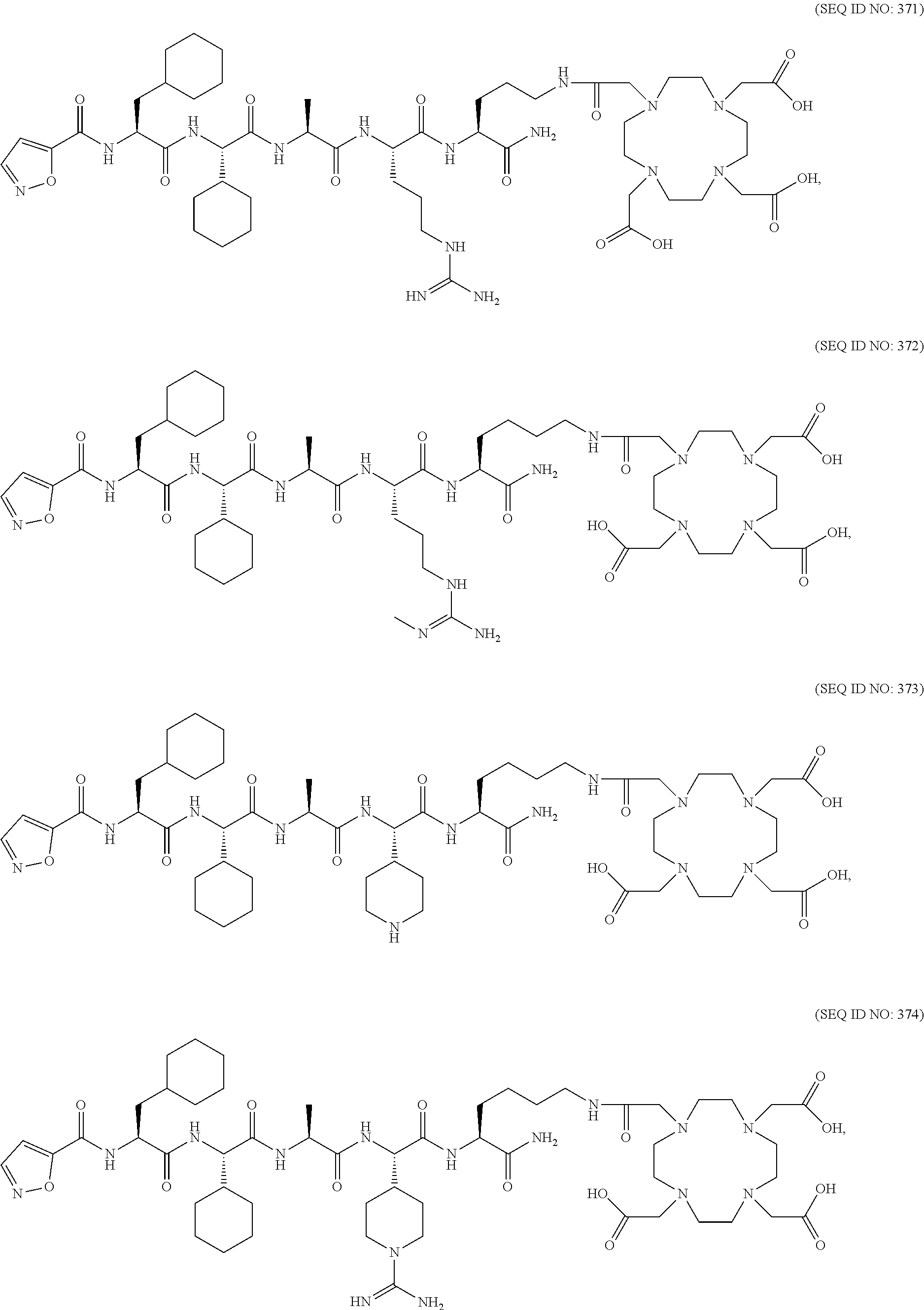

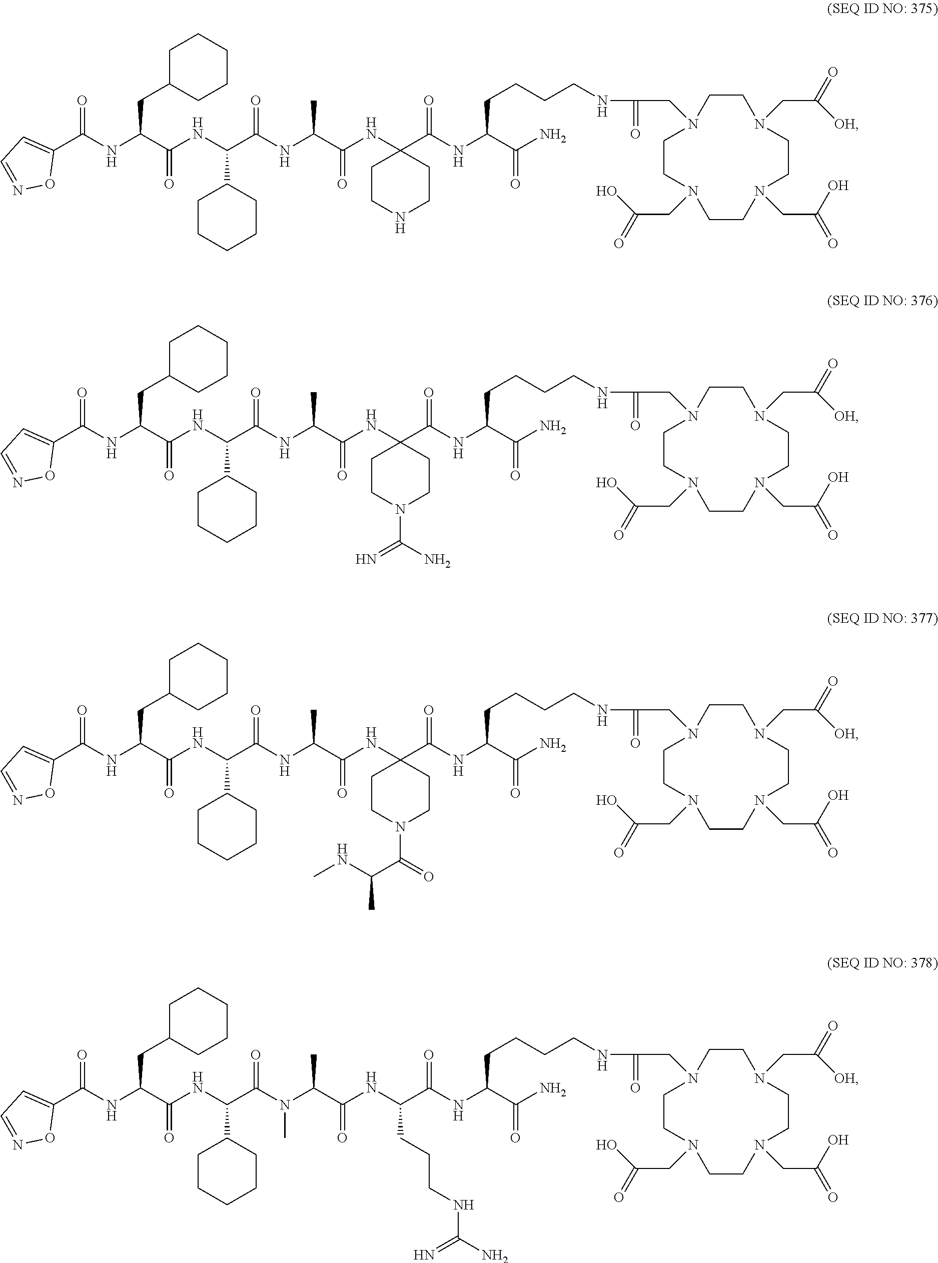

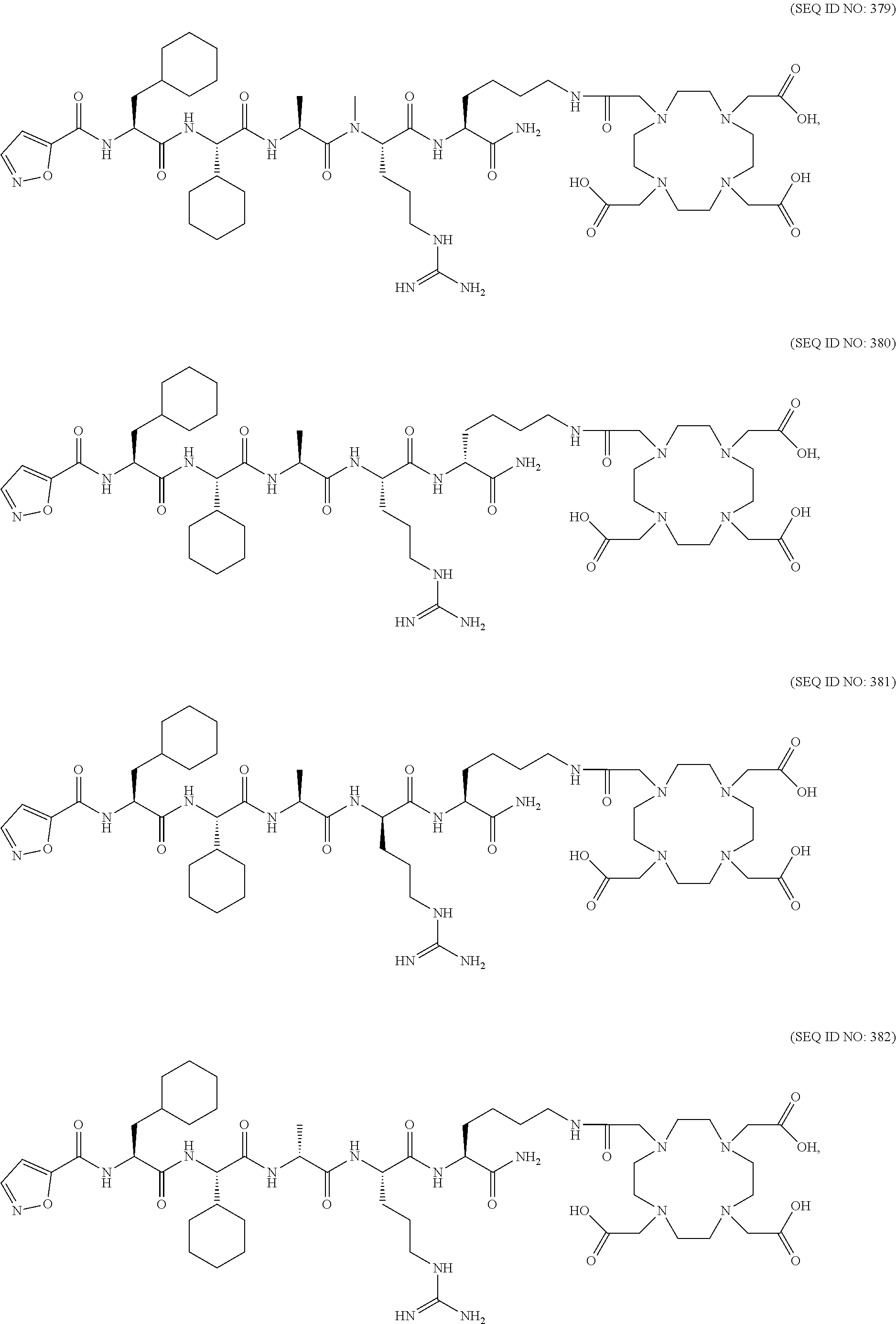

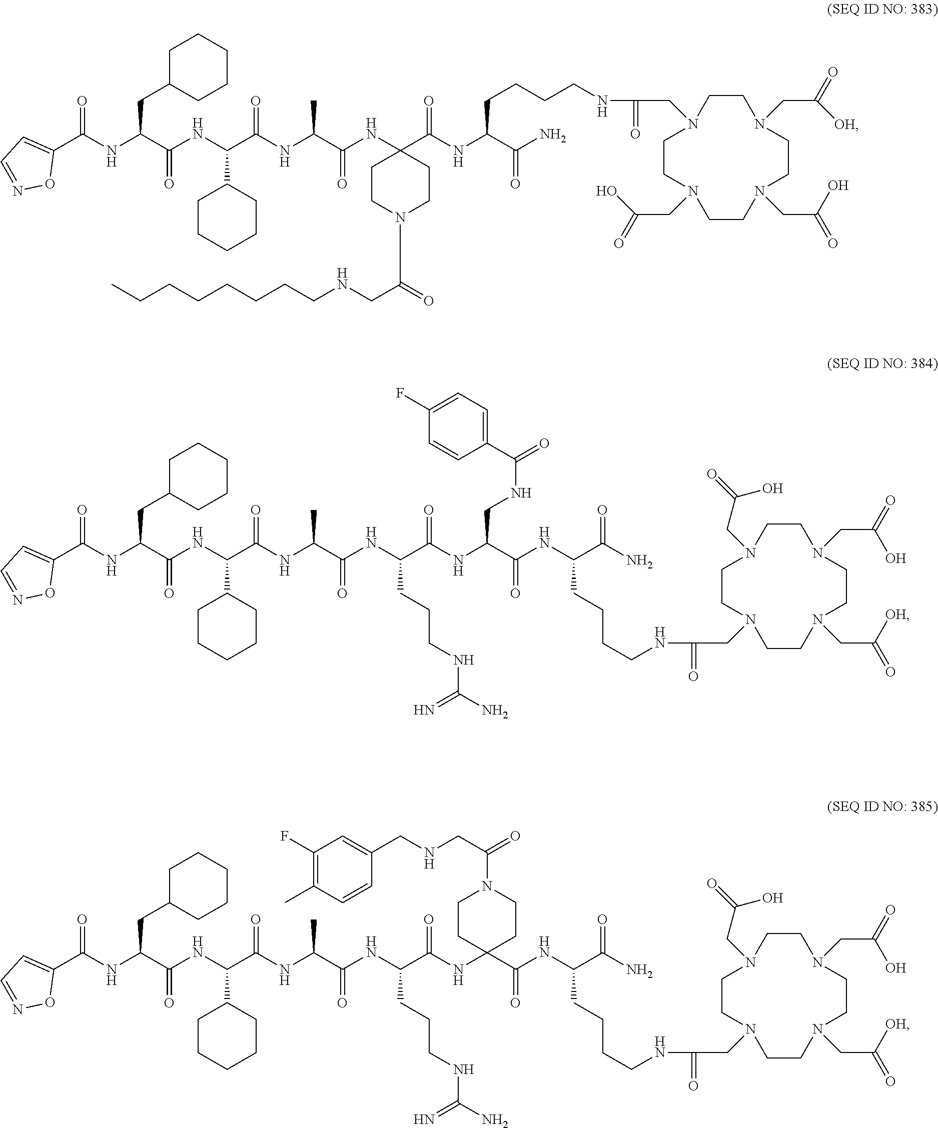

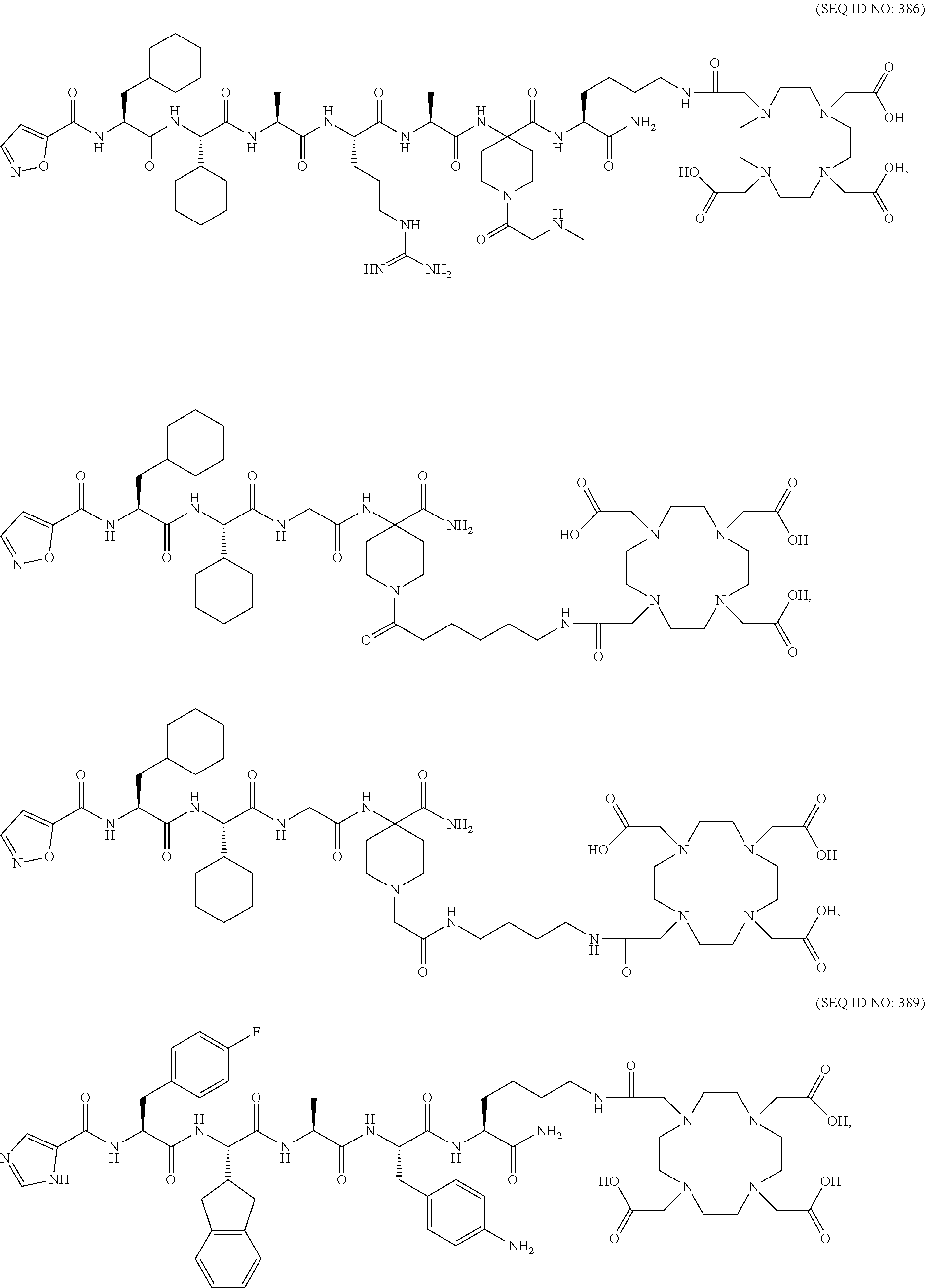

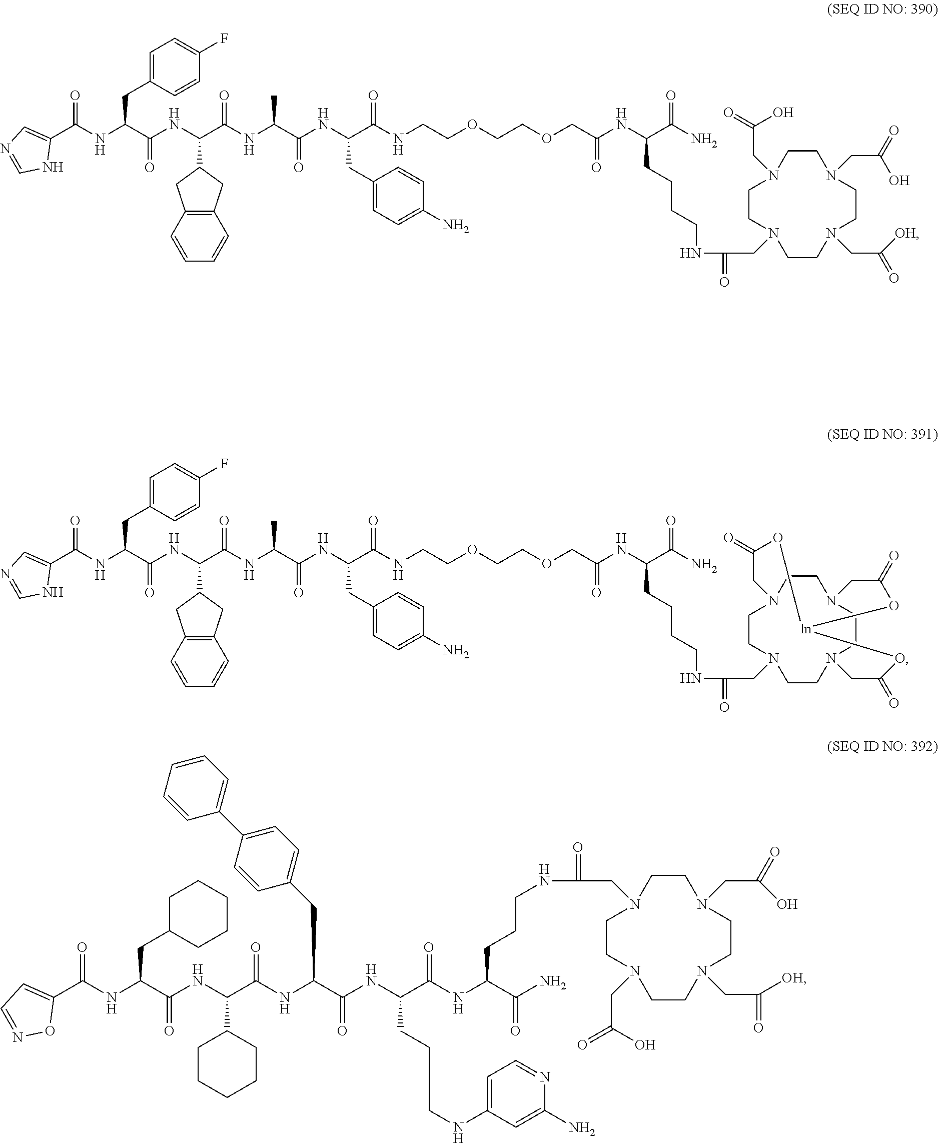

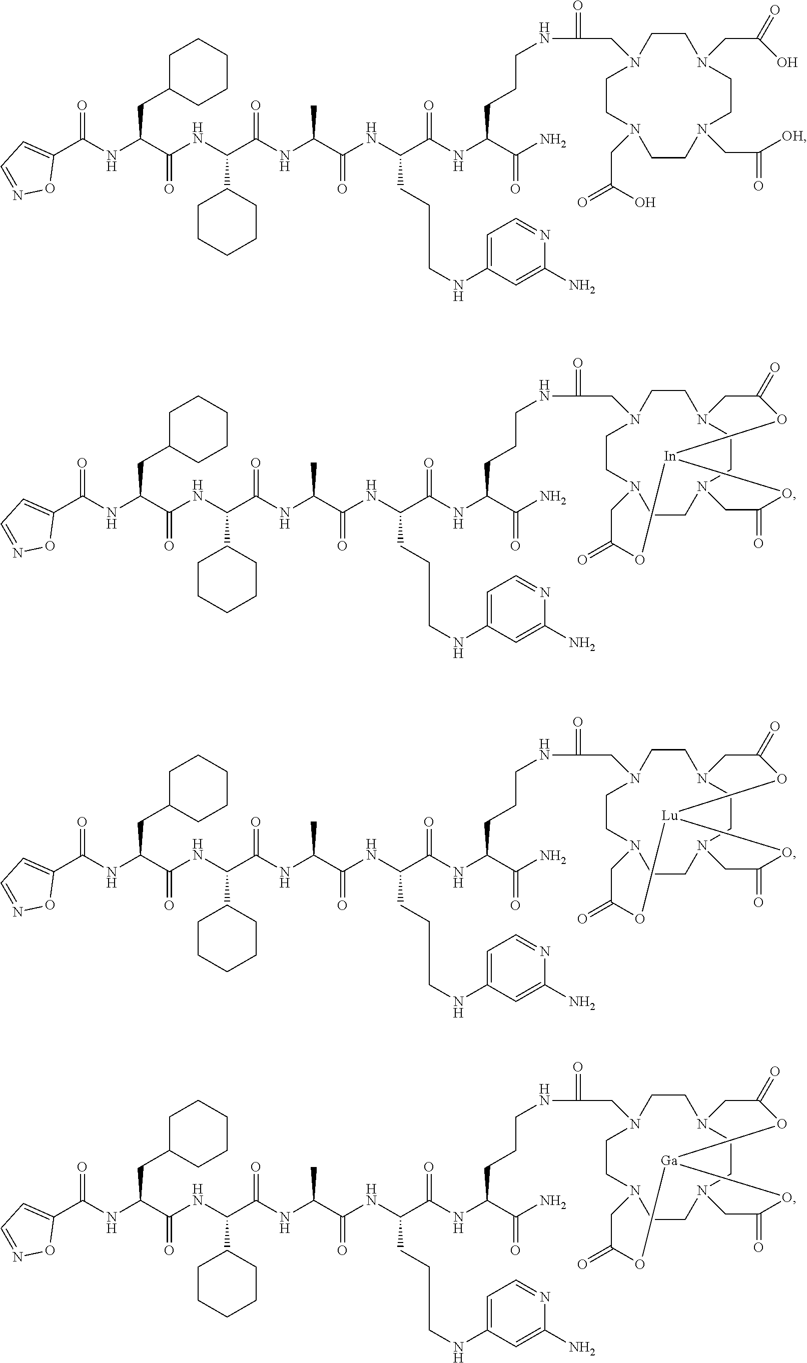

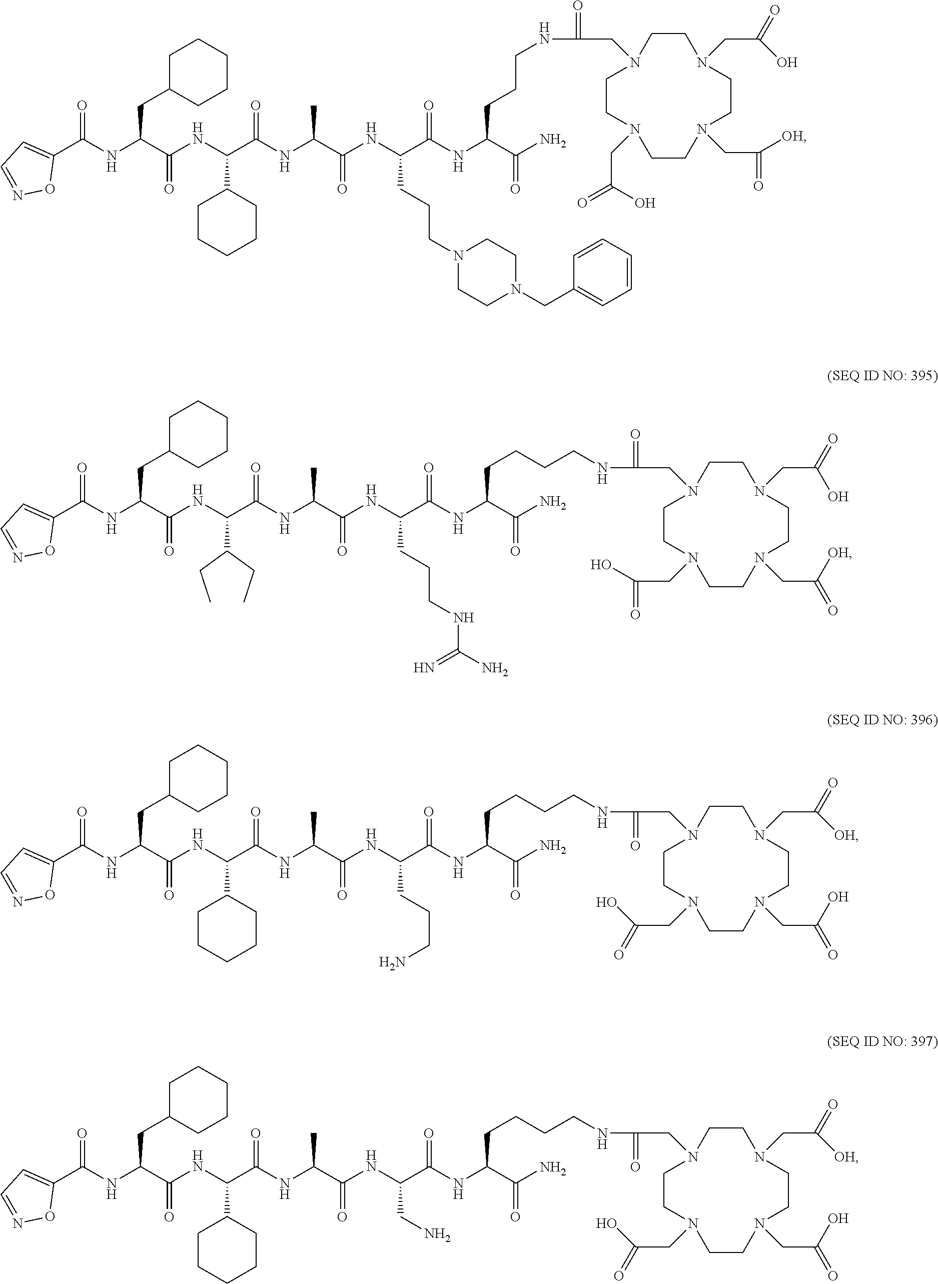

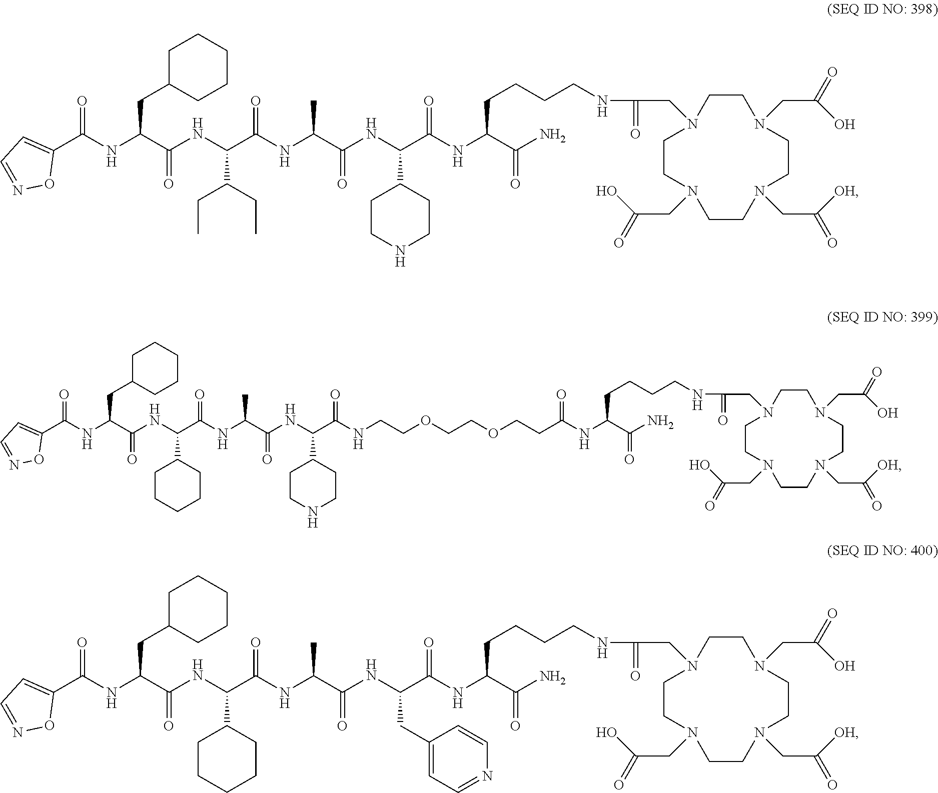 [SMILES: CCC(CC)[C@H](NC(=O)[C@H](CC1CCCCC1)NC(=O)c1ccno1)C(=O)N[C@@H](C)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1 ; C[C@H](NC(=O)[C@@H](NC(=O)[C@H](CC1CCCCC1)NC(=O)c1ccno1)C1CCCCC1)C(=O)N[C@H](C(=O)NCCOCCOCCC(=O)N[C@@H](CCCCNC(=O)CN1CCN(CC(=O)O)CCN(CC(=O)O)CCN(CC(=O)O)CC1)C(N)=O)C1CCNCC1 ; C[C@H](NC(=O)[C@@H](NC(=O)[C@H](CC1CCCCC1)NC(=O)c1ccno1)C1CCCCC1)C(=O)N[C@@H](Cc1ccncc1)C(=O)N[C@@H](CCCCNC(=O)CN1CCN(CC(=O)O)CCN(CC(=O)O)CCN(CC(=O)O)CC1)C(N)=O]

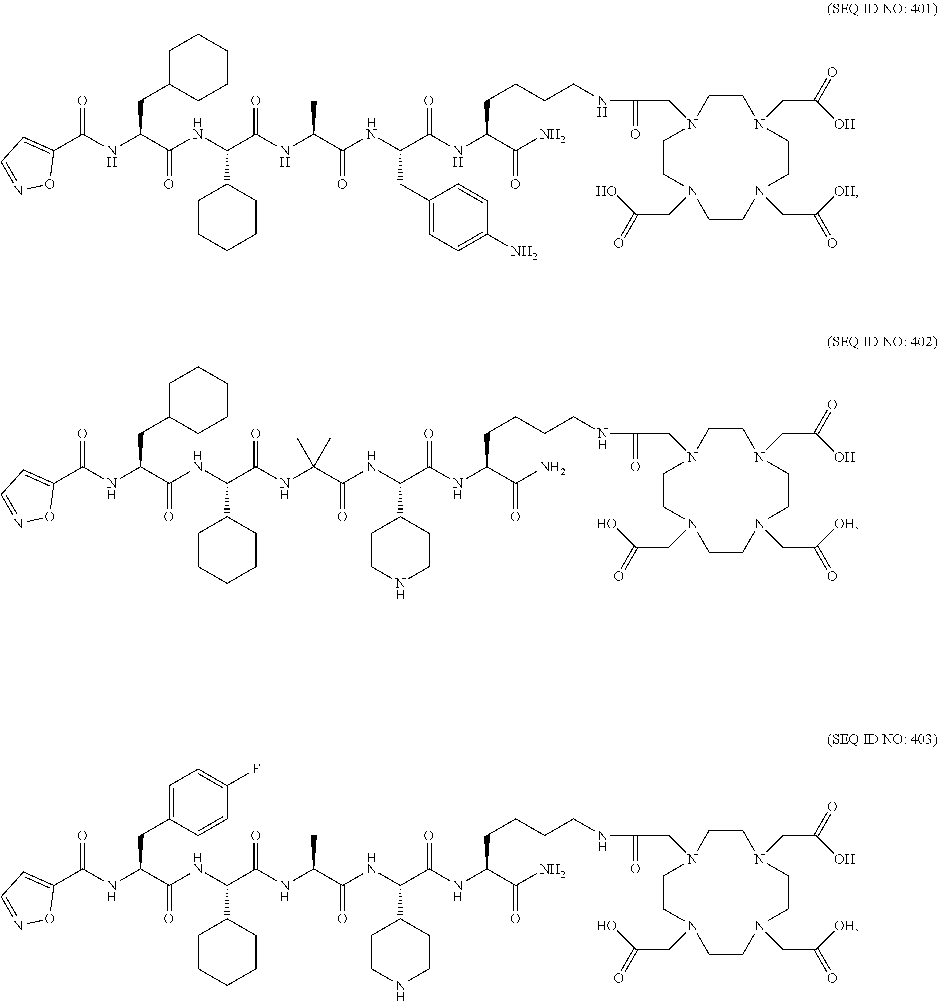 [SMILES: C[C@H](NC(=O)[C@@H](NC(=O)[C@H](CC1CCCCC1)NC(=O)c1ccno1)C1CCCCC1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(N)=O ; CC(C)(NC(=O)[C@@H](NC(=O)[C@H](CC1CCCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1 ; C[C@H](NC(=O)[C@@H](NC(=O)[C@H](Cc1ccc(F)cc1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

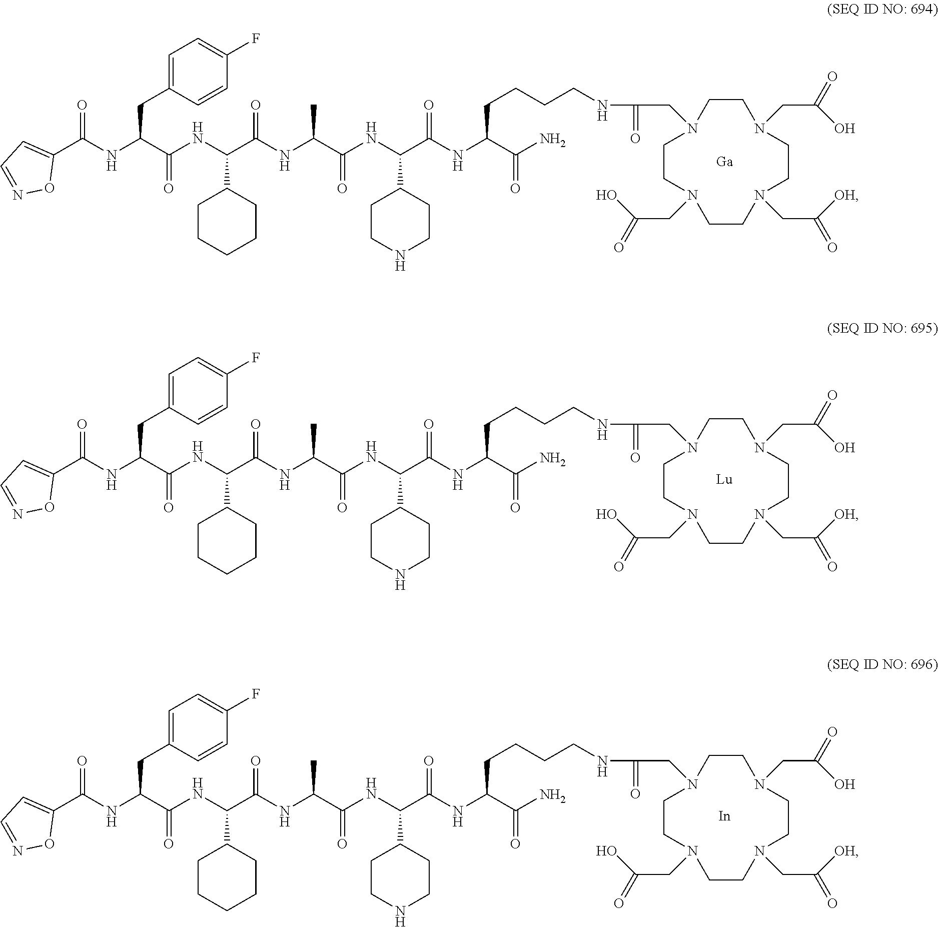

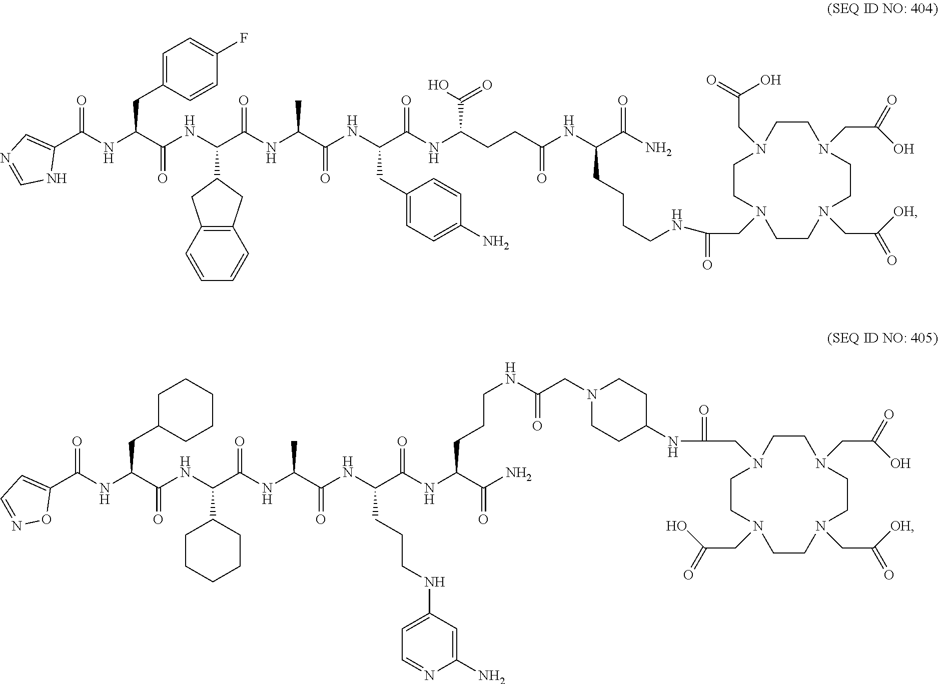 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@@H](Cc1ccc(N)cc1)C(=O)N[C@@H](CCC(=O)N[C@H](CCCCNC(=O)CN1CCN(CC(=O)O)CCN(CC(=O)O)CCN(CC(=O)O)CC1)C(N)=O)C(=O)O ; C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N[C@@H](CCCNC(=O)CN1CCC(NC(=O)CN2CCN(CC(=O)O)CCN(CC(=O)O)CCN(CC(=O)O)CC2)CC1)C(N)=O]

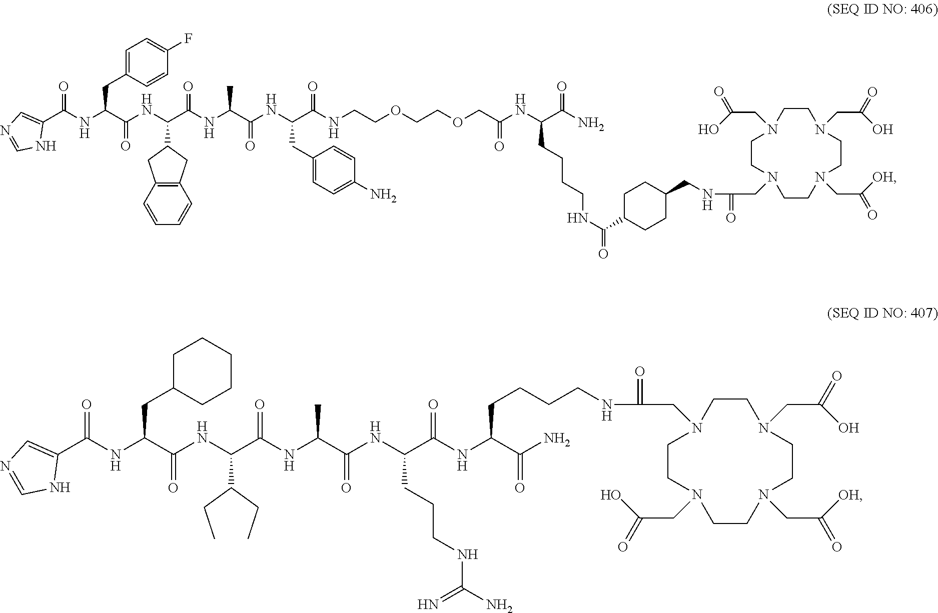

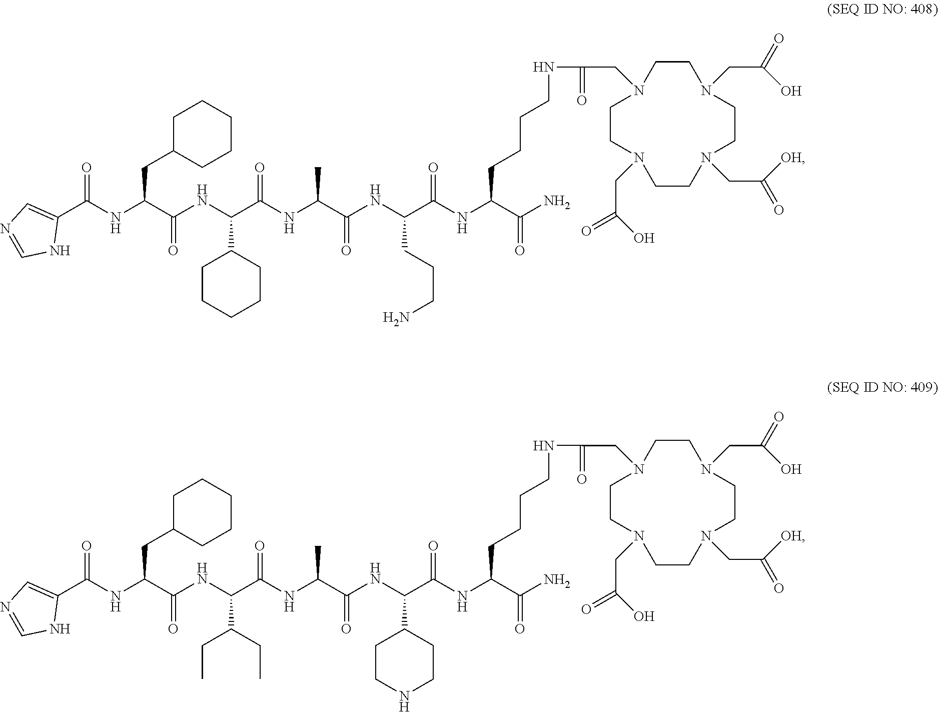

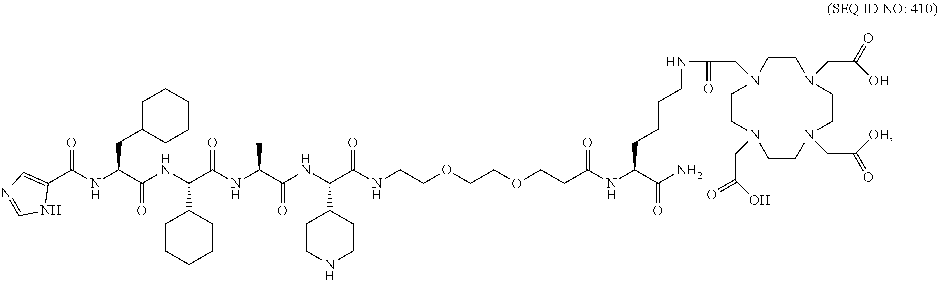 [SMILES: C[C@H](NC(=O)[C@@H](NC(=O)[C@H](CC1CCCCC1)NC(=O)c1cnc[nH]1)C1CCCCC1)C(=O)N[C@H](C(=O)NCCOCCOCCC(=O)N[C@@H](CCCCNC(=O)CN1CCN(CC(=O)O)CCN(CC(=O)O)CCN(CC(=O)O)CC1)C(N)=O)C1CCNCC1]

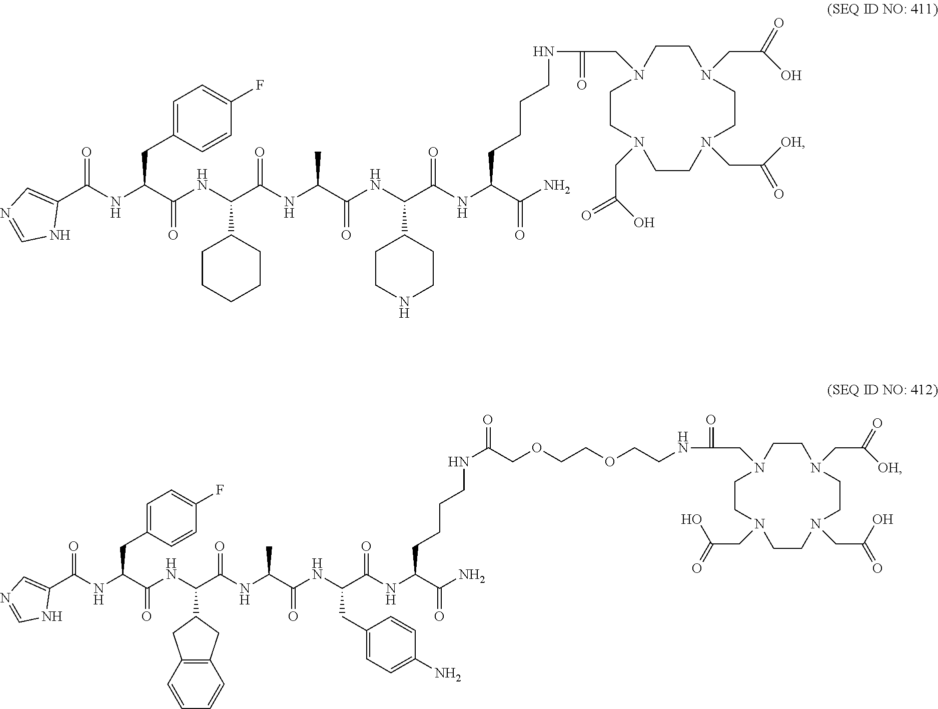 [SMILES: C[C@H](NC(=O)[C@@H](NC(=O)[C@H](Cc1ccc(F)cc1)NC(=O)c1cnc[nH]1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1 ; C[C@H](NC(=O)[C@@H](NC(=O)[C@H](Cc1ccc(F)cc1)NC(=O)c1cnc[nH]1)C1Cc2ccccc2C1)C(=O)N[C@@H](Cc1ccc(N)cc1)C(=O)N[C@@H](CCCCNC(=O)COCCOCCNC(=O)CN1CCN(CC(=O)O)CCN(CC(=O)O)CCN(CC(=O)O)CC1)C(N)=O]

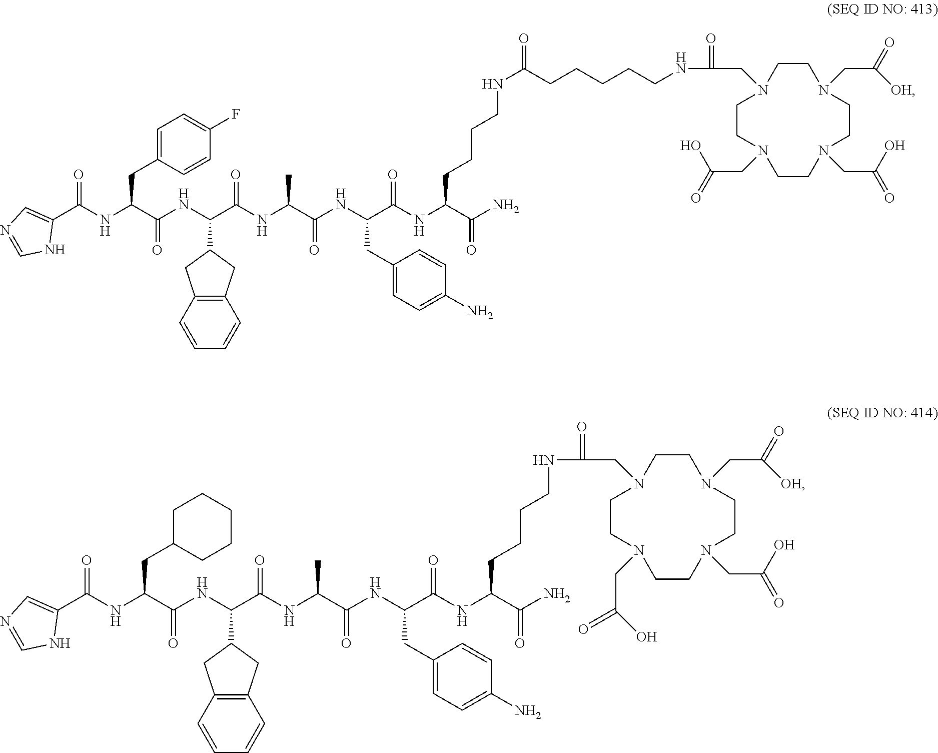 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@@H](Cc1ccc(N)cc1)C(=O)N[C@@H](CCCCNC(=O)CCCCCNC(=O)CN1CCN(CC(=O)O)CCN(CC(=O)O)CCN(CC(=O)O)CC1)C(N)=O ; C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(N)=O]

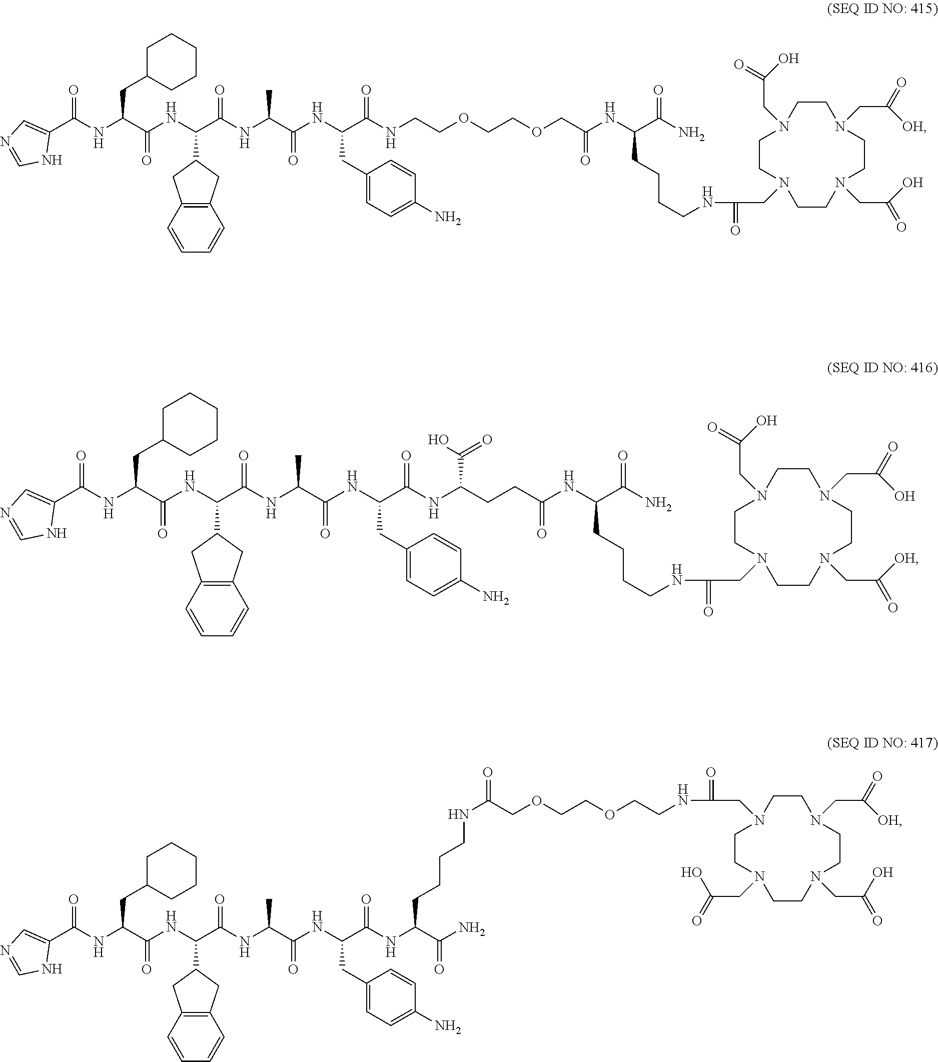

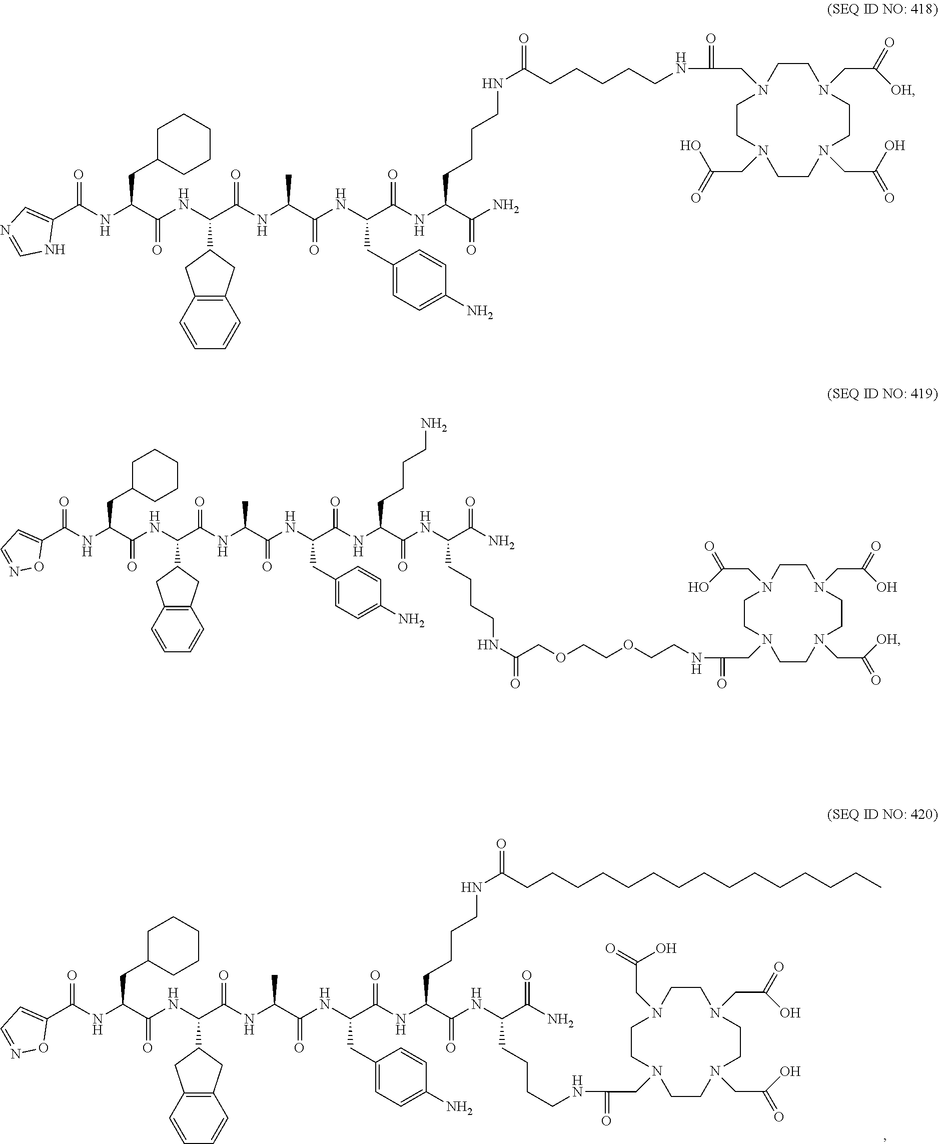

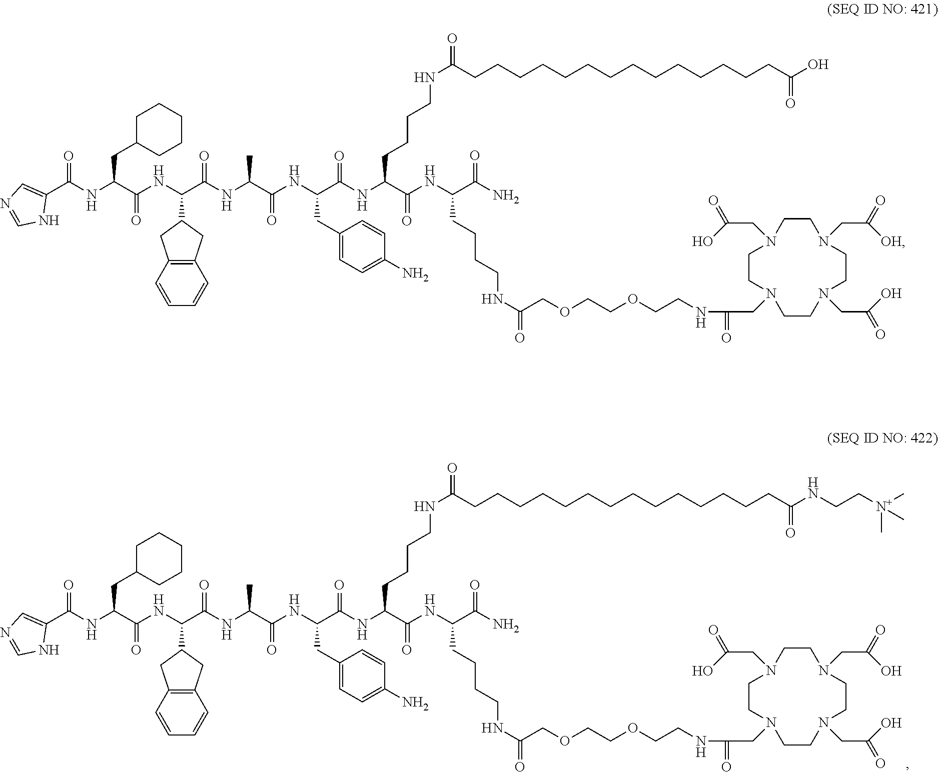 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCCNC(=O)CCCCCCCCCCCCCCC(=O)O)C(=O)N[C@@H](CCCCNC(=O)COCCOCCNC(=O)CN1CCN(CC(=O)O)CCN(CC(=O)O)CCN(CC(=O)O)CC1)C(N)=O ; C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCCNC(=O)CCCCCCCCCCCCCCC(=O)NCC[N+](C)(C)C)C(=O)N[C@@H](CCCCNC(=O)COCCOCCNC(=O)CN1CCN(CC(=O)O)CCN(CC(=O)O)CCN(CC(=O)O)CC1)C(N)=O]

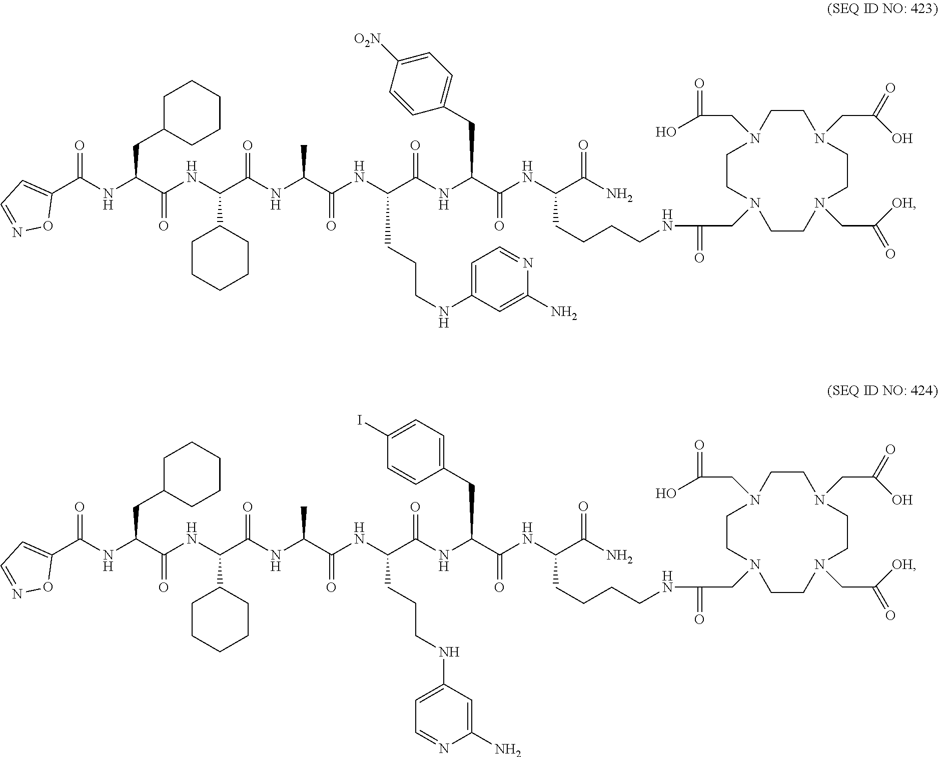 [SMILES: C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N[C@@H](Cc1ccc([N+](=O)[O-])cc1)C(=O)N[C@@H](CCCCNC(=O)CN1CCN(CC(=O)O)CCN(CC(=O)O)CCN(CC(=O)O)CC1)C(N)=O ; C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N[C@@H](Cc1ccc(I)cc1)C(=O)N[C@@H](CCCCNC(=O)CN1CCN(CC(=O)O)CCN(CC(=O)O)CCN(CC(=O)O)CC1)C(N)=O]

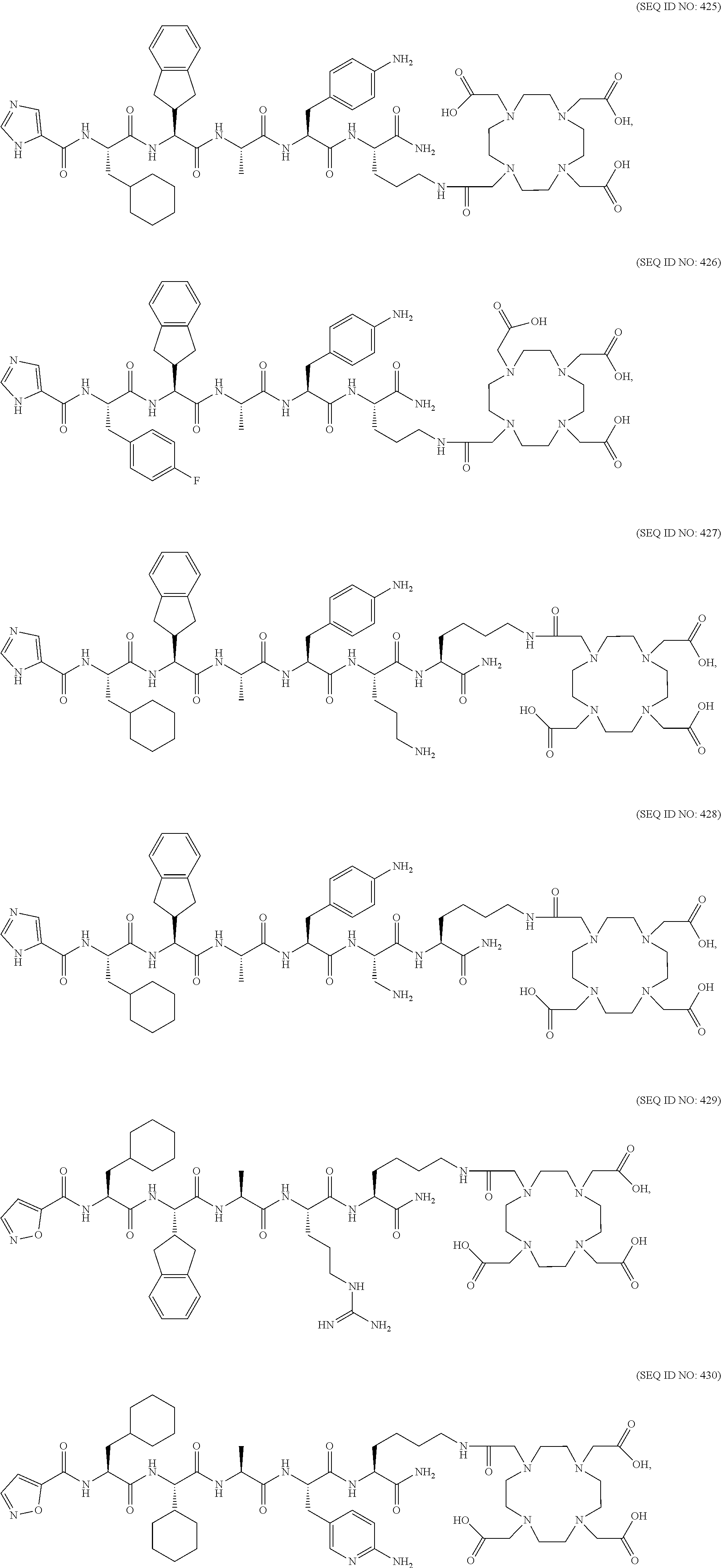

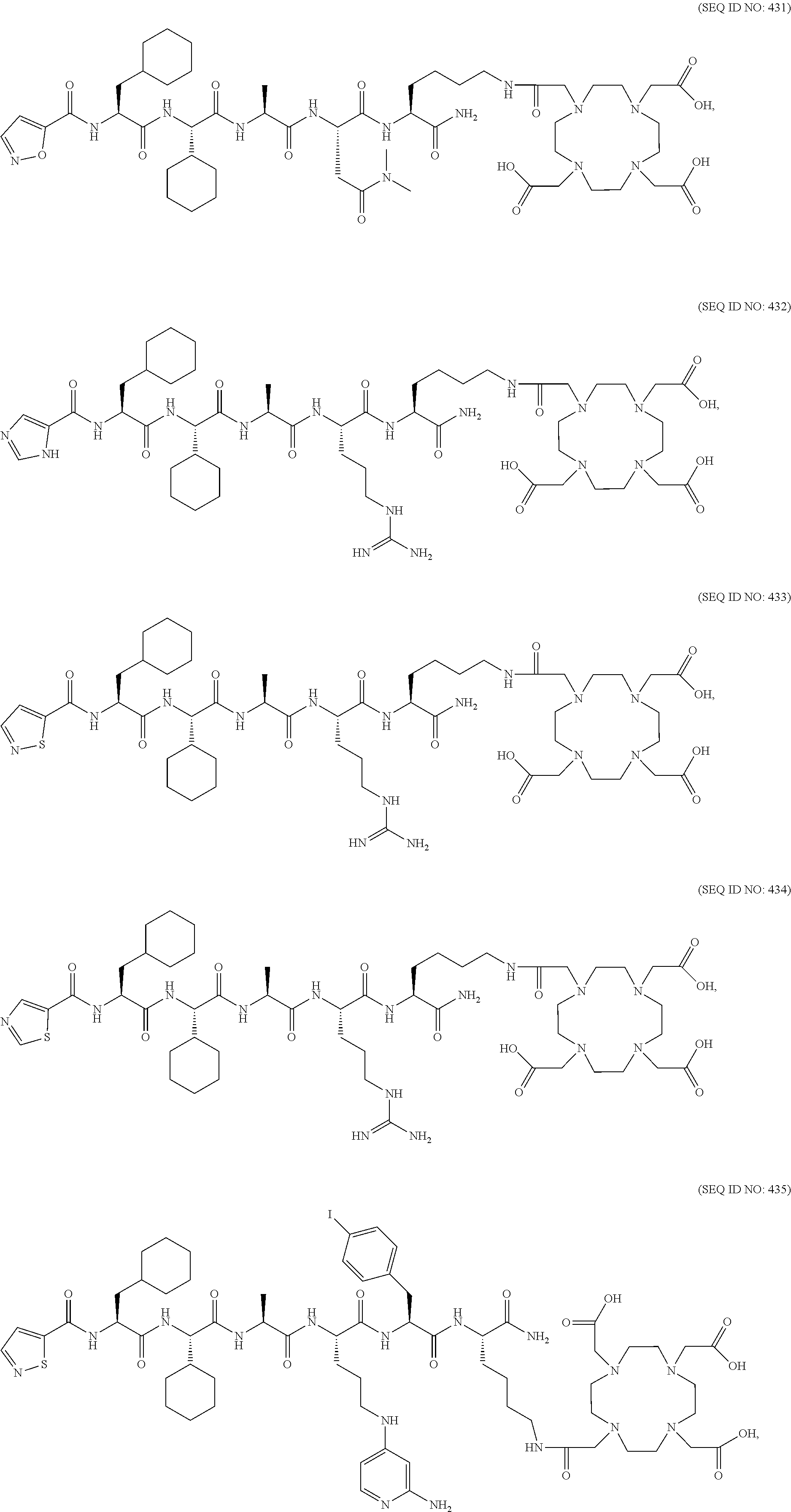

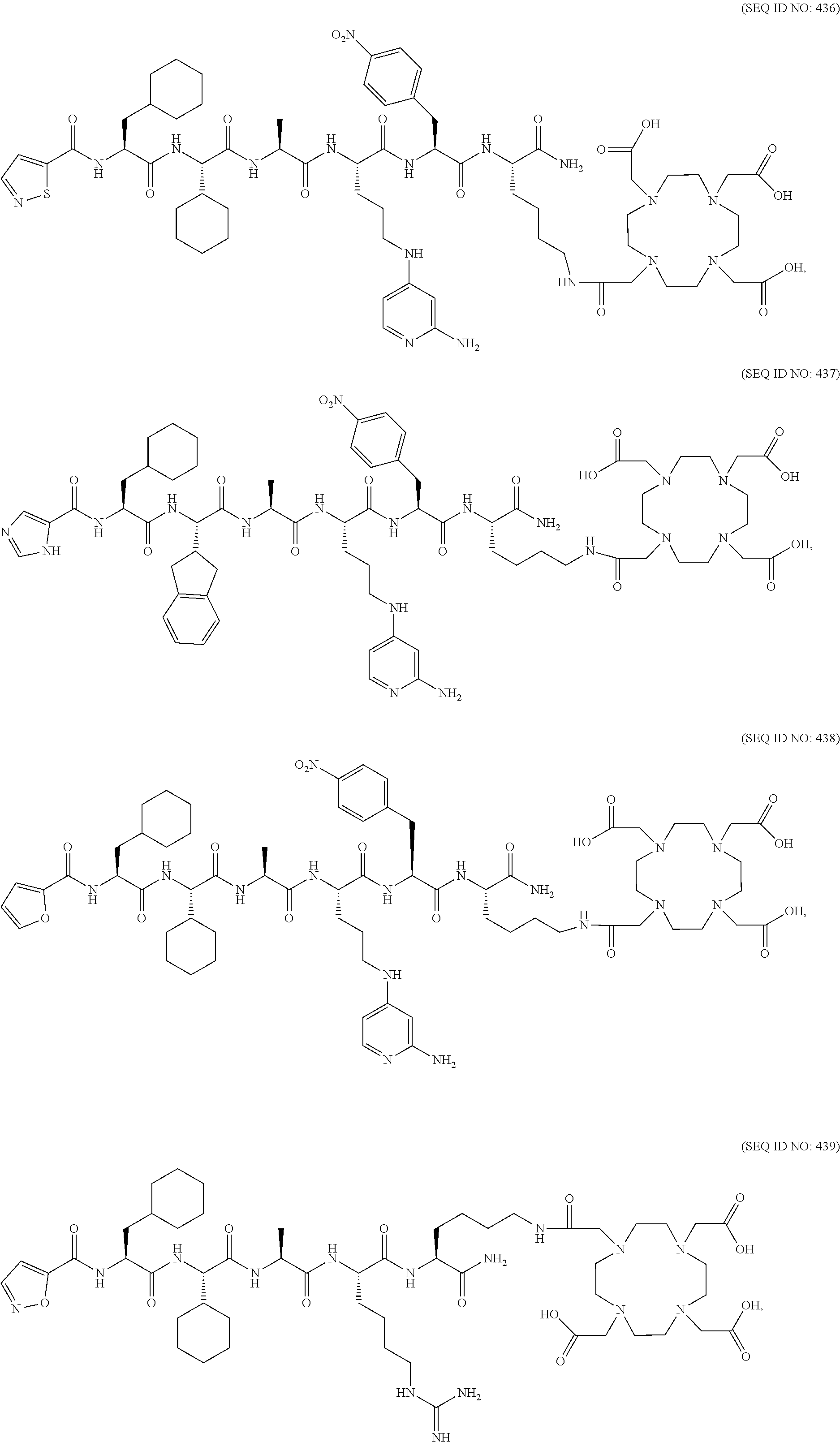

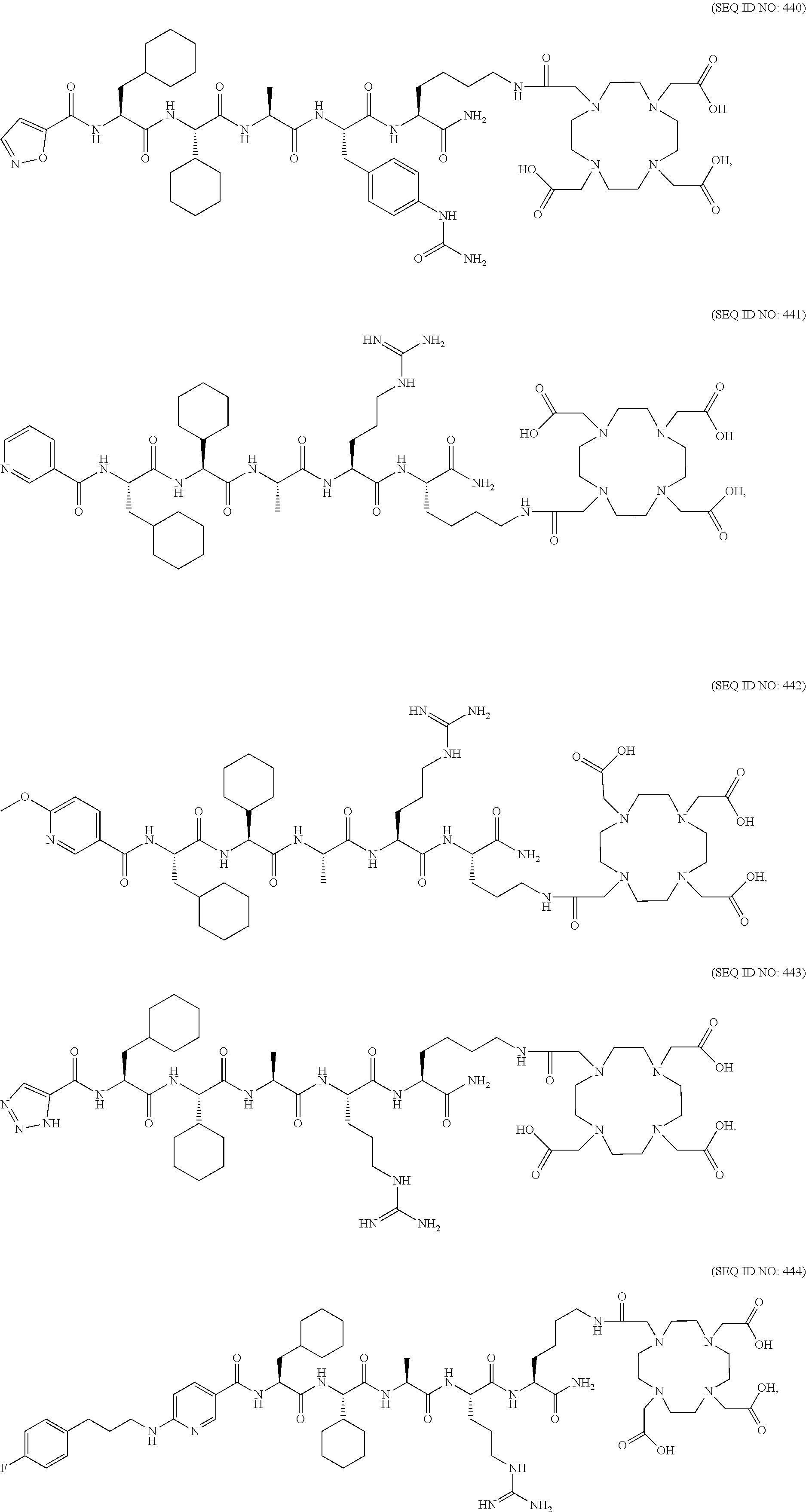

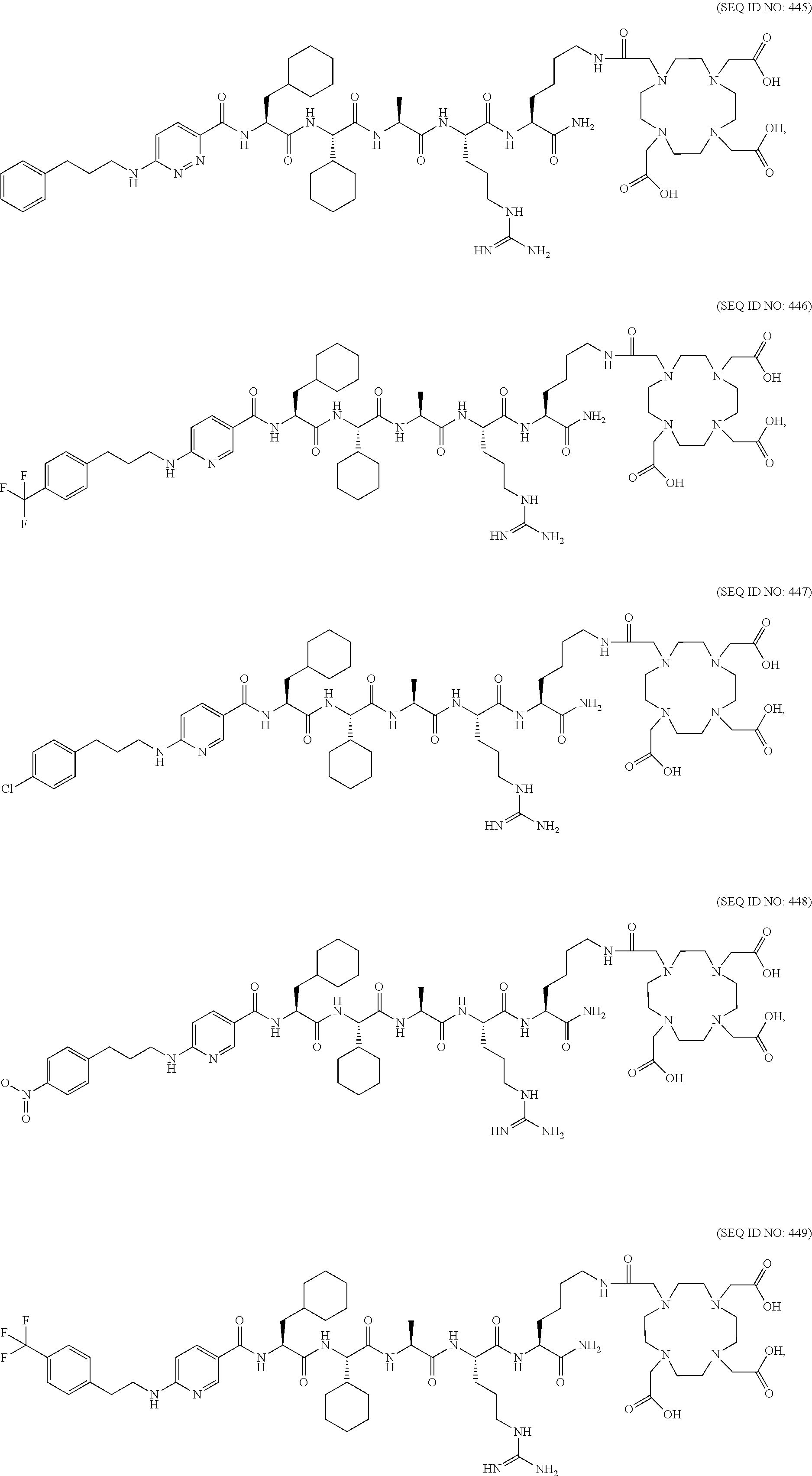

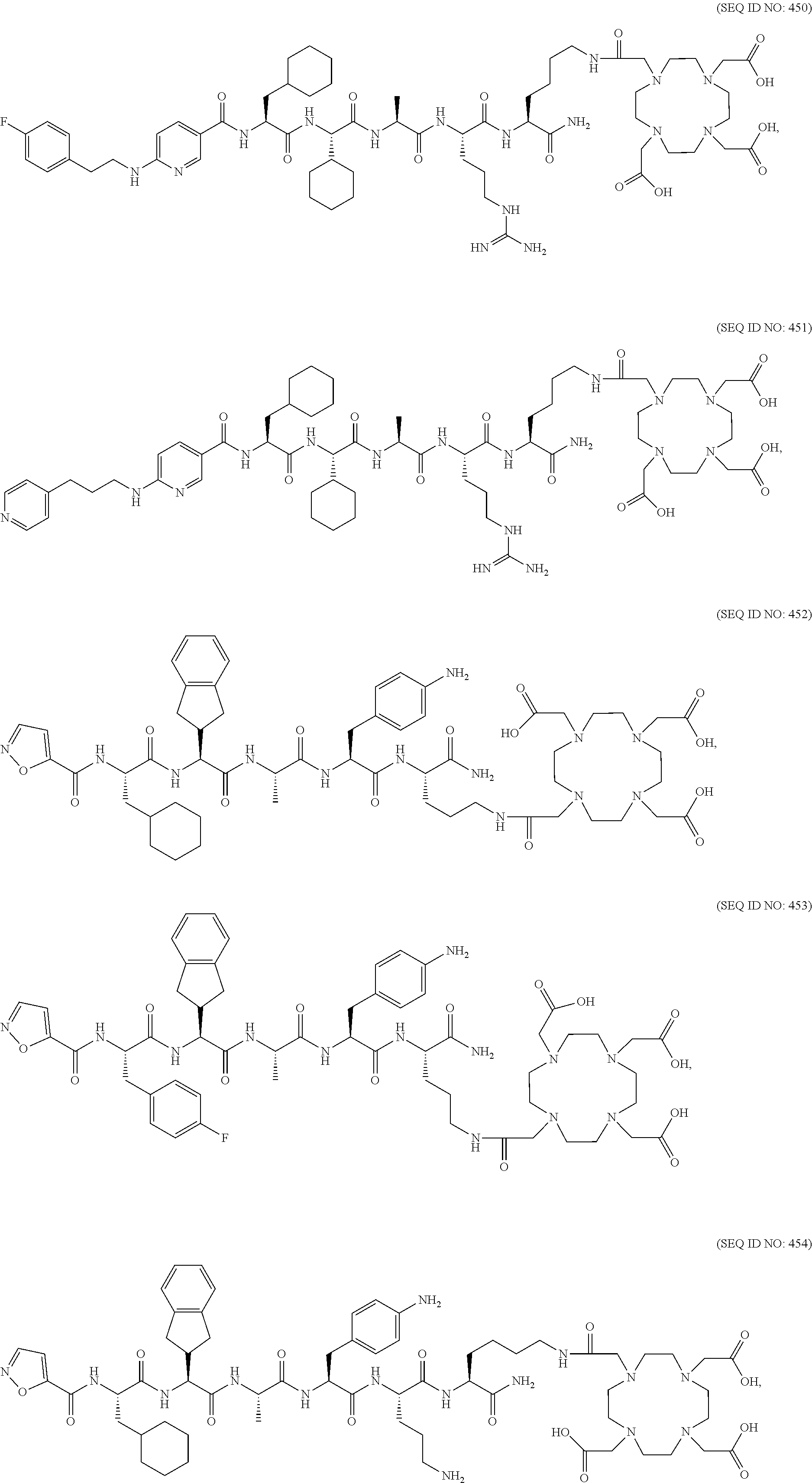

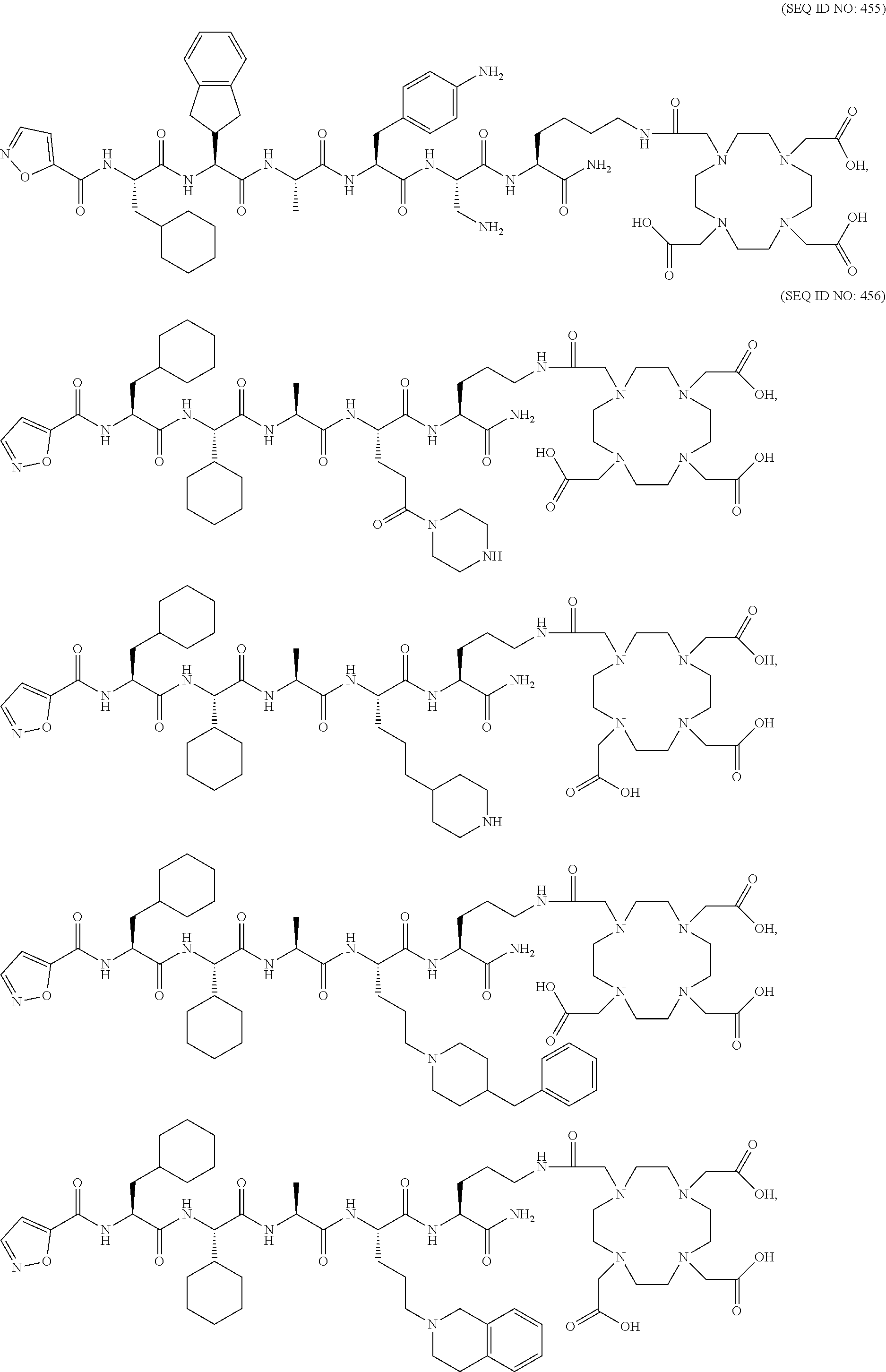

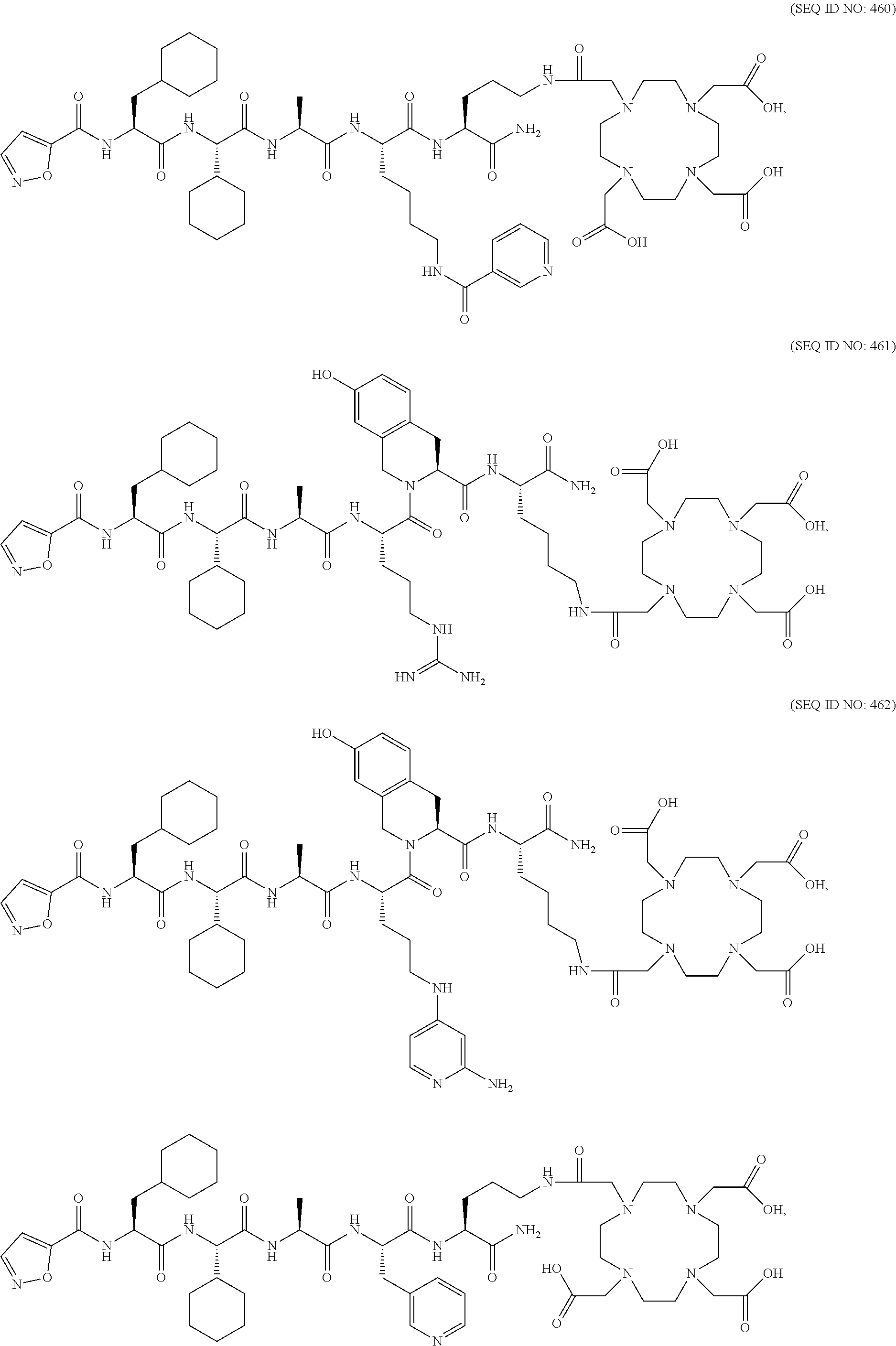

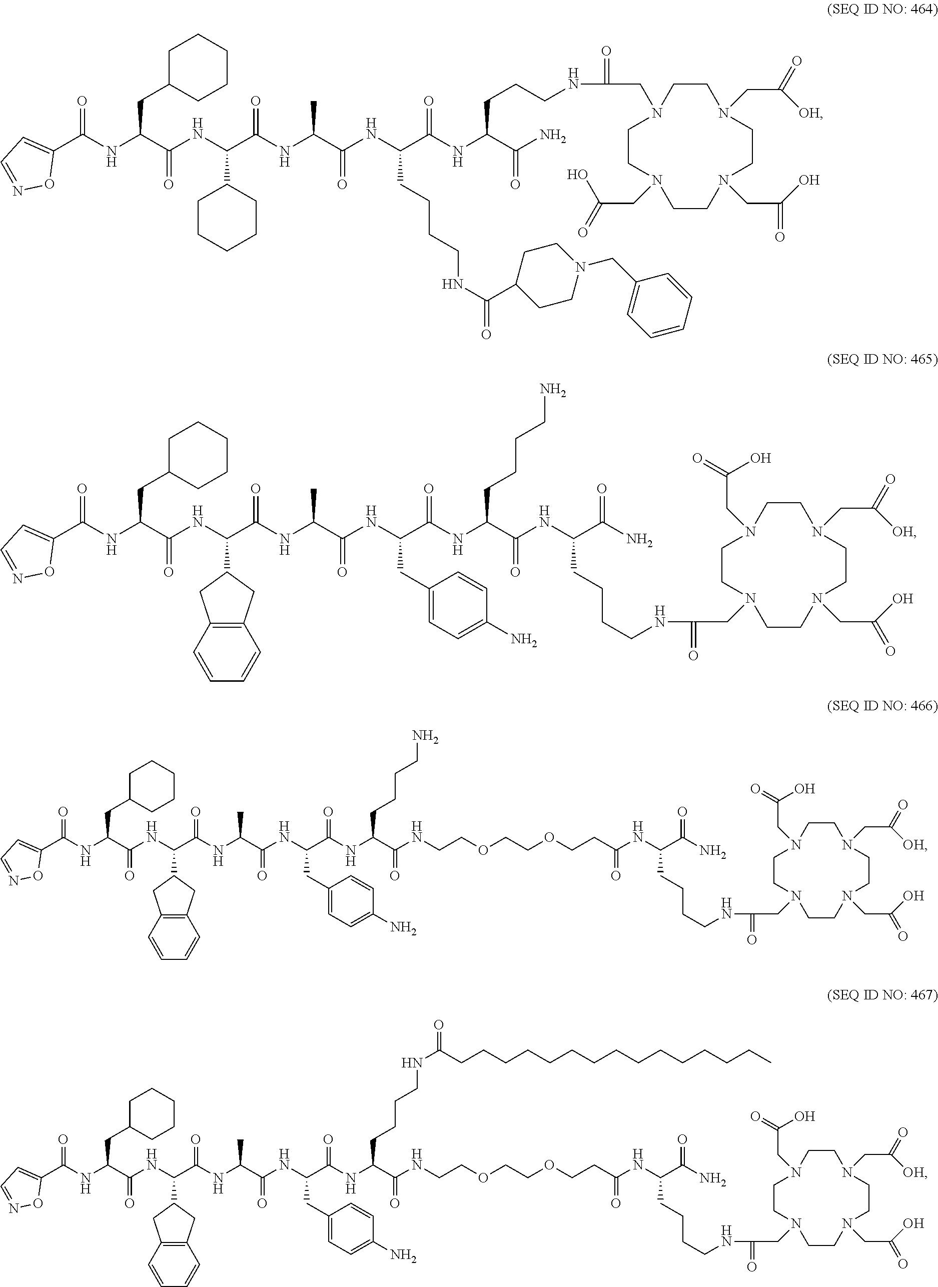

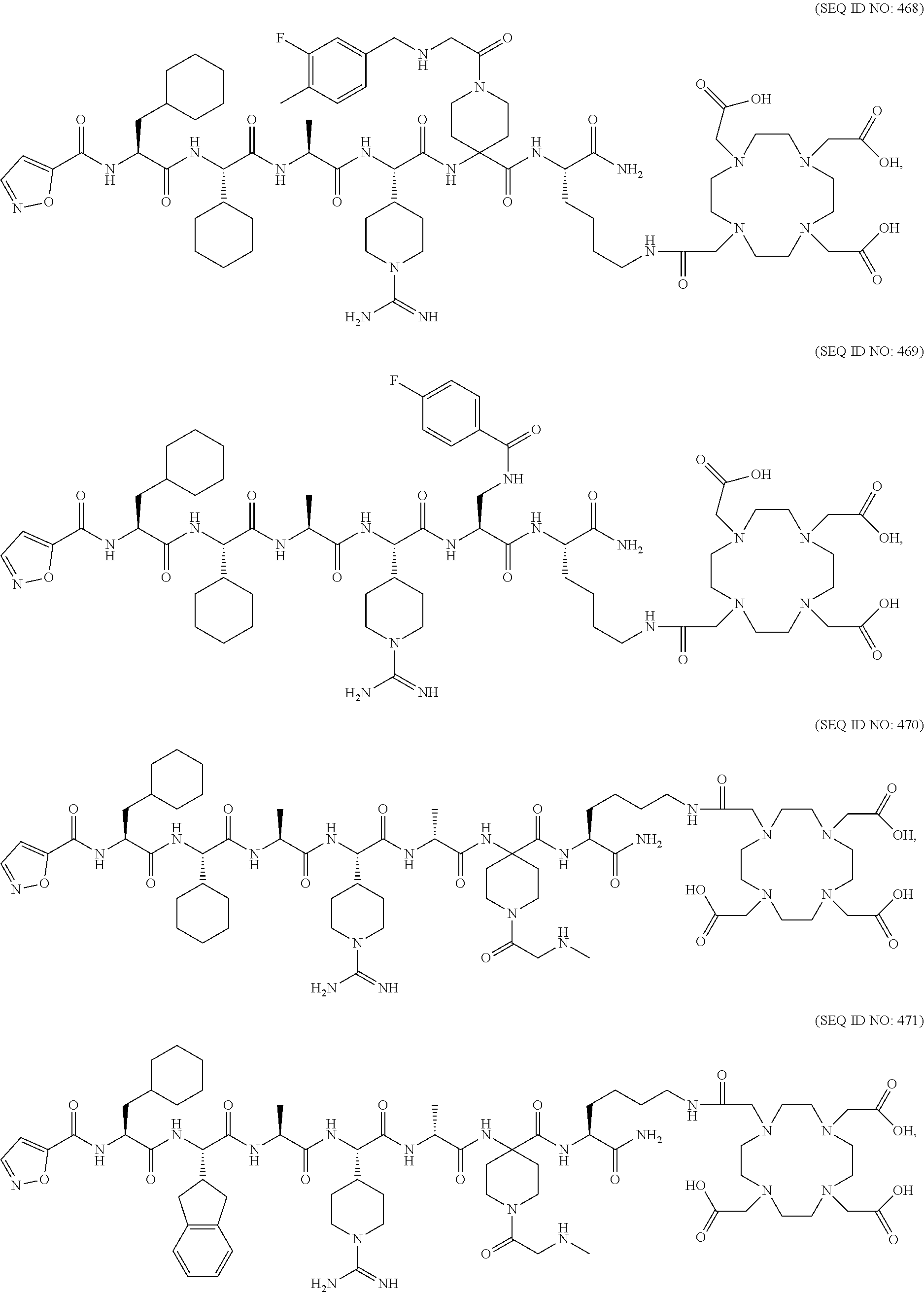

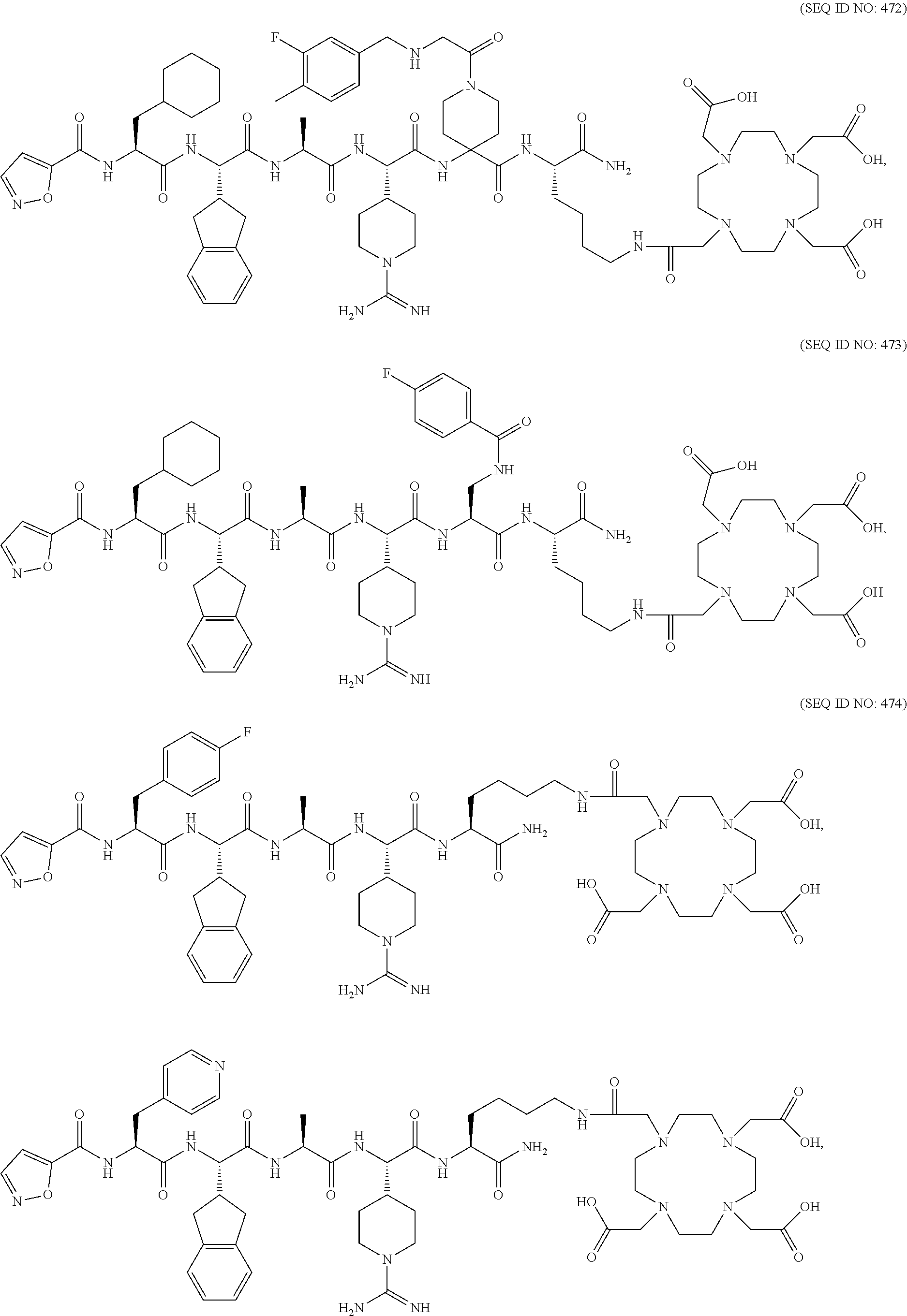

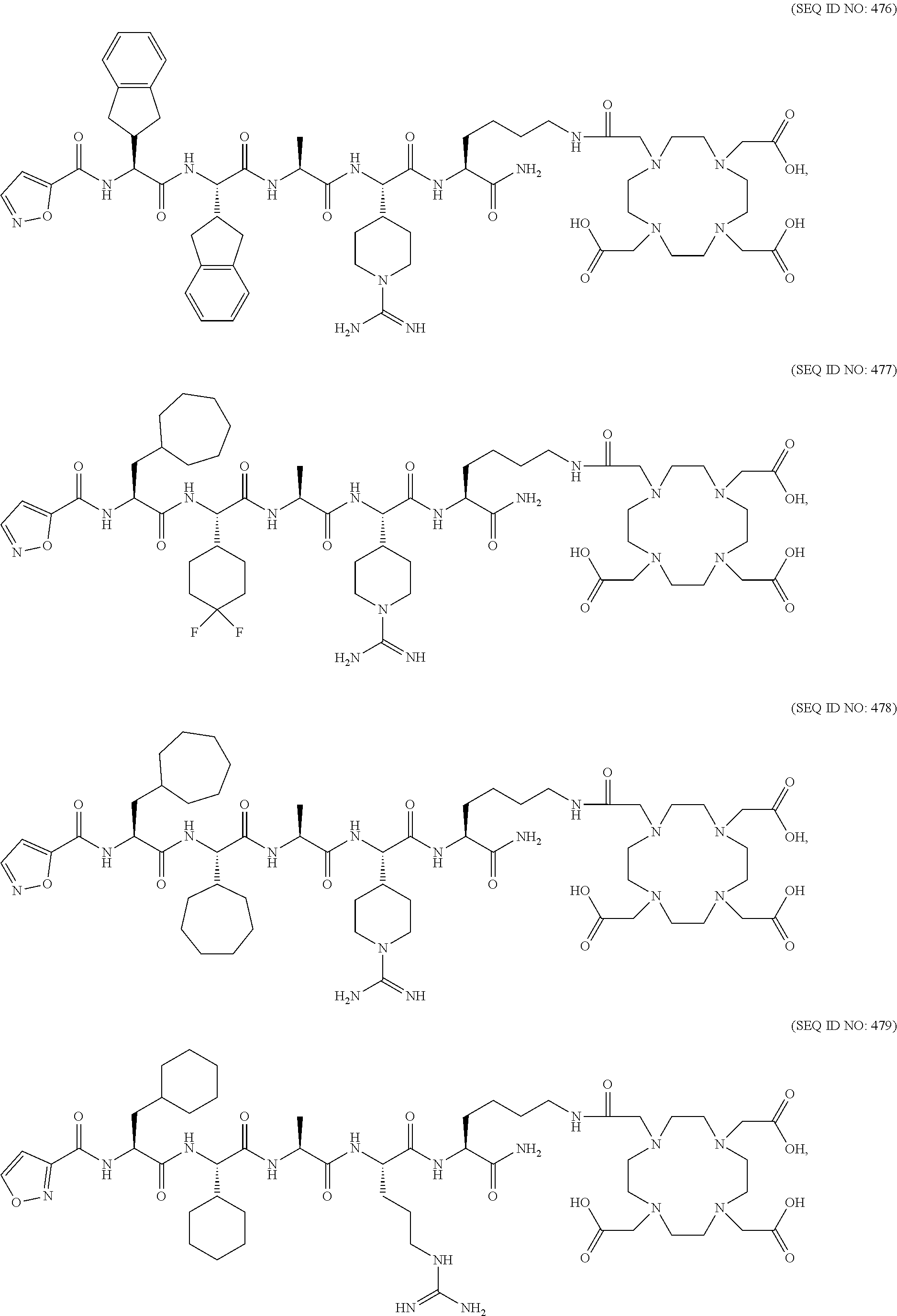

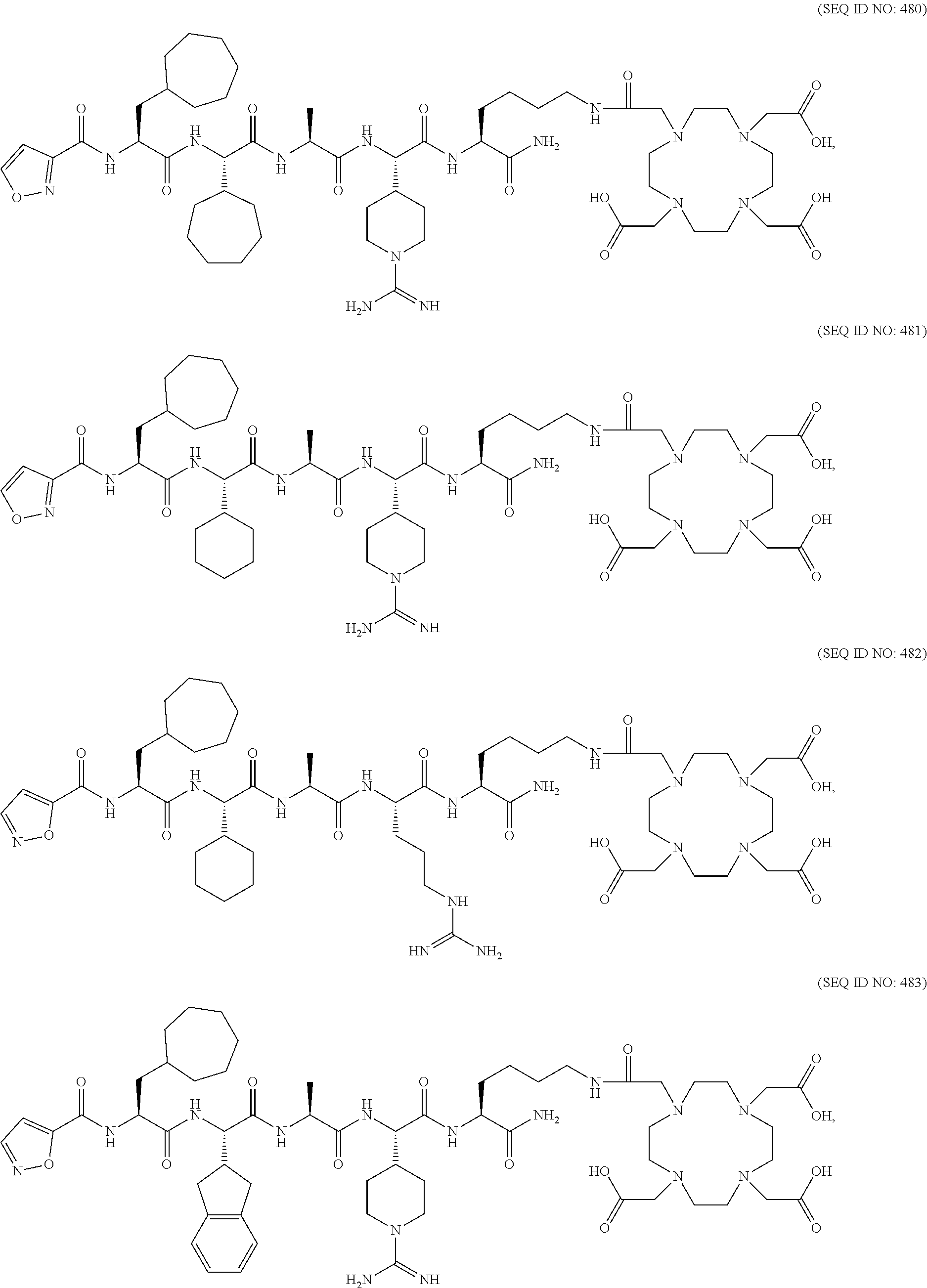

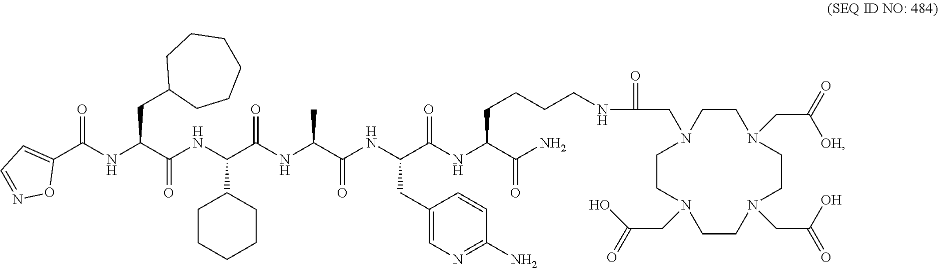 [SMILES: C[C@H](NC(=O)[C@@H](NC(=O)[C@H](CC1CCCCCC1)NC(=O)c1ccno1)C1CCCCC1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(N)=O]

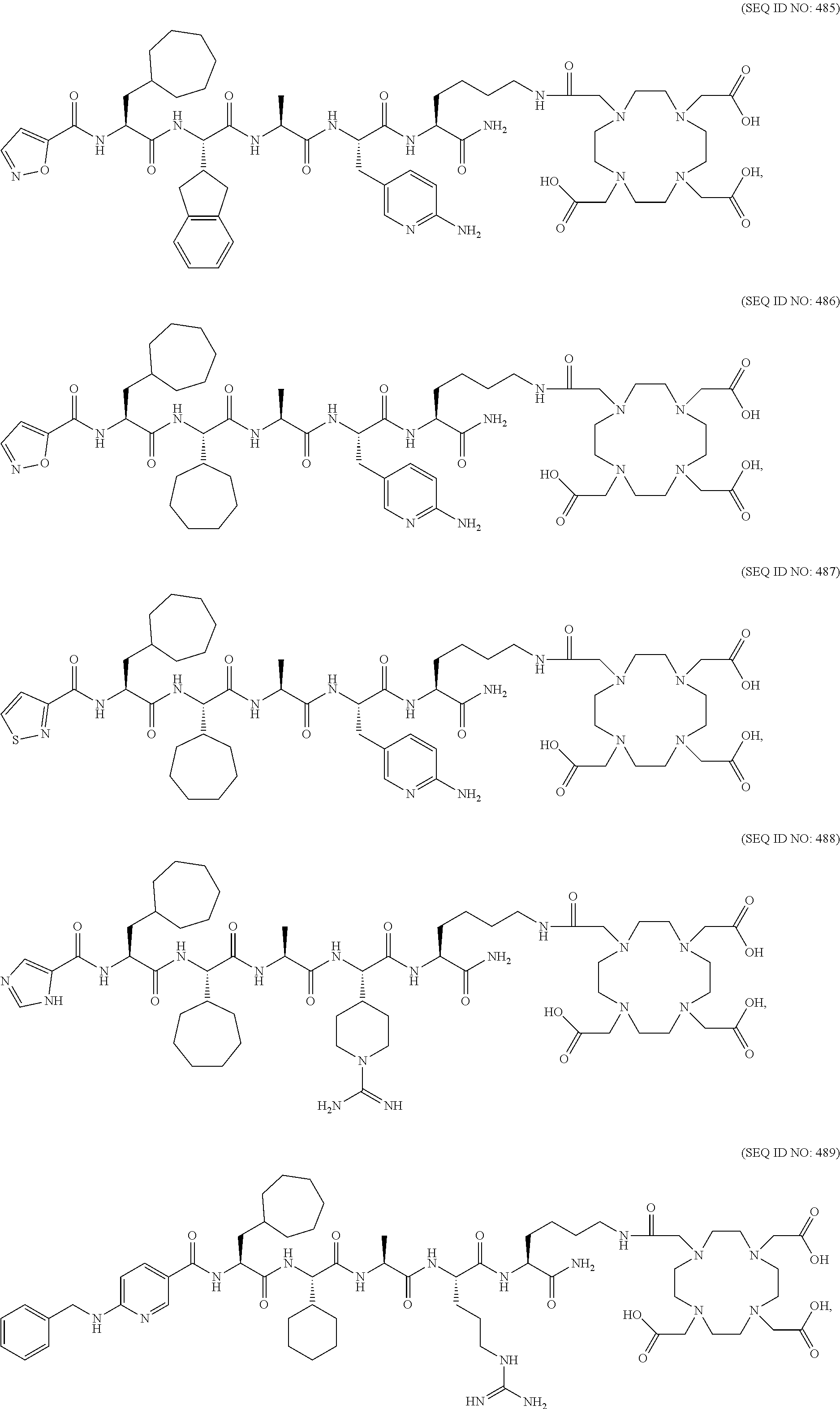

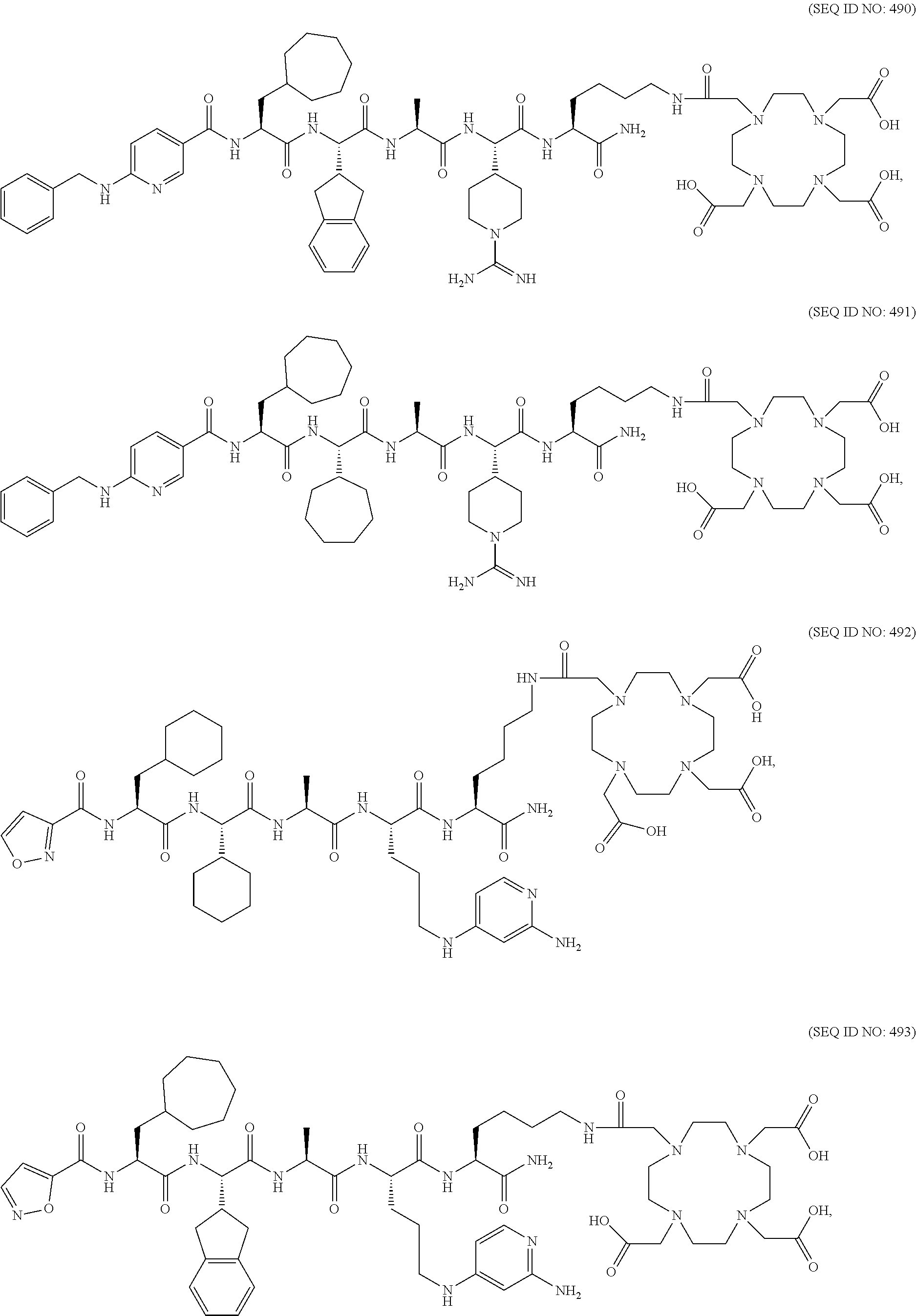

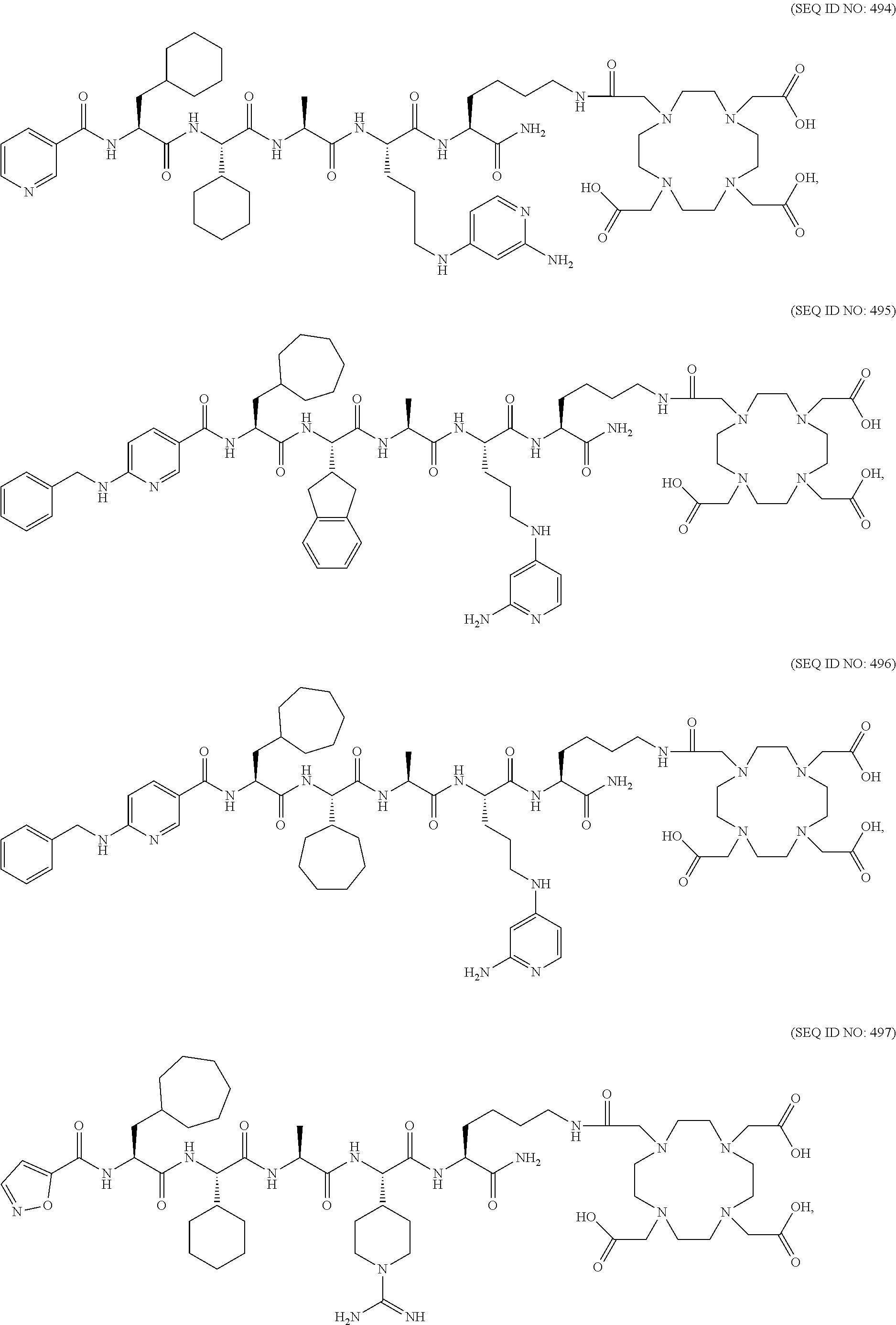

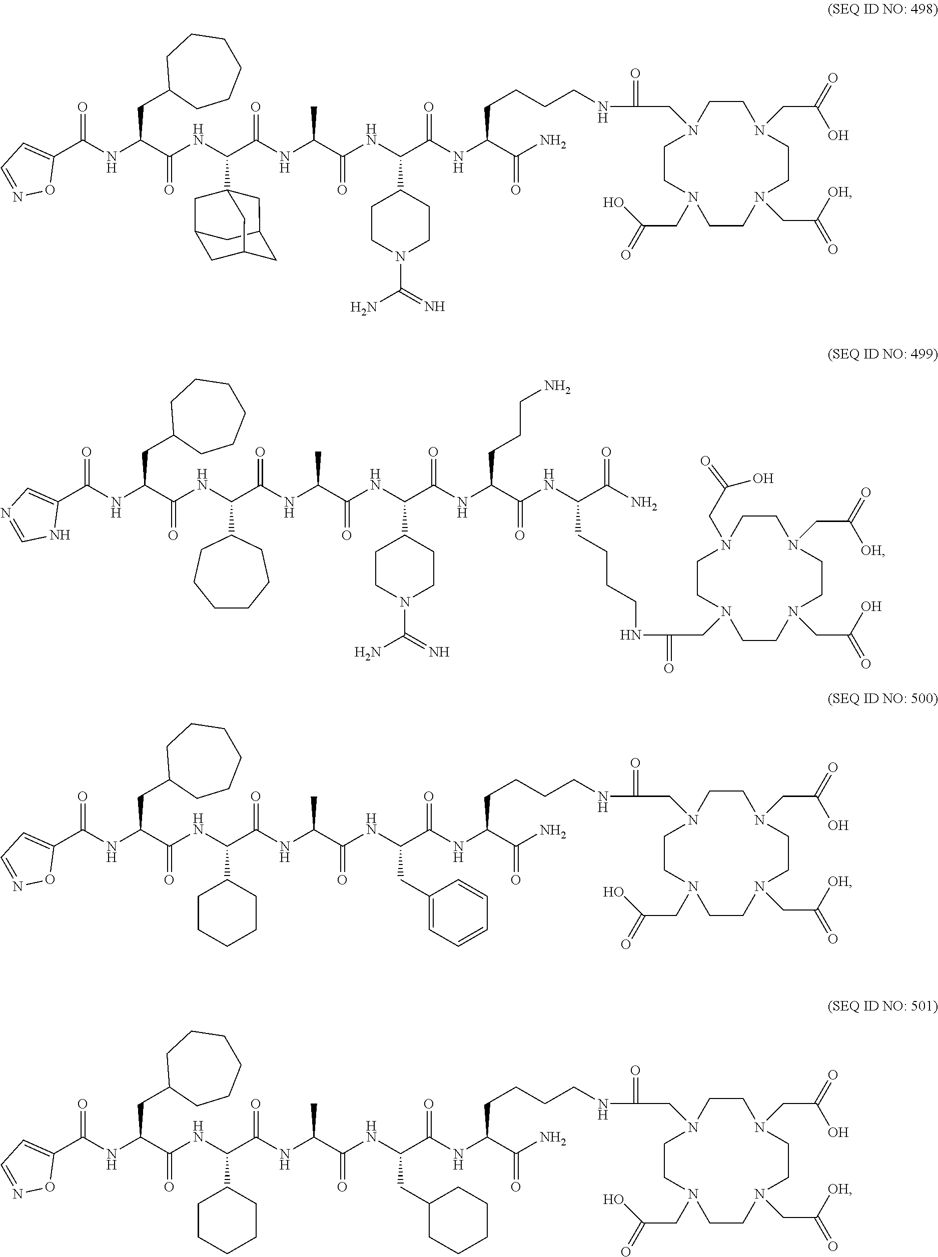

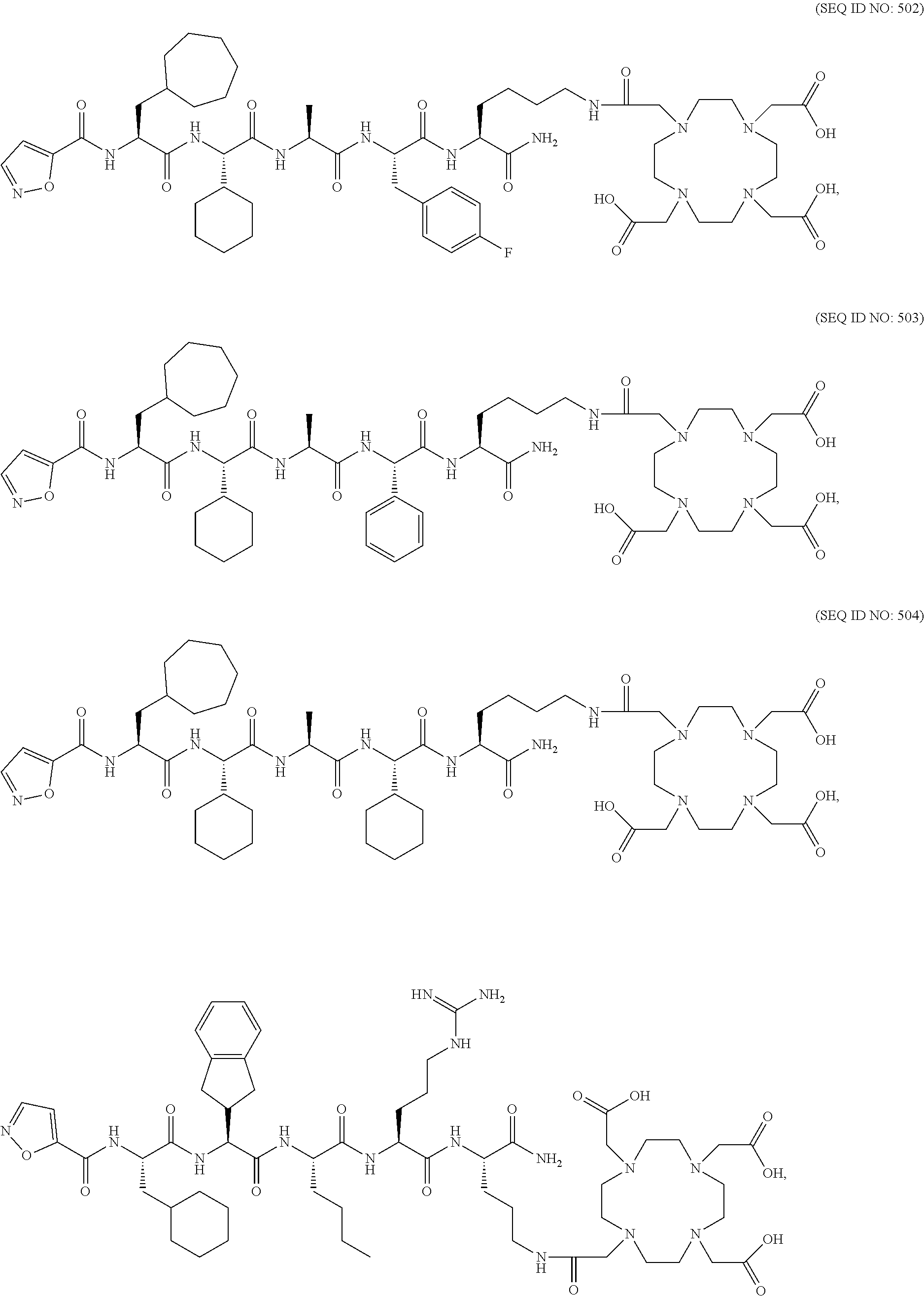

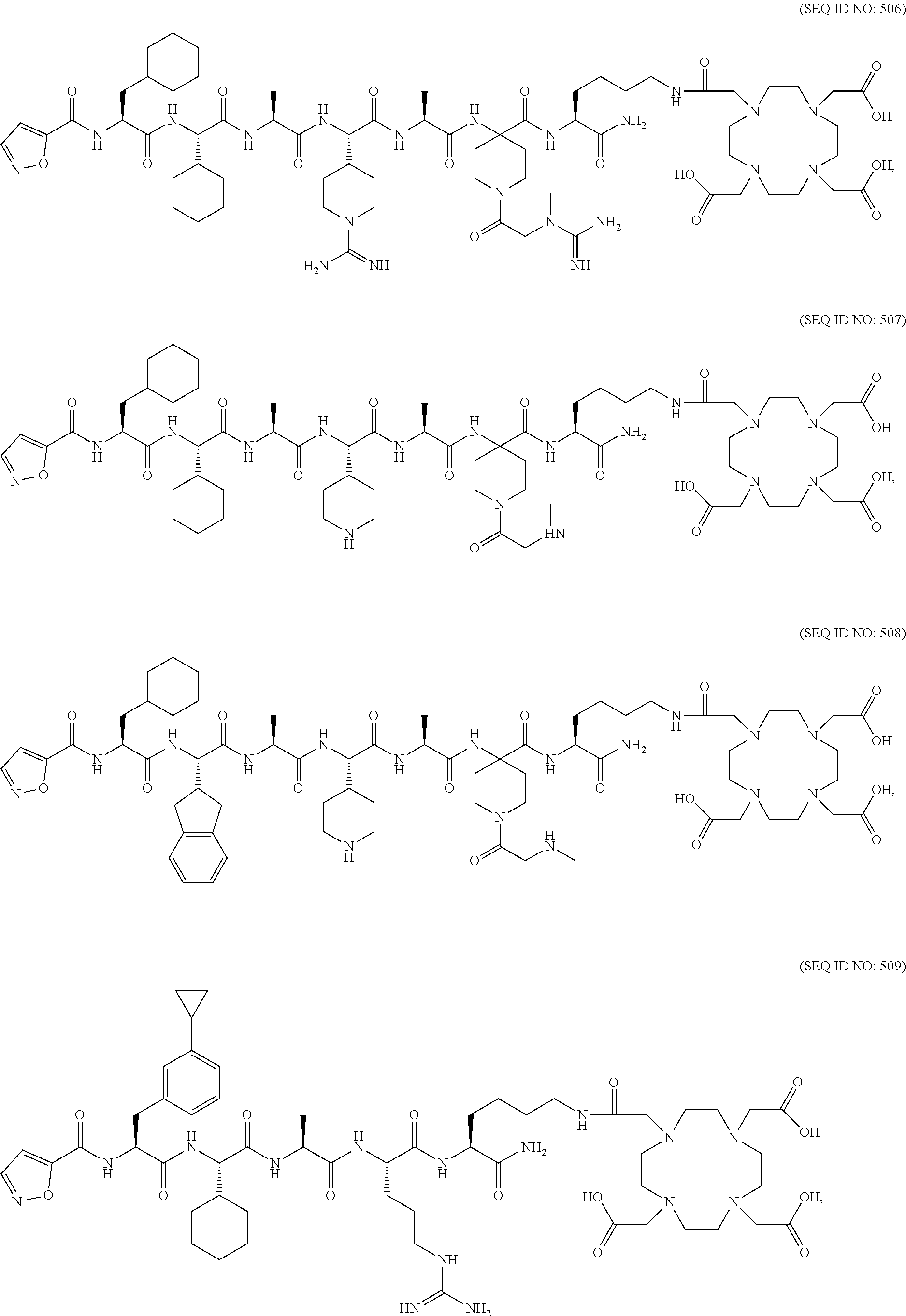

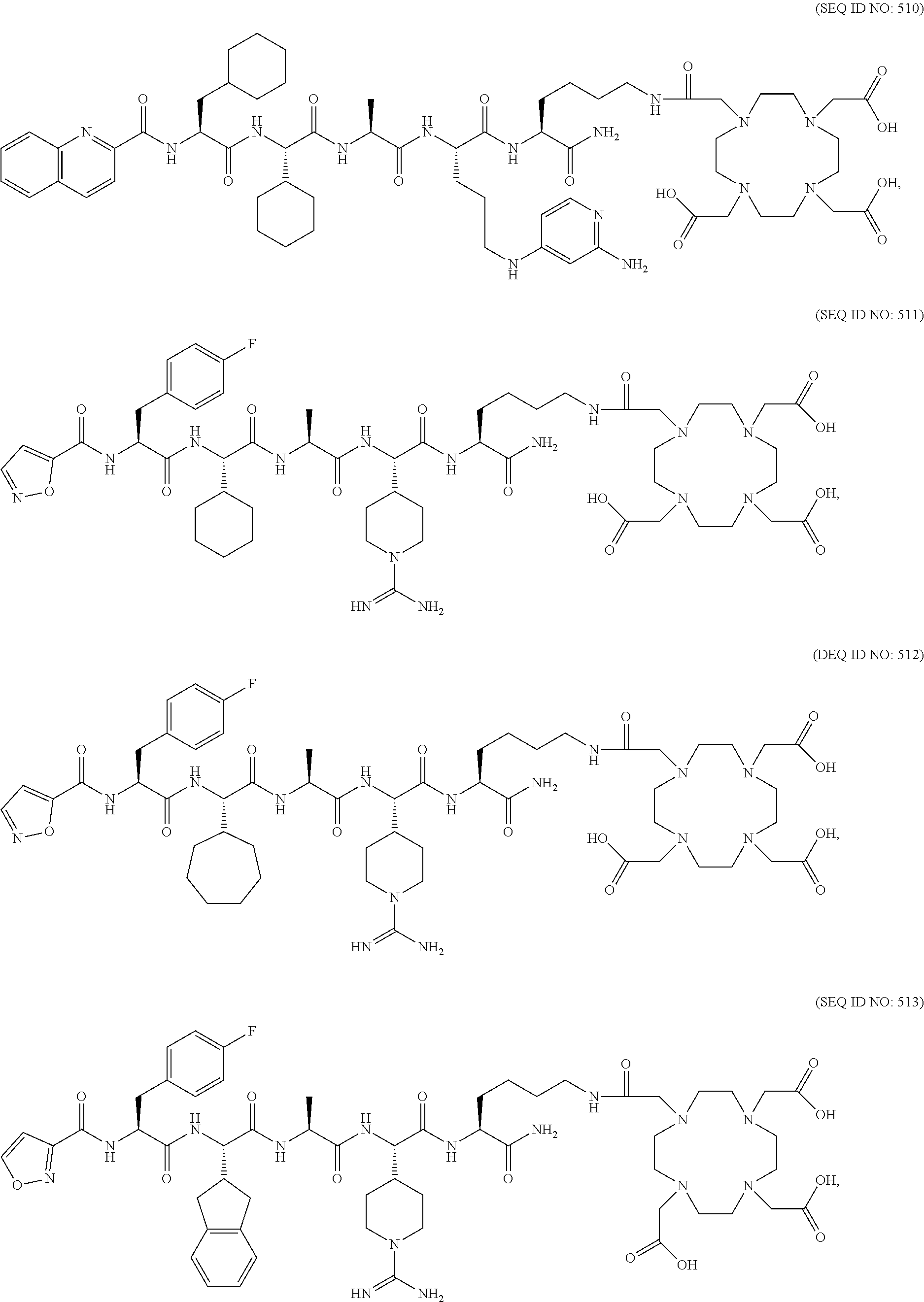

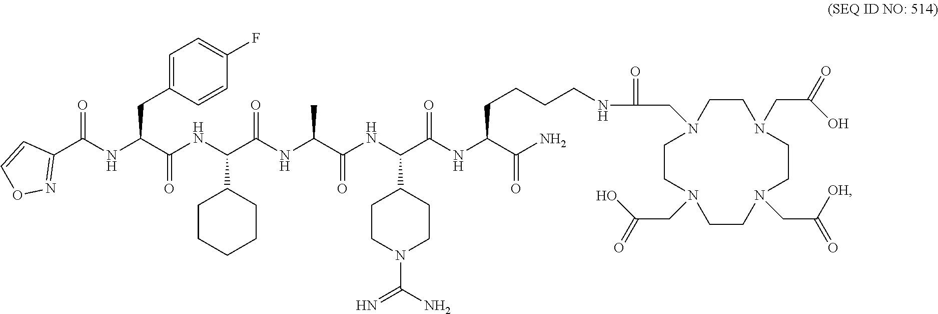 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

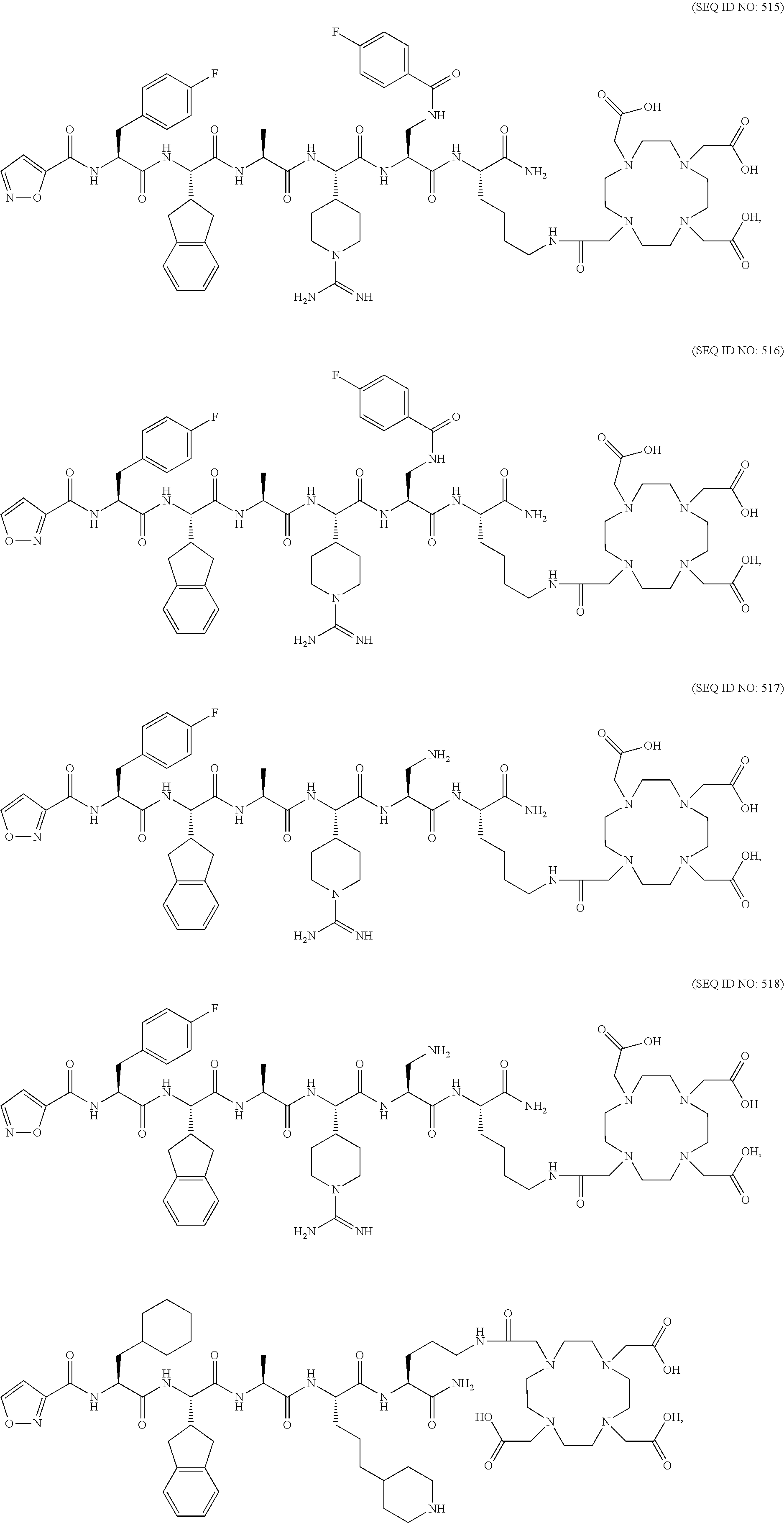

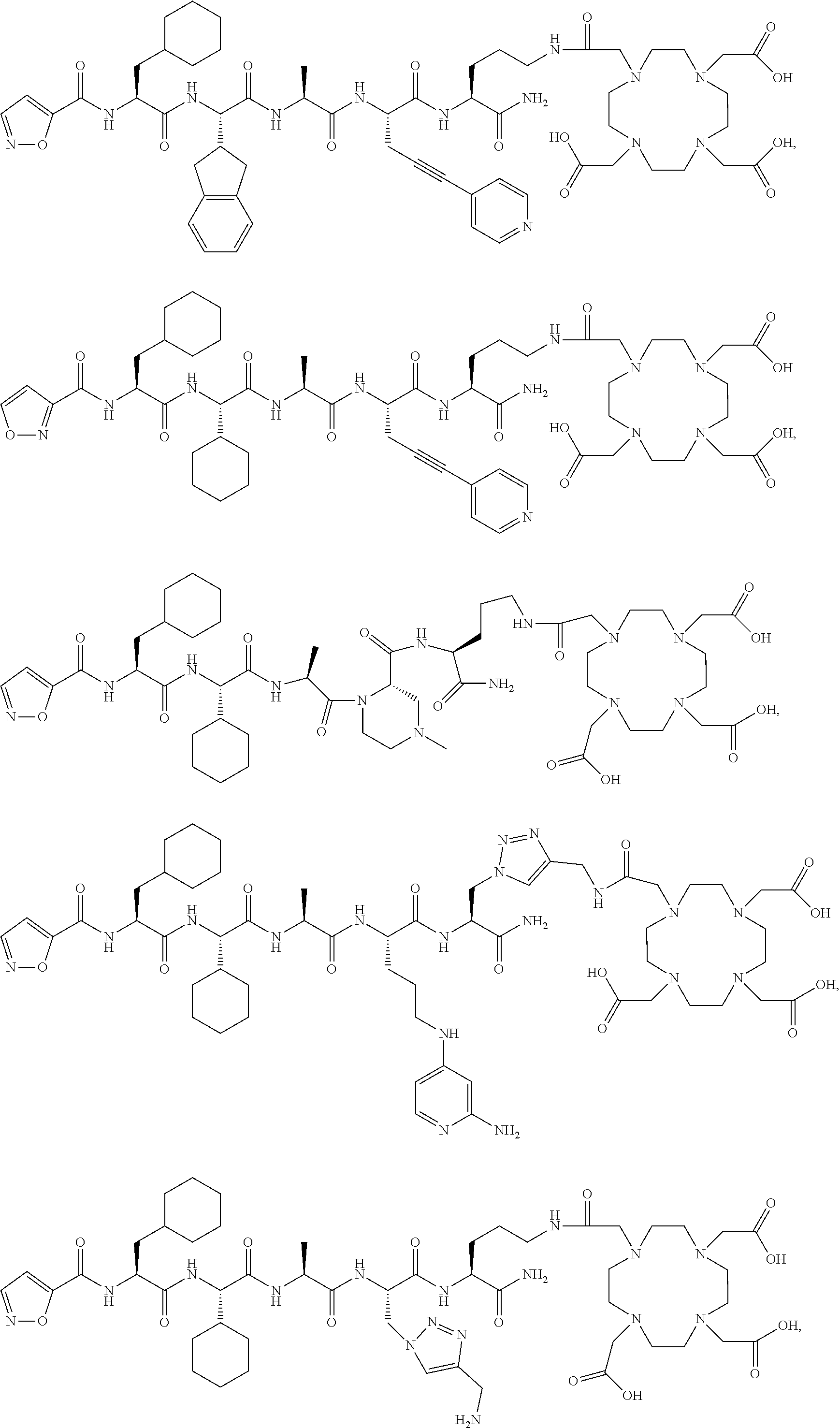

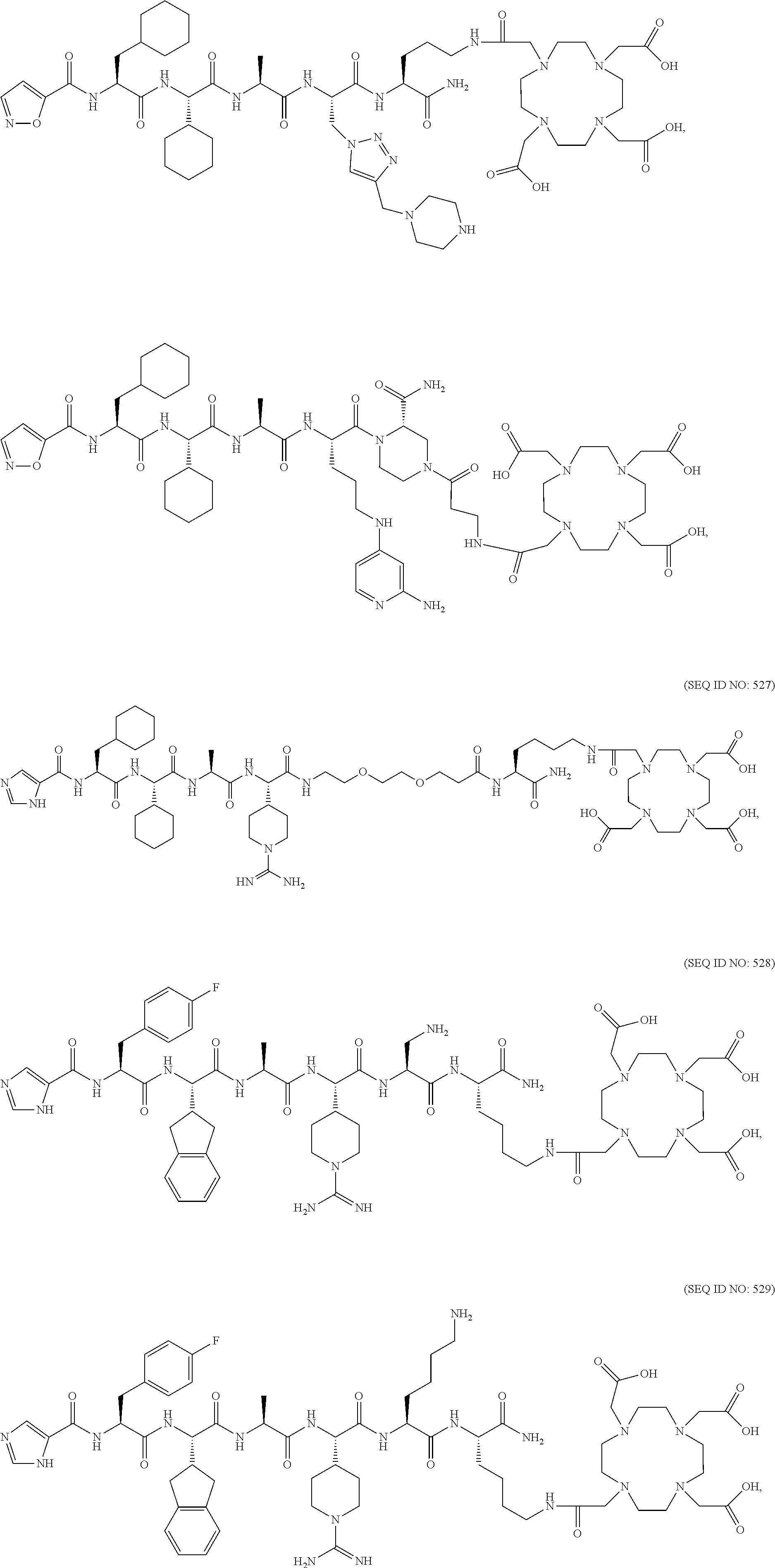

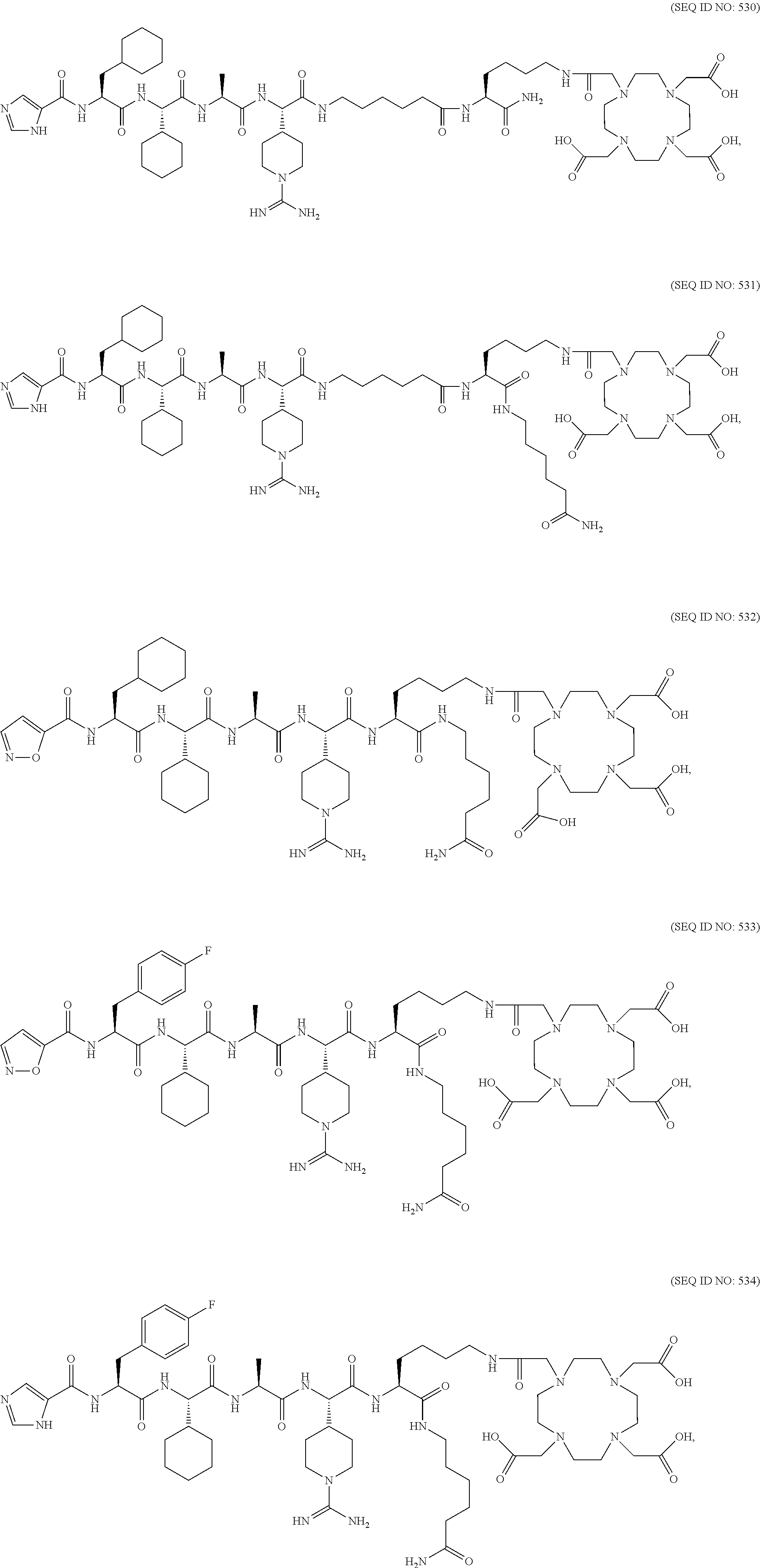

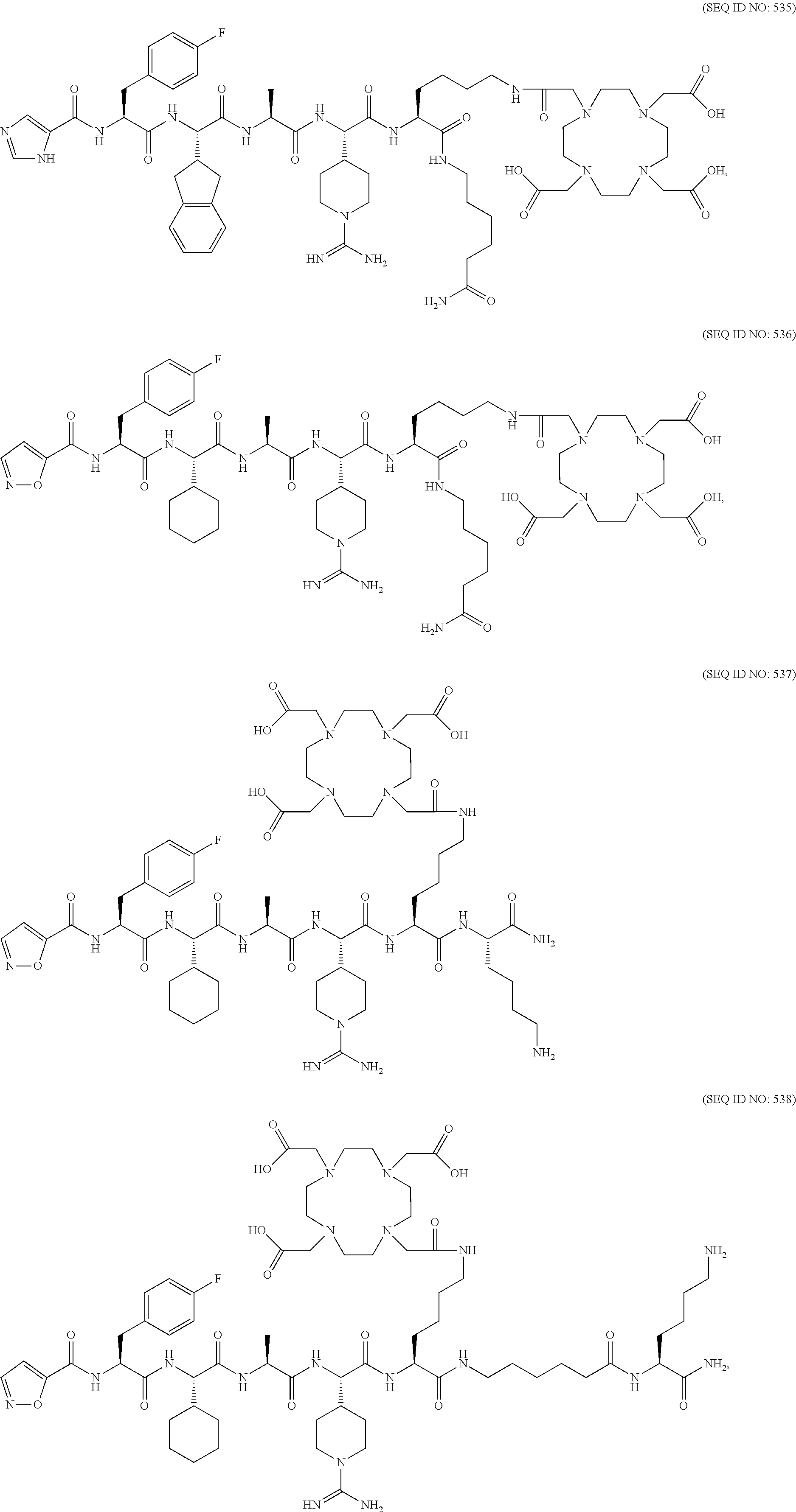

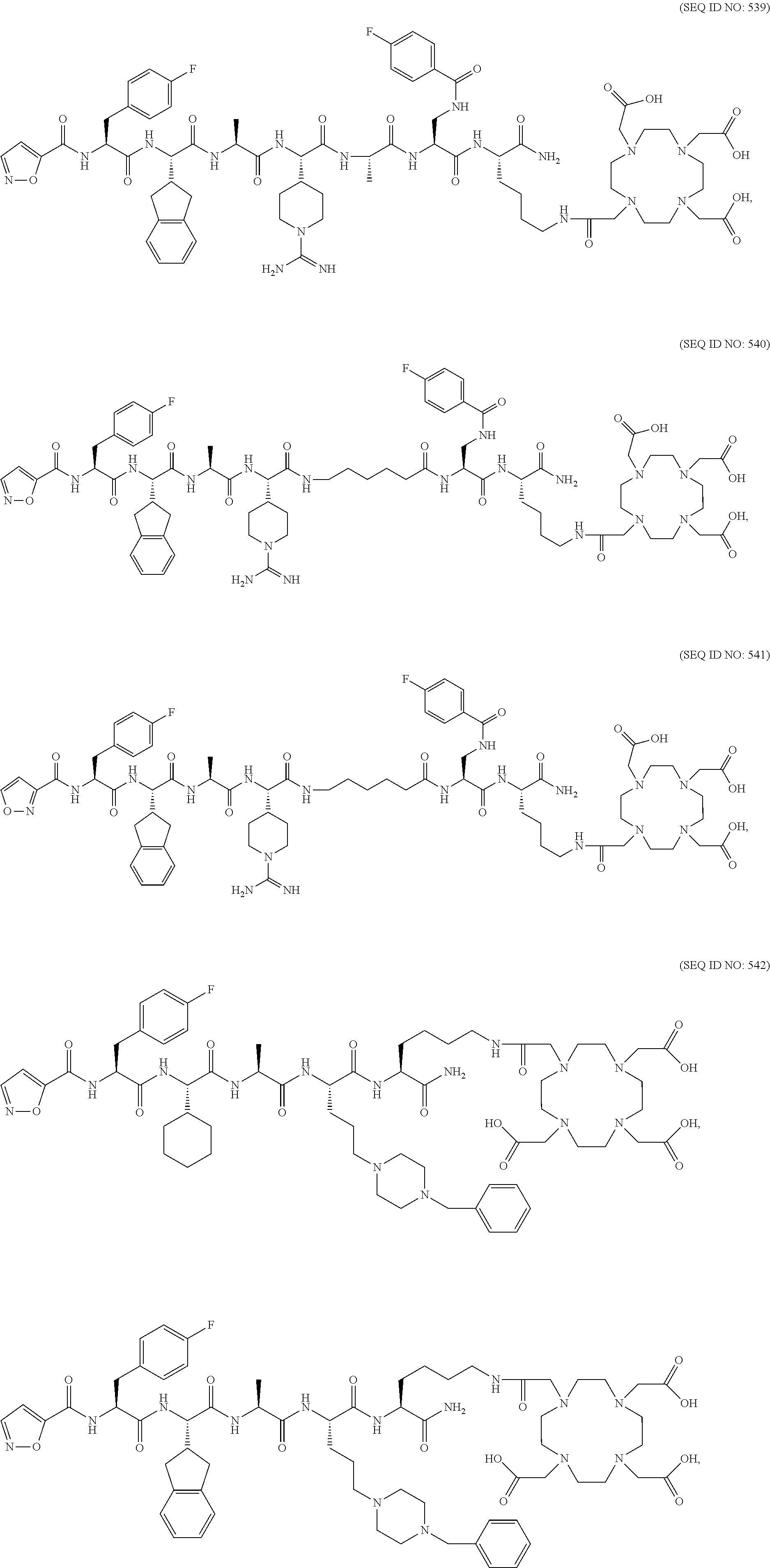

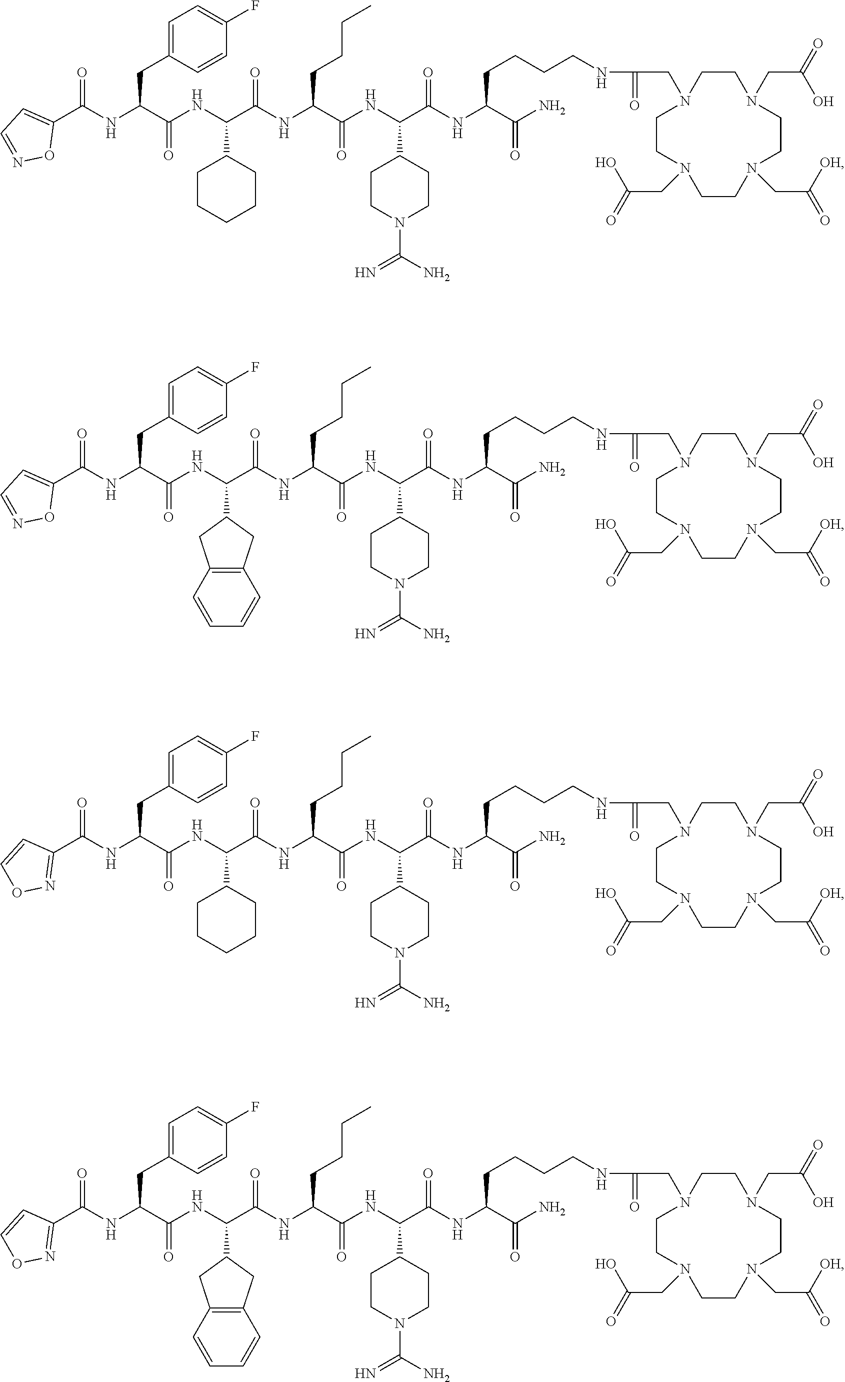

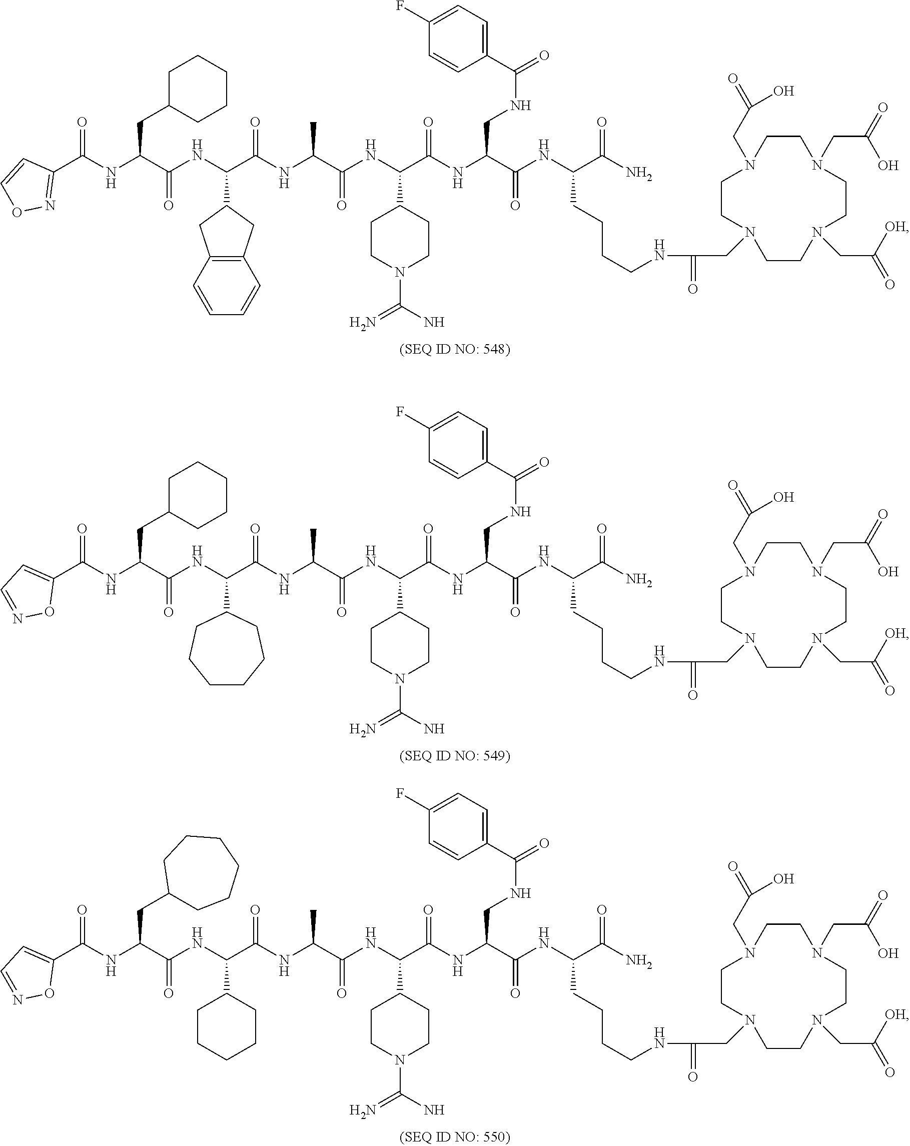

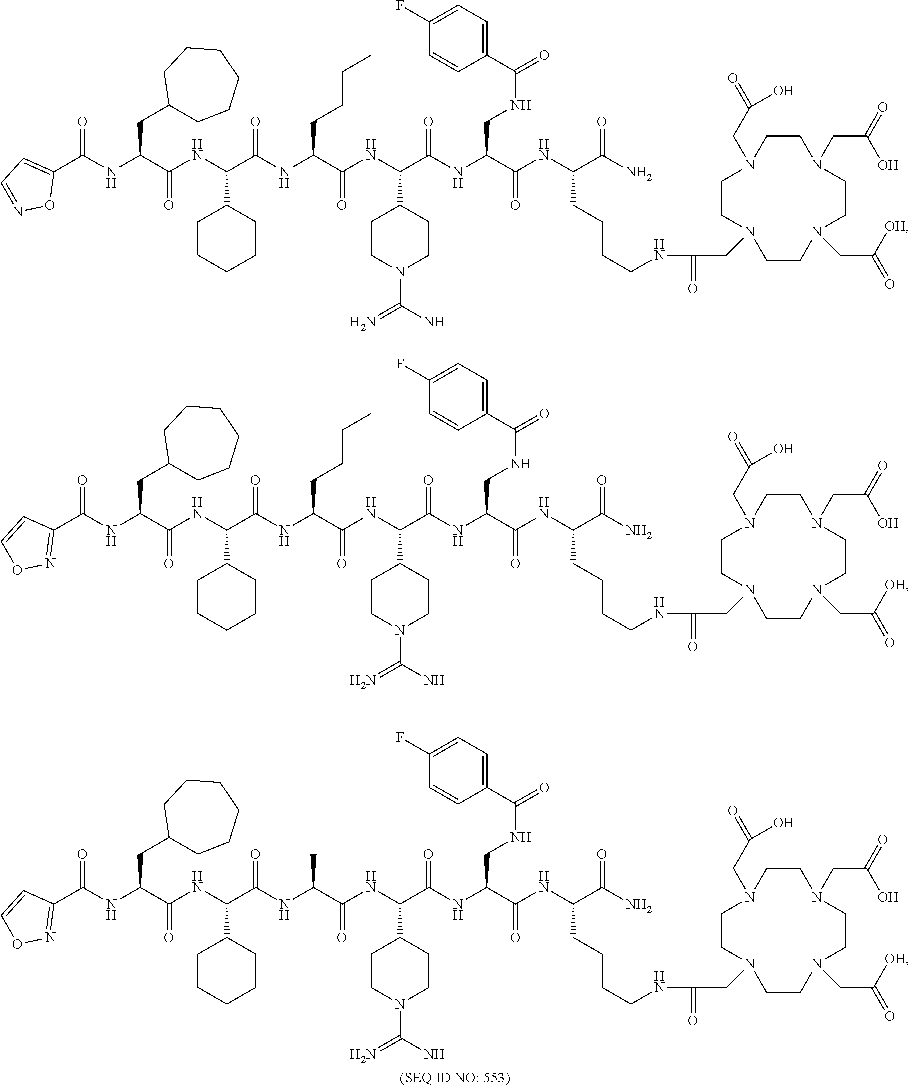

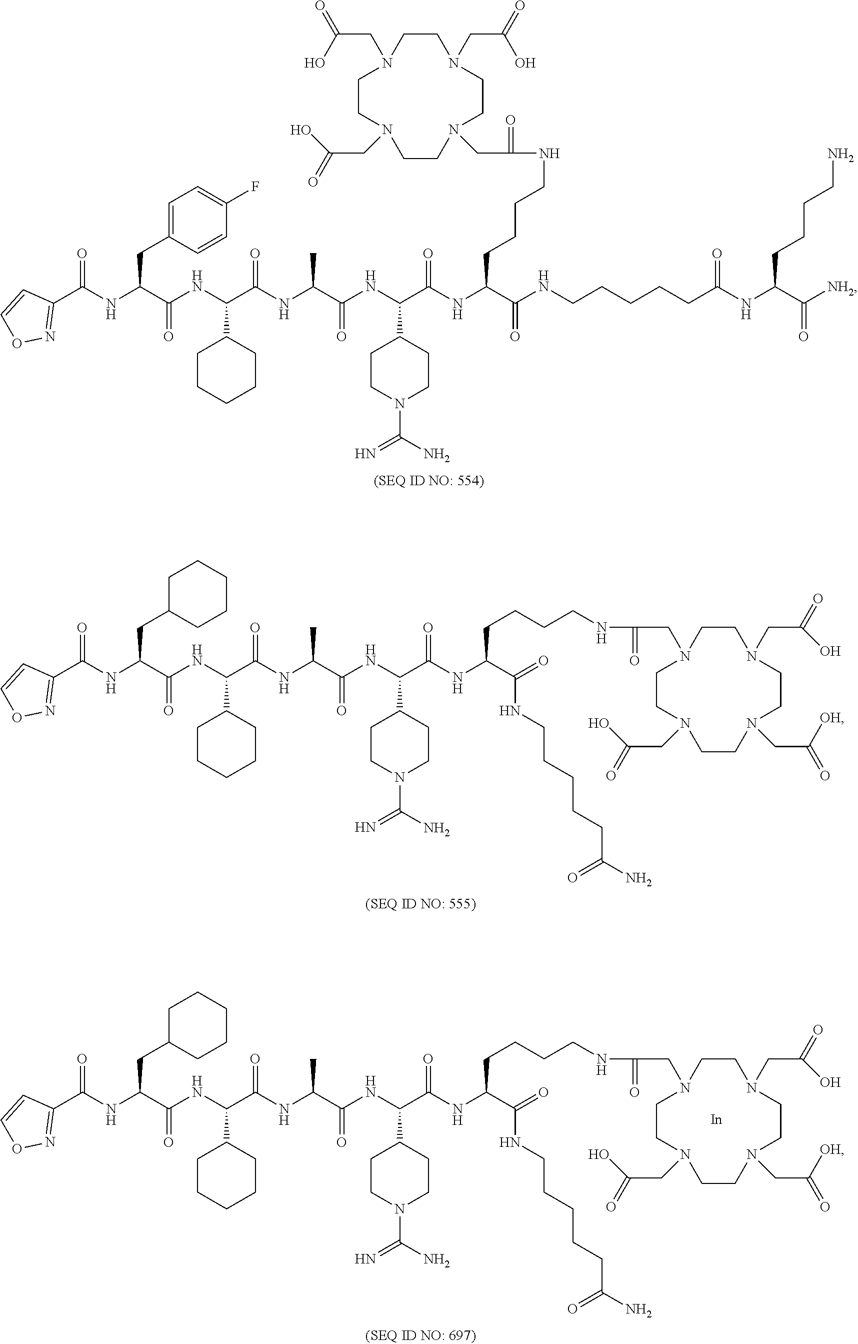

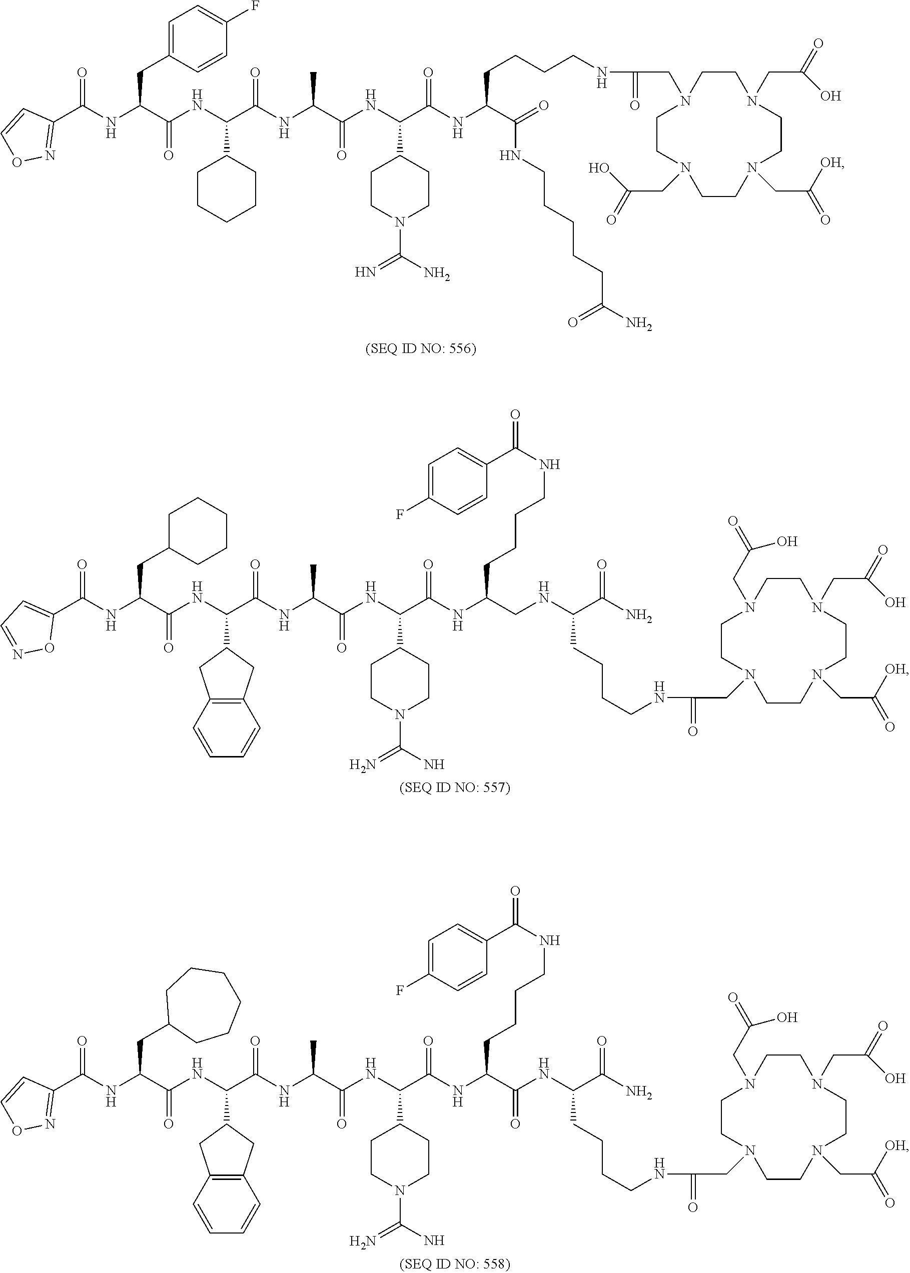

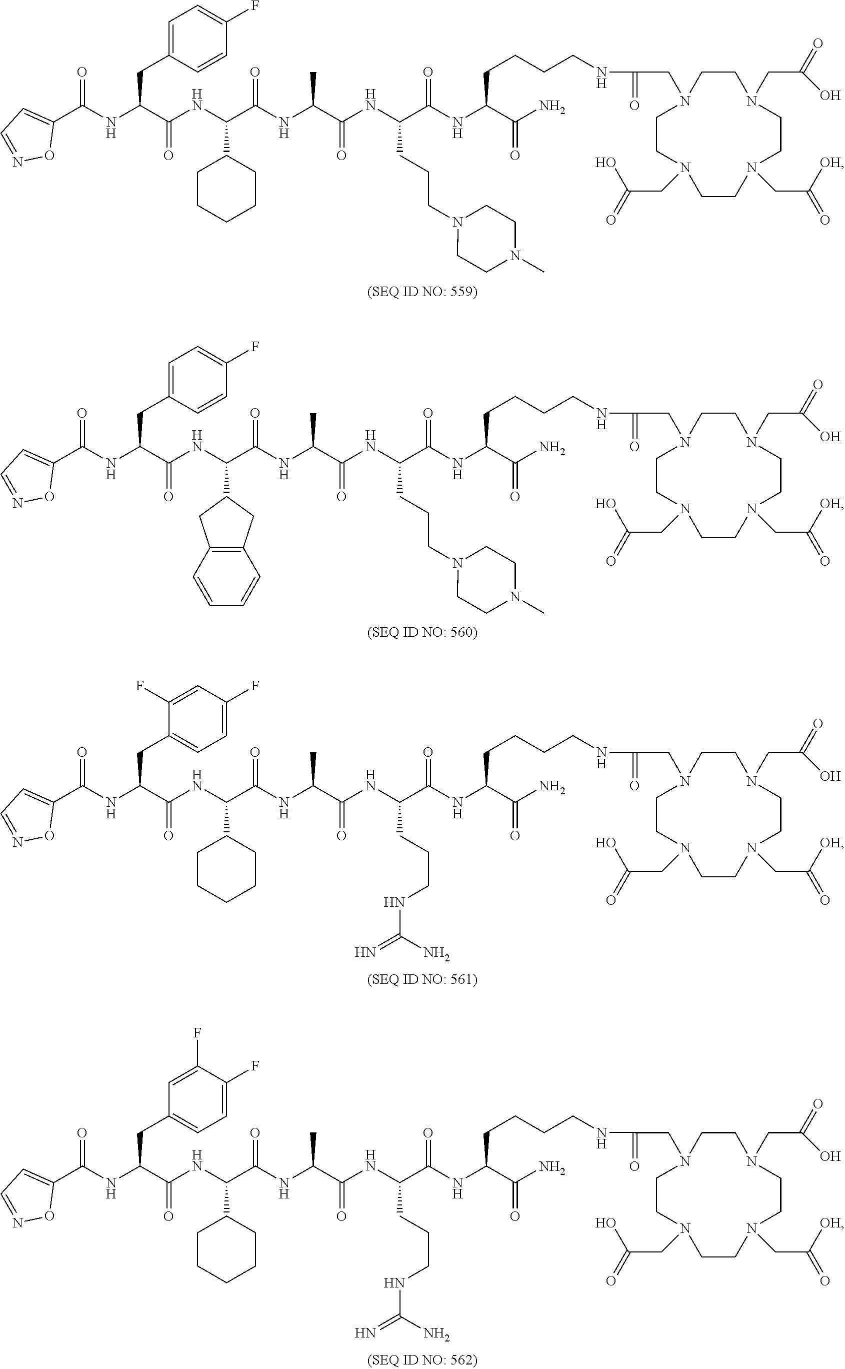

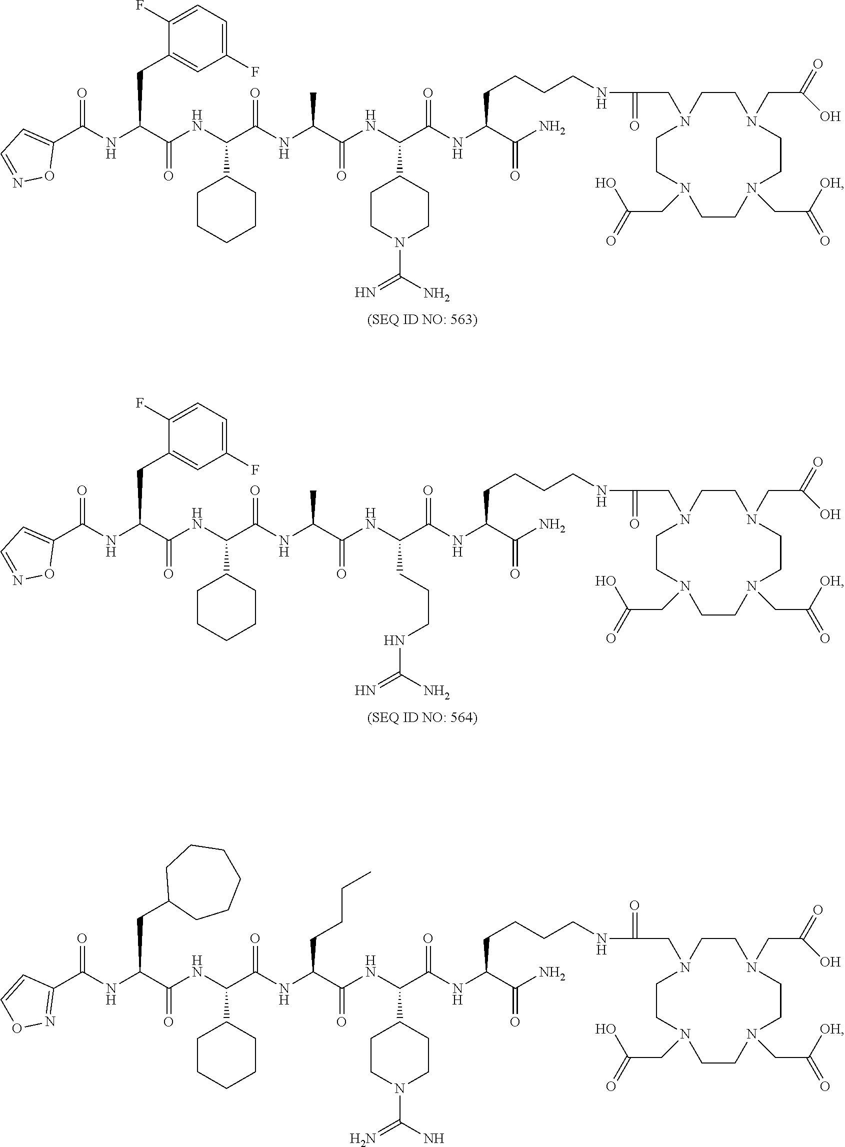

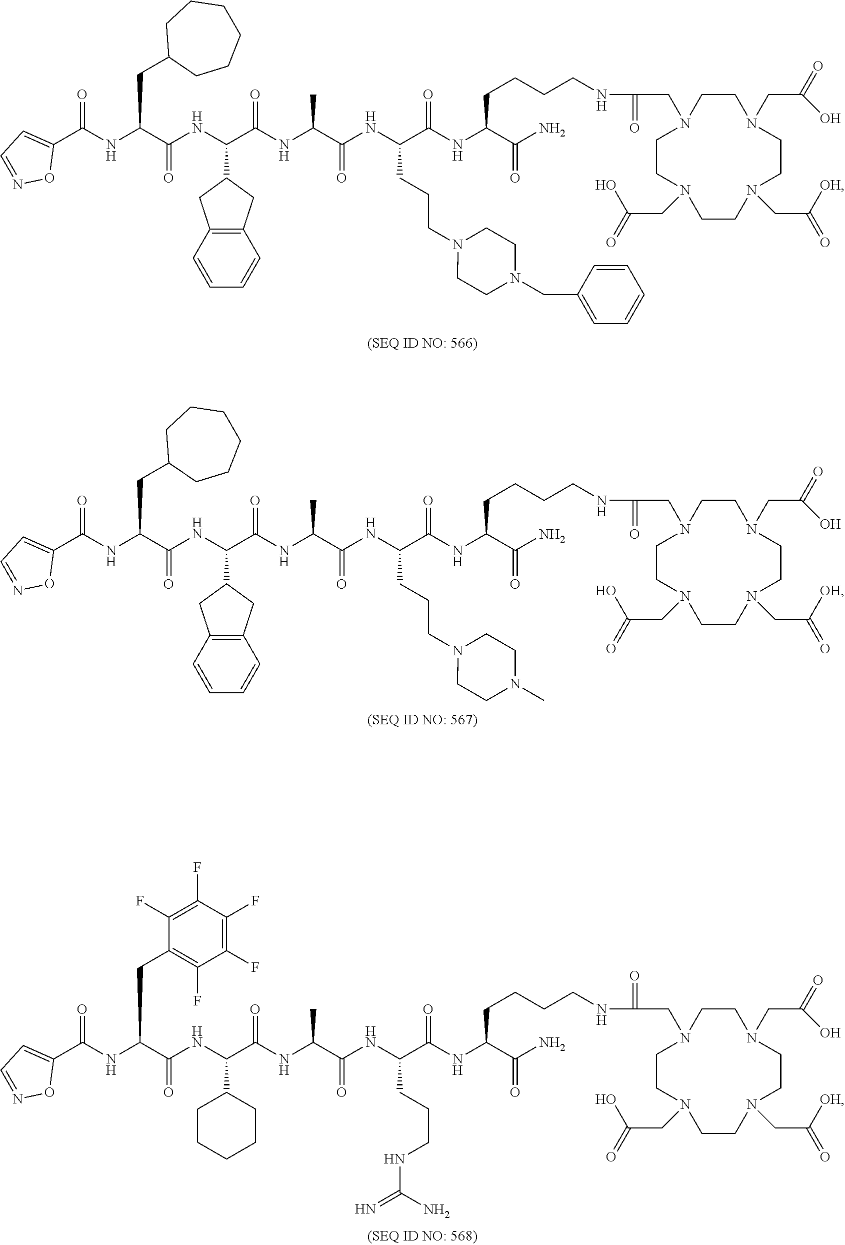

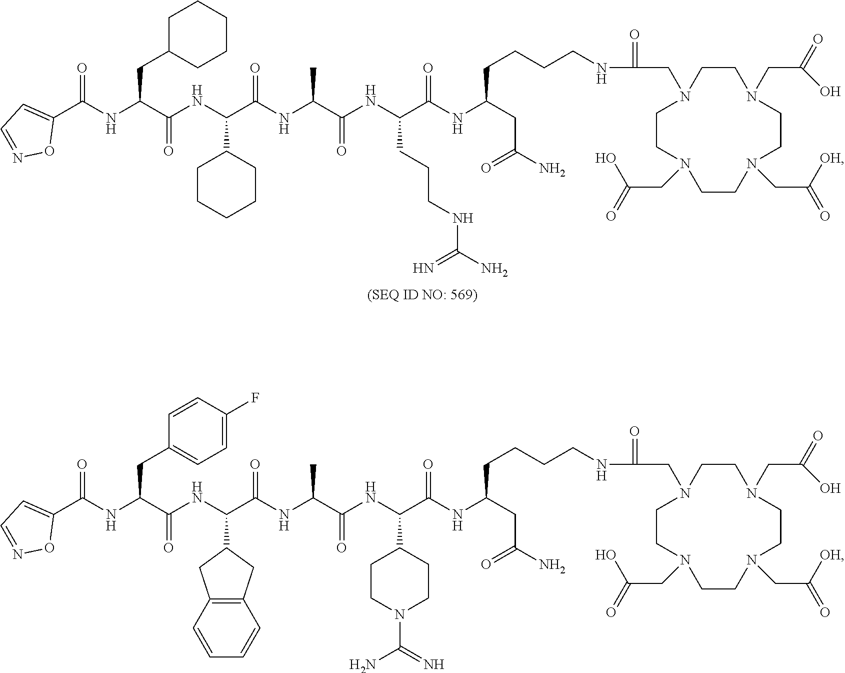 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)CC(N)=O ; C[C@H](NC(=O)[C@@H](NC(=O)[C@H](Cc1ccc(F)cc1)NC(=O)c1ccno1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)CC(N)=O)C1CCN(C(=N)N)CC1]

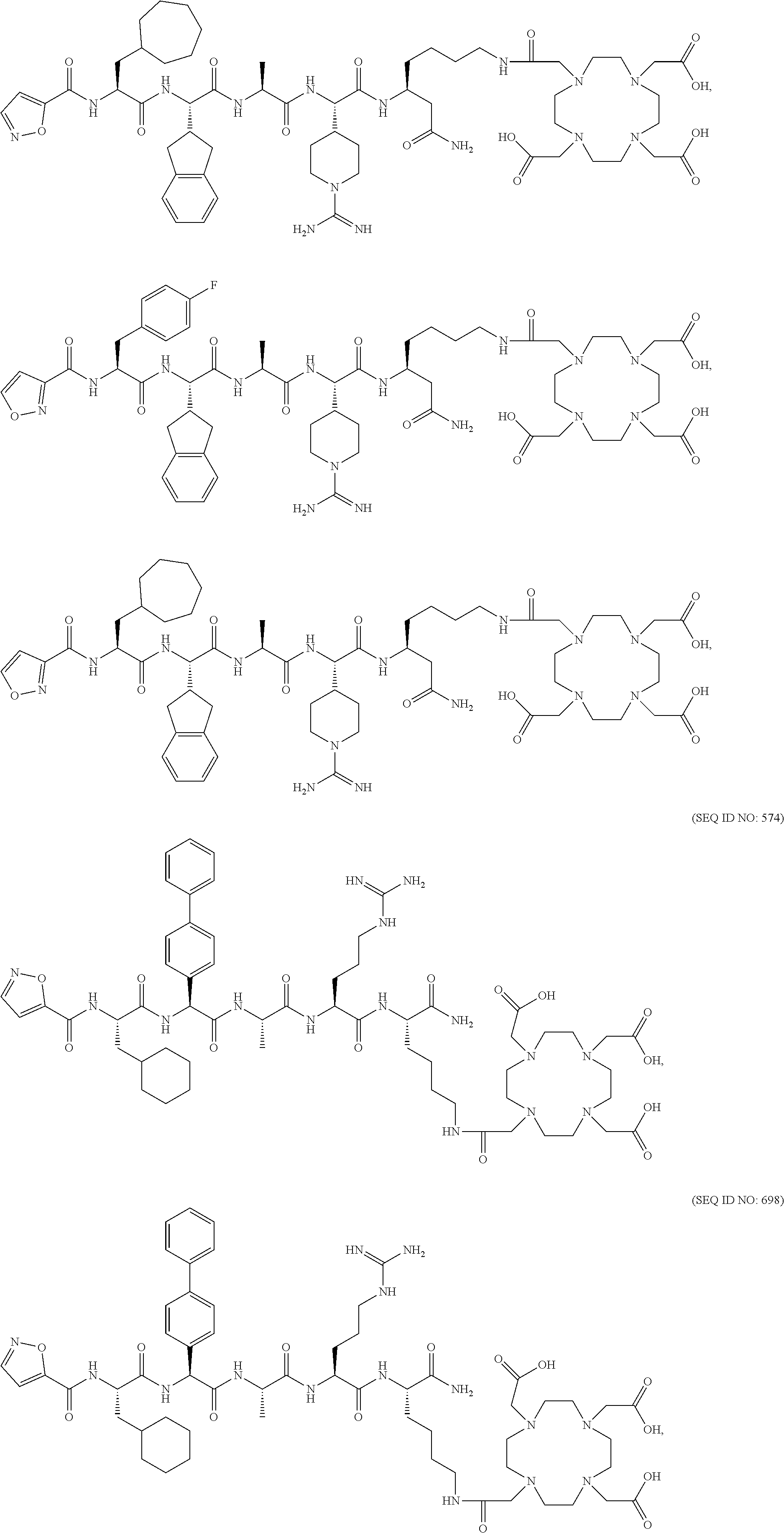

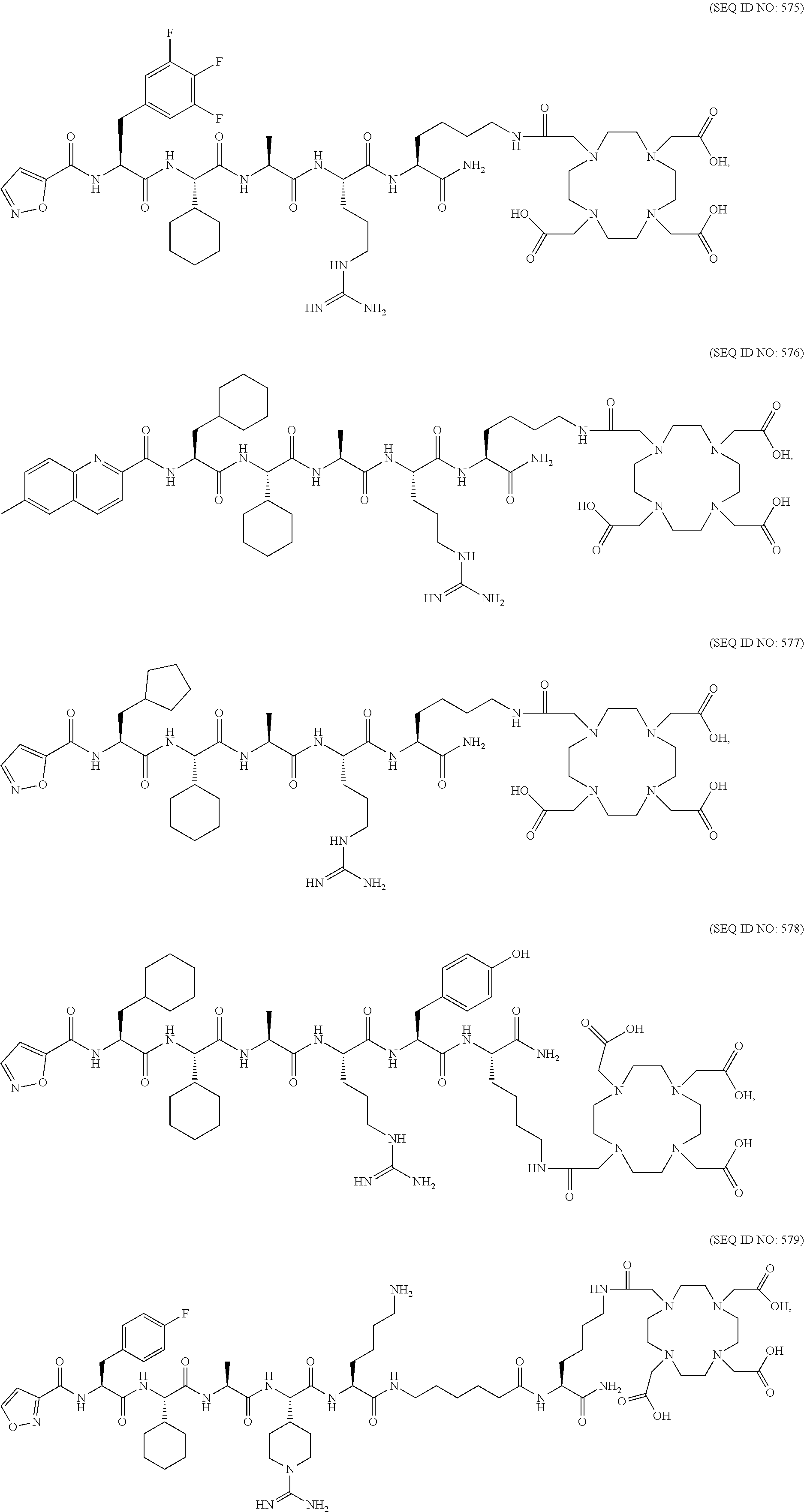

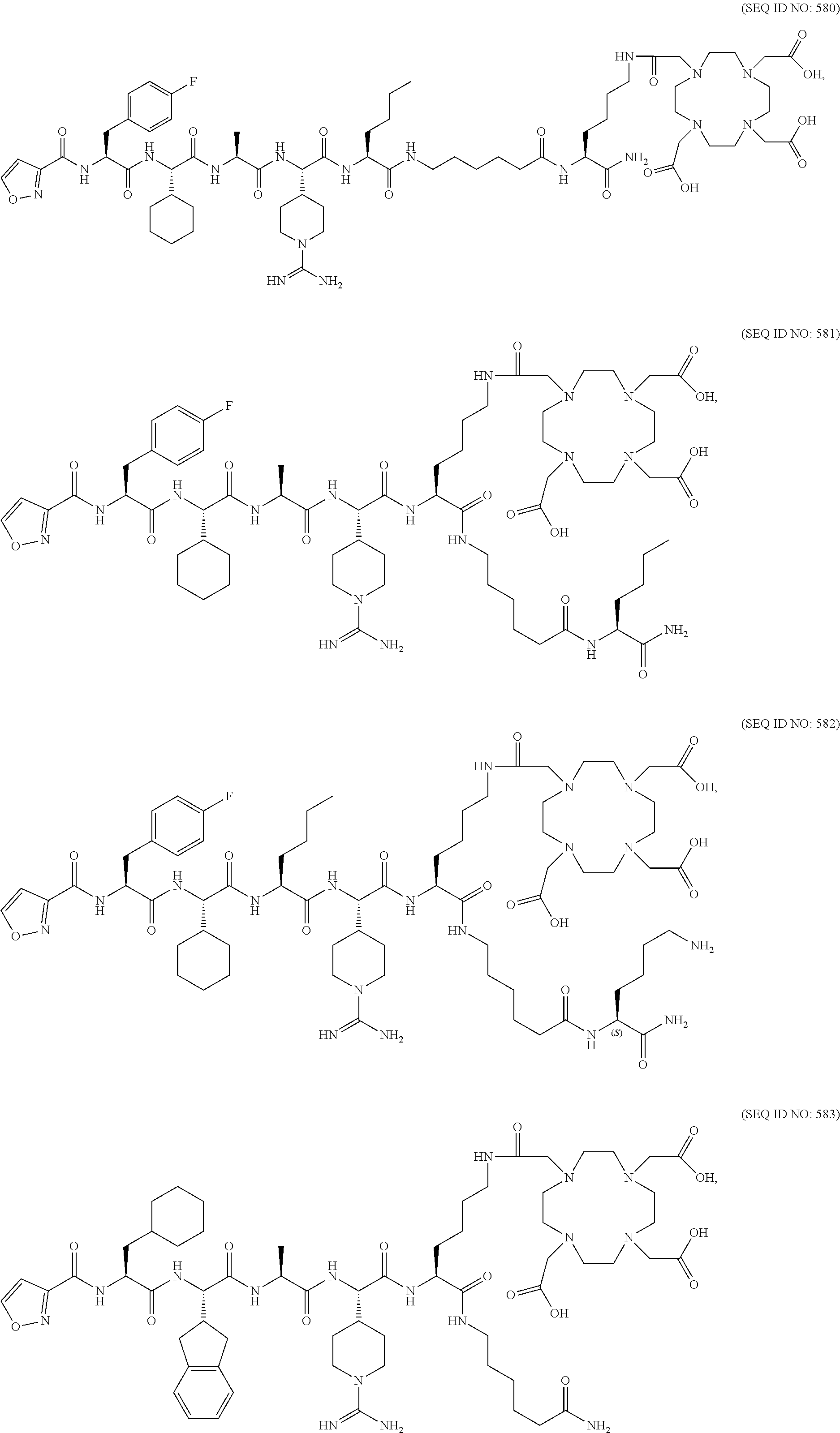

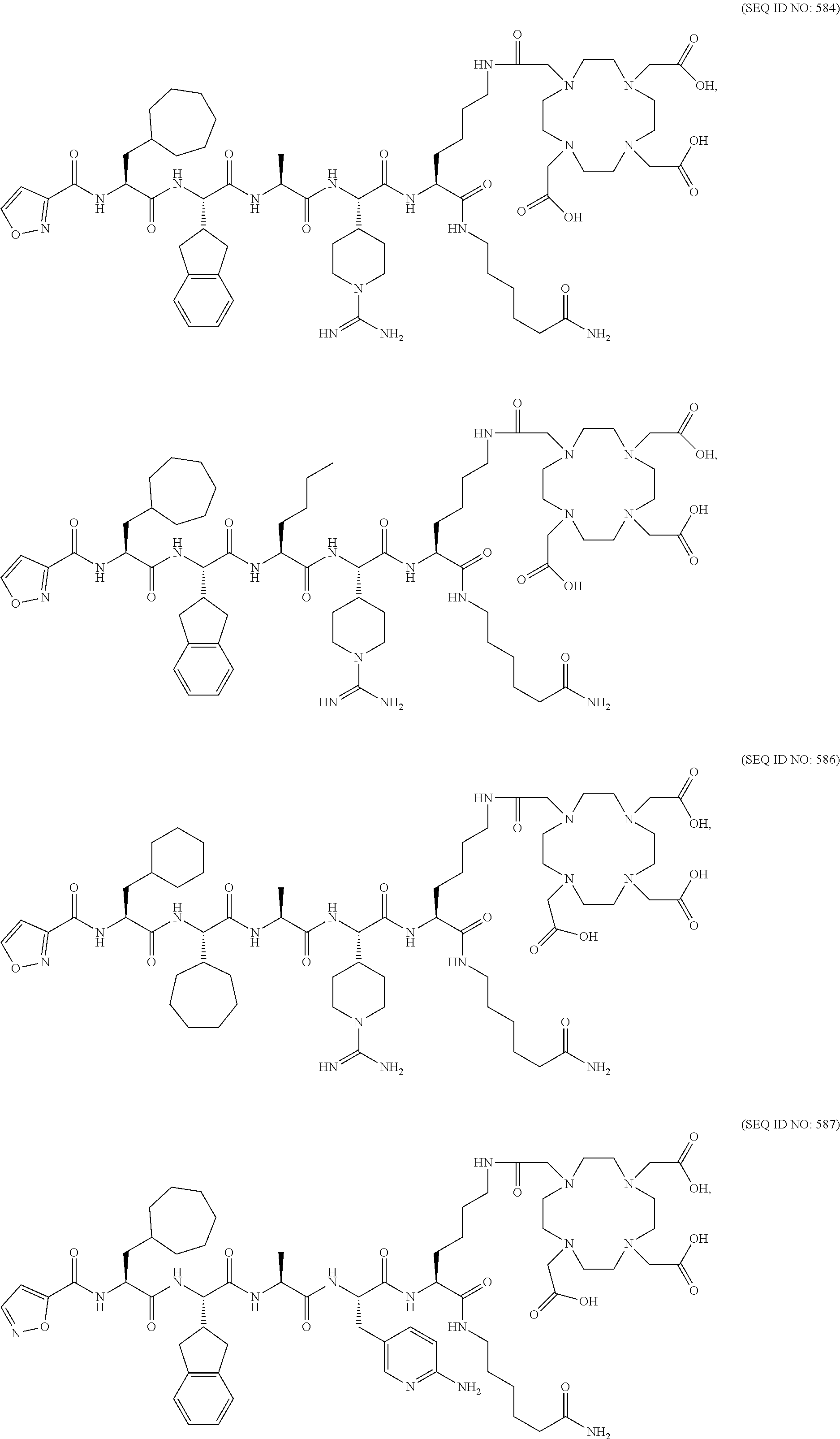

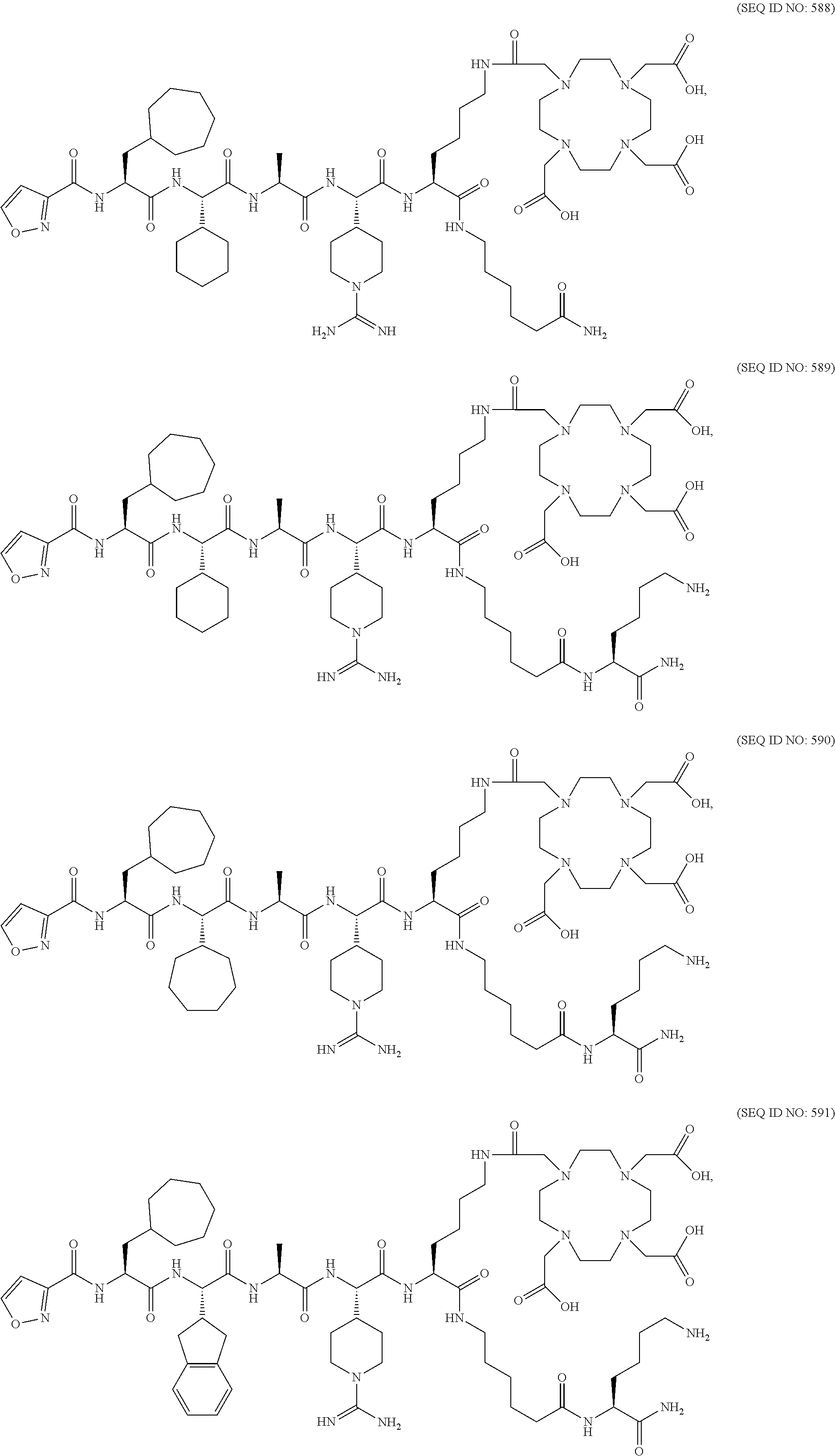

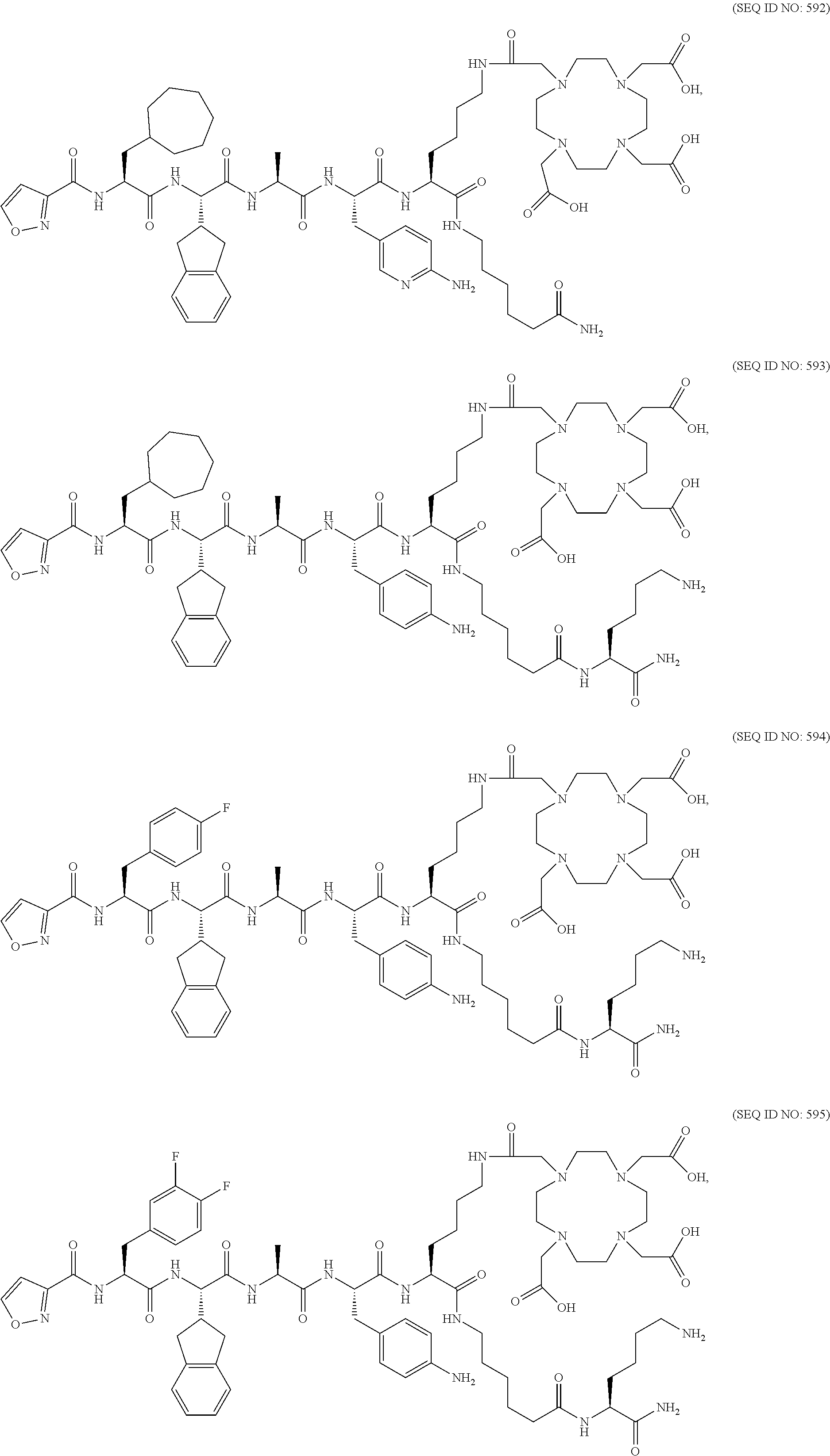

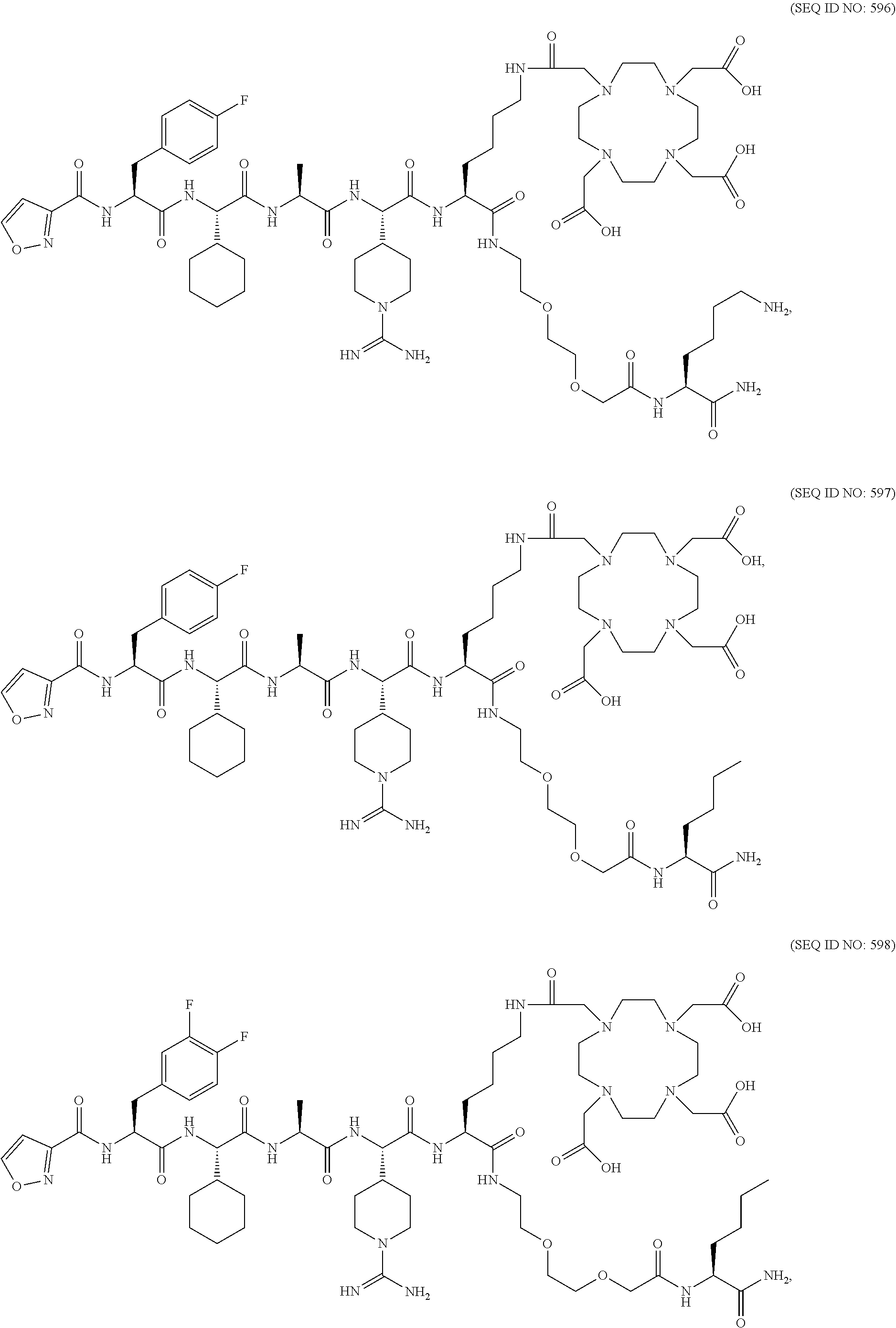

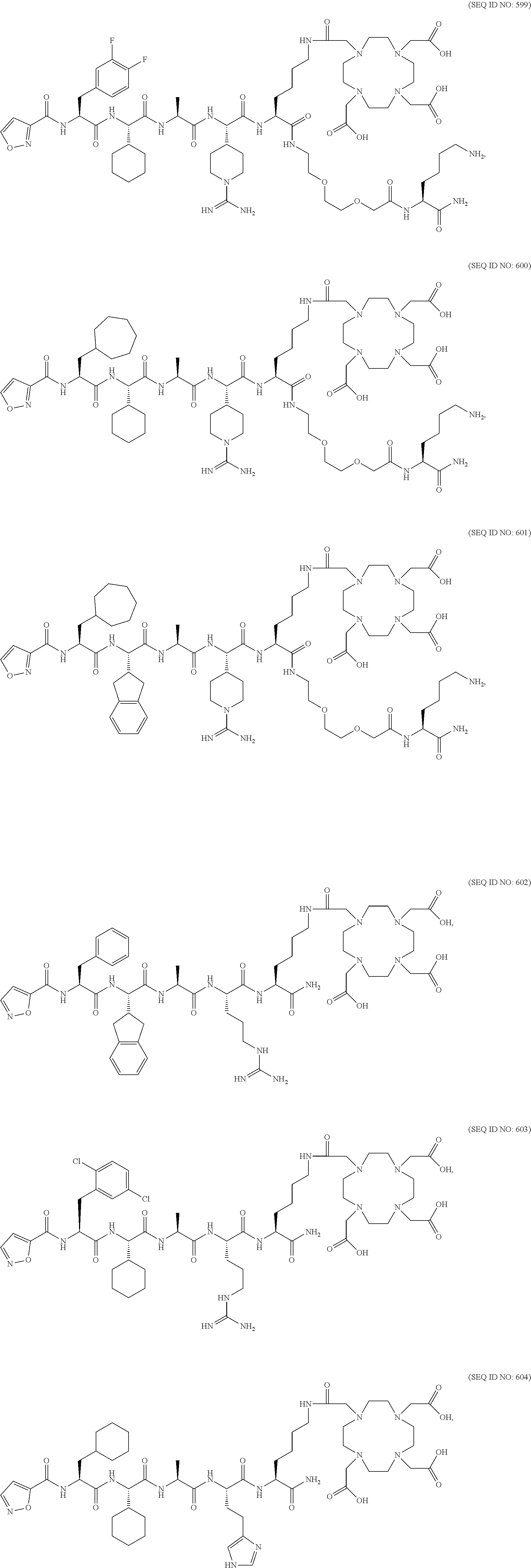

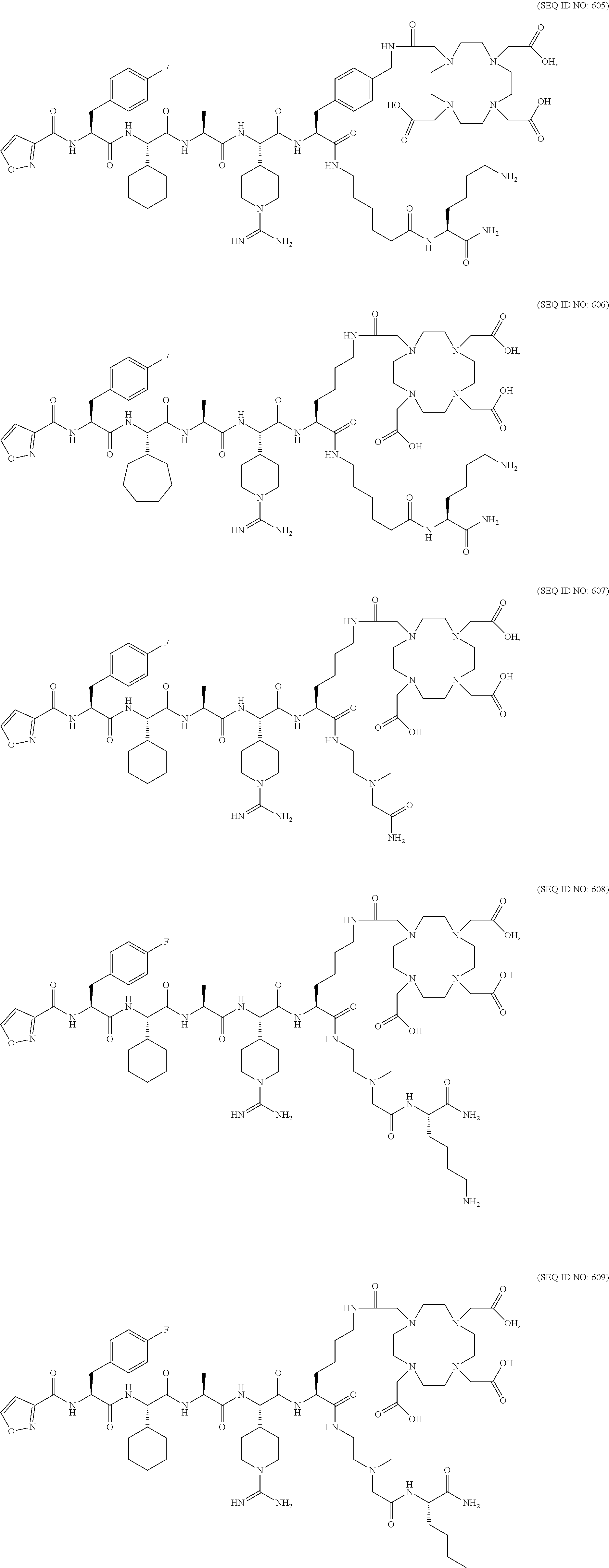

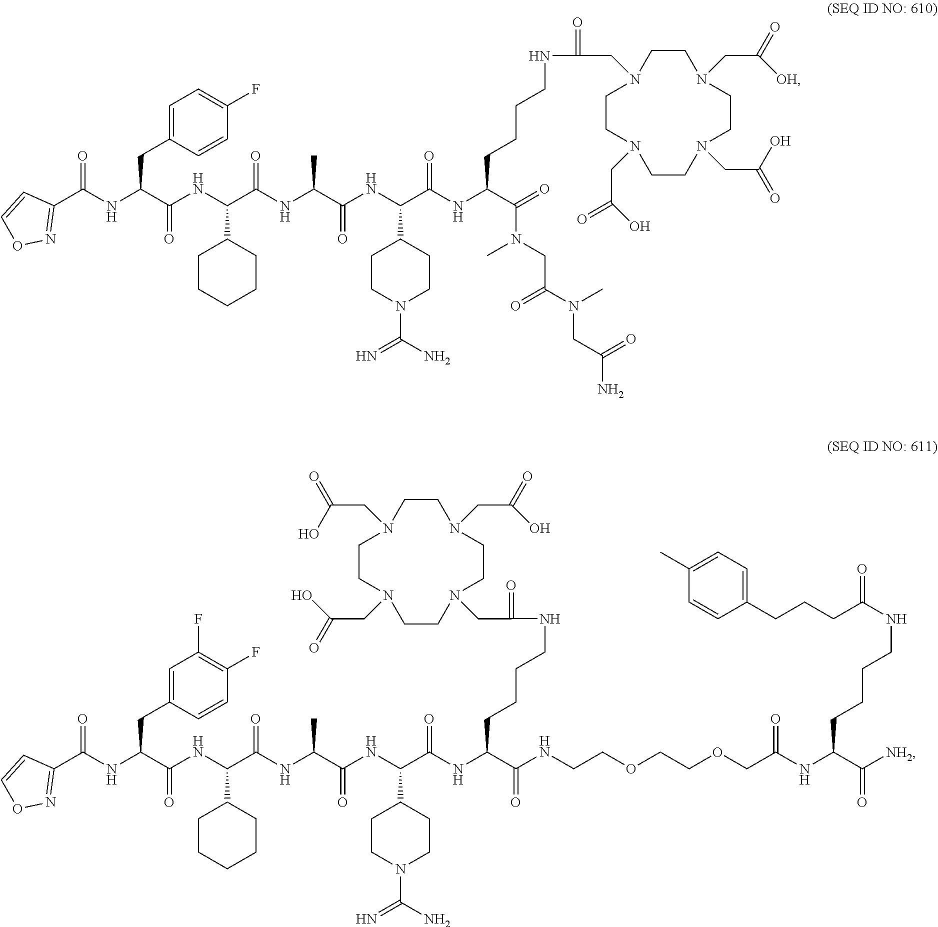 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)N(C)CC(=O)N(C)CC(N)=O)C1CCN(C(=N)N)CC1 ; Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)C2CCCCC2)C2CCN(C(=N)N)CC2)C(N)=O)cc1]

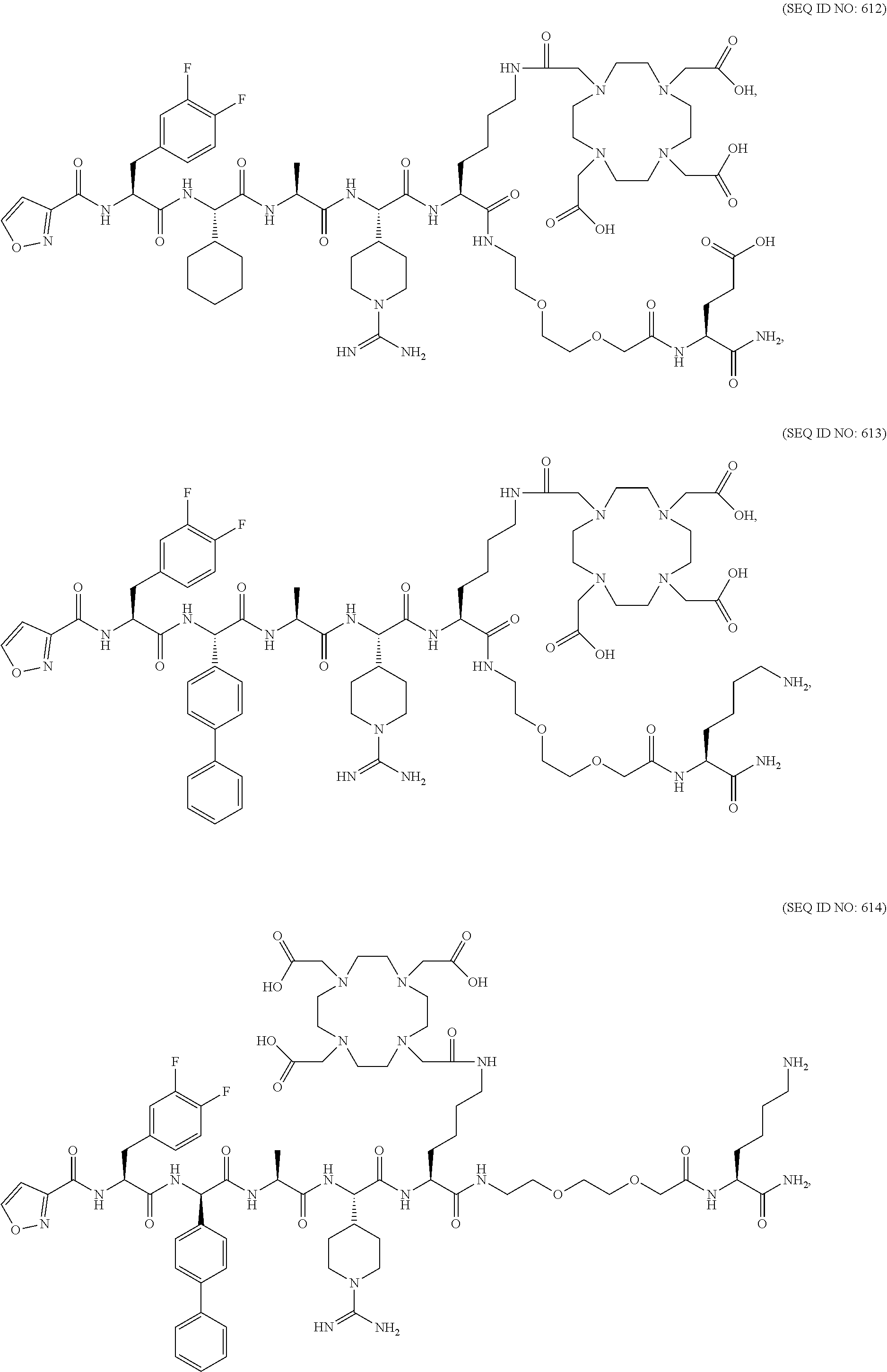

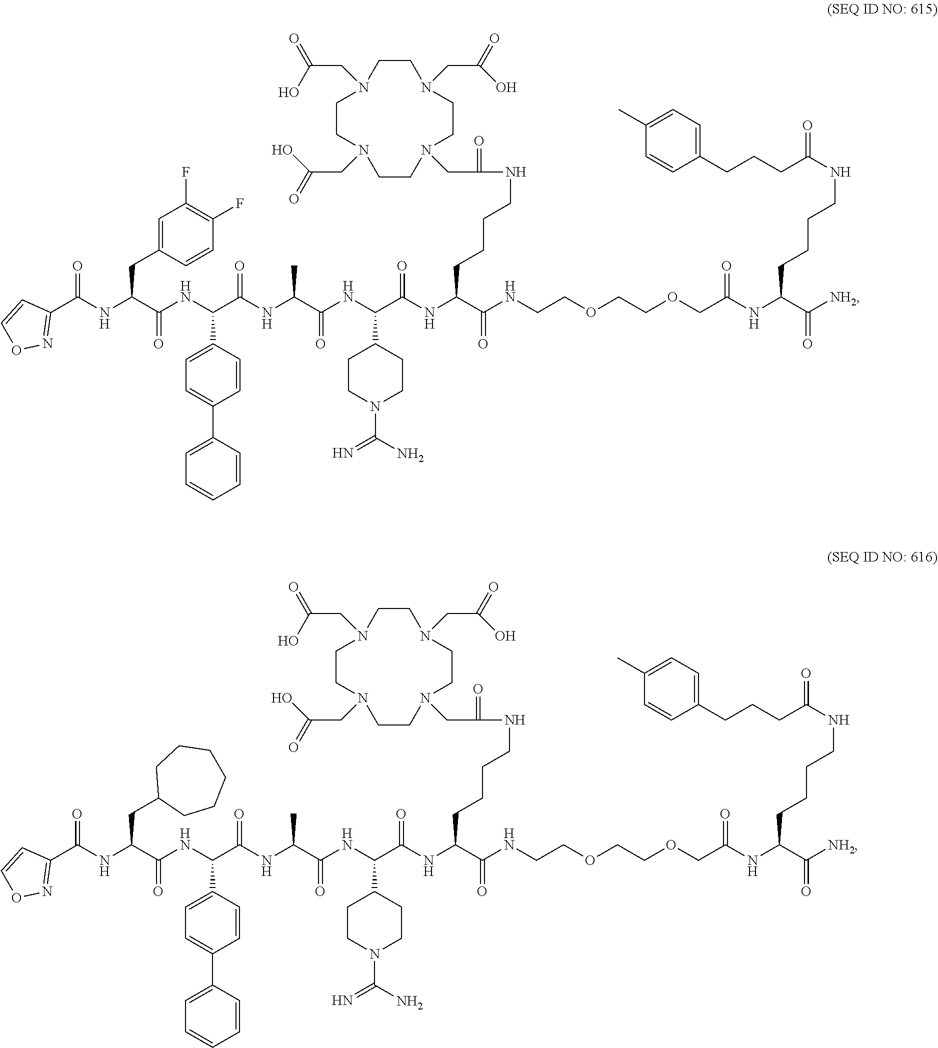 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)c2ccc(-c3ccccc3)cc2)C2CCN(C(=N)N)CC2)C(N)=O)cc1 ; Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](CC2CCCCCC2)NC(=O)c2ccon2)c2ccc(-c3ccccc3)cc2)C2CCN(C(=N)N)CC2)C(N)=O)cc1]

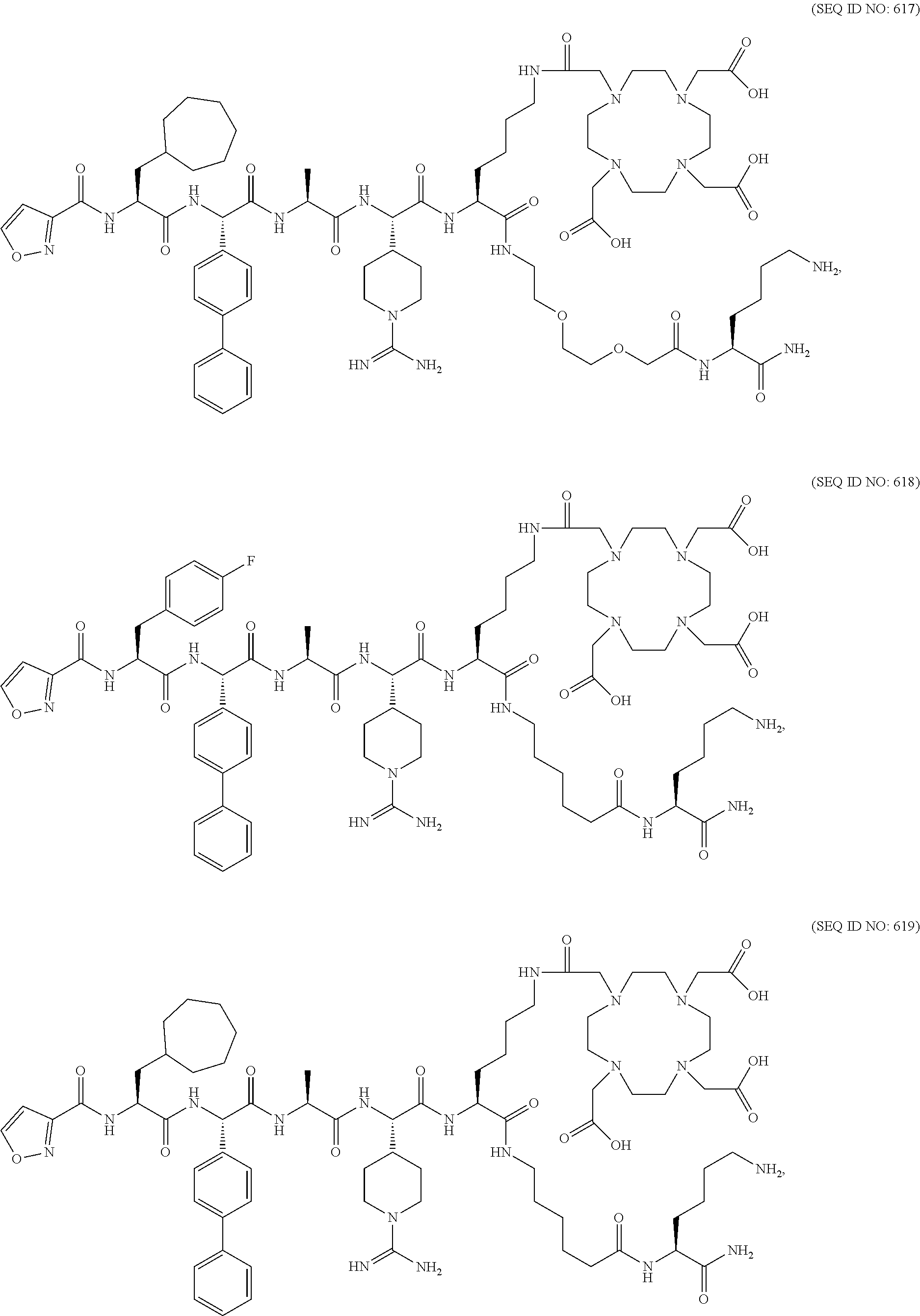

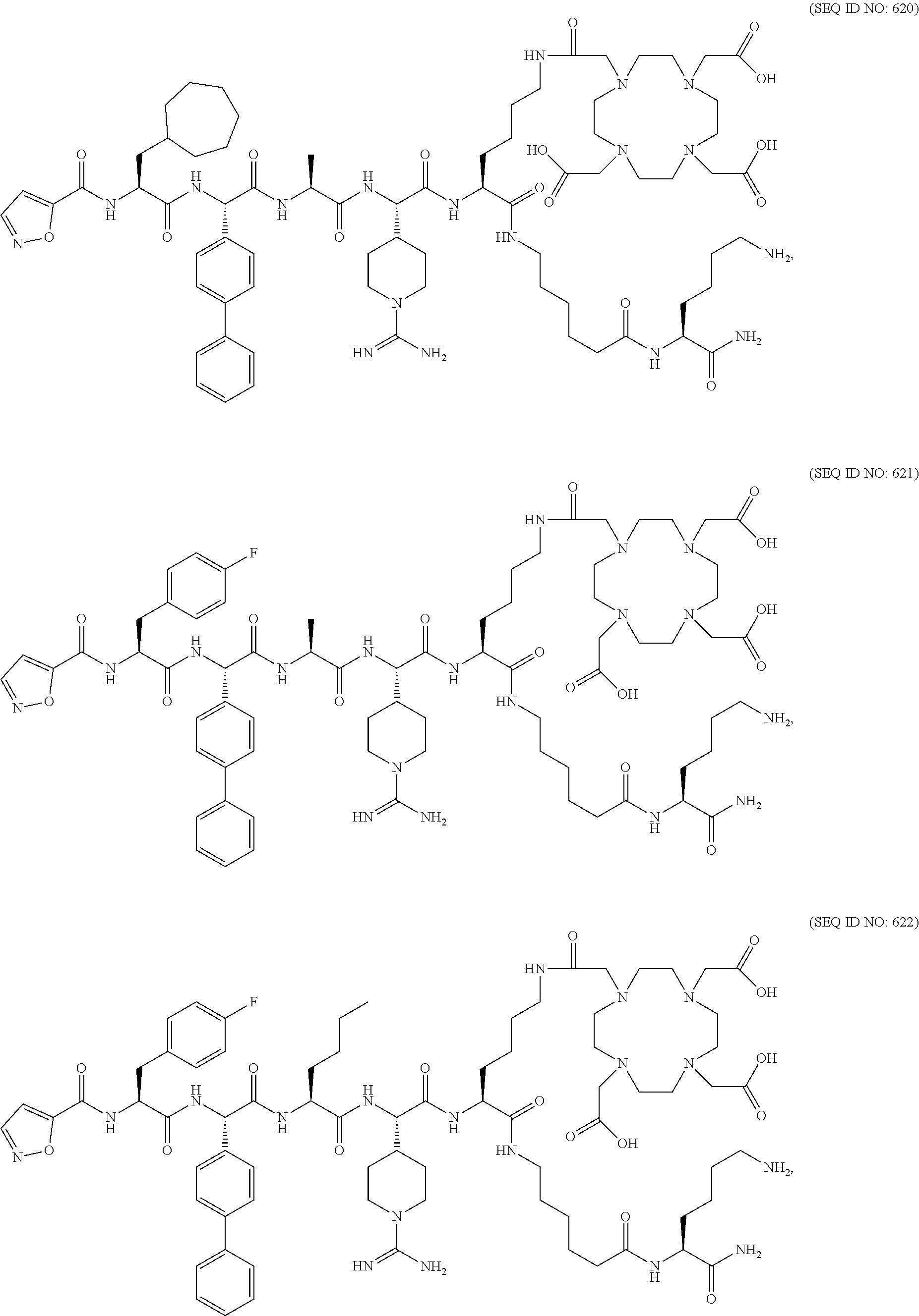

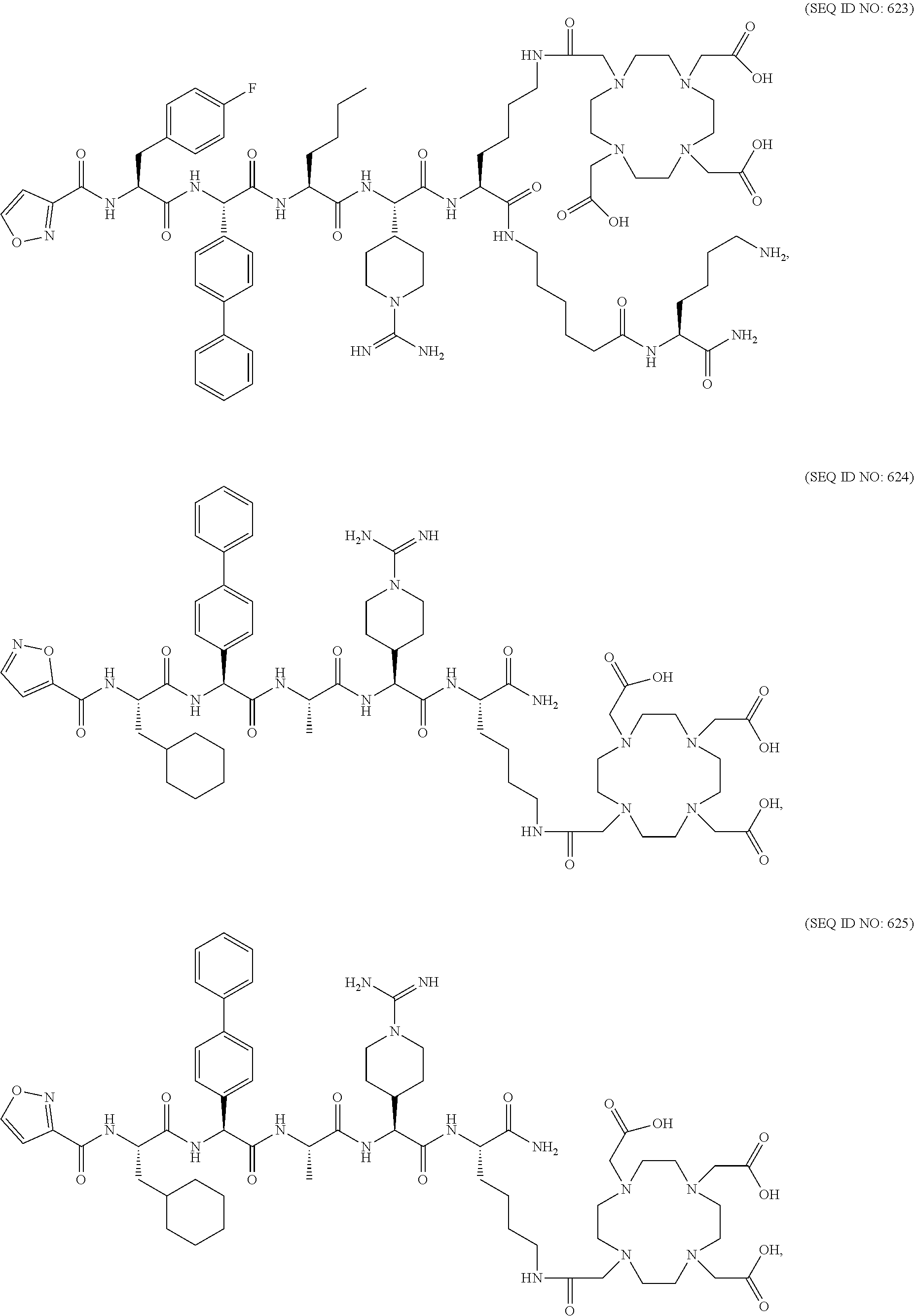

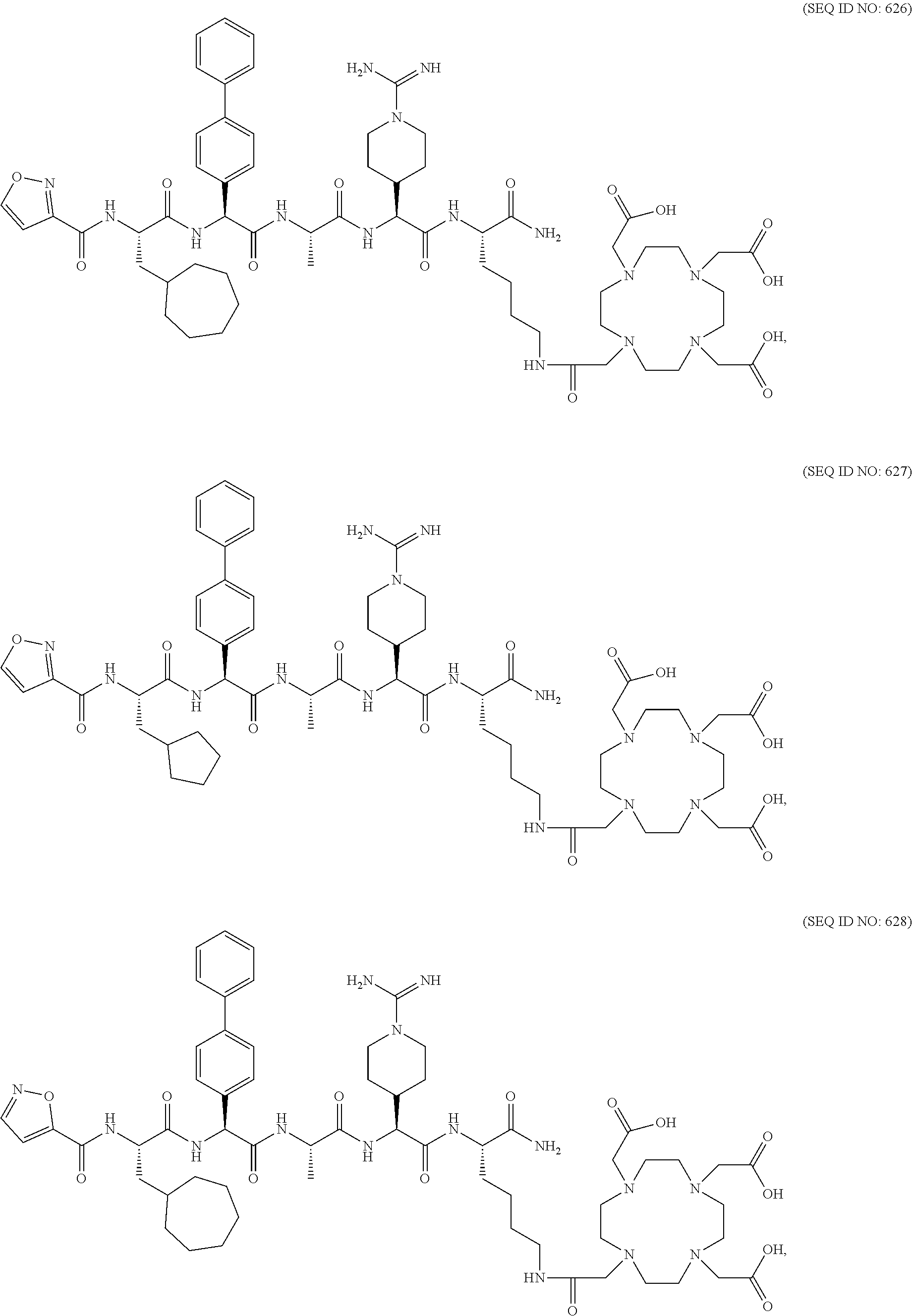

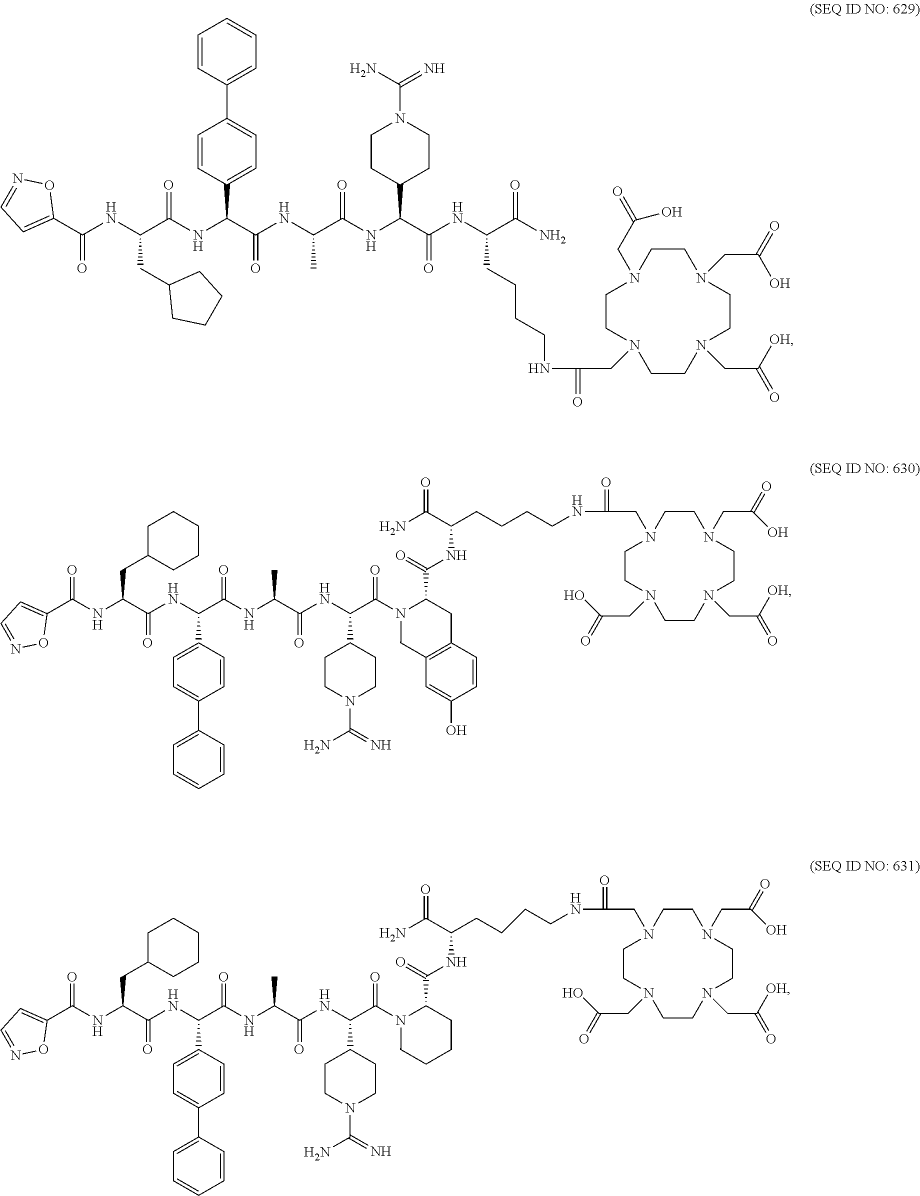

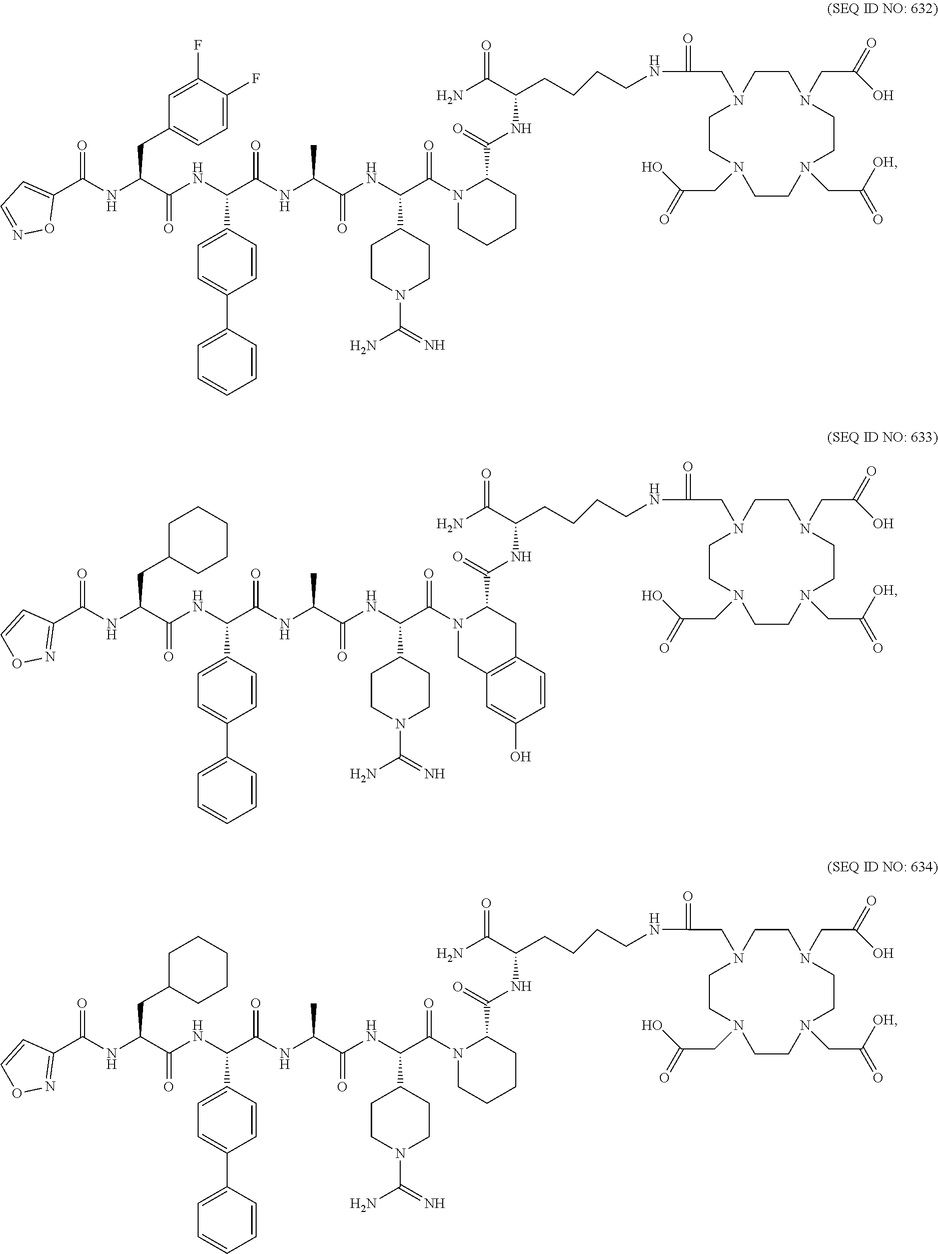

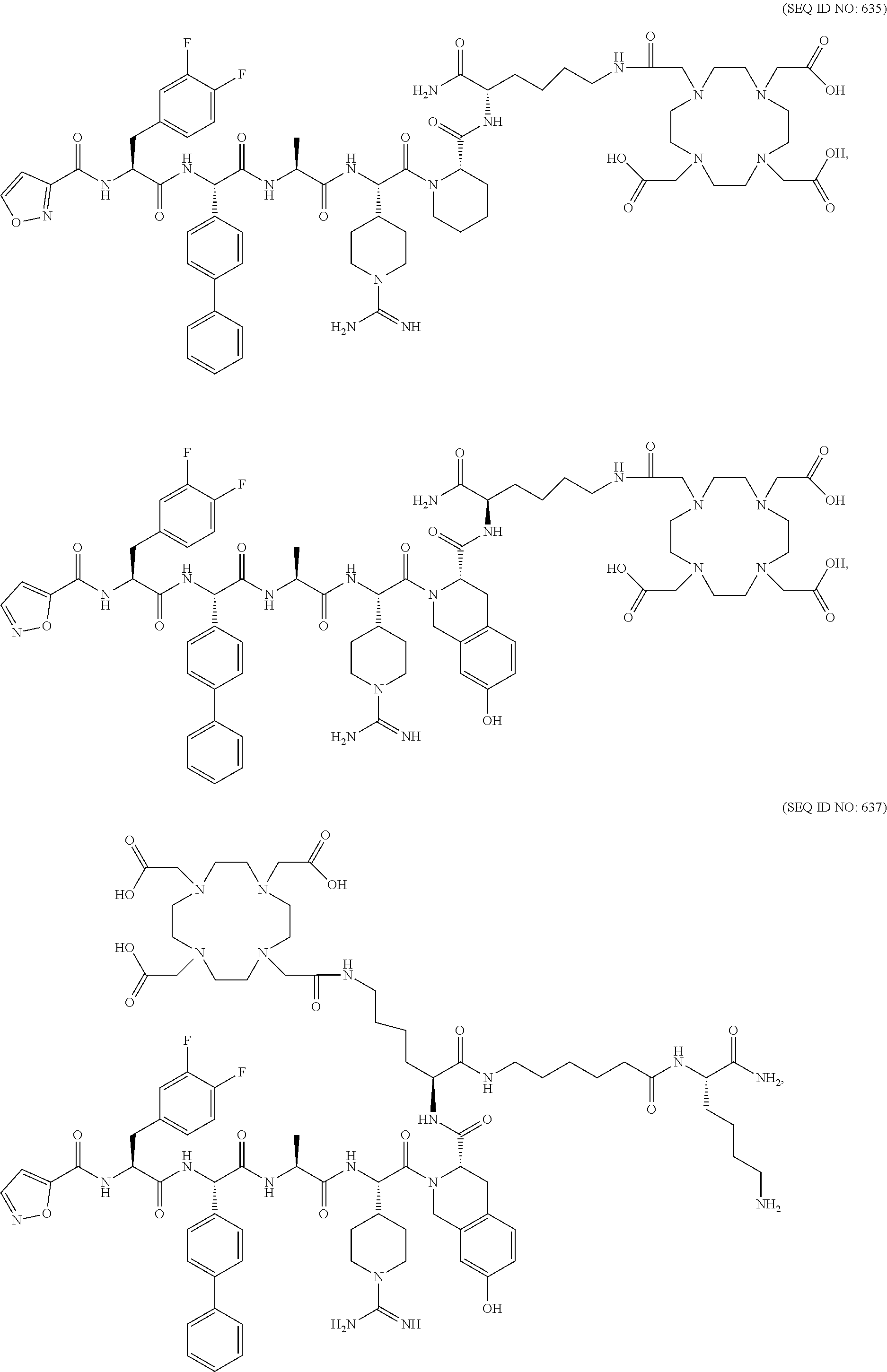

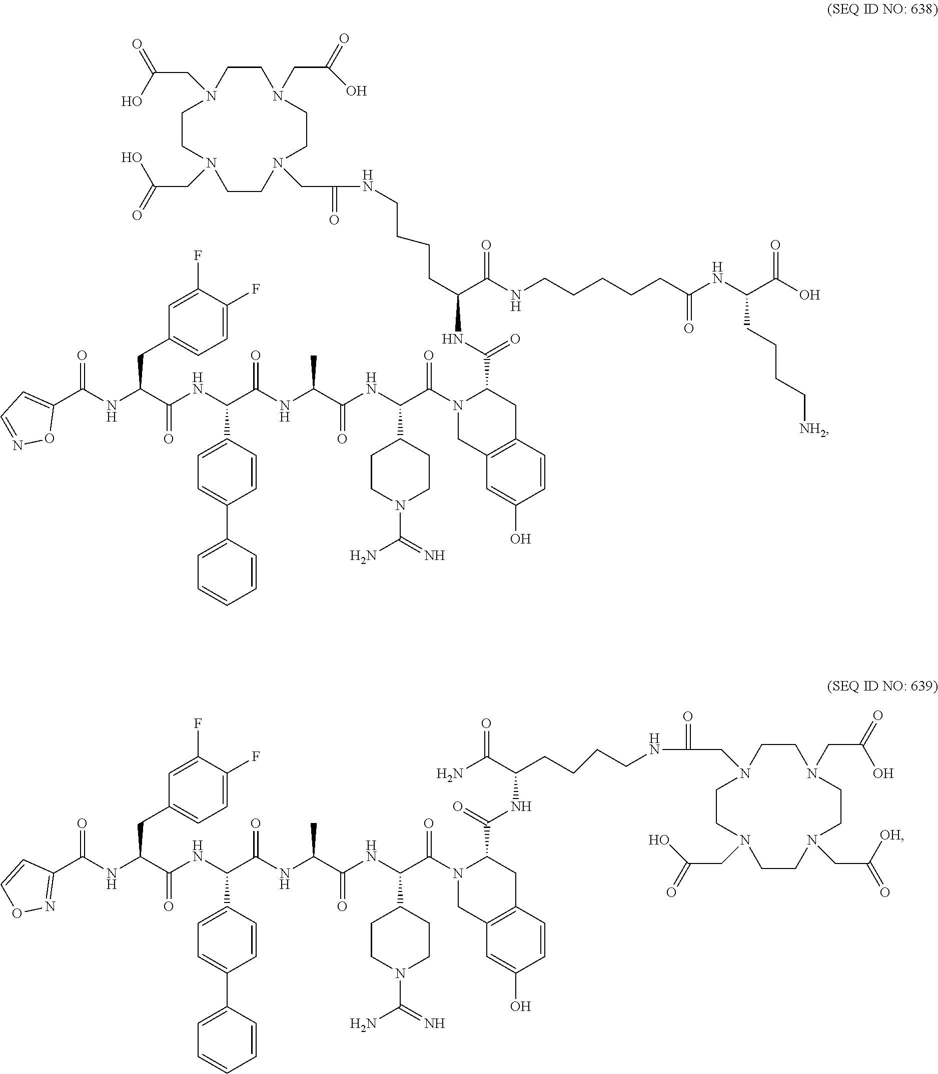 [SMILES: C[C@H](NC(=O)[C@@H](NC(=O)[C@H](Cc1ccc(F)c(F)c1)NC(=O)c1ccno1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(=O)O)C1CCN(C(=N)N)CC1 ; C[C@H](NC(=O)[C@@H](NC(=O)[C@H](Cc1ccc(F)c(F)c1)NC(=O)c1ccon1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

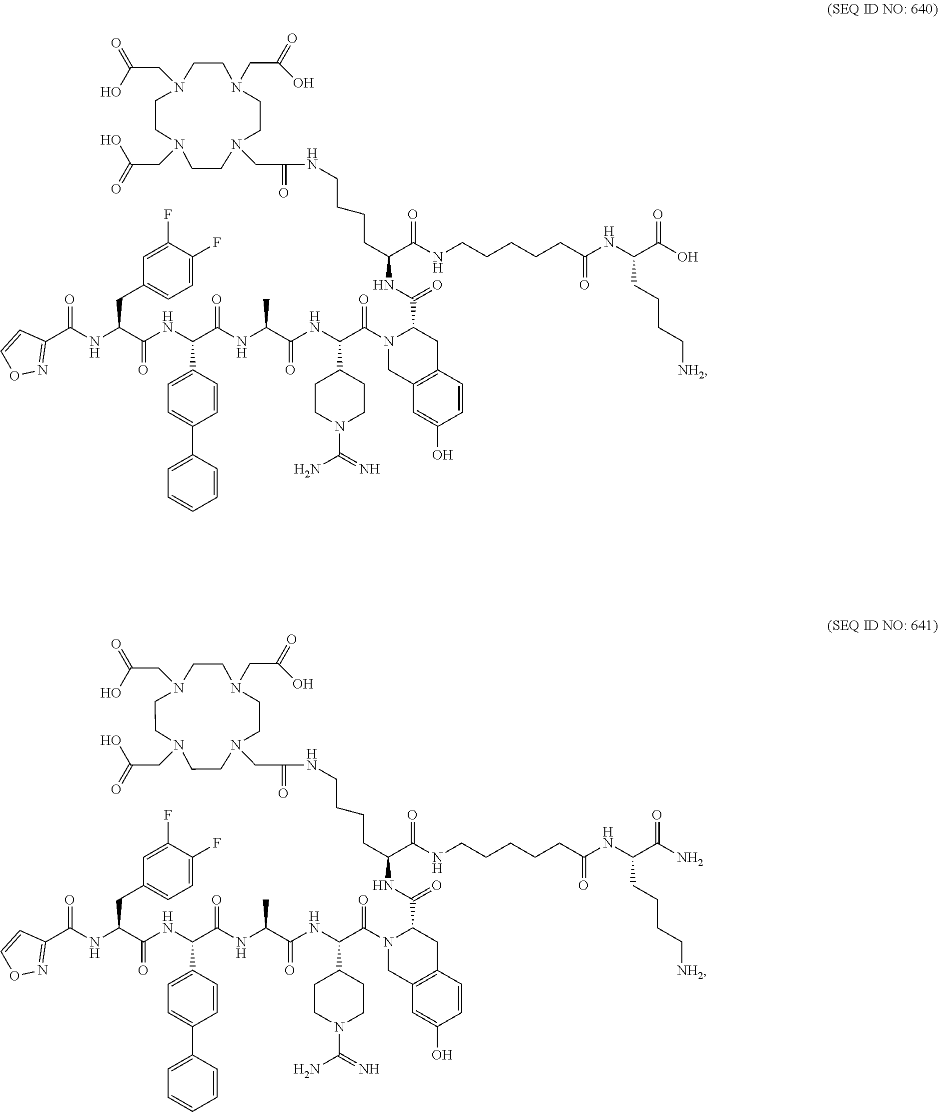 [SMILES: C[C@H](NC(=O)[C@@H](NC(=O)[C@H](Cc1ccc(F)c(F)c1)NC(=O)c1ccon1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(=O)O)C1CCN(C(=N)N)CC1 ; C[C@H](NC(=O)[C@@H](NC(=O)[C@H](Cc1ccc(F)c(F)c1)NC(=O)c1ccon1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

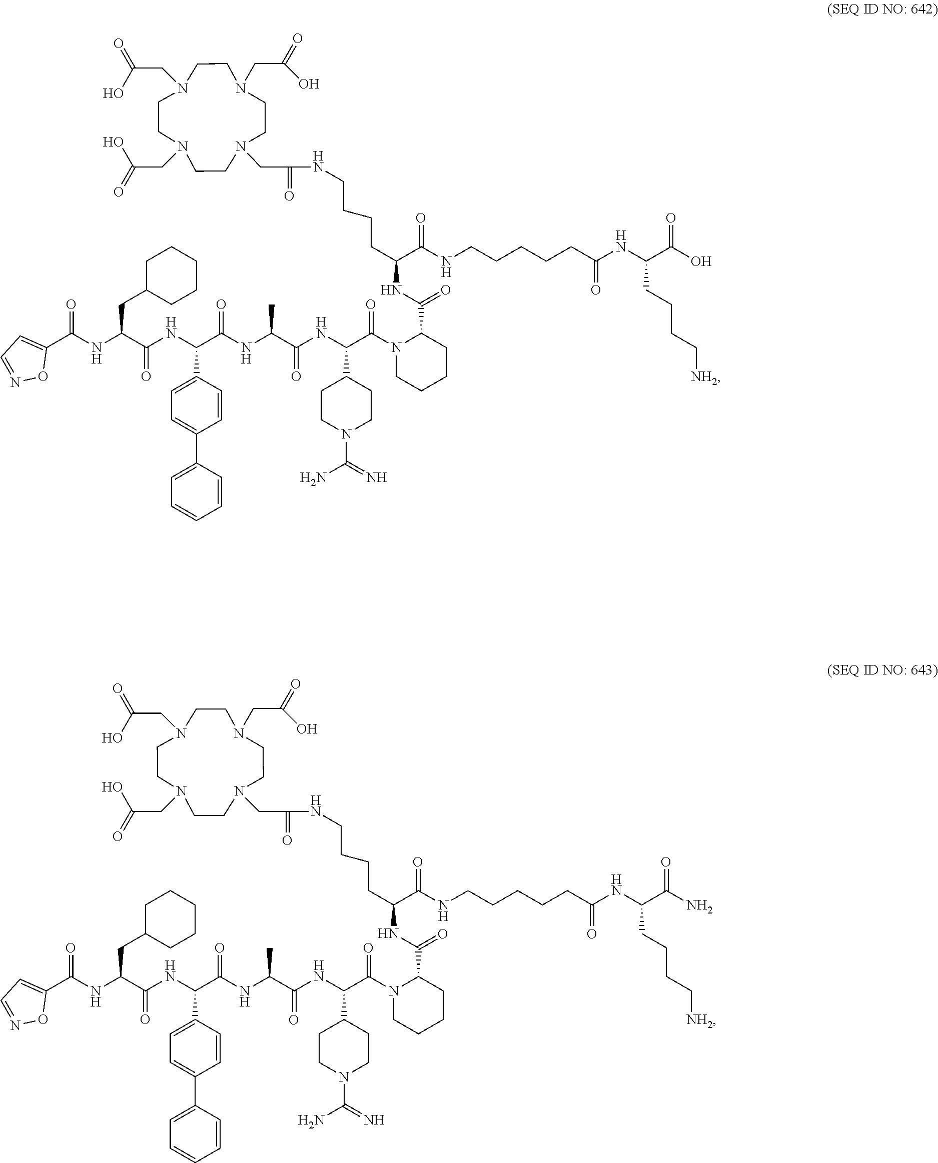 [SMILES: C[C@H](NC(=O)[C@@H](NC(=O)[C@H](CC1CCCCC1)NC(=O)c1ccno1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(=O)O)C1CCN(C(=N)N)CC1 ; C[C@H](NC(=O)[C@@H](NC(=O)[C@H](CC1CCCCC1)NC(=O)c1ccno1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

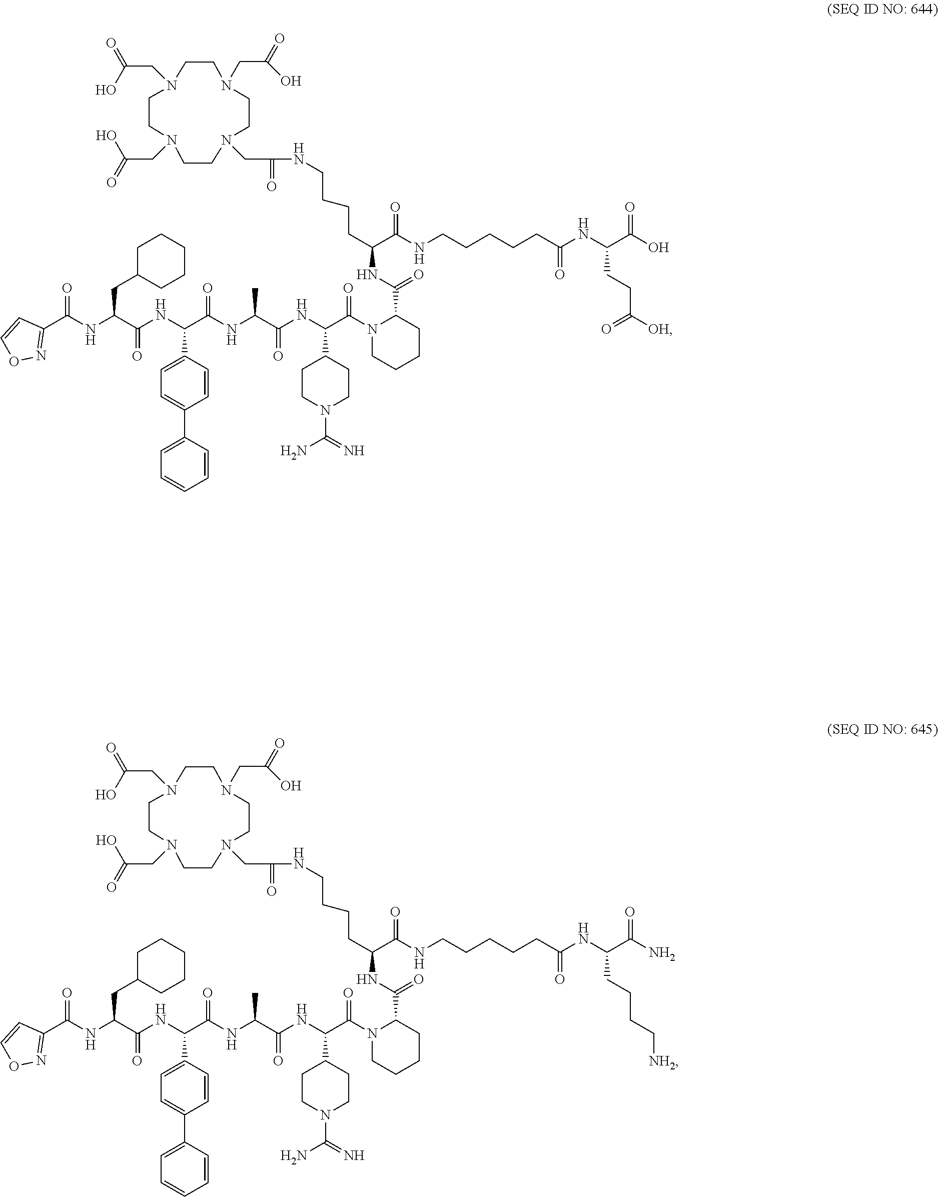 [SMILES: C[C@H](NC(=O)[C@@H](NC(=O)[C@H](CC1CCCCC1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCC(=O)O)C(=O)O)C1CCN(C(=N)N)CC1 ; C[C@H](NC(=O)[C@@H](NC(=O)[C@H](CC1CCCCC1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

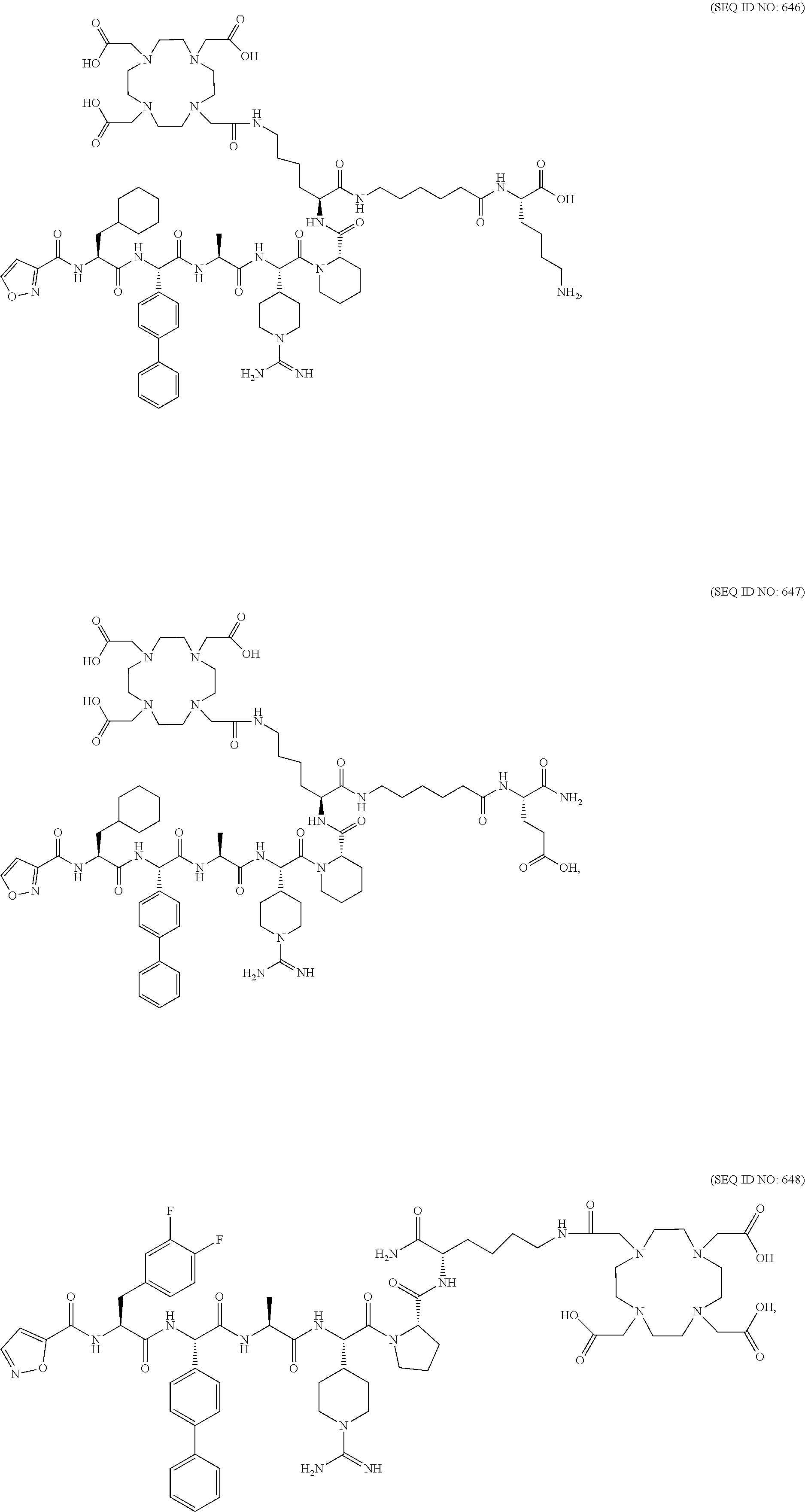

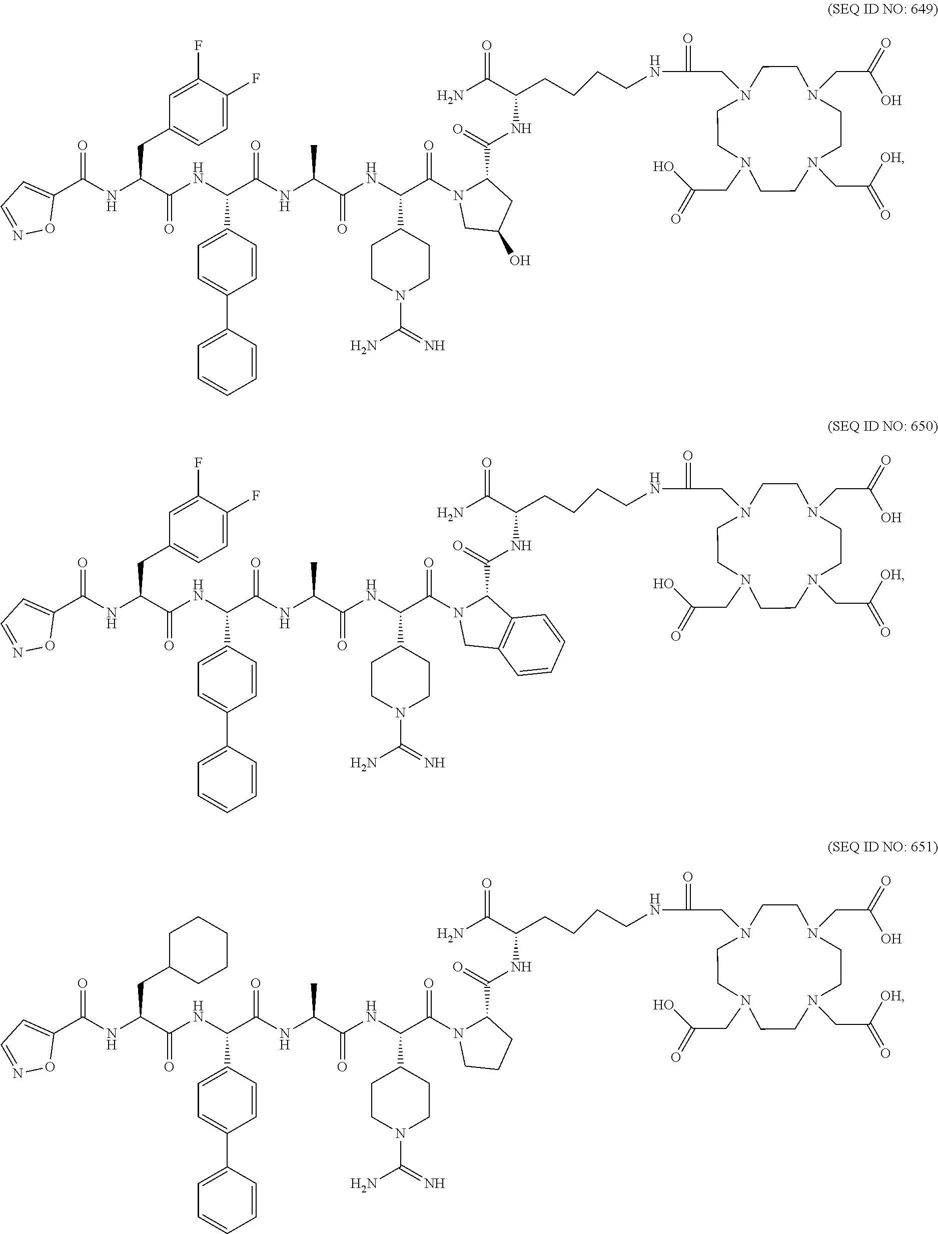

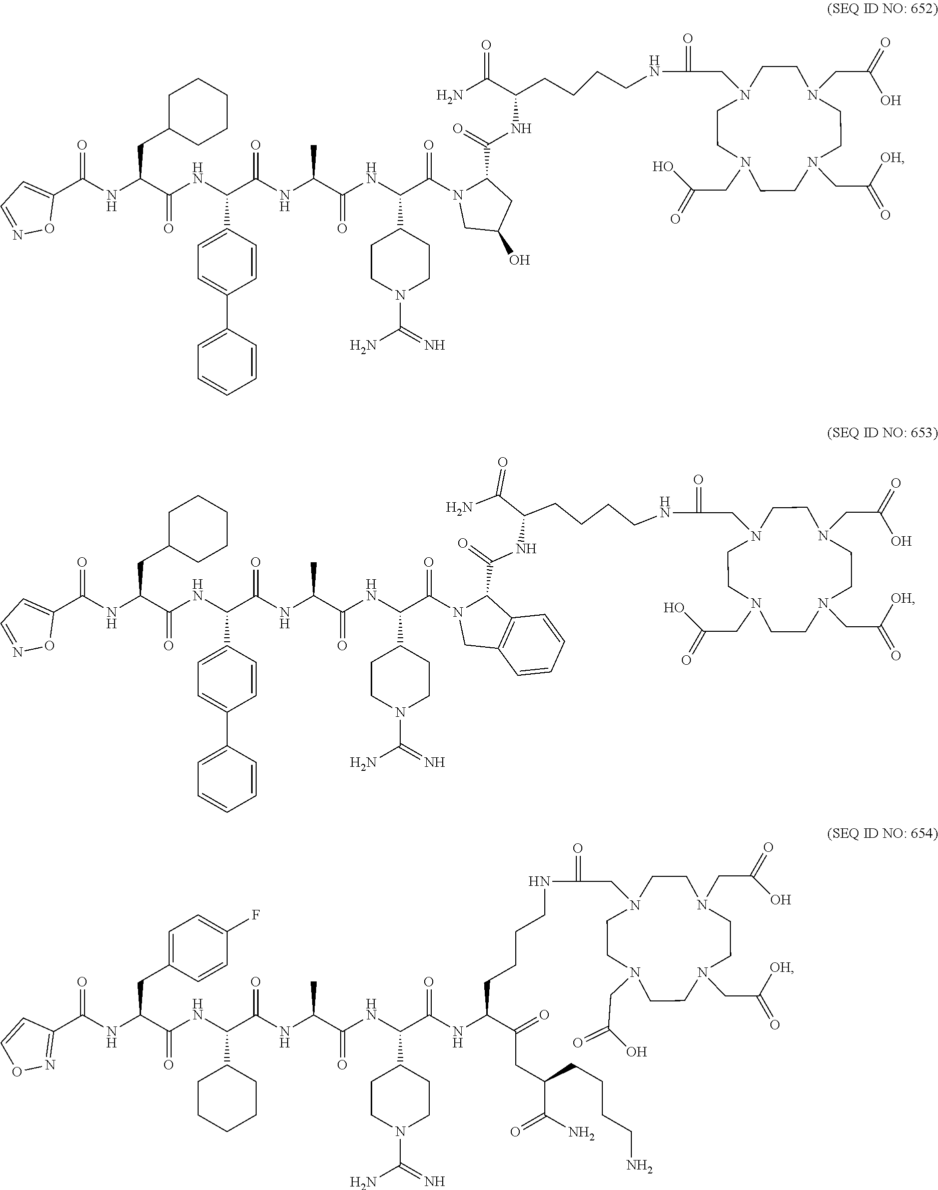

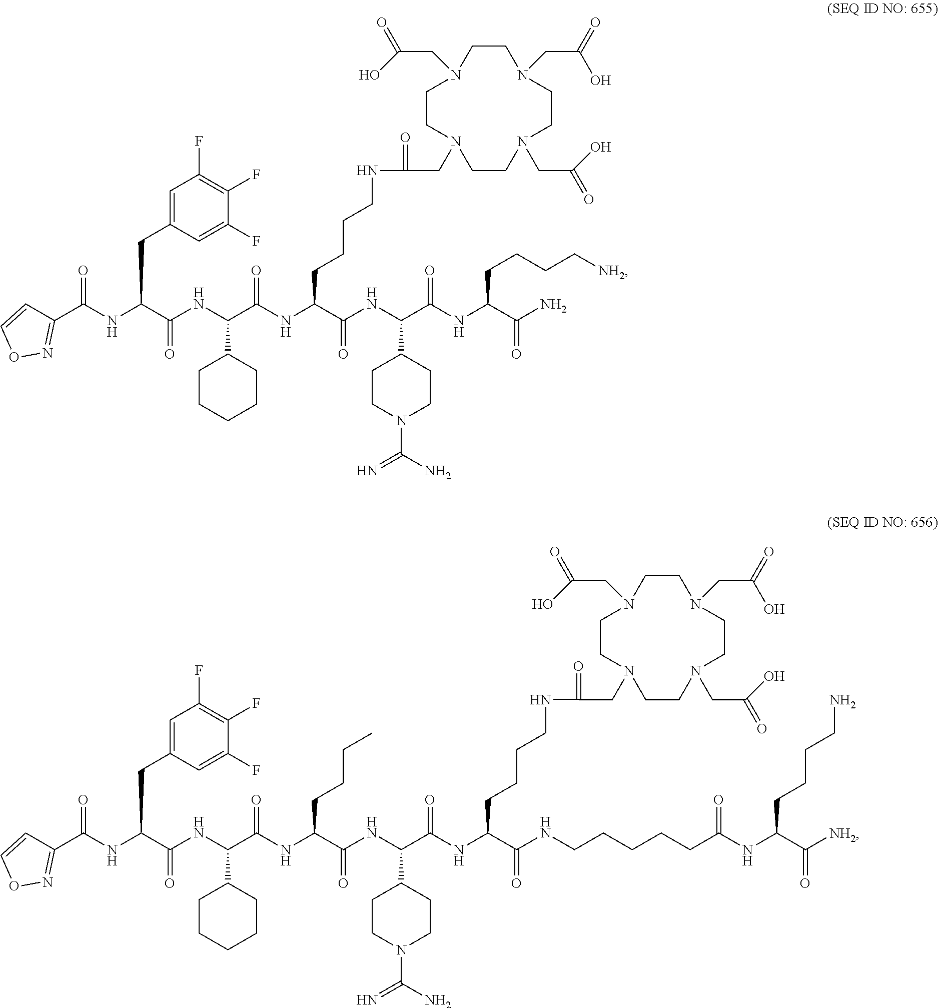 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@H](Cc2cc(F)c(F)c(F)c2)NC(=O)c2ccon2)C2CCCCC2)C(=O)N[C@@H](CCCCN)C(N)=O)CC1 ; CCCC[C@H](NC(=O)[C@@H](NC(=O)[C@H](Cc1cc(F)c(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

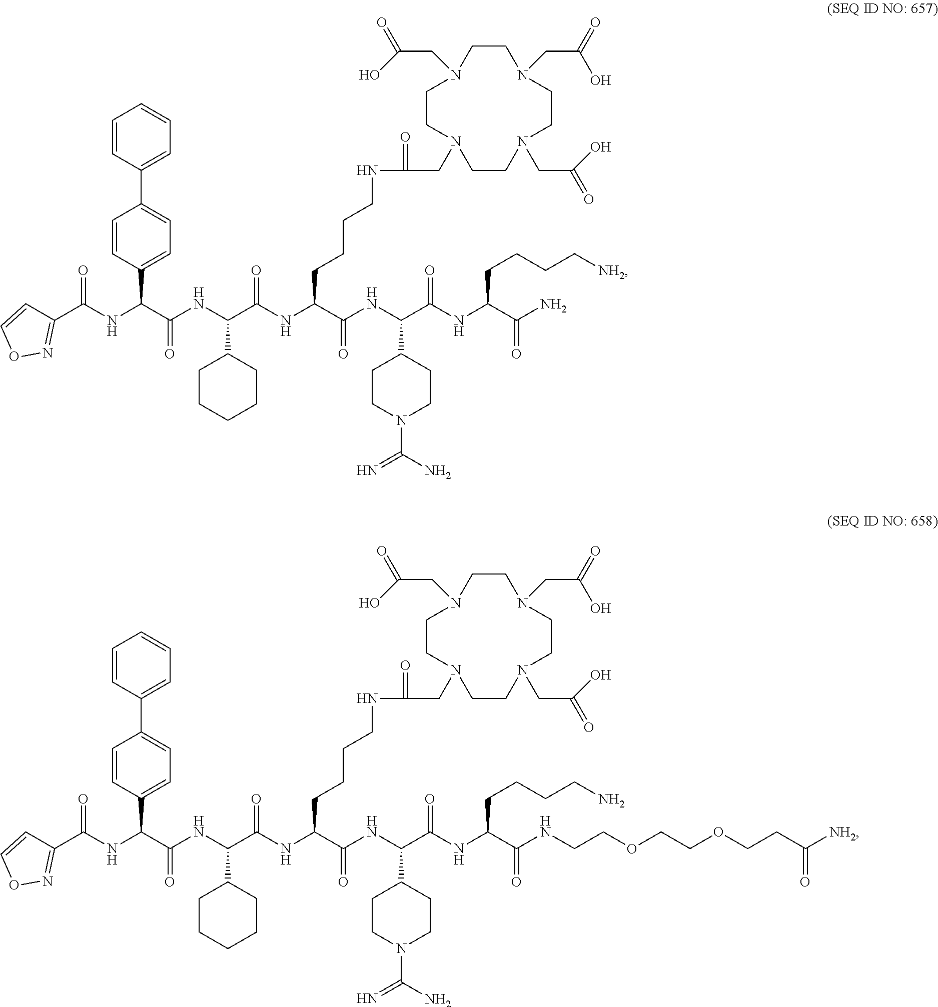 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@@H](NC(=O)c2ccon2)c2ccc(-c3ccccc3)cc2)C2CCCCC2)C(=O)N[C@@H](CCCCN)C(N)=O)CC1 ; N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@@H](NC(=O)c2ccon2)c2ccc(-c3ccccc3)cc2)C2CCCCC2)C(=O)N[C@@H](CCCCN)C(=O)NCCOCCOCCC(N)=O)CC1]

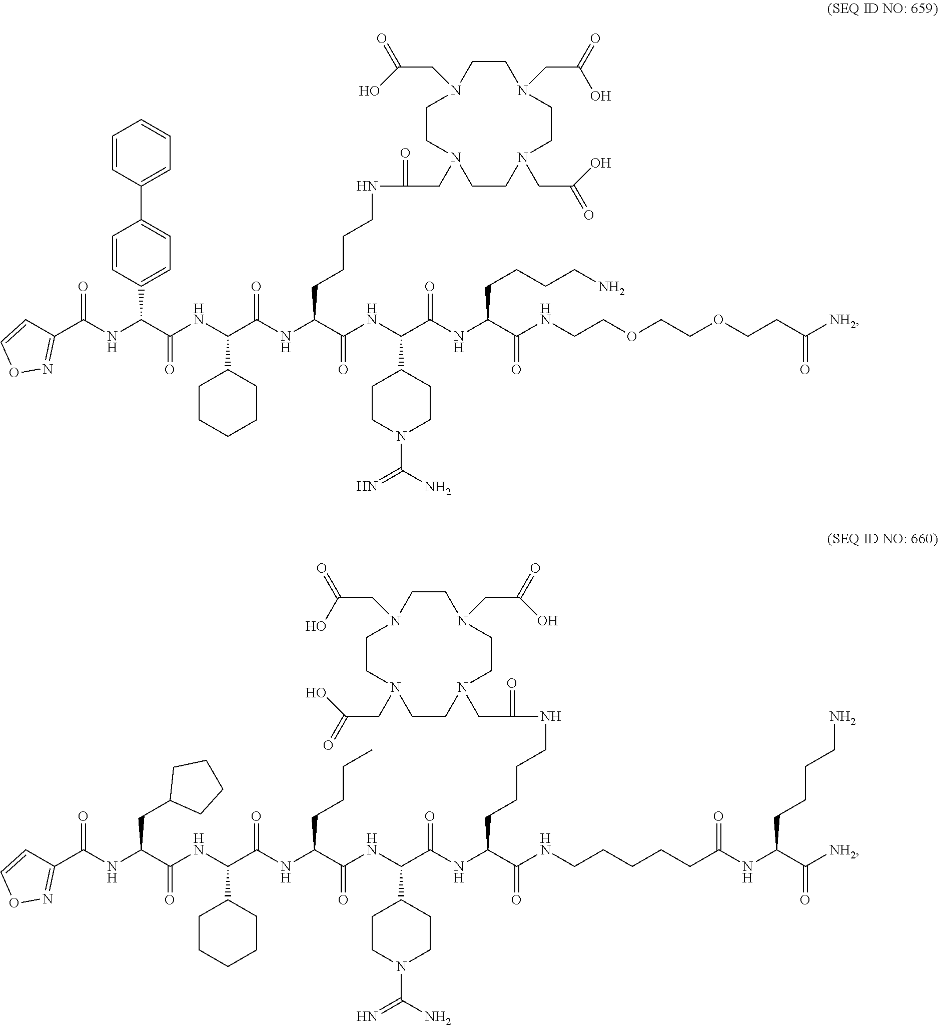 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@H](NC(=O)c2ccon2)c2ccc(-c3ccccc3)cc2)C2CCCCC2)C(=O)N[C@@H](CCCCN)C(=O)NCCOCCOCCC(N)=O)CC1 ; CCCC[C@H](NC(=O)[C@@H](NC(=O)[C@H](CC1CCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

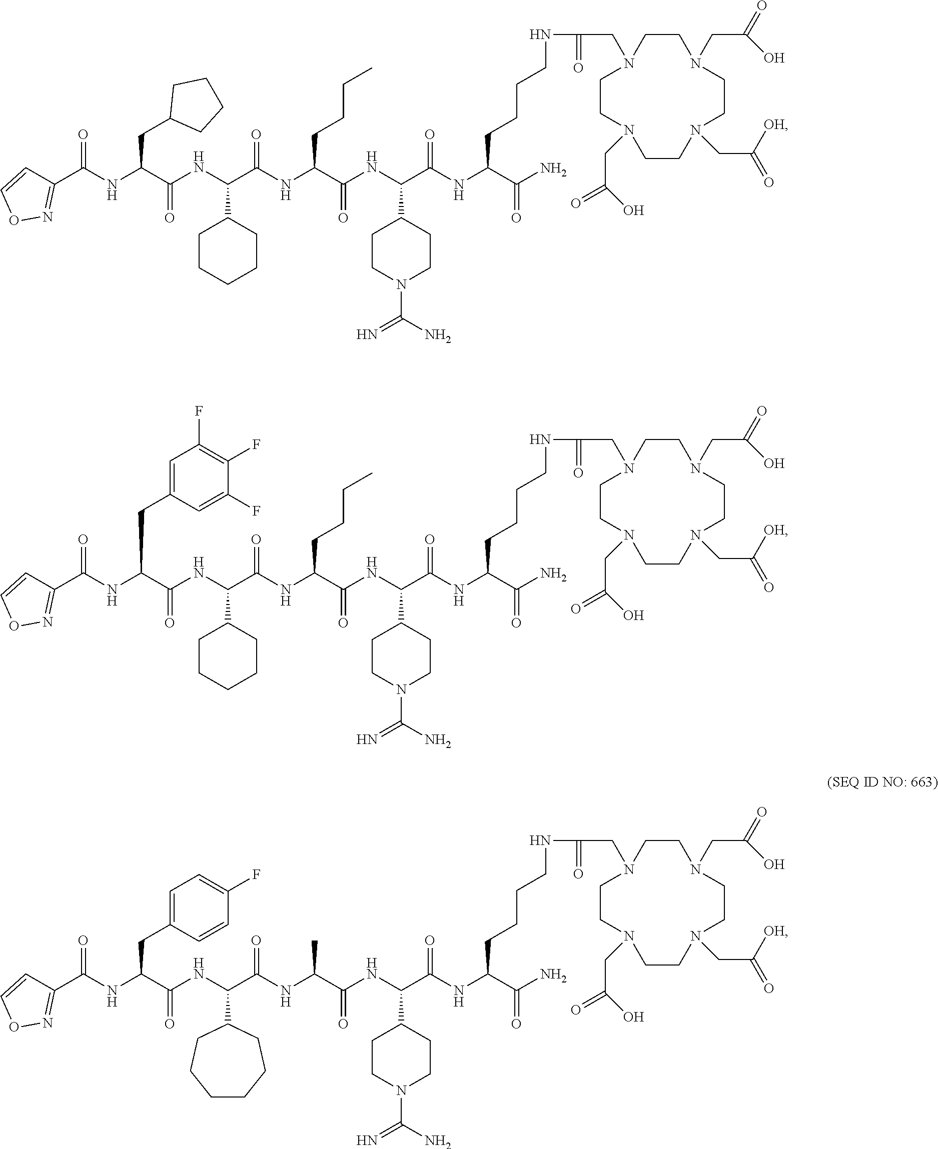

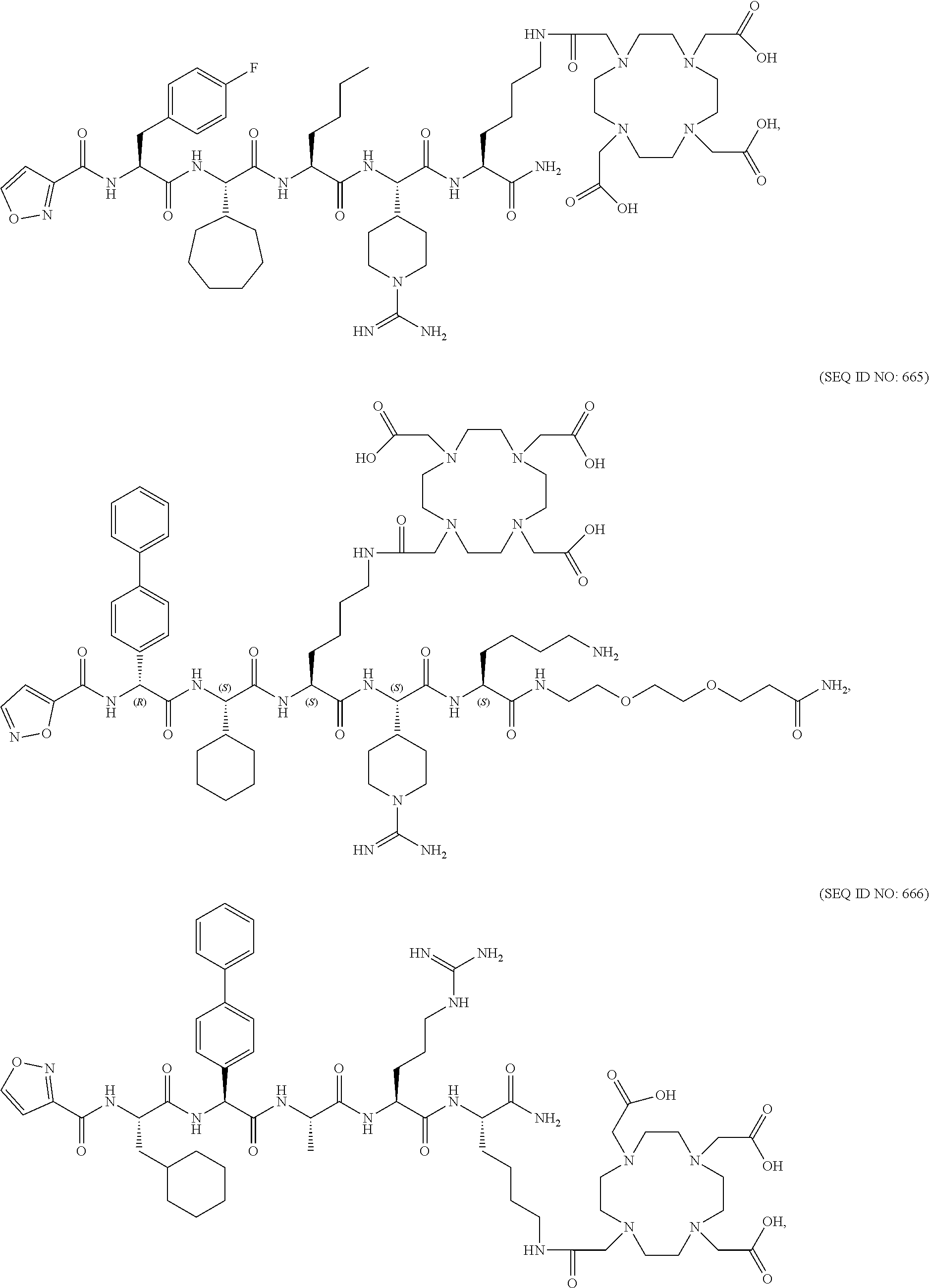

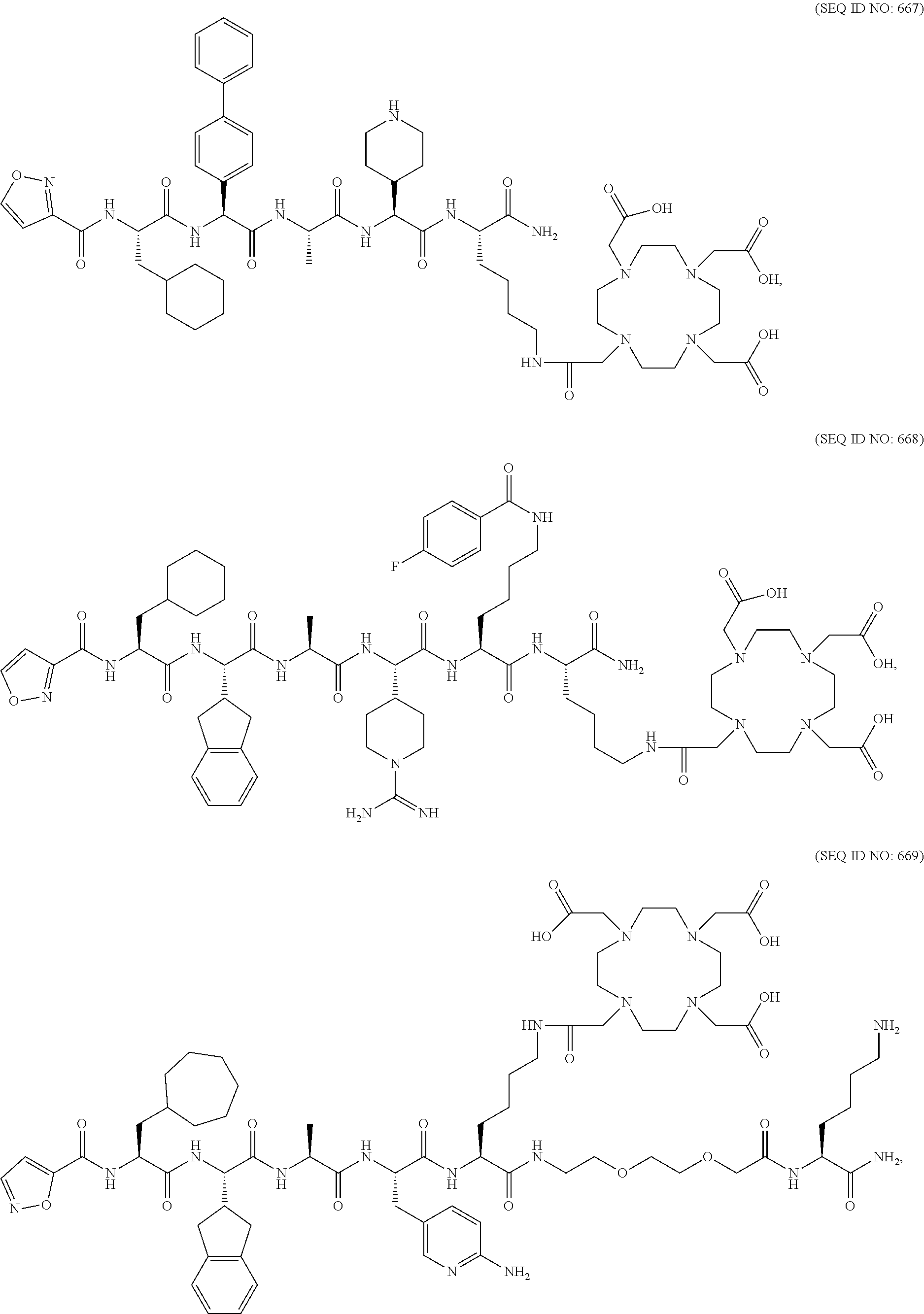

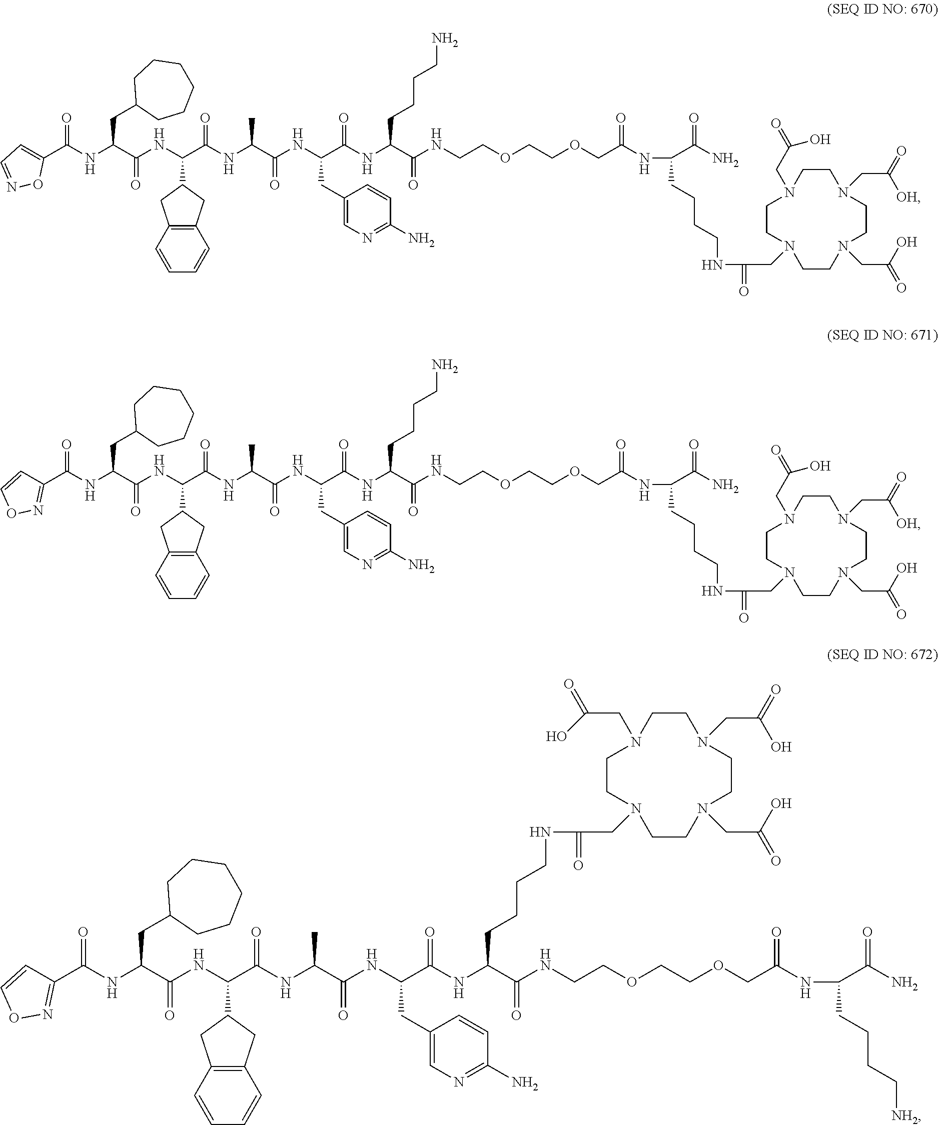

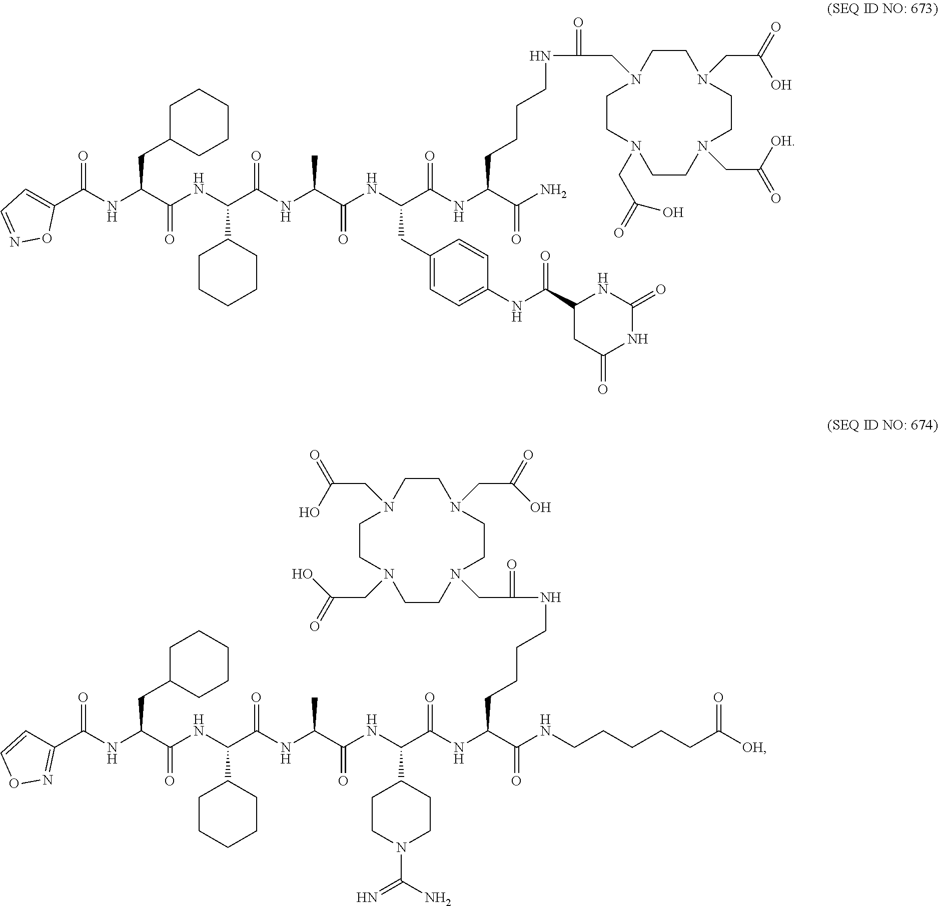

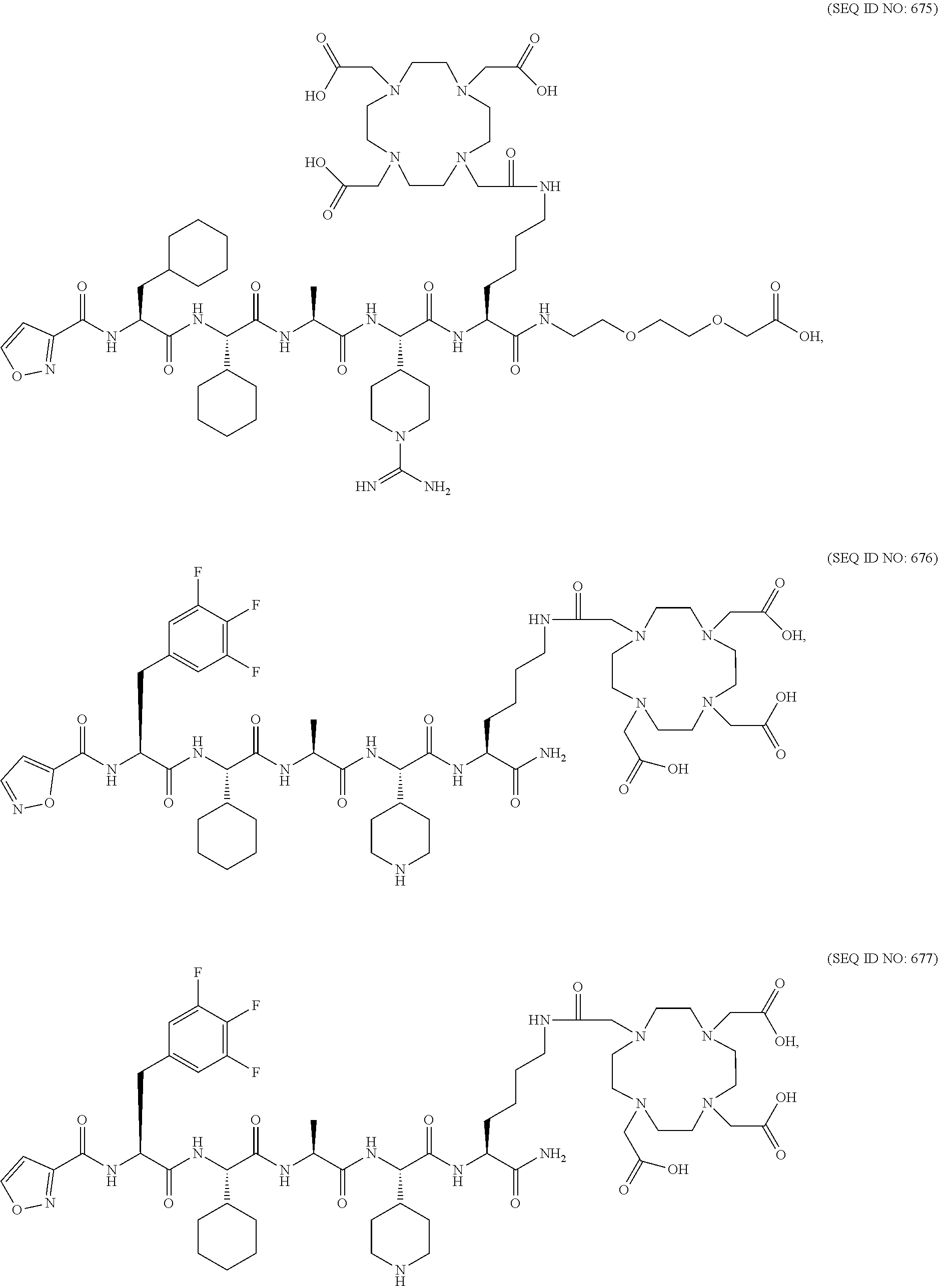

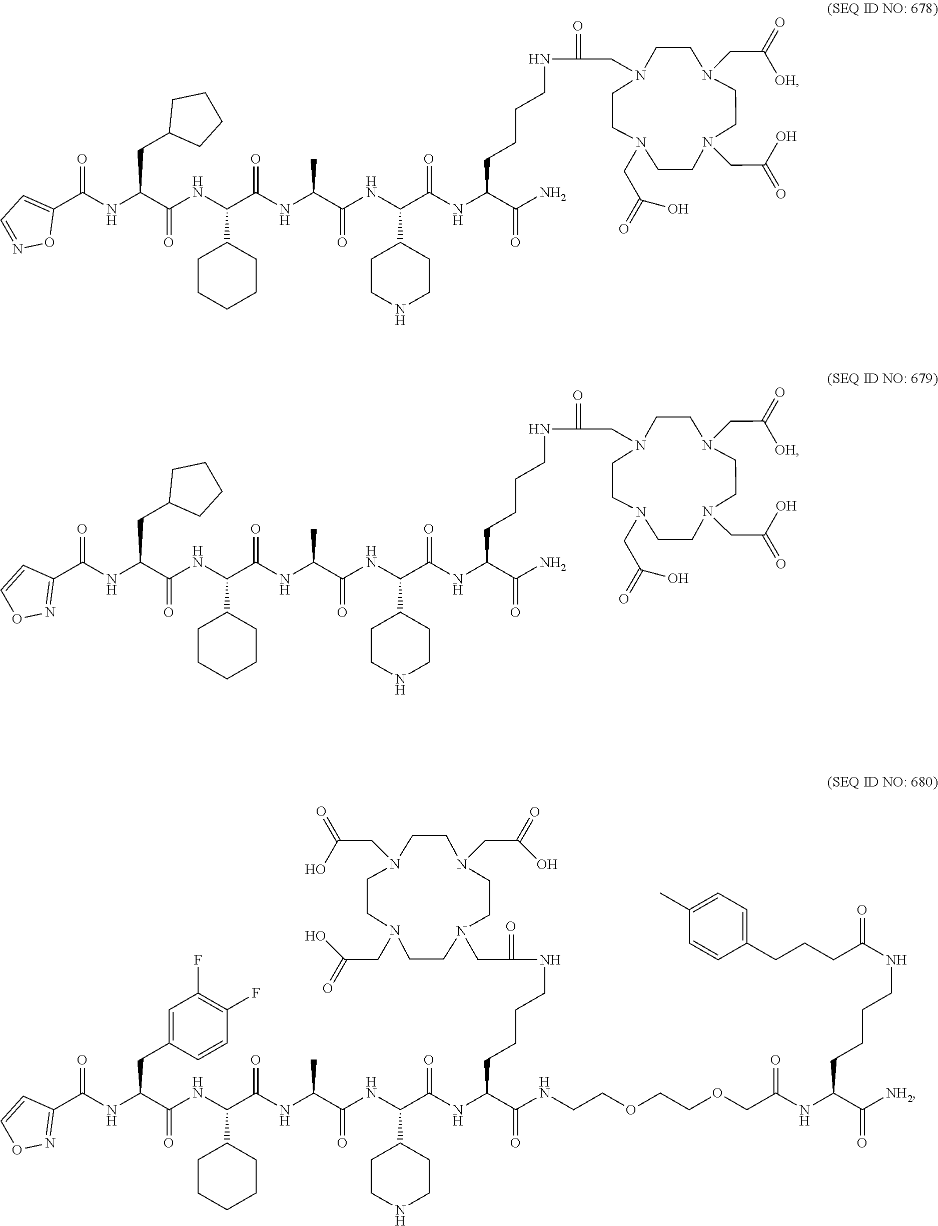

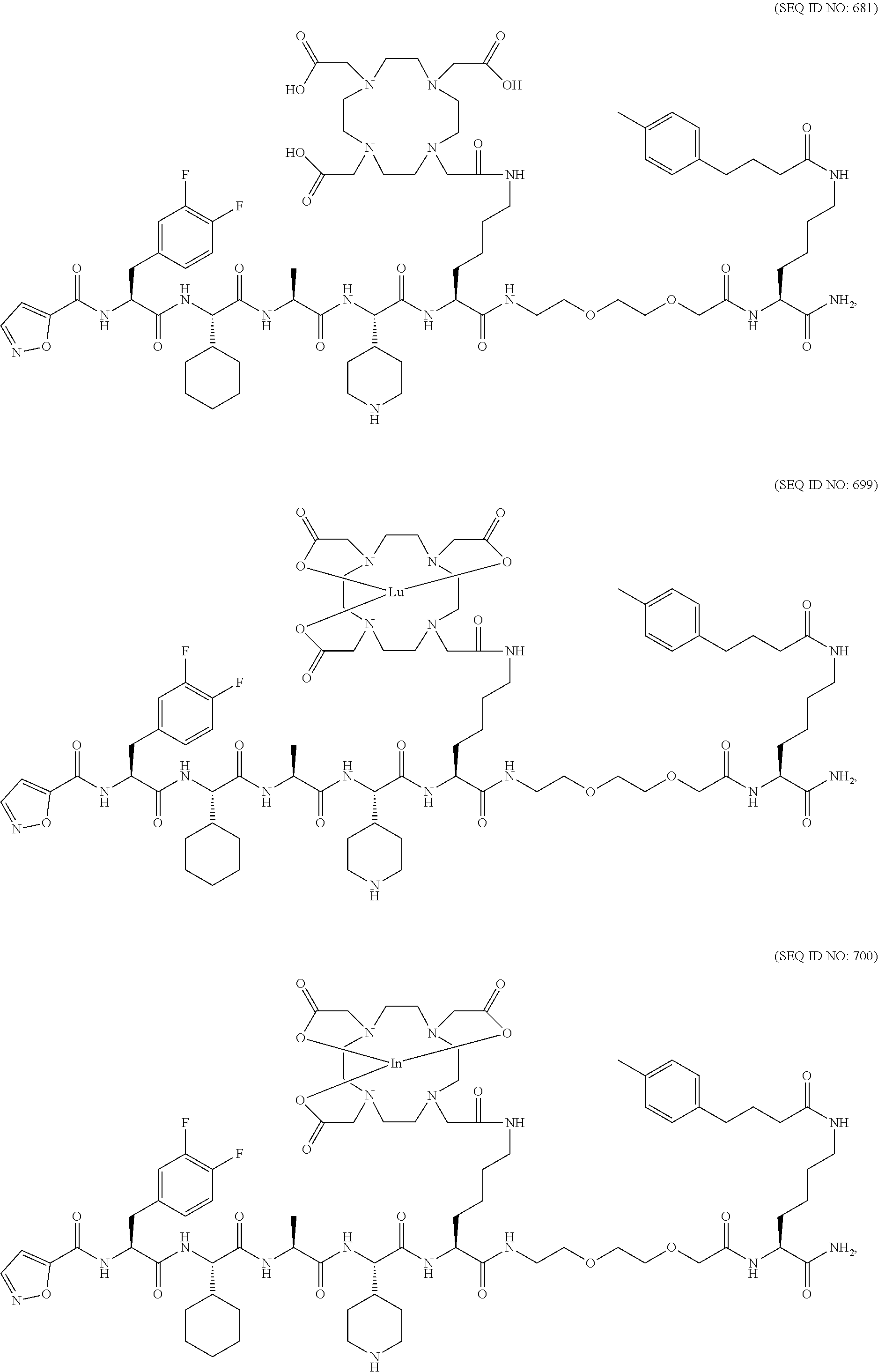

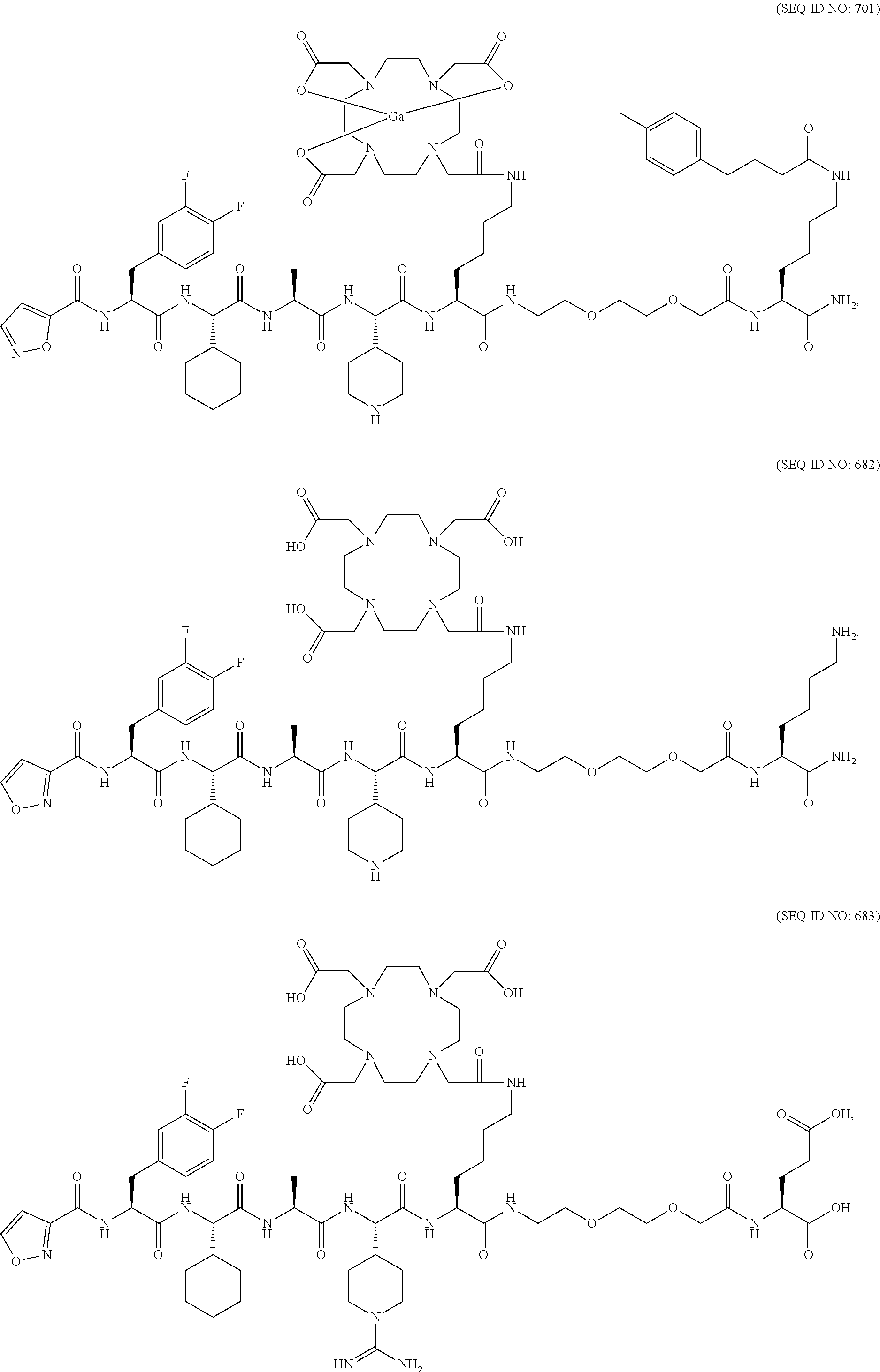

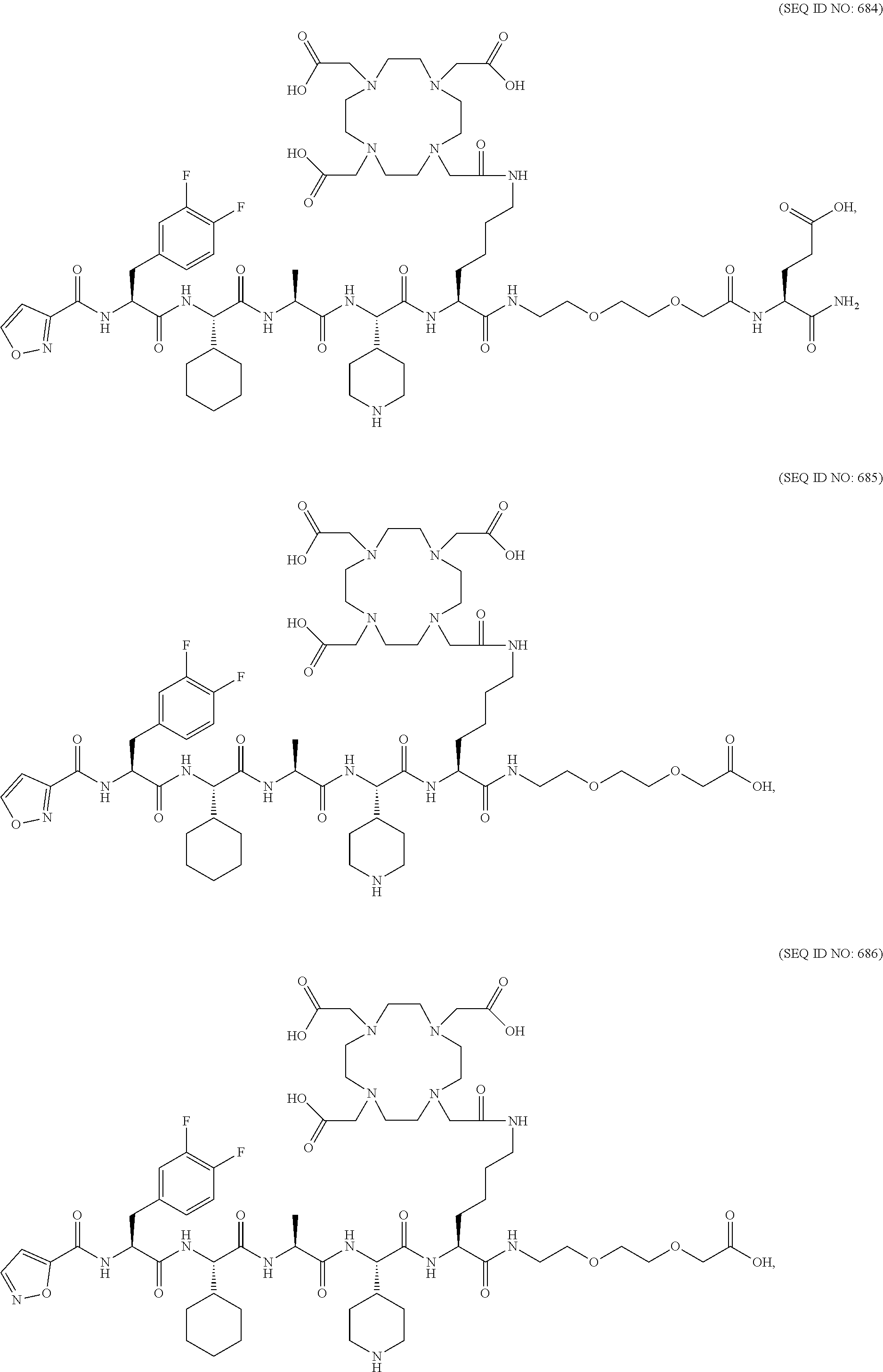

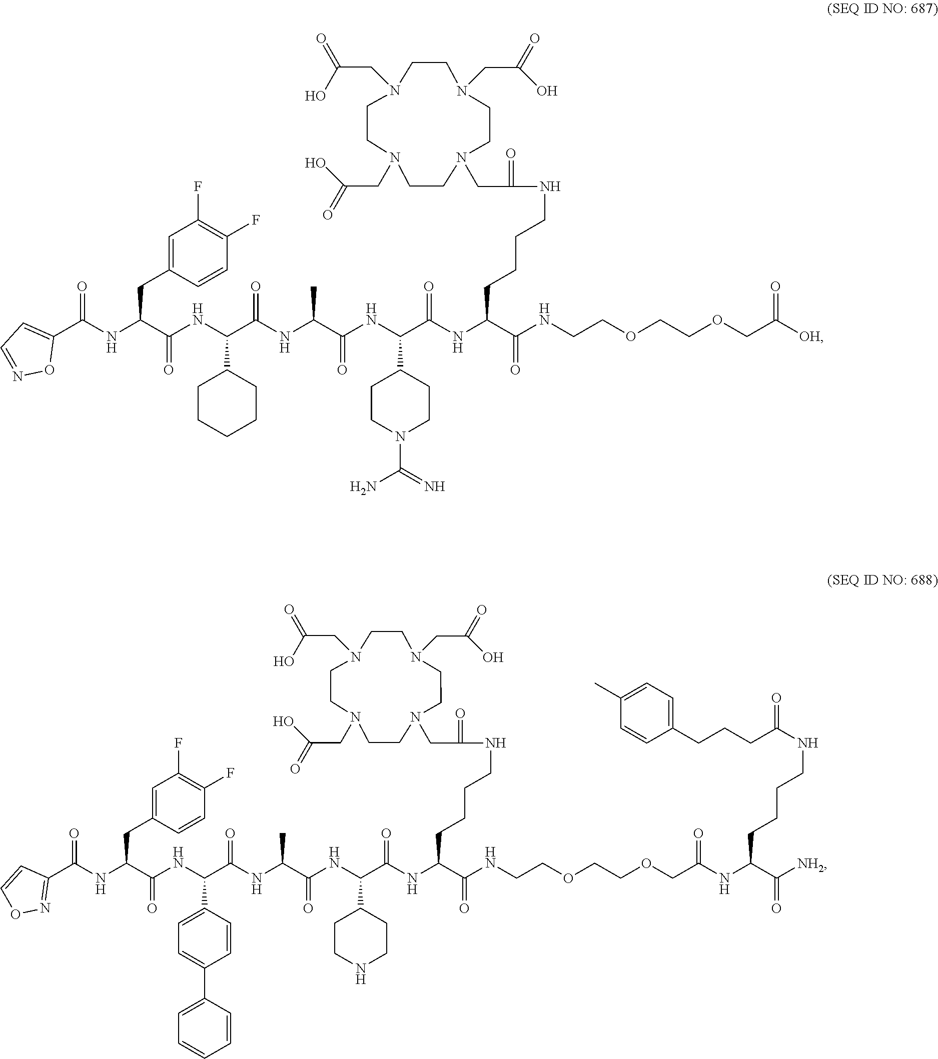 [SMILES: C[C@H](NC(=O)[C@@H](NC(=O)[C@H](Cc1ccc(F)c(F)c1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)O)C1CCN(C(=N)N)CC1 ; Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)c2ccc(-c3ccccc3)cc2)C2CCNCC2)C(N)=O)cc1]

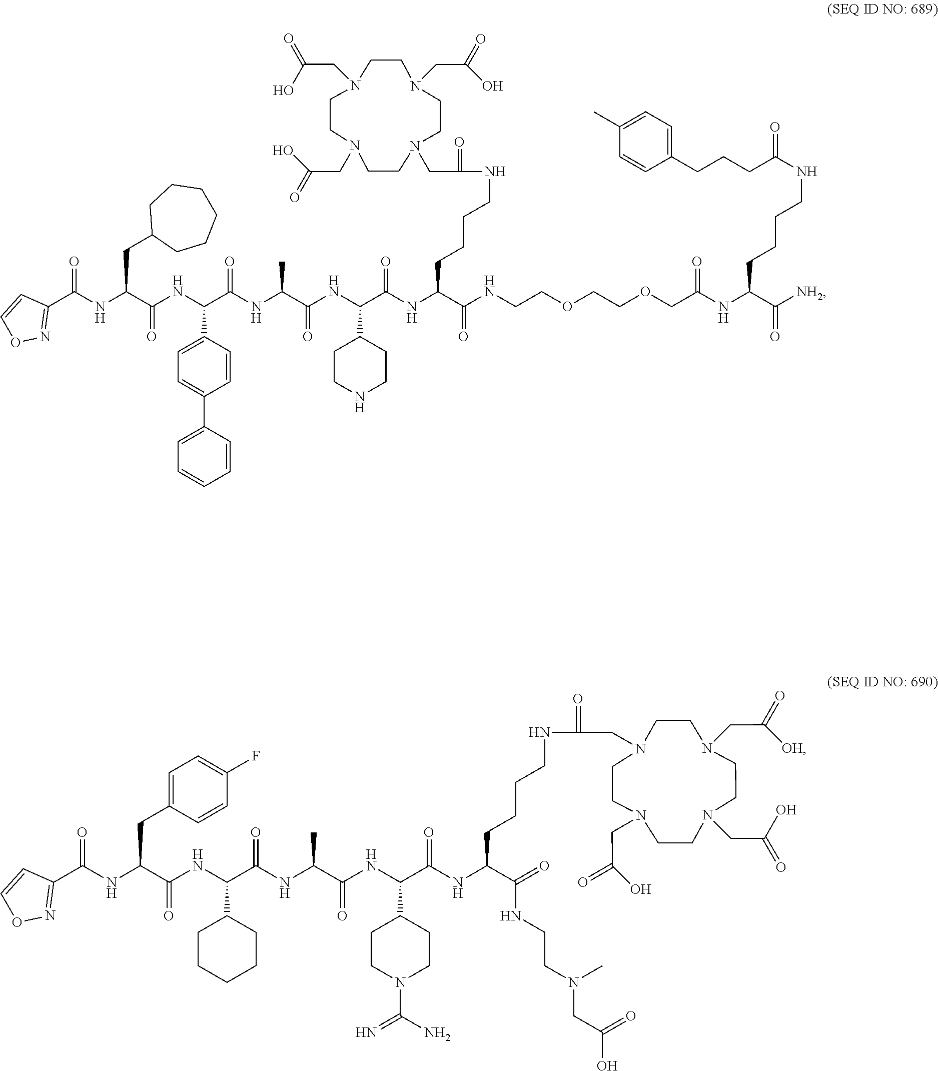 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](CC2CCCCCC2)NC(=O)c2ccon2)c2ccc(-c3ccccc3)cc2)C2CCNCC2)C(N)=O)cc1 ; C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCN(C)CC(=O)O)C1CCN(C(=N)N)CC1]

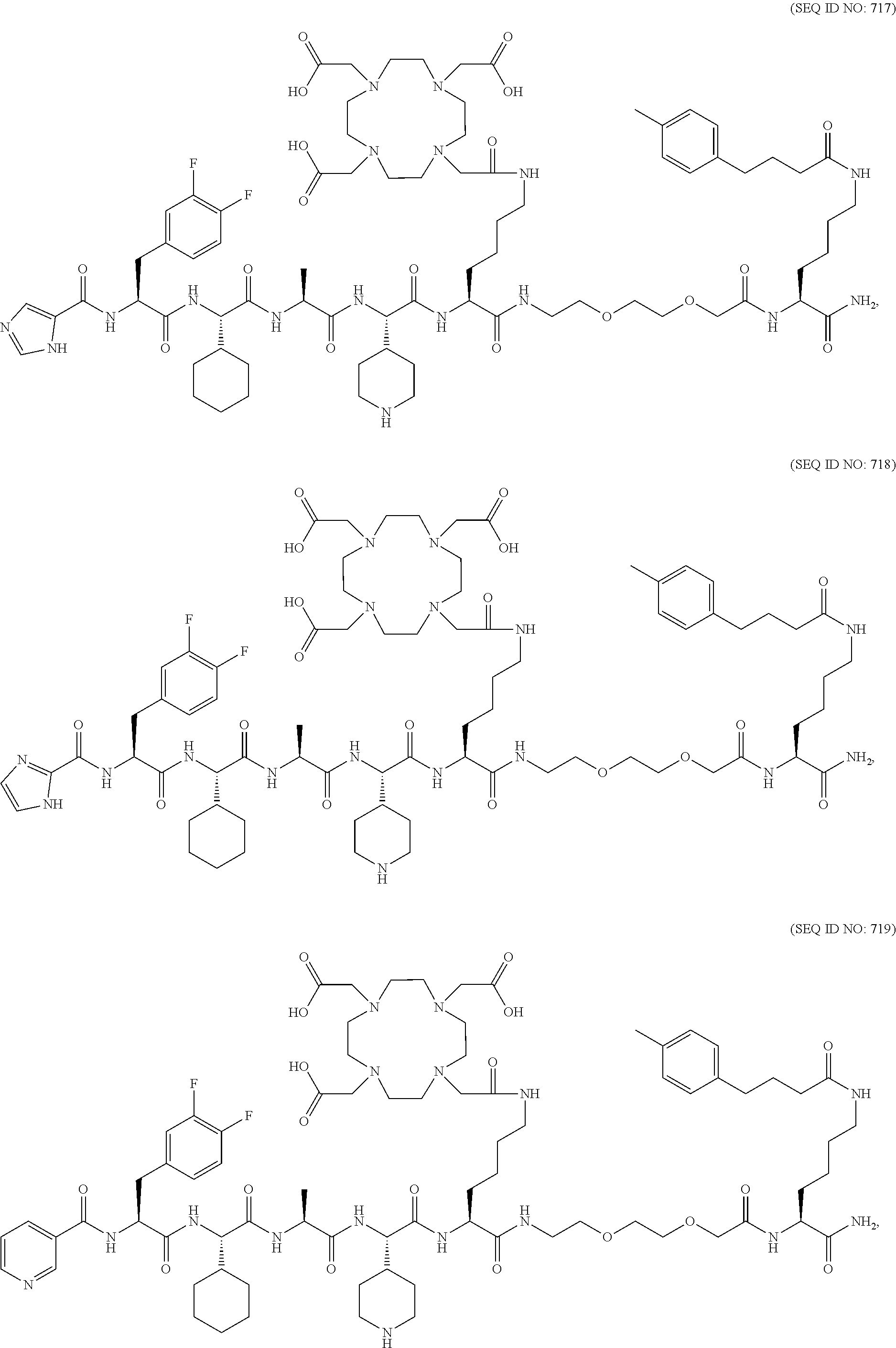

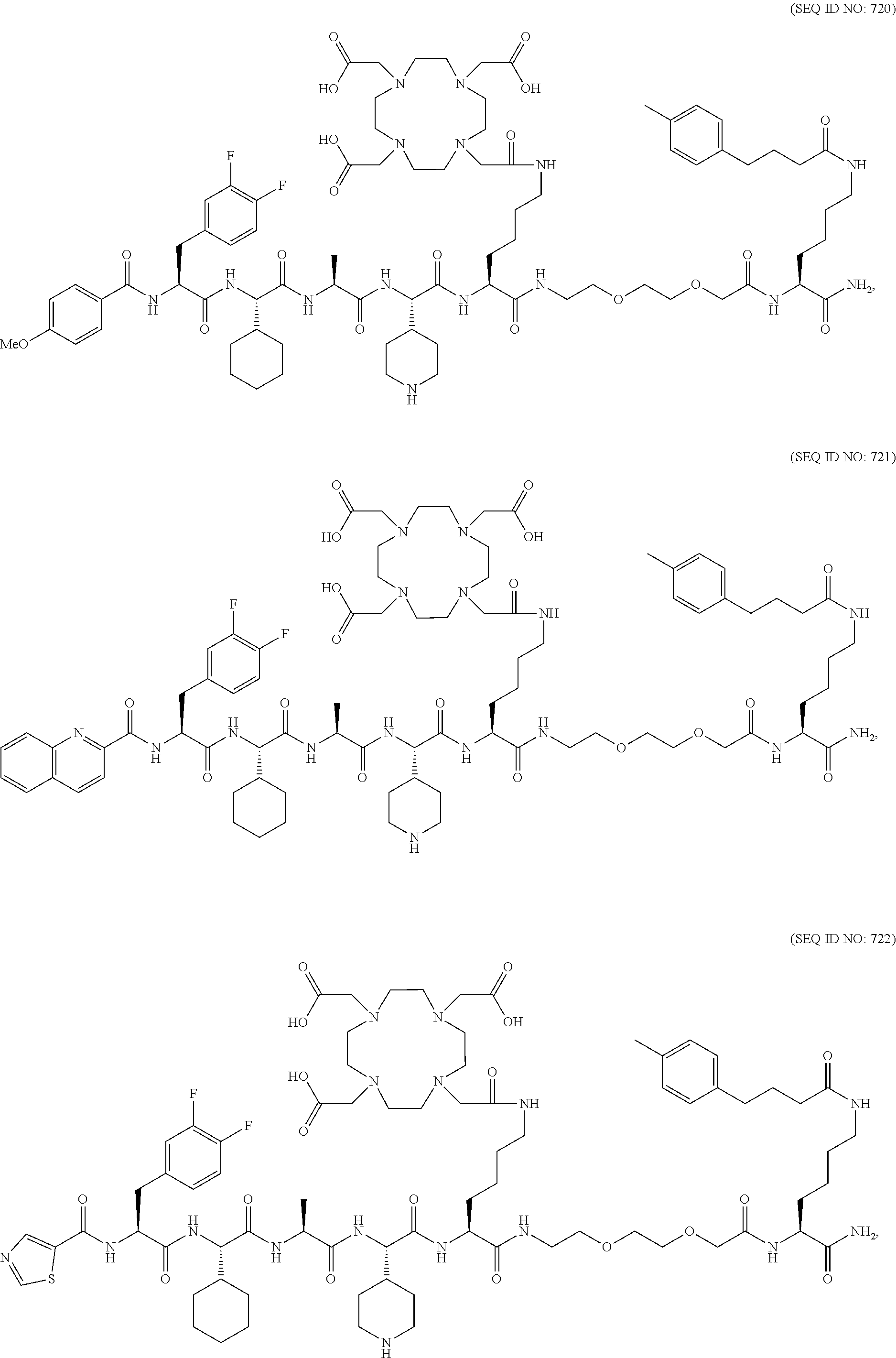

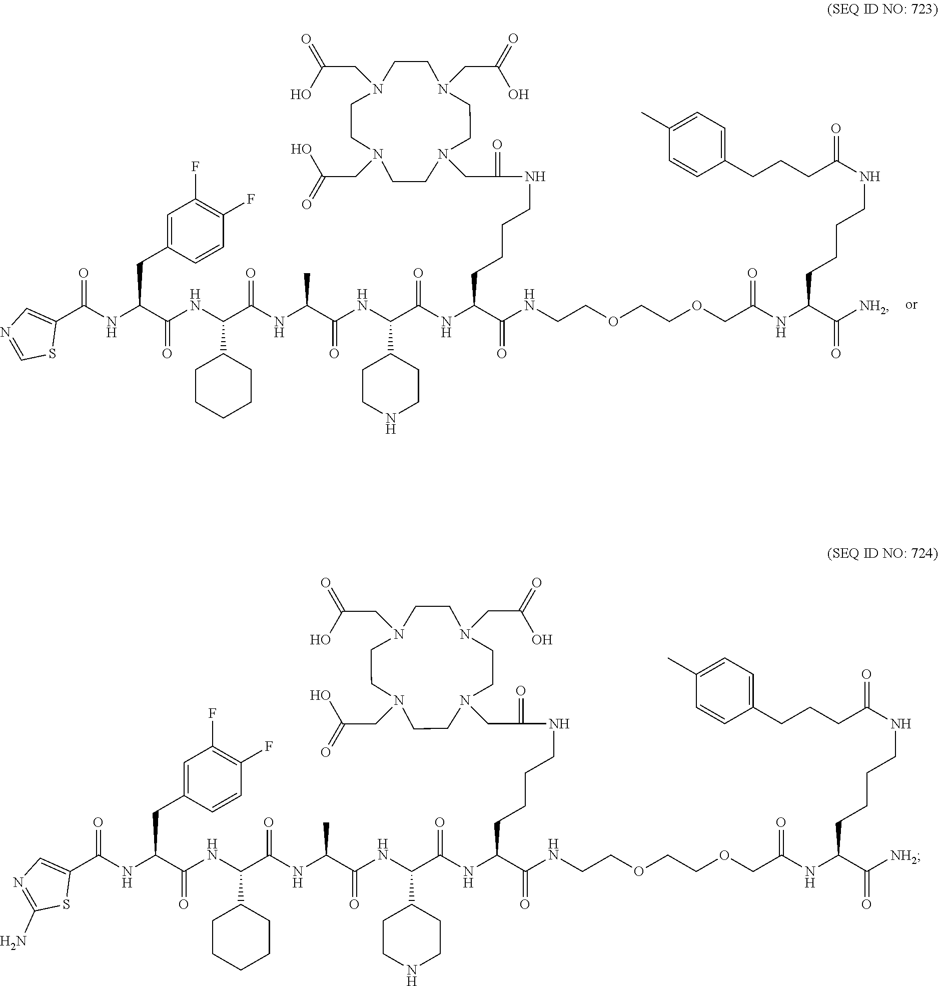 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cncs2)C2CCCCC2)C2CCNCC2)C(N)=O)cc1 ; "or" ; Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cnc(N)s2)C2CCCCC2)C2CCNCC2)C(N)=O)cc1]

or a radionuclide complex thereof.

Embodiment 73. The compound of any one of embodiments 1-72, or a pharmaceutically acceptable salt thereof, wherein: the radionuclide of the radionuclide complex is a lanthanide or an actinide.

Embodiment 74. The compound of any one of embodiments 1-72, or a pharmaceutically acceptable salt thereof, wherein: the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

Embodiment 75. The compound of any one of embodiments 1-72, or a pharmaceutically acceptable salt thereof, wherein: the radionuclide of the radionuclide complex is a diagnostic or therapeutic radionuclide.

Embodiment 76. The compound of any one of embodiments 1-72, or a pharmaceutically acceptable salt thereof, wherein: the radionuclide of the radionuclide complex is an Auger electron-emitting radionuclide, α-emitting radionuclide, β-emitting radionuclide, or γ-emitting radionuclide.

Embodiment 77. The compound of any one of embodiments 1-72, or a pharmaceutically acceptable salt thereof, wherein the radionuclide of the radionuclide complex is:
an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt) or
an α-emitting radionuclide that is 225-actinium (²²⁵Ac), 213-bismuth (²¹³Bi), 223-radium (²²³Ra), or 211-astatine (²¹¹At); or
a β-emitting radionuclide that is aluminium-[¹⁸F]fluoride ([¹⁸F]AlF), 68-gallium (68Ga), 90-yttrium (⁹⁰Y), 177-lutetium (¹⁷⁷Lu), 161-terbium (¹⁶¹Tb), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), 52-manganese (⁵²Mn), or 212-lead (²¹²Pb); or
a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

Embodiment 78. The compound of any one of embodiments 1-72, or a pharmaceutically acceptable salt thereof, wherein: the radionuclide of the radionuclide complex is In some embodiments, the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 99m-technetium (^{99m}Tc), 195m-platinum (^{195m}Pt), 225-actinium (²²⁵Ac), 213-bismuth (²¹³Bi), 223-radium (²²³Ra), 211-astatine (²¹¹At), aluminium-[¹⁸F]fluoride ([¹⁸F]AlF), 90-yttrium (⁹⁰Y), 175-lutetium (¹⁷⁵Lu), 177-lutetium (¹⁷⁷Lu), 161-terbium (¹⁶¹Tb), 186-rhenium (¹⁸⁶Re), 188-rhenium (¹⁸⁸Re), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), 153-samarium (¹⁵³Sm), 89-strontium (⁸⁹Sr), 198-gold (¹⁹⁸Au), 169-erbium (¹⁶⁹Er), 165-dysprosium (¹⁶⁵Dy), 99m-technetium (^{99m}Tc), 89-zirconium (⁸⁹Zr), 52-manganese (⁵²Mn), 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

Embodiment 79. The compound of any one of embodiments 1-72, or a pharmaceutically acceptable salt thereof, wherein: the radionuclide of the radionuclide complex is 64-copper (⁶⁴Cu), 67-copper (⁶⁷Cu), 90-yttrium (⁹⁰Y), 111-indium (¹¹¹In), 67-gallium (⁶⁷Ga), 68-gallium (⁶⁸Ga), 225-actinium (²²⁵Ac), 177-lutetium (¹⁷⁷Lu), 161-terbium (¹⁶¹Tb), or 212-lead (²¹²Pb).

Embodiment 80. A pharmaceutical composition comprising a compound of any one of embodiments 1-79, or a pharmaceutically acceptable salt thereof, and at least one pharmaceutically acceptable excipient.

Embodiment 81. The pharmaceutical composition of embodiment 80, wherein the pharmaceutical composition is formulated for administration to a mammal by intravenous administration.

Embodiment 82. A method for the treatment of cancer comprising administering to a mammal with cancer an effective amount of a compound of any one of embodiments 1-79, or a pharmaceutically acceptable salt thereof.

Embodiment 83. The method of embodiment 82, wherein the cancer comprises tumors and the tumors overexpress Protease-Activated Receptor-2 (PAR2).

Embodiment 84. The method of embodiment 82 or embodiment 83, wherein the cancer is colon cancer, pancreatic cancer, stomach cancer, esophageal cancer, rectal cancer, liver cancer, or lung cancer.

Embodiment 85. A method of killing tumors in a mammal that overexpress Protease-Activated Receptor-2 (PAR2) comprising administering to the mammal a compound of any one of embodiments 1-79, or a pharmaceutically acceptable salt thereof, wherein the compound of any one of embodiments 1-79, or a pharmaceutically acceptable salt thereof, comprises a therapeutic radionuclide.

Embodiment 86. The method of embodiment 85, wherein the mammal has been diagnosed with colon cancer, pancreatic cancer, stomach cancer, esophageal cancer, rectal cancer, liver cancer, or lung cancer.

Embodiment 87. A method for identifying tumors expressing Protease-Activated Receptor-2 (PAR2) in a mammal comprising administering to the mammal a compound of any one of embodiments 1-79, or a pharmaceutically acceptable salt thereof; and performing positron emission tomography (PET) analysis or single-photon emission computerized tomography (SPECT); wherein the compound of any one of embodiments 1-79, or a pharmaceutically acceptable salt thereof, comprises a diagnostic radionuclide.

Embodiment 88. A method for the in vivo imaging of tissues or organs in a mammal with tumors expressing Protease-Activated Receptor-2 (PAR2) comprising administering to the mammal a compound of any one of embodiments 1-79, or a pharmaceutically acceptable salt thereof; and performing positron emission tomography (PET) analysis or single-photon emission computerized tomography (SPECT); wherein the compound of any one of embodiments 1-79, or a pharmaceutically acceptable salt thereof, comprises a diagnostic radionuclide.

Embodiment 89. A compound of Formula (VII), or a pharmaceutically acceptable salt thereof:

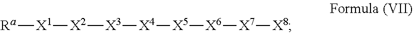

wherein:
R^{a} is —C(═O)—R¹⁰;
R¹⁰ is an aryl, heteroaryl, or partially unsaturated bicyclic heterocyclic ring, each optionally substituted with i to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹, and —R¹¹;
wherein any saturated carbon of R¹⁰ is optionally substituted with oxo (═O);
R¹¹ is an aryl or heteroaryl optionally substituted with I to 3 substituents that are each independently selected from the group consisting of —F, —Cl, —Br, —C₁-C₃ alkyl, —CF₃, —CN, —OCH₃, —NH—, and —NO₂;
X¹

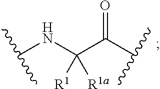

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

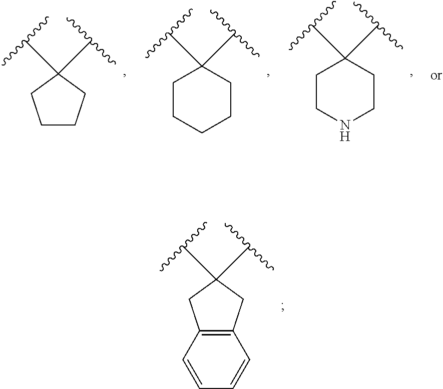 [SMILES: *C1(*)CCCC1 ; *C1(*)CCCCC1 ; *C1(*)CCNCC1 ; *C1(*)Cc2ccccc2C1]

X² is

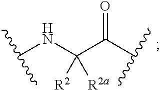

R² is -L²-R^{2b};
L² is absent, —CH₂—, or —CHPh-;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-l-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

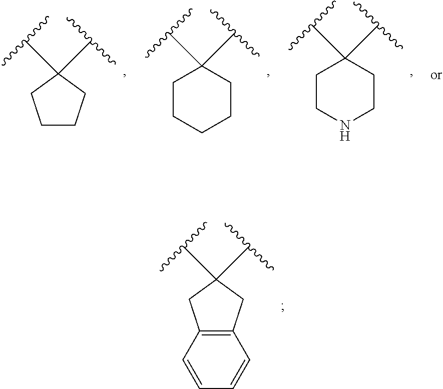 [SMILES: *C1(*)CCCC1 ; *C1(*)CCCCC1 ; *C1(*)CCNCC1 ; *C1(*)Cc2ccccc2C1]

X³ is glycine, alanine (Ala), trifluoromethylalanine, homoalanine, glutamic acid (Glu), glutamine (Gln), ornithine (Orn), lysine (Lys), serine (Ser), N-methylalanine (N-Me-Ala), 2-amino-2-methylpropanoic acid (AIB), 1-aminocyclopropane-1-carboxylic acid (ACC), norleucine (Nle), 3-aminoalanine, 2-amino-6-azidohexanoic acid, 2-amino-6-(4-hexyl-1H-1,2,3-triazol-1-yl)hexanoic acid, 2-amino-6-(4-((2-methoxyethoxy)methyl)-1H-1,2,3-triazol-1-yl)hexanoic acid, 2-amino-6-(4-(cyclopentylmethyl)-1H-1,2,3-triazol-1-yl)hexanoic acid, 2-amino-6-(4-benzyl-1H-1,2,3-triazol-l-yl)hexanoic acid, proline (Pro), cyclopropylglycine, or biphenylalanine (Bip);
X⁴ is arginine (Arg), 3-amino-6-guanidinohexanoic acid, N6-carbamimidoyl-lysine, 2-amino-5-ureidopentanoic acid, homoarginine, N-methyl-arginine, No-methyl-arginine, N^{ω}-nitro-arginine, (E)-N^{ω},N^{ω}′-dimethyl-arginine, (E)-N^{ω},N^{ω}′-diethyl-arginine, ornithine (Orn), lysine, phenylalanine (Phe), cyclohexylglycine (Chg), cyclohexylalanine (Cha), norleucine (Nle), 3-amino-alanine (H-Dap-OH), piperidinyl-1-alanine, NAO-dimethyl-asparagine (N⁴,N⁴-dimethyl-Asn), 2-(piperidin-4-yl)-glycine (Gly(piperidin-4-yl)), 3-pyridin-3-yl-beta-alanine (H-βAla(3-pyrid-3-yl)-OH), 2-(1-carbamimidoylpiperidin-4-yl)-glycine (Gly(1l-carbamimidoylpiperidin-4-yl)), 3-amino-3-(1-carbamimidoylpiperidin-3-yl)propanoic acid, phenylglycine (Phg), 4-fluorophenylalanine (Phe(4-F)), 4-aminophenylalanine (Phe(4-NH₂, 4-nitrophenylalanine (Phe(4-NO₂)), 4-carbamoylphenylalanine, 4-guanidinophenylalanine, asparagine (Asn), glutamic acid (Glu), proline, 4-ureidophenylalanine, (2-aminoethoxy)phenylalanine (Phe(4-OCH₂CH₂NH₂)), (pyridin-4-yl)alanine (3-Pal), (pyridin-4-yl)alanine (4-Pal), 3-(6-aminopyridin-3-yl)alanine ((3-Pal(6-NH₂)), 2-((2-aminopyridin-4-yl)-2-azaneyl)ethyl alanine, N6-nicotinoyl-Lys, 2-oxo-2-(piperazin-1-yl)ethyl alanine, 2-(piperidin-4-yl)ethylalanine, 3-((piperazin-1-yl)propyl)-glycine, 3-((4-methylpiperazin-1-yl)propyl)-glycine, 3-(4-ben ylpiperidin-1-yl)propylglycine, 3-((4-benzylpiperazin-1-yl)propyl)-glycine, N6-(1-benzylpiperidine-4-carbonyl)-Lys, 3-((3,4-dihydroisoquinolin-2(1H)-yl)propyl)-glycine, 2-pyridin-4-ylthynyl alanine, (4-(aminomethyl)-1H-1,2λ²,3λ²-triazol-1-yl)methyl)-glycine, (4-(piperazin-1-ylmethyl)-1H-1,2λ²,3λ²-triazol-1-yl)methyl)-glycine, 2-((1H-imidazol-4-yl)ethyl)glycine, or (4-((R)-2,6-dioxohexahydropyrimidine-4-carboxamido)phenyl)alanine;
or X⁴ is 4-methylpiperazine-2-carboxylic acid,
or X⁴ is

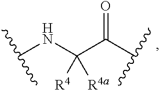

where R⁴ and R^{4a} are taken together with the carbon atom to which they are attached to form

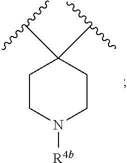

R^{4b} wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

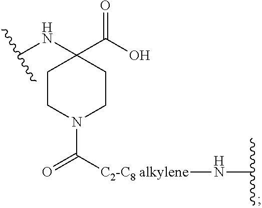

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-1)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

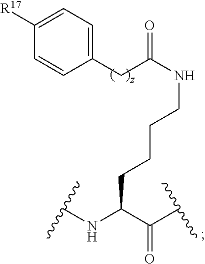

R¹⁷ is —CH₃ or —I;
z is 1, 2, or 3; and
X⁸ is absent, lysine (Lys), or glutamic acid (Glu);
L⁵, L⁶, and L⁷ are each independently —NR¹⁵-L³-L⁴-C(═O)— or substituted or unsubstituted piperazinylene-;
   L¹³ is absent, —C₁-C₆ alkylene, substituted or unsubstituted heterocycloalkylene, substituted or unsubstituted cyclohexylene, —(CH₂CH₂O)_{y}—, or —(CH₂CH₂NR¹⁶)_{y}—;
   L¹⁴ is absent, —C₁-C₆ alkylene, substituted or unsubstituted heterocycloalkylene, or substituted or unsubstituted cyclohexylene;
   R¹⁵ and R¹⁶ are each independently selected from H or —C₁-C₄ alkylene;
   y is 1, 2, 3, 4, 5, or 6; and
wherein the compound of Formula (I) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (VII) may be either —COOH or —CONH₂; and
wherein the compound of Formula (VII) is not:

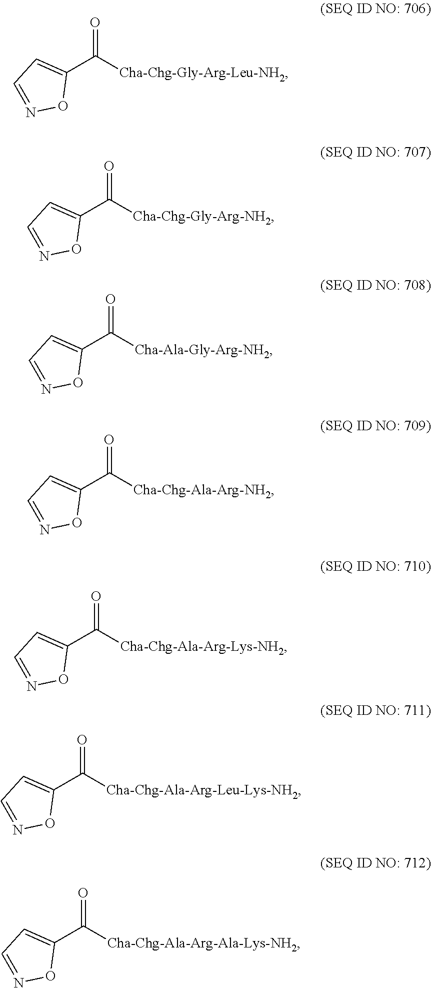

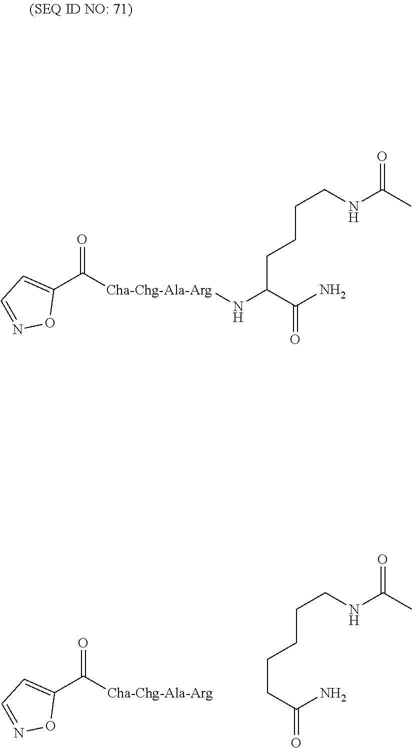

Embodiment 90. A compound of Formula (VII), or a pharmaceutically acceptable salt thereof:

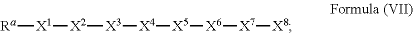

wherein:
R^{a} is —C(═O) R¹⁰;
R^{1O} is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R^{II}, or —O—C₁-C₆ alkylene-R¹, and —R¹¹; wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂:
X¹ is

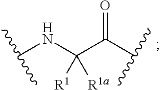

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

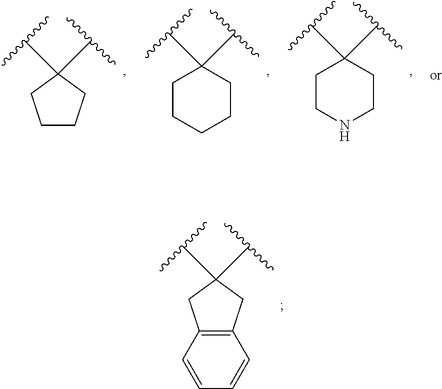 [SMILES: *C1(*)CCCC1 ; *C1(*)CCCCC1 ; *C1(*)CCNCC1 ; *C1(*)Cc2ccccc2C1]

X² is

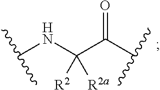

R² is -L²-R^{2b};
L² is absent, —CH₂—, or —CHPh-;
R^{2b} is pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

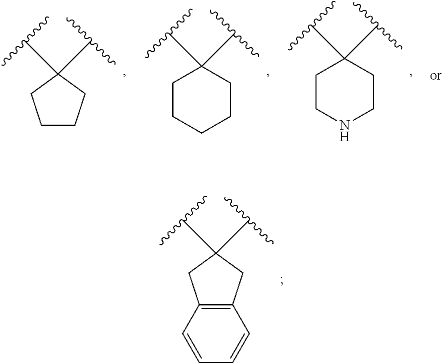 [SMILES: *C1(*)CCCC1 ; *C1(*)CCCCC1 ; *C1(*)CCNCC1 ; *C1(*)Cc2ccccc2C1]

X³ is alanine (Ala), trifluoromethylalanine, homoalanine, glutamic acid (Glu), glutamine (Gln), ornithine (Orn), lysine (Lys), serine (Ser), N-methylalanine (N-Me-Ala), 2-amino-2-methylpropanoic acid (AIB), 1-aminocyclopropane-1-carboxylic acid (ACC), norleucine (Nle), 3-aminoalanine, 2-amino-6-azidohexanoic acid, 2-amino-6-(4-hexyl-1H-1,2,3-triazol-1-yl)hexanoic acid, 2-amino-6-(4-((2-methoxyethoxy)methyl)-1H-1,2,3-triazol-1-yl)hexanoic acid, 2-amino-6-(4-(cyclopentylmethyl)-1H-1,2,3-triazol-1-yl)hexanoic acid, 2-amino-6-(4-benzyl-1H-1,2,3-triazol-1-yl)hexanoic acid, proline (Pro), cyclopropylglycine, or biphenylalanine (Bip);
X⁴ is arginine (Arg), 3-amino-6-guanidinohexanoic acid, N6-carbaamimidoyl-lysine, 2-amino-5-ureidopentanoic acid, homoarginine, N-methyl-arginine, NO-methyl-arginine, N^{ω}-nitro-arginine, (E)-N^{ω},N^{ω}′-dimethyl-arginine, (E)-N^{ω},N^{ω}′-diethyl-arginine, ornithine (Orn), lysine, phenylalanine (Phe), cyclohexylglycine (Chg), cyclohexylalanine (Cha), norleucine (Nle), 3-amino-alanine (H-Dap-OH), piperidinyl-1-alanine, N⁴,N⁴-dimethyl-asparagine (N⁴,N⁴-dimethyl-Asn), 2-(piperidin-4-yl)-glycine (Gly(piperidin-4-yl)), 3-pyridin-3-yl-beta-alanine (H-βAla(3-pyrid-3-yl)-OH), 2-(1-carbamimidoylpiperidin-4-yl)-glycine (Gly(1-carbamimidoylpiperdin-4-yl)), 3-amino-3-(1-carbamimidoylpiperidin-3-yl)propanoic acid, phenylglycine (Phg), 4-fluorophenylalanine (Phe(4-F)), 4-aminophenylalanine (Phe(4-NH₂)), 4-nitrophenylalanine (Phe(4-NO₂)), 4-carbamoylphenylalanine, 4-guanidinophenylalanine, asparagine (Asn), glutamic acid (Glu), proline, 4-ureidophenylalanine, (2-aminoethoxy)phenylalanine (Phe(4-OCH₂CH₂NH₂)), (pyridin-4-yl)alanine (3-Pal), (pyridin-4-yl)alanine (4-Pal), 3-(6-aminopyridin-3-yl)alanine ((3-Pal(6-NH₂)), 2-((2-aminopyridin-4-yl)-λ²-azaneyl)ethyl alanine, N6-nicotinoyl-Lys, 2-oxo-2-(piperazin-1-yl)ethyl alanine, 2-(piperidin-4-yl)ethylalanine, 3-((piperazin-1-yl)propyl)-glycine, 3-((4-methylpiperazin-1-yl)propyl)-glycine, 3-(4-benzylpiperidin-1-yl)propylglycine, 3-((4-benzylpiperazin-1-yl)propyl)-glycine, N6-(1-benzylpiperidine-4-carbonyl)-Lys, 3-((3,4-dihydroisoquinolin-2(1H)-yl)propyl)-glycine, 2-pyridin-4-ylthynyl alanine, (4-(aminomethyl)-1H-1,2λ²,3λ²-triazol-1-yl)methyl)-glycine, (4-(piperazin-1-ylmethyl)-1H-1,2λ²,3λ²-triazol-1-yl)methyl)-glycine, 2-((1H-imidazol-4-yl)ethyl)glycine, or (4-((R)-2,6-dioxohexahydropyrimidine-4-carboxamido)phenyl)alanine;
or X⁴ is 4-methylpiperazine-2-carboxylic acid,
or X⁴ is

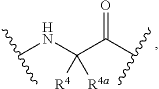

where R⁴ and R^{4a} are taken together with the carbon atom to which they are attached to form

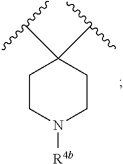

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

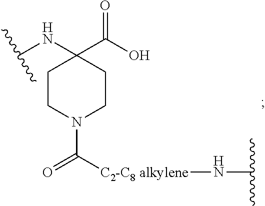

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-I)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

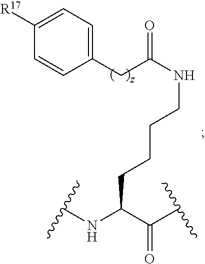

R¹⁷ is —CH₃ or —I;
z is 1, 2, or 3; and
X¹ is absent, lysine (Lys), or glutamic acid (Glu);
L⁵, L⁶, and L⁷ are each independently —NR¹⁵-L¹³-L¹⁴-C(═O)— or substituted or unsubstituted piperazinylene-;
   L¹³ is absent, —C₁-C₆ alkylene, substituted or unsubstituted heterocycloalkylene, substituted or unsubstituted cyclohexylene, —(CH₂CH₂O)_{y}—, or —(CH₂CH₂NR¹⁶)_{y}—;
   L¹⁴ is absent, —C₁-C₆ alkylene, substituted or unsubstituted heterocycloalkylene, substituted or unsubstituted cyclohexylene; and
   R¹⁵ and R¹⁶ are each independently selected from H or —C₁-C₄ alkylene;
   y is 1, 2, 3, 4, 5, or 6;
wherein the compound of Formula (VII) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (I) may be either —COOH or —CONH₂; and
wherein when X⁴ is Arginine, X⁵ is not absent, lysine (Lys), leucine (Leu), alanine (Ala), N6-acetyl-lysine, N6-(4-fluorobenzoyl)-Lys, 3-amino-alanine (H-Dap-OH), or (4-fluorobenzamido)methylglycine,

Embodiment 91. A compound of Formula (VII), or a pharmaceutically acceptable salt thereof:

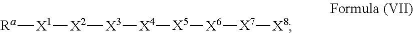

wherein:
R^{a} is —C(═O)—R¹⁰;
R¹⁰ is an aryl, heteroaryl, or partially unsaturated bicyclic heterocyclic ring, each optionally substituted with I to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, and —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
X¹

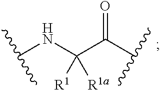

R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1-L-inden-2-yl, 3-cyclopropylphenyl, [1,1′-biphenyl]-4-yl, 4′-fluoro-[1,1′-biphenyl, 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form:

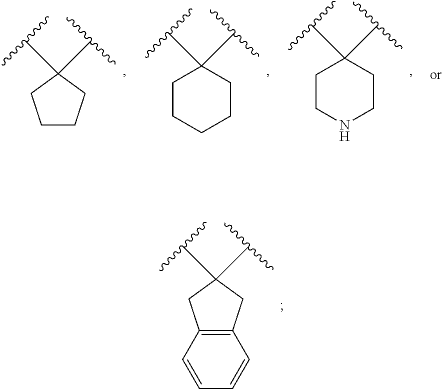 [SMILES: *C1(*)CCCC1 ; *C1(*)CCCCC1 ; *C1(*)CCNCC1 ; *C1(*)Cc2ccccc2C1]

X² is

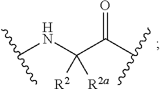

R² is -L²-R^{2b};
L² is absent, —CH₂—, or —CHPh-;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl, 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form:

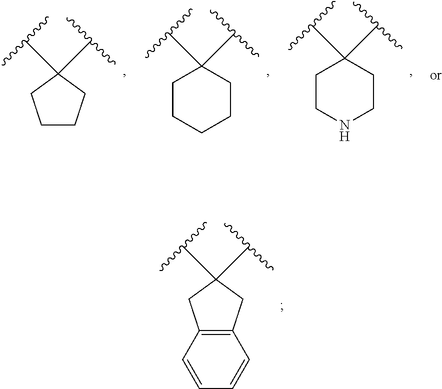 [SMILES: *C1(*)CCCC1 ; *C1(*)CCCCC1 ; *C1(*)CCNCC1 ; *C1(*)Cc2ccccc2C1]

X³ is glycine, alanine (Ala), trifluoromethylalanine, homoalanine, glutamic acid (Glu), glutamine (Gin), ornithine (Orn), lysine (Lys), serine (Ser), N-methylalanine (N-Me-Ala), 2-amino-2-methylpropanoic acid (AIB), 1-aminocyclopropane-1-carboxylic acid (ACC), norleucine (Nle), 3-aminoalanine, 2-amino-6-azidohexanoic acid, 2-amino-6-(4-hexyl-1H-1,2,3-triazol-1-yl)hexanoic acid, 2-amino-6-(4-((2-methoxyethoxy)methyl)-1H-1,2,3-triazol-1-yl)hexanoic acid, 2-amino-6-(4-(cyclopentylmethyl)-1H-1,2,3-triazol-1-yl)hexanoic acid, 2-amino-6-(4-benzyl-1H-1,2,3-triazol-1-yl)hexanoic acid, proline (Pro), cyclopropylglycine, or biphenylalanine (Bip);
X⁴ is 3-amino-6-guanidinohexanoic acid, N6-carbamimidoyl-lysine, 2-amino-5-ureidopentanoic acid, homoarginine, N-methyl-arginine, N^{ω}-methyl-arginine, N^{ω}-nitro-arginine, (E)-N^{ω},N^{ω}′-dimethyl-arginine, (E)-N^{ω},N^{ω}′-diethyl-arginine, ornithine (Om), lysine, phenylalanine (Phe), cyclohexylglycine (Chg), cyclohexylalanine (Cha), norleucine (Nle), 3-amino-alanine (H-Dap-OH), piperidinyl-1-alanine, N⁴,N⁴-dimethyl-asparagine ((N⁴,N⁴-dimethyl-Asn), 2-(piperidin-4-yl)-glycine (Gly(piperidin-4-yl)), 3-pyridin-3-yl-beta-alanine (H-βAla(3-pyrid-3-yl)-OH), 2-(1-carbamimidoylpiperidin-4-yl)-glycine (Gly(1-carbamimidoylpiperidin-4-yl)), 3-amino-3-(1-carbamimidoylpiperidin-3-yl)propanoic acid, phenylglycine (Phg), 4-fluorophenylalanine (Phe(4-F)), 4-aminophenylalanine (Phe(4-NH₂)), 4-nitrophenylalanine (Phe(4-NO₂)), 4-carbamoylphenylalanine, 4-guanidinophenylalanine, asparagine (Asn), glutamic acid (Glu), proline, 4-ureidophenylalanine, (2-aminoethoxy)phenylalanine (Phe(4-OCH₂CH₂NH₂)), (pyridin-4-yl)alanine (3-Pal), (pyridin-4-yl)alanine (4-Pal), 3-(6-aminopyridin-3-yl)alanine ((3-Pal(6-NH₂)), 2-((2-aminopyridin-4-yl)-λ²-azaneyl)ethyl alanine, N6-nicotinoyl-Lys, 2-oxo-2-(piperazin-1-yl)ethyl alanine, 2-(piperidin-4-yl)ethylalanine, 3-((piperazin-1-yl)propyl)-glycine, 3-((4-methylpiperazin-1-yl)propyl)-glycine, 3-(4-benzylpiperidin-1-yl)propylglycine, 3-((4-benzylpiperazin-1-yl)propyl)-glycine, N6-(1-benzylpiperidine-4-carbonyl)-Lys, 3-((3,4-dihydroisoquinolin-2(1H)-yl)propyl)-glycine, 2-pyridin-4-ylthynyl alanine, (4-(aminomethyl)-1H-1,2λ²,3λ²-triazol-1-yl)methyl)-glycine, (4-(piperazin-1-ylmethyl)-1H-1,2λ²,3λ²-triazol-1-yl)methyl)-glycine, 2-((1H-imidazol-4-yl)ethyl)glycine, or (4-((R)-2,6-dioxohexahydropyrimidine-4-carboxamido)phenyl)alanine;
or X⁴ is 4-methylpiperazine-2-carboxylic acid,
or X⁴ is

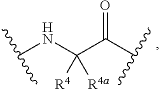

where R⁴ and R^{4a} are taken together with the carbon atom to which they are attached to form

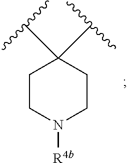

wherein R^{4′} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

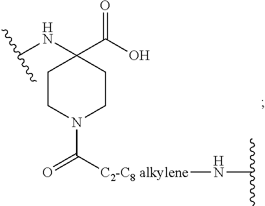

X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-1)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or

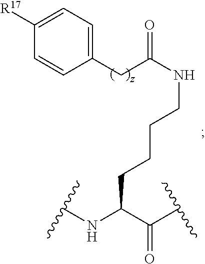

R¹⁷ is —CH₃ or —I;
z is 1, 2, or 3; and
X⁸ is absent, lysine (Lys), or glutamic acid (Glu);
L⁵, L⁶, and L⁷ are each independently —NR¹⁵-L¹³-L¹⁴-C(═O)— or substituted or unsubstituted piperazinylene-;
   L¹³ is absent, —C₁-C₆ alkylene, substituted or unsubstituted heterocycloalkylene, substituted or unsubstituted cyclohexylene, —(CH₂CH₂O)_{y}—, or —(CH₂CH₂NR⁶)_{y};
   L¹⁴ is absent, —C₁-C₆ alkylene, substituted or unsubstituted heterocycloalkylene, or substituted or unsubstituted cyclohexylene;
   R¹⁵ and R¹⁶ are each independently selected from H or —C₁-C₄ alkylene; and
y is 1, 2, 3, 4, 5, or 6;
wherein the compound of Formula (I) is optionally substituted with —C(═O)—C₁-C₁₄ alkylene-R¹², wherein R¹² is H, —CH₃, —COOH, —OK, —NH₂, or —NHCH₂CH₂N(CH₃)₃;
wherein any free —NI— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (I) may be either —COOH or —CONH₂.

Embodiment 92. The compound of any one of embodiments 89-91, wherein X² is

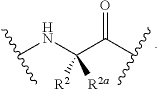

Embodiment 93. The compound of any one of embodiments 89-92, wherein X³ is glycine, (S)-alanine (Ala), (S)-trifluoromethylalanine, (S)-homoalanine, (S)-glutamic acid (Glu), (S)-glutamine (Gln), (S)-ornithin (Orn), (S)-lysine (Lys), (S)-serine (Ser), (S)—N-methylalanine (N-Me-Ala), 2-amino-2-methylpropanoic acid (AIB), 1-aminocyclopropane-1-carboxylic acid (ACC), (S)-norleucine (Nle), (S)-3-aminoalanine, (S)-2-amino-6-azidohexanoic acid, (S)-2-amino-6-(4-hexyl-1H-1,2,3-triazol-1-yl)hexanoic acid, (S)-2-amino-6-(4-((2-methoxyethoxy)methyl)-1H-1,2,3-triazol-1-yl)hexanoic acid, (S)-2-amino-6-(4-(cyclopentylmethyl)-1H-1,2,3-triazol-1-yl)hexanoic acid, (S)-2-amino-6-(4-benzyl-1H-1,2,3-triazol-1-yl)hexanoic acid, (S)-proline (Pro), (S)-cyclopropylglycine, or (S)-biphenylalanine (Bip).

Embodiment 94. The compound of any one of embodiments 89, 92, or 93, wherein X⁴ is (S)-arginine (Arg), (S)-3-amino-6-guanidinohexanoic acid, N6-carbamimidoyl-(S)-lysine, (S)-2-amino-5-ureidopentanoic acid, (S)-homoarginine, N-methyl-(S)-arginine, N^{ω}-methyl-(S)-arginine, N^{ω}-nitro-(S)-arginine, (E)-N^{ω},N^{ω}′-dimethyl-(S)-arginine, (E)-N^{ω},N^{ω}′-diethyl-(S)-arginine, (S)-ornithine (Orn), (S)-lysine, (S)-phenylalanine (Phe), (S)-cyclohexylglycine (Chg), (S)-cyclohexylalanine (Cha), norleucine (Nle), (S)-3-amino-alanine (H-Dap-OH), piperidinyl-1-(S)-alanine, N⁴,N⁴-dimethyl-(S)-asparagine (N⁴,N⁴-dimethyl-Asn), (S)-2-(piperidin-4-yl)-glycine (Gly(piperidin-4-yl)), (S)-3-pyridin-3-yl-beta-alanine (H-βAla(3-pyrid-3-yl)-OH), (S)-2-(1-carbamimidoylpiperidin-4-yl)-glycine (Gly(1-carbamimidoylpiperidin-4-yl)), (S)-3-amino-3-(1-carbamimidoylpiperidin-3-yl)propanoic acid, (S)-phenylglycine (Phg), (S)-4-fluorophenylalanine (Phe(4-F)), (S)-4-aminophenylalanine (Phe(4-NH₂)), (S)-4-nitrophenylalanine (Phe(4-NO₂)), (S)-4-carbamoylphenylalanine, (S)-4-guanidinophenylalanine, (S)-asparagine (Asn), (S)-glutamic acid (Glu), (S)-proline, (S)-4-ureidophenylalanine, (S)-(2-aminoethoxy)phenylalanine (Phe(4-OCH₂CH₂NH)), (S)-(pyridin-4-yl)alanine (3-Pal), (S)-(pyridin-4-yl)alanine (4-Pal), (S)-3-(6-aminopyridin-3-yl)alanine ((3-Pal(6-NH₂)), (S)-2-((2-aminopyridin-4-yl)-λ²-azaneyl)ethyl alanine, N6-nicotinoyl-Lys, 2-oxo-2-(piperazin-1-yl)ethyl alanine, (S)-2-(piperidin-4-yl)ethylalanine, (S)-3-((piperazin-1-yl)propyl)-glycine, (S)-3-((4-methylpiperazin-1-yl)propyl)-glycine, (S)-3-(4-benzylpiperidin-1-yl)propylglycine, (S)-3-((4-benzylpiperazin-1-yl)propyl)-glycine, N6-(1-benzylpiperidine-4-carbonyl)-Lys, (S)-3-((3,4-dihydroisoquinolin-2(1H)-yl)propyl)-glycine, (S)-2-pyridin-4-ylthynyl alanine; (S)-(4-(aminomethyl)-1H-1,2λ²,3λ²-triazol-1-yl)methyl)-glycine, (S)-(4-(piperazin-1-ylmethyl)-1H-1,2λ²,3λ²-triazol-1-yl)methyl)-glycine, (S)-2-((1H-imidazol-4-yl)ethyl)glycine, or (S)-(4-((R)-2,6-dioxohexahydropyrimidine-4-carboxamido)phenyl)alanine;
or X⁴ is (S)-4-methylpiperazine-2-carboxylic acid;
or X⁴ is

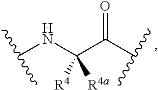

where R⁴ and R^{4a} are taken together with the carbon atom to which they are attached to form

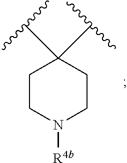

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

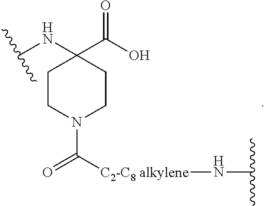

Embodiment 95. The compound of any one of embodiments 89, 92, or 93, wherein X⁴ is (S)-3-amino-6-guanidinohexanoic acid, N6-carbamimidoyl-(S)-lysine, (S)-2-amino-5-ureidopentanoic acid, (S)-homoarginine, N-methyl-(S)-arginine, N^{ω}-methyl-(S)-arginine, N^{ω}-nitro-(S)-arginine, (E)-N^{ω},N^{ω}′-dimenthyl-(S)-arginine, (E)-N^{ω},N^{ω}′-diethyl-(S)-arginine, (S)-ornithine (Orn), (S)-lysine, (S)-phenylalanine (Phe), (S)-cyclohexylglycine (Chg), (S)-cyclohexylalanine (Cha), norleucine (Nle), (S)-3-amino-alanine (H-Dap-OH), piperidinyl-1-(S)-alanine, N⁴,N⁴-dimethyl-(S)-asparagine (N⁴,N⁴-dimethyl-Asn), (S)-2-(piperidin-4-yl)-glycine (Gly(piperidin-4-yl)), (S)-3-pyridin-3-yl-beta-alanine (H-βAla(³-pyrid-3-yl)-OH), (S)-2-(1-carbamimidoylpiperidin-4-yl)-glycine (Gly(1-carbamimidoylpiperidin-4-yl)), (S)-3-amino-3-(1-carbamimidoylpiperidin-3-yl)propanoic acid, (S)-phenylglycine (Phg), (S)-4-fluorophenylalanine (Phe(4-F)), (S)-4-aminophenylalanine (Phe(4-NH₂)), (S)-4-nitrophenylalanine (Phe(4-NO₂)), (S)-4-carbamoylphenylalanine, (S)-4-guanidinophenylalanine, (S)-asparagine (Asn), (S)-glutamic acid (Glu), (S)-proline, (S)-4-ureidophenylalanine, (S)-(2-aminoethoxy)phenylalanine (Phe(4-OCH₂CH₂NH₂)), (S)-(pyridin-4-yl)alanine (3-Pal), (S)-(pyridin-4-yl)alanine (4-Pal), (S)-3-(6-aminopyridin-3-yl)alanine ((3-Pal(6-NH₂)), (S)-2-((2-aminopyridin-4-yl)-λ²-azaneyl)ethyl alanine, N6-nicotinoyl-Lys, 2-oxo-2-(piperazin-1-yl)ethyl alanine, (S)-2-(piperidin-4-yl)ethylalanine, (S)-3-((piperazin-1-yl)propyl)-glycine, (S)-3-((4-methylpiperazin-1-yl)propyl)-glycine, (S)-3-(4-benzylpiperidin-1-yl)propylglycine, (S)-3-((4-benzylpiperazin-1-yl)propyl)-glycine, N6-(I-benzylpiperidine-4-carbonyl)-Lys, (S)-3-((3,4-dihydroisoquinolin-2(1H)-yl)propyl)-glycine, (S)-2-pyridin-4-ylthynyl alanine, (S)-(4-(aminomethyl)-1H-1,2λ²,3λ²-triazol-1-yl)methyl)-glycine, (S)-(4-(piperazin-1-ylmethyl)-1H-1,2λ²,3λ²-triazol-1-yl)methyl)-glycine, (S)-2-((1H-imidazol-4-yl)ethyl)glycine, or (S)-(4-((R)-2,6-dioxohexahydropyrinidine-4-carboxamido)phenyl)alanine;
or X⁴ is (S)-4-methylpiperazine-2-carboxylic acid;
or X⁴ is

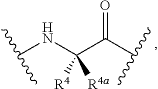

where R⁴ and R^{4a} are taken together with the carbon atom to which they are attached to form

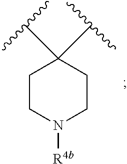

wherein R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
or X⁴ is

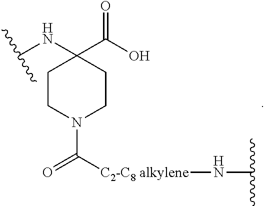

Embodiment 96. The compound of any one of embodiments 89, 92, or 93, or a pharmaceutically acceptable salt thereof, wherein
R^{a} is —C(═O)—R¹⁰;
   R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹, or —O—C₁-C₆ alkylene-R¹¹, and —R¹¹;
   wherein any saturated carbon of R¹⁰ is optionally substituted with oxo (═O);
   R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, and —NO₂;
X¹ is

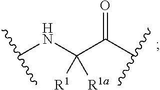

R¹ is -L¹-R^{1b};
   L is absent or —CH₂—;
   R^{1b} is cyclopentyl, cyclohexyl, cycloheptyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, 2-chlorophenyl, 3-chlorophenyl, 4-chlorophenyl, 2,4-dichlorophenyl, 3,4-dichlorophenyl, or 2,5-dichlorophenyl;
R^{1a} is hydrogen or methyl;
X² is

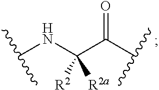

R² is -L²-R^{2b};
   L² is absent or —CH₂—;
   R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl, or 4′-methyl-[1,1′-biphenyl]-4-yl;
R^{2a} is hydrogen or methyl;
X³ is glycine, (S)-alanine (Ala), or norleucine (Nle);
X⁴ is (S)-arginine (Arg), (S)-ornithine (Orn), (S)-phenylalanine (Phe), (S)-2-(piperidin-4-yl)-glycine (Gly(piperidin-4-yl)), (S)-2-(1-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-(pyridin-4-yl)alanine (4-Pal), (S)-4-aminophenylalanine (Phe(4-NH₂)), (S)-3-(6-aminopyridin-3-yl)alanine ((3-Pal(6—NH₂)), or (S)-2-((2-aminopyridin-4-yl)-λ²-azaneyl)ethyl alanine;
X⁵ is alanine (Ala), lysine (Lys), ornithine (Orn),

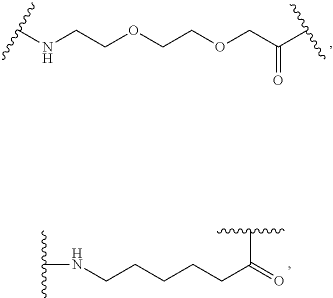 [SMILES: *NCCOCCOCC(*)=O ; *NCCCCCC(*)=O]

N6-(4-fluorobenzoyl)-Lys, β³-homolysine, 3-amino-alanine (H-Dap-OH), 4-nitrophenylalanine (Phe(4-NO₂)), or (4-fluorobenzamido)methylglycine;
X⁶ is absent, lysine (Lys), ornithine (Orn),

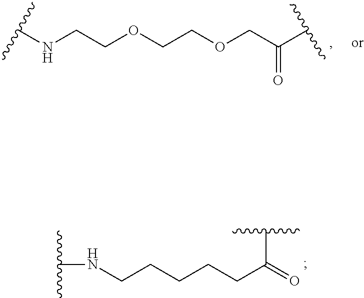 [SMILES: *NCCOCCOCC(*)=O ; *NCCCCCC(*)=O]

X⁷ is absent, lysine (Lys), ornithine (Orn), or

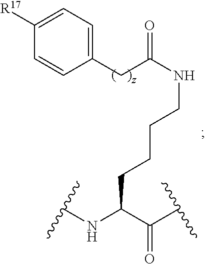

R¹⁷ is —CH₁₃ or —I;
z is 1, 2, or 3; and
X⁸ is absent;
wherein the compound of Formula (VII) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (VII) may be either —COOH or —CONH₂.

Embodiment 97. The compound of any one of embodiments 89-96, or a pharmaceutically acceptable salt thereof, wherein X¹ is

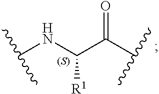

R¹ is -L¹-R^{1b};
L¹ is —CH₂—; and
R^{1b} is cyclopentyl, cyclohexyl, cycloheptyl, cyclooctyl, phenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, pyridin-4-yl, 2,3-dihydro-1H-inden-2-yl, 3-cyclopropylphenyl, or [1,1-biphenyl]-4-yl.

Embodiment 98. The compound of any one of embodiments 89-97, wherein R^{1b} is cyclohexyl or cycloheptyl.

Embodiment 99. The compound of any one of embodiments 89-97, wherein R^{1b} is phenyl, 4-fluorophenyl, 2,4-difluorophenyl, 3,4-difluorophenyl, 2,5-difluorophenyl, 3,4,5-trifluorophenyl, or perfluorophenyl.

Embodiment 100. The compound of any one of embodiments 89-99, wherein X² is

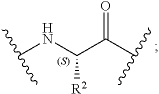

R² is -L²-R^{2b};
L² is absent; and
R^{2b} is cyclohexyl or cycloheptyl.

Embodiment 101. The compound of any one of embodiments 89-99, wherein X² is

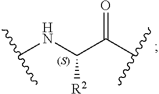

R² is -L²-R^{2b};
L² is absent; and
R^{2b} is 2,3-dihydro-1H-inden-2-yl or [1,1′-biphenyl]-4-yl.

Embodiment 102. The compound of any one of embodiments 89-101, wherein X³ is (S)-alanine (Ala) or (S)-norleucine (Nle).

Embodiment 103. The compound of any one of embodiments 89-101, or a pharmaceutically acceptable salt thereof, wherein X¹ is (S)-alanine (Ala).

Embodiment 104. The compound of any one of embodiments 89 or 92-103, or a pharmaceutically acceptable salt thereof, wherein X⁴ is (S)-arginine (Arg), (S)-ornithine (Orn), (S)-2-(piperidin-4-yl)-glycine (Gly(piperidin-4-yl)), (S)-2-(l-carbamimidoylpiperidin-4-yl)-glycine (Gly(1-carbamimidoylpiperidin-4-yl)), (S)-4-aminophenylalanine (Phe(4-NH₂)), (S)-(pyridin-3-yl)alanine (3-Pal), (S)-3-(6-aminopyridin-3-yl)alanine ((3-Pal(6-NH₂)), or (S)-2-((2-aminopyridin-4-yl)-λ²-azaneyl)ethyl alanine.

Embodiment 105. The compound of any one of embodiments 89 or 92-103, or a pharmaceutically acceptable salt thereof, wherein X⁴ is (S)-arginine (Arg).

Embodiment 106. The compound of any one of embodiments 89 or 92-103, or a pharmaceutically acceptable salt thereof, wherein X⁴ is (S)-2-(1-carbamimidoylpiperidin-4-yl)-glycine (Gly(1-carbamimidoylpiperidin-4-yl)).

Embodiment 107. The compound of any one of embodiments 89 or 92-103, or a pharmaceutically acceptable salt thereof, wherein X⁴ is (S)-2-amino-3-(4-aminophenyl)propanoic acid (Phe(4-NH₂)), (S)-2-amino-3-(6-aminopyridin-3-yl)propanoic acid, or (S)-2-((2-aminopyridin-4-yl)-λ²-azaneyl)ethyl alanine.

Embodiment 108. The compound of any one of embodiments 89 or 92-103, or a pharmaceutically acceptable salt thereof, wherein X⁴ is

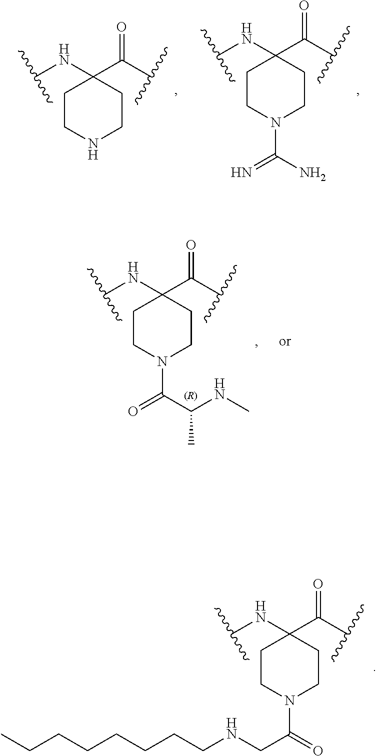

Embodiment 109. The compound of any one of embodiments 89 or 92-103, or a pharmaceutically acceptable salt thereof, wherein X⁴ is

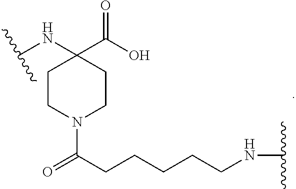 [SMILES: *NCCCCCC(=O)N1CCC(N*)(C(=O)O)CC1]

Embodiment 110. The compound of any one of embodiments 89 or 92-103, or a pharmaceutically acceptable salt thereof, wherein —X¹—X²—X³—X⁴— is:

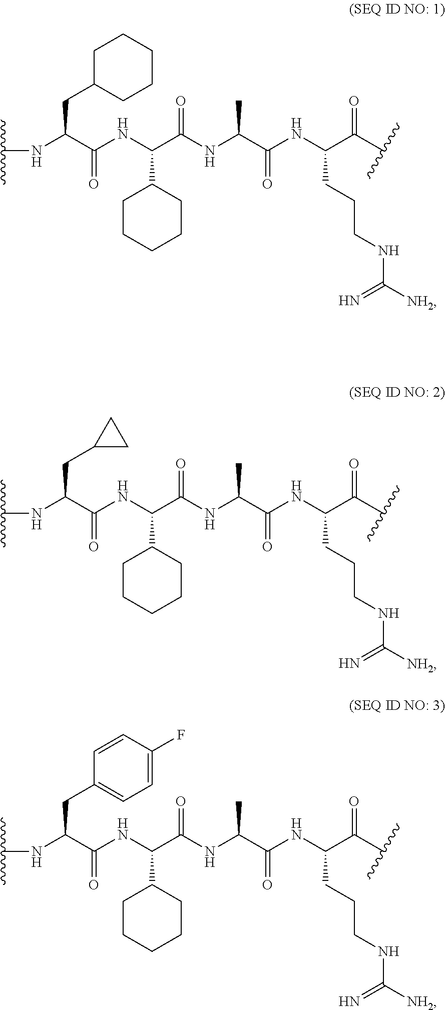 [SMILES: *N[C@@H](CC1CCCCC1)C(=O)N[C@H](C(=O)N[C@@H](C)C(=O)N[C@@H](CCCNC(=N)N)C(*)=O)C1CCCCC1 ; *N[C@@H](CC1CC1)C(=O)N[C@H](C(=O)N[C@@H](C)C(=O)N[C@@H](CCCNC(=N)N)C(*)=O)C1CCCCC1 ; *N[C@@H](Cc1ccc(F)cc1)C(=O)N[C@H](C(=O)N[C@@H](C)C(=O)N[C@@H](CCCNC(=N)N)C(*)=O)C1CCCCC1]

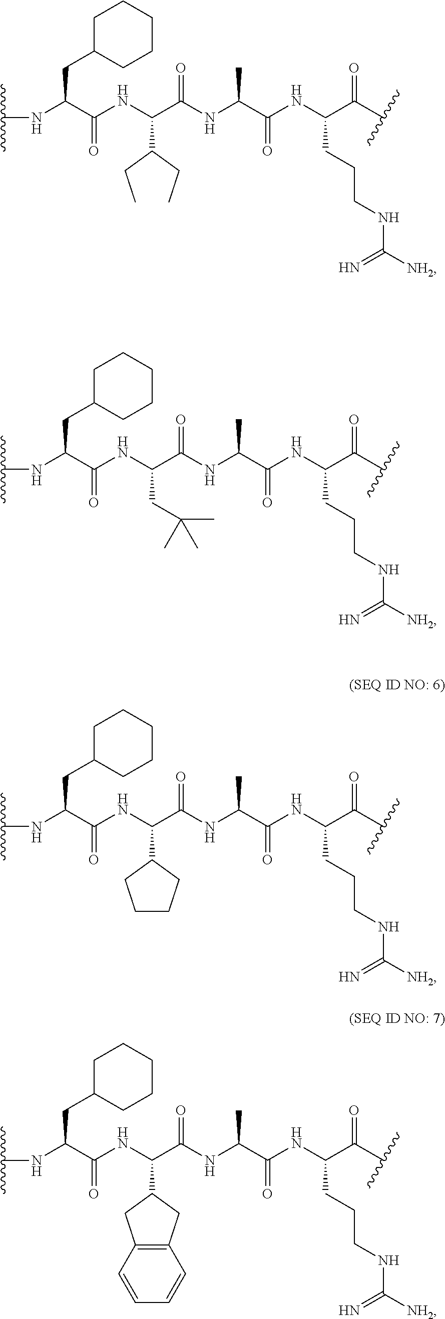 [SMILES: *N[C@@H](CC1CCCCC1)C(=O)N[C@H](C(=O)N[C@@H](C)C(=O)N[C@@H](CCCNC(=N)N)C(*)=O)C(CC)CC ; *N[C@@H](CC1CCCCC1)C(=O)N[C@@H](CC(C)(C)C)C(=O)N[C@@H](C)C(=O)N[C@@H](CCCNC(=N)N)C(*)=O ; *N[C@@H](CC1CCCCC1)C(=O)N[C@H](C(=O)N[C@@H](C)C(=O)N[C@@H](CCCNC(=N)N)C(*)=O)C1CCCC1 ; *N[C@@H](CC1CCCCC1)C(=O)N[C@H](C(=O)N[C@@H](C)C(=O)N[C@@H](CCCNC(=N)N)C(*)=O)C1Cc2ccccc2C1]

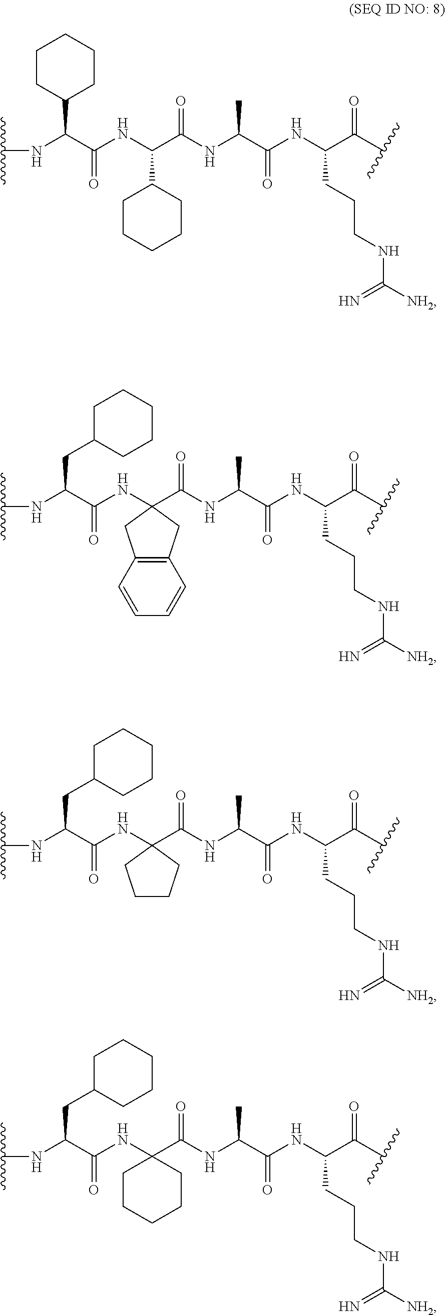 [SMILES: *N[C@H](C(=O)N[C@H](C(=O)N[C@@H](C)C(=O)N[C@@H](CCCNC(=N)N)C(*)=O)C1CCCCC1)C1CCCCC1 ; *N[C@@H](CC1CCCCC1)C(=O)NC1(C(=O)N[C@@H](C)C(=O)N[C@@H](CCCNC(=N)N)C(*)=O)Cc2ccccc2C1 ; *N[C@@H](CC1CCCCC1)C(=O)NC1(C(=O)N[C@@H](C)C(=O)N[C@@H](CCCNC(=N)N)C(*)=O)CCCC1 ; *N[C@@H](CC1CCCCC1)C(=O)NC1(C(=O)N[C@@H](C)C(=O)N[C@@H](CCCNC(=N)N)C(*)=O)CCCCC1]

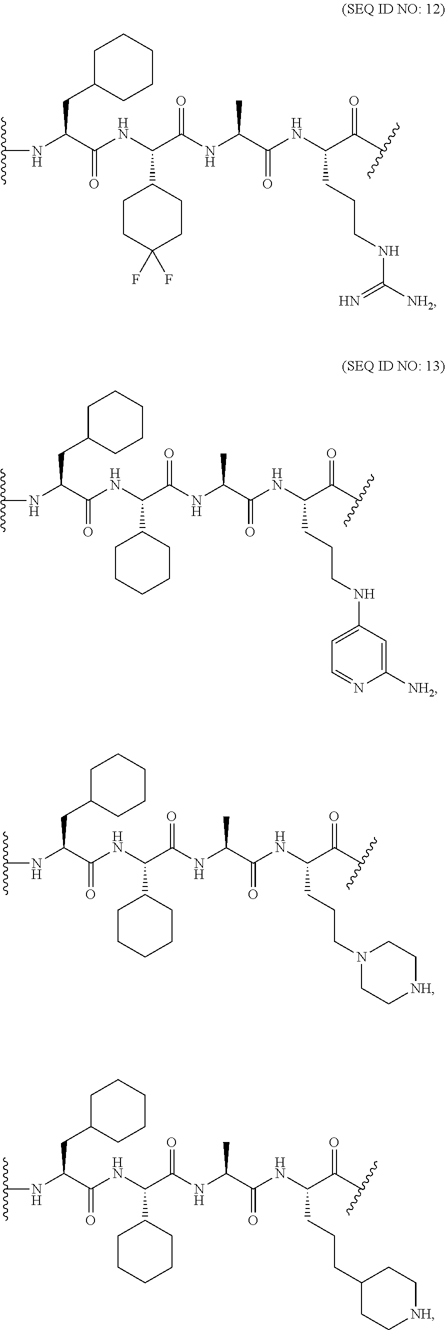 [SMILES: *N[C@@H](CC1CCCCC1)C(=O)N[C@H](C(=O)N[C@@H](C)C(=O)N[C@@H](CCCNC(=N)N)C(*)=O)C1CCC(F)(F)CC1 ; *N[C@@H](CC1CCCCC1)C(=O)N[C@H](C(=O)N[C@@H](C)C(=O)N[C@@H](CCCNc1ccnc(N)c1)C(*)=O)C1CCCCC1 ; *N[C@@H](CC1CCCCC1)C(=O)N[C@H](C(=O)N[C@@H](C)C(=O)N[C@@H](CCCN1CCNCC1)C(*)=O)C1CCCCC1 ; *N[C@@H](CC1CCCCC1)C(=O)N[C@H](C(=O)N[C@@H](C)C(=O)N[C@@H](CCCC1CCNCC1)C(*)=O)C1CCCCC1]

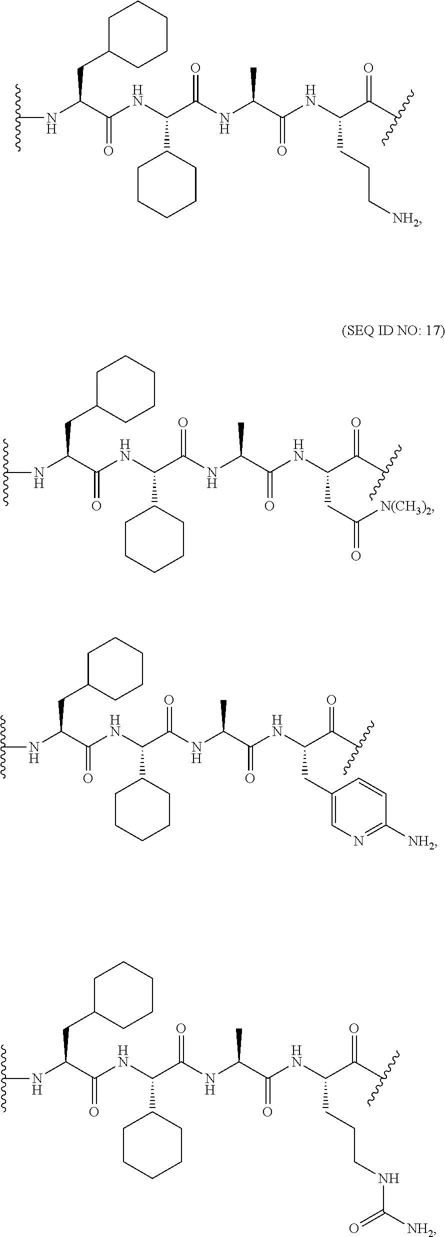 [SMILES: *N[C@@H](CC1CCCCC1)C(=O)N[C@H](C(=O)N[C@@H](C)C(=O)N[C@@H](CCCN)C(*)=O)C1CCCCC1 ; *N[C@@H](CC1CCCCC1)C(=O)N[C@H](C(=O)N[C@@H](C)C(=O)N[C@@H](CC(=O)N(C)C)C(*)=O)C1CCCCC1 ; *N[C@@H](CC1CCCCC1)C(=O)N[C@H](C(=O)N[C@@H](C)C(=O)N[C@@H](Cc1ccc(N)nc1)C(*)=O)C1CCCCC1 ; *N[C@@H](CC1CCCCC1)C(=O)N[C@H](C(=O)N[C@@H](C)C(=O)N[C@@H](CCCNC(N)=O)C(*)=O)C1CCCCC1]

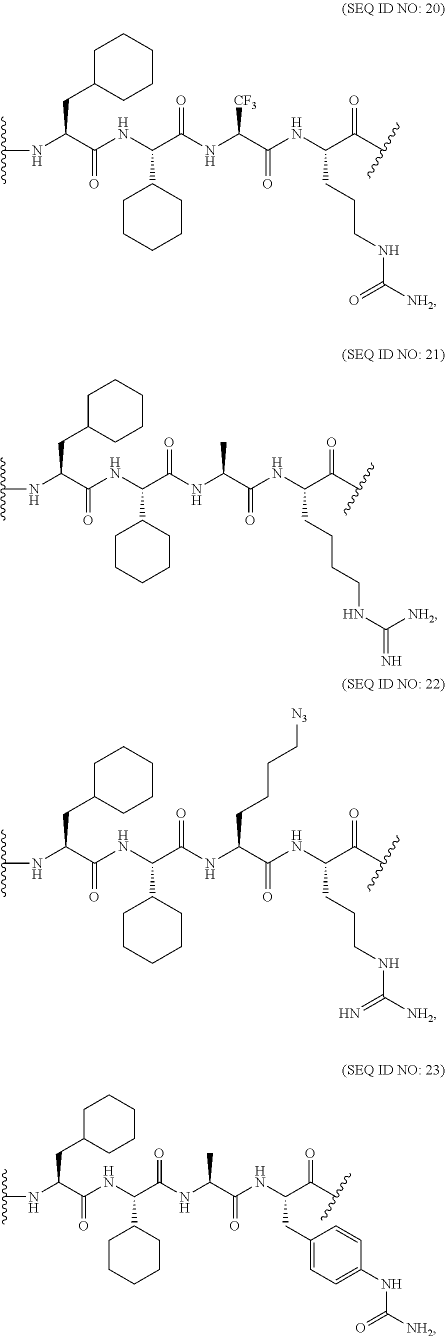 [SMILES: *N[C@@H](CC1CCCCC1)C(=O)N[C@H](C(=O)N[C@H](C(=O)N[C@@H](CCCNC(N)=O)C(*)=O)C(F)(F)F)C1CCCCC1 ; *N[C@@H](CC1CCCCC1)C(=O)N[C@H](C(=O)N[C@@H](C)C(=O)N[C@@H](CCCCNC(=N)N)C(*)=O)C1CCCCC1 ; *N[C@@H](CC1CCCCC1)C(=O)N[C@H](C(=O)N[C@@H](CCCCN=[N+]=[N-])C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N[C@@H](Cc1ccc(NC(N)=O)cc1)C(*)=O)C1CCCCC1]

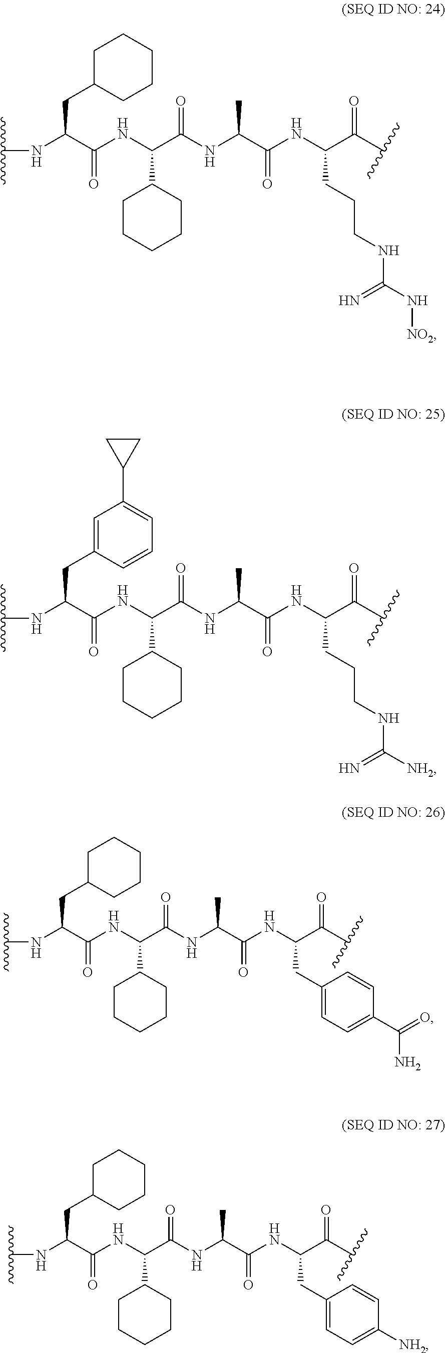 [SMILES: *N[C@@H](CC1CCCCC1)C(=O)N[C@H](C(=O)N[C@@H](C)C(=O)N[C@@H](CCCNC(=N)N[N+](=O)[O-])C(*)=O)C1CCCCC1 ; *N[C@@H](Cc1cccc(C2CC2)c1)C(=O)N[C@H](C(=O)N[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N[C@@H](Cc1ccc(C(N)=O)cc1)C(*)=O)C1CCCCC1 ; *N[C@@H](CC1CCCCC1)C(=O)N[C@H](C(=O)N[C@@H](C)C(=O)N[C@@H](Cc1ccc(N)cc1)C(*)=O)C1CCCCC1]

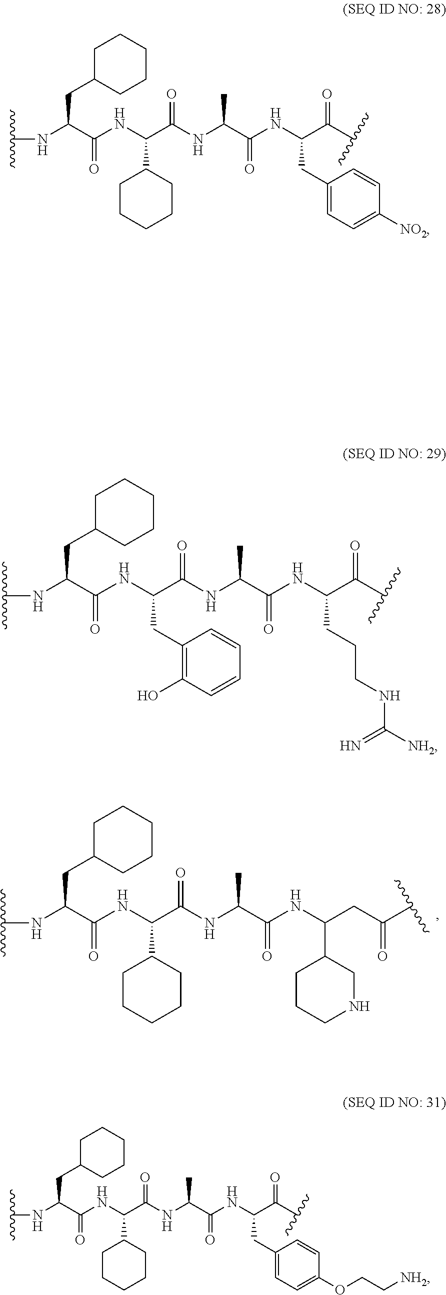 [SMILES: *N[C@@H](CC1CCCCC1)C(=O)N[C@H](C(=O)N[C@@H](C)C(=O)N[C@@H](Cc1ccc([N+](=O)[O-])cc1)C(*)=O)C1CCCCC1 ; *N[C@@H](CC1CCCCC1)C(=O)N[C@@H](Cc1ccccc1O)C(=O)N[C@@H](C)C(=O)N[C@@H](CCCNC(=N)N)C(*)=O ; *N[C@@H](CC1CCCCC1)C(=O)N[C@H](C(=O)N[C@@H](C)C(=O)NC(CC(*)=O)C1CCCNC1)C1CCCCC1 ; *N[C@@H](CC1CCCCC1)C(=O)N[C@H](C(=O)N[C@@H](C)C(=O)N[C@@H](Cc1ccc(OCCN)cc1)C(*)=O)C1CCCCC1]

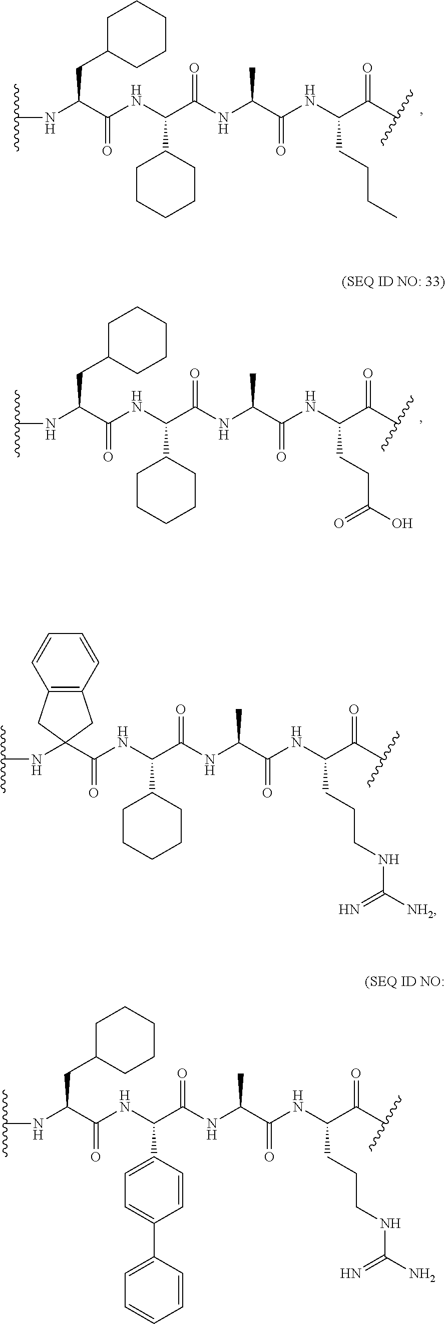 [SMILES: *N[C@@H](CC1CCCCC1)C(=O)N[C@H](C(=O)N[C@@H](C)C(=O)N[C@@H](CCCC)C(*)=O)C1CCCCC1 ; *N[C@@H](CC1CCCCC1)C(=O)N[C@H](C(=O)N[C@@H](C)C(=O)N[C@@H](CCC(=O)O)C(*)=O)C1CCCCC1 ; *NC1(C(=O)N[C@H](C(=O)N[C@@H](C)C(=O)N[C@@H](CCCNC(=N)N)C(*)=O)C2CCCCC2)Cc2ccccc2C1 ; *N[C@@H](CC1CCCCC1)C(=O)N[C@H](C(=O)N[C@@H](C)C(=O)N[C@@H](CCCNC(=N)N)C(*)=O)c1ccc(-c2ccccc2)cc1]

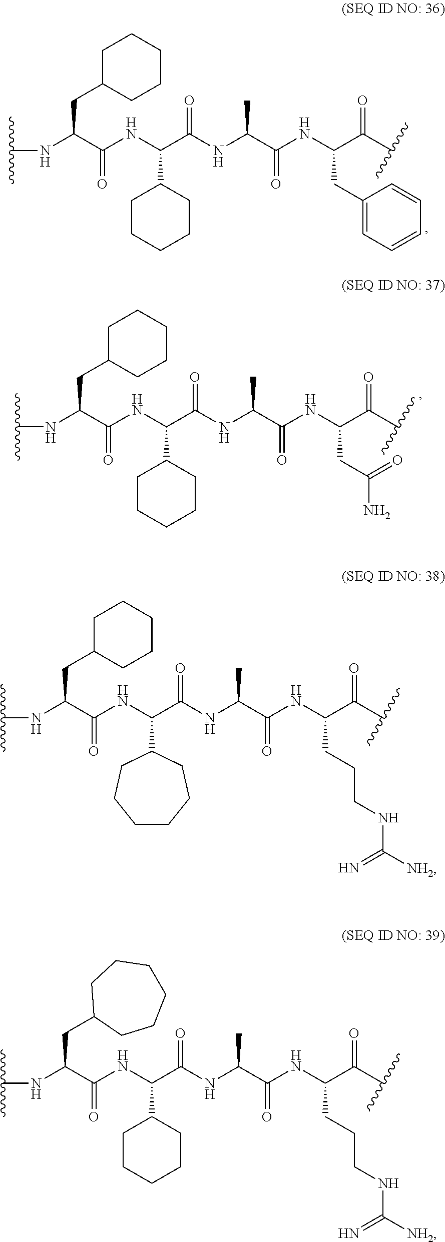 [SMILES: *N[C@@H](CC1CCCCC1)C(=O)N[C@H](C(=O)N[C@@H](C)C(=O)N[C@@H](Cc1ccccc1)C(*)=O)C1CCCCC1 ; *N[C@@H](CC1CCCCC1)C(=O)N[C@H](C(=O)N[C@@H](C)C(=O)N[C@@H](CC(N)=O)C(*)=O)C1CCCCC1 ; *N[C@@H](CC1CCCCC1)C(=O)N[C@H](C(=O)N[C@@H](C)C(=O)N[C@@H](CCCNC(=N)N)C(*)=O)C1CCCCCC1 ; *N[C@@H](CC1CCCCCC1)C(=O)N[C@H](C(=O)N[C@@H](C)C(=O)N[C@@H](CCCNC(=N)N)C(*)=O)C1CCCCC1]

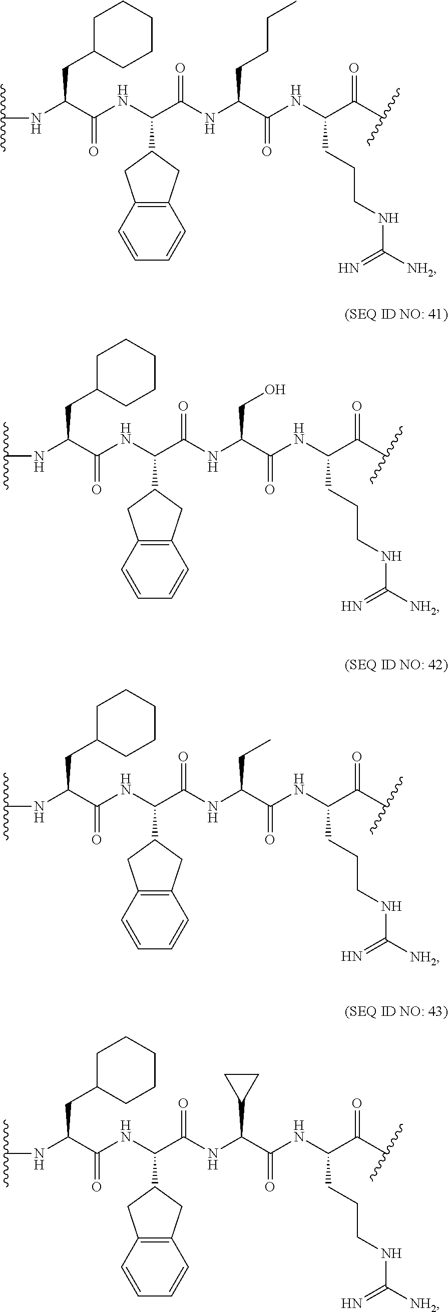 [SMILES: *N[C@@H](CC1CCCCC1)C(=O)N[C@H](C(=O)N[C@@H](CCCC)C(=O)N[C@@H](CCCNC(=N)N)C(*)=O)C1Cc2ccccc2C1 ; *N[C@@H](CC1CCCCC1)C(=O)N[C@H](C(=O)N[C@@H](CO)C(=O)N[C@@H](CCCNC(=N)N)C(*)=O)C1Cc2ccccc2C1 ; *N[C@@H](CC1CCCCC1)C(=O)N[C@H](C(=O)N[C@@H](CC)C(=O)N[C@@H](CCCNC(=N)N)C(*)=O)C1Cc2ccccc2C1 ; *N[C@@H](CC1CCCCC1)C(=O)N[C@H](C(=O)N[C@H](C(=O)N[C@@H](CCCNC(=N)N)C(*)=O)C1CC1)C1Cc2ccccc2C1]

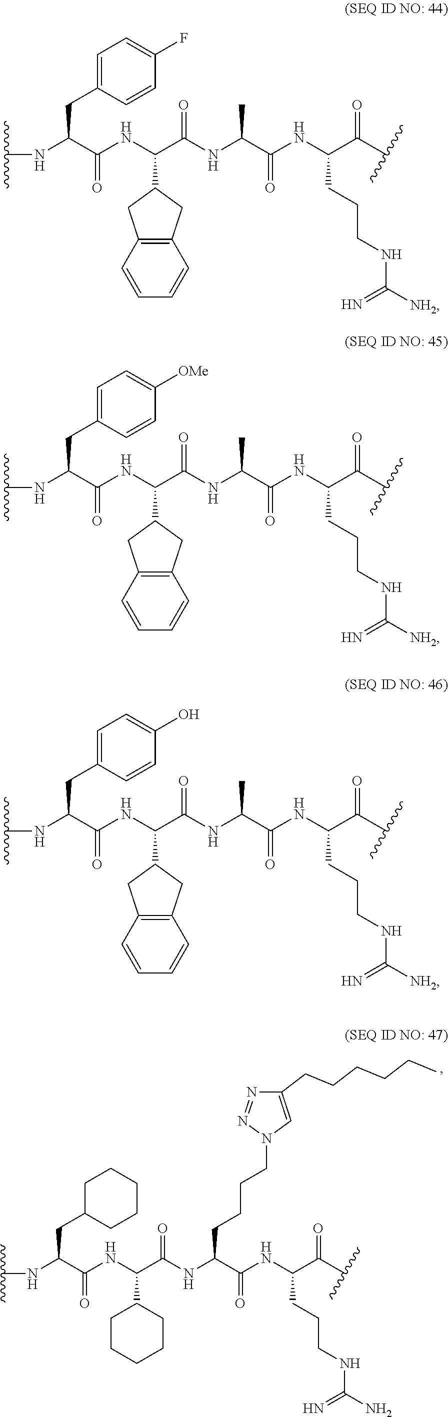 [SMILES: *N[C@@H](Cc1ccc(F)cc1)C(=O)N[C@H](C(=O)N[C@@H](C)C(=O)N[C@@H](CCCNC(=N)N)C(*)=O)C1Cc2ccccc2C1 ; *N[C@@H](Cc1ccc(OC)cc1)C(=O)N[C@H](C(=O)N[C@@H](C)C(=O)N[C@@H](CCCNC(=N)N)C(*)=O)C1Cc2ccccc2C1 ; *N[C@@H](Cc1ccc(O)cc1)C(=O)N[C@H](C(=O)N[C@@H](C)C(=O)N[C@@H](CCCNC(=N)N)C(*)=O)C1Cc2ccccc2C1 ; *N[C@@H](CC1CCCCC1)C(=O)N[C@H](C(=O)N[C@@H](CCCCn1cc(CCCCCC)nn1)C(=O)N[C@@H](CCCNC(=N)N)C(*)=O)C1CCCCC1]

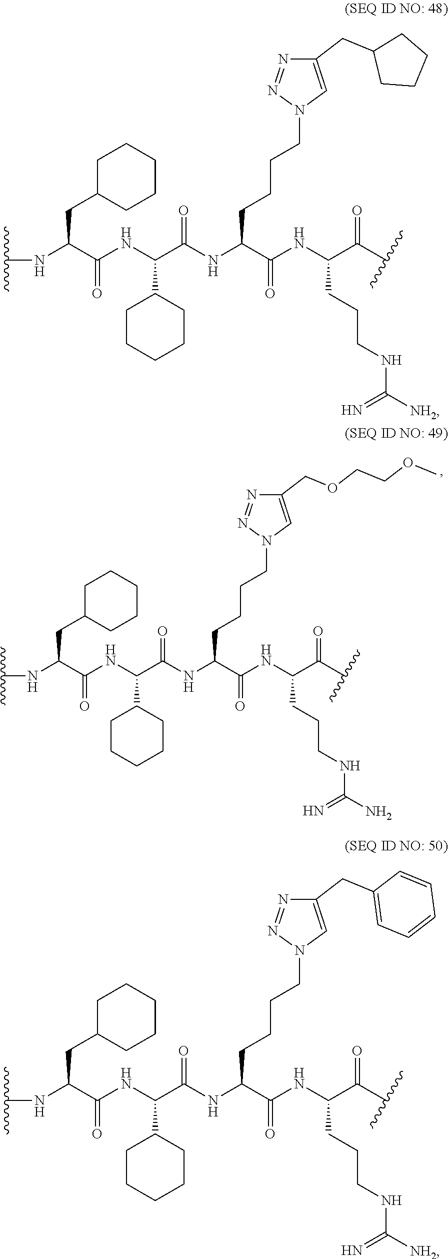 [SMILES: *N[C@@H](CC1CCCCC1)C(=O)N[C@H](C(=O)N[C@@H](CCCCn1cc(CC2CCCC2)nn1)C(=O)N[C@@H](CCCNC(=N)N)C(*)=O)C1CCCCC1 ; *N[C@@H](CC1CCCCC1)C(=O)N[C@H](C(=O)N[C@@H](CCCCn1cc(COCCOC)nn1)C(=O)N[C@@H](CCCNC(=N)N)C(*)=O)C1CCCCC1 ; *N[C@@H](CC1CCCCC1)C(=O)N[C@H](C(=O)N[C@@H](CCCCn1cc(Cc2ccccc2)nn1)C(=O)N[C@@H](CCCNC(=N)N)C(*)=O)C1CCCCC1]

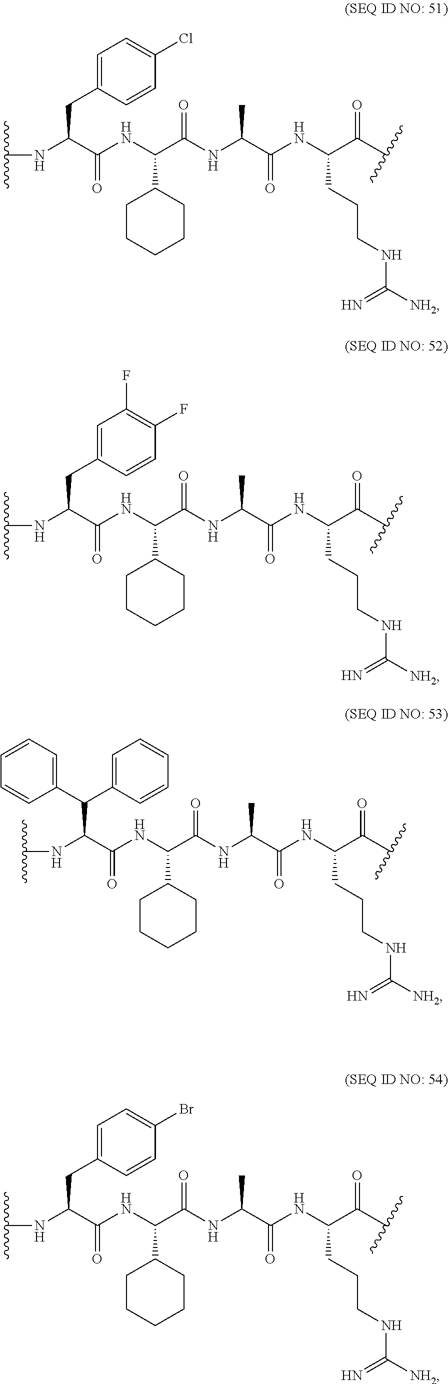 [SMILES: *N[C@@H](Cc1ccc(Cl)cc1)C(=O)N[C@H](C(=O)N[C@@H](C)C(=O)N[C@@H](CCCNC(=N)N)C(*)=O)C1CCCCC1 ; *N[C@@H](Cc1ccc(F)c(F)c1)C(=O)N[C@H](C(=O)N[C@@H](C)C(=O)N[C@@H](CCCNC(=N)N)C(*)=O)C1CCCCC1 ; *N[C@H](C(=O)N[C@H](C(=O)N[C@@H](C)C(=O)N[C@@H](CCCNC(=N)N)C(*)=O)C1CCCCC1)C(c1ccccc1)c1ccccc1 ; *N[C@@H](Cc1ccc(Br)cc1)C(=O)N[C@H](C(=O)N[C@@H](C)C(=O)N[C@@H](CCCNC(=N)N)C(*)=O)C1CCCCC1]

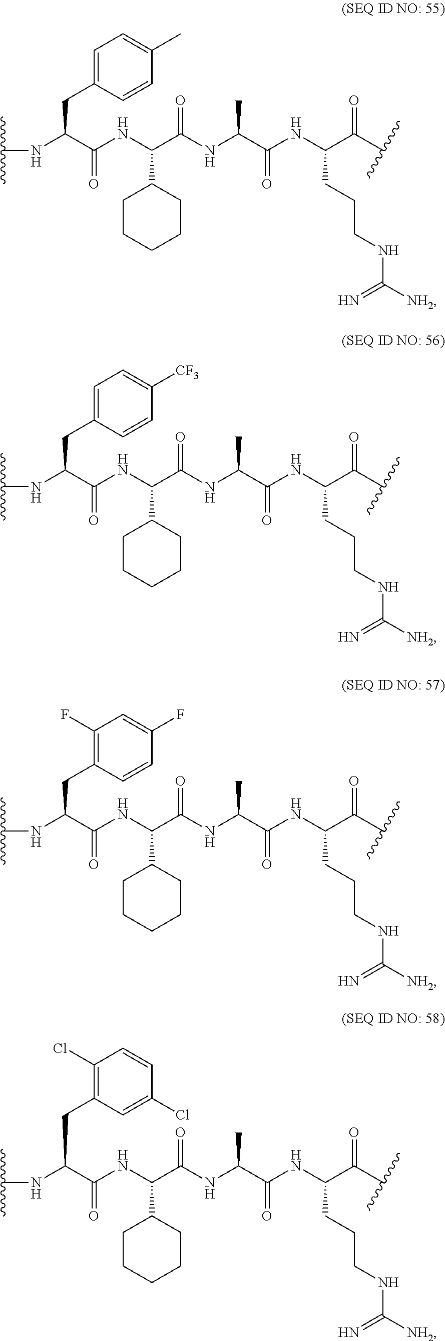 [SMILES: *N[C@@H](Cc1ccc(C)cc1)C(=O)N[C@H](C(=O)N[C@@H](C)C(=O)N[C@@H](CCCNC(=N)N)C(*)=O)C1CCCCC1 ; *N[C@@H](Cc1ccc(C(F)(F)F)cc1)C(=O)N[C@H](C(=O)N[C@@H](C)C(=O)N[C@@H](CCCNC(=N)N)C(*)=O)C1CCCCC1 ; *N[C@@H](Cc1ccc(F)cc1F)C(=O)N[C@H](C(=O)N[C@@H](C)C(=O)N[C@@H](CCCNC(=N)N)C(*)=O)C1CCCCC1 ; *N[C@@H](Cc1cc(Cl)ccc1Cl)C(=O)N[C@H](C(=O)N[C@@H](C)C(=O)N[C@@H](CCCNC(=N)N)C(*)=O)C1CCCCC1]

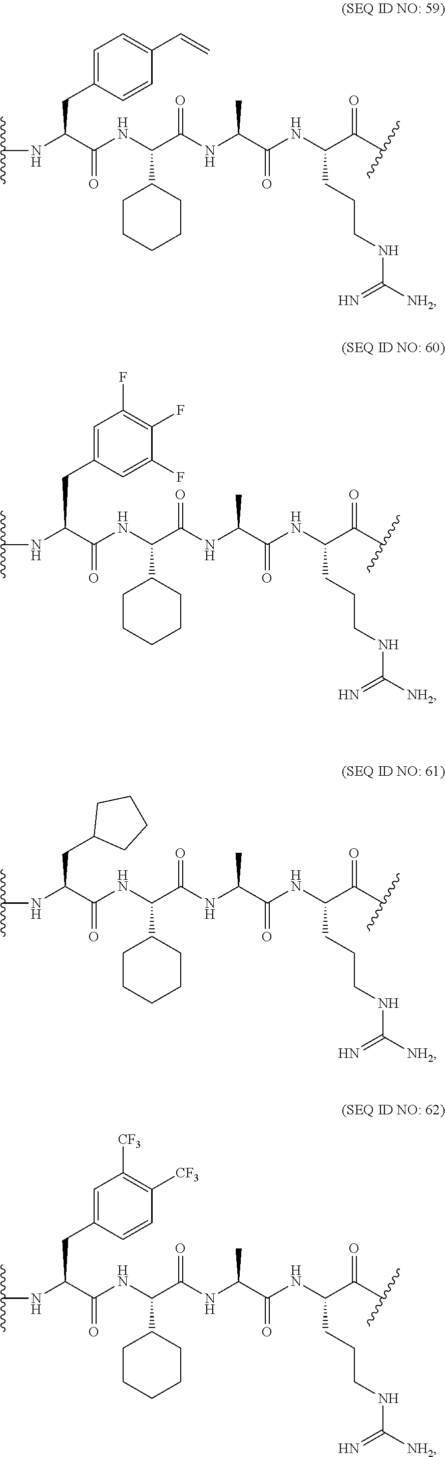 [SMILES: *N[C@@H](Cc1ccc(C=C)cc1)C(=O)N[C@H](C(=O)N[C@@H](C)C(=O)N[C@@H](CCCNC(=N)N)C(*)=O)C1CCCCC1 ; *N[C@@H](Cc1cc(F)c(F)c(F)c1)C(=O)N[C@H](C(=O)N[C@@H](C)C(=O)N[C@@H](CCCNC(=N)N)C(*)=O)C1CCCCC1 ; *N[C@@H](CC1CCCC1)C(=O)N[C@H](C(=O)N[C@@H](C)C(=O)N[C@@H](CCCNC(=N)N)C(*)=O)C1CCCCC1 ; *N[C@@H](Cc1ccc(C(F)(F)F)c(C(F)(F)F)c1)C(=O)N[C@H](C(=O)N[C@@H](C)C(=O)N[C@@H](CCCNC(=N)N)C(*)=O)C1CCCCC1]

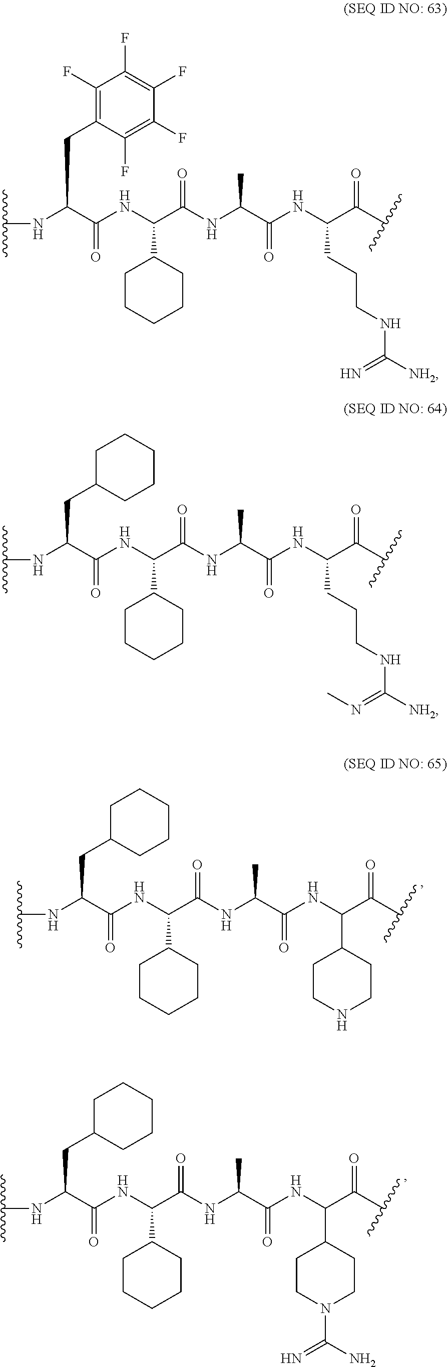 [SMILES: *N[C@@H](Cc1c(F)c(F)c(F)c(F)c1F)C(=O)N[C@H](C(=O)N[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](C)C(=O)N[C@@H](CCCN/C(N)=N\C)C(*)=O)C1CCCCC1 ; *N[C@@H](CC1CCCCC1)C(=O)N[C@H](C(=O)N[C@@H](C)C(=O)NC(C(*)=O)C1CCNCC1)C1CCCCC1 ; *N[C@@H](CC1CCCCC1)C(=O)N[C@H](C(=O)N[C@@H](C)C(=O)NC(C(*)=O)C1CCN(C(=N)N)CC1)C1CCCCC1]

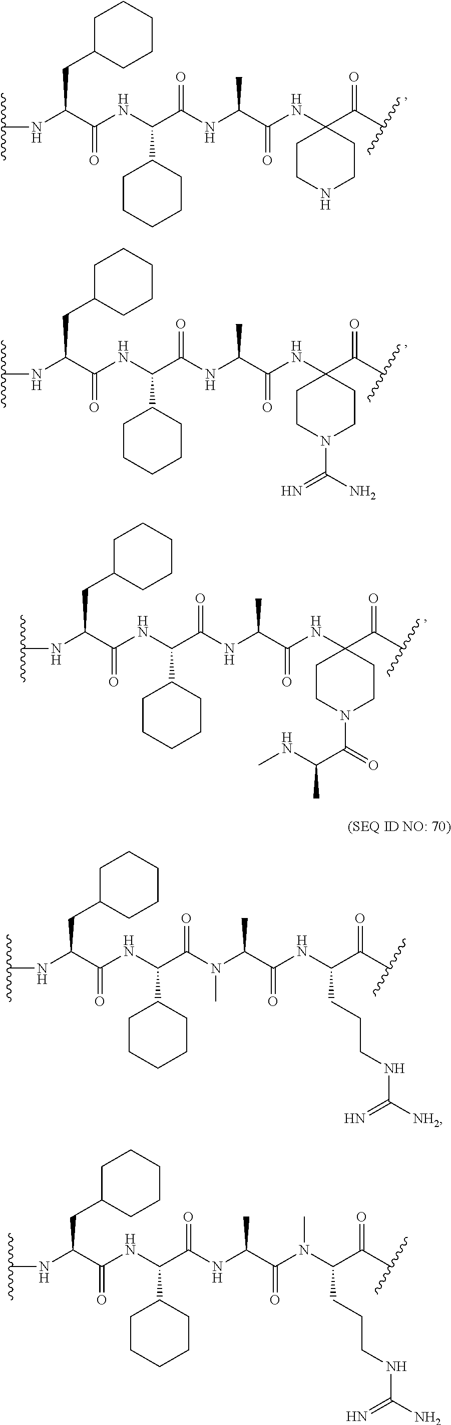

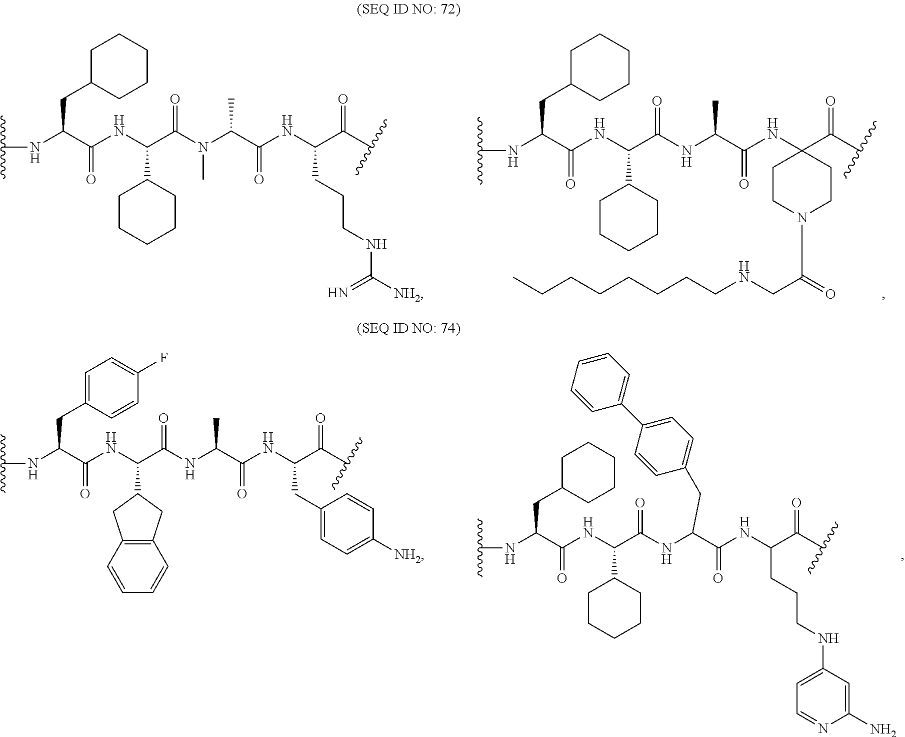 [SMILES: *N[C@@H](CC1CCCCC1)C(=O)N[C@H](C(=O)N(C)[C@H](C)C(=O)N[C@@H](CCCNC(=N)N)C(*)=O)C1CCCCC1 ; *N[C@@H](CC1CCCCC1)C(=O)N[C@H](C(=O)N[C@@H](C)C(=O)NC1(C(*)=O)CCN(C(=O)CNCCCCCCCC)CC1)C1CCCCC1 ; *N[C@@H](Cc1ccc(F)cc1)C(=O)N[C@H](C(=O)N[C@@H](C)C(=O)N[C@@H](Cc1ccc(N)cc1)C(*)=O)C1Cc2ccccc2C1 ; *N[C@@H](CC1CCCCC1)C(=O)N[C@H](C(=O)NC(Cc1ccc(-c2ccccc2)cc1)C(=O)NC(CCCNc1ccnc(N)c1)C(*)=O)C1CCCCC1]

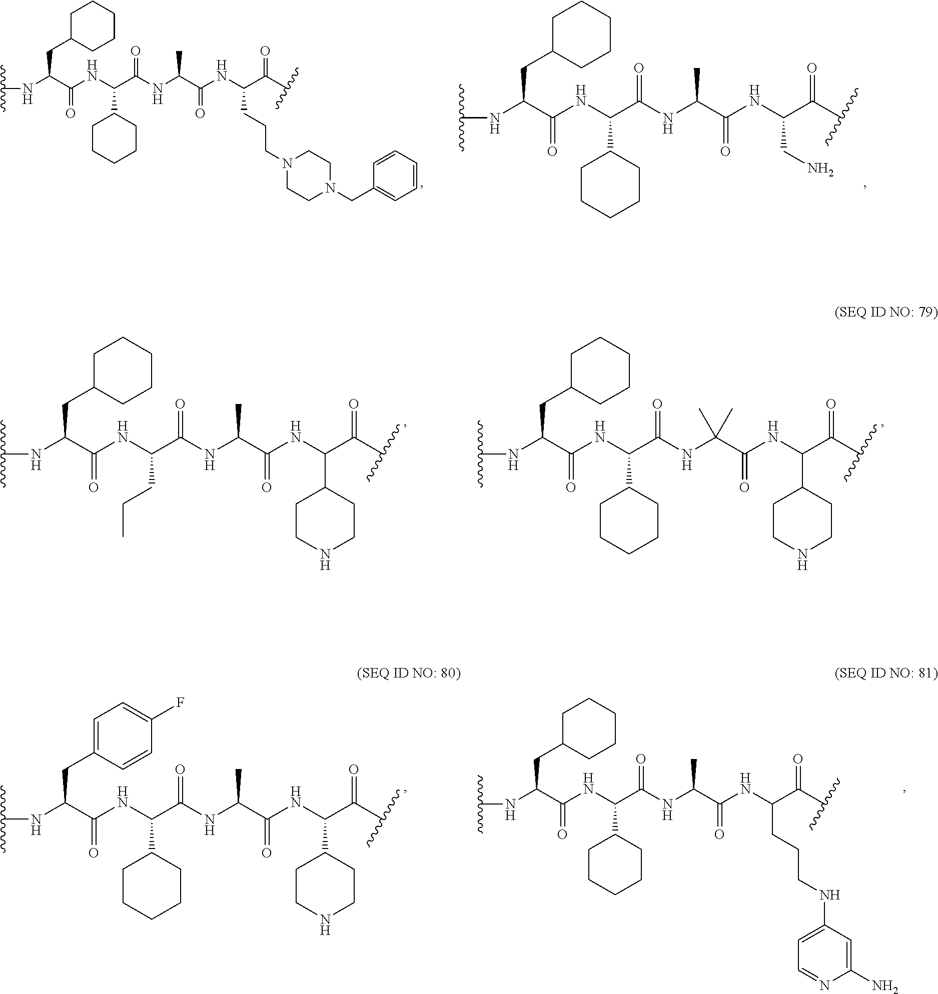

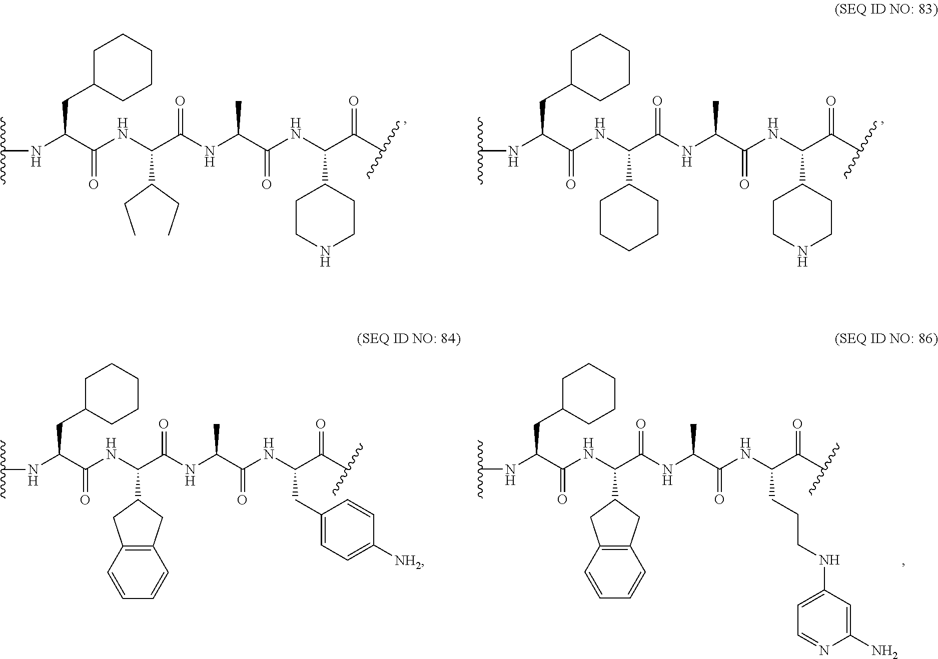 [SMILES: *N[C@@H](CC1CCCCC1)C(=O)N[C@H](C(=O)N[C@@H](C)C(=O)N[C@H](C(*)=O)C1CCNCC1)C(CC)CC ; *N[C@@H](CC1CCCCC1)C(=O)N[C@H](C(=O)N[C@@H](C)C(=O)N[C@H](C(*)=O)C1CCNCC1)C1CCCCC1 ; *N[C@@H](CC1CCCCC1)C(=O)N[C@H](C(=O)N[C@@H](C)C(=O)N[C@@H](Cc1ccc(N)cc1)C(*)=O)C1Cc2ccccc2C1 ; *N[C@@H](CC1CCCCC1)C(=O)N[C@H](C(=O)N[C@@H](C)C(=O)N[C@@H](CCCNc1ccnc(N)c1)C(*)=O)C1Cc2ccccc2C1]

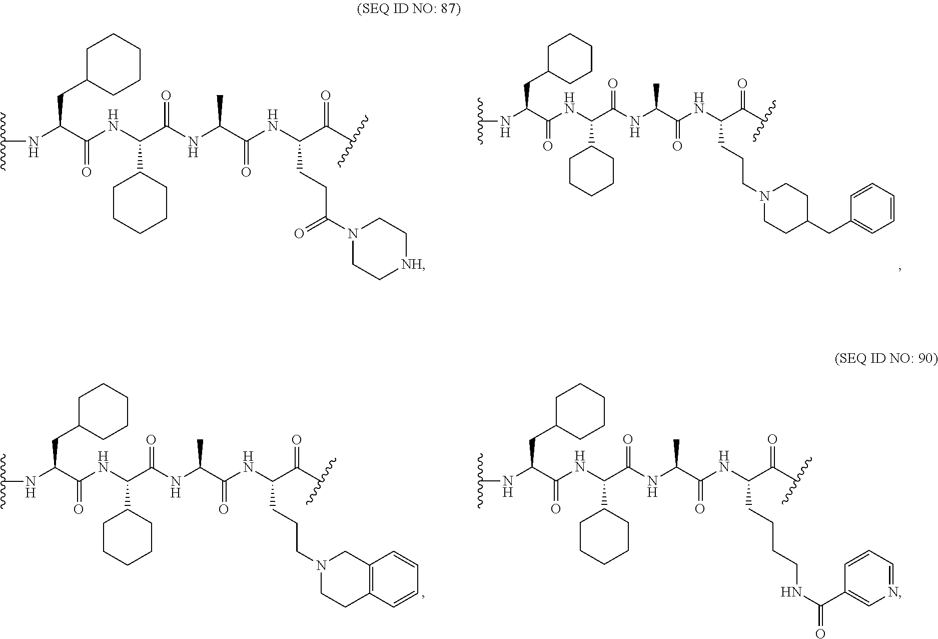 [SMILES: *N[C@@H](CC1CCCCC1)C(=O)N[C@H](C(=O)N[C@@H](C)C(=O)N[C@@H](CCC(=O)N1CCNCC1)C(*)=O)C1CCCCC1 ; *N[C@@H](CC1CCCCC1)C(=O)N[C@H](C(=O)N[C@@H](C)C(=O)N[C@@H](CCCN1CCC(Cc2ccccc2)CC1)C(*)=O)C1CCCCC1 ; *N[C@@H](CC1CCCCC1)C(=O)N[C@H](C(=O)N[C@@H](C)C(=O)N[C@@H](CCCN1CCc2ccccc2C1)C(*)=O)C1CCCCC1 ; *N[C@@H](CC1CCCCC1)C(=O)N[C@H](C(=O)N[C@@H](C)C(=O)N[C@@H](CCCCNC(=O)c1cccnc1)C(*)=O)C1CCCCC1]

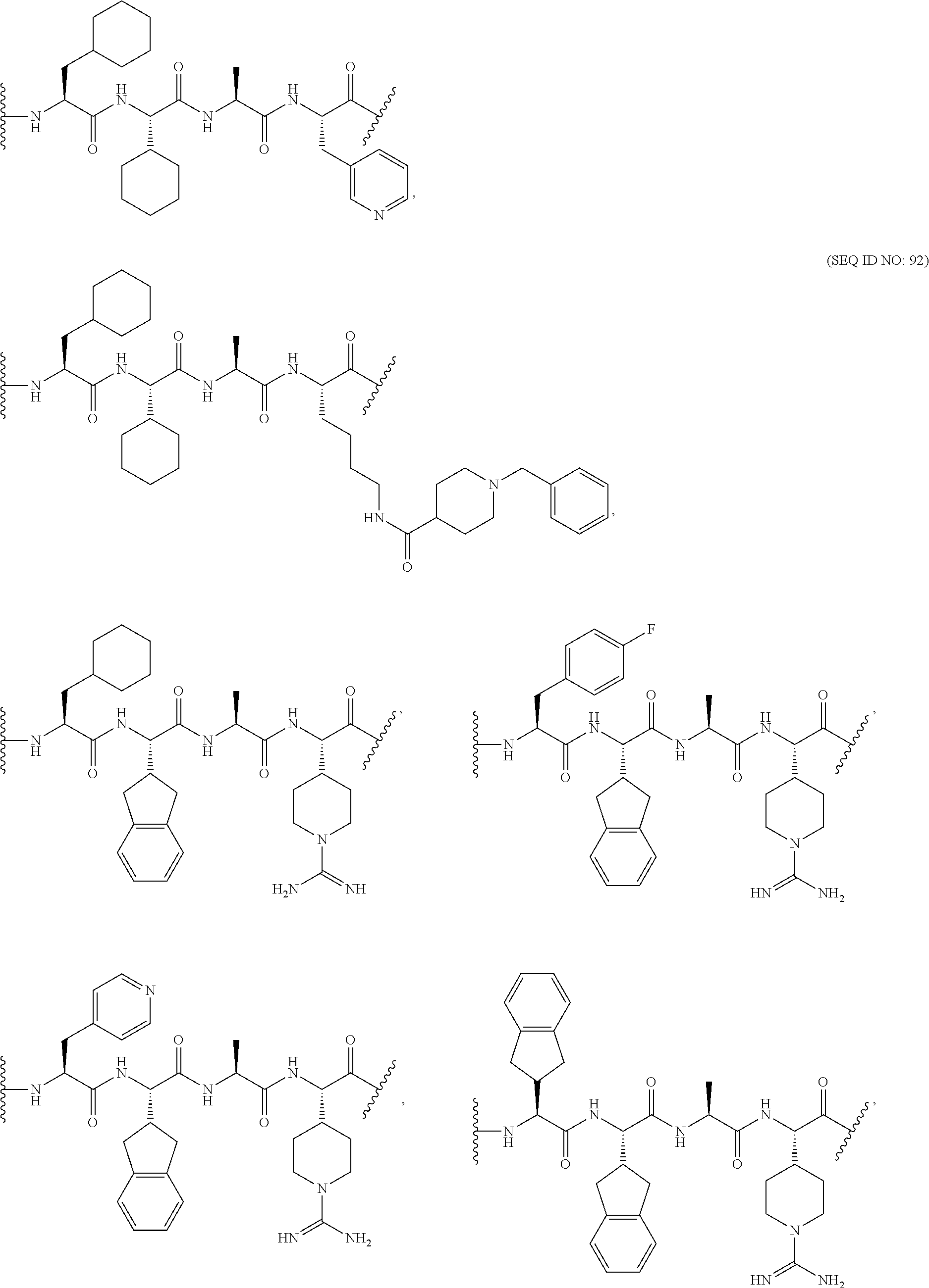

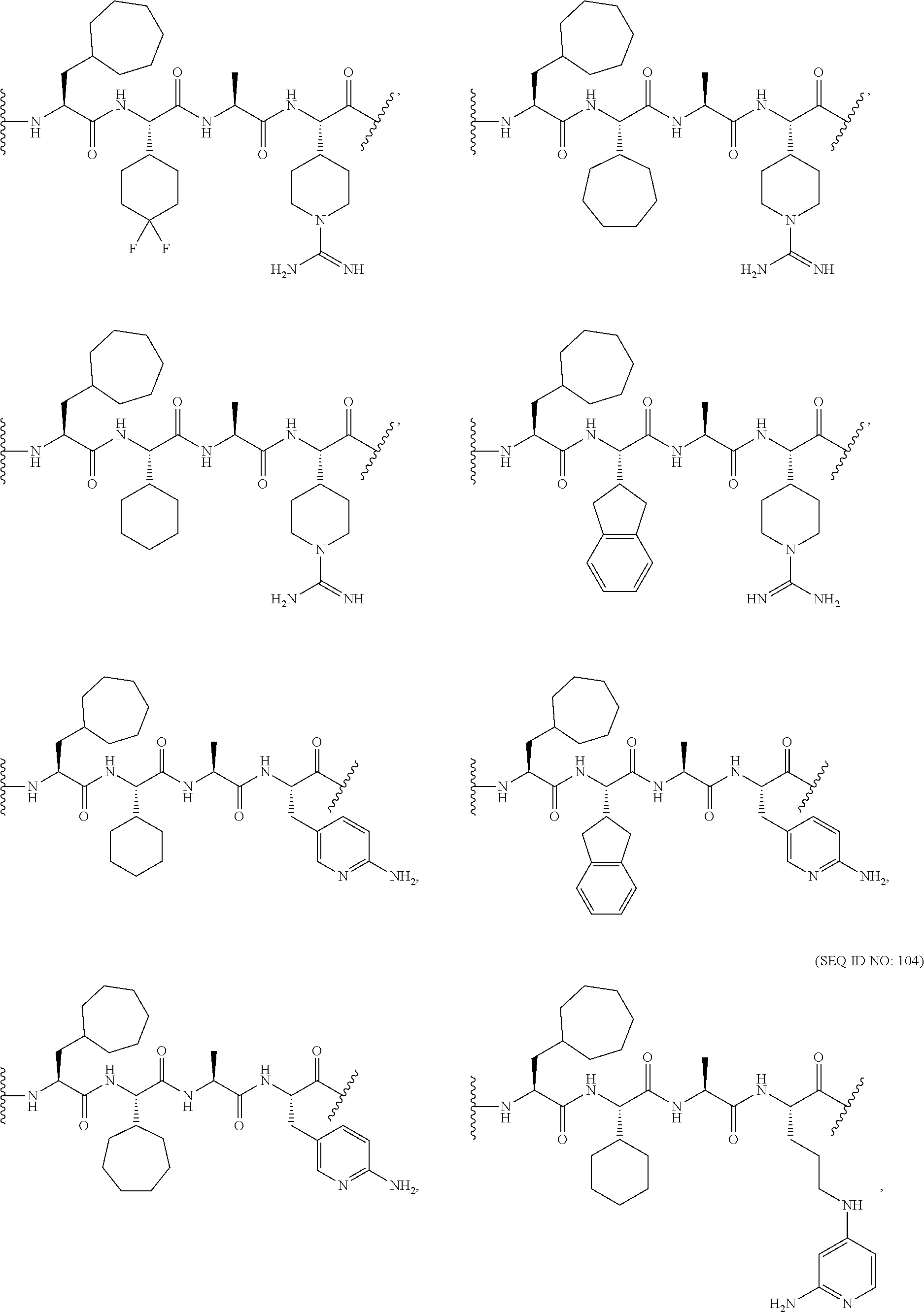

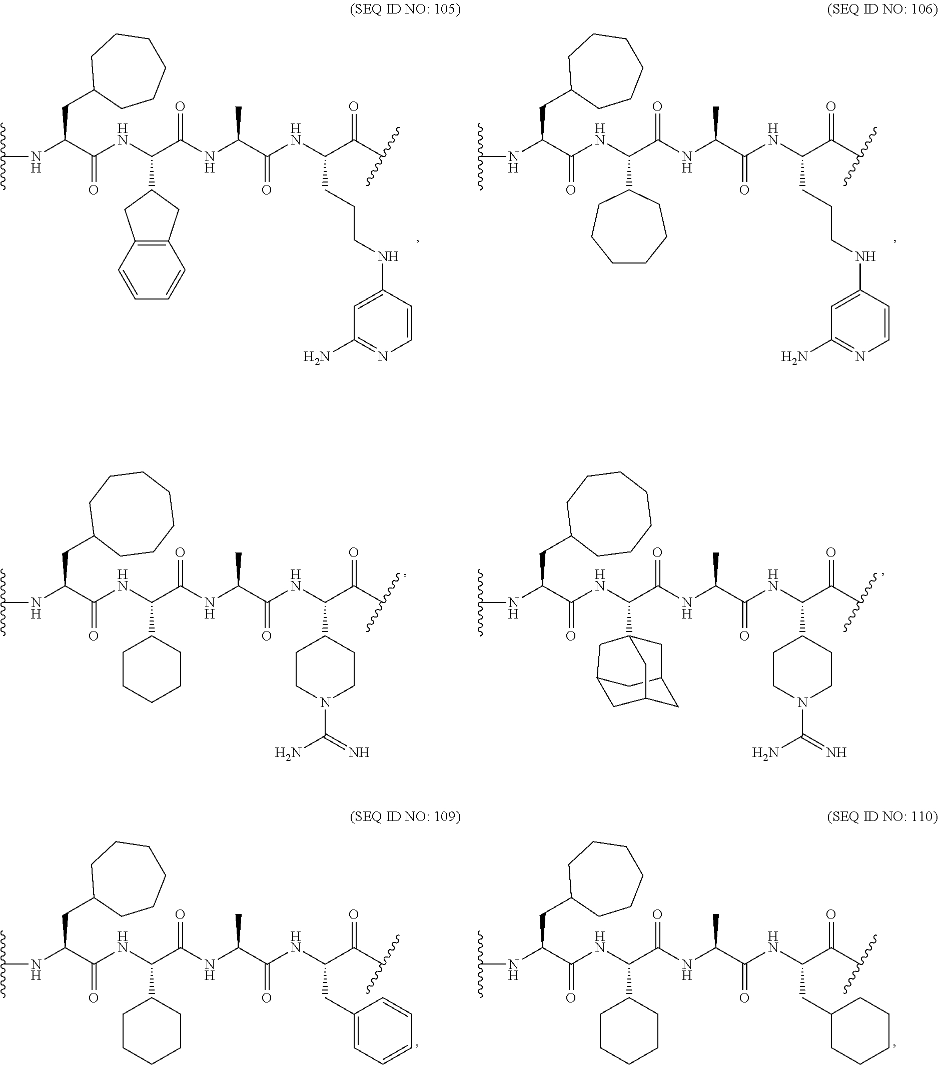

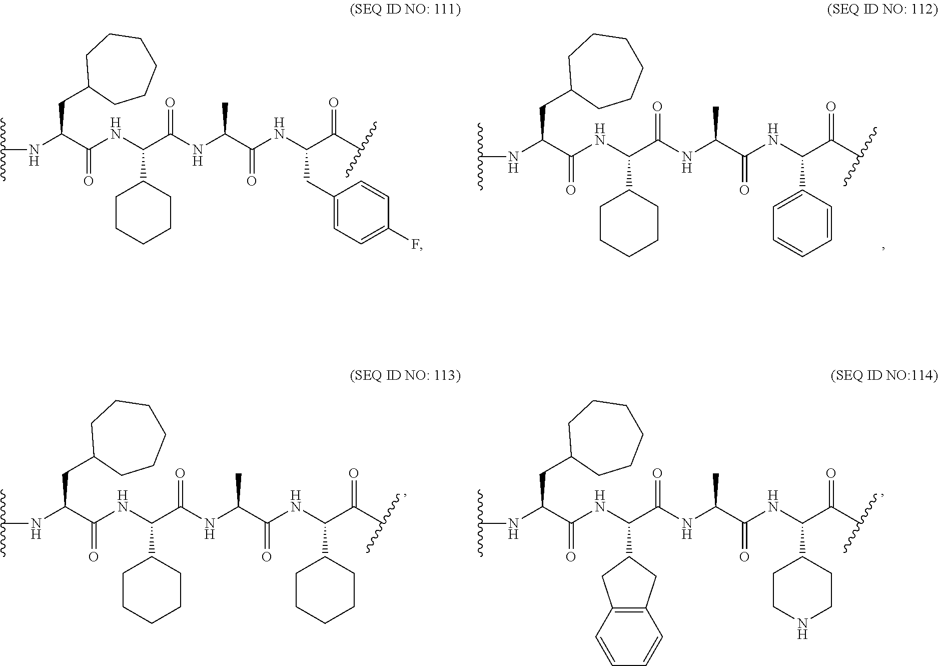 [SMILES: *N[C@@H](CC1CCCCCC1)C(=O)N[C@H](C(=O)N[C@@H](C)C(=O)N[C@@H](Cc1ccc(F)cc1)C(*)=O)C1CCCCC1 ; *N[C@@H](CC1CCCCCC1)C(=O)N[C@H](C(=O)N[C@@H](C)C(=O)N[C@H](C(*)=O)c1ccccc1)C1CCCCC1 ; *N[C@@H](CC1CCCCCC1)C(=O)N[C@H](C(=O)N[C@@H](C)C(=O)N[C@H](C(*)=O)C1CCCCC1)C1CCCCC1 ; *N[C@@H](CC1CCCCCC1)C(=O)N[C@H](C(=O)N[C@@H](C)C(=O)N[C@H](C(*)=O)C1CCNCC1)C1Cc2ccccc2C1]

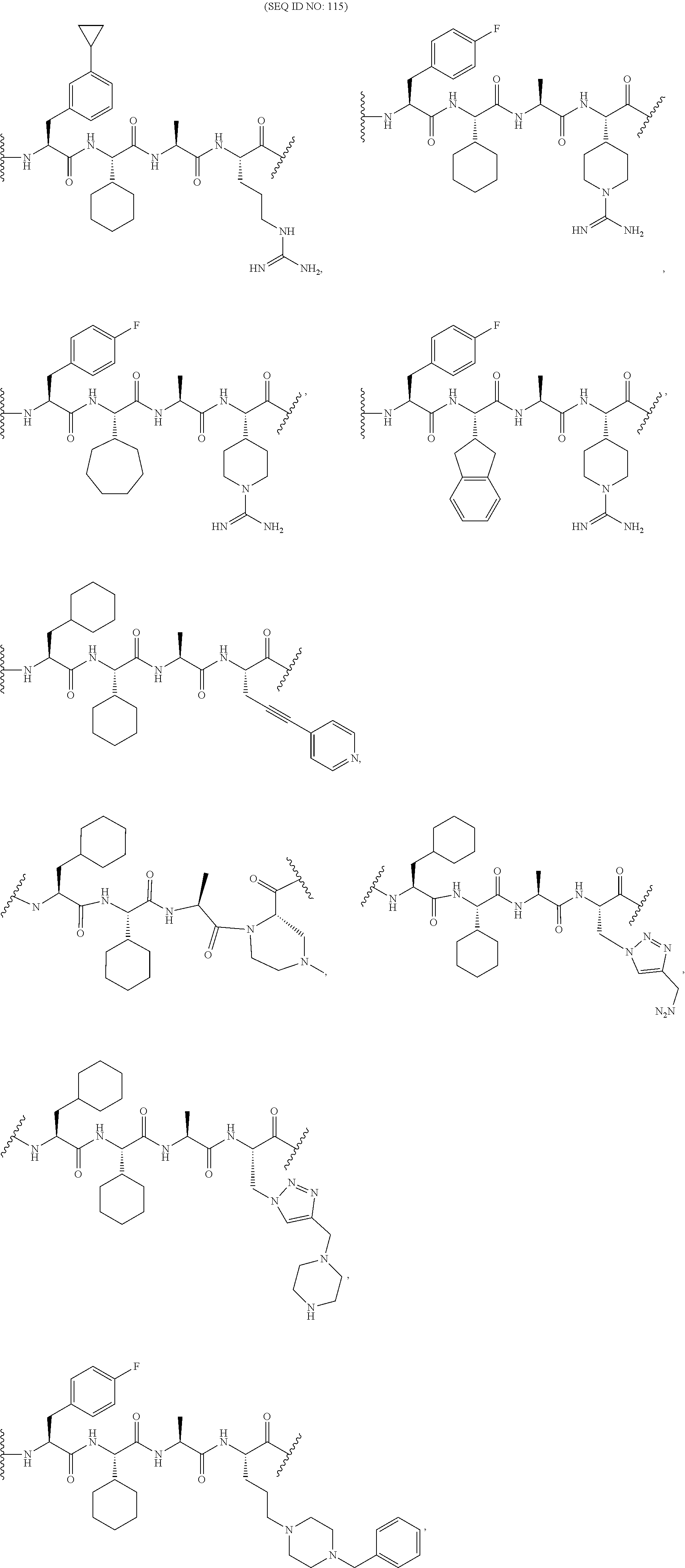

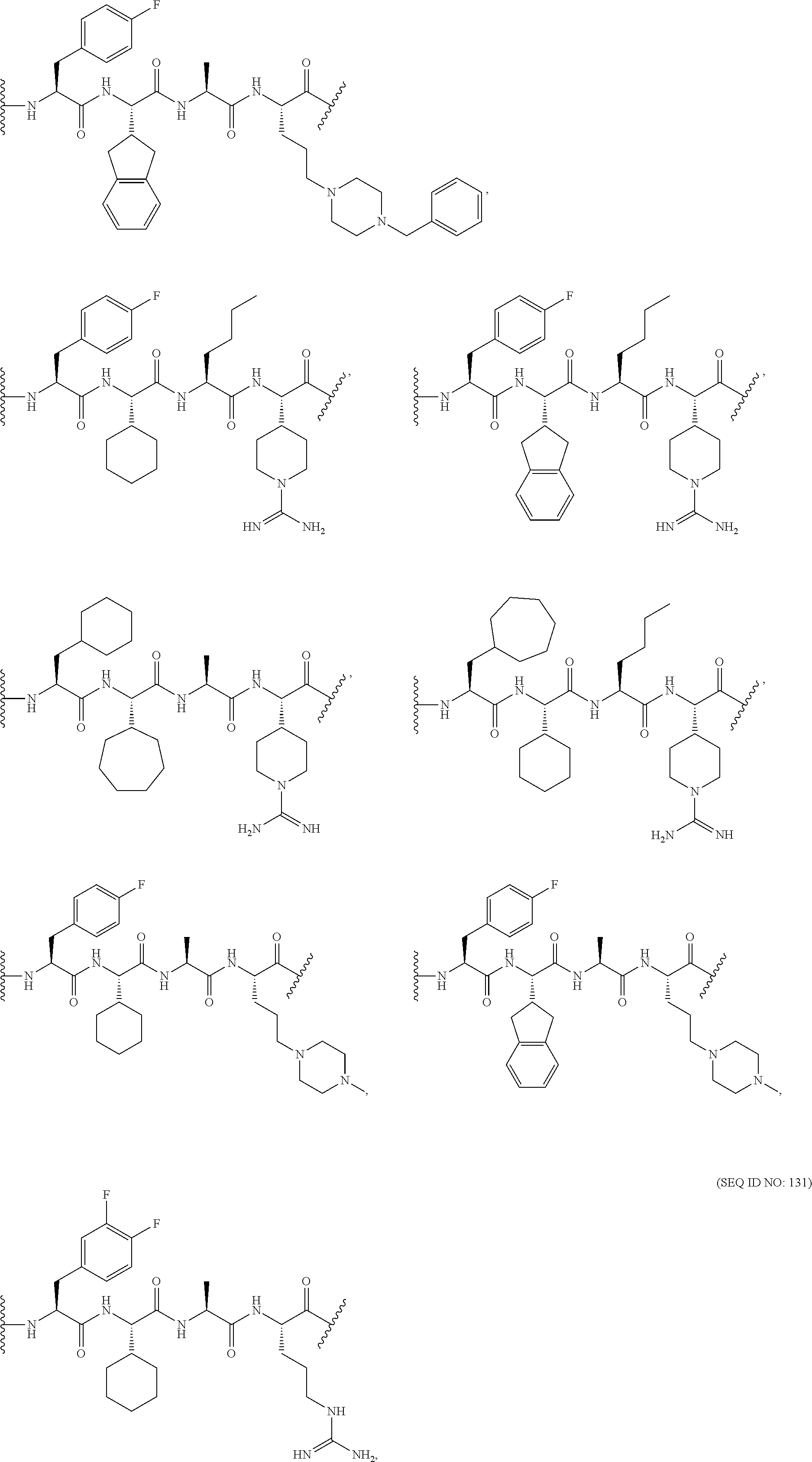

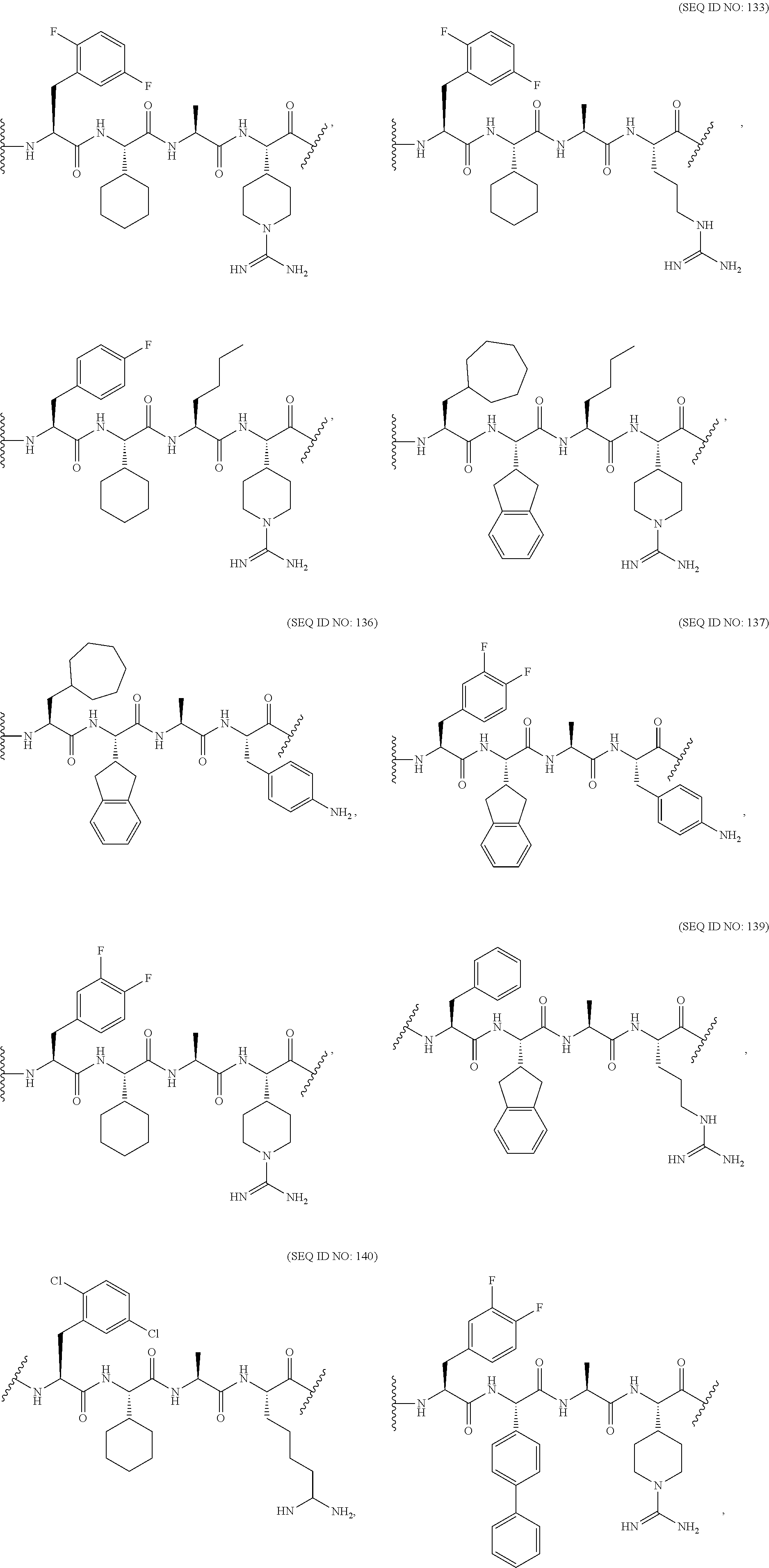

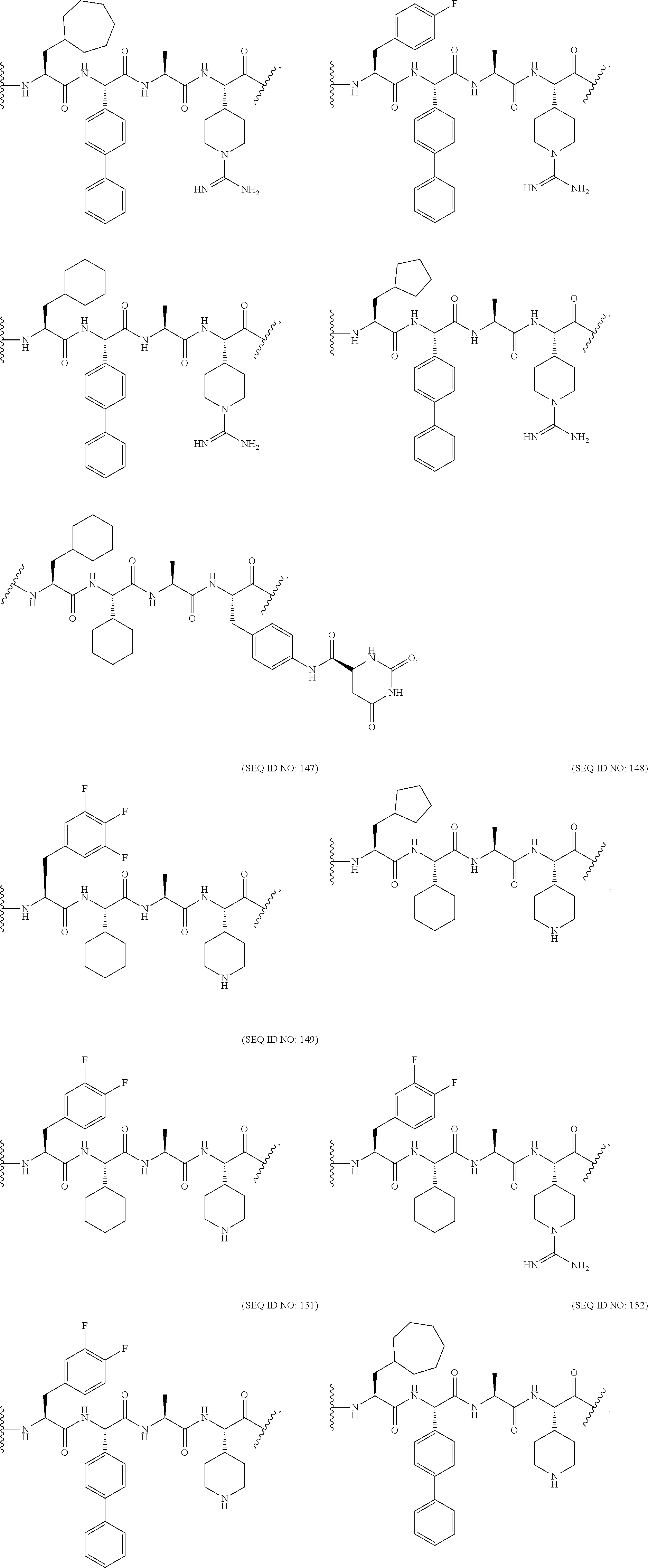

3. Embodiment 111. The compound of any one of embodiments 89 or 92-103, or a pharmaceutically acceptable salt thereof, wherein —X¹—X²—X³—X⁴— is:

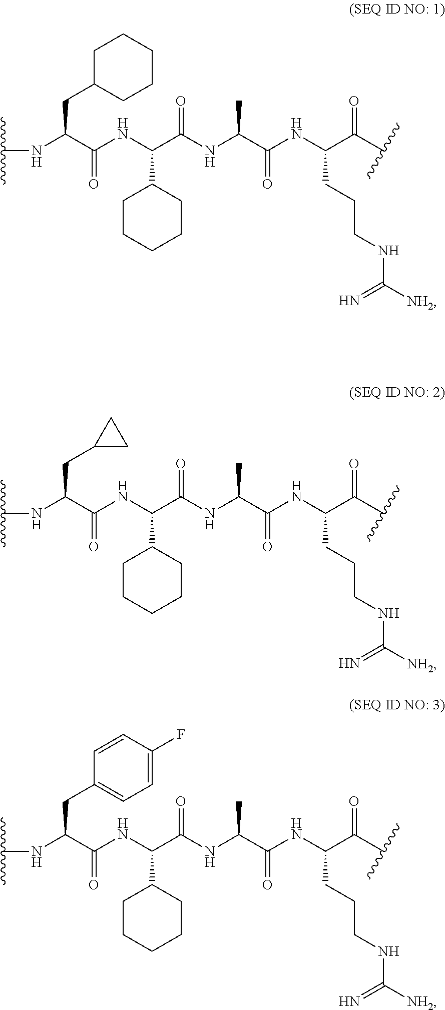 [SMILES: *N[C@@H](CC1CCCCC1)C(=O)N[C@H](C(=O)N[C@@H](C)C(=O)N[C@@H](CCCNC(=N)N)C(*)=O)C1CCCCC1 ; *N[C@@H](CC1CC1)C(=O)N[C@H](C(=O)N[C@@H](C)C(=O)N[C@@H](CCCNC(=N)N)C(*)=O)C1CCCCC1 ; *N[C@@H](Cc1ccc(F)cc1)C(=O)N[C@H](C(=O)N[C@@H](C)C(=O)N[C@@H](CCCNC(=N)N)C(*)=O)C1CCCCC1]

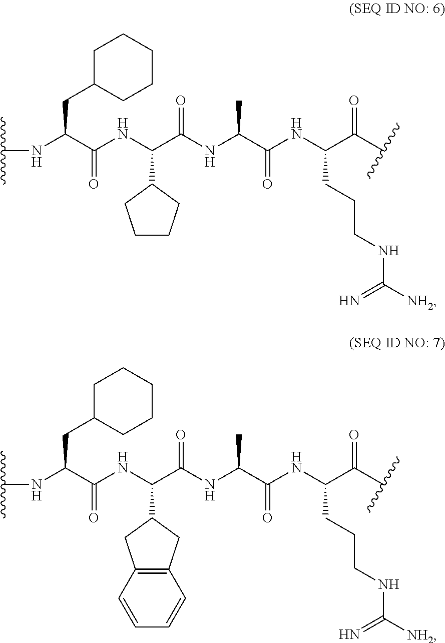 [SMILES: *N[C@@H](CC1CCCCC1)C(=O)N[C@H](C(=O)N[C@@H](C)C(=O)N[C@@H](CCCNC(=N)N)C(*)=O)C1CCCC1 ; *N[C@@H](CC1CCCCC1)C(=O)N[C@H](C(=O)N[C@@H](C)C(=O)N[C@@H](CCCNC(=N)N)C(*)=O)C1Cc2ccccc2C1]

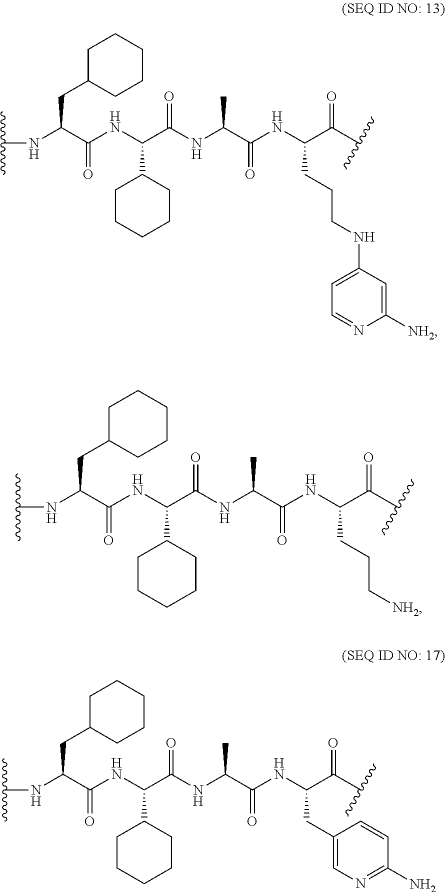 [SMILES: *N[C@@H](CC1CCCCC1)C(=O)N[C@H](C(=O)N[C@@H](C)C(=O)N[C@@H](CCCNc1ccnc(N)c1)C(*)=O)C1CCCCC1 ; *N[C@@H](CC1CCCCC1)C(=O)N[C@H](C(=O)N[C@@H](C)C(=O)N[C@@H](CCCN)C(*)=O)C1CCCCC1 ; *N[C@@H](CC1CCCCC1)C(=O)N[C@H](C(=O)N[C@@H](C)C(=O)N[C@@H](Cc1ccc(N)nc1)C(*)=O)C1CCCCC1]

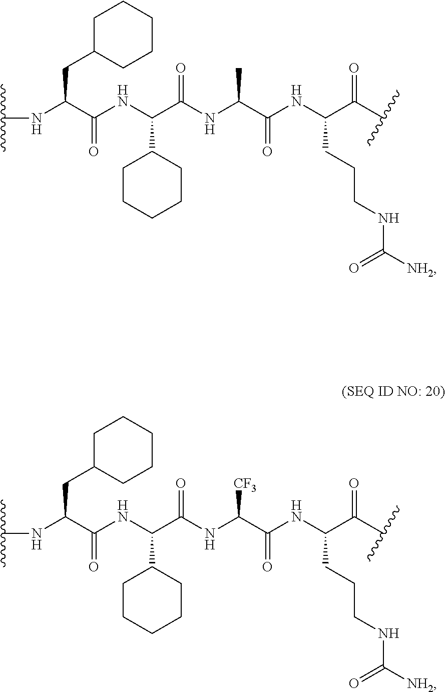 [SMILES: *N[C@@H](CC1CCCCC1)C(=O)N[C@H](C(=O)N[C@@H](C)C(=O)N[C@@H](CCCNC(N)=O)C(*)=O)C1CCCCC1 ; *N[C@@H](CC1CCCCC1)C(=O)N[C@H](C(=O)N[C@H](C(=O)N[C@@H](CCCNC(N)=O)C(*)=O)C(F)(F)F)C1CCCCC1]

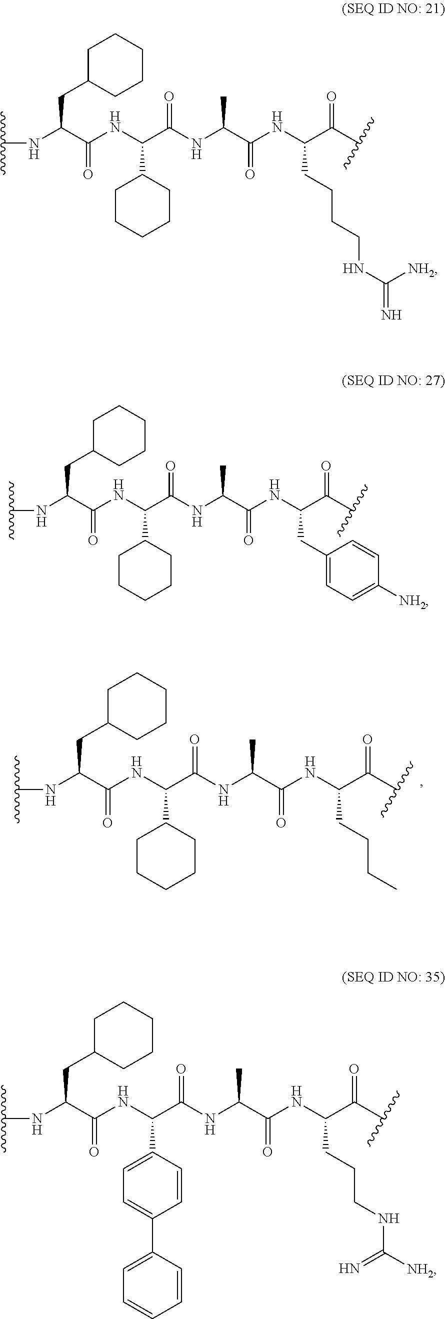 [SMILES: *N[C@@H](CC1CCCCC1)C(=O)N[C@H](C(=O)N[C@@H](C)C(=O)N[C@@H](CCCCNC(=N)N)C(*)=O)C1CCCCC1 ; *N[C@@H](CC1CCCCC1)C(=O)N[C@H](C(=O)N[C@@H](C)C(=O)N[C@@H](Cc1ccc(N)cc1)C(*)=O)C1CCCCC1 ; *N[C@@H](CC1CCCCC1)C(=O)N[C@H](C(=O)N[C@@H](C)C(=O)N[C@@H](CCCC)C(*)=O)C1CCCCC1 ; *N[C@@H](CC1CCCCC1)C(=O)N[C@H](C(=O)N[C@@H](C)C(=O)N[C@@H](CCCNC(=N)N)C(*)=O)c1ccc(-c2ccccc2)cc1]

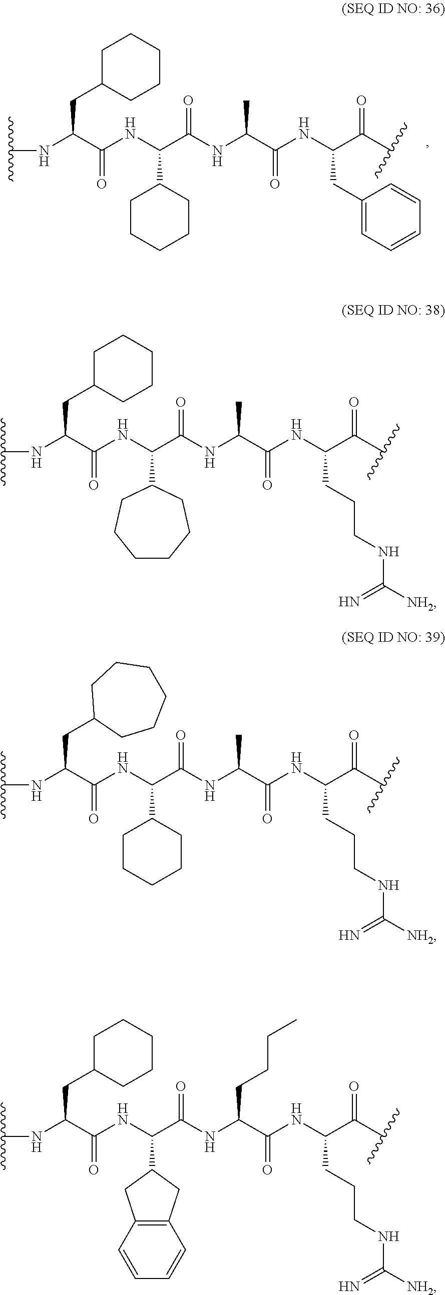 [SMILES: *N[C@@H](CC1CCCCC1)C(=O)N[C@H](C(=O)N[C@@H](C)C(=O)N[C@@H](Cc1ccccc1)C(*)=O)C1CCCCC1 ; *N[C@@H](CC1CCCCC1)C(=O)N[C@H](C(=O)N[C@@H](C)C(=O)N[C@@H](CCCNC(=N)N)C(*)=O)C1CCCCCC1 ; *N[C@@H](CC1CCCCCC1)C(=O)N[C@H](C(=O)N[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](CCCC)C(=O)N[C@@H](CCCNC(=N)N)C(*)=O)C1Cc2ccccc2C1]

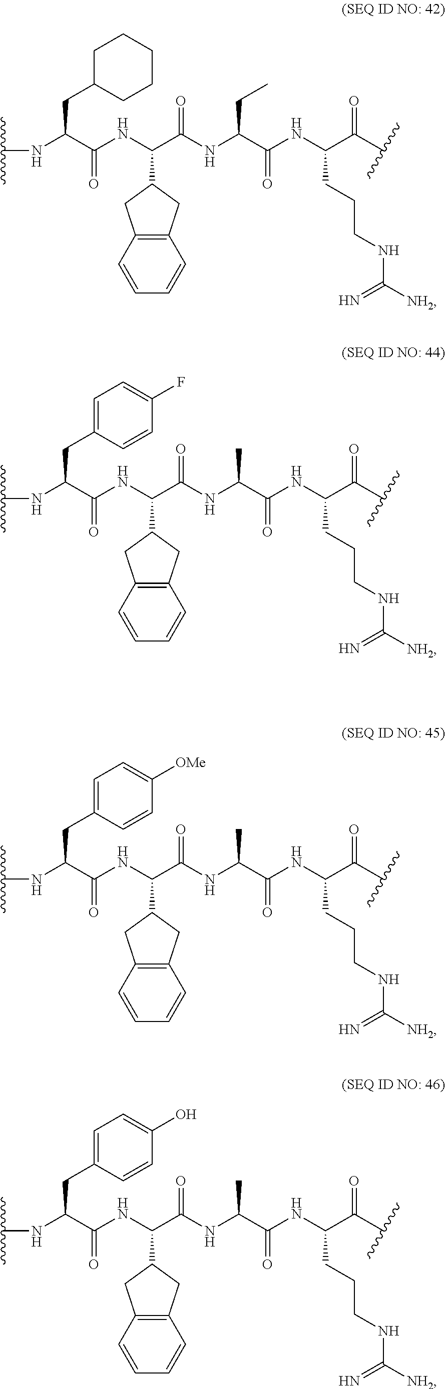 [SMILES: *N[C@@H](CC1CCCCC1)C(=O)N[C@H](C(=O)N[C@@H](CC)C(=O)N[C@@H](CCCNC(=N)N)C(*)=O)C1Cc2ccccc2C1 ; *N[C@@H](Cc1ccc(F)cc1)C(=O)N[C@H](C(=O)N[C@@H](C)C(=O)N[C@@H](CCCNC(=N)N)C(*)=O)C1Cc2ccccc2C1 ; *N[C@@H](Cc1ccc(OC)cc1)C(=O)N[C@H](C(=O)N[C@@H](C)C(=O)N[C@@H](CCCNC(=N)N)C(*)=O)C1Cc2ccccc2C1 ; *N[C@@H](Cc1ccc(O)cc1)C(=O)N[C@H](C(=O)N[C@@H](C)C(=O)N[C@@H](CCCNC(=N)N)C(*)=O)C1Cc2ccccc2C1]

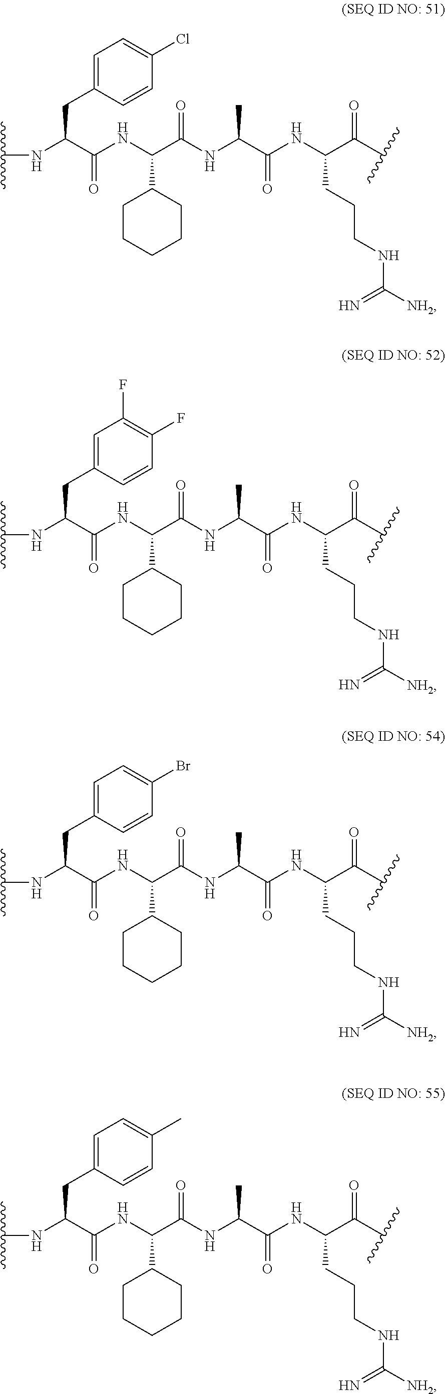 [SMILES: *N[C@@H](Cc1ccc(Cl)cc1)C(=O)N[C@H](C(=O)N[C@@H](C)C(=O)N[C@@H](CCCNC(=N)N)C(*)=O)C1CCCCC1 ; *N[C@@H](Cc1ccc(F)c(F)c1)C(=O)N[C@H](C(=O)N[C@@H](C)C(=O)N[C@@H](CCCNC(=N)N)C(*)=O)C1CCCCC1 ; *N[C@@H](Cc1ccc(Br)cc1)C(=O)N[C@H](C(=O)N[C@@H](C)C(=O)N[C@@H](CCCNC(=N)N)C(*)=O)C1CCCCC1 ; *N[C@@H](Cc1ccc(C)cc1)C(=O)N[C@H](C(=O)N[C@@H](C)C(=O)N[C@@H](CCCNC(=N)N)C(*)=O)C1CCCCC1]

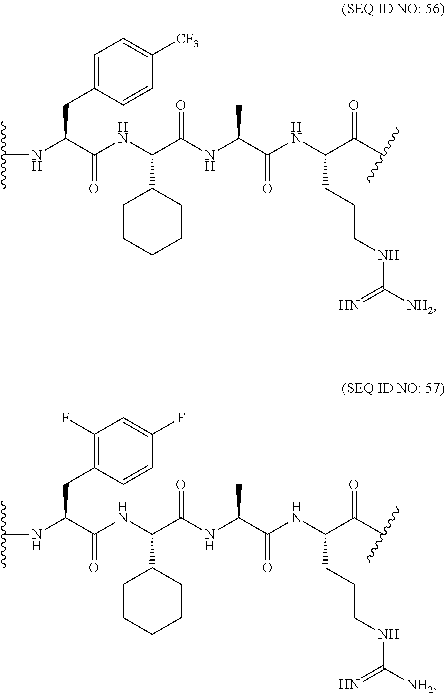 [SMILES: *N[C@@H](Cc1ccc(C(F)(F)F)cc1)C(=O)N[C@H](C(=O)N[C@@H](C)C(=O)N[C@@H](CCCNC(=N)N)C(*)=O)C1CCCCC1 ; *N[C@@H](Cc1ccc(F)cc1F)C(=O)N[C@H](C(=O)N[C@@H](C)C(=O)N[C@@H](CCCNC(=N)N)C(*)=O)C1CCCCC1]

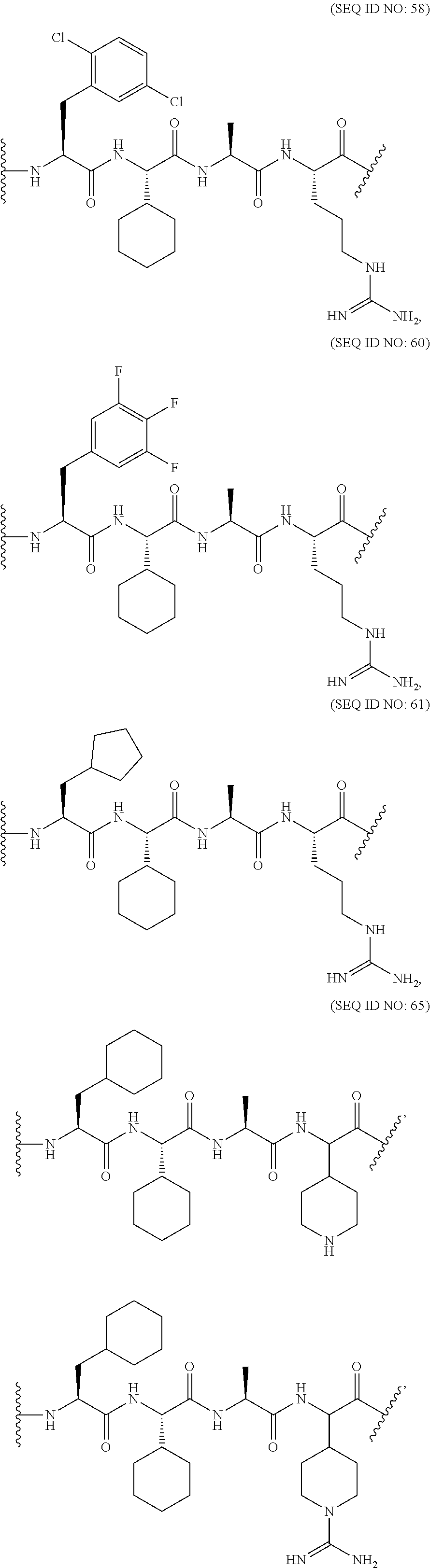 [SMILES: *N[C@@H](Cc1cc(Cl)ccc1Cl)C(=O)N[C@H](C(=O)N[C@@H](C)C(=O)N[C@@H](CCCNC(=N)N)C(*)=O)C1CCCCC1 ; *N[C@@H](Cc1cc(F)c(F)c(F)c1)C(=O)N[C@H](C(=O)N[C@@H](C)C(=O)N[C@@H](CCCNC(=N)N)C(*)=O)C1CCCCC1 ; *N[C@@H](CC1CCCC1)C(=O)N[C@H](C(=O)N[C@@H](C)C(=O)N[C@@H](CCCNC(=N)N)C(*)=O)C1CCCCC1 ; *N[C@@H](CC1CCCCC1)C(=O)N[C@H](C(=O)N[C@@H](C)C(=O)NC(C(*)=O)C1CCNCC1)C1CCCCC1 ; *N[C@@H](CC1CCCCC1)C(=O)N[C@H](C(=O)N[C@@H](C)C(=O)NC(C(*)=O)C1CCN(C(=N)N)CC1)C1CCCCC1]

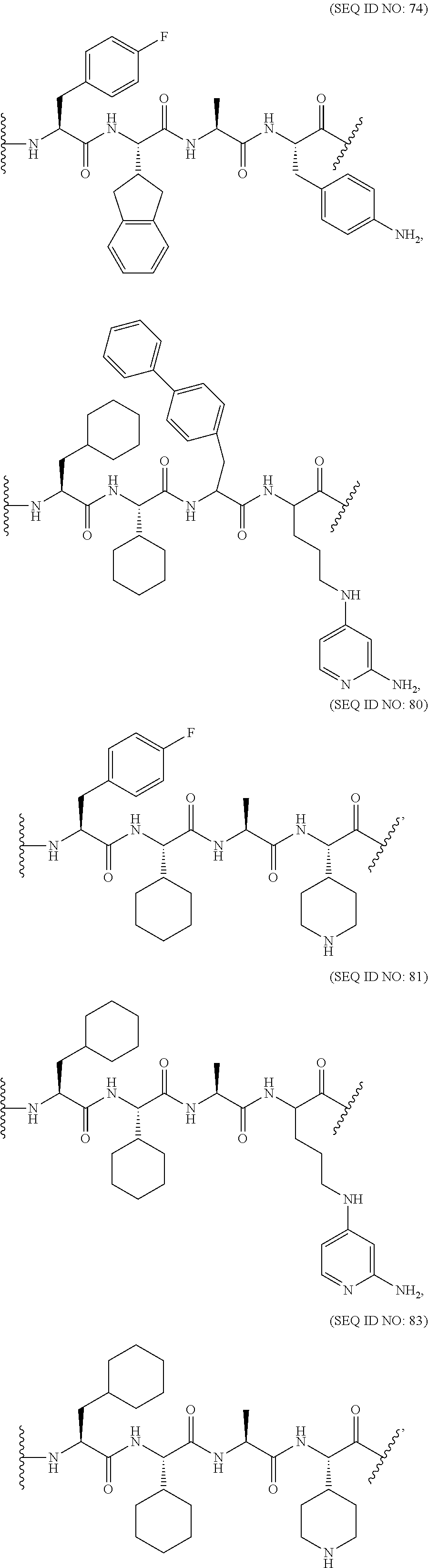 [SMILES: *N[C@@H](Cc1ccc(F)cc1)C(=O)N[C@H](C(=O)N[C@@H](C)C(=O)N[C@@H](Cc1ccc(N)cc1)C(*)=O)C1Cc2ccccc2C1 ; *N[C@@H](CC1CCCCC1)C(=O)N[C@H](C(=O)NC(Cc1ccc(-c2ccccc2)cc1)C(=O)NC(CCCNc1ccnc(N)c1)C(*)=O)C1CCCCC1 ; *N[C@@H](Cc1ccc(F)cc1)C(=O)N[C@H](C(=O)N[C@@H](C)C(=O)N[C@H](C(*)=O)C1CCNCC1)C1CCCCC1 ; *N[C@@H](CC1CCCCC1)C(=O)N[C@H](C(=O)N[C@@H](C)C(=O)NC(CCCNc1ccnc(N)c1)C(*)=O)C1CCCCC1 ; *N[C@@H](CC1CCCCC1)C(=O)N[C@H](C(=O)N[C@@H](C)C(=O)N[C@H](C(*)=O)C1CCNCC1)C1CCCCC1]

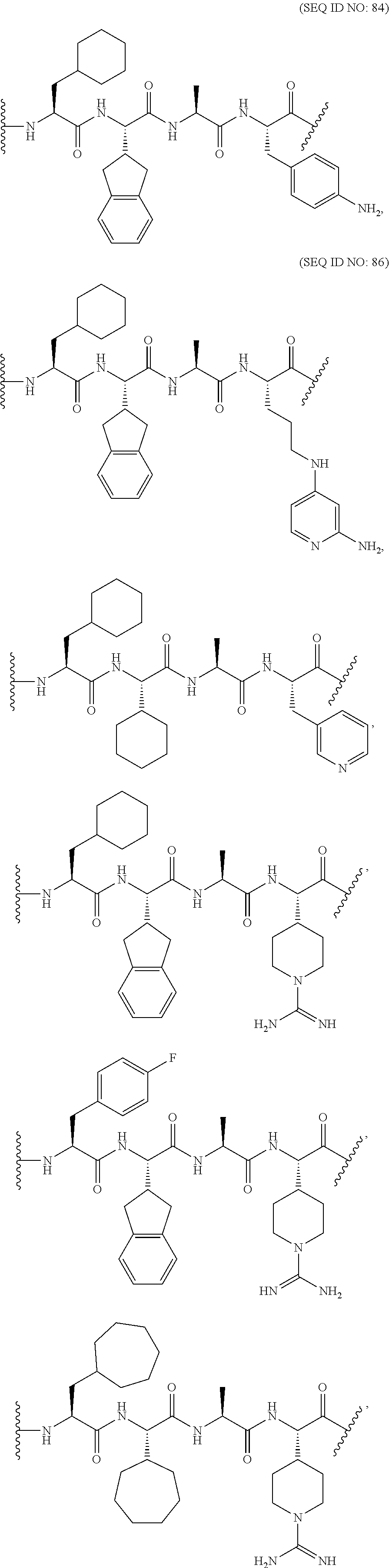

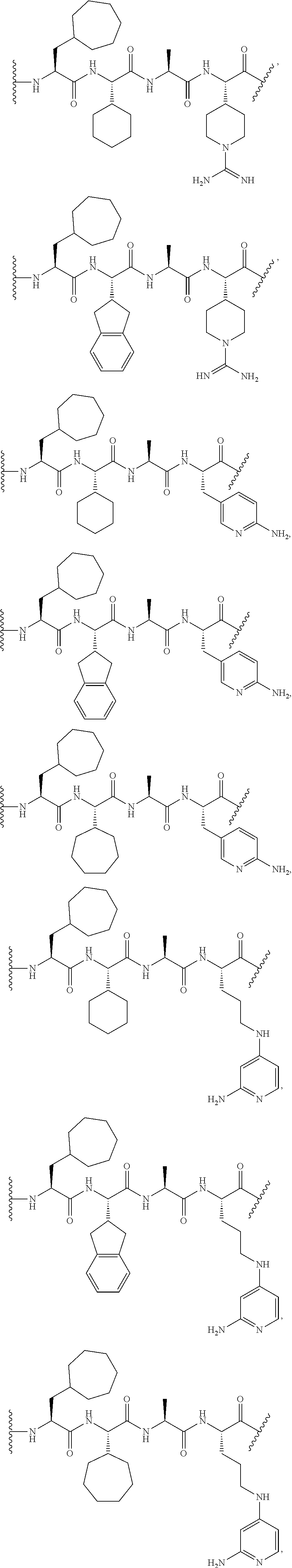

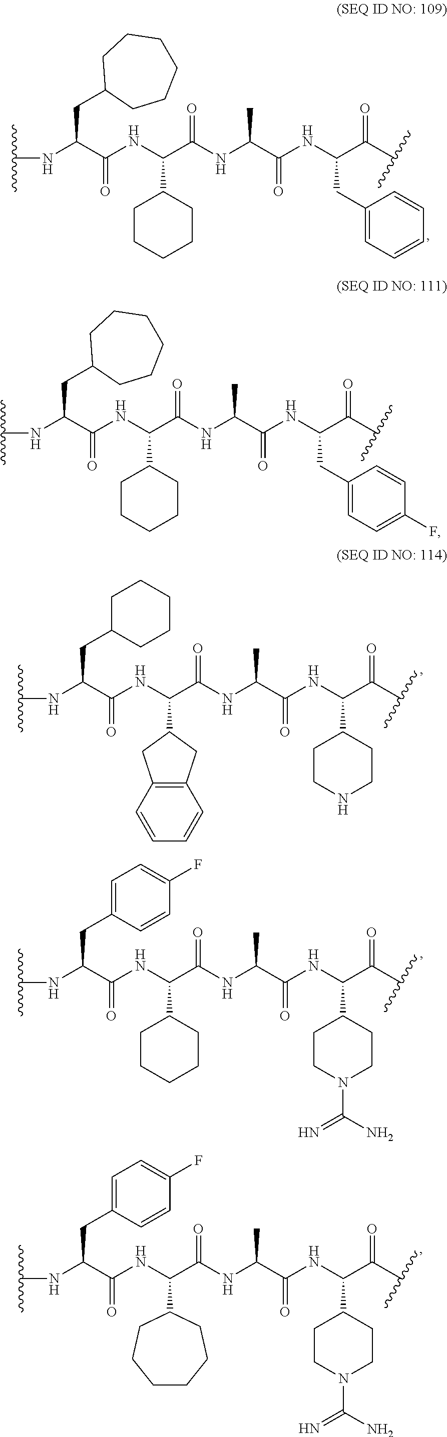

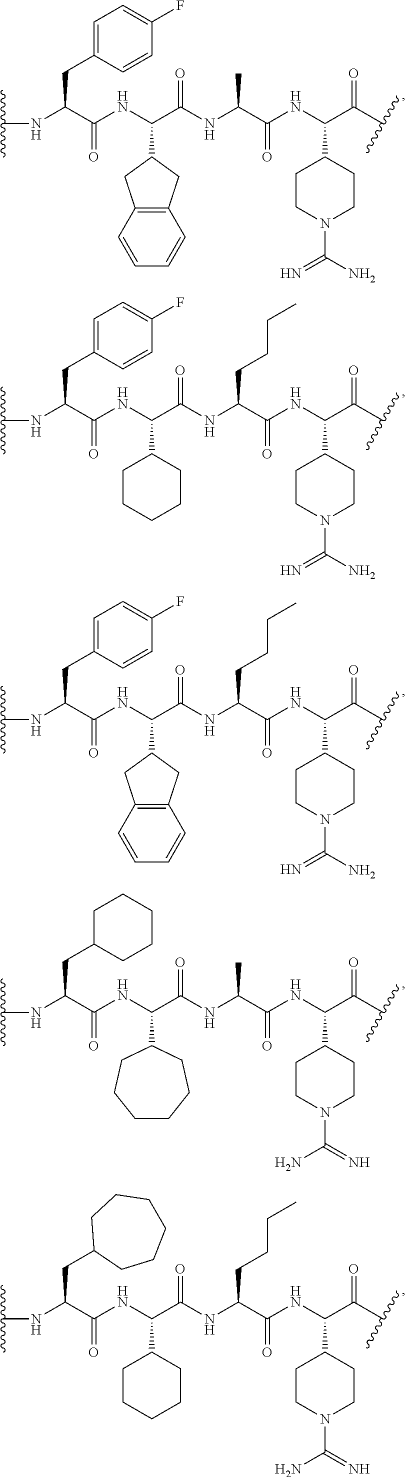

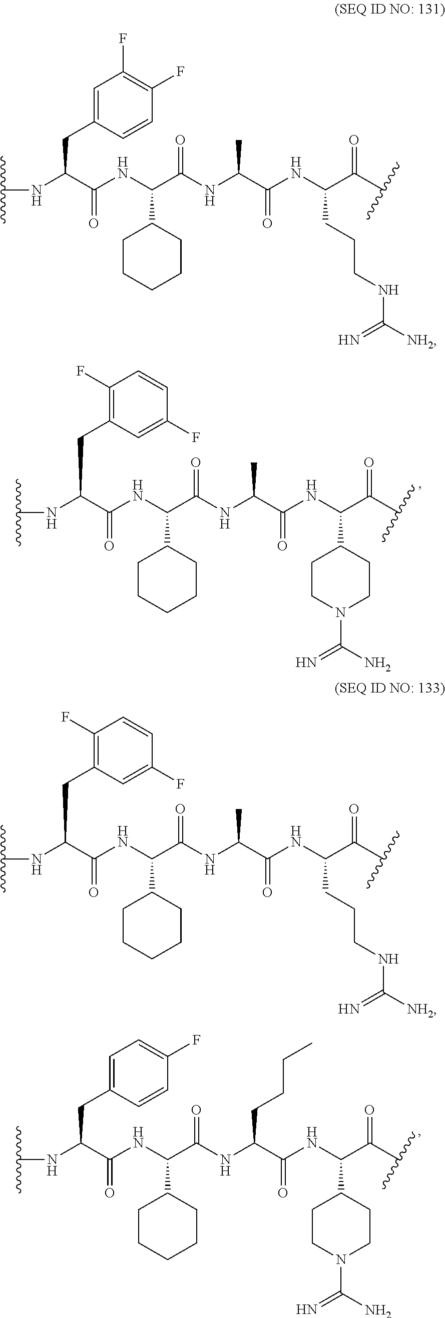 [SMILES: *N[C@@H](Cc1ccc(F)c(F)c1)C(=O)N[C@H](C(=O)N[C@@H](C)C(=O)N[C@@H](CCCNC(=N)N)C(*)=O)C1CCCCC1 ; *N[C@@H](Cc1cc(F)ccc1F)C(=O)N[C@H](C(=O)N[C@@H](C)C(=O)N[C@H](C(*)=O)C1CCN(C(=N)N)CC1)C1CCCCC1 ; *N[C@@H](Cc1cc(F)ccc1F)C(=O)N[C@H](C(=O)N[C@@H](C)C(=O)N[C@@H](CCCNC(=N)N)C(*)=O)C1CCCCC1 ; *N[C@@H](Cc1ccc(F)cc1)C(=O)N[C@H](C(=O)N[C@@H](CCCC)C(=O)N[C@H](C(*)=O)C1CCN(C(=N)N)CC1)C1CCCCC1]

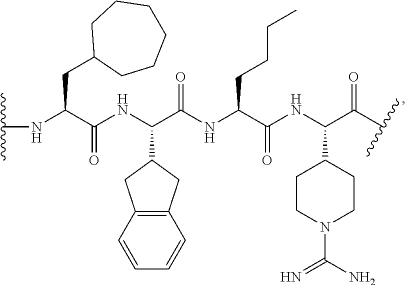 [SMILES: *N[C@@H](CC1CCCCCC1)C(=O)N[C@H](C(=O)N[C@@H](CCCC)C(=O)N[C@H](C(*)=O)C1CCN(C(=N)N)CC1)C1Cc2ccccc2C1]

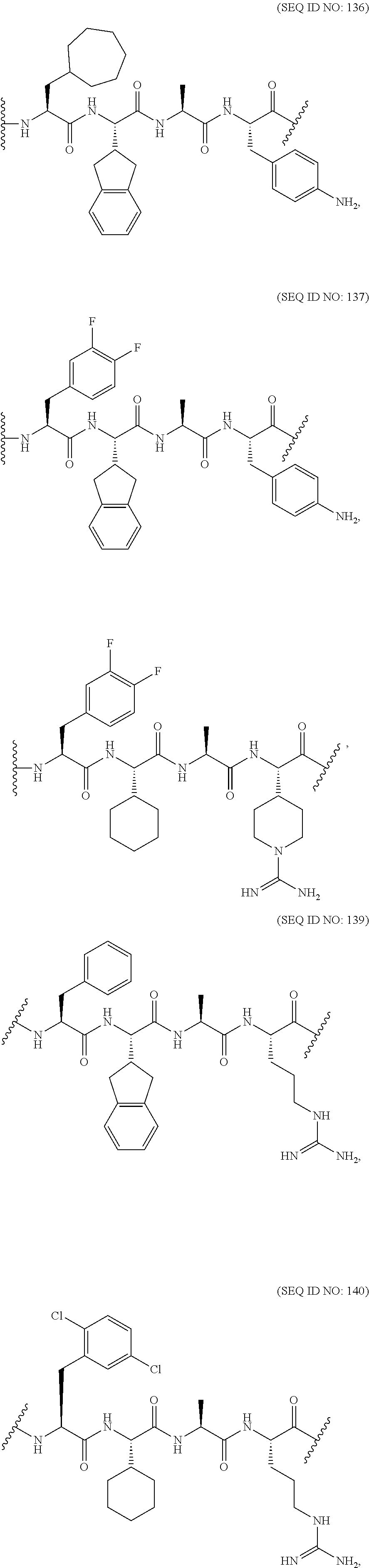 [SMILES: *N[C@@H](CC1CCCCCC1)C(=O)N[C@H](C(=O)N[C@@H](C)C(=O)N[C@@H](Cc1ccc(N)cc1)C(*)=O)C1Cc2ccccc2C1 ; *N[C@@H](Cc1ccc(F)c(F)c1)C(=O)N[C@H](C(=O)N[C@@H](C)C(=O)N[C@@H](Cc1ccc(N)cc1)C(*)=O)C1Cc2ccccc2C1 ; *N[C@@H](Cc1ccc(F)c(F)c1)C(=O)N[C@H](C(=O)N[C@@H](C)C(=O)N[C@H](C(*)=O)C1CCN(C(=N)N)CC1)C1CCCCC1 ; *N[C@@H](Cc1ccccc1)C(=O)N[C@H](C(=O)N[C@@H](C)C(=O)N[C@@H](CCCNC(=N)N)C(*)=O)C1Cc2ccccc2C1 ; *N[C@@H](Cc1cc(Cl)ccc1Cl)C(=O)N[C@H](C(=O)N[C@@H](C)C(=O)N[C@@H](CCCNC(=N)N)C(*)=O)C1CCCCC1]

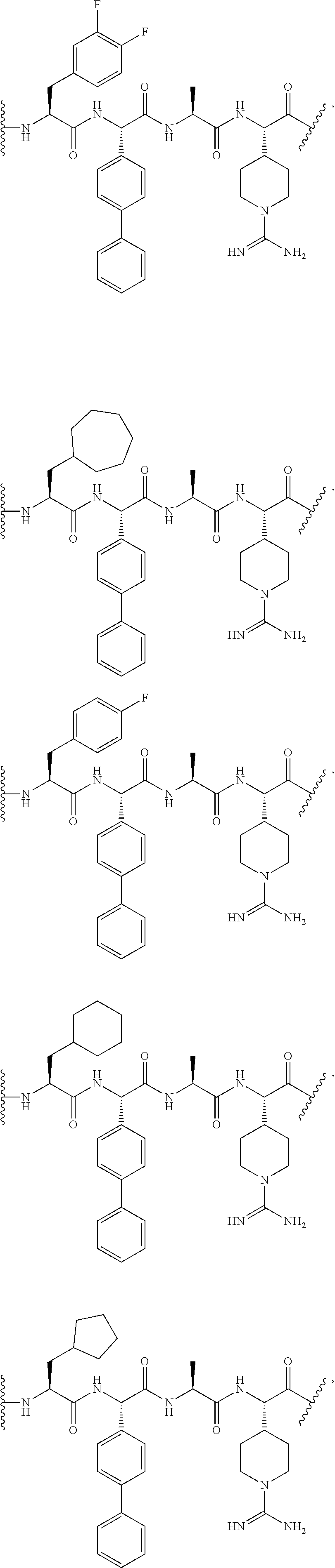

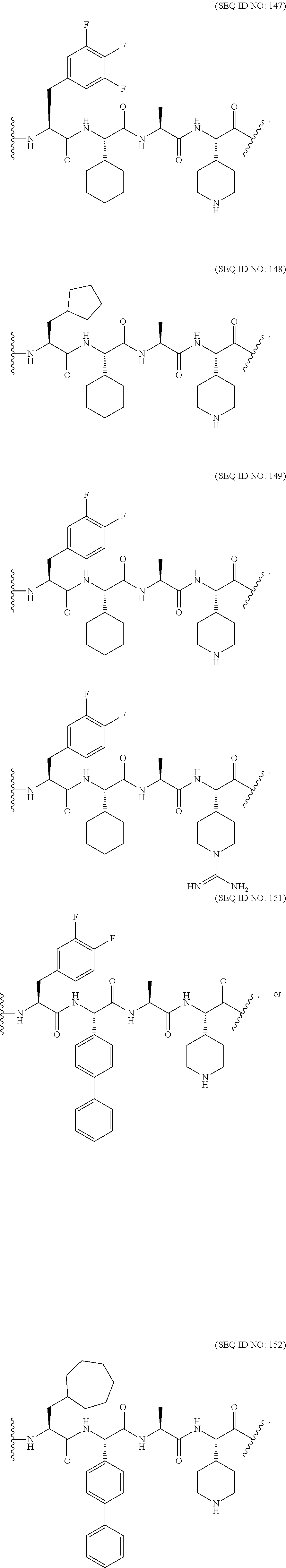

Embodiment 112. The compound of any one of embodiments 89-90 or 92-111, or a pharmaceutically acceptable salt thereof, wherein R^{a}—X¹—X²—X³—X⁴— is:

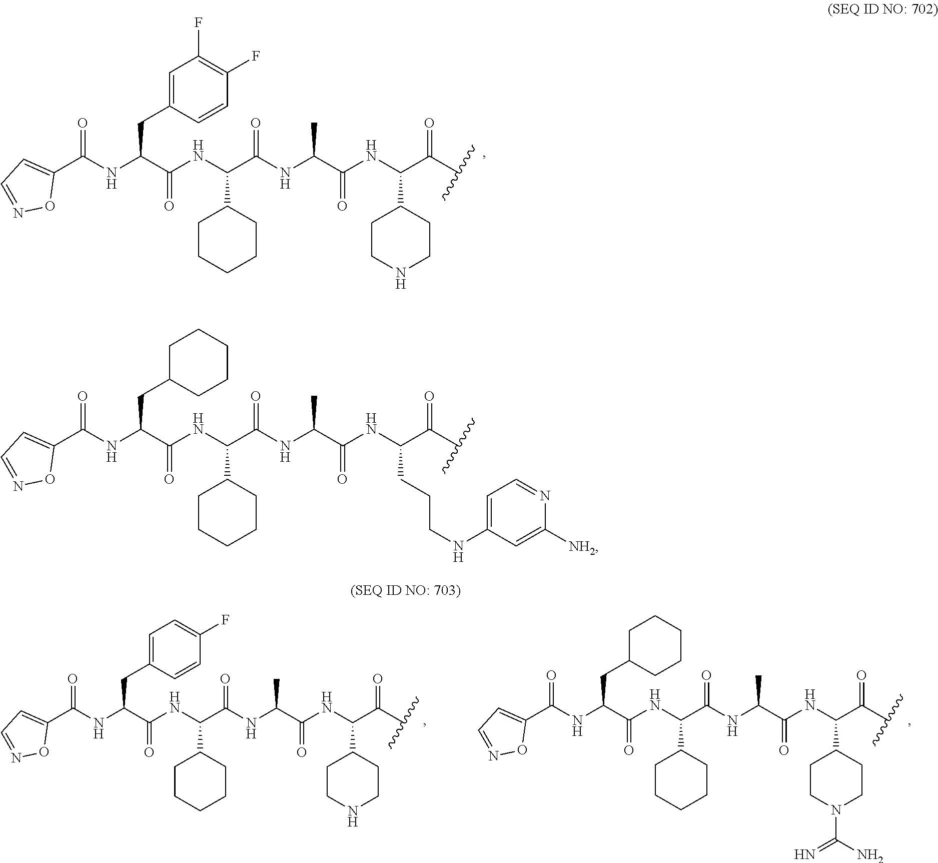 [SMILES: *C(=O)[C@@H](NC(=O)[C@H](C)NC(=O)[C@@H](NC(=O)[C@H](Cc1ccc(F)c(F)c1)NC(=O)c1ccno1)C1CCCCC1)C1CCNCC1 ; *C(=O)[C@H](CCCNc1ccnc(N)c1)NC(=O)[C@H](C)NC(=O)[C@@H](NC(=O)[C@H](CC1CCCCC1)NC(=O)c1ccno1)C1CCCCC1 ; *C(=O)[C@@H](NC(=O)[C@H](C)NC(=O)[C@@H](NC(=O)[C@H](Cc1ccc(F)cc1)NC(=O)c1ccno1)C1CCCCC1)C1CCNCC1 ; *C(=O)[C@@H](NC(=O)[C@H](C)NC(=O)[C@@H](NC(=O)[C@H](CC1CCCCC1)NC(=O)c1ccno1)C1CCCCC1)C1CCN(C(=N)N)CC1]

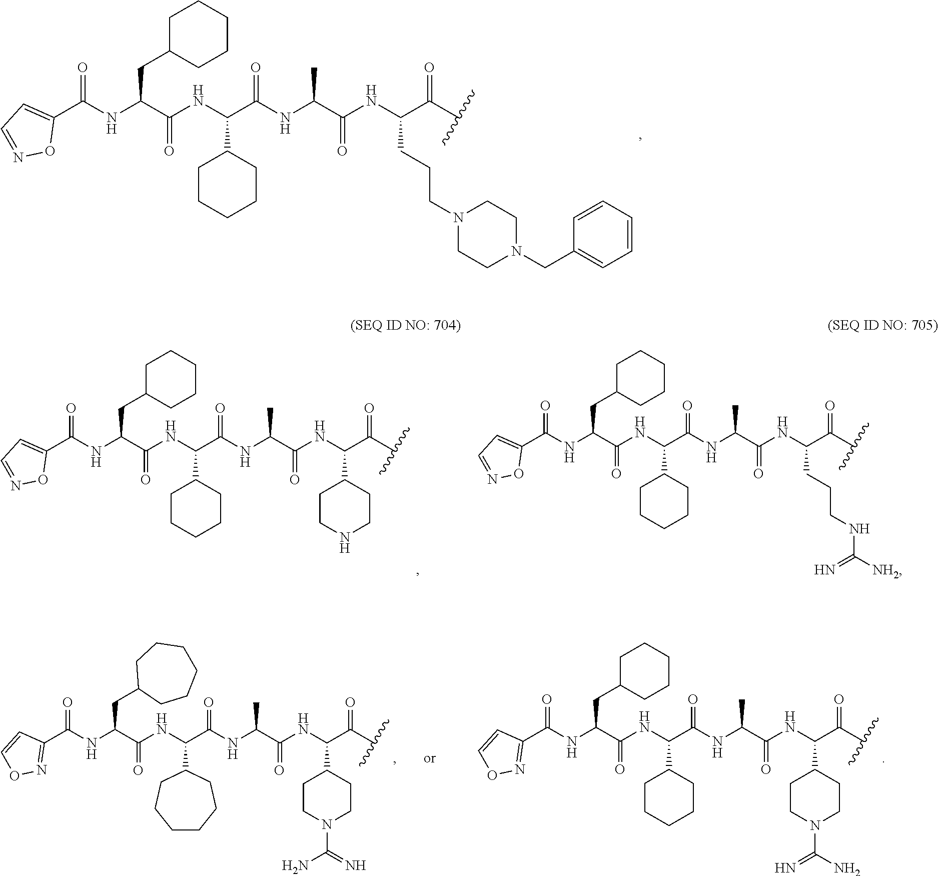

Embodiment 113. The compound of any one of embodiments 89-112, or a pharmaceutically acceptable salt thereof, wherein X⁵ is -L⁵-, (S)-alanine (Ala), (S)-lysine (Lys), (S)-ornithine (Orn), or (S)-(4-fluorobenzamido)methylglycine.

Embodiment 114. The compound of any one of embodiments 89-112, or a pharmaceutically acceptable salt thereof, wherein X⁵ is -L⁵-.

Embodiment 115. The compound of any one of embodiments 89-112, or a pharmaceutically acceptable salt thereof, wherein X⁵ is (S)-lysine (Lys) or (S)-ornithine (Orn).

Embodiment 116. The compound of any one of embodiments 89-112, or a pharmaceutically acceptable salt thereof, wherein X⁵ is (R)-lysine (Lys) or (R)-ornithine (Orn).

Embodiment 117. The compound of any one of embodiments 89-112, or a pharmaceutically acceptable salt thereof, wherein X⁵ is (S)-(4-fluorobenzamido)methylglycine.

Embodiment 118. The compound of any one of embodiments 89-112, or a pharmaceutically acceptable salt thereof, wherein X⁵ is N6-palmitoyl-(S)-lysine, N6-(15-carboxypentadecanoyl)-(S)-Lys, N6-(16-oxo-16-((2-(N—N—N-trimethyl-amino)ethyl)amino)hexadecanoyl)-(S)-Lys, N6-(4-fluorobenzoyl)-(S)-lysine, (S)-β³-homolysine, (S)-3-amino-alanine (H-Dap-OH), 4-fluoro-(S)-phenylalanine (Phe(4-F)), 4-iodo-(S)-phenylalanine (Phe(4-I)), 4-nitro-(S)-phenylalanine (Phe(4-NO₂)), (S)-(4-fluorobenzamido)methylglycine, 4-aminomethylphenylalanine (Phe(4-CH₂NH₂)), (S)-7-hydroxy-1,2,3,4-tetrahydroisoquinoline-3-carboxylic acid, (S)-proline, (S)-hydroxyproline (Hyp), (S)-piperidine-2-carboxylic acid, (S)-isoindoline-1-carboxylic acid, or (S)-(4-(aminomethyl)-1H-1,2λ²,3λ²-triazol-1-yl)methyl)-glycine.

Embodiment 119. The compound of any one of embodiments 89 or 92-118, or a pharmaceutically acceptable salt thereof, wherein
X¹ is

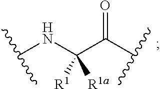

R¹ is -L¹-R^{1b};
L¹ is —CH₂—:
R^{1b} is cyclohexyl, cycloheptyl, or 4-fluorophenyl;
R^{1a} is hydrogen;
X² is

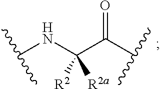

R²— is -L²-R^{2b};
L² is absent;
R^{2b} is cyclohexyl, cycloheptyl, 2,3-dihydro-1H-inden-2-yl, or [1,1′-biphenyl]-4-yl;
R^{2a} is hydrogen;
X³ is (S)-alanine (Ala) or (S)-norleucine (Nle);
X⁴ is (S)-arginine (Arg), (S)-ornithine (Orn), (S)-2-(piperidin-4-yl)-glycine (Gly(piperidin-4-yl)), (S)-2-(I-carbamimidoylpiperidin-4-yl)-glycine (Gly(1-carbamimidoylpiperidin-4-yl)) (S)-4-aminophenylalanine (Phe(4-NH₂)), (S)-(pyridin-3-yl)alanine (3-Pal), (S)-3-(6-aminopyridin-3-yl)alanine ((3-Pal(6-NH₂)), or (S)-2-((2-aminopyridin-4-yl)-λ²-azaneyl)ethyl alanine; and
X⁵ is -L⁵-, alanine (Ala), lysine (Lys), or ornithine (Orn).

Embodiment 120. The compound of any one of embodiments 89-119, or a pharmaceutically acceptable salt thereof, wherein X⁶ is absent.

Embodiment 121. The compound of any one of embodiments 89-119, or a pharmaceutically acceptable salt thereof, wherein X⁶ is -L⁶-.

Embodiment 122. The compound of any one of embodiments 89-119, or a pharmaceutically acceptable salt thereof, wherein X⁶ is (S)-lysine (Lys) or (S)-ornithine (Orn).

Embodiment 123. The compound of any one of embodiments 89-119, or a pharmaceutically acceptable salt thereof, wherein X⁶ is (R)-lysine (Lys) or (R)-ornithine (Orn).

Embodiment 124. The compound of any one of embodiments 89-119, or a pharmaceutically acceptable salt thereof, wherein X⁶ is

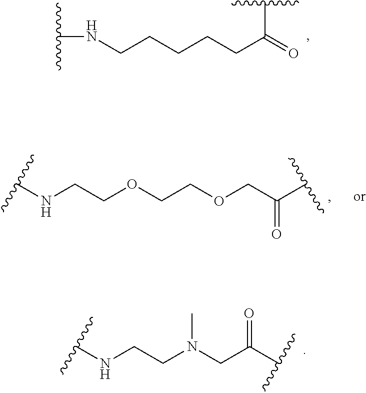

Embodiment 125. The compound of any one of embodiments 89-124, or a pharmaceutically acceptable salt thereof, wherein X⁷ is absent.

Embodiment 126. The compound of any one of embodiments 89-124, or a pharmaceutically acceptable salt thereof, wherein X⁷ is (S)-lysine (Lys) or (S)-ornithine (Orn).

Embodiment 127. The compound of any one of embodiments 89-126, or a pharmaceutically acceptable salt thereof, wherein R^{a} is —C(═O)—R¹⁰, wherein R¹¹ is a heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl, indolizinyl, indolyl, benzofuranyl, benzothiophenyl, indazolyl, benzimidazolyl, purinyl, quinolizinyl, quinolinyl, isoquinolinyl, cinnolinyl, phthalazinyl, quinazolinyl, quinoxalinyl, 1,8-naphthyridinyl, 1,7-naphthyridinyl, 1,6-naphthyridinyl, 1,5-naphthyridinyl, pteridinyl, and tetrazolo[1,5-a]pyridinyl; and wherein R¹⁰ is optionally substituted with 1 to 3 substituents that are each independently selected from the group consisting of halo, —C₁-C₆ alkyl, alkoxy, —OH, —NH₂, —NH—C₁-C₆ alkylene-R¹¹, and —O—C₁-C₆ alkylene-R¹¹.

Embodiment 128. The compound of any one of embodiments 89-126, or a pharmaceutically acceptable salt thereof, wherein R^{a} is —C(═O)—R¹⁰, wherein R¹⁰ is a monocyclic heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl; and wherein R¹⁰ is optionally substituted with 1 to 3 substituents that are each independently selected from the group consisting of halo, —C₁-C₆ alkyl, alkoxy, —OH, —NH₂, —NH—C₁-C₆ alkylene-R¹¹, and —O—C₁-C₆ alkylene-R¹¹.

Embodiment 129. The compound of any one of embodiments 89-126, or a pharmaceutically acceptable salt thereof, wherein R^{a} is —C(═O)—R¹⁰, wherein R¹⁰ is a monocyclic heteroaryl selected from the group consisting of pyridinyl, pyrimidinyl, pyrazinyl, and pyridazinyl; and wherein R¹⁰ is optionally substituted with —NH—C₁-C₆ alkylene-R¹¹, and —O—C₁-C₆ alkylene-R¹¹.

Embodiment 130. The compound of any one of embodiments 89-126, or a pharmaceutically acceptable salt thereof, wherein R^{a} is —C(═O)—R¹⁰, wherein R¹⁰ is a phenyl or naphthyl; and wherein R¹⁰ is optionally substituted with 1 to 3 substituents that are each independently selected from the group consisting of halo, C₁-C₆ alkyl, alkoxy, —OH, —NH₂, —NH—C₁-C₆ alkylene-R¹¹, and —O—C₁-C₆ alkylene-R¹¹.

Embodiment 131. The compound of any one of embodiments 89-126, or a pharmaceutically acceptable salt thereof, wherein R^{a} is —C(═O)—R¹⁰, wherein R¹⁰ is a partially unsaturated bicyclic heterocyclic ring; wherein any saturated carbon of R¹⁰ is optionally substituted with oxo (═O); and wherein R¹⁰ is optionally substituted with 1 to 3 substituents that are each independently selected from the group consisting of halo, C₁-C₆ alkyl, alkoxy, —OH, and —NH₂.

Embodiment 132. The compound of any one of embodiments 89-126, or a pharmaceutically acceptable salt thereof, wherein R^{a} is

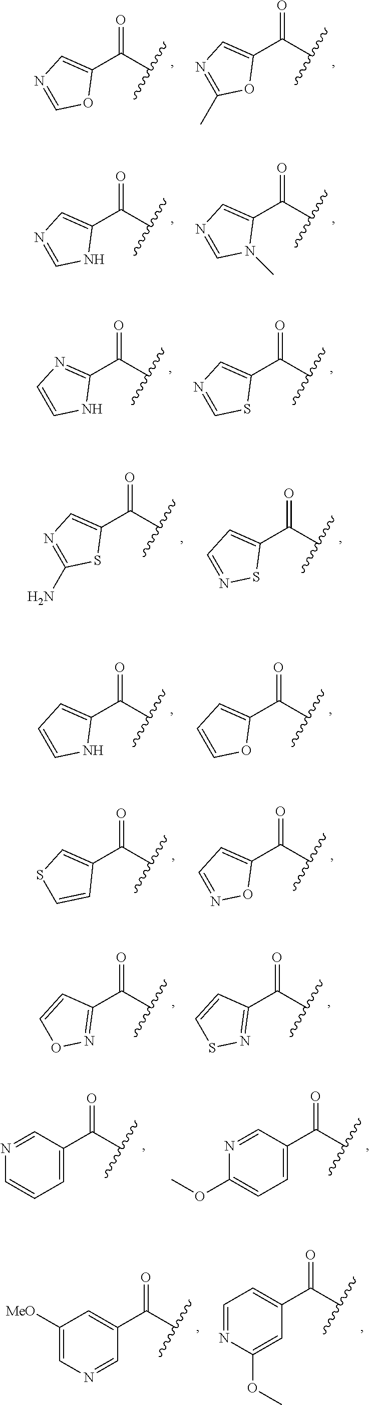

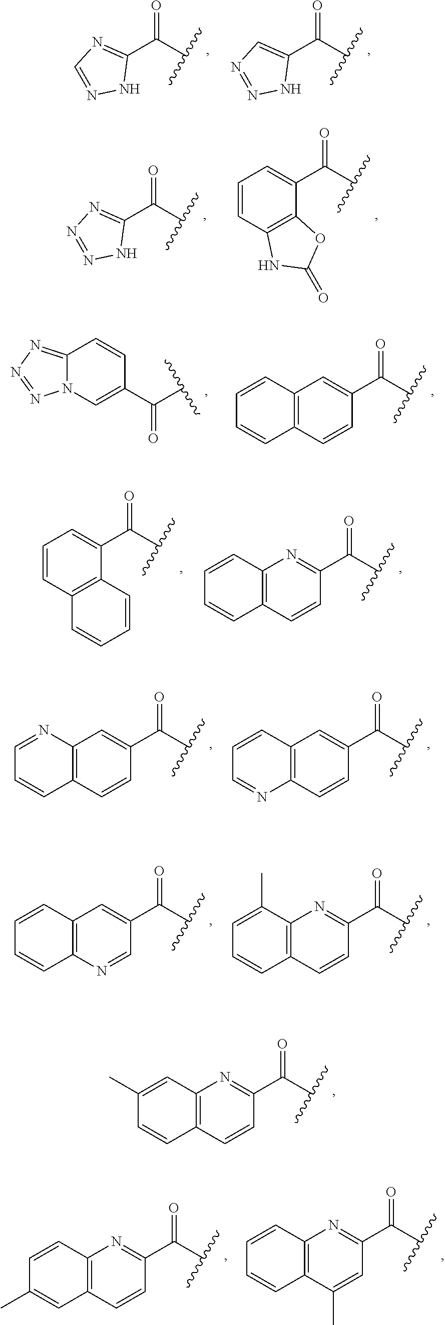

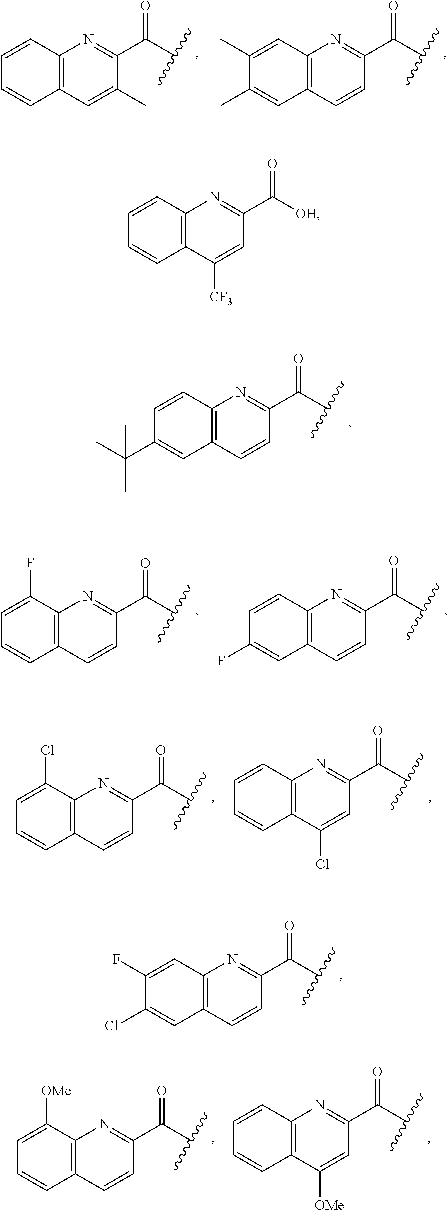

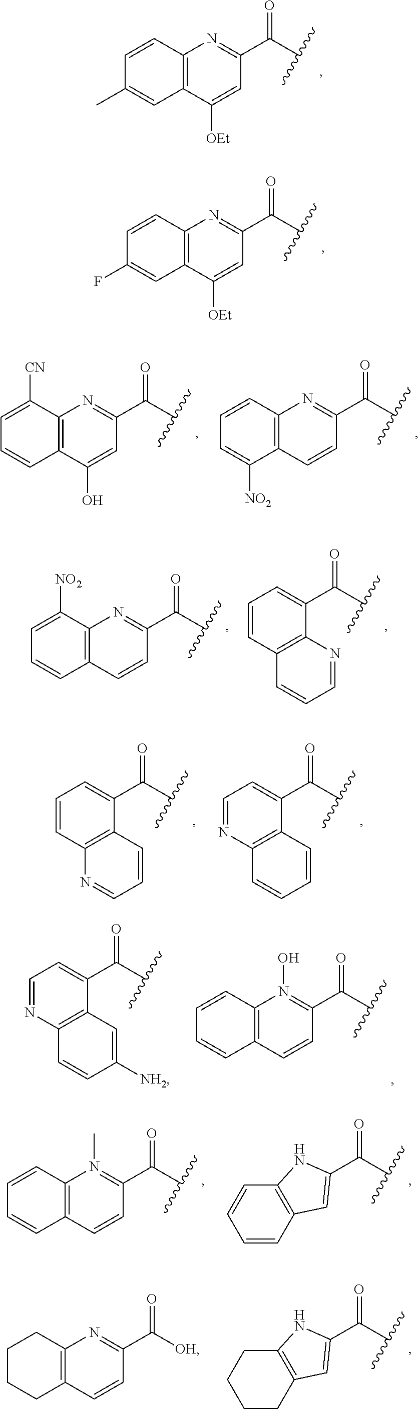

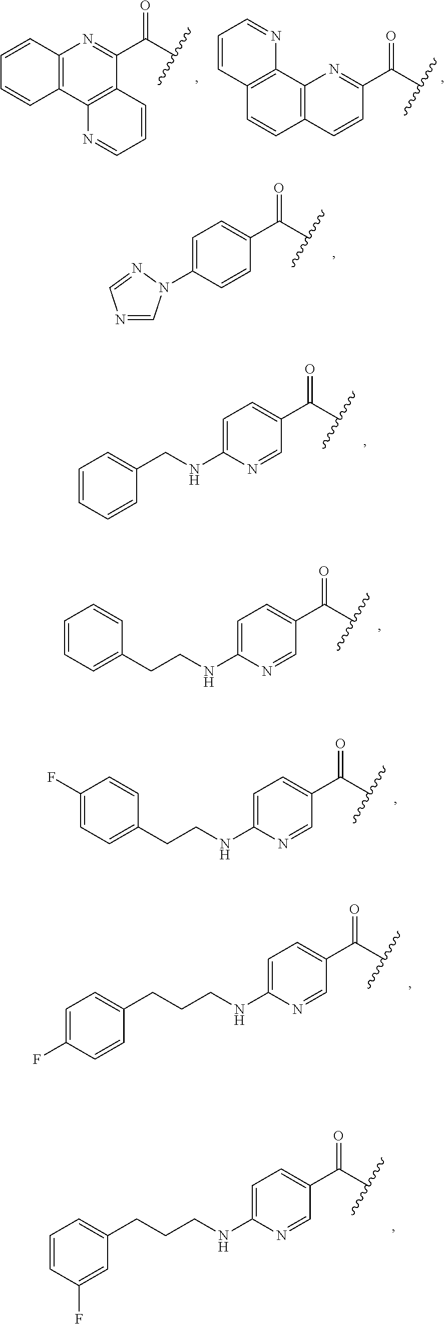

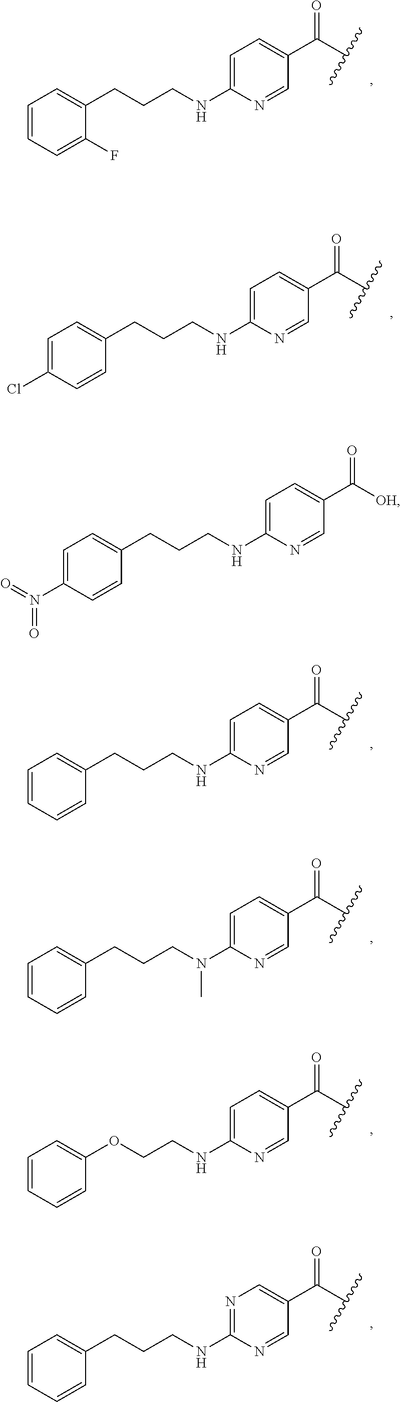

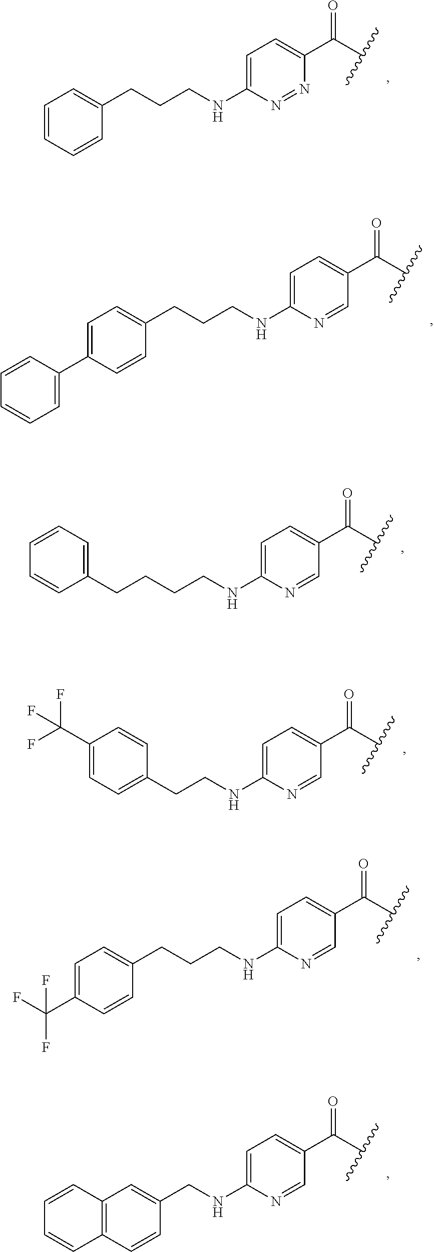 [SMILES: *C(=O)c1ccc(NCCCc2ccccc2)nn1 ; *C(=O)c1ccc(NCCCc2ccc(-c3ccccc3)cc2)nc1 ; *C(=O)c1ccc(NCCCCc2ccccc2)nc1 ; *C(=O)c1ccc(NCCc2ccc(C(F)(F)F)cc2)nc1 ; *C(=O)c1ccc(NCCCc2ccc(C(F)(F)F)cc2)nc1 ; *C(=O)c1ccc(NCc2ccc3ccccc3c2)nc1]

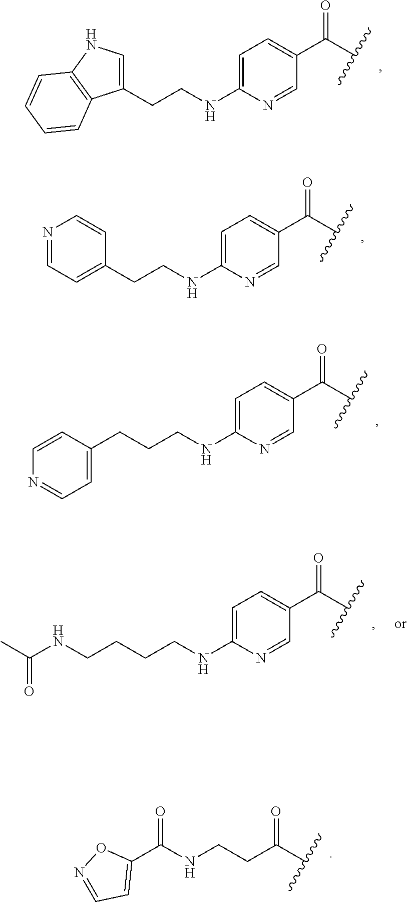 [SMILES: *C(=O)c1ccc(NCCc2c[nH]c3ccccc23)nc1 ; *C(=O)c1ccc(NCCc2ccncc2)nc1 ; *C(=O)c1ccc(NCCCc2ccncc2)nc1 ; *C(=O)c1ccc(NCCCCNC(C)=O)nc1 ; *C(=O)CCNC(=O)c1ccno1]

Embodiment 133. The compound of any one of embodiments 89-126, or a pharmaceutically acceptable salt thereof, wherein R^{a} is

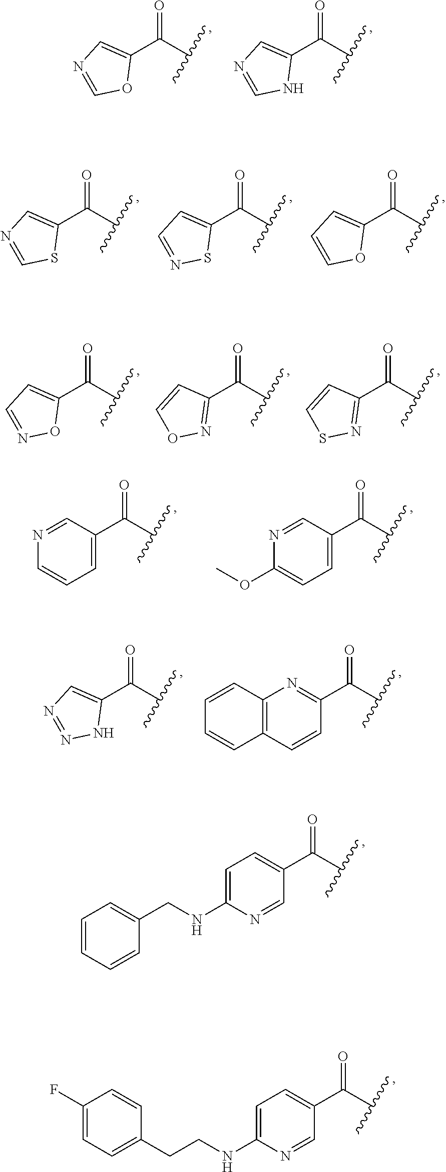

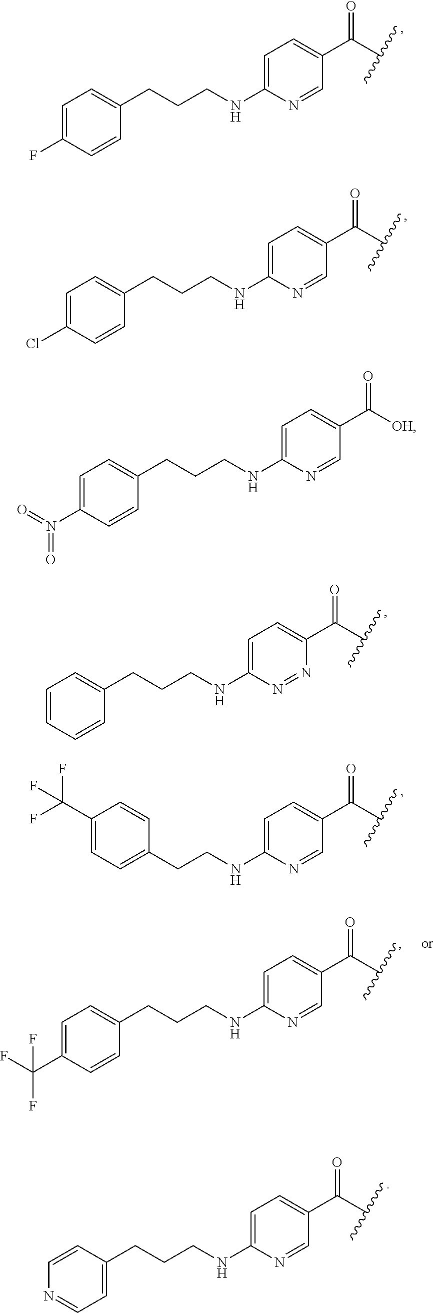

Embodiment 134. The compound of any one of embodiments 89-126, or a pharmaceutically acceptable salt thereof, wherein R^{a} is

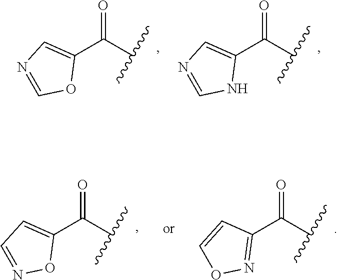 [SMILES: *C(=O)c1cnco1 ; *C(=O)c1cnc[nH]1 ; *C(=O)c1ccno1 ; *C(=O)c1ccon1]

Embodiment 135. The compound of any one of embodiments 89-134, or a pharmaceutically acceptable salt thereof, wherein -L⁵-, -L⁶-, or -L⁷- are each independently selected from the group consisting of: —NR¹⁵—C₁-C₆ alkyl-C(═O)—, —NR¹⁵—(CH₂CH₂O)—C₁-C₆ alkyl-C(═O)—, —NR¹⁵(CH₂CH₂NR¹⁶)_{y}—C₁-C₆ alkyl-C(═O)—, —NR¹⁵—C₁-C₆ alkyl-(substituted or unsubstituted heterocycloalkyl)-C(═O)—, —NR¹⁵-(substituted or unsubstituted heterocycloalkyl)-C₁-C₆ alkyl-C(═O)—, —NR¹⁵—C₁-C₆ alkyl-(substituted or unsubstituted cyclohexyl)-C(═O)—, and —NR¹⁵-(substituted or unsubstituted cyclohexyl)-C₁-C₆ alkyl-C(═O)—;
R¹⁵ and R¹⁶ are each independently selected from H or —C₁-C₄ alkyl; and
y is 1, 2, 3, 4, 5, or 6.

Embodiment 136. The compound of any one of embodiments 89-134, or a pharmaceutically acceptable salt thereof, wherein -L⁵-, -L⁶-, or -L⁷- are each independently selected from the group consisting of:

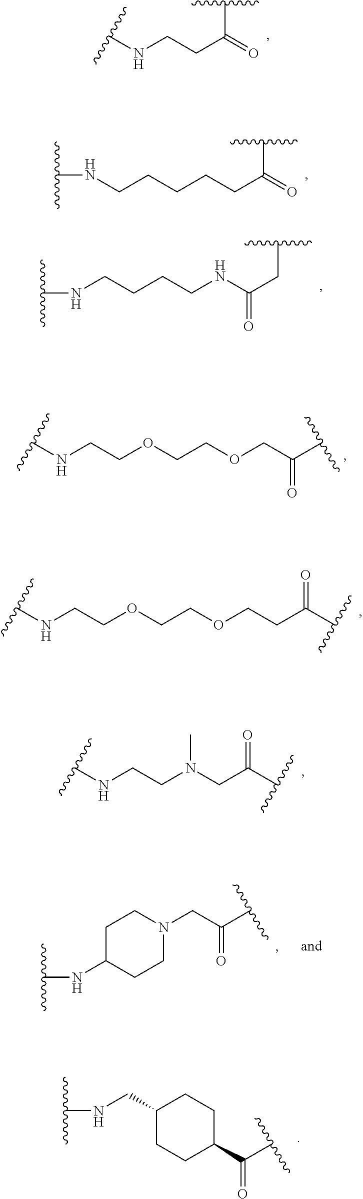

Embodiment 138. The compound of embodiment 89, or a pharmaceutically acceptable salt thereof, wherein the compound of Formula (VII) has the following chemical structure, or a pharmaceutically acceptable salt thereof:

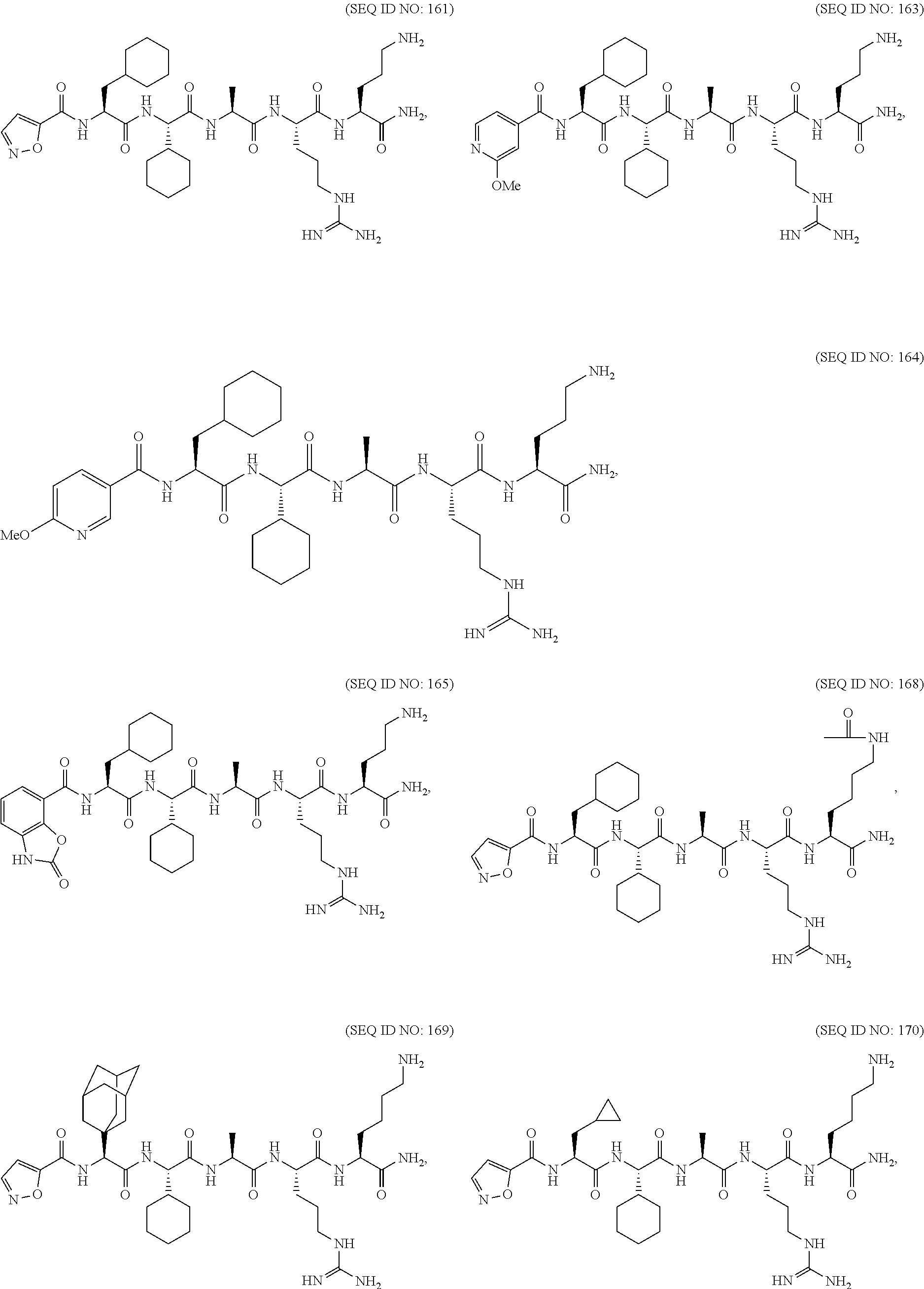

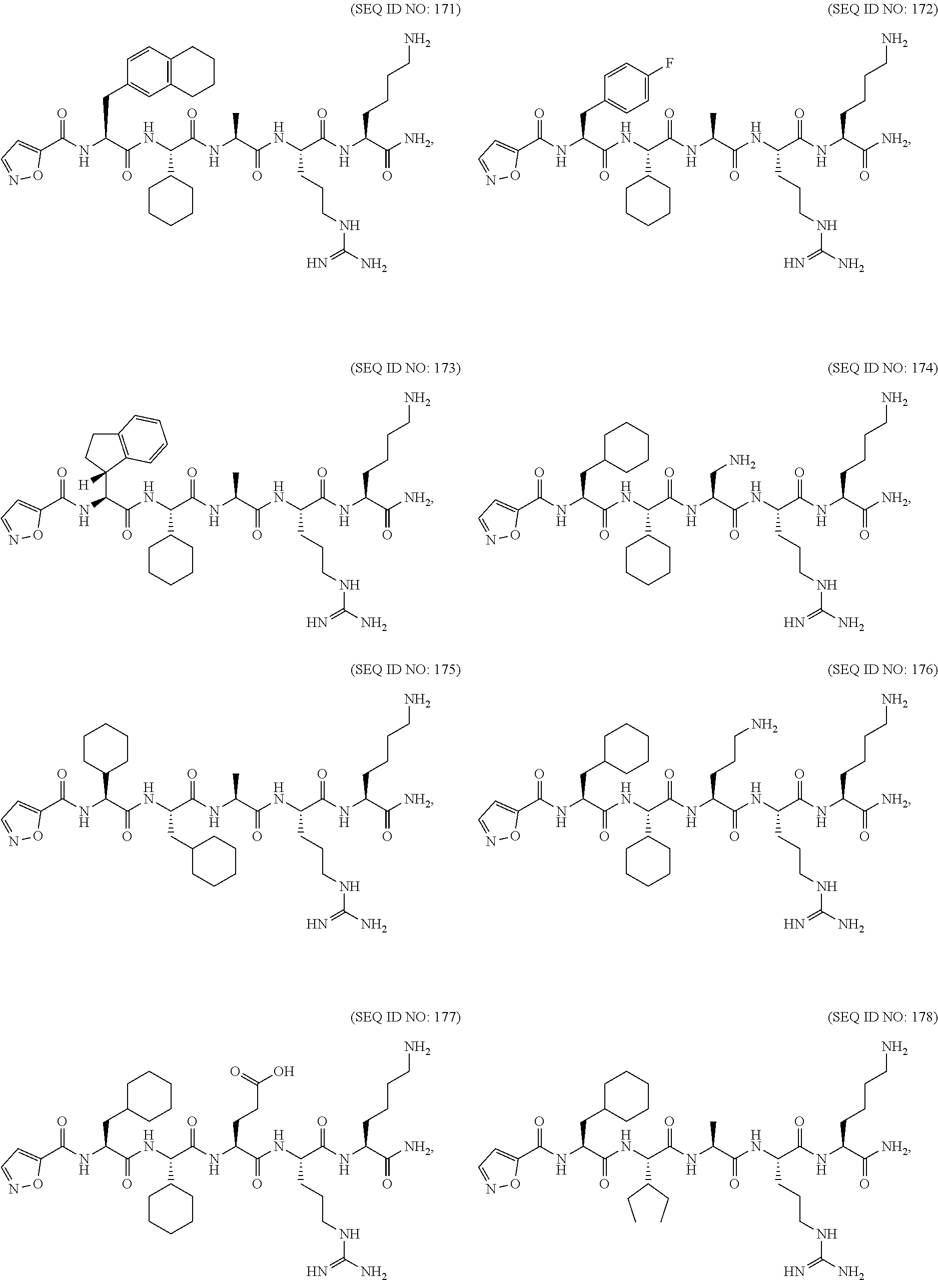

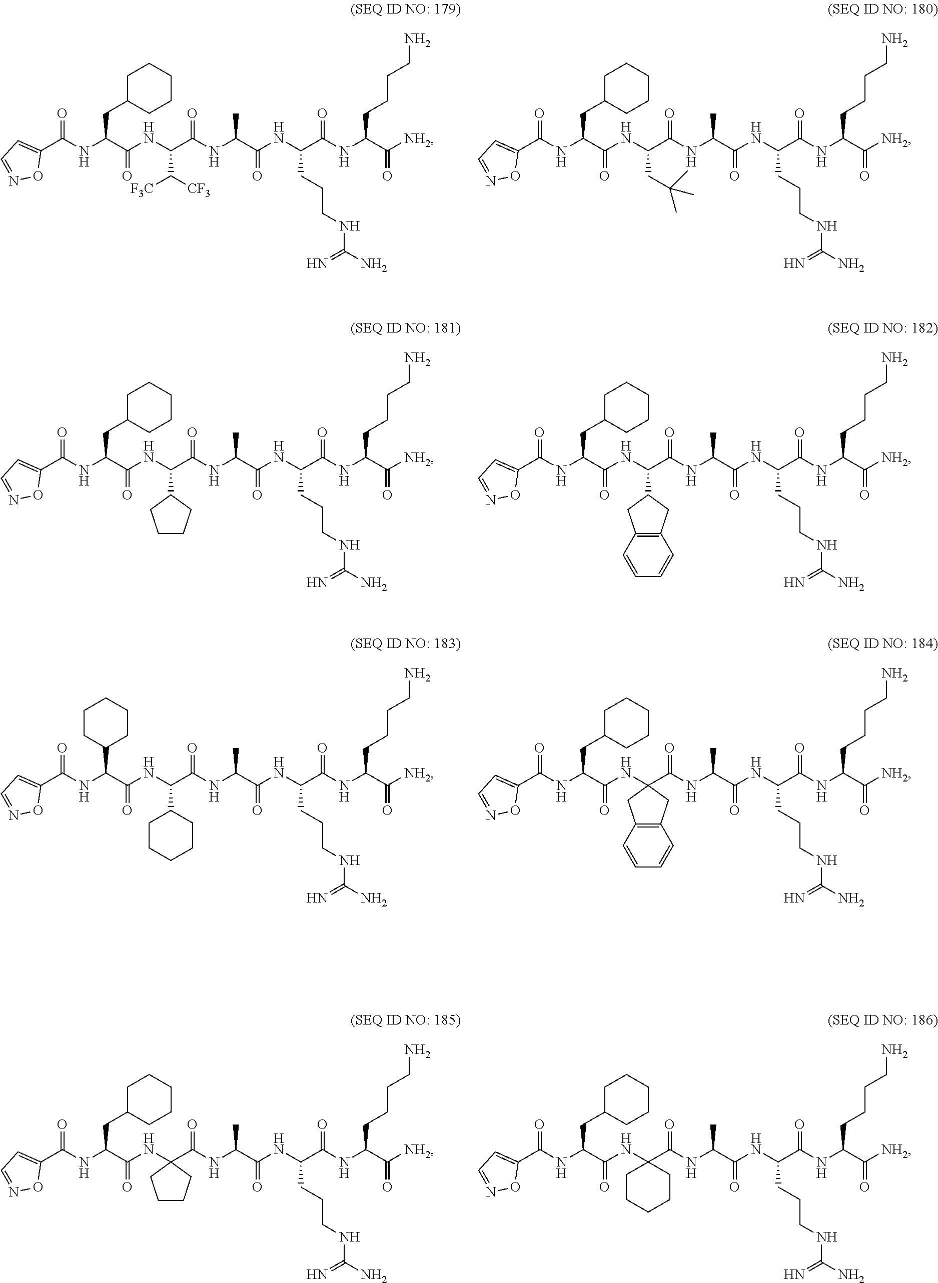

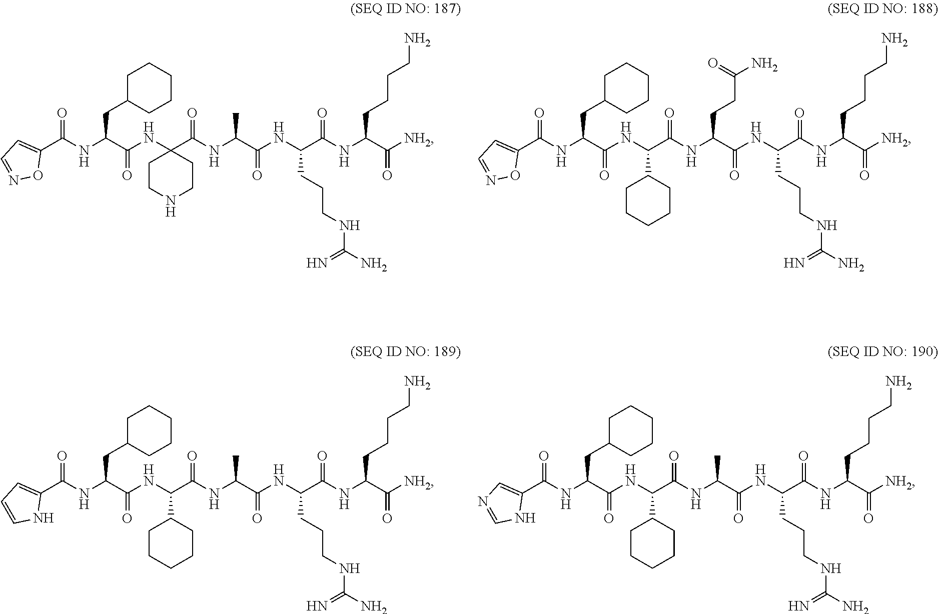 [SMILES: C[C@H](NC(=O)C1(NC(=O)[C@H](CC2CCCCC2)NC(=O)c2ccno2)CCNCC1)C(=O)N[C@@H](CCCNC(=N)N)C(=O)N[C@@H](CCCCN)C(N)=O ; N=C(N)NCCC[C@H](NC(=O)[C@H](CCC(N)=O)NC(=O)[C@@H](NC(=O)[C@H](CC1CCCCC1)NC(=O)c1ccno1)C1CCCCC1)C(=O)N[C@@H](CCCCN)C(N)=O ; C[C@H](NC(=O)[C@@H](NC(=O)[C@H](CC1CCCCC1)NC(=O)c1ccc[nH]1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1cnc[nH]1)C1CCCCC1)C(=O)N[C@@H](CCCNC(=N)N)C(=O)N[C@@H](CCCCN)C(N)=O]

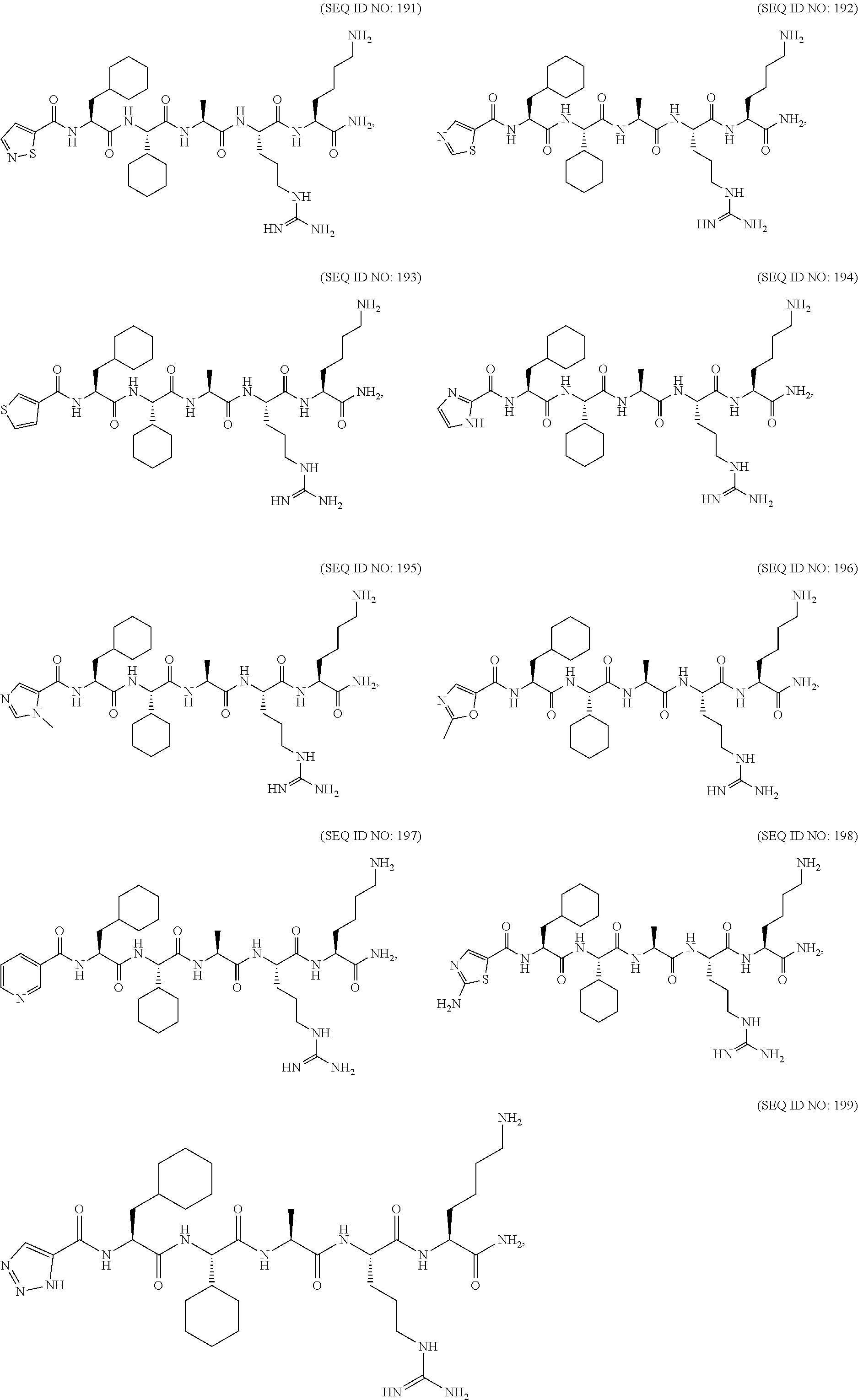

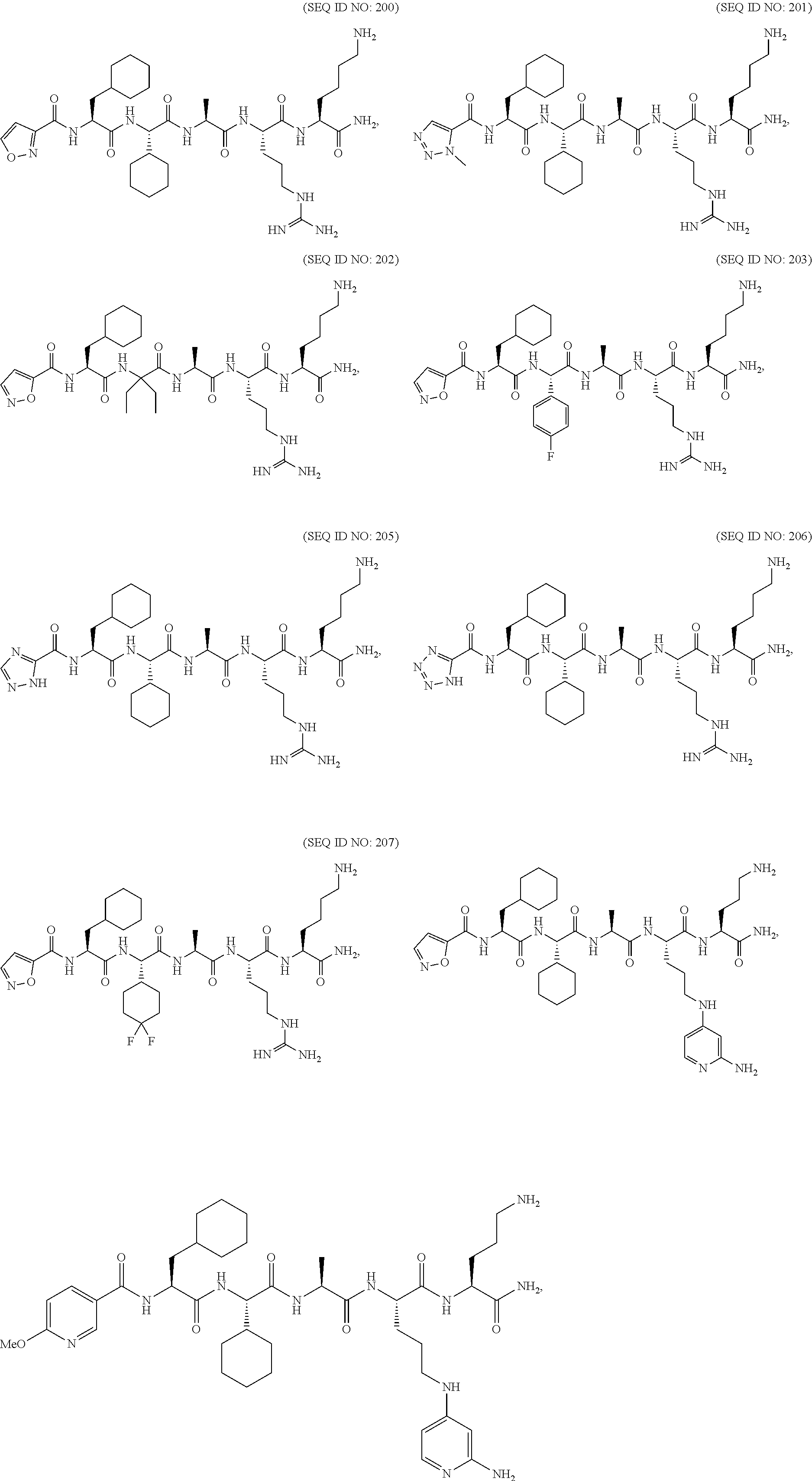

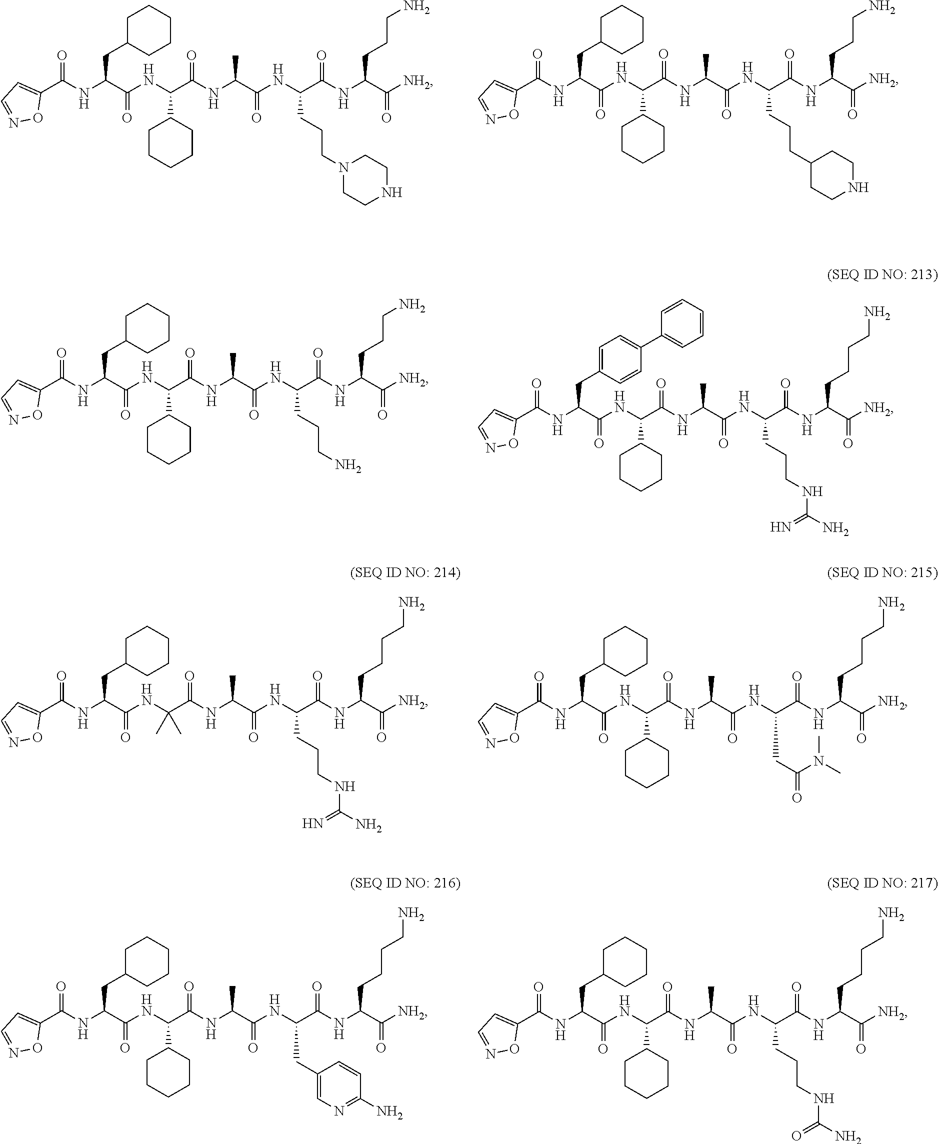

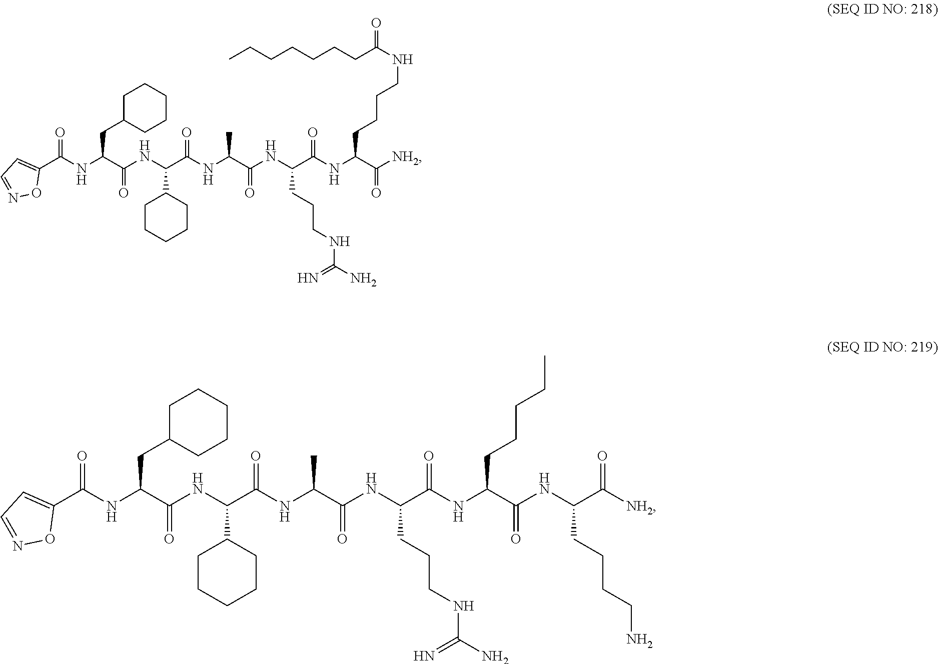 [SMILES: CCCCCCCC(=O)NCCCC[C@H](NC(=O)[C@H](CCCNC(=N)N)NC(=O)[C@H](C)NC(=O)[C@@H](NC(=O)[C@H](CC1CCCCC1)NC(=O)c1ccno1)C1CCCCC1)C(N)=O ; CCCCC[C@H](NC(=O)[C@H](CCCNC(=N)N)NC(=O)[C@H](C)NC(=O)[C@@H](NC(=O)[C@H](CC1CCCCC1)NC(=O)c1ccno1)C1CCCCC1)C(=O)N[C@@H](CCCCN)C(N)=O]

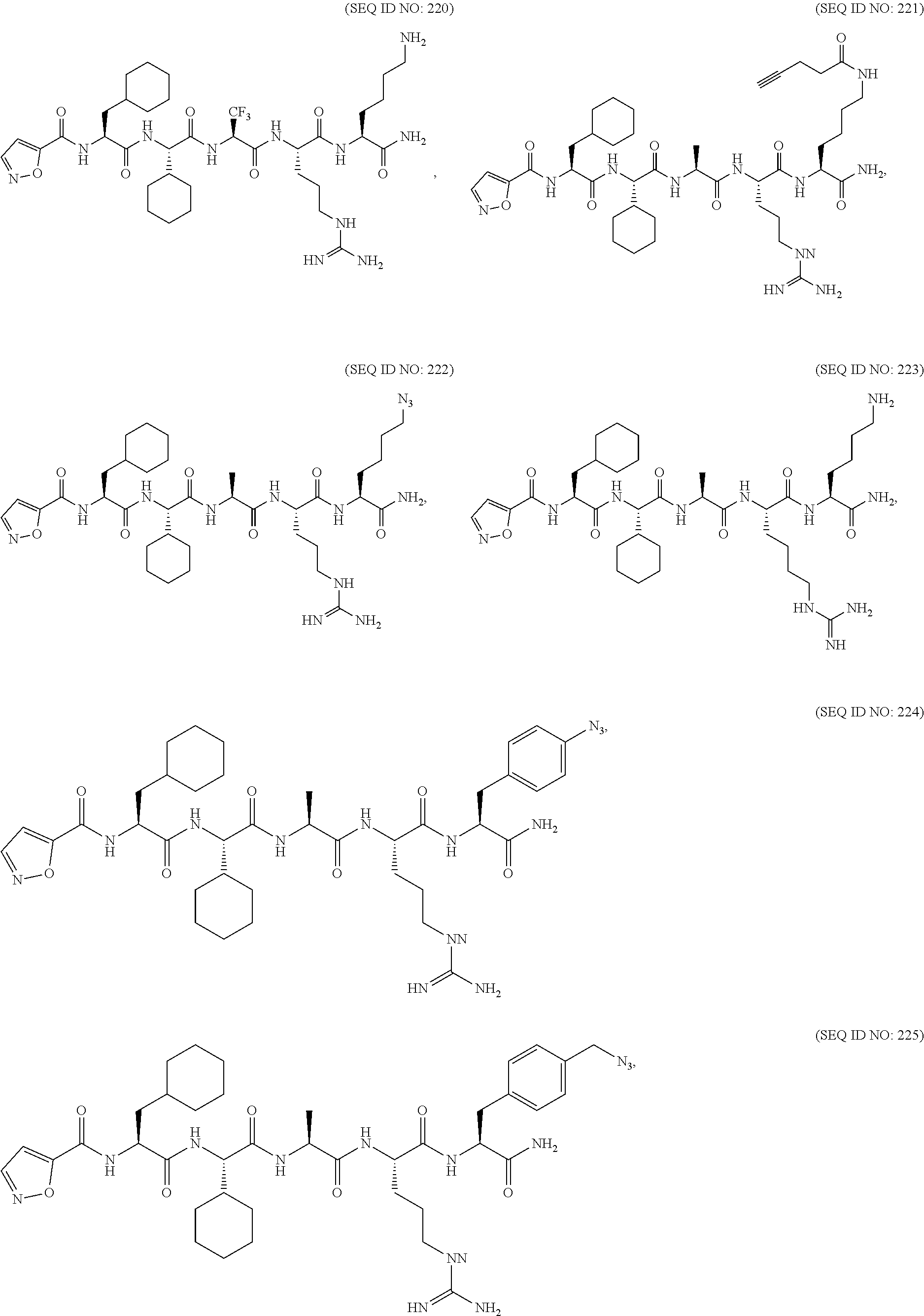

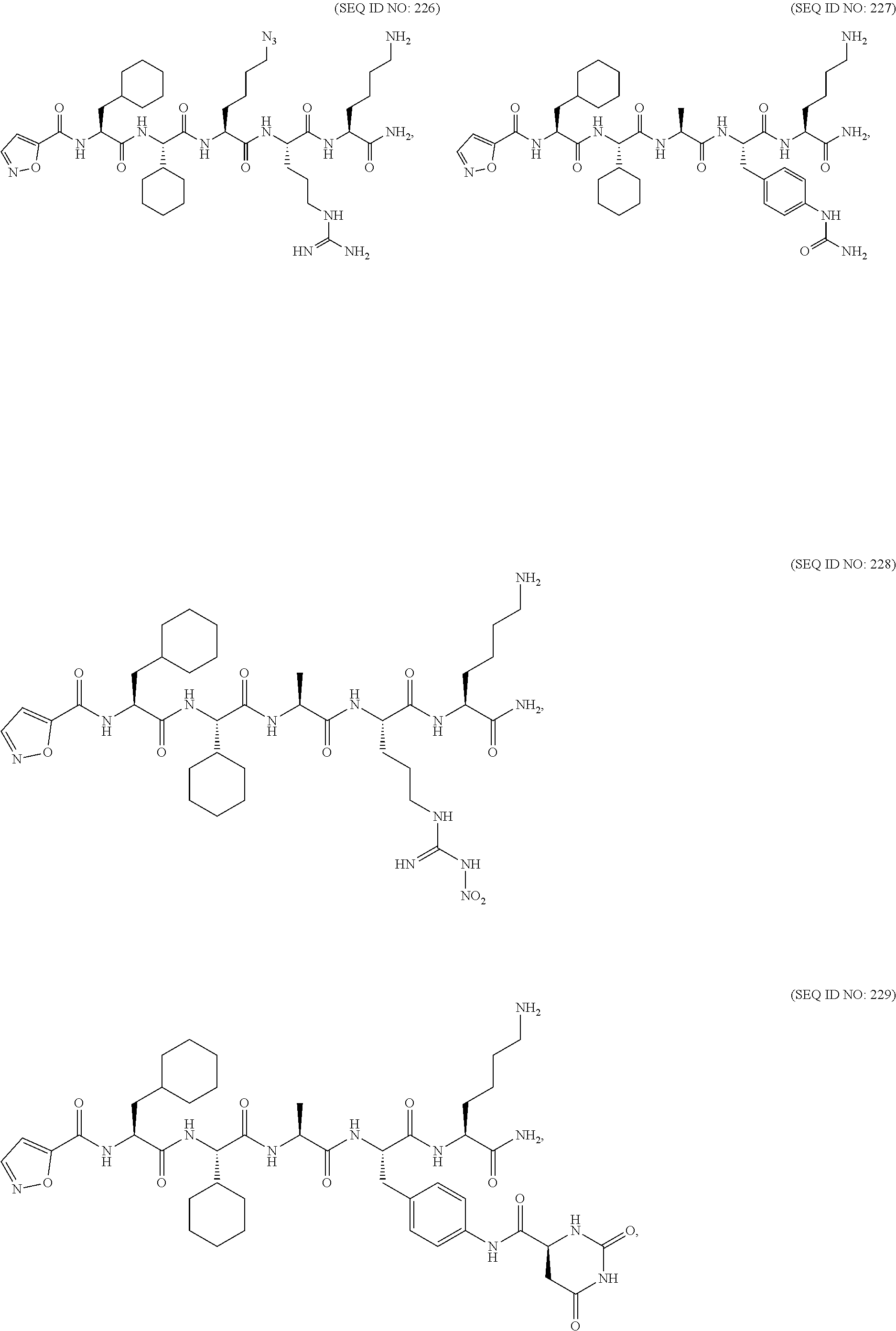

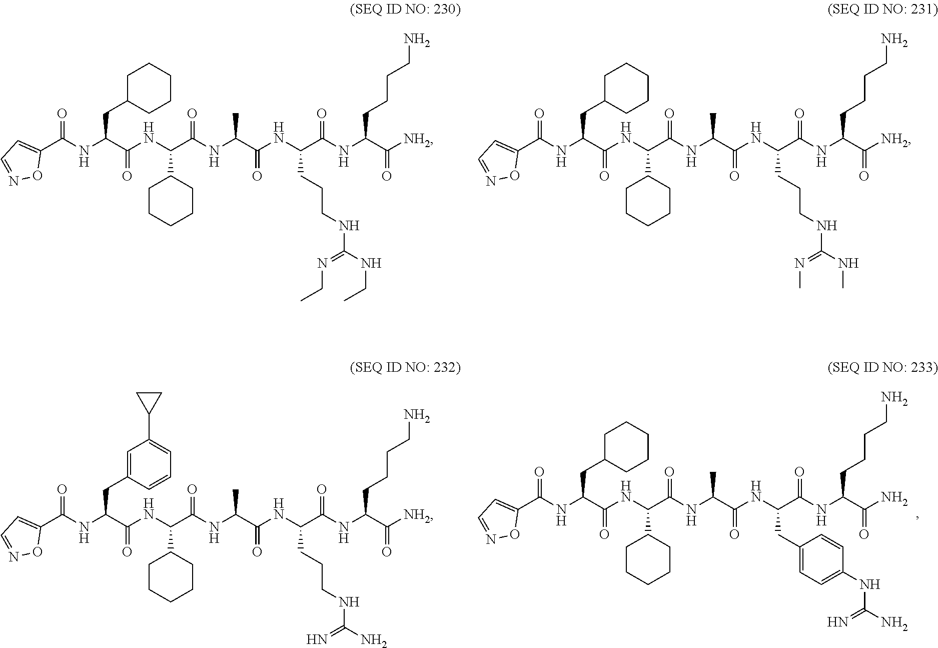 [SMILES: CC/N=C(\NCC)NCCC[C@H](NC(=O)[C@H](C)NC(=O)[C@@H](NC(=O)[C@H](CC1CCCCC1)NC(=O)c1ccno1)C1CCCCC1)C(=O)N[C@@H](CCCCN)C(N)=O ; C/N=C(\NC)NCCC[C@H](NC(=O)[C@H](C)NC(=O)[C@@H](NC(=O)[C@H](CC1CCCCC1)NC(=O)c1ccno1)C1CCCCC1)C(=O)N[C@@H](CCCCN)C(N)=O ; C[C@H](NC(=O)[C@@H](NC(=O)[C@H](Cc1cccc(C2CC2)c1)NC(=O)c1ccno1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccno1)C1CCCCC1)C(=O)N[C@@H](Cc1ccc(NC(=N)N)cc1)C(=O)N[C@@H](CCCCN)C(N)=O]

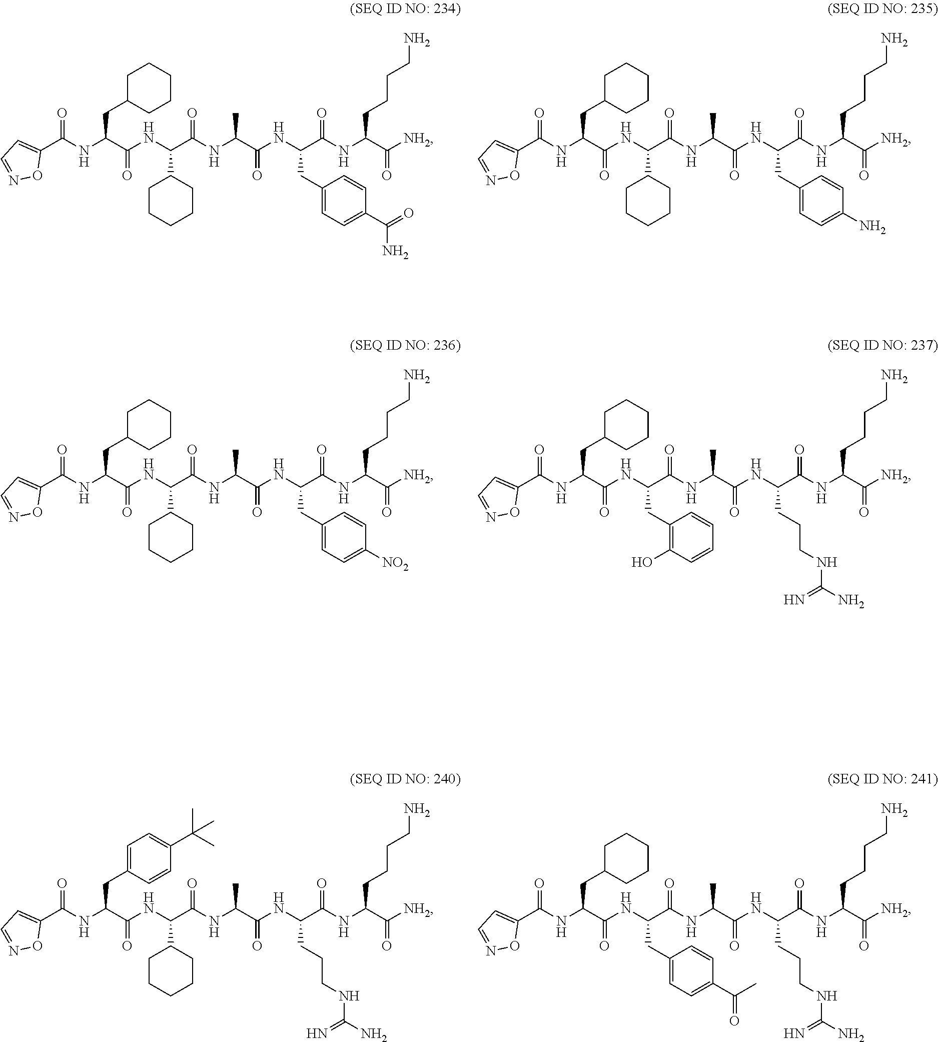

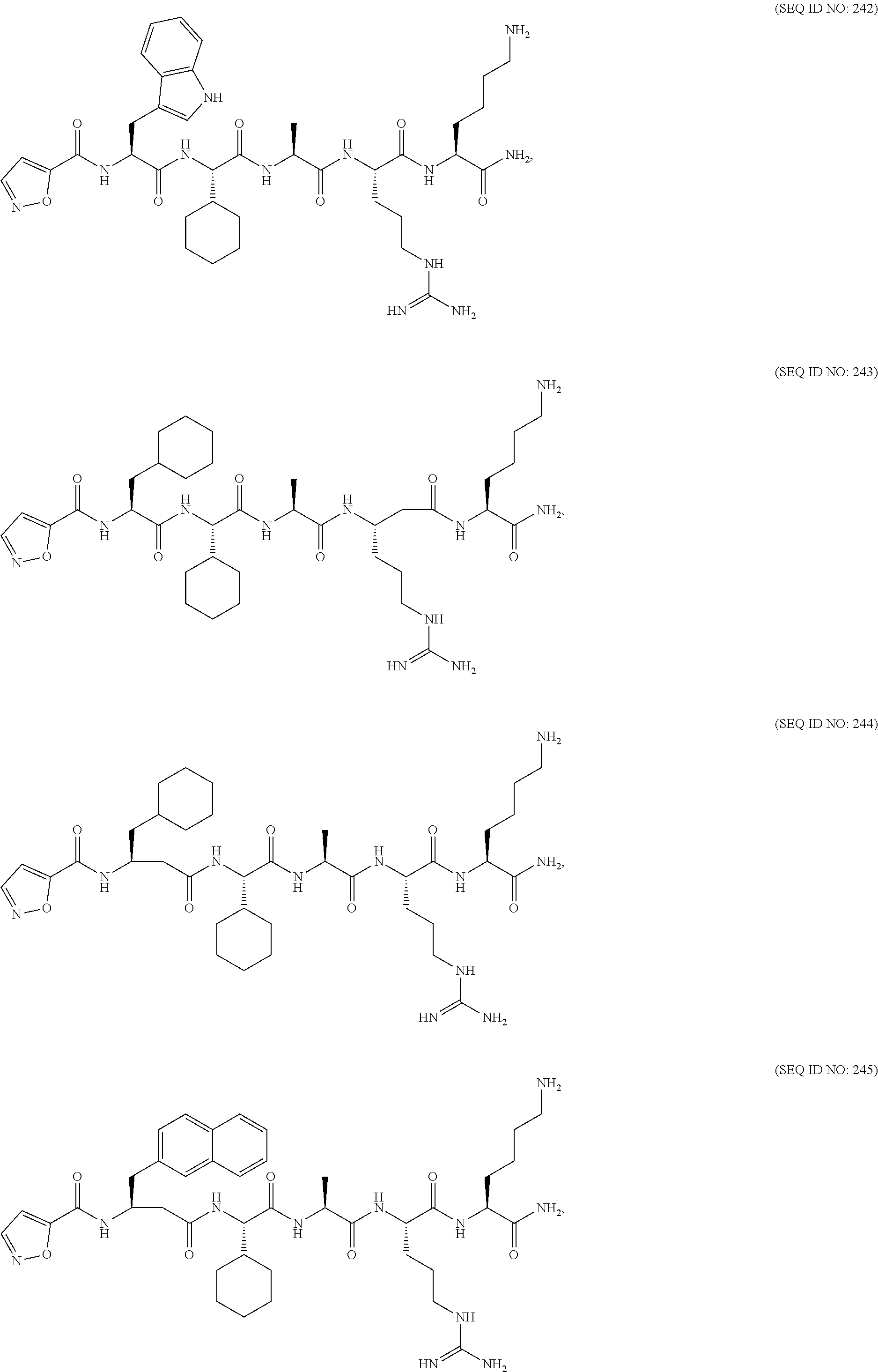 [SMILES: C[C@H](NC(=O)[C@@H](NC(=O)[C@H](Cc1c[nH]c2ccccc12)NC(=O)c1ccno1)C1CCCCC1)C(=O)N[C@@H](CCCNC(=N)N)C(=O)N[C@@H](CCCCN)C(N)=O ; C[C@H](NC(=O)[C@@H](NC(=O)[C@H](CC1CCCCC1)NC(=O)c1ccno1)C1CCCCC1)C(=O)N[C@@H](CCCNC(=N)N)CC(=O)N[C@@H](CCCCN)C(N)=O ; C[C@H](NC(=O)[C@@H](NC(=O)C[C@H](CC1CCCCC1)NC(=O)c1ccno1)C1CCCCC1)C(=O)N[C@@H](CCCNC(=N)N)C(=O)N[C@@H](CCCCN)C(N)=O ; C[C@H](NC(=O)[C@@H](NC(=O)C[C@H](Cc1ccc2ccccc2c1)NC(=O)c1ccno1)C1CCCCC1)C(=O)N[C@@H](CCCNC(=N)N)C(=O)N[C@@H](CCCCN)C(N)=O]

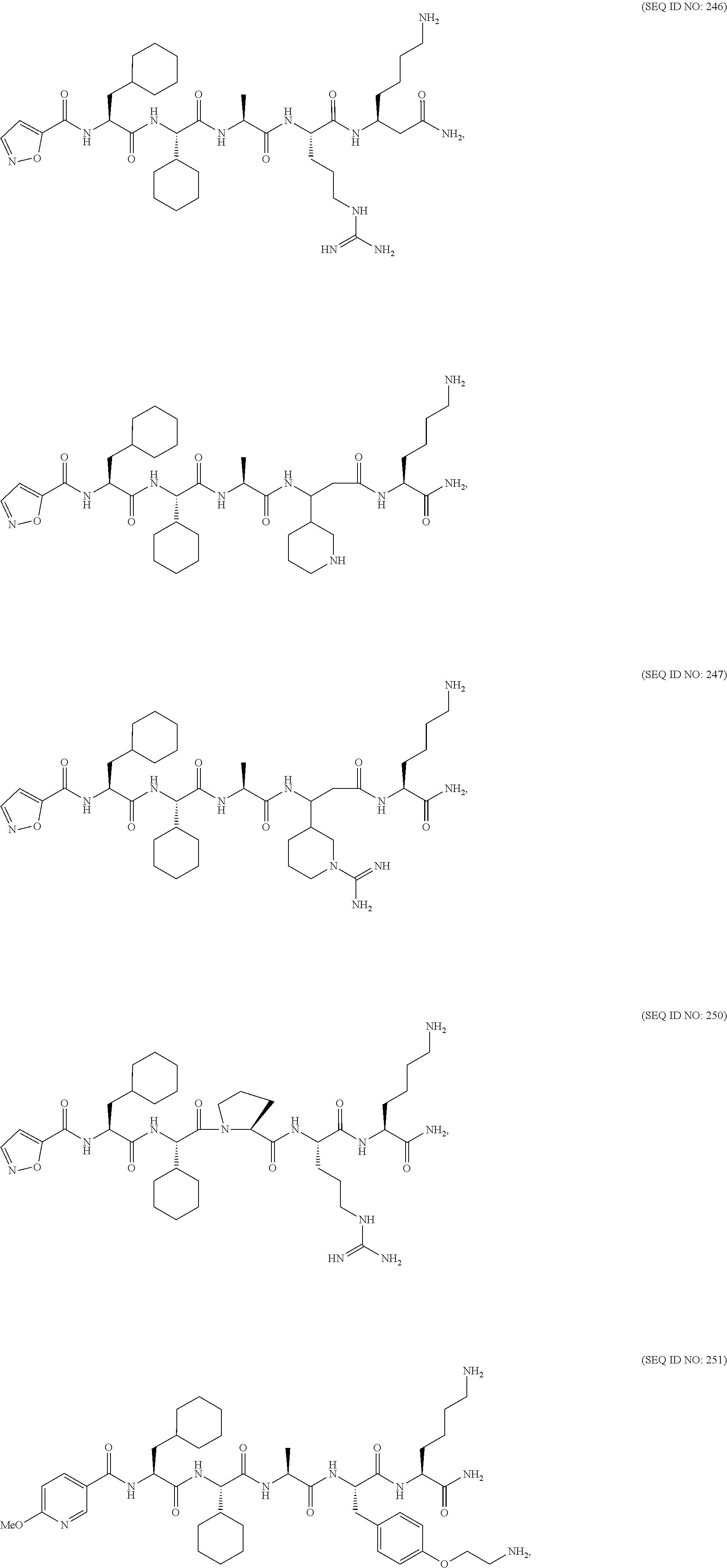

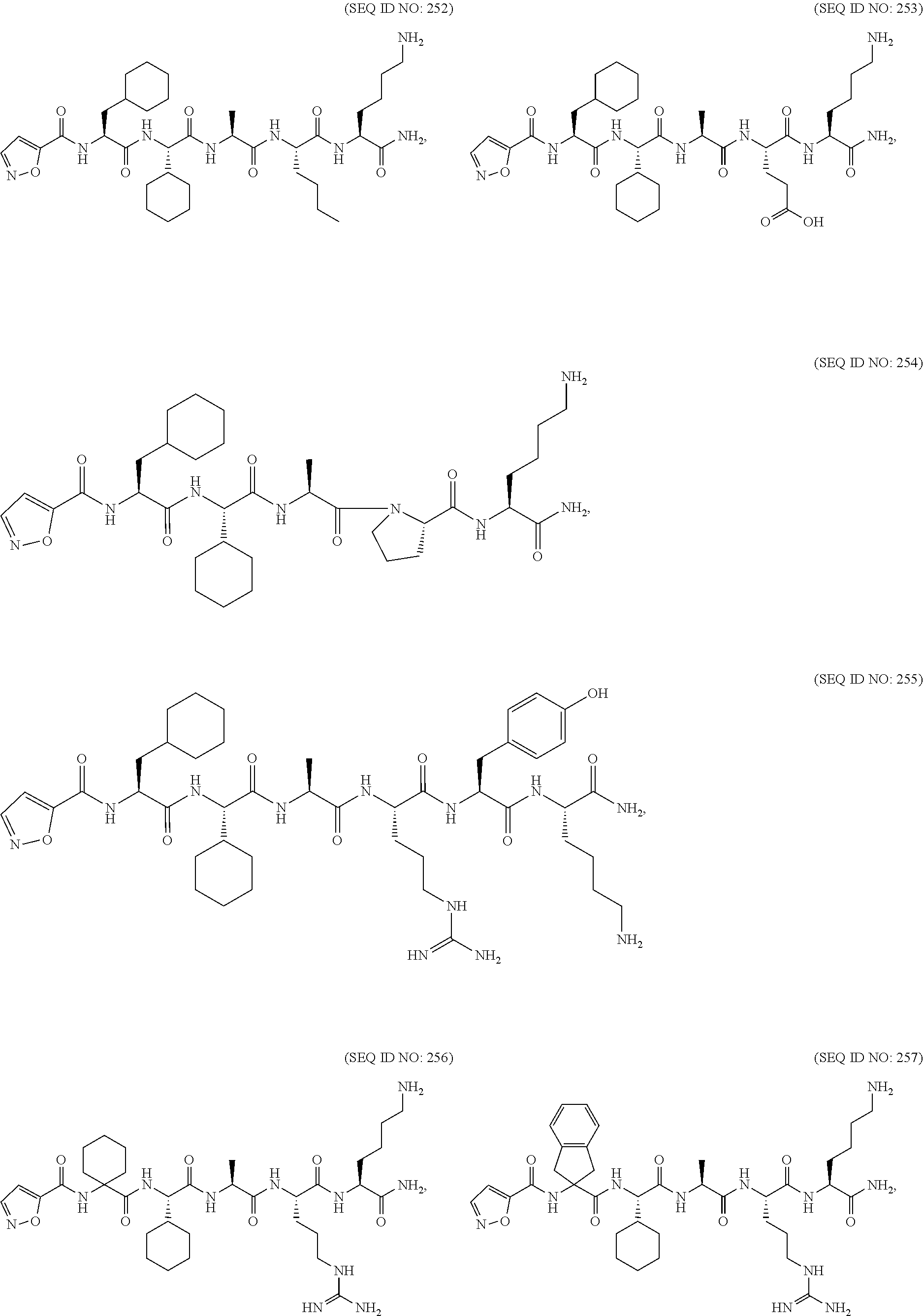

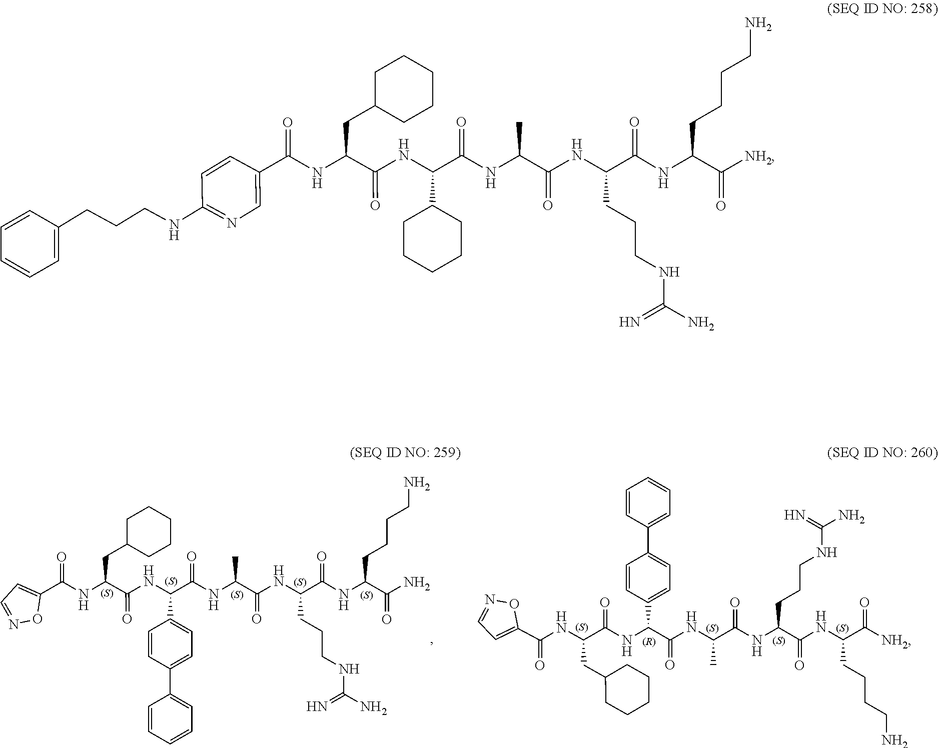

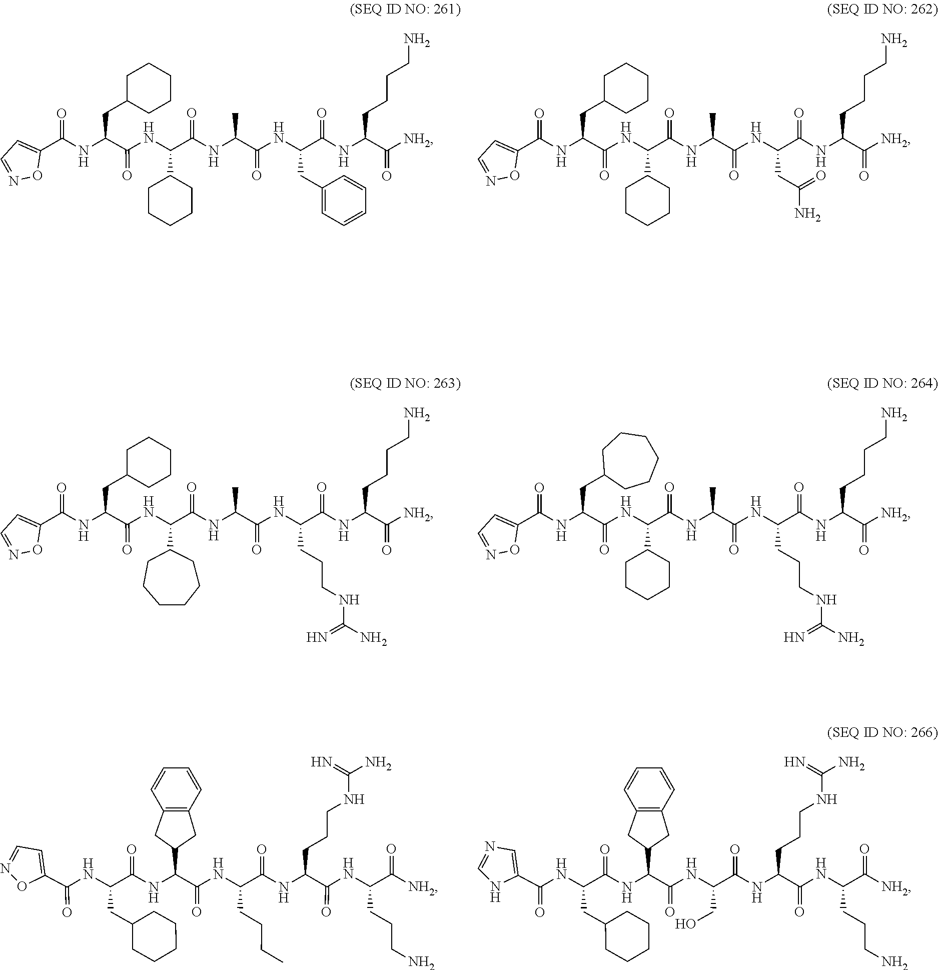

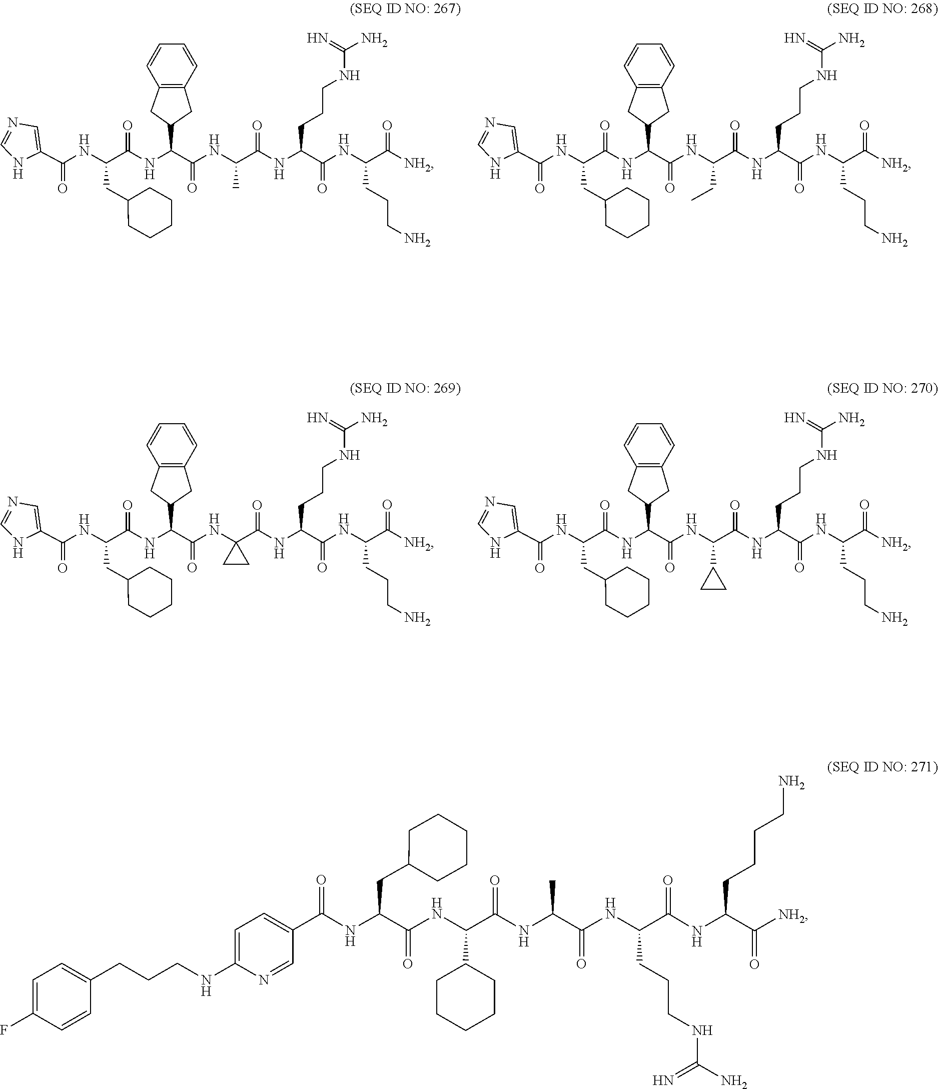

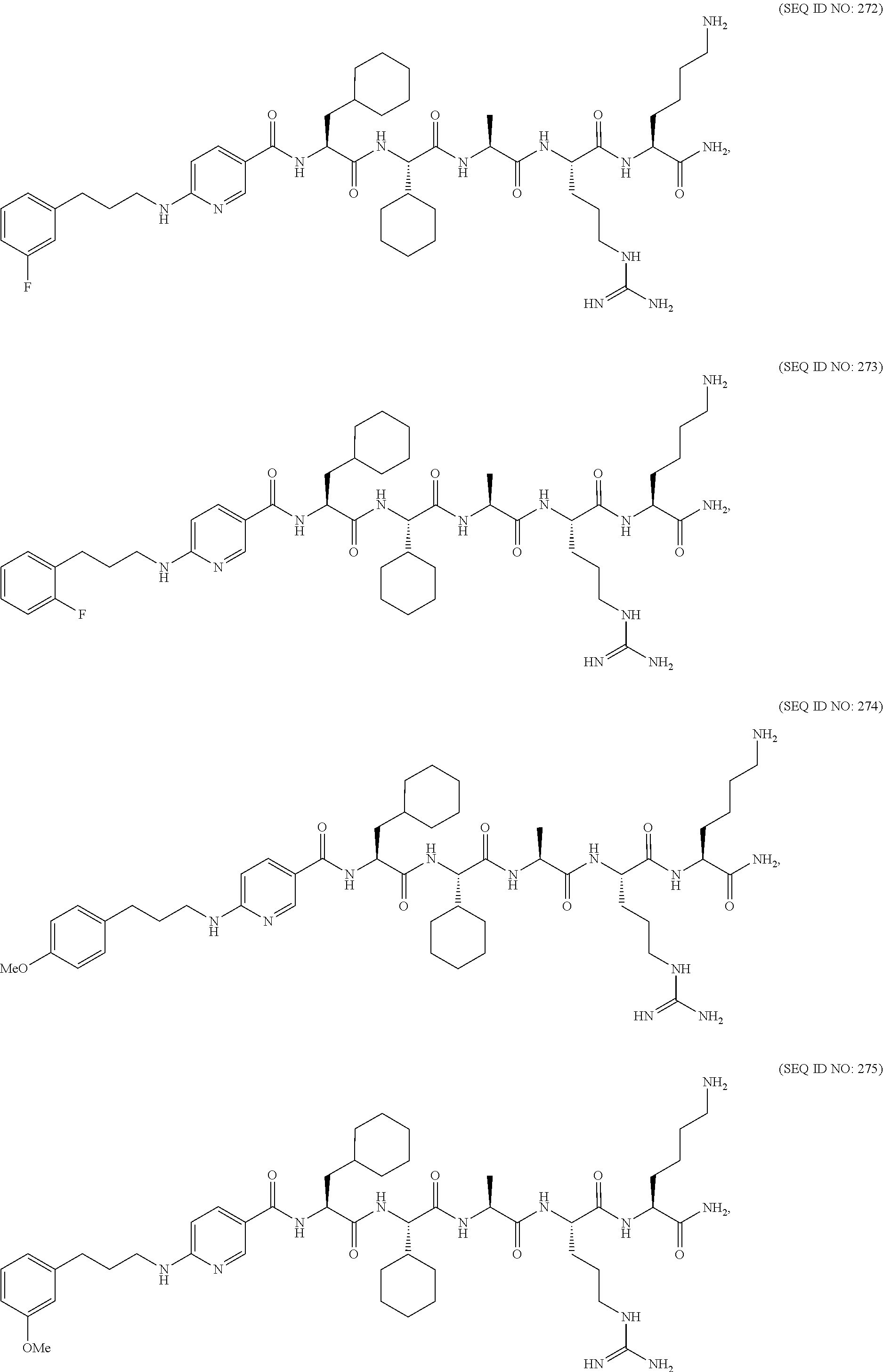

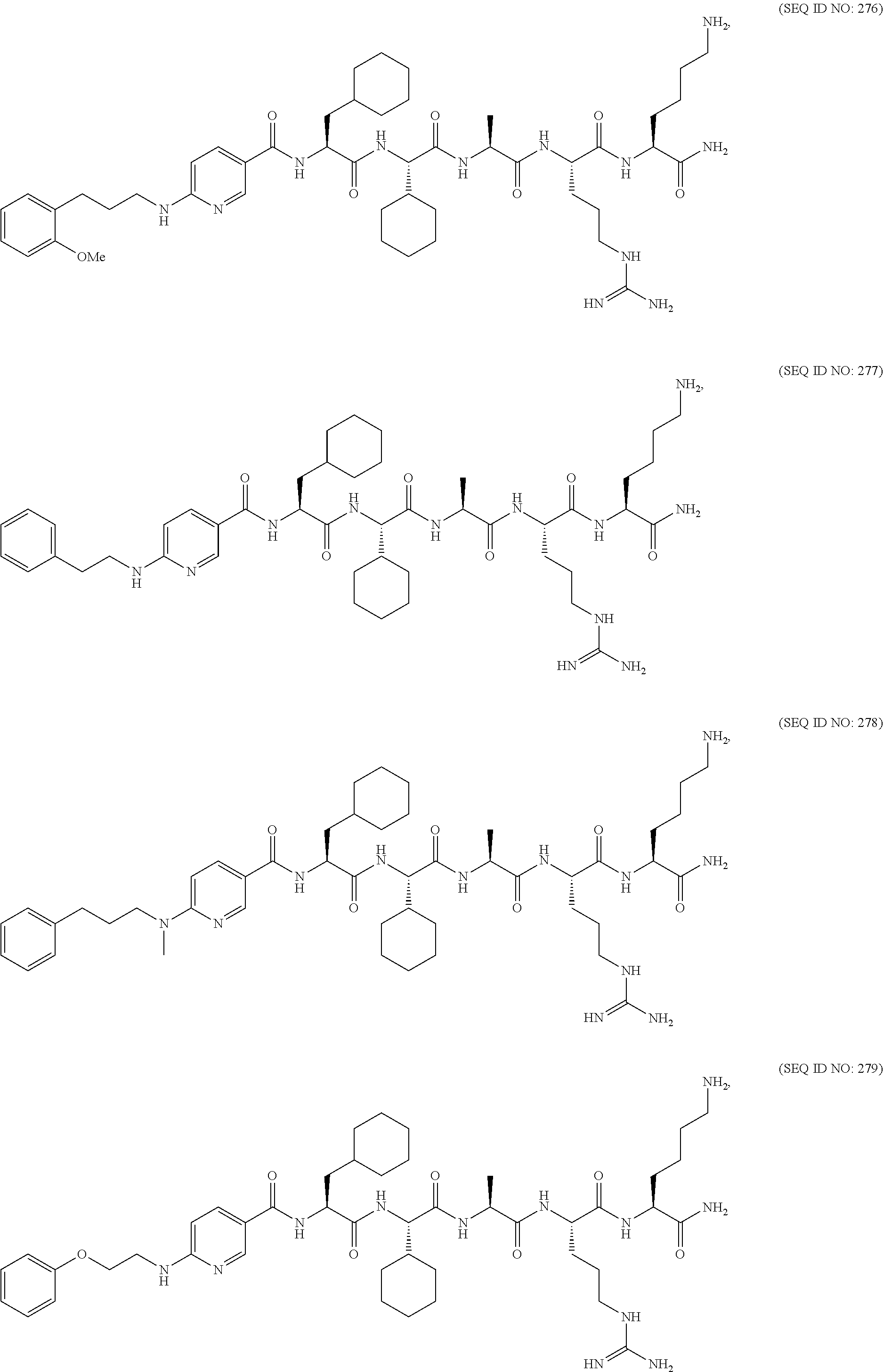

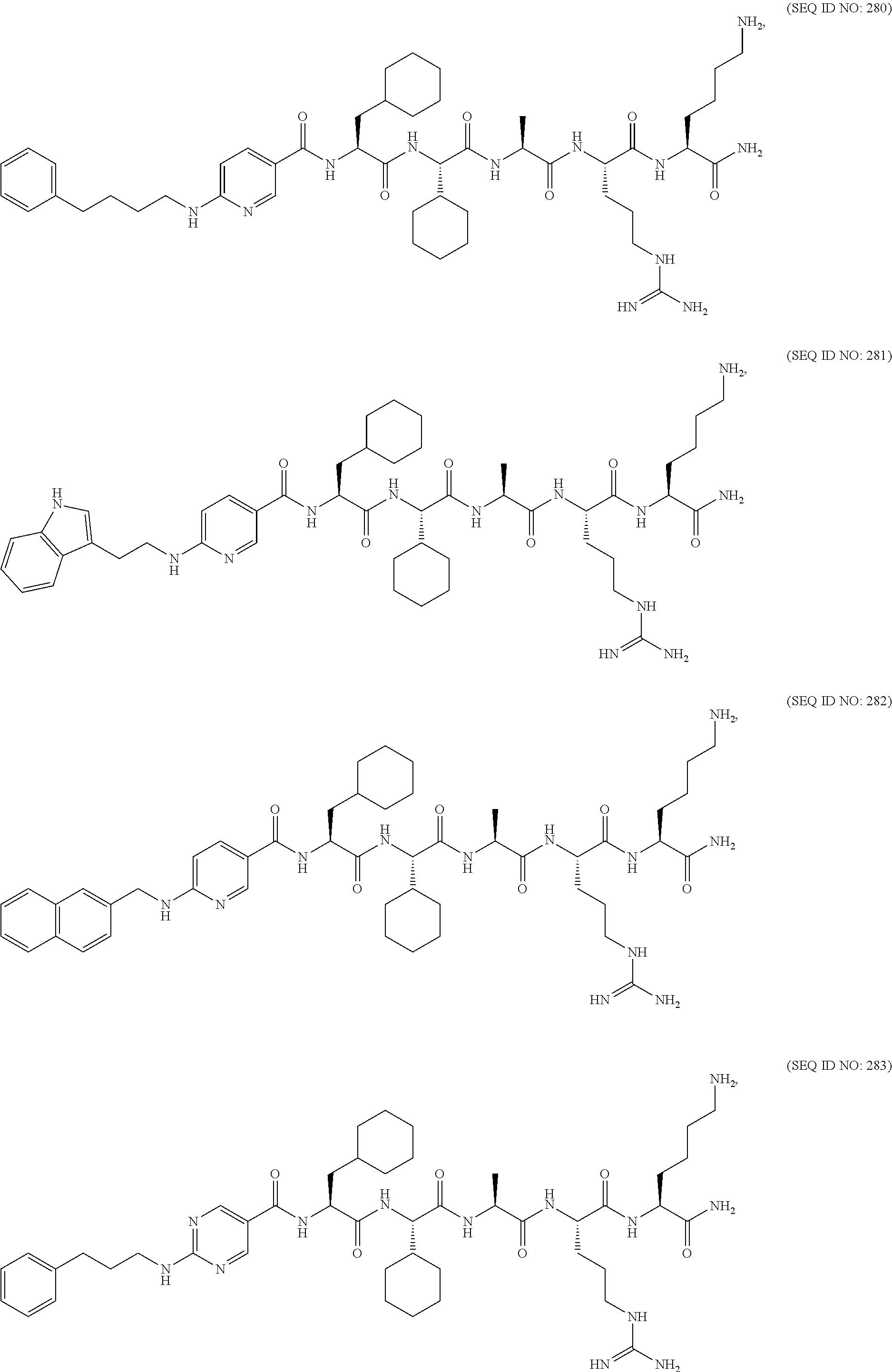

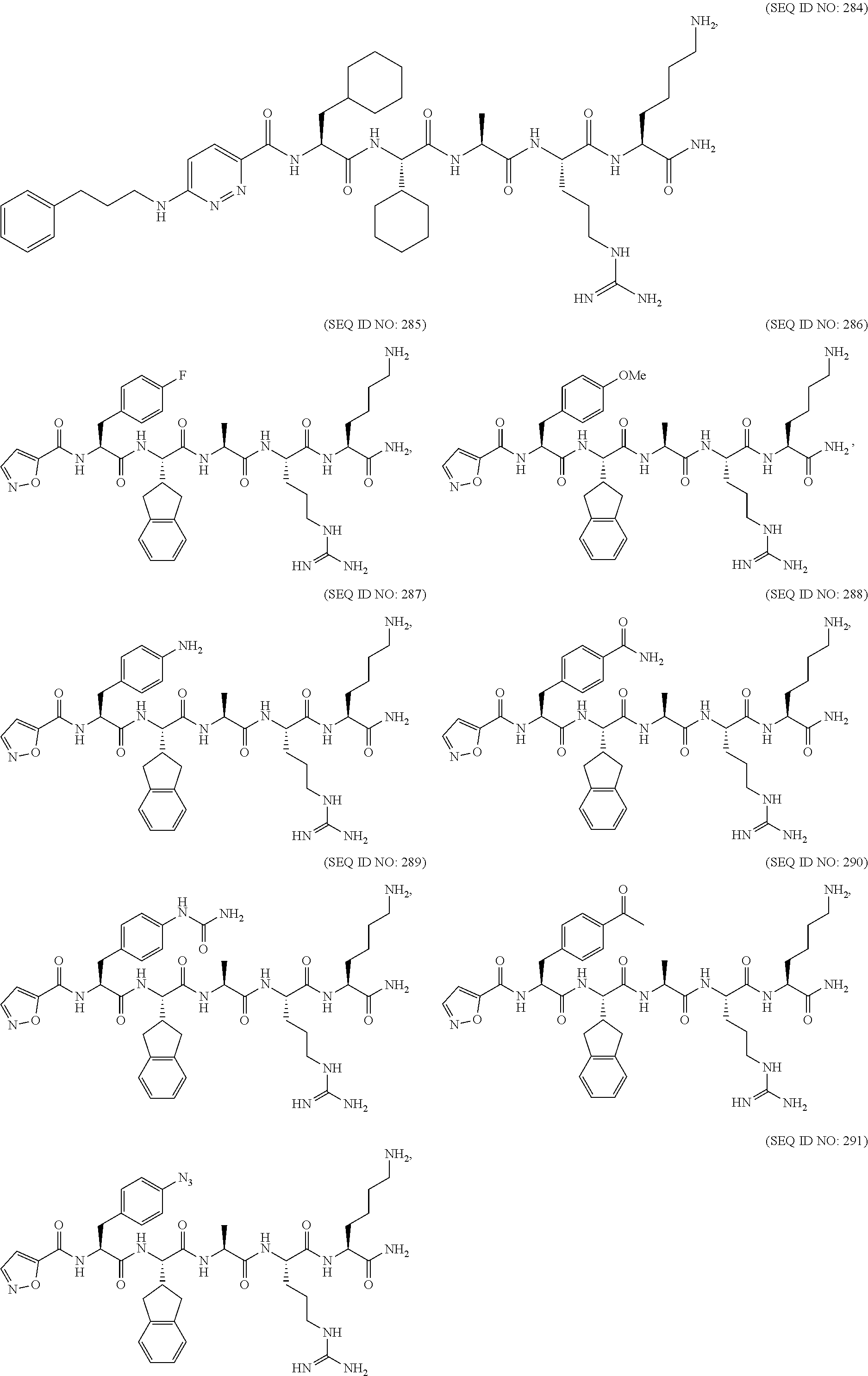

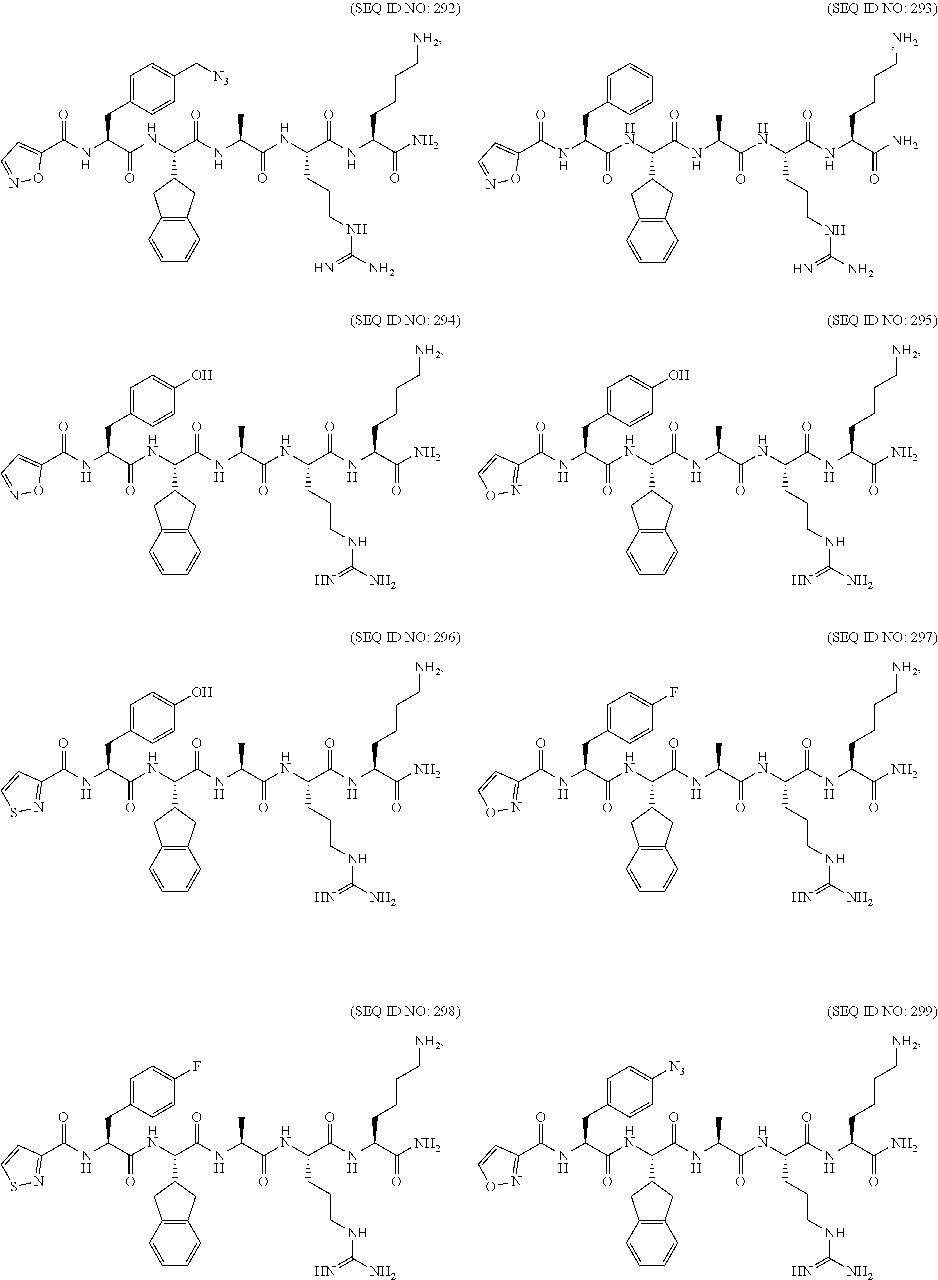

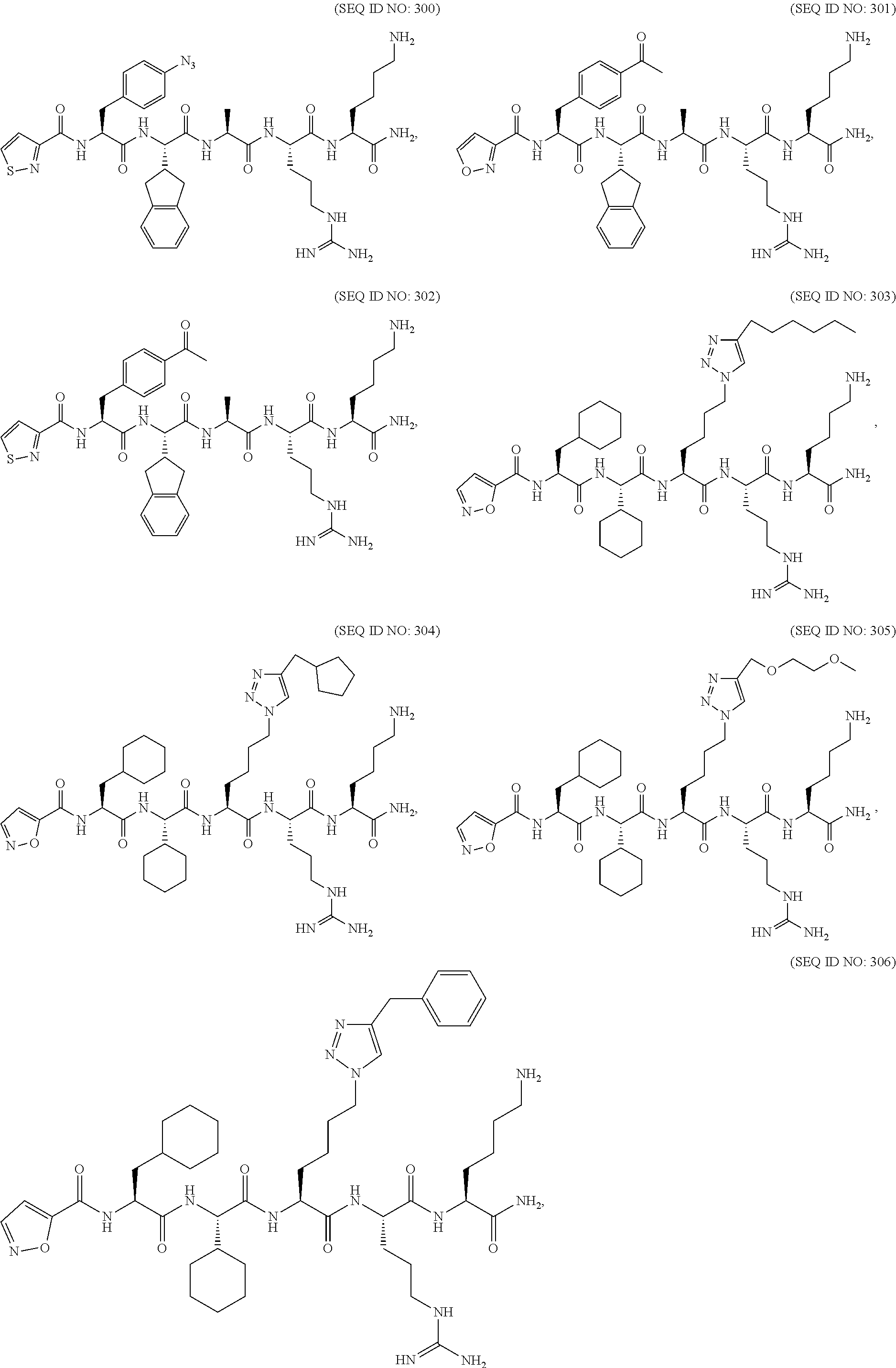

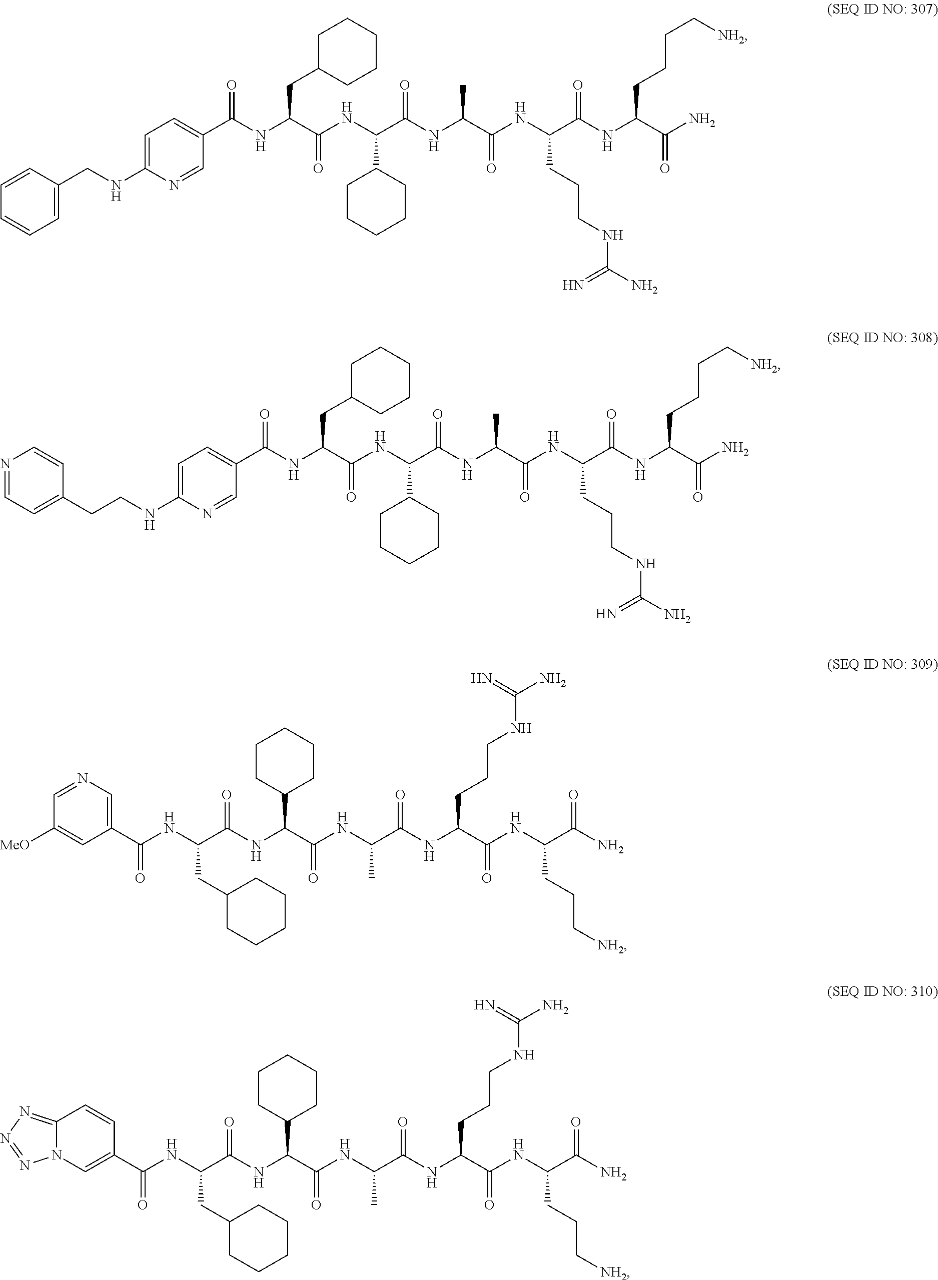

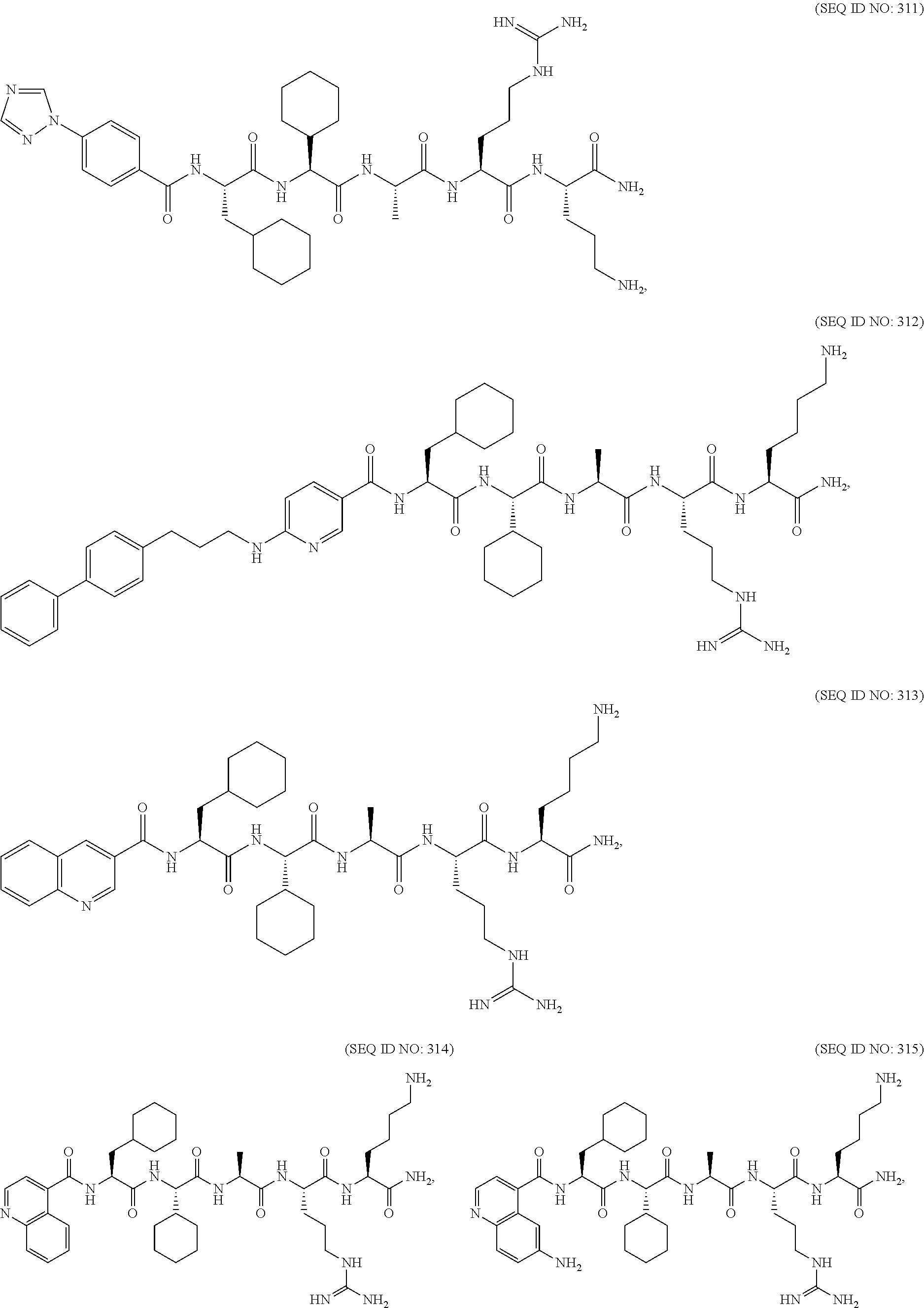

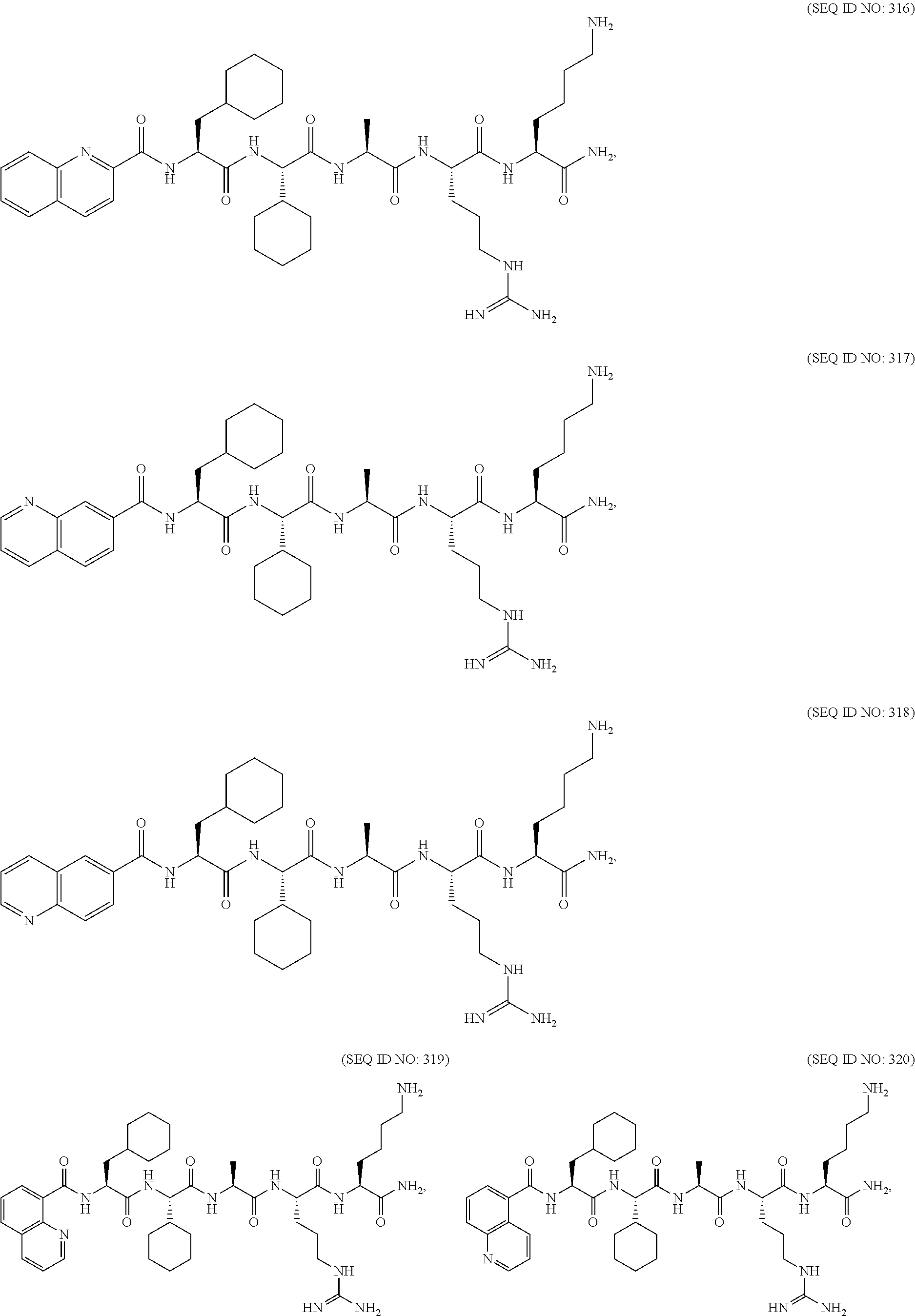

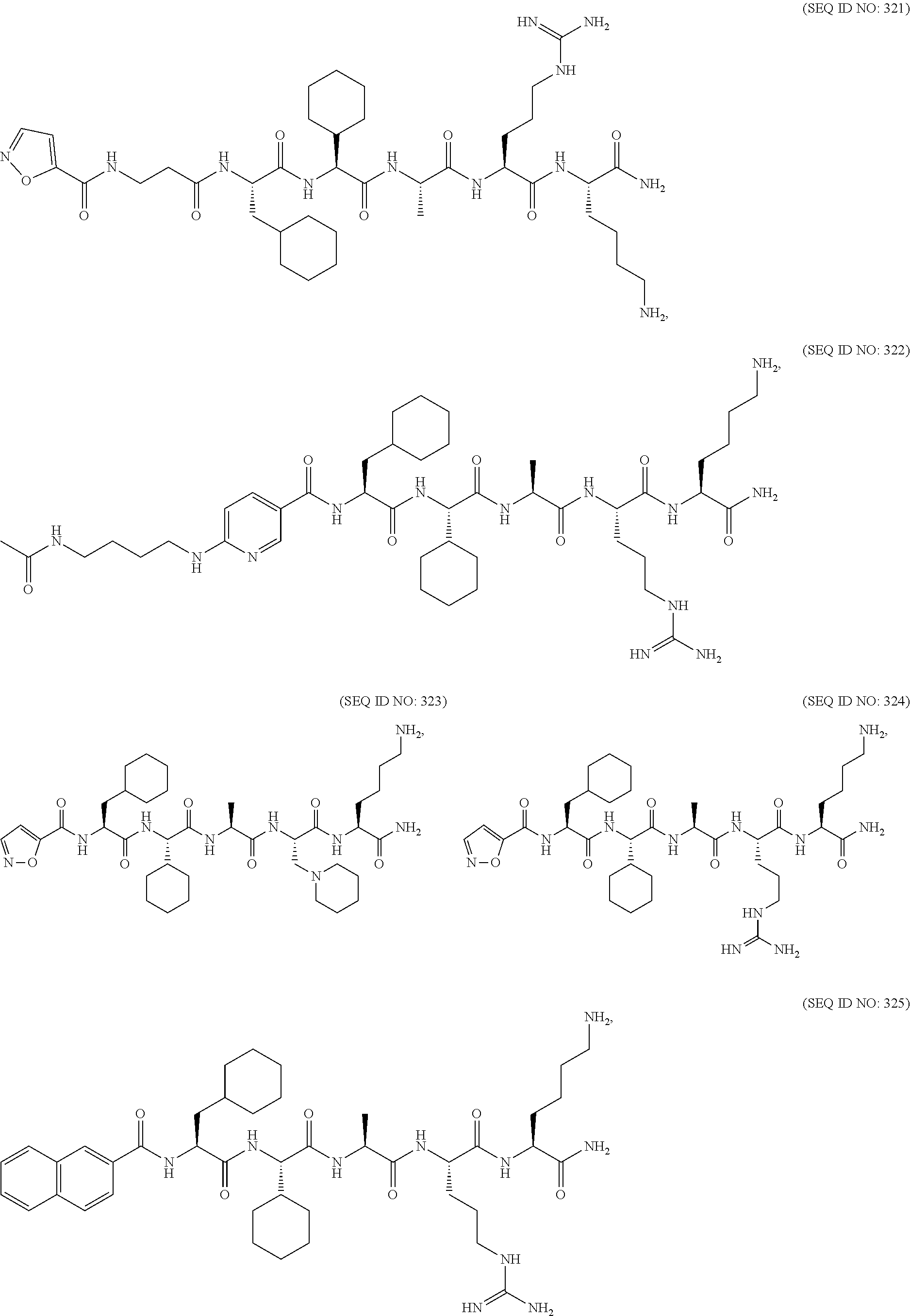

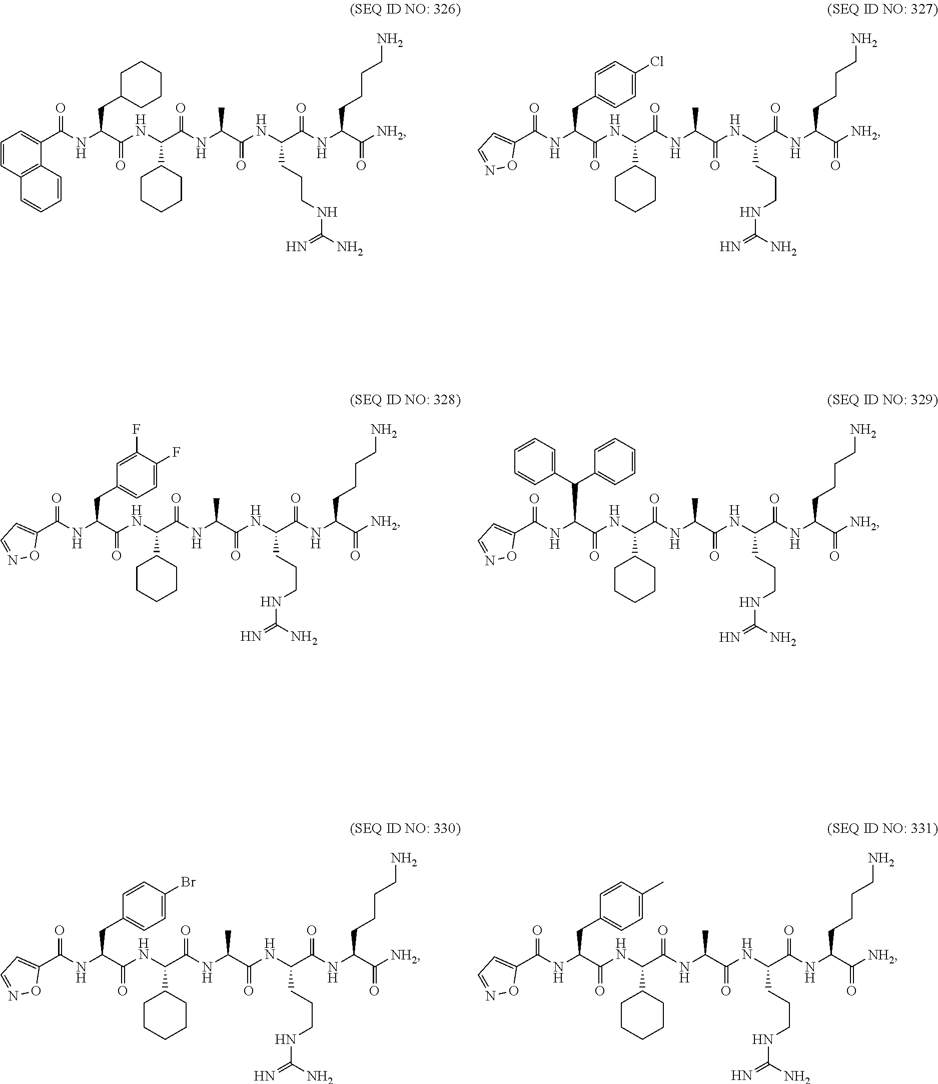

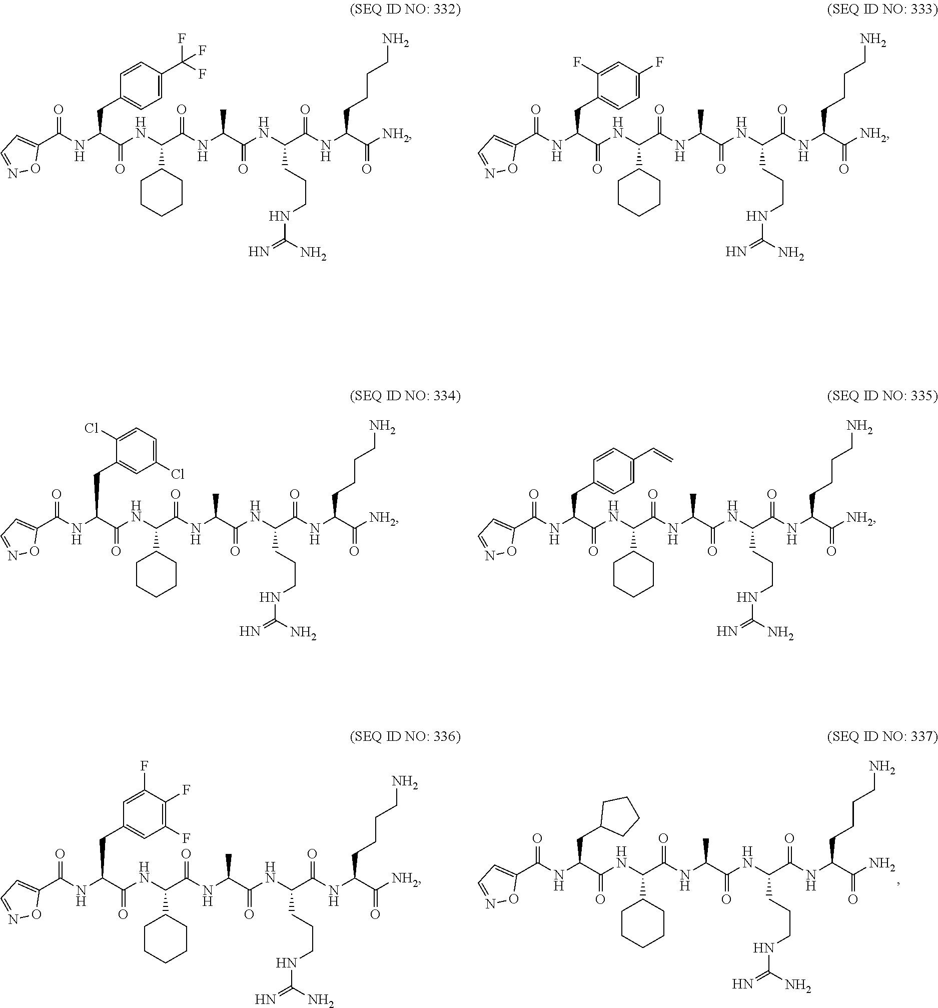

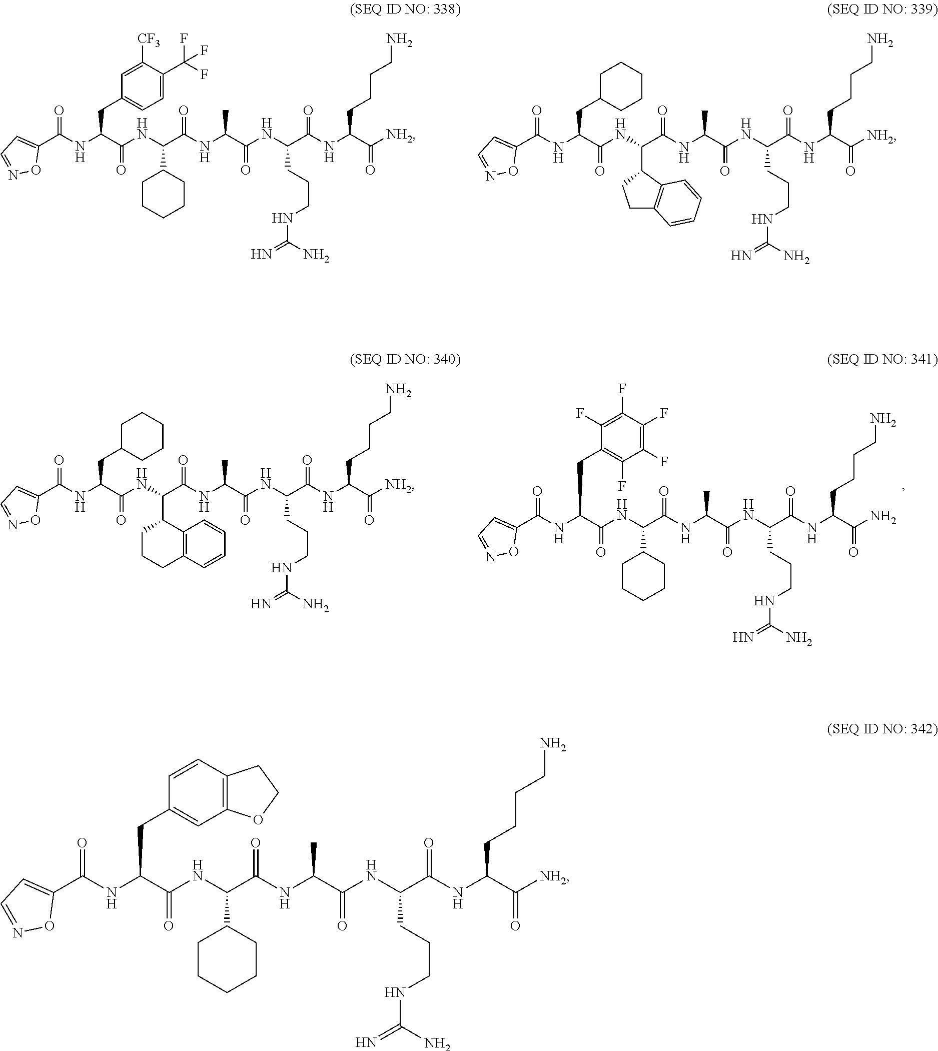

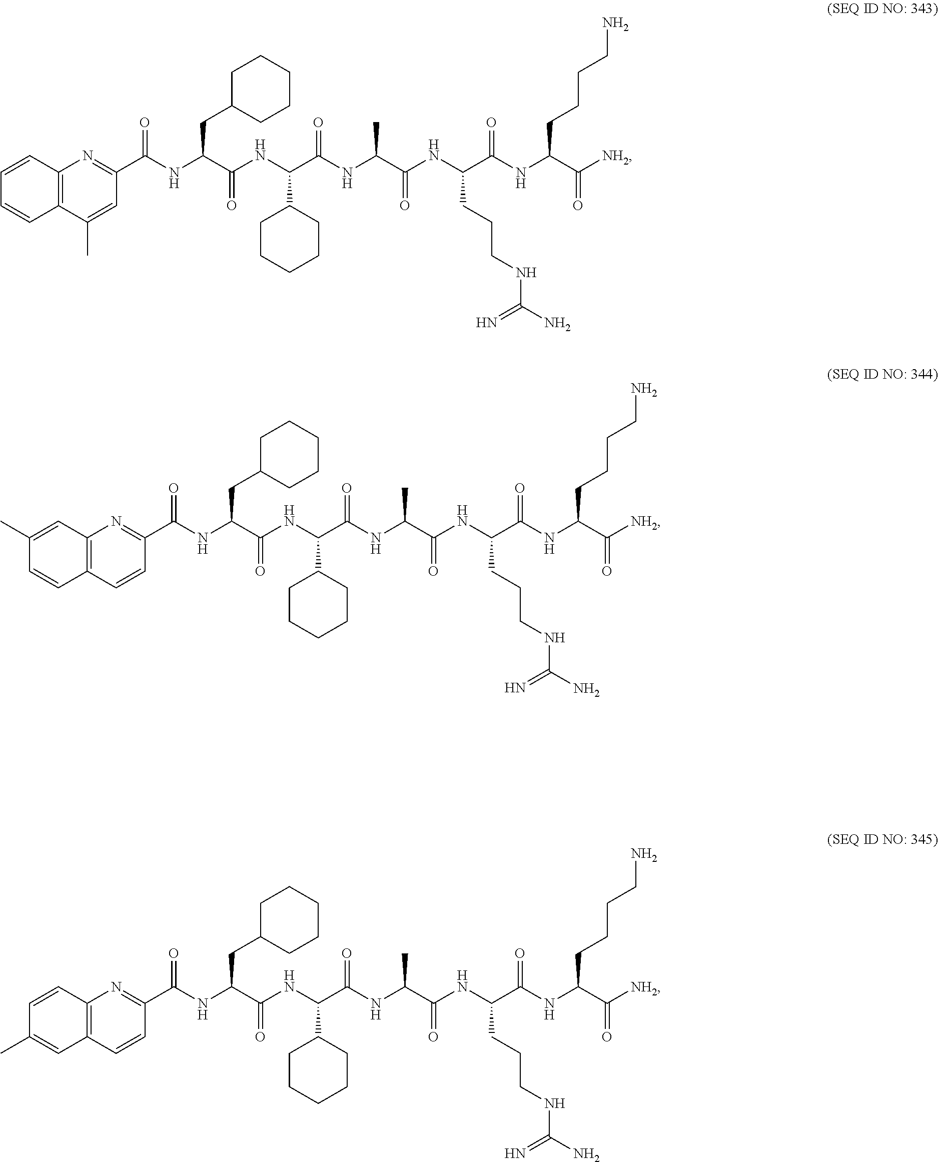 [SMILES: Cc1cc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)nc2ccccc12 ; Cc1ccc2ccc(C(=O)N[C@@H](CC3CCCCC3)C(=O)N[C@H](C(=O)N[C@@H](C)C(=O)N[C@@H](CCCNC(=N)N)C(=O)N[C@@H](CCCCN)C(N)=O)C3CCCCC3)nc2c1 ; Cc1ccc2nc(C(=O)N[C@@H](CC3CCCCC3)C(=O)N[C@H](C(=O)N[C@@H](C)C(=O)N[C@@H](CCCNC(=N)N)C(=O)N[C@@H](CCCCN)C(N)=O)C3CCCCC3)ccc2c1]

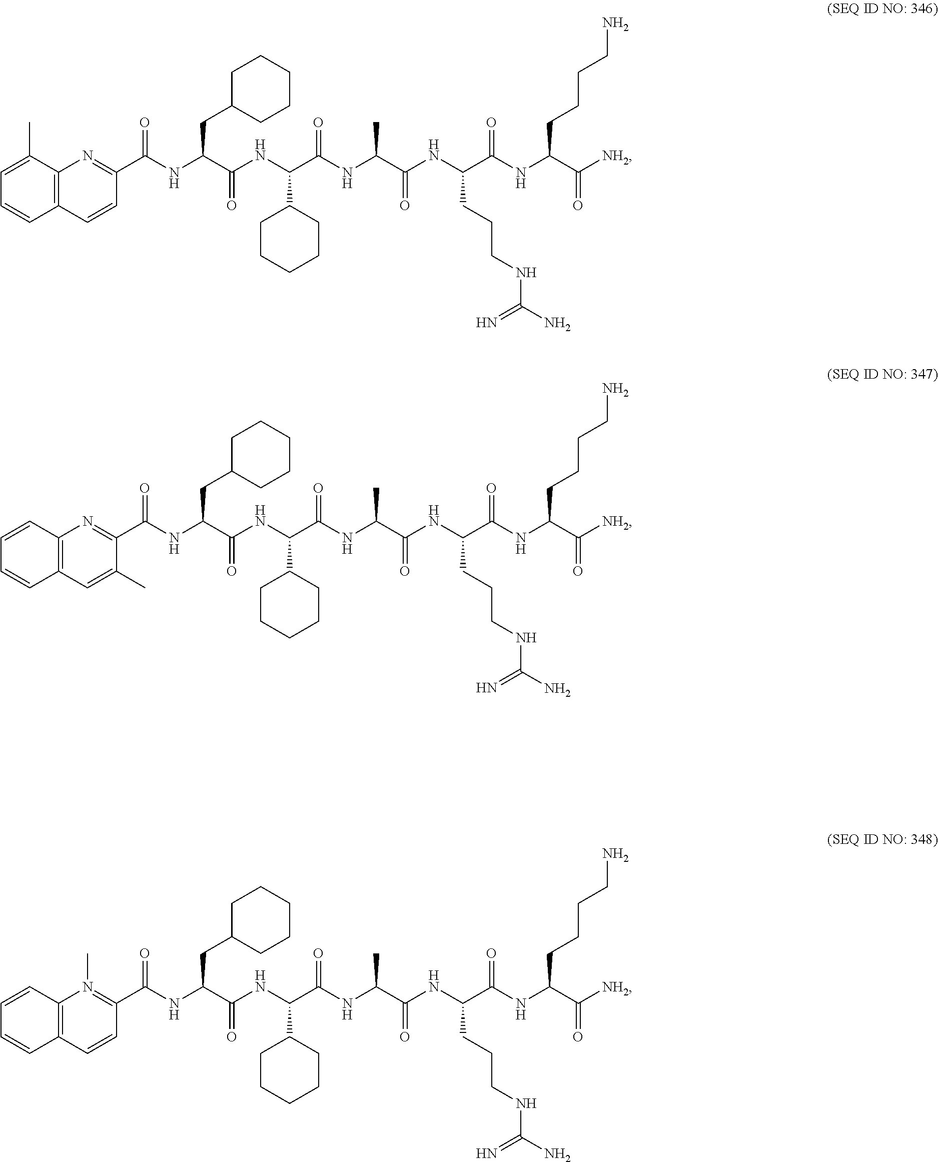

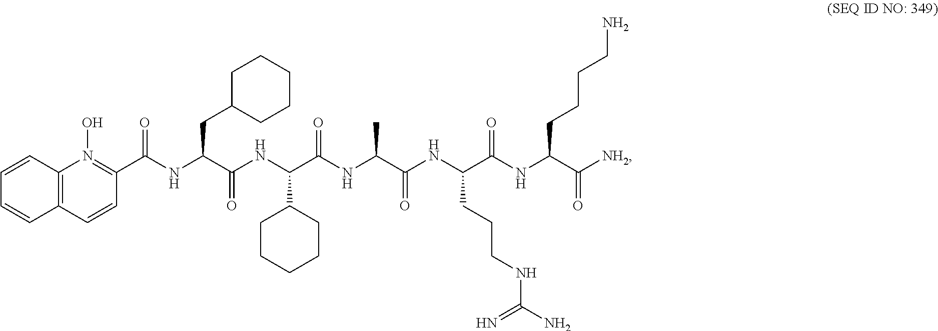

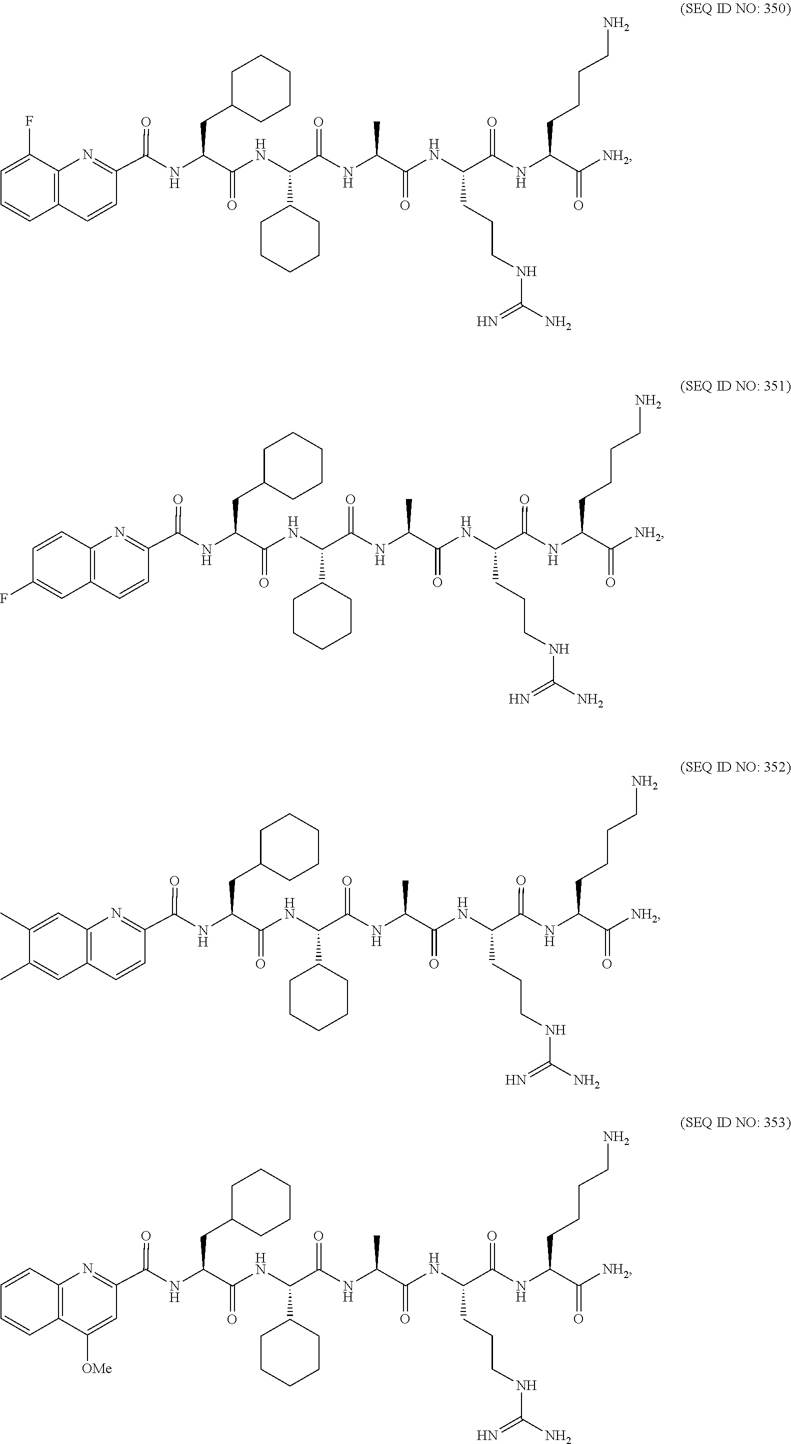

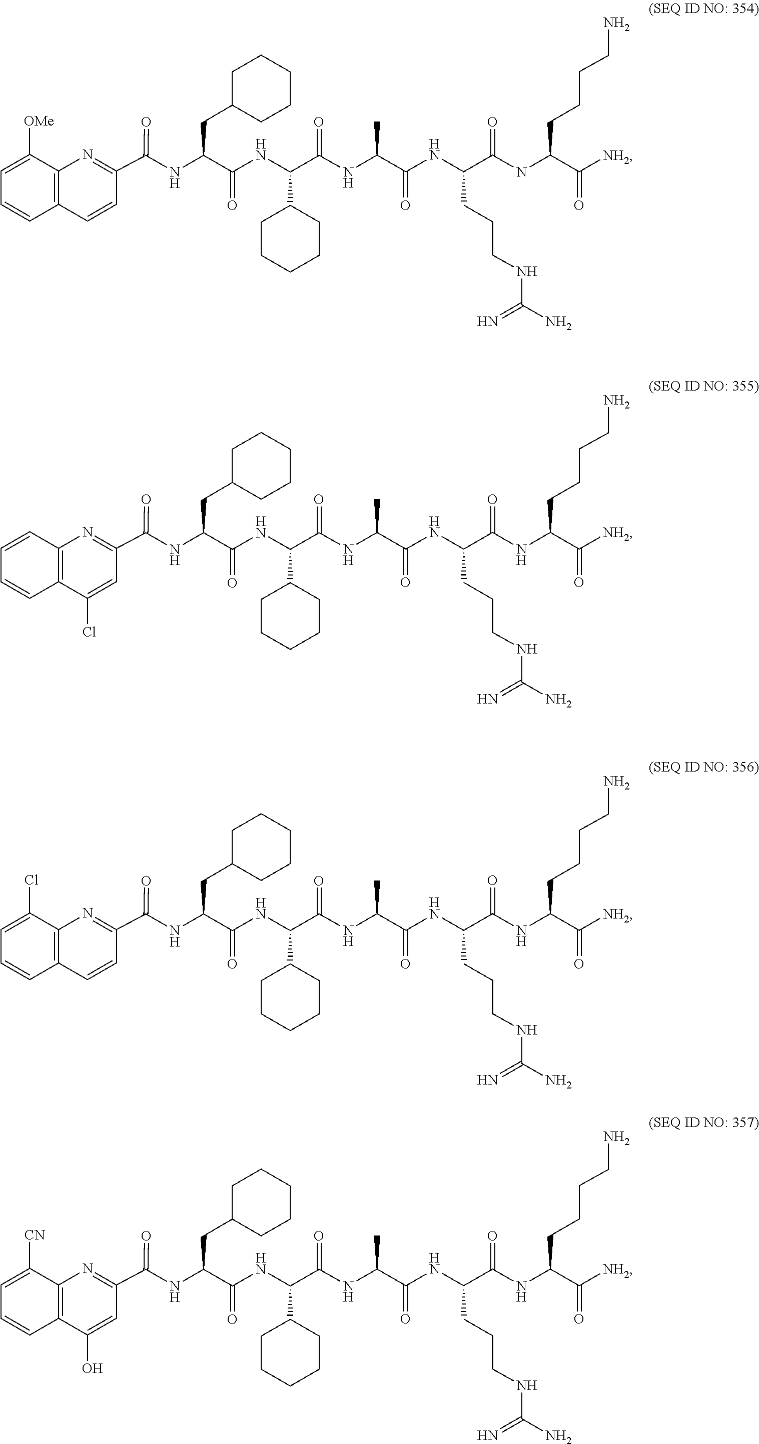

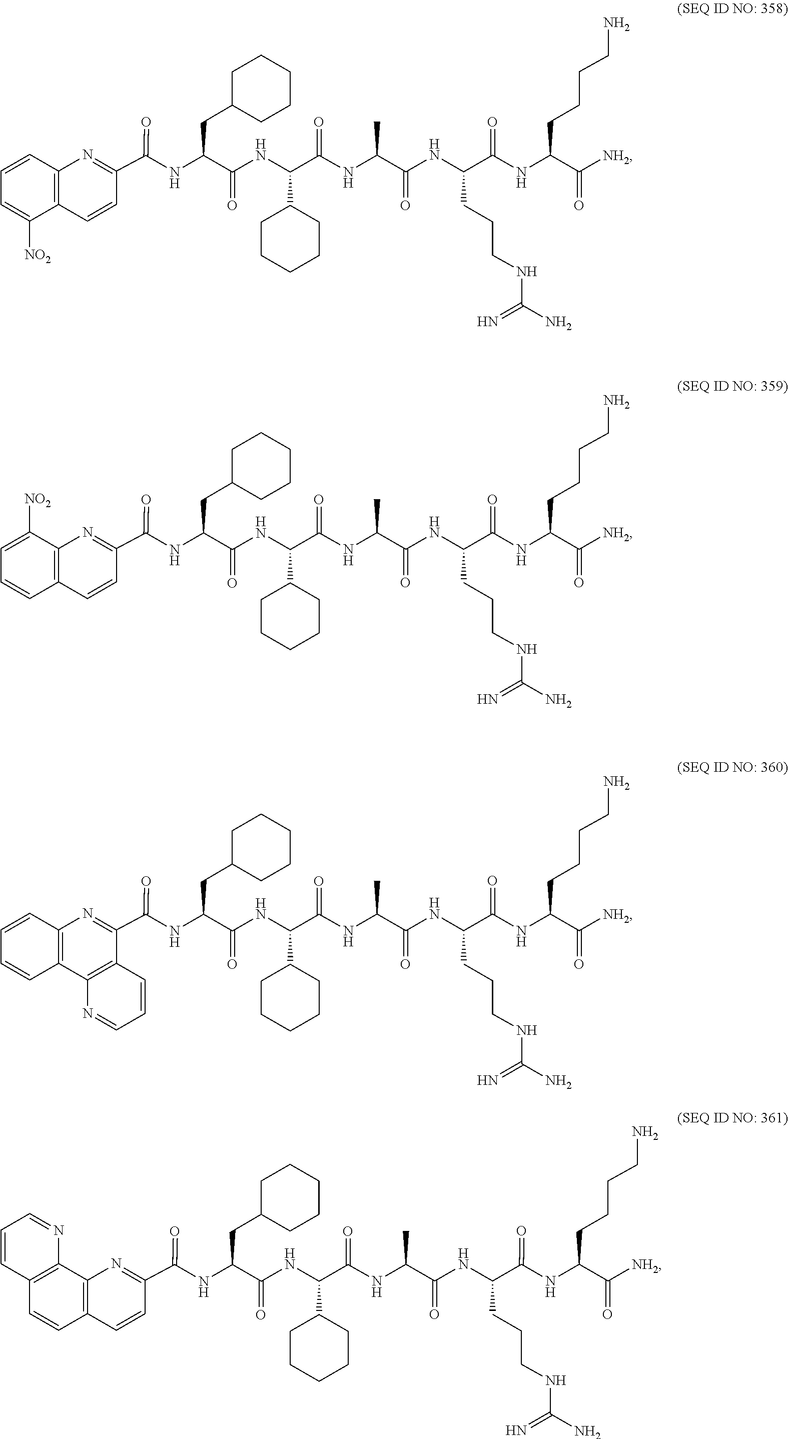

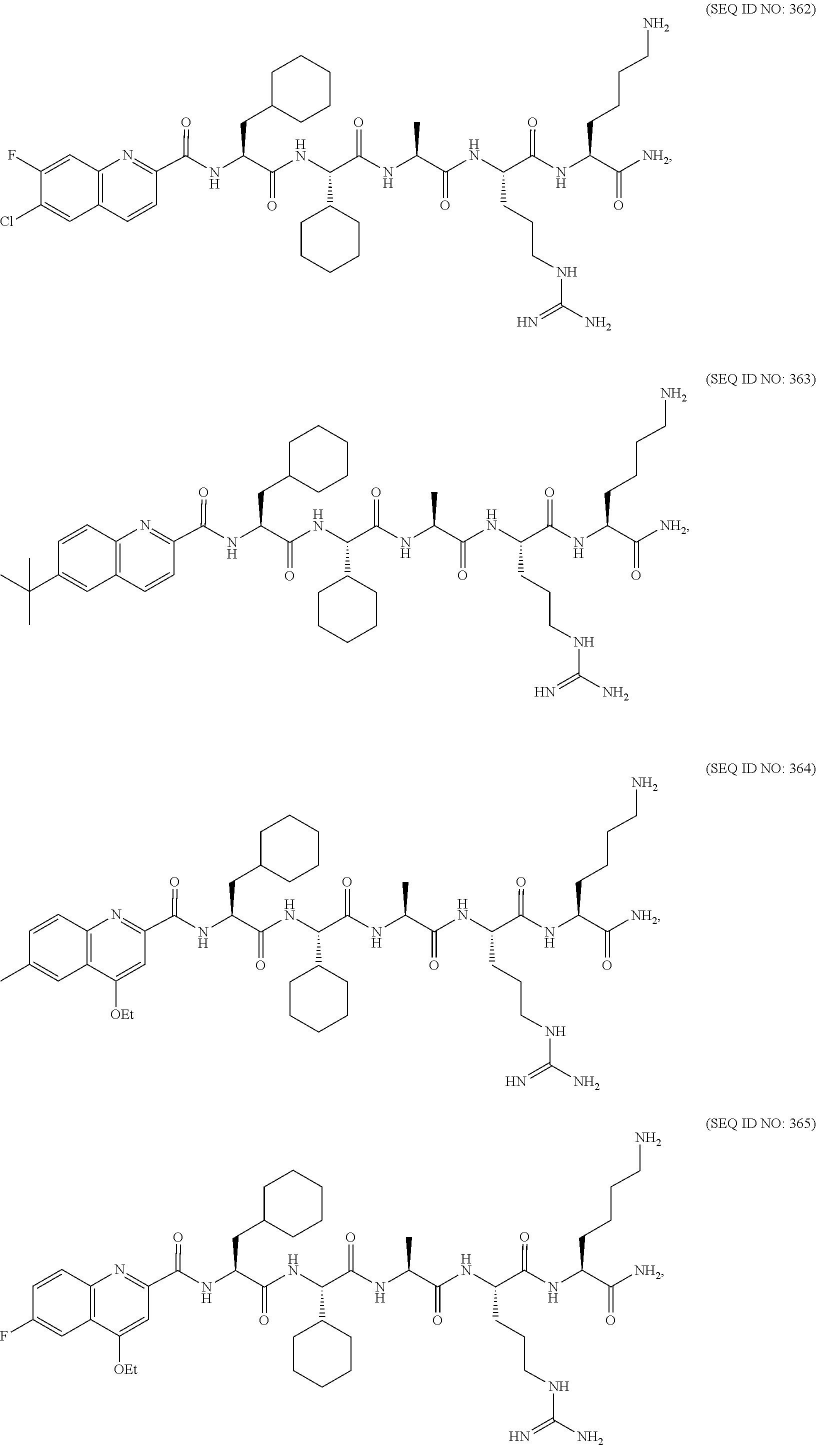

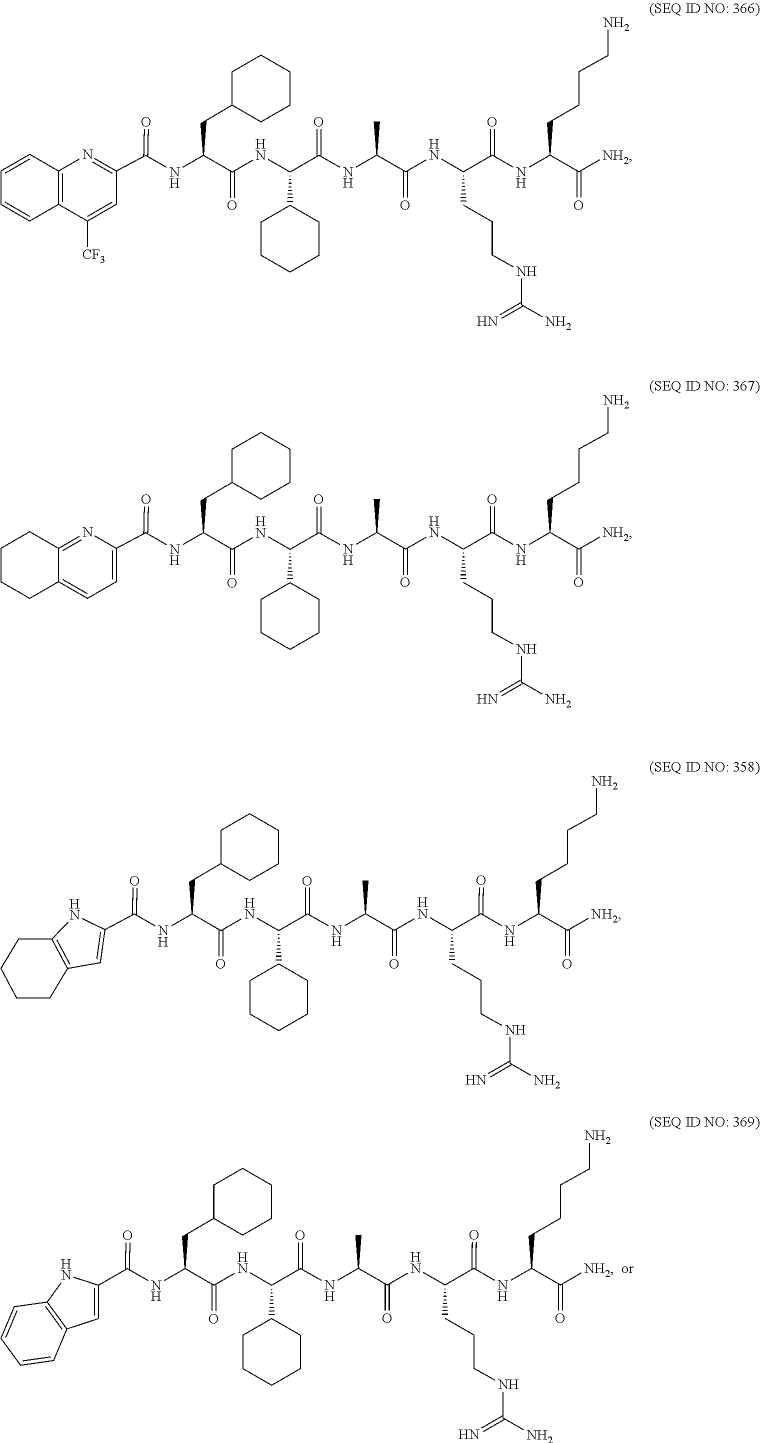

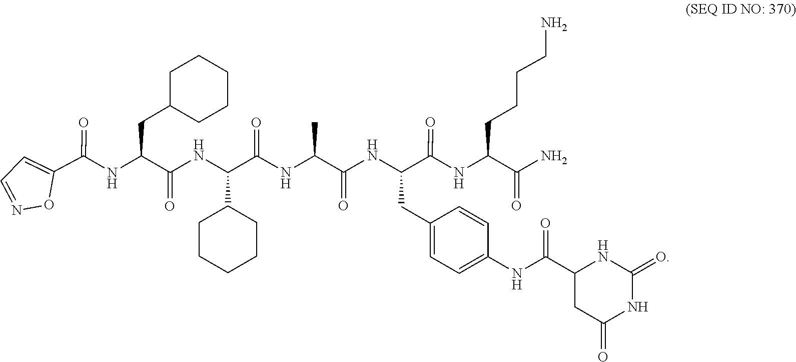 [SMILES: C[C@H](NC(=O)[C@@H](NC(=O)[C@H](CC1CCCCC1)NC(=O)c1ccno1)C1CCCCC1)C(=O)N[C@@H](Cc1ccc(NC(=O)C2CC(=O)NC(=O)N2)cc1)C(=O)N[C@@H](CCCCN)C(N)=O]

Embodiment 139. A pharmaceutical composition comprising a compound of any one of embodiments 89-138, or a pharmaceutically acceptable salt thereof, and at least one pharmaceutically acceptable excipient.

Embodiment 140. The pharmaceutical composition of embodiment 139, wherein the pharmaceutical composition is formulated for administration to a mammal by oral administration.

Embodiment 141. A method for the treatment of a chronic inflammatory condition comprising administering to a mammal with a chronic inflammatory condition an effective amount of a compound of any one of embodiments 89-138, or a pharmaceutically acceptable salt thereof.

Embodiment 142. The method of embodiment 141, wherein the chronic inflammatory condition is atopic dermatitis, asthma, or inflammatory bowel disease.

Embodiment 143. A method for the treatment of direct tissue damage comprising administering to a mammal with tissue damage an effective amount of a compound of any one of embodiments 89-138, or a pharmaceutically acceptable salt thereof.

Embodiment 144. The method of embodiment 143, wherein the direct tissue damage is caused by injury.

Embodiment 145. Use of a compound of Formula (VII) in the preparation of a bifunctional molecule, wherein the bifunctional molecule comprises:
A) a radionuclide conjugate comprising a compound of Formula (VII), an optional linker attached to the Compound of Formula (VII), a chelating moiety attached to the linker, and optionally a radionuclide complexed by the chelating moiety;
B) a peptide-drug conjugate comprising a compound of Formula (VII), an optional linker attached to the Compound of Formula (VII), and a toxin attached to the optional linker; or
C) a peptide-antibody conjugate consisting of a compound of Formula (VII), an optional linker attached to the Compound of Formula (VII), and an antibody attached to the optional linker.

### EXAMPLES

The following examples are provided for illustrative purposes only and not to limit the scope of the claims provided herein.

### Abbreviations

ACN or MeCN or CH₃CN: acetonitrile;
AcOH: acetic acid;
BH₃-THF: borane-tetrahydrofuran;
Boc₂O: di-tert-butyl dicarbonate;
brine: saturated NaCl solution;
BOP: Benzotriazole-1-yl-oxy-tris-(dimethylamino)-phosphonium hexafluorophosphate;
CTC resin: chlorotrityl chloride resin:
CuI: copper (I) iodide;
CuSO₄ 5H₂O: copper (II) sulfate pentahydrate;
DCC: dicyclohexylcarbodiimide;
DCM: dichloromethane;
DHP resin: dihydropyranylmethoxymethyl resin;
DODT: 3,6-dioxa-1,8-octanedithiol;
DIEA or DIPEA: N,N-diisopropylethylamine;
DMEM: Dulbecco's Modified Eagle's Medium;
DMF: dimethylformamide;
DMSO: dimethyl sulfoxide;
DOTA: 2,2′,2″,2″′-(1,4,7,10-tetraazacyclododecane-1,4,7,10-tetrayl)tetraacetic acid or 1,4,7,10-tetraazacyclododecane-1,4,7,10-tetraacetic acid;
DOTA-OSu: 2,2′,2″-(10-(2-((2,5-dioxopyrrolidin-1-yl)oxy)-2-oxoethyl)-1,4,7,10-tetraazacyclododecane-1,4,7-triyl)triacetic acid;
EDC: (1-ethyl-3-(3-dimethylaminopropyl)carbodiimide hydrochloride);
EGTA: Ethyleneglycol-bis(β-aminoethyl)-N N,N′,N′-tetraacetic acid;
EtOAc or EA: ethyl acetate;
EtOH: ethanol;
Et₃N or TEA: triethylamine;
Fmoc: fluorenylmethoxycarbonyl;
Fmoc-OSu: N-(9-Fluorenylmethoxycarbonyloxy)succinimide;
HATU: 1-[Bis(dimethylamino)methylene]-1H-1,2,3-triazolo[4,5-b]pyridinium 3-oxide hexafluorophosphate;
HCl: hydrochloric acid or hydrochloride;
Hex: hexanes;
H₂O: water;
HBTU: N,N,N′,N′-Tetramethyl-O-(1H-benzotriazol-1-yl)uronium hexafluorophosphate;
HOBt: hydroxybenzotriazole;
HMBA: hydroxymethyl benzoic acid resin;
HPLC: high-performance liquid chromatography;
InCl₃: indium trichloride;
LCMS: Liquid chromatography-mass spectrometry;
LuCl₃: lutetium (III) chloride;
MBHA resin: methylbenzhydryl amine resin;
MeOH: methanol;
MPLC: Medium pressure liquid chromatography;
MS: mass spectrometry;
NaBH₄: sodium borohydride;
NaOAc: sodium acetate;
NaOH: sodium hydroxide;
Na₂SO₄: sodium sulfate;
NH₄OAc: ammonium acetate;
NMP: N-Methyl-2-pyrrolidone;
Pd/C: palladium on carbon;
Pd(PPh₃)₄: Tetrakis(triphenylphosphine)palladium(0);
PhSiH₃: phenylsilane;
Prep-HPLC: preparative high-performance liquid chromatography;
RP-HPLC: reversed-phase high-performance liquid chromatography;
SPPS: solid-phase peptide synthesis;
TFA: trifluoroacetic acid;
THF: tetrahydrofuran;
TIS: triisopropylsilane;
rt: room temperature;
hrs: hours; h or hr: hour;
min: minute;
mg: milligrams; kg: kilograms;
mL or ml: milliliter;
Eq: equivalents;
mmol: millimole; mol: moles;
UV: ultraviolet;
v/v: volume/volume.

### Instruments and Materials

Solid phase peptide synthesis was carried out with a Syro II (Biotage) and/or manual shaker using Fmoc chemistry unless otherwise noted.

N^{α}-Fmoc-Rink amide resin and N-Fmoc-Sieber amide resin were purchased from Novabiochem. Amino acid building blocks, solvents and reagents were obtained from commercial suppliers and used without further purification unless otherwise mentioned.

RP-HPLC purifications were performed on a Waters PrepLC system equipped with a Waters 2767 Sample Manager and an XSelect CSH C18 column (5 μm, 19 mm×250 mm) at room temperature. HPLC solvents consisted of H₂O containing 0.1% trifluoroacetic acid or formic acid (mobile phase A) and acetonitrile (mobile phase B) unless otherwise noted.

HPLC analyses were performed on an Agilent 1260 system equipped with a binary pump G7104C, a micro vacuum degasser, a standard auto sampler G7129C, a thermostatted column compartment G7116A, a variable wavelength detector G7117C, and an XBridge Shield RP18 column (3.5 μm, 100 Å, 4.6×150 mm). Data were analyzed by Lab Solutions network workstation software from Shimadzu LCMS-2020. HPLC solvents consisted of H₂O containing 0.05% trifluoroacetic acid (mobile phase A) and acetonitrile (mobile phase B).

LC-MS analyses were carried out on a Shimadzu LCMS-2020 system equipped with a binary pump LC-40D XR, a micro vacuum degasser, a standard auto sampler SIL-40C XR, a thermostatted column compartment CTO-40C, a variable wavelength detector SPD-M20A and a Halo C18 (2.0 μm, 90 Å, 3.0×30 mm) or a CORTECS T3 (2.7 μm, 120 Å, 2.1×30 mm) column. Data were analyzed by Lab Solutions standalone workstation software from Agilent Technologies. HPLC solvents consisted of H₂O containing 0.1% formic acid (mobile phase A) and acetonitrile (mobile phase B).

MPLC purifications were performed with an Orinedia preparative HPLC (BRIX 2802) on silica gel columns.

¹H NMR spectra were recorded using an AVANCE III HD 300 MHz, AVANCE NEO 400 MHz, or Bruker 300 MHz or 400 MHz. Chemical shifts are reported in δ (ppm) relative to TMS₄Si (in CDCl₃) as internal standard using Bruker TopSpin software unless otherwise noted.

A peptide of the present disclosure may be prepared through known methods, including procedures referenced in “Binding of a highly potent protease-activated receptor-2 (PAR2) activating peptide, [3H]2-furoyl-LIGRL-NH₂, to human PAR2” Kanke et al., British Journal of Pharmacology 145: 255-263 (2006); “A refined agonist pharmacophore for protease activated receptor 2” Barry et al., Bioorganic & Medicinal Chemistry, Letters 17: 5552-5557 (2007); “Development of highly potent protease-activated receptor 2 agonists via synthetic lipid tethering” Flynn et al., The FASEB Journal 27: 1498-1510 (2013); “Potent Small Agonists of Protease Activated Receptor 2” Yau et al., ACS Medicinal Chemistry Letters 7: 105-110 (2015); “Biased signaling by peptide agonists of protease activated receptor 2” Jiang et al., ACS Chemical Biology, 12: 1217-1226 (2017); “High Affinity Fluorescent Probe for Proteinase-Activated Receptor 2 (PAR2)” LeSarge et al., ACS Medicinal Chemistry Letters 10: 1045-1050 (2019); or “Protease-activated receptor 2 (PAR2)-targeting peptide derivatives for positron emission tomography (PET) imaging” LeSarge et al., European Journal of Medicinal Chemistry 246: 114989 (2023).

### Procedure A: General Procedure for Solid-Phase Peptide Synthesis SPPS

Resin swelling: the resin (100 mg) was swelled in NMP (1 mL) for 15 min at room temperature under a nitrogen atmosphere. The resin was then washed 4 times with NMP (1 mL).

Fmoc de-protection: the resin (100 mg) was treated with 20% piperidine in NMP (1 mL) for 20 min at room temperature under a nitrogen atmosphere. Then, the resin was washed 4 times with NMP (1 mL).

HATU coupling: the resin (100 mg) was treated with the mixture of amino acid (4.0 Eq), HATU (4.0 Eq) and DIPEA (8.0 Eq) in NMP (1 mL) for 45 min at 30° C. under a nitrogen atmosphere unless otherwise mentioned. Then, the resin was washed 4 times with NMP (1 mL).

Cleavage & purification: a suspension of the resin in the cleavage solution TFA/H₂O/TIS (37:1:1, v/v/v) or TFA/TIS/DODT/DCM (10:1:1:28, v/v/v/v) was stirred for 1-2 h at room temperature. After the resin was filtered off, the filtrate was concentrated under reduced pressure. To the residue was added cold ether, the precipitate was collected by centrifugation and purified by RP-HPLC. The desired fractions were combined and dried by lyophilization.

### Procedure B: General Synthesis Procedure for Metal Complexation

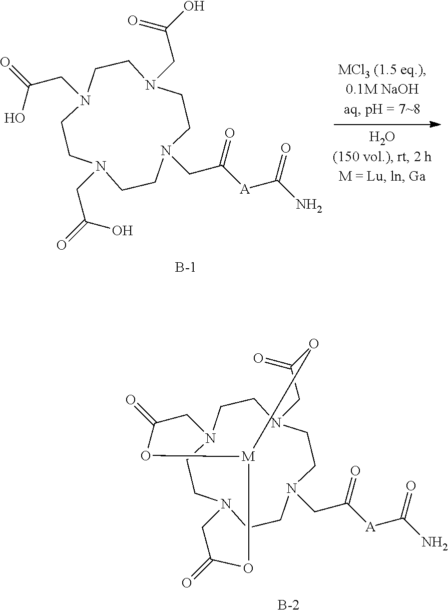

Compound B-i (1.0 Eq) and MCl₃ (1.5 Eq) were dissolved in H₂O (150.0 vol). The mixture was neutralized with 0.1 M aq. NaOH to pH of ~7-8. The mixture was stirred for 2 hours at room temperature and monitored by LCMS. Upon completion, the mixture was centrifuged, and the precipitate was purified by prep-HPLC and dried by lyophilization to afford B-2.

### Procedure C: General Synthesis Procedure for ¹¹¹In-Labeling

[¹¹¹In]InCl₃ in HCl was added to a solution of a ligand in NH₄OAc or NaOAc buffer (0.1 M, pH 5.0-5.5). The resulting mixture was heated at 60-95° C. in a thermal mixer for 15-30 minutes. Radiochemical purity was determined using iTLC or radioHPLC analytical methods. The typical molar activities used in the studies ranged from 5-7 MBq/nmol to 10-15 MBq/nmol. The radiotracer solution for in vivo studies was formulated by dilution of the reaction mixture with 0.9% saline containing proper excipients based on stability studies.

Examples disclosed herein were prepared according to the general procedures outlined above, and the specific procedures that follow.

### EXAMPLES

Compounds 1-210 were synthesized according to Procedure A: General Procedure for SPPS using the appropriate amino acid building blocks.

### Example 1 (Compound 1) (SEQ ID NO: 161)

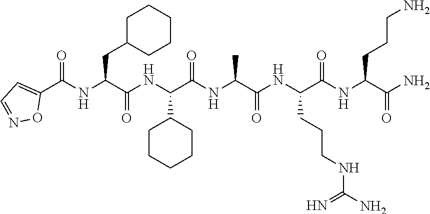 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCN)C(N)=O]

MS: Calc'd for C₃₅H₅₉N₁₁O₇: 745.46, found [M+2H]²⁺: 746.4.

### Example 2 (Compound 2) (SEQ ID NO: 162)

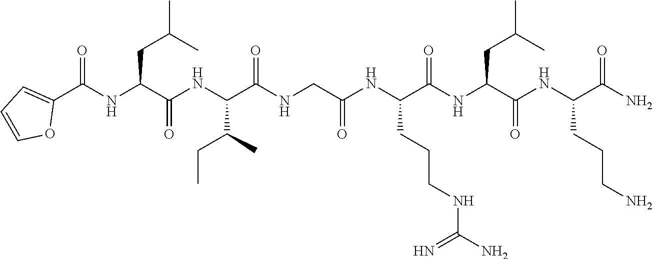 [SMILES: CC[C@H](C)[C@H](NC(=O)[C@H](CC(C)C)NC(=O)c1ccco1)C(=O)NCC(=O)N[C@@H](CCCNC(=N)N)C(=O)N[C@@H](CC(C)C)C(=O)N[C@@H](CCCN)C(N)=O]

### Example 3 (Compound 3) (SEQ ID NO: 163)

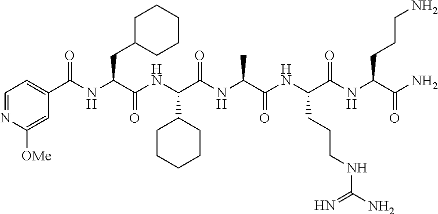 [SMILES: COc1cc(C(=O)N[C@@H](CC2CCCCC2)C(=O)N[C@H](C(=O)N[C@@H](C)C(=O)N[C@@H](CCCNC(=N)N)C(=O)N[C@@H](CCCN)C(N)=O)C2CCCCC2)ccn1]

MS: Calc'd for C₃₈H₆₃N₁₁O₇: 785.49, found [M+H]⁺: 786.4.

### Example 4 (Compound 4) (SEQ ID NO: 164)

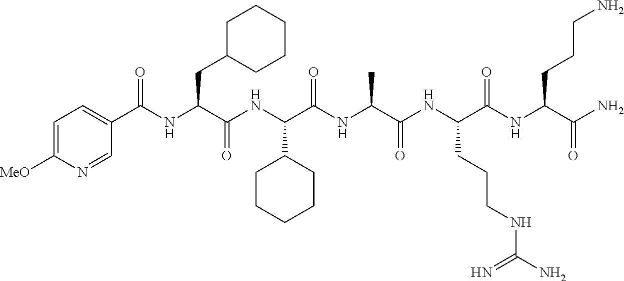 [SMILES: COc1ccc(C(=O)N[C@@H](CC2CCCCC2)C(=O)N[C@H](C(=O)N[C@@H](C)C(=O)N[C@@H](CCCNC(=N)N)C(=O)N[C@@H](CCCN)C(N)=O)C2CCCCC2)cn1]

MS: Calc'd for C₃₈H₆₃N₁₁O₇: 785.49, found [M+H]⁺: 786.4.

### Example 5 (Compound 5) (SEQ ID NO: 165)

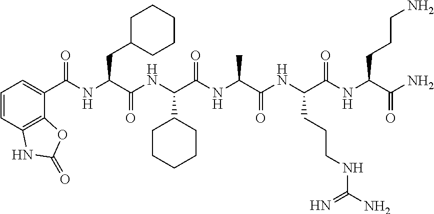 [SMILES: C[C@H](NC(=O)[C@@H](NC(=O)[C@H](CC1CCCCC1)NC(=O)c1cccc2[nH]c(=O)oc12)C1CCCCC1)C(=O)N[C@@H](CCCNC(=N)N)C(=O)N[C@@H](CCCN)C(N)=O]

MS: Calc'd for C₃₉H₆₁N₁₁O₈: 811.47, found [M+H]⁺: 812.4.

### Example 6 (Compound 6) (SEQ ID NO: 166)

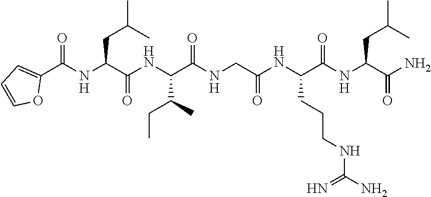 [SMILES: CC[C@H](C)[C@H](NC(=O)[C@H](CC(C)C)NC(=O)c1ccco1)C(=O)NCC(=O)N[C@@H](CCCNC(=N)N)C(=O)N[C@@H](CC(C)C)C(N)=O]

MS: Calc'd for C₃₁H₅₃N₉O₇: 663.41, found [M+H]⁺: 664.3.

### Example 7 (Compound 7) (SEQ ID NO: 167)

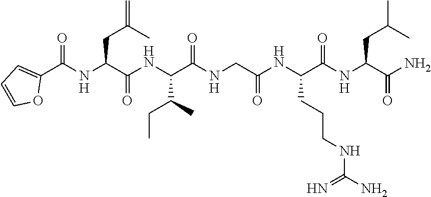 [SMILES: C=C(C)C[C@H](NC(=O)c1ccco1)C(=O)N[C@H](C(=O)NCC(=O)N[C@@H](CCCNC(=N)N)C(=O)N[C@@H](CC(C)C)C(N)=O)[C@@H](C)CC]

MS: Calc'd for C₃₁H₅₁N₉O₇: 661.39, found [M+H]⁺: 662.2.

### Example 8 (Compound 8) (SEQ ID NO: 168)

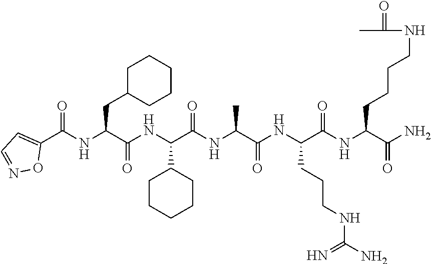 [SMILES: CC(=O)NCCCC[C@H](NC(=O)[C@H](CCCNC(=N)N)NC(=O)[C@H](C)NC(=O)[C@@H](NC(=O)[C@H](CC1CCCCC1)NC(=O)c1ccno1)C1CCCCC1)C(N)=O]

MS: Calc'd for C₃₈H₆₃N₁₁O₈: 801.49, found [M+H]⁺: 802.5.

### Example 9 (Compound 9) (SEQ ID NO: 169)

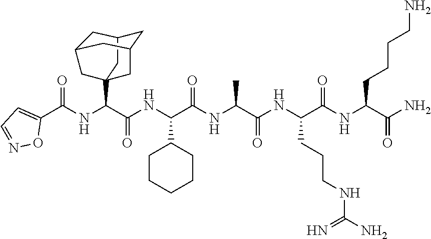 [SMILES: C[C@H](NC(=O)[C@@H](NC(=O)[C@@H](NC(=O)c1ccno1)C12CC3CC(CC(C3)C1)C2)C1CCCCC1)C(=O)N[C@@H](CCCNC(=N)N)C(=O)N[C@@H](CCCCN)C(N)=O]

MS: Calc'd for C₃₉H₆₃N₁₁O₇: 797.49, found [M+2H]²⁺: 400.0.

### Example 10 (Compound 10) (SEQ ID NO: 170)

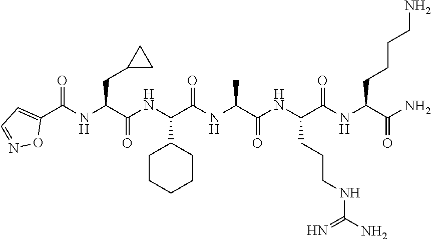 [SMILES: C[C@H](NC(=O)[C@@H](NC(=O)[C@H](CC1CC1)NC(=O)c1ccno1)C1CCCCC1)C(=O)N[C@@H](CCCNC(=N)N)C(=O)N[C@@H](CCCCN)C(N)=O]

MS: Calc'd for C₃₃H₅₅N₁₁O₇: 717.43, found [M+2H]²⁺: 359.9.

### Example 11 (Compound 11) (SEQ ID NO: 171)

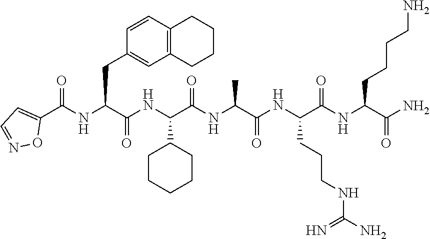 [SMILES: C[C@H](NC(=O)[C@@H](NC(=O)[C@H](Cc1ccc2c(c1)CCCC2)NC(=O)c1ccno1)C1CCCCC1)C(=O)N[C@@H](CCCNC(=N)N)C(=O)N[C@@H](CCCCN)C(N)=O]

### Example 12 (Compound 12) (SEQ ID NO: 172)

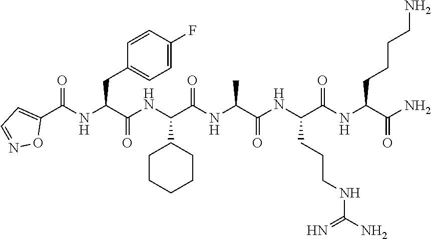 [SMILES: C[C@H](NC(=O)[C@@H](NC(=O)[C@H](Cc1ccc(F)cc1)NC(=O)c1ccno1)C1CCCCC1)C(=O)N[C@@H](CCCNC(=N)N)C(=O)N[C@@H](CCCCN)C(N)=O]

MS: Calc'd for C₃₆H₅₄FN₁₁O₇: 771.42, found [M+2H]²⁺: 387.0.

### Example 13 (Compound 13) (SEQ ID NO: 173)

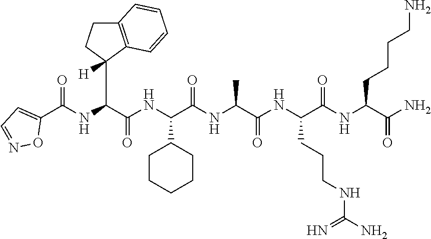 [SMILES: C[C@H](NC(=O)[C@@H](NC(=O)[C@@H](NC(=O)c1ccno1)[C@@H]1CCc2ccccc21)C1CCCCC1)C(=O)N[C@@H](CCCNC(=N)N)C(=O)N[C@@H](CCCCN)C(N)=O]

### Example 14 (Compound 14) (SEQ ID NO: 174)

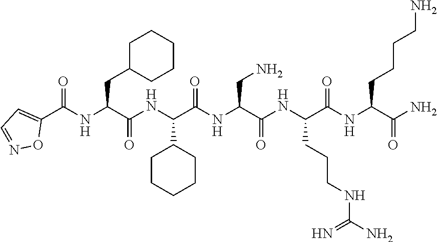 [SMILES: N=C(N)NCCC[C@H](NC(=O)[C@H](CN)NC(=O)[C@@H](NC(=O)[C@H](CC1CCCCC1)NC(=O)c1ccno1)C1CCCCC1)C(=O)N[C@@H](CCCCN)C(N)=O]

MS: Calc'd for C₃₆H₆₂N₁₂O₇: 774.49, found [M+2H]²⁺: 388.5.

### Example 15 (Compound 15) (SEQ ID NO: 175)

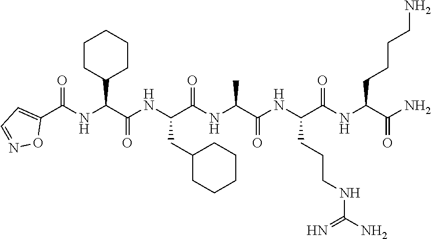 [SMILES: C[C@H](NC(=O)[C@H](CC1CCCCC1)NC(=O)[C@@H](NC(=O)c1ccno1)C1CCCCC1)C(=O)N[C@@H](CCCNC(=N)N)C(=O)N[C@@H](CCCCN)C(N)=O]

MS: Calc'd for C₃₆H₆₁N₁₁O₇: 759.48, found [M+2H]²⁺: 381.0.

### Example 16 (Compound 16) (SEQ ID NO: 176)

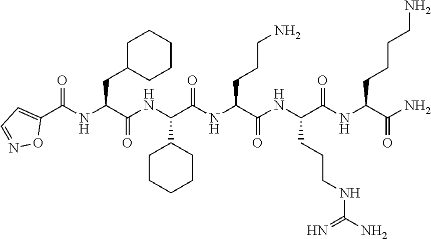 [SMILES: N=C(N)NCCC[C@H](NC(=O)[C@H](CCCN)NC(=O)[C@@H](NC(=O)[C@H](CC1CCCCC1)NC(=O)c1ccno1)C1CCCCC1)C(=O)N[C@@H](CCCCN)C(N)=O]

MS: Calc'd for C₃₈H₆₆N₁₂O₇: 802.52, found [M+2H]²⁺: 402.5.

### Example 17 (Compound 17) (SEQ ID NO: 177)

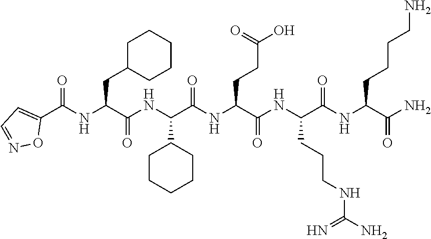 [SMILES: N=C(N)NCCC[C@H](NC(=O)[C@H](CCC(=O)O)NC(=O)[C@@H](NC(=O)[C@H](CC1CCCCC1)NC(=O)c1ccno1)C1CCCCC1)C(=O)N[C@@H](CCCCN)C(N)=O]

MS: Calc'd for C₃₈H₆₃N₁₁O₉: 817.48, found [M+2H]²⁺: 410.1.

### Example 18 (Compound 18) (SEQ ID NO: 178)

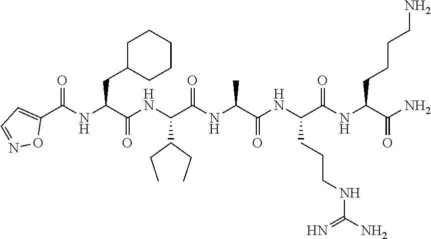 [SMILES: CCC(CC)[C@H](NC(=O)[C@H](CC1CCCCC1)NC(=O)c1ccno1)C(=O)N[C@@H](C)C(=O)N[C@@H](CCCNC(=N)N)C(=O)N[C@@H](CCCCN)C(N)=O]

MS: Calc'd for C₃₅H₆₁N₁O₇: 747.48, found [M+2H]²⁺: 375.0.

### Example 19 (Compound 19) (SEQ ID NO: 179)

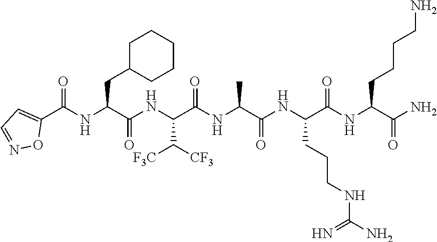 [SMILES: C[C@H](NC(=O)[C@@H](NC(=O)[C@H](CC1CCCCC1)NC(=O)c1ccno1)C(C(F)(F)F)C(F)(F)F)C(=O)N[C@@H](CCCNC(=N)N)C(=O)N[C@@H](CCCCN)C(N)=O]

### Example 20 (Compound 20) (SEQ ID NO: 180)

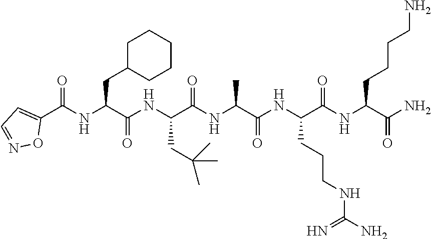 [SMILES: C[C@H](NC(=O)[C@H](CC(C)(C)C)NC(=O)[C@H](CC1CCCCC1)NC(=O)c1ccno1)C(=O)N[C@@H](CCCNC(=N)N)C(=O)N[C@@H](CCCCN)C(N)=O]

MS: Calc'd for C₃₅H₆₁N₁₁O₇: 747.48, found [M+2H]²⁺: 374.9.

### Example 21 (Compound 21) (SEQ ID NO: 181)

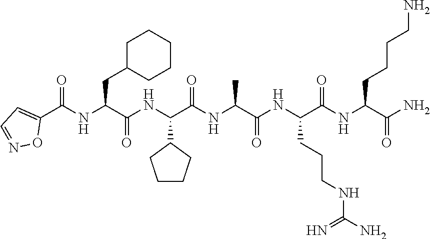 [SMILES: C[C@H](NC(=O)[C@@H](NC(=O)[C@H](CC1CCCCC1)NC(=O)c1ccno1)C1CCCC1)C(=O)N[C@@H](CCCNC(=N)N)C(=O)N[C@@H](CCCCN)C(N)=O]

MS: Calc'd for C₃₅H₅N₁₁O₇: 745.46, found [M+2H]²⁺: 373.9.

### Example 22 (Compound 22) (SEQ ID NO: 182)

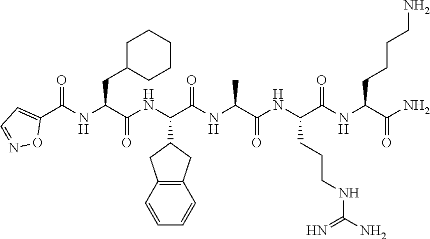 [SMILES: C[C@H](NC(=O)[C@@H](NC(=O)[C@H](CC1CCCCC1)NC(=O)c1ccno1)C1Cc2ccccc2C1)C(=O)N[C@@H](CCCNC(=N)N)C(=O)N[C@@H](CCCCN)C(N)=O]

MS: Calc'd for C₃₉H₁₅₉N₁₁O₇: 793.46, found [M+2H]²⁺: 398.0.

### Example 23 (Compound 23) (SEQ ID NO: 183)

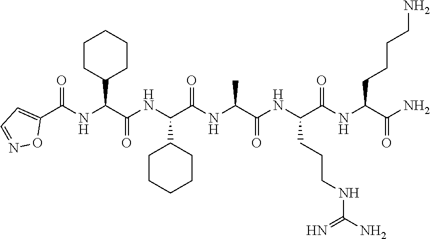 [SMILES: C[C@H](NC(=O)[C@@H](NC(=O)[C@@H](NC(=O)c1ccno1)C1CCCCC1)C1CCCCC1)C(=O)N[C@@H](CCCNC(=N)N)C(=O)N[C@@H](CCCCN)C(N)=O]

MS: Calc'd for C₃₅H₅N₁₁O₇: 745.46, found [M+2H]²⁺: 373.9.

### Example 24 (Compound 24) (SEQ ID NO: 184)

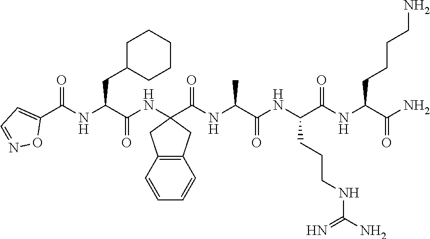 [SMILES: C[C@H](NC(=O)C1(NC(=O)[C@H](CC2CCCCC2)NC(=O)c2ccno2)Cc2ccccc2C1)C(=O)N[C@@H](CCCNC(=N)N)C(=O)N[C@@H](CCCCN)C(N)=O]

MS: Calc'd for C₃₈H₅N₁₁O₇: 779.44, found [M+2H]²⁺: 391.0.

### Example 25 (Compound 25) (SEQ ID NO: 185)

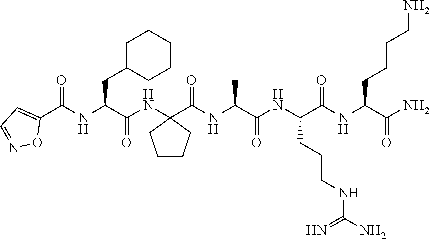 [SMILES: C[C@H](NC(=O)C1(NC(=O)[C@H](CC2CCCCC2)NC(=O)c2ccno2)CCCC1)C(=O)N[C@@H](CCCNC(=N)N)C(=O)N[C@@H](CCCCN)C(N)=O]

MS: Calc'd for C₃₄H₅₇N₁₁O₇: 731.44, found [M+2H]²⁺: 367.0.

### Example 26 (Compound 26) (SEQ ID NO: 186)

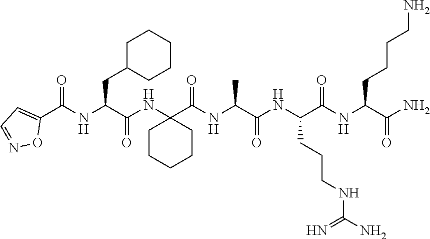 [SMILES: C[C@H](NC(=O)C1(NC(=O)[C@H](CC2CCCCC2)NC(=O)c2ccno2)CCCCC1)C(=O)N[C@@H](CCCNC(=N)N)C(=O)N[C@@H](CCCCN)C(N)=O]

MS: Calc'd for C₃₅H₅N₁₁O₇: 745.46, found [M+2H]²⁺: 374.0.

### Example 27 (Compound 27) (SEQ ID NO: 187)

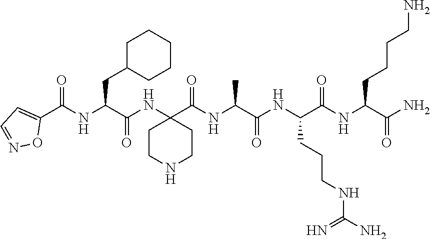 [SMILES: C[C@H](NC(=O)C1(NC(=O)[C@H](CC2CCCCC2)NC(=O)c2ccno2)CCNCC1)C(=O)N[C@@H](CCCNC(=N)N)C(=O)N[C@@H](CCCCN)C(N)=O]

MS: Calc'd for C₃₄H₅₈N₁₂O₇: 746.45, found [M+3H]³⁺: 250.1.

### Example 28 (Compound 28) (SEQ ID NO: 188)

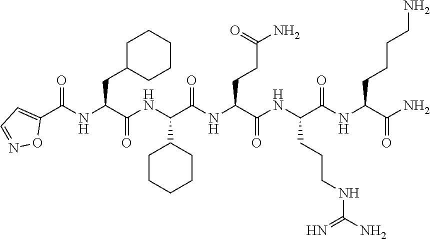 [SMILES: N=C(N)NCCC[C@H](NC(=O)[C@H](CCC(N)=O)NC(=O)[C@@H](NC(=O)[C@H](CC1CCCCC1)NC(=O)c1ccno1)C1CCCCC1)C(=O)N[C@@H](CCCCN)C(N)=O]

MS: Calc'd for C₃₈H₆₄N₁₂O₈: 816.50, found [M+2H]²⁺: 409.5.

### Example 29 (Compound 29) (SEQ ID NO: 189)

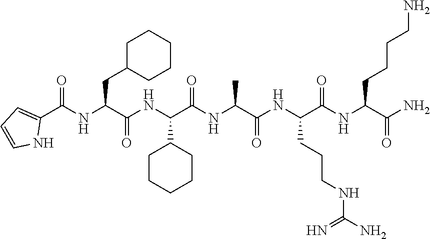 [SMILES: C[C@H](NC(=O)[C@@H](NC(=O)[C@H](CC1CCCCC1)NC(=O)c1ccc[nH]1)C1CCCCC1)C(=O)N[C@@H](CCCNC(=N)N)C(=O)N[C@@H](CCCCN)C(N)=O]

MS: Calc'd for C₃₇H₆₃N₁O₆: 757.50, found [M+2H]²⁺: 380.0.

### Example 30 (Compound 30) (SEQ ID NO: 190)

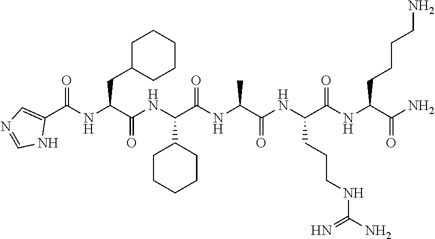 [SMILES: C[C@H](NC(=O)[C@@H](NC(=O)[C@H](CC1CCCCC1)NC(=O)c1cnc[nH]1)C1CCCCC1)C(=O)N[C@@H](CCCNC(=N)N)C(=O)N[C@@H](CCCCN)C(N)=O]

MS: Calc'd for C₃₆H₆₂N₁₂O₆: 758.49, found [M+2H]²⁺: 380.6.

### Example 31 (Compound 31) (SEQ ID NO: 191)

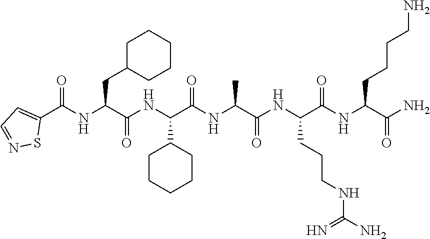 [SMILES: C[C@H](NC(=O)[C@@H](NC(=O)[C@H](CC1CCCCC1)NC(=O)c1ccns1)C1CCCCC1)C(=O)N[C@@H](CCCNC(=N)N)C(=O)N[C@@H](CCCCN)C(N)=O]

MS: Calc'd for C₃₆H₆₁N₁₁O₆S: 775.45, found [M+2H]²⁺: 389.0.

### Example 32 (Compound 32) (SEQ ID NO: 192)

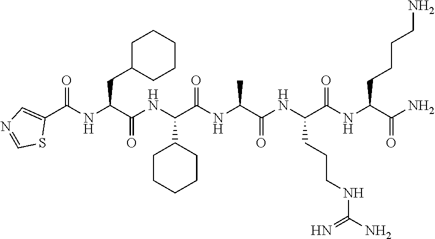 [SMILES: C[C@H](NC(=O)[C@@H](NC(=O)[C@H](CC1CCCCC1)NC(=O)c1cncs1)C1CCCCC1)C(=O)N[C@@H](CCCNC(=N)N)C(=O)N[C@@H](CCCCN)C(N)=O]

MS: Calc'd for C₃₆H₆₁N₁₁O₆S: 775.45, found [M+2H]²⁺: 389.1.

### Example 33 (Compound 33) (SEQ ID NO: 193)

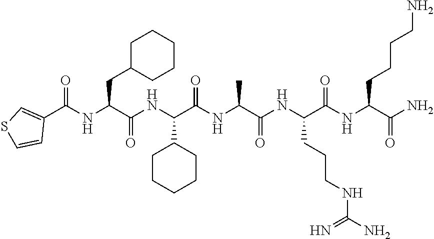 [SMILES: C[C@H](NC(=O)[C@@H](NC(=O)[C@H](CC1CCCCC1)NC(=O)c1ccsc1)C1CCCCC1)C(=O)N[C@@H](CCCNC(=N)N)C(=O)N[C@@H](CCCCN)C(N)=O]

MS: Calc'd for C₃₇H₆₂N₁₀O₆S: 774.46, found [M+2H]²⁺: 388.5.

### Example 34 (Compound 34) (SEQ ID NO: 194)

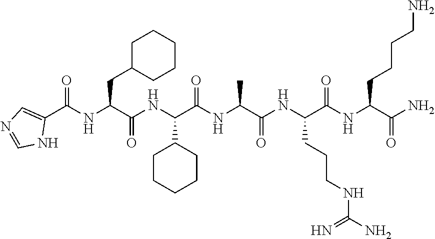 [SMILES: C[C@H](NC(=O)[C@@H](NC(=O)[C@H](CC1CCCCC1)NC(=O)c1cnc[nH]1)C1CCCCC1)C(=O)N[C@@H](CCCNC(=N)N)C(=O)N[C@@H](CCCCN)C(N)=O]

MS: Calc'd for C₃₆H₆₂N₁₂O₆: 758.49, found [M+2H]²⁺: 380.5.

### Example 35 (Compound 35) (SEQ ID NO: 195)

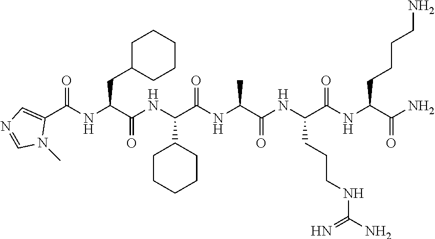 [SMILES: C[C@H](NC(=O)[C@@H](NC(=O)[C@H](CC1CCCCC1)NC(=O)c1cncn1C)C1CCCCC1)C(=O)N[C@@H](CCCNC(=N)N)C(=O)N[C@@H](CCCCN)C(N)=O]

MS: Calc'd for C₃₇H₆₄N₁₂O₆: 772.51, found [M+2H]²⁺: 387.5.

### Example 36 (Compound 36) (SEQ ID NO: 196)

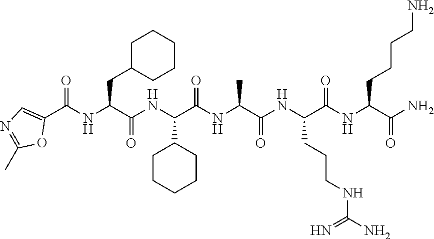 [SMILES: Cc1ncc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)o1]

MS: Calc'd for C₃₇H₆₃N₁₁O₇: 773.49, found [M+2H]²⁺: 388.0.

### Example 37 (Compound 37) (SEQ ID NO: 197)

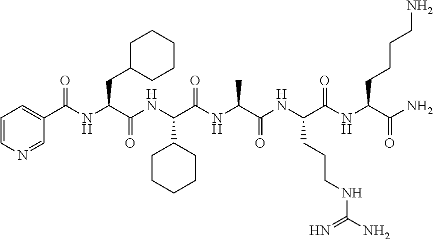 [SMILES: C[C@H](NC(=O)[C@@H](NC(=O)[C@H](CC1CCCCC1)NC(=O)c1cccnc1)C1CCCCC1)C(=O)N[C@@H](CCCNC(=N)N)C(=O)N[C@@H](CCCCN)C(N)=O]

MS: Calc'd for C₃₈H₆₃N₁₁O₆: 769.50, found [M+2H]²⁺: 386.1.

### Example 38 (Compound 38) (SEQ ID NO: 198)

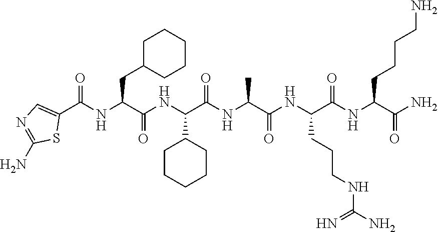 [SMILES: C[C@H](NC(=O)[C@@H](NC(=O)[C@H](CC1CCCCC1)NC(=O)c1cnc(N)s1)C1CCCCC1)C(=O)N[C@@H](CCCNC(=N)N)C(=O)N[C@@H](CCCCN)C(N)=O]

MS: Calc'd for C₃₆H₆₂N₁₂O₆S: 790.46, found [M+2H]²⁺: 396.5.

### Example 39 (Compound 39) (SEQ ID NO: 199)

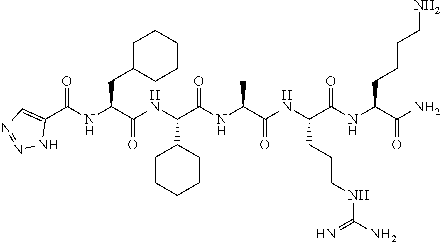 [SMILES: C[C@H](NC(=O)[C@@H](NC(=O)[C@H](CC1CCCCC1)NC(=O)c1cnn[nH]1)C1CCCCC1)C(=O)N[C@@H](CCCNC(=N)N)C(=O)N[C@@H](CCCCN)C(N)=O]

MS: Calc'd for C₃₅H₆₁N₁₃O₆: 759.49, found [M+2H]²⁺: 381.0.

### Example 40 (Compound 40) (SEQ ID NO: 200)

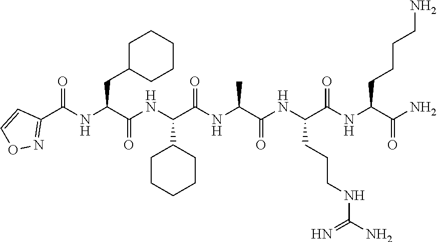 [SMILES: C[C@H](NC(=O)[C@@H](NC(=O)[C@H](CC1CCCCC1)NC(=O)c1ccon1)C1CCCCC1)C(=O)N[C@@H](CCCNC(=N)N)C(=O)N[C@@H](CCCCN)C(N)=O]

MS: Calc'd for C₃₆H₆₁N₁₁O₇: 759.48, found [M+2H]²⁺: 381.0.

### Example 41 (Compound 41) (SEQ ID NO: 201)

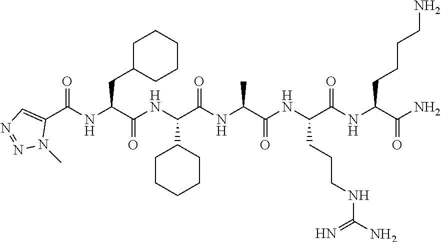 [SMILES: C[C@H](NC(=O)[C@@H](NC(=O)[C@H](CC1CCCCC1)NC(=O)c1cnnn1C)C1CCCCC1)C(=O)N[C@@H](CCCNC(=N)N)C(=O)N[C@@H](CCCCN)C(N)=O]

MS: Calc'd for C₃₆H₆₃N₁₃O₆: 773.50, found [M+2H]²⁺: 388.0.

### Example 42 (Compound 42) (SEQ ID NO: 202)

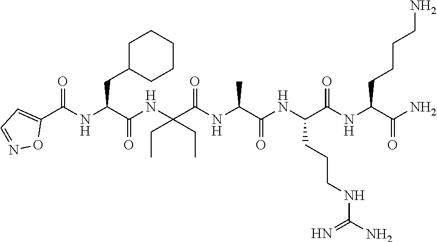 [SMILES: CCC(CC)(NC(=O)[C@H](CC1CCCCC1)NC(=O)c1ccno1)C(=O)N[C@@H](C)C(=O)N[C@@H](CCCNC(=N)N)C(=O)N[C@@H](CCCCN)C(N)=O]

### Example 43 (Compound 43) (SEQ ID NO: 203)

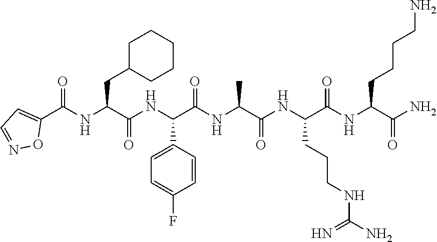 [SMILES: C[C@H](NC(=O)[C@@H](NC(=O)[C@H](CC1CCCCC1)NC(=O)c1ccno1)c1ccc(F)cc1)C(=O)N[C@@H](CCCNC(=N)N)C(=O)N[C@@H](CCCCN)C(N)=O]

### Example 44 (Compound 44) (SEQ ID NO: 204)

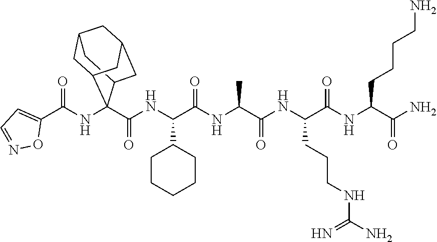 [SMILES: C[C@H](NC(=O)[C@@H](NC(=O)C1(NC(=O)c2ccno2)C2CC3CC(C2)CC1C3)C1CCCCC1)C(=O)N[C@@H](CCCNC(=N)N)C(=O)N[C@@H](CCCCN)C(N)=O]

### Example 45 (Compound 45) (SEQ ID NO: 205)

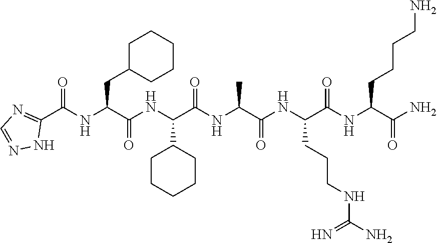 [SMILES: C[C@H](NC(=O)[C@@H](NC(=O)[C@H](CC1CCCCC1)NC(=O)c1ncn[nH]1)C1CCCCC1)C(=O)N[C@@H](CCCNC(=N)N)C(=O)N[C@@H](CCCCN)C(N)=O]

### Example 46 (Compound 46) (SEQ ID NO: 206)

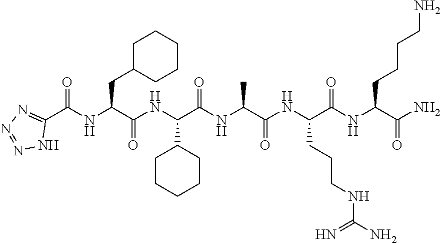 [SMILES: C[C@H](NC(=O)[C@@H](NC(=O)[C@H](CC1CCCCC1)NC(=O)c1nnn[nH]1)C1CCCCC1)C(=O)N[C@@H](CCCNC(=N)N)C(=O)N[C@@H](CCCCN)C(N)=O]

### Example 47 (Compound 47) (SEQ ID NO: 207)

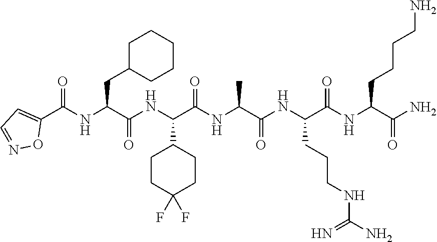 [SMILES: C[C@H](NC(=O)[C@@H](NC(=O)[C@H](CC1CCCCC1)NC(=O)c1ccno1)C1CCC(F)(F)CC1)C(=O)N[C@@H](CCCNC(=N)N)C(=O)N[C@@H](CCCCN)C(N)=O]

### Example 48 (Compound 48)

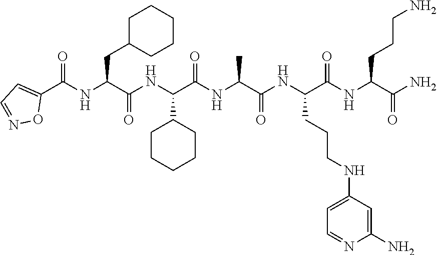 [SMILES: C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N[C@@H](CCCN)C(N)=O]

MS: Calc'd for C₃₉H₆₁N₁₁O₇: 795.48, found [M+H]⁺: 796.3.

### Example 49 (Compound 49)

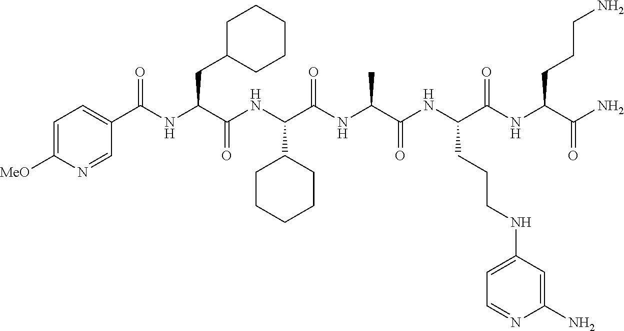 [SMILES: COc1ccc(C(=O)N[C@@H](CC2CCCCC2)C(=O)N[C@H](C(=O)N[C@@H](C)C(=O)N[C@@H](CCCNc2ccnc(N)c2)C(=O)N[C@@H](CCCN)C(N)=O)C2CCCCC2)cn1]

MS: Calc'd for C₄₂H₆₅N₁₁O₇: 835.51, found [M+H]⁺: 836.4.

### Example 50 (Compound 50)

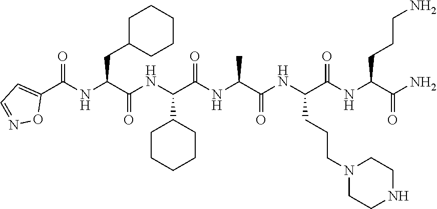 [SMILES: C[C@H](NC(=O)[C@@H](NC(=O)[C@H](CC1CCCCC1)NC(=O)c1ccno1)C1CCCCC1)C(=O)N[C@@H](CCCN1CCNCC1)C(=O)N[C@@H](CCCN)C(N)=O]

### Synthesis of(S)-2-((((9H-fluoren-9-yl) methoxy) carbonyl)amino)-5-(4-(tert-butoxycarbonyl) piperazin-1-yl) pentanoic acid

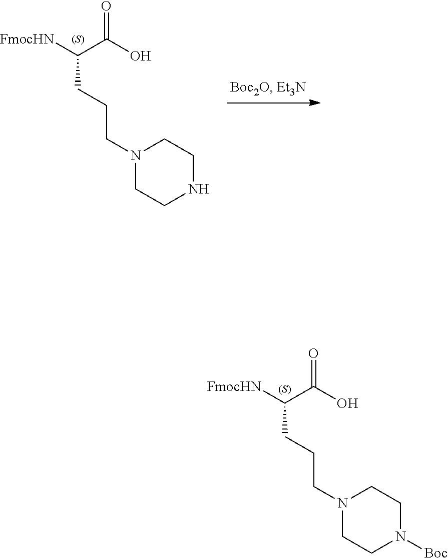

A mixture of(S)-2-((((9H-fluoren-9-yl) methoxy) carbonyl)amino)-5-(piperazin-1-yl) pentanoic acid (450 mg, 75% Wt, 1 Eq, 797 μmol), Boc₂O (350 mg, 368 μL, 2.01 Eq, 1.60 mmol), TEA (161 mg, 222 μL, 2.00 Eq, 1.59 mmol) and DCM (5 mL) was stirred at 25° C. for 1 hour. The mixture was directly purified by MPLC to provide(S)-2-((((9H-fluoren-9-yl) methoxy) carbonyl)amino)-5-(4-(tert-butoxycarbonyl) piperazin-1-yl) pentanoic acid (300 mg, 573 μmol, 71.9%) as a white solid. MS: Calc'd for C₂₉H₃₇N₃O₆: 523.27, found [M+H]⁺: 524.2.

Compound 50 was synthesized according to the generic procedures described above to provide the product. MS: Calc'd for C₃₈H₆₃N₁₀O₇: 772.50, found [M+H]⁺: 773.4.

### Example 51 (Compound 51)

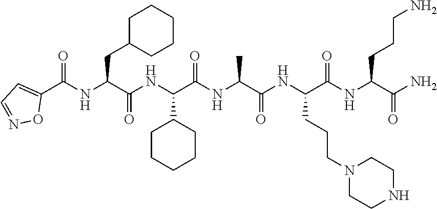 [SMILES: C[C@H](NC(=O)[C@@H](NC(=O)[C@H](CC1CCCCC1)NC(=O)c1ccno1)C1CCCCC1)C(=O)N[C@@H](CCCN1CCNCC1)C(=O)N[C@@H](CCCN)C(N)=O]

MS: Calc'd for C₃₉H₆₅N₉O₇: 771.50, found [M+H]⁺: 772.5.

### Example 52 (Compound 52)

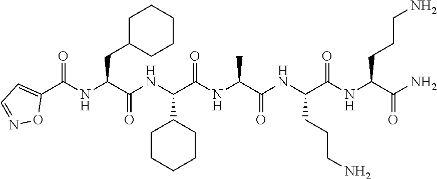 [SMILES: C[C@H](NC(=O)[C@@H](NC(=O)[C@H](CC1CCCCC1)NC(=O)c1ccno1)C1CCCCC1)C(=O)N[C@@H](CCCN)C(=O)N[C@@H](CCCN)C(N)=O]

MS: Calc'd for C₃₄H₅₇N₉O₇: 703.44, found [M+H]⁺: 704.3.

### Example 53 (Compound 53) (SEQ ID NO: 213)

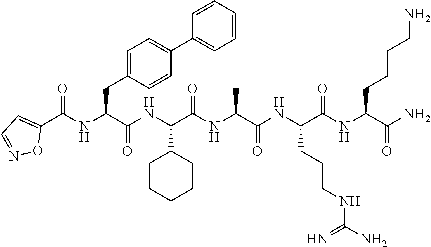 [SMILES: C[C@H](NC(=O)[C@@H](NC(=O)[C@H](Cc1ccc(-c2ccccc2)cc1)NC(=O)c1ccno1)C1CCCCC1)C(=O)N[C@@H](CCCNC(=N)N)C(=O)N[C@@H](CCCCN)C(N)=O]

MS: Calc'd for C₄₂H₅N₁₁O₇: 829.46, found [M+H]⁺: 830.3.

### Example 54 (Compound 54) (SEQ ID NO: 214)

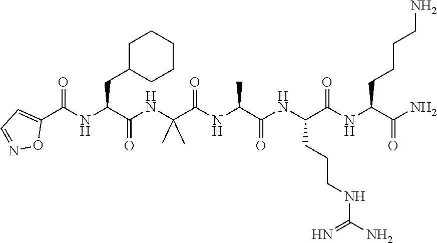 [SMILES: C[C@H](NC(=O)C(C)(C)NC(=O)[C@H](CC1CCCCC1)NC(=O)c1ccno1)C(=O)N[C@@H](CCCNC(=N)N)C(=O)N[C@@H](CCCCN)C(N)=O]

MS: Calc'd for C₃₂H₅₅N₁₁O₇: 705.43, found [M+H]⁺: 706.3.

### Example 55 (Compound 55) (SEQ ID NO: 215)

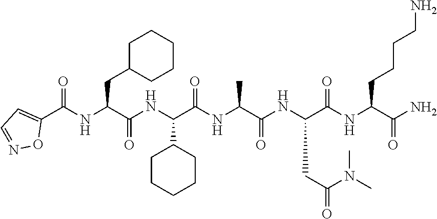 [SMILES: C[C@H](NC(=O)[C@@H](NC(=O)[C@H](CC1CCCCC1)NC(=O)c1ccno1)C1CCCCC1)C(=O)N[C@@H](CC(=O)N(C)C)C(=O)N[C@@H](CCCCN)C(N)=O]

MS: Calc'd for C₃₆H₅₉N₉O₈: 745.45, found [M+H]⁺: 746.4.

### Example 56 (Compound 56) (SEQ ID NO: 216)

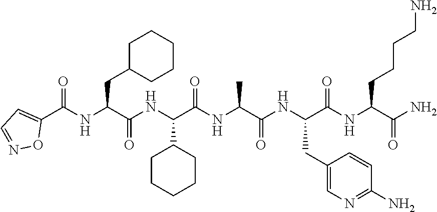 [SMILES: C[C@H](NC(=O)[C@@H](NC(=O)[C@H](CC1CCCCC1)NC(=O)c1ccno1)C1CCCCC1)C(=O)N[C@@H](Cc1ccc(N)nc1)C(=O)N[C@@H](CCCCN)C(N)=O]

MS: Calc'd for C₃₈H₅₈N₁₀O₇: 766.45, found [M+H]⁺: 767.4.

### Example 57 (Compound 57) (SEQ ID NO: 217)

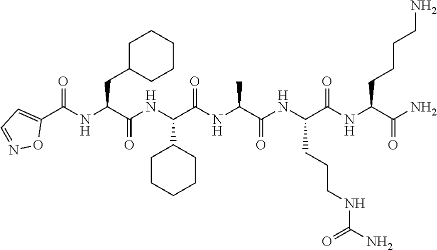 [SMILES: C[C@H](NC(=O)[C@@H](NC(=O)[C@H](CC1CCCCC1)NC(=O)c1ccno1)C1CCCCC1)C(=O)N[C@@H](CCCNC(N)=O)C(=O)N[C@@H](CCCCN)C(N)=O]

MS: Calc'd for C₃₆H₆₀N₁₀O₈: 760.46, found [M+H]⁺: 761.4.

### Example 58 (Compound 58) (SEQ ID NO: 218)

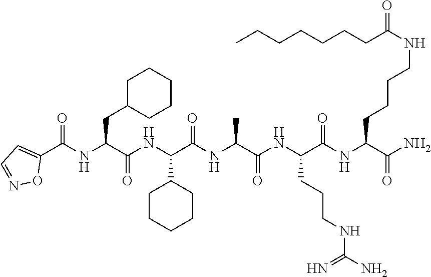 [SMILES: CCCCCCCC(=O)NCCCC[C@H](NC(=O)[C@H](CCCNC(=N)N)NC(=O)[C@H](C)NC(=O)[C@@H](NC(=O)[C@H](CC1CCCCC1)NC(=O)c1ccno1)C1CCCCC1)C(N)=O]

MS: Calc'd for C₄₄H₇₅N₁₁O₈: 885.58, found [M+H]⁺: 886.5.

### Example 59 (Compound 59) (SEQ ID NO: 219)

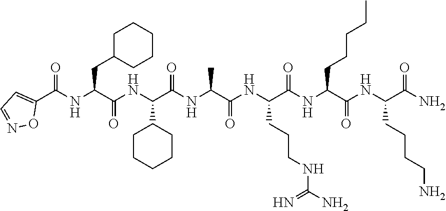 [SMILES: CCCCC[C@H](NC(=O)[C@H](CCCNC(=N)N)NC(=O)[C@H](C)NC(=O)[C@@H](NC(=O)[C@H](CC1CCCCC1)NC(=O)c1ccno1)C1CCCCC1)C(=O)N[C@@H](CCCCN)C(N)=O]

MS: Calc'd for C₄₃H₇₄N₁₂O₈: 886.58, found [M+H]⁺: 887.5.

### Example 60 (Compound 60) (SEQ ID NO: 220)

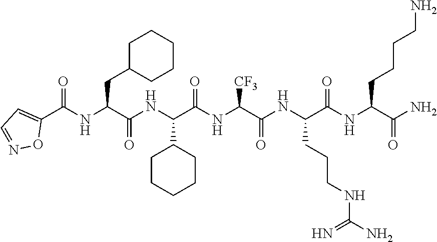 [SMILES: N=C(N)NCCC[C@H](NC(=O)[C@@H](NC(=O)[C@@H](NC(=O)[C@H](CC1CCCCC1)NC(=O)c1ccno1)C1CCCCC1)C(F)(F)F)C(=O)N[C@@H](CCCCN)C(N)=O]

### Example 61 (Compound 61) (SEQ ID NO: 221)

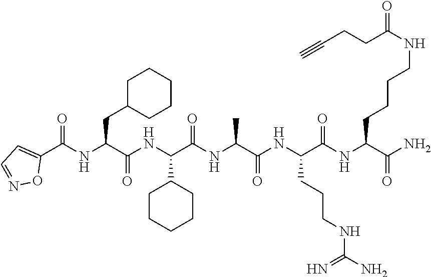 [SMILES: C#CCCC(=O)NCCCC[C@H](NC(=O)[C@H](CCCNC(=N)N)NC(=O)[C@H](C)NC(=O)[C@@H](NC(=O)[C@H](CC1CCCCC1)NC(=O)c1ccno1)C1CCCCC1)C(N)=O]

MS: Calc'd for C₄₁H₆₅N₁₁O₈: 839.50, found [M+2H]²⁺: 421.0.

### Example 62 (Compound 62) (SEQ ID NO: 222)

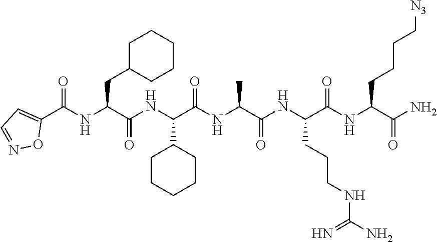 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCCN=[N+]=[N-])C(N)=O]

MS: Calc'd for C₃₆H₅₉N₁₃O₇: 785.47, found [M+H]⁺: 785.5.

### Example 63 (Compound 63) (SEQ ID NO: 223)

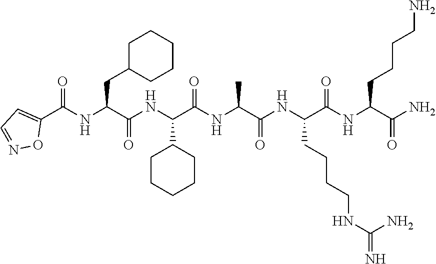 [SMILES: C[C@H](NC(=O)[C@@H](NC(=O)[C@H](CC1CCCCC1)NC(=O)c1ccno1)C1CCCCC1)C(=O)N[C@@H](CCCCNC(=N)N)C(=O)N[C@@H](CCCCN)C(N)=O]

MS: Calc'd for C₃₇H₆₃N₁₁O₇: 773.49, found [M+H]⁺: 774.4.

### Example 64 (Compound 64) (SEQ ID NO: 224)

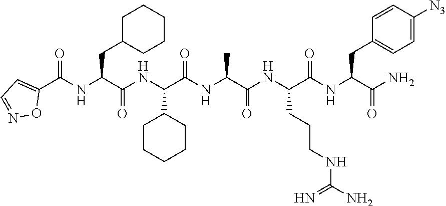 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](Cc1ccc(N=[N+]=[N-])cc1)C(N)=O]

### Example 65 (Compound 65) (SEQ ID NO: 225)

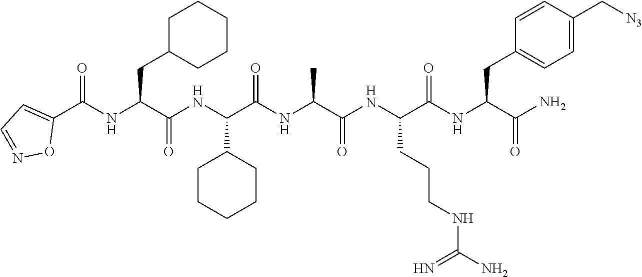 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](Cc1ccc(CN=[N+]=[N-])cc1)C(N)=O]

### Example 66 (Compound 66) (SEQ ID NO: 226)

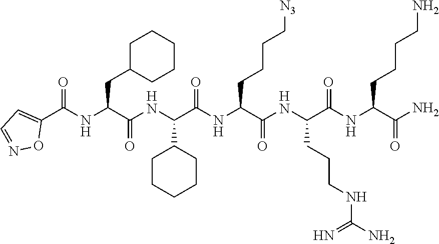 [SMILES: [N-]=[N+]=NCCCC[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCCN)C(N)=O]

MS: Calc'd for C₃₅H₆₆N₁₄O₇: 842.52, found [M+2H]²⁺: 422.6.

### Example 67 (Compound 67) (SEQ ID NO: 227)

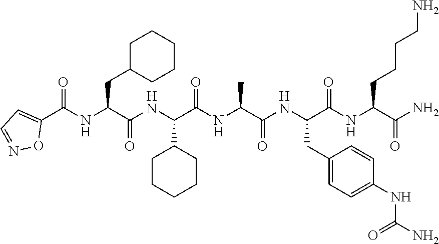 [SMILES: C[C@H](NC(=O)[C@@H](NC(=O)[C@H](CC1CCCCC1)NC(=O)c1ccno1)C1CCCCC1)C(=O)N[C@@H](Cc1ccc(NC(N)=O)cc1)C(=O)N[C@@H](CCCCN)C(N)=O]

MS: Calc'd for C₄₀H₆₀N10O₈: 808.46, found [M+H]⁺: 809.6.

### Example 68 (Compound 68) (SEQ ID NO: 228)

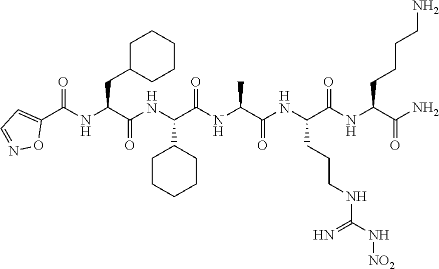 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N[N+](=O)[O-])C(=O)N[C@@H](CCCCN)C(N)=O]

MS: Calc'd for C₃₆H₆₀N₁₂O₉: 804.46, found [M+H]⁺: 805.6.

### Example 69 (Compound 69) (SEQ ID NO: 229)

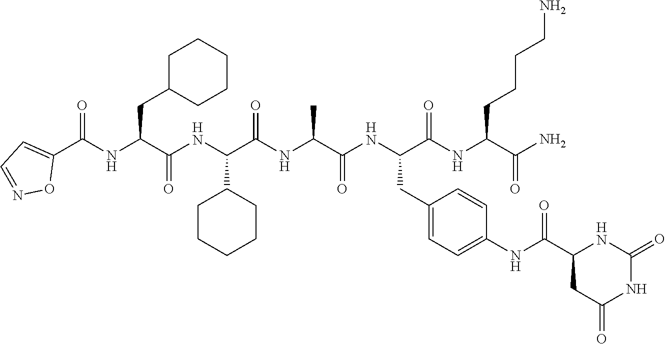 [SMILES: C[C@H](NC(=O)[C@@H](NC(=O)[C@H](CC1CCCCC1)NC(=O)c1ccno1)C1CCCCC1)C(=O)N[C@@H](Cc1ccc(NC(=O)[C@@H]2CC(=O)NC(=O)N2)cc1)C(=O)N[C@@H](CCCCN)C(N)=O]

MS: Calc'd for C₄₄H₆₃N₁₁O₁₀: 905.48, found [M+H]⁺: 906.7.

### Example 70 (Compound 70) (SEQ ID NO: 230)

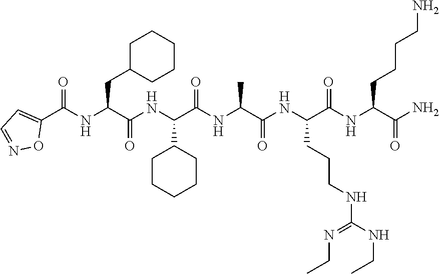 [SMILES: CC/N=C(\NCC)NCCC[C@H](NC(=O)[C@H](C)NC(=O)[C@@H](NC(=O)[C@H](CC1CCCCC1)NC(=O)c1ccno1)C1CCCCC1)C(=O)N[C@@H](CCCCN)C(N)=O]

### Example 71 (Compound 71) (SEQ ID NO: 231)

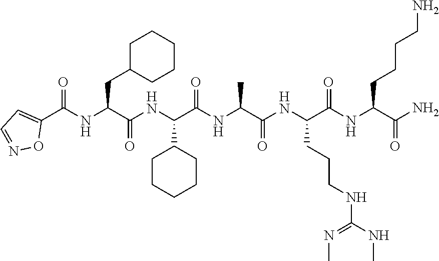 [SMILES: C/N=C(\NC)NCCC[C@H](NC(=O)[C@H](C)NC(=O)[C@@H](NC(=O)[C@H](CC1CCCCC1)NC(=O)c1ccno1)C1CCCCC1)C(=O)N[C@@H](CCCCN)C(N)=O]

### Example 72 (Compound 72) (SEQ ID NO: 232)

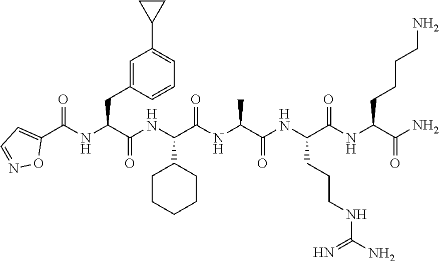 [SMILES: C[C@H](NC(=O)[C@@H](NC(=O)[C@H](Cc1cccc(C2CC2)c1)NC(=O)c1ccno1)C1CCCCC1)C(=O)N[C@@H](CCCNC(=N)N)C(=O)N[C@@H](CCCCN)C(N)=O]

MS: Calc'd for C₃₉H₅₉N₁₁O₇: 793.46, found [M+H]⁺: 794.4.

### Example 73 (Compound 73) (SEQ ID NO: 233)

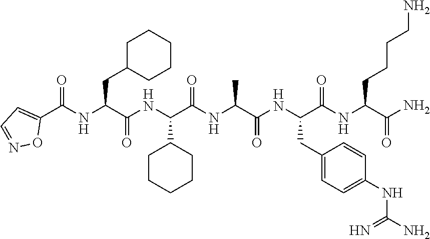 [SMILES: C[C@H](NC(=O)[C@@H](NC(=O)[C@H](CC1CCCCC1)NC(=O)c1ccno1)C1CCCCC1)C(=O)N[C@@H](Cc1ccc(NC(=N)N)cc1)C(=O)N[C@@H](CCCCN)C(N)=O]

### Example 74 (Compound 74) (SEQ ID NO: 234)

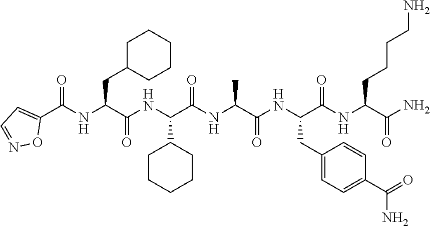 [SMILES: C[C@H](NC(=O)[C@@H](NC(=O)[C@H](CC1CCCCC1)NC(=O)c1ccno1)C1CCCCC1)C(=O)N[C@@H](Cc1ccc(C(N)=O)cc1)C(=O)N[C@@H](CCCCN)C(N)=O]

MS: Calc'd for C₄₀H₅,N₉O₈: 793.45, found [M+H]⁺: 794.5.

### Example 75 (Compound 75) (SEQ ID NO: 235)

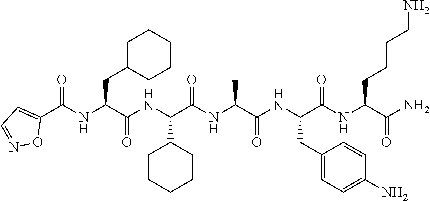 [SMILES: C[C@H](NC(=O)[C@@H](NC(=O)[C@H](CC1CCCCC1)NC(=O)c1ccno1)C1CCCCC1)C(=O)N[C@@H](Cc1ccc(N)cc1)C(=O)N[C@@H](CCCCN)C(N)=O]

MS: Calc'd for C₃₉H₅₉N₉O₇: 765.45, found [M+2H]²⁺: 384.0.

### Example 76 (Compound 76) (SEQ ID NO: 236)

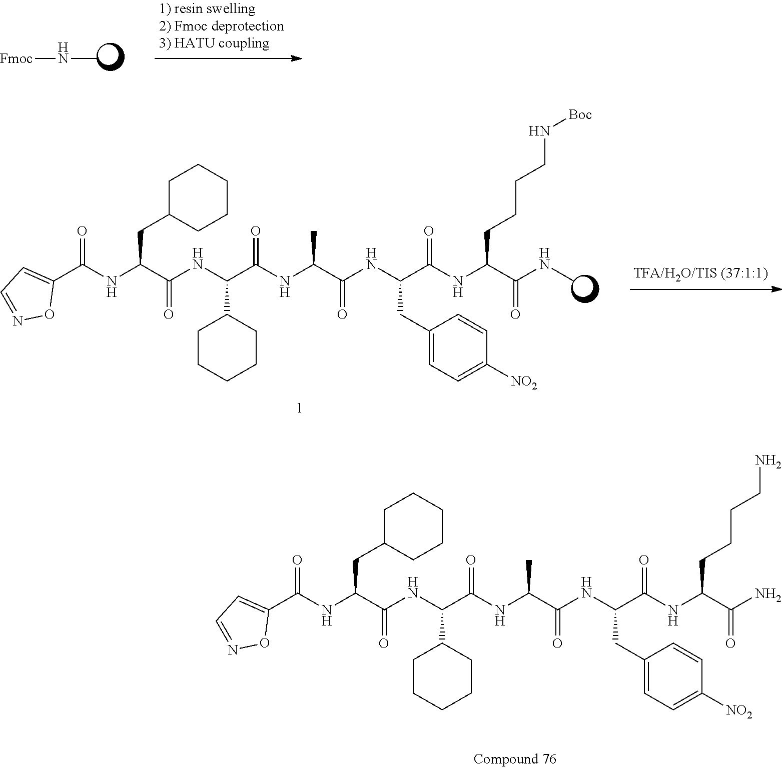

Peptidyl resin 1 was prepared by standard Fmoc-based SPPS procedures from Na-Fmoc-Sieber amide resin (0.30 g, 0.71 mmol/g, 0.21 mmol) (General SPPS procedure 1-3).

Peptidyl resin 1 was treated with a mixture of TFA/H₂O/TIS (37:1:1 v/v/v, 10 mL) at room temperature. After 1 h, the mixture was filtered, and the filtrate was concentrated under reduced pressure. The residue was purified by RP-HPLC (Mobile Phase A: water (0.05% TFA), Mobile Phase B: acetonitrile). The desired fractions were combined, concentrated and lyophilized to afford Compound 76 (41 mg, 21.1% overall yield, 97.5% purity) as a white solid. MS: Calc'd for C₃₉H₅₇N₉O₉: 795.43, found [M+H]⁺: 796.6.

### Example 77 (Compound 77) (SEQ ID NO: 237)

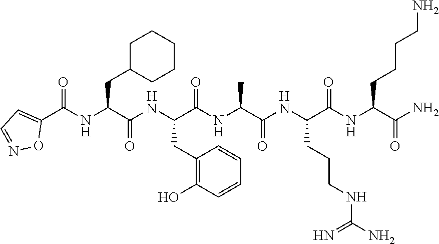 [SMILES: C[C@H](NC(=O)[C@H](Cc1ccccc1O)NC(=O)[C@H](CC1CCCCC1)NC(=O)c1ccno1)C(=O)N[C@@H](CCCNC(=N)N)C(=O)N[C@@H](CCCCN)C(N)=O]

MS: Calc'd for C₃₇H₅₇N₁₁O₈: 783.44, found [M+2H]²⁺: 393.

### Example 78 (Compound 78) (SEQ ID NO: 238)

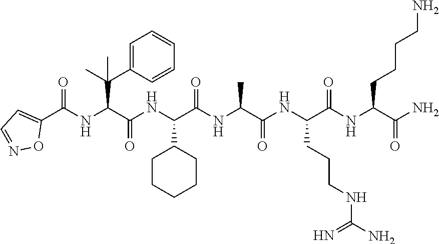 [SMILES: C[C@H](NC(=O)[C@@H](NC(=O)[C@@H](NC(=O)c1ccno1)C(C)(C)c1ccccc1)C1CCCCC1)C(=O)N[C@@H](CCCNC(=N)N)C(=O)N[C@@H](CCCCN)C(N)=O]

MS: Calc'd for C₃₈H₅N₁₁O₇: 781.46, found [M+2H]²⁺: 391.9.

### Example 79 (Compound 79) (SEQ ID NO: 239)

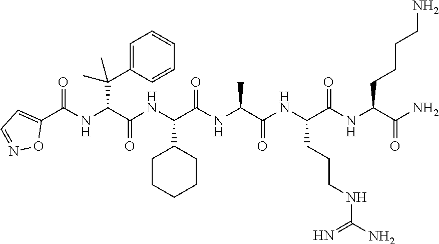 [SMILES: C[C@H](NC(=O)[C@@H](NC(=O)[C@H](NC(=O)c1ccno1)C(C)(C)c1ccccc1)C1CCCCC1)C(=O)N[C@@H](CCCNC(=N)N)C(=O)N[C@@H](CCCCN)C(N)=O]

MS: Calc'd for C₃₈H₅N₁₁O₇: 781.46, found [M+2H]²⁺: 392.0.

### Example 80 (Compound 80) (SEQ ID NO: 240)

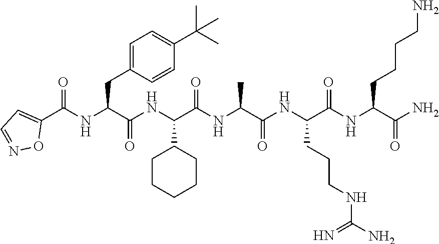 [SMILES: C[C@H](NC(=O)[C@@H](NC(=O)[C@H](Cc1ccc(C(C)(C)C)cc1)NC(=O)c1ccno1)C1CCCCC1)C(=O)N[C@@H](CCCNC(=N)N)C(=O)N[C@@H](CCCCN)C(N)=O]

### Example 81 (Compound 81) (SEQ ID NO: 241)

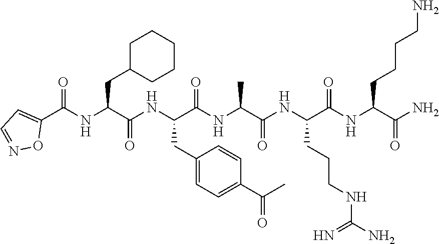 [SMILES: CC(=O)c1ccc(C[C@H](NC(=O)[C@H](CC2CCCCC2)NC(=O)c2ccno2)C(=O)N[C@@H](C)C(=O)N[C@@H](CCCNC(=N)N)C(=O)N[C@@H](CCCCN)C(N)=O)cc1]

### Example 82 (Compound 82) (SEQ ID NO: 242)

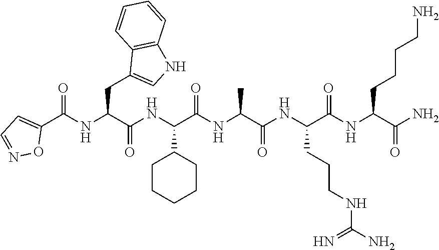 [SMILES: C[C@H](NC(=O)[C@@H](NC(=O)[C@H](Cc1c[nH]c2ccccc12)NC(=O)c1ccno1)C1CCCCC1)C(=O)N[C@@H](CCCNC(=N)N)C(=O)N[C@@H](CCCCN)C(N)=O]

### Example 83 (Compound 83) (SEQ ID NO: 243)

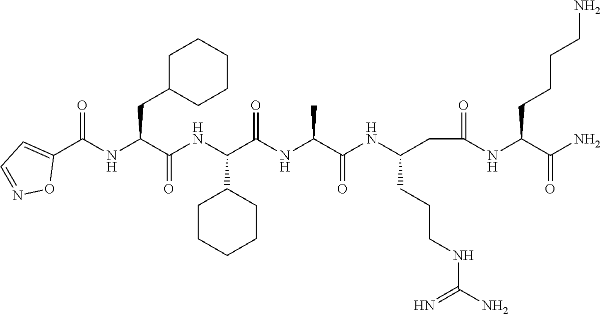 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)CC(=O)N[C@@H](CCCCN)C(N)=O]

### Example 84 (Compound 84) (SEQ ID NO: 244)

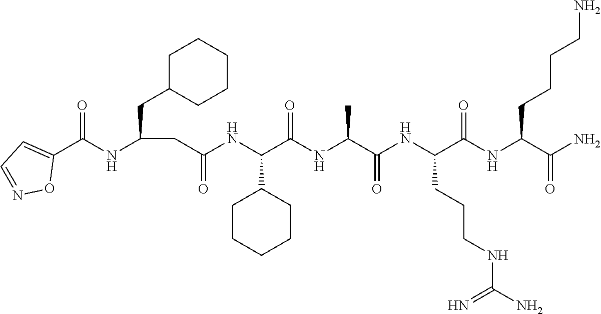 [SMILES: C[C@H](NC(=O)[C@@H](NC(=O)C[C@H](CC1CCCCC1)NC(=O)c1ccno1)C1CCCCC1)C(=O)N[C@@H](CCCNC(=N)N)C(=O)N[C@@H](CCCCN)C(N)=O]

### Example 85 (Compound 85) (SEQ ID NO: 245)

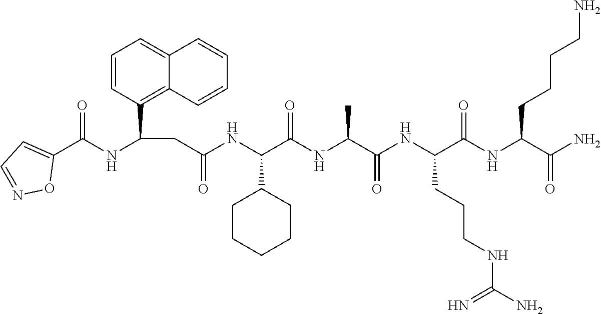 [SMILES: C[C@H](NC(=O)[C@@H](NC(=O)C[C@@H](NC(=O)c1ccno1)c1cccc2ccccc12)C1CCCCC1)C(=O)N[C@@H](CCCNC(=N)N)C(=O)N[C@@H](CCCCN)C(N)=O]

MS: Calc'd for C₄₀H₅₇N₁₁O₇: 803.44, found [M+2H]²⁺: 403.0.

### Example 86 (Compound 86) (SEQ ID NO: 246)

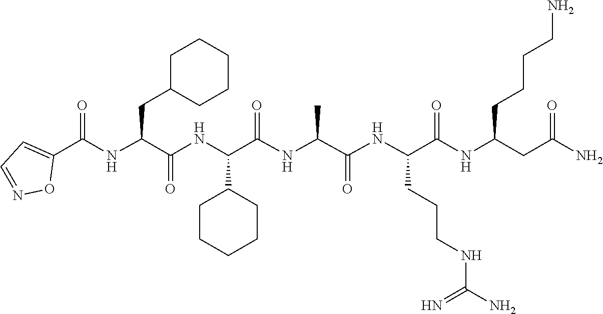 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCCN)CC(N)=O]

MS: Calc'd for C₃₇H₆₃N₁₁O₇: 773.49, found [M+2H]²⁺: 388.1.

### Example 87 (Compound 87) (SEQ ID NO: 247)

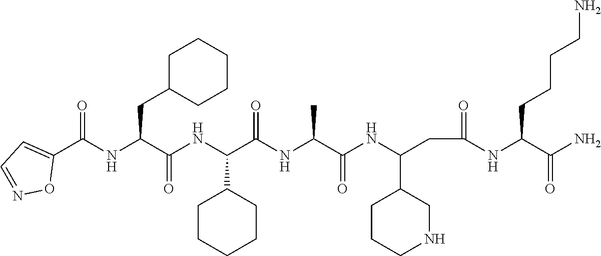 [SMILES: C[C@H](NC(=O)[C@@H](NC(=O)[C@H](CC1CCCCC1)NC(=O)c1ccno1)C1CCCCC1)C(=O)NC(CC(=O)N[C@@H](CCCCN)C(N)=O)C1CCCNC1]

MS: Calc'd for C₃₂H₆₃N₉O₇: 757.49, found [M+2H]²⁺: 380.1.

### Example 88 (Compound 88) (SEQ ID NO: 248)

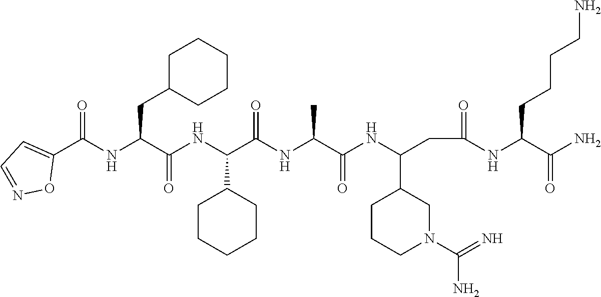 [SMILES: C[C@H](NC(=O)[C@@H](NC(=O)[C@H](CC1CCCCC1)NC(=O)c1ccno1)C1CCCCC1)C(=O)NC(CC(=O)N[C@@H](CCCCN)C(N)=O)C1CCCN(C(=N)N)C1]

### Example 89 (Compound 89) (SEQ ID NO: 249)

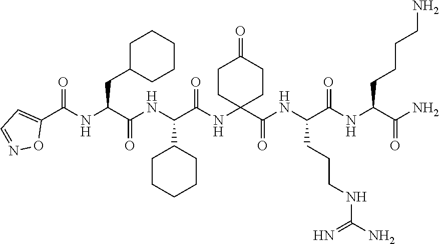 [SMILES: N=C(N)NCCC[C@H](NC(=O)C1(NC(=O)[C@@H](NC(=O)[C@H](CC2CCCCC2)NC(=O)c2ccno2)C2CCCCC2)CCC(=O)CC1)C(=O)N[C@@H](CCCCN)C(N)=O]

### Example 90 (Compound 90) (SEQ ID NO: 250)

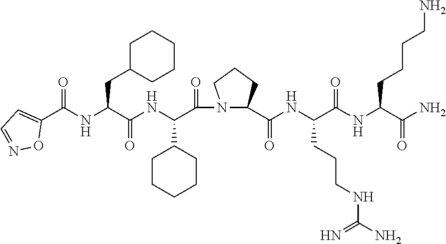 [SMILES: N=C(N)NCCC[C@H](NC(=O)[C@@H]1CCCN1C(=O)[C@@H](NC(=O)[C@H](CC1CCCCC1)NC(=O)c1ccno1)C1CCCCC1)C(=O)N[C@@H](CCCCN)C(N)=O]

### Example 91 (Compound 91) (SEQ ID NO: 251)

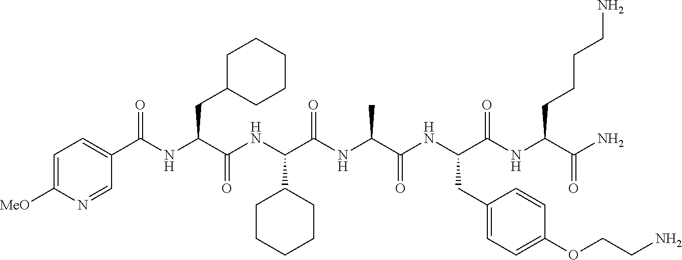 [SMILES: COc1ccc(C(=O)N[C@@H](CC2CCCCC2)C(=O)N[C@H](C(=O)N[C@@H](C)C(=O)N[C@@H](Cc2ccc(OCCN)cc2)C(=O)N[C@@H](CCCCN)C(N)=O)C2CCCCC2)cn1]

MS: Calc'd for C₄₄H₆₇N₉O₈: 849.51, found [M+2H]²⁺: 850.4.

### Example 92 (Compound 92) (SEQ ID NO: 252)

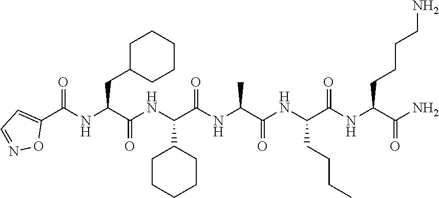 [SMILES: CCCC[C@H](NC(=O)[C@H](C)NC(=O)[C@@H](NC(=O)[C@H](CC1CCCCC1)NC(=O)c1ccno1)C1CCCCC1)C(=O)N[C@@H](CCCCN)C(N)=O]

MS: Calc'd for C₃₆H₆₀N₂O₇: 716.46, found [M+H]⁺: 717.6.

### Example 93 (Compound 93) (SEQ ID NO: 253)

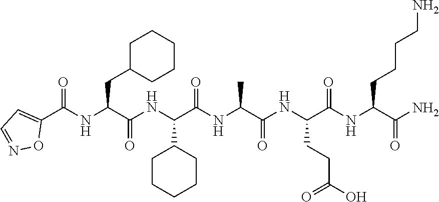 [SMILES: C[C@H](NC(=O)[C@@H](NC(=O)[C@H](CC1CCCCC1)NC(=O)c1ccno1)C1CCCCC1)C(=O)N[C@@H](CCC(=O)O)C(=O)N[C@@H](CCCCN)C(N)=O]

MS: Calc'd for C₃₅H₅₆N₈O₉: 732.42, found [M+H]⁺: 733.6.

### Example 94 (Compound 94) (SEQ ID NO: 254)

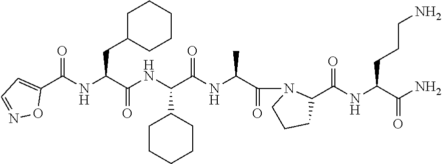

MS: Calc'd for C₃₅H₅₆N₈O₇: 700.43, found [M+H]⁺: 701.6.

### Example 95 (Compound 95) (SEQ ID NO: 255)

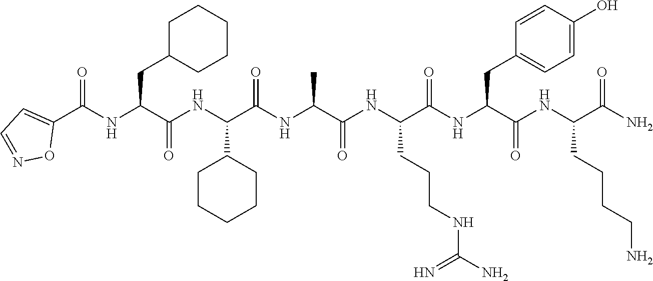 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](Cc1ccc(O)cc1)C(=O)N[C@@H](CCCCN)C(N)=O]

MS: Calc'd for C₄₅H₇N₁₂O₉: 922.54, found [M+2H]²⁺: 462.6.

### Example 96 (Compound 96) (SEQ ID NO: 256)

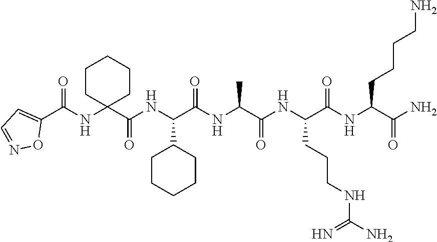 [SMILES: C[C@H](NC(=O)[C@@H](NC(=O)C1(NC(=O)c2ccno2)CCCCC1)C1CCCCC1)C(=O)N[C@@H](CCCNC(=N)N)C(=O)N[C@@H](CCCCN)C(N)=O]

MS: Calc'd for C₃₄H₅₇N₁₁O₇: 731.44, found [M+2H]²⁺: 367.0.

### Example 97 (Compound 97) (SEQ ID NO: 257)

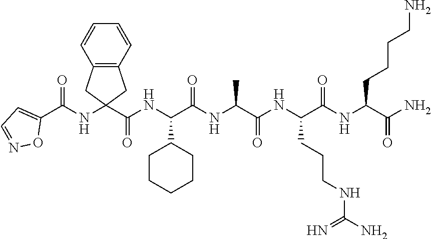 [SMILES: C[C@H](NC(=O)[C@@H](NC(=O)C1(NC(=O)c2ccno2)Cc2ccccc2C1)C1CCCCC1)C(=O)N[C@@H](CCCNC(=N)N)C(=O)N[C@@H](CCCCN)C(N)=O]

MS: Calc'd for C₃₇H₅₅N₁₁O₇: 765.43, found [M+H]⁺: 766.6.

### Example 98 (Compound 98) (SEQ ID NO: 258)

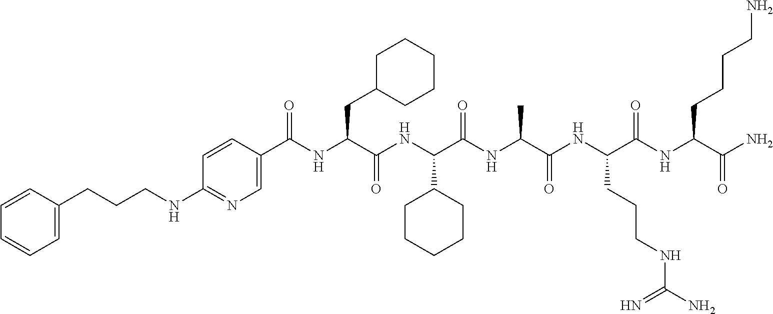 [SMILES: C[C@H](NC(=O)[C@@H](NC(=O)[C@H](CC1CCCCC1)NC(=O)c1ccc(NCCCc2ccccc2)nc1)C1CCCCC1)C(=O)N[C@@H](CCCNC(=N)N)C(=O)N[C@@H](CCCCN)C(N)=O]

MS: Calc'd for C₄₇H₇₄N₁₂O₆: 902.59, found [M+H]⁺: 903.5.

### Example 99 (Compound 99) (SEQ ID NO: 259)

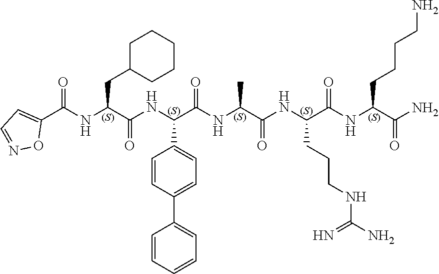

MS: Calc'd for C₄₂H₅₉N₁₁O₇: 829.46, found [M+2H]²⁺: 416.1.

### Example 100 (Compound 100) (SEQ ID NO: 260)

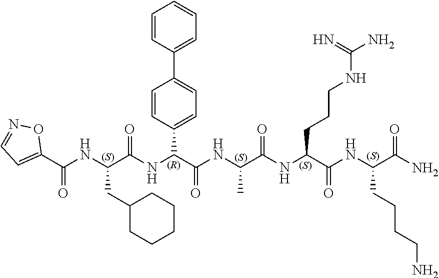

MS: Calc'd for C₄₂H₅₉N₁₁O₇: 829.46, found [M+2H]²⁺: 416.1.

### Example 101 (Compound 101) (SEQ ID NO: 261)

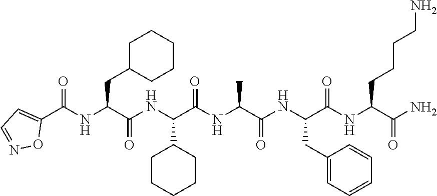 [SMILES: C[C@H](NC(=O)[C@@H](NC(=O)[C@H](CC1CCCCC1)NC(=O)c1ccno1)C1CCCCC1)C(=O)N[C@@H](Cc1ccccc1)C(=O)N[C@@H](CCCCN)C(N)=O]

MS: Calc'd for C₃₉H₅₅N₈O₇: 750.44, found [M+H]⁺: 751.4.

### Example 102 (Compound 102) (SEQ ID NO: 262)

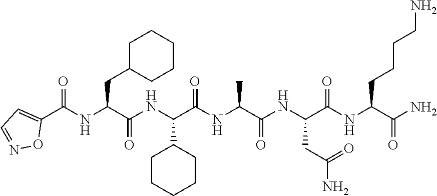 [SMILES: C[C@H](NC(=O)[C@@H](NC(=O)[C@H](CC1CCCCC1)NC(=O)c1ccno1)C1CCCCC1)C(=O)N[C@@H](CC(N)=O)C(=O)N[C@@H](CCCCN)C(N)=O]

MS: Calc'd for C₃₄H₅₅N₉O₈: 717.42, found [M+H]⁺: 718.4.

### Example 103 (Compound 103) (SEQ ID NO: 263)

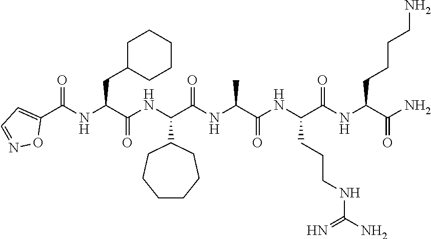 [SMILES: C[C@H](NC(=O)[C@@H](NC(=O)[C@H](CC1CCCCC1)NC(=O)c1ccno1)C1CCCCCC1)C(=O)N[C@@H](CCCNC(=N)N)C(=O)N[C@@H](CCCCN)C(N)=O]

MS: Calc'd for C₃₇H₆₃N₁₁O₇: 773.49, found [M+2H]²⁺: 388.1.

### Example 104 (Compound 104) (SEQ ID NO: 264)

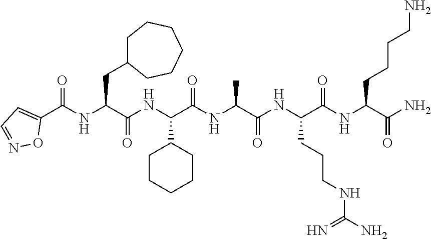 [SMILES: C[C@H](NC(=O)[C@@H](NC(=O)[C@H](CC1CCCCCC1)NC(=O)c1ccno1)C1CCCCC1)C(=O)N[C@@H](CCCNC(=N)N)C(=O)N[C@@H](CCCCN)C(N)=O]

MS: Calc'd for C₃₇H₆₃N₁₁O₇: 773.49, found [M+2H]²⁺: 388.0.

### Example 105 (Compound 105)

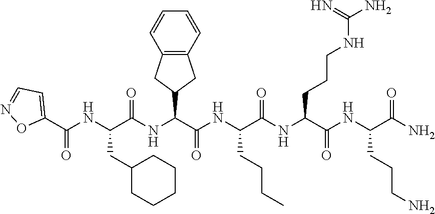 [SMILES: CCCC[C@H](NC(=O)[C@@H](NC(=O)[C@H](CC1CCCCC1)NC(=O)c1ccno1)C1Cc2ccccc2C1)C(=O)N[C@@H](CCCNC(=N)N)C(=O)N[C@@H](CCCN)C(N)=O]

MS: Calc'd for C₄₁H₆₃N₁₁O₇: 821.49, found [M+2H]²⁺: 412.1.

### Example 106 (Compound 106) (SEQ ID NO: 266)

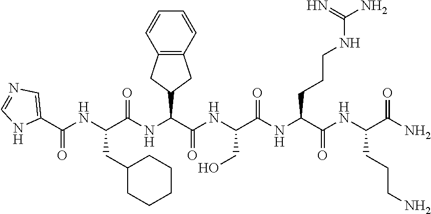 [SMILES: N=C(N)NCCC[C@H](NC(=O)[C@H](CO)NC(=O)[C@@H](NC(=O)[C@H](CC1CCCCC1)NC(=O)c1cnc[nH]1)C1Cc2ccccc2C1)C(=O)N[C@@H](CCCN)C(N)=O]

MS: Calc'd for C₃₈H₅N₁₂O₇: 794.46, found [M+2H]²⁺: 398.5.

### Example 107 (Compound 107) (SEQ ID NO: 267)

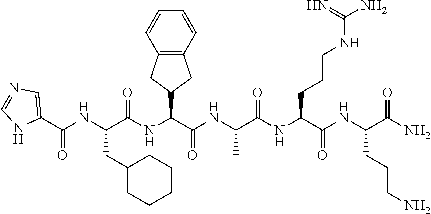 [SMILES: C[C@H](NC(=O)[C@@H](NC(=O)[C@H](CC1CCCCC1)NC(=O)c1cnc[nH]1)C1Cc2ccccc2C1)C(=O)N[C@@H](CCCNC(=N)N)C(=O)N[C@@H](CCCN)C(N)=O]

MS: Calc'd for C₃₈H₅N₁₂O₆: 778.46, found [M+2H]²⁺: 390.5.

### Example 108 (Compound 108) (SEQ ID NO: 268)

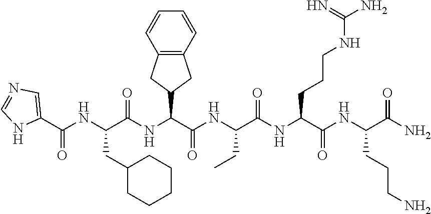 [SMILES: CC[C@H](NC(=O)[C@@H](NC(=O)[C@H](CC1CCCCC1)NC(=O)c1cnc[nH]1)C1Cc2ccccc2C1)C(=O)N[C@@H](CCCNC(=N)N)C(=O)N[C@@H](CCCN)C(N)=O]

MS: Calc'd for C₃₉H₆₀N₁₂O₆: 792.48, found [M+2H]²⁺: 397.5.

### Example 109 (Compound 109) (SEQ ID NO: 269)

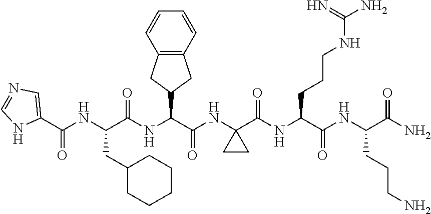 [SMILES: N=C(N)NCCC[C@H](NC(=O)C1(NC(=O)[C@@H](NC(=O)[C@H](CC2CCCCC2)NC(=O)c2cnc[nH]2)C2Cc3ccccc3C2)CC1)C(=O)N[C@@H](CCCN)C(N)=O]

MS: Calc'd for C₃₉H₅₈N₁₂O₆: 790.46, found [M+2H]²⁺: 396.5.

### Example 110 (Compound 110) (SEQ ID NO: 270)

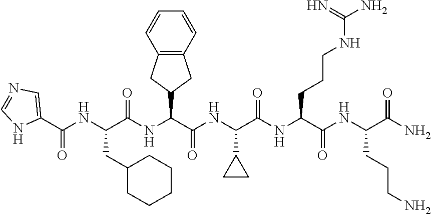 [SMILES: N=C(N)NCCC[C@H](NC(=O)[C@@H](NC(=O)[C@@H](NC(=O)[C@H](CC1CCCCC1)NC(=O)c1cnc[nH]1)C1Cc2ccccc2C1)C1CC1)C(=O)N[C@@H](CCCN)C(N)=O]

MS: Calc'd for C₄₀H₆₀N₁₂O₆: 804.48, found [M+2H]²⁺: 403.5.

### Example 111 (Compound 111) (SEQ ID NO: 271)

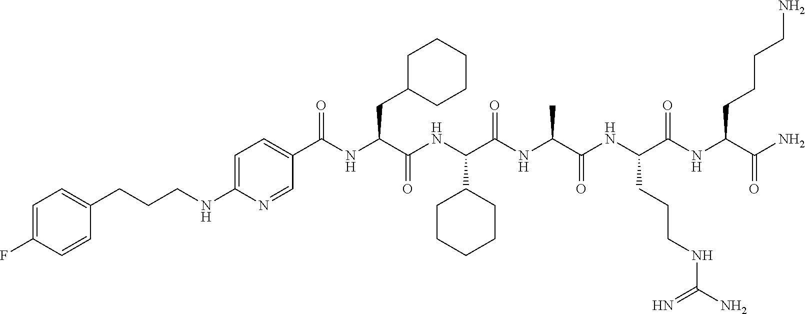 [SMILES: C[C@H](NC(=O)[C@@H](NC(=O)[C@H](CC1CCCCC1)NC(=O)c1ccc(NCCCc2ccc(F)cc2)nc1)C1CCCCC1)C(=O)N[C@@H](CCCNC(=N)N)C(=O)N[C@@H](CCCCN)C(N)=O]

MS: Calc'd for C₄₇H₇₃FN₁₂O₆: 920.58, found [M+H]⁺: 921.5.

### Example 112 (Compound 112) (SEQ ID NO: 272)

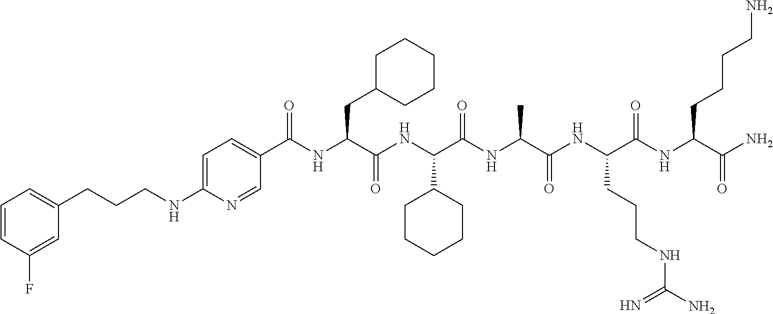 [SMILES: C[C@H](NC(=O)[C@@H](NC(=O)[C@H](CC1CCCCC1)NC(=O)c1ccc(NCCCc2cccc(F)c2)nc1)C1CCCCC1)C(=O)N[C@@H](CCCNC(=N)N)C(=O)N[C@@H](CCCCN)C(N)=O]

MS: Calc'd for C₄₇H₇₃FN₁₂O₆: 920.58, found [M+H]⁺: 921.6.

### Example 113 (Compound 113) (SEQ ID NO: 273)

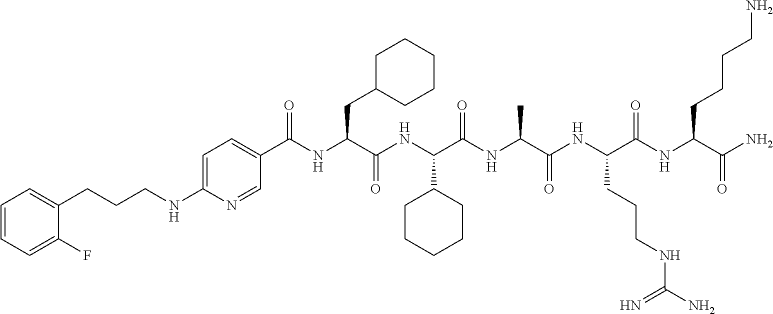 [SMILES: C[C@H](NC(=O)[C@@H](NC(=O)[C@H](CC1CCCCC1)NC(=O)c1ccc(NCCCc2ccccc2F)nc1)C1CCCCC1)C(=O)N[C@@H](CCCNC(=N)N)C(=O)N[C@@H](CCCCN)C(N)=O]

MS: Calc'd for C₄₇H₇₃FN₁₂O₆: 920.58, found [M+H]⁺: 921.6.

### Example 114 (Compound 114) (SEQ ID NO: 274)

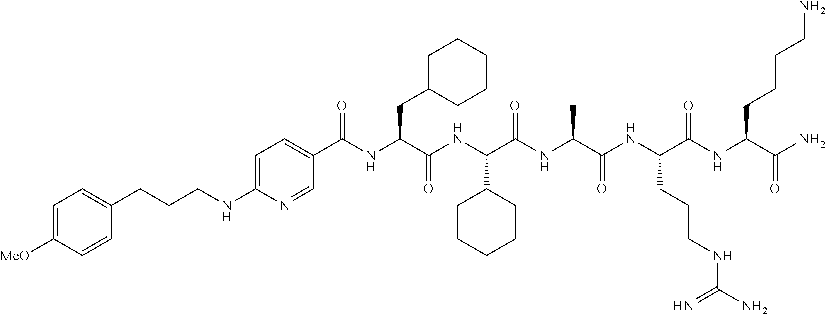 [SMILES: COc1ccc(CCCNc2ccc(C(=O)N[C@@H](CC3CCCCC3)C(=O)N[C@H](C(=O)N[C@@H](C)C(=O)N[C@@H](CCCNC(=N)N)C(=O)N[C@@H](CCCCN)C(N)=O)C3CCCCC3)cn2)cc1]

MS: Calc'd for C₄₈H₇₆N₁₂O₇: 932.60, found [M+H]⁺: 933.5.

### Example 115 (Compound 115) (SEQ ID NO: 275)

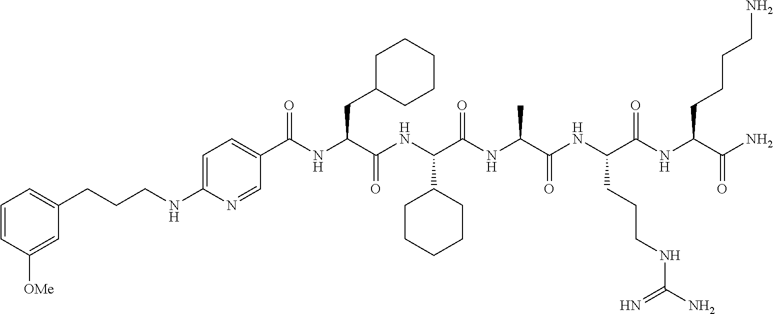 [SMILES: COc1cccc(CCCNc2ccc(C(=O)N[C@@H](CC3CCCCC3)C(=O)N[C@H](C(=O)N[C@@H](C)C(=O)N[C@@H](CCCNC(=N)N)C(=O)N[C@@H](CCCCN)C(N)=O)C3CCCCC3)cn2)c1]

MS: Calc'd for C₄₈H₇₆N₁₂O₇: 932.60, found [M+H]⁺: 933.6.

### Example 116 (Compound 116) (SEQ ID NO: 276)

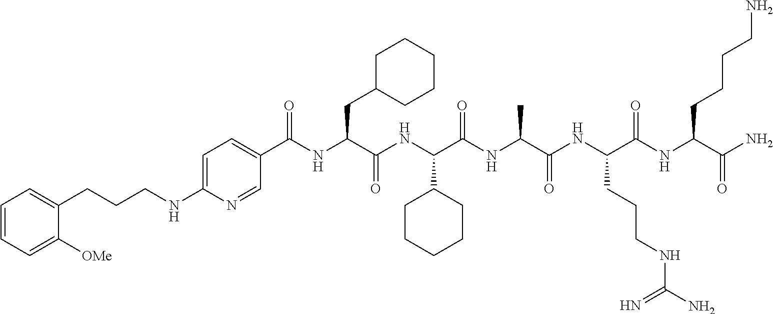 [SMILES: COc1ccccc1CCCNc1ccc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)cn1]

MS: Calc'd for C₄₈H₇₆N₁₂O₇: 932.60, found [M+H]⁺: 933.5.

### Example 117 (Compound 117) (SEQ ID NO: 277)

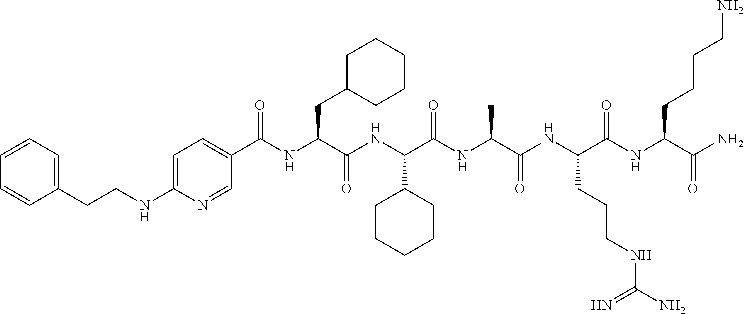 [SMILES: C[C@H](NC(=O)[C@@H](NC(=O)[C@H](CC1CCCCC1)NC(=O)c1ccc(NCCc2ccccc2)nc1)C1CCCCC1)C(=O)N[C@@H](CCCNC(=N)N)C(=O)N[C@@H](CCCCN)C(N)=O]

MS: Calc'd for C₄₆H₇₂N₁₂O₆: 888.57, found [M+H]⁺: 889.5.

### Example 118 (Compound 118) (SEQ ID NO: 278)

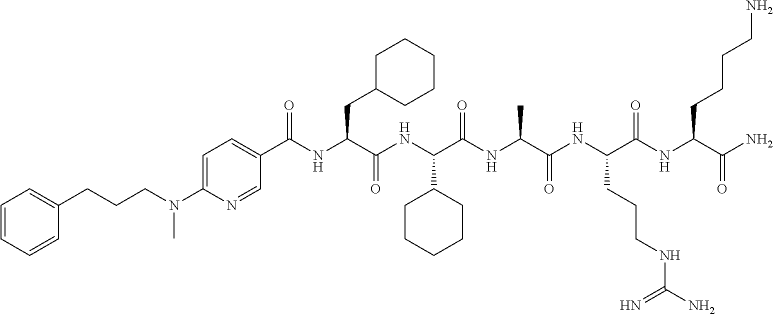 [SMILES: C[C@H](NC(=O)[C@@H](NC(=O)[C@H](CC1CCCCC1)NC(=O)c1ccc(N(C)CCCc2ccccc2)nc1)C1CCCCC1)C(=O)N[C@@H](CCCNC(=N)N)C(=O)N[C@@H](CCCCN)C(N)=O]

MS: Calc'd for C₄₈H₇₆N₁₂O₆: 916.60, found [M+H]⁺: 917.5.

### Example 119 (Compound 119) (SEQ ID NO: 279)

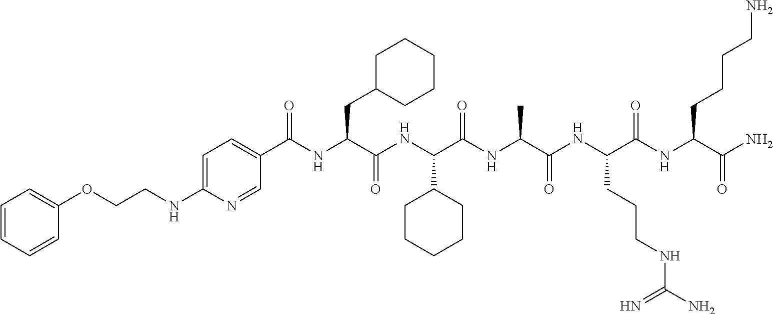 [SMILES: C[C@H](NC(=O)[C@@H](NC(=O)[C@H](CC1CCCCC1)NC(=O)c1ccc(NCCOc2ccccc2)nc1)C1CCCCC1)C(=O)N[C@@H](CCCNC(=N)N)C(=O)N[C@@H](CCCCN)C(N)=O]

MS: Calc'd for C₄₆H₂₂N₁₂O₇: 904.56, found [M+H]⁺: 905.5.

### Example 120 (Compound 120) (SEQ ID NO: 280)

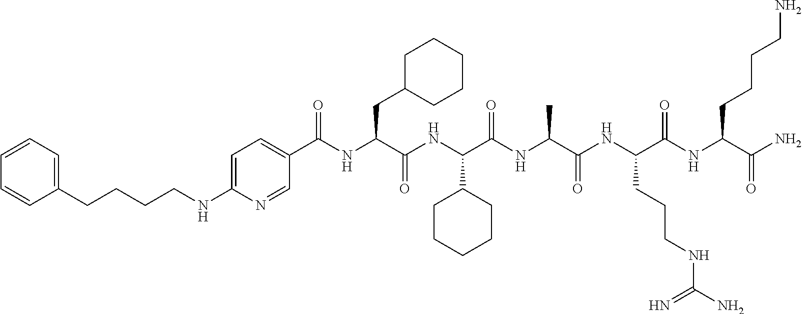 [SMILES: C[C@H](NC(=O)[C@@H](NC(=O)[C@H](CC1CCCCC1)NC(=O)c1ccc(NCCCCc2ccccc2)nc1)C1CCCCC1)C(=O)N[C@@H](CCCNC(=N)N)C(=O)N[C@@H](CCCCN)C(N)=O]

MS: Calc'd for C₄₈H₇₆N₁₂O₆: 916.60, found [M+H]⁺: 917.5.

### Example 121 (Compound 121) (SEQ ID NO: 281)

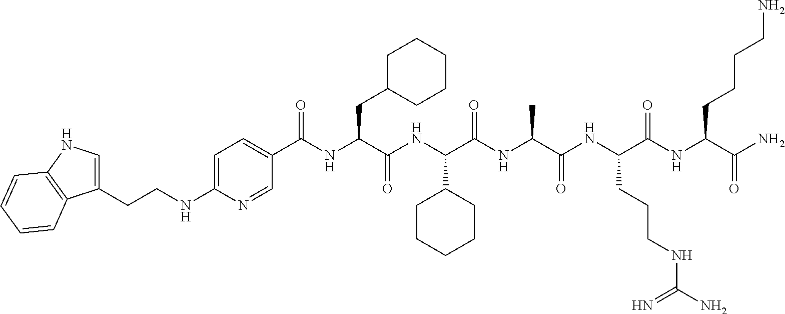 [SMILES: C[C@H](NC(=O)[C@@H](NC(=O)[C@H](CC1CCCCC1)NC(=O)c1ccc(NCCc2c[nH]c3ccccc23)nc1)C1CCCCC1)C(=O)N[C@@H](CCCNC(=N)N)C(=O)N[C@@H](CCCCN)C(N)=O]

MS: Calc'd for C₄₈H₇₃N₁₃O₆: 927.58, found [M+H]⁺: 928.5.

### Example 122 (Compound 122) (SEQ ID NO: 282)

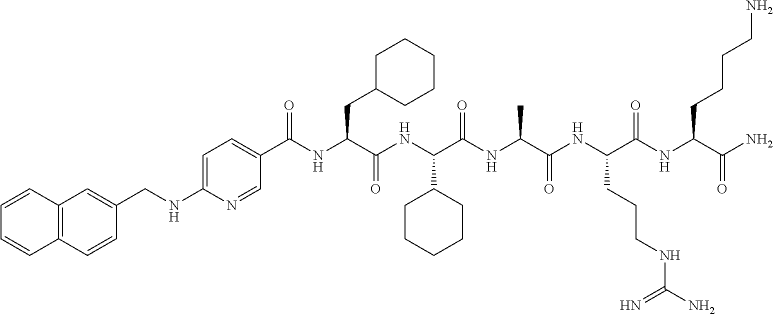 [SMILES: C[C@H](NC(=O)[C@@H](NC(=O)[C@H](CC1CCCCC1)NC(=O)c1ccc(NCc2ccc3ccccc3c2)nc1)C1CCCCC1)C(=O)N[C@@H](CCCNC(=N)N)C(=O)N[C@@H](CCCCN)C(N)=O]

MS: Calc'd for C₄₉H₇₂N₁₂O₆: 924.57, found [M+H]⁺: 925.5.

### Example 123 (Compound 123) (SEQ ID NO: 283)

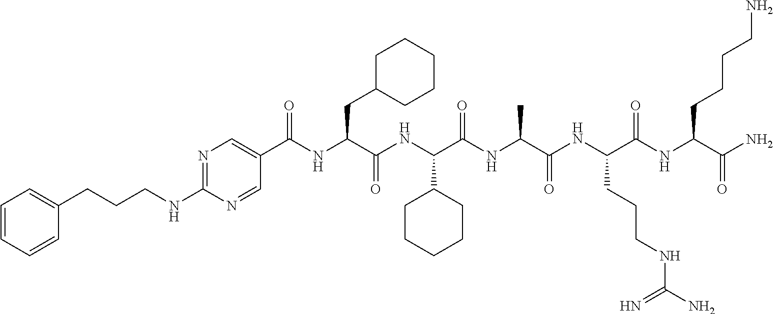 [SMILES: C[C@H](NC(=O)[C@@H](NC(=O)[C@H](CC1CCCCC1)NC(=O)c1cnc(NCCCc2ccccc2)nc1)C1CCCCC1)C(=O)N[C@@H](CCCNC(=N)N)C(=O)N[C@@H](CCCCN)C(N)=O]

MS: Calc'd for C₄₆H₇₃N₁₃O₆: 903.58, found [M+H]⁺: 904.6.

### Example 124 (Compound 124) (SEQ ID NO: 284)

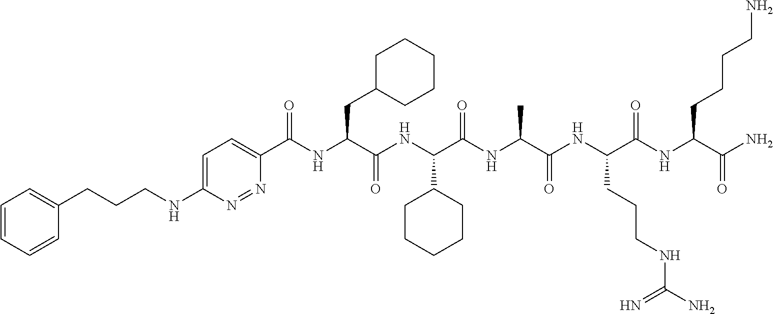 [SMILES: C[C@H](NC(=O)[C@@H](NC(=O)[C@H](CC1CCCCC1)NC(=O)c1ccc(NCCCc2ccccc2)nn1)C1CCCCC1)C(=O)N[C@@H](CCCNC(=N)N)C(=O)N[C@@H](CCCCN)C(N)=O]

MS: Calc'd for C₄₆H₇₃N₁₃O₆: 903.58, found [M+H]⁺: 904.6.

### Example 125 (Compound 125) (SEQ ID NO: 285)

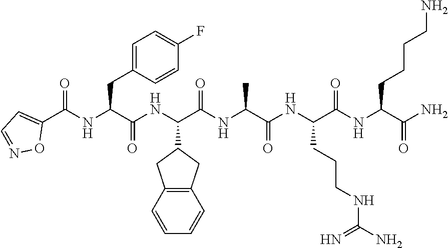 [SMILES: C[C@H](NC(=O)[C@@H](NC(=O)[C@H](Cc1ccc(F)cc1)NC(=O)c1ccno1)C1Cc2ccccc2C1)C(=O)N[C@@H](CCCNC(=N)N)C(=O)N[C@@H](CCCCN)C(N)=O]

MS: Calc'd for C₃₉H₅₂FN₁₁O₇: 805.40, found [M+H]⁺: 806.3.

### Example 126 (Compound 126) (SEQ ID NO: 286)

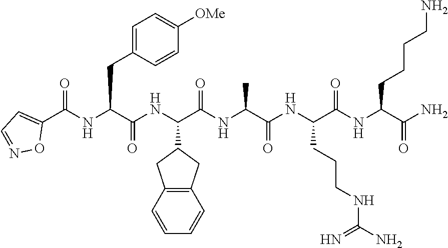 [SMILES: COc1ccc(C[C@H](NC(=O)c2ccno2)C(=O)N[C@H](C(=O)N[C@@H](C)C(=O)N[C@@H](CCCNC(=N)N)C(=O)N[C@@H](CCCCN)C(N)=O)C2Cc3ccccc3C2)cc1]

MS: Calc'd for C₄₀H₅₅N₁₁O₈: 817.42, found [M+2H]²⁺: 409.9.

### Example 127 (Compound 127) (SEQ ID NO: 287)

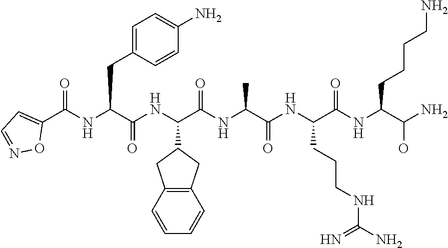 [SMILES: C[C@H](NC(=O)[C@@H](NC(=O)[C@H](Cc1ccc(N)cc1)NC(=O)c1ccno1)C1Cc2ccccc2C1)C(=O)N[C@@H](CCCNC(=N)N)C(=O)N[C@@H](CCCCN)C(N)O]

MS: Calc'd for C₃₉H₅₄N₁₂O₇: 802.42, found [M+2H]²⁺: 402.4.

### Example 128 (Compound 128) (SEQ ID NO: 288)

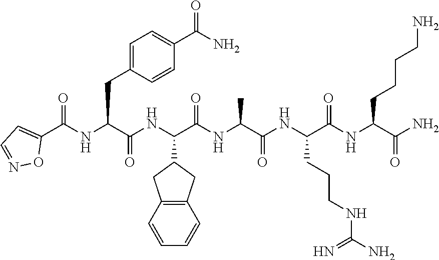 [SMILES: C[C@H](NC(=O)[C@@H](NC(=O)[C@H](Cc1ccc(C(N)=O)cc1)NC(=O)c1ccno1)C1Cc2ccccc2C1)C(=O)N[C@@H](CCCNC(=N)N)C(=O)N[C@@H](CCCCN)C(N)=O]

MS: Calc'd for C₄₀H₅₄N₁₂O₈: 830.42, found [M+2H]²⁺: 416.3.

### Example 129 (Compound 129) (SEQ ID NO: 289)

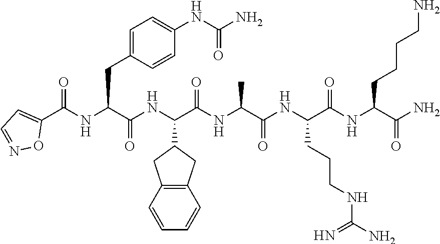 [SMILES: C[C@H](NC(=O)[C@@H](NC(=O)[C@H](Cc1ccc(NC(N)=O)cc1)NC(=O)c1ccno1)C1Cc2ccccc2C1)C(=O)N[C@@H](CCCNC(=N)N)C(=O)N[C@@H](CCCCN)C(N)=O]

MS: Calc'd for C₄₀H₅₅N₁₃O₈. 845.43, found [M+2H]²⁺: 423.8.

### Example 130 (Compound 130) (SEQ ID NO: 290)

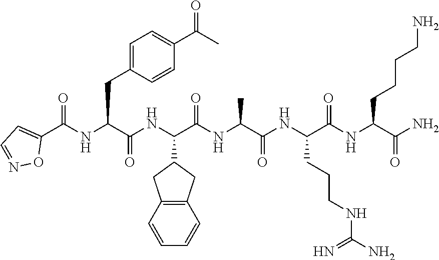 [SMILES: CC(=O)c1ccc(C[C@H](NC(=O)c2ccno2)C(=O)N[C@H](C(=O)N[C@@H](C)C(=O)N[C@@H](CCCNC(=N)N)C(=O)N[C@@H](CCCCN)C(N)=O)C2Cc3ccccc3C2)cc1]

MS: Calc'd for C₄₁H₅₅N₁₁O₈: 829.42, found [M+2H]²⁺: 415.9.

### Example 131 (Compound 131) (SEQ ID NO: 291)

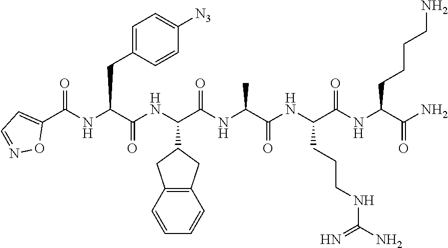 [SMILES: C[C@H](NC(=O)[C@@H](NC(=O)[C@H](Cc1ccc(N=[N+]=[N-])cc1)NC(=O)c1ccno1)C1Cc2ccccc2C1)C(=O)N[C@@H](CCCNC(=N)N)C(=O)N[C@@H](CCCCN)C(N)=O]

### Example 132 (Compound 132) (SEQ ID NO: 292)

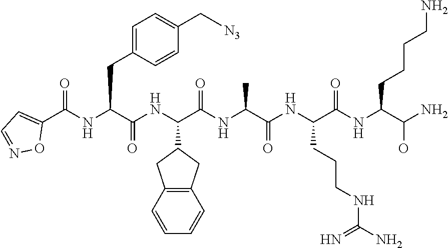 [SMILES: C[C@H](NC(=O)[C@@H](NC(=O)[C@H](Cc1ccc(CN=[N+]=[N-])cc1)NC(=O)c1ccno1)C1Cc2ccccc2C1)C(=O)N[C@@H](CCCNC(=N)N)C(=O)N[C@@H](CCCCN)C(N)O]

### Example 133 (Compound 133) (SEQ ID NO: 293)

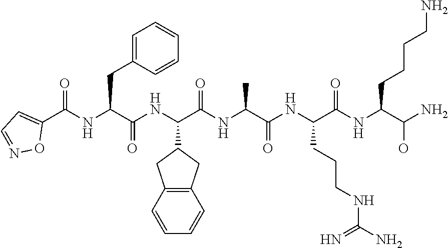 [SMILES: C[C@H](NC(=O)[C@@H](NC(=O)[C@H](Cc1ccccc1)NC(=O)c1ccno1)C1Cc2ccccc2C1)C(=O)N[C@@H](CCCNC(=N)N)C(=O)N[C@@H](CCCCN)C(N)O]

MS: Calc'd for C₃₉H₅₃N₁₁O₇: 787.41, found [M+2H]²⁺: 394.8.

### Example 134 (Compound 134) (SEQ ID NO: 294)

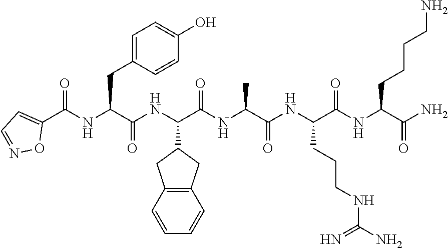 [SMILES: C[C@H](NC(=O)[C@@H](NC(=O)[C@H](Cc1ccc(O)cc1)NC(=O)c1ccno1)C1Cc2ccccc2C1)C(=O)N[C@@H](CCCNC(=N)N)C(=O)N[C@@H](CCCCN)C(N)=O]

MS: Calc'd for C₃₉H₅₅N₁₁O₈: 803.41, found [M+2H]²⁺: 402.9.

### Example 135 (Compound 135) (SEQ ID NO: 295)

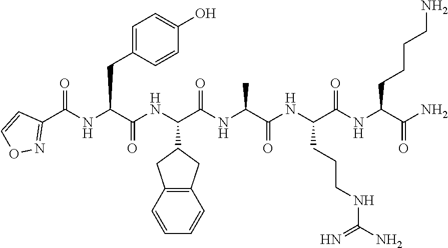 [SMILES: C[C@H](NC(=O)[C@@H](NC(=O)[C@H](Cc1ccc(O)cc1)NC(=O)c1ccon1)C1Cc2ccccc2C1)C(=O)N[C@@H](CCCNC(=N)N)C(=O)N[C@@H](CCCCN)C(N)=O]

MS: Calc'd for C₃₉H₅₃N₁₁O₈: 803.41, found [M+H]⁺: 804.3.

### Example 136 (Compound 136) (SEQ ID NO: 296)

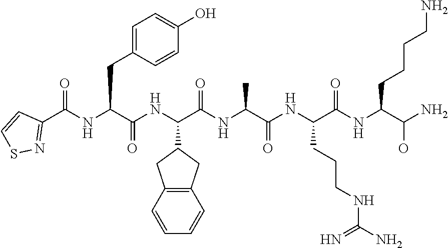 [SMILES: C[C@H](NC(=O)[C@@H](NC(=O)[C@H](Cc1ccc(O)cc1)NC(=O)c1ccsn1)C1Cc2ccccc2C1)C(=O)N[C@@H](CCCNC(=N)N)C(=O)N[C@@H](CCCCN)C(N)O]

MS: Calc'd for C₃₉H₅₃N₁₁O₇S: 819.39, found [M+H]⁺: 820.3.

### Example 137 (Compound 137) (SEQ ID NO: 297)

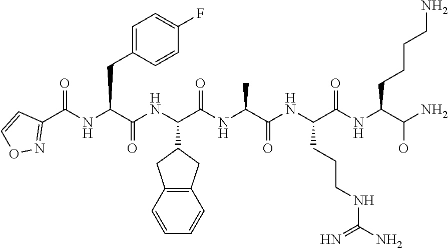 [SMILES: C[C@H](NC(=O)[C@@H](NC(=O)[C@H](Cc1ccc(F)cc1)NC(=O)c1ccon1)C1Cc2ccccc2C1)C(=O)N[C@@H](CCCNC(=N)N)C(=O)N[C@@H](CCCCN)C(N)O]

MS: Calc'd for C₃₉H₅₂FN₁₁O₇: 805.40, found [M+H]⁺: 806.4.

### Example 138 (Compound 138) (SEQ ID NO: 298)

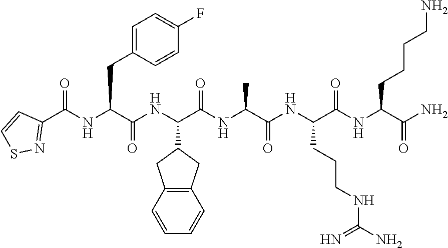 [SMILES: C[C@H](NC(=O)[C@@H](NC(=O)[C@H](Cc1ccc(F)cc1)NC(=O)c1ccsn1)C1Cc2ccccc2C1)C(=O)N[C@@H](CCCNC(=N)N)C(=O)N[C@@H](CCCCN)C(N)=O]

MS: Calc'd for C₃₉H₅₂FN₁₁O₆S: 821.38, found [M+H]⁺: 822.3.

### Example 139 (Compound 139) (SEQ ID NO: 299)

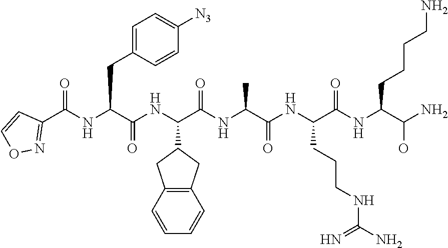 [SMILES: C[C@H](NC(=O)[C@@H](NC(=O)[C@H](Cc1ccc(N=[N+]=[N-])cc1)NC(=O)c1ccon1)C1Cc2ccccc2C1)C(=O)N[C@@H](CCCNC(=N)N)C(=O)N[C@@H](CCCCN)C(N)O]

### Example 140 (Compound 140) (SEQ ID NO: 300)

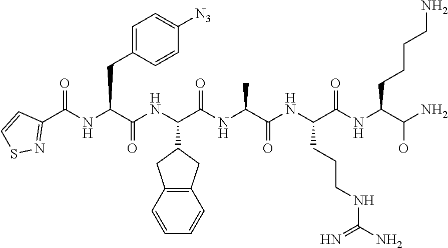 [SMILES: C[C@H](NC(=O)[C@@H](NC(=O)[C@H](Cc1ccc(N=[N+]=[N-])cc1)NC(=O)c1ccsn1)C1Cc2ccccc2C1)C(=O)N[C@@H](CCCNC(=N)N)C(=O)N[C@@H](CCCCN)C(N)O]

### Example 141 (Compound 141) (SEQ ID NO: 301)

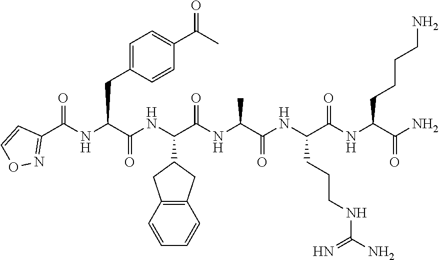 [SMILES: CC(=O)c1ccc(C[C@H](NC(=O)c2ccon2)C(=O)N[C@H](C(=O)N[C@@H](C)C(=O)N[C@@H](CCCNC(=N)N)C(=O)N[C@@H](CCCCN)C(N)=O)C2Cc3ccccc3C2)cc1]

MS: Calc'd for C₄₁H₅₅N₁₁O₈: 829.42, found [M+2H]²⁺: 415.9.

### Example 142 (Compound 142) (SEQ ID NO: 302)

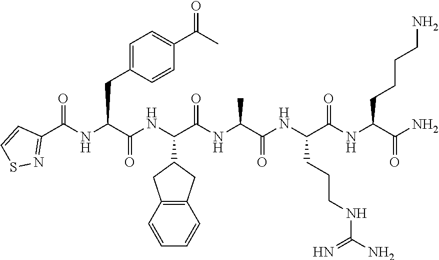 [SMILES: CC(=O)c1ccc(C[C@H](NC(=O)c2ccsn2)C(=O)N[C@H](C(=O)N[C@@H](C)C(=O)N[C@@H](CCCNC(=N)N)C(=O)N[C@@H](CCCCN)C(N)=O)C2Cc3ccccc3C2)cc1]

MS: Calc'd for C₄₁H₅₅N₁₁O₇S: 845.40, found [M+2H]²⁺: 423.8.

### Example 143 (Compound 143) (SEQ ID NO: 303)

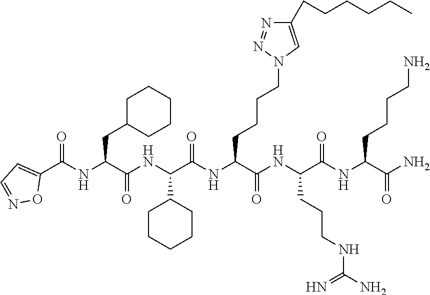 [SMILES: CCCCCCc1cn(CCCC[C@H](NC(=O)[C@@H](NC(=O)[C@H](CC2CCCCC2)NC(=O)c2ccno2)C2CCCCC2)C(=O)N[C@@H](CCCNC(=N)N)C(=O)N[C@@H](CCCCN)C(N)=O)nn1]

MS: Calc'd for C₄₇H₈₀N₁₄O₇: 952.63, found [M+2H]²⁺: 477.7.

### Example 144 (Compound 144) (SEQ ID NO: 304)

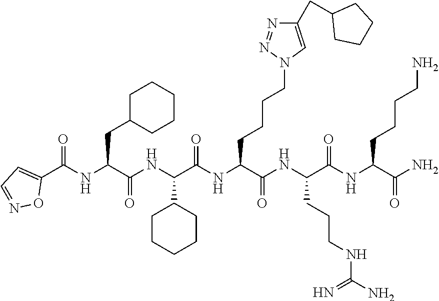 [SMILES: N=C(N)NCCC[C@H](NC(=O)[C@H](CCCCn1cc(CC2CCCC2)nn1)NC(=O)[C@@H](NC(=O)[C@H](CC1CCCCC1)NC(=O)c1ccno1)C1CCCCC1)C(=O)N[C@@H](CCCCN)C(N)=O]

MS: Calc'd for C₄₇H₇₈N₁₄O₇: 950.62, found [M+2H]²⁺: 476.7.

### Example 145 (Compound 145) (SEQ ID NO: 305)

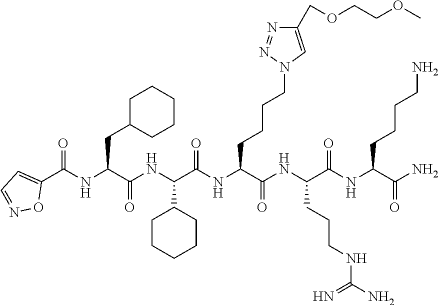 [SMILES: COCCOCc1cn(CCCC[C@H](NC(=O)[C@@H](NC(=O)[C@H](CC2CCCCC2)NC(=O)c2ccno2)C2CCCCC2)C(=O)N[C@@H](CCCNC(=N)N)C(=O)N[C@@H](CCCCN)C(N)=O)nn1]

MS: Calc'd for C₄₅H₇₆N₁₄O₈: 956.59, found [M+2H]²⁺: 479.6.

### Example 146 (Compound 146) (SEQ ID NO: 306)

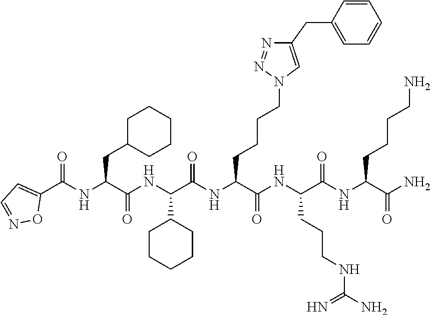 [SMILES: N=C(N)NCCC[C@H](NC(=O)[C@H](CCCCn1cc(Cc2ccccc2)nn1)NC(=O)[C@@H](NC(=O)[C@H](CC1CCCCC1)NC(=O)c1ccno1)C1CCCCC1)C(=O)N[C@@H](CCCCN)C(N)=O]

MS: Calc'd for C₄₈H₇₄N₁₄O₇: 958.59, found [M+2H]²⁺: 480.7.

### Example 147 (Compound 147) (SEQ ID NO: 307)

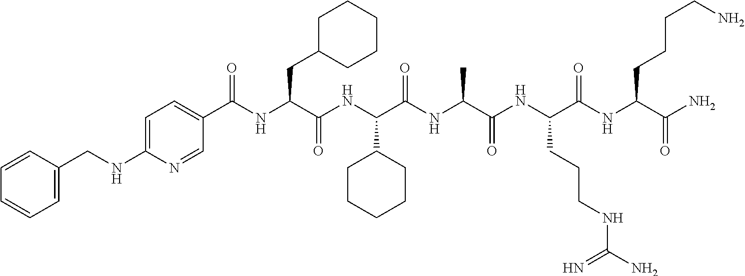 [SMILES: C[C@H](NC(=O)[C@@H](NC(=O)[C@H](CC1CCCCC1)NC(=O)c1ccc(NCc2ccccc2)nc1)C1CCCCC1)C(=O)N[C@@H](CCCNC(=N)N)C(=O)N[C@@H](CCCCN)C(N)=O]

MS: Calc'd for C₄₅H₇₀N₁₂O₆: 874.55, found [M+H]⁺: 875.6.

### Example 148 (Compound 148) (SEQ ID NO: 308)

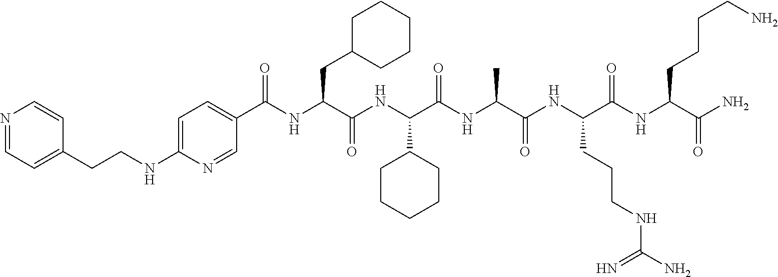 [SMILES: C[C@H](NC(=O)[C@@H](NC(=O)[C@H](CC1CCCCC1)NC(=O)c1ccc(NCCc2ccncc2)nc1)C1CCCCC1)C(=O)N[C@@H](CCCNC(=N)N)C(=O)N[C@@H](CCCCN)C(N)=O]

MS: Calc'd for C₄₅H₇₁N₁₃O₆: 889.56, found [M+H]⁺: 890.6.

### Example 149 (Compound 149) (SEQ ID NO: 309)

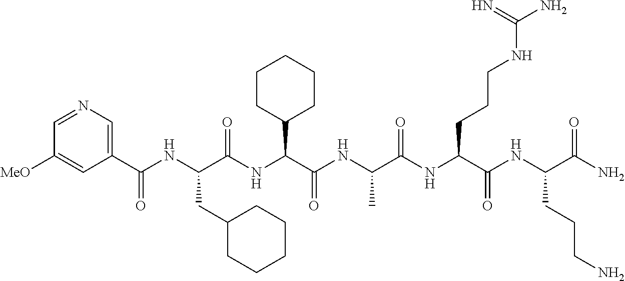 [SMILES: COc1cncc(C(=O)N[C@@H](CC2CCCCC2)C(=O)N[C@H](C(=O)N[C@@H](C)C(=O)N[C@@H](CCCNC(=N)N)C(=O)N[C@@H](CCCN)C(N)=O)C2CCCCC2)c1]

MS: Calc'd for C₃₈H₆₃N₁₁O₇: 785.49, found [M+2H]²⁺: 394.0.

### Example 150 (Compound 150) (SEQ ID NO: 310)

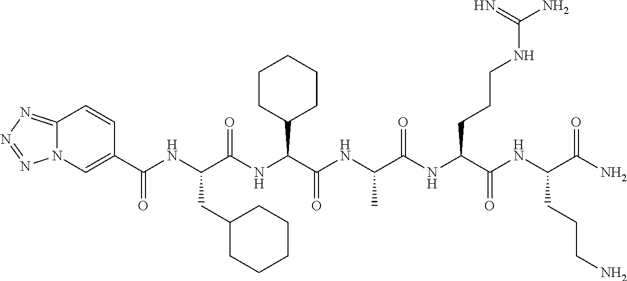 [SMILES: C[C@H](NC(=O)[C@@H](NC(=O)[C@H](CC1CCCCC1)NC(=O)c1ccc2nnnn2c1)C1CCCCC1)C(=O)N[C@@H](CCCNC(=N)N)C(=O)N[C@@H](CCCN)C(N)=O]

MS: Calc'd for C₃₇H₆₀N₁₄O₆: 796.48, found [M+2H]²⁺: 399.6.

### Example 151 (Compound 151) (SEQ ID NO: 311)

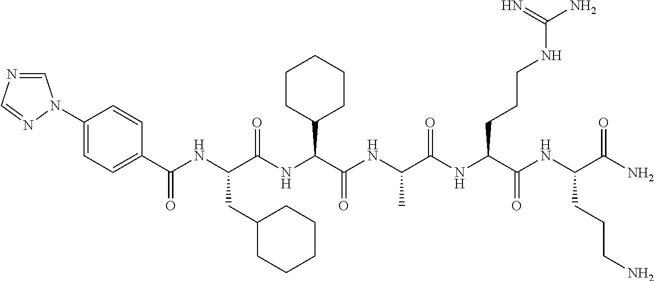 [SMILES: C[C@H](NC(=O)[C@@H](NC(=O)[C@H](CC1CCCCC1)NC(=O)c1ccc(-n2cncn2)cc1)C1CCCCC1)C(=O)N[C@@H](CCCNC(=N)N)C(=O)N[C@@H](CCCN)C(N)=O]

MS: Calc'd for C₄₀H₆₃N₁₃O₆: 821.50, found [M+2H]²⁺: 412.0.

### Example 152 (Compound 152) (SEQ ID NO: 312)

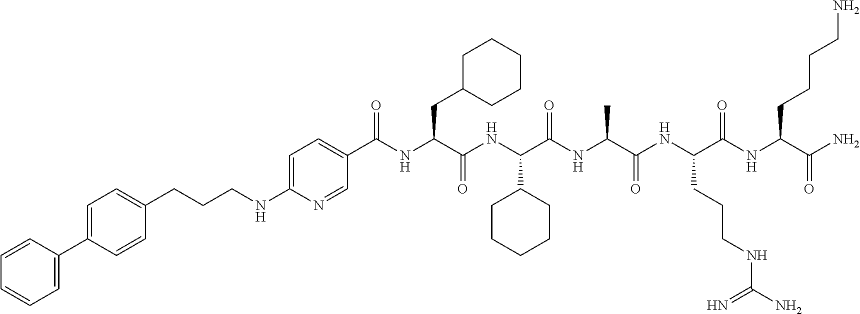 [SMILES: C[C@H](NC(=O)[C@@H](NC(=O)[C@H](CC1CCCCC1)NC(=O)c1ccc(NCCCc2ccc(-c3ccccc3)cc2)nc1)C1CCCCC1)C(=O)N[C@@H](CCCNC(=N)N)C(=O)N[C@@H](CCCCN)C(N)=O]

MS: Calc'd for C₅₃H₇₈N₁₂O₆: 978.62, found [M+H]⁺: 979.6.

### Example 153 (Compound 153) (SEQ ID NO: 313)

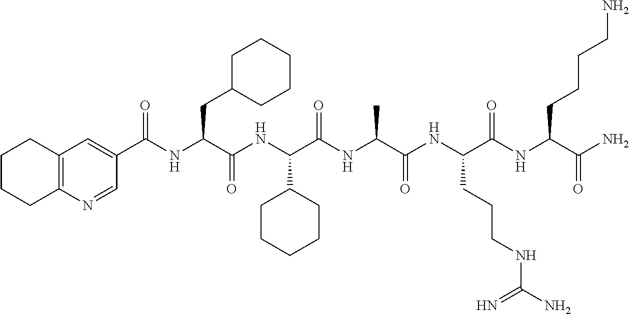 [SMILES: C[C@H](NC(=O)[C@@H](NC(=O)[C@H](CC1CCCCC1)NC(=O)c1cnc2c(c1)CCCC2)C1CCCCC1)C(=O)N[C@@H](CCCNC(=N)N)C(=O)N[C@@H](CCCCN)C(N)=O]

MS: Calc'd for C₄₂H₆₅N₁₁O₆: 819.51, found [M+H]⁺: 820.6.

### Example 154 (Compound 154) (SEQ ID NO: 314)

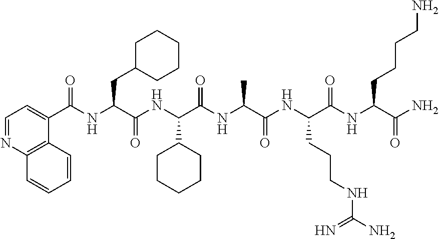 [SMILES: C[C@H](NC(=O)[C@@H](NC(=O)[C@H](CC1CCCCC1)NC(=O)c1ccnc2ccccc12)C1CCCCC1)C(=O)N[C@@H](CCCNC(=N)N)C(=O)N[C@@H](CCCCN)C(N)=O]

MS: Calc'd for C₄₂H₆₅N₁₁O₆: 819.51, found [M+H]⁺: 820.5.

### Example 155 (Compound 155) (SEQ ID NO: 315)

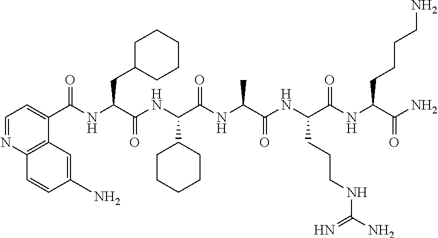 [SMILES: C[C@H](NC(=O)[C@@H](NC(=O)[C@H](CC1CCCCC1)NC(=O)c1ccnc2ccc(N)cc12)C1CCCCC1)C(=O)N[C@@H](CCCNC(=N)N)C(=O)N[C@@H](CCCCN)C(N)=O]

MS: Calc'd for C₄₂H₆₆N₁₂O₆: 834.52, found [M+H]⁺: 835.5.

### Example 156 (Compound 156) (SEQ ID NO: 316)

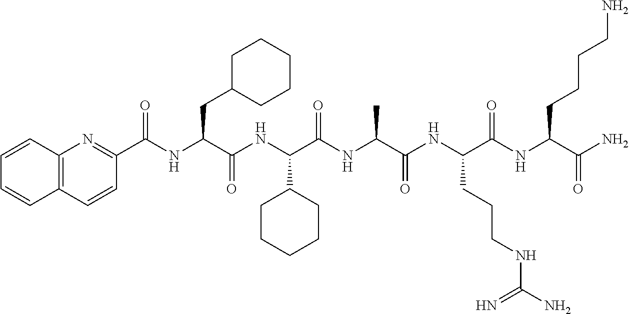 [SMILES: C[C@H](NC(=O)[C@@H](NC(=O)[C@H](CC1CCCCC1)NC(=O)c1ccc2ccccc2n1)C1CCCCC1)C(=O)N[C@@H](CCCNC(=N)N)C(=O)N[C@@H](CCCCN)C(N)=O]

MS: Calc'd for C₄₂H₆₅N₁₁O₆: 819.51, found [M+H]: 820.6.

### Example 157 (Compound 157) (SEQ ID NO: 317)

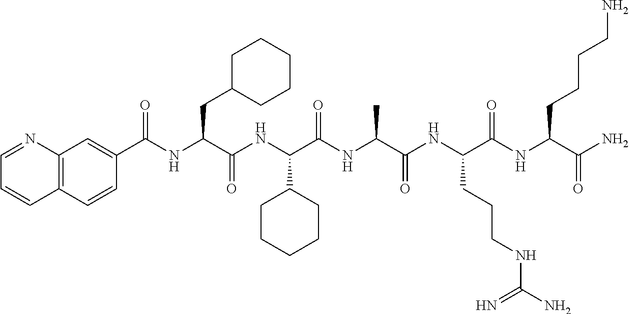 [SMILES: C[C@H](NC(=O)[C@@H](NC(=O)[C@H](CC1CCCCC1)NC(=O)c1ccc2cccnc2c1)C1CCCCC1)C(=O)N[C@@H](CCCNC(=N)N)C(=O)N[C@@H](CCCCN)C(N)=O]

MS: Calc'd for C₄₂H₆₅N₁₁O₆: 819.51, found [M+H]⁺: 820.6

### Example 158 (Compound 158) (SEQ ID NO: 318)

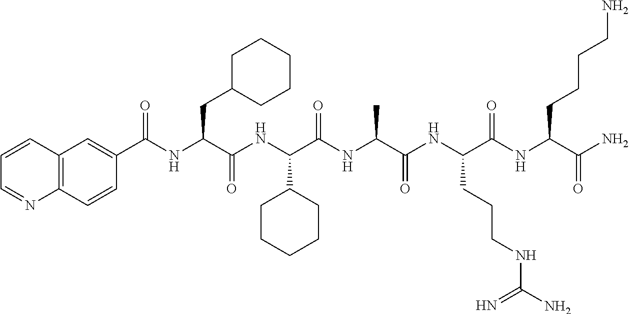 [SMILES: C[C@H](NC(=O)[C@@H](NC(=O)[C@H](CC1CCCCC1)NC(=O)c1ccc2ncccc2c1)C1CCCCC1)C(=O)N[C@@H](CCCNC(=N)N)C(=O)N[C@@H](CCCCN)C(N)=O]

MS: Calc'd for C₄₂H₆₅N₁₁O₆: 819.51, found [M+H]⁺: 820.6.

### Example 159 (Compound 159) (SEQ ID NO: 319)

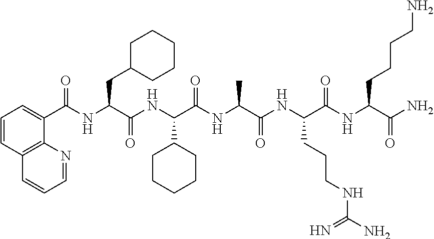 [SMILES: C[C@H](NC(=O)[C@@H](NC(=O)[C@H](CC1CCCCC1)NC(=O)c1cccc2cccnc12)C1CCCCC1)C(=O)N[C@@H](CCCNC(=N)N)C(=O)N[C@@H](CCCCN)C(N)=O]

MS: Calc'd for C₄₂H₆₅N₁₁O₆: 819.51, found [M+H]⁺: 820.6.

### Example 160 (Compound 160) (SEQ ID NO: 320)

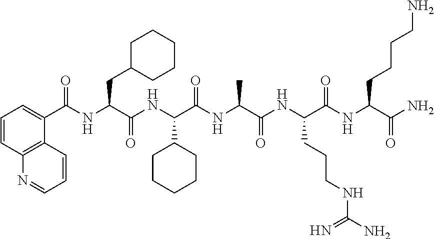 [SMILES: C[C@H](NC(=O)[C@@H](NC(=O)[C@H](CC1CCCCC1)NC(=O)c1cccc2ncccc12)C1CCCCC1)C(=O)N[C@@H](CCCNC(=N)N)C(=O)N[C@@H](CCCCN)C(N)=O]

MS: Calc'd for C₄₂H₆₅N₁₁O₆: 819.51, found [M+H]⁺: 820.6.

### Example 161 (Compound 161) (SEQ ID NO: 321)

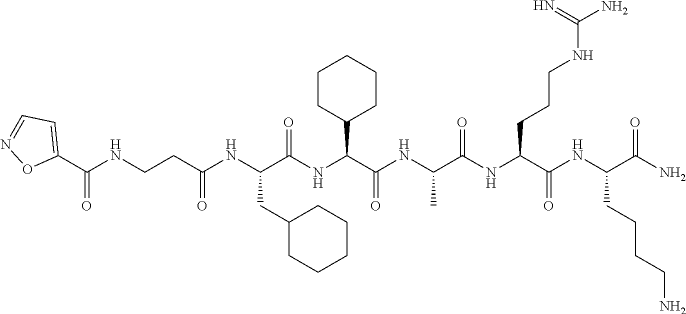 [SMILES: C[C@H](NC(=O)[C@@H](NC(=O)[C@H](CC1CCCCC1)NC(=O)CCNC(=O)c1ccno1)C1CCCCC1)C(=O)N[C@@H](CCCNC(=N)N)C(=O)N[C@@H](CCCCN)C(N)=O]

MS: Calc'd for C₃₉H₆₆N₁₂O₈: 830.51, found [M+H]⁺: 831.6.

### Example 162 (Compound 162) (SEQ ID NO: 322)

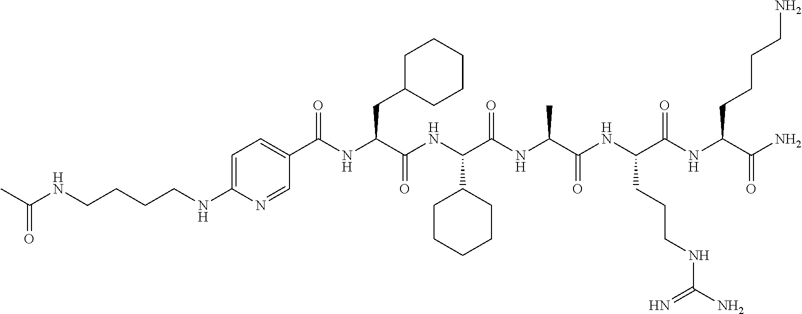 [SMILES: CC(=O)NCCCCNc1ccc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)cn1]

MS: Calc'd for C₄₄H₇₅N₁₃O₇: 897.59, found [M+H]⁺: 898.5.

### Example 163 (Compound 163)

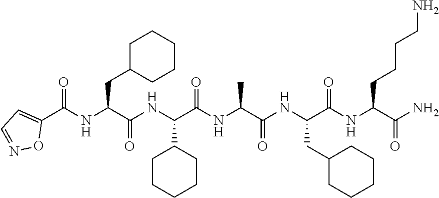 [SMILES: C[C@H](NC(=O)[C@@H](NC(=O)[C@H](CC1CCCCC1)NC(=O)c1ccno1)C1CCCCC1)C(=O)N[C@@H](CC1CCCCC1)C(=O)N[C@@H](CCCCN)C(N)=O]

### Example 164 (Compound 164) (SEQ ID NO: 324)

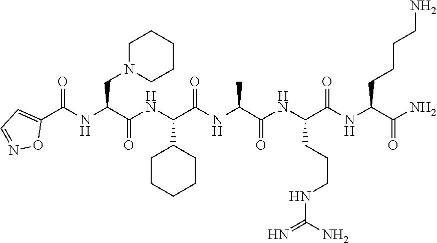 [SMILES: C[C@H](NC(=O)[C@@H](NC(=O)[C@H](CN1CCCCC1)NC(=O)c1ccno1)C1CCCCC1)C(=O)N[C@@H](CCCNC(=N)N)C(=O)N[C@@H](CCCCN)C(N)=O]

### Example 165 (Compound 165) (SEQ ID NO: 325)

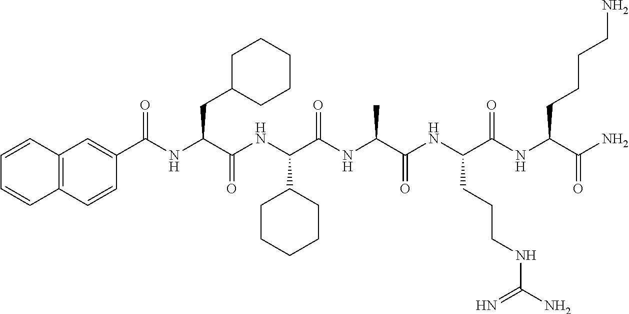 [SMILES: C[C@H](NC(=O)[C@@H](NC(=O)[C@H](CC1CCCCC1)NC(=O)c1ccc2ccccc2c1)C1CCCCC1)C(=O)N[C@@H](CCCNC(=N)N)C(=O)N[C@@H](CCCCN)C(N)=O]

MS: Calc'd for C₄₃H₆₆N₁₀O₆: 818.52, found [M+H]⁺: 819.4.

### Example 166 (Compound 166) (SEQ ID NO: 326)

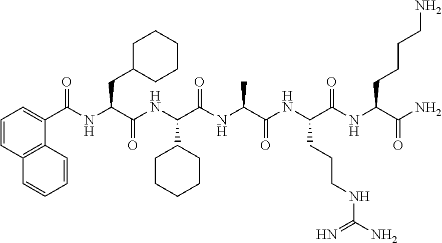 [SMILES: C[C@H](NC(=O)[C@@H](NC(=O)[C@H](CC1CCCCC1)NC(=O)c1cccc2ccccc12)C1CCCCC1)C(=O)N[C@@H](CCCNC(=N)N)C(=O)N[C@@H](CCCCN)C(N)=O]

MS: Calc'd for C₄₃H₆₆N₁₀O₆: 818.52, found [M+H]⁺: 819.4.

### Example 167 (Compound 167) (SEQ ID NO: 327)

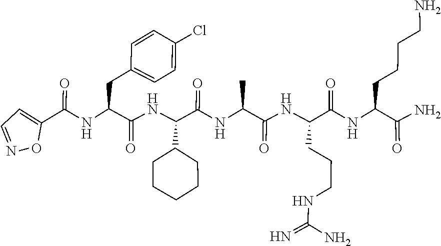

MS: Calc'd for C₃₆H₅₄ClN₁₁O₇: 787.39, found [M+H]⁺: 788.4.

### Example 168 (Compound 168) (SEQ ID NO: 328)

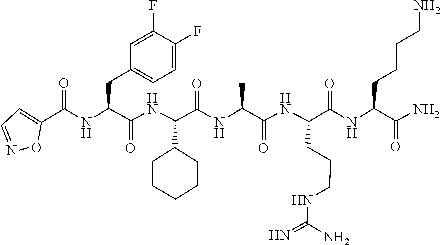

MS: Calc'd for C₃₆H₅₃F2N₁₁O₇: 789.41, found [M+H]⁺: 790.3.

### Example 169 (Compound 169) (SEQ ID NO: 329)

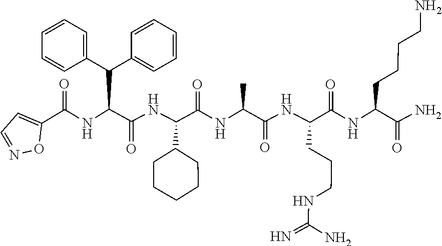 [SMILES: C[C@H](NC(=O)[C@@H](NC(=O)[C@@H](NC(=O)c1ccno1)C(c1ccccc1)c1ccccc1)C1CCCCC1)C(=O)N[C@@H](CCCNC(=N)N)C(=O)N[C@@H](CCCCN)C(N)=O]

MS: Calc'd for C₄₂H₅N₁₁O₇: 829.46, found [M+H]⁺: 830.3.

### Example 170 (Compound 170) (SEQ ID NO: 330)

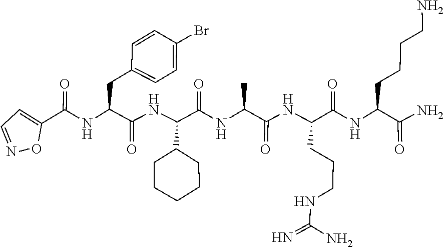

MS: Calc'd for C₃₆H₅₄BrN₁₁O₇: 831.34, found [M+2H]²⁺: 417.5.

### Example 171 (Compound 171) (SEQ ID NO: 331)

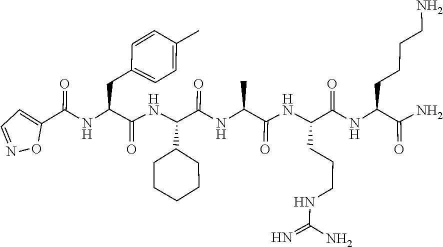

MS: Calc'd for C₃₇H₅₇N₁₁O₇: 767.44, found [M+2H]²⁺: 384.9.

### Example 172 (Compound 172) (SEQ ID NO: 332)

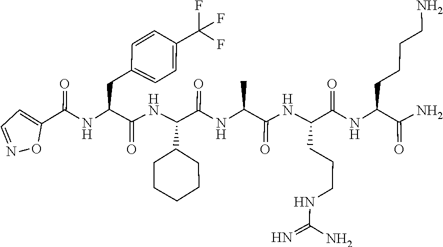

MS: Calc'd for C₇₅H₅₄F3N₁₁O₇: 821.42, found [M+2H]²⁺: 411.8.

### Example 173 (Compound 173) (SEQ ID NO: 333)

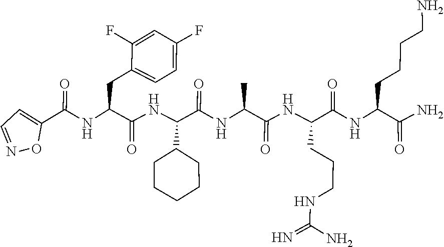

MS: Calc'd for C₃₆H₅₃F2N₁₁O₇: 789.41, found [M+2H]²⁺: 395.8.

### Example 174 (Compound 174) (SEQ ID NO: 334)

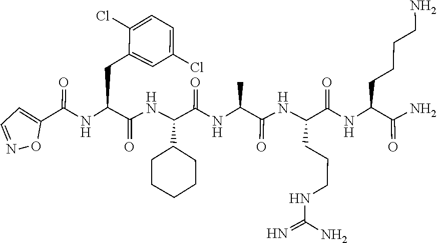

MS: Calc'd for C₃₆H₅₃C₁₂N₁₁O₇: 821.35, found [M+2H]²⁺: 412.3.

### Example 175 (Compound 175) (SEQ ID NO: 335)

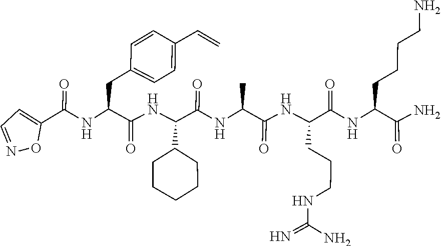

MS: Calc'd for C₃₈H₅₇N₁₁O₇: 779.44, found [M+2H]²⁺: 391.9.

### Example 176 (Compound 176) (SEQ ID NO: 336)

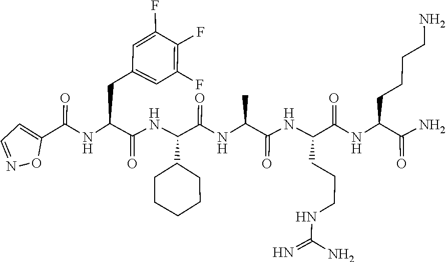

MS: Calc'd for C₃₆H₅₂F3N₁₁O₇: 807.40, found [M+2H]²⁺: 404.9.

### Example 177 (Compound 177) (SEQ ID NO: 337)

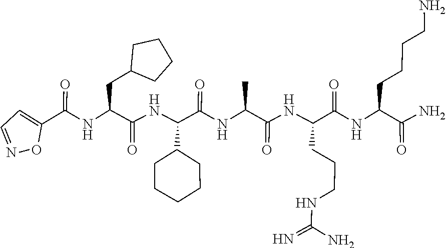

MS: Calc'd for C₃₅H₅N₁₁O₇: 745.46, found [M+2H]²⁺: 374.0.

### Example 178 (Compound 178) (SEQ ID NO: 338)

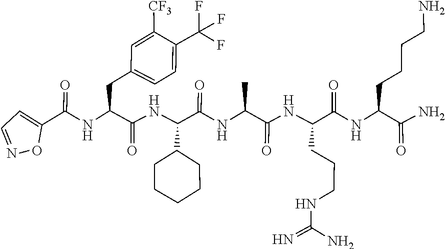

MS: Calc'd for C₃₈H₅₃F6N₁₁O₇: 889.40, found [M+H]⁺: 890.5.

### Example 179 (Compound 179) (SEQ ID NO: 339)

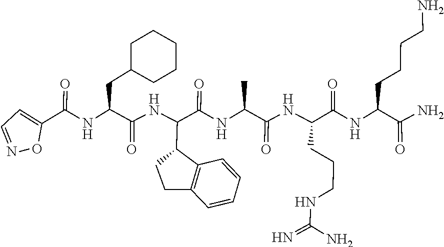

### Example 180 (Compound 180) (SEQ ID NO: 340)

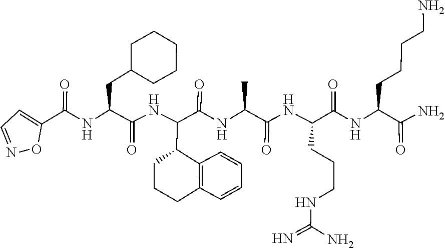

### Example 181 (Compound 181) (SEQ ID NO: 341)

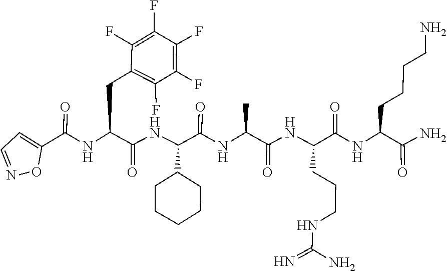

MS: Calc'd for C₃₆H₅₀F₅N₁₁O₇: 843.39, found [M+2H]²⁺: 422.9.

### Example 182 (Compound 182) (SEQ ID NO: 342)

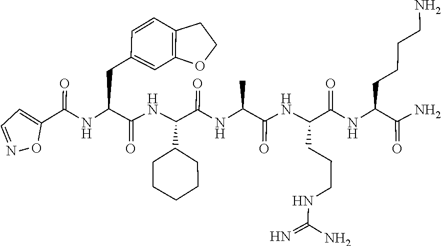

### Example 183 (Compound 183) (SEQ ID NO: 343)

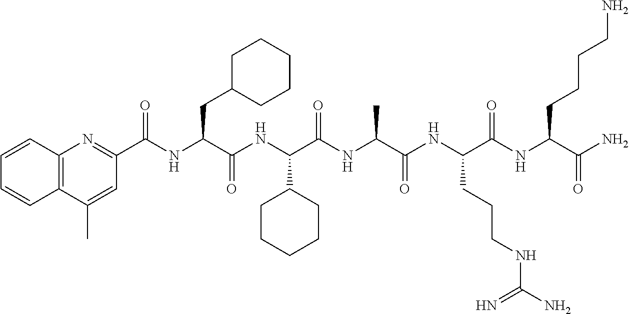 [SMILES: Cc1cc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)nc2ccccc12]

MS: Calc'd for C₄₃H₆₇N₁₁O₆: 833.53, found [M+2H]²⁺: 417.9.

### Example 184 (Compound 184) (SEQ ID NO: 344)

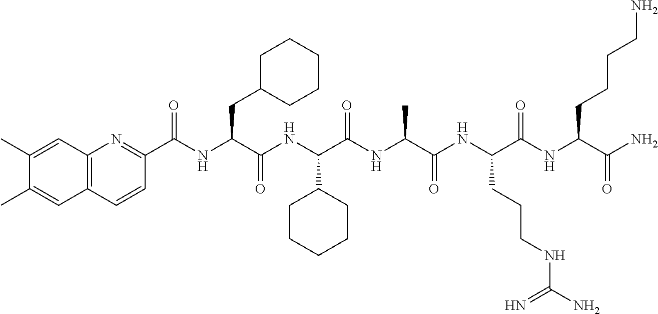 [SMILES: Cc1cc2ccc(C(=O)N[C@@H](CC3CCCCC3)C(=O)N[C@H](C(=O)N[C@@H](C)C(=O)N[C@@H](CCCNC(=N)N)C(=O)N[C@@H](CCCCN)C(N)=O)C3CCCCC3)nc2cc1C]

MS: Calc'd for C₄₃H₆₇N₁₁O₆: 833.53, found [M+2H]²⁺: 417.9.

### Example 185 (Compound 185) (SEQ ID NO: 345)

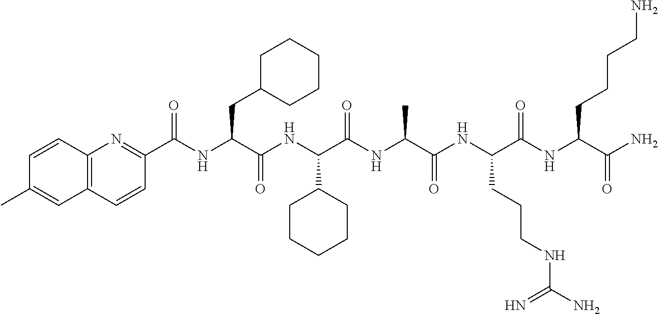 [SMILES: Cc1ccc2nc(C(=O)N[C@@H](CC3CCCCC3)C(=O)N[C@H](C(=O)N[C@@H](C)C(=O)N[C@@H](CCCNC(=N)N)C(=O)N[C@@H](CCCCN)C(N)=O)C3CCCCC3)ccc2c1]

MS: Calc'd for C₄₃H₆₇N₁₁O₆: 833.53, found [M+2H]²⁺: 417.9.

### Example 186 (Compound 186) (SEQ ID NO: 346)

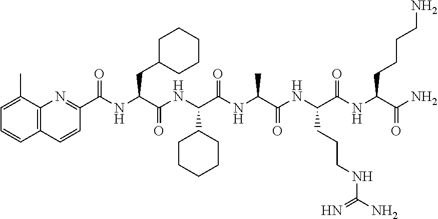 [SMILES: Cc1cccc2ccc(C(=O)N[C@@H](CC3CCCCC3)C(=O)N[C@H](C(=O)N[C@@H](C)C(=O)N[C@@H](CCCNC(=N)N)C(=O)N[C@@H](CCCCN)C(N)=O)C3CCCCC3)nc12]

MS: Calc'd for C₄₃H₆₇N₁₁O₆: 833.53, found [M+2H]²⁺: 418.1.

### Example 187 (Compound 187) (SEQ ID NO: 347)

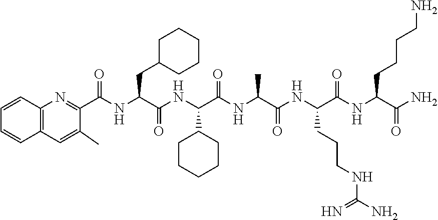 [SMILES: Cc1cc2ccccc2nc1C(=O)N[C@@H](CC1CCCCC1)C(=O)N[C@H](C(=O)N[C@@H](C)C(=O)N[C@@H](CCCNC(=N)N)C(=O)N[C@@H](CCCCN)C(N)=O)C1CCCCC1]

MS: Calc'd for C₄₃H₆₇N₁₁O₆: 833.53, found [M+2H]²⁺: 417.9.

### Example 188 (Compound 188) (SEQ ID NO: 348)

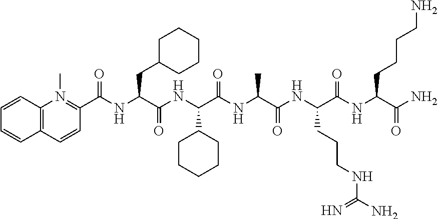

### Example 189 (Compound 189) (SEQ ID NO: 349)

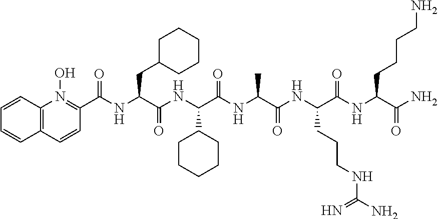

MS: Calc'd for C₄₂H₆₆N₁₁O₇: 836.51, found [M+2H]²⁺: 418.9.

### Example 190 (Compound 190) (SEQ ID NO: 350)

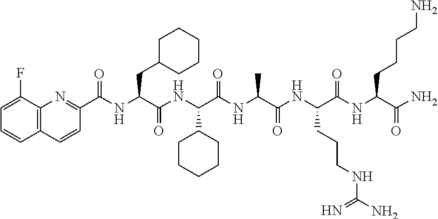 [SMILES: C[C@H](NC(=O)[C@@H](NC(=O)[C@H](CC1CCCCC1)NC(=O)c1ccc2cccc(F)c2n1)C1CCCCC1)C(=O)N[C@@H](CCCNC(=N)N)C(=O)N[C@@H](CCCCN)C(N)=O]

MS: Calc'd for C₄₂H₆₄FN₁₁O₆: 837.50, found [M+2H]²⁺: 419.8.

### Example 191 (Compound 191) (SEQ ID NO: 351)

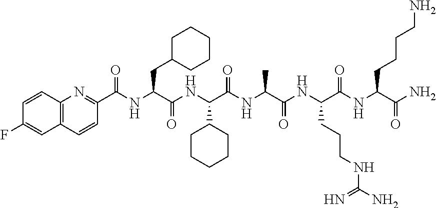 [SMILES: C[C@H](NC(=O)[C@@H](NC(=O)[C@H](CC1CCCCC1)NC(=O)c1ccc2cc(F)ccc2n1)C1CCCCC1)C(=O)N[C@@H](CCCNC(=N)N)C(=O)N[C@@H](CCCCN)C(N)=O]

MS: Calc'd for C₄₂H₆₄FN₁₁O₆: 837.50, found [M+2H]²⁺: 420.1.

### Example 192 (Compound 192) (SEQ ID NO: 352)

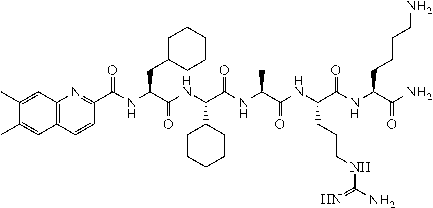 [SMILES: Cc1cc2ccc(C(=O)N[C@@H](CC3CCCCC3)C(=O)N[C@H](C(=O)N[C@@H](C)C(=O)N[C@@H](CCCNC(=N)N)C(=O)N[C@@H](CCCCN)C(N)=O)C3CCCCC3)nc2cc1C]

MS: Calc'd for C₄₄H₆₉N₁₁O₆: 847.54, found [M+2H]²⁺: 425.2.

### Example 193 (Compound 193) (SEQ ID NO: 353)

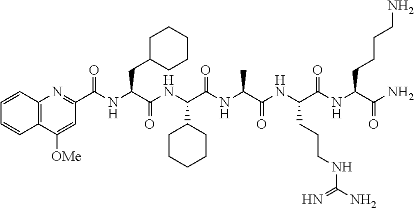 [SMILES: COc1cc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)nc2ccccc12]

MS: Calc'd for C₄₃H₆₇N₁₁O₇: 839.52, found [M+2H]²⁺: 426.0.

### Example 194 (Compound 194) (SEQ ID NO: 354)

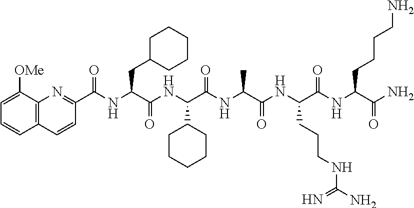 [SMILES: COc1cccc2ccc(C(=O)N[C@@H](CC3CCCCC3)C(=O)N[C@H](C(=O)N[C@@H](C)C(=O)N[C@@H](CCCNC(=N)N)C(=O)N[C@@H](CCCCN)C(N)=O)C3CCCCC3)nc12]

MS: Calc'd for C₄₃H₆₇N₁₁O₇: 849.52, found [M+2H]²⁺: 426.1.

### Example 195 (Compound 195) (SEQ ID NO: 355)

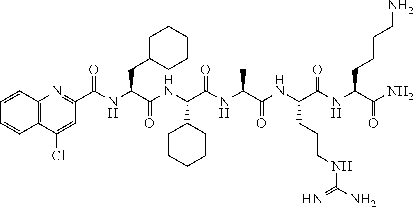 [SMILES: C[C@H](NC(=O)[C@@H](NC(=O)[C@H](CC1CCCCC1)NC(=O)c1cc(Cl)c2ccccc2n1)C1CCCCC1)C(=O)N[C@@H](CCCNC(=N)N)C(=O)N[C@@H](CCCCN)C(N)=O]

MS: Calc'd for C₄₂H₆₄ClN₁₁O₆: 853.47, found [M+2H]²⁺: 428.3.

### Example 196 (Compound 196) (SEQ ID NO: 356)

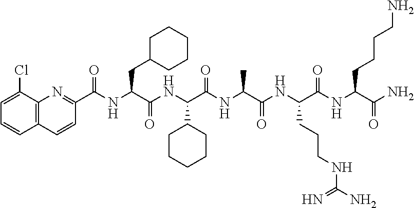 [SMILES: C[C@H](NC(=O)[C@@H](NC(=O)[C@H](CC1CCCCC1)NC(=O)c1ccc2cccc(Cl)c2n1)C1CCCCC1)C(=O)N[C@@H](CCCNC(=N)N)C(=O)N[C@@H](CCCCN)C(N)=O]

MS: Calc'd for C₄₂H₆₄ClN₁₁O₆: 853.47, found [M+2H]²⁺: 428.2.

### Example 197 (Compound 197) (SEQ ID NO: 357)

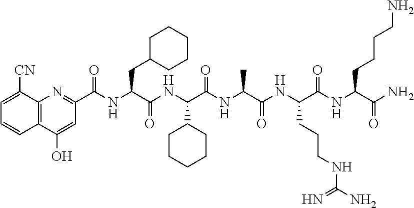 [SMILES: C[C@H](NC(=O)[C@@H](NC(=O)[C@H](CC1CCCCC1)NC(=O)c1cc(O)c2cccc(C#N)c2n1)C1CCCCC1)C(=O)N[C@@H](CCCNC(=N)N)C(=O)N[C@@H](CCCCN)C(N)=O]

### Example 198 (Compound 198) (SEQ ID NO: 358)

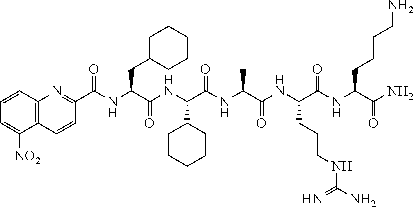 [SMILES: C[C@H](NC(=O)[C@@H](NC(=O)[C@H](CC1CCCCC1)NC(=O)c1ccc2c([N+](=O)[O-])cccc2n1)C1CCCCC1)C(=O)N[C@@H](CCCNC(=N)N)C(=O)N[C@@H](CCCCN)C(N)=O]

MS: Calc'd for C₄₂H₆₄N₁₂O₈: 864.50, found [M+2H]²⁺: 433.6.

### Example 199 (Compound 199) (SEQ ID NO: 359)

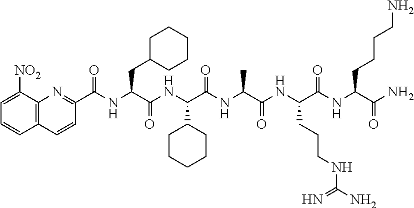 [SMILES: C[C@H](NC(=O)[C@@H](NC(=O)[C@H](CC1CCCCC1)NC(=O)c1ccc2cccc([N+](=O)[O-])c2n1)C1CCCCC1)C(=O)N[C@@H](CCCNC(=N)N)C(=O)N[C@@H](CCCCN)C(N)=O]

MS: Calc'd for C₄₂H₆₄N₁₂O₈: 864.50, found [M+2H]²⁺: 433.6.

### Example 200 (Compound 200) (SEQ ID NO: 360)

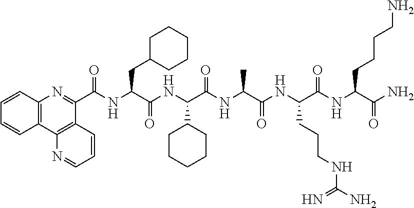 [SMILES: C[C@H](NC(=O)[C@@H](NC(=O)[C@H](CC1CCCCC1)NC(=O)c1nc2ccccc2c2ncccc12)C1CCCCC1)C(=O)N[C@@H](CCCNC(=N)N)C(=O)N[C@@H](CCCCN)C(N)=O]

MS: Calc'd for C₄₅H₆₆N₁₂O₆: 870.52, found [M+2H]²⁺: 436.6.

### Example 201 (Compound 201) (SEQ ID NO: 361)

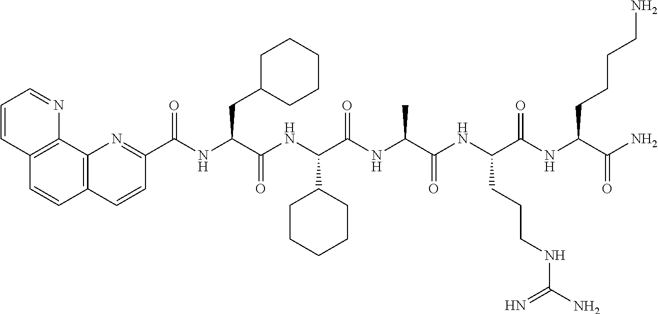 [SMILES: C[C@H](NC(=O)[C@@H](NC(=O)[C@H](CC1CCCCC1)NC(=O)c1ccc2ccc3cccnc3c2n1)C1CCCCC1)C(=O)N[C@@H](CCCNC(=N)N)C(=O)N[C@@H](CCCCN)C(N)=O]

MS: Calc'd for C₄₅H₆₆N₁₂O₆: 870.52, found [M+2H]²⁺: 436.4.

### Example 202 (Compound 202) (SEQ ID NO: 362)

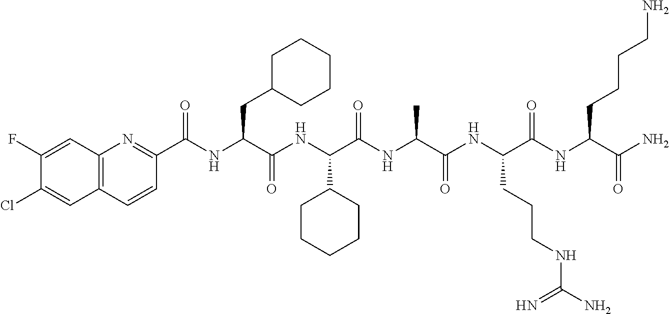 [SMILES: C[C@H](NC(=O)[C@@H](NC(=O)[C@H](CC1CCCCC1)NC(=O)c1ccc2cc(Cl)c(F)cc2n1)C1CCCCC1)C(=O)N[C@@H](CCCNC(=N)N)C(=O)N[C@@H](CCCCN)C(N)=O]

MS: Calc'd for C₄₂H₆₃ClN₁₁O₆: 871.46, found [M+2H]²⁺: 437.0.

### Example 203 (Compound 203) (SEQ ID NO: 363)

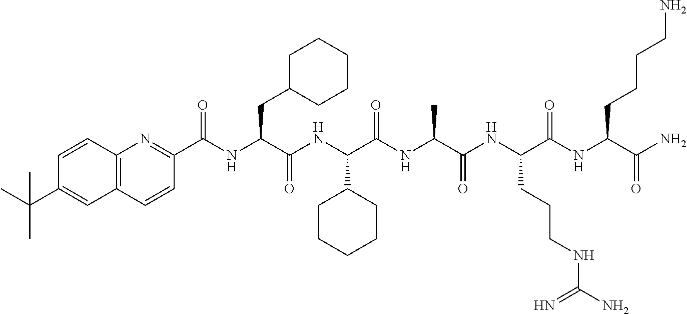 [SMILES: C[C@H](NC(=O)[C@@H](NC(=O)[C@H](CC1CCCCC1)NC(=O)c1ccc2cc(C(C)(C)C)ccc2n1)C1CCCCC1)C(=O)N[C@@H](CCCNC(=N)N)C(=O)N[C@@H](CCCCN)C(N)=O]

MS: Calc'd for C₄₆H₃N₁₁O₆: 875.57, found [M+2H]²⁺: 439.1.

### Example 204 (Compound 204) (SEQ ID NO: 364)

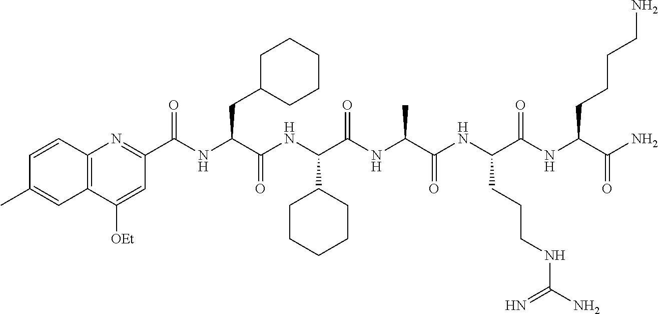 [SMILES: CCOc1cc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)nc2ccc(C)cc12]

MS: Calc'd for C₄₅H₇₁N₁₁O₇: 877.55, found [M+2H]²⁺: 440.1.

### Example 205 (Compound 205) (SEQ ID NO: 365)

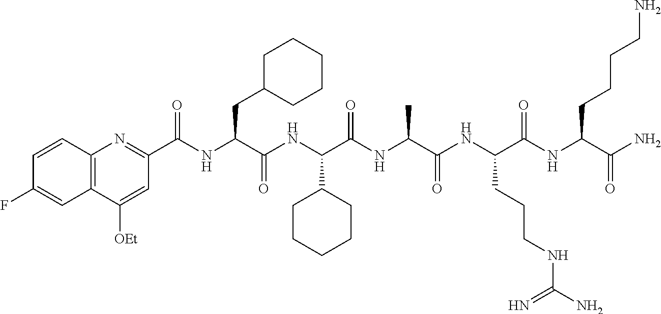 [SMILES: CCOc1cc(C(=O)N[C@@H](CC2CCCCC2)C(=O)N[C@H](C(=O)N[C@@H](C)C(=O)N[C@@H](CCCNC(=N)N)C(=O)N[C@@H](CCCCN)C(N)=O)C2CCCCC2)nc2ccc(F)cc12]

### Example 206 (Compound 206) (SEQ ID NO: 366)

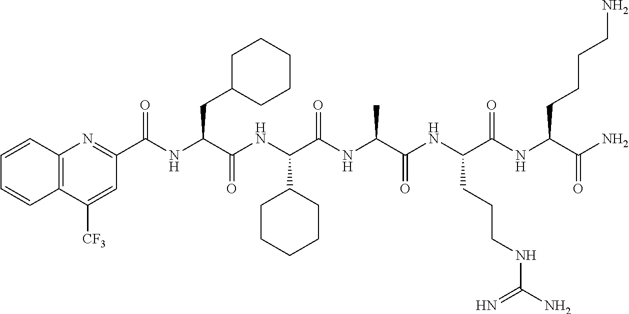 [SMILES: C[C@H](NC(=O)[C@@H](NC(=O)[C@H](CC1CCCCC1)NC(=O)c1cc(C(F)(F)F)c2ccccc2n1)C1CCCCC1)C(=O)N[C@@H](CCCNC(=N)N)C(=O)N[C@@H](CCCCN)C(N)=O]

MS: Calc'd for C₄₃H₆₄F3N₁₁O₆: 887.50, found [M+2H]²⁺: 444.9.

### Example 207 (Compound 207) (SEQ ID NO: 367)

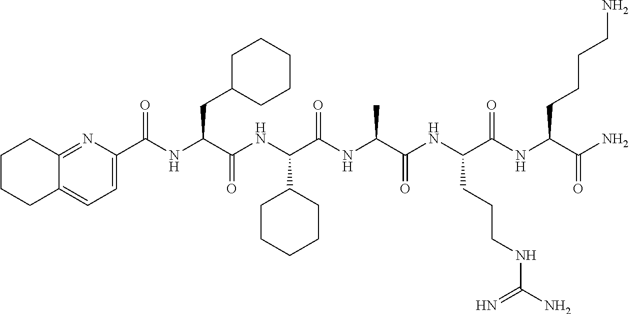 [SMILES: C[C@H](NC(=O)[C@@H](NC(=O)[C@H](CC1CCCCC1)NC(=O)c1ccc2c(n1)CCCC2)C1CCCCC1)C(=O)N[C@@H](CCCNC(=N)N)C(=O)N[C@@H](CCCCN)C(N)=O]

MS: Calc'd for C₄₂H₆₉N₁₁O₆: 823.54, found [M+2H]²⁺: 413.2.

### Example 208 (Compound 208) (SEQ ID NO: 368)

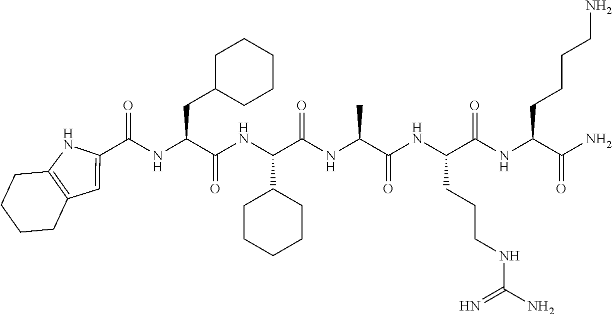 [SMILES: C[C@H](NC(=O)[C@@H](NC(=O)[C@H](CC1CCCCC1)NC(=O)c1cc2c([nH]1)CCCC2)C1CCCCC1)C(=O)N[C@@H](CCCNC(=N)N)C(=O)N[C@@H](CCCCN)C(N)=O]

MS: Calc'd for C₄₁H₆₉N₁₁O₆: 811.54, found [M+2H]²⁺: 407.1.

### Example 209 (Compound 209) (SEQ ID NO: 369)

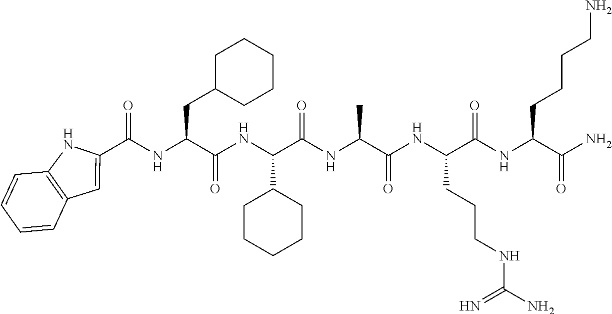 [SMILES: C[C@H](NC(=O)[C@@H](NC(=O)[C@H](CC1CCCCC1)NC(=O)c1cc2ccccc2[nH]1)C1CCCCC1)C(=O)N[C@@H](CCCNC(=N)N)C(=O)N[C@@H](CCCCN)C(N)=O]

MS: Calc'd for C₄₁H₆₅N₁₁O₆: 807.52, found [M+2H]²⁺: 405.1.

### Example 210 (Compound 210) (SEQ ID NO: 370)

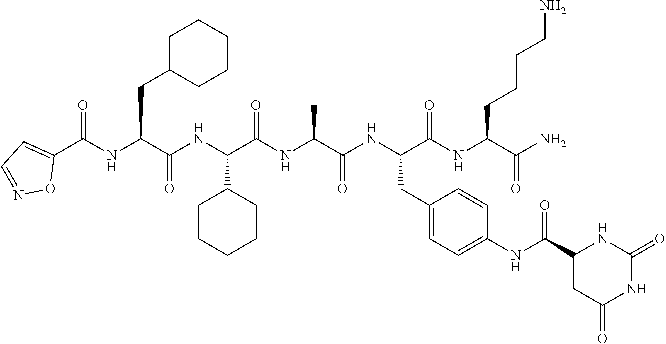

MS: Calc'd for C₄₄H₆₃N₁₁O₁₀: 905.48, found [M+H]⁺: 906.6.

### Example 219: Compound 219 (SEQ ID NO: 371)

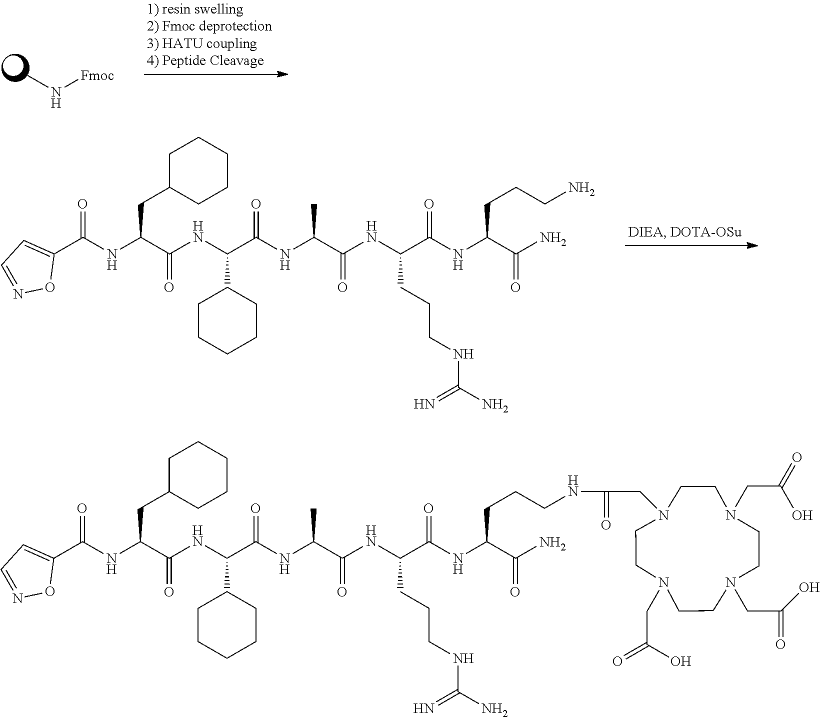

Steps 1-6: N-((2S,5S,8S,11S,14S)-17-amino-14-carbamoyl-1,5-dicyclohexyl-11-(3-guanidinopropyl)-8-methyl-3,6,9,12-tetraoxo-4,7,10,13-tetraazaheptadecan-2-yl) isoxazole-5-carboxamide (Compound 1) was prepared by standard Fmoc-based SPPS procedures from Sieber resin (General SPPS procedure 1-3). MS: Calc'd for C₃₅H₅N₁₁O₇: 745.46, found [M+H]⁺: 746.4.

Step 7: A mixture of N-((2S,5S,8S,11S,14S)-17-amino-14-carbamoyl-1,5-dicyclohexyl-11-(3-guanidinopropyl)-8-methyl-3,6,9,12-tetraoxo-4,7,10,13-tetraazaheptadecan-2-yl) isoxazole-5-carboxamide (Compound 1, 65 mg, 1 Eq, 87 μmol), 2,2′,2″-(10-(2-((2,5-dioxopyrrolidin-1-yl)oxy)-2-oxoethyl)-1,4,7,10-tetraazacyclododecane-1,4,7-triyl)triacetic acid (87 mg, 2.0 Eq, 0.17 mmol), DIEA (34 mg, 46 μL, 3.0 Eq, 0.26 mmol) and DMF (0.6 mL) was stirred at 25° C. for 1 hour. The crude product was purified by Prep-HPLC with the following conditions: Column: SunFire prep OBD 19*150 mm 5 μm; Mobile Phase A: Water (0.05% TFA); Mobile Phase B: ACN; Gradient: 25% B to 65% B in 8 min; Flow rate: 20 mL/min; Wave Length: 220 nm. The collected fractions were dried by lyophilization to provide the 2,2,2-trifluoroacetic acid salt of 2,2′,2″-(10-((3S,6S,9S,12S,15S)-15-carbamoyl-6-cyclohexyl-3-(cyclohexylmethyl)-12-(3-guanidinopropyl)-1-(isoxazol-5-yl)-9-methyl-1,4,7,10,13,20-hexaoxo-2,5,8,11,14,19-hexaazahenicosan-21-yl)-1,4,7,10-tetraazacyclododecane-1,4,7-triyl)triacetic acid (32.9 mg, 26.4 μmol, 30%) as a white solid. MS: Calc'd for C₅₁H₈₅N₁₅O₁₄: 1131.64, found [M+H]⁺: 1132.5.

### Example 220: Compound 220 (SEQ ID NO: 372)

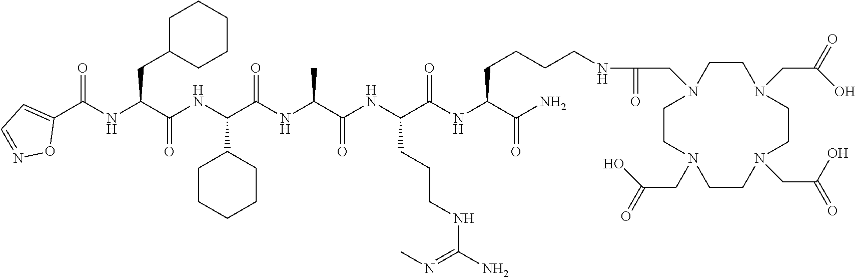 [SMILES: C/N=C(/N)NCCC[C@H](NC(=O)[C@H](C)NC(=O)[C@@H](NC(=O)[C@H](CC1CCCCC1)NC(=O)c1ccno1)C1CCCCC1)C(=O)N[C@@H](CCCCNC(=O)CN1CCN(CC(=O)O)CCN(CC(=O)O)CCN(CC(=O)O)CC1)C(N)=O]

MS: Calc'd for C₅₃H₈₉N₁₅O₁₄: 1159.67, found [M+3H]⁺: 387.85.

### Example 221: Compound 221 (SEQ ID NO: 373)

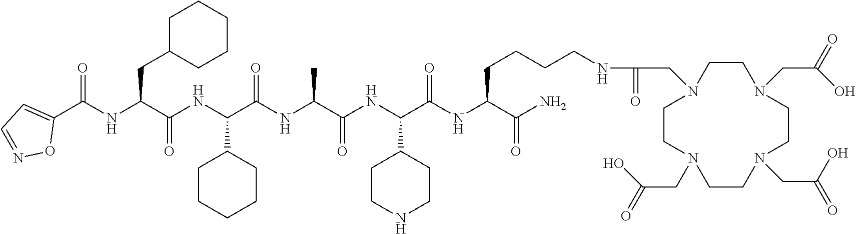 [SMILES: C[C@H](NC(=O)[C@@H](NC(=O)[C@H](CC1CCCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₅₃H₈₇N₁₃O₁₄: 1129.65, found [M+2H]²⁺: 566.2.

### Example 222: Compound 222 (SEQ ID NO: 374)

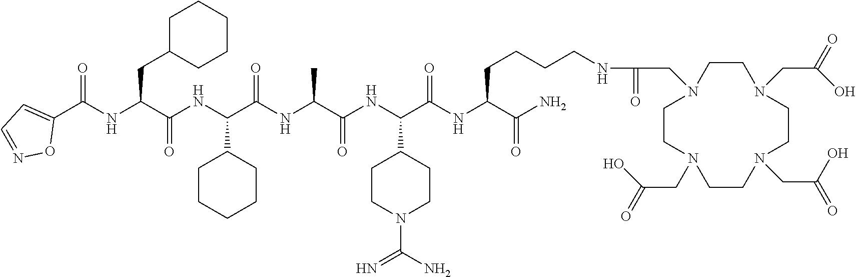 [SMILES: C[C@H](NC(=O)[C@@H](NC(=O)[C@H](CC1CCCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₄H₈₉N₁₅O₁₄: 1171.67, found [M+3H]⁺: 391.85.

### Example 223: Compound 223 (SEQ ID NO: 375)

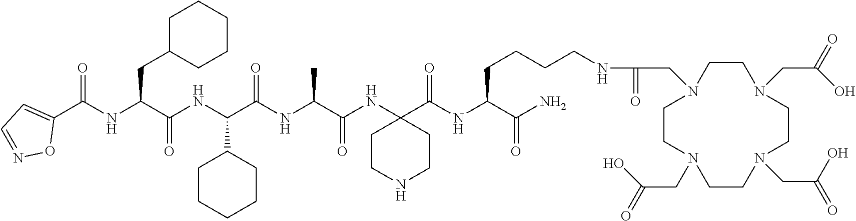 [SMILES: C[C@H](NC(=O)[C@@H](NC(=O)[C@H](CC1CCCCC1)NC(=O)c1ccno1)C1CCCCC1)C(=O)NC1(C(=O)N[C@@H](CCCCNC(=O)CN2CCN(CC(=O)O)CCN(CC(=O)O)CCN(CC(=O)O)CC2)C(N)=O)CCNCC1]

MS: Calc'd for C₅₂H₃₅N₁₃O₁₄: 1115.63, found [M+2H]²⁺: 559.2.

### Example 224: Compound 224 (SEQ ID NO: 376)

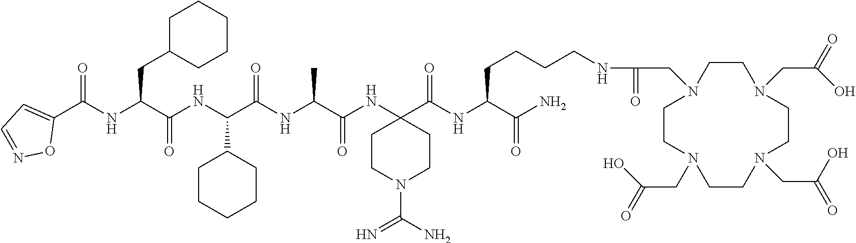 [SMILES: C[C@H](NC(=O)[C@@H](NC(=O)[C@H](CC1CCCCC1)NC(=O)c1ccno1)C1CCCCC1)C(=O)NC1(C(=O)N[C@@H](CCCCNC(=O)CN2CCN(CC(=O)O)CCN(CC(=O)O)CCN(CC(=O)O)CC2)C(N)=O)CCN(C(=N)N)CC1]

MS: Calc'd for C₅₃H₈₇N₁₅O₁₄: 1157.65, found [M+2H]²⁺: 580.2.

### Example 225: Compound 225 (SEQ ID NO: 377)

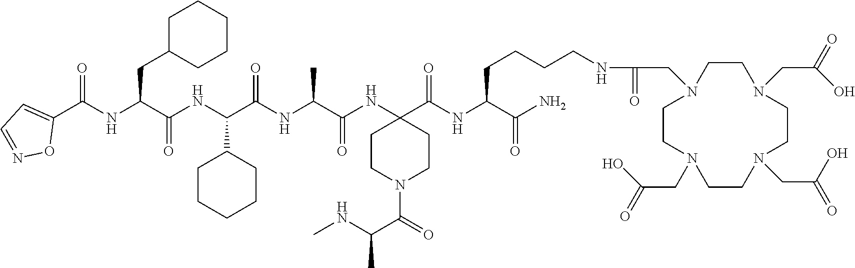 [SMILES: CN[C@H](C)C(=O)N1CCC(NC(=O)[C@H](C)NC(=O)[C@@H](NC(=O)[C@H](CC2CCCCC2)NC(=O)c2ccno2)C2CCCCC2)(C(=O)N[C@@H](CCCCNC(=O)CN2CCN(CC(=O)O)CCN(CC(=O)O)CCN(CC(=O)O)CC2)C(N)=O)CC1]

MS: Calc'd for C₅₆H₉₂N₁₄O₁₅: 1200.69, found [M+2H]²⁺: 601.6.

### Example 226: Compound 226 (SEQ ID NO: 378)

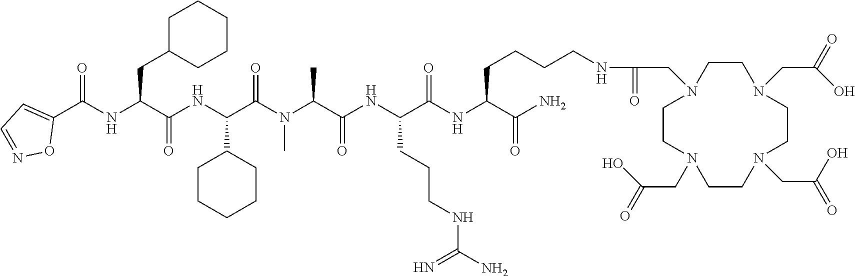 [SMILES: C[C@@H](C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O)N(C)C(=O)[C@@H](NC(=O)[C@H](CC1CCCCC1)NC(=O)c1ccno1)C1CCCCC1]

MS: Calc'd for C₅₃H₈₉N₁₅O₁₄: 1159.67, found [M+2H]²⁺: 581.2.

### Example 227: Compound 227 (SEQ ID NO: 379)

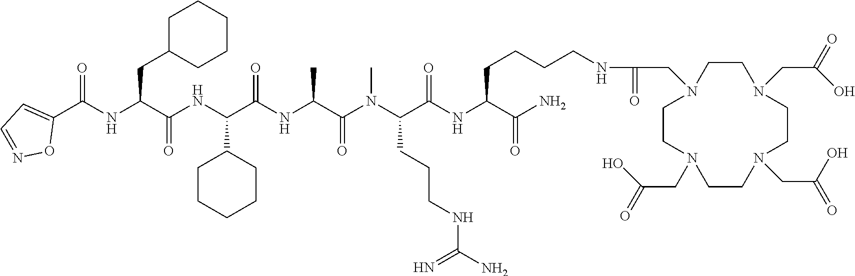 [SMILES: C[C@H](NC(=O)[C@@H](NC(=O)[C@H](CC1CCCCC1)NC(=O)c1ccno1)C1CCCCC1)C(=O)N(C)[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₃H₈₉N₁₅O₁₄: 1159.67, found [M+2H]²⁺: 581.25.

### Example 228: Compound 228 (SEQ ID NO: 380)

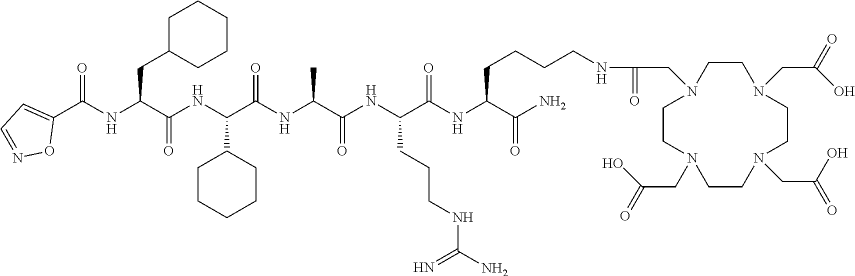 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₈₇N₁₅O₁₄: 1146.66, found [M+2H]²⁺: 574.15.

### Example 229: Compound 229 (SEQ ID NO: 381)

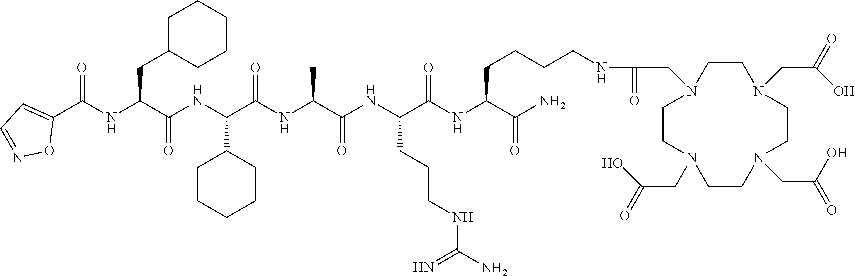 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₈₇N₁₅O₁₄: 1145.66, found [M+2H]²⁺: 574.15.

### Example 230: Compound 230 (SEQ ID NO: 382)

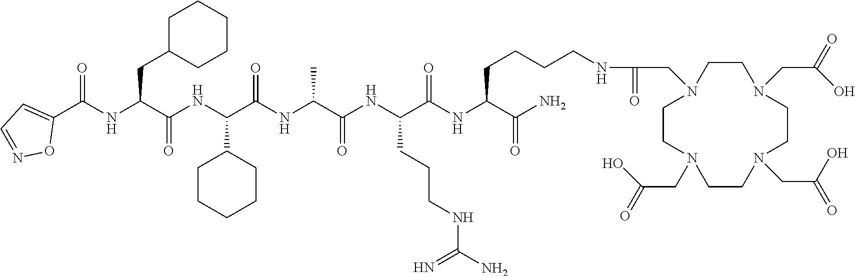 [SMILES: C[C@@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₈₇N₁₅O₁₄: 1145.66, found [M+2H]²⁺: 574.2.

### Example 231: Compound 231 (SEQ ID NO: 383)

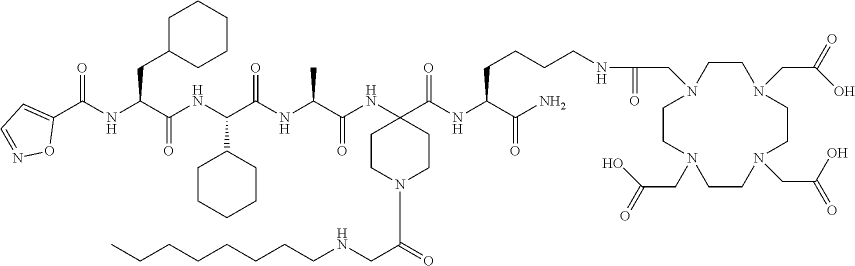 [SMILES: CCCCCCCCNCC(=O)N1CCC(NC(=O)[C@H](C)NC(=O)[C@@H](NC(=O)[C@H](CC2CCCCC2)NC(=O)c2ccno2)C2CCCCC2)(C(=O)N[C@@H](CCCCNC(=O)CN2CCN(CC(=O)O)CCN(CC(=O)O)CCN(CC(=O)O)CC2)C(N)=O)CC1]

MS: Calc'd for C₆₂104N₁₄O₁₅: 1284.78, found [M+2H]²⁺: 643.6.

### Example 232: Compound 232 (SEQ ID NO: 384)

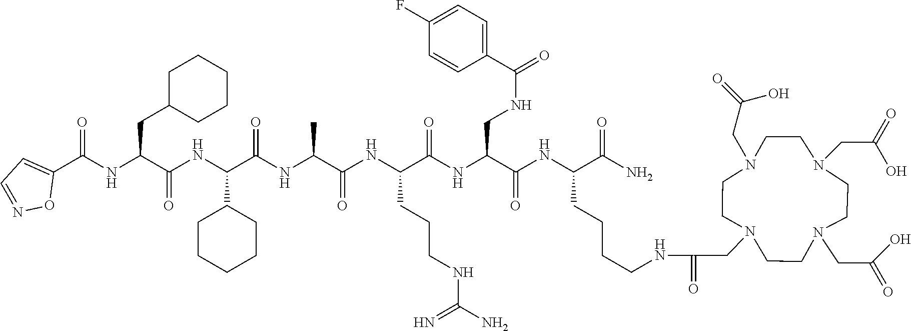 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O]

MS: Calc'd for C₆₂H₉₆N₁₇O₁₆1353.72, found [M+3H]³⁺: 452.55.

### Example 233: Compound 233 (SEQ ID NO: 385)

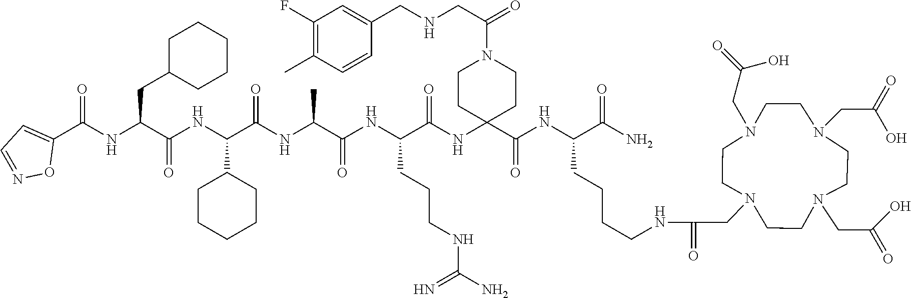 [SMILES: Cc1ccc(CNCC(=O)N2CCC(NC(=O)[C@H](CCCNC(=N)N)NC(=O)[C@H](C)NC(=O)[C@@H](NC(=O)[C@H](CC3CCCCC3)NC(=O)c3ccno3)C3CCCCC3)(C(=O)N[C@@H](CCCCNC(=O)CN3CCN(CC(=O)O)CCN(CC(=O)O)CCN(CC(=O)O)CC3)C(N)=O)CC2)cc1F]

MS: Calc'd for C₆₈H₁₀₇N₁₈O₁₆: 1450.81, found [M+H]⁺: 1451.3.

### Example 234: Compound 234 (SEQ ID NO: 386)

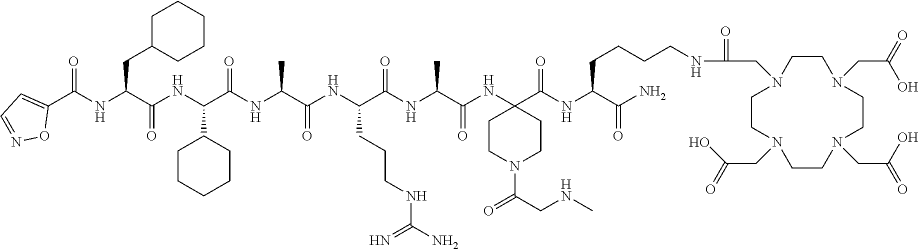 [SMILES: CNCC(=O)N1CCC(NC(=O)[C@H](C)NC(=O)[C@H](CCCNC(=N)N)NC(=O)[C@H](C)NC(=O)[C@@H](NC(=O)[C@H](CC2CCCCC2)NC(=O)c2ccno2)C2CCCCC2)(C(=O)N[C@@H](CCCCNC(=O)CN2CCN(CC(=O)O)CCN(CC(=O)O)CCN(CC(=O)O)CC2)C(N)=O)CC1]

MS: Calc'd for C₆₄H₁₀₇N₁₉O₁₇: 1413.81, found [M+3H]³⁺: 472.5.

### Example 235: Compound 235

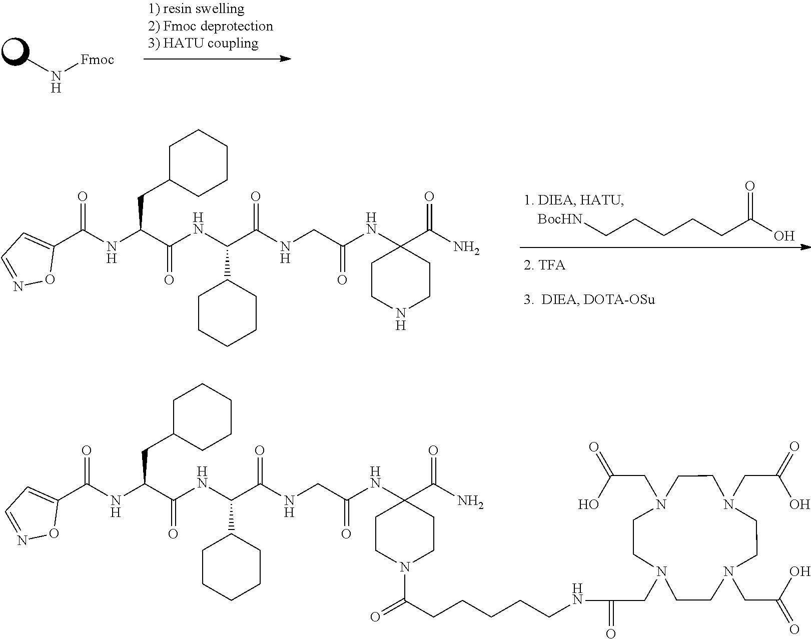

Steps 1-5: N—((S)-1-(((S)-2-((2-((4-carbamoylpiperidin-4-yl)amino)-2-oxoethyl)amino)-1-cyclohexyl-2-oxoethyl)amino)-3-cyclohexyl-1-oxopropan-2-yl) isoxazole-5-carboxamide was prepared by standard Fmoc-based SPPS procedures from Sieber resin (General SPPS procedure 1-3).

Step 6: A mixture of 6-((tert-butoxycarbonyl)amino) hexanoic acid (31 mg, 0.98 Eq, 0.13 mmol), HATU (52 mg, 1.0 Eq, 0.14 mmol), DIEA (53 mg, 71 μL, 3.0 Eq, 0.41 mmol) and DMF (1 mL) was stirred at 20° C. for 10 minutes, then N—((S)-1-(((S)-2-((2-((4-carbamoylpiperidin-4-yl)amino)-2-oxoethyl)amino)-1-cyclohexyl-2-oxoethyl)amino)-3-cyclohexyl-1-oxopropan-2-yl) isoxazole-5-carboxamide (80 mg, 1 Eq, 0.14 mmol) was added and the reaction mixture was stirred at 25° C. for an additional 1 hour. The mixture was directly purified by MPLC to afford tert-butyl (6-(4-carbamoyl-4-(2-((S)-2-cyclohexyl-2-((S)-3-cyclohexyl-2-(isoxazole-5-carboxamido) propanamido) acetamido) acetamido) piperidin-1-yl)-6-oxohexyl) carbamate (45 mg, 56 μmol, 41%) as a yellow oil. [M+H]⁺=801.6.

Step 7: To a mixture of tert-butyl (6-(4-carbamoyl-4-(2-((S)-2-cyclohexyl-2-((S)-3-cyclohexyl-2-(isoxazole-5-carboxamido) propanamido) acetamido) acetamido) piperidin-1-yl)-6-oxohexyl) carbamate (35 mg, 1 Eq, 44 μmol) and DCM (1 mL) was added TFA (0.2 mL). The reaction mixture was stirred at 25° C. for 30 min. The mixture was concentrated under reduced pressure to provide N—((S)-1-(((S)-2-((2-((1-(6-aminohexanoyl)-4-carbamoylpiperidin-4-yl)amino)-2-oxoethyl)amino)-1-cyclohexyl-2-oxoethyl)amino)-3-cyclohexyl-1-oxopropan-2-yl) isoxazole-5-carboxamide (35 mg, 45 μmol) as a yellow crude oil, which was used directly in the next step without any purification.

Step 8: The product from Step 7 was treated with DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 219, Step 7 to provide the 2,2,2-trifluoroacetic acid salt of 2,2′,2″-(10-(2-((6-(4-carbamoyl-4-(2-((S)-2-cyclohexyl-2-((S)-3-cyclohexyl-2-(isoxazole-5-carboxamido) propanamido) acetamido) acetamido) piperidin-1-yl)-6-oxohexyl)amino)-2-oxoethyl)-1,4,7,10-tetraazacyclododecane-1,4,7-triyl)triacetic acid (16.5 mg, 13.7 μmol, 28%) as a white solid. MS: Calc'd for C₅₁H₈₂N₁₂O₁₄: 1086.61, found [M+H]⁺: 1087.4.

### Example 236: Compound 236

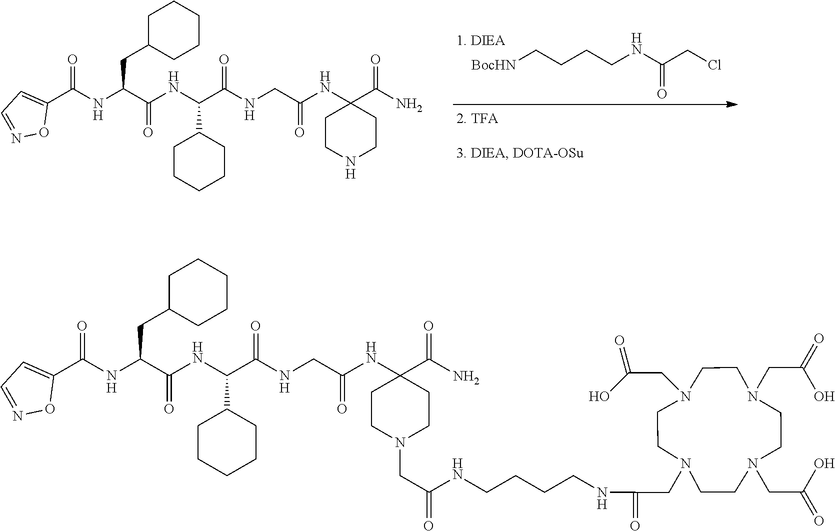 [SMILES: NC(=O)C1(NC(=O)CNC(=O)[C@@H](NC(=O)[C@H](CC2CCCCC2)NC(=O)c2ccno2)C2CCCCC2)CCNCC1 ; CC(C)(C)OC(=O)NCCCCNC(=O)CCl ; NC(=O)C1(NC(=O)CNC(=O)[C@@H](NC(=O)[C@H](CC2CCCCC2)NC(=O)c2ccno2)C2CCCCC2)CCN(CC(=O)NCCCCNC(=O)CN2CCN(CC(=O)O)CCN(CC(=O)O)CCN(CC(=O)O)CC2)CC1]

Step 1: A mixture of N—((S)-1-(((S)-2-((2-((4-carbamoylpiperidin-4-yl)amino)-2-oxoethyl)amino)-1-cyclohexyl-2-oxoethyl)amino)-3-cyclohexyl-1-oxopropan-2-yl) isoxazole-5-carboxamide (100 mg, 1 Eq, 170 μmol), DIEA (66 mg, 89 μL, 3.0 Eq, 0.51 mmol), tert-butyl (4-(2-chloroacetamido)butyl) carbamate (90 mg, 2.0 Eq, 0.34 mmol) and DMF (10 mL) was stirred at 30° C. for 16 hours. The mixture was directly purified by MPLC to afford tert-butyl (4-(2-(4-carbamoyl-4-(2-((S)-2-cyclohexyl-2-((S)-3-cyclohexyl-2-(isoxazole-5-carboxamido) propanamido) acetamido) acetamido) piperidin-1-yl) acetamido)butyl) carbamate (30 mg, 22 μmol, 13%) as a yellow oil. [M+H]⁺=816.4.

Steps 2-3: tert-Butyl (4-(2-(4-carbamoyl-4-(2-((S)-2-cyclohexyl-2-((S)-3-cyclohexyl-2-(isoxazole-5-carboxamido) propanamido) acetamido) acetamido) piperidin-1-yl) acetamido)butyl) carbamate was treated with TFA then DIEA and 2,2′,2″-(10-(2-((2,5-dioxopyrrolidin-1-yl)oxy)-2-oxoethyl)-1,4,7,10-tetraazacyclododecane-1,4,7-triyl)triacetic acid in a manner similar to Example 235, Steps 7-8 to provide the 2,2,2-trifluoroacetic acid salt of 2,2′,2″-(10-(2-((4-(2-(4-carbamoyl-4-(2-((S)-2-cyclohexyl-2-((S)-3-cyclohexyl-2-(isoxazole-5-carboxamido) propanamido) acetamido) acetamido) piperidin-1-yl) acetamido)butyl)amino)-2-oxoethyl)-1,4,7,10-tetraazacyclododecane-1,4,7-triyl)triacetic acid (3.7 mg, 3.0 μmol, 23%) as a white solid. MS: Calc'd for C₅₁H₂₃N₁₃O₁₄: 1101.61, found [M+H]⁺: 1102.5.

### Example 237: Compound 237 (SEQ ID NO: 389)

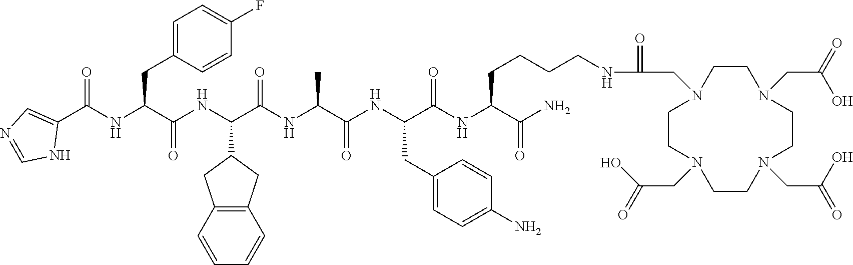 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(N)=O]

MS: Calc'd for C₅₈H₇₇N₁₄O₁₃: 1196.58, found [M+3H]⁺: 400.2.

### Example 238: Compound 238 (SEQ ID NO: 390)

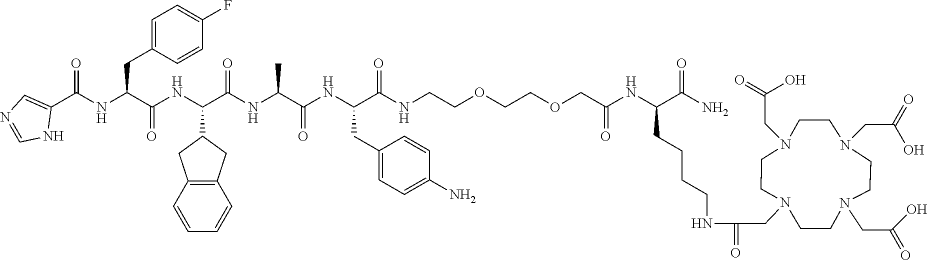 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@@H](Cc1ccc(N)cc1)C(=O)NCCOCCOCC(=O)N[C@H](CCCCNC(=O)CN1CCN(CC(=O)O)CCN(CC(=O)O)CCN(CC(=O)O)CC1)C(N)=O]

MS: Calc'd for C₆₄H₈₂N₁₅O₁₆: 1341.65, found [M+3H]³⁺: 448.5.

### Synthesis of Compound 238-In (SEQ ID NO: 391)

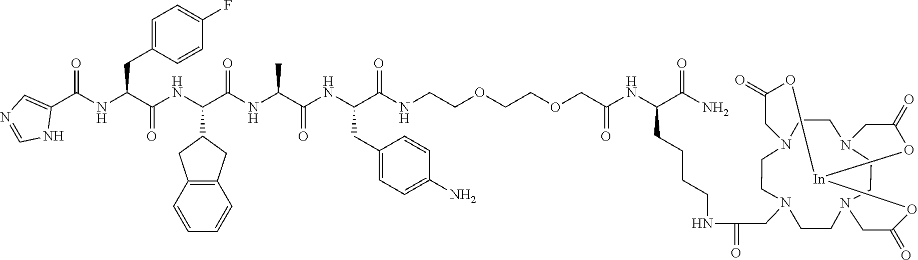

MS: Calc'd for C₆₄H₈₅InN₁₅O₁₆: 1453.53, found [M+3H]³⁺: 485.85.

### Example 239: Compound 239 (SEQ ID NO: 392)

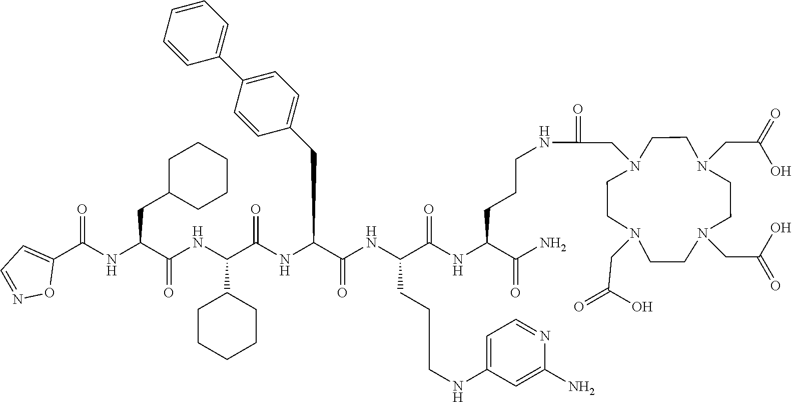 [SMILES: NC(=O)[C@H](CCCNC(=O)CN1CCN(CC(=O)O)CCN(CC(=O)O)CCN(CC(=O)O)CC1)NC(=O)[C@H](CCCNc1ccnc(N)c1)NC(=O)[C@H](Cc1ccc(-c2ccccc2)cc1)NC(=O)[C@@H](NC(=O)[C@H](CC1CCCCC1)NC(=O)c1ccno1)C1CCCCC1]

MS: Calc'd for C₆₇H₉₅N₁₅O₁₄: 1333.82, found [M+H]⁺: 1334.65.

### Example 240: Compound 240

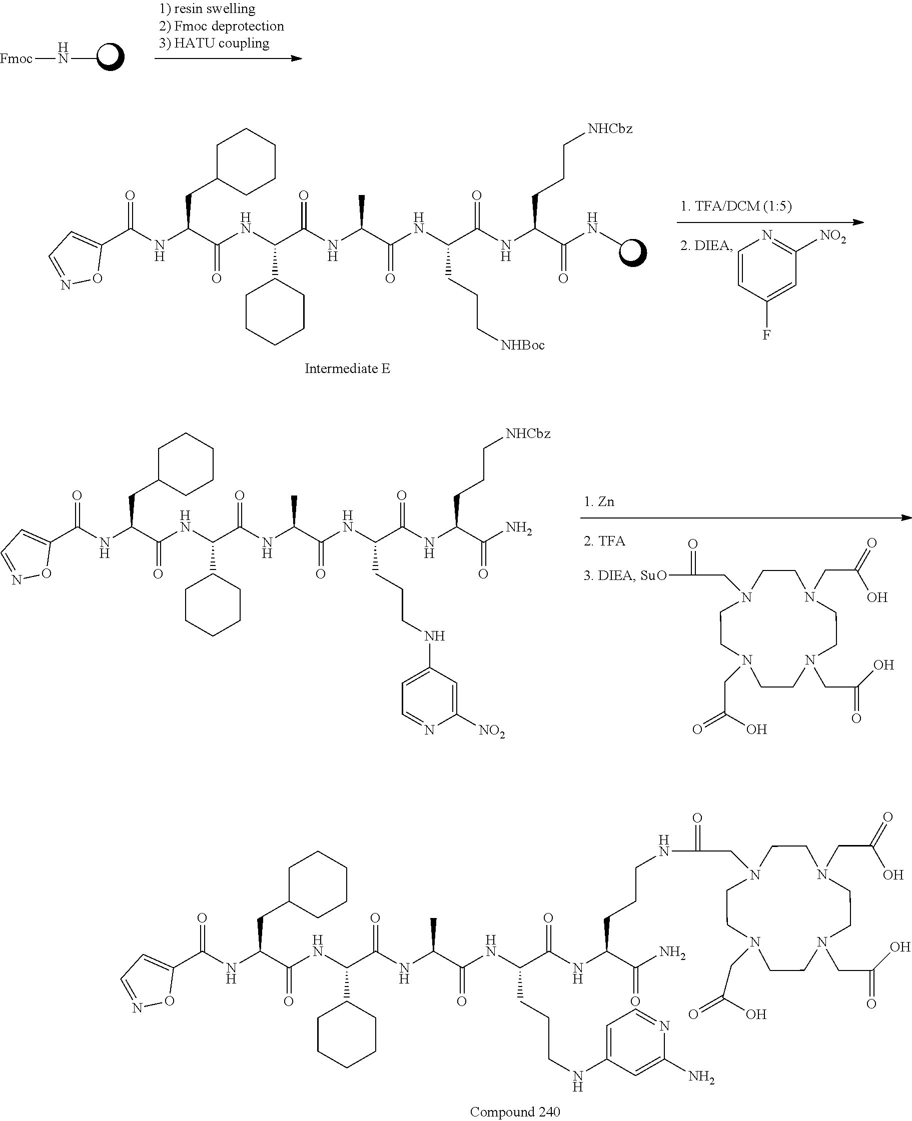

Steps 1-3: Intermediate E on resin was prepared by standard Fmoc-based SPPS procedures from Sieber resin (General SPPS procedure 1-3).

Step 4: Intermediate E was cleaved from the resin with the solution (v/v TFA/DCM=1:5) for 20 min at 30° C. The mixture was filtered, and the filtrate was concentrated under reduced pressure. After the residue was precipitated with cold ether (6 mL), the precipitates were collected by centrifugation, three repetitions of centrifuging are required. The crude product was dissolved in DMF (5 mL) and purified by prep-HPLC with the following conditions: Column: SunFire prep OBD 19*150 mm 5 um; Mobile Phase A: Water (0.05% TFA); Mobile Phase B: ACN; Gradient: 25% B to 65% B in 8 min; Flow rate: 20 mL/min; Wavelength: 220 nm. The collected fractions were dried by lyophilization. This resulted in benzyl ((3S,6S,9S,12S,15S)-12-(3-aminopropyl)-15-carbamoyl-6-cyclohexyl-3-(cyclohexylmethyl)-1-(isoxazol-5-yl)-9-methyl-1,4,7,10,13-pentaoxo-2,5,8,11,14-pentaazaoctadecan-18-yl) carbamate (100 mg, 119 μmol, 17.1%) as a white solid. [M+H]⁺=838.4.

Step 5: A mixture of benzyl ((3S,6S,9S,12S,15S)-12-(3-aminopropyl)-15-carbamoyl-6-cyclohexyl-3-(cyclohexylmethyl)-1-(isoxazol-5-yl)-9-methyl-1,4,7,10,13-pentaoxo-2,5,8,11,14-pentaazaoctadecan-18-yl) carbamate (100 mg, 1 Eq, 119 μmol), DIEA (50 mg, 67 μL, 3.2 Eq, 0.39 mmol), 4-fluoro-2-nitropyridine (80 mg, 4.7 Eq, 0.56 mmol) and DMF (2 mL) was stirred at 35° C. for 4 hours. The crude product was purified by Prep-HPLC to afford benzyl ((3S,6S,9S,12S,15S)-15-carbamoyl-6-cyclohexyl-3-(cyclohexylmethyl)-1-(isoxazol-5-yl)-9-methyl-12-(3-((2-nitropyridin-4-yl)amino) propyl)-1,4,7,10,13-pentaoxo-2,5,8,11,14-pentaazaoctadecan-18-yl) carbamate (80 mg, 83 μmol, 70%) as a white solid. [M+H]⁺=960.6.

Step 6: A mixture of benzyl ((3S,6S,9S,12S,15S)-15-carbamoyl-6-cyclohexyl-3-(cyclohexylmethyl)-1-(isoxazol-5-yl)-9-methyl-12-(3-((2-nitropyridin-4-yl)amino) propyl)-1,4,7,10,13-pentaoxo-2,5,8,11,14-pentaazaoctadecan-18-yl) carbamate (80 mg, 1 Eq, 83 μmol), zinc (50 mg, 9.2 Eq, 0.76 mmol) and AcOH (5 mL) was stirred at 25° C. for 1 hour. The mixture was directly purified by MPLC to afford benzyl ((3S,6S,9S,12S,15S)-12-(3-((2-aminopyridin-4-yl)amino) propyl)-15-carbamoyl-6-cyclohexyl-3-(cyclohexylmethyl)-1-(isoxazol-5-yl)-9-methyl-1,4,7,10,13-pentaoxo-2,5,8,11,14-pentaazaoctadecan-18-yl) carbamate (60 mg, 65 μmol, 77%) as a yellow oil. [M+H]⁺=930.7.

Step 7: A mixture of benzyl ((3S,6S,9S,12S,15S)-12-(3-((2-aminopyridin-4-yl)amino) propyl)-15-carbamoyl-6-cyclohexyl-3-(cyclohexylmethyl)-1-(isoxazol-5-yl)-9-methyl-1,4,7,10,13-pentaoxo-2,5,8,11,14-pentaazaoctadecan-18-yl) carbamate (60 mg, 1 Eq, 65 μmol) and TFA (2 mL) was stirred at 25° C. for 1 hour. The mixture was directly purified by MPLC to provide N-((2S,5S,8S,11S,14S)-17-amino-11-(3-((2-aminopyridin-4-yl)amino) propyl)-14-carbamoyl-1,5-dicyclohexyl-8-methyl-3,6,9,12-tetraoxo-4,7,10,13-tetraazaheptadecan-2-yl) isoxazole-5-carboxamide (45 mg, 57 μmol, 88%) as a yellow oil. [M+H]⁺=796.7.

Step 8: A mixture of N-((2S,5S,8S,11S,14S)-17-amino-11-(3-((2-aminopyridin-4-yl)amino) propyl)-14-carbamoyl-1,5-dicyclohexyl-8-methyl-3,6,9,12-tetraoxo-4,7,10,13-tetraazaheptadecan-2-yl) isoxazole-5-carboxamide (12 mg, 1 Eq, 15 μmol), DIEA (6 mg, 8 μL, 3 Eq, 0.05 mmol) and DMF (1 mL) was stirred at 25° C. for 1 hour. The mixture was directly purified by Prep-HPLC to provide the trifluoroacetic acid salt of 2,2′,2″-(10-((3S,6S,9S,12S,15S)-12-(3-((2-aminopyridin-4-yl)amino) propyl)-15-carbamoyl-6-cyclohexyl-3-(cyclohexylmethyl)-1-(isoxazol-5-yl)-9-methyl-1,4,7,10,13,20-hexaoxo-2,5,8,11,14,19-hexaazahenicosan-21-yl)-1,4,7,10-tetraazacyclododecane-1,4,7-triyl)triacetic acid (11.9 mg, 9.18 μmol, 61%) as a white solid. MS: Calc'd for C₅₅H₈₇N₁₅O₁₄: 1181.66, found [M+H]⁺: 1182.5.

### Synthesis of Compound 240-In

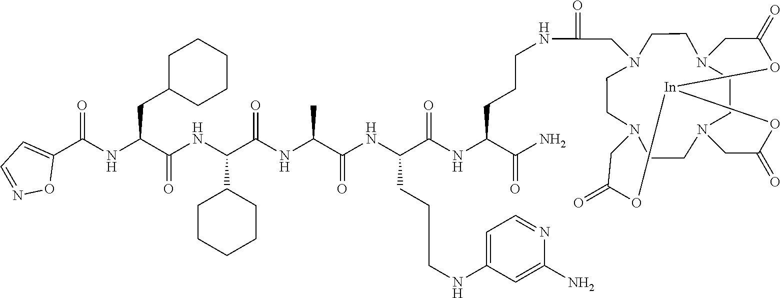

A mixture of 2,2′,2″-(10-((3S,6S,9S,12S,15S)-12-(3-((2-aminopyridin-4-yl)amino) propyl)-15-carbamoyl-6-cyclohexyl-3-(cyclohexylmethyl)-1-(isoxazol-5-yl)-9-methyl-1,4,7,10,13,20-hexaoxo-2,5,8,11,14,19-hexaazahenicosan-21-yl)-1,4,7,10-tetraazacyclododecane-1,4,7-triyl)triacetic acid (40 mg, 1 Eq, 34 μmol), indium trichloride (37 mg, 11 μL, 4.9 Eq, 0.17 mmol) and NaOAc—AcOH (1 mL) was stirred at 80° C. for 2 hours. The crude mixture was directly purified by Prep-HPLC to provide the product (34.7 mg, 24.6 μmol, 73%) as a white solid. MS: Calc'd for C₅₅H₃₄InN₁₅O₁₄: 1293.54, found [M+H]⁺: 1294.9.

### Synthesis of Compound 240-Lu

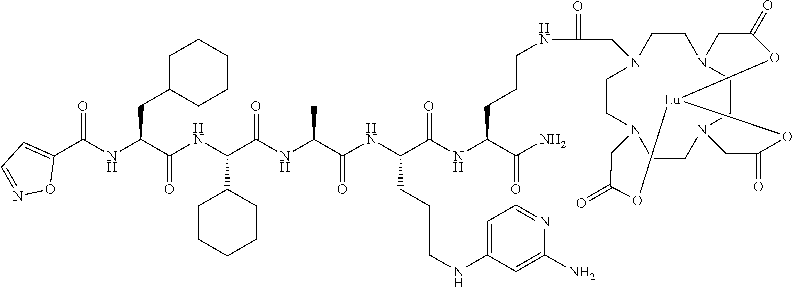

A solution of 2,2′,2″-(10-((3S,6S,9S,12S,15S)-12-(3-((2-aminopyridin-4-yl)amino) propyl)-15-carbamoyl-6-cyclohexyl-3-(cyclohexylmethyl)-1-(isoxazol-5-yl)-9-methyl-1,4,7,10,13,20-hexaoxo-2,5,8,11,14,19-hexaazahenicosan-21-yl)-1,4,7,10-tetraazacyclododecane-1,4,7-triyl)triacetic acid (20 mg, 0.017 mmol) and trichlorolutetium (20.7 mg, 0.074 mmol) in sodium acetate buffer pH=5.6 (1 mL) was stirred at room temperature for 2 h. The crude product was purified by Prep-HPLC to afford the product (6.3 mg, 26%) as a white solid. MS: Calc'd for C₅₅H₈₄LuN₁₅O₁₄: 1353.57, found [M+H]⁺: 1354.5.

### Synthesis of Compound 240-Ga

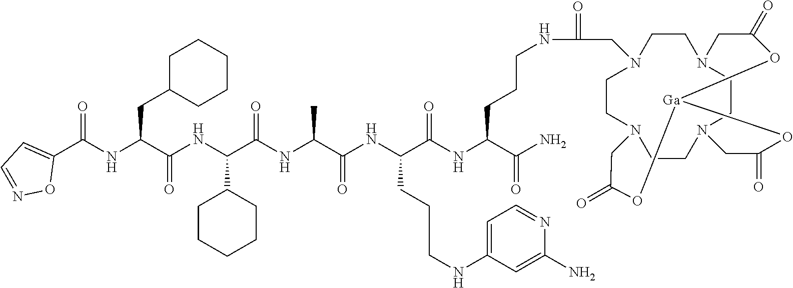

A solution of 2,2′,2″-(10-((3S,6S,9S,12S,15S)-12-(3-((2-aminopyridin-4-yl)amino) propyl)-15-carbamoyl-6-cyclohexyl-3-(cyclohexylmethyl)-1-(isoxazol-5-yl)-9-methyl-1,4,7,10,13,20-hexaoxo-2,5,8,11,14,19-hexaazahenicosan-21-yl)-1,4,7,10-tetraazacyclododecane-1,4,7-triyl)triacetic acid (30 mg, 0.025 mmol) and trichlorogallane (19.4 mg, 0.109 mmol) in sodium acetate buffer pH=5.6 (1 mL) was stirred at room temperature overnight. The crude product was purified by Prep-HPLC to afford the product (10.7 mg, 31.4%) as a light yellow solid. MS: Calc'd for C₅₅H₂₄GaN₁₅O₁₄: 1247.56, found [M+2H]²⁺: 625.45.

### Example 241: Compound 241

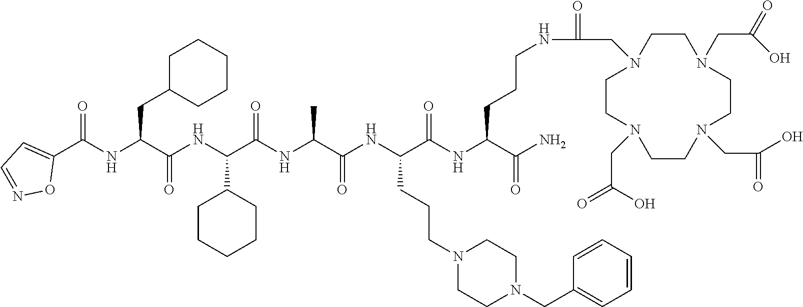 [SMILES: C[C@H](NC(=O)[C@@H](NC(=O)[C@H](CC1CCCCC1)NC(=O)c1ccno1)C1CCCCC1)C(=O)N[C@@H](CCCN1CCN(Cc2ccccc2)CC1)C(=O)N[C@@H](CCCNC(=O)CN1CCN(CC(=O)O)CCN(CC(=O)O)CCN(CC(=O)O)CC1)C(N)=O]

### Synthesis of (S)-2-((((9H-fluoren-9-yl) methoxy) carbonyl)amino)-5-(4-benzylpiperazin-1-yl) pentanoic acid

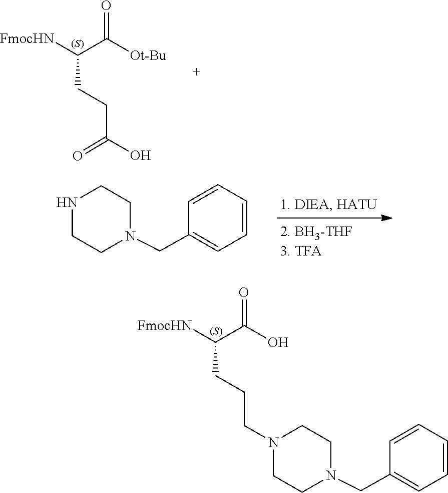

Step 1: A mixture of(S)-4-((((9H-fluoren-9-yl) methoxy) carbonyl)amino)-5-(tert-butoxy)-5-oxopentanoic acid (1.0 g, 1 Eq, 2.4 mmol), HATU (1.1 g, 1.2 Eq, 2.9 mmol), DIEA (0.91 g, 1.2 mL, 3.0 Eq, 7.0 mmol) and DMF (10 mL) was stirred at 20° C. for 10 minutes, then 1-benzylpiperazine (0.5 g, 1 Eq, 3 mmol) was added and the reaction mixture was stirred at 25° C. for an additional 1 hour. The mixture was directly purified by MPLC to afford tert-butyl(S)-2-((((9H-fluoren-9-yl) methoxy) carbonyl)amino)-5-(4-benzylpiperazin-1-yl)-5-oxopentanoate (1.5 g, 2.2 mmol, 93%, 85% Purity) as a yellow oil. [M+H]⁺=584.3.

Step 2: To a mixture of tert-butyl(S)-2-((((9H-fluoren-9-yl) methoxy) carbonyl)amino)-5-(4-benzylpiperazin-1-yl)-5-oxopentanoate (800 mg, 1 Eq, 1.37 mmol) and THF (10 mL), was added a solution of borane tetrahydrofuran complex (353 mg, 396 μL, 3.00 Eq, 4.11 mmol) dropwise at 0° C. over 5 min. The reaction mixture was stirred at 25° C. for 1 hour. The mixture was directly purified by MPLC to afford tert-butyl(S)-2-((((9H-fluoren-9-yl) methoxy) carbonyl)amino)-5-(4-benzylpiperazin-1-yl) pentanoate (360 mg, 632 μmol, 46.1%) as a yellow oil. [M+H]⁺=570.5.

Step 3: To a mixture of tert-butyl(S)-2-((((9H-fluoren-9-yl) methoxy) carbonyl)amino)-5-(4-benzylpiperazin-1-yl) pentanoate (360 mg, 1 Eq, 632 μmol) and DCM (3 mL) was added TFA (1 mL). The reaction mixture was stirred at 25° C. for 30 min. The mixture was concentrated under reduced pressure. The crude product was purified by Prep-HPLC to provide(S)-2-((((9H-fluoren-9-yl) methoxy) carbonyl)amino)-5-(4-benzylpiperazin-1-yl) pentanoic acid (300 mg, 584 μmol, 92.4%) as a white solid. MS: Calc'd for C₃₁H₃₅N₃O₄: 513.26, found [M+H]⁺: 514.5.

Compound 241 was synthesized according to the generic procedures described above to provide the product. MS: Calc'd for C₆₁H₉₆N₁₄O₁₄: 1248.72, found [M+H]⁺: 1249.9.

### Example 242: Compound 242 (SEQ ID NO: 395)

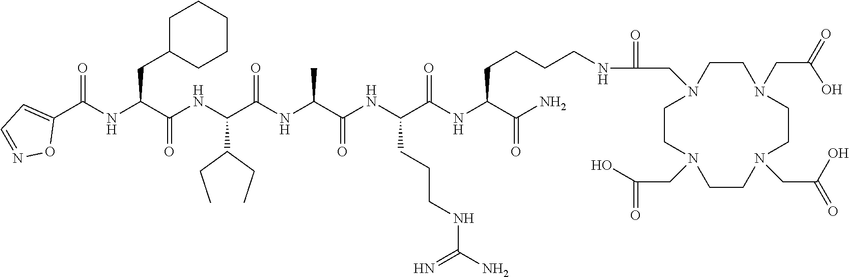 [SMILES: CCC(CC)[C@H](NC(=O)[C@H](CC1CCCCC1)NC(=O)c1ccno1)C(=O)N[C@@H](C)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₁H₈₇N₁₅O₁₄: 1133.66, found [M+2H]²⁺: 568.2.

### Example 243: Compound 243 (SEQ ID NO: 396)

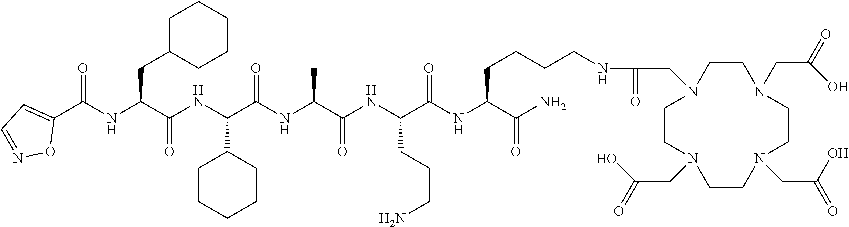 [SMILES: C[C@H](NC(=O)[C@@H](NC(=O)[C@H](CC1CCCCC1)NC(=O)c1ccno1)C1CCCCC1)C(=O)N[C@@H](CCCN)C(=O)N[C@@H](CCCCNC(=O)CN1CCN(CC(=O)O)CCN(CC(=O)O)CCN(CC(=O)O)CC1)C(N)=O]

MS: Calc'd for C₅₁H₈₅N₁₃O₁₄: 1103.63, found [M+2H]²⁺: 553.1.

### Example 244: Compound 244 (SEQ ID NO: 397)

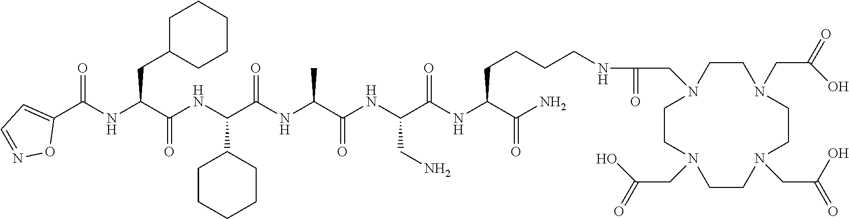 [SMILES: C[C@H](NC(=O)[C@@H](NC(=O)[C@H](CC1CCCCC1)NC(=O)c1ccno1)C1CCCCC1)C(=O)N[C@@H](CN)C(=O)N[C@@H](CCCCNC(=O)CN1CCN(CC(=O)O)CCN(CC(=O)O)CCN(CC(=O)O)CC1)C(N)=O]

MS: Calc'd for C₄₉H₃₁N₁₃O₁₄: 1075.60, found [M+2H]²⁺: 539.2.

### Example 245: Compound 245 (SEQ ID NO: 398)

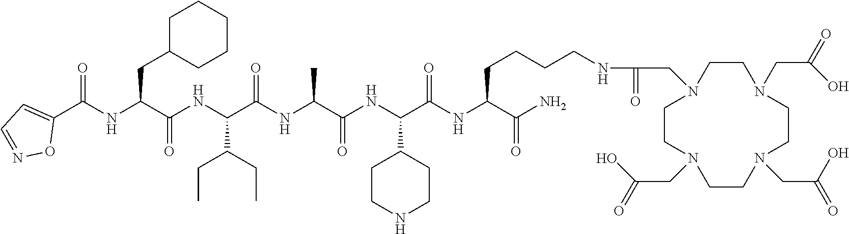 [SMILES: CCC(CC)[C@H](NC(=O)[C@H](CC1CCCCC1)NC(=O)c1ccno1)C(=O)N[C@@H](C)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₅₂H₈₇N₁₃O₁₄: 1117.65, found [M+2H]²⁺: 560.2.

### Example 246: Compound 246 (SEQ ID NO: 399)

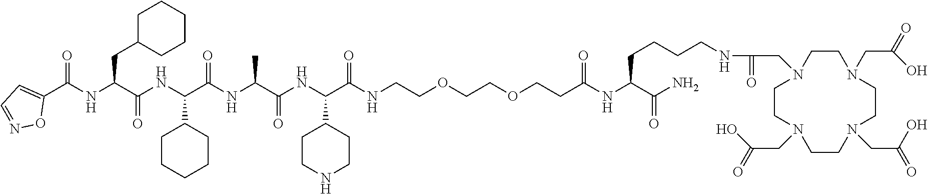 [SMILES: C[C@H](NC(=O)[C@@H](NC(=O)[C@H](CC1CCCCC1)NC(=O)c1ccno1)C1CCCCC1)C(=O)N[C@H](C(=O)NCCOCCOCCC(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₆₀H₁₀₀N₁₄O₁₇: 1288.74, found [M+2H]²⁺: 645.8.

### Example 247: Compound 247 (SEQ ID NO: 400)

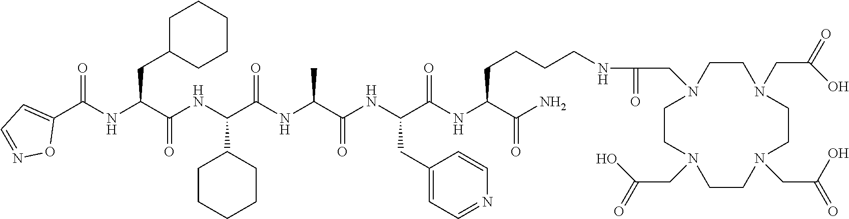 [SMILES: C[C@H](NC(=O)[C@@H](NC(=O)[C@H](CC1CCCCC1)NC(=O)c1ccno1)C1CCCCC1)C(=O)N[C@@H](Cc1ccncc1)C(=O)N[C@@H](CCCCNC(=O)CN1CCN(CC(=O)O)CCN(CC(=O)O)CCN(CC(=O)O)CC1)C(N)=O]

MS: Calc'd for C₅₄H₈₃N₁₃O₁₄: 1137.62, found [M+2H]²⁺: 570.2.

### Example 248: Compound 248 (SEQ ID NO: 401)

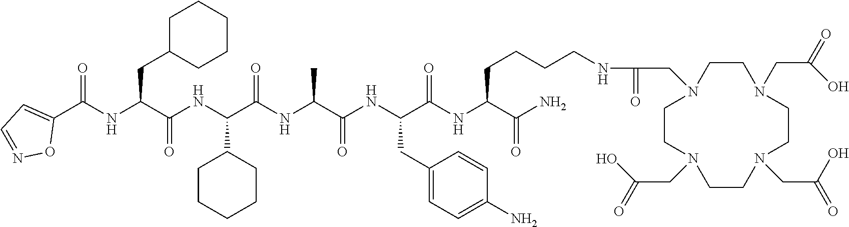 [SMILES: C[C@H](NC(=O)[C@@H](NC(=O)[C@H](CC1CCCCC1)NC(=O)c1ccno1)C1CCCCC1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(N)=O]

### Example 249: Compound 249 (SEQ ID NO: 402)

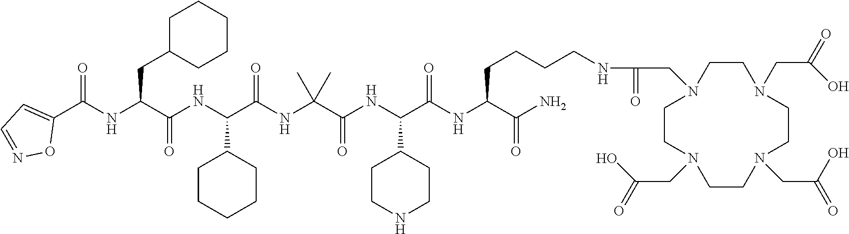 [SMILES: CC(C)(NC(=O)[C@@H](NC(=O)[C@H](CC1CCCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₅₄H₃₉N₁₃O₁₄: 1143.67, found [M+2H]²⁺: 573.2.

### Example 250: Compound 250 (SEQ ID NO: 403)

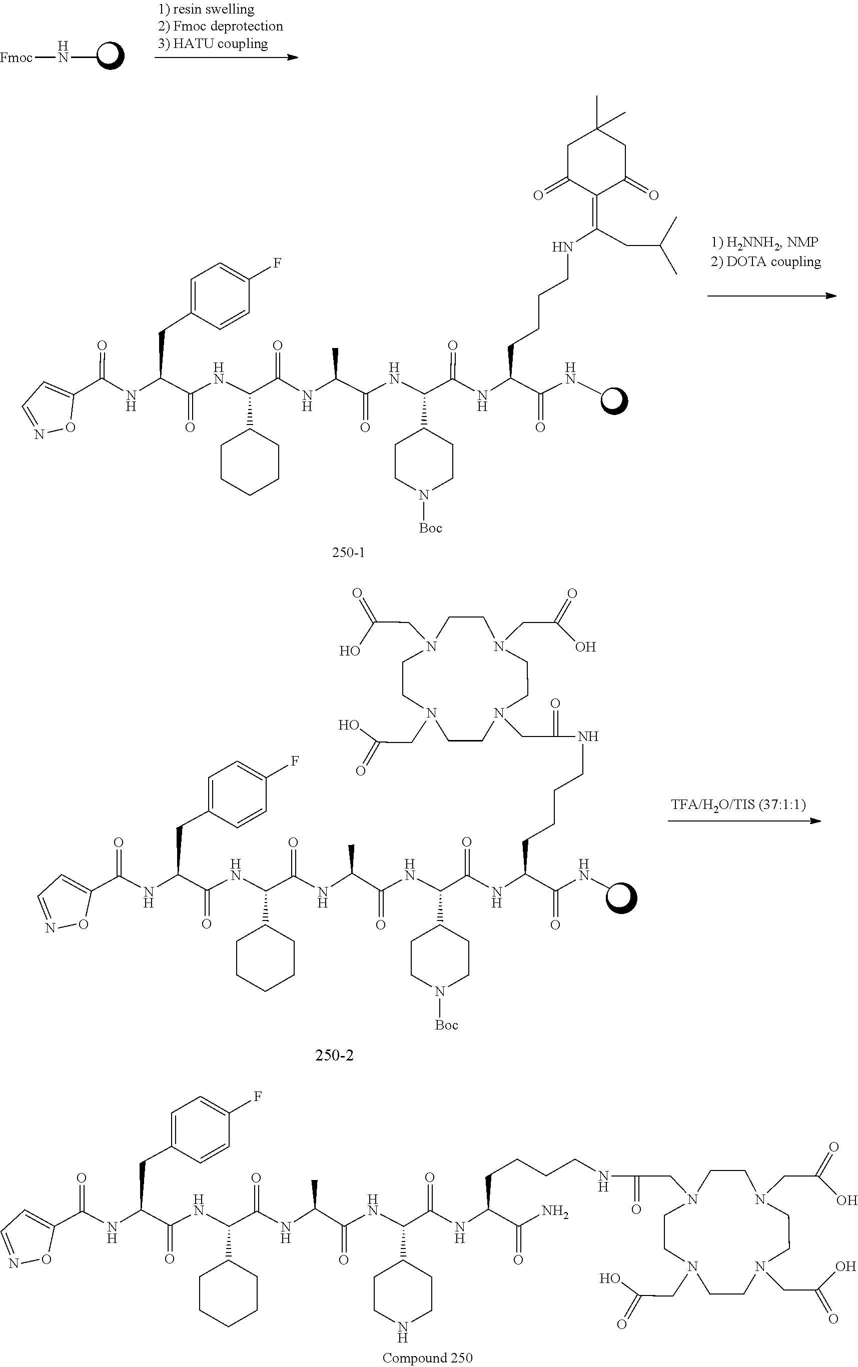

Steps 1-3: Peptidyl resin 250-1 was prepared by standard Fmoc-based SPPS procedures from Na-Fmoc-Sieber amide resin (1.60 g, 0.53 mmol/g, 0.848 mmol. Coupling conditions: 4.0 Eq amino acid building block, 4.0 Eq HATU, 8.0 Eq DIPEA in 30 mL NMP, 45 min at 30° C. under a N₂ atmosphere. Fmoc-removal conditions: 30 mL 20% piperidine in NMP, 20 min at room temperature under a N₂ atmosphere). Step 4-5: Peptidyl resin 250-1 was incubated with 2% hydrazine hydrate in NMP (30 mL). After 2 h, the solvent was removed by filtration, and the resin was washed with NMP (3×30 mL). To the resin in NMP (30 mL) were added DOTA-OSu (1.28 g, 2.544 mmol, 3.0 Eq) and DIPEA (0.66 g, 5.088 mmol, 6.0 Eq). The mixture was shaken at 30° C. under a nitrogen atmosphere for 2 h. After the solvent was removed, the resin was washed with NMP (3×30 mL) to afford peptidyl resin 250-2.

Step 5: Peptidyl resin 250-2 was treated with a mixture of TFA/H₂O/TIS (37:1:1 v/v/v, 10 mL) at room temperature. After 1 h, the mixture was filtered, and the filtrate was concentrated under reduced pressure. The residue was purified by RP-HPLC (Mobile Phase A: water (0.05% TFA), Mobile Phase B: acetonitrile). The desired fractions were combined, concentrated and lyophilized to afford Compound 250 (82.0 mg, 8.13% overall yield, 95.1 purity) as a white solid. MS: Calc'd for C₅₃H₈₀FN₁₃O₁₄: 1141.59, found [M+2H]²⁺: 572.2.

### Synthesis of Compound 250-Ga (SEQ ID NO: 694)

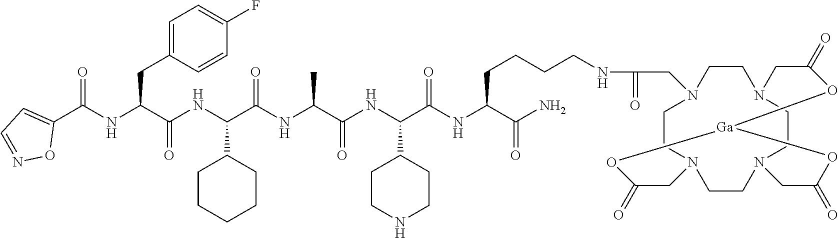

A solution of Compound 250 (15 mg, 0.013 mmol, 1.0 equiv) and gallium (III) chloride (9.94 mg, 0.056 mmol, 4.3 equiv) in sodium acetate buffer (pH 5.6, 0.25 mL) was stirred at room temperature for 2 h. The crude mixture was purified by reversed-phase HPLC. The desired fractions were combined and lyophilized to afford Compound 250-Ga as a white solid (8.8 mg, yield 53.4%, 95.7% purity). MS: Calc'd for C₅₃H₇₇FGaN₁₃O₁₄: 1207.49, found [M+2H]²⁺: 605.4.

### Synthesis of Compound 250-Lu (SEQ ID NO: 695)

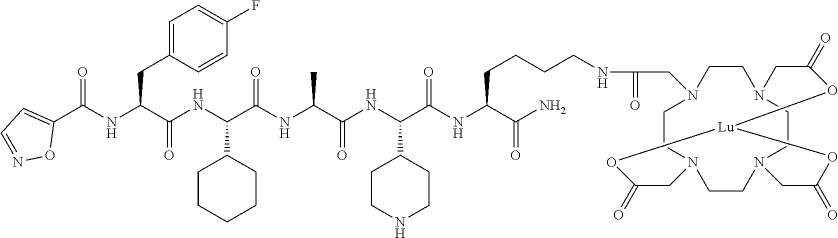

A solution of Compound 250 (15 mg, 0.013 mmol, 1.0 equiv) and lutetium (III) chloride (15.88 mg, 0.056 mmol, 4.3 equiv) in sodium acetate buffer (pH 5.6, 0.25 mL) was stirred at room temperature for 2 h. The crude mixture was purified by reversed-phase HPLC. The desired fractions were combined and lyophilized to afford Compound 250-Lu as a white solid (7.2 mg, yield 38.4%, 96.8% purity). MS: Calc'd for C₅₃H₇₇FLuN₁₃O₁₄: 1313.51, found [M+2H]²⁺: 658.0.

### Synthesis of Compound 250-In (SEQ ID NO: 696)

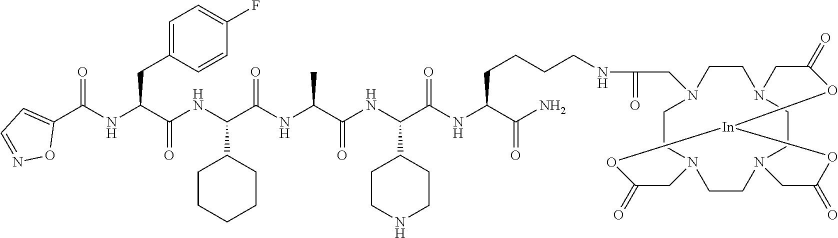

A solution of Compound 250 (20 mg, 0.018 mmol, 1.0 equiv) and indium (III) chloride (8.91 mg, 0.041 mmol, 2.3 equiv) in sodium acetate buffer (pH 5.6, 0.25 mL) was stirred at room temperature for 2 h. The crude mixture was purified by reversed-phase HPLC. The desired fractions were combined and lyophilized to afford Compound 250-In as a white solid (10.8 mg, yield 45.1%, 98.8% purity). MS: Calc'd for C₅₃H₇₇FInN₁₃O₁₄: 1253.47, found [M+2H]²⁺: 628.1.

### Example 251: Compound 251 (SEQ ID NO: 404)

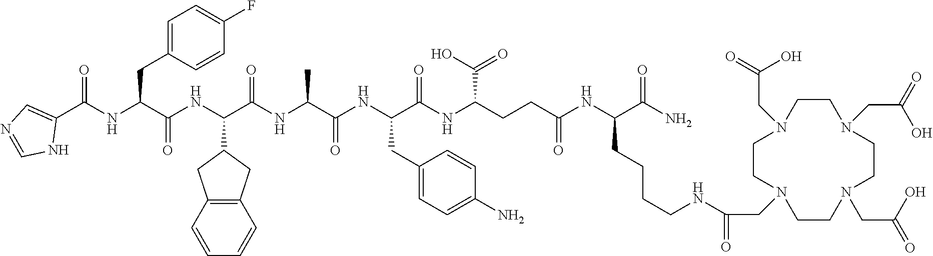 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@@H](Cc1ccc(N)cc1)C(=O)N[C@@H](CCC(=O)N[C@H](CCCCNC(=O)CN1CCN(CC(=O)O)CCN(CC(=O)O)CCN(CC(=O)O)CC1)C(N)=O)C(=O)O]

MS: Calc'd for C₆₃H₈₄FN₁₅O₁₆: 1325.62, found [M+3H]³⁺: 443.2.

### Example 252: Compound 252 (SEQ ID NO: 405)

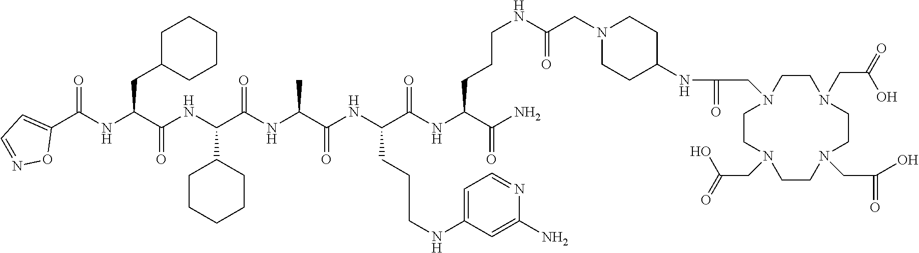 [SMILES: C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N[C@@H](CCCNC(=O)CN1CCC(NC(=O)CN2CCN(CC(=O)O)CCN(CC(=O)O)CCN(CC(=O)O)CC2)CC1)C(N)=O]

MS: Calc'd for C₆₂H₉₉N₁₇O₁₅: 1321.75, found [M+H]⁺: 1322.7.

### Example 253: Compound 253 (SEQ ID NO: 406)

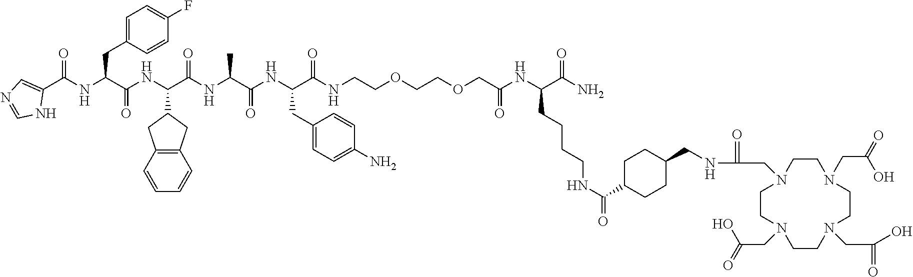

MS: Calc'd for C₂₂H₁₀₁FN₁₆O₁₇: 1480.75, found [M+2H]²⁺: 714.9.

### Example 254: Compound 254 (SEQ ID NO: 407)

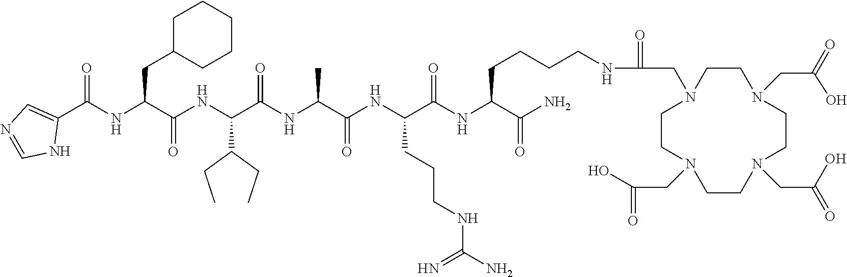 [SMILES: CCC(CC)[C@H](NC(=O)[C@H](CC1CCCCC1)NC(=O)c1cnc[nH]1)C(=O)N[C@@H](C)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₁H₈₈N₁₆O₁₃: 1132.67, found [M+2H]²⁺: 567.7.

### Example 255: Compound 255 (SEQ ID NO: 408)

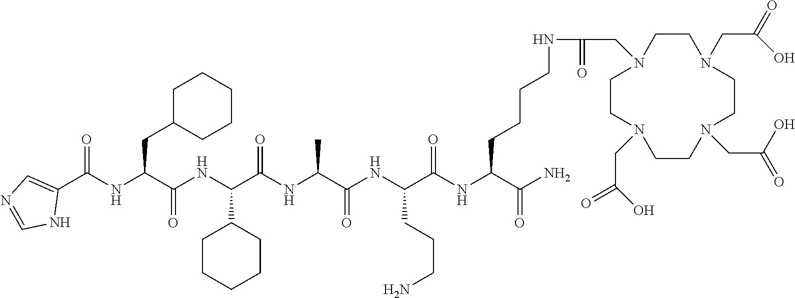 [SMILES: C[C@H](NC(=O)[C@@H](NC(=O)[C@H](CC1CCCCC1)NC(=O)c1cnc[nH]1)C1CCCCC1)C(=O)N[C@@H](CCCN)C(=O)N[C@@H](CCCCNC(=O)CN1CCN(CC(=O)O)CCN(CC(=O)O)CCN(CC(=O)O)CC1)C(N)=O]

MS: Calc'd for C₅₁H₈₆N₁₄O₁₃: 1102.65, found [M+2H]²⁺: 552.7.

### Example 256: Compound 256 (SEQ ID NO: 409)

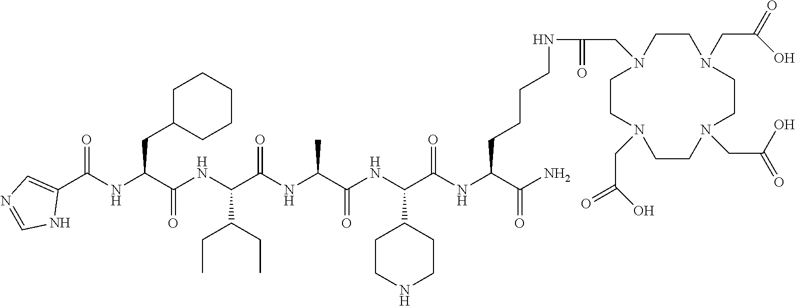 [SMILES: CCC(CC)[C@H](NC(=O)[C@H](CC1CCCCC1)NC(=O)c1cnc[nH]1)C(=O)N[C@@H](C)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₅₂H₈₈N₁₄O₁₃: 1116.67, found [M+3H]³⁺: 377.6.

### Example 257: Compound 257 (SEQ ID NO: 410)

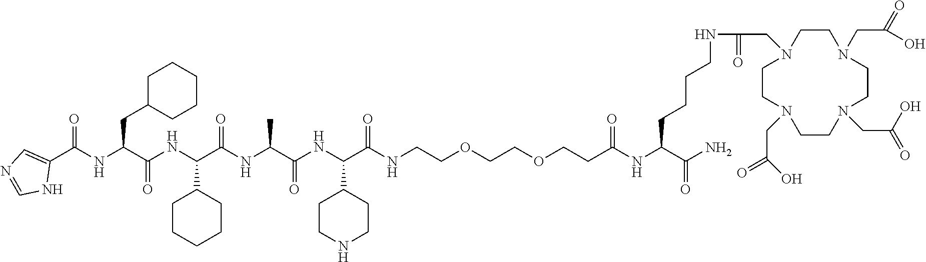 [SMILES: C[C@H](NC(=O)[C@@H](NC(=O)[C@H](CC1CCCCC1)NC(=O)c1cnc[nH]1)C1CCCCC1)C(=O)N[C@H](C(=O)NCCOCCOCCC(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₆₀H₁₀₁N₁₅O₁₆: 1287.76, found [M+3H]³⁺: 430.6.

### Example 258: Compound 258 (SEQ ID NO: 411)

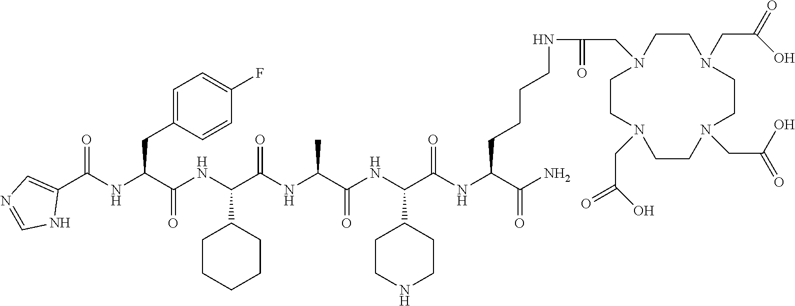 [SMILES: C[C@H](NC(=O)[C@@H](NC(=O)[C@H](Cc1ccc(F)cc1)NC(=O)c1cnc[nH]1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₅₃H₃₁FN₁₄O₁₃: 1140.61, found [M+3H]⁺: 381.6.

### Example 259: Compound 259 (SEQ ID NO: 412)

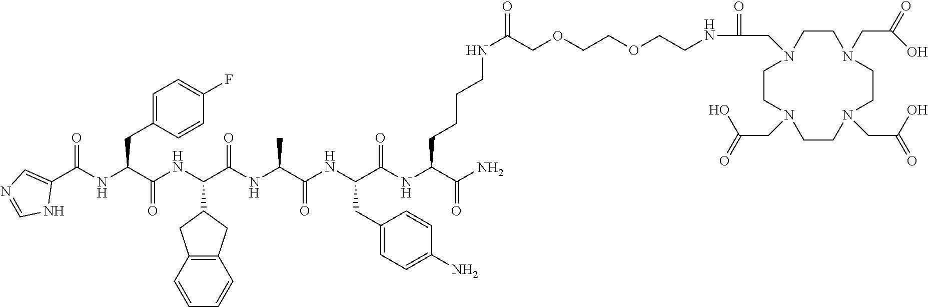 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@@H](Cc1ccc(N)cc1)C(=O)N[C@@H](CCCCNC(=O)COCCOCCNC(=O)CN1CCN(CC(=O)O)CCN(CC(=O)O)CCN(CC(=O)O)CC1)C(N)=O]

MS: Calc'd for C₆₄H₈₈FN₁₅O₁₆: 1341.65, found [M+3H]³⁺: 448.6.

### Example 260: Compound 260 (SEQ ID NO: 413)

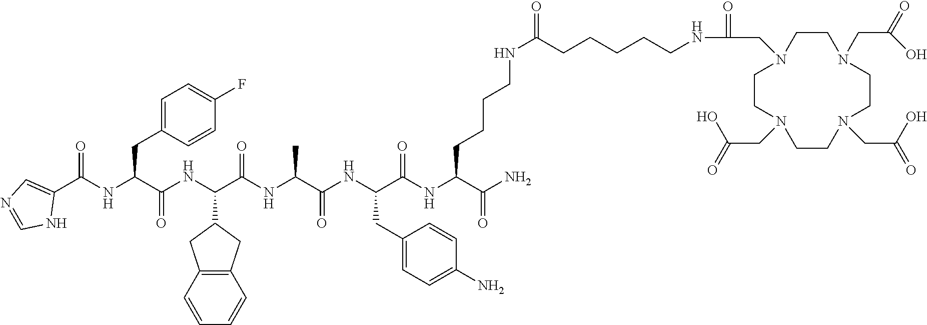 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@@H](Cc1ccc(N)cc1)C(=O)N[C@@H](CCCCNC(=O)CCCCCNC(=O)CN1CCN(CC(=O)O)CCN(CC(=O)O)CCN(CC(=O)O)CC1)C(N)=O]

MS: Calc'd for C₆₄H₈₈FN₁₅O₁₄: 1309.66, found [M+3H]³⁺: 437.9.

### Example 261: Compound 261 (SEQ ID NO: 414)

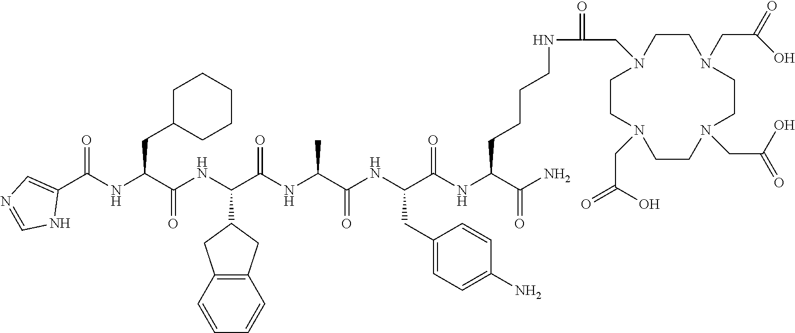 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(N)=O]

MS: Calc'd for C₅₈H₈₄N₁₄O₁₃: 1184.63, found [M+2H]²⁺: 593.75.

### Example 262: Compound 262 (SEQ ID NO: 415)

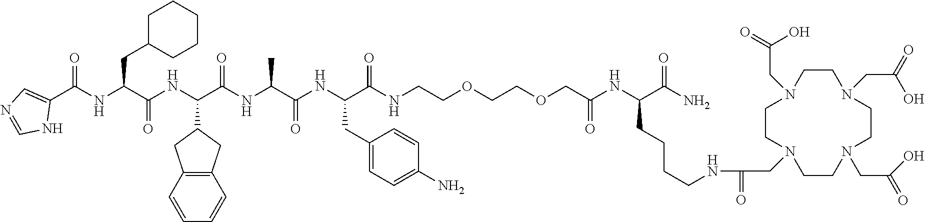 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)NCCOCCOCC(=O)N[C@H](CCCCNC(=O)CN1CCN(CC(=O)O)CCN(CC(=O)O)CCN(CC(=O)O)CC1)C(N)=O]

MS: Calc'd for C₆₄H₉₅N₁₅O₁₆: 1329.71, found [M+2H]²⁺: 666.25.

### Example 263: Compound 263 (SEQ ID NO: 416)

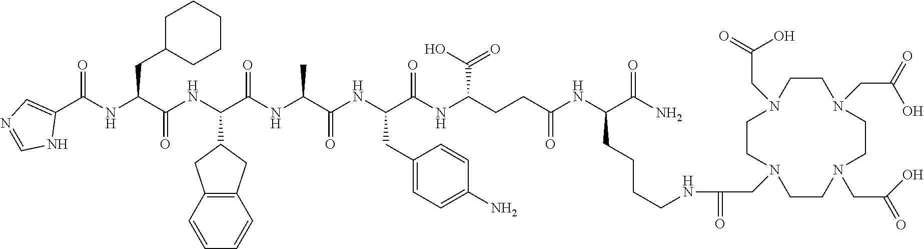 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCC(=O)N[C@H](CCCCNC(=O)CN1CCN(CC(=O)O)CCN(CC(=O)O)CCN(CC(=O)O)CC1)C(N)=O)C(=O)O]

MS: Calc'd for C₆₃H₉₁N₁₅O₁₆: 1313.68, found [M+3H]³⁺: 439.25.

### Example 264: Compound 264 (SEQ ID NO: 417)

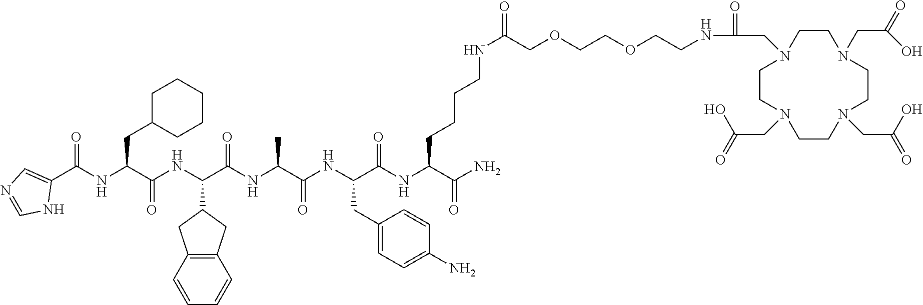 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCCNC(=O)COCCOCCNC(=O)CN1CCN(CC(=O)O)CCN(CC(=O)O)CCN(CC(=O)O)CC1)C(N)=O]

MS: Calc'd for C₆₄H₉₅N₁₅O₁₆: 1329.71, found [M+3H]⁺: 444.6.

### Example 265: Compound 265 (SEQ ID NO: 418)

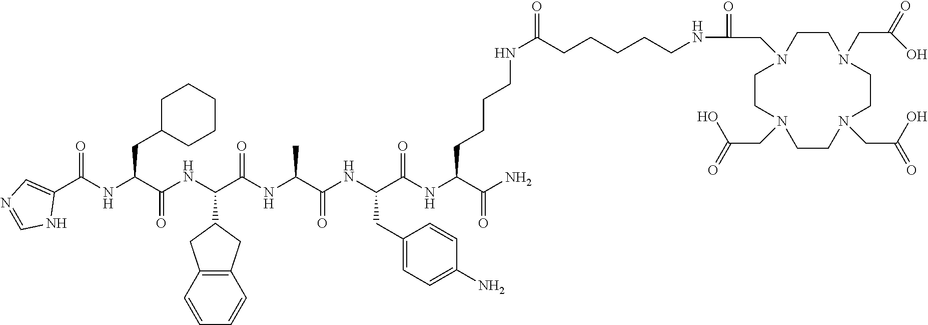 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCCNC(=O)CCCCCNC(=O)CN1CCN(CC(=O)O)CCN(CC(=O)O)CCN(CC(=O)O)CC1)C(N)=O]

MS: Calc'd for C₆₄H₉₅N₁₅O₁₄: 1297.72, found [M+3H]⁺: 433.9.

### Example 266: Compound 266 (SEQ ID NO: 419)

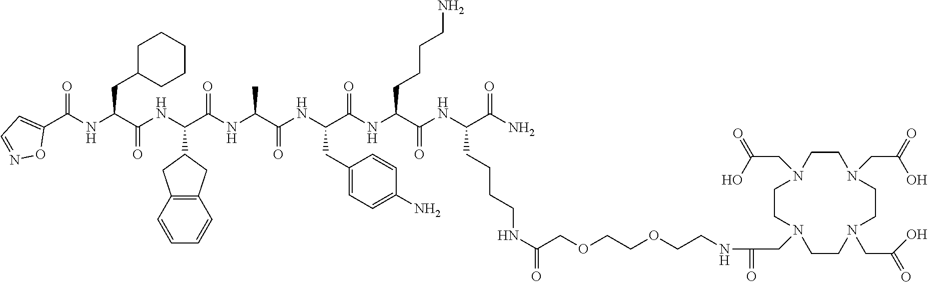 [SMILES: C[C@H](NC(=O)[C@@H](NC(=O)[C@H](CC1CCCCC1)NC(=O)c1ccno1)C1Cc2ccccc2C1)C(=O)N[C@@H](Cc1ccc(N)cc1)C(=O)N[C@@H](CCCCN)C(=O)N[C@@H](CCCCNC(=O)COCCOCCNC(=O)CN1CCN(CC(=O)O)CCN(CC(=O)O)CCN(CC(=O)O)CC1)C(N)=O]

MS: Calc'd for C₇₀H₁₀₆N₁₆O₁₈: 1458.79, found [M+3H]³⁺: 487.6.

### Example 267: Compound 267 (SEQ ID NO: 420)

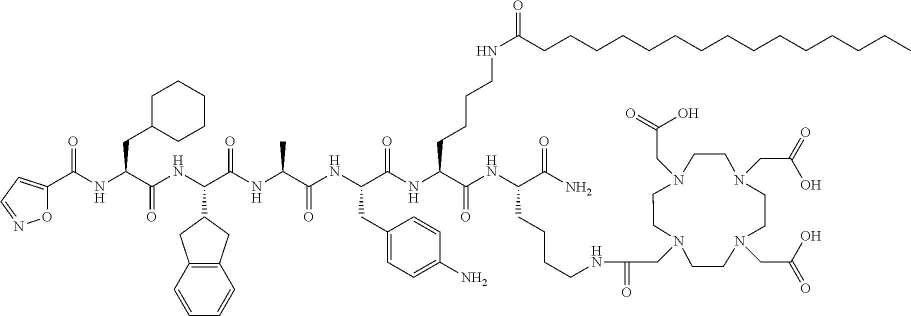 [SMILES: CCCCCCCCCCCCCCCC(=O)NCCCC[C@H](NC(=O)[C@H](Cc1ccc(N)cc1)NC(=O)[C@H](C)NC(=O)[C@@H](NC(=O)[C@H](CC1CCCCC1)NC(=O)c1ccno1)C1Cc2ccccc2C1)C(=O)N[C@@H](CCCCNC(=O)CN1CCN(CC(=O)O)CCN(CC(=O)O)CCN(CC(=O)O)CC1)C(N)=O]

MS: Calc'd for C₈H₁₂₅N₁₅O₁₆: 1551.94, found [M+2H]²⁺: 777.2.

### Example 268: Compound 268 (SEQ ID NO: 421)

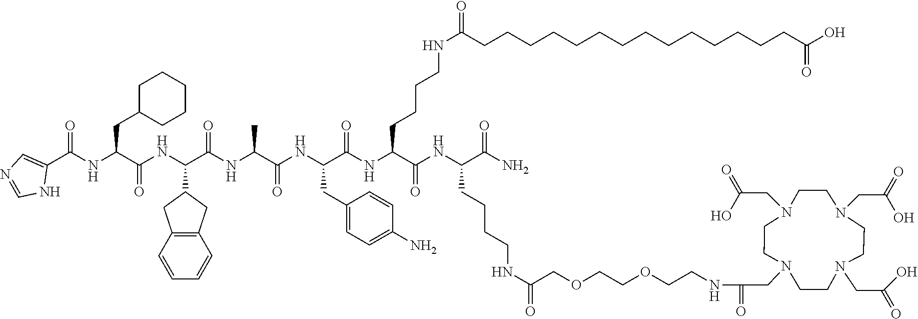 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCCNC(=O)CCCCCCCCCCCCCCC(=O)O)C(=O)N[C@@H](CCCCNC(=O)COCCOCCNC(=O)CN1CCN(CC(=O)O)CCN(CC(=O)O)CCN(CC(=O)O)CC1)C(N)=O]

MS: Calc'd for C₈₆H₁₃₅N₁₇O₂₀: 1726.01, found [M+3H]³⁺: 576.8.

### Example 269: Compound 269 (SEQ ID NO: 422)

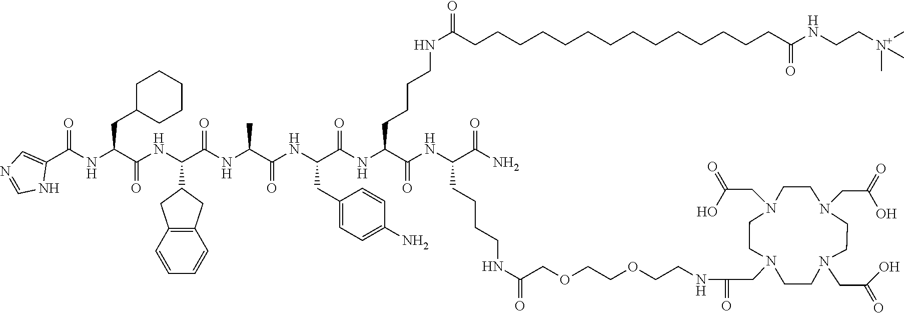 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCCNC(=O)CCCCCCCCCCCCCCC(=O)NCC[N+](C)(C)C)C(=O)N[C@@H](CCCCNC(=O)COCCOCCNC(=O)CN1CCN(CC(=O)O)CCN(CC(=O)O)CCN(CC(=O)O)CC1)C(N)=O]

MS: Calc'd for C₉₁H₁₄SN₁₉O₁₉: 1811.12, found [M+4H]⁴⁺: 453.9.

### Example 270: Compound 270 (SEQ ID NO: 423)

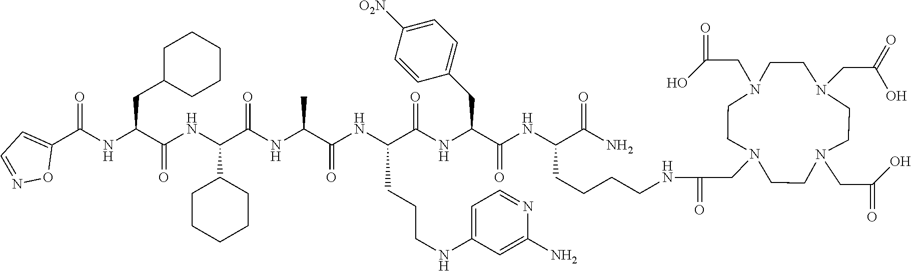 [SMILES: C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N[C@@H](Cc1ccc([N+](=O)[O-])cc1)C(=O)N[C@@H](CCCCNC(=O)CN1CCN(CC(=O)O)CCN(CC(=O)O)CCN(CC(=O)O)CC1)C(N)=O]

MS: Calc'd for C₆₅H₉₇N₁₇O₁₇: 1387.72, found [M+H]⁺: 1388.5.

### Example 271: Compound 271 (SEQ ID NO: 424)

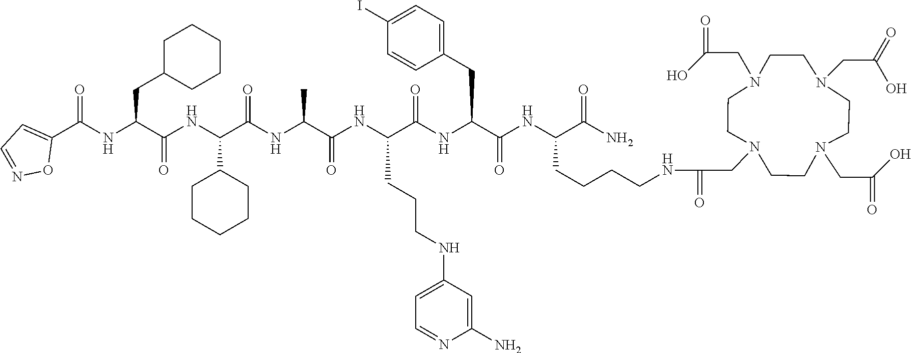 [SMILES: C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N[C@@H](Cc1ccc(I)cc1)C(=O)N[C@@H](CCCCNC(=O)CN1CCN(CC(=O)O)CCN(CC(=O)O)CCN(CC(=O)O)CC1)C(N)=O]

MS: Calc'd for C₆₅H₉₇N₁₆O₁₅: 1468.64, found [M+H]⁺: 1469.6.

### Example 272: Compound 272 (SEQ ID NO: 425)

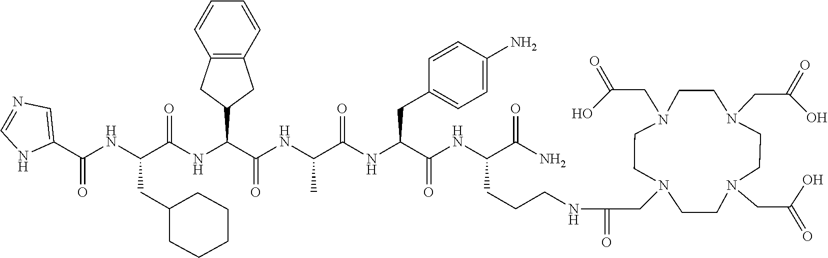 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCNC(=O)CN1CCN(CC(=O)O)CCN(CC(=O)O)CCN(CC(=O)O)CC1)C(N)=O]

MS: Calc'd for C₅₇H₈₂N₁₄O₁₃: 1170.62, found [M+2H]²⁺: 586.65.

### Example 273: Compound 273 (SEQ ID NO: 426)

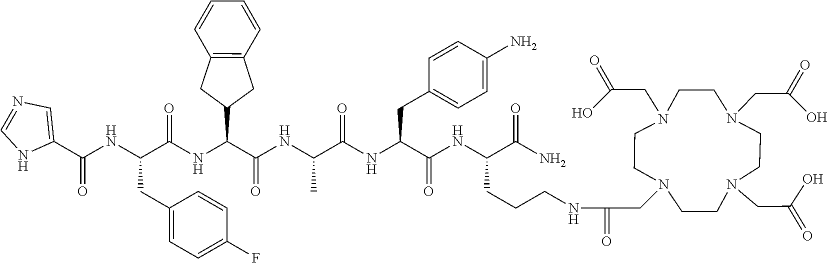 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@@H](Cc1ccc(N)cc1)C(=O)N[C@@H](CCCNC(=O)CN1CCN(CC(=O)O)CCN(CC(=O)O)CCN(CC(=O)O)CC1)C(N)=O]

MS: Calc'd for C₅₇H₇₅FN₁₄O₁₃: 1182.56, found [M+2H]²⁺: 592.7.

### Example 274: Compound 274 (SEQ ID NO: 427)

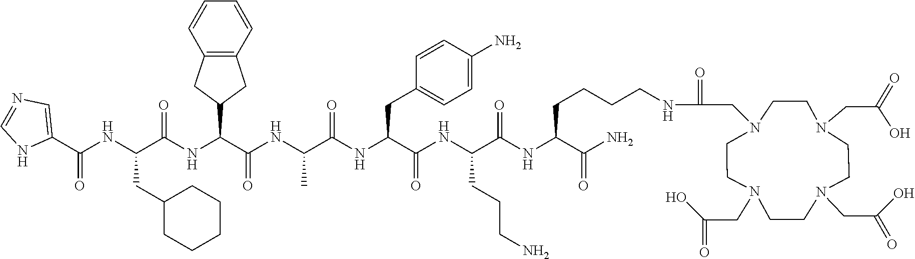 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCN)C(=O)N[C@@H](CCCCNC(=O)CN1CCN(CC(=O)O)CCN(CC(=O)O)CCN(CC(=O)O)CC1)C(N)=O]

MS: Calc'd for C₆₃H₉₄N₁₆O₁₄: 1298.71, found [M+2H]²⁺: 650.7.

### Example 275: Compound 275 (SEQ ID NO: 428)

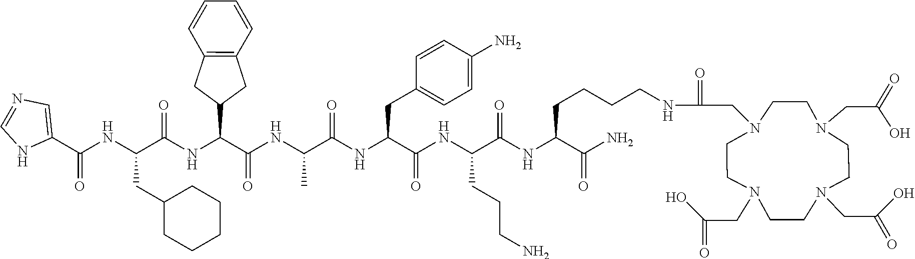 [SMILES: C[C@H](NC(=O)[C@@H](NC(=O)[C@H](CC1CCCCC1)NC(=O)c1cnc[nH]1)C1Cc2ccccc2C1)C(=O)N[C@@H](Cc1ccc(N)cc1)C(=O)N[C@@H](CCCN)C(=O)N[C@@H](CCCCNC(=O)CN1CCN(CC(=O)O)CCN(CC(=O)O)CCN(CC(=O)O)CC1)C(N)=O]

MS: Calc'd for C₆₁H₉₀N₁₆O₁₄: 1270.68, found [M+2H]²⁺: 636.6.

### Example 276: Compound 276 (SEQ ID NO: 429)

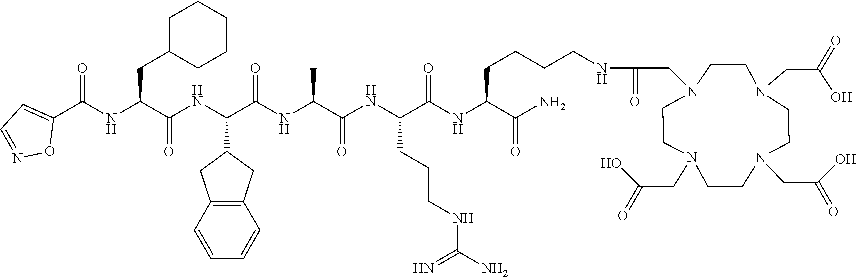 [SMILES: C[C@H](NC(=O)[C@@H](NC(=O)[C@H](CC1CCCCC1)NC(=O)c1ccno1)C1Cc2ccccc2C1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₅H₈₅N₁₅O₁₄: 1179.64, found [M+3H]³⁺: 394.6.

### Example 277: Compound 277 (SEQ ID NO: 430)

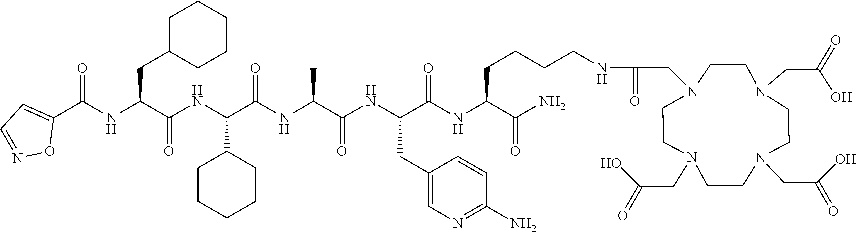 [SMILES: C[C@H](NC(=O)[C@@H](NC(=O)[C@H](CC1CCCCC1)NC(=O)c1ccno1)C1CCCCC1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(N)=O]

### Example 278: Compound 278 (SEQ ID NO: 431)

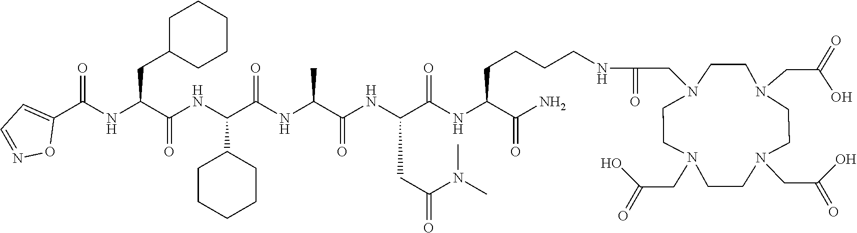 [SMILES: C[C@H](NC(=O)[C@@H](NC(=O)[C@H](CC1CCCCC1)NC(=O)c1ccno1)C1CCCCC1)C(=O)N[C@@H](CC(=O)N(C)C)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₈₅N₁₃O₁₅: 1131.64, found [M+H]⁺: 1132.5.

### Example 279: Compound 279 (SEQ ID NO: 432)

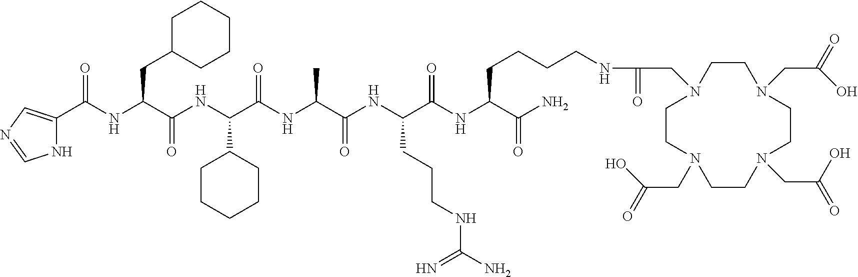 [SMILES: C[C@H](NC(=O)[C@@H](NC(=O)[C@H](CC1CCCCC1)NC(=O)c1cnc[nH]1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₈₈N₁₆O₁₃: 1144.67, found [M+2H]²⁺: 573.8.

### Example 280: Compound 280 (SEQ ID NO: 433)

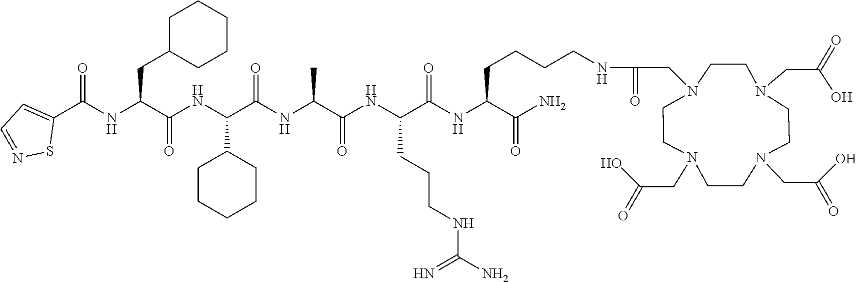 [SMILES: C[C@H](NC(=O)[C@@H](NC(=O)[C@H](CC1CCCCC1)NC(=O)c1ccns1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₈₇N₁₅O₁₃S: 1161.63, found [M+2H]²⁺: 582.3.

### Example 281: Compound 281 (SEQ ID NO: 434)

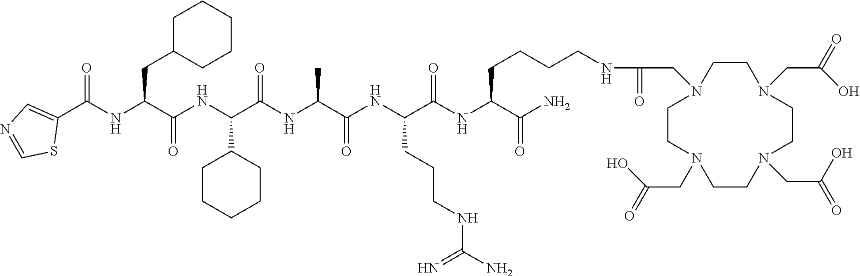 [SMILES: C[C@H](NC(=O)[C@@H](NC(=O)[C@H](CC1CCCCC1)NC(=O)c1cncs1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₈₇N₁₅O₁₃S: 1161.63, found [M+2H]²⁺: 582.3.

### Example 282: (Compound 282) (SEQ ID NO: 435)

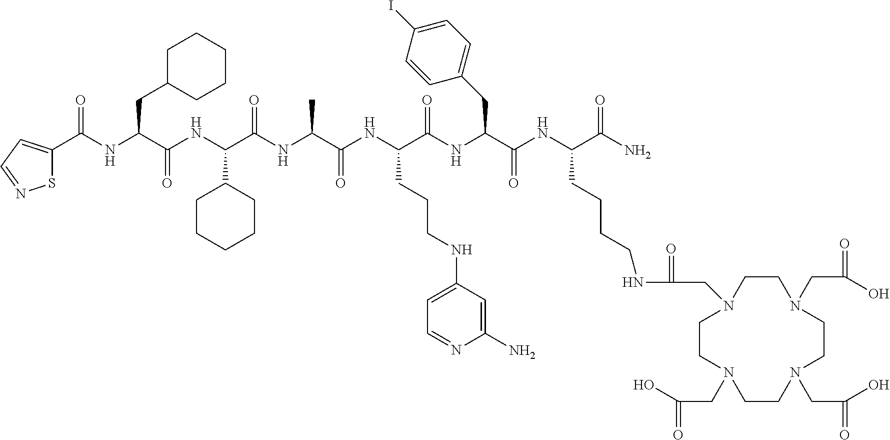 [SMILES: C[C@H](NC(=O)[C@@H](NC(=O)[C@H](CC1CCCCC1)NC(=O)c1ccns1)C1CCCCC1)C(=O)N[C@@H](CCCNc1ccnc(N)c1)C(=O)N[C@@H](Cc1ccc(I)cc1)C(=O)N[C@@H](CCCCNC(=O)CN1CCN(CC(=O)O)CCN(CC(=O)O)CCN(CC(=O)O)CC1)C(N)=O]

MS: Calc'd for C₆₅H₉₇IN₁₆O₁₄S: 1484.61, found [M+H]⁺: 1485.6.

### Example 283: (Compound 283) (SEQ ID NO: 436)

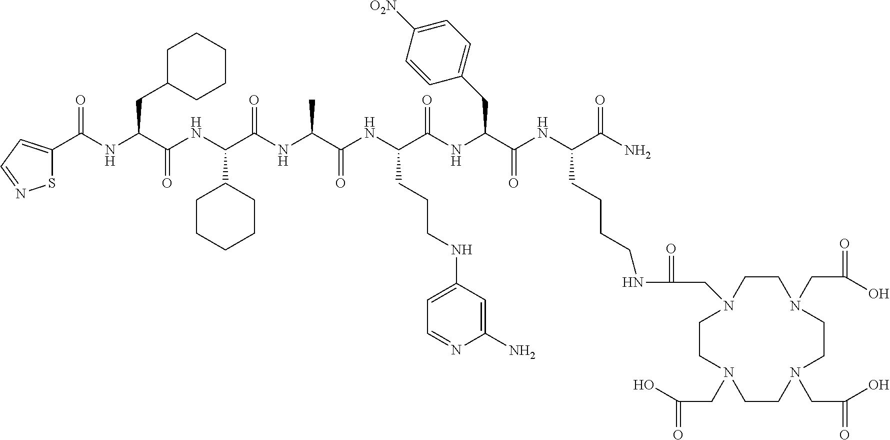 [SMILES: C[C@H](NC(=O)[C@@H](NC(=O)[C@H](CC1CCCCC1)NC(=O)c1ccns1)C1CCCCC1)C(=O)N[C@@H](CCCNc1ccnc(N)c1)C(=O)N[C@@H](Cc1ccc([N+](=O)[O-])cc1)C(=O)N[C@@H](CCCCNC(=O)CN1CCN(CC(=O)O)CCN(CC(=O)O)CCN(CC(=O)O)CC1)C(N)=O]

MS: Calc'd for C₆₅H₉₇N₁₇O₁₆S: 1403.70, found [M+H]⁺: 1404.5.

### Example 284: (Compound 284) (SEQ ID NO: 437)

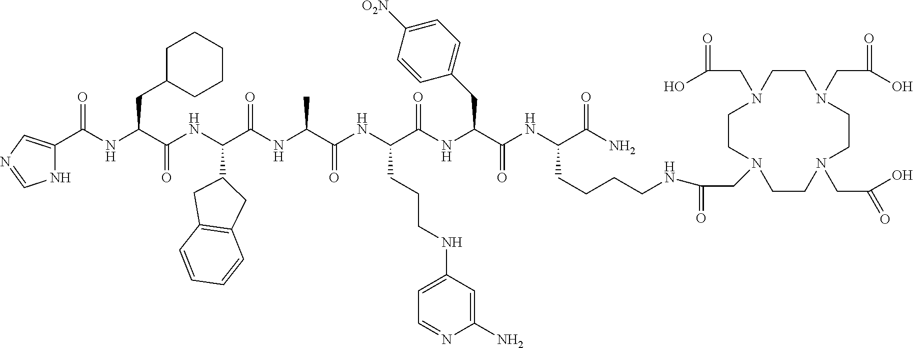 [SMILES: C[C@H](NC(=O)[C@@H](NC(=O)[C@H](CC1CCCCC1)NC(=O)c1cnc[nH]1)C1Cc2ccccc2C1)C(=O)N[C@@H](CCCNc1ccnc(N)c1)C(=O)N[C@@H](Cc1ccc([N+](=O)[O-])cc1)C(=O)N[C@@H](CCCCNC(=O)CN1CCN(CC(=O)O)CCN(CC(=O)O)CCN(CC(=O)O)CC1)C(N)=O]

MS: Calc'd for C₆₅H₉₆N₁₈O₁₆: 1420.73, found [M+H]⁺: 1421.6.

### Example 285: (Compound 285) (SEQ ID NO: 438)

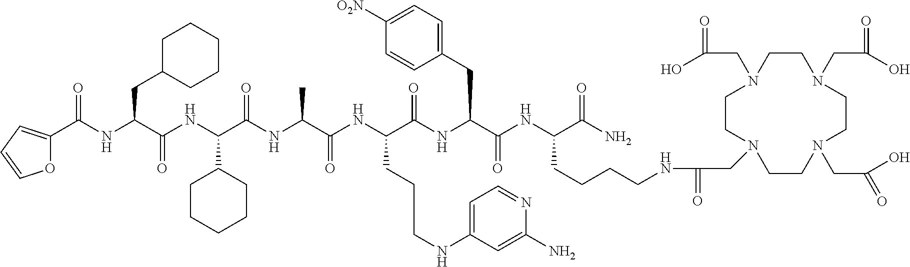 [SMILES: C[C@H](NC(=O)[C@@H](NC(=O)[C@H](CC1CCCCC1)NC(=O)c1ccco1)C1CCCCC1)C(=O)N[C@@H](CCCNc1ccnc(N)c1)C(=O)N[C@@H](Cc1ccc([N+](=O)[O-])cc1)C(=O)N[C@@H](CCCCNC(=O)CN1CCN(CC(=O)O)CCN(CC(=O)O)CCN(CC(=O)O)CC1)C(N)=O]

MS: Calc'd for C₆₆H₉₈N₁₆O₁₇: 1386.73, found [M+H]⁺: 1387.5.

### Example 286: (Compound 286) (SEQ ID NO: 439)

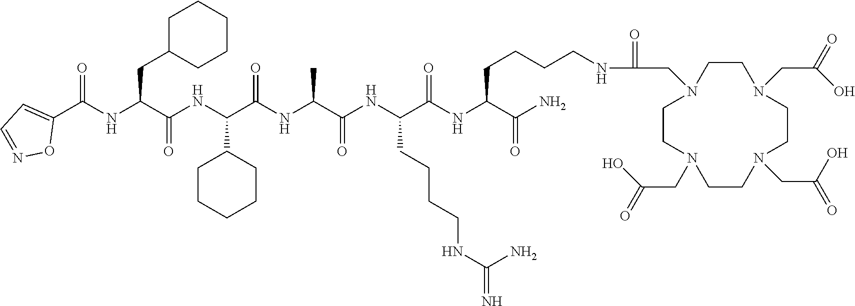 [SMILES: C[C@H](NC(=O)[C@@H](NC(=O)[C@H](CC1CCCCC1)NC(=O)c1ccno1)C1CCCCC1)C(=O)N[C@@H](CCCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₃H₃₉N₁₅O₁₄: 1159.67, found [M+H]⁺: 1160.6.

### Example 287: (Compound 287) (SEQ ID NO: 440)

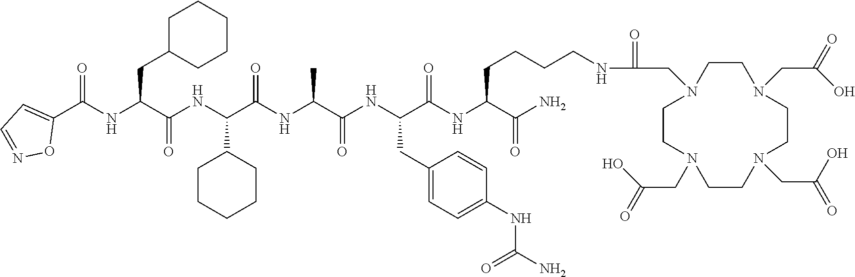 [SMILES: C[C@H](NC(=O)[C@@H](NC(=O)[C@H](CC1CCCCC1)NC(=O)c1ccno1)C1CCCCC1)C(=O)N[C@@H](Cc1ccc(NC(N)=O)cc1)C(=O)N[C@@H](CCCCNC(=O)CN1CCN(CC(=O)O)CCN(CC(=O)O)CCN(CC(=O)O)CC1)C(N)=O]

MS: Calc'd for C₅₆H₈₆N₁₄O₁₅: 1194.64, found [M+H]⁺: 1195.4.

### Example 288: Compound 288 (SEQ ID NO: 441)

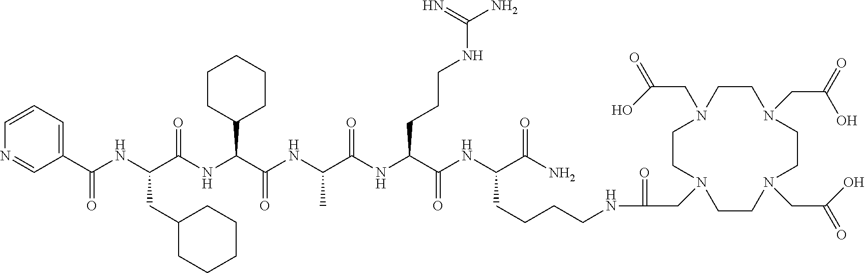 [SMILES: C[C@H](NC(=O)[C@@H](NC(=O)[C@H](CC1CCCCC1)NC(=O)c1cccnc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

### Example 289: (Compound 289) (SEQ ID NO: 442)

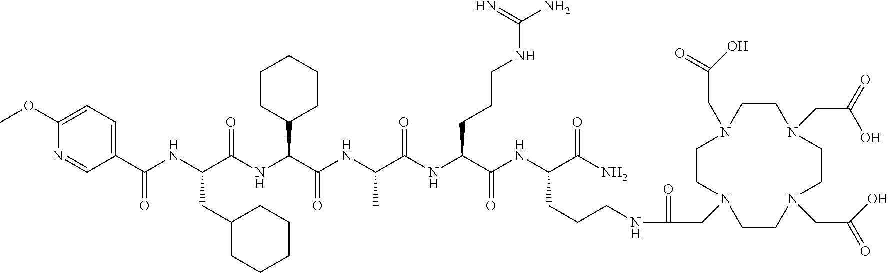 [SMILES: COc1ccc(C(=O)N[C@@H](CC2CCCCC2)C(=O)N[C@H](C(=O)N[C@@H](C)C(=O)N[C@@H](CCCNC(=N)N)C(=O)N[C@@H](CCCNC(=O)CN2CCN(CC(=O)O)CCN(CC(=O)O)CCN(CC(=O)O)CC2)C(N)=O)C2CCCCC2)cn1]

MS: Calc'd for C₅₄H₈₉N₁₅O₁₄: 1171.67, found [M+H]⁺: 1172.9.

### Example 290: (Compound 290) (SEQ ID NO: 443)

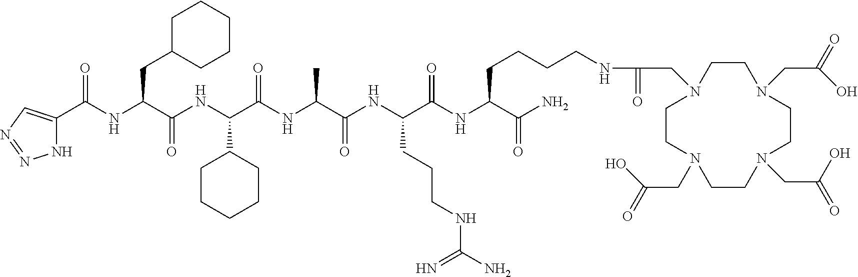 [SMILES: C[C@H](NC(=O)[C@@H](NC(=O)[C@H](CC1CCCCC1)NC(=O)c1cnn[nH]1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₁H₈₇N₁₇O₁₃: 1145.67, found [M+2H]²⁺: 574.1.

### Example 291: (Compound 291) (SEQ ID NO: 444)

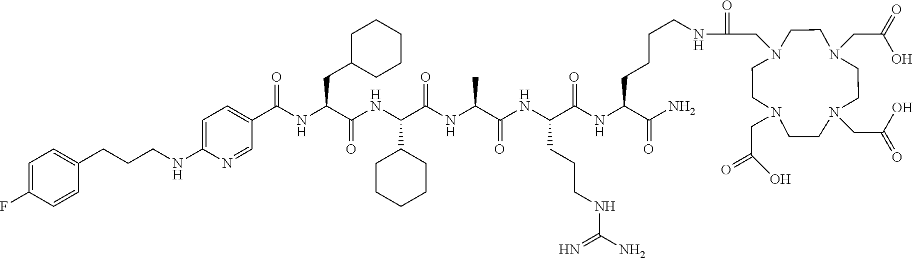 [SMILES: C[C@H](NC(=O)[C@@H](NC(=O)[C@H](CC1CCCCC1)NC(=O)c1ccc(NCCCc2ccc(F)cc2)nc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₆₃H₉₉FN₁₆O₁₃: 1306.76, found [M+H]⁺: 1307.6.

### Example 292: (Compound 292) (SEQ ID NO: 445)

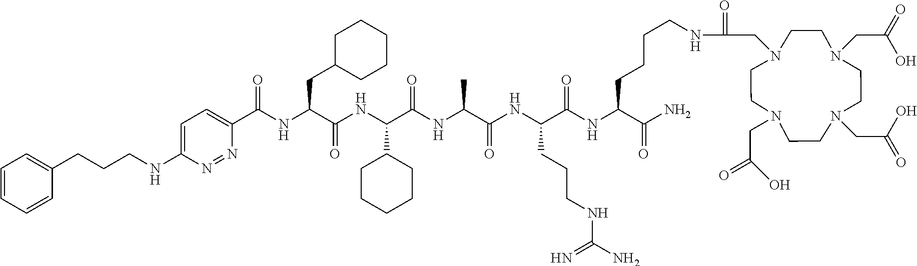 [SMILES: C[C@H](NC(=O)[C@@H](NC(=O)[C@H](CC1CCCCC1)NC(=O)c1ccc(NCCCc2ccccc2)nn1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₆₂H₉,N₁₇O₁₃: 1289.76, found [M+H]⁺: 1290.6.

### Example 293: (Compound 293) (SEQ ID NO: 446)

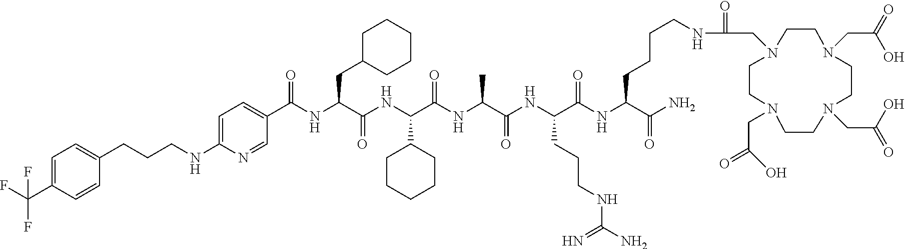 [SMILES: C[C@H](NC(=O)[C@@H](NC(=O)[C@H](CC1CCCCC1)NC(=O)c1ccc(NCCCc2ccc(C(F)(F)F)cc2)nc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₆₄H₉₉FN₁₆O₁₃: 1356.75, found [M+H]⁺: 1357.6.

### Example 294: (Compound 294) (SEQ ID NO: 447)

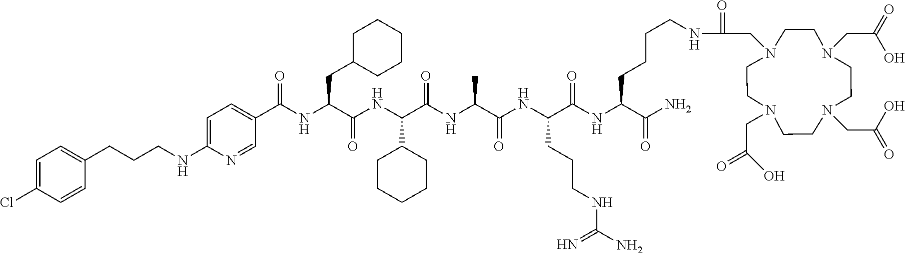 [SMILES: C[C@H](NC(=O)[C@@H](NC(=O)[C@H](CC1CCCCC1)NC(=O)c1ccc(NCCCc2ccc(Cl)cc2)nc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₆₃H₉₉ClN₁₆O₁₃: 1322.73, found [M+H]⁺: 1323.7.

### Example 295: (Compound 295) (SEQ ID NO: 448)

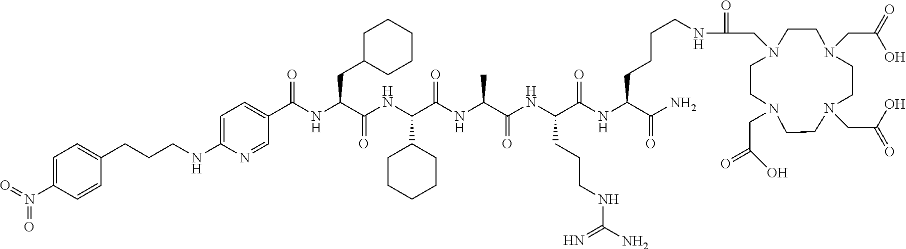 [SMILES: C[C@H](NC(=O)[C@@H](NC(=O)[C@H](CC1CCCCC1)NC(=O)c1ccc(NCCCc2ccc([N+](=O)[O-])cc2)nc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₆₃H₉₉N₁₇O₁₅: 1333.75, found [M+H]⁺: 1334.7.

### Example 296: (Compound 296) (SEQ ID NO: 449)

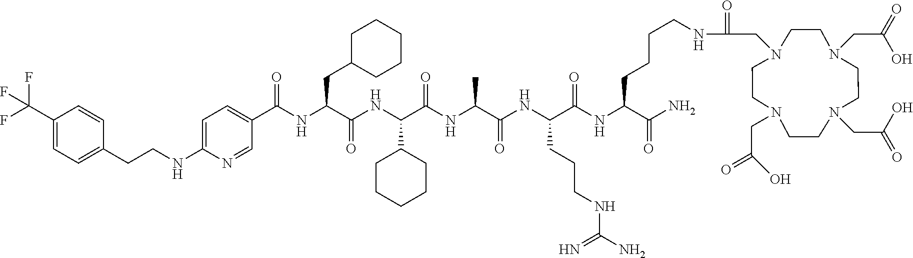 [SMILES: C[C@H](NC(=O)[C@@H](NC(=O)[C@H](CC1CCCCC1)NC(=O)c1ccc(NCCc2ccc(C(F)(F)F)cc2)nc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₆₃H₉₇F₃N₁₆O₁₃: 1342.73, found [M+H]⁺: 1343.6.

### Example 297: (Compound 297) (SEQ ID NO: 450)

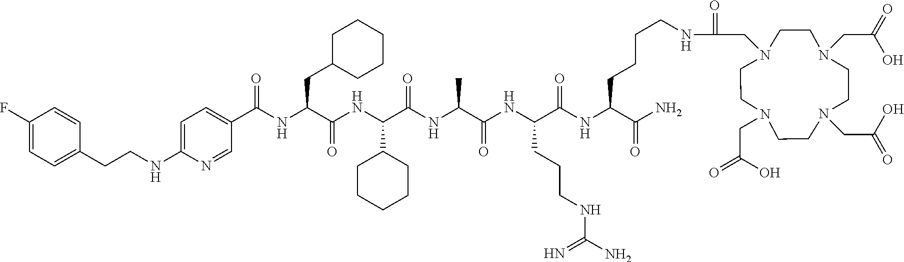 [SMILES: C[C@H](NC(=O)[C@@H](NC(=O)[C@H](CC1CCCCC1)NC(=O)c1ccc(NCCc2ccc(F)cc2)nc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₆₂H₉₇FN₁₆O₁₃: 1292.74, found [M+H]⁺: 1294.1.

### Example 298: (Compound 298) (SEQ ID NO: 451)

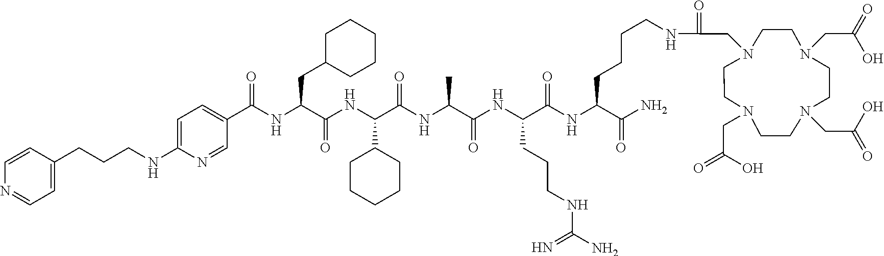 [SMILES: C[C@H](NC(=O)[C@@H](NC(=O)[C@H](CC1CCCCC1)NC(=O)c1ccc(NCCCc2ccncc2)nc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₆₂H₉₉N₁₇O₁₃: 1289.76, found [M+H]⁺: 1290.9.

### Example 299: (Compound 299) (SEQ ID NO: 452)

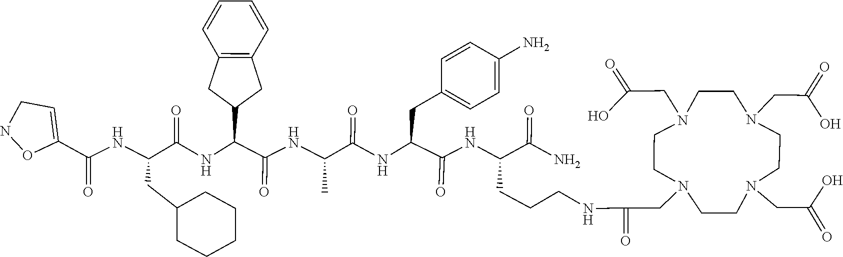

MS: Calc'd for C₅₇H₈₁N₁₃O₁₄: 1171.60, found [M+2H]²⁺: 587.2.

### Example 300: (Compound 300) (SEQ ID NO: 453)

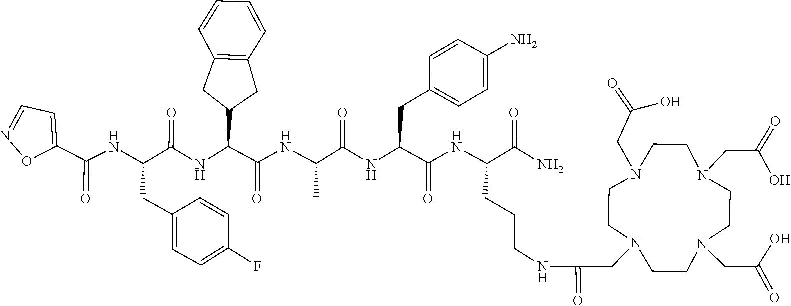 [SMILES: C[C@H](NC(=O)[C@@H](NC(=O)[C@H](Cc1ccc(F)cc1)NC(=O)c1ccno1)C1Cc2ccccc2C1)C(=O)N[C@@H](Cc1ccc(N)cc1)C(=O)N[C@@H](CCCNC(=O)CN1CCN(CC(=O)O)CCN(CC(=O)O)CCN(CC(=O)O)CC1)C(N)=O]

MS: Calc'd for C₅₇H₇₄FN₁₃O₁₄: 1183.55, found [M+2H]²⁺: 593.15.

### Example 301: (Compound 301) (SEQ ID NO: 454)

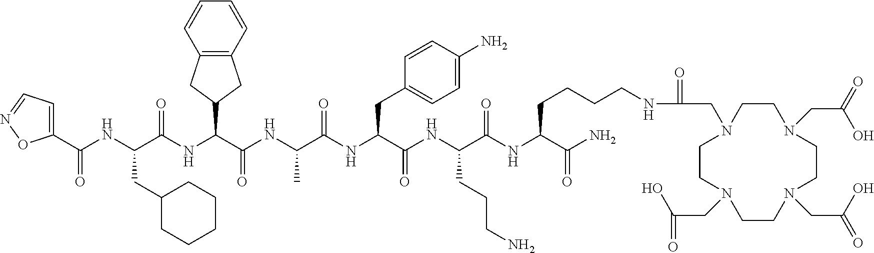 [SMILES: C[C@H](NC(=O)[C@@H](NC(=O)[C@H](CC1CCCCC1)NC(=O)c1ccno1)C1Cc2ccccc2C1)C(=O)N[C@@H](Cc1ccc(N)cc1)C(=O)N[C@@H](CCCN)C(=O)N[C@@H](CCCCNC(=O)CN1CCN(CC(=O)O)CCN(CC(=O)O)CCN(CC(=O)O)CC1)C(N)=O]

MS: Calc'd for C₆₃H₉₃N₁₅O₁₅: 1299.70, found [M+2H]²⁺: 651.05.

### Example 302: (Compound 302) (SEQ ID NO: 455)

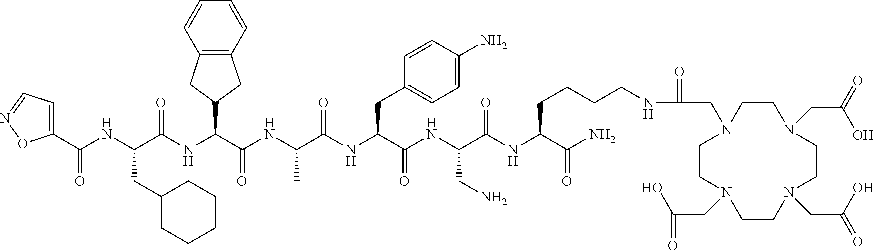 [SMILES: C[C@H](NC(=O)[C@@H](NC(=O)[C@H](CC1CCCCC1)NC(=O)c1ccno1)C1Cc2ccccc2C1)C(=O)N[C@@H](Cc1ccc(N)cc1)C(=O)N[C@@H](CN)C(=O)N[C@@H](CCCCNC(=O)CN1CCN(CC(=O)O)CCN(CC(=O)O)CCN(CC(=O)O)CC1)C(N)=O]

MS: Calc'd for C₆₁H₈₉N₁₅O₁₅: 1271.67, found [M+2H]²⁺: 637.0.

### Example 303: (Compound 303) (SEQ ID NO: 456)

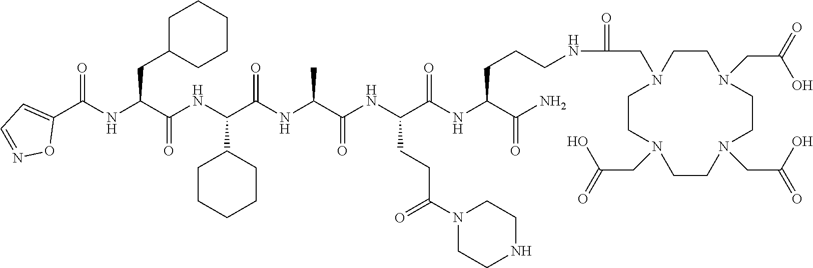 [SMILES: C[C@H](NC(=O)[C@@H](NC(=O)[C@H](CC1CCCCC1)NC(=O)c1ccno1)C1CCCCC1)C(=O)N[C@@H](CCC(=O)N1CCNCC1)C(=O)N[C@@H](CCCNC(=O)CN1CCN(CC(=O)O)CCN(CC(=O)O)CCN(CC(=O)O)CC1)C(N)=O]

### Synthesis of (S)-2-((((9H-fluoren-9-yl) methoxy) carbonyl)amino)-5-(4-((benzyloxy) carbonyl) piperazin-1-yl)-5-oxopentanoic acid

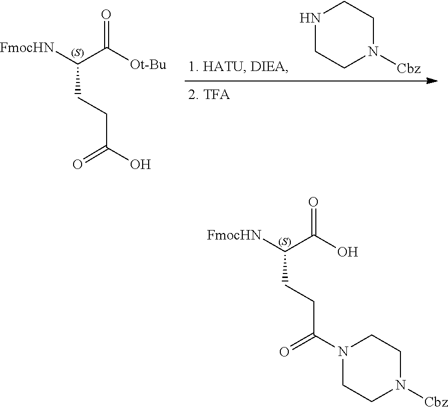

Step 1: A mixture of(S)-4-((((9H-fluoren-9-yl) methoxy) carbonyl)amino)-5-(tert-butoxy)-5-oxopentanoic acid (7.9 g, 1 Eq, 19 mmol), HATU (6.0 g, 1 Eq, 16 mmol), diisopropylethylamine (5 g, 7 mL, 3 Eq, 0.04 mol), benzyl piperazine-1-carboxylate (3 g, 1 Eq, 0.01 mol) and DMF (30 mL) was stirred at 25° C. for 2 hours. The mixture was directly purified by Prep-HPLC to afford benzyl(S)-4-(4-((((9H-fluoren-9-yl) methoxy) carbonyl)amino)-5-(tert-butoxy)-5-oxopentanoyl) piperazine-1-carboxylate (4.6 g, 7.3 mmol, 50%) as a white solid.

Step 2: A mixture of benzyl(S)-4-(4-((((9H-fluoren-9-yl) methoxy) carbonyl)amino)-5-(tert-butoxy)-5-oxopentanoyl) piperazine-1-carboxylate (4.6 g, 1 Eq, 7.3 mmol), DCM (15 mL) and TFA (15 mL) was stirred at 60° C. for 1 hour. The mixture was concentrated under reduced pressure then directly purified by Prep-HPLC to afford(S)-2-((((9H-fluoren-9-yl) methoxy) carbonyl)amino)-5-(4-((benzyloxy) carbonyl) piperazin-1-yl)-5-oxopentanoic acid (3.2 g, 5.6 mmol, 76%) as a white solid. MS: Calc'd for C₃₂H₃₃N₃O₇: 571.23, found [M+H]⁺: 572.2.

Compound 303 was synthesized according to the generic procedures described above to provide the product. MS: Calc'd for C₅₄H₈₈N₁₄O₁₅: 1172.66, found [M+H]⁺: 1173.6.

### Example 304: Compound 304

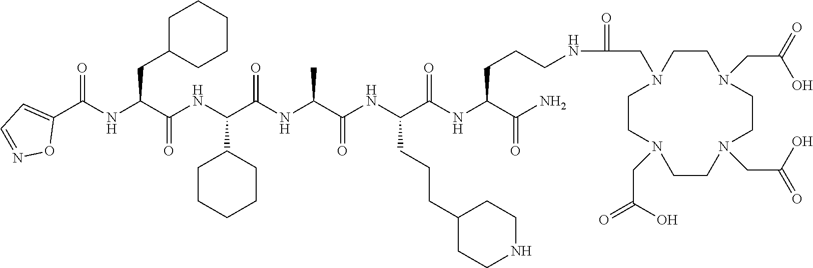 [SMILES: C[C@H](NC(=O)[C@@H](NC(=O)[C@H](CC1CCCCC1)NC(=O)c1ccno1)C1CCCCC1)C(=O)N[C@@H](CCCC1CCNCC1)C(=O)N[C@@H](CCCNC(=O)CN1CCN(CC(=O)O)CCN(CC(=O)O)CCN(CC(=O)O)CC1)C(N)=O]

### Synthesis of(S)-2-((((9H-fluoren-9-yl) methoxy) carbonyl)amino)-5-(1-((benzyloxy) carbonyl) piperidin-4-yl) pentanoic acid

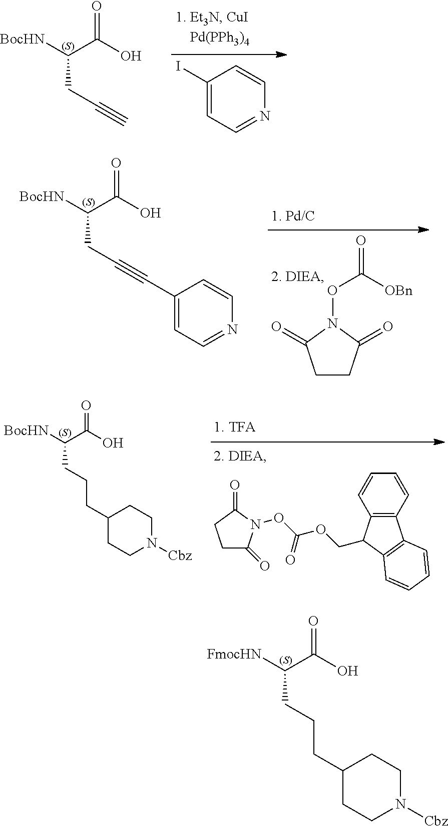

Step 1: A mixture of(S)-2-((tert-butoxycarbonyl)amino) pent-4-ynoic acid (2.0 g, 1 Eq, 9.4 mmol), Pd(PPh₃) ₄ (0.54 g, 0.050 Eq, 0.47 mmol), 4-iodopyridine (2.1 g, 1.1 Eq, 10 mmol), CuI (0.18 g, 0.10 Eq, 0.95 mmol), TEA (2.8 g, 3.9 mL, 3.0 Eq, 28 mmol) and DMF (40 mL) was stirred at 40° C. for 1 hour under a N₂ environment. The mixture was directly purified by MPLC to afford (S)-2-((tert-butoxycarbonyl)amino)-5-(pyridin-4-yl) pent-4-ynoic acid (1.05 g, 3.62 mmol, 39%) as a yellow oil. [M+H]⁺=291.2.

Step 2: Into a 100-mL round-bottom flask purged and maintained with an inert atmosphere of nitrogen, was placed(S)-2-((tert-butoxycarbonyl)amino)-5-(pyridin-4-yl) pent-4-ynoic acid (1.0 g, 1 Eq, 3.4 mmol) and AcOH (20 mL), to which was carefully added 10% Pd/C (0.92 g, 2.5 Eq, 8.6 mmol). The flask was evacuated and flushed with hydrogen three times, then the mixture was stirred at 60° C. for 3 hours. The reaction mixture was filtered through a pad of celite, then the filtrate was concentrated and dried, to provide crude(S)-2-((tert-butoxycarbonyl)amino)-5-(piperidin-4-yl) pentanoic acid (0.95 g, 1.6 mmol, 46%, 50% Purity) as a colorless oil, which was used in the next step without further purification.

Step 3: Into a 8-mL vial, was placed a mixture of(S)-2-((tert-butoxycarbonyl)amino)-5-(piperidin-4-yl) pentanoic acid (950 mg, 50% Wt, 1 Eq, 1.58 mmol), DIEA (613 mg, 826 μL, 3.00 Eq, 4.74 mmol), benzyl (2,5-dioxopyrrolidin-1-yl) carbonate (394 mg, 1.00 Eq, 1.58 mmol) and DMF (20 mL). The reaction mixture was stirred at 25° C. for 1 hour. The mixture was directly purified by MPLC to afford(S)-5-(1-((benzyloxy) carbonyl) piperidin-4-yl)-2-((tert-butoxycarbonyl)amino) pentanoic acid (270 mg, 621 μmol, 39.3%) as a white solid. [M+H-Boc]⁺=335.2.

Step 4: To a mixture of(S)-5-(1-((benzyloxy) carbonyl) piperidin-4-yl)-2-((tert-butoxycarbonyl)amino) pentanoic acid (270 mg, 1 Eq, 621 μmol) and DCM (3 mL) was added TFA (1 mL). The reaction mixture was stirred at 25° C. for 1 hour. The mixture was concentrated under reduced pressure to afford(S)-2-amino-5-(1-((benzyloxy) carbonyl) piperidin-4-yl) pentanoic acid (200 mg, 598 μmol, 96.2%) as a yellow oil, which was used directly in the next step without further purification. [M+H]⁺=335.1.

Step 5: A mixture of(S)-2-amino-5-(1-((benzyloxy) carbonyl) piperidin-4-yl) pentanoic acid (200 mg, 1 Eq, 598 μmol), DIEA (230 mg, 310 μL, 2.98 Eq, 1.78 mmol), Fmoc-OSu (303 mg, 1.50 Eq, 898 μmol) and DMF (20 mL) was stirred at 25° C. for 1 hour. The mixture was directly purified by MPLC to afford(S)-5-(1-((benzyloxy) carbonyl) piperidin-4-yl)-2-((tert-butoxycarbonyl)amino) pentanoic acid (270 mg, 621 μmol, 39.3%) as a white solid. MS: Calc'd for C₃₃H₃₆N₂O₆: 556.26, found [M+H]⁺: 557.4.

Compound 304 was synthesized according to the generic procedures described above to provide the product. MS: Calc'd for C₅₅H₉₁N₁₃O₁₄: 1157.68, found [M+H]⁺: 1158.5.

### Example 305: Compound 305

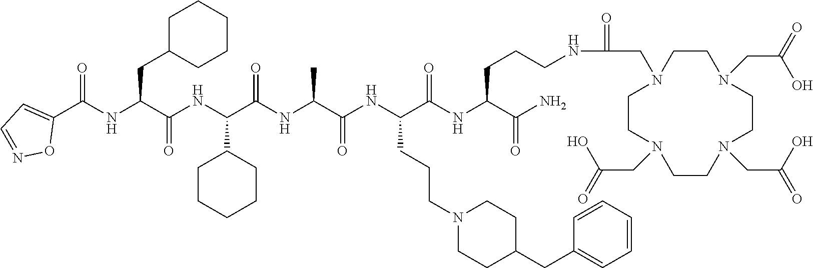 [SMILES: C[C@H](NC(=O)[C@@H](NC(=O)[C@H](CC1CCCCC1)NC(=O)c1ccno1)C1CCCCC1)C(=O)N[C@@H](CCCN1CCC(Cc2ccccc2)CC1)C(=O)N[C@@H](CCCNC(=O)CN1CCN(CC(=O)O)CCN(CC(=O)O)CCN(CC(=O)O)CC1)C(N)=O]

### Synthesis of(S)-2-((((9H-fluoren-9-yl) methoxy) carbonyl)amino)-5-(4-benzylpiperidin-1-yl) pentanoic acid

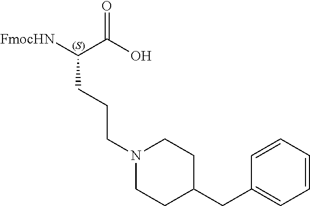

(S)-2-((((9H-fluoren-9-yl) methoxy) carbonyl)amino)-5-(4-benzylpiperidin-1-yl) pentanoic acid was synthesized from(S)-4-((((9H-fluoren-9-yl) methoxy) carbonyl)amino)-5-(tert-butoxy)-5-oxopentanoic acid and 1-benzylpiperidine in a manner similar to that described in Example 304, to provide the product (600 mg, 1.17 mmol, 22%) as a white solid. MS: Calc'd for C₃₂H₃₆N₂O₄: 512.27, found [M+H]⁺: 513.3.

Compound 305 was synthesized according to the generic procedures described above to provide the product. MS: Calc'd for C₆₂H₉₇N₁₃O₁₄: 1247.73, found [M+H]⁺: 1248.6.

### Example 306: Compound 306

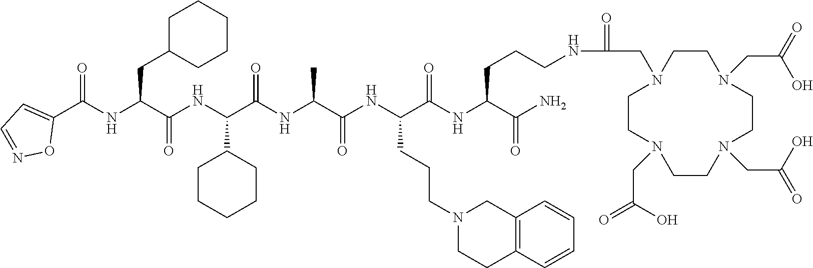 [SMILES: C[C@H](NC(=O)[C@@H](NC(=O)[C@H](CC1CCCCC1)NC(=O)c1ccno1)C1CCCCC1)C(=O)N[C@@H](CCCN1CCc2ccccc2C1)C(=O)N[C@@H](CCCNC(=O)CN1CCN(CC(=O)O)CCN(CC(=O)O)CCN(CC(=O)O)CC1)C(N)=O]

### Synthesis of(S)-2-((((9H-fluoren-9-yl) methoxy) carbonyl)amino)-5-(3,4-dihydroisoquinolin-2 (1H)-yl) pentanoic acid

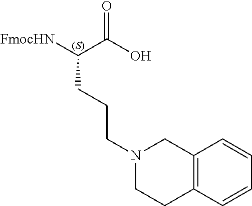

(S)-2-((((9H-fluoren-9-yl) methoxy) carbonyl)amino)-5-(3,4-dihydroisoquinolin-2 (1H)-yl) pentanoic acid was synthesized from(S)-4-((((9H-fluoren-9-yl) methoxy) carbonyl)amino)-5-(tert-butoxy)-5-oxopentanoic acid and 1,2,3,4-tetrahydroisoquinoline in a manner similar to that described in Example 304, to provide the product (3.0 g, 5.7 mmol, 70%) as a white solid. MS: Calc'd for C₂₉H₃₀N₂O₄: 470.22, found [M+H]⁺: 471.2.

Compound 306 was synthesized according to the generic procedures described above to provide the product. MS: Calc'd for C₅₉H₉₁N₁₃O₁₄: 1205.68, found [M+H]⁺: 1206.5.

### Example 307: (Compound 307) (SEQ ID NO: 460)

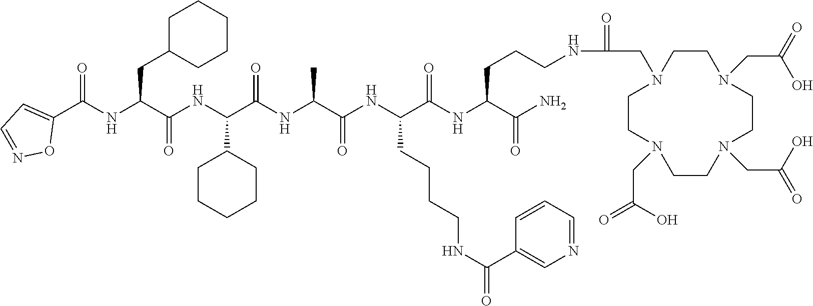 [SMILES: C[C@H](NC(=O)[C@@H](NC(=O)[C@H](CC1CCCCC1)NC(=O)c1ccno1)C1CCCCC1)C(=O)N[C@@H](CCCCNC(=O)c1cccnc1)C(=O)N[C@@H](CCCNC(=O)CN1CCN(CC(=O)O)CCN(CC(=O)O)CCN(CC(=O)O)CC1)C(N)=O]

### Synthesis of N2-(((9H-fluoren-9-yl) methoxy) carbonyl)-N6-nicotinoyl-L-lysine

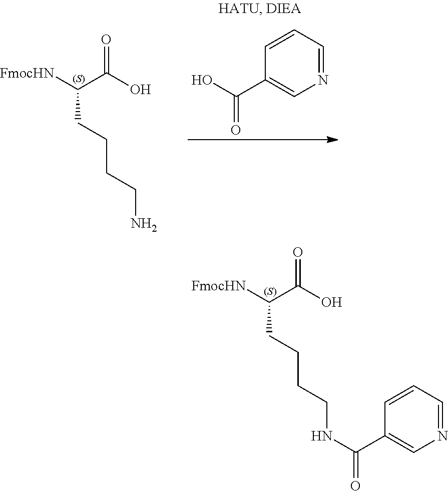

A mixture of (((9H-fluoren-9-yl) methoxy) carbonyl)-L-lysine (3.0 g, 1 Eq, 8.1 mmol), HATU (3.1 g, 1.0 Eq, 8.2 mmol), diisopropylethylamine (3.2 g, 4.3 mL, 3.0 Eq, 25 mmol), nicotinic acid (1.0 g, 1.0 Eq, 8.1 mmol) and DMF (30 mL) was stirred at 25° C. for 2 hours. The crude mixture was directly purified by Prep-HPLC to provide N2-(((9H-fluoren-9-yl) methoxy) carbonyl)-N6-nicotinoyl-L-lysine (1.6 g, 3.4 mmol, 41%) as a yellow oil. MS: Calc'd for C₂₉H₂₇N₃O₅: 473.20, found [M+H]⁺: 474.2.

Compound 307 was synthesized according to the generic procedures described above to provide the product. MS: Calc'd for C₅₇H₈₈N₁₄O₁₅: 1208.66, found [M+H]⁺: 1209.5.

### Example 308: (Compound 308) (SEQ ID NO: 461)

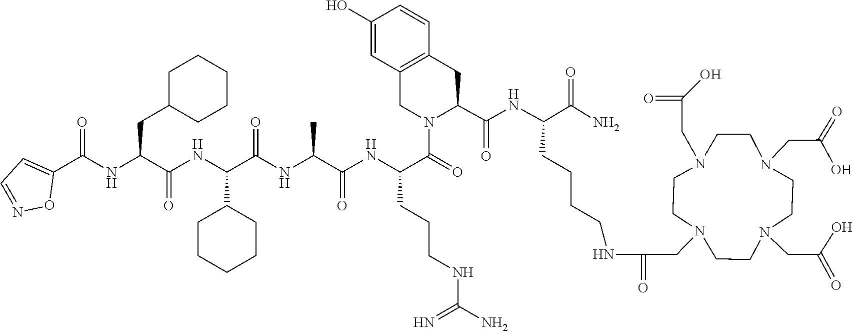 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(N)=O]

MS: Calc'd for C₆₂H₉₆N₁₆O₁₆: 1320.72, found [M+H]⁺: 1321.7.

### Example 309: (Compound 309) (SEQ ID NO: 462)

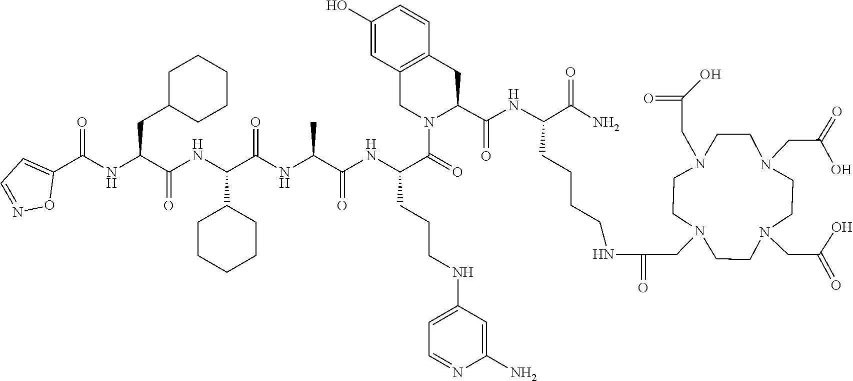 [SMILES: C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(N)=O]

MS: Calc'd for C₆₆H₉₈N₁₆O₁₆: 1370.73, found [M+H]⁺: 1371.7.

### Example 310: (Compound 310)

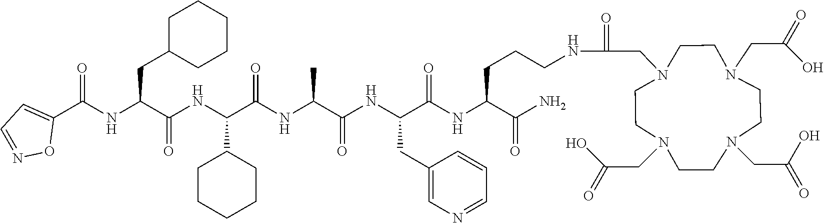 [SMILES: C[C@H](NC(=O)[C@@H](NC(=O)[C@H](CC1CCCCC1)NC(=O)c1ccno1)C1CCCCC1)C(=O)N[C@@H](Cc1cccnc1)C(=O)N[C@@H](CCCNC(=O)CN1CCN(CC(=O)O)CCN(CC(=O)O)CCN(CC(=O)O)CC1)C(N)=O]

MS: Calc'd for C₅₃H₈₁N₁₃O₁₄: 1123.60, found [M+H]⁺: 1124.6.

### Example 311: (Compound 311) (SEQ ID NO: 464)

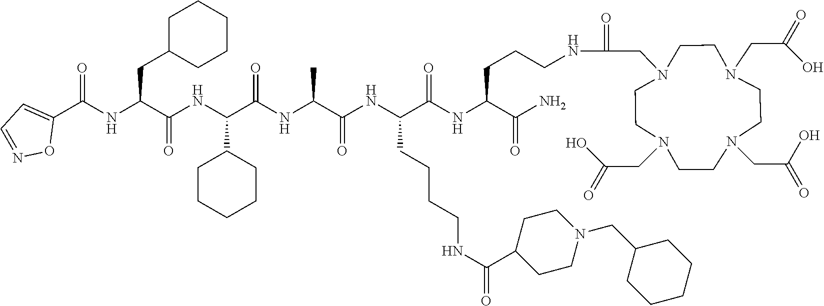 [SMILES: C[C@H](NC(=O)[C@@H](NC(=O)[C@H](CC1CCCCC1)NC(=O)c1ccno1)C1CCCCC1)C(=O)N[C@@H](CCCCNC(=O)C1CCN(CC2CCCCC2)CC1)C(=O)N[C@@H](CCCNC(=O)CN1CCN(CC(=O)O)CCN(CC(=O)O)CCN(CC(=O)O)CC1)C(N)=O]

### Synthesis of N2-(((9H-fluoren-9-yl) methoxy) carbonyl)-N6-(1-benzylpiperidine-4-carbonyl)-L-lysine

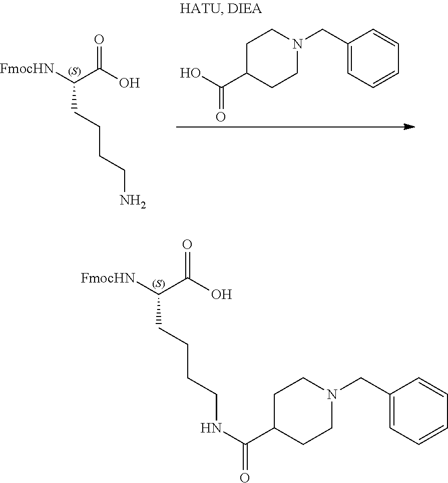

A mixture of (((9H-fluoren-9-yl) methoxy) carbonyl)-L-lysine (3.0 g, 1 Eq, 8.1 mmol), 1-benzylpiperidine-4-carboxylic acid (2.0 g, 1.1 Eq, 9.1 mmol), DIEA (3.2 g, 4.3 mL, 3.0 Eq, 25 mmol), HATU (3.4 g, 1.1 Eq, 8.9 mmol) and DMF (30 mL) were stirred at 25° C. for 2 hours. The crude mixture was directly purified by Prep-HPLC to provide N2-(((9H-fluoren-9-yl) methoxy) carbonyl)-N6-(1-benzylpiperidine-4-carbonyl)-L-lysine (3.0 g, 5.3 mmol, 65%) as a yellow oil. MS: Calc'd for C₃₄H₃₉N₃O₅: 569.29, found [M+H]⁺: 570.4.

Compound 311 was synthesized according to the generic procedures described above to provide the product. MS: Calc'd for C₆₄H₁₀₀N₁₄O₁₅: 1304.75, found [M+H]⁺: 1305.6.

### Example 312: (Compound 312) (SEQ ID NO: 465)

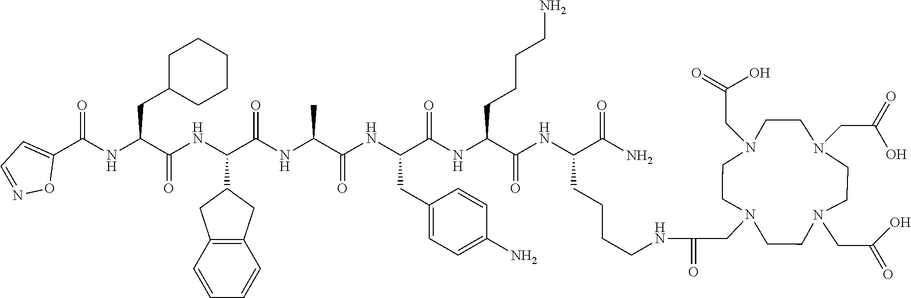 [SMILES: C[C@H](NC(=O)[C@@H](NC(=O)[C@H](CC1CCCCC1)NC(=O)c1ccno1)C1Cc2ccccc2C1)C(=O)N[C@@H](Cc1ccc(N)cc1)C(=O)N[C@@H](CCCCN)C(=O)N[C@@H](CCCCNC(=O)CN1CCN(CC(=O)O)CCN(CC(=O)O)CCN(CC(=O)O)CC1)C(N)=O]

MS: Calc'd for C₆₄H₉₅N₁₅O₁₅: 1313.73, found [M+2H]²⁺: 658.05.

### Example 313: (Compound 313) (SEQ ID NO: 466)

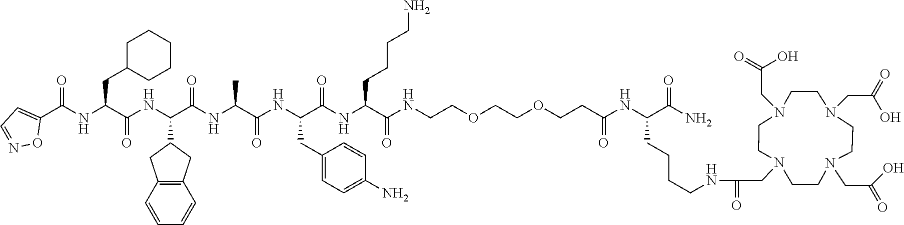 [SMILES: C[C@H](NC(=O)[C@@H](NC(=O)[C@H](CC1CCCCC1)NC(=O)c1ccno1)C1Cc2ccccc2C1)C(=O)N[C@@H](Cc1ccc(N)cc1)C(=O)N[C@@H](CCCCN)C(=O)NCCOCCOCCC(=O)N[C@@H](CCCCNC(=O)CN1CCN(CC(=O)O)CCN(CC(=O)O)CCN(CC(=O)O)CC1)C(N)=O]

MS: Calc'd for C₇₁H₁₀₈N₁₆O₁₈: 1472.80, found [M+3H]³⁺: 492.35.

### Example 314: (Compound 314) (SEQ ID NO: 467)

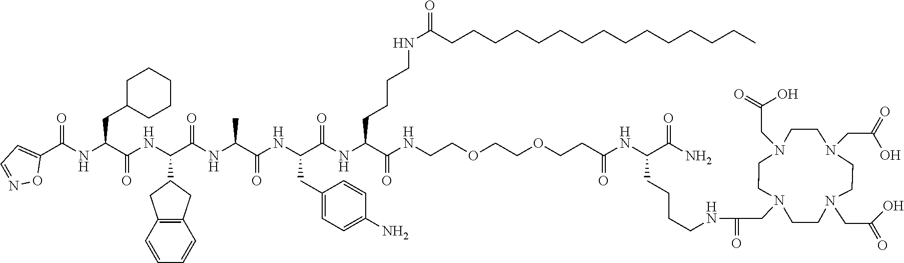 [SMILES: CCCCCCCCCCCCCCCC(=O)NCCCC[C@H](NC(=O)[C@H](Cc1ccc(N)cc1)NC(=O)[C@H](C)NC(=O)[C@@H](NC(=O)[C@H](CC1CCCCC1)NC(=O)c1ccno1)C1Cc2ccccc2C1)C(=O)NCCOCCOCCC(=O)N[C@@H](CCCCNC(=O)CN1CCN(CC(=O)O)CCN(CC(=O)O)CCN(CC(=O)O)CC1)C(N)=O]

MS: Calc'd for C₈₇H₁₃₈N₁₆O₁₉: 1711.03, found [M+3H]³⁺: 571.8.

### Example 315: (Compound 315) (SEQ ID NO: 468)

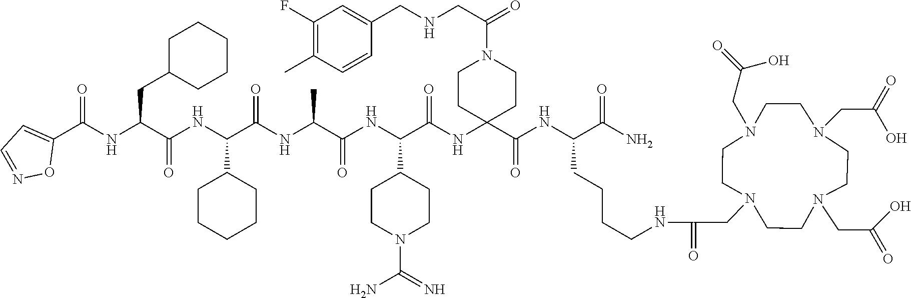 [SMILES: Cc1ccc(CNCC(=O)N2CCC(NC(=O)[C@@H](NC(=O)[C@H](C)NC(=O)[C@@H](NC(=O)[C@H](CC3CCCCC3)NC(=O)c3ccno3)C3CCCCC3)C3CCN(C(=N)N)CC3)(C(=O)N[C@@H](CCCCNC(=O)CN3CCN(CC(=O)O)CCN(CC(=O)O)CCN(CC(=O)O)CC3)C(N)=O)CC2)cc1F]

### Synthesis of 4-((((9H-fluoren-9-yl) methoxy) carbonyl)amino)-1-(N-(tert-butoxycarbonyl)-N-(3-fluoro-4-methylbenzyl)glycyl) piperidine-4-carboxylic acid

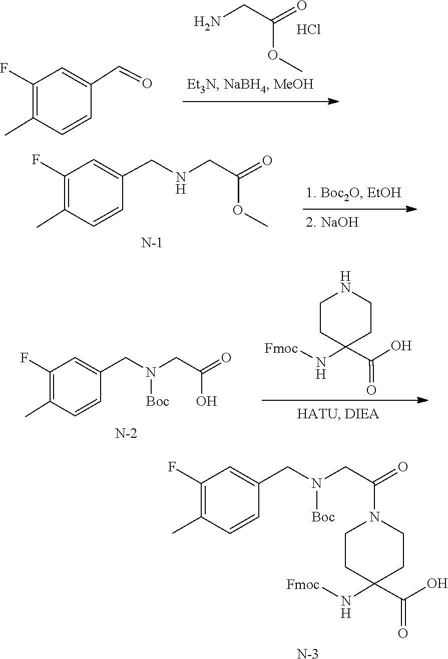

Into a 500 mL round bottom flask was added methyl glycinate hydrochloride (27 g, 2.0 Eq, 0.22 mol), TEA (22 g, 30 mL, 2.0 Eq, 0.22 mol), 3-fluoro-4-methylbenzaldehyde (15 g, 1.0 Eq, 0.11 mol), and MeOH (150 mL). The reaction mixture was stirred at 0° C. for 30 minutes, to which NaBH₄ (8.2 g, 2.0 Eq, 0.22 mol) was added carefully under nitrogen atmosphere at 0° C. The reaction mixture was stirred at 25° C. for an additional 1 hour, and concentrated under reduced pressure. The residue was diluted with water (300 mL), extracted with EtOAc (3× 300 mL), and the combined organic layers were washed with brine (200 mL), dried over anhydrous Na₂SO₄, and concentrated under reduced pressure. The crude residue was purified by MPLC to afford 3-fluoro-4-methylbenzyl)glycinate (N-1, 11.5 g, 54.4 mmol, 50%) as a colorless oil. LCMS: (ESI, m/z): [M+H]⁺=212.3. ¹H NMR (400 MHz, DMSO-d₆) δ 7.19 (t, J=8.0 Hz, 1H), 7.07 (d, J=11.2 Hz, 1H), 7.02 (d, J=8.0 Hz, 1H), 3.69-3.67 (m, 2H), 3.62 (s, 3H), 3.30-3.27 (m, 2H), 2.20 (s, 3H).

Into a 500 mL round bottom flask was added methyl (3-fluoro-4-methylbenzyl)glycinate (N-1, 11.5 g, 1.0 Eq, 54.4 mmol) and EtOH (120 mL), to which was added di-tert-butyl dicarbonate (1.1 g, 1.1 Eq, 5.0 mmol). The reaction mixture was stirred at 25° C. for 2 hours. The mixture was concentrated under reduced pressure to provide methyl N-(tert-butoxycarbonyl)-N-(3-fluoro-4-methylbenzyl)glycinate (17.0 g, 54.6 mmol, 100%) as a white crude solid, which was carried forward without any purification. LCMS: (ESI, m/z): [M+H-Boc]⁺=212.3.

Into a 500 mL round bottom flask was added methyl N-(tert-butoxycarbonyl)-N-(3-fluoro-4-methylbenzyl)glycinate (17 g, 1.0 Eq, 55 mmol) and THF (100 mL). The reaction mixture was stirred at 0° C. for 10 min, after which sodium hydroxide (4.4 g, 2.0 Eq, 0.11 mol) in water (100 mL) was added. The reaction mixture was stirred at 25° C. for 1 hour. The mixture was acidified to pH=5 with 1N HCl solution, diluted with water (200 mL), and extracted with EtOAc (3×300 mL). The combined organic layers were washed with brine (300 mL), dried over anhydrous Na₂SO₄, and concentrated under reduced pressure to provide N-(tert-butoxycarbonyl)-N-(3-fluoro-4-methylbenzyl)glycine (N-2, 15.6 g, 52.5 mmol, 96%) as a colorless oil. LCMS: (ESI, m/z): [M+H-Boc]⁺=198.1.

Into a 500-mL round bottom flask was added N-(tert-butoxycarbonyl)-N-(3-fluoro-4-methylbenzyl)glycine (N-2, 13.2 g, 1.05 Eq, 44.4 mmol), 2-(3H-[1,2,3]triazolo[4,5-b]pyridin-3-yl)-1,1,3,3-tetramethylisouronium hexafluorophosphate (V) (16.8 g, 1.05 Eq, 44.2 mmol), N-ethyl-N-isopropylpropan-2-amine (16.4 g, 3.00 Eq, 127 mmol), and DMF (200 mL). The reaction mixture was stirred at 20° C. for 10 minutes, then 4-((((9H-fluoren-9-yl) methoxy) carbonyl)amino) piperidine-4-carboxylic acid hydrochloride (17.0 g, 1.00 Eq, 42.2 mmol) was added. The reaction mixture was stirred at 25° C. for an additional 2 hours. The mixture was directly purified by MPLC to provide 4-((((9H-fluoren-9-yl) methoxy) carbonyl)amino)-1-(N-(tert-butoxycarbonyl)-N-(3-fluoro-4-methylbenzyl)glycyl) piperidine-4-carboxylic acid (N-3, 11.6 g, 18.0 mmol, 42.6%) as a white solid. LCMS: (ESI, m/z): [M+H]⁺=646.3. ¹H NMR (300 MHz, DMSO-d₆) δ 7.89 (d, J=7.5 Hz, 2H), 7.73 (d, J=7.4 Hz, 2H), 7.67 (s, 1H), 7.42 (td, J=7.4, 1.2 Hz, 2H), 7.33 (t, J=7.4 Hz, 2H), 7.23 (t, J=8.0 Hz, 1H), 7.08-6.95 (m, 2H), 4.45-4.17 (m, 5H), 4.05-3.90 (m, 3H), 3.60-3.50 (m, 1H), 3.20-3.10 (m, 1H), 3.03-2.90 (m, 1H), 2.21 (s, 3H), 2.05-1.95 (m, 2H), 1.88-1.65 (m, 2H), 1.38-1.32 (m, 9H).

MS: Compound 315 was synthesized according to the generic procedures described above to provide the product. Calc'd for CH₁₀₉FN₁₈O₁₆: 1476.83, found [M+2H]²⁺: 739.75.

### Example 316: (Compound 316) (SEQ ID NO: 469)

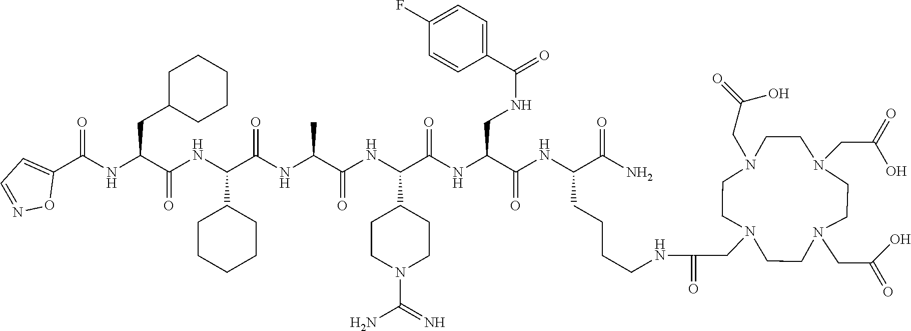 [SMILES: C[C@H](NC(=O)[C@@H](NC(=O)[C@H](CC1CCCCC1)NC(=O)c1ccno1)C1CCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₄H₉₅FN₁₇O₁₆: 1379.74, found [M+H]⁺: 1380.6.

### Example 317: (Compound 317) (SEQ ID NO: 470)

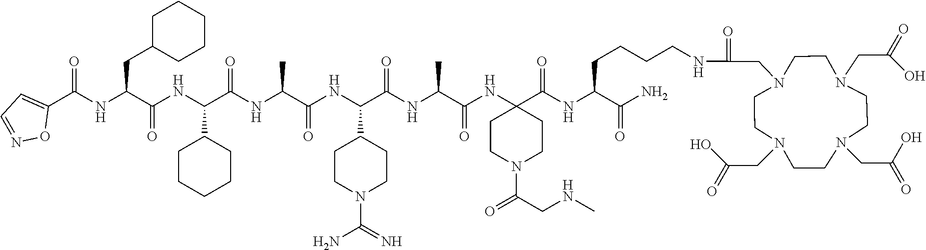 [SMILES: CNCC(=O)N1CCC(NC(=O)[C@H](C)NC(=O)[C@@H](NC(=O)[C@H](C)NC(=O)[C@@H](NC(=O)[C@H](CC2CCCCC2)NC(=O)c2ccno2)C2CCCCC2)C2CCN(C(=N)N)CC2)(C(=O)N[C@@H](CCCCNC(=O)CN2CCN(CC(=O)O)CCN(CC(=O)O)CCN(CC(=O)O)CC2)C(N)=O)CC1]

### Example 318: (Compound 318) (SEQ ID NO: 471)

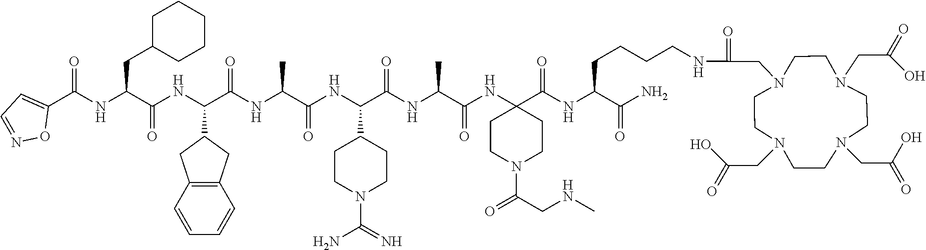 [SMILES: CNCC(=O)N1CCC(NC(=O)[C@H](C)NC(=O)[C@@H](NC(=O)[C@H](C)NC(=O)[C@@H](NC(=O)[C@H](CC2CCCCC2)NC(=O)c2ccno2)C2Cc3ccccc3C2)C2CCN(C(=N)N)CC2)(C(=O)N[C@@H](CCCCNC(=O)CN2CCN(CC(=O)O)CCN(CC(=O)O)CCN(CC(=O)O)CC2)C(N)=O)CC1]

### Example 319: (Compound 319) (SEQ ID NO: 472)

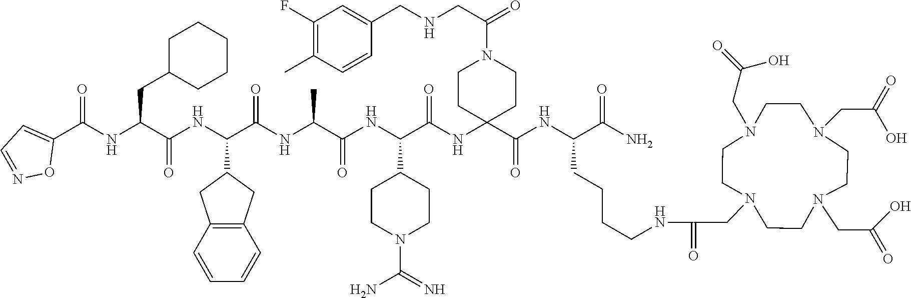 [SMILES: Cc1ccc(CNCC(=O)N2CCC(NC(=O)[C@@H](NC(=O)[C@H](C)NC(=O)[C@@H](NC(=O)[C@H](CC3CCCCC3)NC(=O)c3ccno3)C3Cc4ccccc4C3)C3CCN(C(=N)N)CC3)(C(=O)N[C@@H](CCCCNC(=O)CN3CCN(CC(=O)O)CCN(CC(=O)O)CCN(CC(=O)O)CC3)C(N)=O)CC2)cc1F]

MS: Calc'd for C₇₃H₁₀₇FN₁₈O₁₆: 1510.81, found [M+2H]²⁺: 756.7.

### Example 320: (Compound 320) (SEQ ID NO: 473)

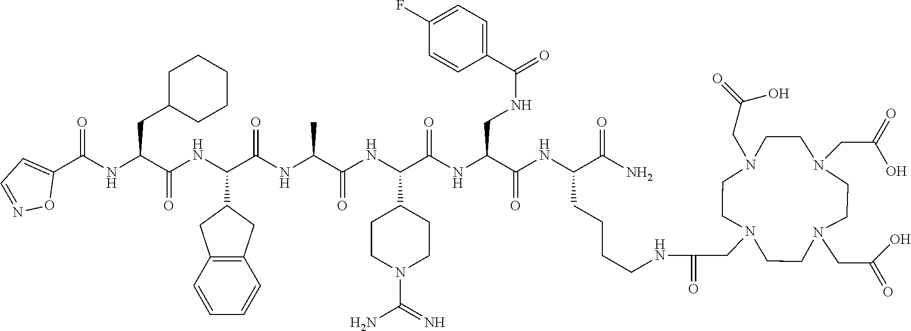 [SMILES: C[C@H](NC(=O)[C@@H](NC(=O)[C@H](CC1CCCCC1)NC(=O)c1ccno1)C1Cc2ccccc2C1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₇H₉₆FN₁₇O₁₆: 1413.72, found [M+2H]²⁺: 708.05.

### Example 321: (Compound 321) (SEQ ID NO: 474)

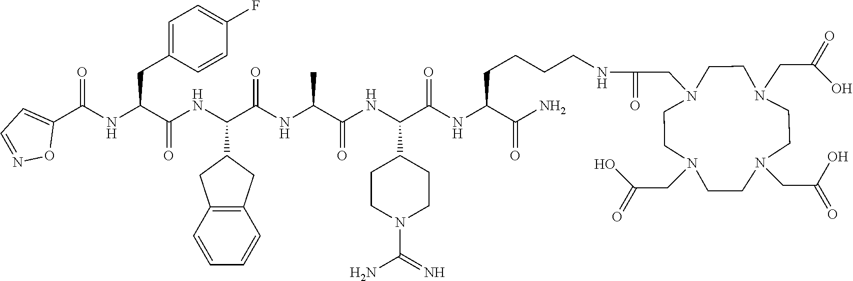 [SMILES: C[C@H](NC(=O)[C@@H](NC(=O)[C@H](Cc1ccc(F)cc1)NC(=O)c1ccno1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₇H₈₀FN₁₅O₁₄: 1217.60, found [M+2H]²⁺: 610.2.

### Example 322: (Compound 322)

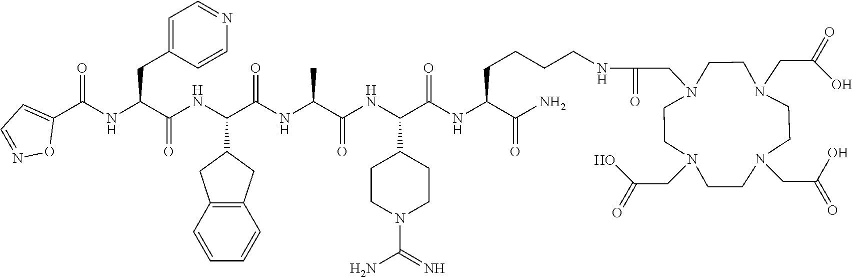 [SMILES: C[C@H](NC(=O)[C@@H](NC(=O)[C@H](Cc1ccncc1)NC(=O)c1ccno1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₆H₈₀N₁₆O₁₄: 1200.60, found [M+2H]²⁺: 601.7.

### Example 323: (Compound 323) (SEQ ID NO: 476)

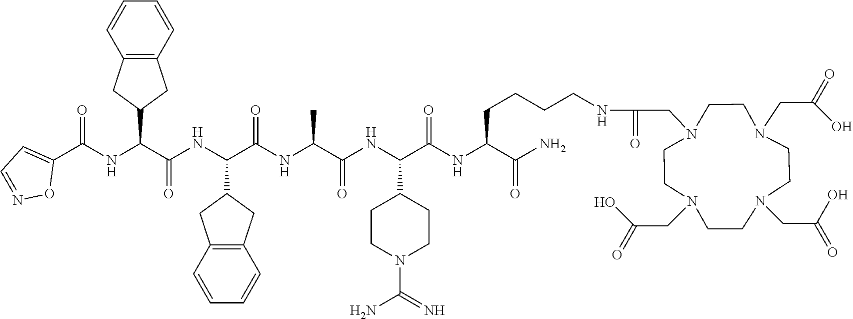 [SMILES: C[C@H](NC(=O)[C@@H](NC(=O)[C@@H](NC(=O)c1ccno1)C1Cc2ccccc2C1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₉H₈₃N₁₅O₁₄: 1225.62, found [M+2H]²⁺: 614.3.

### Example 324: (Compound 324) (SEQ ID NO: 477)

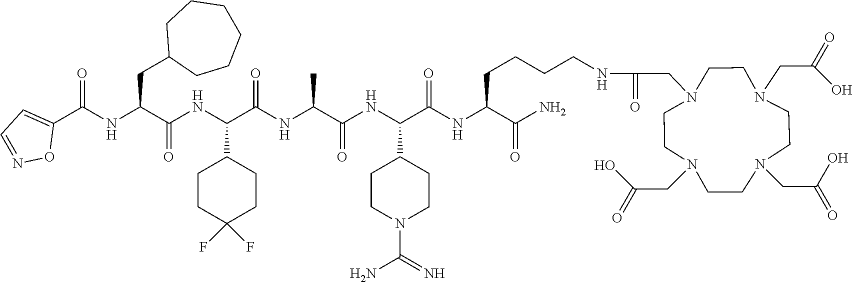 [SMILES: C[C@H](NC(=O)[C@@H](NC(=O)[C@H](CC1CCCCCC1)NC(=O)c1ccno1)C1CCC(F)(F)CC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₅H₈₉F₂N₁₅O₁₄: 1221.67, found [M+2H]²⁺: 612.0.

### Example 325: (Compound 325) (SEQ ID NO: 478)

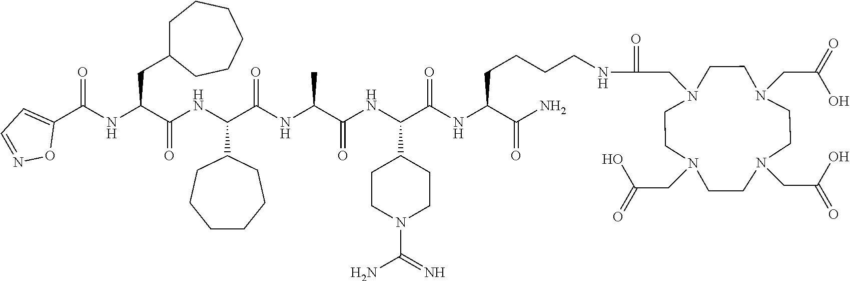 [SMILES: C[C@H](NC(=O)[C@@H](NC(=O)[C@H](CC1CCCCCC1)NC(=O)c1ccno1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₆H₉₃N₁₅O₁₄: 1199.70, found [M+2H]²⁺: 601.0.

### Example 326: (Compound 326) (SEQ ID NO: 479)

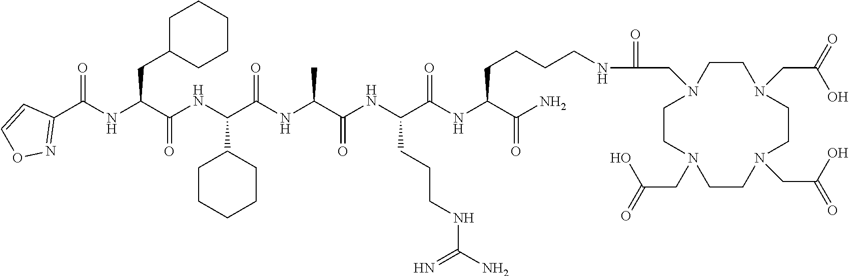 [SMILES: C[C@H](NC(=O)[C@@H](NC(=O)[C@H](CC1CCCCC1)NC(=O)c1ccon1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₈₇N₁₅O₁₄: 1145.66, found [M+3H]³⁺: 383.2.

### Example 327: (Compound 327) (SEQ ID NO: 480)

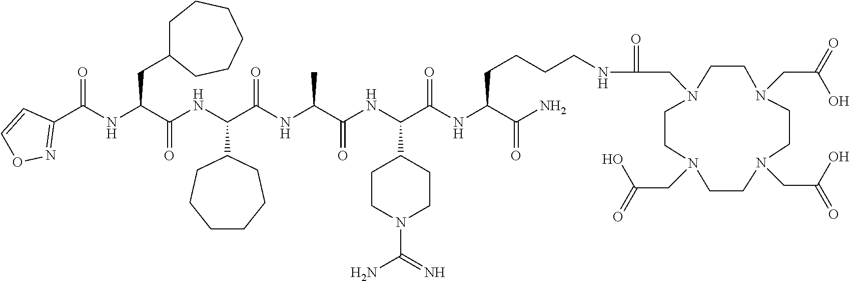 [SMILES: C[C@H](NC(=O)[C@@H](NC(=O)[C@H](CC1CCCCCC1)NC(=O)c1ccon1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₆H₉₃N₁₅O₁₄: 1199.70, found [M+2H]²⁺: 601.0.

### Example 328: (Compound 328) (SEQ ID NO: 481)

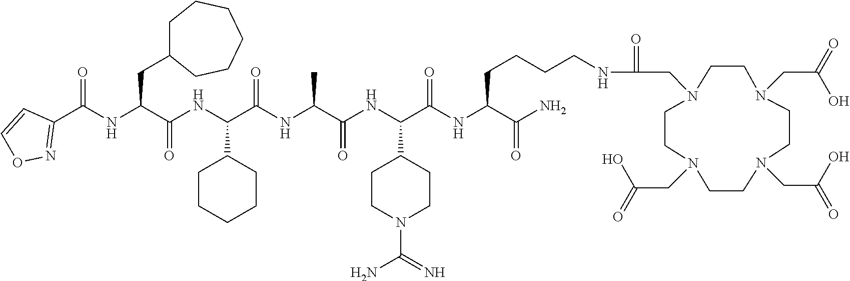 [SMILES: C[C@H](NC(=O)[C@@H](NC(=O)[C@H](CC1CCCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₅H₉₁N₁₅O₁₄: 1185.69, found [M+2H]²⁺: 594.0.

### Example 329: (Compound 329) (SEQ ID NO: 482)

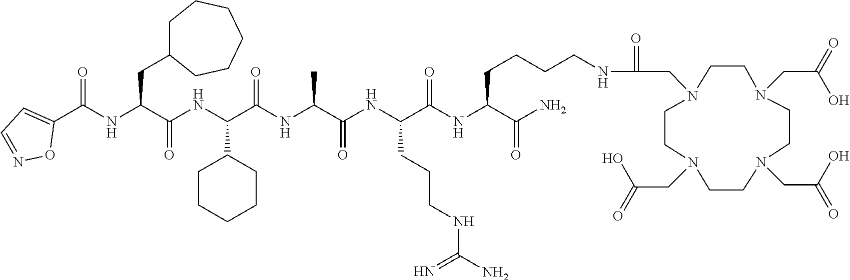 [SMILES: C[C@H](NC(=O)[C@@H](NC(=O)[C@H](CC1CCCCCC1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₃H₃₉N₁₅O₁₄: 1159.67, found [M+3H]³⁺: 387.9.

### Example 330: (Compound 330) (SEQ ID NO: 483)

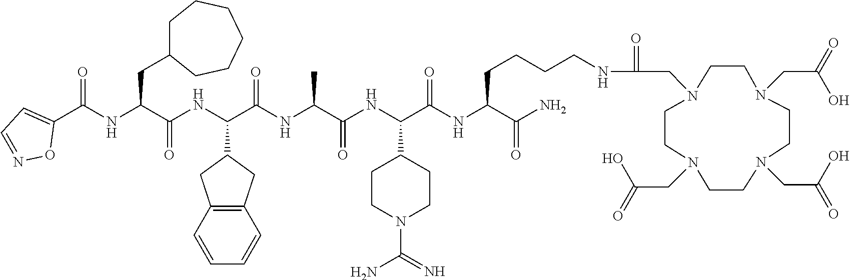 [SMILES: C[C@H](NC(=O)[C@@H](NC(=O)[C@H](CC1CCCCCC1)NC(=O)c1ccno1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₈H₃₉N₁₅O₁₄: 1219.67, found [M+2H]²⁺: 611.0.

### Example 331: (Compound 331) (SEQ ID NO: 484)

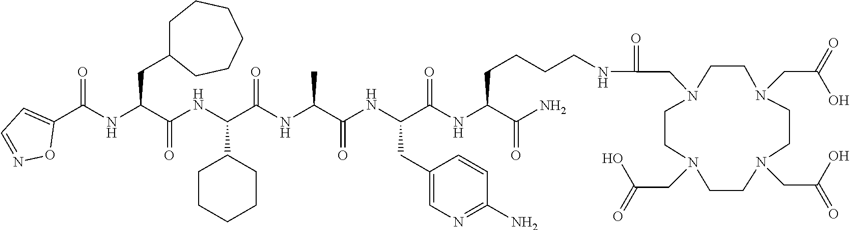 [SMILES: C[C@H](NC(=O)[C@@H](NC(=O)[C@H](CC1CCCCCC1)NC(=O)c1ccno1)C1CCCCC1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(N)=O]

MS: Calc'd for C₅₅H₈₆N₁₄O₁₄: 1166.64, found [M+2H]²⁺: 584.5.

### Example 332: (Compound 332) (SEQ ID NO: 485)

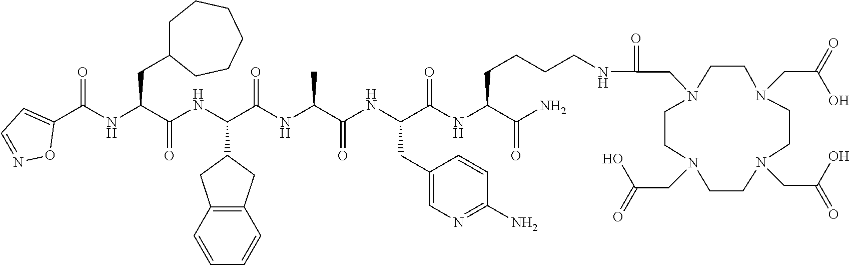 [SMILES: C[C@H](NC(=O)[C@@H](NC(=O)[C@H](CC1CCCCCC1)NC(=O)c1ccno1)C1Cc2ccccc2C1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(N)=O]

MS: Calc'd for C₅₈H₈₄N₁₄O₁₄: 1200.63, found [M+2H]²⁺: 601.6.

### Example 333: (Compound 333) (SEQ ID NO: 486)

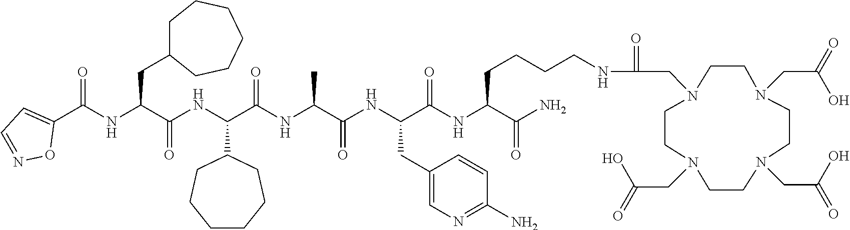 [SMILES: C[C@H](NC(=O)[C@@H](NC(=O)[C@H](CC1CCCCCC1)NC(=O)c1ccno1)C1CCCCCC1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(N)=O]

MS: Calc'd for C₅₆H₈₈N₁₄O₁₄: 1180.66, found [M+2H]²⁺: 591.6.

### Example 334: (Compound 334) (SEQ ID NO: 487)

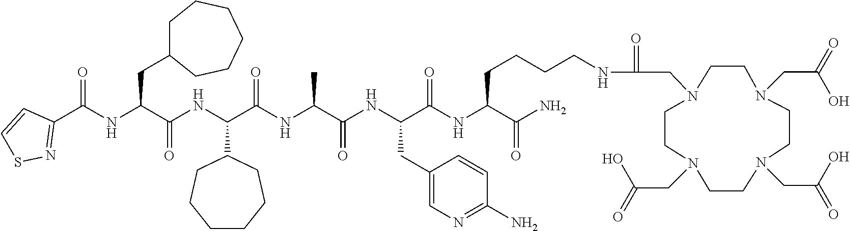 [SMILES: C[C@H](NC(=O)[C@@H](NC(=O)[C@H](CC1CCCCCC1)NC(=O)c1ccsn1)C1CCCCCC1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(N)=O]

MS: Calc'd for C₅₆H₈₈N₁₄O₁₃S: 1196.64, found [M+2H]²⁺: 599.7.

### Example 335: (Compound 335) (SEQ ID NO: 488)

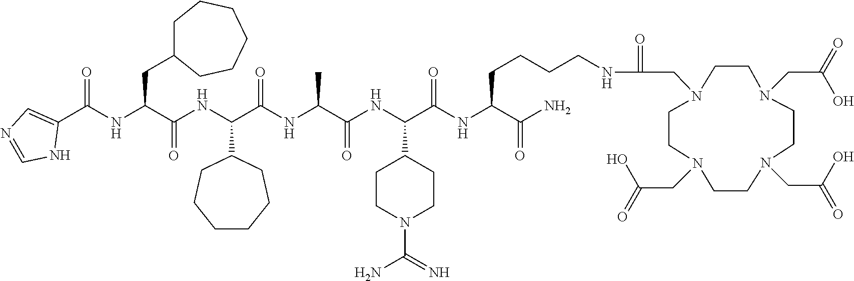 [SMILES: C[C@H](NC(=O)[C@@H](NC(=O)[C@H](CC1CCCCCC1)NC(=O)c1cnc[nH]1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₆H₉₄N₁₆O₁₃: 1198.72, found [M+2H]²⁺: 600.6.

### Example 336: (Compound 336) (SEQ ID NO: 489)

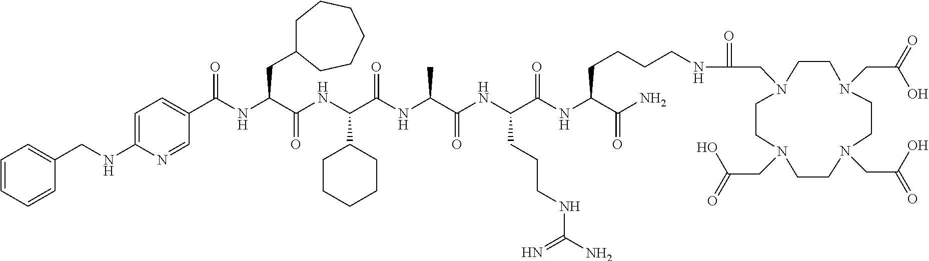 [SMILES: C[C@H](NC(=O)[C@@H](NC(=O)[C@H](CC1CCCCCC1)NC(=O)c1ccc(NCc2ccccc2)nc1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₆₂H₉₈N₁₆O₁₃: 1274.75, found [M+H]⁺: 1275.7.

### Example 337: (Compound 337) (SEQ ID NO: 490)

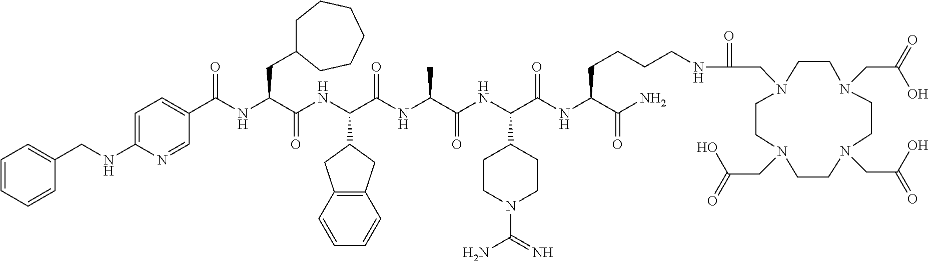 [SMILES: C[C@H](NC(=O)[C@@H](NC(=O)[C@H](CC1CCCCCC1)NC(=O)c1ccc(NCc2ccccc2)nc1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₇H₉₈N₁₆O₁₃: 1334.75, found [M+H]⁺: 1335.9.

### Example 338: (Compound 338) (SEQ ID NO: 491)

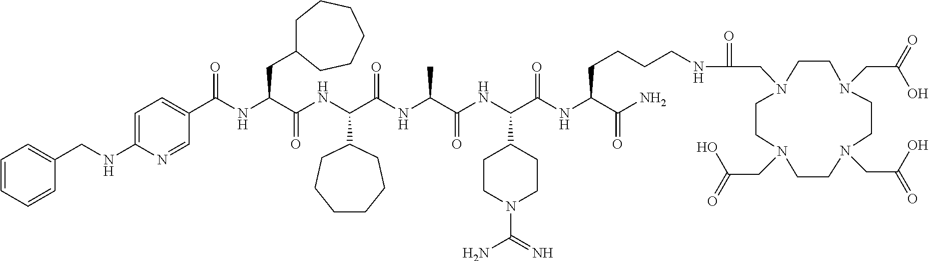 [SMILES: C[C@H](NC(=O)[C@@H](NC(=O)[C@H](CC1CCCCCC1)NC(=O)c1ccc(NCc2ccccc2)nc1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

### Example 339: (Compound 339) (SEQ ID NO: 492)

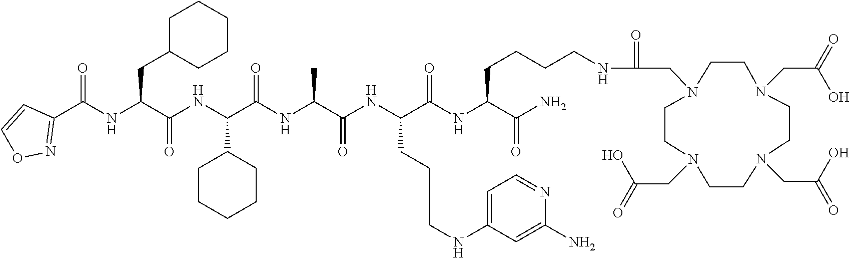 [SMILES: C[C@H](NC(=O)[C@@H](NC(=O)[C@H](CC1CCCCC1)NC(=O)c1ccon1)C1CCCCC1)C(=O)N[C@@H](CCCNc1ccnc(N)c1)C(=O)N[C@@H](CCCCNC(=O)CN1CCN(CC(=O)O)CCN(CC(=O)O)CCN(CC(=O)O)CC1)C(N)=O]

MS: Calc'd for C₅₇H₉₁N₁₅O₁₄: 1195.67, found [M+H]⁺: 1196.8.

### Example 340: (Compound 340) (SEQ ID NO: 493)

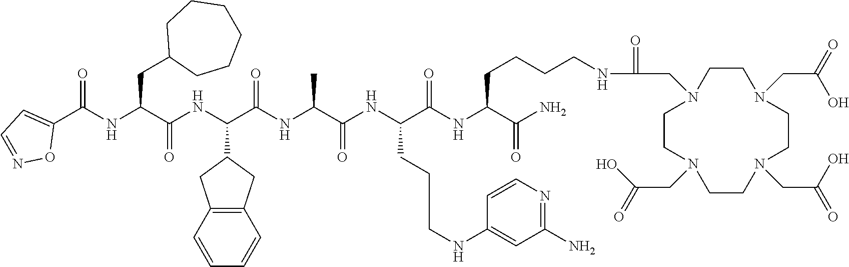 [SMILES: C[C@H](NC(=O)[C@@H](NC(=O)[C@H](CC1CCCCCC1)NC(=O)c1ccno1)C1Cc2ccccc2C1)C(=O)N[C@@H](CCCNc1ccnc(N)c1)C(=O)N[C@@H](CCCCNC(=O)CN1CCN(CC(=O)O)CCN(CC(=O)O)CCN(CC(=O)O)CC1)C(N)=O]

MS: Calc'd for C₆₀H₈₉N₁₅O₁₄: 1243.67, found [M+H]⁺: 1245.2.

### Example 341: (Compound 341) (SEQ ID NO: 494)

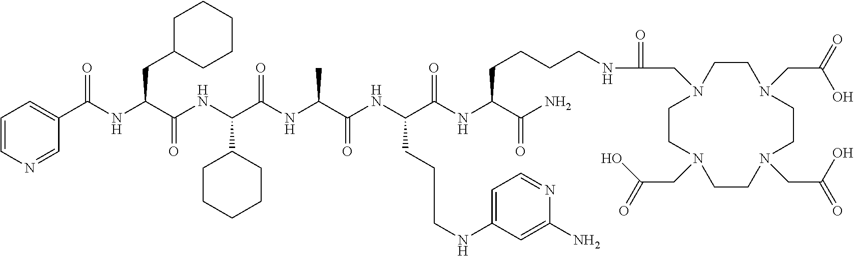 [SMILES: C[C@H](NC(=O)[C@@H](NC(=O)[C@H](CC1CCCCC1)NC(=O)c1cccnc1)C1CCCCC1)C(=O)N[C@@H](CCCNc1ccnc(N)c1)C(=O)N[C@@H](CCCCNC(=O)CN1CCN(CC(=O)O)CCN(CC(=O)O)CCN(CC(=O)O)CC1)C(N)=O]

MS: Calc'd for C₅₈H₉₁N₁₅O₁₃: 1205.69, found [M+H]⁺: 1207.3.

### Example 342: (Compound 342) (SEQ ID NO: 495)

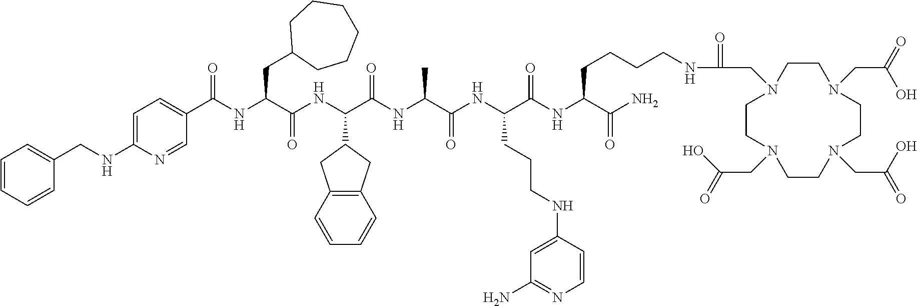 [SMILES: C[C@H](NC(=O)[C@@H](NC(=O)[C@H](CC1CCCCCC1)NC(=O)c1ccc(NCc2ccccc2)nc1)C1Cc2ccccc2C1)C(=O)N[C@@H](CCCNc1ccnc(N)c1)C(=O)N[C@@H](CCCCNC(=O)CN1CCN(CC(=O)O)CCN(CC(=O)O)CCN(CC(=O)O)CC1)C(N)=O]

MS: Calc'd for C₆₉H₉₈N₁₆O₁₃: 1358.75, found [M+H]⁺: 1359.8.

### Example 343: (Compound 343) (SEQ ID NO: 496)

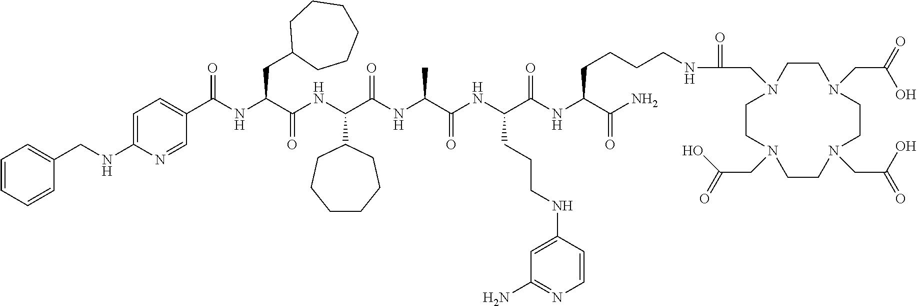 [SMILES: C[C@H](NC(=O)[C@@H](NC(=O)[C@H](CC1CCCCCC1)NC(=O)c1ccc(NCc2ccccc2)nc1)C1CCCCCC1)C(=O)N[C@@H](CCCNc1ccnc(N)c1)C(=O)N[C@@H](CCCCNC(=O)CN1CCN(CC(=O)O)CCN(CC(=O)O)CCN(CC(=O)O)CC1)C(N)=O]

### Example 344: (Compound 344) (SEQ ID NO: 497)

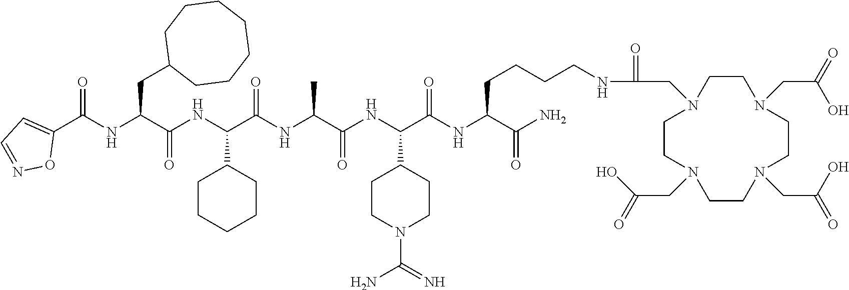 [SMILES: C[C@H](NC(=O)[C@@H](NC(=O)[C@H](CC1CCCCCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₆H₉₃N₁₅O₁₄: 1199.70, found [M+2H]²⁺: 601.1.

### Example 345: (Compound 345) (SEQ ID NO: 498)

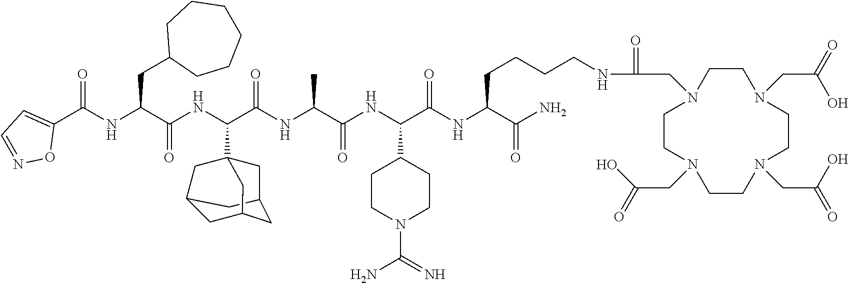 [SMILES: C[C@H](NC(=O)[C@@H](NC(=O)[C@H](CC1CCCCCC1)NC(=O)c1ccno1)C12CC3CC(CC(C3)C1)C2)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₉H₉₅N₁₅O₁₅: 1237.72, found [M+2H]²⁺: 620.1.

### Example 346: (Compound 346) (SEQ ID NO: 499)

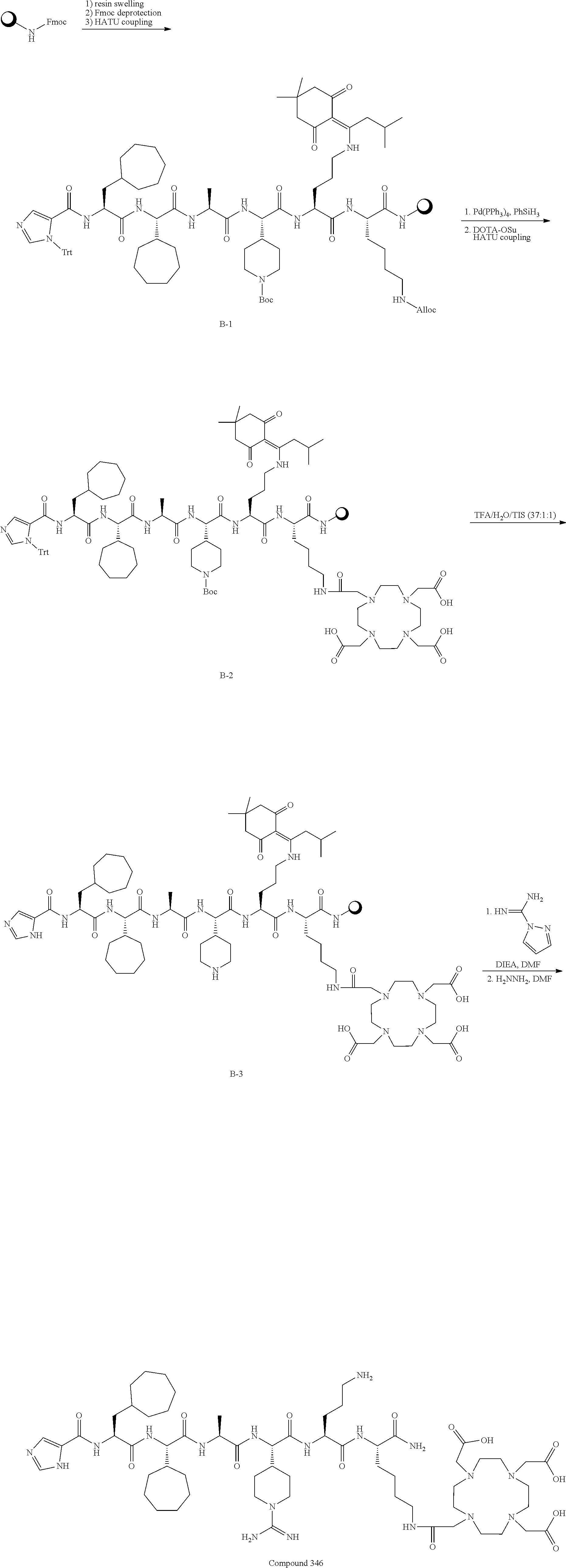

Steps 1-3: Peptidyl resin B-1 on resin was prepared by standard Fmoc-based SPPS procedures from Sieber resin (0.60 g, 0.71 mmol/g, 0.426 mmol) (General SPPS procedure 1-3).

Step 4: Peptidyl resin B-1 on resin was treated with Pd(PPh₃) ₄ (246.23 mg, 0.213 mmol, 0.50 Eq), phenyl silane (860.52 mg, 8.520 mmol, 20.0 Eq) in dichloroethane (10 mL) at 30° C. under a nitrogen atmosphere. After the reaction mixture was shaken for 2 h, the solvent was removed by filtration, and the resin was washed with dichloroethane (3×10 mL) and NMP (3×10 mL) and CH₂Cl₂ (3× 10 mL) to afford the product peptidyl resin.

Step 5: Peptidyl resin B-2 was prepared from peptidyl resin from Step 2 by standard Fmoc-based SPPS procedures (General SPPS procedure 2-3).

Step 6: Peptide B-2 on resin was incubated with a mixture of TFA/H₂O/TIS (37:1:1 v/v/v, 10 mL) at room temperature. After 1 h, the mixture was filtered, and the filtrate was concentrated under reduced pressure. The crude product B-3 was used in the next step directly without further purification.

Step 7: A solution of peptide B-3 (0.70 g crude, 0.474 mmol, 1.0 Eq), pyrazole-1-carboximidamide hydrochloride (347.14 mg, 5.0 Eq) and DIPEA (612.16 mg, 10.0 Eq) in DM (10 mL) was stirred at room temperature overnight. After the solvent was removed under reduced pressure, the residue was purified by RP-HPLC. The desired fractions were combined, concentrated and lyophilized to afford the product as a white solid (30.00 mg, 4.76% overall yield, 76.9% purity). Calc'd for C₇₄H₁₂₂N₁₈O₁₆ 1518.93, found [M+H]⁺: 1519.0.

Step 8: A solution of Product from Step 7 (28.00 mg, 0.018 mmol, 1.0 Eq) in 2% hydrazine monohydrate in DMF (10 mL) was stirred at room temperature overnight. After the solvent was removed under reduced pressure, the residue was purified by RP-HPLC. The desired fractions were combined, concentrated and lyophilized to afford Compound 346 (10.00 mg, 42.6% yield, 90.1% purity) as a white solid. MS: Calc'd for C₆₁H₁₀₄N₁₈O₁₄: 1312.79, found [M+2H]²⁺: 657.5.

### Example 347: (Compound 347) (SEQ ID NO: 500)

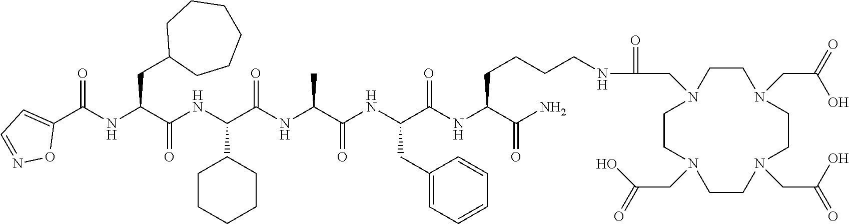 [SMILES: C[C@H](NC(=O)[C@@H](NC(=O)[C@H](CC1CCCCCC1)NC(=O)c1ccno1)C1CCCCC1)C(=O)N[C@@H](Cc1ccccc1)C(=O)N[C@@H](CCCCNC(=O)CN1CCN(CC(=O)O)CCN(CC(=O)O)CCN(CC(=O)O)CC1)C(N)=O]

MS: Calc'd for C₅₆H₈₆N₁₂O₁₄: 1150.64, found [M+H]⁺: 1151.4.

### Example 348: (Compound 348) (SEQ ID NO: 501)

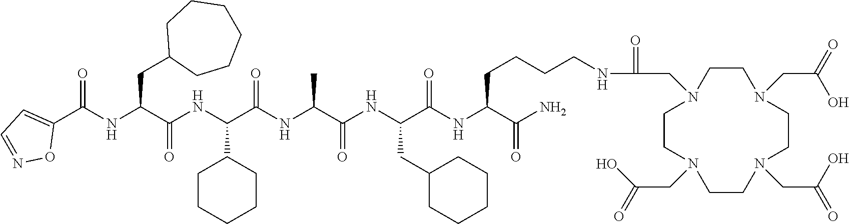 [SMILES: C[C@H](NC(=O)[C@@H](NC(=O)[C@H](CC1CCCCCC1)NC(=O)c1ccno1)C1CCCCC1)C(=O)N[C@@H](CC1CCCCC1)C(=O)N[C@@H](CCCCNC(=O)CN1CCN(CC(=O)O)CCN(CC(=O)O)CCN(CC(=O)O)CC1)C(N)=O]

MS: Calc'd for C₅₆H₉₂N₁₂O₁₄: 1156.69, found [M+H]⁺: 1157.4.

### Example 349: (Compound 349) (SEQ ID NO: 502)

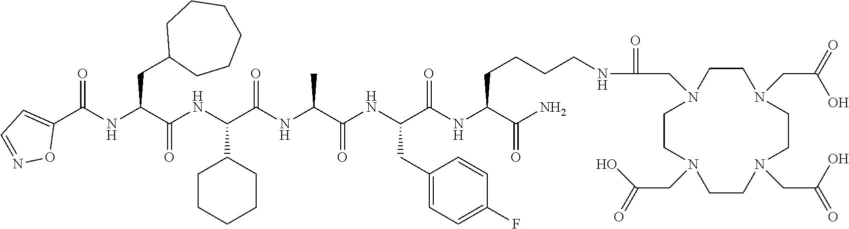 [SMILES: C[C@H](NC(=O)[C@@H](NC(=O)[C@H](CC1CCCCCC1)NC(=O)c1ccno1)C1CCCCC1)C(=O)N[C@@H](Cc1ccc(F)cc1)C(=O)N[C@@H](CCCCNC(=O)CN1CCN(CC(=O)O)CCN(CC(=O)O)CCN(CC(=O)O)CC1)C(N)=O]

MS: Calc'd for C₅₆H₈₅FN₁₂O₁₄: 1168.63, found [M+2H]²⁺: 585.6.

### Example 350: (Compound 350) (SEQ ID NO: 503)

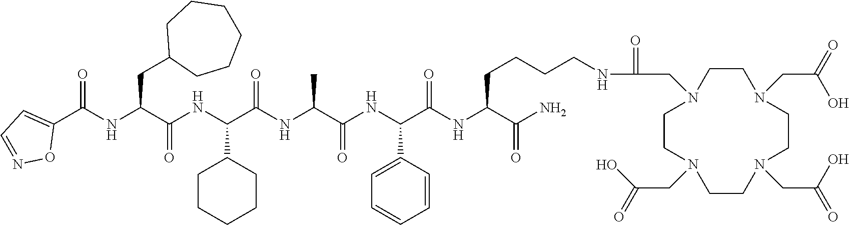 [SMILES: C[C@H](NC(=O)[C@@H](NC(=O)[C@H](CC1CCCCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)c1ccccc1]

MS: Calc'd for C₅₅H₈₄N₁₂O₁₄: 1136.62, found [M+2H]²⁺: 569.6.

### Example 351: (Compound 351) (SEQ ID NO: 504)

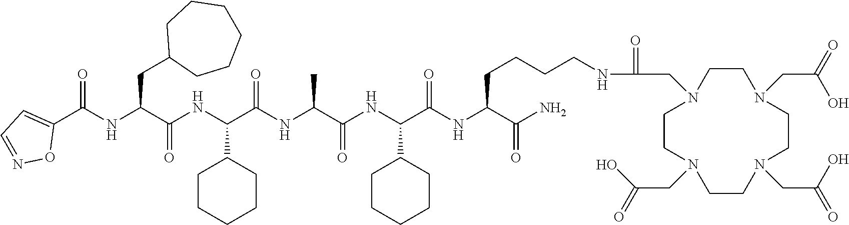 [SMILES: C[C@H](NC(=O)[C@@H](NC(=O)[C@H](CC1CCCCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCCCC1]

MS: Calc'd for C₅₅H₉₀N₁₂O₁₄: 1142.67, found [M+H]⁺: 1143.4.

### Example 352: (Compound 352)

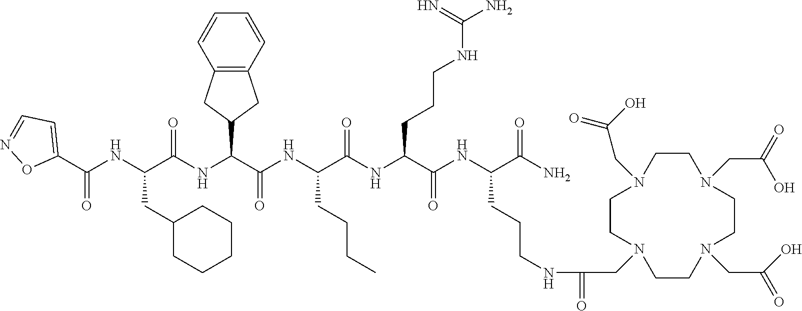 [SMILES: CCCC[C@H](NC(=O)[C@@H](NC(=O)[C@H](CC1CCCCC1)NC(=O)c1ccno1)C1Cc2ccccc2C1)C(=O)N[C@@H](CCCNC(=N)N)C(=O)N[C@@H](CCCNC(=O)CN1CCN(CC(=O)O)CCN(CC(=O)O)CCN(CC(=O)O)CC1)C(N)=O]

MS: Calc'd for C₅₇H₈₉N₁₅O₁₄: 1207.67, found [M+2H]²⁺: 605.1.

### Example 353: (Compound 353) (SEQ ID NO: 506)

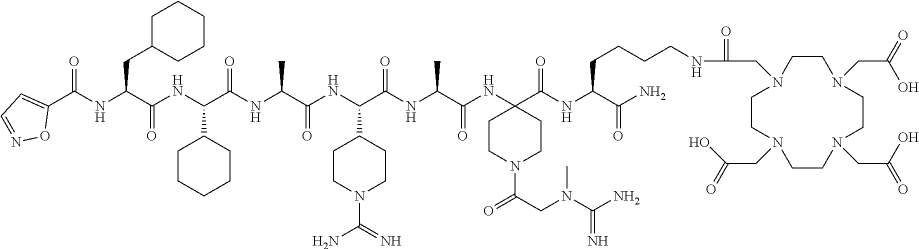 [SMILES: C[C@H](NC(=O)[C@@H](NC(=O)[C@H](C)NC(=O)[C@@H](NC(=O)[C@H](CC1CCCCC1)NC(=O)c1ccno1)C1CCCCC1)C1CCN(C(=N)N)CC1)C(=O)NC1(C(=O)N[C@@H](CCCCNC(=O)CN2CCN(CC(=O)O)CCN(CC(=O)O)CCN(CC(=O)O)CC2)C(N)=O)CCN(C(=O)CN(C)C(=N)N)CC1]

### Example 354: (Compound 354) (SEQ ID NO: 507)

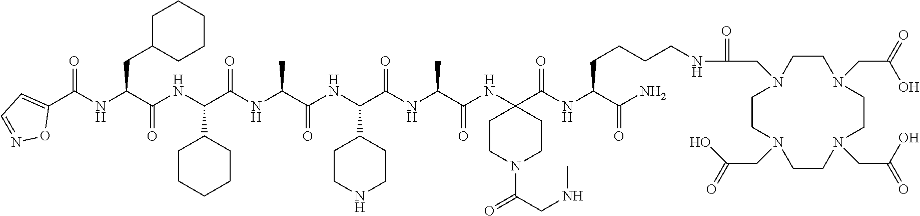 [SMILES: CNCC(=O)N1CCC(NC(=O)[C@H](C)NC(=O)[C@@H](NC(=O)[C@H](C)NC(=O)[C@@H](NC(=O)[C@H](CC2CCCCC2)NC(=O)c2ccno2)C2CCCCC2)C2CCNCC2)(C(=O)N[C@@H](CCCCNC(=O)CN2CCN(CC(=O)O)CCN(CC(=O)O)CCN(CC(=O)O)CC2)C(N)=O)CC1]

MS: Calc'd for C₆₅H₁₀₇N₁₇O₁₇: 1397.67, found [M+2H]²⁺: 700.2.

### Example 355: (Compound 355) (SEQ ID NO: 508)

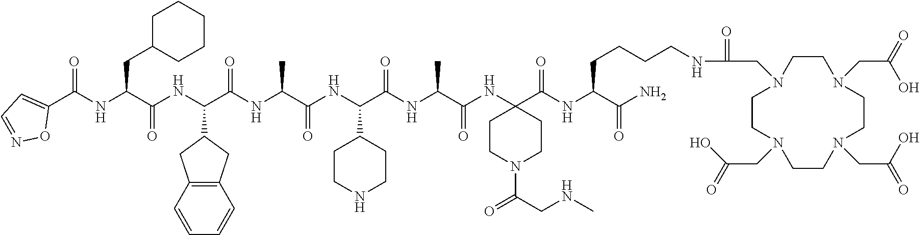 [SMILES: CNCC(=O)N1CCC(NC(=O)[C@H](C)NC(=O)[C@@H](NC(=O)[C@H](C)NC(=O)[C@@H](NC(=O)[C@H](CC2CCCCC2)NC(=O)c2ccno2)C2Cc3ccccc3C2)C2CCNCC2)(C(=O)N[C@@H](CCCCNC(=O)CN2CCN(CC(=O)O)CCN(CC(=O)O)CCN(CC(=O)O)CC2)C(N)=O)CC1]

MS: Calc'd for C₆₈H₁₀₅N₁₇O₁₇: 1431.79, found [M+2H]²⁺: 717.2.

### Example 356: (Compound 356) (SEQ ID NO: 509)

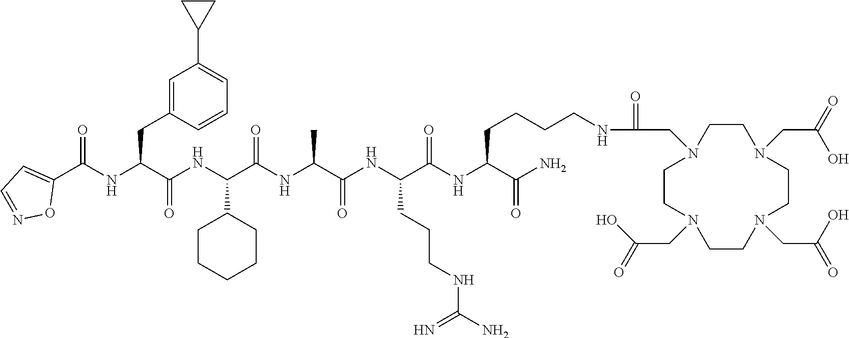 [SMILES: C[C@H](NC(=O)[C@@H](NC(=O)[C@H](Cc1cccc(C2CC2)c1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₅H₈₅N₁₅O₁₄: 1179.64, found [M+2H]²⁺: 591.0.

### Example 357: (Compound 357) (SEQ ID NO: 510)

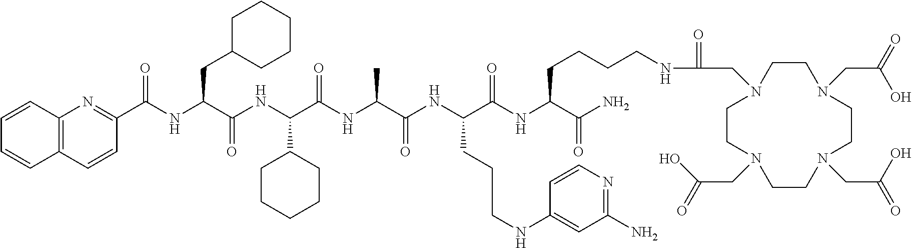 [SMILES: C[C@H](NC(=O)[C@@H](NC(=O)[C@H](CC1CCCCC1)NC(=O)c1ccc2ccccc2n1)C1CCCCC1)C(=O)N[C@@H](CCCNc1ccnc(N)c1)C(=O)N[C@@H](CCCCNC(=O)CN1CCN(CC(=O)O)CCN(CC(=O)O)CCN(CC(=O)O)CC1)C(N)=O]

MS: Calc'd for C₆₂H₉₃N₁₅O₁₃: 1255.71, found [M+H]⁺: 1256.8.

### Example 358: (Compound 358) (SEQ ID NO: 511)

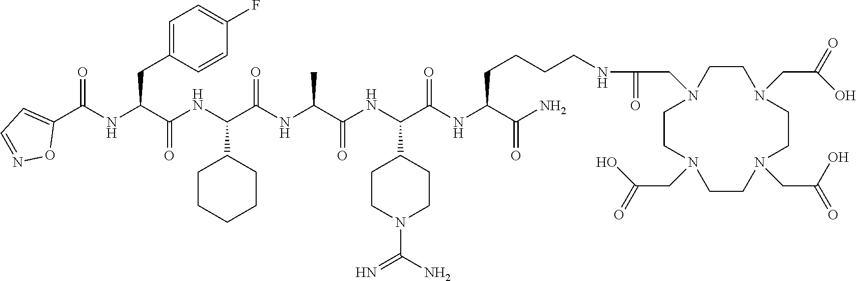 [SMILES: C[C@H](NC(=O)[C@@H](NC(=O)[C@H](Cc1ccc(F)cc1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₄H₈₂FN₁₅O₁₄: 1183.62, found [M+3H]⁺: 395.9.

### Example 359: (Compound 359) (SEQ ID NO: 512)

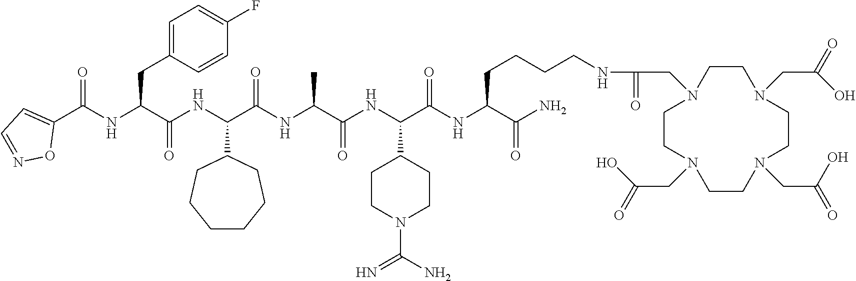 [SMILES: C[C@H](NC(=O)[C@@H](NC(=O)[C@H](Cc1ccc(F)cc1)NC(=O)c1ccno1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₅H₈₄FN₁₅O₁₄: 1197.63, found [M+2H]²⁺: 600.3.

### Example 360: (Compound 360) (SEQ ID NO: 513)

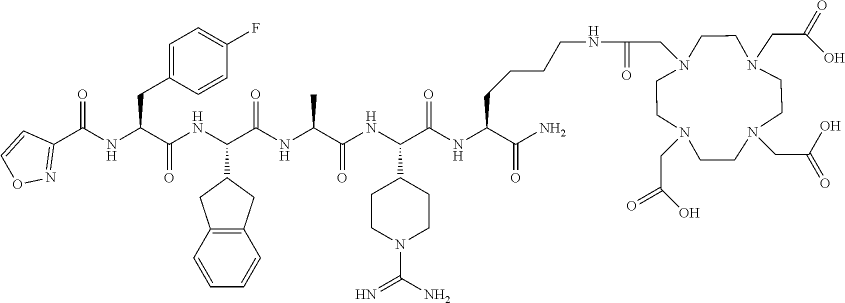 [SMILES: C[C@H](NC(=O)[C@@H](NC(=O)[C@H](Cc1ccc(F)cc1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₇H₃₀FN₁₅O₁₄: 1217.60, found [M+2H]²⁺: 610.2.

### Example 361: (Compound 361) (SEQ ID NO: 514)

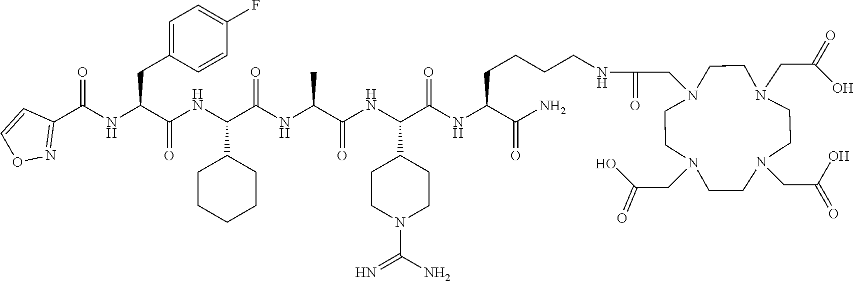 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₄H₈₂FN₁₅O₁₄: 1183.62, found [M+3H]³⁺: 395.8.

### Example 362: (Compound 362) (SEQ ID NO: 515)

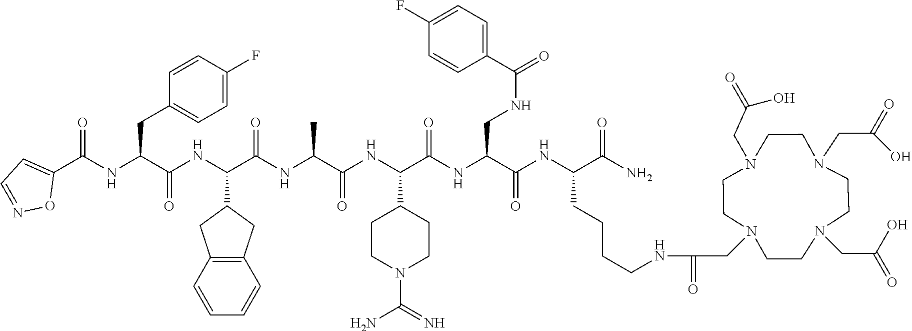 [SMILES: C[C@H](NC(=O)[C@@H](NC(=O)[C@H](Cc1ccc(F)cc1)NC(=O)c1ccno1)C1Cc2ccccc2C1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₇H₈₉F2N₁₇O₁₆: 1425.67, found [M+2H]²⁺: 714.0.

### Example 363: (Compound 363) (SEQ ID NO: 516)

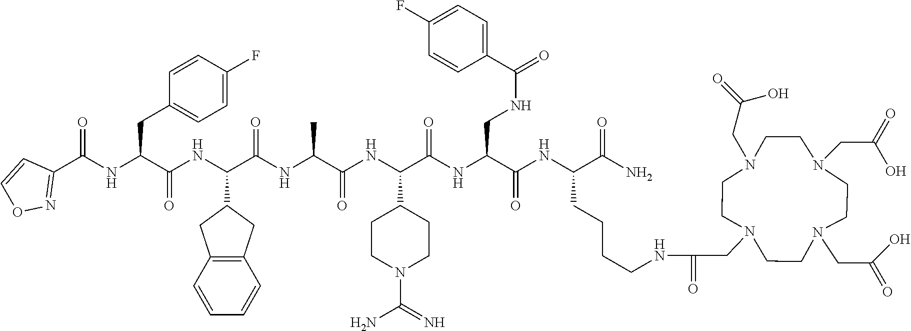 [SMILES: C[C@H](NC(=O)[C@@H](NC(=O)[C@H](Cc1ccc(F)cc1)NC(=O)c1ccon1)C1Cc2ccccc2C1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₇H₈₉F2N₁₇O₁₆: 1425.66, found [M+3H]³⁺: 476.6.

### Example 364: (Compound 364) (SEQ ID NO: 517)

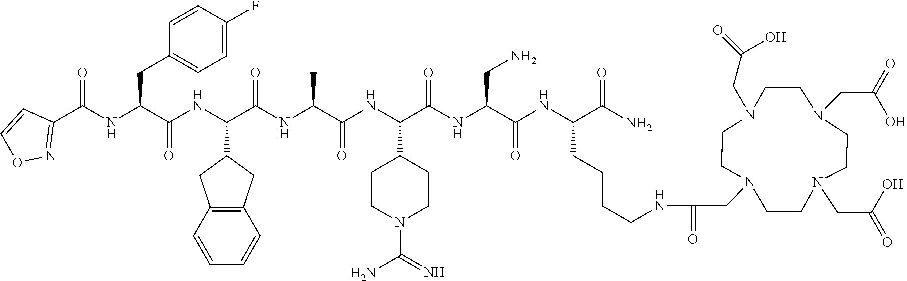 [SMILES: C[C@H](NC(=O)[C@@H](NC(=O)[C@H](Cc1ccc(F)cc1)NC(=O)c1ccon1)C1Cc2ccccc2C1)C(=O)N[C@H](C(=O)N[C@@H](CN)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₀H₈₆FN₁₇O₁₅: 1303.65, found [M+2H]²⁺: 652.9.

### Example 365: (Compound 365) (SEQ ID NO: 518)

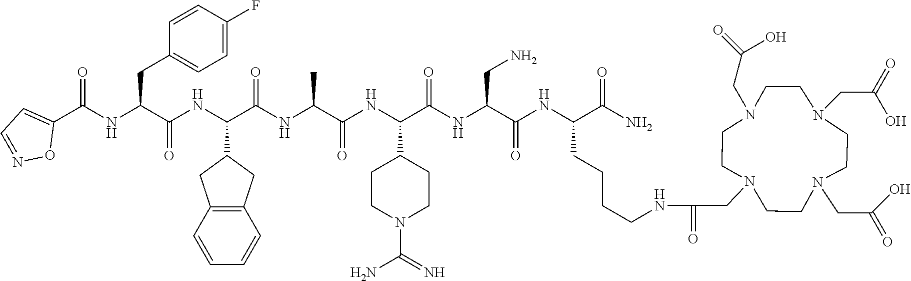 [SMILES: C[C@H](NC(=O)[C@@H](NC(=O)[C@H](Cc1ccc(F)cc1)NC(=O)c1ccno1)C1Cc2ccccc2C1)C(=O)N[C@H](C(=O)N[C@@H](CN)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₀H₈₆FN₁₇O₁₅: 1303.65, found [M+3H]³⁺: 435.8.

### Example 366: (Compound 366)

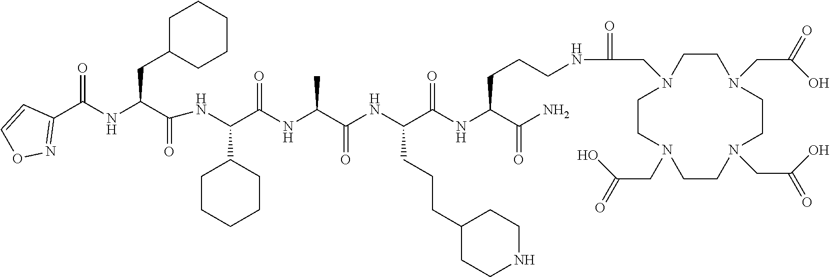 [SMILES: C[C@H](NC(=O)[C@@H](NC(=O)[C@H](CC1CCCCC1)NC(=O)c1ccon1)C1CCCCC1)C(=O)N[C@@H](CCCC1CCNCC1)C(=O)N[C@@H](CCCNC(=O)CN1CCN(CC(=O)O)CCN(CC(=O)O)CCN(CC(=O)O)CC1)C(N)=O]

MS: Calc'd for C₅₅H₉₁N₁₃O₁₄: 1157.68, found [M+H]⁺: 1158.5.

### Example 367: (Compound 367)

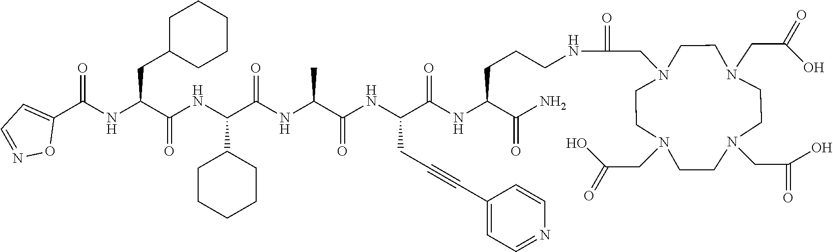 [SMILES: C[C@H](NC(=O)[C@@H](NC(=O)[C@H](CC1CCCCC1)NC(=O)c1ccno1)C1CCCCC1)C(=O)N[C@@H](CC#Cc1ccncc1)C(=O)N[C@@H](CCCNC(=O)CN1CCN(CC(=O)O)CCN(CC(=O)O)CCN(CC(=O)O)CC1)C(N)=O]

### Synthesis of(S)-2-((((9H-fluoren-9-yl) methoxy) carbonyl)amino)-5-(pyridin-4-yl) pent-4-ynoic acid

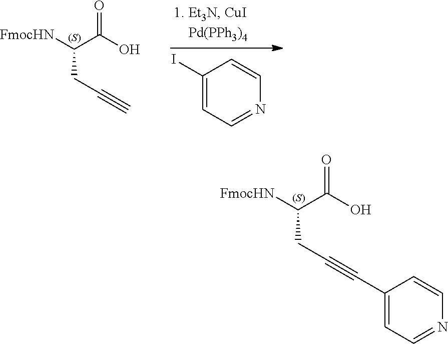

(S)-2-((((9H-fluoren-9-yl) methoxy) carbonyl)amino)-5-(pyridin-4-yl) pent-4-ynoic acid was synthesized from(S)-2-((((9H-fluoren-9-yl) methoxy) carbonyl)amino) pent-4-ynoic acid and 4-iodopyridine in a manner similar to that described in Example 304, Step 1 to provide the product (300 mg, 727 μmol, 12%) as a yellow oil. MS: Calc'd for C₂₅H₂₀N₂O₄: 412.14, found [M+H]⁺: 413.1.

Compound 367 was synthesized according to the generic procedures described above to provide the product. MS: Calc'd for C₅₅H₈₁N₁₃O₁₄: 1147.60, found [M+H]⁺: 1148.5.

### Example 368: (Compound 368)

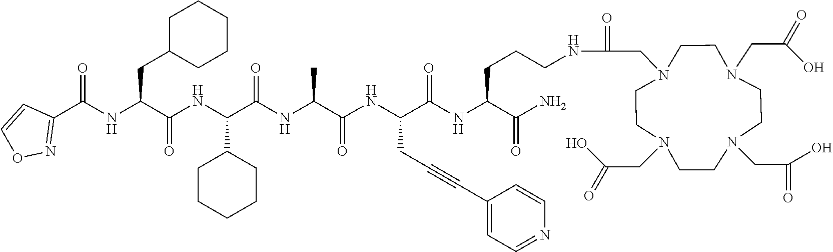 [SMILES: C[C@H](NC(=O)[C@@H](NC(=O)[C@H](CC1CCCCC1)NC(=O)c1ccon1)C1CCCCC1)C(=O)N[C@@H](CC#Cc1ccncc1)C(=O)N[C@@H](CCCNC(=O)CN1CCN(CC(=O)O)CCN(CC(=O)O)CCN(CC(=O)O)CC1)C(N)=O]

MS: Calc'd for C₅₅H₈₁N₁₃O₁₄: 1147.60, found [M+H]⁺: 1148.5.

### Example 369: (Compound 369)

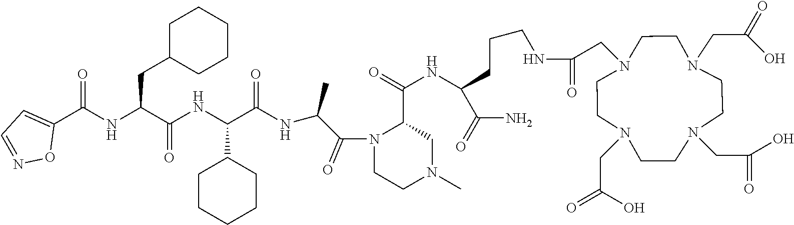 [SMILES: C[C@H](NC(=O)[C@@H](NC(=O)[C@H](CC1CCCCC1)NC(=O)c1ccno1)C1CCCCC1)C(=O)N1CCN(C)C[C@H]1C(=O)N[C@@H](CCCNC(=O)CN1CCN(CC(=O)O)CCN(CC(=O)O)CCN(CC(=O)O)CC1)C(N)=O]

MS: Calc'd for C₅₁H₈₃N₁₃O₁₄: 1101.62, found [M+H]⁺: 1102.5.

### Example 370: (Compound 370)

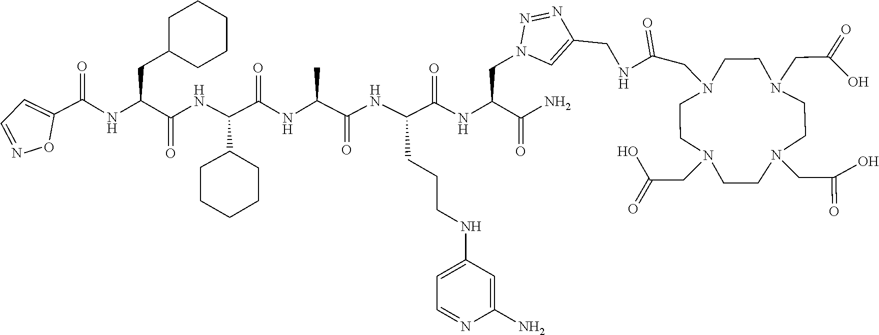 [SMILES: C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N[C@@H](Cn1cc(CNC(=O)CN2CCN(CC(=O)O)CCN(CC(=O)O)CCN(CC(=O)O)CC2)nn1)C(N)=O]

MS: Calc'd for C₅₆H₈₆N₁₈O₁₄: 1234.66, found [M+H]⁺: 1235.6.

### Example 371: (Compound 371)

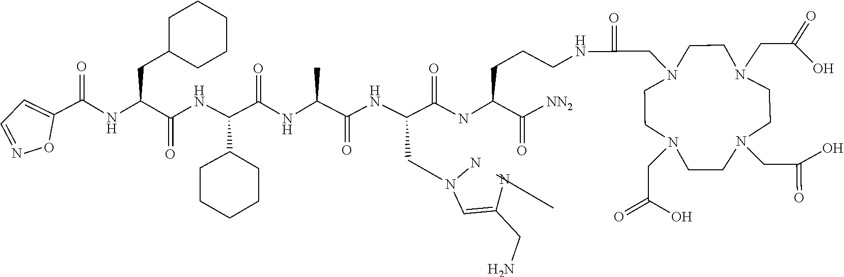

### Synthesis of(S)-2-((((9H-fluoren-9-yl) methoxy) carbonyl)amino)-3-(4-((((benzyloxy) carbonyl)amino)methyl)-1H-1,2,3-triazol-1-yl) propanoic acid

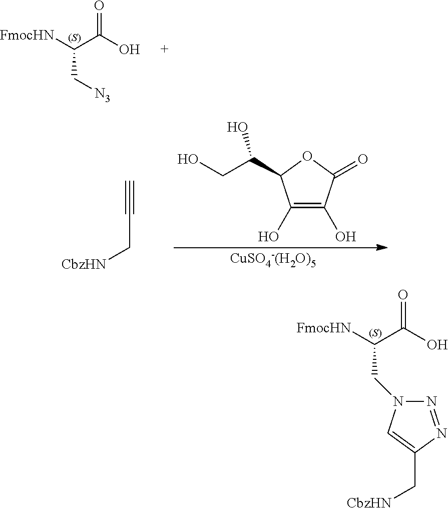

A mixture of(S)-2-((((9H-fluoren-9-yl) methoxy) carbonyl)amino)-3-azidopropanoic acid (1.0 g, 1 Eq, 2.8 mmol), benzyl prop-2-yn-1-ylcarbamate (0.54 g, 1.0 Eq, 2.9 mmol), isoascorbic acid (0.1 g, 0.06 mL, 0.2 Eq, 0.6 mmol), copper (II) sulfate pentahydrate (70 mg, 68 μL, 0.099 Eq, 0.28 mmol), MeOH (10 mL) and H₂O (1 mL) was stirred at 25° C. for 1 hour. The mixture was diluted with water (50 mL), extracted with EtOAc (30 mL×3), the combined organic layers were washed with water (30 mL×2), brine (30 mL), dried over anhydrous Na₂SO₄ and concentrated under reduced pressure. The crude product was purified by MPLC to afford(S)-2-((((9H-fluoren-9-yl) methoxy) carbonyl)amino)-3-(4-((((benzyloxy) carbonyl)amino)methyl)-1H-1,2,3-triazol-1-yl) propanoic acid (1.2 g, 2.2 mmol, 78%) as a yellow solid. MS: Calc'd for C₂₉H₂₇N₅O₆: 541.20, found [M+H]⁺: 542.3.

Compound 371 was synthesized according to the generic procedures described above to provide the product. MS: Calc'd for C₅₁H₈₂N₁₆O₁₄: 1142.62, found [M+H]⁺: 1143.5.

### Example 372: (Compound 372)

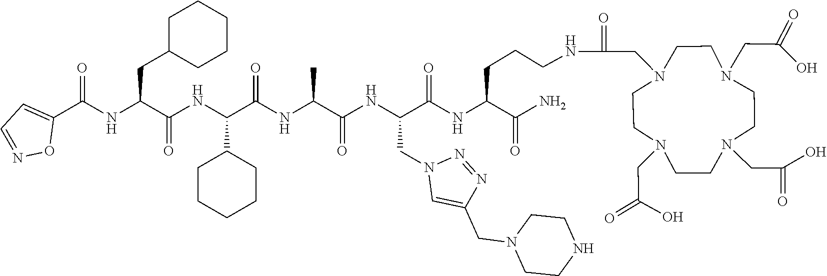 [SMILES: C[C@H](NC(=O)[C@@H](NC(=O)[C@H](CC1CCCCC1)NC(=O)c1ccno1)C1CCCCC1)C(=O)N[C@@H](Cn1cc(CN2CCNCC2)nn1)C(=O)N[C@@H](CCCNC(=O)CN1CCN(CC(=O)O)CCN(CC(=O)O)CCN(CC(=O)O)CC1)C(N)=O]

### Synthesis of(S)-2-((((9H-fluoren-9-yl) methoxy) carbonyl)amino)-3-(4-((4-((benzyloxy) carbonyl) piperazin-1-yl)methyl)-1H-1,2,3-triazol-1-yl) propanoic acid

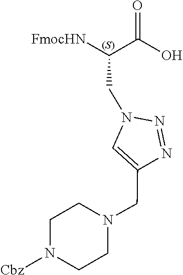

Synthesis of(S)-2-((((9H-fluoren-9-yl) methoxy) carbonyl)amino)-3-(4-((4-((benzyloxy) carbonyl) piperazin-1-yl)methyl)-1H-1,2,3-triazol-1-yl) propanoic acid was synthesized from benzyl 4-(prop-2-yn-1-yl) piperazine-1-carboxylate and(S)-2-((((9H-fluoren-9-yl) methoxy) carbonyl)amino)-3-azidopropanoic acid in a manner similar to Procedure J to provide the product (2.1 g, 3.4 mmol, 81%) as a yellow solid. MS: Calc'd for C₃₃H₃₄N₆O₆: 610.25, found [M+H]⁺: 611.4.

Compound 372 was synthesized according to the generic procedures described above to provide the product. MS: Calc'd for C₅₅H₈₉N₁₇O₁₄: 1211.68, found [M+H]⁺: 1212.5.

### Example 373: (Compound 373)

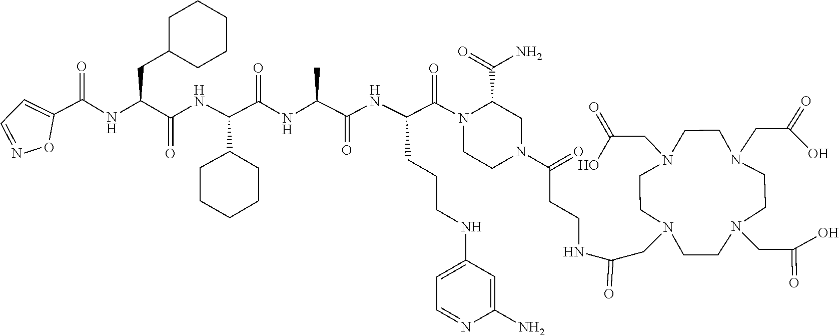 [SMILES: C[C@H](NC(=O)[C@@H](NC(=O)[C@H](CC1CCCCC1)NC(=O)c1ccno1)C1CCCCC1)C(=O)N[C@@H](CCCNc1ccnc(N)c1)C(=O)N1CCN(C(=O)CCNC(=O)CN2CCN(CC(=O)O)CCN(CC(=O)O)CCN(CC(=O)O)CC2)C[C@H]1C(N)=O]

### Example 374: (Compound 374) (SEQ ID NO: 527)

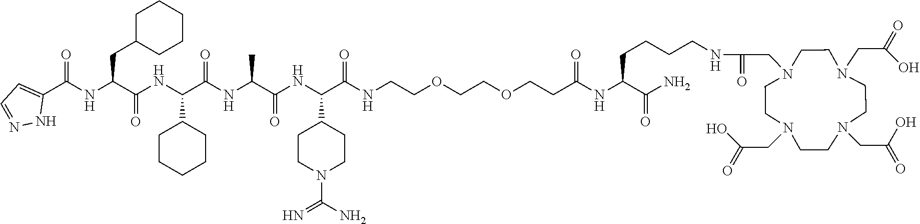 [SMILES: C[C@H](NC(=O)[C@@H](NC(=O)[C@H](CC1CCCCC1)NC(=O)c1ccn[nH]1)C1CCCCC1)C(=O)N[C@H](C(=O)NCCOCCOCCC(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₁H₁₀₃N₁₇O₁₆: 1329.78, found [M+3H]³⁺: 444.6.

### Example 375: (Compound 375) (SEQ ID NO: 528)

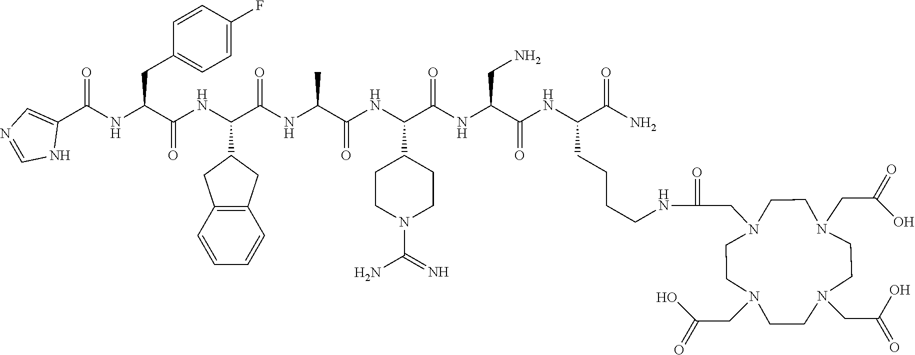 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@H](C(=O)N[C@@H](CN)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₀H₈₇FN₁₈O₁₄: 1302.66, found [M+3H]⁺: 435.6.

### Example 376: (Compound 376) (SEQ ID NO: 529)

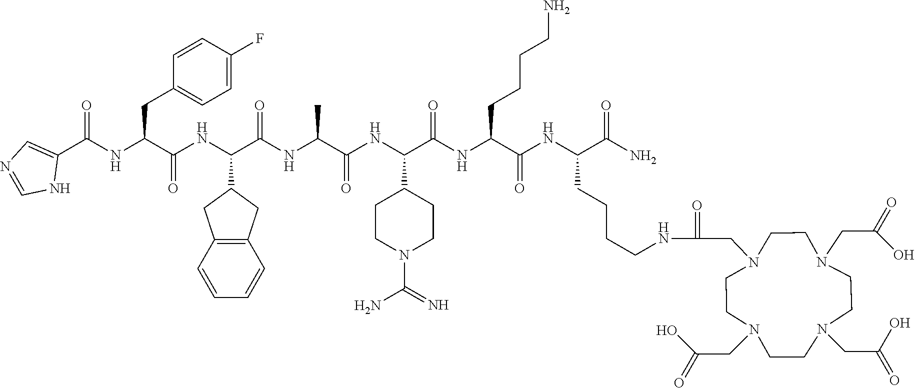 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@H](C(=O)N[C@@H](CCCCN)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₃H₉₃FN₁₈O₁₄: 1344.71, found [M+3H]³⁺: 449.6.

### Example 377: (Compound 377) (SEQ ID NO: 530)

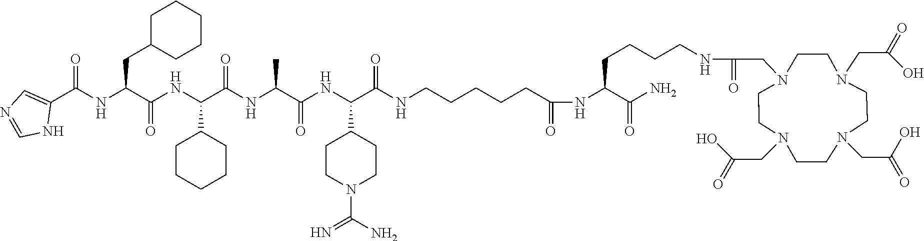 [SMILES: C[C@H](NC(=O)[C@@H](NC(=O)[C@H](CC1CCCCC1)NC(=O)c1cnc[nH]1)C1CCCCC1)C(=O)N[C@H](C(=O)NCCCCCC(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₀H₁₀₁N₁₇O₁₄: 1283.77, found [M+3H]³⁺: 429.3.

### Example 378: (Compound 378) (SEQ ID NO: 531)

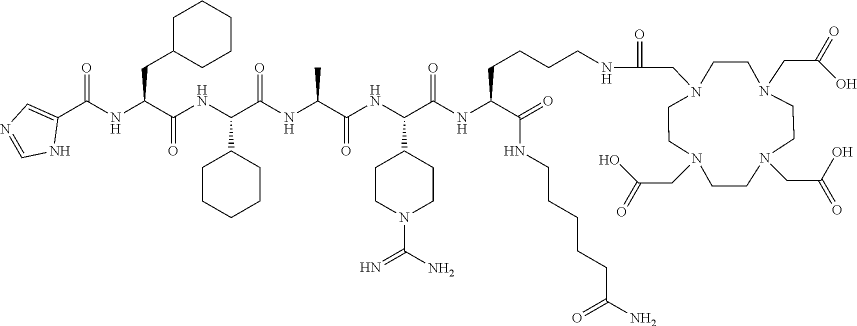 [SMILES: C[C@H](NC(=O)[C@@H](NC(=O)[C@H](CC1CCCCC1)NC(=O)c1cnc[nH]1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₀H₁₀₁N₁₇O₁₄: 1283.77, found [M+3H]³⁺: 429.3.

### Example 379: (Compound 379) (SEQ ID NO: 532)

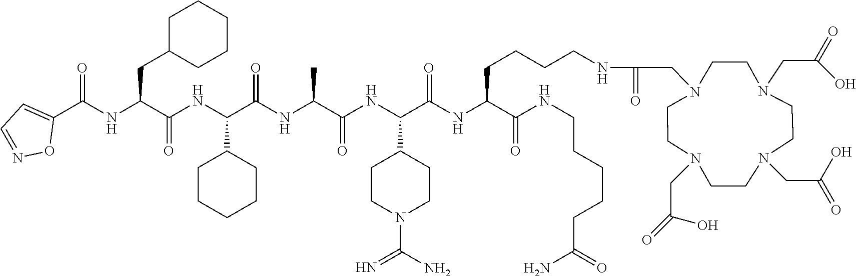 [SMILES: C[C@H](NC(=O)[C@@H](NC(=O)[C@H](CC1CCCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₀H₁₀₀N₁₆O₁₅: 1284.76, found [M+3H]³⁺: 429.6.

### Example 380: (Compound 380) (SEQ ID NO: 533)

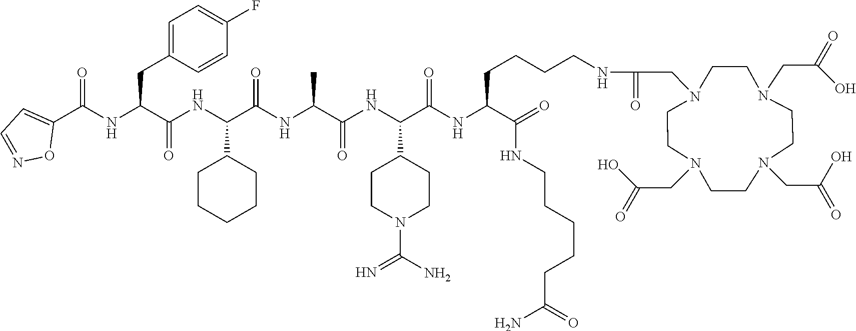 [SMILES: C[C@H](NC(=O)[C@@H](NC(=O)[C@H](Cc1ccc(F)cc1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₆H₉₃FN₁₆O₁₅: 1296.70, found [M+3H]³⁺: 433.6.

### Example 381: (Compound 381) (SEQ ID NO: 534)

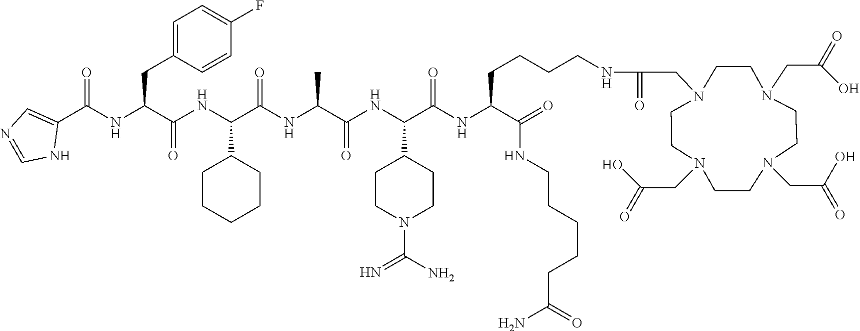 [SMILES: C[C@H](NC(=O)[C@@H](NC(=O)[C@H](Cc1ccc(F)cc1)NC(=O)c1cnc[nH]1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₀H₉₄FN₁₇O₁₄: 1295.72, found [M+3H]³⁺: 433.3.

### Example 382: (Compound 382) (SEQ ID NO: 535)

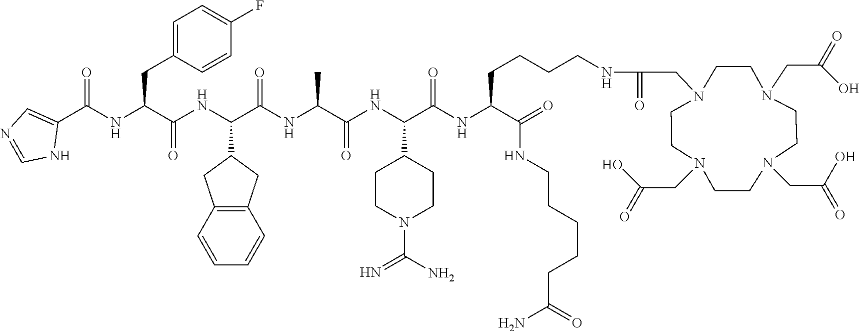 [SMILES: C[C@H](NC(=O)[C@@H](NC(=O)[C@H](Cc1ccc(F)cc1)NC(=O)c1cnc[nH]1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₃H₉₂FN₁₇O₁₄: 1329.70, found [M+3H]⁺: 444.7.

### Example 383: (Compound 383) (SEQ ID NO: 536)

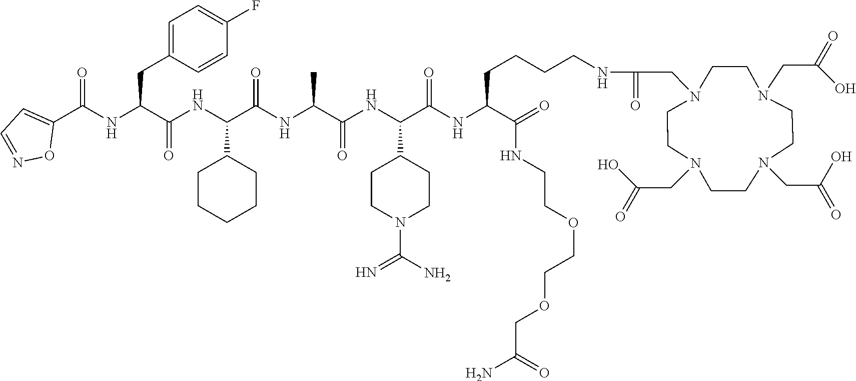 [SMILES: C[C@H](NC(=O)[C@@H](NC(=O)[C@H](Cc1ccc(F)cc1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(N)=O)C1CCN(C(=N)N)CC1]

MS: Calc'd for C₆₀H₉₃FN₁₆O₁₇: 1328.69, found [M+3H]⁺: 444.3.

### Example 384: (Compound 384) (SEQ ID NO: 537)

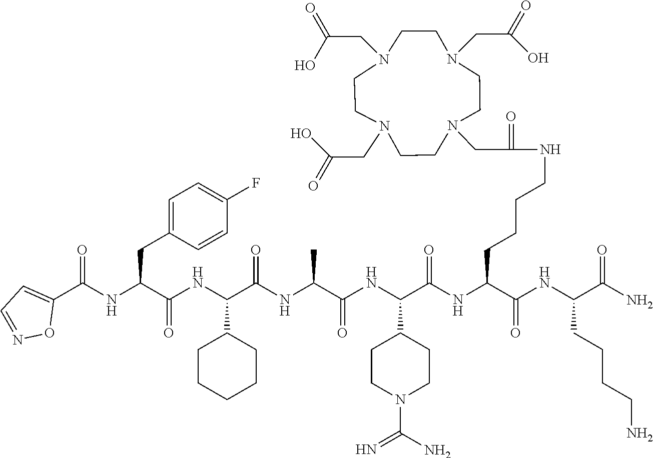 [SMILES: C[C@H](NC(=O)[C@@H](NC(=O)[C@H](Cc1ccc(F)cc1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)N[C@@H](CCCCN)C(N)=O)C1CCN(C(=N)N)CC1]

MS: Calc'd for C₆₀H₉₄FN₁₇O₁₅: 1311.71, found [M+3H]³⁺: 438.6.

### Example 385: (Compound 385) (SEQ ID NO: 538)

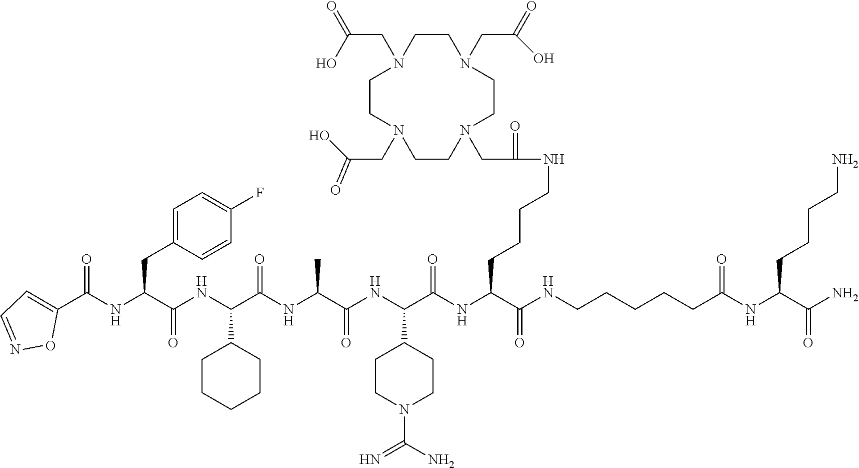 [SMILES: C[C@H](NC(=O)[C@@H](NC(=O)[C@H](Cc1ccc(F)cc1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₆₆H₁₀₅FN₁₈O₁₆: 1424.79, found [M+3H]³⁺: 476.3.

### Example 386: (Compound 386) (SEQ ID NO: 539)

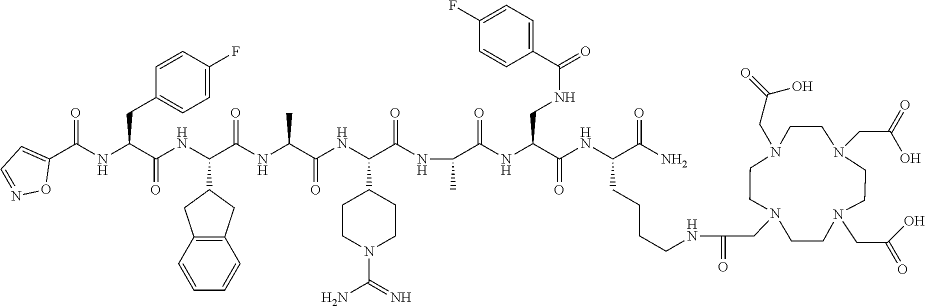 [SMILES: C[C@H](NC(=O)[C@@H](NC(=O)[C@H](C)NC(=O)[C@@H](NC(=O)[C@H](Cc1ccc(F)cc1)NC(=O)c1ccno1)C1Cc2ccccc2C1)C1CCN(C(=N)N)CC1)C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O]

MS: Calc'd for C₇₀H₉₄F2N₁₈O₁₇: 1496.70, found [M+3H]³⁺: 500.3.

### Example 387: (Compound 387) (SEQ ID NO: 540)

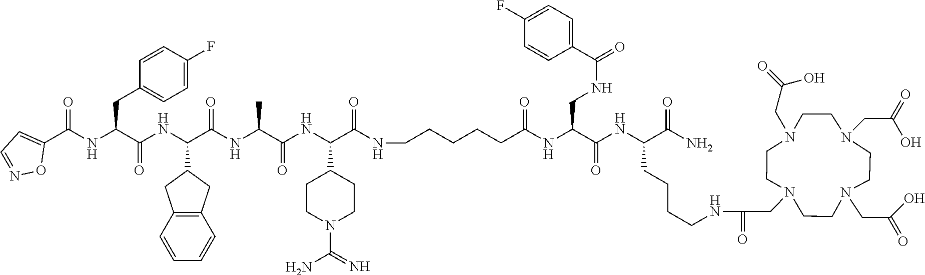 [SMILES: C[C@H](NC(=O)[C@@H](NC(=O)[C@H](Cc1ccc(F)cc1)NC(=O)c1ccno1)C1Cc2ccccc2C1)C(=O)N[C@H](C(=O)NCCCCCC(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₇₃H₁₀₀F₂N₁₈O₁₇: 1538.75, found [M+2H]²⁺: 770.6.

### Example 388: (Compound 388) (SEQ ID NO: 541)

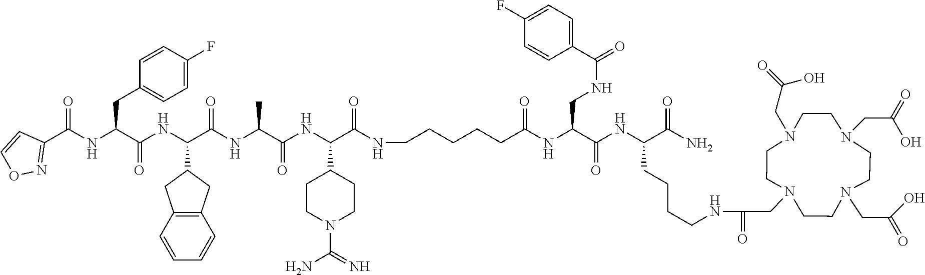 [SMILES: C[C@H](NC(=O)[C@@H](NC(=O)[C@H](Cc1ccc(F)cc1)NC(=O)c1ccon1)C1Cc2ccccc2C1)C(=O)N[C@H](C(=O)NCCCCCC(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₇₃H₁₀₀F₂N₁₈O₁₇: 1538.75, found [M+2H]²⁺: 770.8.

### Example 389: (Compound 389) (SEQ ID NO: 542)

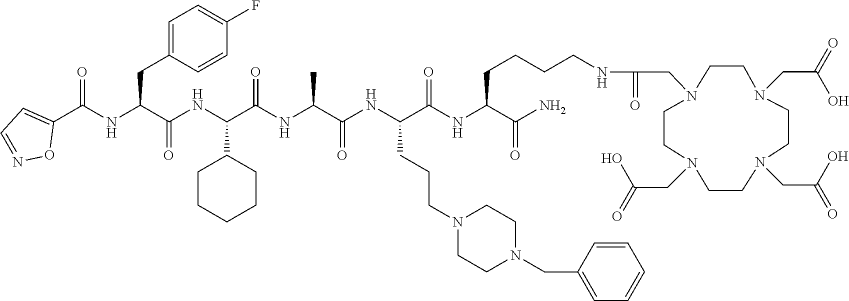 [SMILES: C[C@H](NC(=O)[C@@H](NC(=O)[C@H](Cc1ccc(F)cc1)NC(=O)c1ccno1)C1CCCCC1)C(=O)N[C@@H](CCCN1CCN(Cc2ccccc2)CC1)C(=O)N[C@@H](CCCCNC(=O)CN1CCN(CC(=O)O)CCN(CC(=O)O)CCN(CC(=O)O)CC1)C(N)=O]

### Example 390: (Compound 390)

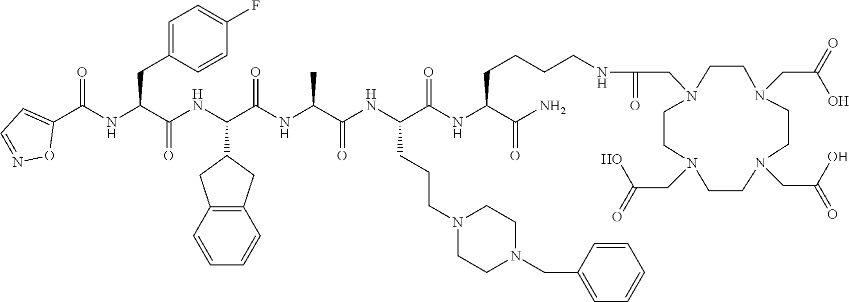 [SMILES: C[C@H](NC(=O)[C@@H](NC(=O)[C@H](Cc1ccc(F)cc1)NC(=O)c1ccno1)C1Cc2ccccc2C1)C(=O)N[C@@H](CCCN1CCN(Cc2ccccc2)CC1)C(=O)N[C@@H](CCCCNC(=O)CN1CCN(CC(=O)O)CCN(CC(=O)O)CCN(CC(=O)O)CC1)C(N)=O]

### Example 391: (Compound 391)

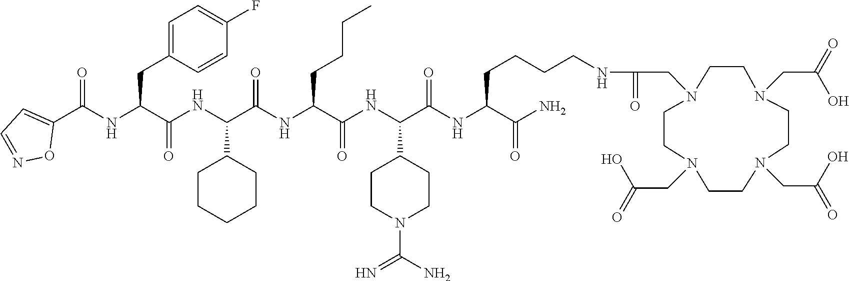 [SMILES: CCCC[C@H](NC(=O)[C@@H](NC(=O)[C@H](Cc1ccc(F)cc1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₇H₈₈FN₁₅O₁₄: 1225.66, found [M+2H]²⁺: 614.3.

### Example 392: (Compound 392)

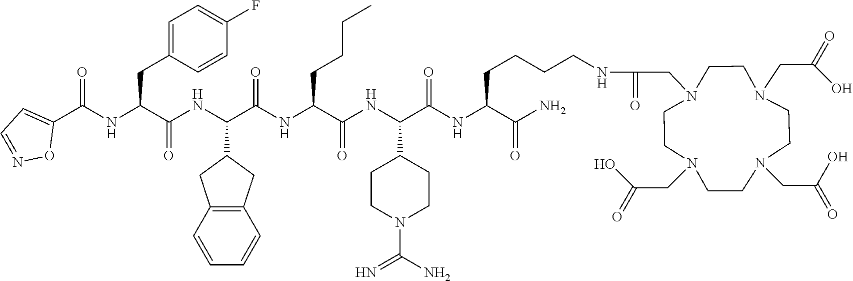 [SMILES: CCCC[C@H](NC(=O)[C@@H](NC(=O)[C@H](Cc1ccc(F)cc1)NC(=O)c1ccno1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₀H₈₆FN₁₅O₁₄: 1259.65, found [M+2H]²⁺: 630.95.

### Example 393: (Compound 393)

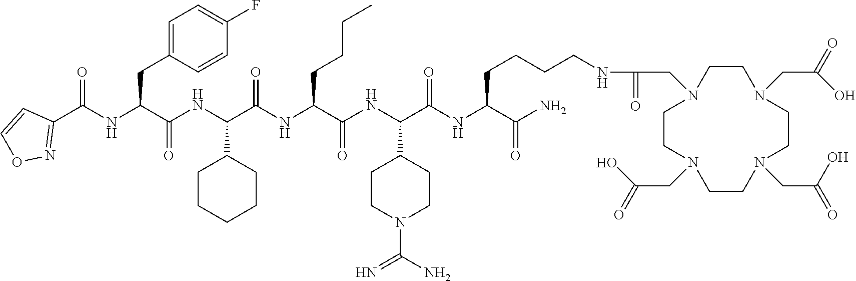 [SMILES: CCCC[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₇H₈₈FN₁₅O₁₄: 1225.66, found [M+2H]²⁺: 613.9.

### Example 394: (Compound 394)

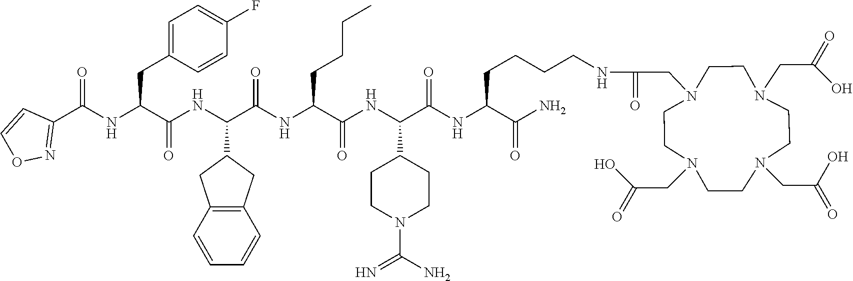 [SMILES: CCCC[C@H](NC(=O)[C@@H](NC(=O)[C@H](Cc1ccc(F)cc1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₀H₈₆FN₁₅O₁₄: 1259.65, found [M+2H]²⁺: 631.0.

### Example 395: (Compound 395) (SEQ ID NO: 548)

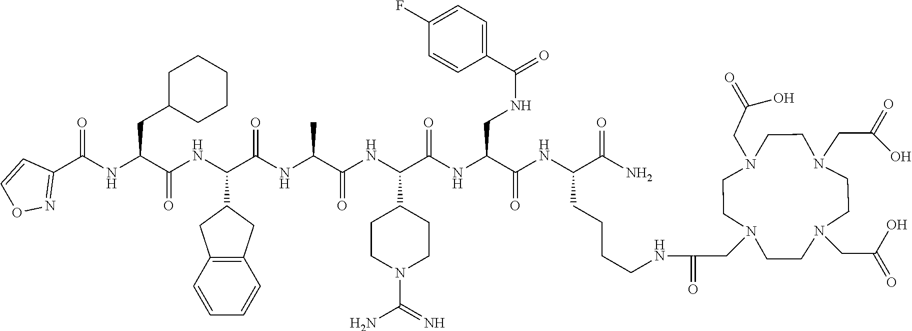 [SMILES: C[C@H](NC(=O)[C@@H](NC(=O)[C@H](CC1CCCCC1)NC(=O)c1ccon1)C1Cc2ccccc2C1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₇H₉₆FN₁₇O₁₆: 1413.72, found [M+3H]³⁺: 472.7.

### Example 396: (Compound 396) (SEQ ID NO: 549)

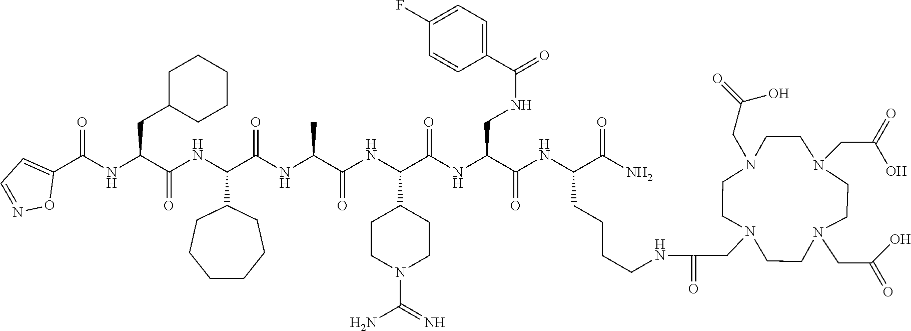 [SMILES: C[C@H](NC(=O)[C@@H](NC(=O)[C@H](CC1CCCCC1)NC(=O)c1ccno1)C1CCCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₅H₁₀₀FN₁₇O₁₆: 1393.75, found [M+3H]³⁺: 465.95.

### Example 397: (Compound 397) (SEQ ID NO: 550)

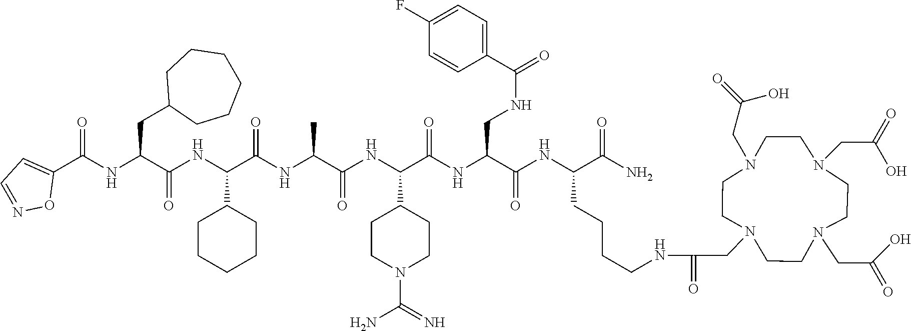 [SMILES: C[C@H](NC(=O)[C@@H](NC(=O)[C@H](CC1CCCCCC1)NC(=O)c1ccno1)C1CCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₅H₁₀₀FN₁₇O₁₆: 1393.75, found [M+3H]³⁺: 465.95.

### Example 398: (Compound 398)

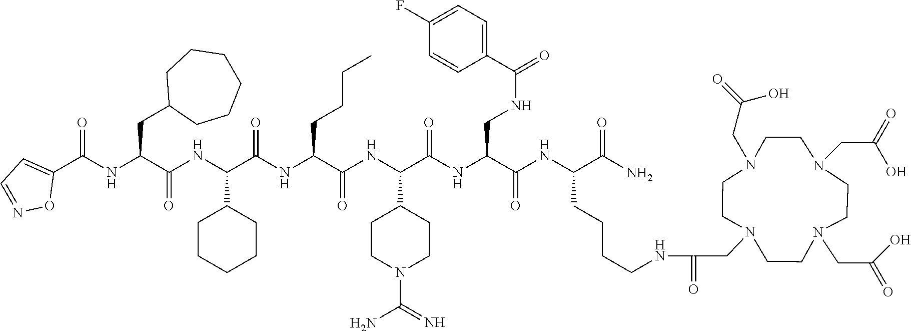 [SMILES: CCCC[C@H](NC(=O)[C@@H](NC(=O)[C@H](CC1CCCCCC1)NC(=O)c1ccno1)C1CCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₈H₁₀₆FN₁₇O₁₆: 1435.80, found [M+3H]³⁺: 479.9.

### Example 399: (Compound 399)

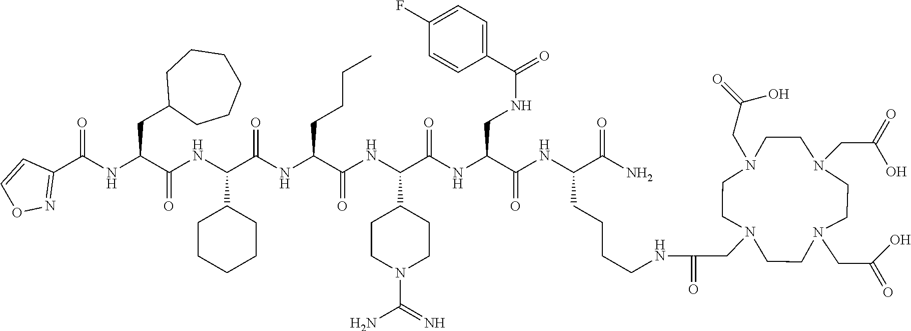 [SMILES: CCCC[C@H](NC(=O)[C@@H](NC(=O)[C@H](CC1CCCCCC1)NC(=O)c1ccon1)C1CCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₈H₁₀₆FN₁₇O₁₆: 1436.70, found [M+2H]²⁺: 719.4.

### Example 400: (Compound 400) (SEQ ID NO: 553)

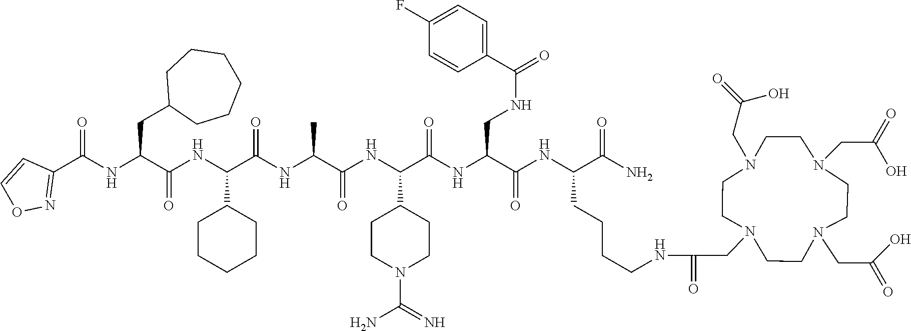 [SMILES: C[C@H](NC(=O)[C@@H](NC(=O)[C@H](CC1CCCCCC1)NC(=O)c1ccon1)C1CCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₅H₁₀₀FN₁₇O₁₆: 1393.75, found [M+3H]³⁺: 465.95.

### Example 401: (Compound 401) (SEQ ID NO: 554)

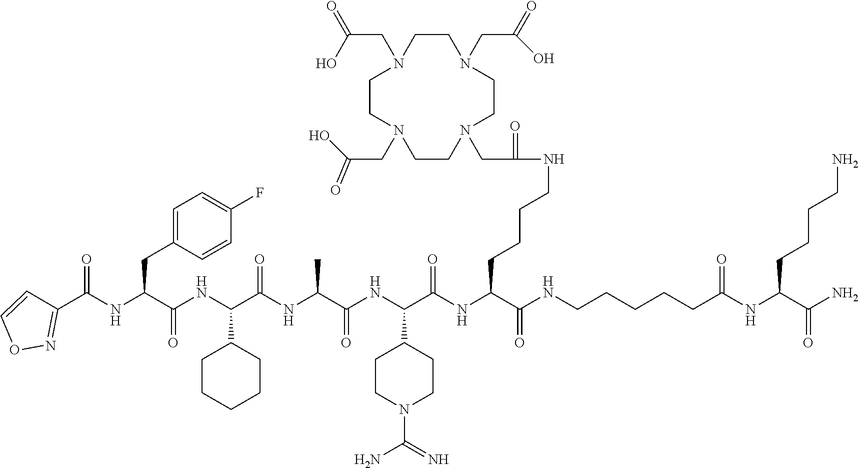 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₆₆H₁₀₅FN₁₈O₁₆: 1424.79, found [M+2H]²⁺: 713.8.

### Example 402: (Compound 402) (SEQ ID NO: 555)

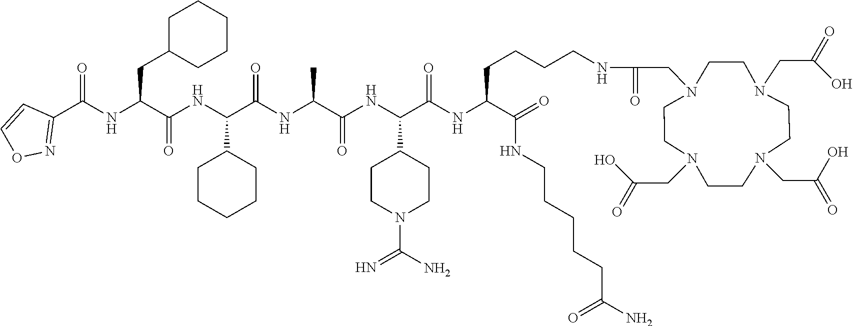 [SMILES: C[C@H](NC(=O)[C@@H](NC(=O)[C@H](CC1CCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₀H₁₀₀N₁₆O₁₅: 1284.76, found [M+2H]²⁺: 643.8.

### Synthesis of Compound 402-In (SEQ ID NO: 697)

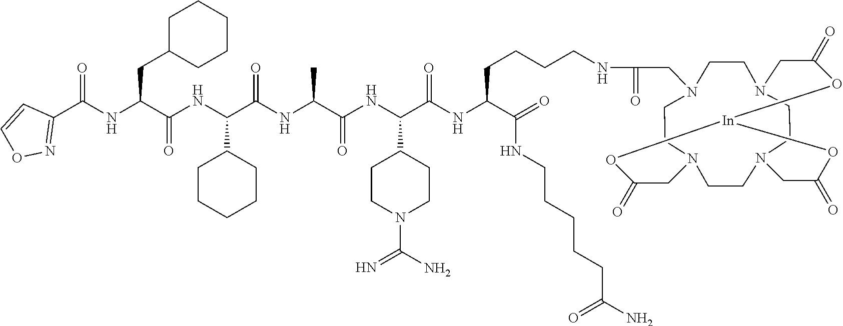

MS: Calc'd for C₆₆H₉₇InN₁₆O₁₅: 1396.64, found [M+2H]²⁺: 699.6.

### Example 403: (Compound 403) (SEQ ID NO: 556)

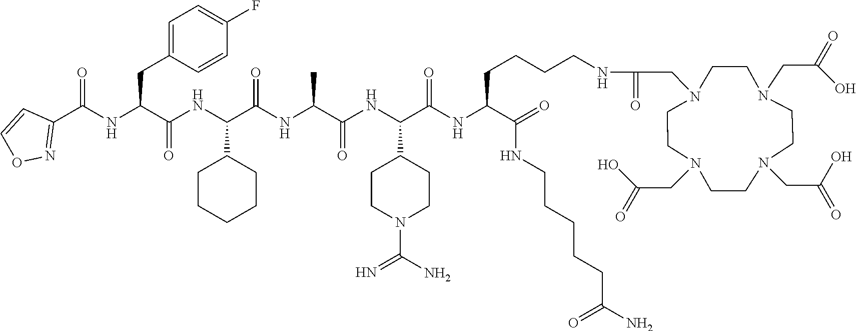 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₀H₉₃FN₁₆O₁₅: 1296.70, found [M+2H]²⁺: 649.8.

### Example 404: (Compound 404) (SEQ ID NO: 557)

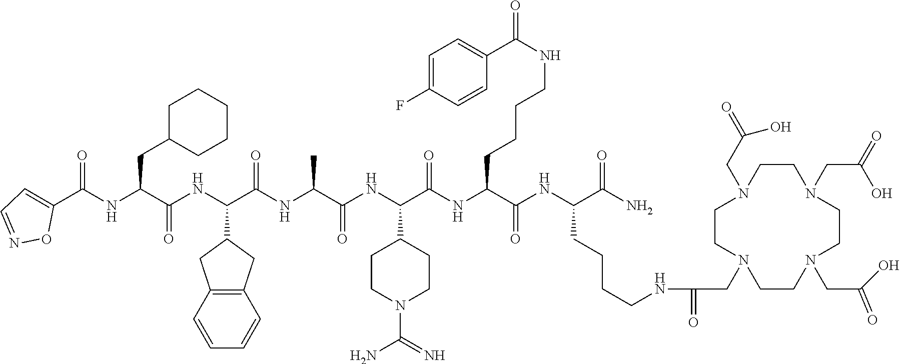 [SMILES: C[C@H](NC(=O)[C@@H](NC(=O)[C@H](CC1CCCCC1)NC(=O)c1ccno1)C1Cc2ccccc2C1)C(=O)N[C@H](C(=O)N[C@@H](CCCCNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₇₀H₁₀₂FN 17016:1455.77, found [M+2H]²⁺: 729.35.

### Example 405: (Compound 405) (SEQ ID NO: 558)

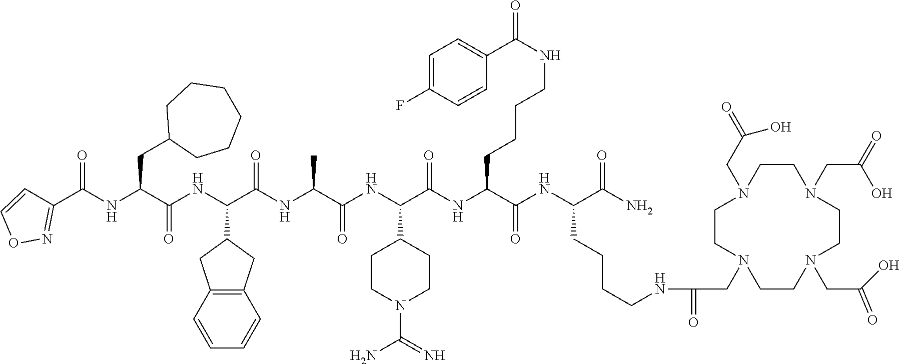 [SMILES: C[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₇₁H₁₀₄FN₁₇O₁₆: 1469.78, found [M+2H]²⁺: 736.4.

### Example 406: (Compound 406) (SEQ ID NO: 559)

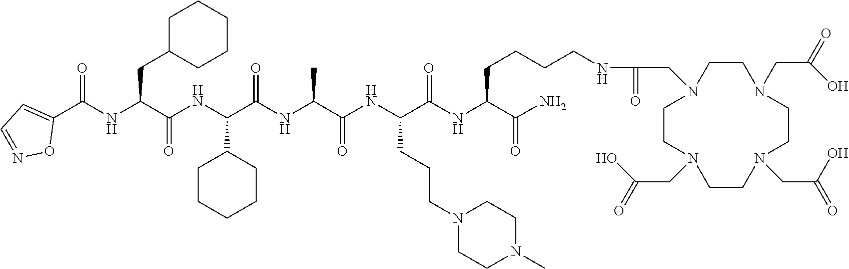 [SMILES: C[C@H](NC(=O)[C@@H](NC(=O)[C@H](CC1CCCCC1)NC(=O)c1ccno1)C1CCCCC1)C(=O)N[C@@H](CCCN1CCN(C)CC1)C(=O)N[C@@H](CCCCNC(=O)CN1CCN(CC(=O)O)CCN(CC(=O)O)CCN(CC(=O)O)CC1)C(N)=O]

### Example 407: (Compound 407) (SEQ ID NO: 560)

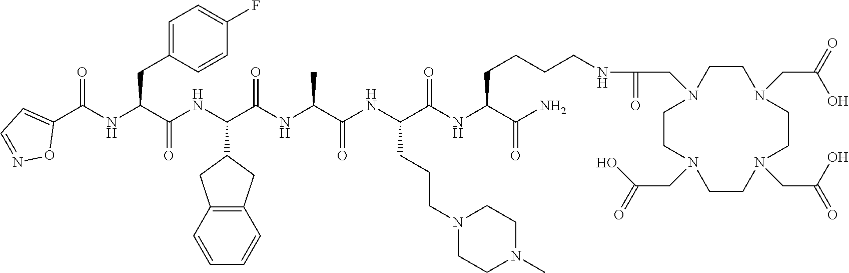 [SMILES: C[C@H](NC(=O)[C@@H](NC(=O)[C@H](Cc1ccc(F)cc1)NC(=O)c1ccno1)C1Cc2ccccc2C1)C(=O)N[C@@H](CCCN1CCN(C)CC1)C(=O)N[C@@H](CCCCNC(=O)CN1CCN(CC(=O)O)CCN(CC(=O)O)CCN(CC(=O)O)CC1)C(N)=O]

### Example 408: (Compound 408) (SEQ ID NO: 561)

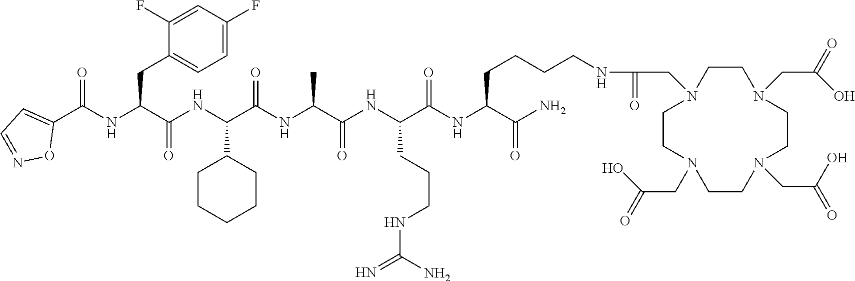 [SMILES: C[C@H](NC(=O)[C@@H](NC(=O)[C@H](Cc1ccc(F)cc1F)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₇₉F₂N₁₅O₁₄: 1175.59, found [M+2H]²⁺: 589.2.

### Example 409: (Compound 409) (SEQ ID NO: 562)

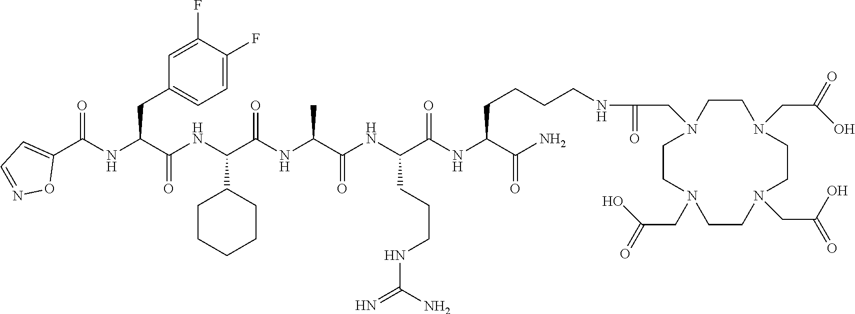 [SMILES: C[C@H](NC(=O)[C@@H](NC(=O)[C@H](Cc1ccc(F)c(F)c1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₇₉F₂N₁₅O₁₄: 1175.59, found [M+2H]²⁺: 589.2.

### Example 410: (Compound 410) (SEQ ID NO: 563)

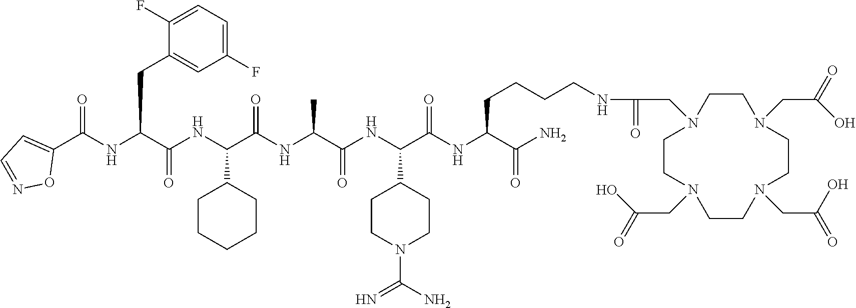 [SMILES: C[C@H](NC(=O)[C@@H](NC(=O)[C@H](Cc1cc(F)ccc1F)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₄H₈₁F₂N₁₅O₁₄: 1201.61, found [M+2H]²⁺: 601.9.

### Example 411: (Compound 411) (SEQ ID NO: 564)

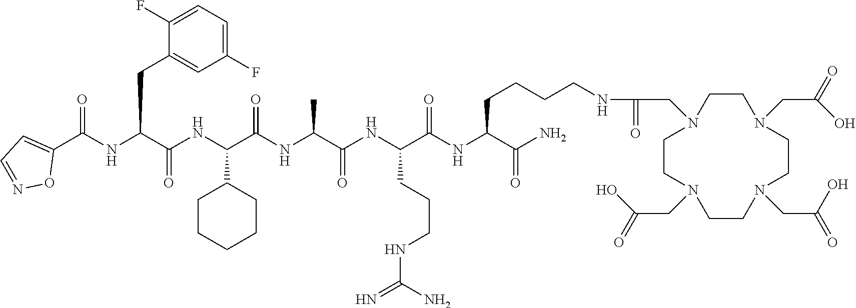 [SMILES: C[C@H](NC(=O)[C@@H](NC(=O)[C@H](Cc1cc(F)ccc1F)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₇₉F₂N₁₅O₁₄: 1175.59, found [M+2H]²⁺: 588.9.

### Example 412: (Compound 412)

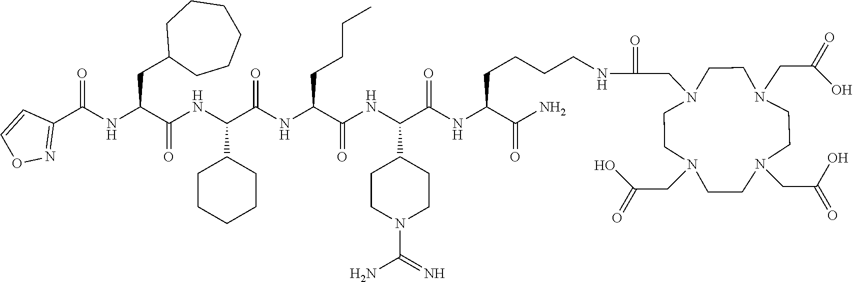 [SMILES: CCCC[C@H](NC(=O)[C@@H](NC(=O)[C@H](CC1CCCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₈H₉₇N₁₅O₁₄: 1227.73, found [M+2H]²⁺: 615.3.

### Example 413: (Compound 413) (SEQ ID NO: 566)

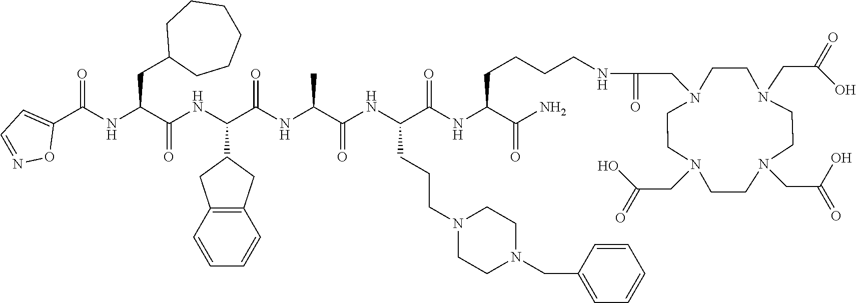 [SMILES: C[C@H](NC(=O)[C@@H](NC(=O)[C@H](CC1CCCCCC1)NC(=O)c1ccno1)C1Cc2ccccc2C1)C(=O)N[C@@H](CCCN1CCN(Cc2ccccc2)CC1)C(=O)N[C@@H](CCCCNC(=O)CN1CCN(CC(=O)O)CCN(CC(=O)O)CCN(CC(=O)O)CC1)C(N)=O]

### Example 414: (Compound 414) (SEQ ID NO: 567)

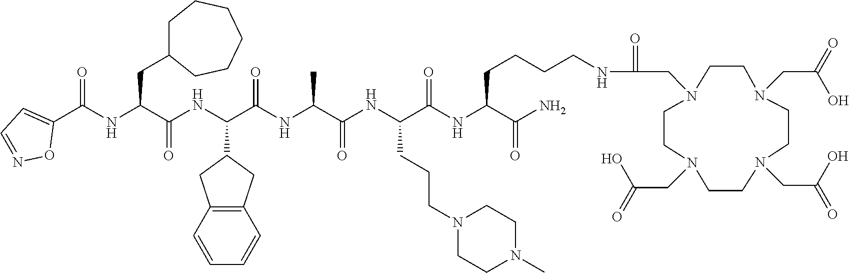 [SMILES: C[C@H](NC(=O)[C@@H](NC(=O)[C@H](CC1CCCCCC1)NC(=O)c1ccno1)C1Cc2ccccc2C1)C(=O)N[C@@H](CCCN1CCN(C)CC1)C(=O)N[C@@H](CCCCNC(=O)CN1CCN(CC(=O)O)CCN(CC(=O)O)CCN(CC(=O)O)CC1)C(N)=O]

### Example 415: (Compound 415) (SEQ ID NO: 568)

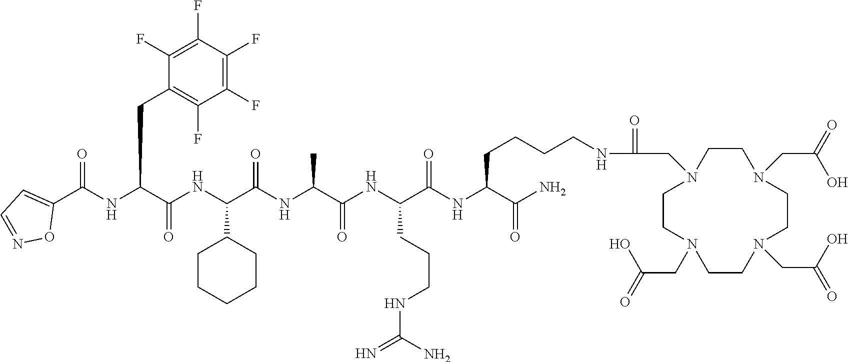 [SMILES: C[C@H](NC(=O)[C@@H](NC(=O)[C@H](Cc1c(F)c(F)c(F)c(F)c1F)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₇₆F₅N₁₅O₁₄: 1229.56, found [M+2H]²⁺: 615.9.

### Example 416: (Compound 416) (SEQ ID NO: 569)

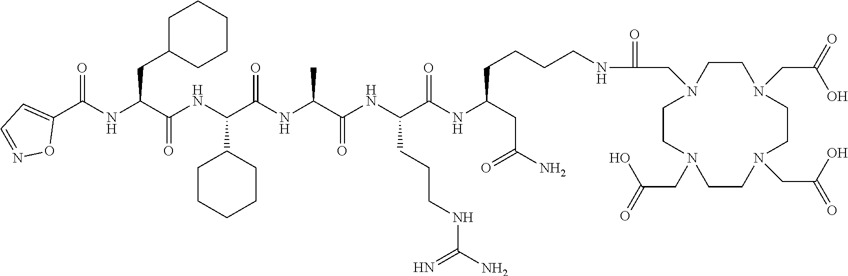 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)CC(N)=O]

MS: Calc'd for C₅₃H₈₉N₁₅O₁₄: 1159.67, found [M+2H]²⁺: 581.1.

### Example 417: (Compound 417)

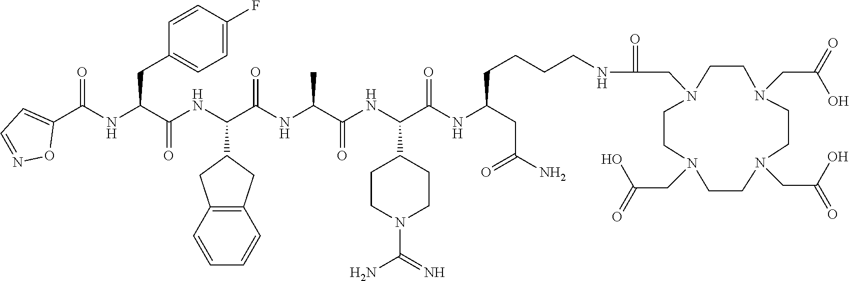 [SMILES: C[C@H](NC(=O)[C@@H](NC(=O)[C@H](Cc1ccc(F)cc1)NC(=O)c1ccno1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)CC(N)=O)C1CCN(C(=N)N)CC1]

MS: Calc'd for C₅₈H₈₂FN₁₅O₁₄: 1231.62, found [M+2H]²⁺: 617.05.

### Example 418: (Compound 418)

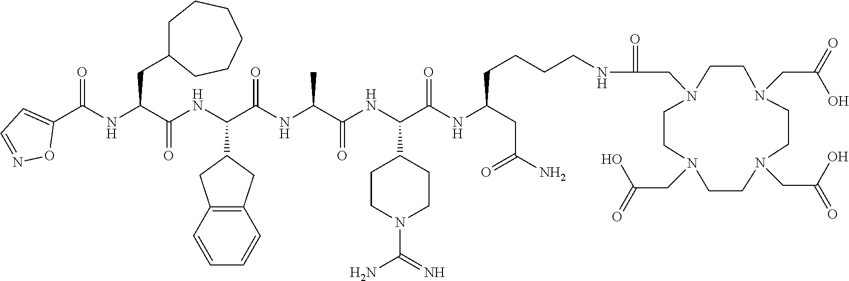 [SMILES: C[C@H](NC(=O)[C@@H](NC(=O)[C@H](CC1CCCCCC1)NC(=O)c1ccno1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)CC(N)=O)C1CCN(C(=N)N)CC1]

MS: Calc'd for C₅₉H₉₁N₁₅O₁₄: 1233.69, found [M+2H]²⁺: 618.1.

### Example 419: (Compound 419)

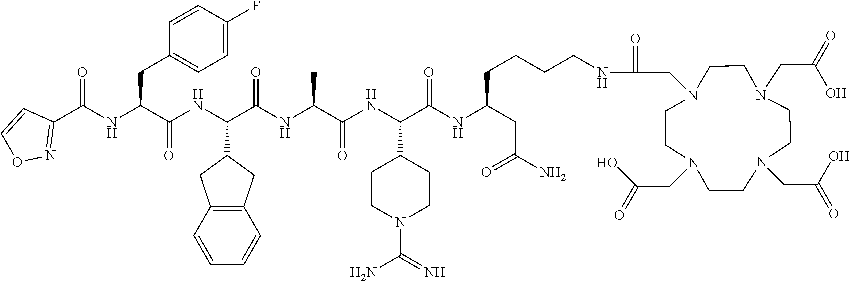 [SMILES: C[C@H](NC(=O)[C@@H](NC(=O)[C@H](Cc1ccc(F)cc1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)CC(N)=O)C1CCN(C(=N)N)CC1]

MS: Calc'd for C₅₈H₈₂FN₁₅O₁₄: 1231.62, found [M+2H]²⁺: 617.05.

### Example 420: (Compound 420)

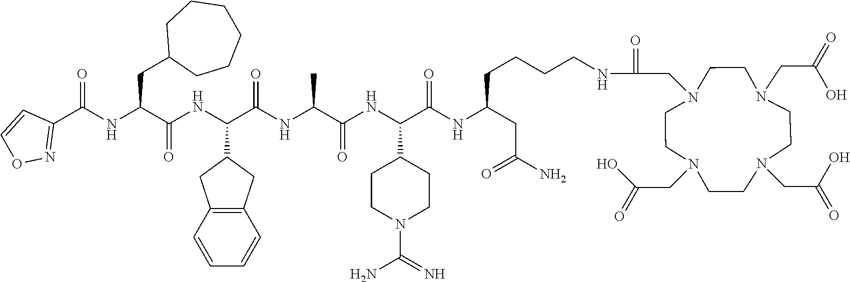 [SMILES: C[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)CC(N)=O)C1CCN(C(=N)N)CC1]

MS: Calc'd for C₅₉H₉₁N₁₅O₁₄: 1233.69, found [M+2H]²⁺: 618.1.

### Example 421: (Compound 421) (SEQ ID NO: 574)

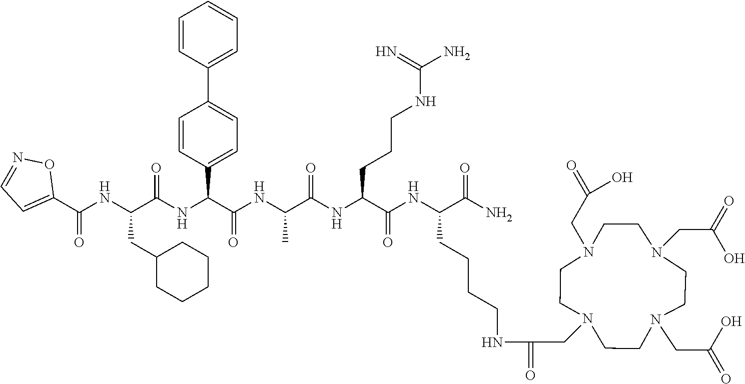 [SMILES: C[C@H](NC(=O)[C@@H](NC(=O)[C@H](CC1CCCCC1)NC(=O)c1ccno1)c1ccc(-c2ccccc2)cc1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₈H₈₅N₁₅O₁₄: 1215.64, found [M+2H]²⁺: 608.9.

### Synthesis of Compound 421-In (SEQ ID NO: 698)

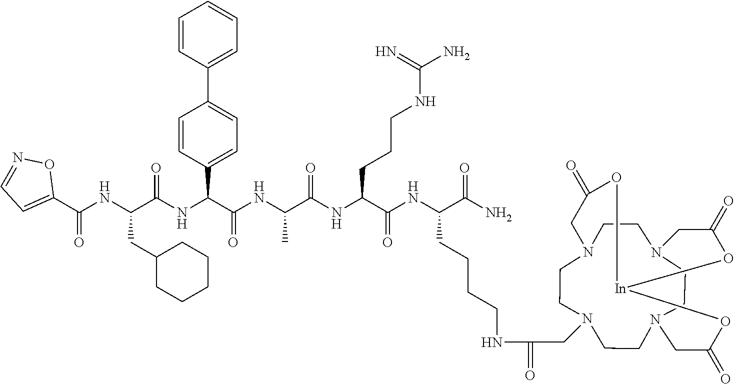

MS: Calc'd for C₅₈H₈₅N₁₅O₁₄: 1215.64, found [M+2H]²⁺: 608.9.

### Example 422: (Compound 422) (SEQ ID NO: 575)

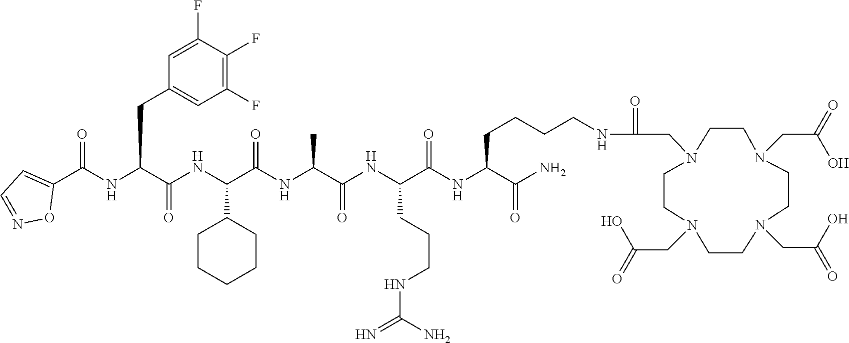 [SMILES: C[C@H](NC(=O)[C@@H](NC(=O)[C@H](Cc1cc(F)c(F)c(F)c1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₇₈F₃N₁₅O₁₄: 1193.58, found [M+2H]²⁺: 598.0.

### Example 423: (Compound 423) (SEQ ID NO: 576)

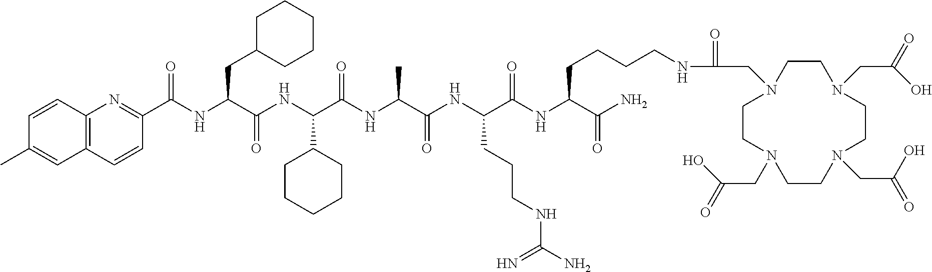 [SMILES: Cc1ccc2nc(C(=O)N[C@@H](CC3CCCCC3)C(=O)N[C@H](C(=O)N[C@@H](C)C(=O)N[C@@H](CCCNC(=N)N)C(=O)N[C@@H](CCCCNC(=O)CN3CCN(CC(=O)O)CCN(CC(=O)O)CCN(CC(=O)O)CC3)C(N)=O)C3CCCCC3)ccc2c1]

MS: Calc'd for C₅₉H₉₃N₁₅O₁₃: 1219.71, found [M+2H]²⁺: 611.0.

### Example 424: (Compound 424) (SEQ ID NO: 577)

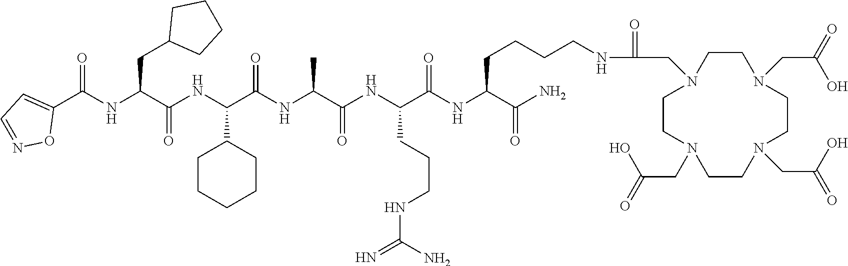 [SMILES: C[C@H](NC(=O)[C@@H](NC(=O)[C@H](CC1CCCC1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₁H₈₅N₁₅O₁₄: 1131.64, found [M+2H]²⁺: 567.0.

### Example 425: (Compound 425) (SEQ ID NO: 578)

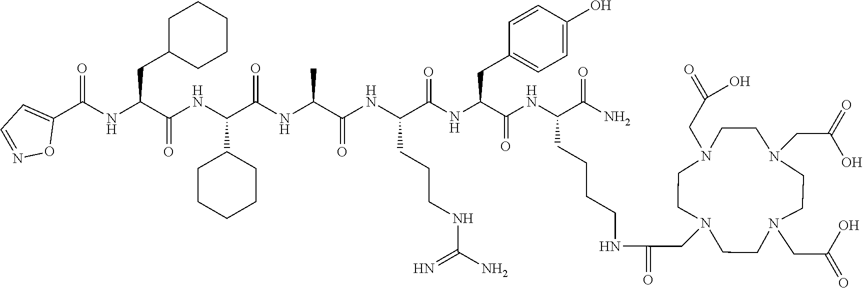 [SMILES: C[C@H](NC(=O)[C@@H](NC(=O)[C@H](CC1CCCCC1)NC(=O)c1ccno1)C1CCCCC1)C(=O)N[C@@H](CCCNC(=N)N)C(=O)N[C@@H](Cc1ccc(O)cc1)C(=O)N[C@@H](CCCCNC(=O)CN1CCN(CC(=O)O)CCN(CC(=O)O)CCN(CC(=O)O)CC1)C(N)=O]

MS: Calc'd for C₆₁H₉₆N₁₆O₁₆: 1308.72, found [M+2H]²⁺: 655.5.

### Example 426: (Compound 426) (SEQ ID NO: 579)

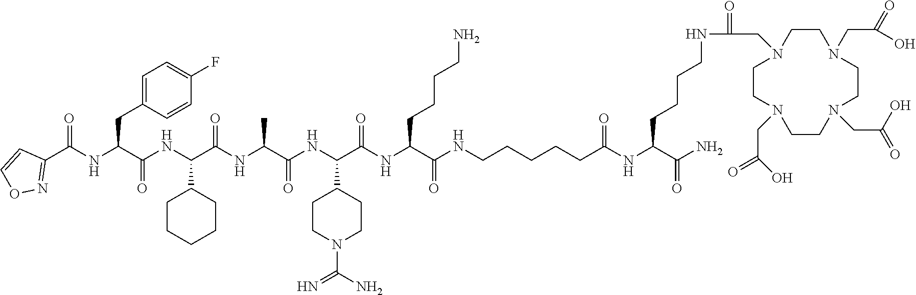 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCN)C(=O)NCCCCCC(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₆H₁₀₅FN₁₈O₁₆: 1424.79, found [M+3H]⁺: 476.3.

### Example 427: (Compound 427) (SEQ ID NO: 580)

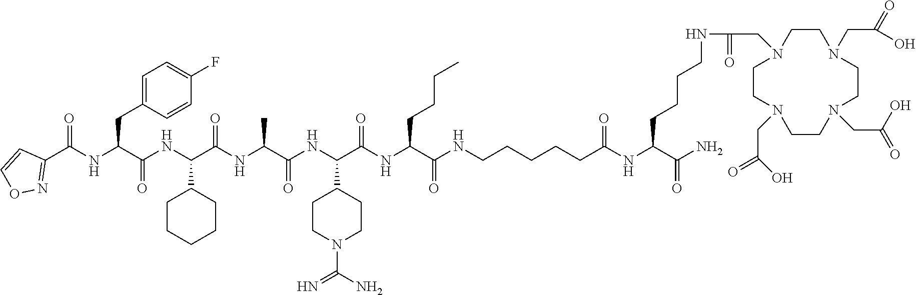 [SMILES: CCCC[C@H](NC(=O)[C@@H](NC(=O)[C@H](C)NC(=O)[C@@H](NC(=O)[C@H](Cc1ccc(F)cc1)NC(=O)c1ccon1)C1CCCCC1)C1CCN(C(=N)N)CC1)C(=O)NCCCCCC(=O)N[C@@H](CCCCNC(=O)CN1CCN(CC(=O)O)CCN(CC(=O)O)CCN(CC(=O)O)CC1)C(N)=O]

MS: Calc'd for C₆₆H₁₀₄FN₁₇O₁₆: 1409.78, found [M+3H]⁺: 471.4.

### Example 428: (Compound 428) (SEQ ID NO: 581)

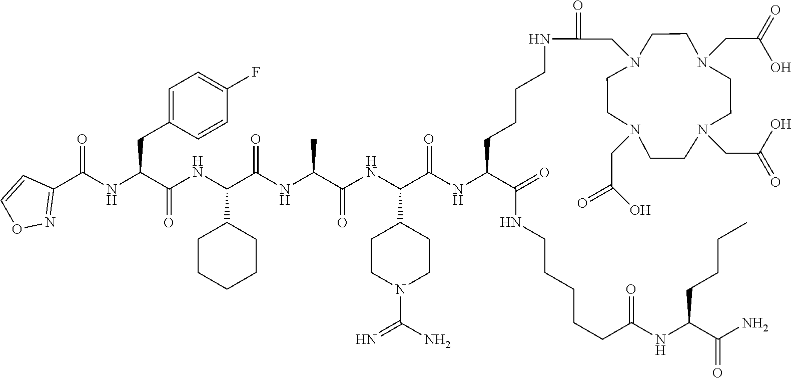 [SMILES: CCCC[C@H](NC(=O)CCCCCNC(=O)[C@H](CCCCNC(=O)CN1CCN(CC(=O)O)CCN(CC(=O)O)CCN(CC(=O)O)CC1)NC(=O)[C@@H](NC(=O)[C@H](C)NC(=O)[C@@H](NC(=O)[C@H](Cc1ccc(F)cc1)NC(=O)c1ccon1)C1CCCCC1)C1CCN(C(=N)N)CC1)C(N)=O]

MS: Calc'd for C₆₆H₁₀₄FN₁₇O₁₆: 1409.78, found [M+2H]²⁺: 706.35.

### Example 429: (Compound 429) (SEQ ID NO: 582)

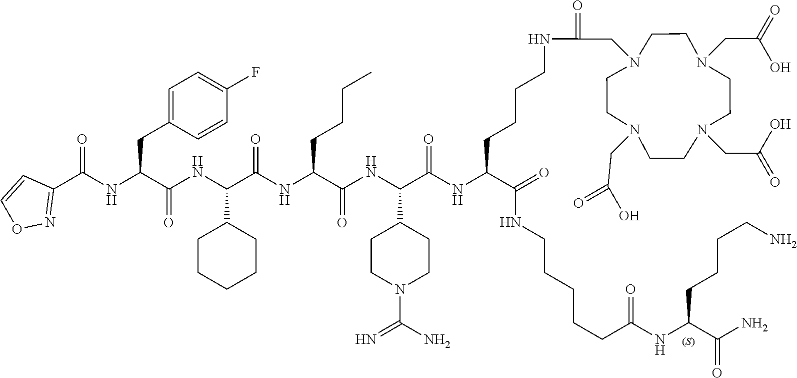

MS: Calc'd for C₆₉H₁₁₁FN₁₈O₁₆: 1466.84, found [M+3H]³⁺: 490.35.

### Example 430: (Compound 430) (SEQ ID NO: 583)

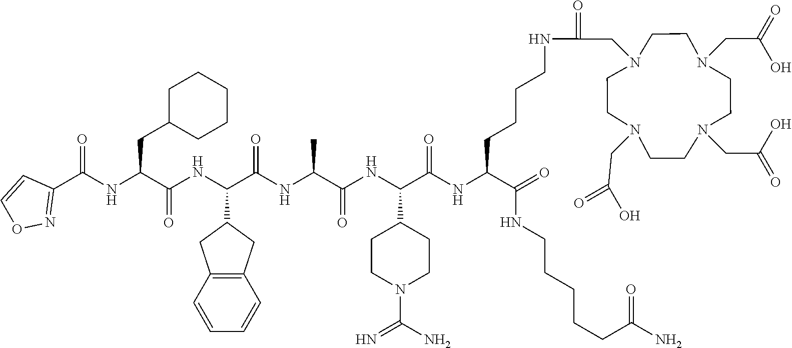 [SMILES: C[C@H](NC(=O)[C@@H](NC(=O)[C@H](CC1CCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₃H₉₈N₁₆O₁₅: 1318.74, found [M+2H]²⁺: 660.8.

### Example 431: (Compound 431) (SEQ ID NO: 584)

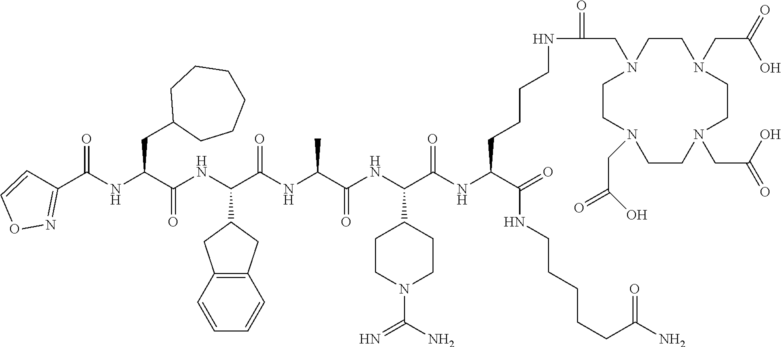 [SMILES: C[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₄H₁₀₀N₁₆O₁₅: 1332.76, found [M+2H]²⁺: 667.8.

### Example 432: (Compound 432)

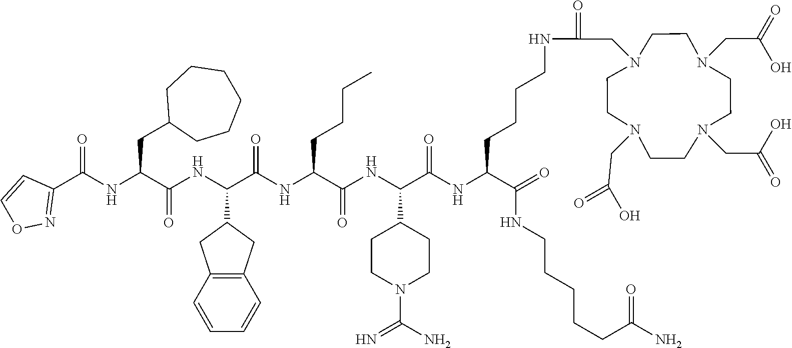 [SMILES: CCCC[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₇H₁₀₆N₁₆O₁₅: 1374.80, found [M+2H]²⁺: 688.85.

### Example 433: (Compound 433) (SEQ ID NO: 586)

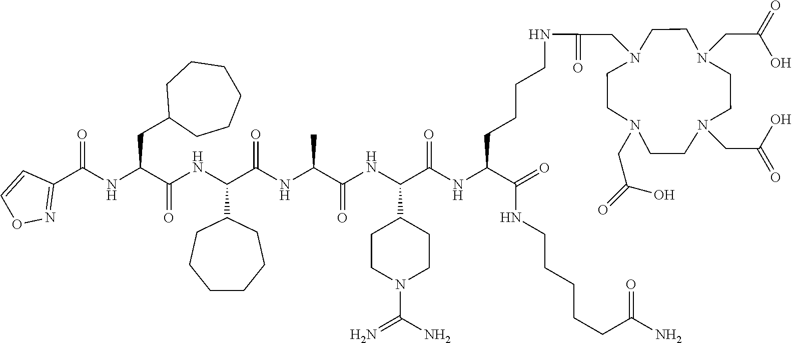 [SMILES: C[C@H](NC(=O)[C@@H](NC(=O)[C@H](CC1CCCCCC1)NC(=O)c1ccon1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₂H₁₀₄N₁₆O₁₅: 1312.79, found [M+3H]⁺: 439.0.

### Example 434: (Compound 434) (SEQ ID NO: 587)

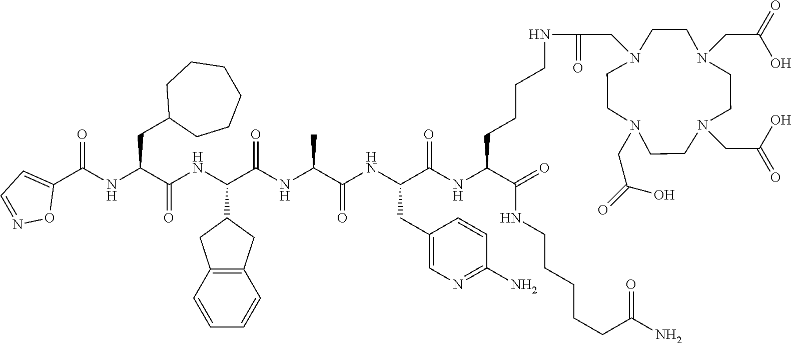 [SMILES: C[C@H](NC(=O)[C@@H](NC(=O)[C@H](CC1CCCCCC1)NC(=O)c1ccno1)C1Cc2ccccc2C1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(=O)NCCCCCC(N)=O]

MS: Calc'd for C₆₄H₉₅N₁₅O₁₅: 1313.71, found [M+2H]²⁺: 657.9.

### Example 435: (Compound 435) (SEQ ID NO: 588)

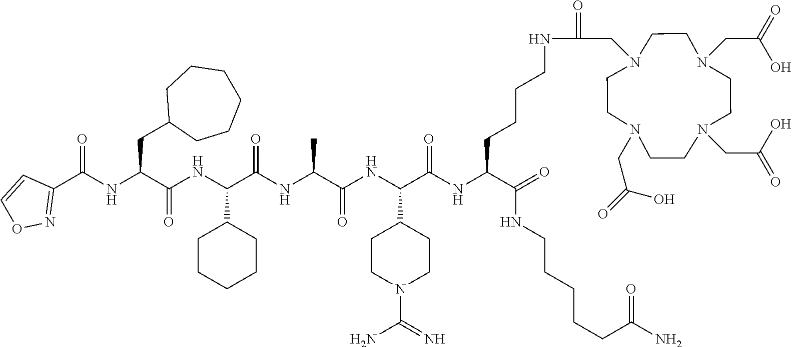 [SMILES: C[C@H](NC(=O)[C@@H](NC(=O)[C@H](CC1CCCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1]

MS: Calc'd for C₆₁H₁₀₂N₁₆O₁₅: 1298.77, found [M+3H]⁺: 434.25.

### Example 436: (Compound 436) (SEQ ID NO: 589)

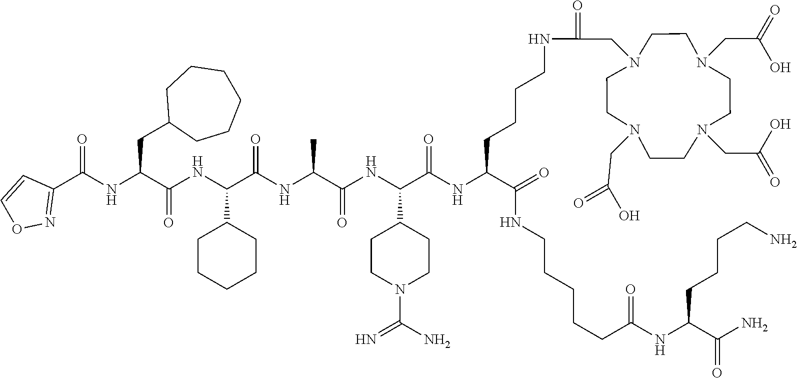 [SMILES: C[C@H](NC(=O)[C@@H](NC(=O)[C@H](CC1CCCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₆₇H₁₁₄N₁₈O₁₆: 1426.87, found [M+3H]⁺: 477.0.

### Example 437: (Compound 437) (SEQ ID NO: 590)

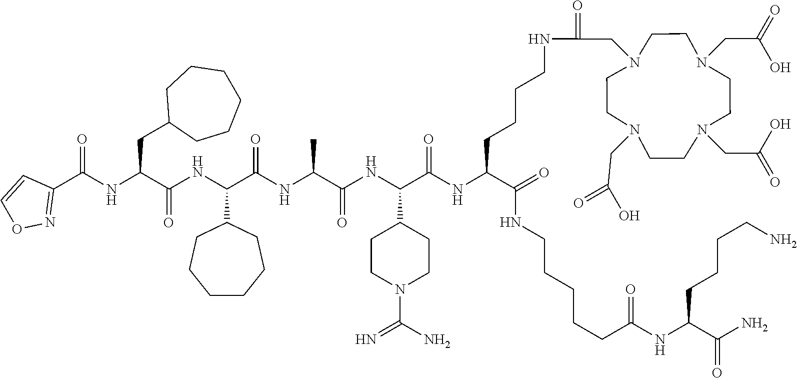 [SMILES: C[C@H](NC(=O)[C@@H](NC(=O)[C@H](CC1CCCCCC1)NC(=O)c1ccon1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₆₈H₁₁₆N₁₈O₁₆: 1440.88, found [M+3H]⁺: 481.65.

### Example 438: (Compound 438) (SEQ ID NO: 591)

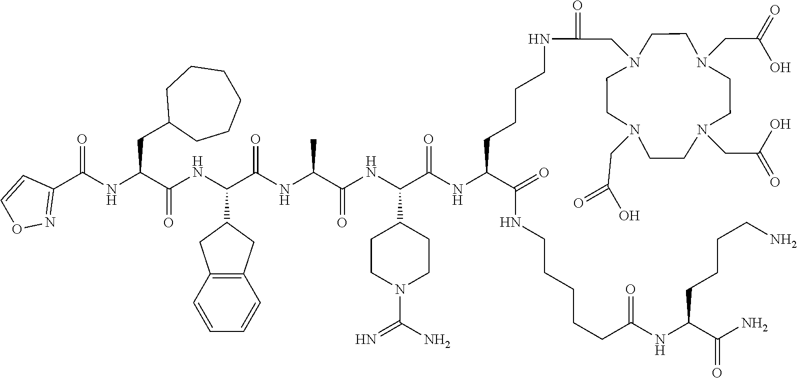 [SMILES: C[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₇₀H₁₁₂N₁₈O₁₆: 1460.85, found [M+3H]⁺: 488.35.

### Example 439: (Compound 439) (SEQ ID NO: 592)

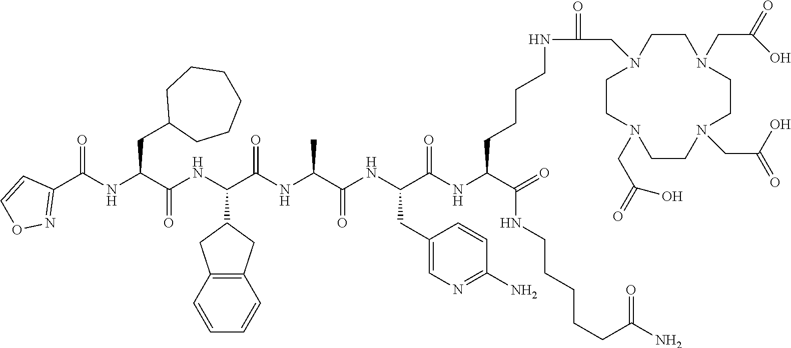 [SMILES: C[C@H](NC(=O)[C@@H](NC(=O)[C@H](CC1CCCCCC1)NC(=O)c1ccon1)C1Cc2ccccc2C1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(=O)NCCCCCC(N)=O]

MS: Calc'd for C₆₄H₉₅N₁₅O₁₅: 1313.71, found [M+2H]²⁺: 658.0.

### Example 440: (Compound 440) (SEQ ID NO: 593)

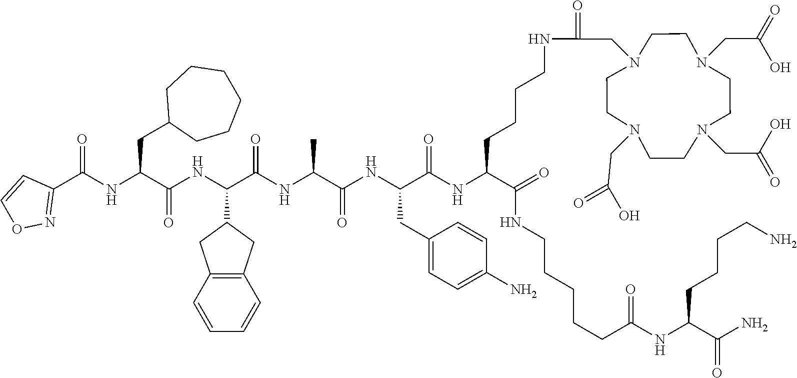 [SMILES: C[C@H](NC(=O)[C@@H](NC(=O)[C@H](CC1CCCCCC1)NC(=O)c1ccon1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O]

MS: Calc'd for C₇₁H₁₀₈N₁₆O₁₆: 1440.81, found [M+H]⁺: 1441.4.

### Example 441: (Compound 441) (SEQ ID NO: 594)

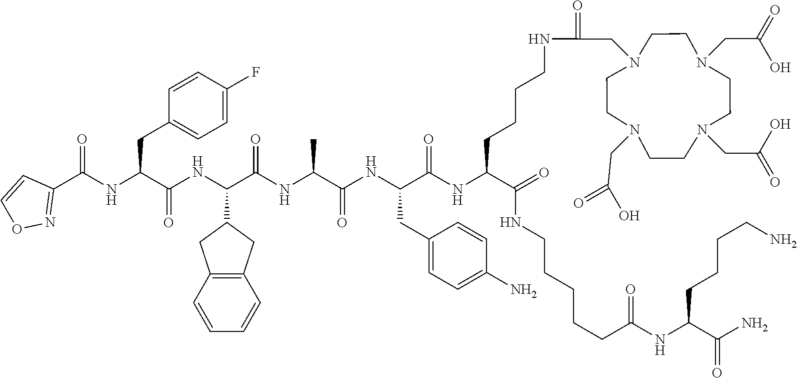 [SMILES: C[C@H](NC(=O)[C@@H](NC(=O)[C@H](Cc1ccc(F)cc1)NC(=O)c1ccon1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O]

MS: Calc'd for C₇₀H₉₉FN₁₆O₁₆: 1438.74, found [M+H]⁺: 1439.25.

### Example 442: (Compound 442) (SEQ ID NO: 595)

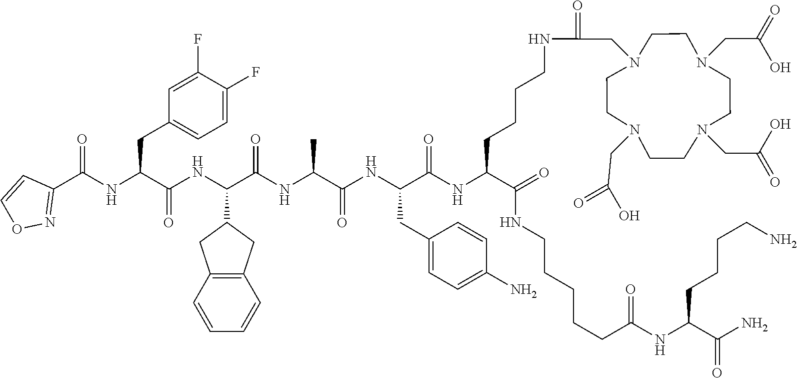 [SMILES: C[C@H](NC(=O)[C@@H](NC(=O)[C@H](Cc1ccc(F)c(F)c1)NC(=O)c1ccon1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O]

MS: Calc'd for C₇₀H₉₈F₂N₁₆O₁₆: 1456.73, found [M+3H]³⁺: 487.0.

### Example 443: (Compound 443) (SEQ ID NO: 596)

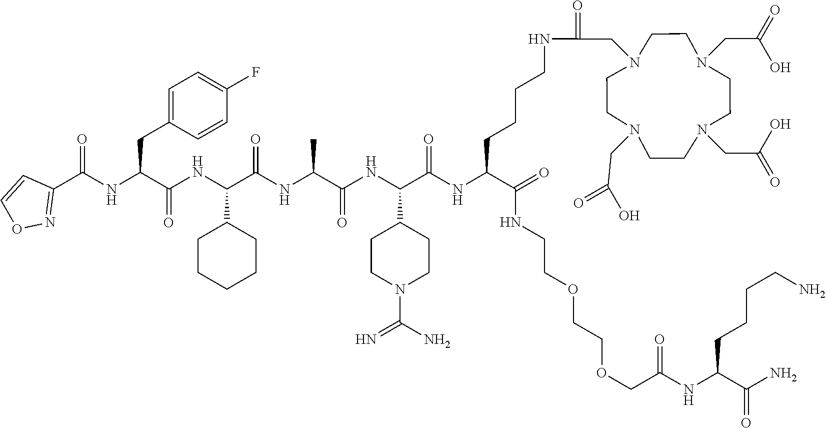 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCN(C(=N)N)CC1]

MS: Calc'd for C₆₆H₁₀₅FN₁₈O₁₈: 1456.78, found [M+3H]³⁺: 486.8.

### Example 444: (Compound 444) (SEQ ID NO: 597)

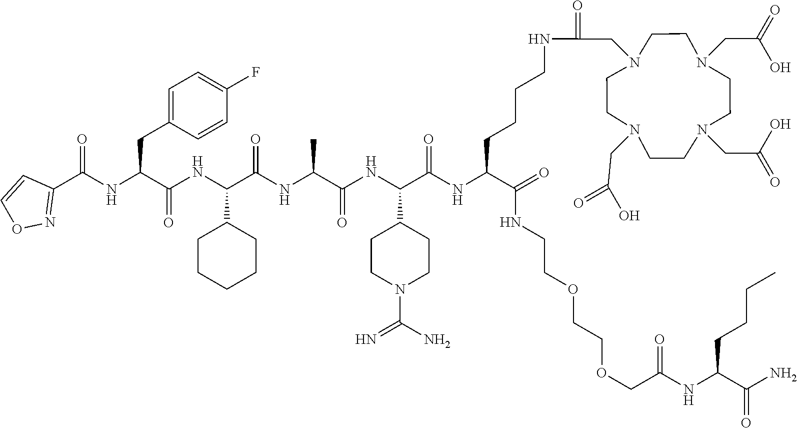 [SMILES: CCCC[C@H](NC(=O)COCCOCCNC(=O)[C@H](CCCCNC(=O)CN1CCN(CC(=O)O)CCN(CC(=O)O)CCN(CC(=O)O)CC1)NC(=O)[C@@H](NC(=O)[C@H](C)NC(=O)[C@@H](NC(=O)[C@H](Cc1ccc(F)cc1)NC(=O)c1ccon1)C1CCCCC1)C1CCN(C(=N)N)CC1)C(N)=O]

MS: Calc'd for C₆₆H₁₀₄FN₁₇O₁₈: 1441.77, found [M+2H]²⁺: 722.4.

### Example 445: (Compound 445) (SEQ ID NO: 598)

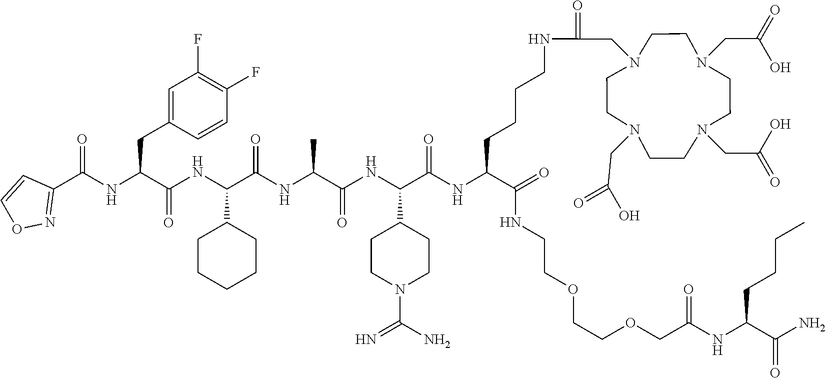 [SMILES: CCCC[C@H](NC(=O)COCCOCCNC(=O)[C@H](CCCCNC(=O)CN1CCN(CC(=O)O)CCN(CC(=O)O)CCN(CC(=O)O)CC1)NC(=O)[C@@H](NC(=O)[C@H](C)NC(=O)[C@@H](NC(=O)[C@H](Cc1ccc(F)c(F)c1)NC(=O)c1ccon1)C1CCCCC1)C1CCN(C(=N)N)CC1)C(N)=O]

MS: Calc'd for C₆₆H₁₀₃F₂N₁₇O₁₈: 1459.76, found [M+3H]³⁺: 487.9.

### Example 446: (Compound 446) (SEQ ID NO: 599)

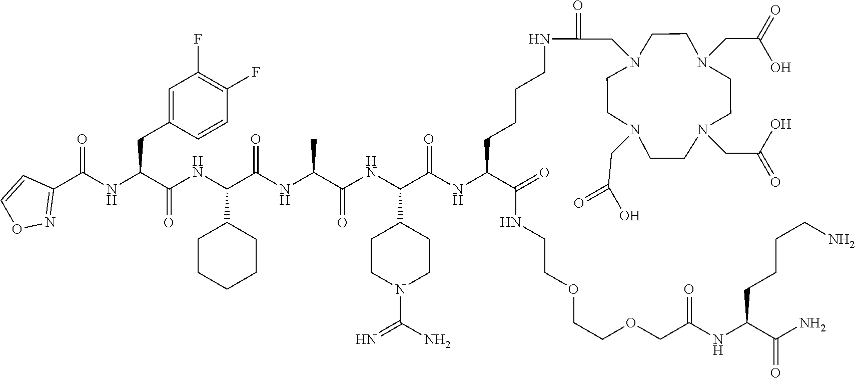 [SMILES: C[C@H](NC(=O)[C@@H](NC(=O)[C@H](Cc1ccc(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCN(C(=N)N)CC1]

MS: Calc'd for C₆₆H₁₀₄F₂N₁₈O₁₈: 1474.77, found [M+3H]³⁺: 493.0.

### Example 447: (Compound 447) (SEQ ID NO: 600)

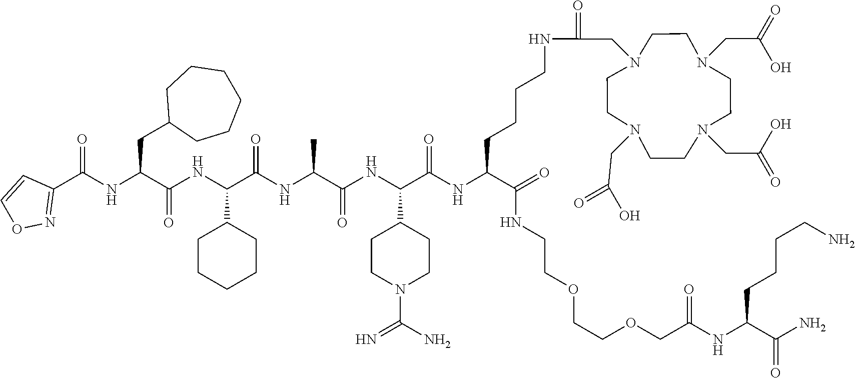 [SMILES: C[C@H](NC(=O)[C@@H](NC(=O)[C@H](CC1CCCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCN(C(=N)N)CC1]

MS: Calc'd for C₆₇H₁₁₄N₁₈O₁₈: 1458.86, found [M+3H]⁺: 487.7.

### Example 448: (Compound 448) (SEQ ID NO: 601)

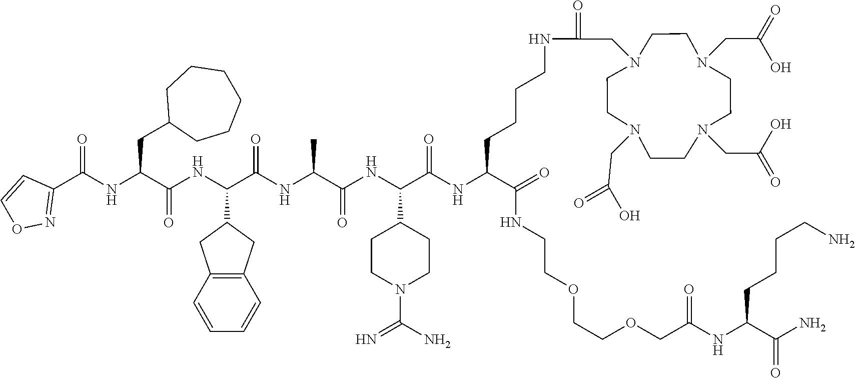 [SMILES: C[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCN(C(=N)N)CC1]

MS: Calc'd for C₇₀H₁₁₂N₁₈O₁₈: 1492.84, found [M+3H]³⁺: 498.9.

### Example 449: (Compound 449) (SEQ ID NO: 602)

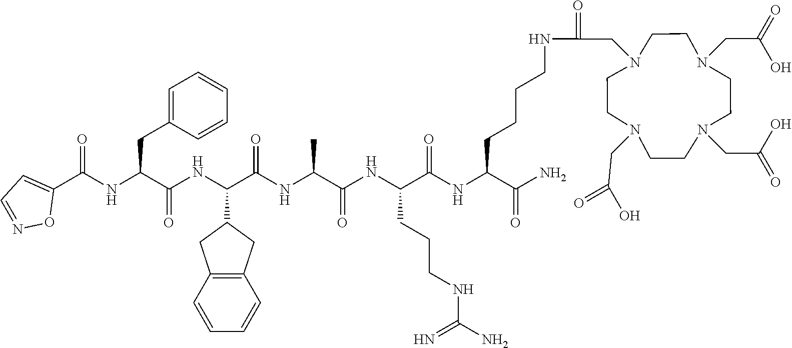 [SMILES: C[C@H](NC(=O)[C@@H](NC(=O)[C@H](Cc1ccccc1)NC(=O)c1ccno1)C1Cc2ccccc2C1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₅H₇₉N₁₅O₁₄: 1173.59, found [M+2H]²⁺: 588.2.

### Example 450: (Compound 450) (SEQ ID NO: 603)

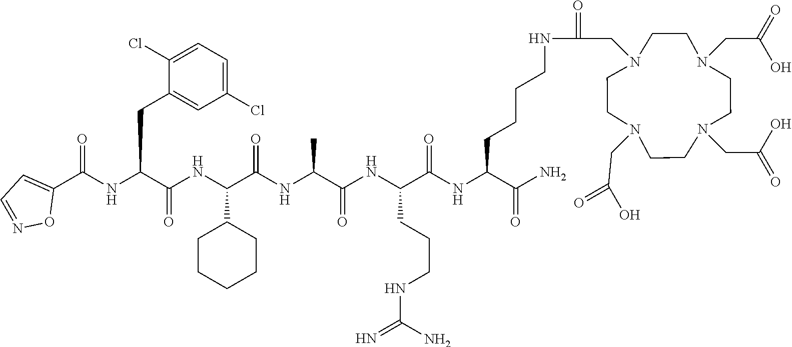 [SMILES: C[C@H](NC(=O)[C@@H](NC(=O)[C@H](Cc1cc(Cl)ccc1Cl)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

MS: Calc'd for C₅₂H₇₉Cl₂N₁₅O₁₄: 1207.53, found [M+3H]⁺: 404.2.

### Example 451: (Compound 451) (SEQ ID NO: 604)

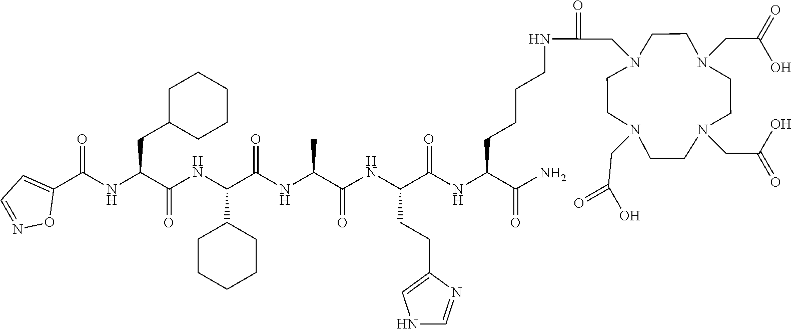 [SMILES: C[C@H](NC(=O)[C@@H](NC(=O)[C@H](CC1CCCCC1)NC(=O)c1ccno1)C1CCCCC1)C(=O)N[C@@H](CCc1c[nH]cn1)C(=O)N[C@@H](CCCCNC(=O)CN1CCN(CC(=O)O)CCN(CC(=O)O)CCN(CC(=O)O)CC1)C(N)=O]

### Example 452: (Compound 452) (SEQ ID NO: 605)

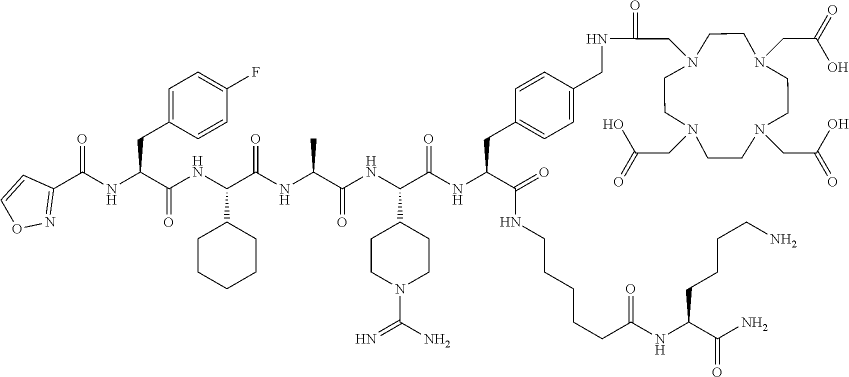 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](Cc1ccc(CNC(=O)CN2CCN(CC(=O)O)CCN(CC(=O)O)CCN(CC(=O)O)CC2)cc1)C(=O)NCCCCCC(=O)N[C@@H](CCCCN)C(N)=O)C1CCN(C(=N)N)CC1]

MS: Calc'd for C₇₀H₁₀₄FN₁₇O₁₇: 1473.78, found [M+2H]²⁺: 738.15.

### Example 453: (Compound 453) (SEQ ID NO: 606)

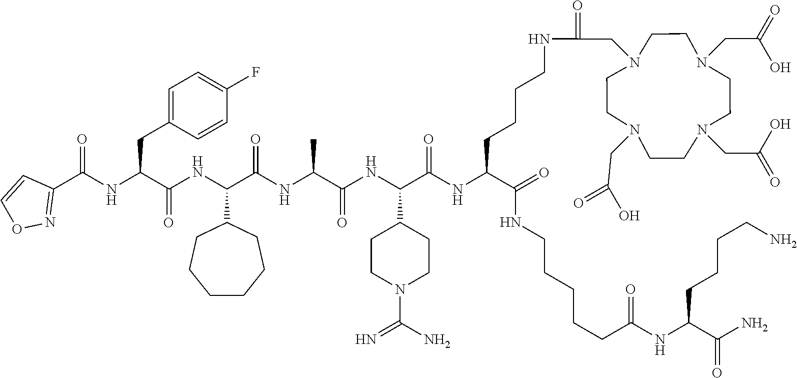 [SMILES: C[C@H](NC(=O)[C@@H](NC(=O)[C@H](Cc1ccc(F)cc1)NC(=O)c1ccon1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₆₇H₁₀₆FN₁₇O₁₇: 1439.79, found [M+2H]²⁺: 721.1.

### Example 454: (Compound 454) (SEQ ID NO: 607)

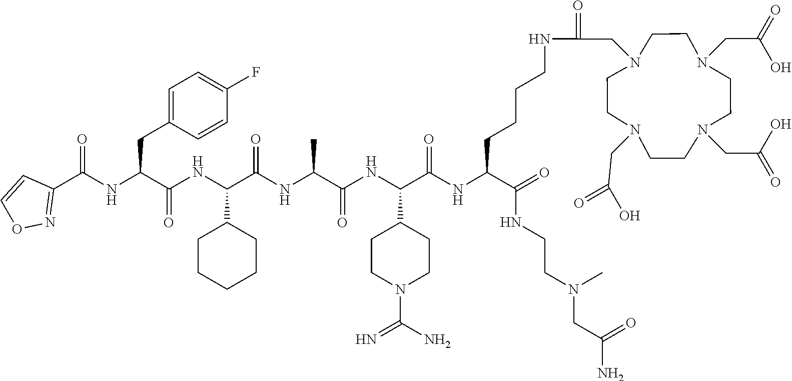 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCN(C)CC(N)=O)C1CCN(C(=N)N)CC1]

MS: Calc'd for C₅₉H₉₂FN₁₇O₁₅: 1298.49, found [M+2H]²⁺: 650.0.

### Example 455: (Compound 455) (SEQ ID NO: 608)

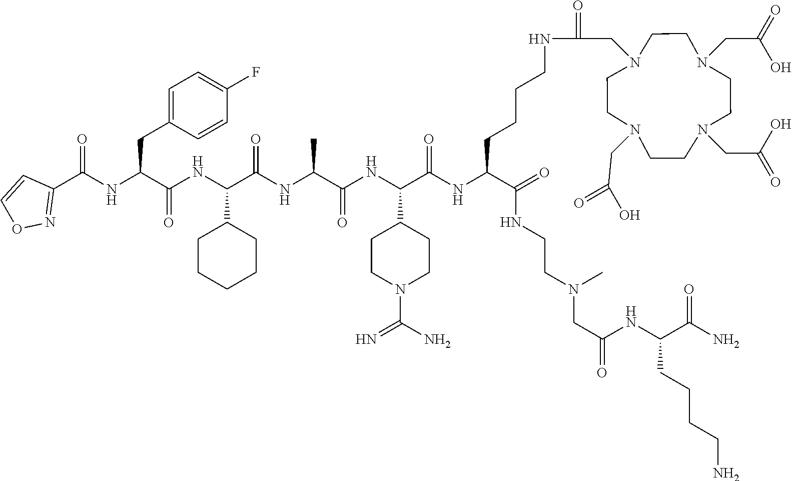 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCN(C)CC(=O)N[C@@H](CCCCN)C(N)=O)C1CCN(C(=N)N)CC1]

MS: Calc'd for C₆₅H₁₀₃FN₁₈O₁₇: 1426.77, found [M+2H]²⁺: 714.6.

### Example 456: (Compound 456) (SEO ID NO: 609)

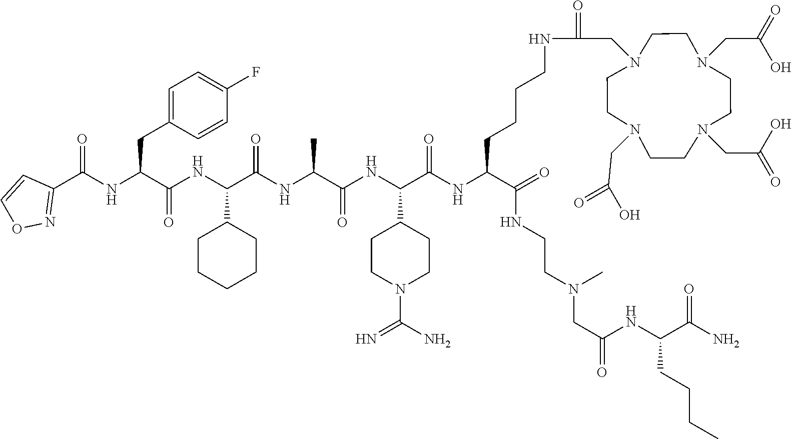 [SMILES: CCCC[C@H](NC(=O)CN(C)CCNC(=O)[C@H](CCCCNC(=O)CN1CCN(CC(=O)O)CCN(CC(=O)O)CCN(CC(=O)O)CC1)NC(=O)[C@@H](NC(=O)[C@H](C)NC(=O)[C@@H](NC(=O)[C@H](Cc1ccc(F)cc1)NC(=O)c1ccon1)C1CCCCC1)C1CCN(C(=N)N)CC1)C(N)=O]

MS: Calc'd for C₆₅H₁₀₃FN₁₈O₁₆: 1410.78, found [M+H]⁺: 1411.2.

### Example 457: (Compound 457) (SEQ ID NO: 610)

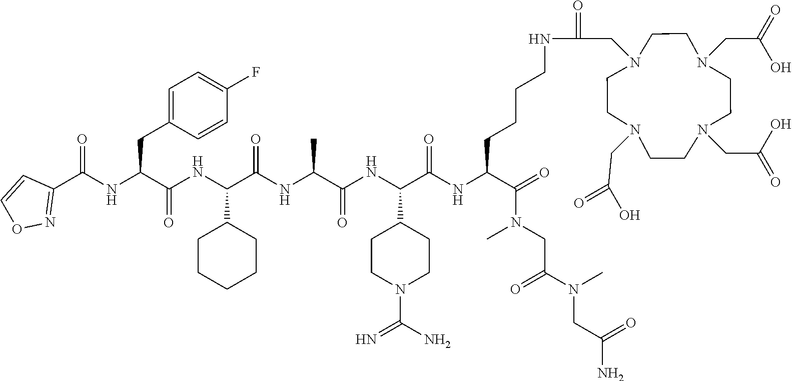 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)N(C)CC(=O)N(C)CC(N)=O)C1CCN(C(=N)N)CC1]

MS: Calc'd for C₆₀H₉₂FN₁₇O₁₆: 1325.69, found [M+2H]²⁺: 664.1.

### Example 458: (Compound 458) (SEQ ID NO: 611)

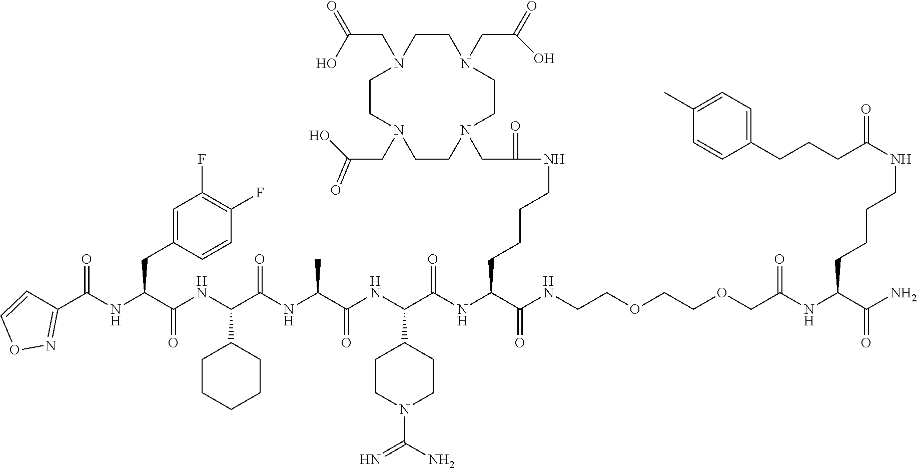 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)C2CCCCC2)C2CCN(C(=N)N)CC2)C(N)=O)cc1]

MS: Calc'd for C₇₇H₁₁₆F₂N₁₈O₁₉: 1634.86, found [M+2H]²⁺: 818.65.

### Example 459: (Compound 459) (SEQ ID NO: 612)

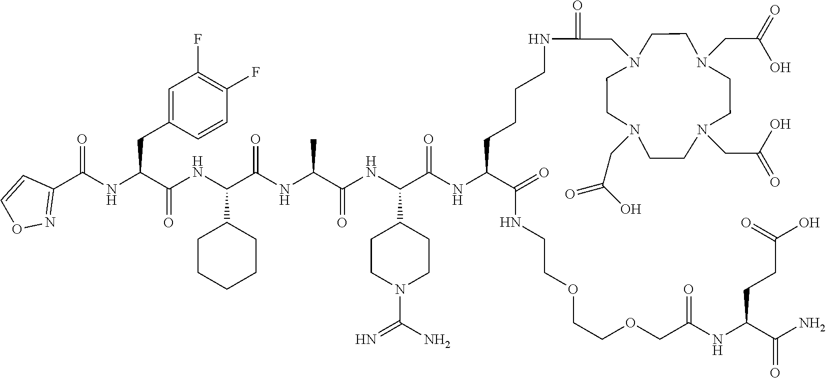 [SMILES: C[C@H](NC(=O)[C@@H](NC(=O)[C@H](Cc1ccc(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCC(=O)O)C(N)=O)C1CCN(C(=N)N)CC1]

MS: Calc'd for C₆₅H₉₉F₂N₁₇O₂₀: 1475.72, found [M+2H]²⁺: 739.25.

### Example 460A: (Compound 460A) (SEQ ID NO: 613)

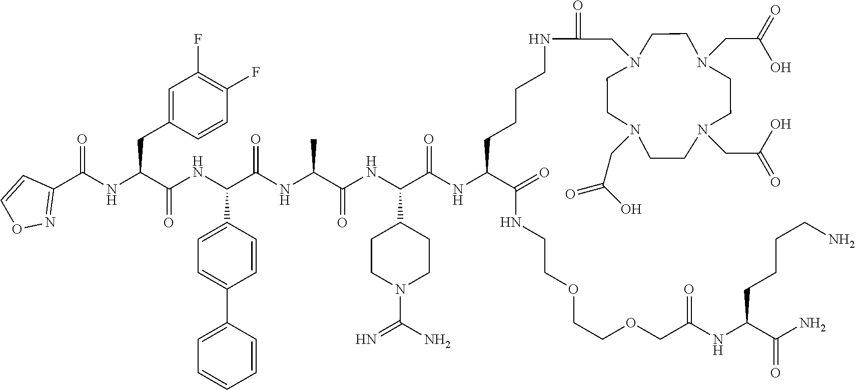 [SMILES: C[C@H](NC(=O)[C@@H](NC(=O)[C@H](Cc1ccc(F)c(F)c1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCN(C(=N)N)CC1]

MS: Calc'd for C₇₂H₁₀₂F₂N₁₈O₁₈: 1544.76, found [M+2H]²⁺: 773.65.

### Example 460B: (Compound 460B) (SEQ ID NO: 614)

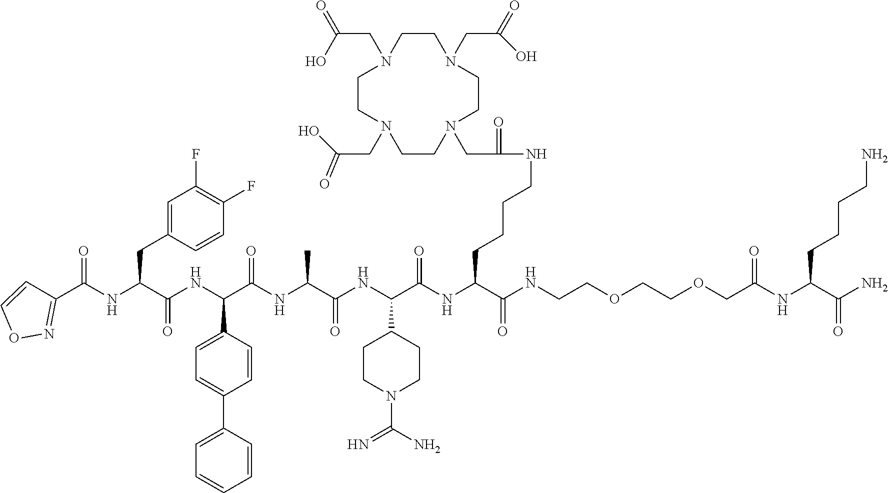 [SMILES: C[C@H](NC(=O)[C@H](NC(=O)[C@H](Cc1ccc(F)c(F)c1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCN(C(=N)N)CC1]

MS: Calc'd for C₇₂H₁₀₂F₂N₁₈O₁₈: 1544.76, found [M+2H]²⁺: 773.65.

### Example 461: (Compound 461) (SEQ ID NO: 615)

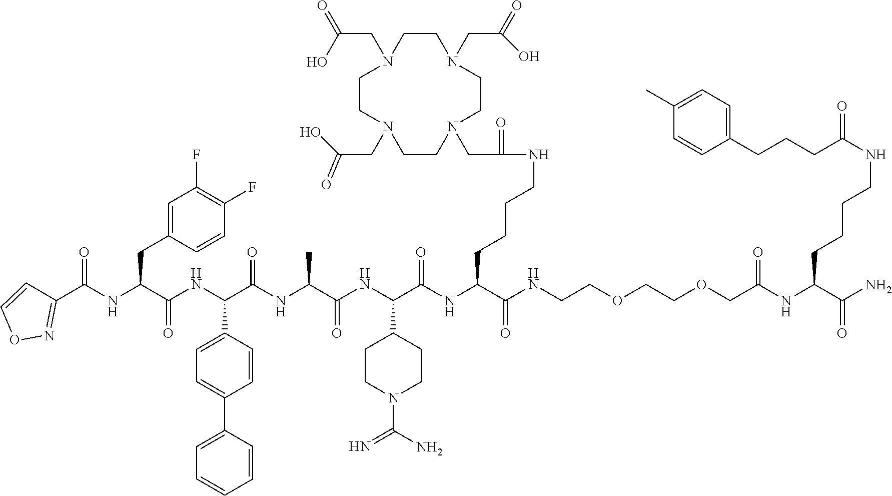 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)c2ccc(-c3ccccc3)cc2)C2CCN(C(=N)N)CC2)C(N)=O)cc1]

MS: Calc'd for C₈₃H₁₁₄F₂N₁₈O₁₉: 1704.85, found [M+2H]²⁺: 853.65.

### Example 462: (Compound 462) (SEQ ID NO: 616)

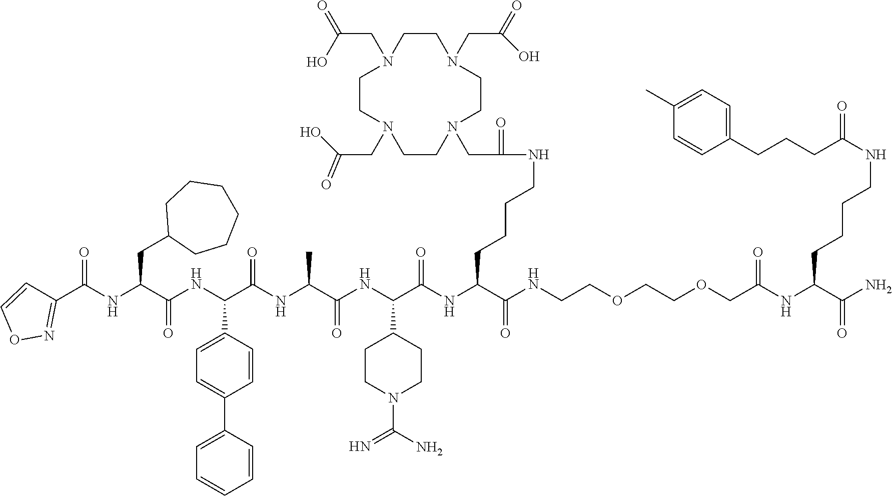 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](CC2CCCCCC2)NC(=O)c2ccon2)c2ccc(-c3ccccc3)cc2)C2CCN(C(=N)N)CC2)C(N)=O)cc1]

### Example 463: (Compound 463) (SEQ ID NO: 617)

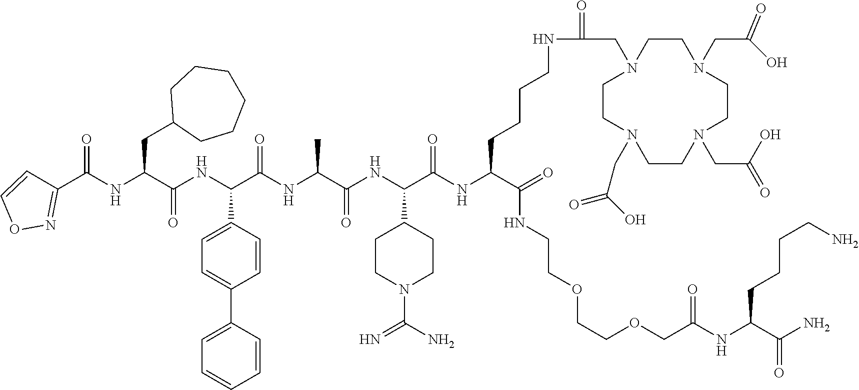 [SMILES: C[C@H](NC(=O)[C@@H](NC(=O)[C@H](CC1CCCCCC1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCN(C(=N)N)CC1]

### Example 464: (Compound 464) (SEQ ID NO: 618)

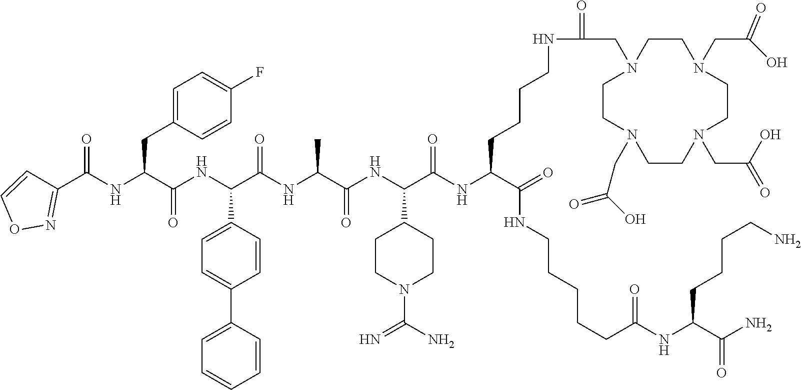 [SMILES: C[C@H](NC(=O)[C@@H](NC(=O)[C@H](Cc1ccc(F)cc1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

### Example 465: (Compound 465) (SEQ ID NO: 619)

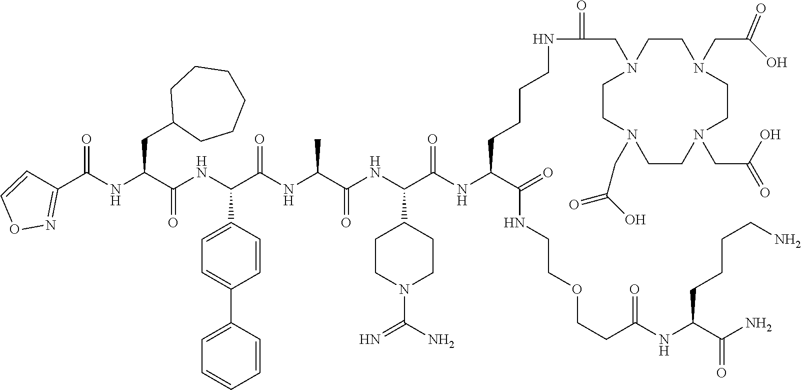 [SMILES: C[C@H](NC(=O)[C@@H](NC(=O)[C@H](CC1CCCCCC1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCOCCC(=O)N[C@@H](CCCCN)C(N)=O)C1CCN(C(=N)N)CC1]

### Example 466: (Compound 466) (SEQ ID NO: 620)

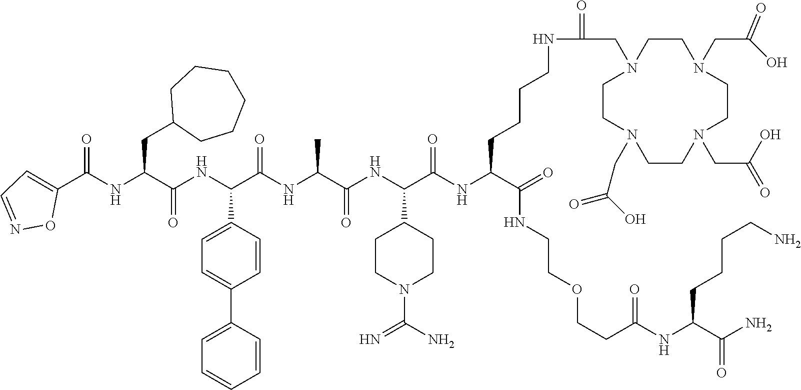 [SMILES: C[C@H](NC(=O)[C@@H](NC(=O)[C@H](CC1CCCCCC1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCOCCC(=O)N[C@@H](CCCCN)C(N)=O)C1CCN(C(=N)N)CC1]

MS: Calc'd for C₇₃H₁₁₁N₁₇O₁₇: 1497.83, found [M+2H]²⁺: 750.25.

### Example 467: (Compound 467) (SEQ ID NO: 621)

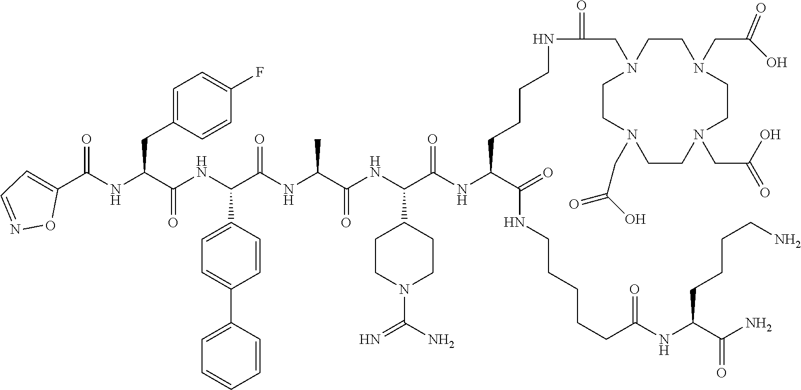 [SMILES: C[C@H](NC(=O)[C@@H](NC(=O)[C@H](Cc1ccc(F)cc1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₇₂H₁₀₂FN₁₇O₁₇: 1495.76, found [M+2H]²⁺: 749.2.

### Example 468: (Compound 468) (SEQ ID NO: 622)

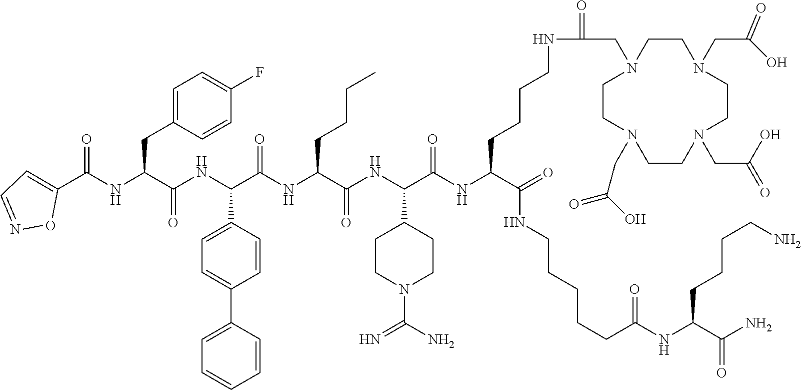 [SMILES: CCCC[C@H](NC(=O)[C@@H](NC(=O)[C@H](Cc1ccc(F)cc1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₇₅H₁₀₈FN₁₇O₁₇: 1537.81, found [M+2H]²⁺: 770.2.

### Example 469: (Compound 469) (SEQ ID NO: 623)

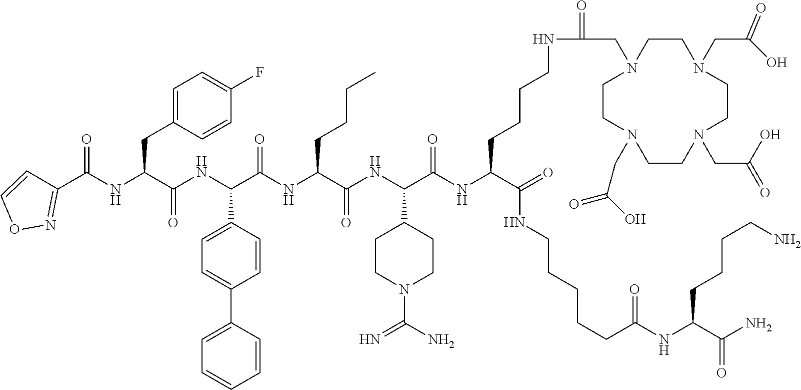 [SMILES: CCCC[C@H](NC(=O)[C@@H](NC(=O)[C@H](Cc1ccc(F)cc1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₇₅H₁₀₅FN₁₇O₁₇: 1537.81, found [M+2H]²⁺: 770.2.

### Example 470: (Compound 470) (SEQ ID NO: 624)

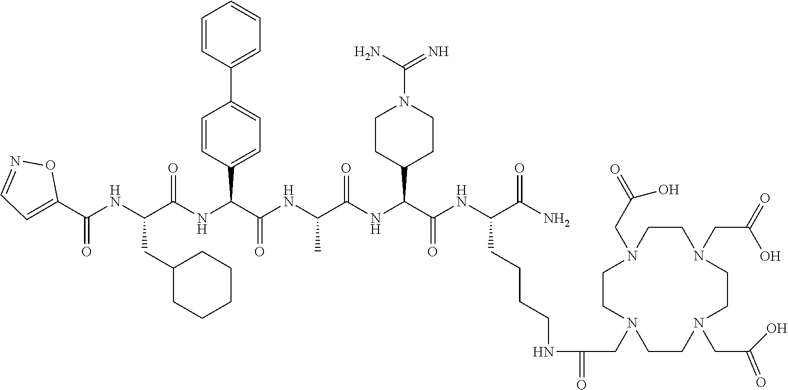 [SMILES: C[C@H](NC(=O)[C@@H](NC(=O)[C@H](CC1CCCCC1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₀H₈₇N₁₅O₁₄: 1241.66, found [M+2H]²⁺: 622.1.

### Example 471: (Compound 471) (SEQ ID NO: 625)

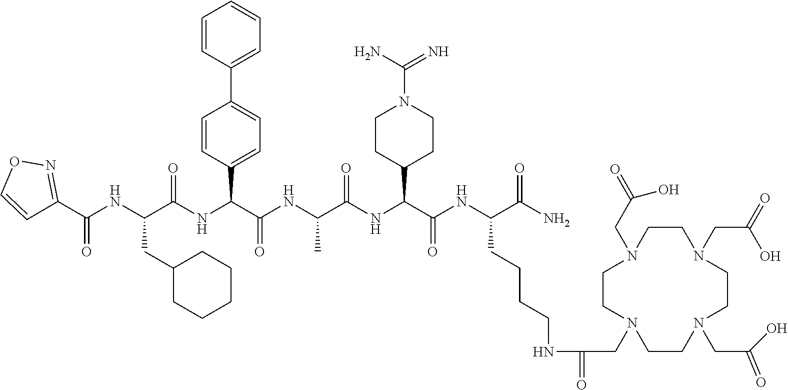 [SMILES: C[C@H](NC(=O)[C@@H](NC(=O)[C@H](CC1CCCCC1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₀H₈₇N₁₅O₁₄: 1241.66, found [M+2H]²⁺: 622.1.

### Example 472: (Compound 472) (SEQ ID NO: 626)

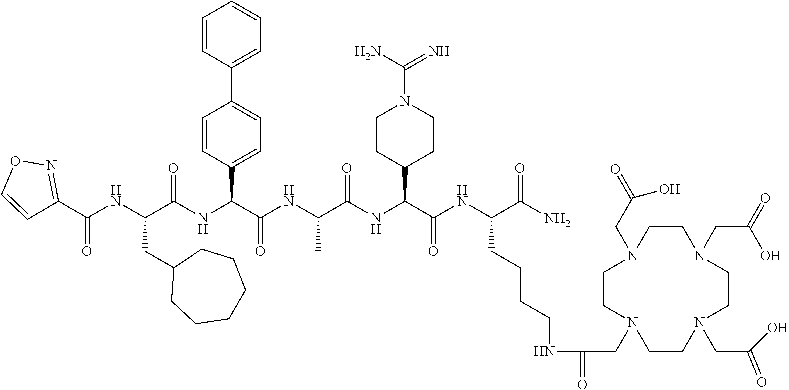 [SMILES: C[C@H](NC(=O)[C@@H](NC(=O)[C@H](CC1CCCCCC1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₁H₈₉N₁₅O₁₄: 1255.67, found [M+2H]²⁺: 629.1.

### Example 473: (Compound 473) (SEQ ID NO: 627)

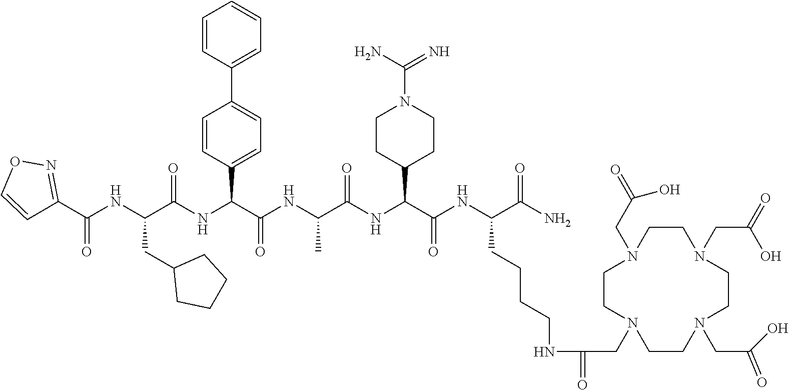 [SMILES: C[C@H](NC(=O)[C@@H](NC(=O)[C@H](CC1CCCC1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₉H₈₅N₁₅O₁₄: 1227.64, found [M+2H]²⁺: 615.1.

### Example 474: (Compound 474) (SEQ ID NO: 628)

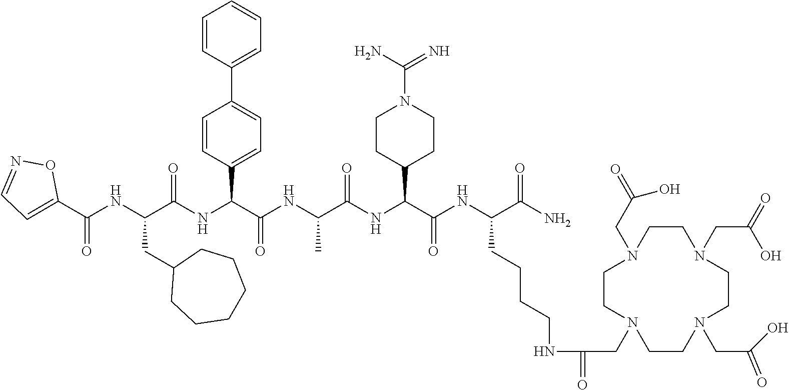 [SMILES: C[C@H](NC(=O)[C@@H](NC(=O)[C@H](CC1CCCCCC1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₆₁H₈₉N₁₅O₁₄: 1255.67, found [M+2H]²⁺: 629.1.

### Example 475: (Compound 475) (SEQ ID NO: 629)

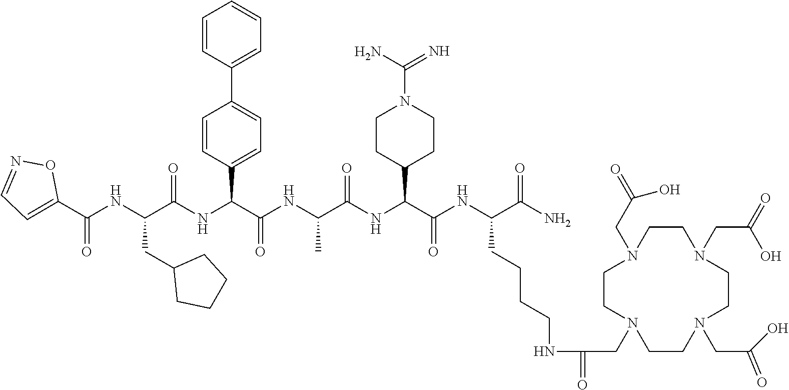 [SMILES: C[C@H](NC(=O)[C@@H](NC(=O)[C@H](CC1CCCC1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

MS: Calc'd for C₅₉H₈₅N₁₅O₁₄: 1227.64, found [M+2H]²⁺: 615.05.

### Example 476: (Compound 476) (SEQ ID NO: 630)

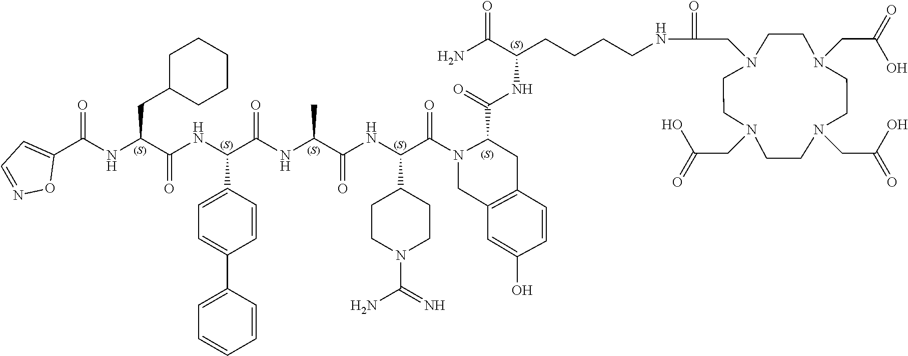

### Example 477: (Compound 477) (SEQ ID NO: 631)

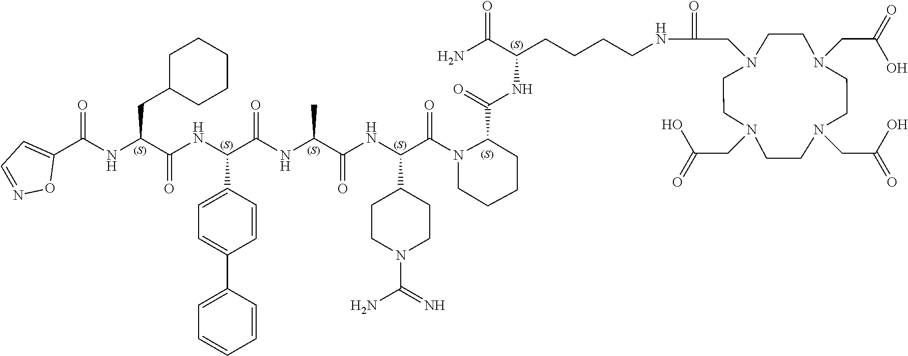

MS: Calc'd for C₆₆H₉₆N₁₆O₁₅: 1352.72, found [M+2H]²⁺: 677.55.

### Example 478: (Compound 478) (SEQ ID NO: 632)

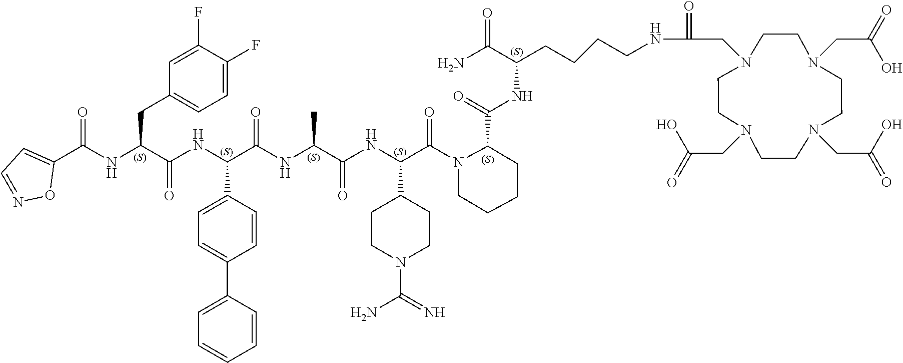

MS: Calc'd for C₆₆H₈₈F₂N₁₆O₁₅: 1382.66, found [M+2H]²⁺: 692.65.

### Example 479: (Compound 479) (SEQ ID NO: 633)

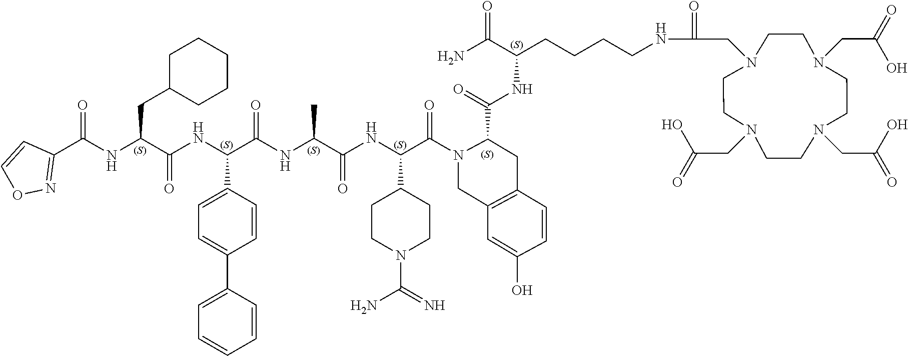

### Example 480: (Compound 480) (SEQ ID NO: 634)

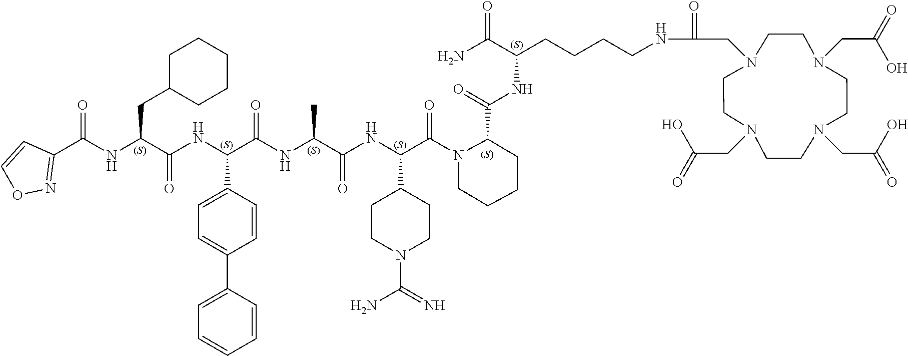

MS: Calc'd for C₆₆H₉₆N₁₆O₁₅: 1352.72, found [M+2H]²⁺: 677.6.

### Example 481: (Compound 481) (SEQ ID NO: 635)

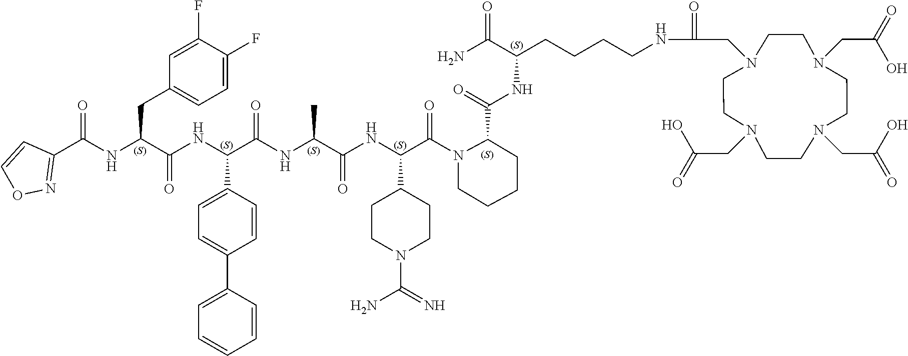

MS: Calc'd for C₆₆H₈₃F₂N₁₆O₁₅: 1382.66, found [M+2H]²⁺: 692.7.

### Example 482: (Compound 482) (SEQ ID NO: 636)

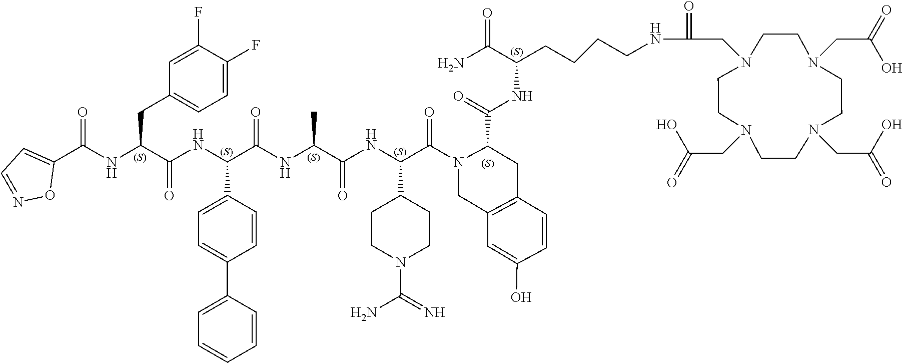

### Example 483: (Compound 483) (SEQ ID NO: 637)

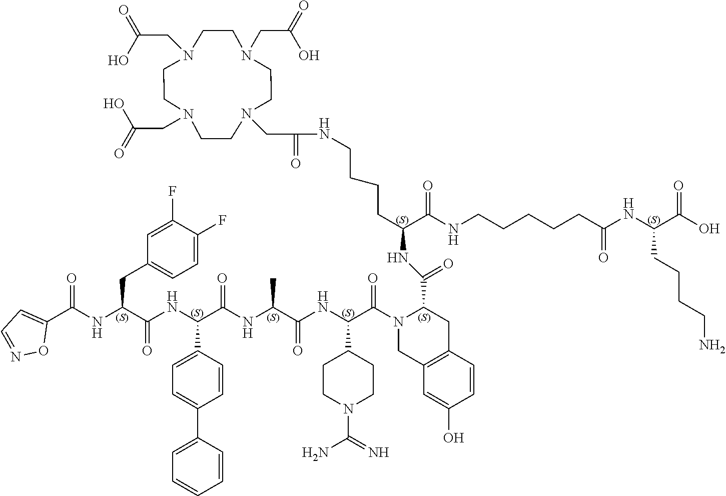

### Example 484: (Compound 484) (SEQ ID NO: 638)

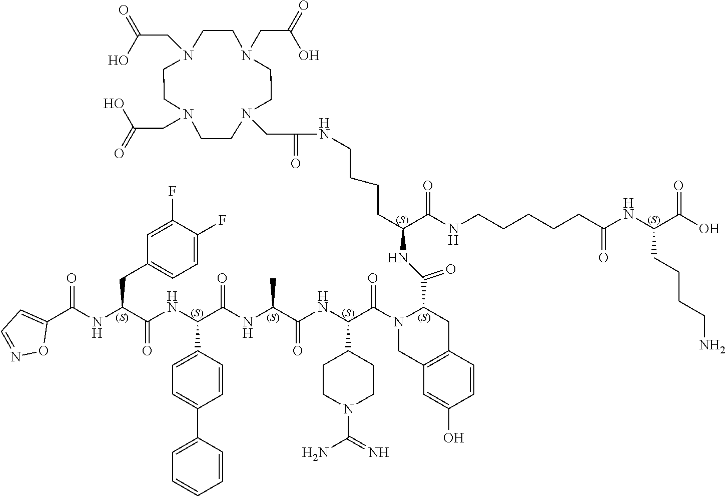

MS: Calc'd for C₈₂H₁₁₀F₂N₁₈O₁₉: 1688.82, found [M+2H]²⁺: 845.75.

### Example 485: (Compound 485) (SEQ ID NO: 639)

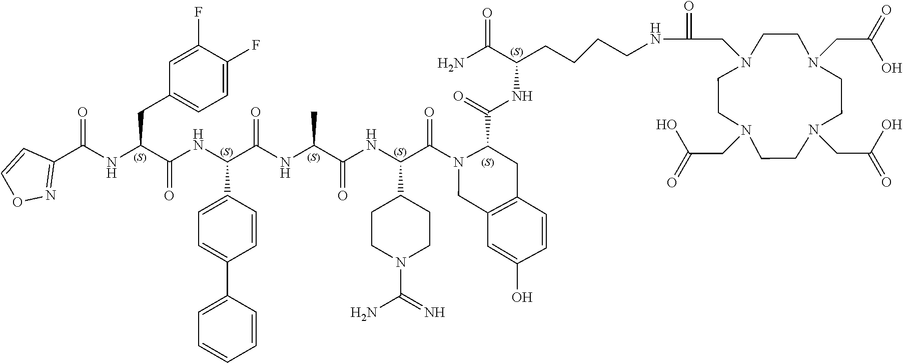

MS: Calc'd for C₇₀H₈₈F₂N₁₆O₁₆: 1446.65, found [M+H]⁺: 1447.1.

### Example 486: (Compound 486) (SEQ ID NO: 640)

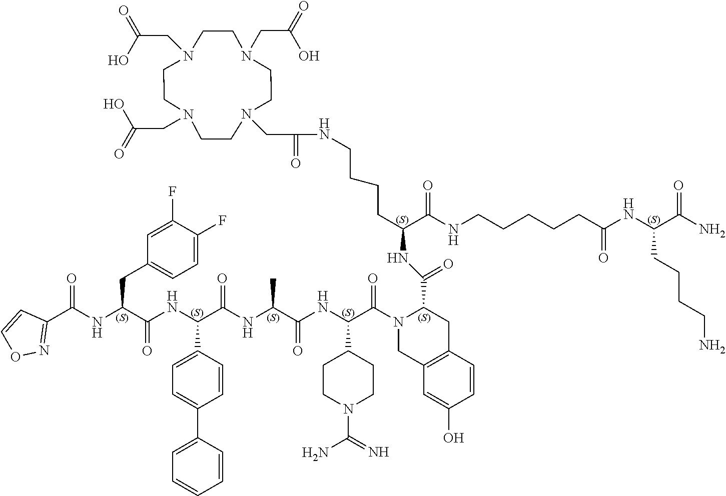

MS: Calc'd for C₈₂H₁₁₀F₂N₁₈O₁₉: 1688.82, found [M+2H]²⁺: 845.7.

### Example 487: (Compound 487) (SEQ ID NO: 641)

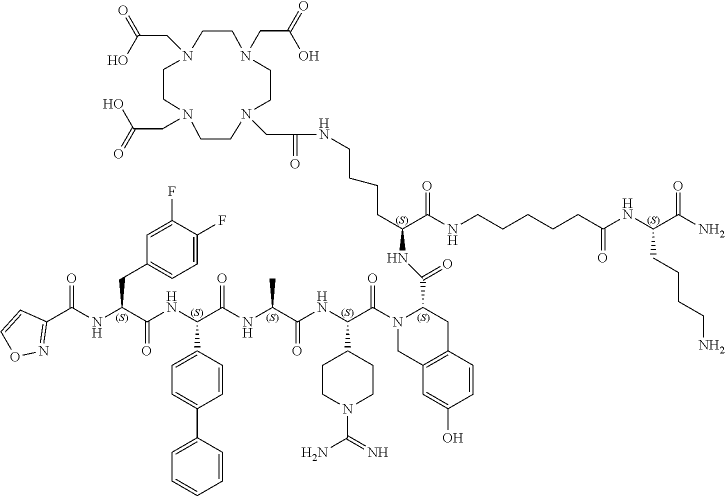

### Example 488: (Compound 488) (SEQ ID NO: 642)

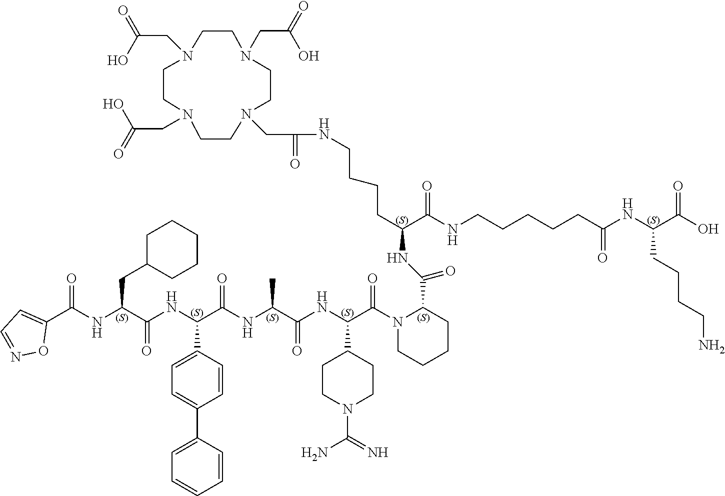

MS: Calc'd for C₇₈H₁₁₈N₁₈O₁₅: 1594.89, found [M+2H]²⁺: 798.75.

### Example 489: (Compound 489) (SEQ ID NO: 643)

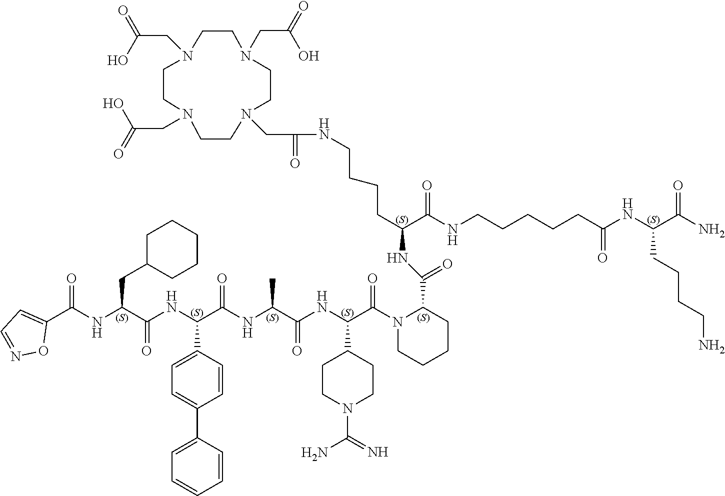

MS: Calc'd for C₇₈H₁₁₉N₁₉O₁₇:1593.90, found [M+2H]²⁺: 798.3.

### Example 490: (Compound 490) (SEQ ID NO: 644)

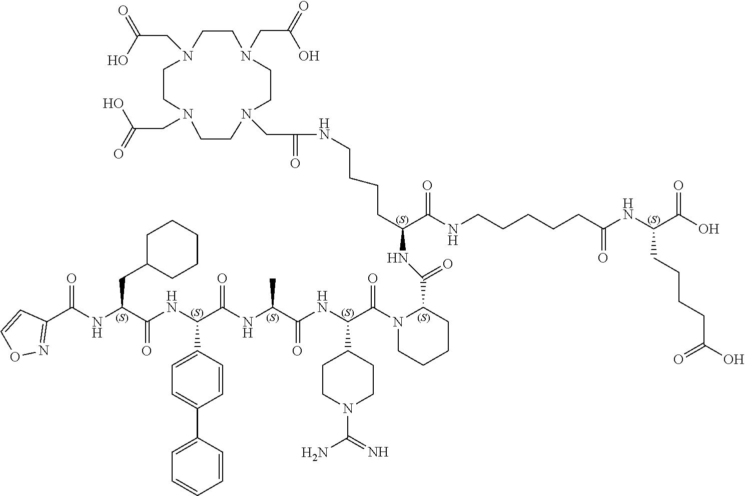

MS: Calc'd for C₇₇H₁₁₃N₁₇O₂₀: 1595.83, found [M+H]⁺: 1595.3.

### Example 491: (Compound 491) (SEQ ID NO: 645)

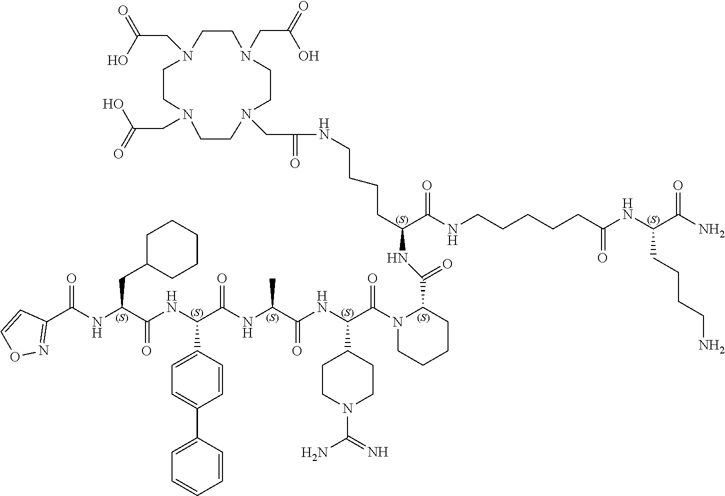

MS: Calc'd for C₇₈H₁₁₉N₁₉O₁₇: 1593.90, found [M+2H]²⁺: 798.2.

### Example 492: (Compound 492) (SEQ ID NO: 646)

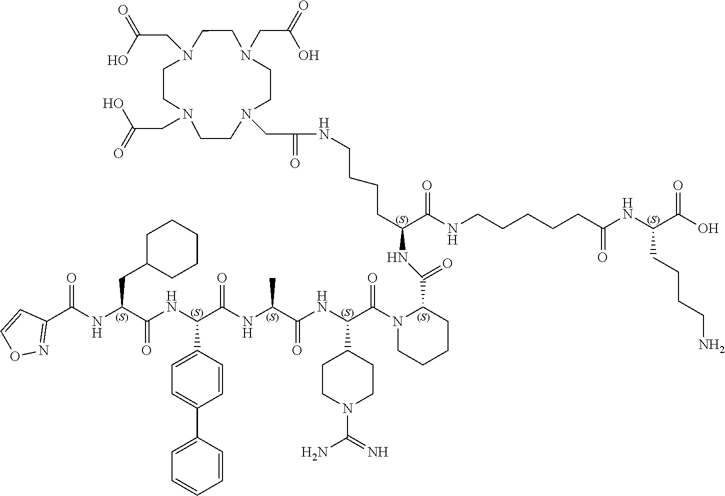

### Example 493: (Compound 493) (SEQ ID NO: 647)

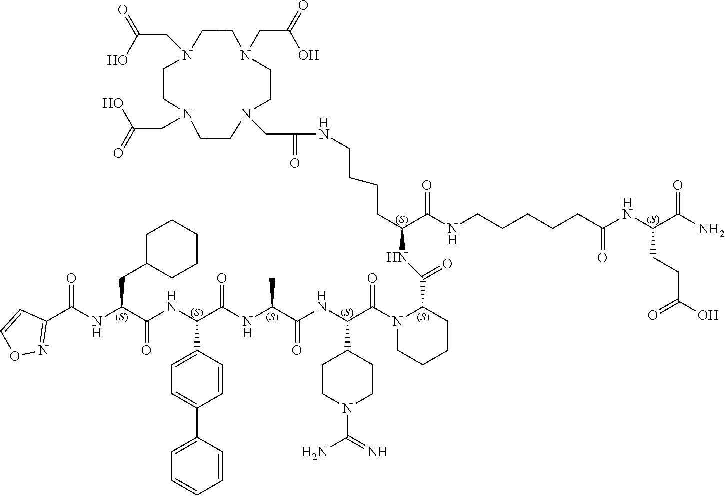

MS: Calc'd for C₇₇H₁₁₄N₁₈O₁₉: 1594.85, found [M+2H]²⁺: 798.75.

### Example 494: (Compound 494) (SEQ ID NO: 648)

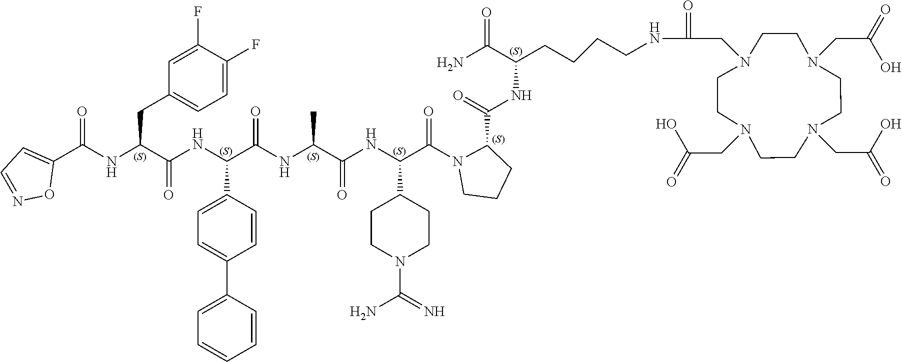

MS: Calc'd for C₆₅H₈₆F₂N₁₆O₁₅: 1368.64, found [M+H]⁺: 1369.2.

### Example 495: (Compound 495) (SEQ ID NO: 649)

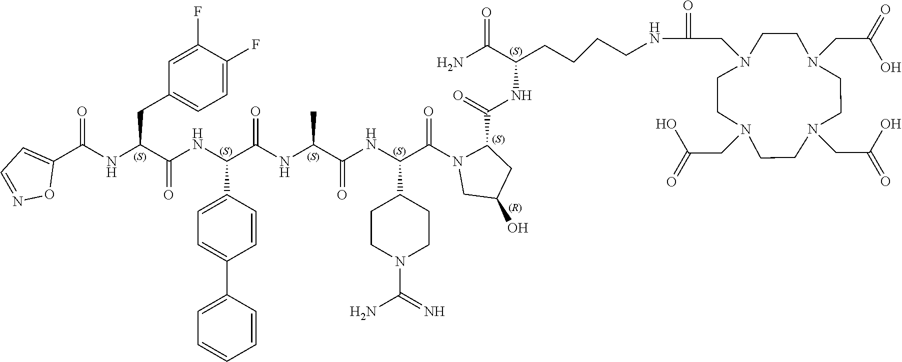

MS: Calc'd for C₆₅H₈₆F₂N₁₆O₁₆: 1384.64, found [M+H]⁺: 1385.15.

### Example 496: (Compound 496) (SEQ ID NO: 650)

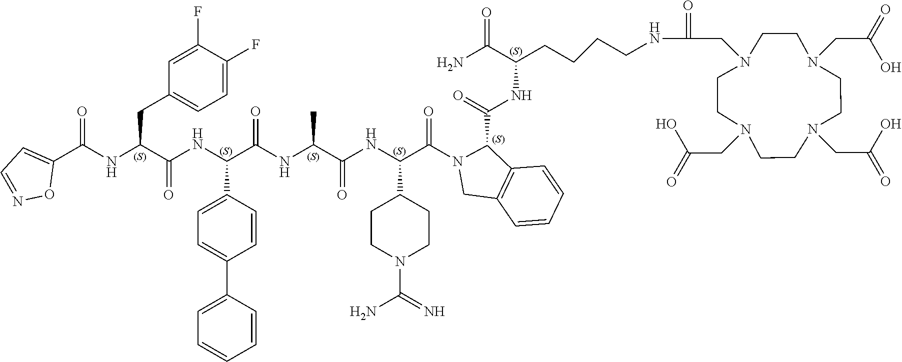

### Example 497: (Compound 497) (SEQ ID NO: 651)

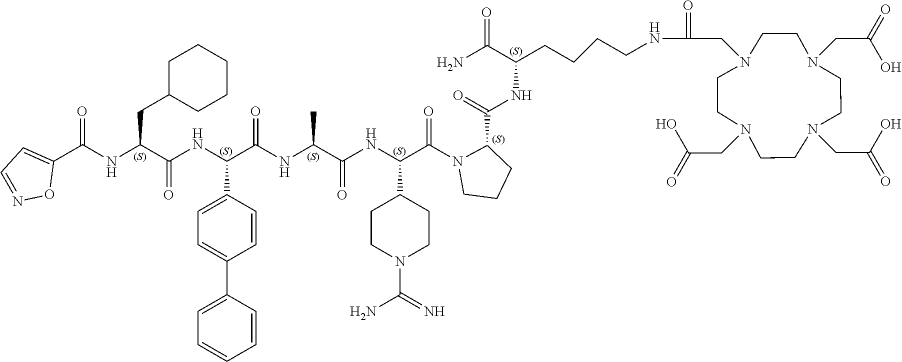

MS: Calc'd for C₆₅H₉₄N₁₆O₁₅: 1338.71, found [M+2H]²⁺: 670.6.

### Example 498: (Compound 498) (SEQ ID NO: 652)

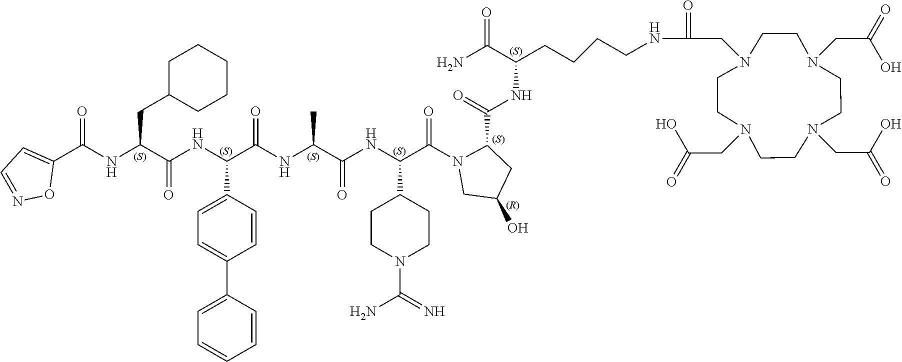

MS: Calc'd for C₆₅H₉₄N₁₆O₁₆: 1354.70, found [M+2H]²⁺: 678.6.

### Example 499: (Compound 499) (SEQ ID NO: 653)

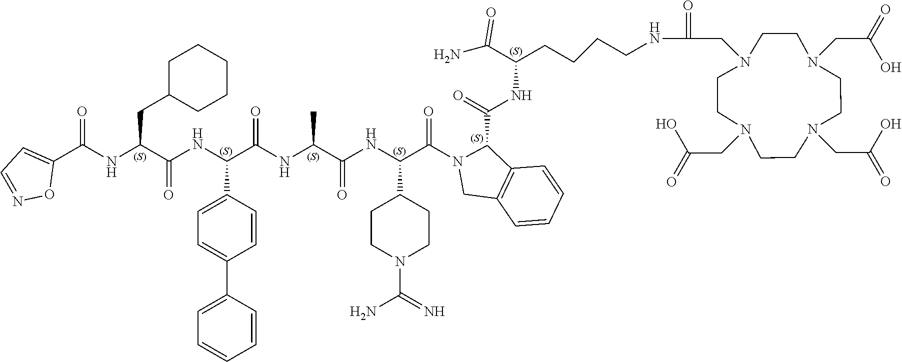

### Example 500: (Compound 500) (SEQ ID NO: 654)

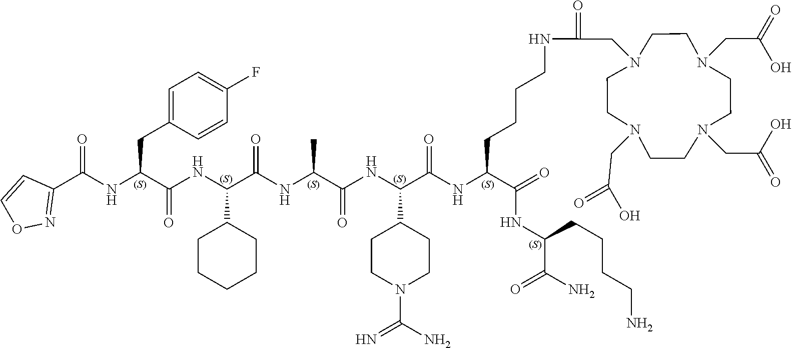

### Example 501: (Compound 501) (SEQ ID NO: 655)

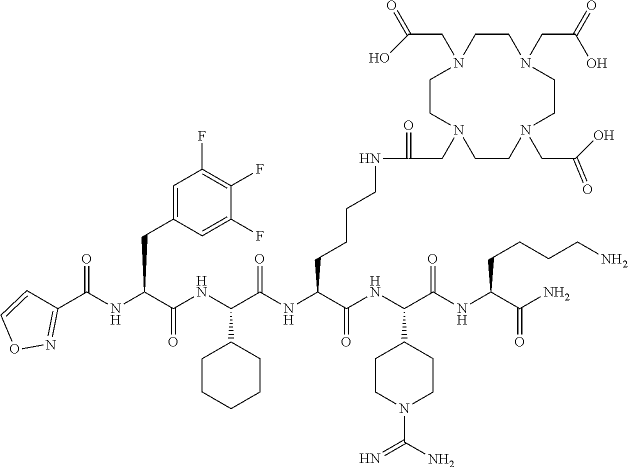 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@H](Cc2cc(F)c(F)c(F)c2)NC(=O)c2ccon2)C2CCCCC2)C(=O)N[C@@H](CCCCN)C(N)=O)CC1]

### Example 502: (Compound 502) (SEQ ID NO: 656)

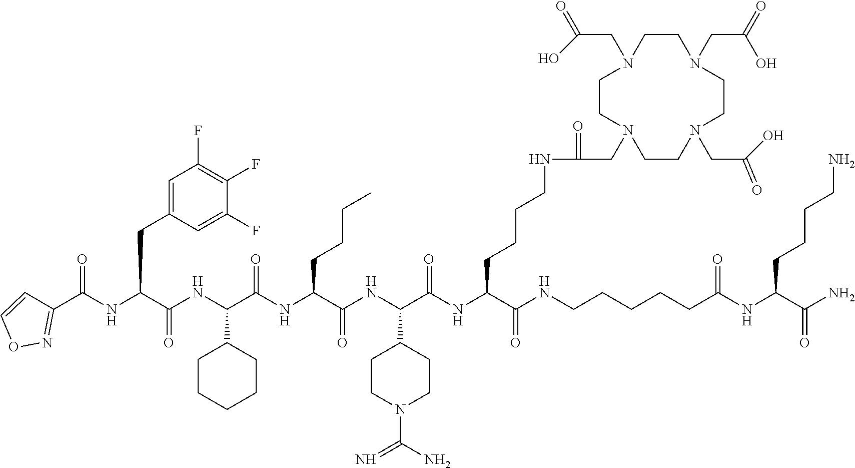 [SMILES: CCCC[C@H](NC(=O)[C@@H](NC(=O)[C@H](Cc1cc(F)c(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

### Example 503: (Compound 503) (SEQ ID NO: 657)

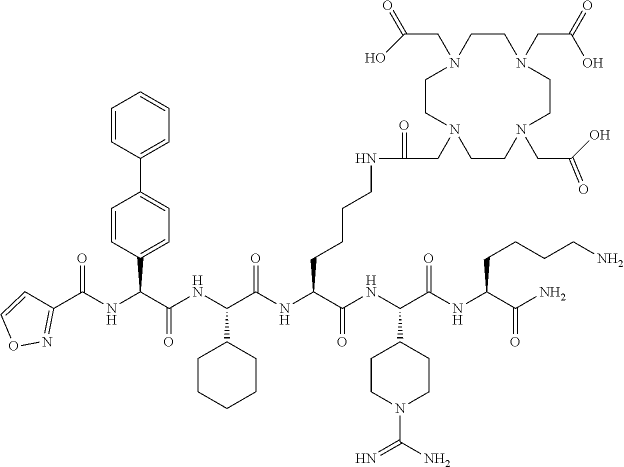 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@@H](NC(=O)c2ccon2)c2ccc(-c3ccccc3)cc2)C2CCCCC2)C(=O)N[C@@H](CCCCN)C(N)=O)CC1]

### Example 504A: (Compound 504A) (SEQ ID NO: 658)

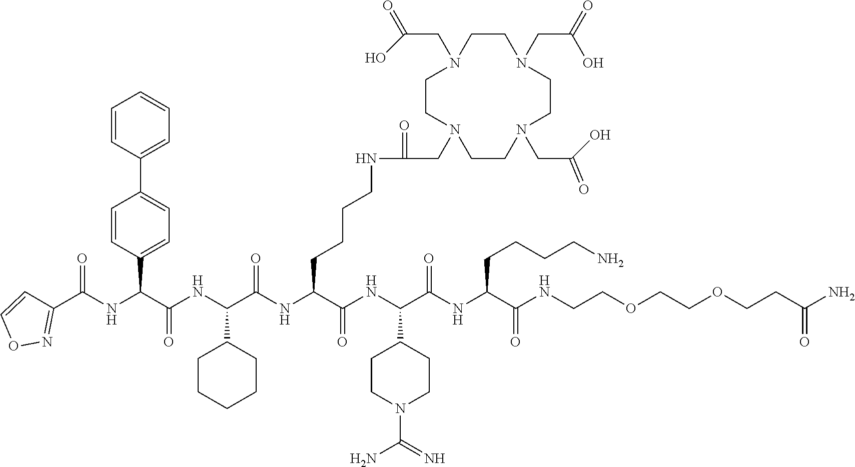 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@@H](NC(=O)c2ccon2)c2ccc(-c3ccccc3)cc2)C2CCCCC2)C(=O)N[C@@H](CCCCN)C(=O)NCCOCCOCCC(N)=O)CC1]

MS: Calc'd for C₆₉H₁₀₅N₁₇O₁₇: 1443.79, found [M+2H]²⁺: 723.2.

### Example 504B: (Compound 504B) (SEQ ID NO: 659)

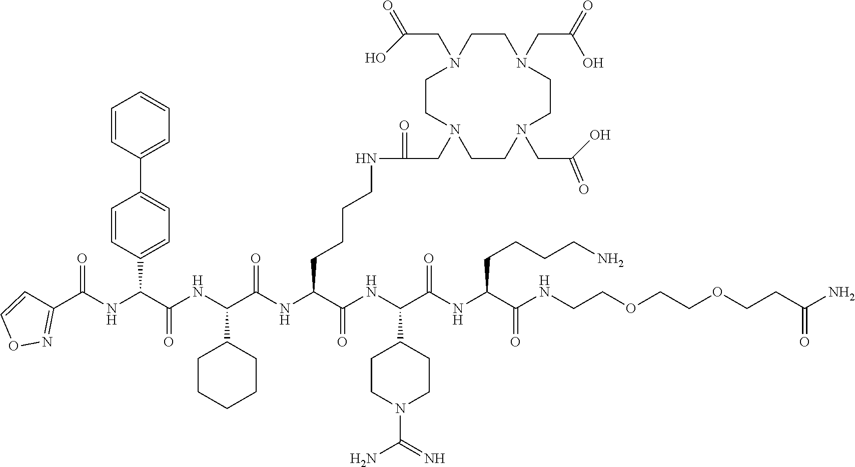 [SMILES: N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@H](NC(=O)c2ccon2)c2ccc(-c3ccccc3)cc2)C2CCCCC2)C(=O)N[C@@H](CCCCN)C(=O)NCCOCCOCCC(N)=O)CC1]

MS: Calc'd for C₆₉H₁₀₅N₁₇O₁₇: 1443.79, found [M+2H]²⁺: 723.2.

### Example 505: (Compound 505) (SEQ ID NO: 660)

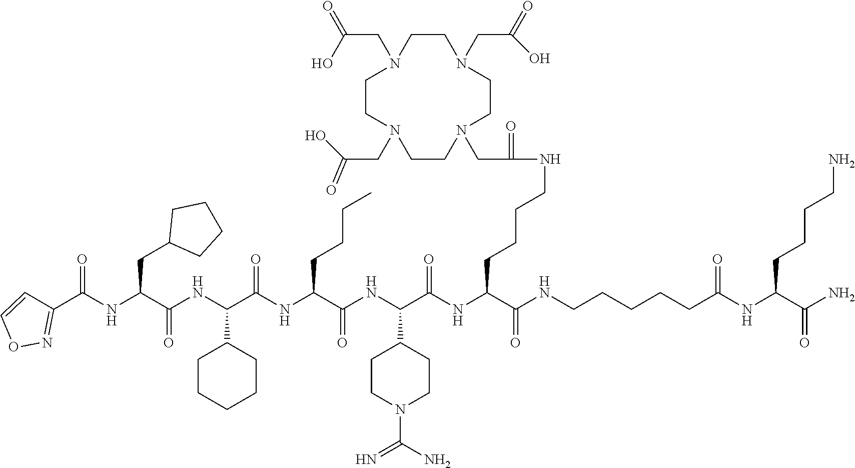 [SMILES: CCCC[C@H](NC(=O)[C@@H](NC(=O)[C@H](CC1CCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1]

MS: Calc'd for C₆₈H₁₁₆N₁₈O₁₆: 1440.88, found [M+2H]²⁺: 722.25.

### Example 506: (Compound 506)

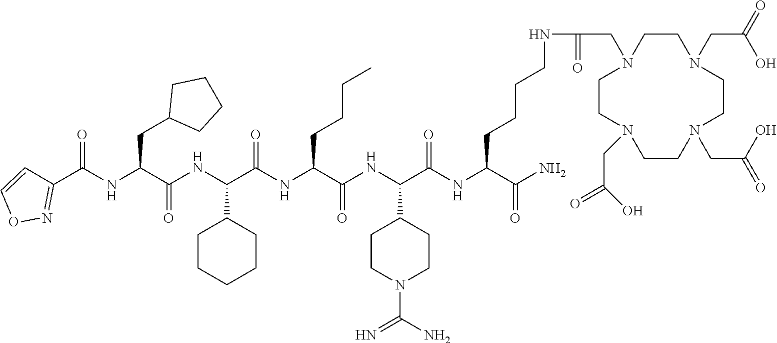 [SMILES: CCCC[C@H](NC(=O)[C@@H](NC(=O)[C@H](CC1CCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

### Example 507: (Compound 507)

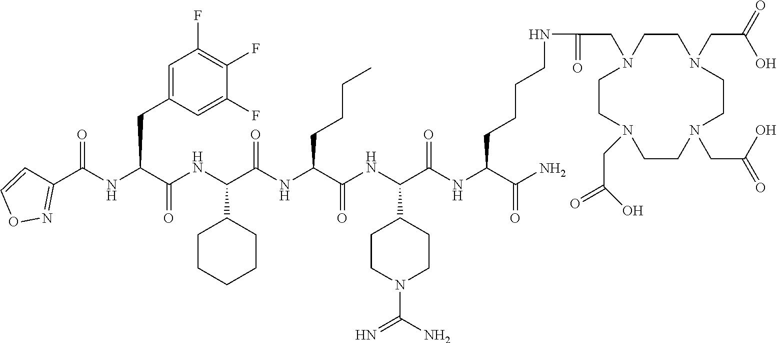 [SMILES: CCCC[C@H](NC(=O)[C@@H](NC(=O)[C@H](Cc1cc(F)c(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

### Example 508: (Compound 508) (SEQ ID NO: 663)

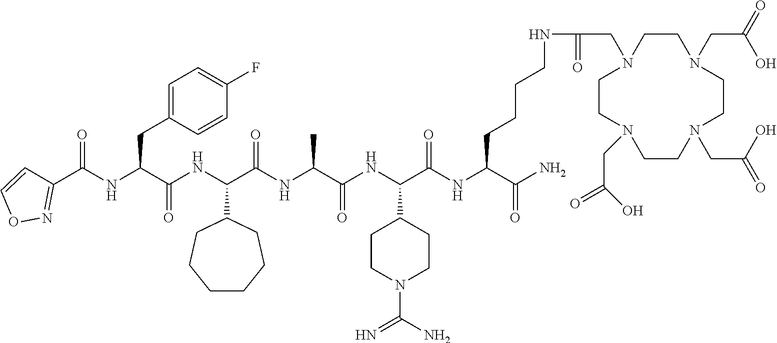 [SMILES: C[C@H](NC(=O)[C@@H](NC(=O)[C@H](Cc1ccc(F)cc1)NC(=O)c1ccon1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

### Example 509: (Compound 509)

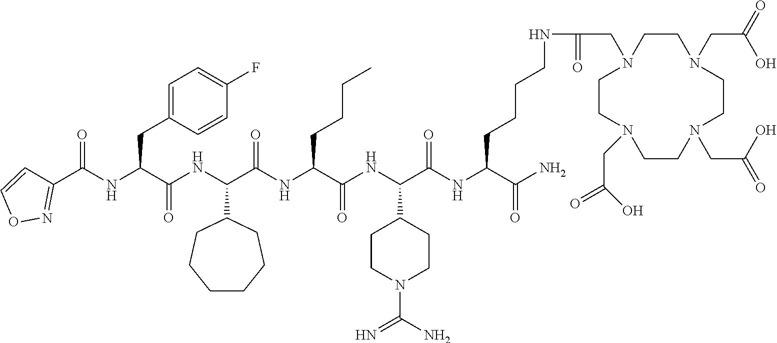 [SMILES: CCCC[C@H](NC(=O)[C@@H](NC(=O)[C@H](Cc1ccc(F)cc1)NC(=O)c1ccon1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

### Example 510: (Compound 510) (SEQ ID NO: 665)

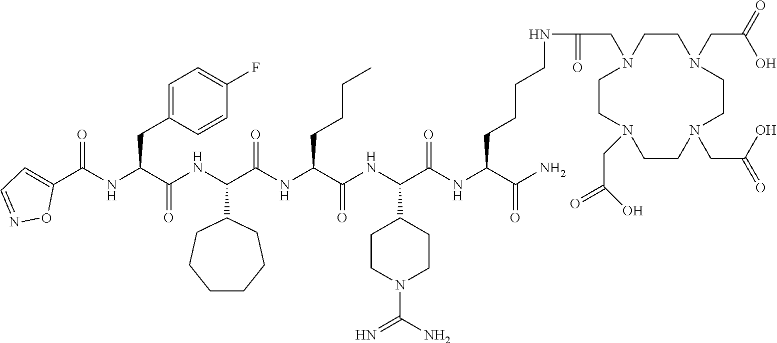 [SMILES: CCCC[C@H](NC(=O)[C@@H](NC(=O)[C@H](Cc1ccc(F)cc1)NC(=O)c1ccno1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

### Example 511: (Compound 511) (SEQ ID NO: 666)

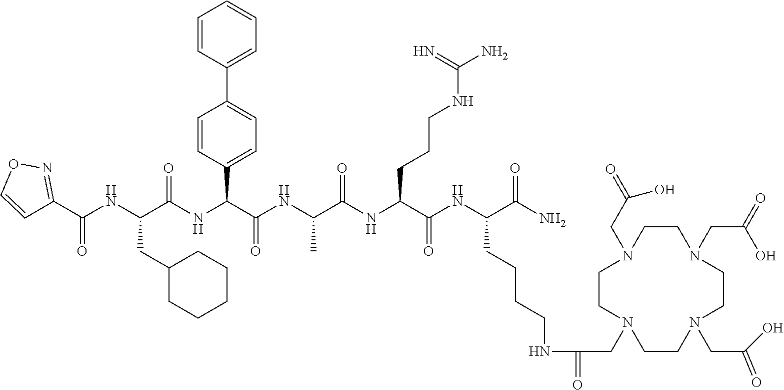 [SMILES: C[C@H](NC(=O)[C@@H](NC(=O)[C@H](CC1CCCCC1)NC(=O)c1ccon1)c1ccc(-c2ccccc2)cc1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O]

### Example 512: (Compound 512) (SEQ ID NO: 667)

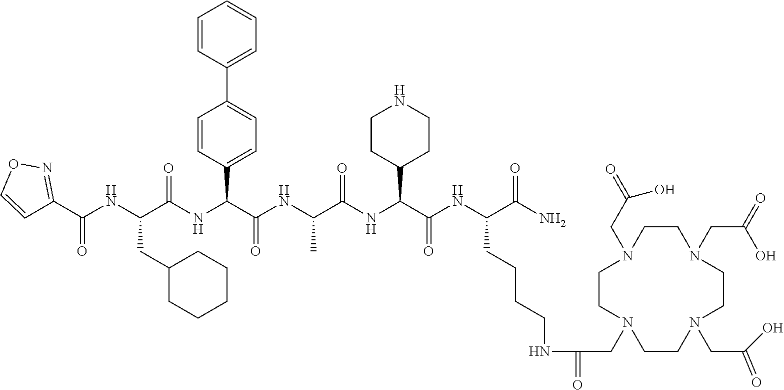 [SMILES: C[C@H](NC(=O)[C@@H](NC(=O)[C@H](CC1CCCCC1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

### Example 513: (Compound 513) (SEQ ID NO: 668)

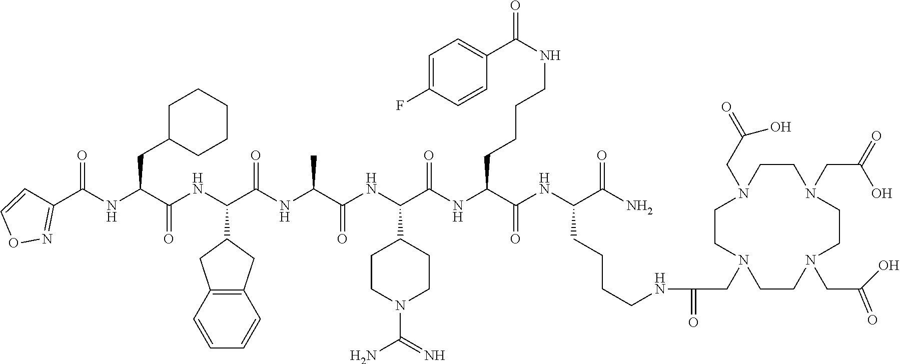 [SMILES: C[C@H](NC(=O)[C@@H](NC(=O)[C@H](CC1CCCCC1)NC(=O)c1ccon1)C1Cc2ccccc2C1)C(=O)N[C@H](C(=O)N[C@@H](CCCCNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1]

### Example 514: (Compound 514) (SEQ ID NO: 669)

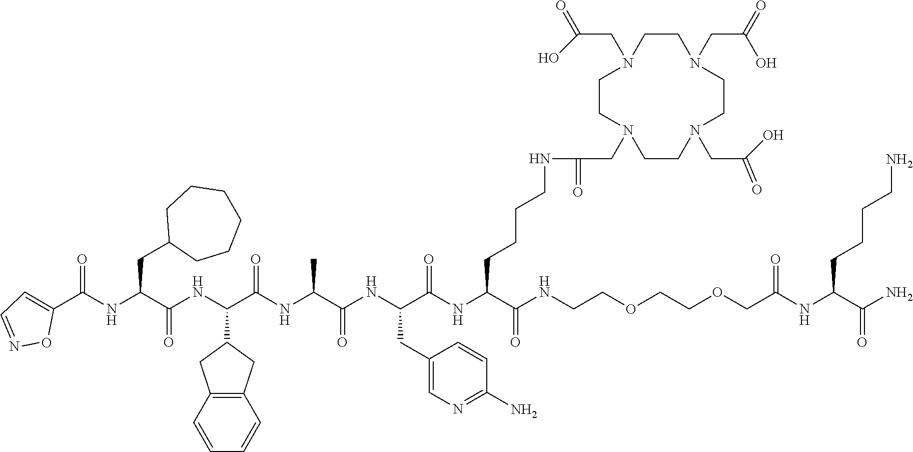 [SMILES: C[C@H](NC(=O)[C@@H](NC(=O)[C@H](CC1CCCCCC1)NC(=O)c1ccno1)C1Cc2ccccc2C1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O]

### Example 515: (Compound 515) (SEQ ID NO: 670)

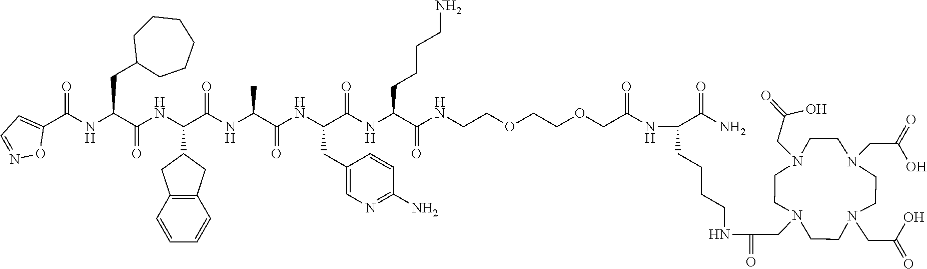 [SMILES: C[C@H](NC(=O)[C@@H](NC(=O)[C@H](CC1CCCCCC1)NC(=O)c1ccno1)C1Cc2ccccc2C1)C(=O)N[C@@H](Cc1ccc(N)nc1)C(=O)N[C@@H](CCCCN)C(=O)NCCOCCOCC(=O)N[C@@H](CCCCNC(=O)CN1CCN(CC(=O)O)CCN(CC(=O)O)CCN(CC(=O)O)CC1)C(N)=O]

### Example 516: (Compound 516) (SEQ ID NO: 671)

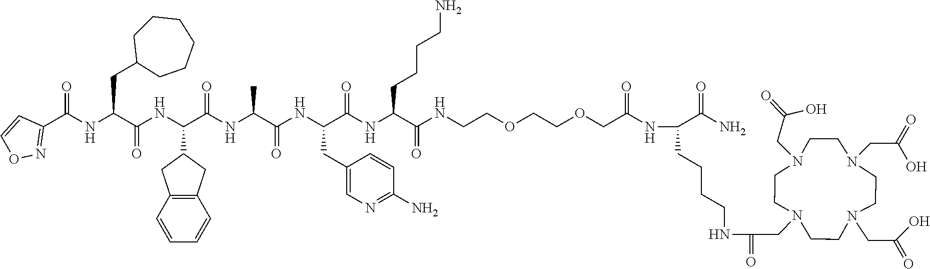 [SMILES: C[C@H](NC(=O)[C@@H](NC(=O)[C@H](CC1CCCCCC1)NC(=O)c1ccon1)C1Cc2ccccc2C1)C(=O)N[C@@H](Cc1ccc(N)nc1)C(=O)N[C@@H](CCCCN)C(=O)NCCOCCOCC(=O)N[C@@H](CCCCNC(=O)CN1CCN(CC(=O)O)CCN(CC(=O)O)CCN(CC(=O)O)CC1)C(N)=O]

### Example 517: (Compound 517) (SEQ ID NO: 672)

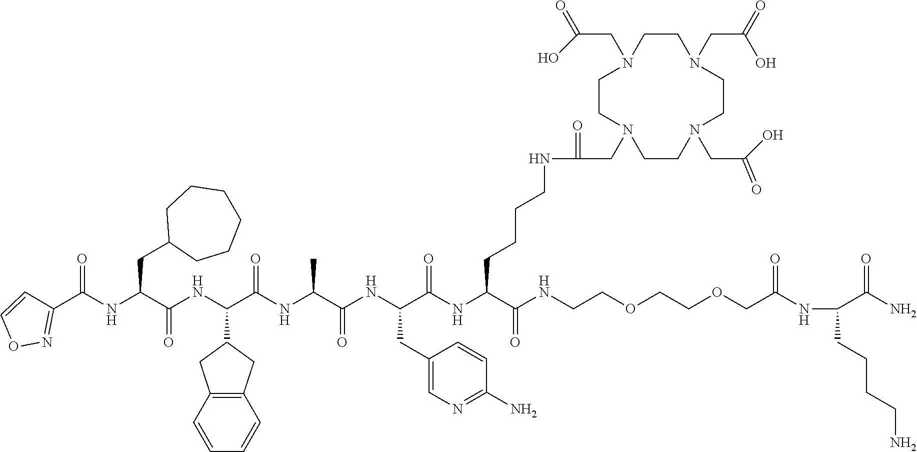 [SMILES: C[C@H](NC(=O)[C@@H](NC(=O)[C@H](CC1CCCCCC1)NC(=O)c1ccon1)C1Cc2ccccc2C1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O]

### Example 518: (Compound 518) (SEQ ID NO: 673)

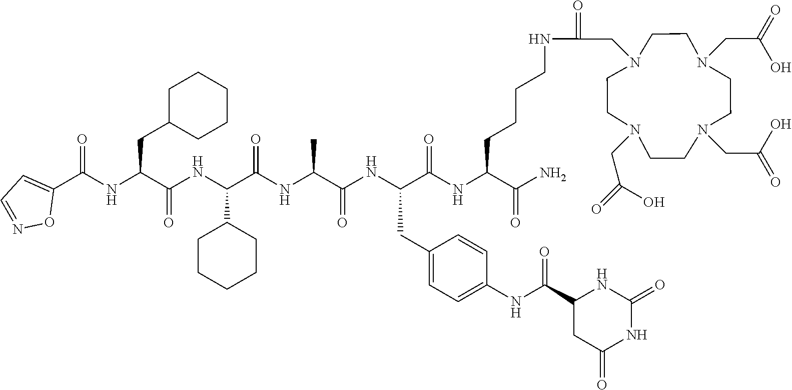

MS: Calc'd for C₆₀H₈₉N₁₅O₁₇: 1291.66, found [M+2H]²⁺: 647.1.

### Example 519: (Compound 519) (SEQ ID NO: 674)

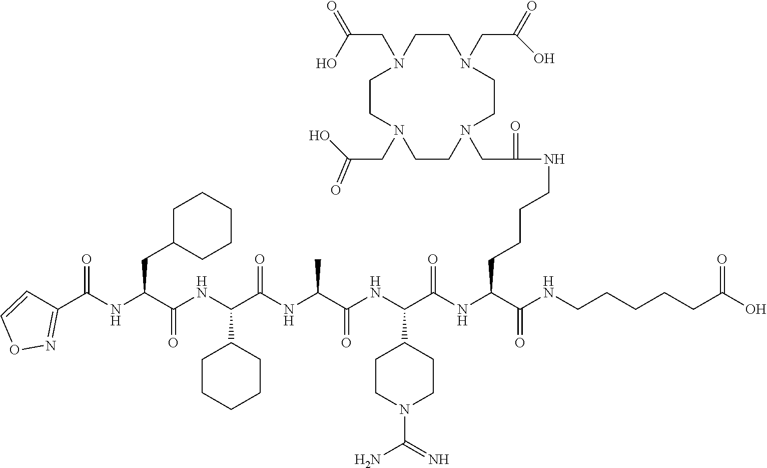 [SMILES: C[C@H](NC(=O)[C@@H](NC(=O)[C@H](CC1CCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)O)C1CCN(C(=N)N)CC1]

MS: Calc'd for C₆₀H₉₉N₁₅O₁₆: 1285.74, found [M+2H]²⁺: 644.15.

### Example 520: (Compound 520) (SEQ ID NO: 675)

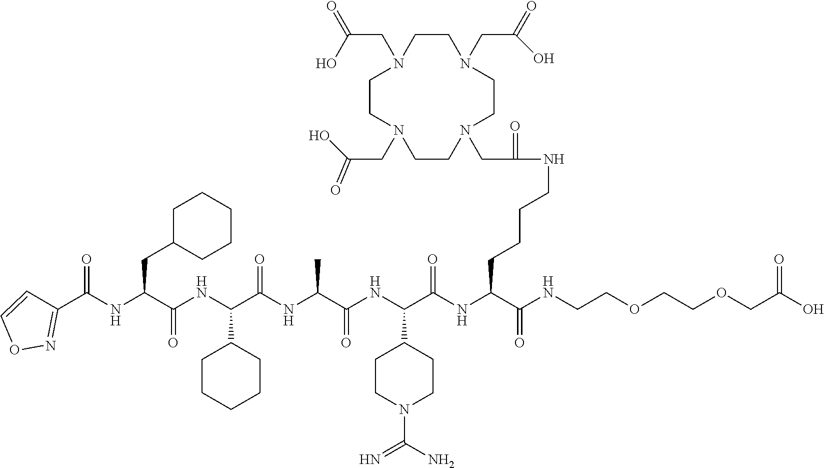 [SMILES: C[C@H](NC(=O)[C@@H](NC(=O)[C@H](CC1CCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)O)C1CCN(C(=N)N)CC1]

MS: Calc'd for C₆₀H₉₉N₁₅O₁₈: 1317.73, found [M+2H]²⁺: 660.15.

### Example 521: (Compound 521) (SEQ ID NO: 676)

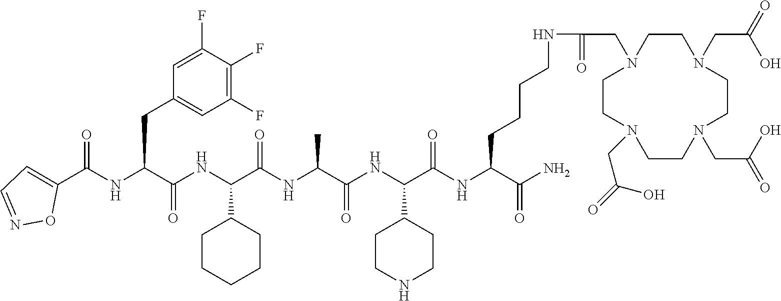 [SMILES: C[C@H](NC(=O)[C@@H](NC(=O)[C@H](Cc1cc(F)c(F)c(F)c1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₅₃H₇₈F₃N₁₃O₁₄: 1177.57, found [M+2H]²⁺: 590.05.

### Example 522: (Compound 522) (SEQ ID NO: 677)

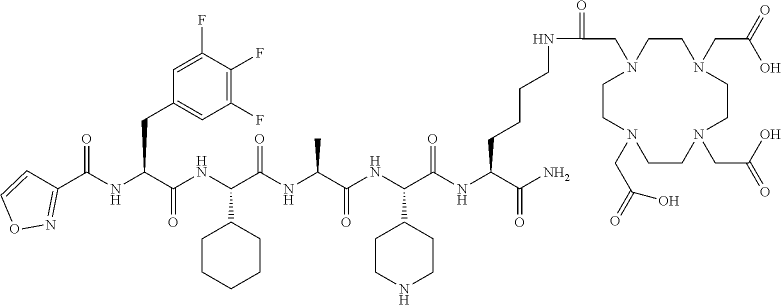 [SMILES: C[C@H](NC(=O)[C@@H](NC(=O)[C@H](Cc1cc(F)c(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₅₃H₇₈F₃N₁₃O₁₄: 1177.57, found [M+2H]²⁺: 590.1.

### Example 523: (Compound 523) (SEQ ID NO: 678)

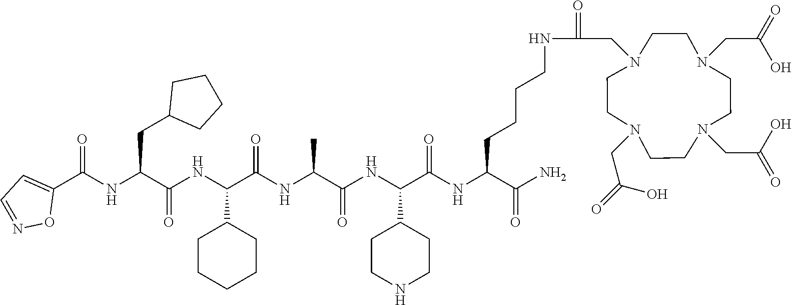 [SMILES: C[C@H](NC(=O)[C@@H](NC(=O)[C@H](CC1CCCC1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₅₂H₈₅N₁₃O₁₄: 1115.64, found [M+2H]²⁺: 559.05.

### Example 524: (Compound 524) (SEQ ID NO: 679)

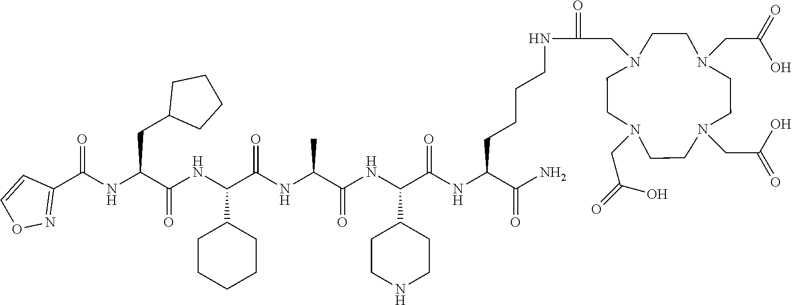 [SMILES: C[C@H](NC(=O)[C@@H](NC(=O)[C@H](CC1CCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(N)=O)C1CCNCC1]

MS: Calc'd for C₅₂H₈₅N₁₃O₁₄: 1115.63, found [M+2H]²⁺: 559.1.

### Example 525: (Compound 525) (SEQ ID NO: 680)

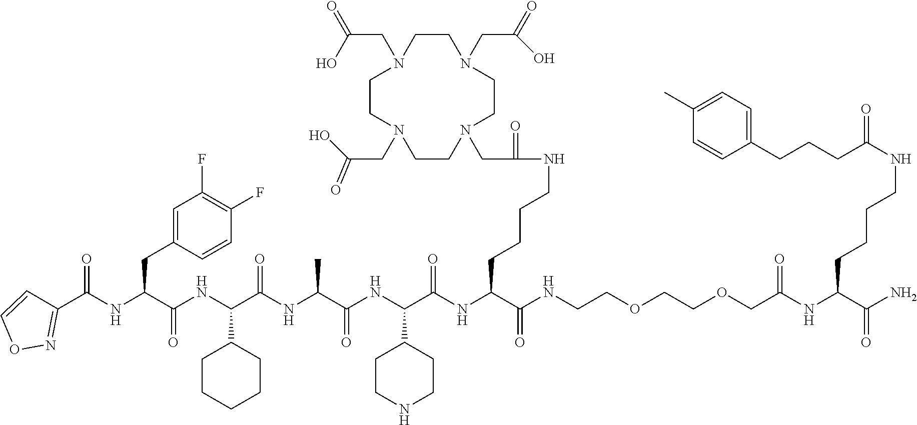 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)C2CCCCC2)C2CCNCC2)C(N)=O)cc1]

MS: Calc'd for C₇₆H₁₁₄F₂N₁₆O₁₉: 1592.84, found [M+2H]²⁺: 797.75.

### Example 526: (Compound 526) (SEQ ID NO: 681)

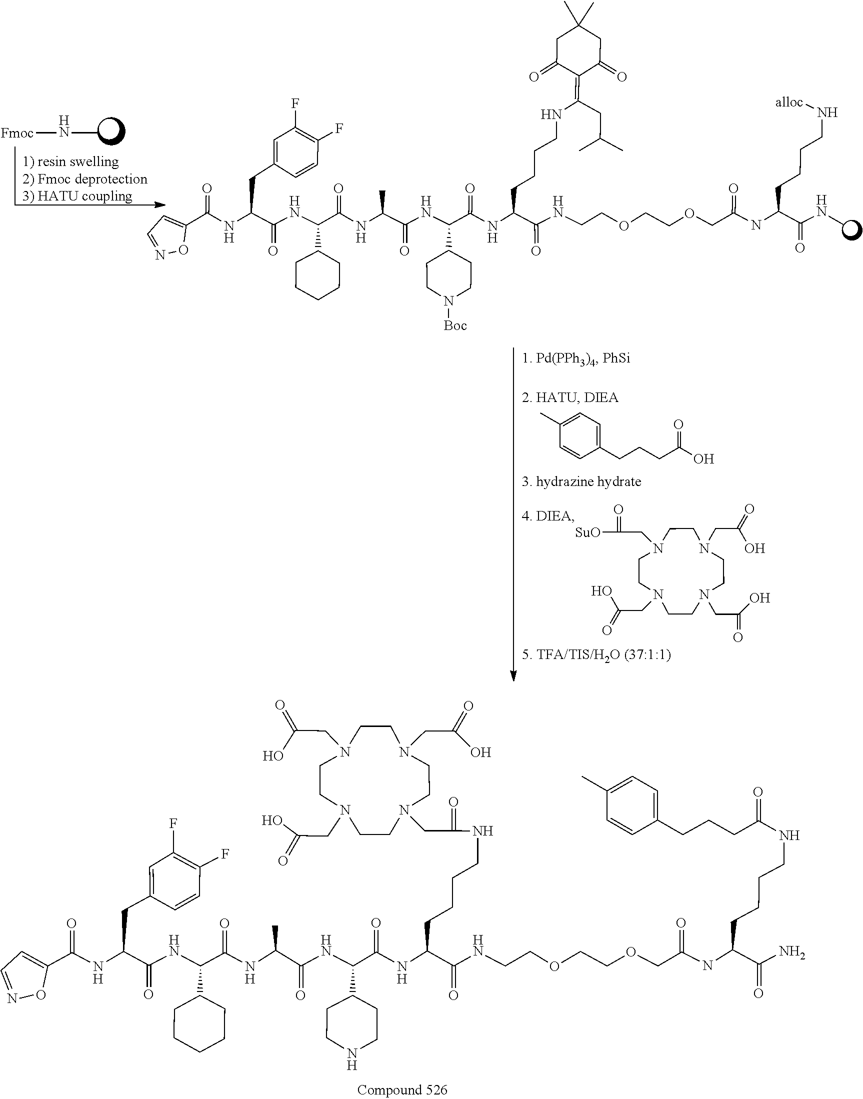

Steps 1-3: The 1^{st} intermediate on resin was prepared by standard Fmoc-based SPPS procedures from Sieber resin (General SPPS procedure 1-3).

Step 4: The resin from Step 3 (loading 0.53 mmol/g) was swollen with DCE (20 mL) for 0.5 h at room temperature. The resin was treated with Pd(PPh₃) 4 (367.59 mg, 0.318 mmol), phenylsilane (1376.90 mg, 12.720 mmol) and DCE (20 mL) for 1 h at room temperature. The resin was washed with a solution of 5% sodium (diethylcarbamothioyl) sulfanide trihydrate/NMP (5×20 mL). The resin was washed with 1% DIEA/NMP (5× 20 mL).

Step 5: The resin from Step 4 was swollen with NMP (20 mL) for 0.5 h at room temperature. The resin was treated with 20% piperidine (20 mL) for 0.5 h at room temperature. The resin was washed with NMP (6×20 mL). To the vessel were added 4-(4-methylphenyl) butanoic acid (226.6 mg, 1.27 mmol, 2 equiv), HATU (483.5 mg, 1.27 mmol), DIEA (328.7 mg, 2.54 mmol), and NMP (20 mL). The reactor was sealed and rotated strongly for 2 h at 30° C. The resulting mixture was washed with NMP (6×20 mL).

Step 6: The resin from Step 5 was swollen with NMP (20 mL) for 0.5 h at room temperature. A mixture of resin in 2% hydrazine hydrate/NMP (20 mL) was stirred at room temperature for 2 h. The resin was washed with NMP (8×20 mL).

Step 7: The resin from Step 6 was swollen with NMP (20 mL) for 0.5 h at room temperature. The resin was treated with 20% piperidine (20 mL) for 0.5 h at room temperature. The resin was washed with NMP (6×20 mL). The resin was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid (637.9 mg, 1.27 mmol, 2 equiv), DIEA (328.8 mg, 2.54 mmol), and NMP (20 mL) for 2 h at room temperature. The resin was washed with NMP (3× 20 mL).

Step 8: The product from Step 7 was cleaved from the resin by treatment of the resin with a solution of TFA/TIS/H₂O (v/v TFA/TIS/H₂O=37:1:1) (10 mL) for 1 h at 25° C. The mixture was diluted with cold ether, then the precipitate was centrifuged, to afford the crude product, which was purified by prep-HPLC to provide Compound 526 (27.3 mg, 7.5%) as a white solid. MS: Calc'd for C₇₆H₁₁₄F₂N₁₆O₁₉: 1592.84, found [M+2H]²⁺: 797.75.

### Synthesis of Compound 526-Lu (SEQ ID NO: 699)

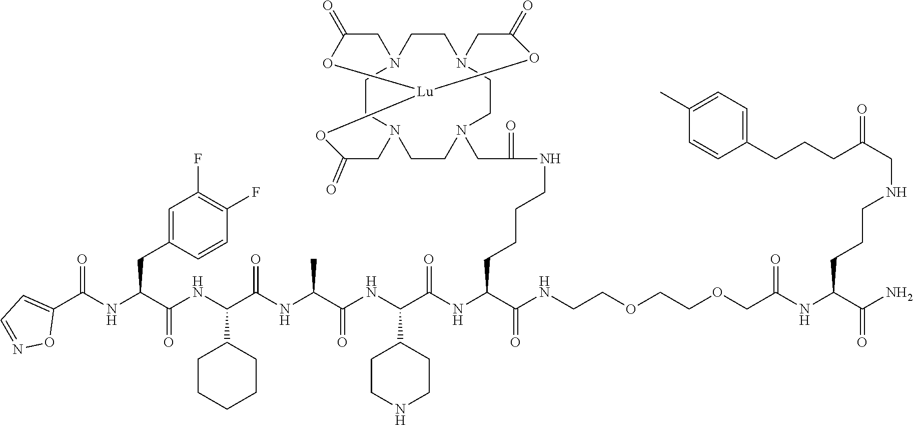

A solution of Compound 526 (20 mg, 0.012 mmol, 1.0 equiv) and lutetium (III) chloride (14.33 mg, 0.052 mmol, 4.4 equiv) in sodium acetate buffer (pH 5.6, 1.0 mL) was stirred at room temperature for 2 h. The crude mixture was purified by reversed-phase HPLC. The desired fractions were combined and lyophilized to afford Compound 526-Lu as a white solid (10.5 mg, yield 49.5%, 95.0% purity). MS: Calc'd for C₇₆H₁₁₁F₂LuN₁₆O₁₉: 1765.78, found [M+2H]²⁺: 883.8.

### Synthesis of Compound 526-In (SEQ ID NO: 700)

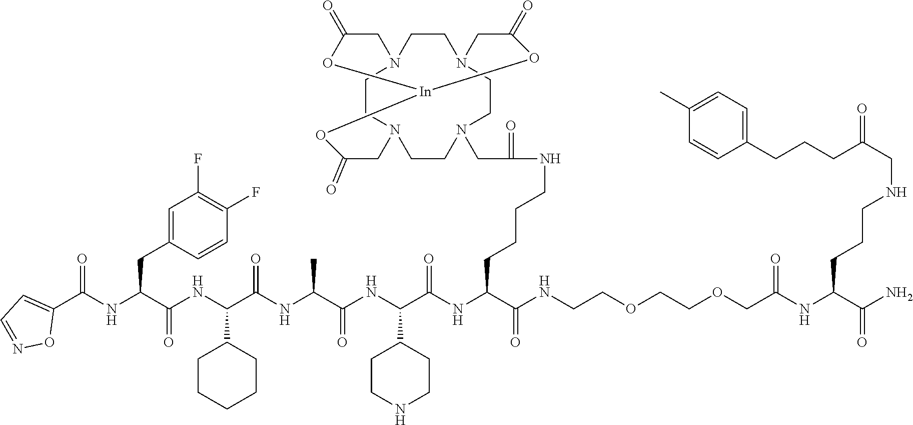

A solution of Compound 526 (35 mg, 0.020 mmol, 1.0 equiv) and indium (III) chloride (19.72 mg, 0.087 mmol, 4.4 equiv) in sodium acetate buffer (pH 5.6, 1.0 mL) was stirred at room temperature for 2 h. The crude mixture was purified by reversed-phase HPLC. The desired fractions were combined and lyophilized to afford Compound 526-In as a white solid (17.7 mg, yield 47.5%, 95.0% purity). MS: Calc'd for C₇₆H₁₁₁F₂InN₁₆O₁₉: 1704.72, found [M+2H]²⁺: 853.8.

### Synthesis of Compound 526-Ga (SEQ ID NO: 701)

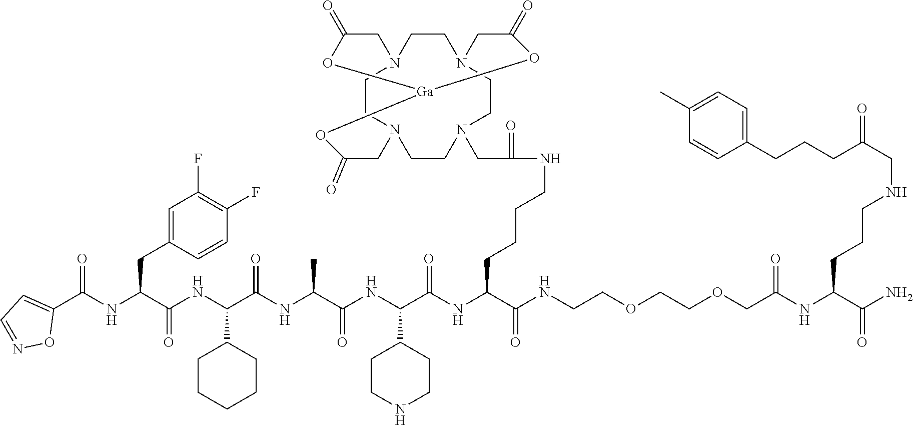

A solution of Compound 526 (20 mg, 0.012 mmol, 1 equiv) and gallium (III) chloride (8.97 mg, 0.052 mmol, 4.4 equiv) in sodium acetate buffer (pH 5.6, 1.0 mL) was stirred at room temperature for 12 h. The crude product was purified by reversed-phase HPLC. The desired fractions were combined and lyophilized to afford Compound 52-Ga as a white solid (9.6 mg, yield 48.0%, 96.6% purity). MS: Calc'd for C₇₆H₁₁₁F₂GaN₁₆O₁₉: 1658.74, found [M+2H]²⁺: 830.9.

### Example 527: (Compound 527) (SEQ ID NO: 682)

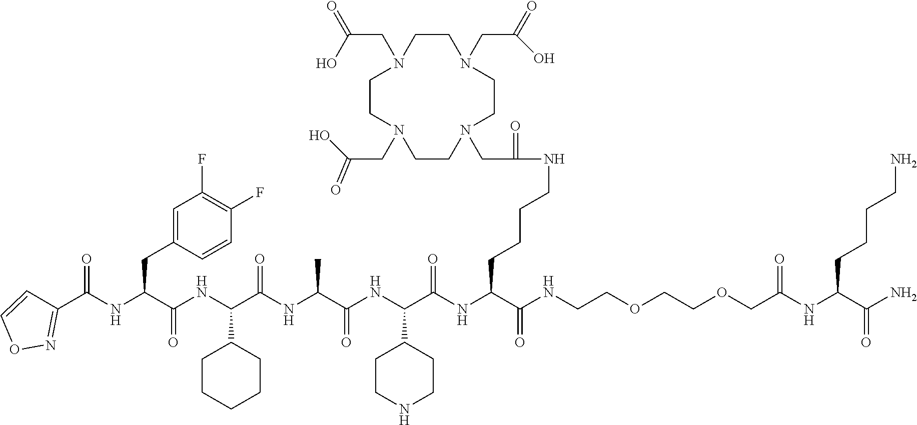 [SMILES: C[C@H](NC(=O)[C@@H](NC(=O)[C@H](Cc1ccc(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCNCC1]

MS: Calc'd for C₆₅H₁₀₂F₂N₁₆O₁₈: 1432.75, found [M+2H]²⁺: 717.75.

### Example 528: (Compound 528) (SEQ ID NO: 683)

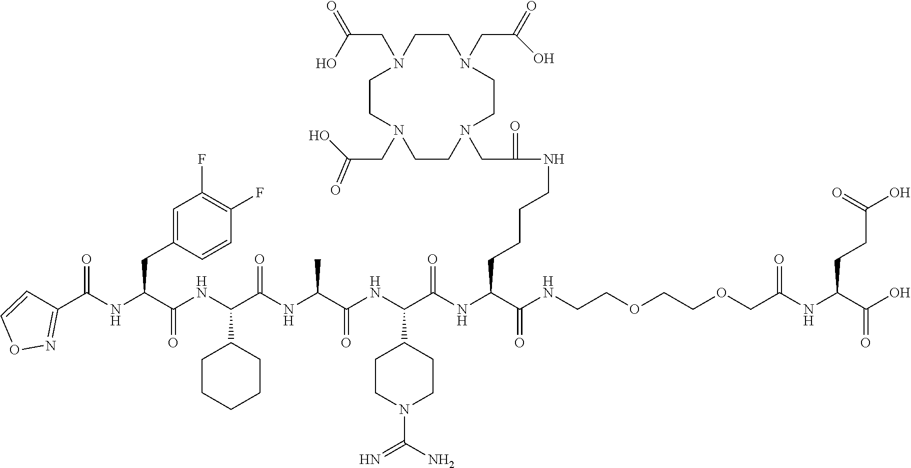 [SMILES: C[C@H](NC(=O)[C@@H](NC(=O)[C@H](Cc1ccc(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCC(=O)O)C(=O)O)C1CCN(C(=N)N)CC1]

MS: Calc'd for C₆₅H₉₈F₂N₁₆O₂₁: 1476.71, found [M+2H]²⁺: 739.6.

### Example 529: (Compound 529) (SEQ ID NO: 684)

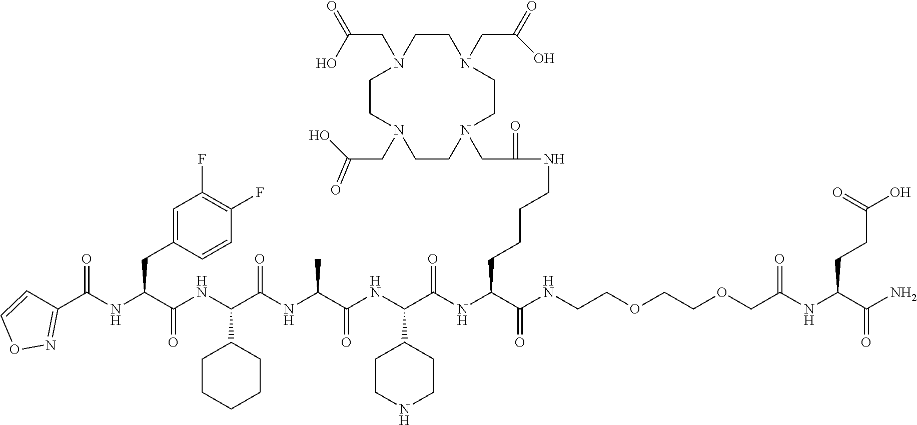 [SMILES: C[C@H](NC(=O)[C@@H](NC(=O)[C@H](Cc1ccc(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCC(=O)O)C(N)=O)C1CCNCC1]

MS: Calc'd for C₆₄H₉₇F₂N₁₅O₂₀: 1433.70, found [M+2H]²⁺: 718.1.

### Example 530: (Compound 530) (SEQ ID NO: 685)

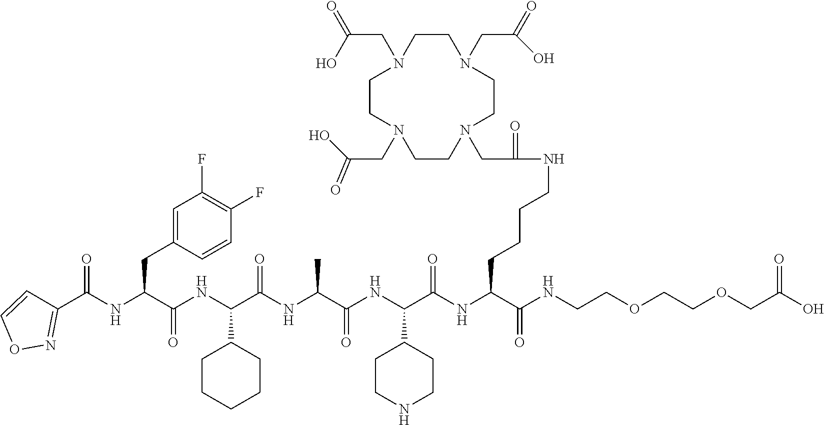 [SMILES: C[C@H](NC(=O)[C@@H](NC(=O)[C@H](Cc1ccc(F)c(F)c1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)O)C1CCNCC1]

MS: Calc'd for C₅₉H₈₉F₂N₁₃O₁₈: 1305.64, found [M+2H]²⁺: 654.1.

### Example 531: (Compound 531) (SEQ ID NO: 686)

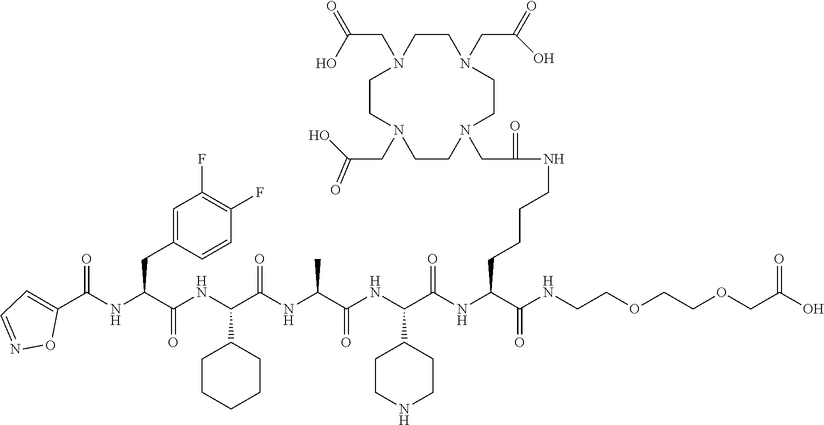 [SMILES: C[C@H](NC(=O)[C@@H](NC(=O)[C@H](Cc1ccc(F)c(F)c1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)O)C1CCNCC1]

MS: Calc'd for C₅₉H₈₉F₂N₁₃O₁₈: 1305.64, found [M+2H]²⁺: 654.15.

### Example 532: (Compound 532) (SEQ ID NO: 687)

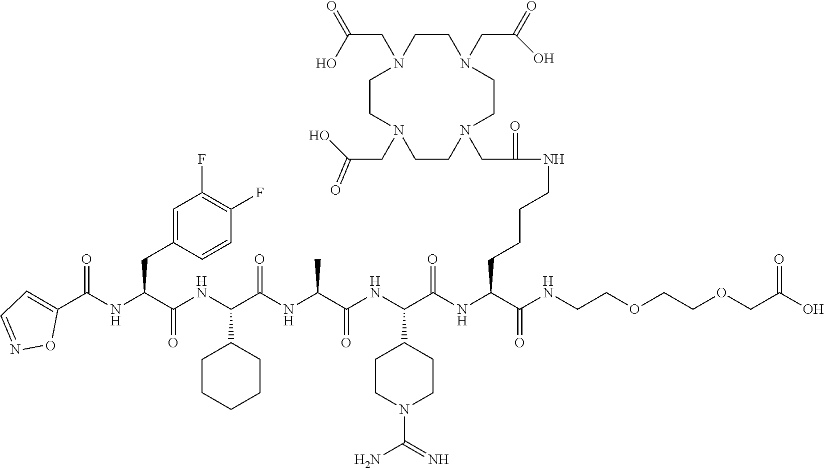 [SMILES: C[C@H](NC(=O)[C@@H](NC(=O)[C@H](Cc1ccc(F)c(F)c1)NC(=O)c1ccno1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)O)C1CCN(C(=N)N)CC1]

MS: Calc'd for C₆₀H₉₁F₂N₁₅O₁₈: 1347.66, found [M+2H]²⁺: 675.05.

### Example 533: (Compound 533) (SEQ ID NO: 688)

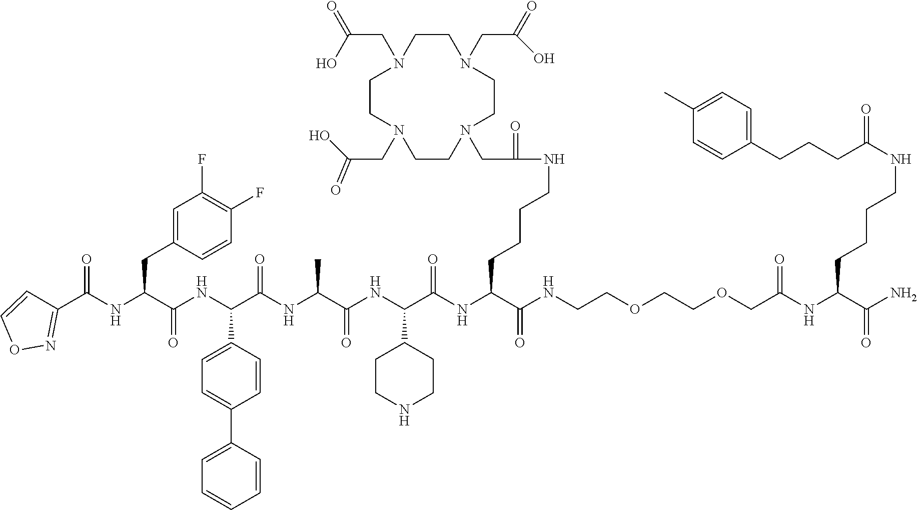 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)c2ccc(-c3ccccc3)cc2)C2CCNCC2)C(N)=O)cc1]

MS: Calc'd for C₈₂H₁₁₂F₂N₁₆O₁₉: 1662.83, found [M+2H]²⁺: 832.7.

### Example 534: (Compound 534) (SEQ ID NO: 689)

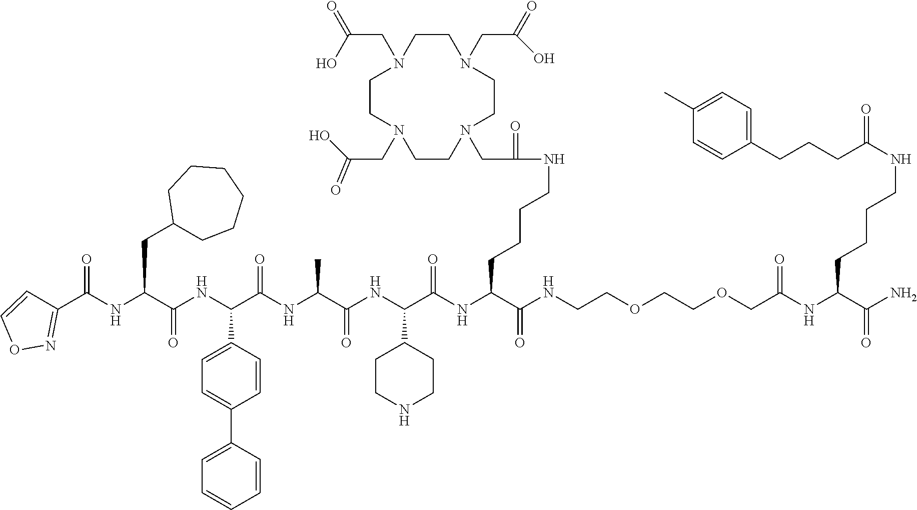 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](CC2CCCCCC2)NC(=O)c2ccon2)c2ccc(-c3ccccc3)cc2)C2CCNCC2)C(N)=O)cc1]

### Example 535: (Compound 535) (SEQ ID NO: 690)

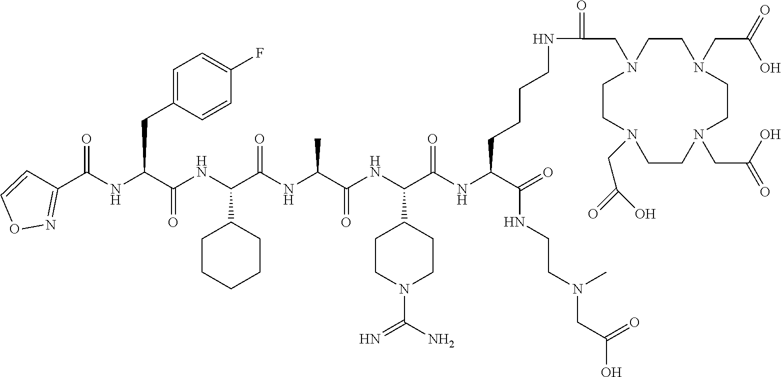 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCN(C)CC(=O)O)C1CCN(C(=N)N)CC1]

### Example 536: (Compound 536) (SEQ ID NO: 717)

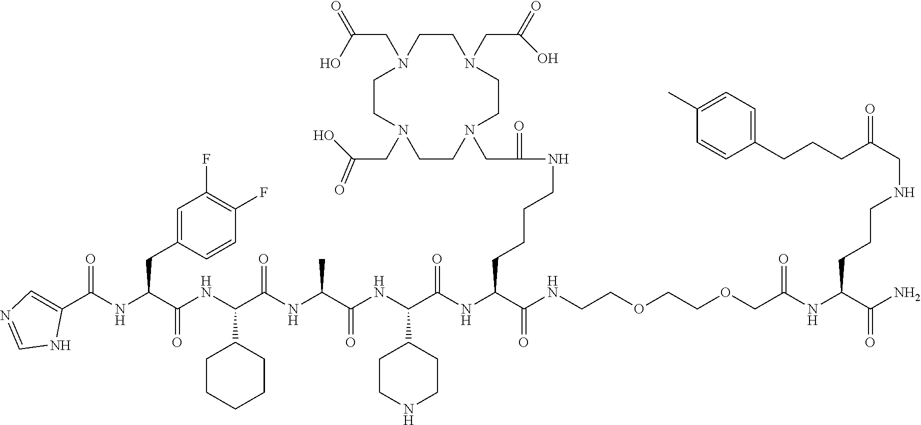 [SMILES: Cc1ccc(CCCC(=O)CNCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cnc[nH]2)C2CCCCC2)C2CCNCC2)C(N)=O)cc1]

MS: Calc'd for C₇₆H₁₁₅F₂N₁₇O₁₈: 1591.86, found [M+2H]²⁺: 797.5.

### Example 537: (Compound 537) (SEQ ID NO: 718)

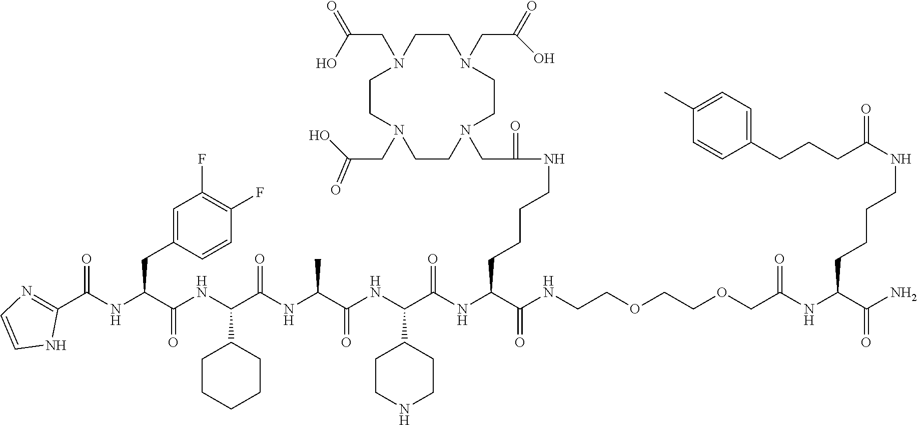 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ncc[nH]2)C2CCCCC2)C2CCNCC2)C(N)=O)cc1]

MS: Calc'd for C₇₆H₁₁₅F₂N₁₇O₁₈: 1591.86, found [M+2H]²⁺: 797.5.

### Example 538: (Compound 538) (SEQ ID NO: 719)

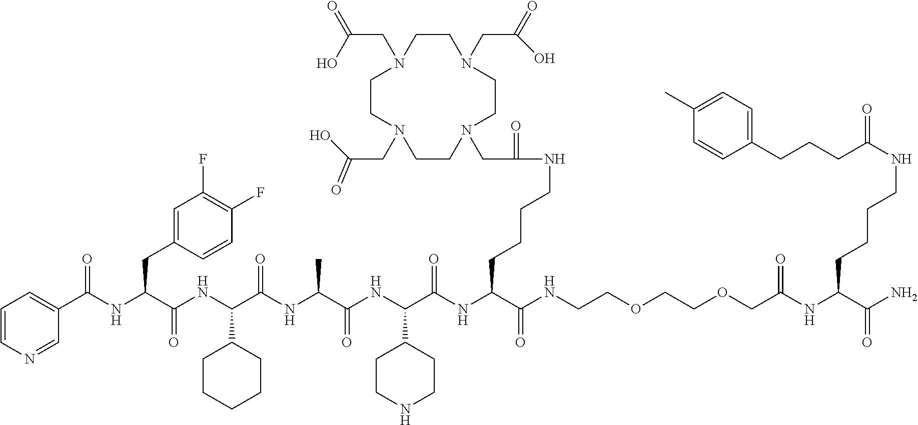 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cccnc2)C2CCCCC2)C2CCNCC2)C(N)=O)cc1]

MS: Calc'd for C₇₈H₁₆F₂N₁₆O₁₈: 1602.86, found [M+2H]²⁺: 802.9.

### Example 539: (Compound 539) (SEQ ID NO: 720)

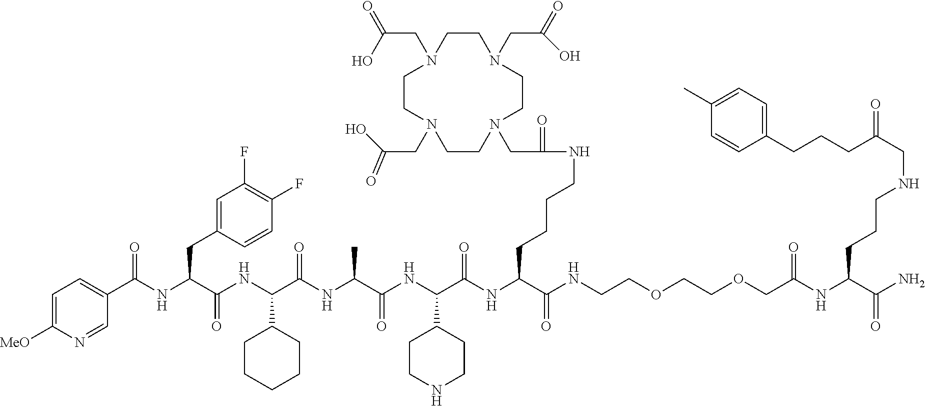 [SMILES: COc1ccc(C(=O)N[C@@H](Cc2ccc(F)c(F)c2)C(=O)N[C@H](C(=O)N[C@@H](C)C(=O)N[C@H](C(=O)N[C@@H](CCCCNC(=O)CN2CCN(CC(=O)O)CCN(CC(=O)O)CCN(CC(=O)O)CC2)C(=O)NCCOCCOCC(=O)N[C@@H](CCCNCC(=O)CCCc2ccc(C)cc2)C(N)=O)C2CCNCC2)C2CCCCC2)cn1]

MS: Calc'd for C₇₉H₁₁₈F₂N₁₆O₁₉: 1632.87, found [M+2H]²⁺: 818.0.

### Example 540: (Compound 540) (SEQ ID NO: 721)

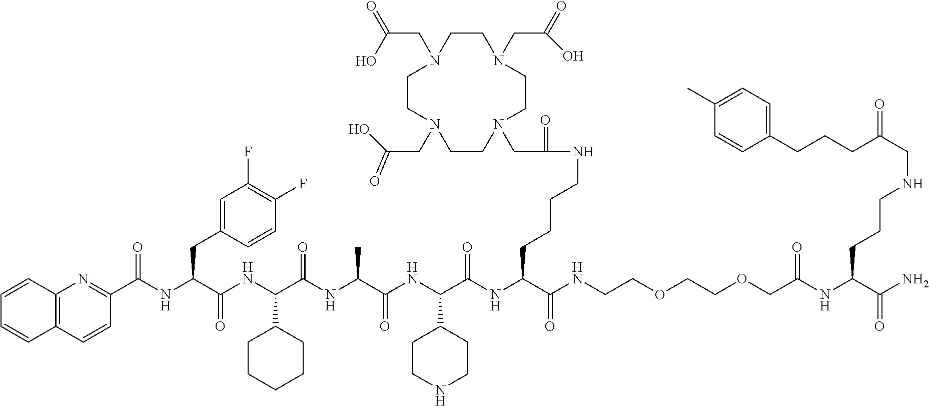 [SMILES: Cc1ccc(CCCC(=O)CNCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccc3ccccc3n2)C2CCCCC2)C2CCNCC2)C(N)=O)cc1]

MS: Calc'd for C₈₂H₁₁₈F₂N₁₆O₁₈: 1652.88, found [M+2H]²⁺: 828.0.

### Example 541: (Compound 541) (SEQ ID NO: 722)

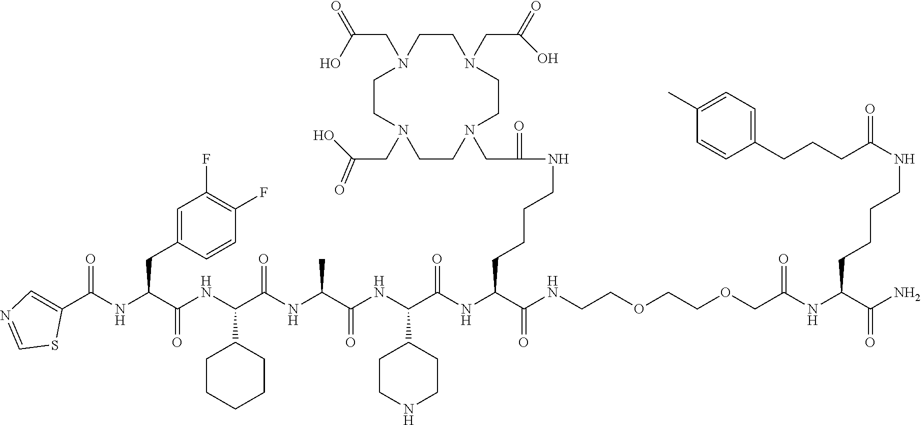 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cncs2)C2CCCCC2)C2CCNCC2)C(N)=O)cc1]

MS: Calc'd for C₇₆H₁₁₄F₂N₁₆O₁₈S: 1608.82, found [M+2H]²⁺: 805.9.

### Example 542: (Compound 542) (SEQ ID NO: 723)

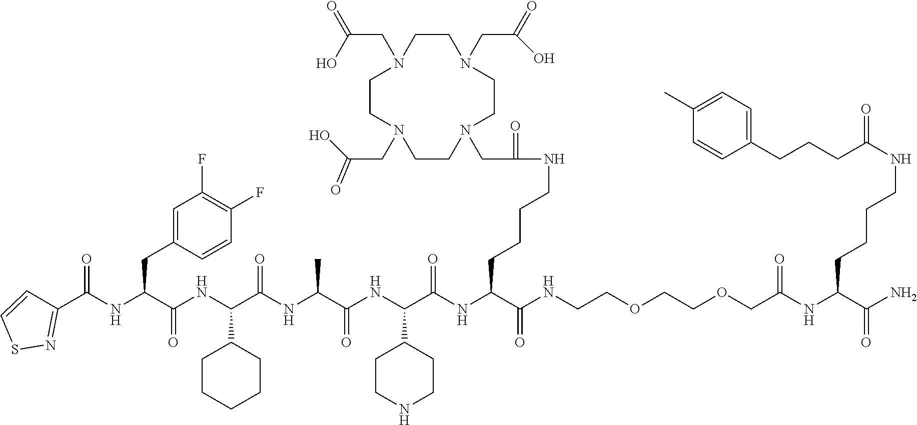 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccsn2)C2CCCCC2)C2CCNCC2)C(N)=O)cc1]

MS: Calc'd for C₇₆H₁₁₄F₂N₁₆O₁₈S: 1655.93, found [M+2H]²⁺: 805.9.

### Example 543: (Compound 543) (SEQ ID NO: 724)

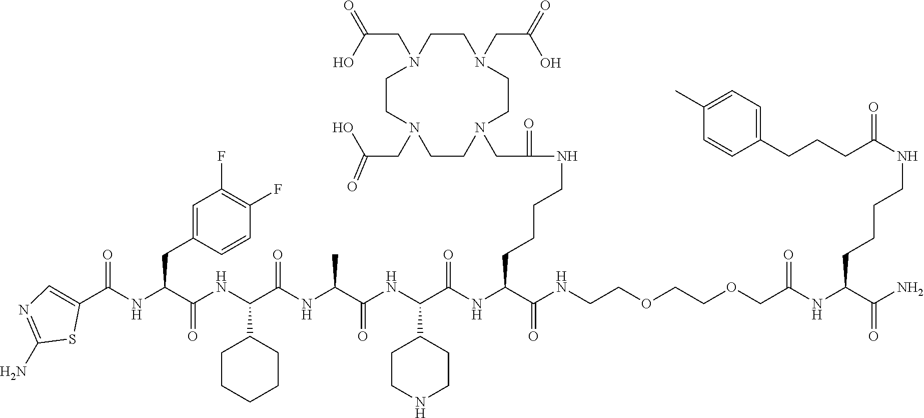 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cnc(N)s2)C2CCCCC2)C2CCNCC2)C(N)=O)cc1]

MS: Calc'd for C₇₆H₁₁₅F₂N17018S: 1623.83, found [M+2H]²⁺: 813.5.

### Example A-1: Parenteral Pharmaceutical Composition

To prepare a parenteral pharmaceutical composition suitable for administration by injection (subcutaneous, intravenous), 0.001-500 mg of a compound Formula (I), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### BIOLOGY EXAMPLES

### Example B-1: Human PAR2 Binding Assay

### Membrane Preparation

Crude membrane fractions are prepared from T-Rex-293 (Thermo Fisher) cells endogenously expressing the human protease activated receptor 2 (PAR2). The cells are grown to 85-100% confluence on standard tissue culture dishes in DMEM (Fisher Scientific) supplemented with 10% FBS (Gemini) and 24 hours prior to membrane prepping the cells are treated with 1 μg/ml tetracycline (Sigma-Aldrich) to induce receptor expression. To prepare membranes, cells are scraped and collected in 1× Dulbecco's phosphate buffered saline (Corning) and then pelleted at 1000 RPM's. The cell pellet is reconstituted in membrane preparation buffer (20 mM HEPES, 6 mM MgCl₂ and 1 mM EGTA, protease inhibitor tablets (Pierce), pH 7.4) and placed in a cell disruption vessel (Parr Instrument company) at 1000 PSI for 30 minutes on ice. The pressurized contents are then released and spun down at 1000 RPMs and the supernatant is collected and further centrifuged at 15,000 RPMs to pellet the membranes. The membrane pellet is resuspended in membrane preparation buffer, snap freezed and stored at −80° C. for later use.

### PAR2 Binding Assay Protocol:

The human PAR2 membrane binding assay utilizes the following components: radiolabel [3H]-furoyl-LIGRL-NH₂ (Pharmaron), crude T-Rex-293 human PAR2 membranes, and competing small molecule and peptide ligands. The assay is initiated by combining in assay buffer (50 mM Tris HCl, 10 mM MgCl₂, 1 mM EGTA, 0.1% BSA, pH 7.4) a dose response of competing ligand (the final concentrations are typically 0-10,000 nM), T-Rex-293 human PAR2 membranes, and [3H]-furoyl-LIGRL-NH₂ at a final concentration of 2.5 nM in a 96-well assay plate and allowed to incubate for 240 minutes at room temperature. Assay contents are then filtered through unifilter GF/C microplates (Revvity) and washed with 4×400 μl of cold wash buffer (50 mM Tris HCl, pH 7.4). Assay plates are read using a MicroBeta² (Revvity) and Ki values for compounds are determined using a GraphPad Prism 10 non-linear regression analysis.

Illustrative biological activity of compounds is demonstrated in Table G. The metal complexes in Table G comprise nonradioactive gallium, indium, and lutetium.

TABLE G
Representative Binding Activity of PAR2 Ligands and Conjugates

| | hPAR2 Binding |
|---|---|
| Compound | Ki* (90 min, nM) |
| 1 | A |
| 2 | A |
| 3 | A |
| 4 | A |
| 5 | A |
| 6 | A |
| 7 | A |
| 8 | A |
| 9 | C |
| 10 | A |
| 12 | A |
| 14 | A |
| 15 | A |
| 16 | C |
| 17 | A |
| 18 | A |
| 20 | A |
| 21 | A |
| 22 | A |
| 23 | A |
| 24 | A |
| 25 | A |
| 26 | A |
| 27 | C |
| 28 | A |
| 29 | A |
| 30 | A |
| 31 | A |
| 32 | A |
| 33 | A |
| 34 | A |
| 35 | C |
| 36 | A |
| 37 | A |
| 38 | A |
| 39 | A |
| 40 | A |
| 41 | C |
| 48 | A |
| 49 | A |
| 50 | A |
| 51 | A |
| 52 | A |
| 53 | C |
| 54 | C |
| 55 | A |
| 56 | A |
| 57 | A |
| 58 | A |
| 59 | A |
| 61 | A |
| 62 | A |
| 63 | A |
| 66 | A |
| 67 | A |
| 68 | A |
| 72 | A |
| 74 | A |
| 75 | A |
| 76 | A |
| 77 | A |
| 78 | C |
| 79 | C |
| 85 | A |
| 86 | A |
| 87 | A |
| 91 | A |
| 92 | A |
| 93 | A |
| 94 | A |
| 95 | A |
| 96 | A |
| 97 | A |
| 98 | A |
| 99 | A |
| 100 | C |
| 101 | A |
| 102 | A |
| 103 | A |
| 104 | A |
| 105 | A |
| 106 | A |
| 107 | A |
| 108 | A |
| 109 | B |
| 110 | A |
| 111 | A |
| 112 | A |
| 113 | A |
| 114 | A |
| 115 | A |
| 116 | A |
| 117 | A |
| 118 | A |
| 119 | A |
| 120 | A |
| 121 | A |
| 122 | A |
| 123 | A |
| 124 | A |
| 125 | A |
| 126 | A |
| 127 | C |
| 128 | C |
| 129 | C |
| 130 | B |
| 133 | A |
| 134 | B |
| 135 | A |
| 136 | B |
| 137 | A |
| 138 | A |
| 141 | B |
| 142 | B |
| 143 | A |
| 144 | A |
| 145 | A |
| 146 | A |
| 147 | A |
| 148 | A |
| 149 | C |
| 150 | A |
| 151 | A |
| 152 | A |
| 153 | C |
| 154 | A |
| 155 | C |
| 156 | A |
| 157 | A |
| 158 | A |
| 159 | A |
| 160 | B |
| 161 | C |
| 162 | A |
| 165 | A |
| 166 | A |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 178 | A |
| 181 | A |
| 183 | A |
| 184 | A |
| 185 | A |
| 186 | A |
| 187 | B |
| 189 | B |
| 190 | A |
| 191 | A |
| 192 | A |
| 193 | A |
| 194 | A |
| 195 | A |
| 196 | A |
| 198 | A |
| 199 | A |
| 200 | A |
| 201 | A |
| 202 | A |
| 203 | A |
| 204 | C |
| 206 | A |
| 207 | A |
| 208 | A |
| 209 | A |
| 210 | A |
| 219 | A |
| 220 | A |
| 221 | A |
| 222 | A |
| 223 | A |
| 224 | A |
| 225 | A |
| 226 | A |
| 227 | A |
| 228 | A |
| 229 | A |
| 230 | B |
| 231 | A |
| 232 | A |
| 233 | A |
| 234 | A |
| 235 | B |
| 236 | B |
| 237 | A |
| 238 | A |
| 238-In | A |
| 239 | A |
| 240 | A |
| 240-In | A |
| 240-Lu | A |
| 240-Ga | A |
| 241 | A |
| 242 | A |
| 243 | A |
| 244 | A |
| 245 | A |
| 246 | A |
| 247 | A |
| 249 | A |
| 250 | A |
| 250-Ga | A |
| 250-Lu | A |
| 250-In | A |
| 251 | A |
| 252 | A |
| 253 | A |
| 254 | A |
| 255 | A |
| 256 | A |
| 257 | A |
| 258 | A |
| 259 | A |
| 260 | A |
| 261 | A |
| 262 | A |
| 263 | A |
| 264 | A |
| 265 | A |
| 266 | A |
| 267 | B |
| 268 | C |
| 269 | A |
| 270 | A |
| 271 | A |
| 272 | A |
| 273 | A |
| 274 | A |
| 275 | A |
| 276 | A |
| 278 | A |
| 279 | A |
| 280 | A |
| 281 | A |
| 282 | A |
| 283 | A |
| 284 | A |
| 285 | A |
| 286 | A |
| 287 | A |
| 289 | A |
| 290 | A |
| 291 | A |
| 292 | A |
| 293 | A |
| 294 | A |
| 295 | A |
| 297 | A |
| 298 | A |
| 299 | A |
| 300 | A |
| 301 | A |
| 302 | A |
| 303 | A |
| 304 | A |
| 305 | A |
| 306 | A |
| 307 | A |
| 308 | A |
| 309 | A |
| 310 | A |
| 311 | A |
| 312 | A |
| 313 | A |
| 314 | B |
| 315 | A |
| 316 | A |
| 319 | A |
| 320 | A |
| 321 | A |
| 322 | A |
| 323 | A |
| 324 | A |
| 325 | A |
| 326 | A |
| 327 | A |
| 328 | A |
| 329 | A |
| 330 | A |
| 331 | A |
| 332 | A |
| 333 | A |
| 334 | A |
| 335 | A |
| 336 | A |
| 337 | A |
| 339 | A |
| 340 | A |
| 341 | A |
| 342 | A |
| 344 | A |
| 345 | A |
| 346 | A |
| 347 | A |
| 348 | A |
| 349 | A |
| 350 | A |
| 351 | A |
| 352 | A |
| 354 | A |
| 355 | A |
| 356 | A |
| 357 | A |
| 358 | A |
| 359 | A |
| 360 | A |
| 361 | A |
| 362 | A |
| 363 | A |
| 364 | A |
| 365 | A |
| 366 | A |
| 367 | A |
| 368 | A |
| 369 | A |
| 370 | A |
| 371 | A |
| 372 | A |
| 374 | A |
| 375 | A |
| 376 | A |
| 377 | A |
| 378 | A |
| 379 | A |
| 380 | A |
| 381 | A |
| 382 | A |
| 383 | A |
| 384 | A |
| 385 | A |
| 386 | A |
| 387 | A |
| 388 | A |
| 391 | A |
| 392 | A |
| 393 | A |
| 394 | A |
| 395 | A |
| 396 | A |
| 397 | A |
| 398 | A |
| 399 | A |
| 400 | A |
| 401 | A |
| 402 | A |
| 402-In | A |
| 403 | A |
| 404 | A |
| 405 | A |
| 408 | A |
| 409 | A |
| 410 | A |
| 411 | A |
| 412 | A |
| 415 | A |
| 416 | A |
| 417 | A |
| 418 | A |
| 419 | A |
| 420 | A |
| 421 | A |
| 421-In | A |
| 422 | A |
| 423 | A |
| 424 | A |
| 425 | A |
| 426 | A |
| 427 | A |
| 428 | A |
| 429 | A |
| 430 | A |
| 431 | A |
| 432 | A |
| 433 | A |
| 434 | A |
| 435 | A |
| 436 | A |
| 437 | A |
| 438 | A |
| 439 | A |
| 440 | A |
| 441 | A |
| 442 | A |
| 443 | A |
| 444 | A |
| 445 | A |
| 446 | A |
| 447 | A |
| 448 | A |
| 449 | A |
| 450 | A |
| 452 | A |
| 453 | A |
| 454 | A |
| 455 | A |
| 456 | A |
| 457 | A |
| 458 | A |
| 459 | A |
| 460A | A |
| 460B | A |
| 461 | A |
| 466 | A |
| 467 | A |
| 468 | A |
| 469 | A |
| 470 | A |
| 471 | A |
| 472 | A |
| 473 | A |
| 474 | A |
| 475 | A |
| 477 | A |
| 478 | A |
| 480 | A |
| 481 | A |
| 484 | A |
| 485 | A |
| 486 | A |
| 488 | A |
| 489 | A |
| 490 | A |
| 491 | A |
| 493 | A |
| 494 | A |
| 495 | A |
| 497 | A |
| 498 | A |
| 504A | C |
| 504B | C |
| 505 | A |
| 518 | A |
| 519 | A |
| 520 | A |
| 521 | A |
| 522 | A |
| 523 | A |
| 524 | A |
| 525 | A |
| 526 | A |
| 526-In | A |
| 526-Lu | A |
| 526-Ga | A |
| 527 | A |
| 528 | A |
| 529 | A |
| 530 | A |
| 531 | A |
| 532 | A |
| 533 | A |
| 536 | A |
| 537 | A |
| 539 | A |
| 540 | A |
| 541 | A |
| 542 | A |
| 543 | A |

*A is <100 nM; B is 100-250 nM; C is >250 nM

### Example B-2: Biodistribution of ¹¹¹In[In] Complex of Compound 240 in Mouse Tumor Model

TABLE H
Study outline

| | N | Dose | Dose | Time |
|---|---|---|---|---|
| Treatment | value | (MBq/Mouse) | (nmol/mouse) | (h) |
| ¹¹¹In[In]- | 4 | 5-7 | 1 | 0.5 |
| Compound 240 | | | | |
| ¹¹¹In[In]- | 4 | 5-7 | 1 | 2 |
| Compound 240 | | | | |
| ¹¹¹In[In]- | 4 | 5-7 | 1 | 5 |
| Compound 240 | | | | |
| ¹¹¹In[In]- | 4 | 5-7 | 1 | 24 |
| Compound 240 | | | | |
| ¹¹¹In[In]- | 4 | 5-7 | 1 | 72 |
| Compound 240 | | | | |
| ¹¹¹In[In]- | 4 | 5-7 | 1 + | 2 |
| Compound 240 + | | 0 | 100 nm | |
| ¹¹⁵In[In] | | | | |
| Compound 240 | | | | |

*Treatment schedule is Q1D × 1(IV)

Study Details: 24 h prior to the start of the biodistribution Compound 240 was radiolabeled.

Prior to initiation of the biodistribution study, female Swiss nude mice were subcutaneously inoculated with 10×10⁶ human cancer cells.

When tumors were between 150-300 mm³, animals were randomized and received a single IV injection of 200 μL into the caudal vein via a catheter with 5-7 MBq of ¹¹¹In[In] Compound 240 (1 nmol) per animal. For the competition study arm, 1 nmol of ¹¹¹In[In]-Compound 240 was co injected with 100 nmol of ¹¹⁵In[In]-Compound 240.

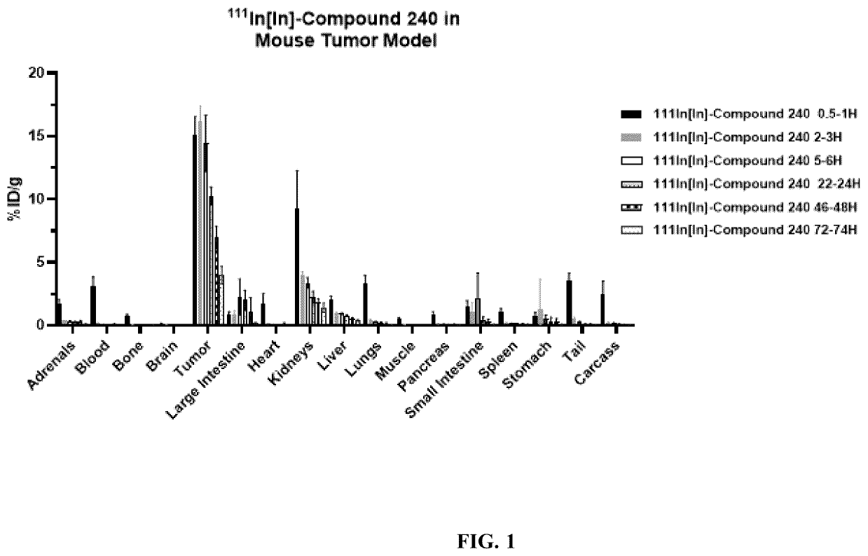

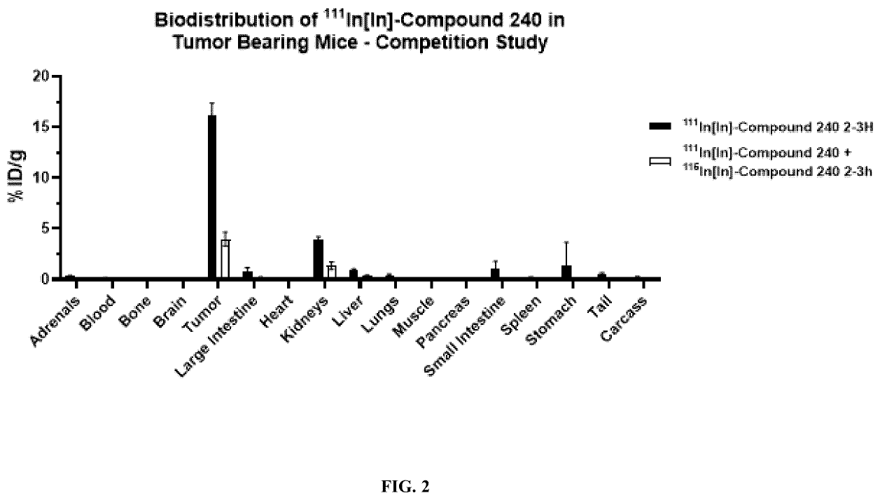

After drug administration, animals were euthanized at timepoints (0.5 h, 2 h, 5h, 24 h and 72h) and organs (blood, heart, muscle, lungs, intestines, spleen, bone, brain, tail, carcass, kidneys, liver and tumor) were collected, weighed, and ÿ radioactivity assessed in each organ/tissue. Activity was quantitated and expressed as % ID/g (Percentage of Initial Dose/gram of tissue). See FIG. 1 and FIG. 2.

### Example B-3: Biodistribution of ¹¹¹In[In] Complex of Compound 250 in Mouse Tumor Model

TABLE K
Study outline

| | N | Dose | Dose | Time |
|---|---|---|---|---|
| Treatment | value | (MBq/Mouse) | (nmol/mouse) | (h) |
| ¹¹¹In[In]- | 4 | 5-7 | 1 | 0.5 |
| Compound 250 | | | | |
| ¹¹¹In[In]- | 4 | 5-7 | 1 | 2 |
| Compound 250 | | | | |
| ¹¹¹In[In]- | 4 | 5-7 | 1 | 5 |
| Compound 250 | | | | |
| ¹¹¹In[In]- | 4 | 5-7 | 1 | 24 |
| Compound 250 | | | | |
| ¹¹¹In[In]- | 4 | 5-7 | 1 | 72 |
| Compound 250 | | | | |
| ¹¹¹In[In]- | 4 | 5-7 | 1 + | 2 |
| Compound 250 + | | 0 | 100 nm | |
| ¹¹⁵In[In]- | | | | |
| Compound 250 | | | | |

*Treatment schedule is Q1D × 1(IV)

Study Details: 24 h prior to the start of the biodistribution Compound 250 was radiolabeled.

Prior to initiation of the biodistribution study, female Swiss nude mice were subcutaneously inoculated with 10×10⁶ human cancer cells.

When tumors were between 150-300 mm³, animals were randomized and received a single IV injection of 200 μL into the caudal vein via a catheter with 5-7 MBq of ¹¹¹In[In] Compound 250 (1 nMol) per animal. For the competition study arm, 1 nMol of ¹¹¹In[In]-Compound 250 was co injected with 100 nMol of ¹¹⁵In[In]-Compound 250.

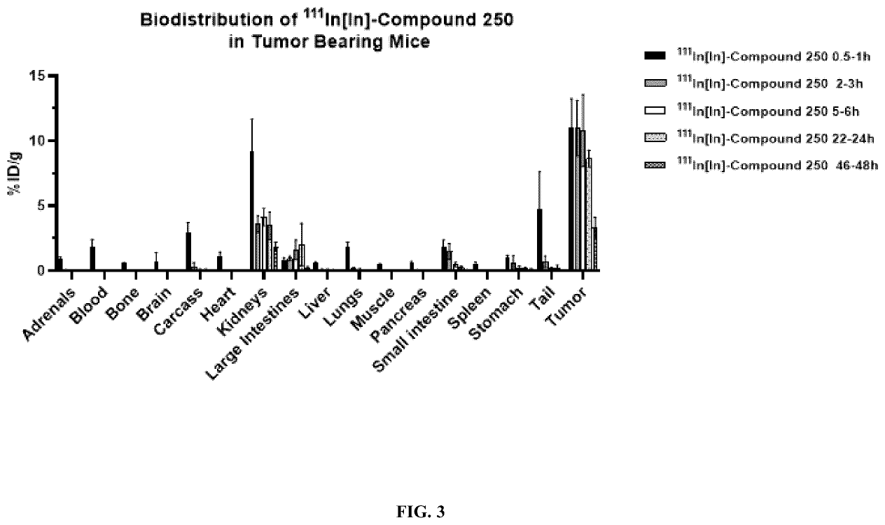

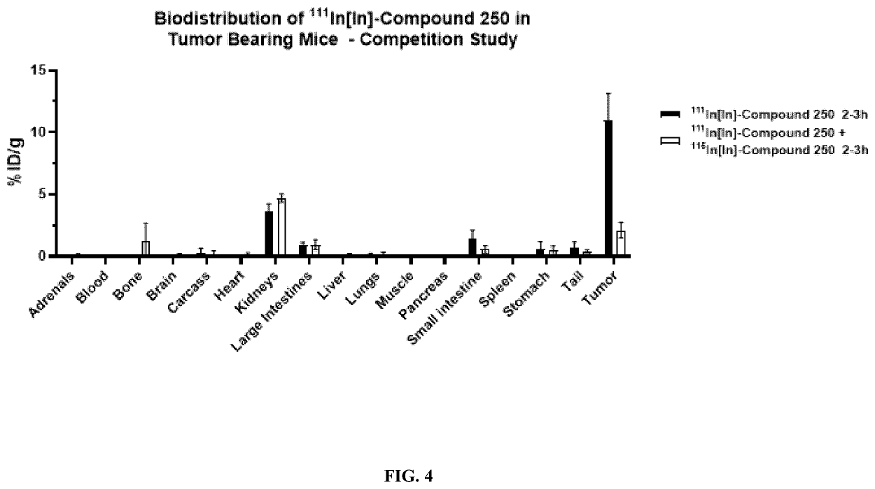

After drug administration, animals were euthanized at timepoints (0.5 h, 2 h, 5h, 24 h and 72h) and organs (blood, heart, muscle, lungs, intestines, spleen, bone, brain, tail, carcass, kidneys, liver and tumor) were collected, weighed, and ÿ radioactivity assessed in each organ/tissue. Activity was quantitated and expressed as % ID/g (Percentage of Initial Dose/gram of tissue). See FIG. 3 and FIG. 4.

### Example B-4: Biodistribution of ¹¹¹In[In]-Compound 402 in Mouse Tumor Model

TABLE M
Study Outline

| | N | Dose | Dose | Time |
|---|---|---|---|---|
| Treatment | value | (MBq/Mouse) | (nmol/mouse) | (h) |
| ¹¹¹In[In]- | 4 | 5-7 | 1 | 0.5 |
| Compound 402 | | | | |
| ¹¹¹In[In]- | 4 | 5-7 | 1 | 2-3 |
| Compound 402 | | | | |
| ¹¹¹In[In]- | 4 | 5-7 | 1 | 5-6 |
| Compound 402 | | | | |
| ¹¹¹In[In]- | 4 | 5-7 | 1 | 23-24 |
| Compound 402 | | | | |
| ¹¹¹In[In]- | 4 | 5-7 | 1 | 72-74 |
| Compound 402 | | | | |
| ¹¹¹In[In]- | 4 | 5-7 | 1 + | 2-3 h |
| Compound 402 + | | 0 | 100 nm | |
| ¹¹⁵In[In]- | | | | |
| Compound 402 | | | | |

*Treatment schedule is Q1D × 1(IV)

24 h prior to the start of the biodistribution Compound 402 was radiolabeled

Prior to initiation of the biodistribution study, female Swiss nude mice were subcutaneously inoculated with 10×10⁶ human cancer cells.

When tumors were between 150-300 mm³, animals were randomized and received a single IV injection of 200 μL into the caudal vein via a catheter with 5-7 MBq of ¹¹¹In[In] Compound 402 (1 nMol) per animal. For the competition study arm, 1 nMol of ¹¹¹In[In]-Compound 402 was co injected with 100 nMol of ¹¹⁵In[In]-Compound 402.

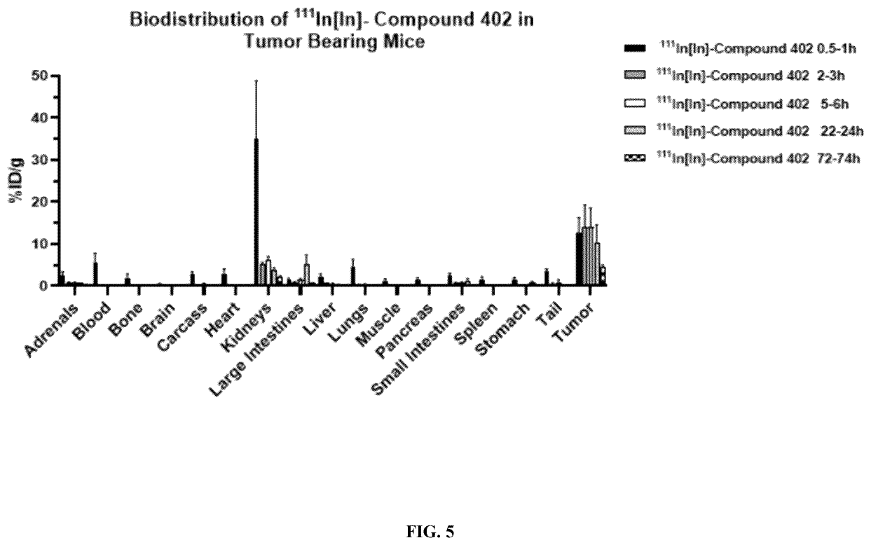

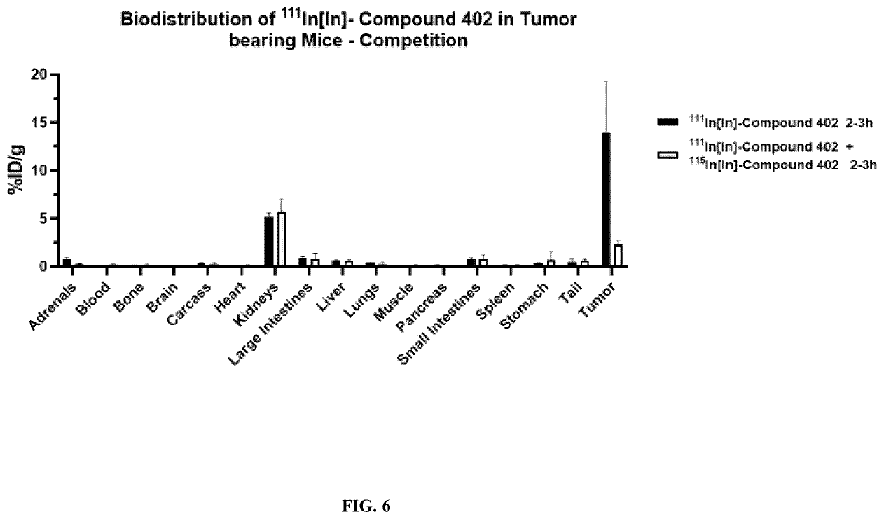

After drug administration, animals were euthanized at timepoints (0.5 h, 2 h, 5h, 24 h and 72h) and organs (Blood, Heart, Muscle, Lungs, Intestines, Spleen, Bone, Brain, Tail, Carcass, Kidneys, Liver and Tumor) were collected, weighed, and ÿ radioactivity assessed in each organ/tissue. Activity was quantitated and expressed as % ID/g (Percentage of Initial Dose/gram of tissue). See FIG. 5 and FIG. 6.

### Example B-5: Biodistribution of ¹⁷⁷Lu[Lu]-Compound 526 in Mouse Tumor Model

TABLE O
Study Outline

| | N | Dose | Dose | Time |
|---|---|---|---|---|
| Treatment | value | (MBq/Mouse) | (nmol/mouse) | (h) |
| ¹⁷⁷Lu[Lu]- | 4 | 5-7 | 1 | 0.5 |
| Compound 526 | | | | |
| ¹⁷⁷Lu[Lu]- | 4 | 5-7 | 1 | 2 |
| Compound 526 | | | | |
| ¹⁷⁷Lu[Lu]- | 4 | 5-7 | 1 | 5 |
| Compound 526 | | | | |
| ¹⁷⁷Lu[Lu]- | 4 | 5-7 | 1 | 24 |
| Compound 526 | | | | |
| ¹⁷⁷Lu[Lu]- | 4 | 5-7 | 1 | 72 |
| Compound 526 | | | | |
| ¹⁷⁷Lu[Lu]- | 4 | 5-7 | 1 | 168 |
| Compound 526 | | | | |
| ¹⁷⁷Lu[Lu]- | 4 | 5-7 | 1 + | 3 h |
| Compound 526 + | | 0 | 100 nm | |
| ¹⁷⁵In[In] | | | | |
| Compound 526 | | | | |

*Treatment schedule is Q1D × 1(IV)

24 h prior to the start of the biodistribution Compound 526 was radiolabeled.

Prior to initiation of the biodistribution study, female Swiss nude mice were subcutaneously inoculated with 10×10⁶ human cancer cells.

When tumors were between 150-300 mm³, animals were randomized and received a single IV injection of 200 μL into the caudal vein via a catheter with 5-7 MBq of ¹⁷⁷Lu[Lu] Compound 526 (1 nMol) per animal. For the competition study arm, 1 nMol of ¹⁷⁷Lu[Lu]-Compound 526 was co injected with 100 nMol of ¹⁷⁵Lu[Lu]-Compound 526.

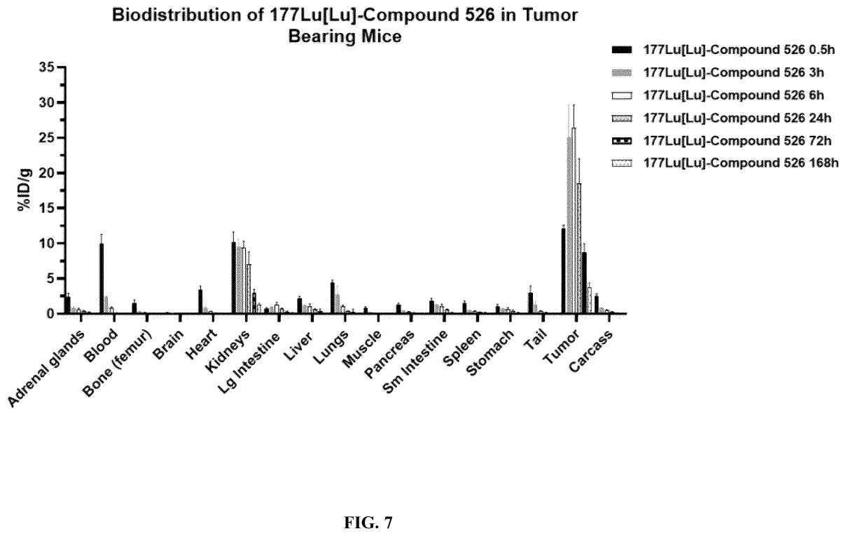

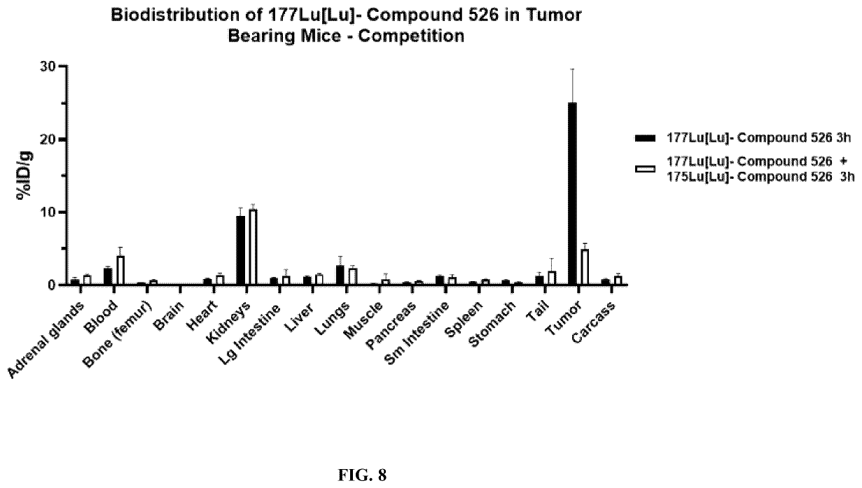

After drug administration, animals were euthanized at timepoints (0.5 h, 3 h, 5h, 24 h and 72 h and 168h) and organs (Blood, Heart, Muscle, Lungs, Intestines, Spleen, Bone, Brain, Tail, Carcass, Kidneys, Liver and Tumor) were collected, weighed, and ÿ radioactivity assessed in each organ/tissue. Activity was quantitated and expressed as % ID/g (Percentage of Initial Dose/gram of tissue). See FIG. 7 and FIG. 8.

### Example B-6: Efficacy Model

Tumor cells expressing hPAR2 are inoculated subcutaneously in the right rear flank by injection of 10 million (1×10⁷) cells in 200 μL of RPMI 1640 (without phenol red) of 91 female Swiss nude mice. Tumor cell implantation is performed 24 to 72 hours after a whole-body irradiation with a gamma-source (2 Gy (Nude mice).

Once tumors reach a mean value of approximately 225 mm³, animals are randomized into 7 groups (9 animals per group) and dosed with vehicle, 90, 45, or 15 MBq of ¹⁷⁷Lu-radiolabeled complexes (at 1.0 nmol of each compound). Tumors are measured twice weekly and tumor volume are determined by the formula: Tumor Volume=(width\hat{ }(2)×length)/2. Mouse mass is calculated 2-3 times per week. Tumor growth inhibition (TGI mean %) will be calculated as follows: TGI (%)=(1−(mean TV on treated group)/(mean TV on control group))×100. TGI % will be calculated up to the last day on which the number of animals in the vehicle group (control) exceeds 50%.

### Example B-7: Safety and Biodistribution Study in Patients with Gastric Cancer, Colorectal Cancer, Pancreatic Cancer, or Solid Tumor Malignancies

A non-limiting example of a phase 1 safety and dosimetry study of ⁶⁸Ga or ¹⁷⁷Lu-complex of a compound of Formula (I) in patients with gastric, colorectal, pancreatic, and solid tumor malignancies of any anatomical origin as described below.

This is an open-label, first-in-human, Phase 1 study of ⁶⁸Ga and ¹⁷⁷Lu-complex of a compound of Formula (I) designed to characterize safety and biodistribution in patients diagnosed with gastric, colorectal, pancreatic cancer or other solid tumor malignancies. In some embodiments, a ⁶⁸Ga or ¹⁷⁷Lu-complex of a compound of Formula (I) is used to localize PAR2-expressing lesions and identify patients with PAR2-expressing gastric, colorectal, pancreatic cancers or other solid tumor malignancies who may benefit from treatment with PAR2-targeting therapeutic agents, such as ¹⁷⁷Lu-complex of a compound of Formula (I). In healthy humans, PAR2 has been implicated in proliferation, inflammation and regeneration and has been a drug target for treatment of inflammatory diseases. PAR2 is expressed in a broad variety of solid tumors including but not limited to gastric cancer, colorectal cancer, pancreatic cancer, renal cell carcinoma, bladder cancer, liver cancer, cervical cancer, endometrial cancer and ovarian cancer

This study will enroll approximately 28 evaluable subjects in total. Patients with metastatic gastric cancer, colorectal cancer, pancreatic cancer, or other solid tumor malignancies are eligible to participate if they meet all inclusion criteria and none of the exclusion criteria.

Study Populations: Patients with metastatic gastric cancer, colorectal cancer, pancreatic cancer, and other metastatic solid tumor malignancies.

Primary Objectives: To describe the safety of ⁶⁸Ga and ¹⁷⁷Lu-complex of a compound of Formula (I).

Secondary Objectives: To describe the biodistribution of a ⁶⁸Ga and ¹⁷⁷Lu-complex of a compound of Formula (I), to compare Formula (I) positron emission tomography/computed tomography (PET/CT) and single emission photon tomography/computed tomography (SPECT/CT) scans to standard of care anatomic imaging in detecting tumor lesions.

Primary Endpoints: Incidence of adverse events (AE) characterized overall and by type, frequency, seriousness, relationship to the study drug, timing, and severity, graded according to the National Cancer Institute (NCI) Common Terminology Criteria for Adverse Events (CTCAE), Version 5.0.

Secondary Endpoints: Maximum standard uptake value (SUVₘₐₓ) of each tumor and SUVₘₑₐₙ of organs. Ratio of the tumor SUV over reference region SUV. Number and location of tumors identified by a ⁶⁸Ga and ¹⁷⁷Lu-complex of a compound of Formula (I) on PET/CT or SPECT/CT, and concordance rate between ⁶⁸Ga and ¹⁷⁷Lu-complex of a compound of Formula (I) PET/CT and standard of care images.

Study Design: This study will enroll up to approximately 28 evaluable patients. Patients who receive study drug and complete all scheduled imaging procedures are considered evaluable.

There are four patient cohorts in the study: Cohort 1: patients with metastatic gastric cancer; Cohort 2: patients with metastatic colorectal cancer; Cohort 3: patients with metastatic pancreatic cancer; Cohort 4: patient with solid malignancies. Patients are enrolled into the four cohorts in parallel. Each cohort will enroll approximately 7 evaluable patients.

Each eligible patient enrolled will receive a single intravenous injection of a ⁶⁸Ga-complex of a compound of Formula (I) on Day 1 of the study at an activity of approximately 5 mCi (185 MBq). Some patients may receive low-dose ¹⁷⁷Lu-complex of a compound of Formula (I). Patients will be imaged with PET/CT following ⁶⁸Ga-complex administration and with SPECT/CT after ¹⁷⁷Lu-complex administration at the timepoints specified in the protocol.

All patients must meet all the inclusion eligibility criteria and none of the exclusion eligibility criteria, as appropriate and provided below.

Inclusion Criteria: Metastatic gastric cancer (Cohort 1 patients); metastatic colorectal cancer (Cohort 2 patients); metastatic pancreatic cancer (Cohort 3 patients); or solid tumor malignancies (Cohort 4 patients). Male or non-pregnant, non-lactating female subjects age≥18 years. Subjects who are sexually active must agree to use adequate method(s) of effective contraception during their participation in the study. Eastern Cooperative Oncology Group (ECOG) Performance Status≤2. Adequate hepatic function as defined by a) serum alanine aminotransaminase (ALT)/aspartate aminotransaminase (AST)≤3×upper limit of normal (ULN) or ≤5×ULN if liver metastases are present or received prior mitotane therapy, and b) serum bilirubin—total≤1.5×ULN (unless due to Gilbert's syndrome or hemolysis in which case total≤3.0×ULN). Adequate renal function as measured by creatinine clearance calculated by the Cockcroft-Gault formula (≥45 mL/minute). Able to understand and willing to sign written informed consent.

Exclusion Criteria: Administered a radionuclide within a period of time corresponding to less than 10 physical half-lives of the radionuclide prior to study Day 1. Radiotherapy≤14 days prior to study Day 1. Major surgery≤21 days prior to study Day 1 or has not recovered from adverse effects of such procedure. History of cerebrovascular accident within 6 months or that resulted in ongoing neurologic instability. History of other previous or concurrent cancer that would interfere with the determination of safety. Any other condition that in the opinion of the Investigator would place the subject at an unacceptable risk or cause the subject to be unlikely to fully participate or comply with study procedures.

### Study Drug, Dose, and Mode of Administration

Study Drug: In some embodiments, the study drug is a ⁶⁸Ga-complex of a compound of Formula (I) (e.g., a ⁶⁸Ga-complex of any one of compounds 219 to 543). In some embodiments, the study drug is a ¹⁷⁷Lu-complex of a compound of Formula (I) (e.g., a ¹⁷⁷Lu-complex of any one of compounds 219 to 543).

Dose: 5.0 mCi (±20%) for the ⁶⁸Ga-complex, 27 mCi for the ¹⁷⁷Lu-complex. Total carrier mass of the compound of Formula (I): not more than (NMT) 90 μg/dose.

Mode of administration: Intravenous

Duration of Participation: A 28-day screening window will be utilized where the subject will undergo study assessments to deem the subject eligible for the study. Once confirmed, subjects will receive the study drug, a ⁶⁸Ga or ¹⁷⁷Lu-complex of a compound of Formula (I), and PET/CT or SPECT/CT imaging on days described in protocol. The subjects will have a safety follow-up up to 7 days after last imaging assessment.

Study Duration: The start of the study will be the date on which the first subject provides informed consent. The end of the study will be when the database is locked.

The examples and embodiments described herein are for illustrative purposes only and various modifications or changes suggested to persons skilled in the art are to be included within the purview of this application and scope of the appended claims. Other embodiments are set forth in the following claims, along with the full scope of equivalents to which such claims are entitled.

What is claimed is:

1. A compound of Formula (VIII): 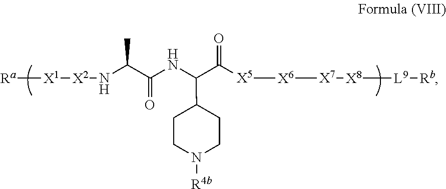
or a pharmaceutically acceptable salt thereof, wherein:
L⁹ is an optional linker that is attached to any one of X⁵, X⁶, X⁷, or X⁸;
R^{b} is:
1,4,7,10-tetraazacyclododecane-1,4,7,10-tetraacetic acid (DOTA);
2,2′,2″-(10-(2-amino-2-oxoethyl)-1,4,7,10-tetraazacyclododecane-1,4,7-triyl)triacetic acid (PSC);
1,4,7,10-tetraazacyclododecane-1,4,7-triacetic acid (DO3A);
1,4,7,10-tetraazacyclododecane-1,7-diacetic acid (DO2A);
α,α′,α″,α′″-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid;
benzyl-1,4,7,10-tetraazacyclododecane-1,4,7,10-tetraacetic acid (Bn-DOTA);
2,2′,2″-(1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2,2′,2″,2′-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H₄octapa-benzyl; or
3,6,9,12-tetrakis(carboxymethyl)-3,6,9,12-tetraazatetradecanedioic acid (TTHA);
R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, or —R¹¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, or —NO₂;
X¹ is 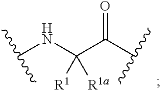
R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl], 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form: 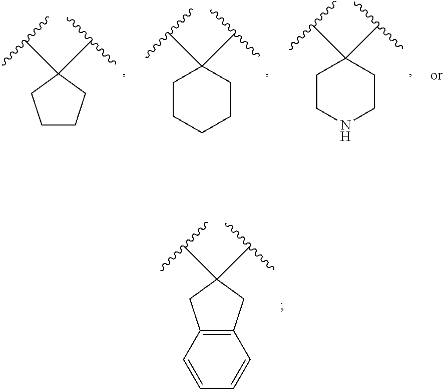 [SMILES: *C1(*)CCCC1 ; *C1(*)CCCCC1 ; *C1(*)CCNCC1 ; *C1(*)Cc2ccccc2C1]
X² is 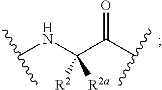
R² is -L²-R^{2b};
L² is absent;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl], 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form: 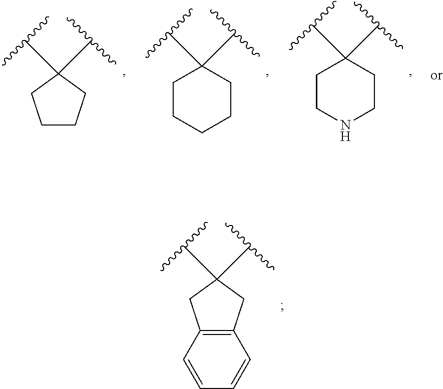 [SMILES: *C1(*)CCCC1 ; *C1(*)CCCCC1 ; *C1(*)CCNCC1 ; *C1(*)Cc2ccccc2C1]
R^{4b} is H, —C(═NH)NH₂, —C(═O)CH(CH₃)NCH₃, —CH₂C(═O)NHC₁-C₁₀ alkyl-NH₂, or —C(═O)CH₂NHC₁-C₁₀ alkyl;
X⁵ is absent, -L⁵-, alanine (Ala), lysine (Lys), ornithine (Orn), β³-homolysine, γ-glutamic acid (γ-Glu), leucine (Leu), phenylalanine (Phe), 4-azidophenylalanine (Phe(4-N₃)), norleucine (Nle), 2-aminoheptanoic acid, 2-amino-6-azidohexanoic acid, N6-acetyl-lysine, N6-(pent-4-ynoyl)-lysine, N6-octanoyl-lysine, tyrosine (Tyr), N6-palmitoyl-Lys, N6-(15-carboxypentadecanoyl)-Lys, N6-(16-oxo-16-((2-(N—N—N-trimethyl-amino)ethyl)amino)hexadecanoyl)-Lys, N6-(4-fluorobenzoyl)-Lys, β³-homolysine, 3-amino-alanine (H-Dap-OH), 4-fluorophenylalanine (Phe(4-F)), 4-iodophenylalanine (Phe(4-I)), 4-nitrophenylalanine (Phe(4-NO₂)), (4-fluorobenzamido)methylglycine, 4-aminomethylphenylalanine (Phe(4-CH₂NH₂)), 4-azidomethylphenylalanine (Phe(4-CH₂N₃)), 4-amino-1-((3-fluoro-4-methylbenzyl)glycyl)piperidine-4-carboxylic acid, 7-hydroxy-1,2,3,4-tetrahydroisoquinoline-3-carboxylic acid, proline (Pro), hydroxyproline (Hyp), piperidine-2-carboxylic acid, isoindoline-1-carboxylic acid, or (4-(aminomethyl)-1H-1,2λ²,3λ²-triazol-1-yl)methyl)-glycine;
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or 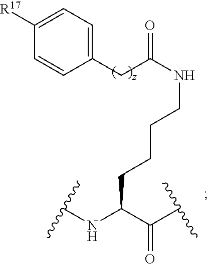
R¹⁷ is —CH₃ or —I;
z is 1, 2, or 3; and
X⁸ is absent, lysine (Lys), or glutamic acid (Glu).
L⁵, L⁶, and L⁷ are each independently —NR¹⁵-L¹³-L¹⁴-C(═O)— or substituted or unsubstituted piperazinylene-;
L¹³ is absent, —C₁-C₆ alkylene, substituted or unsubstituted heterocycloalkylene, substituted or unsubstituted cyclohexylene, —(CH₂CH₂O)_{y}—, or —(CH₂CH₂NR¹⁶)_{y}—;
L¹⁴ is absent, —C₁-C₆ alkylene, substituted or unsubstituted heterocycloalkylene, substituted or unsubstituted cyclohexylene;
R¹⁵ and R¹⁶ are each independently selected from H or —C₁-C₄ alkylene; and
y is 1, 2, 3, 4, 5, or 6;
wherein the compound of Formula (VIII) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (VIII) may be either —COOH or —CONH₂;
wherein the radionuclide of the radionuclide complex is:
111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 99m-technetium (^{99m}Tc), 195m-platinum (^{195m}Pt), 225-actinium (²²⁵Ac), 213-bismuth (²¹³Bi), 223-radium (²²³Ra), 211-astatine (²¹¹At), aluminium-[¹⁸F]fluoride ([¹⁸F]AlF), 90-yttrium (⁹⁰Y), 175-lutetium (¹⁷⁵Lu), 177-lutetium (¹⁷⁷Lu), 161-terbium (¹⁶¹Tb), 186-rhenium (¹⁸⁶Re), 188-rhenium (¹⁸⁸Re), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), 153-samarium (¹⁵³Sm), 89-strontium (⁸⁹Sr), 198-gold (¹⁹⁸Au), 169-erbium (¹⁶⁹Er), 165-dysprosium (¹⁶⁵Dy), 99m-technetium (^{99m}Tc), 89-zirconium (⁸⁹Zr), 52-manganese (⁵²Mn), 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

2. The compound of claim 1, wherein the compound of Formula (VIII) has the structure of Formula (VIII-1): 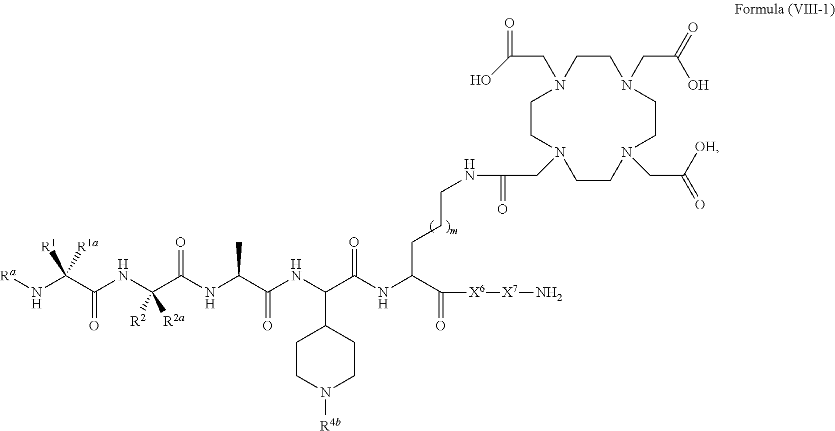
or a pharmaceutically acceptable salt thereof, wherein:
m is 1 or 2.

3. The compound of claim 1, wherein:
R^{a} is —C(═O)—R¹⁰, wherein R¹⁰ is a monocyclic heteroaryl selected from the group consisting of pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl; and wherein R¹⁰ is optionally substituted with 1 to 3 substituents that are each independently selected from the group consisting of halo, C₁-C₆ alkyl, alkoxy, —OH, —NH₂, —NH—C₁-C₆ alkylene-R¹¹, and —O—C₁-C₆ alkylene-R¹¹.

4. The compound of claim 1, wherein:
R^{a} is 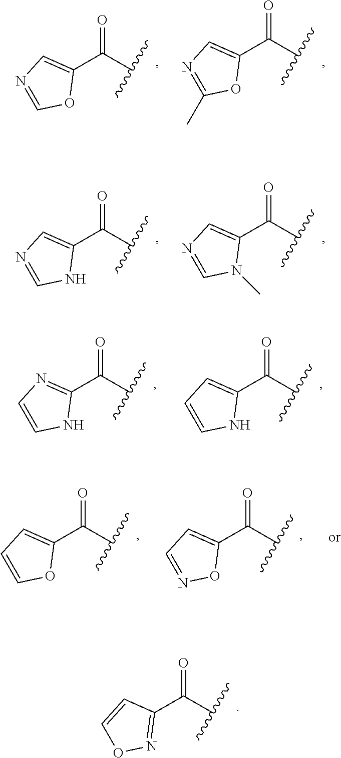
and
R^{4b} is H or —C(═NH)NH₂.

5. The compound of claim 1, wherein:
R¹ is -L¹-R^{1b};
L¹ is absent or —CH₂—;
R^{1b} is cyclopentyl, cyclohexyl, cycloheptyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, 2-chlorophenyl, 3-chlorophenyl, 4-chlorophenyl, 2,4-dichlorophenyl, 3,4-dichlorophenyl, or 2,5-dichlorophenyl; and
R^{1a} is hydrogen or methyl;
R² is -L²-R^{2b};
L² is absent or —CH₂—;
R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl], or 4′-methyl-[1,1′-biphenyl]-4-yl; and
R^{2a} is hydrogen or methyl;
R² and R^{2a} are taken together with the carbon atoms to which they are attached to form: 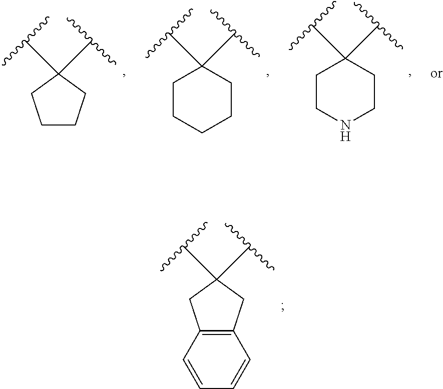 [SMILES: *C1(*)CCCC1 ; *C1(*)CCCCC1 ; *C1(*)CCNCC1 ; *C1(*)Cc2ccccc2C1]
and
R^{4b} is H or —C(═NH)NH₂.

6. The compound of claim 1, wherein: 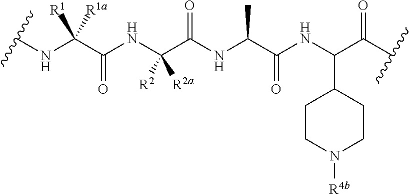
is:

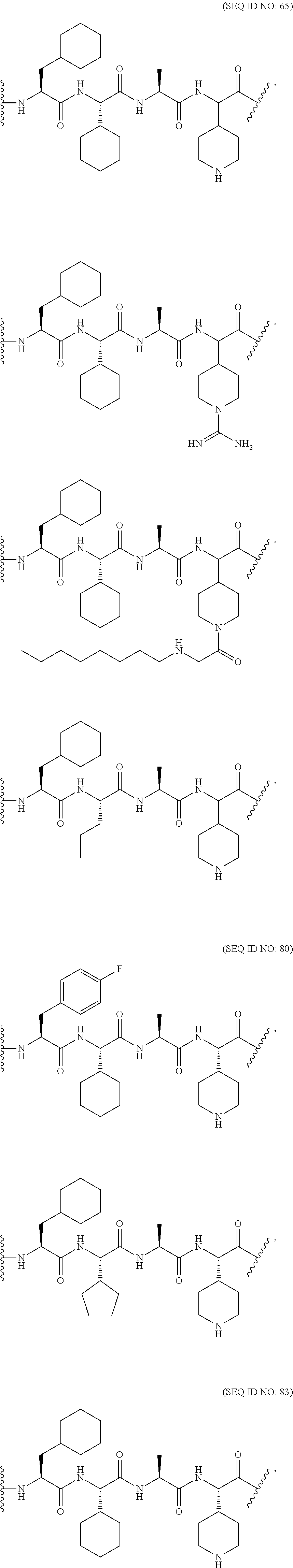

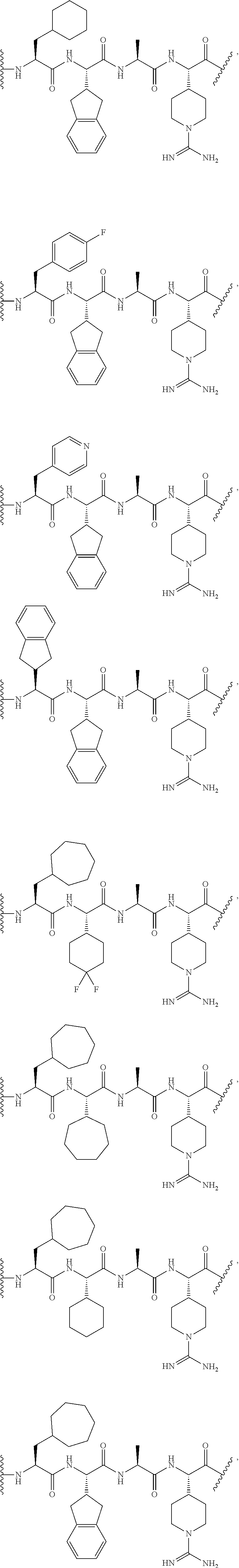

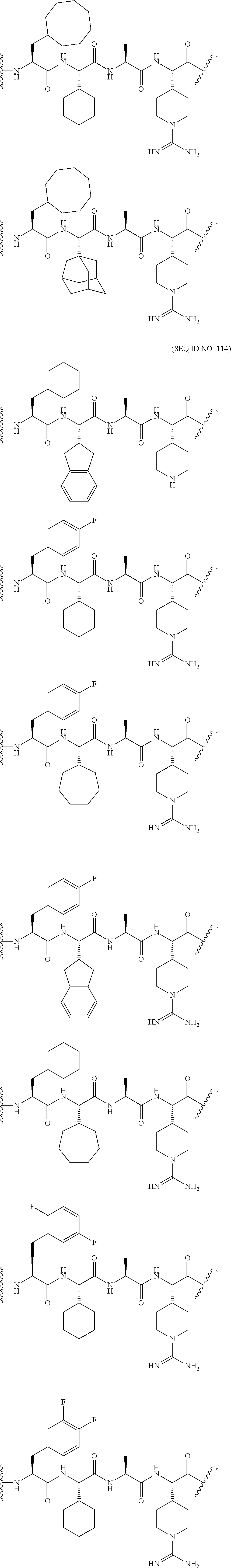

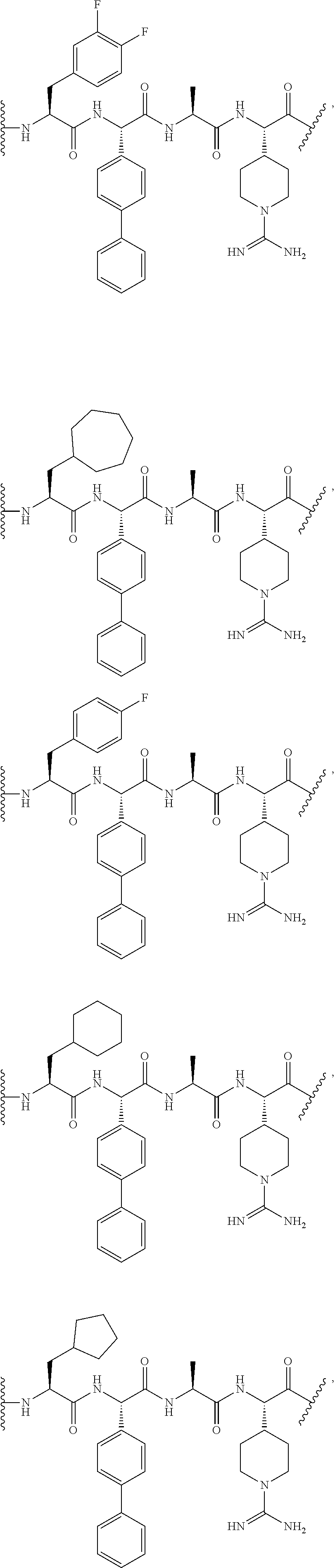

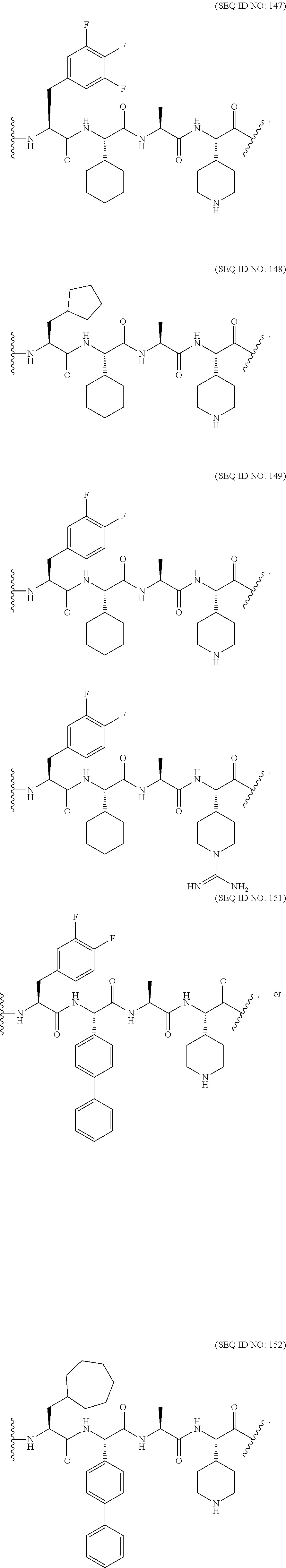

7. The compound of claim 1, wherein:
X⁵ is -L⁵-, (S)-alanine (Ala), (S)-lysine (Lys), (S)-ornithine (Orn), or (S)-(4-fluorobenzamido)methylglycine.

8. The compound of claim 1, wherein:
X⁶ is absent, (S)-lysine (Lys), (S)-ornithine (Orn), (R)-lysine (Lys), (R)-ornithine (Orn),

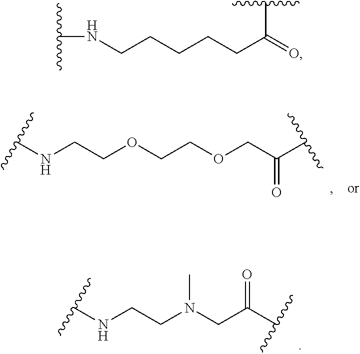

9. The compound of claim 1, wherein:
X⁶ is absent, -L⁶-, lysine (Lys), or ornithine (Orn);
X⁷ is absent, lysine (Lys), ornithine (Orn), or 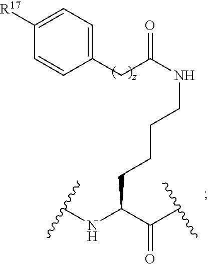
and
X⁸ is absent.

10. The compound of claim 1, wherein:
L⁹ is absent.

11. The compound of claim 1, wherein:
L⁹ is —C(═O)—C₁-C₆ alkylene-NR¹⁴—*, —C(═O)—NR¹³—C₁-C₆ alkylene-NR¹⁴—*, —C(═O)—C₁-C₆ alkylene-(substituted or unsubstituted heterocycloalkylene)-NR¹⁴—*, —C(═O)-(substituted or unsubstituted heterocycloalkylene)-C₁-C₆alkylene-NR¹⁴—*, —C(═O)—C₁-C₆ alkylene-(substituted or unsubstituted cyclohexylene)-NR¹⁴—*, —C(═O)-(substituted or unsubstituted cyclohexylene)-C₁-C₆ alkylene-NR¹⁴—*, —C(═O)—C₁-C₆ alkylene-(OCH₂CH₂)ₓ—NR¹⁴—*; —CH₂—C(═O)—C₁-C₆ alkylene-NR¹⁴—*, —CH₂—C(═O)—NR¹³—C₁-C₆ alkylene-NR¹⁴—*, —CH₂—C(═O)—C₁-C₆ alkylene-(substituted or unsubstituted heterocycloalkylene)-NR¹⁴—*, —CH₂—C(═O)-(substituted or unsubstituted heterocycloalkylene)-C₁-C₆ alkylene-NR¹⁴—*, —CH₂—C(═O)—C₁-C₆ alkylene-(substituted or unsubstituted cyclohexylene)-NR¹⁴—*, —CH₂—C(═O)-(substituted or unsubstituted cyclohexylene)-C₁-C₆ alkylene-NR¹⁴—*, or —CH₂—C(═O)—C₁-C₆ alkylene-(OCH₂CH₂)ₓ—NR¹⁴—*;
R¹³ and R¹⁴ are each independently selected from H or —C₁-C₄ alkyl; and
x is 1, 2, 3, 4, 5, or 6;
wherein * denotes the attachment point to R^{b}.

12. The compound of claim 1, wherein:
L⁹ is a bond,

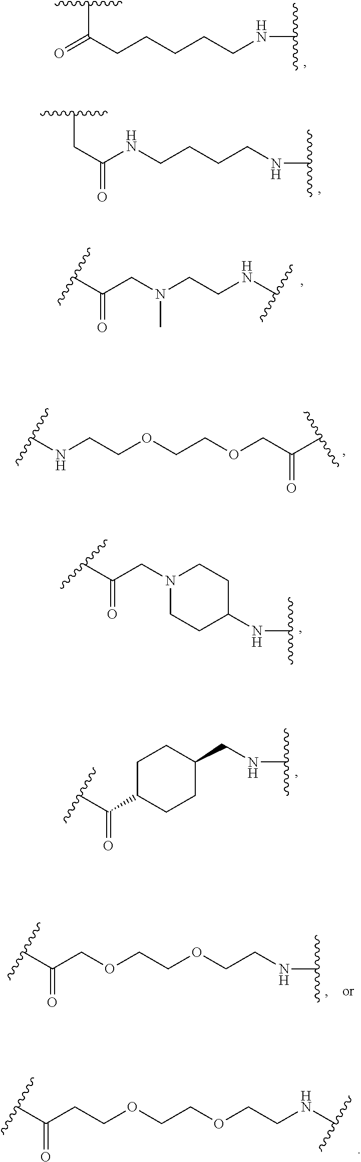

13. The compound of claim 1, wherein:
R^{b} is 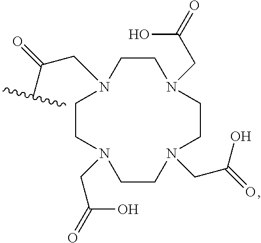 [SMILES: *C(=O)CN1CCN(CC(=O)O)CCN(CC(=O)O)CCN(CC(=O)O)CC1]
or a radionuclide complex thereof.

14. A pharmaceutical composition comprising a compound of claim 1, or a pharmaceutically acceptable salt thereof, and at least one pharmaceutically acceptable excipient.

15. A method for the treatment of cancer comprising administering to a mammal with cancer an effective amount of a compound of claim 1, or a pharmaceutically acceptable salt thereof; wherein the cancer comprises tumors and the tumors overexpress Protease-Activated Receptor-2 (PAR2); and wherein the compound is a radionuclide complex and the radionuclide of the radionuclide complex is 225-actinium (²²⁵Ac), 177-lutetium (¹⁷⁷Lu), 161-terbium (¹⁶¹Tb), 212-lead (²¹²Pb), 67-copper (⁶⁷Cu), 90-yttrium (⁹⁰Y), or 213-bismuth (²¹³Bi), wherein the cancer is colon cancer, pancreatic cancer, stomach cancer, esophageal cancer, rectal cancer, liver cancer, or lung cancer.

16. A method for the in vivo imaging of tissues or organs in a mammal with tumors expressing Protease-Activated Receptor-2 (PAR2) comprising administering to the mammal a compound of claim 1, or a pharmaceutically acceptable salt thereof; and performing positron emission tomography (PET) analysis or single-photon emission computerized tomography (SPECT); wherein the compound of claim 1, or a pharmaceutically acceptable salt thereof, comprises a diagnostic radionuclide that is 177-lutetium (¹⁷⁷Lu), 111-indium (¹¹¹In), 64-copper (⁶⁴Cu), 67-gallium (⁶⁷Ga), 68-gallium (⁶⁸Ga), 99m-technetium (^{99m}Tc), or 195m-platinum (^{195m}Pt).

17. A compound of Formula (IX): 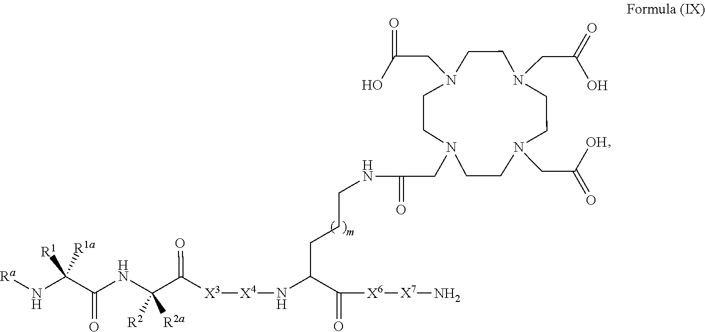
or a pharmaceutically acceptable salt thereof, wherein:
m is 1 or 2;
R^{a} is —C(═O)—R¹⁰;
R¹⁰ is an aryl, heteroaryl, or partially unsaturated bicyclic heterocyclic ring, each optionally substituted with 1 to 3 substituents that are each independently selected from the group consisting of halo, —C₁-C₆ alkyl, alkoxy, —OH, —NH₂, —NH—C₁-C₆ alkylene-R¹¹, or —O—C₁-C₆ alkylene-R¹¹, or —R¹;
wherein any saturated carbon of R¹⁰ is optionally substituted with oxo (═O);
R¹¹ is an aryl or heteroaryl optionally substituted with 1 to 3 substituents that are each independently selected from the group consisting of —F, —Cl, —Br, —C₁-C₃ alkyl, —CF₃, —CN, —OCH₃, —NH₂, or —NO₂;
R¹ is -L¹-R^{1b};
L¹ is absent, —CH₂—, or —CHPh-;
R^{1b} is cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, pyridin-2-yl, pyridin-3-yl, pyridin-4-yl, 4-aminophenyl, 4-carbamoylphenyl, 4-ureidophenyl, 4-acetylphenyl, 4-azidophenyl, 4-(azidomethyl)phenyl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl], 4′-methyl-[1,1′-biphenyl]-4-yl, piperidin-1-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{1b} is optionally additionally substituted with 1 or more —F, —Br, —Cl, —I, —CF₃, or —C₁-C₆ alkyl;
R^{1a} is hydrogen or methyl;
or R¹ and R^{1a} are taken together with the carbon atoms to which they are attached to form: 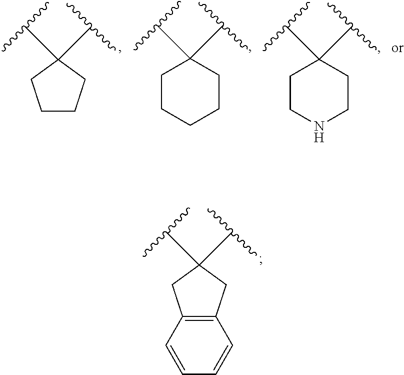 [SMILES: *C1(*)CCCC1 ; *C1(*)CCCCC1 ; *C1(*)CCNCC1 ; *C1(*)Cc2ccccc2C1]
R² is -L²-R^{2b};
L² is absent, —CH₂—, or —CHPh-;
R^{2b} is methyl, pent-3-yl, tert-butyl, 1,1,1,3,3,3-hexafluoropropan-2-yl, cyclopropyl, cyclobutyl, cyclopentyl, cyclohexyl, 3-fluorocyclohexyl, 4-fluorocyclohexyl, 3,3-difluorocyclohexyl, 3,3-dimethylcyclohexyl, 4,4-difluorocyclohexyl, 4,4-dimethylcyclohexyl, cycloheptyl, cyclooctyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, 3,4,5-trichlorophenyl, 4-bromophenyl, 4-methylphenyl, 4-trifluoromethylphenyl, 3,4-bis(trifluoromethyl)phenyl, 4-vinylphenyl, 4-tert-butylphenyl, 2-hydroxyphenyl, 4-hydroxyphenyl, 4-methoxyphenyl, 4-aminophenyl, 4-carbamoylphenyl, 4-ureidophenyl, 4-acetylphenyl, 4-azidophenyl, 4-(azidomethyl)phenyl, pyridin-2-yl, pyridin-3-yl, pyridin-4-yl, 2,3-dihydro-1H-inden-1-yl, 2,3-dihydro-1H-inden-2-yl, 3-cyclopropylphenyl, [1,1′-biphenyl]-4-yl, 4′-fluoro-[1,1′-biphenyl], 4′-methyl-[1,1′-biphenyl]-4-yl, 1,2,3,4-tetrahydronaphthalen-1-yl, 5,6,7,8-tetrahydronaphthalen-2-yl, 1H-indol-3-yl, 2,3-dihydrobenzofuran-6-yl, naphthalen-1-yl, or adamantan-1-yl;
wherein each R^{2b} is optionally additionally substituted with 1 or more —F, —Br, —Cl, —I or —C₁-C₆ alkyl;
R^{2a} is hydrogen or methyl;
or R² and R^{2a} are taken together with the carbon atoms to which they are attached to form: 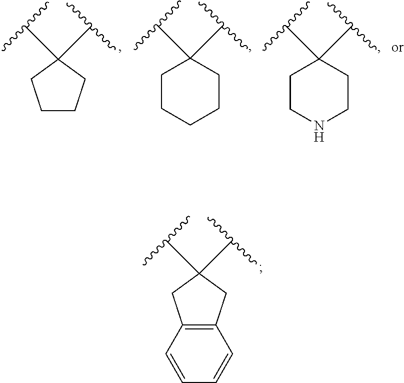 [SMILES: *C1(*)CCCC1 ; *C1(*)CCCCC1 ; *C1(*)CCNCC1 ; *C1(*)Cc2ccccc2C1]
X³ is glycine, (S)-alanine (Ala), (S)-trifluoromethylalanine, (S)-homoalanine, (S)-Glu, (S)-Gln, (S)-Orn, (S)-lysine (Lys), (S)-serine (Ser), (S)—N-methylalanine (N-Me-Ala), 2-amino-2-methylpropanoic acid (AIB), 1-aminocyclopropane-1-carboxylic acid (ACC), norleucine (Nle), (S)-3-aminoalanine, (S)-2-amino-6-azidohexanoic acid, (S)-2-amino-6-(4-hexyl-1H-1,2,3-triazol-1-yl)hexanoic acid, (S)-2-amino-6-(4-((2-methoxyethoxy)methyl)-1H-1,2,3-triazol-1-yl)hexanoic acid, (S)-2-amino-6-(4-(cyclopentylmethyl)-1H-1,2,3-triazol-1-yl)hexanoic acid, (S)-2-amino-6-(4-benzyl-1H-1,2,3-triazol-1-yl)hexanoic acid, (S)-proline (Pro), (S)-cyclopropylglycine, or (S)-biphenylalanine (Bip);
X⁴ is 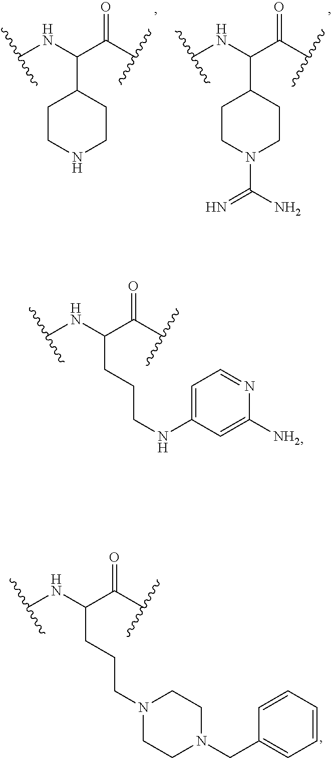 [SMILES: *NC(C(*)=O)C1CCNCC1 ; *NC(C(*)=O)C1CCN(C(=N)N)CC1 ; *NC(CCCNc1ccnc(N)c1)C(*)=O ; *NC(CCCN1CCN(Cc2ccccc2)CC1)C(*)=O] 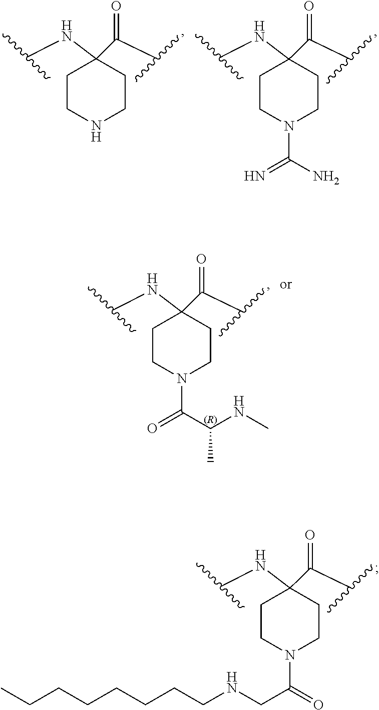
X⁶ is absent, -L⁶-, lysine (Lys), ornithine (Orn), (4-fluorobenzamido)methylglycine, N6-(4-fluorobenzoyl)-Lys, 4-amino-1-(methylglycyl)piperidine-4-carboxylic acid, or 4-amino-1-(N-carbamimidoyl-N-methylglycyl)piperidine-4-carboxylic acid;
X⁷ is absent, -L⁷-, lysine (Lys), ornithine (Orn), glutamic acid (Glu), norleucine (Nle), or 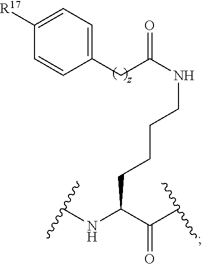
R¹⁷ is —CH₃ or —I;
z is 1, 2, or 3; and
L⁶ and L⁷ are each independently —NR¹⁵-L¹³-L¹⁴-C(═O)— or substituted or unsubstituted piperazinylene-;
L¹³ is absent, —C₁-C₆ alkylene, substituted or unsubstituted heterocycloalkylene, substituted or unsubstituted cyclohexylene, —(CH₂CH₂O)_{y}—, or —(CH₂CH₂NR¹⁶)_{y}—;
L¹⁴ is absent, —C₁-C₆ alkylene, substituted or unsubstituted heterocycloalkylene, substituted or unsubstituted cyclohexylene;
R¹⁵ and R¹⁶ are each independently selected from H or —C₁-C₄ alkylene; and
y is 1, 2, 3, 4, 5, or 6;
wherein the compound of Formula (IX) is optionally substituted with —C(═O)—C₁-C₁₄ alkylene-R¹², wherein R¹² is H, —CH₃, —COOH, —OH, —NH₂, or —NHCH₂CH₂N(CH₃)₃;
wherein any free —NH— of a peptide bond is optionally independently substituted with —CH₃ or —CH₂CH₃;
wherein any alpha position of any amino acid is optionally independently substituted with —CH₃ or —CH₂CH₃; and
wherein the C-terminal end of the compound of Formula (IX) may be either —COOH or —CONH₂;
wherein the radionuclide of the radionuclide complex is:
an Auger electron-emitting radionuclide that is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 99m-technetium (^{99m}Tc), 195m-platinum (^{195m}Pt) 225-actinium (²²⁵Ac), 213-bismuth (²¹³Bi), 223-radium (²²³Ra), 211-astatine (²¹¹At), aluminium-[¹⁸F]fluoride ([¹⁸F]AlF), 90-yttrium (⁹⁰Y), 175-lutetium (¹⁷⁵Lu), 177-lutetium (¹⁷⁷Lu), 161-terbium (¹⁶¹Tb), 186-rhenium (¹⁸⁶Re), 188-rhenium (¹⁸⁸Re), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), 67-copper (⁶⁷Cu), 153-samarium (¹⁵³Sm), 89-strontium (⁸⁹Sr), 198-gold (¹⁹⁸Au), 169-erbium (¹⁶⁹Er), 165-dysprosium (¹⁶⁵Dy), 99m-technetium (^{99m}Tc), 89-zirconium (⁸⁹Zr), 52-manganese (⁵²Mn), 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

18. The compound of claim 17, wherein:
R^{a} is

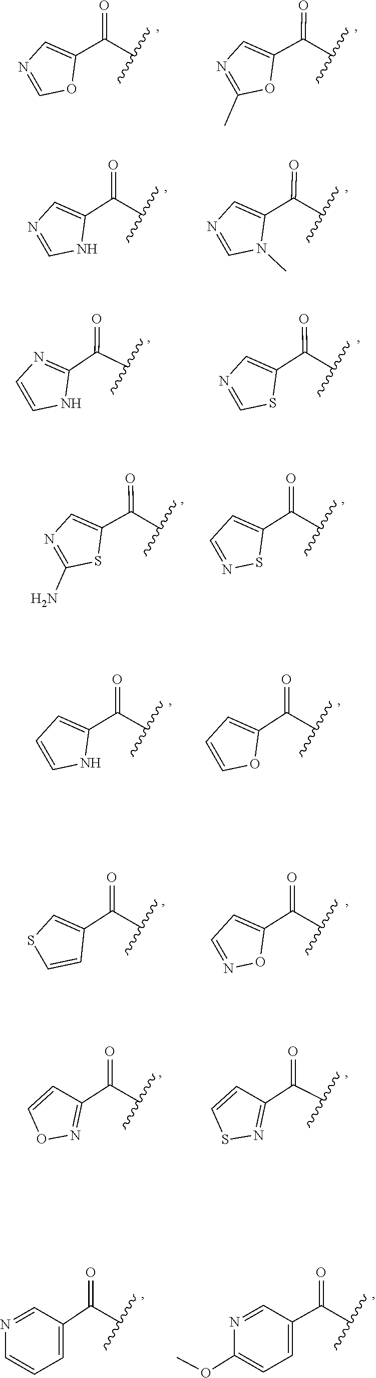

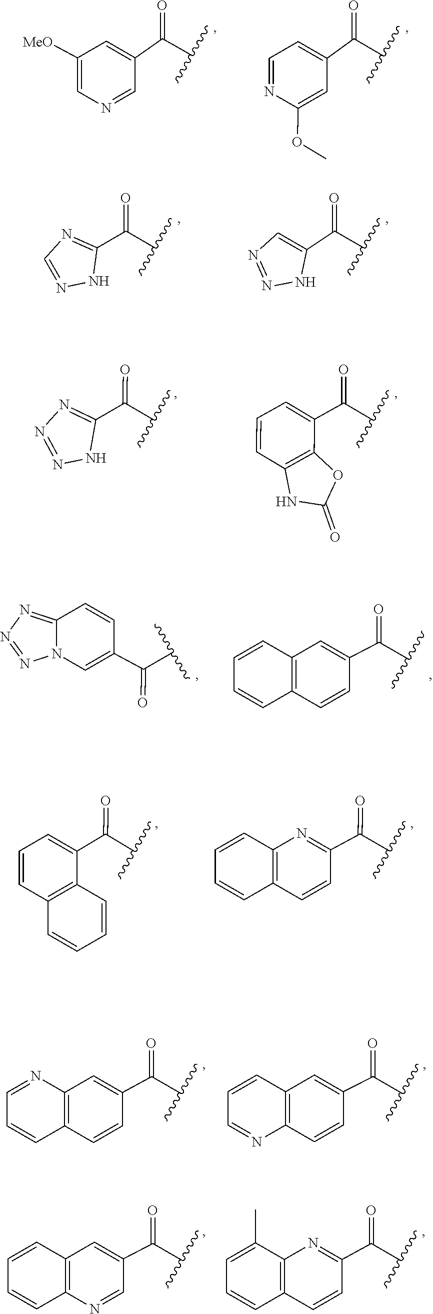

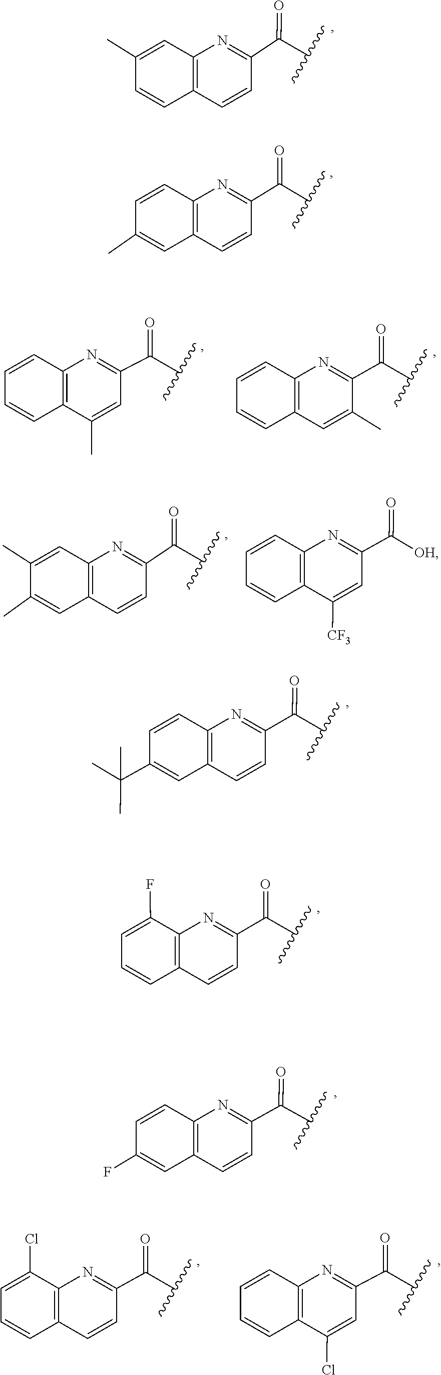

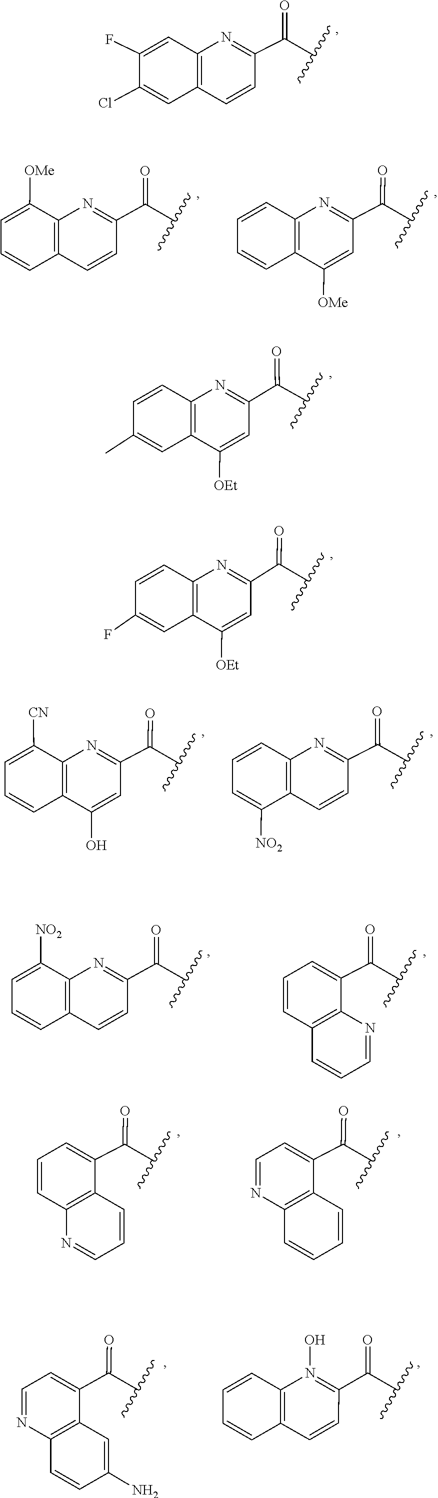

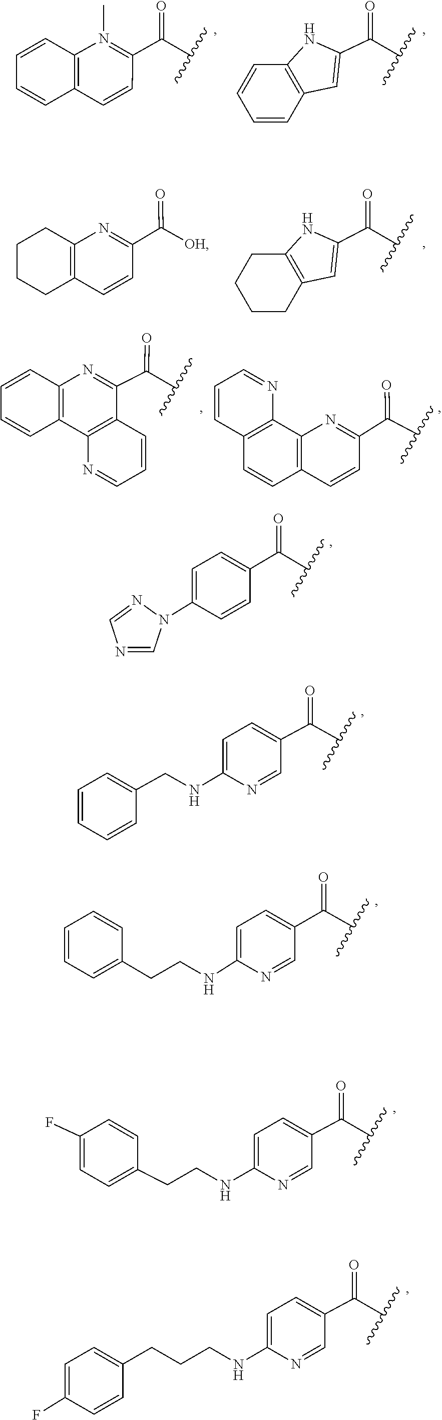

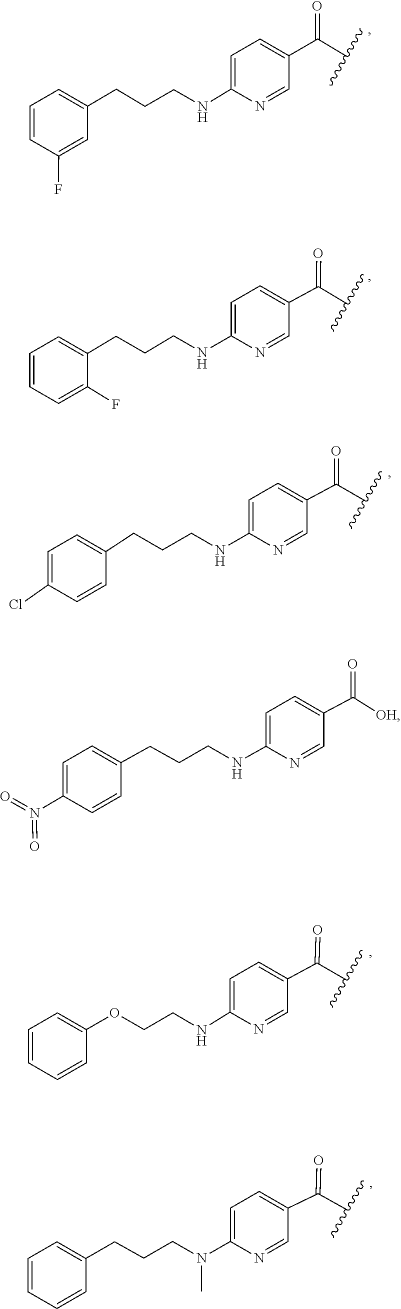 [SMILES: *C(=O)c1ccc(NCCCc2cccc(F)c2)nc1 ; *C(=O)c1ccc(NCCCc2ccccc2F)nc1 ; *C(=O)c1ccc(NCCCc2ccc(Cl)cc2)nc1 ; O=C(O)c1ccc(NCCCc2ccc([N+](=O)[O-])cc2)nc1 ; *C(=O)c1ccc(NCCOc2ccccc2)nc1 ; *C(=O)c1ccc(N(C)CCCc2ccccc2)nc1]

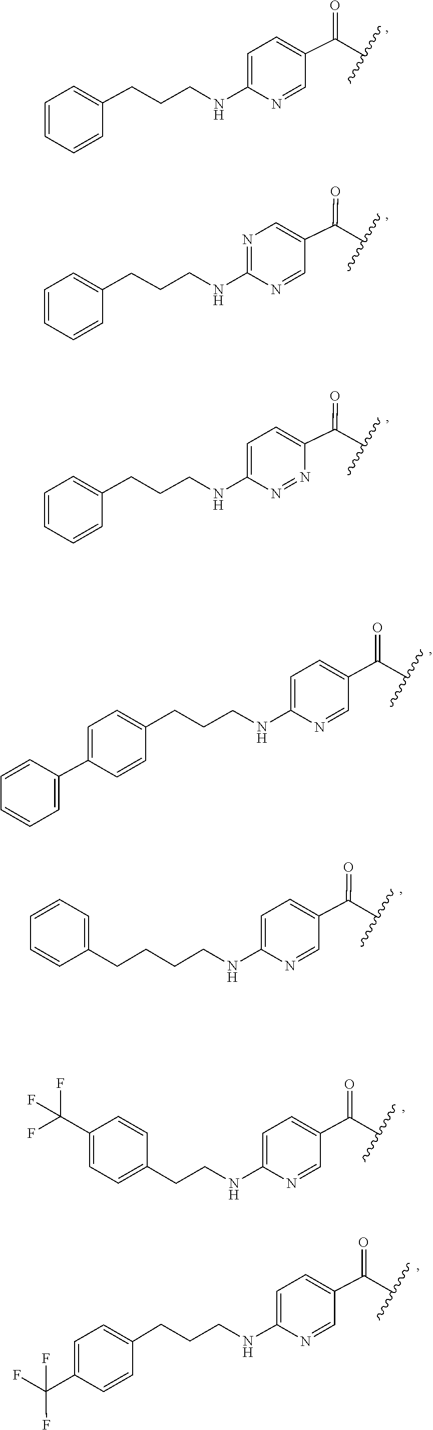

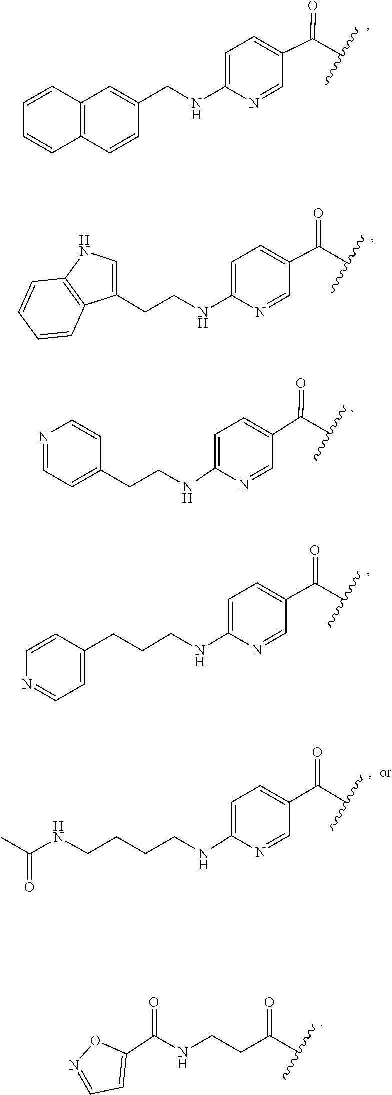 [SMILES: *C(=O)c1ccc(NCc2ccc3ccccc3c2)nc1 ; *C(=O)c1ccc(NCCc2c[nH]c3ccccc23)nc1 ; *C(=O)c1ccc(NCCc2ccncc2)nc1 ; *C(=O)c1ccc(NCCCc2ccncc2)nc1 ; *C(=O)c1ccc(NCCCCNC(C)=O)nc1 ; *C(=O)CCNC(=O)c1ccno1]

19. The compound of claim 17, wherein:
R^{a} is

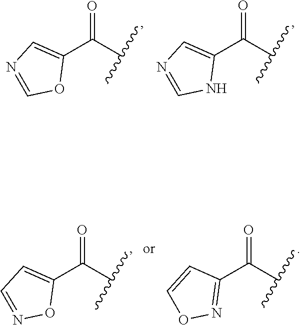

20. The compound of claim 17, wherein the compound has the following structure: 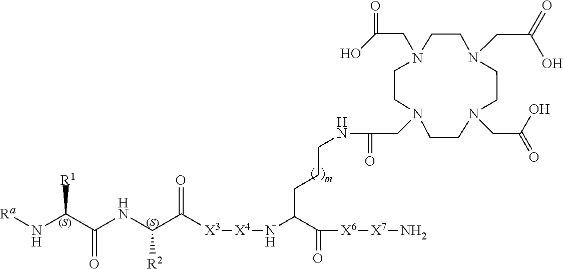
R¹ is -L¹-R^{1b};
L¹ is —CH₂—; and
R^{1b} is cyclopentyl, cyclohexyl, cycloheptyl, cyclooctyl, phenyl, 4-fluorophenyl, 2,4-difluorophenyl, 3,4-difluorophenyl, 2,5-difluorophenyl, 3,4,5-trifluorophenyl, perfluorophenyl, 2-chlorophenyl, 3-chlorophenyl, 4-chlorophenyl, 2,4-dichlorophenyl, 3,4-dichlorophenyl, 2,5-dichlorophenyl, pyridin-4-yl, 2,3-dihydro-1H-inden-2-yl, 3-cyclopropylphenyl, or [1,1′-biphenyl]-4-yl;
R² is R^{2b}; and R^{2b} is cyclohexyl or cycloheptyl; or
R² is R^{2b}; and R^{2b} is 2,3-dihydro-1H-inden-2-yl or [1,1′-biphenyl]-4-yl.

21. The compound of claim 17, wherein:
R¹ is -L¹-R^{1b};
L¹ is —CH₂—;
R^{1b} is cyclohexyl, cycloheptyl, 4-fluorophenyl, or 3,4-difluorophenyl;
R² is R^{2b};
R^{2b} is cyclohexyl, cycloheptyl, 2,3-dihydro-1H-inden-2-yl, or [1,1′-biphenyl]-4-yl.

22. The compound of claim 17, wherein:
X³ is (S)-alanine (Ala) or (S)-norleucine (Nle).

23. The compound of claim 17, wherein:
R¹ is -L¹-R^{1b};
L¹ is absent or —CH₂—;
R^{1b} is cyclopentyl, cyclohexyl, cycloheptyl, phenyl, 2-fluorophenyl, 3-fluorophenyl, 4-fluorophenyl, 2,4-difluorophenyl, 3,4-difluorophenyl, 2,5-difluorophenyl, 3,4,5-trifluorophenyl, 2-chlorophenyl, 3-chlorophenyl, 4-chlorophenyl, 2,4-dichlorophenyl, 3,4-dichlorophenyl, or 2,5-dichlorophenyl;
R^{1a} is hydrogen or methyl;
R² is R^{2b};
L² is absent;
R^{2b} is cyclopentyl, cyclohexyl, cycloheptyl, phenyl, 4-fluorophenyl, 2,3-dihydro-1H-inden-2-yl, [1,1′-biphenyl]-4-yl, 4′-fluoro-[1,1′-biphenyl], or 4′-methyl-[1,1′-biphenyl]-4-yl;
R^{2a} is hydrogen or methyl;
X³ is glycine, (S)-alanine (Ala), or norleucine (Nle);
X⁶ is absent, lysine (Lys), ornithine (Orn), 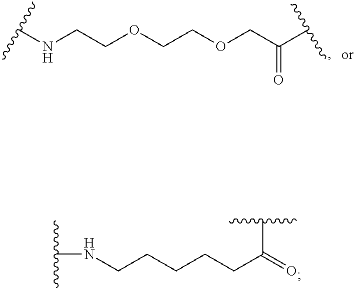 [SMILES: *NCCOCCOCC(*)=O ; *NCCCCCC(*)=O]
X⁷ is absent, lysine (Lys), ornithine (Orn), or 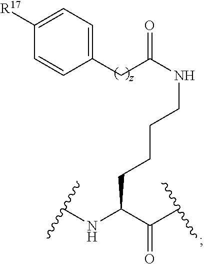
R¹⁷ is —CH₃ or —I;
z is 1, 2, or 3; and
X⁸ is absent.

24. The compound of claim 17, wherein:
X⁴ is 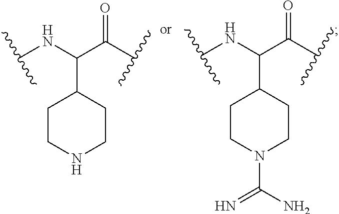 [SMILES: *NC(C(*)=O)C1CCNCC1 ; *NC(C(*)=O)C1CCN(C(=N)N)CC1]
or
X⁴ is 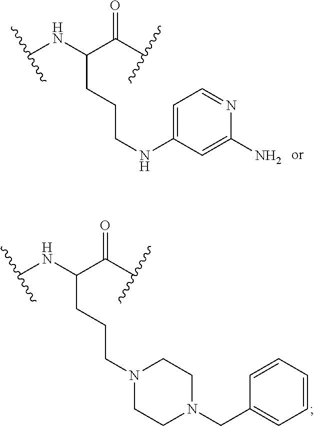 [SMILES: *NC(CCCNc1ccnc(N)c1)C(*)=O ; *NC(CCCN1CCN(Cc2ccccc2)CC1)C(*)=O]
or
X⁴ is

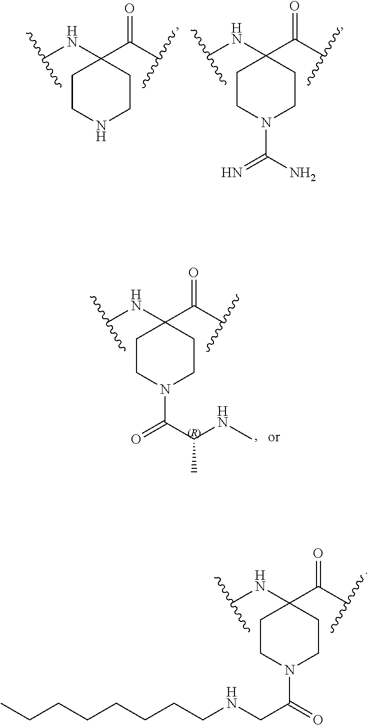

25. The compound of claim 17, wherein the compound of Formula (IX) has one of the following structures:

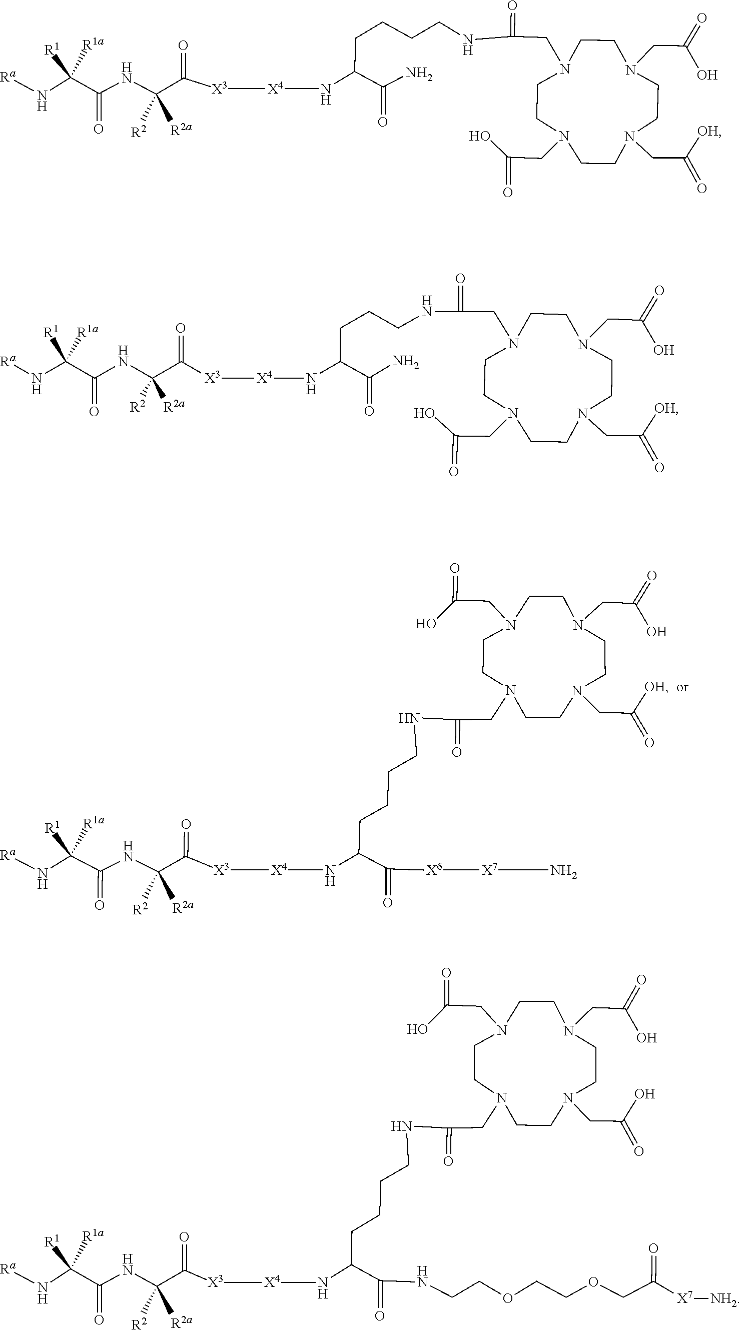

26. The compound of claim 17, wherein:

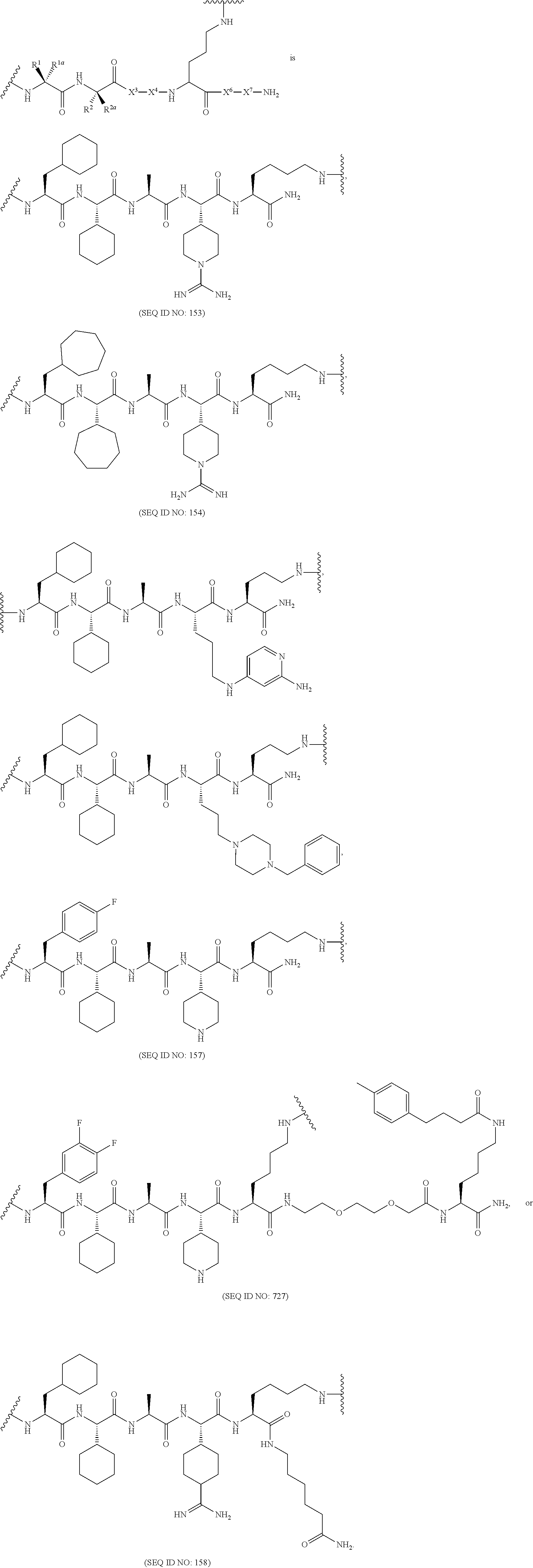

27. The compound of claim 17, wherein: 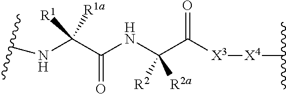
is:

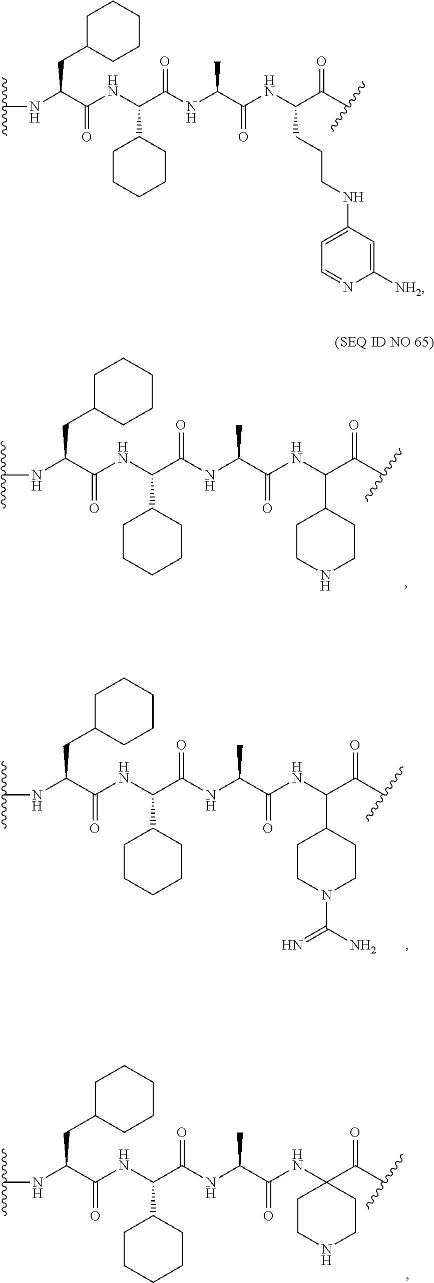 [SMILES: *N[C@@H](CC1CCCCC1)C(=O)N[C@H](C(=O)N[C@@H](C)C(=O)N[C@@H](CCCNc1ccnc(N)c1)C(*)=O)C1CCCCC1 ; *N[C@@H](CC1CCCCC1)C(=O)N[C@H](C(=O)N[C@@H](C)C(=O)NC(C(*)=O)C1CCNCC1)C1CCCCC1 ; *N[C@@H](CC1CCCCC1)C(=O)N[C@H](C(=O)N[C@@H](C)C(=O)NC(C(*)=O)C1CCN(C(=N)N)CC1)C1CCCCC1 ; *N[C@@H](CC1CCCCC1)C(=O)N[C@H](C(=O)N[C@@H](C)C(=O)NC1(C(*)=O)CCNCC1)C1CCCCC1]

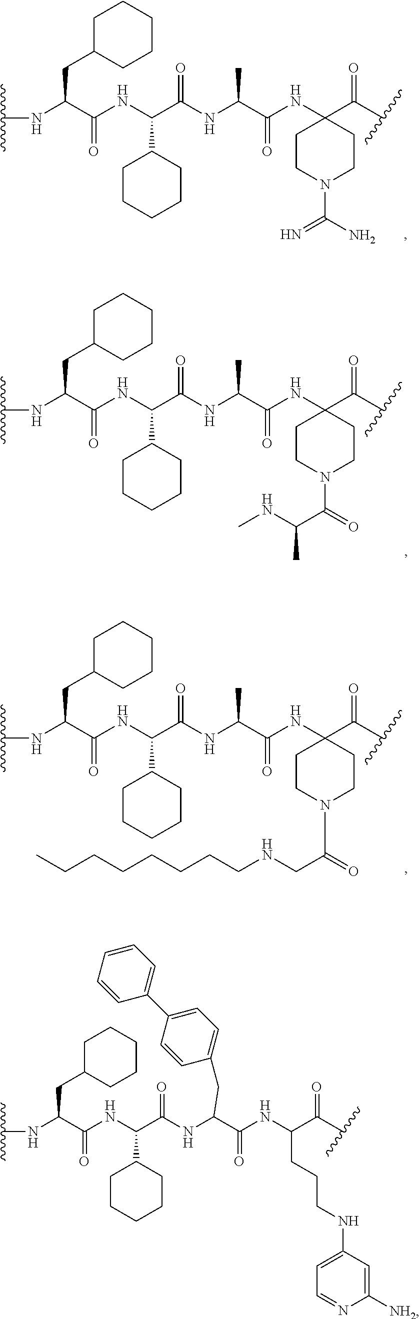 [SMILES: *N[C@@H](CC1CCCCC1)C(=O)N[C@H](C(=O)N[C@@H](C)C(=O)NC1(C(*)=O)CCN(C(=N)N)CC1)C1CCCCC1 ; *N[C@@H](CC1CCCCC1)C(=O)N[C@H](C(=O)N[C@@H](C)C(=O)NC1(C(*)=O)CCN(C(=O)[C@@H](C)NC)CC1)C1CCCCC1 ; *N[C@@H](CC1CCCCC1)C(=O)N[C@H](C(=O)N[C@@H](C)C(=O)NC1(C(*)=O)CCN(C(=O)CNCCCCCCCC)CC1)C1CCCCC1 ; *N[C@@H](CC1CCCCC1)C(=O)N[C@H](C(=O)NC(Cc1ccc(-c2ccccc2)cc1)C(=O)NC(CCCNc1ccnc(N)c1)C(*)=O)C1CCCCC1]

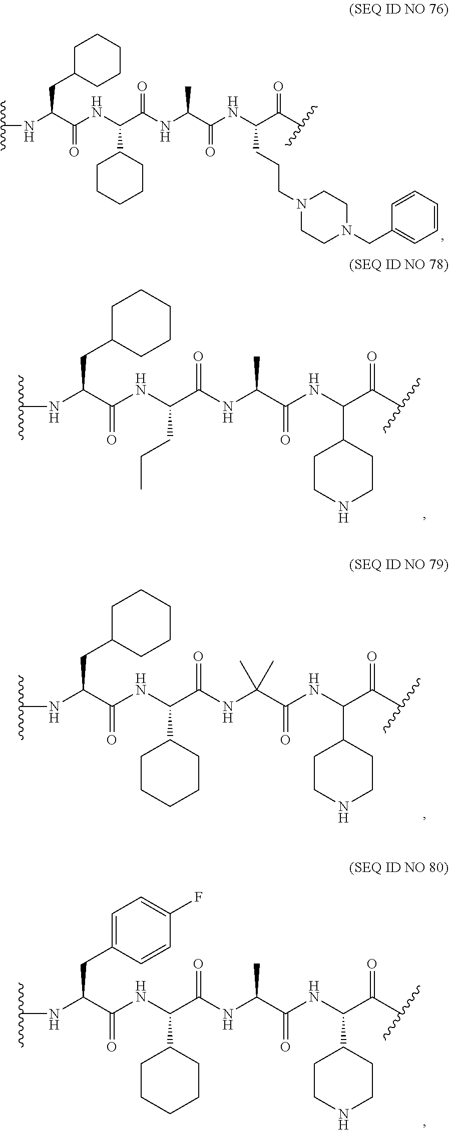 [SMILES: *N[C@@H](CC1CCCCC1)C(=O)N[C@H](C(=O)N[C@@H](C)C(=O)N[C@@H](CCCN1CCN(Cc2ccccc2)CC1)C(*)=O)C1CCCCC1 ; *N[C@@H](CC1CCCCC1)C(=O)N[C@@H](CCC)C(=O)N[C@@H](C)C(=O)NC(C(*)=O)C1CCNCC1 ; *N[C@@H](CC1CCCCC1)C(=O)N[C@H](C(=O)NC(C)(C)C(=O)NC(C(*)=O)C1CCNCC1)C1CCCCC1 ; *N[C@@H](Cc1ccc(F)cc1)C(=O)N[C@H](C(=O)N[C@@H](C)C(=O)N[C@H](C(*)=O)C1CCNCC1)C1CCCCC1]

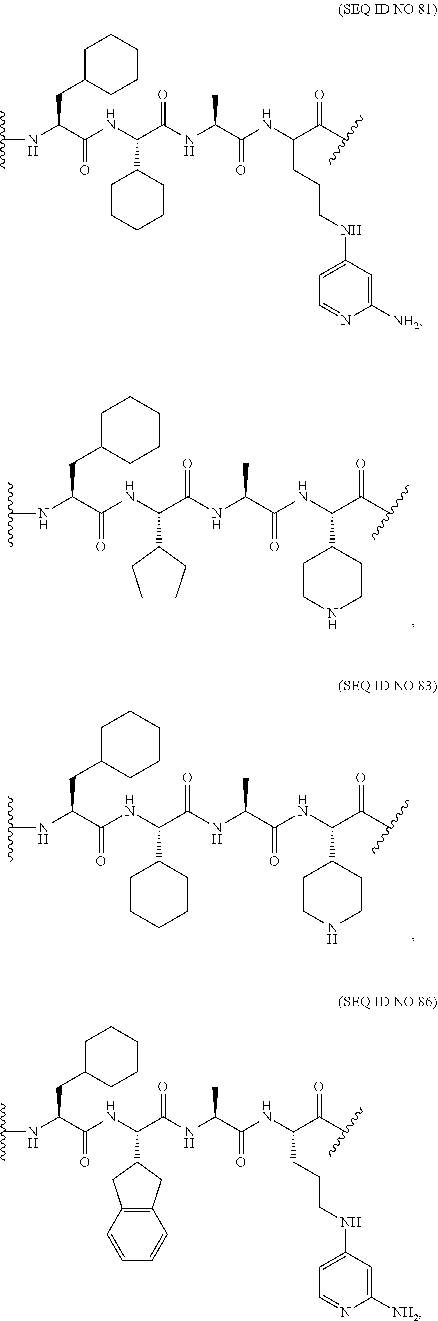 [SMILES: *N[C@@H](CC1CCCCC1)C(=O)N[C@H](C(=O)N[C@@H](C)C(=O)NC(CCCNc1ccnc(N)c1)C(*)=O)C1CCCCC1 ; *N[C@@H](CC1CCCCC1)C(=O)N[C@H](C(=O)N[C@@H](C)C(=O)N[C@H](C(*)=O)C1CCNCC1)C(CC)CC ; *N[C@@H](CC1CCCCC1)C(=O)N[C@H](C(=O)N[C@@H](C)C(=O)N[C@H](C(*)=O)C1CCNCC1)C1CCCCC1 ; *N[C@@H](CC1CCCCC1)C(=O)N[C@H](C(=O)N[C@@H](C)C(=O)N[C@@H](CCCNc1ccnc(N)c1)C(*)=O)C1Cc2ccccc2C1]

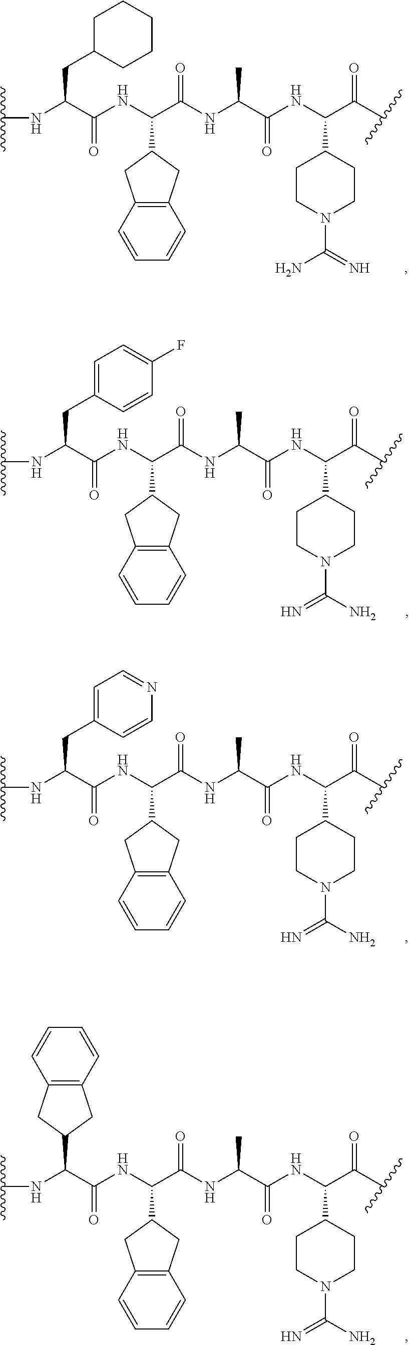 [SMILES: *N[C@@H](CC1CCCCC1)C(=O)N[C@H](C(=O)N[C@@H](C)C(=O)N[C@H](C(*)=O)C1CCN(C(=N)N)CC1)C1Cc2ccccc2C1 ; *N[C@@H](Cc1ccc(F)cc1)C(=O)N[C@H](C(=O)N[C@@H](C)C(=O)N[C@H](C(*)=O)C1CCN(C(=N)N)CC1)C1Cc2ccccc2C1 ; *N[C@@H](Cc1ccncc1)C(=O)N[C@H](C(=O)N[C@@H](C)C(=O)N[C@H](C(*)=O)C1CCN(C(=N)N)CC1)C1Cc2ccccc2C1 ; *N[C@H](C(=O)N[C@H](C(=O)N[C@@H](C)C(=O)N[C@H](C(*)=O)C1CCN(C(=N)N)CC1)C1Cc2ccccc2C1)C1Cc2ccccc2C1]

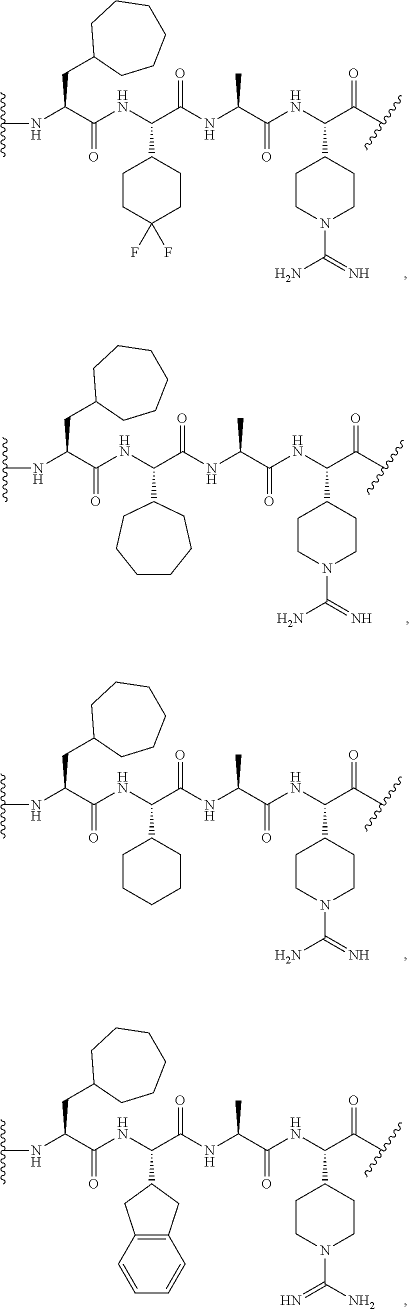 [SMILES: *N[C@@H](CC1CCCCCC1)C(=O)N[C@H](C(=O)N[C@@H](C)C(=O)N[C@H](C(*)=O)C1CCN(C(=N)N)CC1)C1CCC(F)(F)CC1 ; *N[C@@H](CC1CCCCCC1)C(=O)N[C@H](C(=O)N[C@@H](C)C(=O)N[C@H](C(*)=O)C1CCN(C(=N)N)CC1)C1CCCCCC1 ; *N[C@@H](CC1CCCCCC1)C(=O)N[C@H](C(=O)N[C@@H](C)C(=O)N[C@H](C(*)=O)C1CCN(C(=N)N)CC1)C1CCCCC1 ; *N[C@@H](CC1CCCCCC1)C(=O)N[C@H](C(=O)N[C@@H](C)C(=O)N[C@H](C(*)=O)C1CCN(C(=N)N)CC1)C1Cc2ccccc2C1]

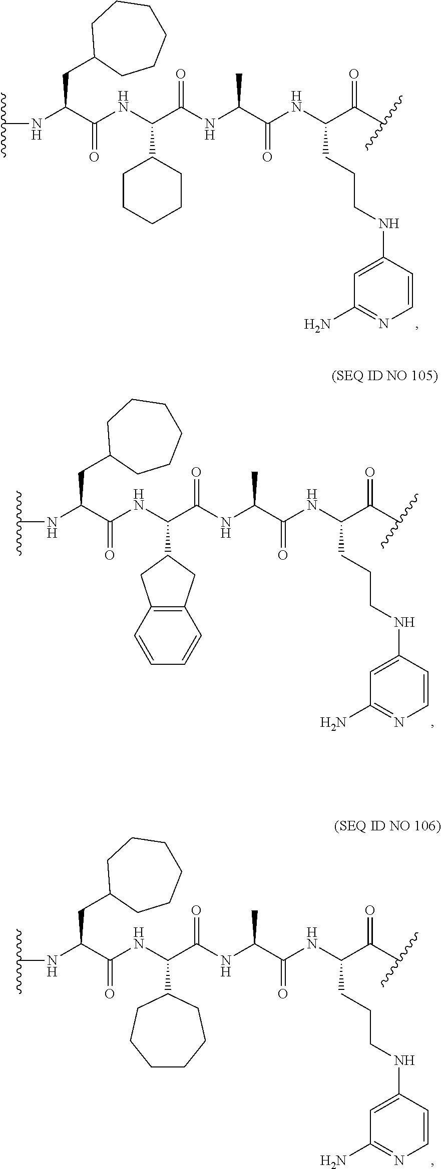 [SMILES: *N[C@@H](CC1CCCCCC1)C(=O)N[C@H](C(=O)N[C@@H](C)C(=O)N[C@@H](CCCNc1ccnc(N)c1)C(*)=O)C1CCCCC1 ; *N[C@@H](CC1CCCCCC1)C(=O)N[C@H](C(=O)N[C@@H](C)C(=O)N[C@@H](CCCNc1ccnc(N)c1)C(*)=O)C1Cc2ccccc2C1 ; *N[C@@H](CC1CCCCCC1)C(=O)N[C@H](C(=O)N[C@@H](C)C(=O)N[C@@H](CCCNc1ccnc(N)c1)C(*)=O)C1CCCCCC1]

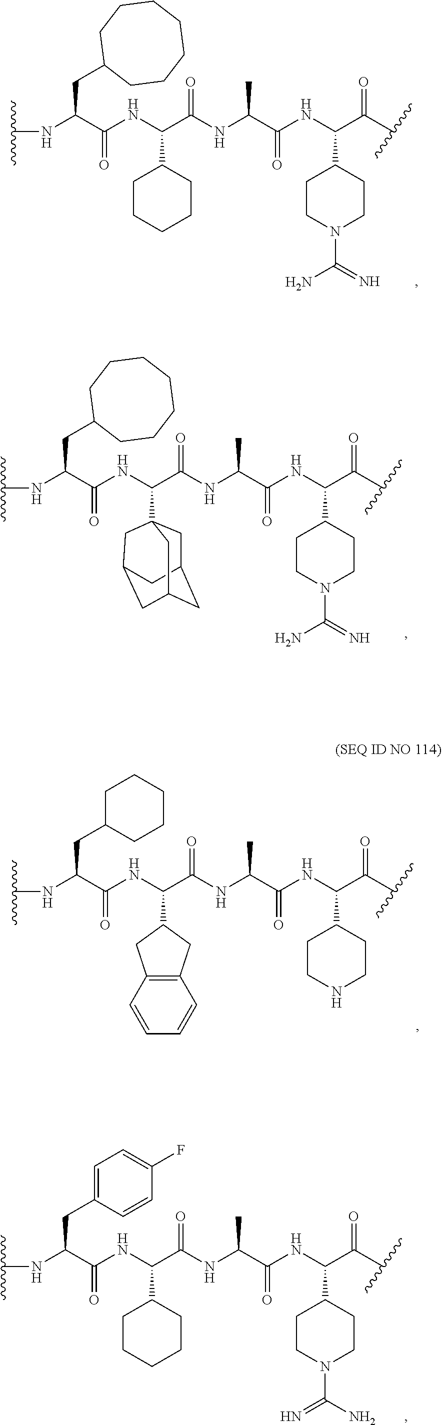 [SMILES: *N[C@@H](CC1CCCCCCC1)C(=O)N[C@H](C(=O)N[C@@H](C)C(=O)N[C@H](C(*)=O)C1CCN(C(=N)N)CC1)C1CCCCC1 ; *N[C@@H](CC1CCCCCCC1)C(=O)N[C@H](C(=O)N[C@@H](C)C(=O)N[C@H](C(*)=O)C1CCN(C(=N)N)CC1)C12CC3CC(CC(C3)C1)C2 ; *N[C@@H](CC1CCCCC1)C(=O)N[C@H](C(=O)N[C@@H](C)C(=O)N[C@H](C(*)=O)C1CCNCC1)C1Cc2ccccc2C1 ; *N[C@@H](Cc1ccc(F)cc1)C(=O)N[C@H](C(=O)N[C@@H](C)C(=O)N[C@H](C(*)=O)C1CCN(C(=N)N)CC1)C1CCCCC1]

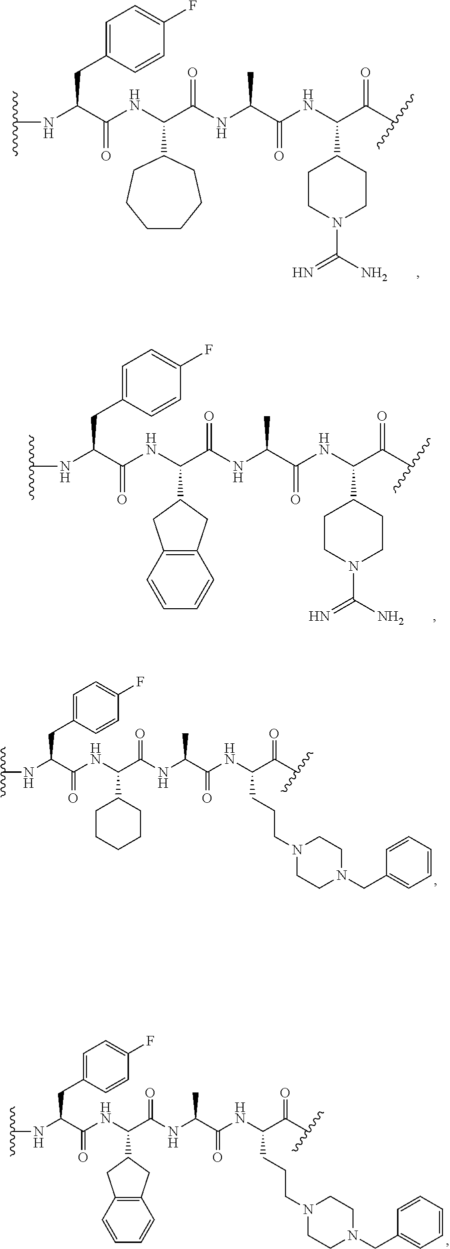 [SMILES: *N[C@@H](Cc1ccc(F)cc1)C(=O)N[C@H](C(=O)N[C@@H](C)C(=O)N[C@H](C(*)=O)C1CCN(C(=N)N)CC1)C1CCCCCC1 ; *N[C@@H](Cc1ccc(F)cc1)C(=O)N[C@H](C(=O)N[C@@H](C)C(=O)N[C@H](C(*)=O)C1CCN(C(=N)N)CC1)C1Cc2ccccc2C1 ; *N[C@@H](Cc1ccc(F)cc1)C(=O)N[C@H](C(=O)N[C@@H](C)C(=O)N[C@@H](CCCN1CCN(Cc2ccccc2)CC1)C(*)=O)C1CCCCC1 ; *N[C@@H](Cc1ccc(F)cc1)C(=O)N[C@H](C(=O)N[C@@H](C)C(=O)N[C@@H](CCCN1CCN(Cc2ccccc2)CC1)C(*)=O)C1Cc2ccccc2C1]

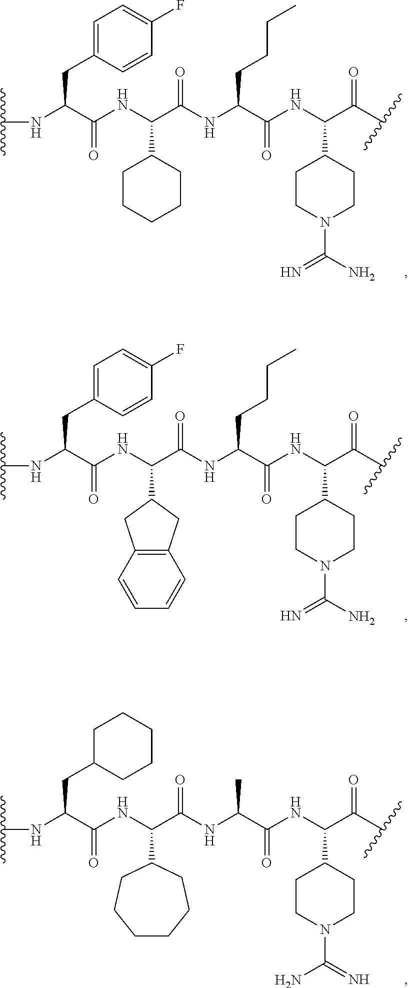 [SMILES: *N[C@@H](Cc1ccc(F)cc1)C(=O)N[C@H](C(=O)N[C@@H](CCCC)C(=O)N[C@H](C(*)=O)C1CCN(C(=N)N)CC1)C1CCCCC1 ; *N[C@@H](Cc1ccc(F)cc1)C(=O)N[C@H](C(=O)N[C@@H](CCCC)C(=O)N[C@H](C(*)=O)C1CCN(C(=N)N)CC1)C1Cc2ccccc2C1 ; *N[C@@H](CC1CCCCC1)C(=O)N[C@H](C(=O)N[C@@H](C)C(=O)N[C@H](C(*)=O)C1CCN(C(=N)N)CC1)C1CCCCCC1]

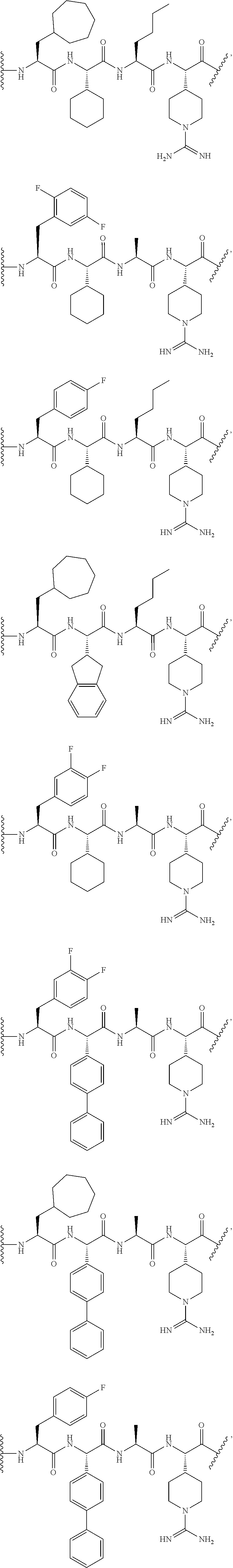

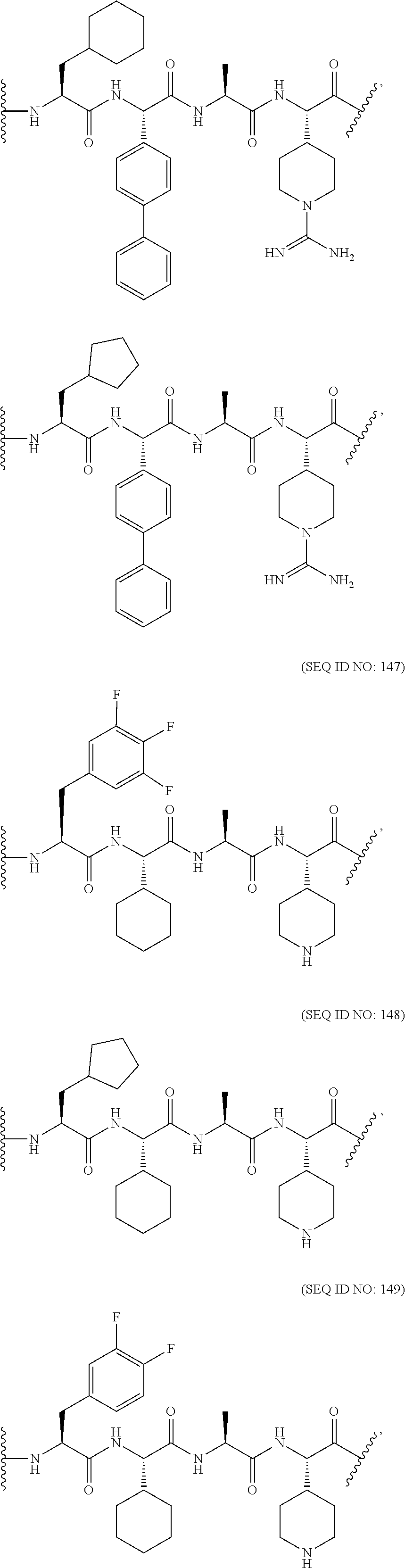

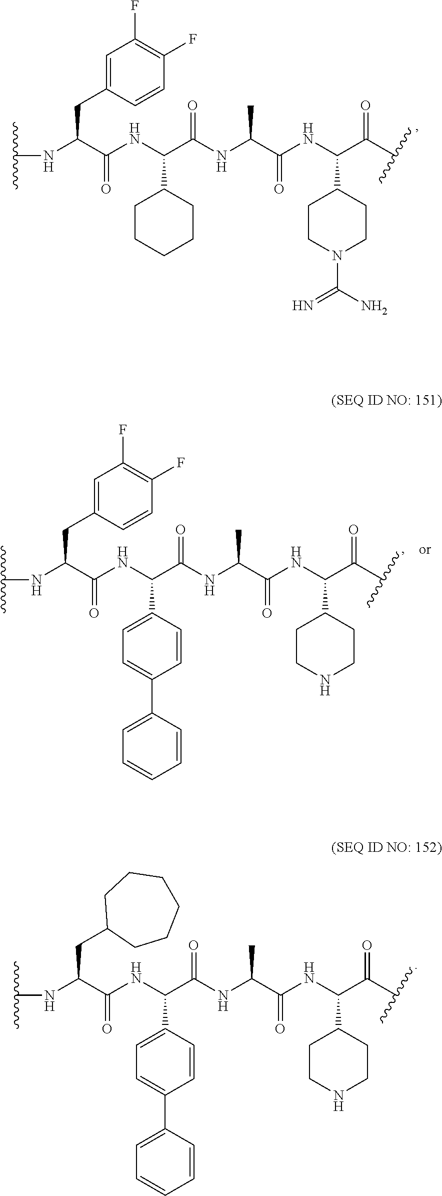 [SMILES: *N[C@@H](Cc1ccc(F)c(F)c1)C(=O)N[C@H](C(=O)N[C@@H](C)C(=O)N[C@H](C(*)=O)C1CCN(C(=N)N)CC1)C1CCCCC1 ; *N[C@@H](Cc1ccc(F)c(F)c1)C(=O)N[C@H](C(=O)N[C@@H](C)C(=O)N[C@H](C(*)=O)C1CCNCC1)c1ccc(-c2ccccc2)cc1 ; *N[C@@H](CC1CCCCCC1)C(=O)N[C@H](C(=O)N[C@@H](C)C(=O)N[C@H](C(*)=O)C1CCNCC1)c1ccc(-c2ccccc2)cc1]

28. The compound of claim 17, wherein:
-L⁶- and -L⁷- are each independently selected from the group consisting of: —NH—C₁-C₆ alkylene-C(═O)—, —NH—(CH₂CH₂O)_{y}—C₁-C₆ alkylene-C(═O)—, —NH(CH₂CH₂NR¹⁶)_{y}—C₁-C₆ alkylene-C(═O)—, —NH—C₁-C₆ alkylene-(unsubstituted piperidinylene)-C(═O)—, —NH-(unsubstituted piperidinylene)-C₁-C₆ alkylene-C(═O)—, —NH—C₁-C₆ alkylene-(unsubstituted cyclohexylene)-C(═O)—, and —NH-(unsubstituted cyclohexylene)-C₁-C₆ alkylene-C(═O)—.

29. The compound of claim 17, wherein:
-L⁶- and -L⁷- are each independently selected from the group consisting of:

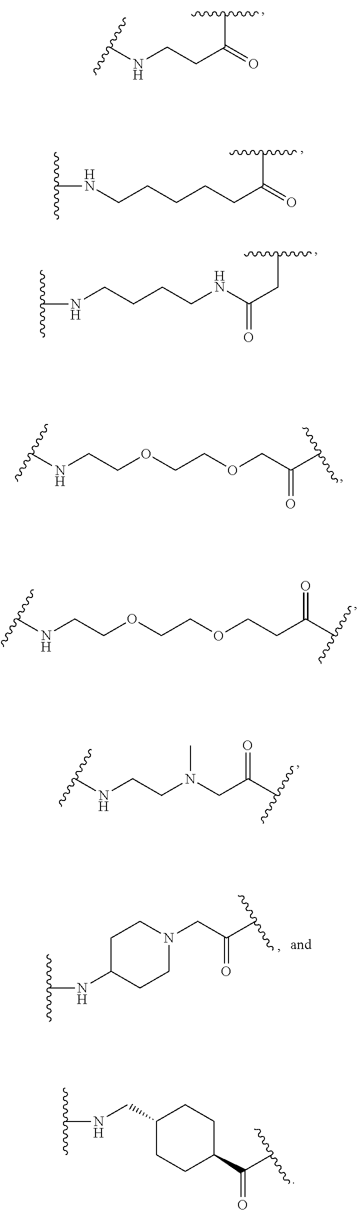

30. A compound selected from: 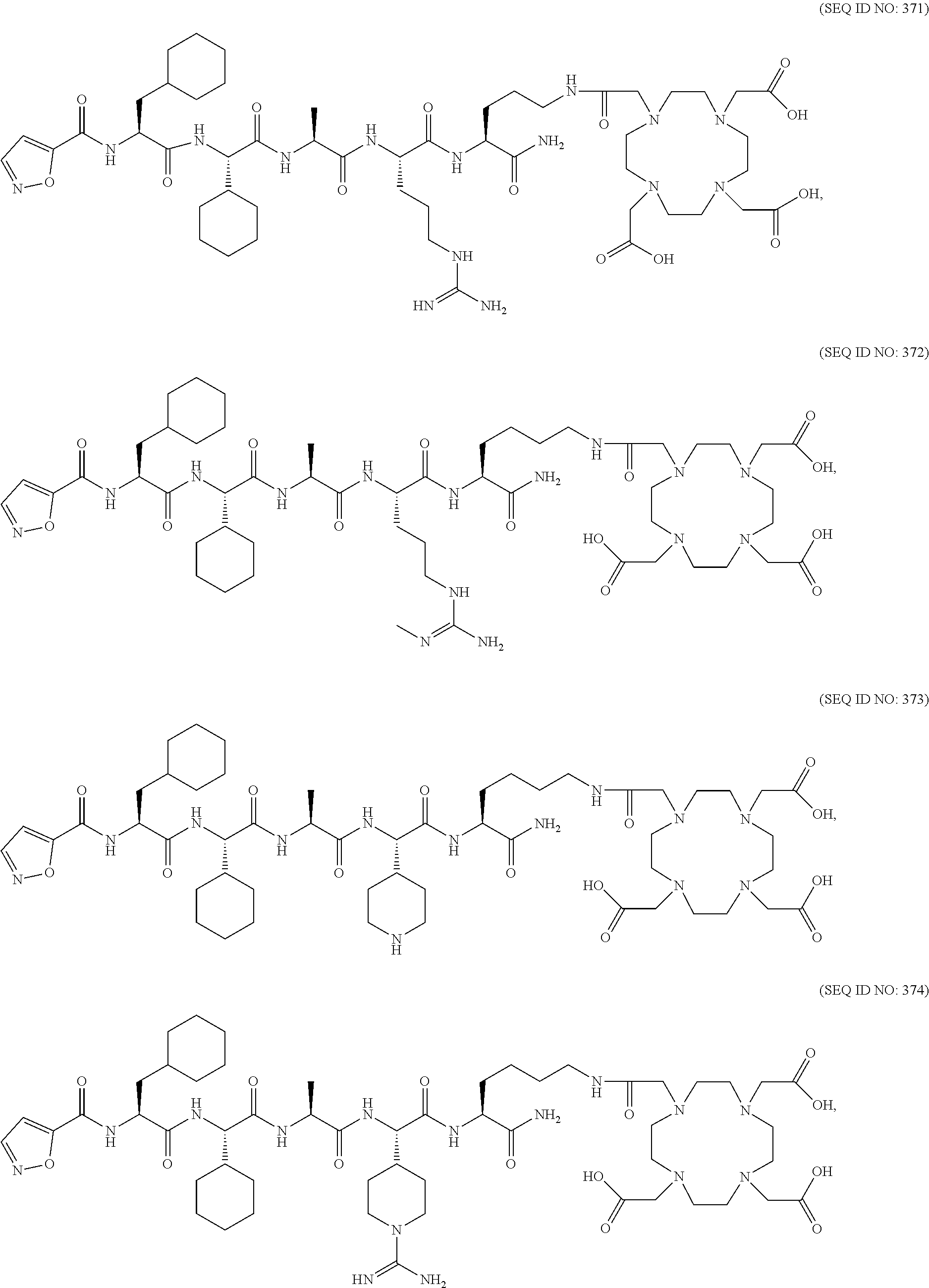 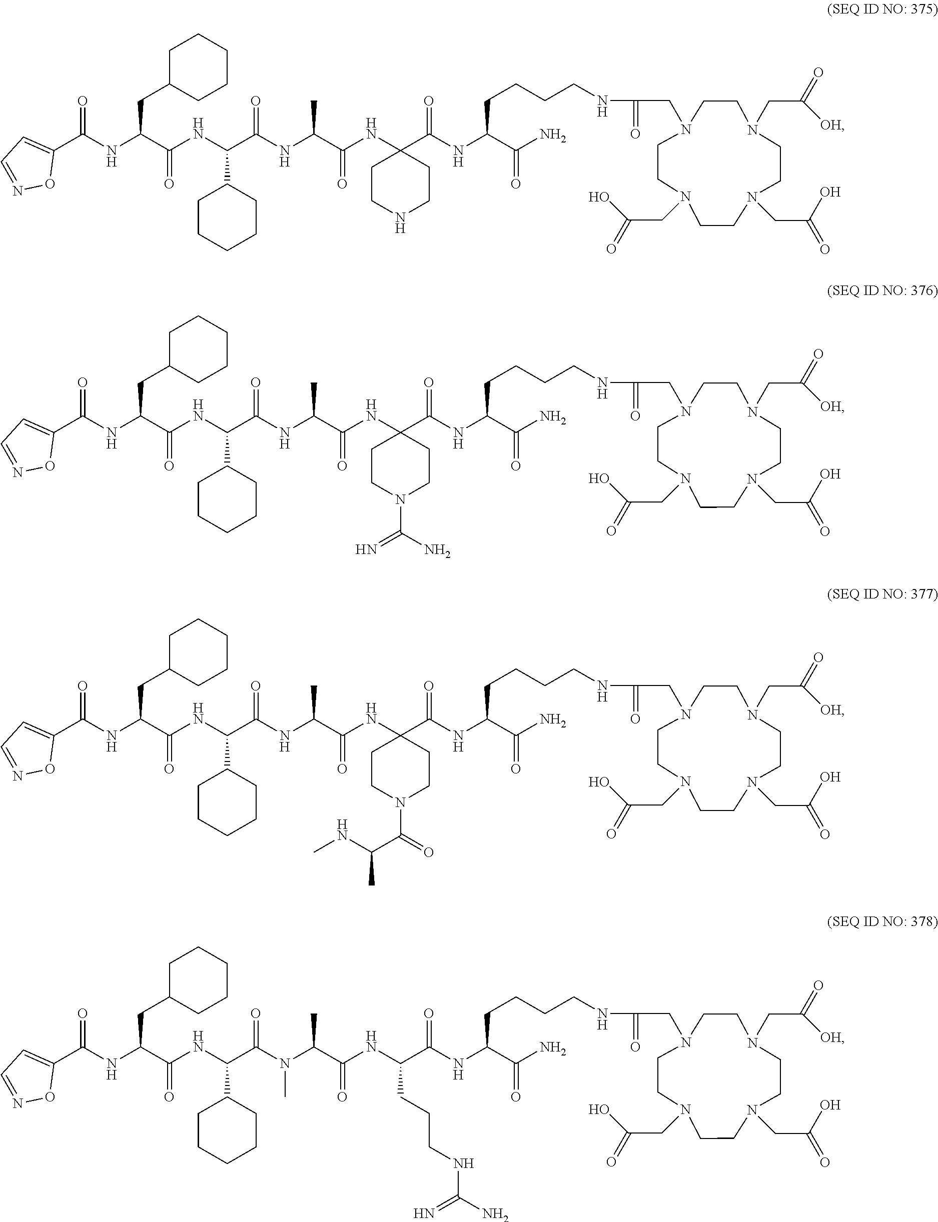 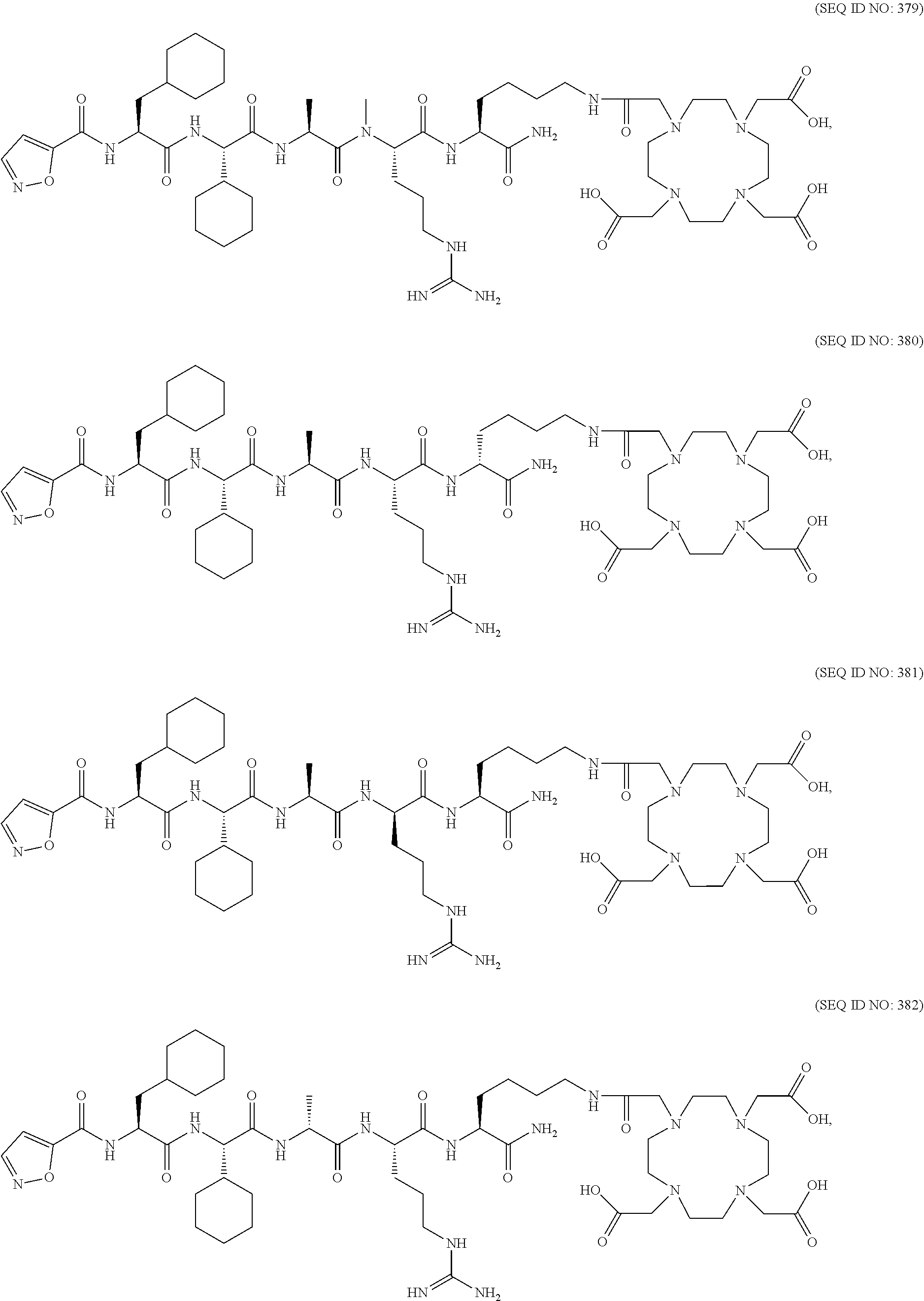 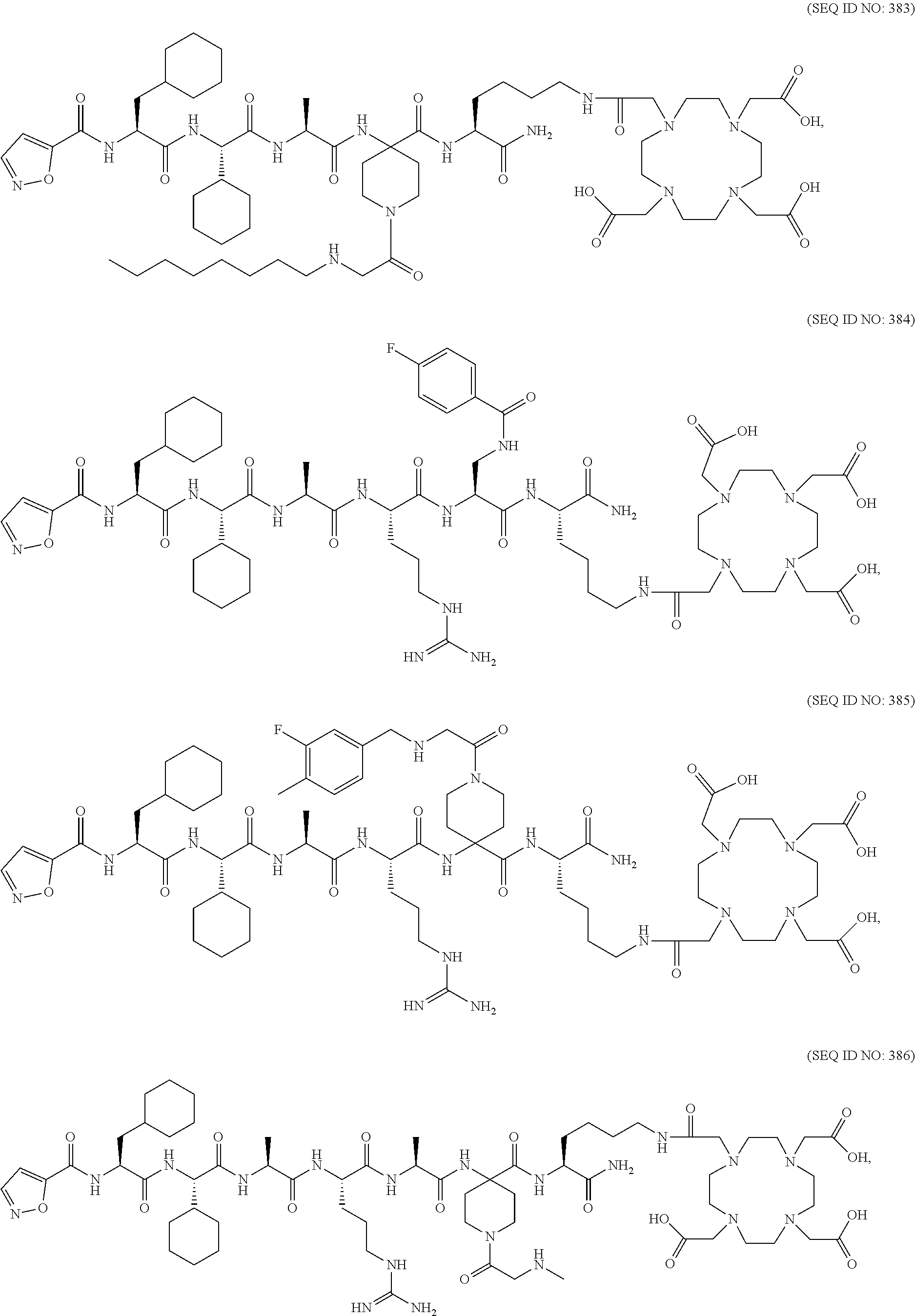 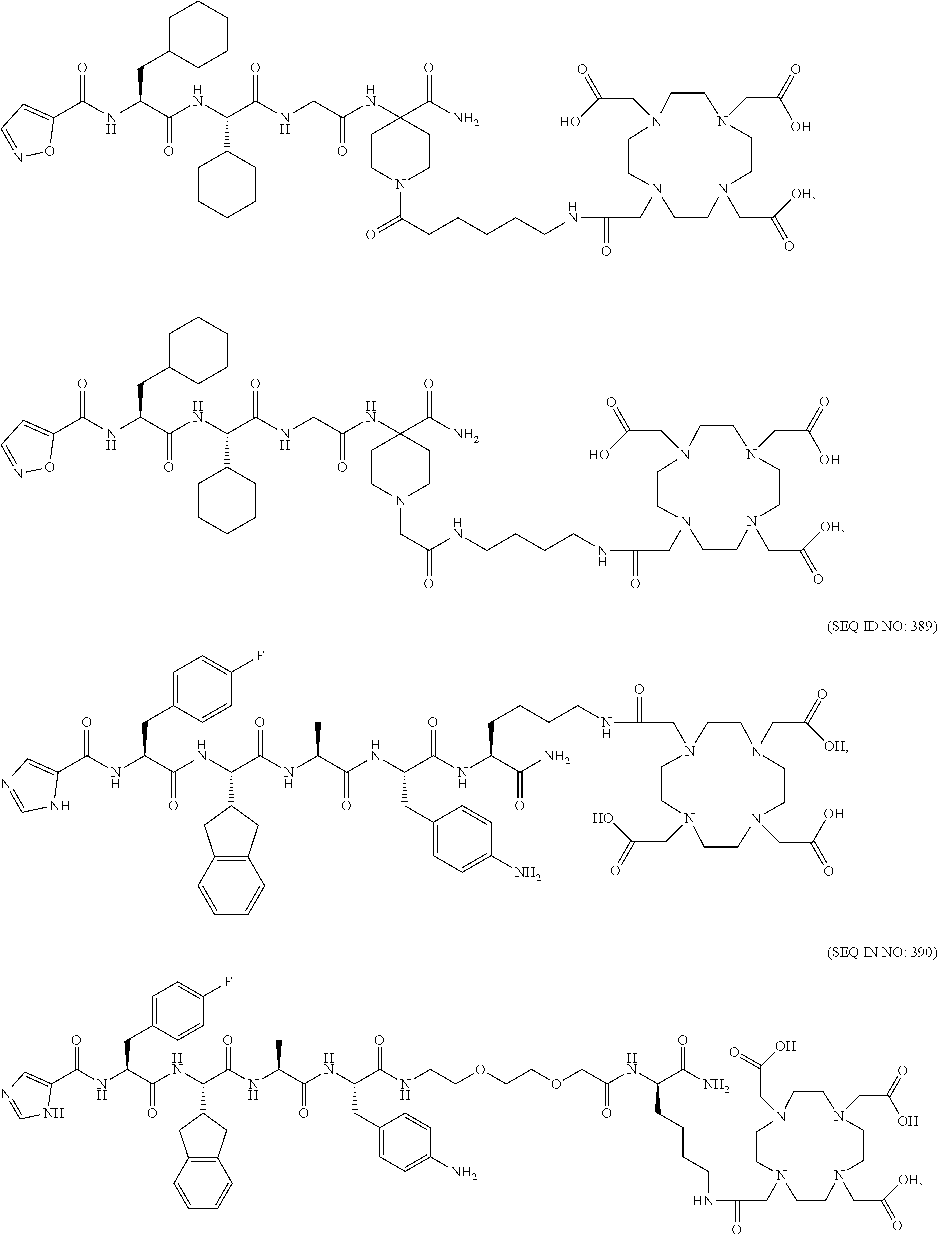 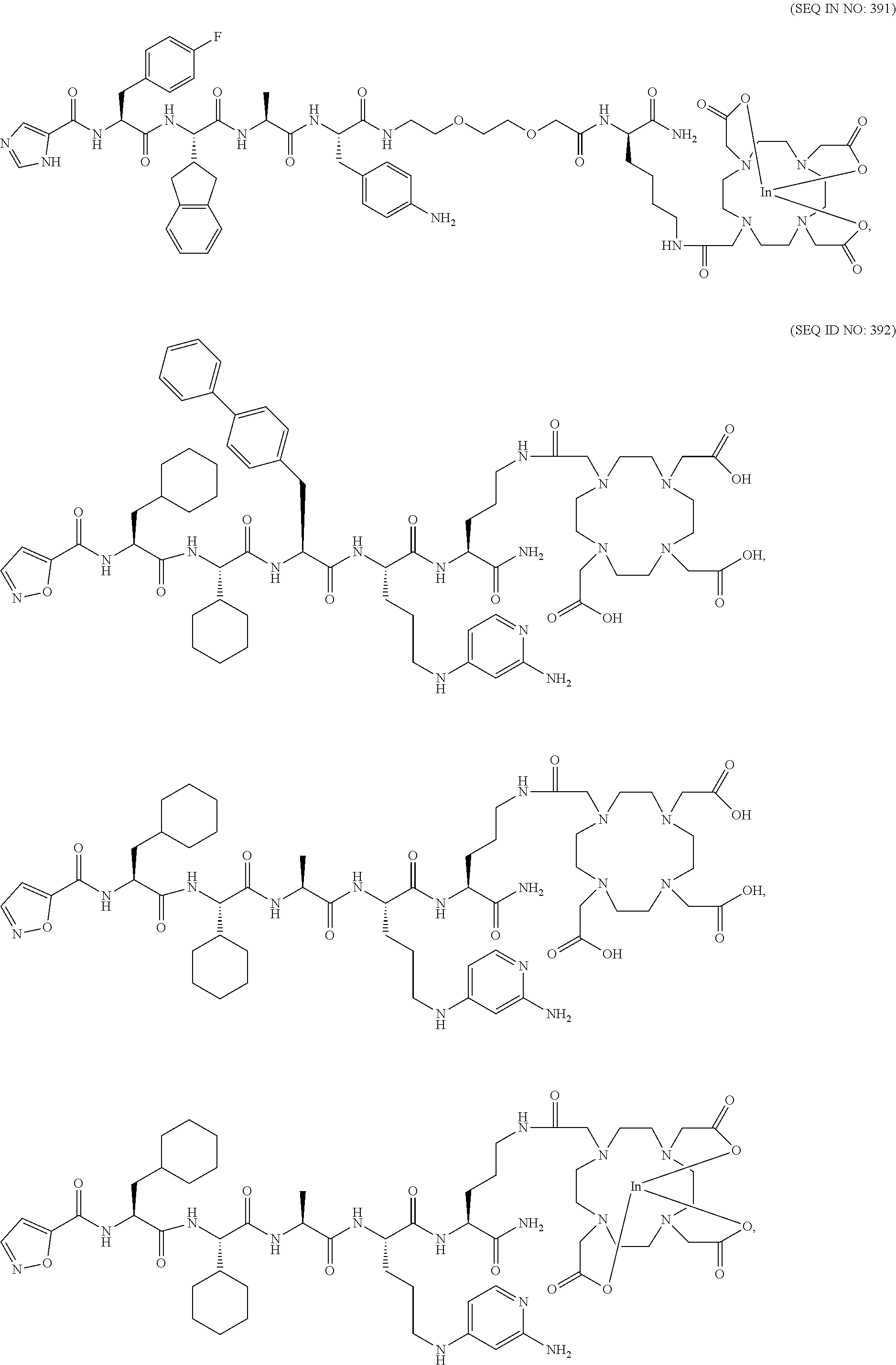 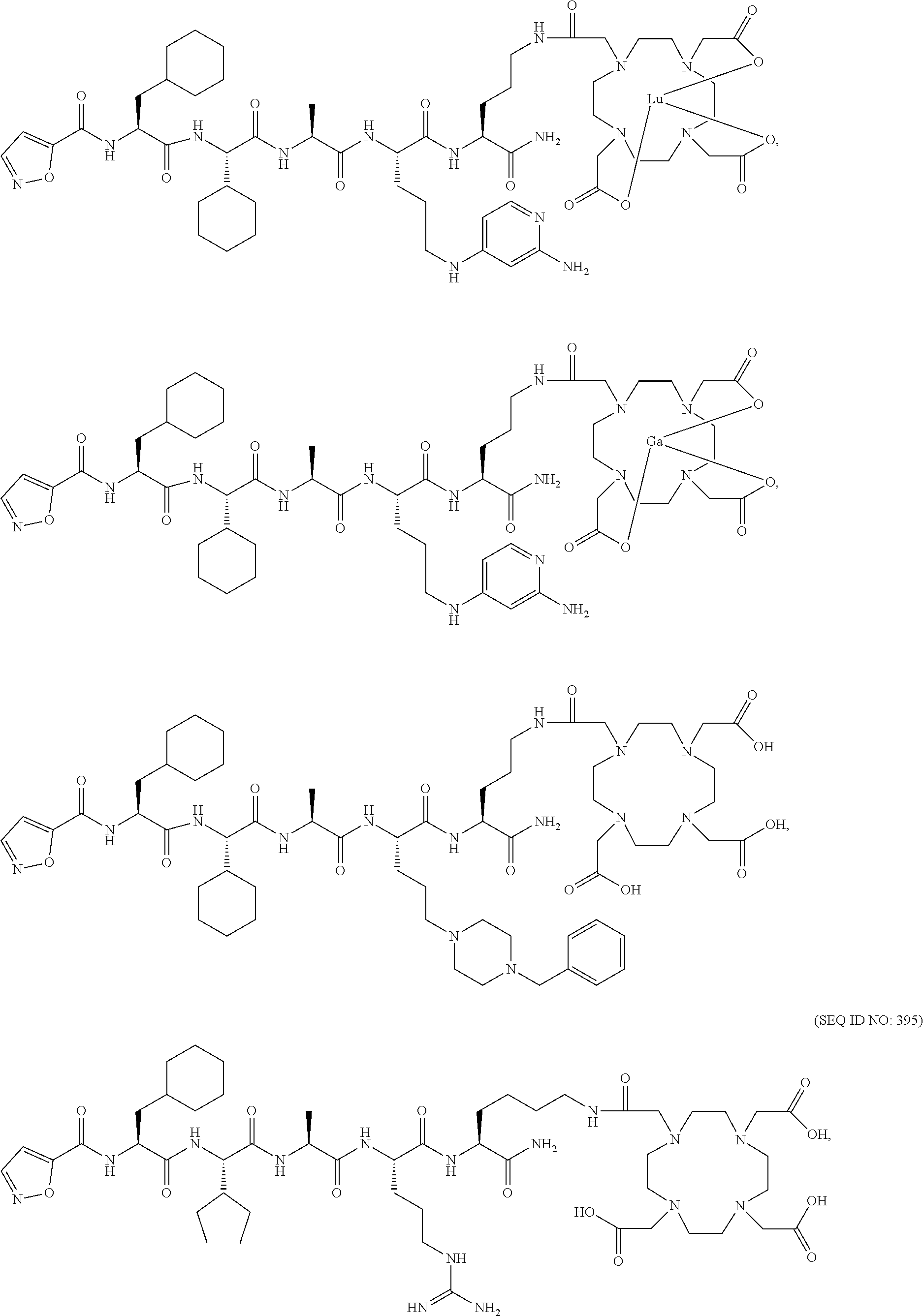 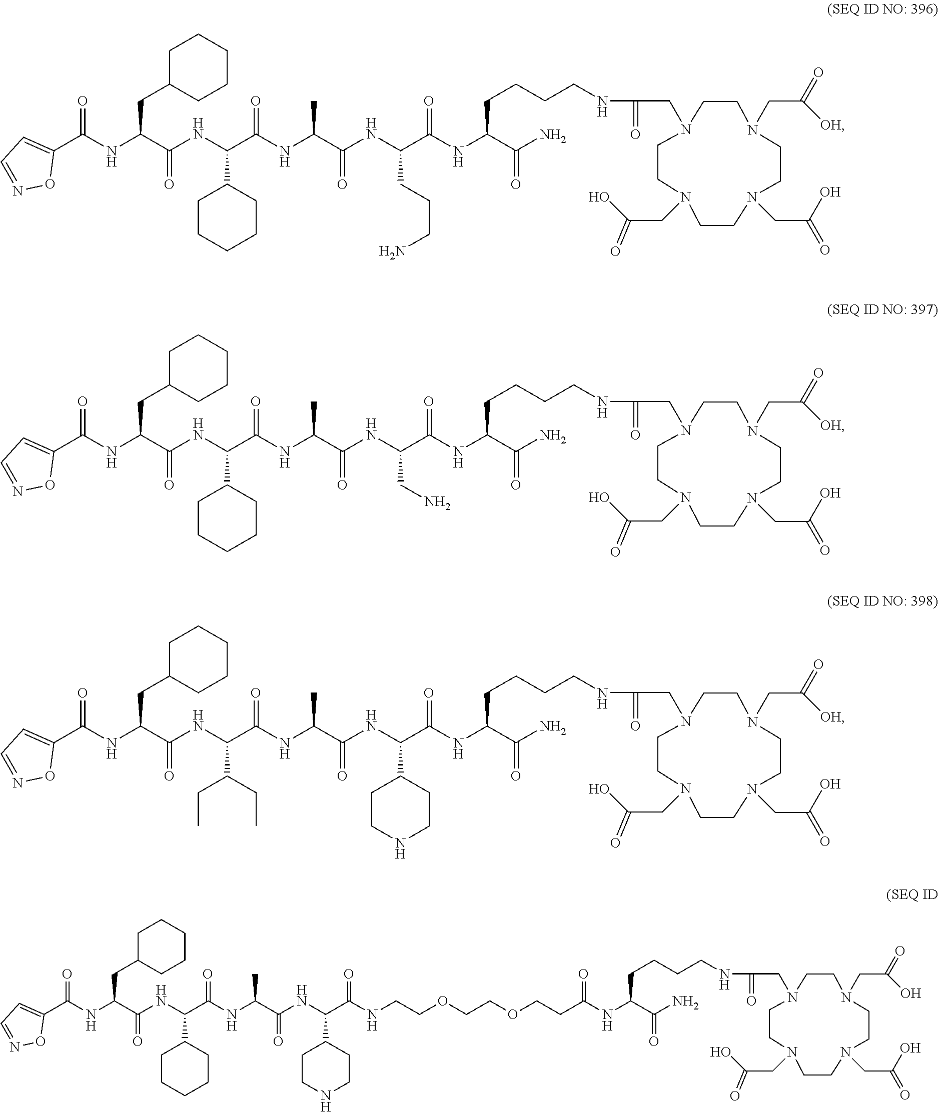 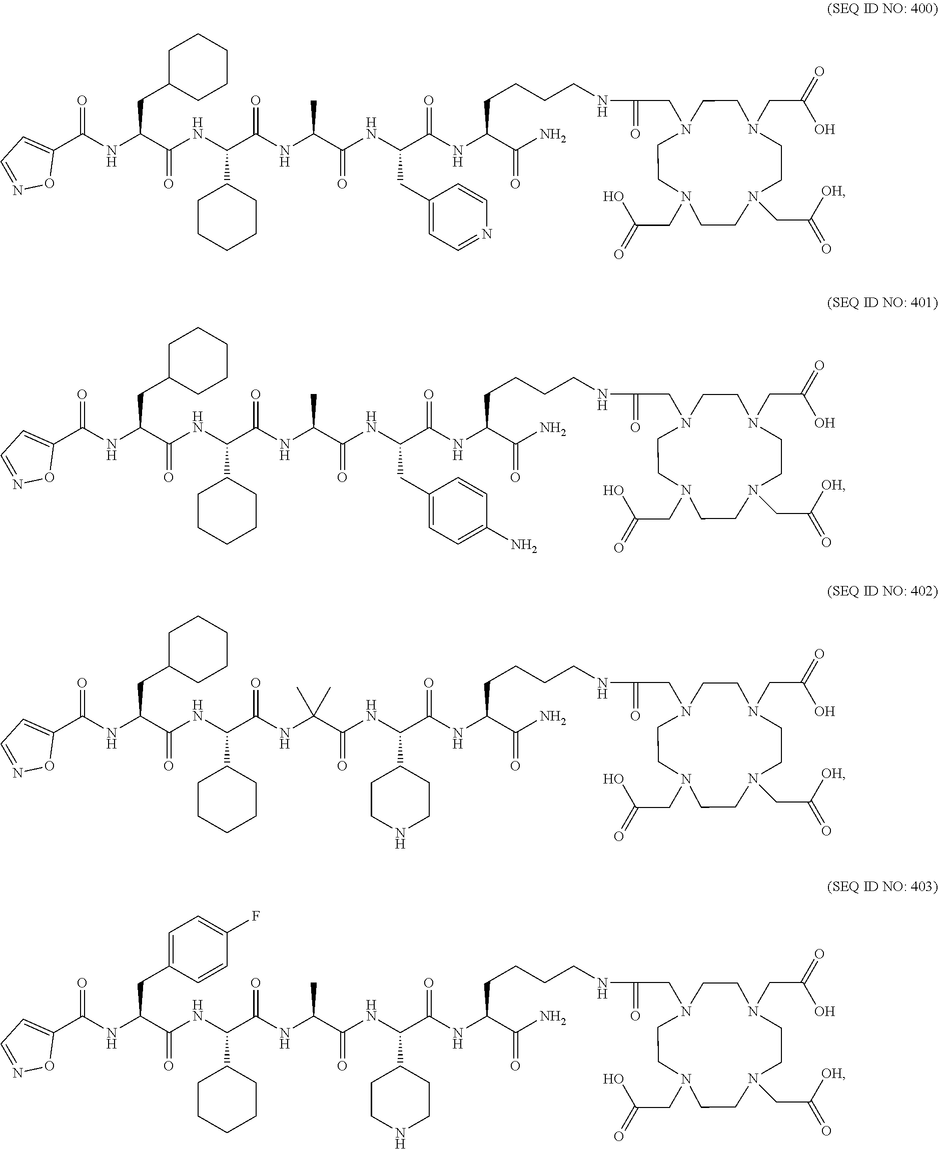 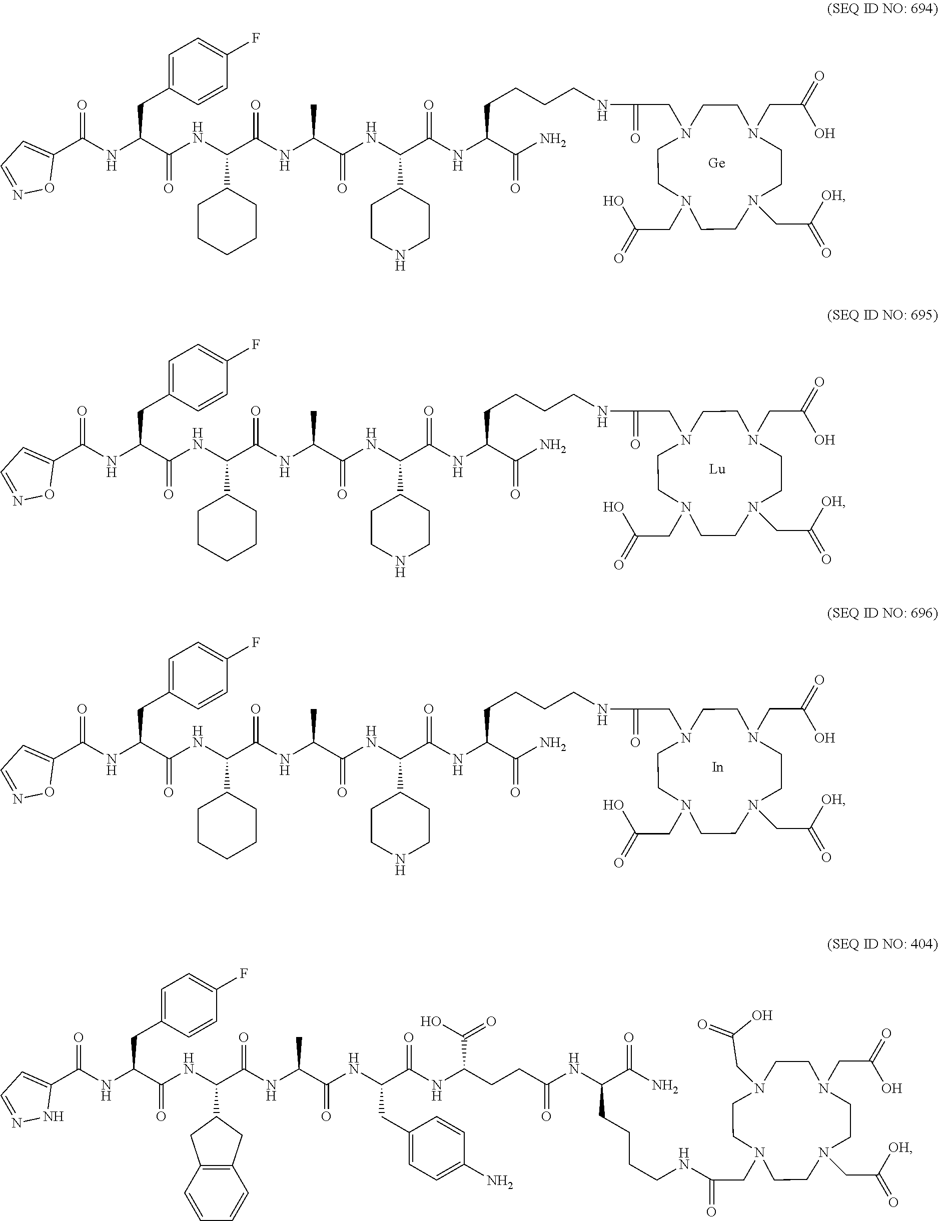 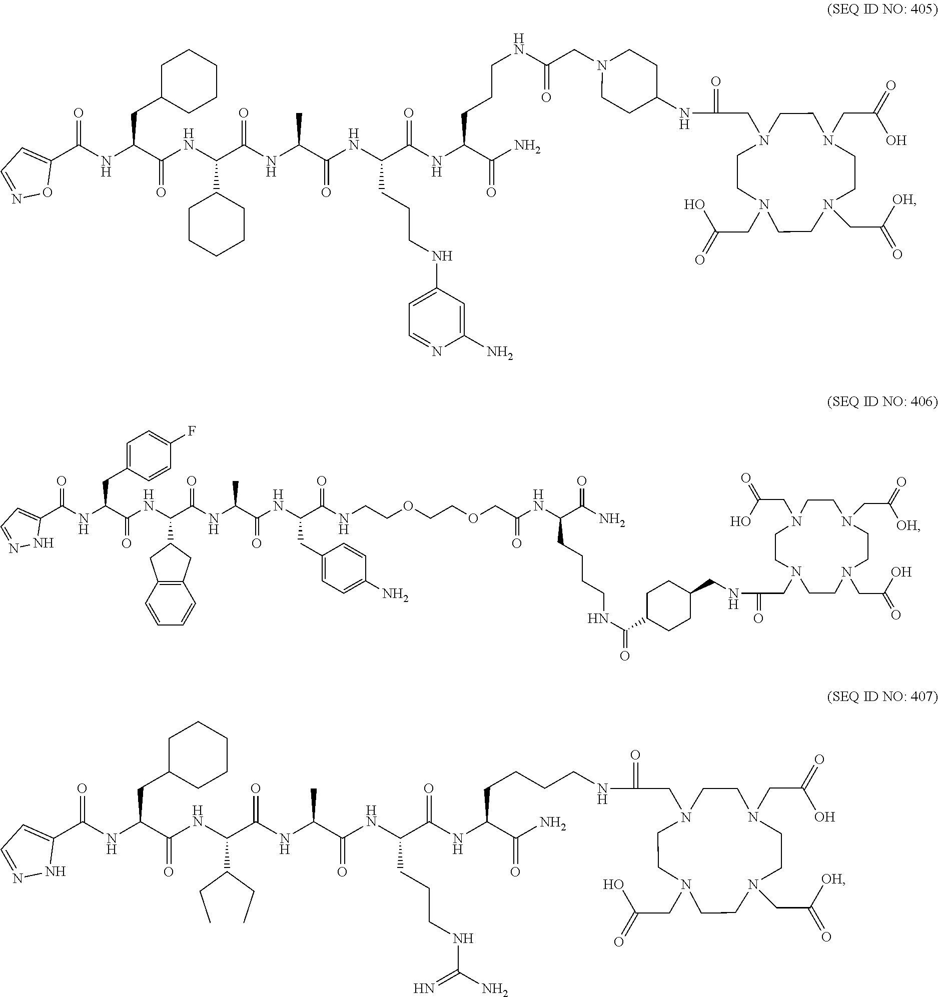 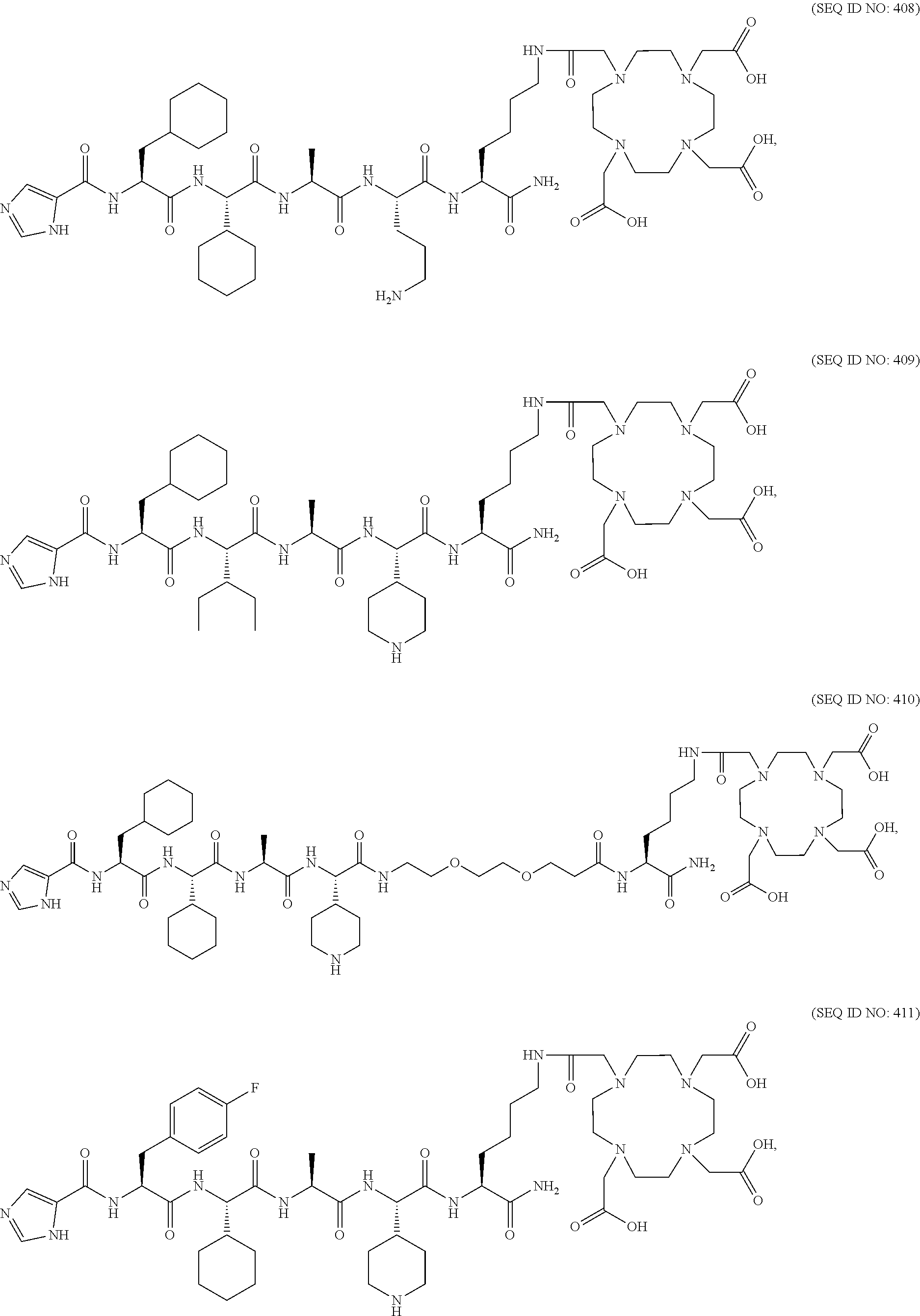 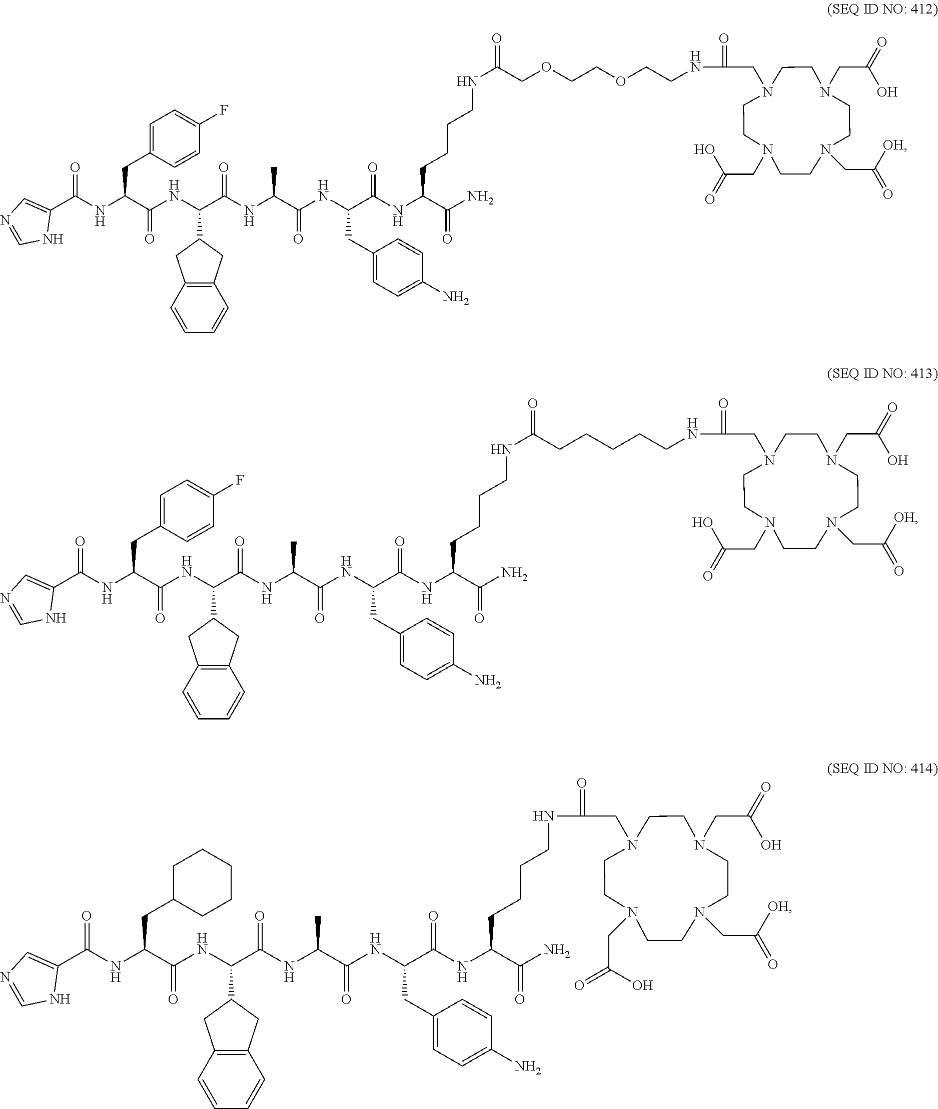 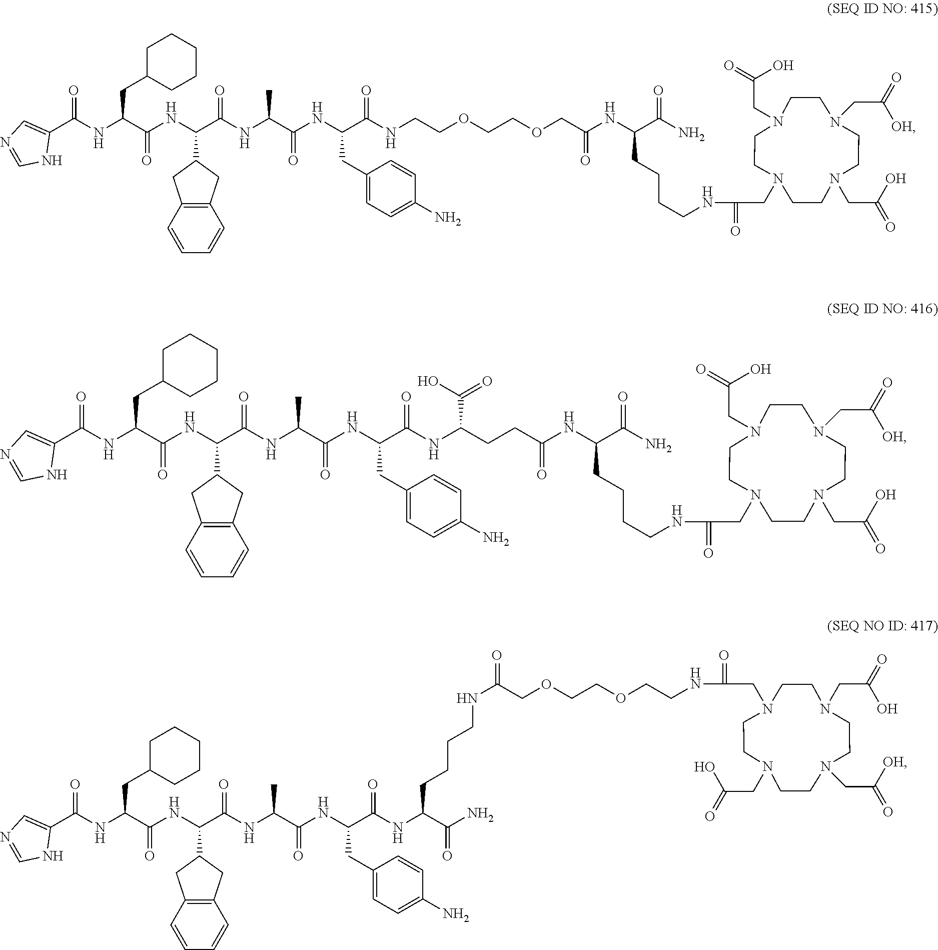 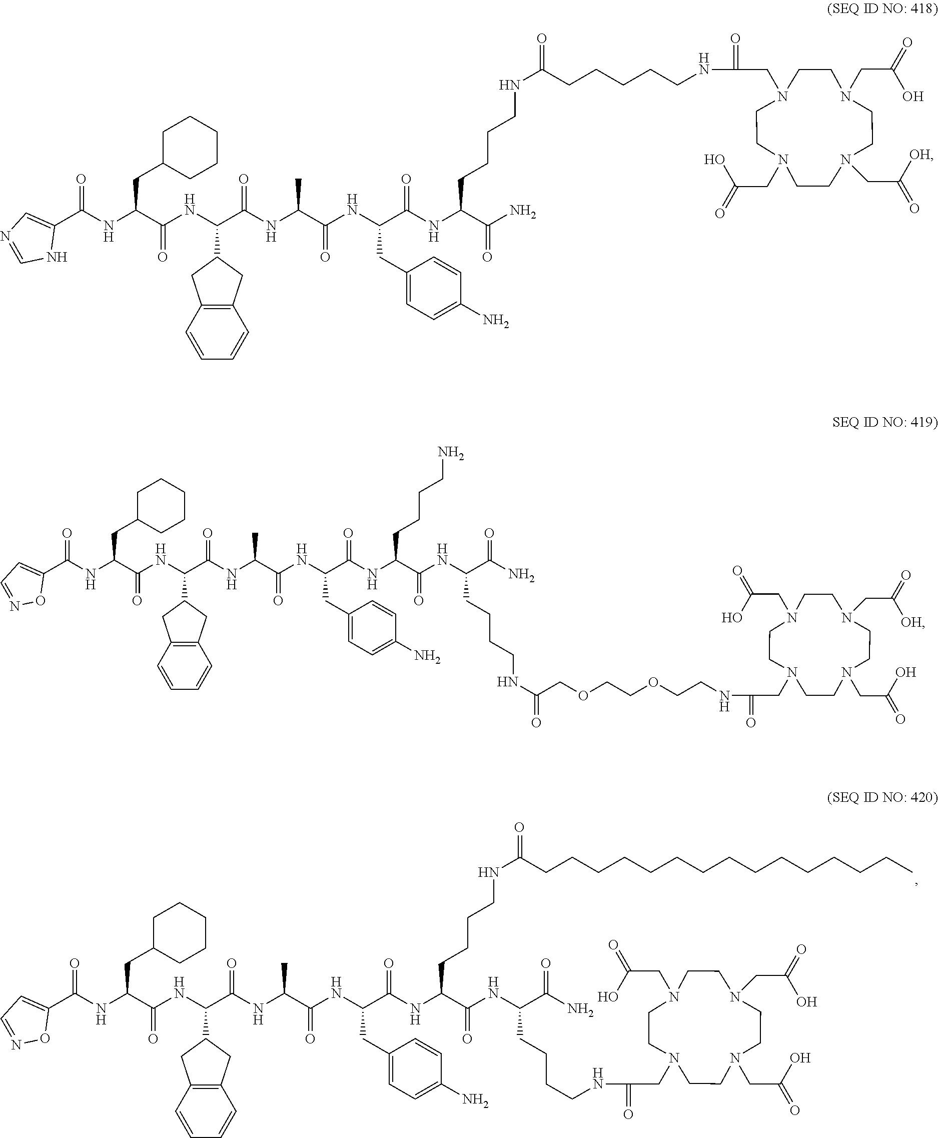 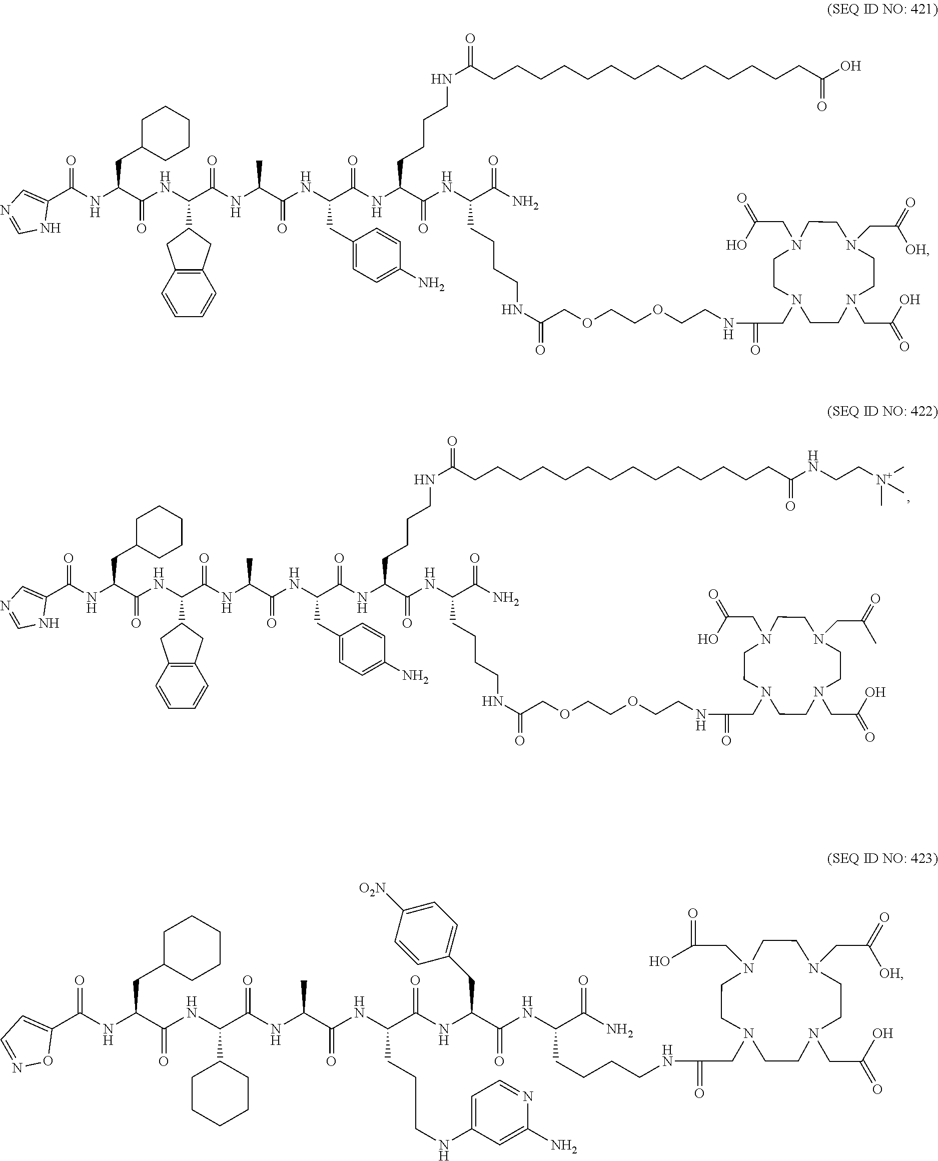 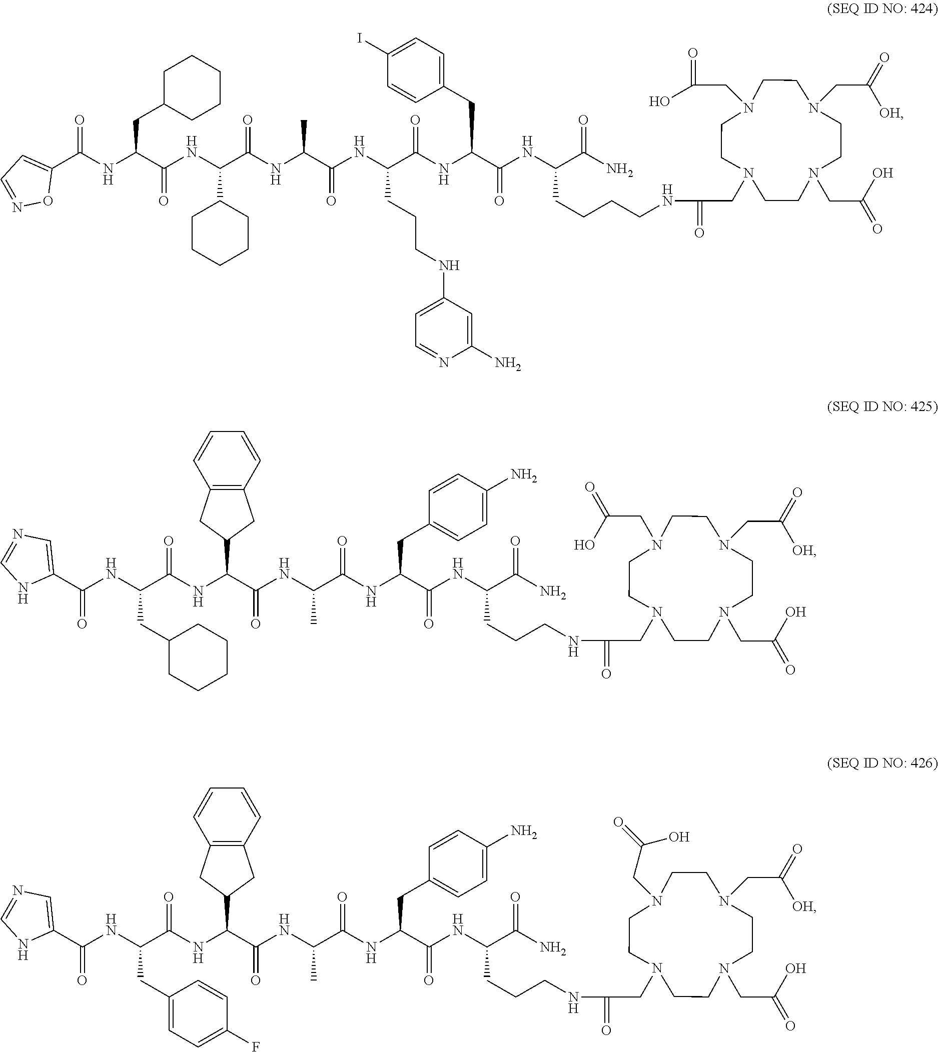 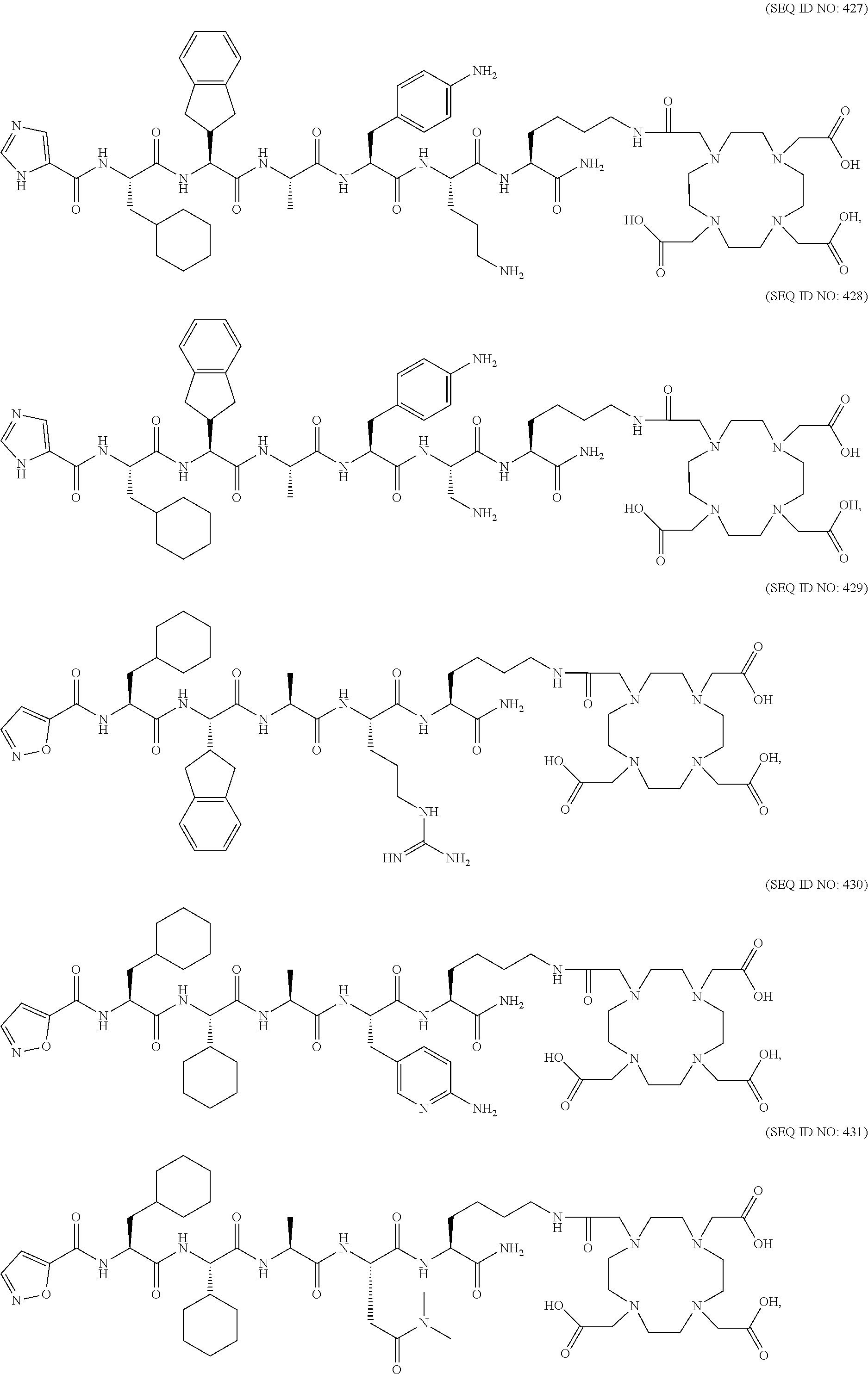 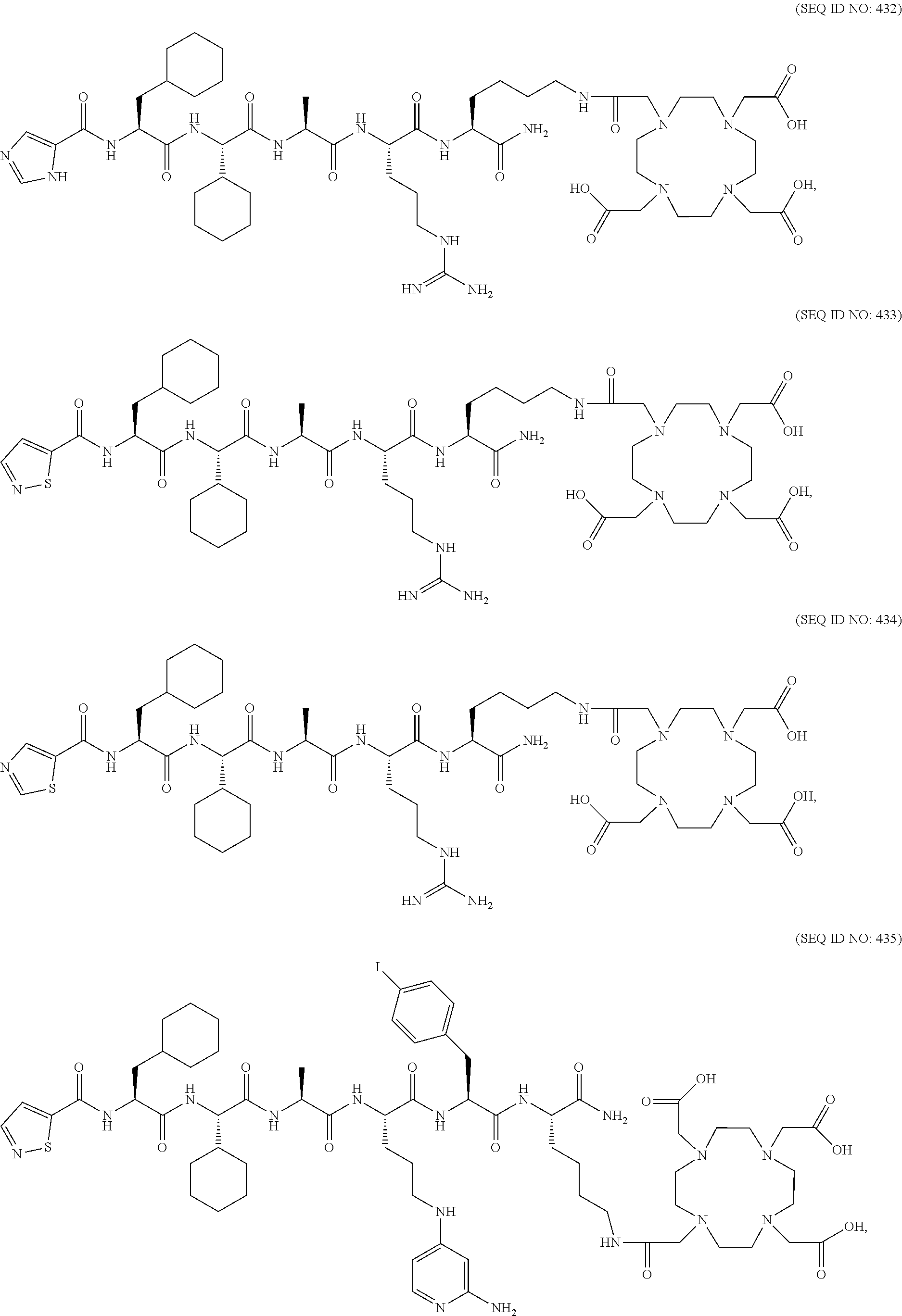 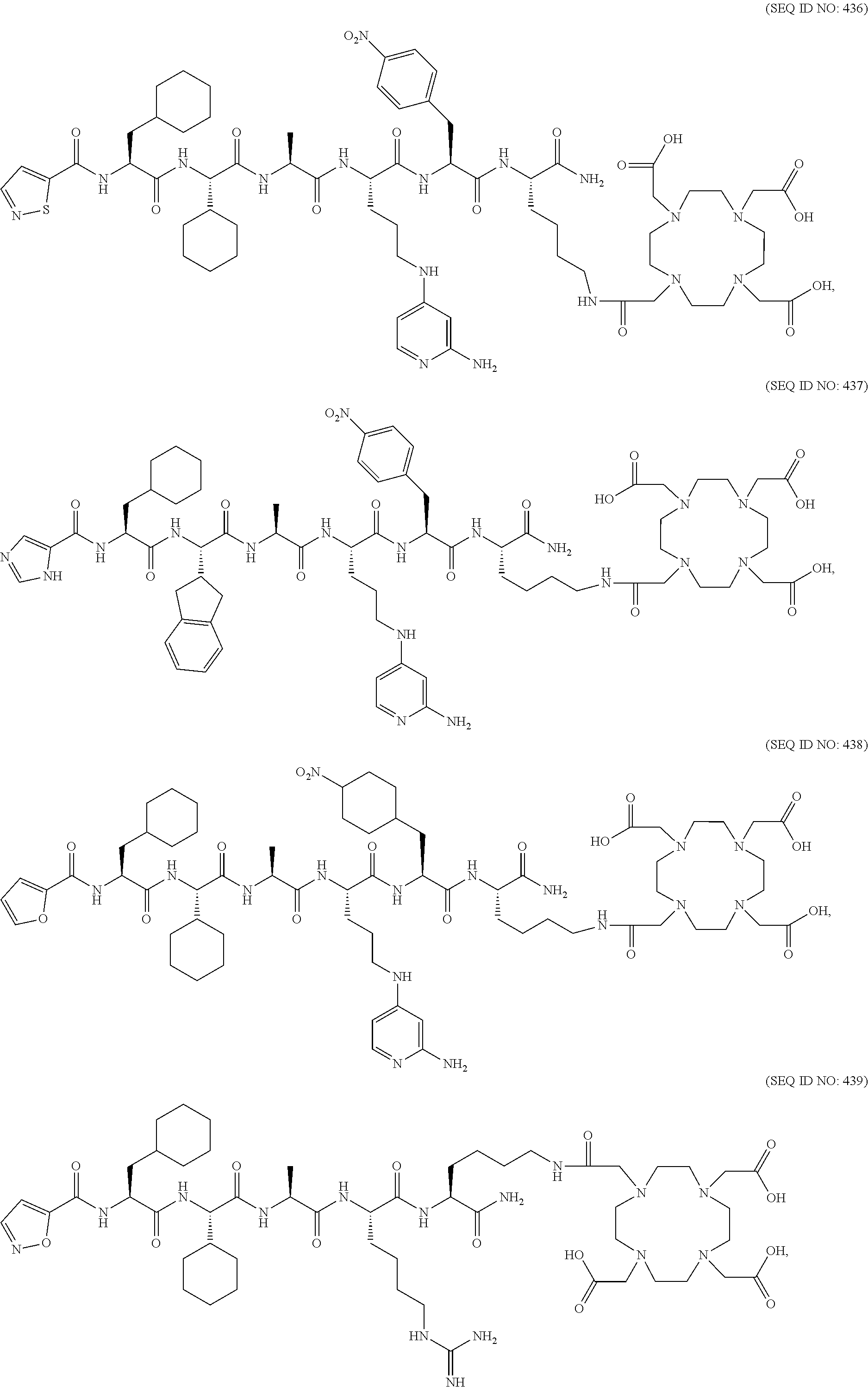 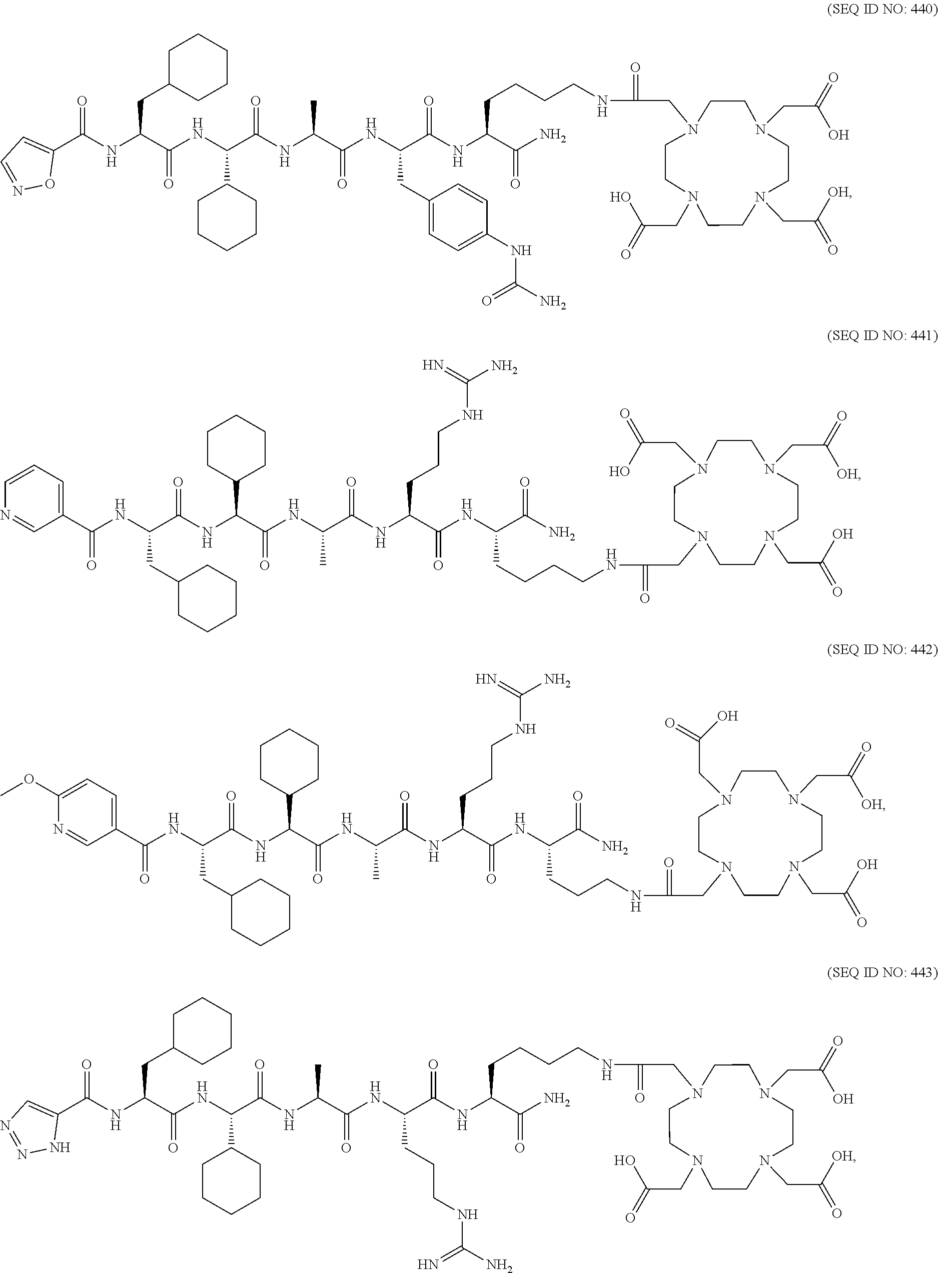 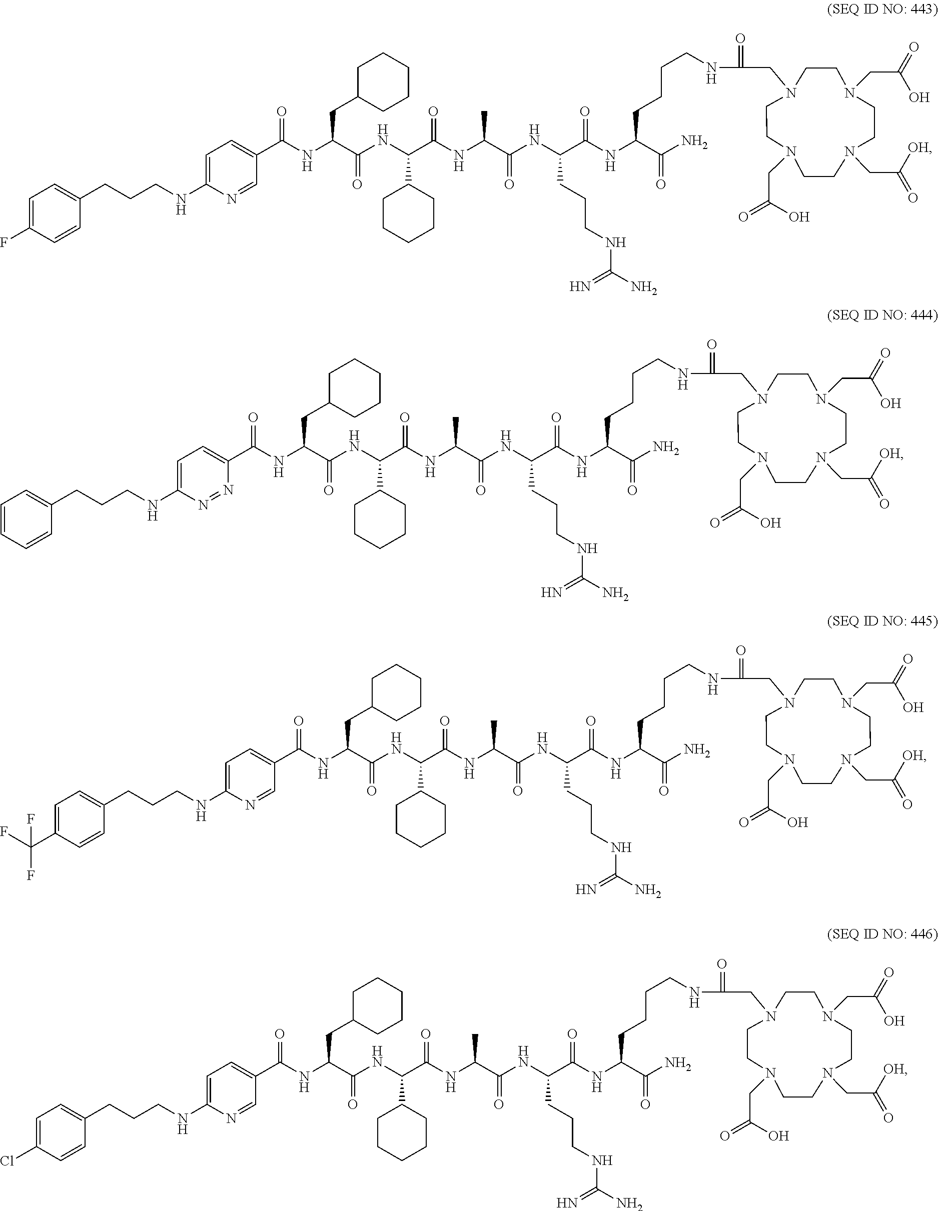 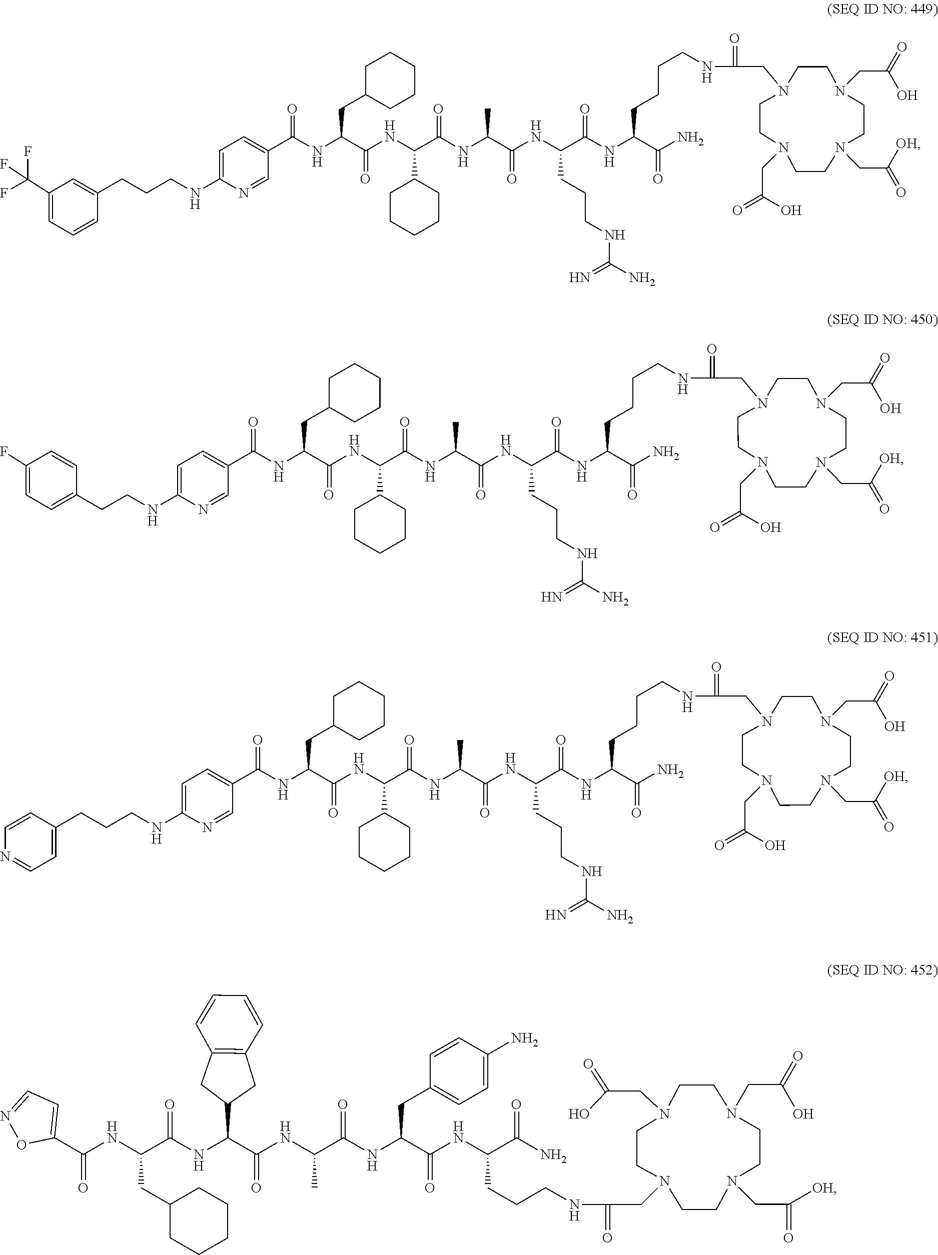 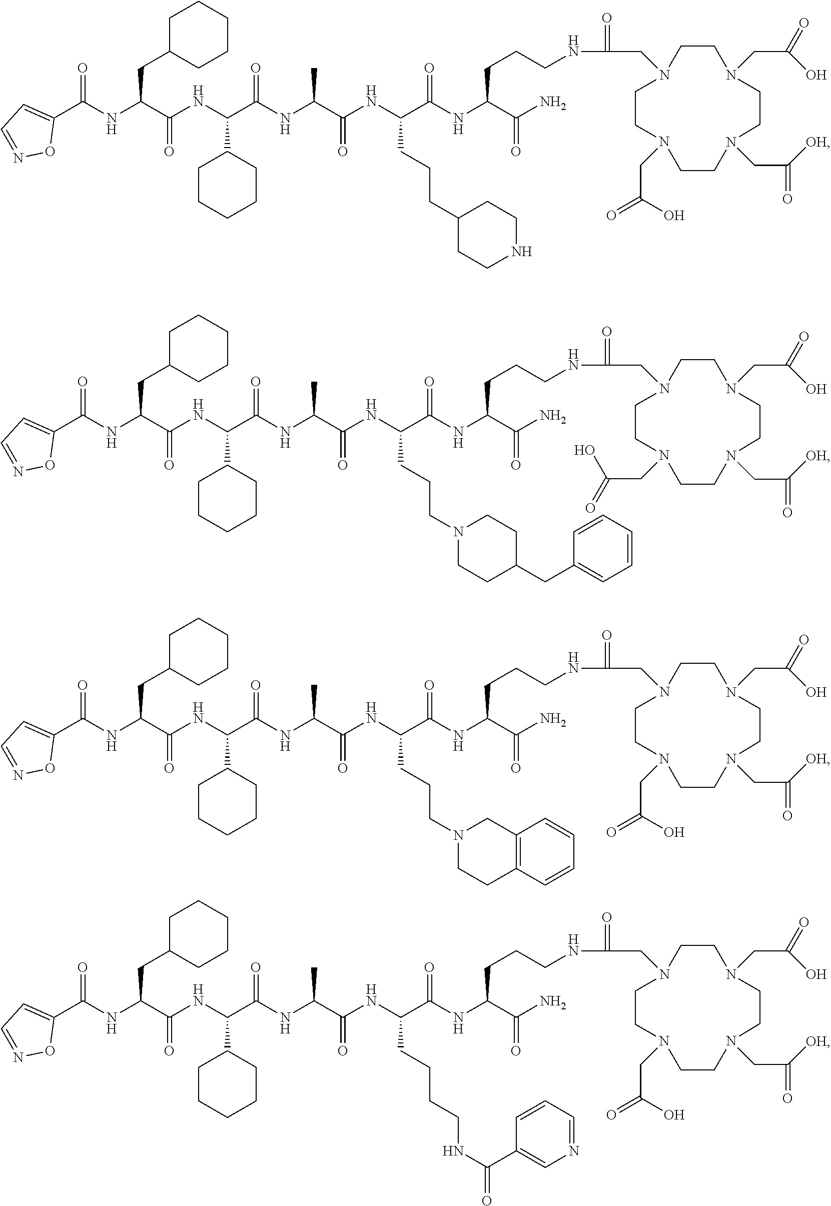 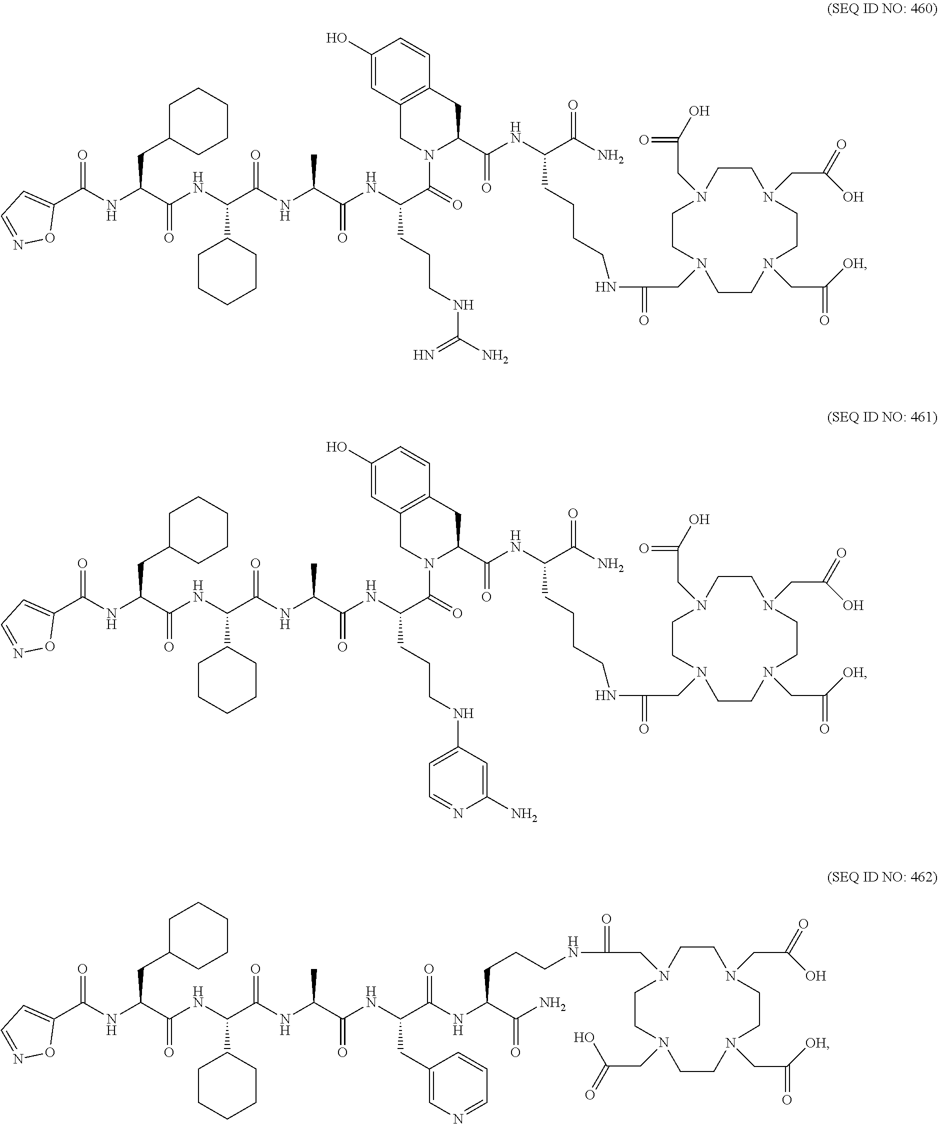 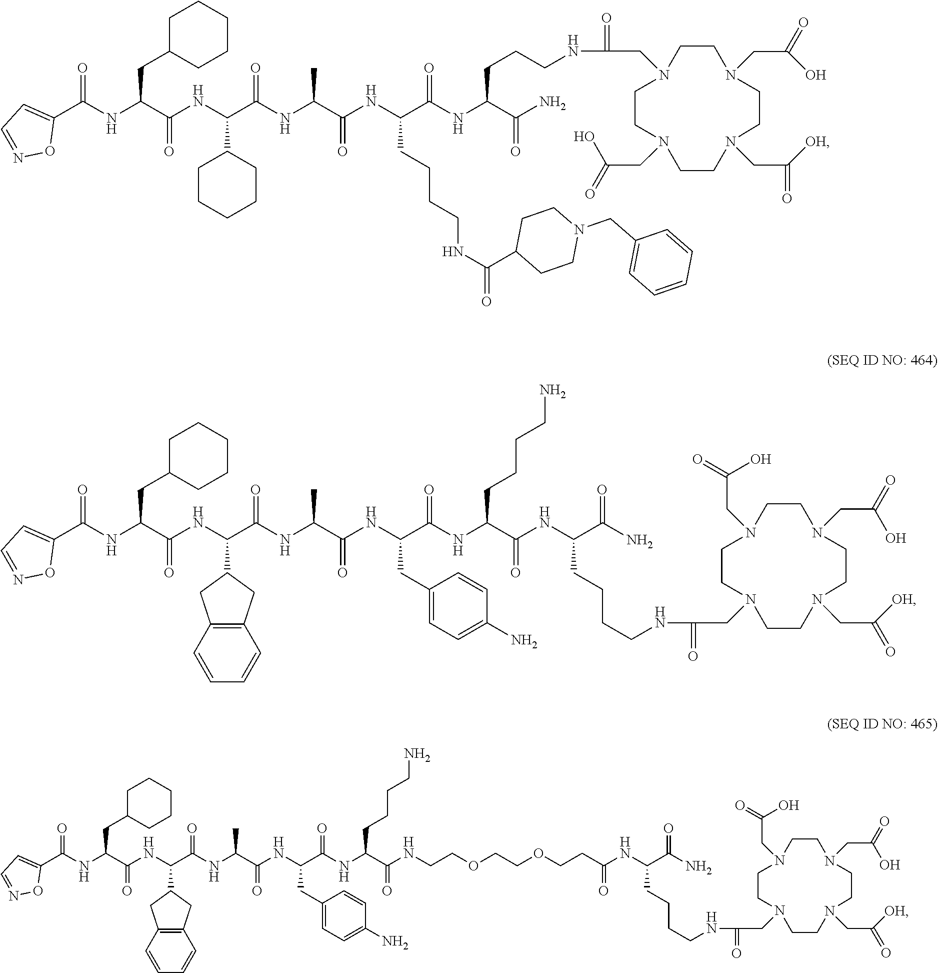 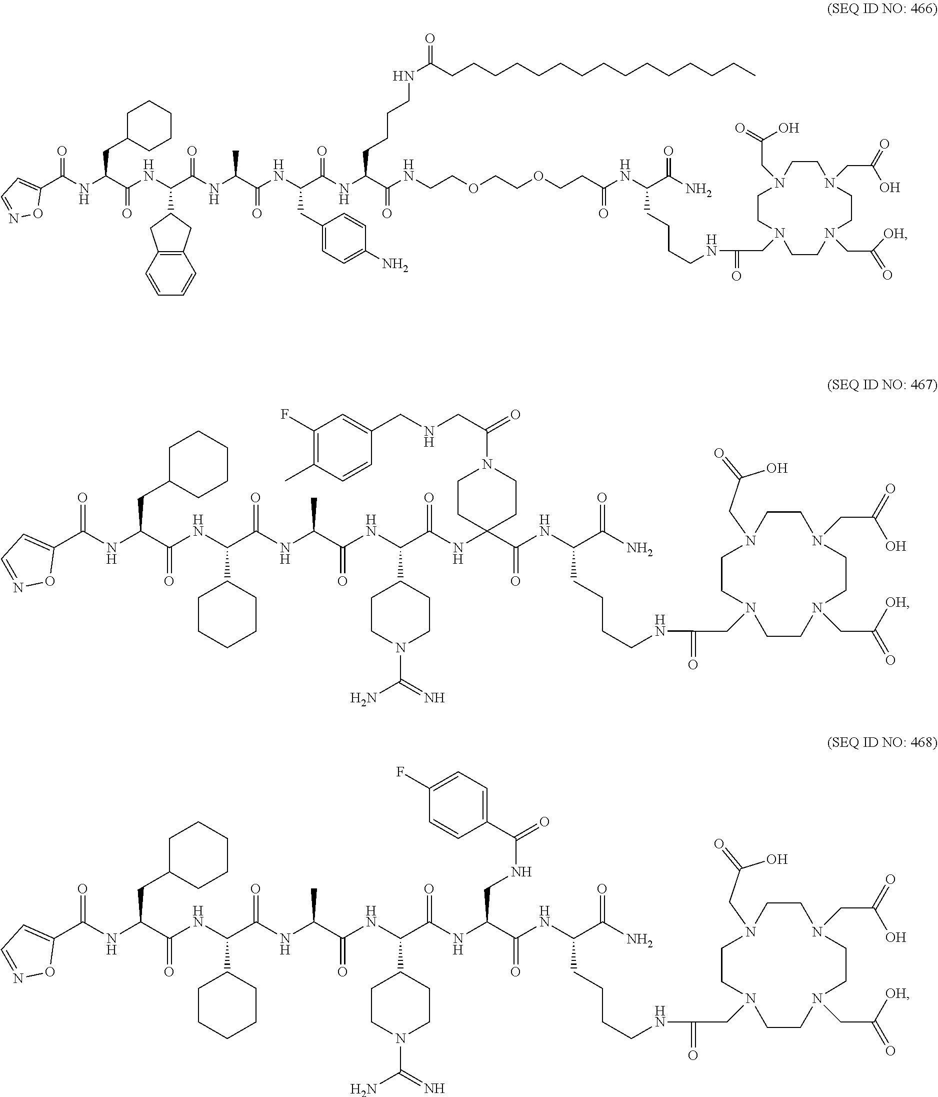 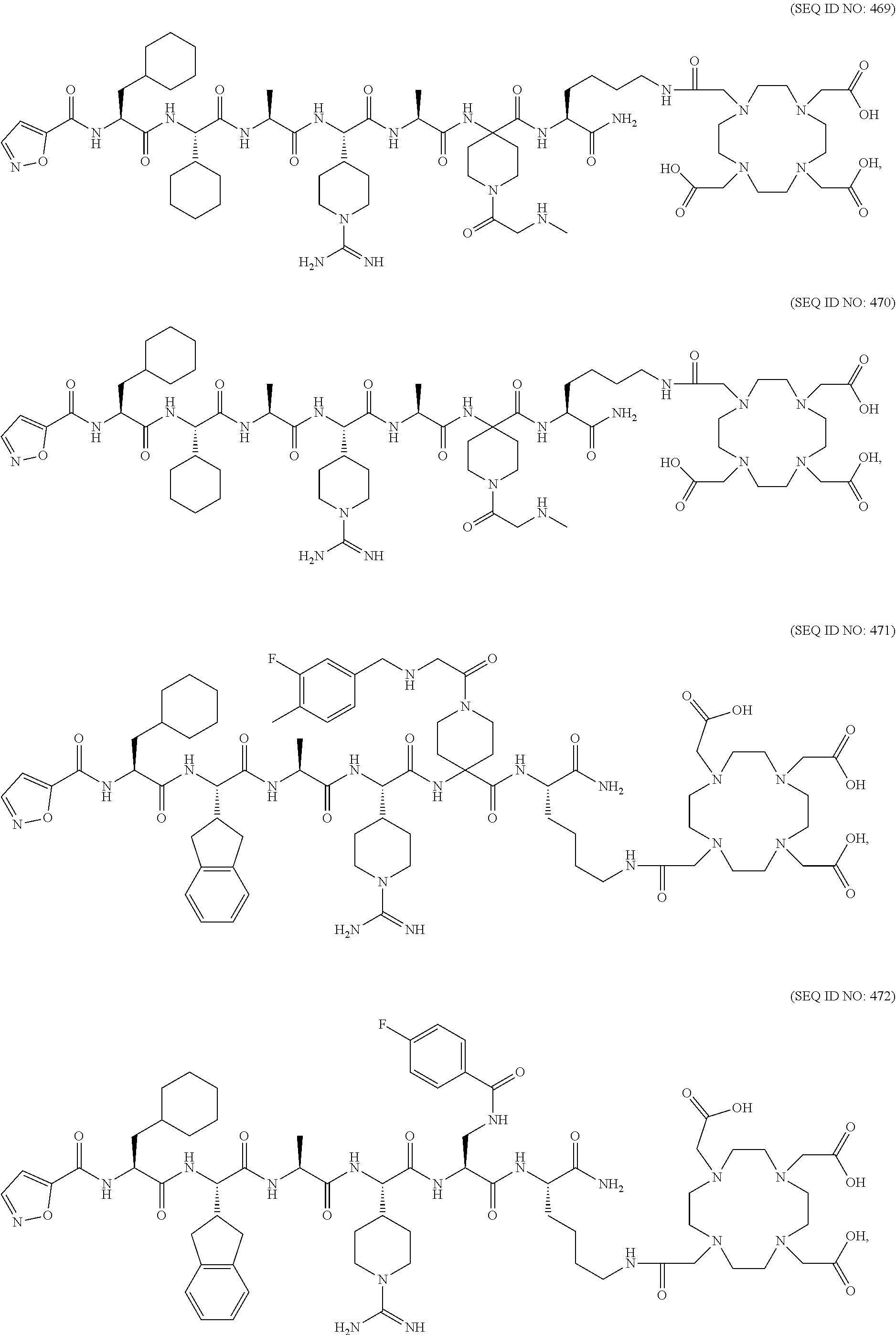 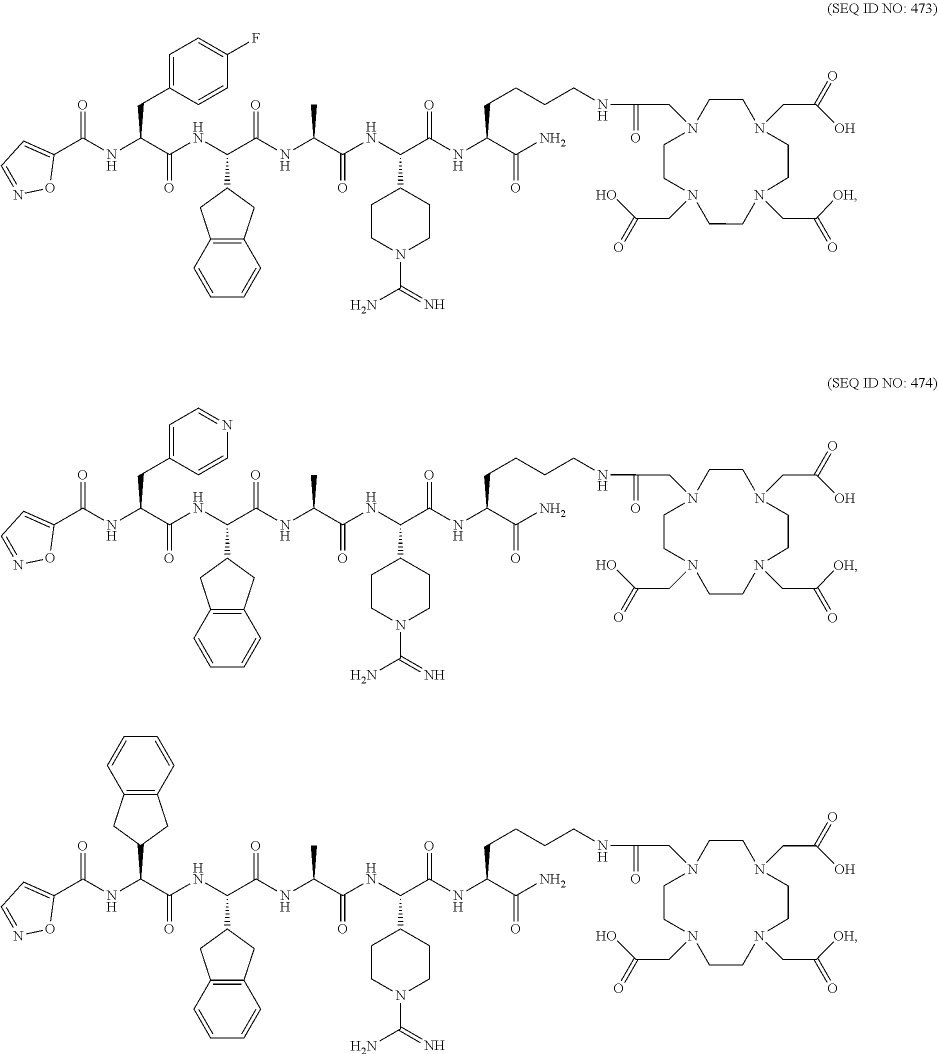 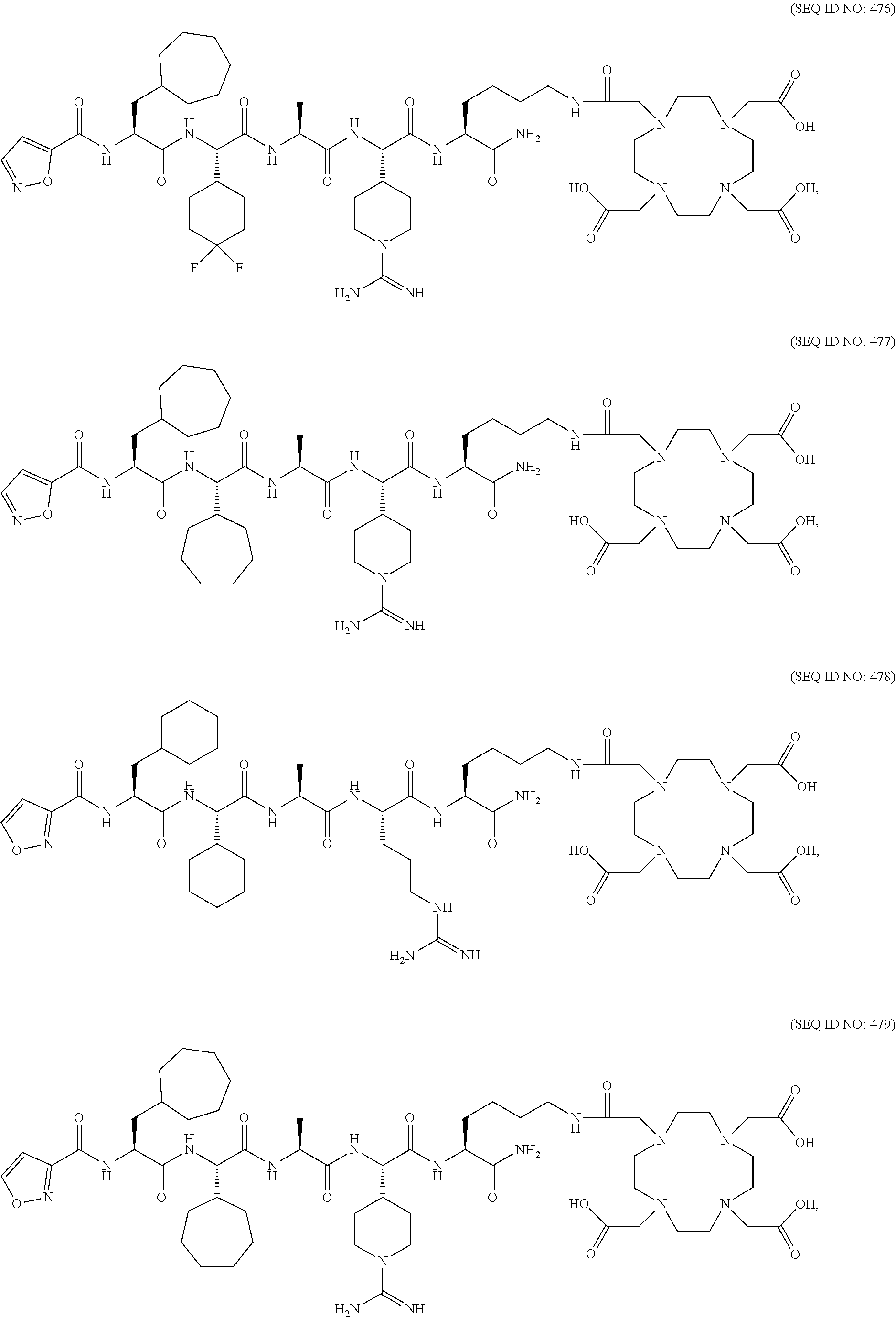 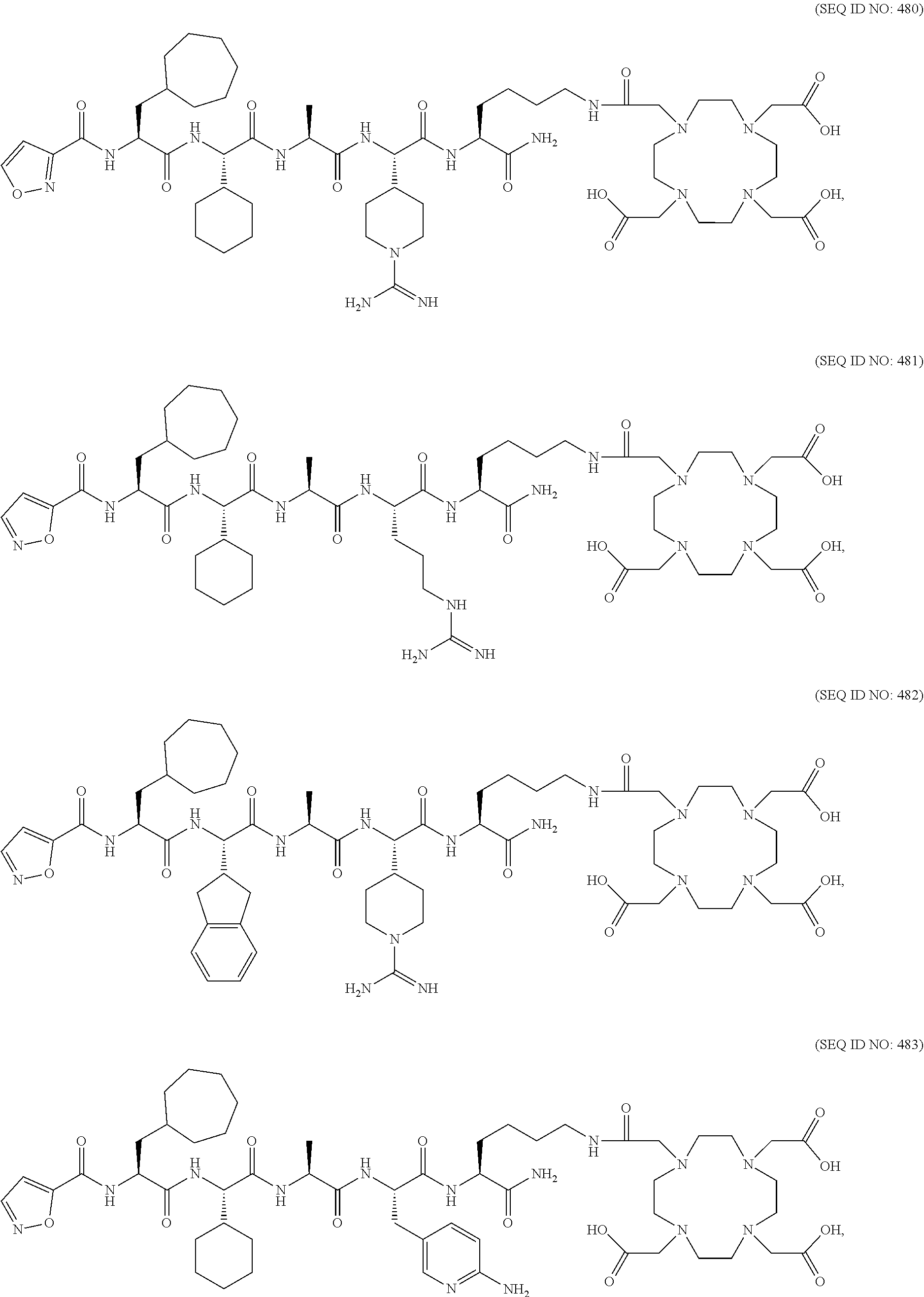 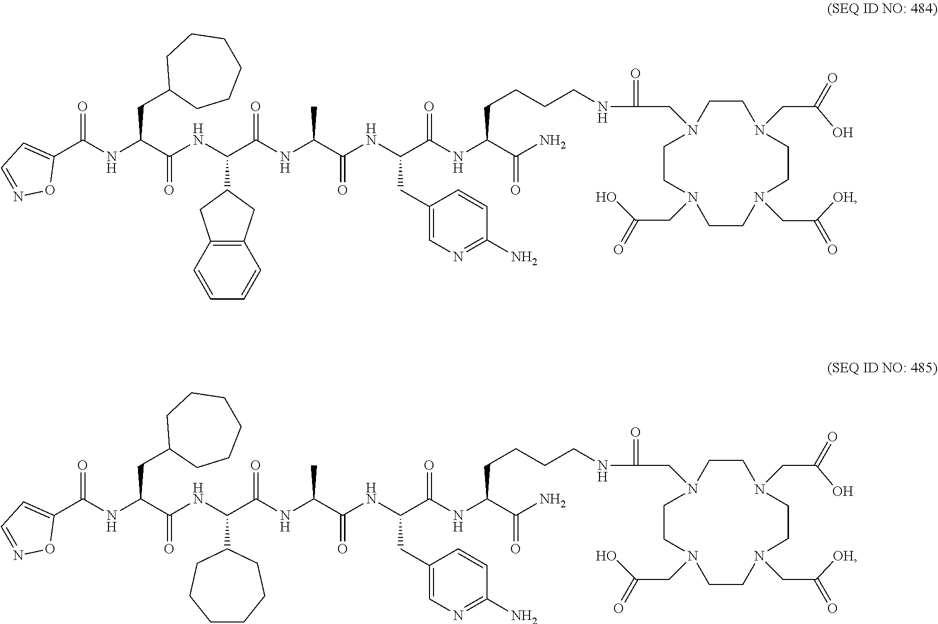 [SMILES: C[C@H](NC(=O)[C@@H](NC(=O)[C@H](CC1CCCCCC1)NC(=O)c1ccno1)C1Cc2ccccc2C1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(N)=O ; C[C@H](NC(=O)[C@@H](NC(=O)[C@H](CC1CCCCCC1)NC(=O)c1ccno1)C1CCCCCC1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(N)=O] 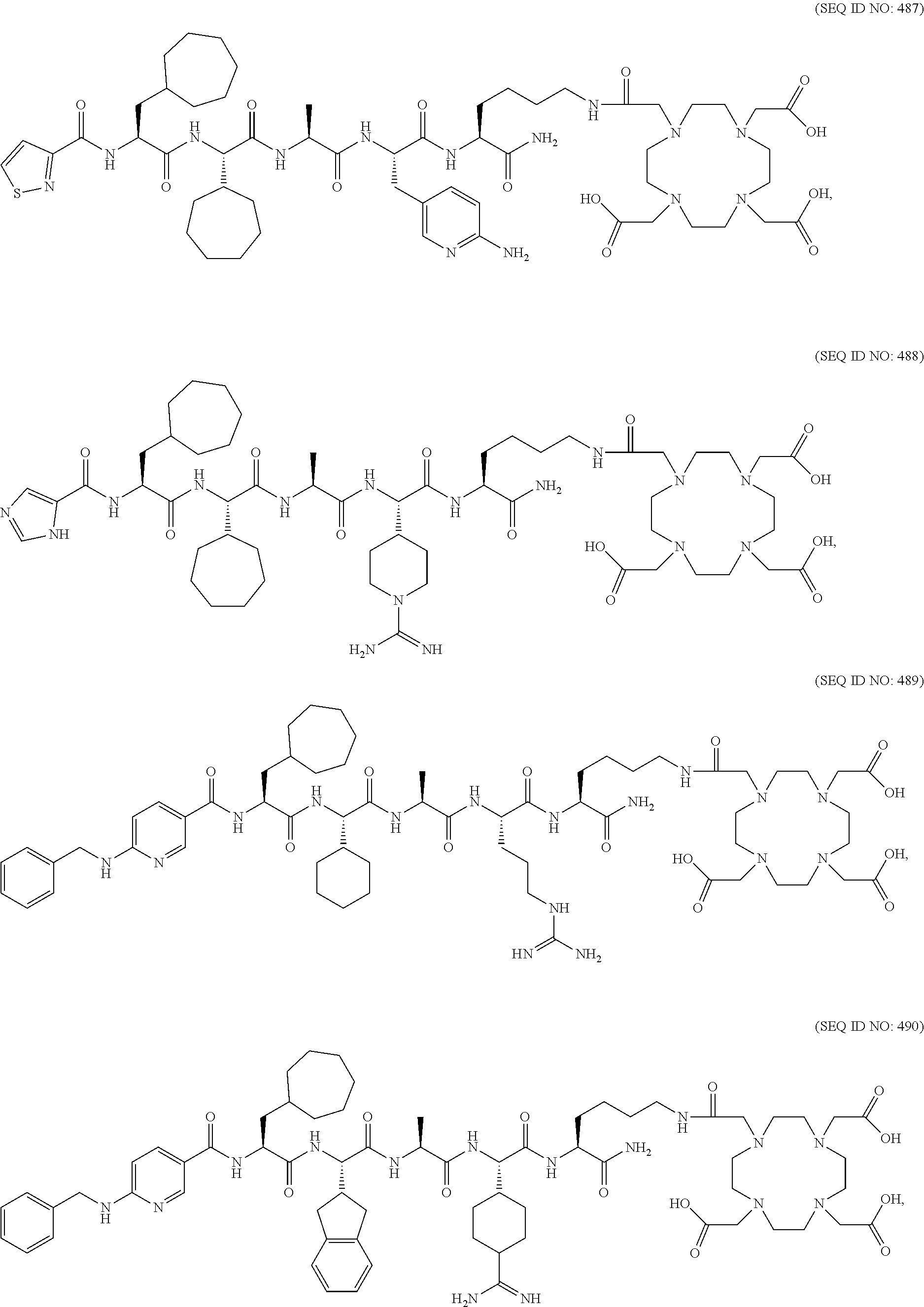 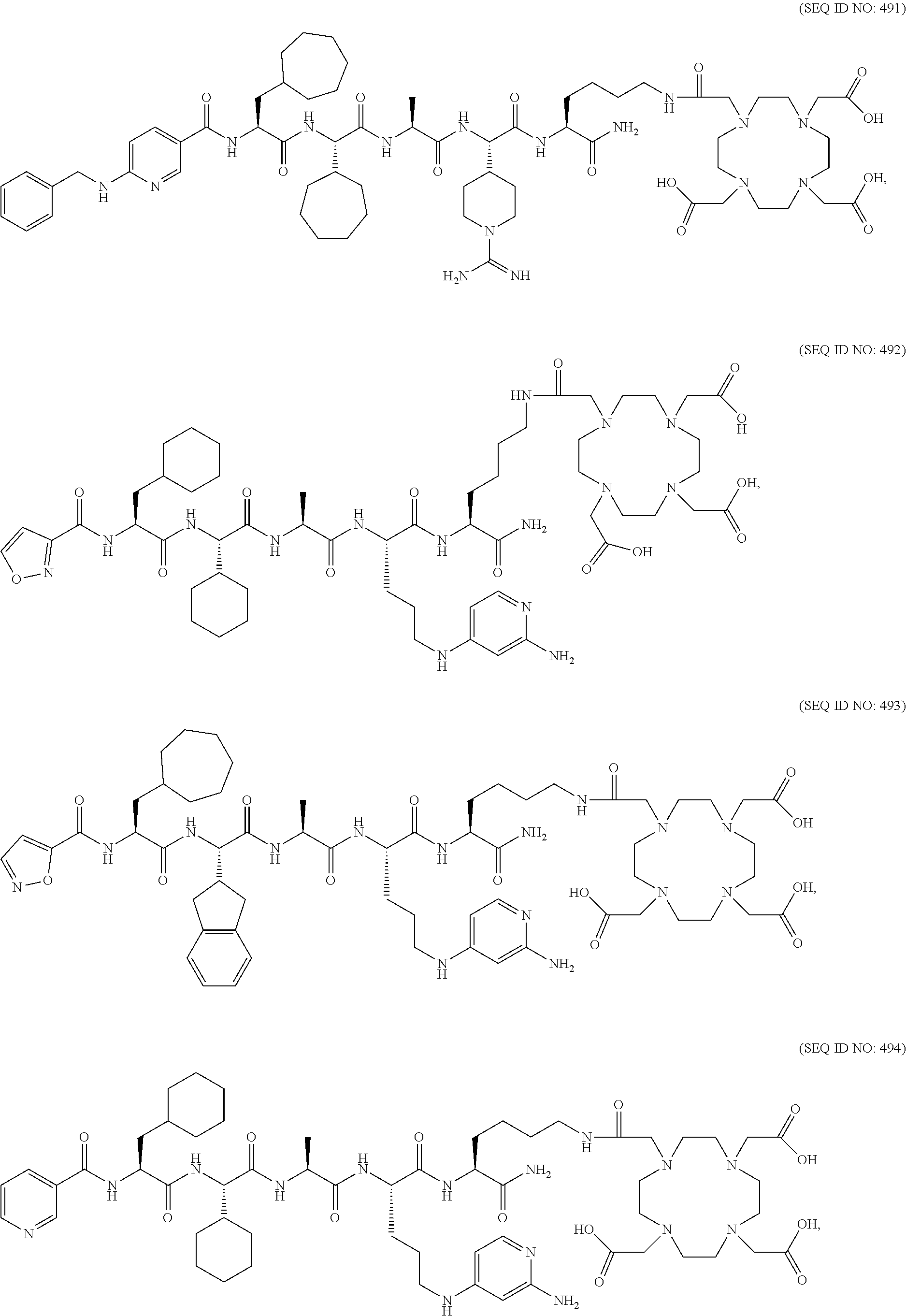 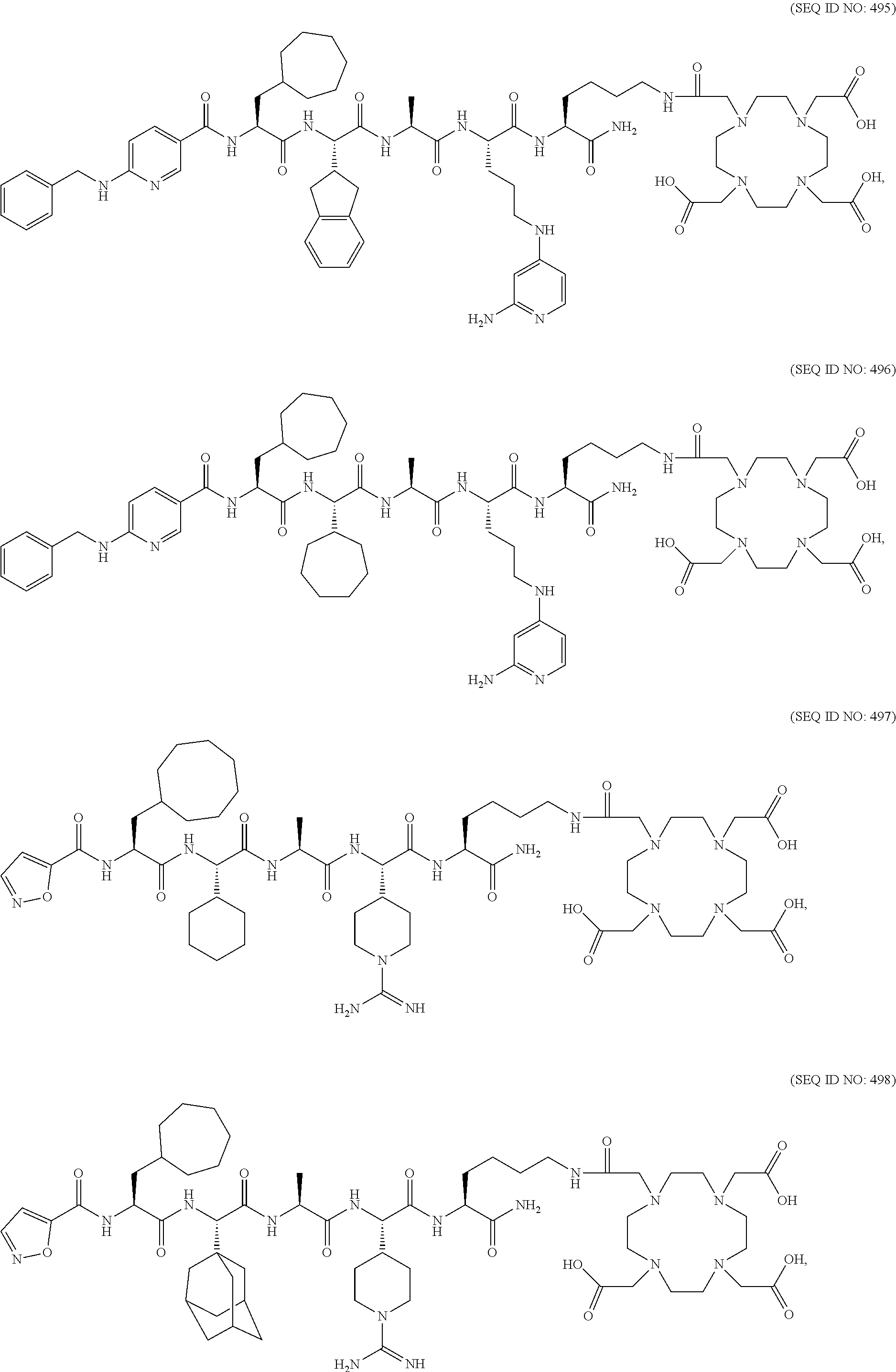 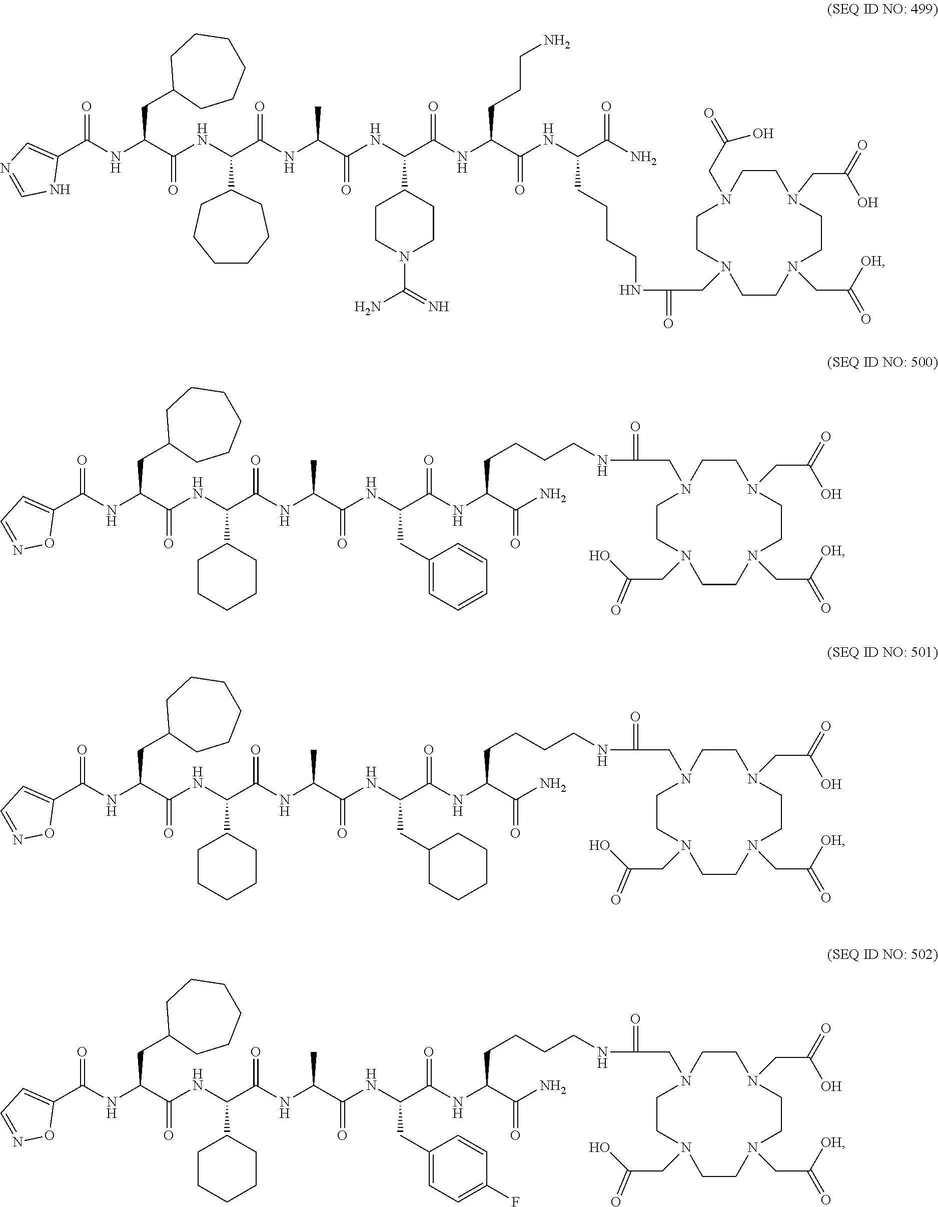 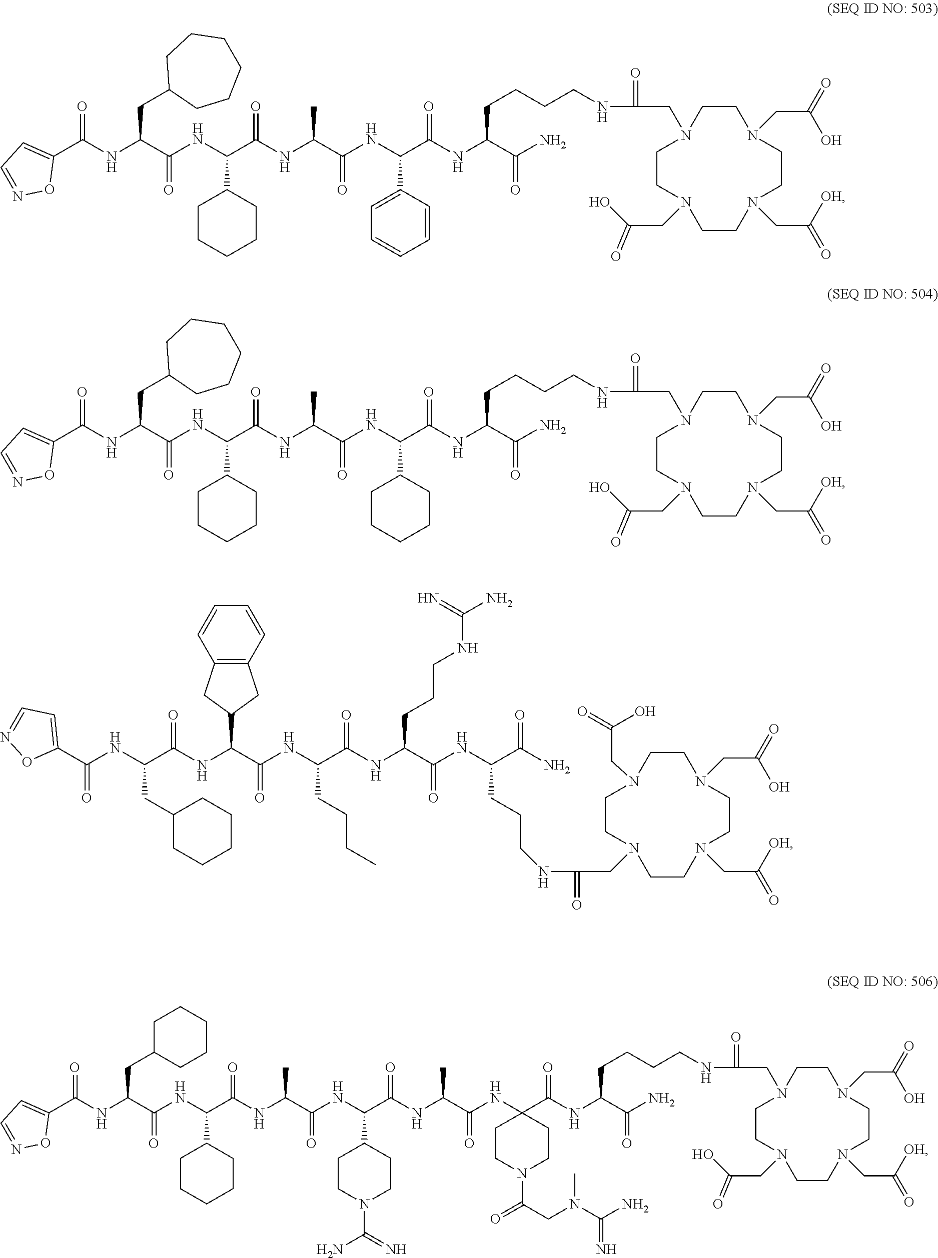 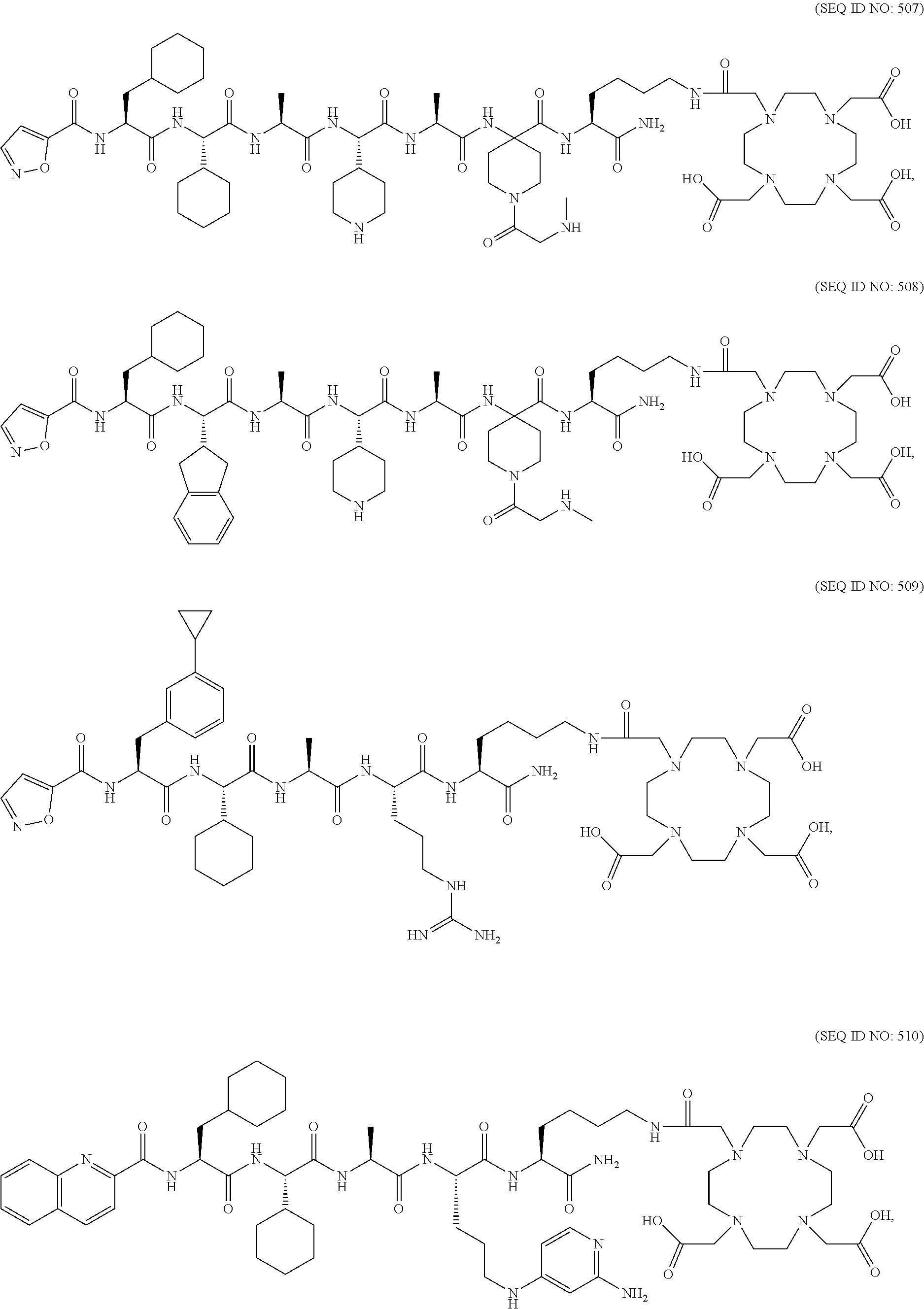 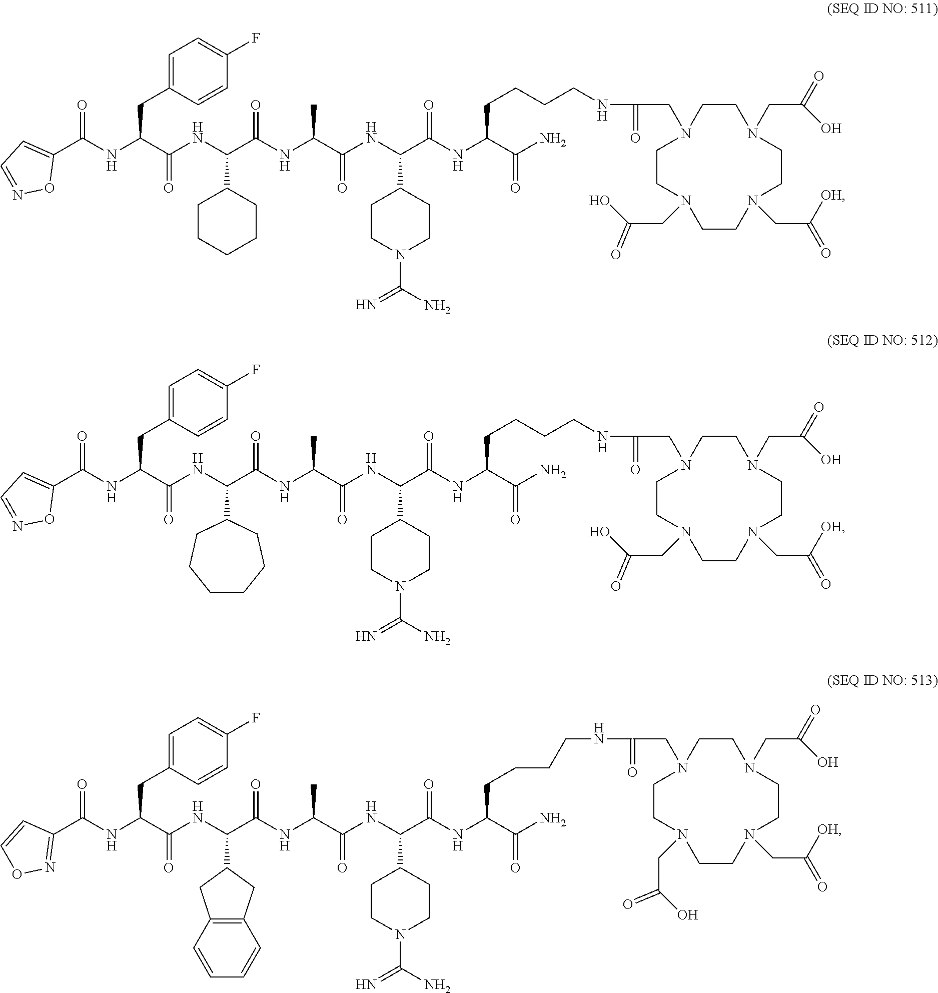 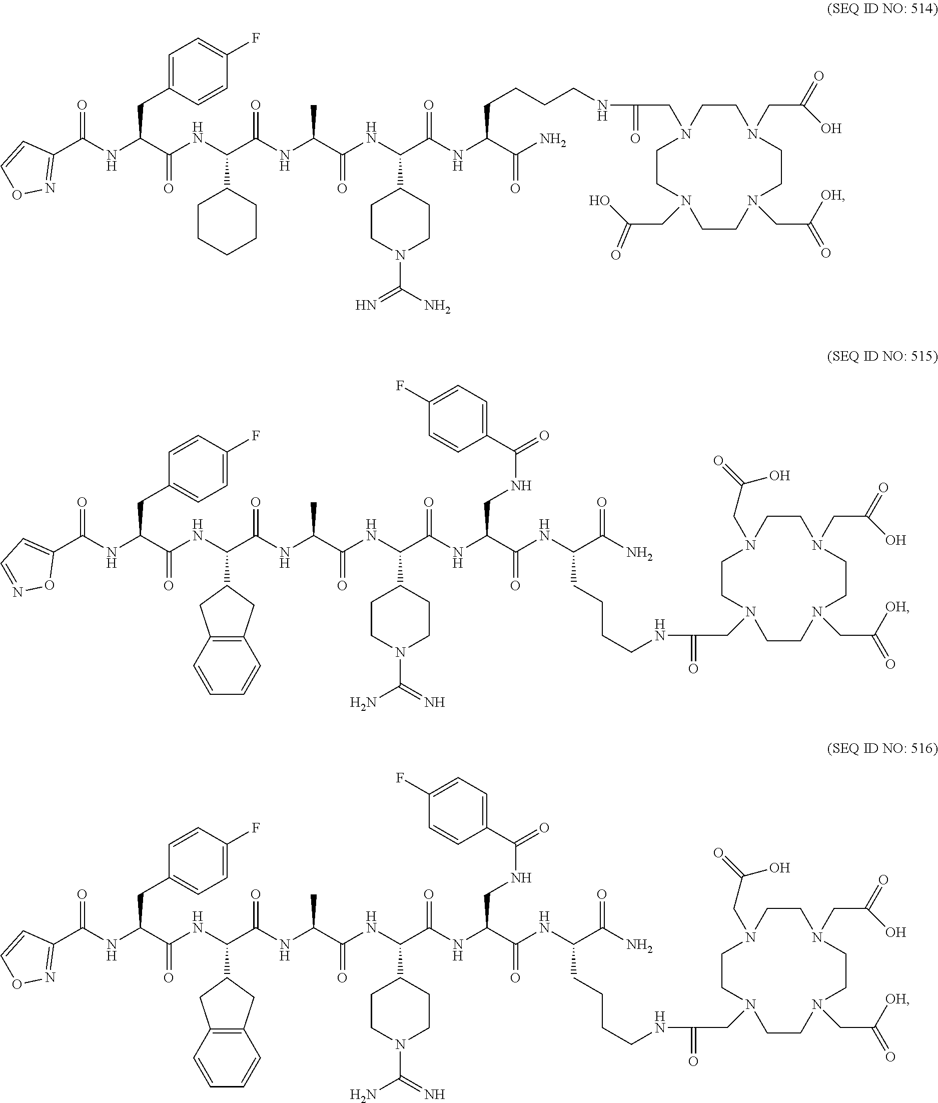 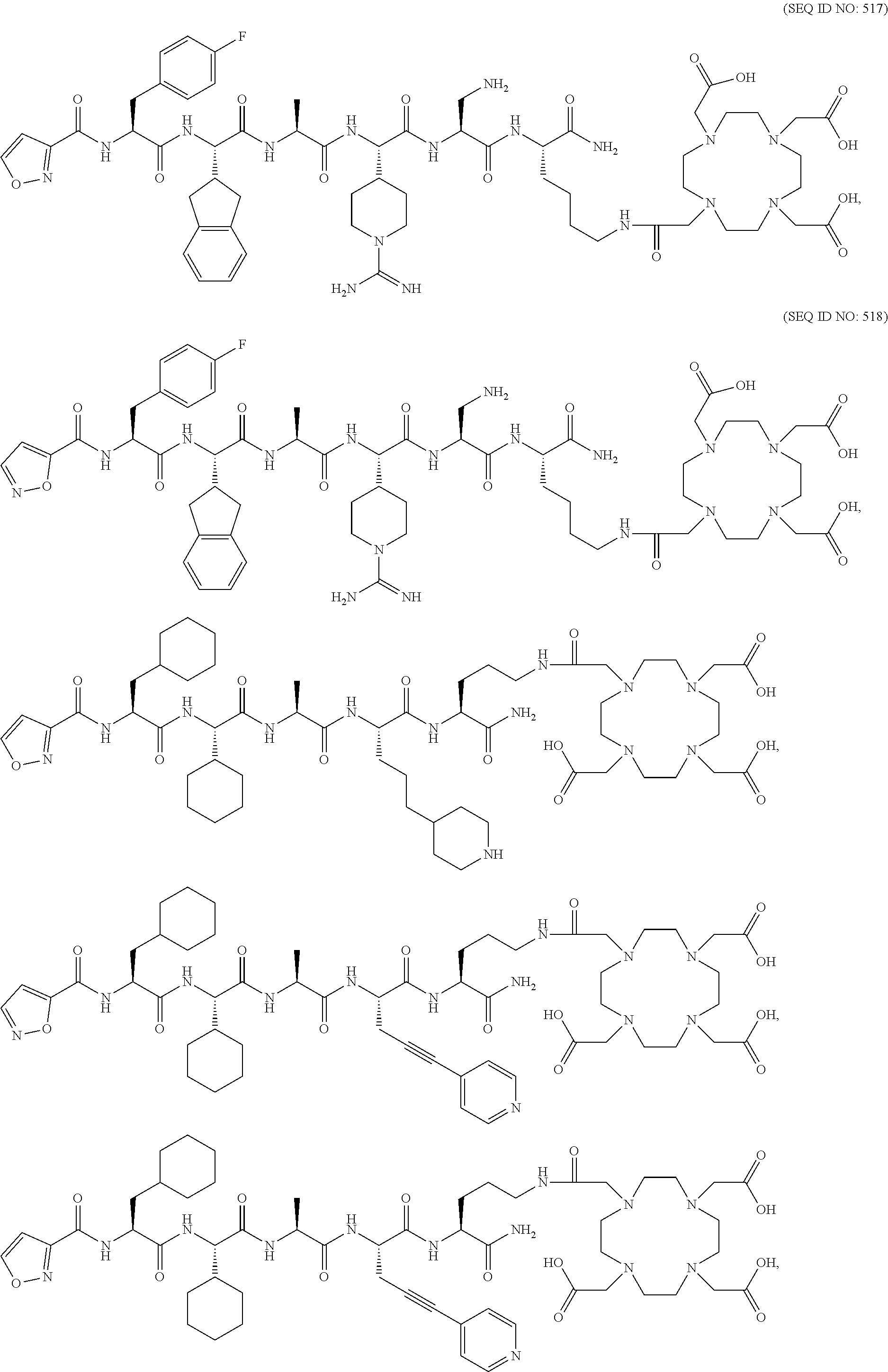 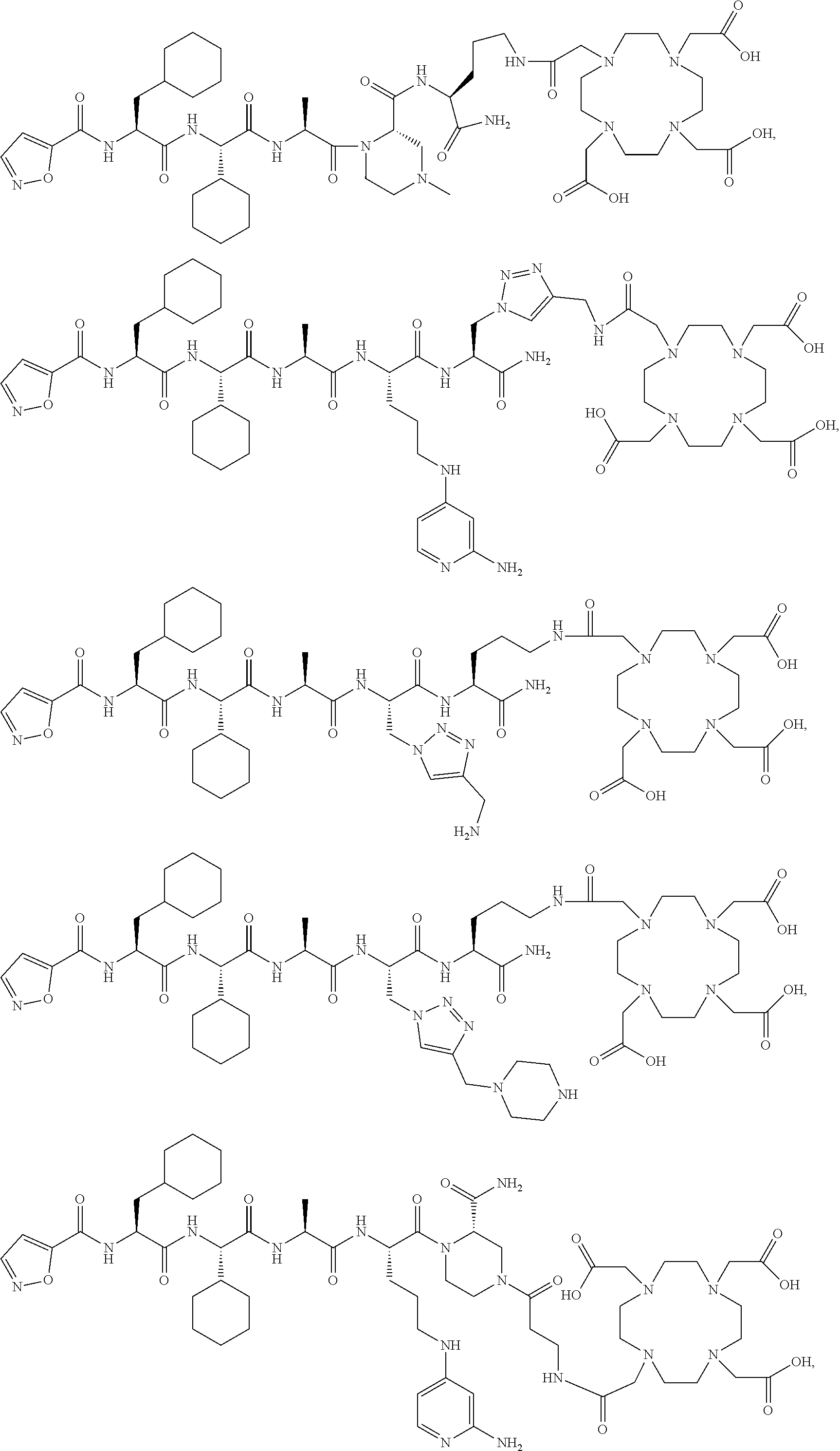 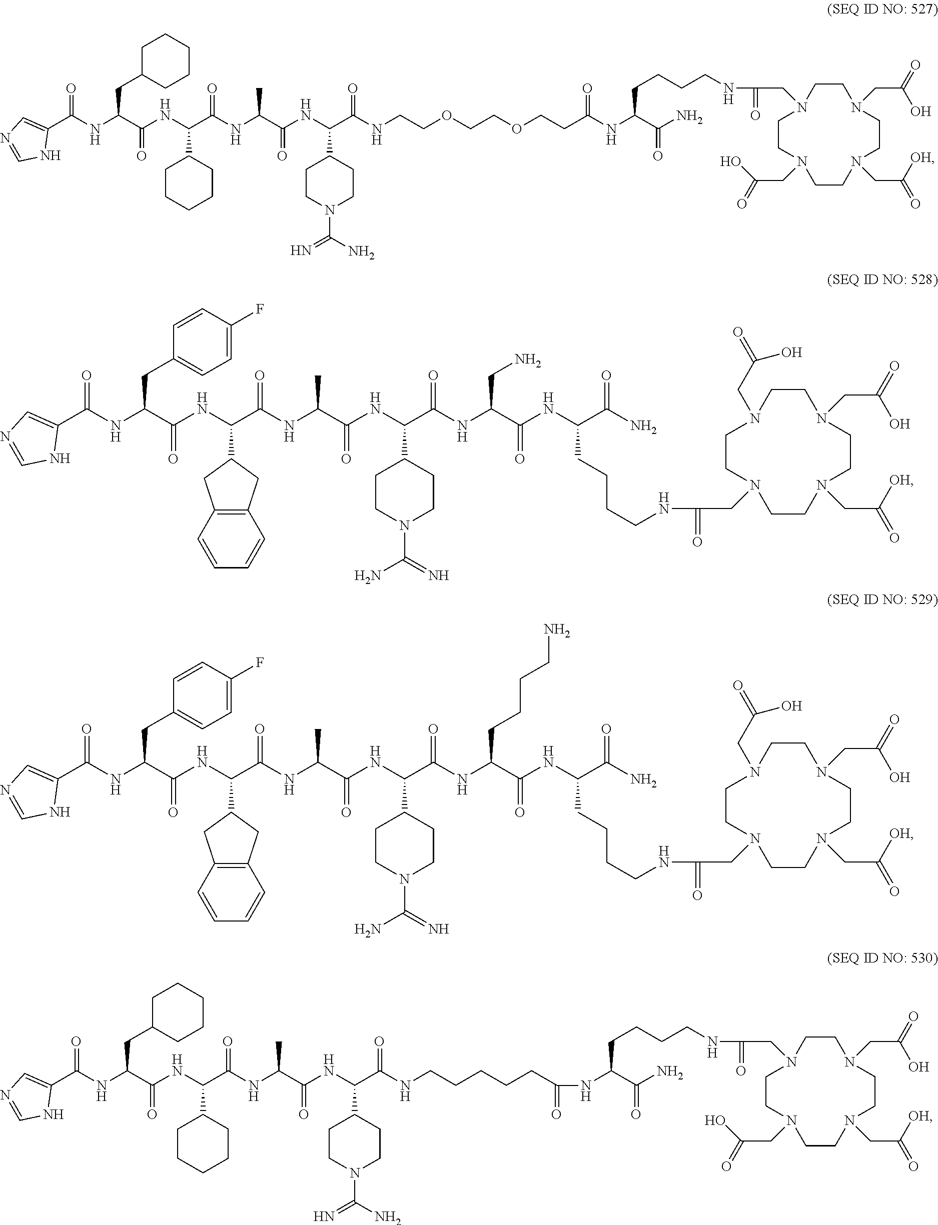 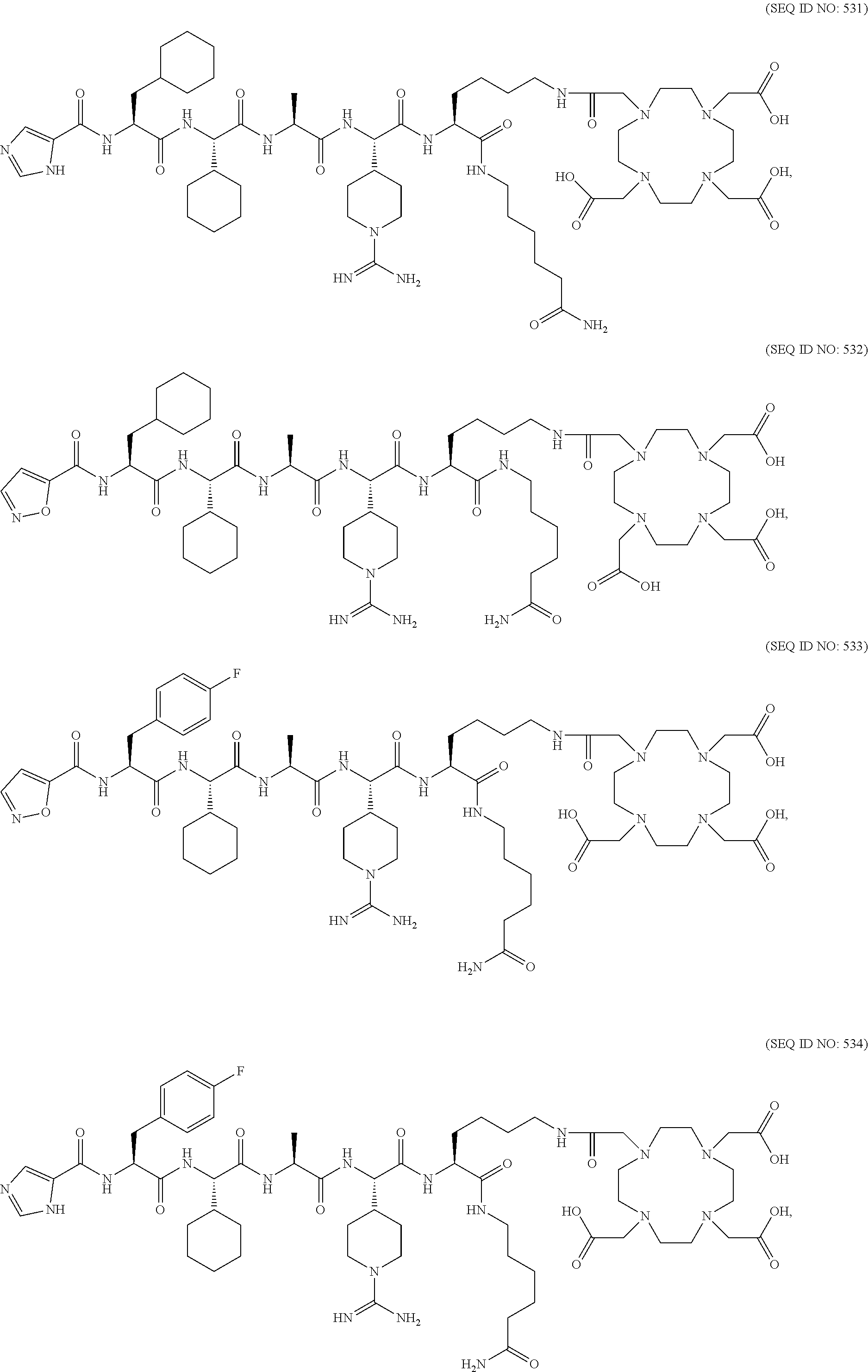 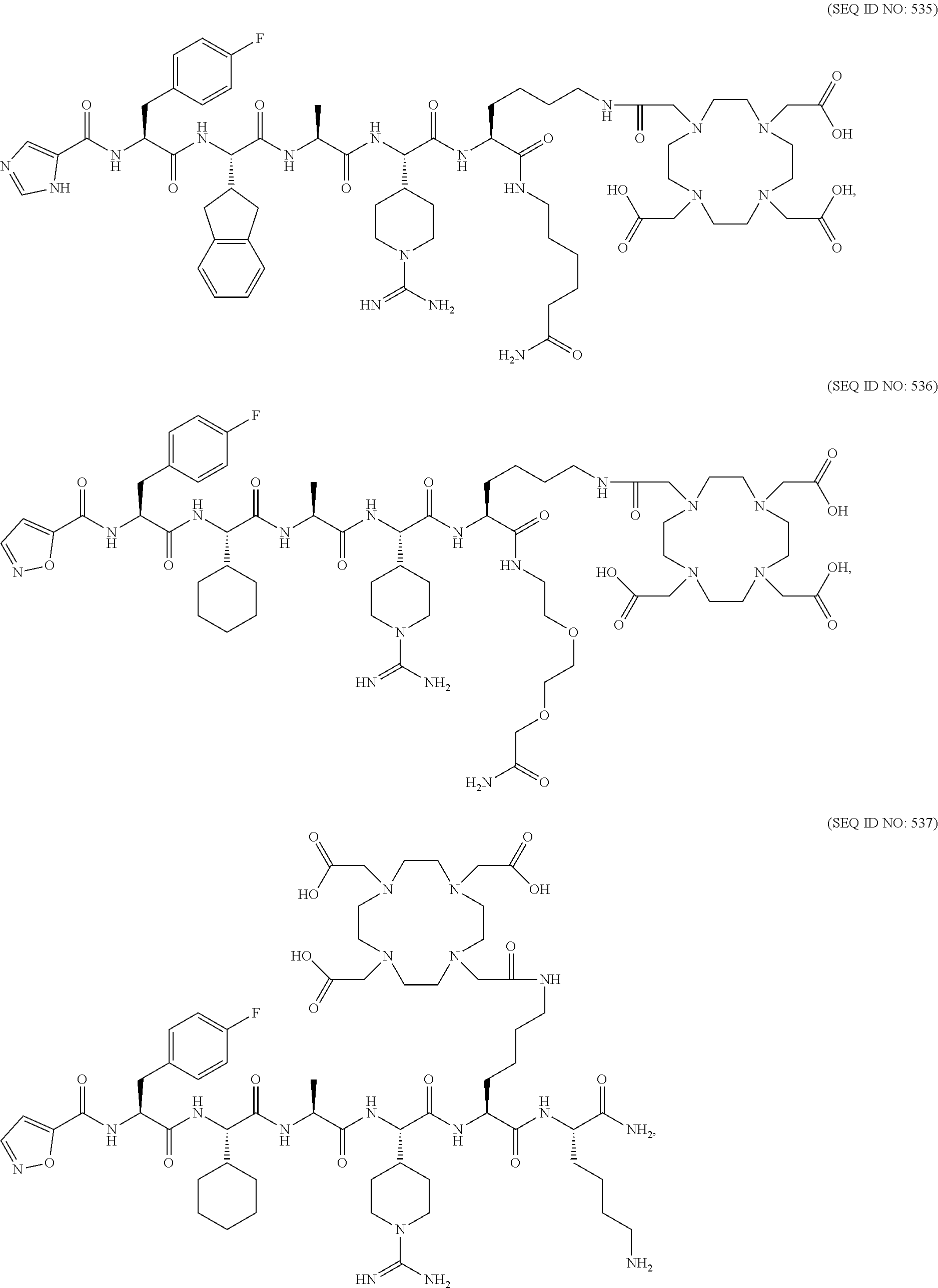 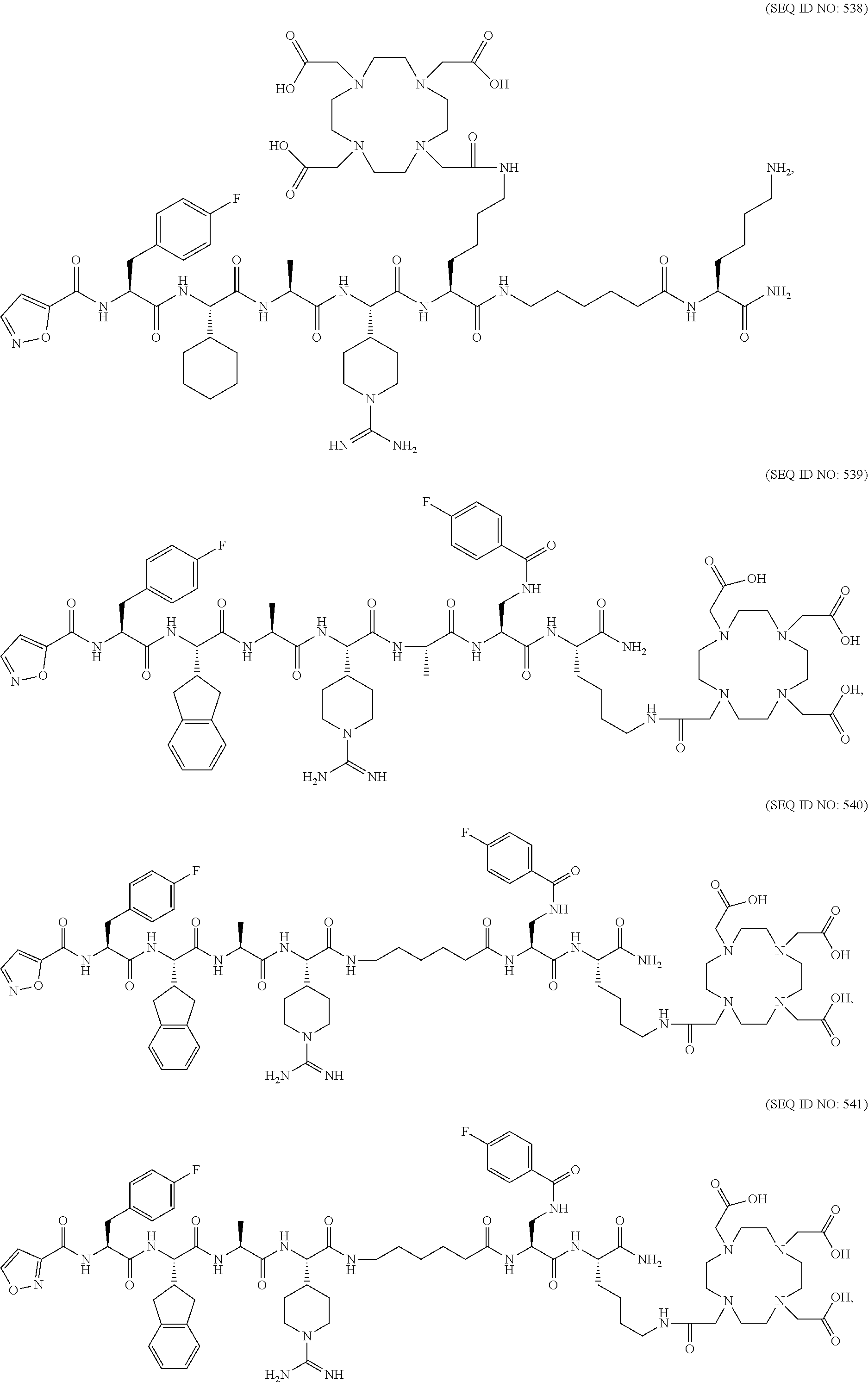 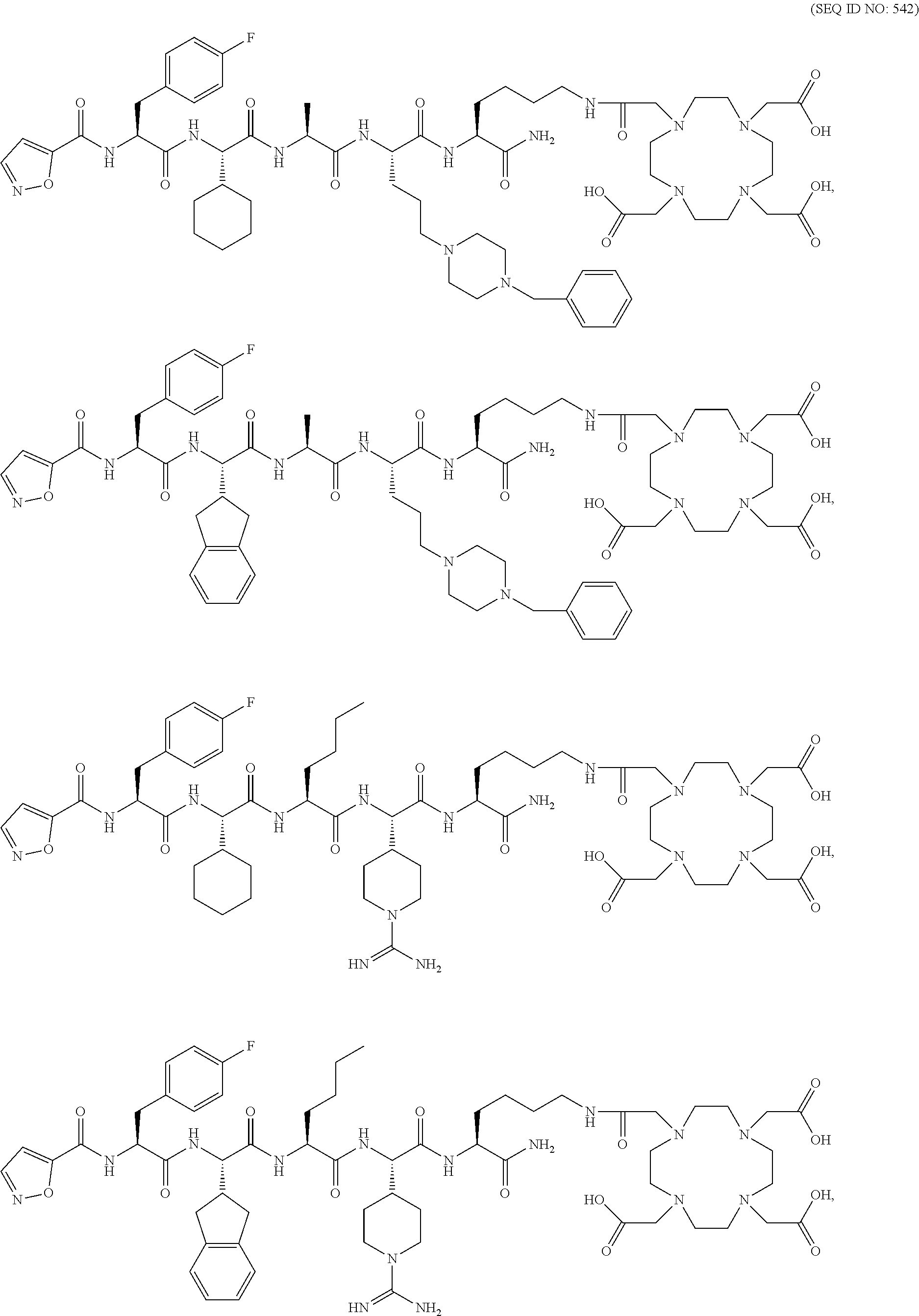 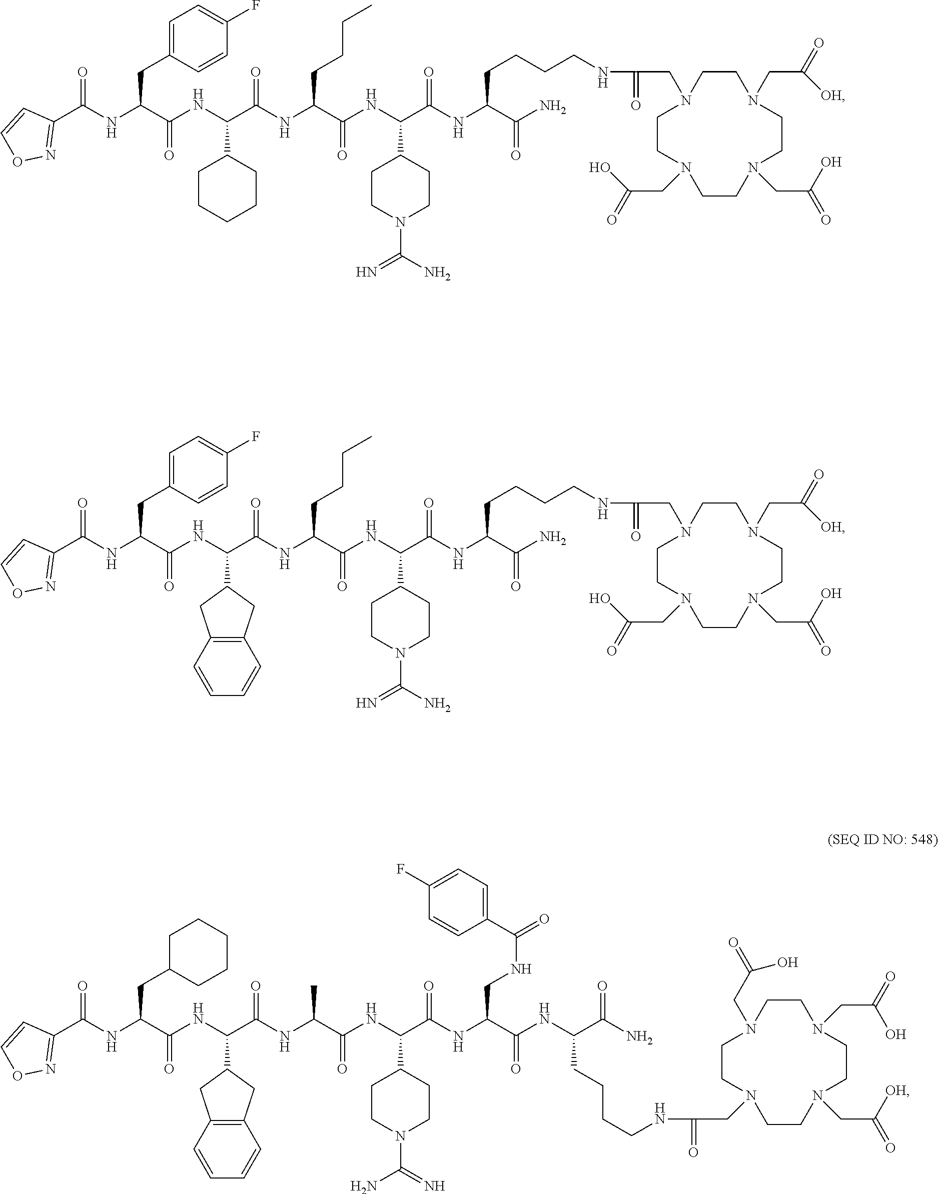 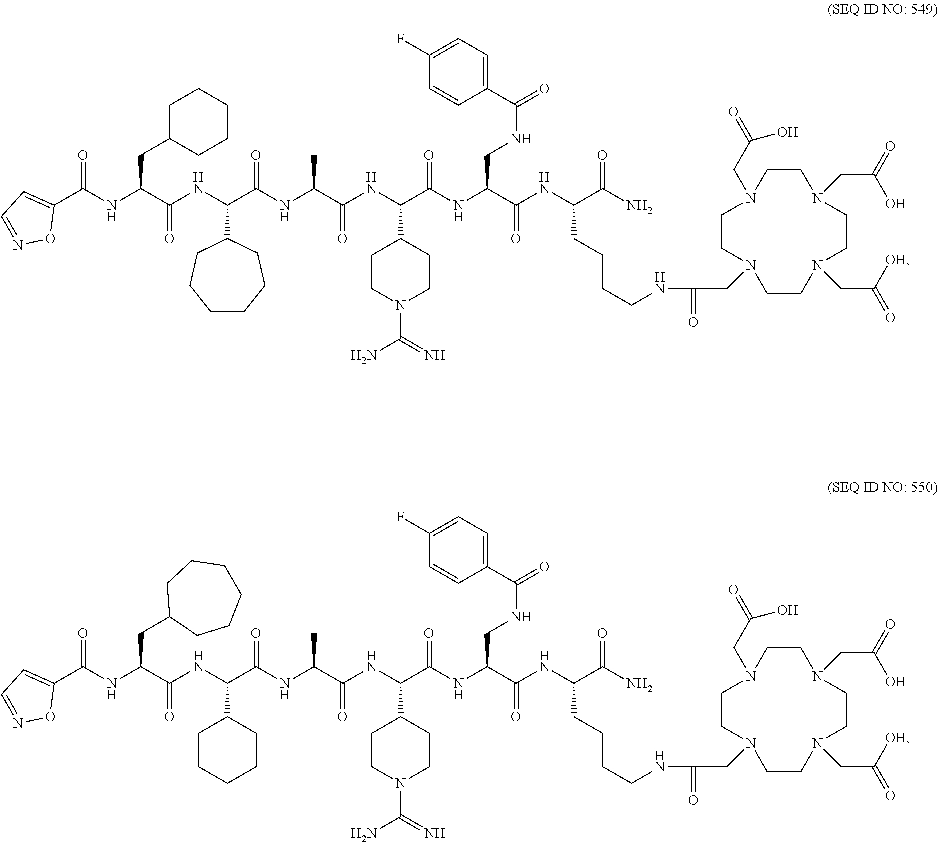 [SMILES: C[C@H](NC(=O)[C@@H](NC(=O)[C@H](CC1CCCCC1)NC(=O)c1ccno1)C1CCCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](CC1CCCCCC1)NC(=O)c1ccno1)C1CCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1] 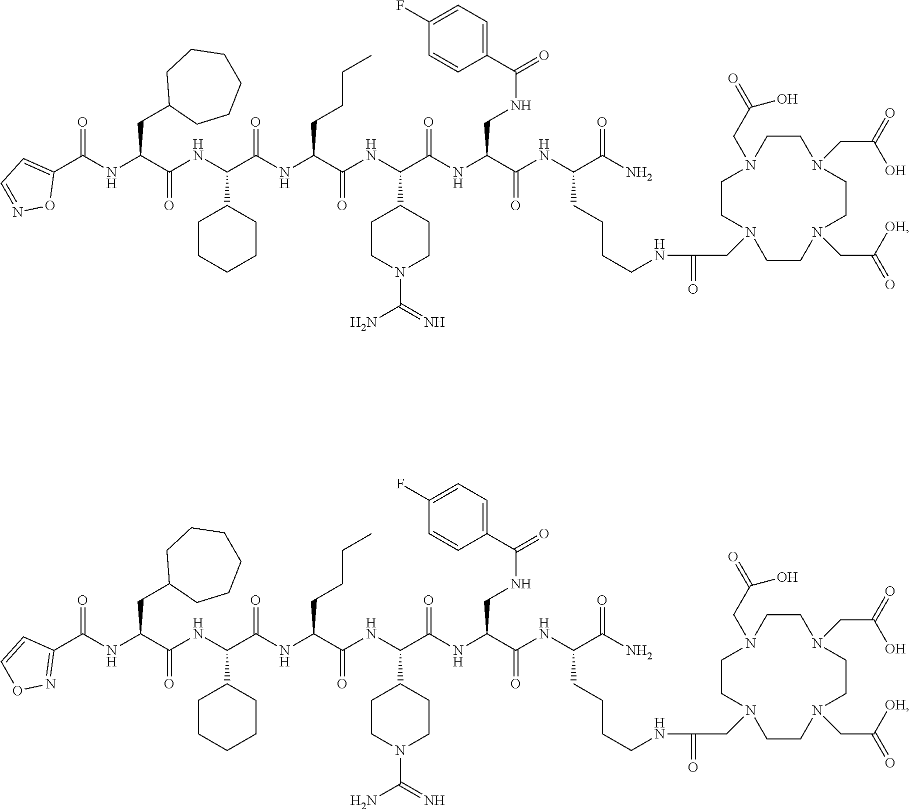 [SMILES: CCCC[C@H](NC(=O)[C@@H](NC(=O)[C@H](CC1CCCCCC1)NC(=O)c1ccno1)C1CCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; CCCC[C@H](NC(=O)[C@@H](NC(=O)[C@H](CC1CCCCCC1)NC(=O)c1ccon1)C1CCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1] 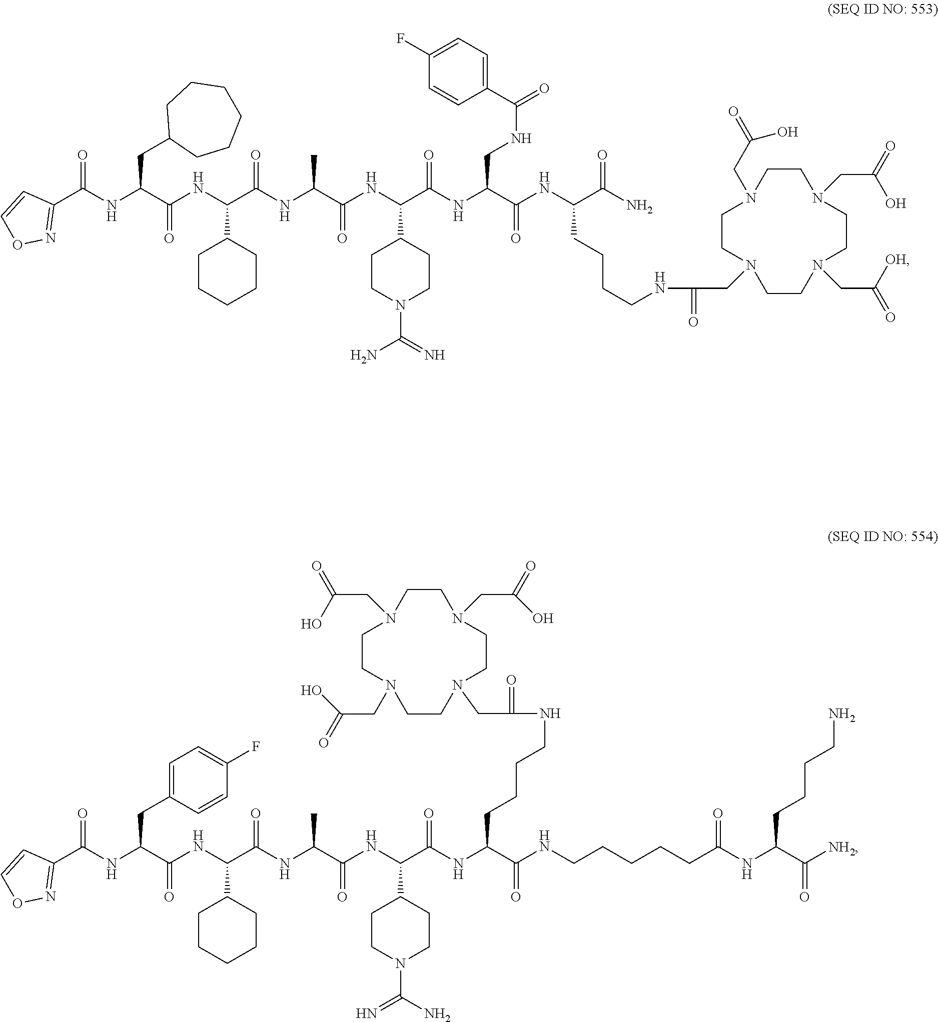 [SMILES: C[C@H](NC(=O)[C@@H](NC(=O)[C@H](CC1CCCCCC1)NC(=O)c1ccon1)C1CCCCC1)C(=O)N[C@H](C(=O)N[C@@H](CNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1] 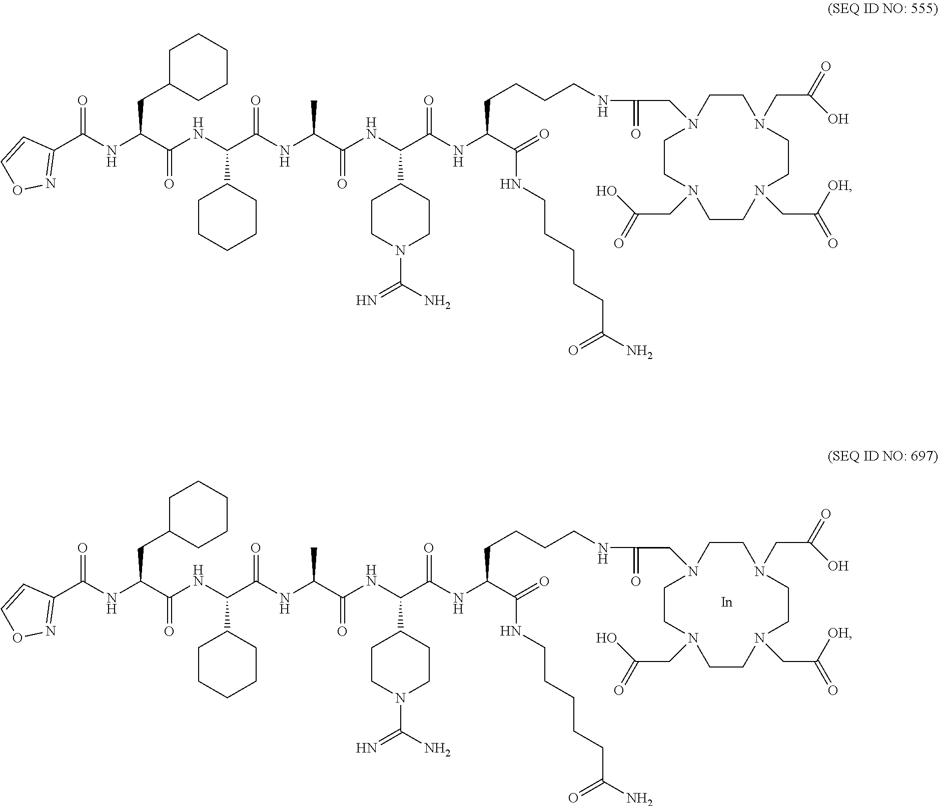 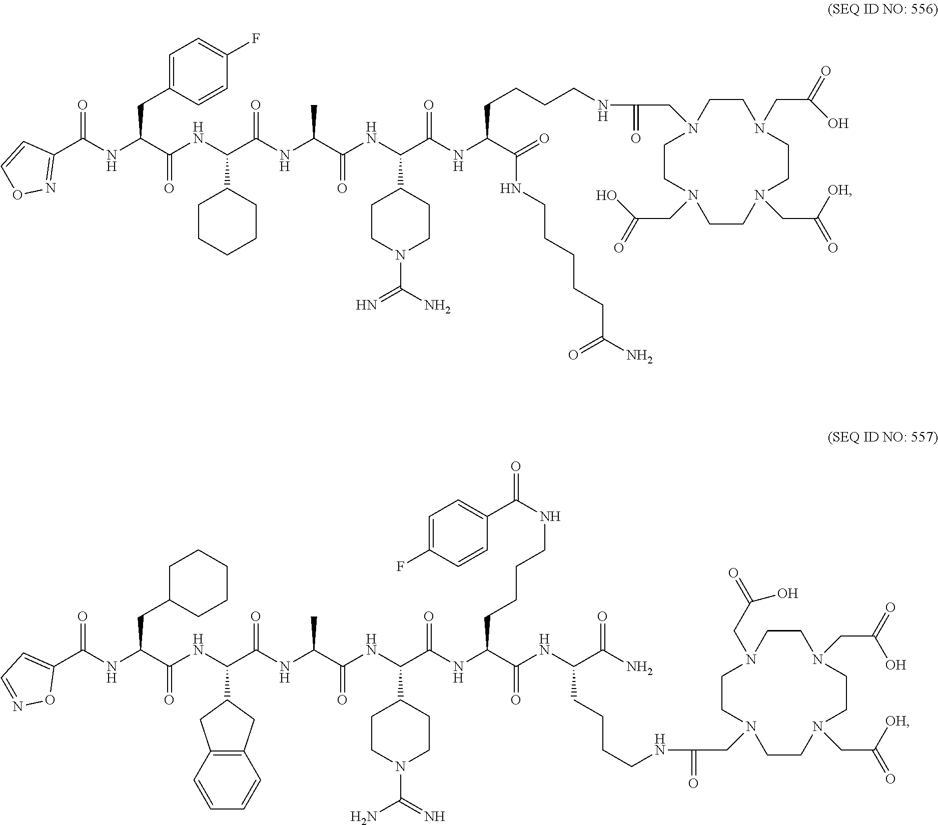 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1 ; C[C@H](NC(=O)[C@@H](NC(=O)[C@H](CC1CCCCC1)NC(=O)c1ccno1)C1Cc2ccccc2C1)C(=O)N[C@H](C(=O)N[C@@H](CCCCNC(=O)c1ccc(F)cc1)C(=O)N[C@@H](CCCCNC(=O)CN1CCN(CC(=O)O)CCN(CC(=O)O)CCN(CC(=O)O)CC1)C(N)=O)C1CCN(C(=N)N)CC1] 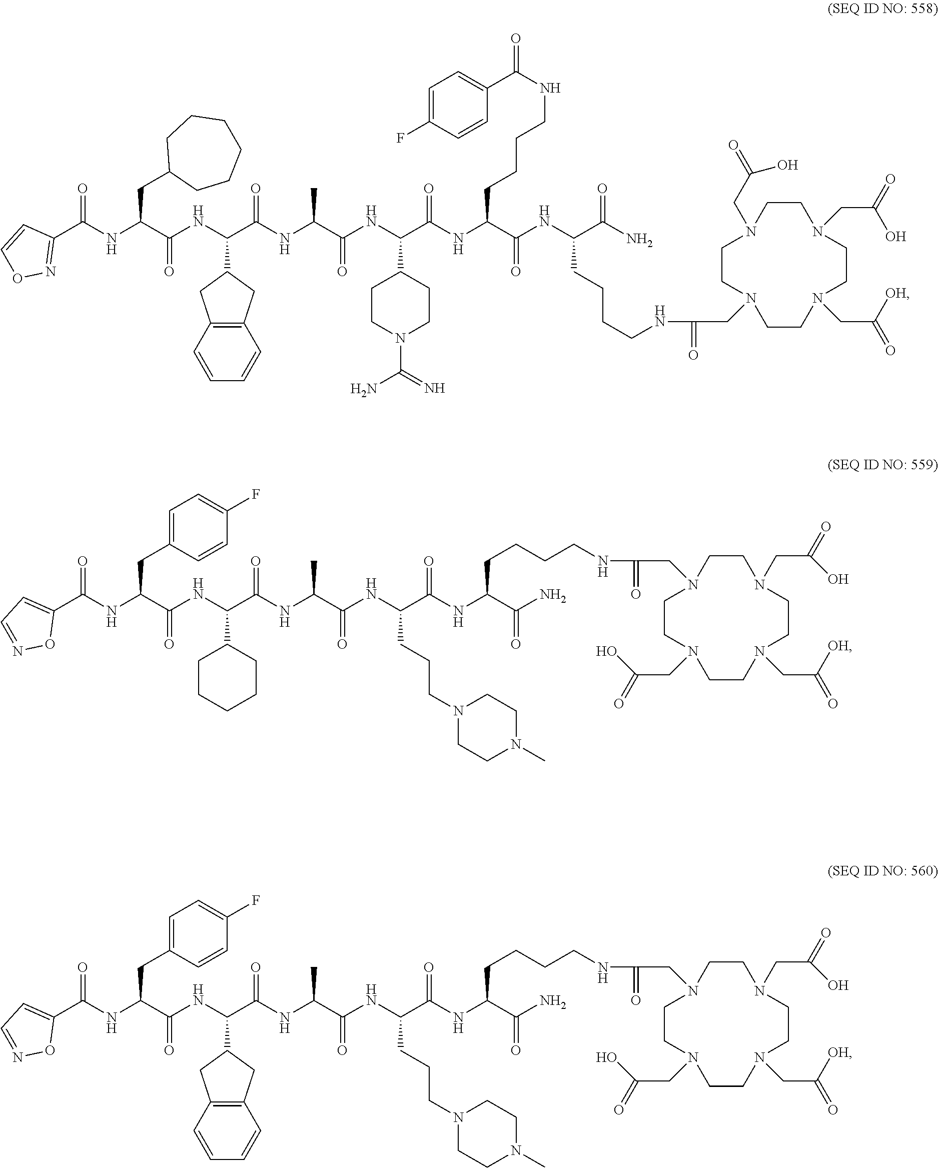 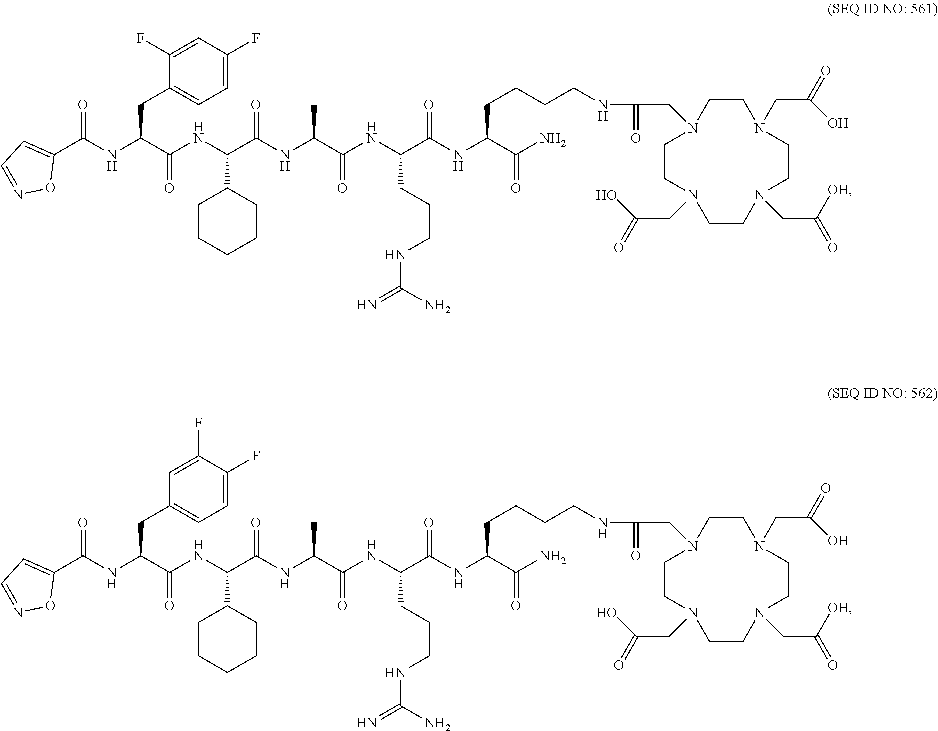 [SMILES: C[C@H](NC(=O)[C@@H](NC(=O)[C@H](Cc1ccc(F)cc1F)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O ; C[C@H](NC(=O)[C@@H](NC(=O)[C@H](Cc1ccc(F)c(F)c1)NC(=O)c1ccno1)C1CCCCC1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O] 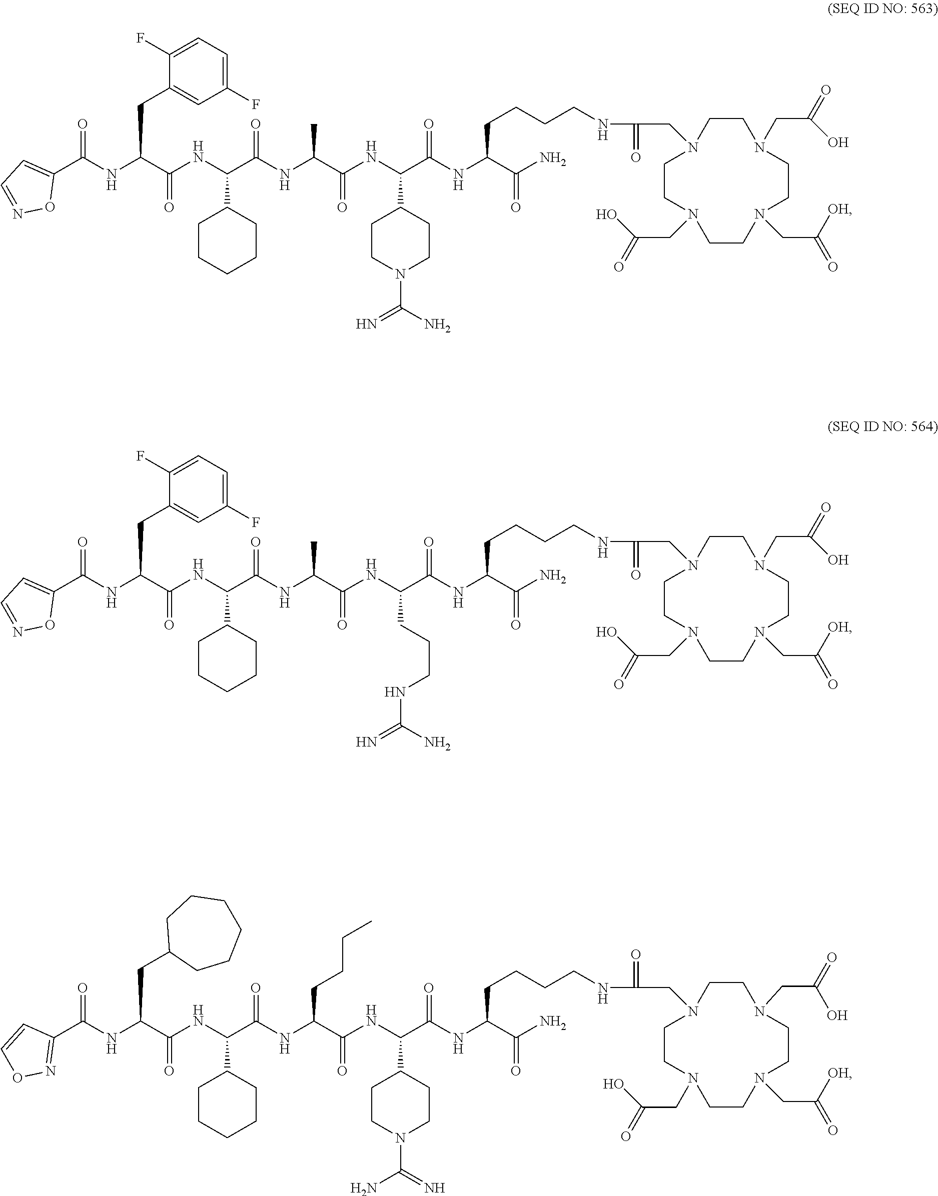 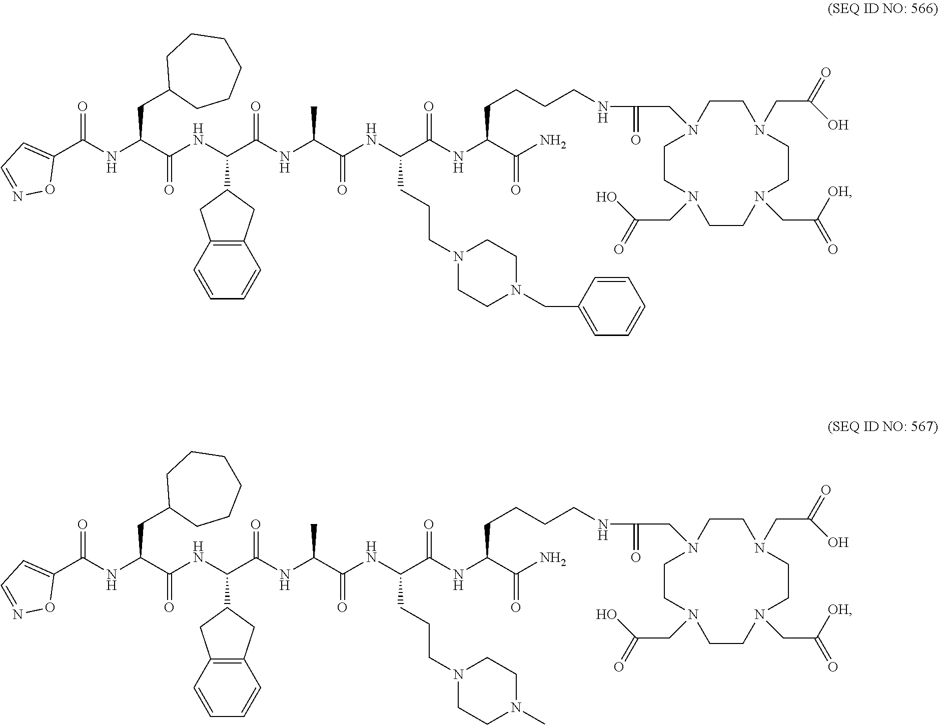 [SMILES: C[C@H](NC(=O)[C@@H](NC(=O)[C@H](CC1CCCCCC1)NC(=O)c1ccno1)C1Cc2ccccc2C1)C(=O)N[C@@H](CCCN1CCN(Cc2ccccc2)CC1)C(=O)N[C@@H](CCCCNC(=O)CN1CCN(CC(=O)O)CCN(CC(=O)O)CCN(CC(=O)O)CC1)C(N)=O ; C[C@H](NC(=O)[C@@H](NC(=O)[C@H](CC1CCCCCC1)NC(=O)c1ccno1)C1Cc2ccccc2C1)C(=O)N[C@@H](CCCN1CCN(C)CC1)C(=O)N[C@@H](CCCCNC(=O)CN1CCN(CC(=O)O)CCN(CC(=O)O)CCN(CC(=O)O)CC1)C(N)=O] 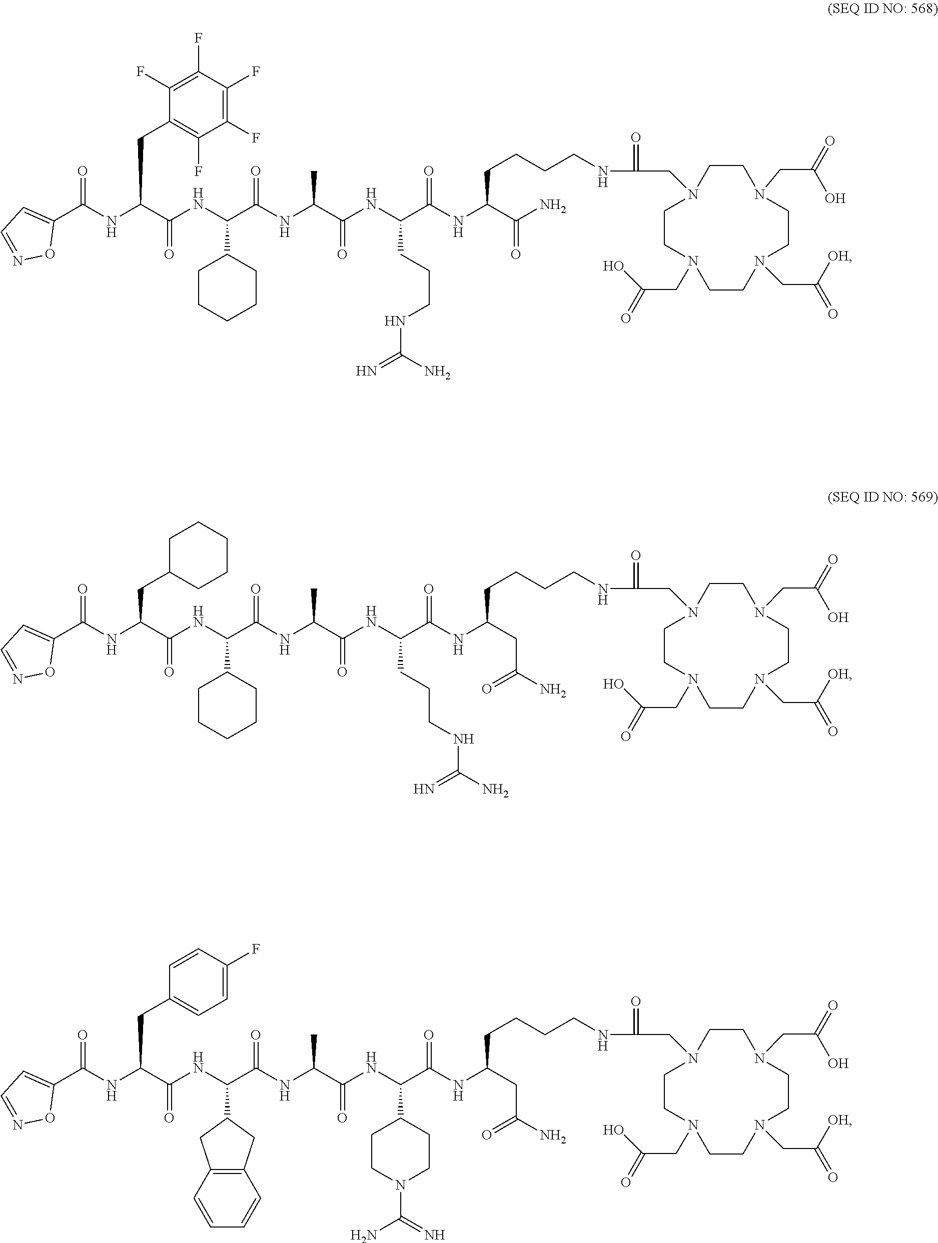 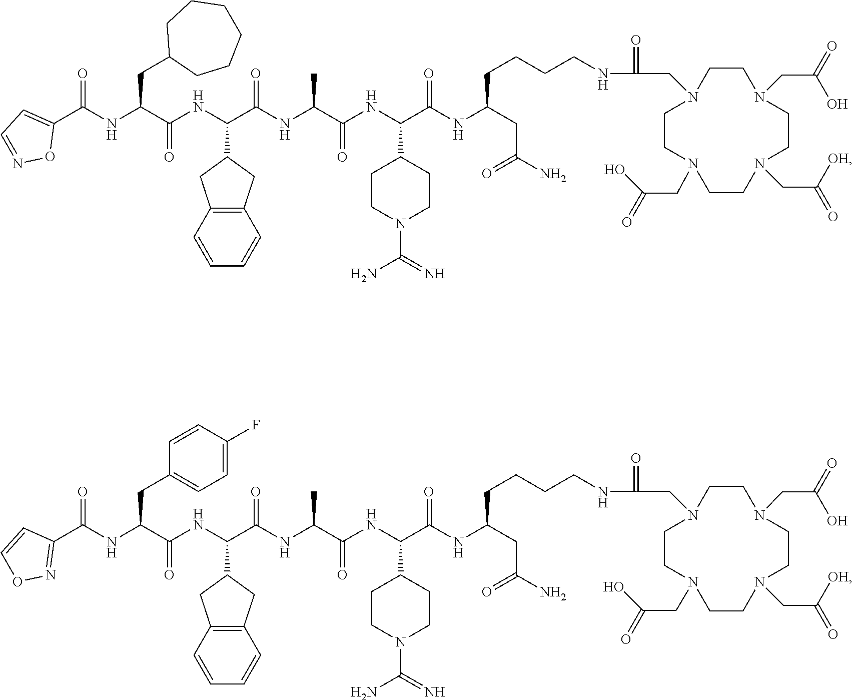 [SMILES: C[C@H](NC(=O)[C@@H](NC(=O)[C@H](CC1CCCCCC1)NC(=O)c1ccno1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)CC(N)=O)C1CCN(C(=N)N)CC1 ; C[C@H](NC(=O)[C@@H](NC(=O)[C@H](Cc1ccc(F)cc1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)CC(N)=O)C1CCN(C(=N)N)CC1] 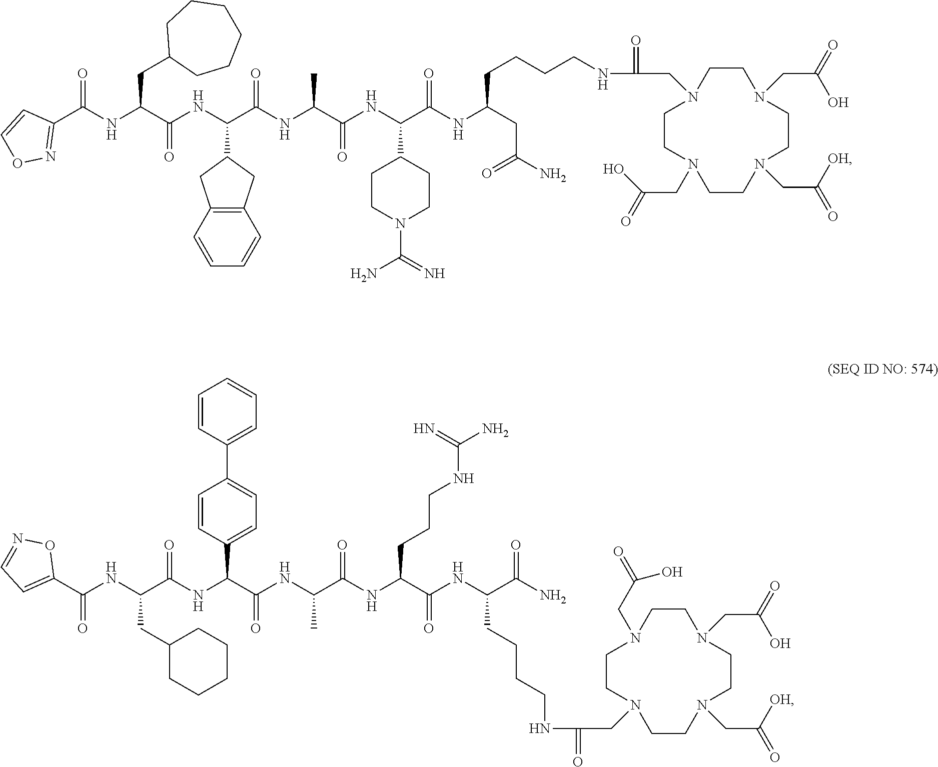 [SMILES: C[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)CC(N)=O)C1CCN(C(=N)N)CC1 ; C[C@H](NC(=O)[C@@H](NC(=O)[C@H](CC1CCCCC1)NC(=O)c1ccno1)c1ccc(-c2ccccc2)cc1)C(=O)N[C@@H](CCCNC(=N)N)C(=O)N[C@@H](CCCCNC(=O)CN1CCN(CC(=O)O)CCN(CC(=O)O)CCN(CC(=O)O)CC1)C(N)=O] 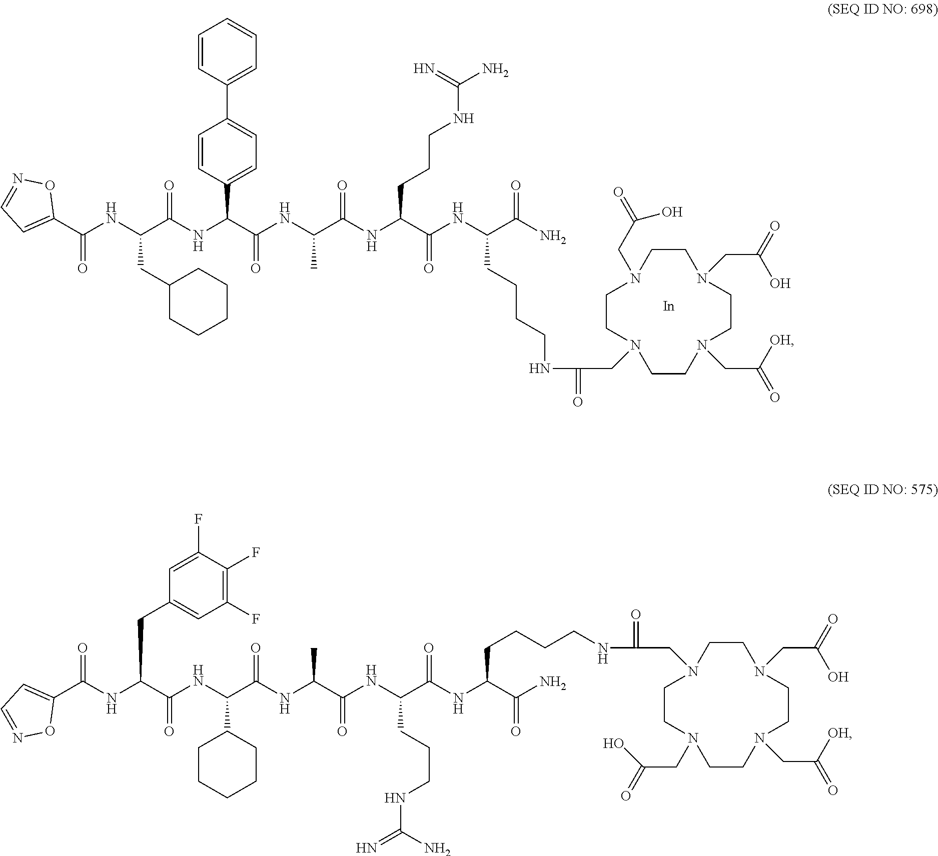 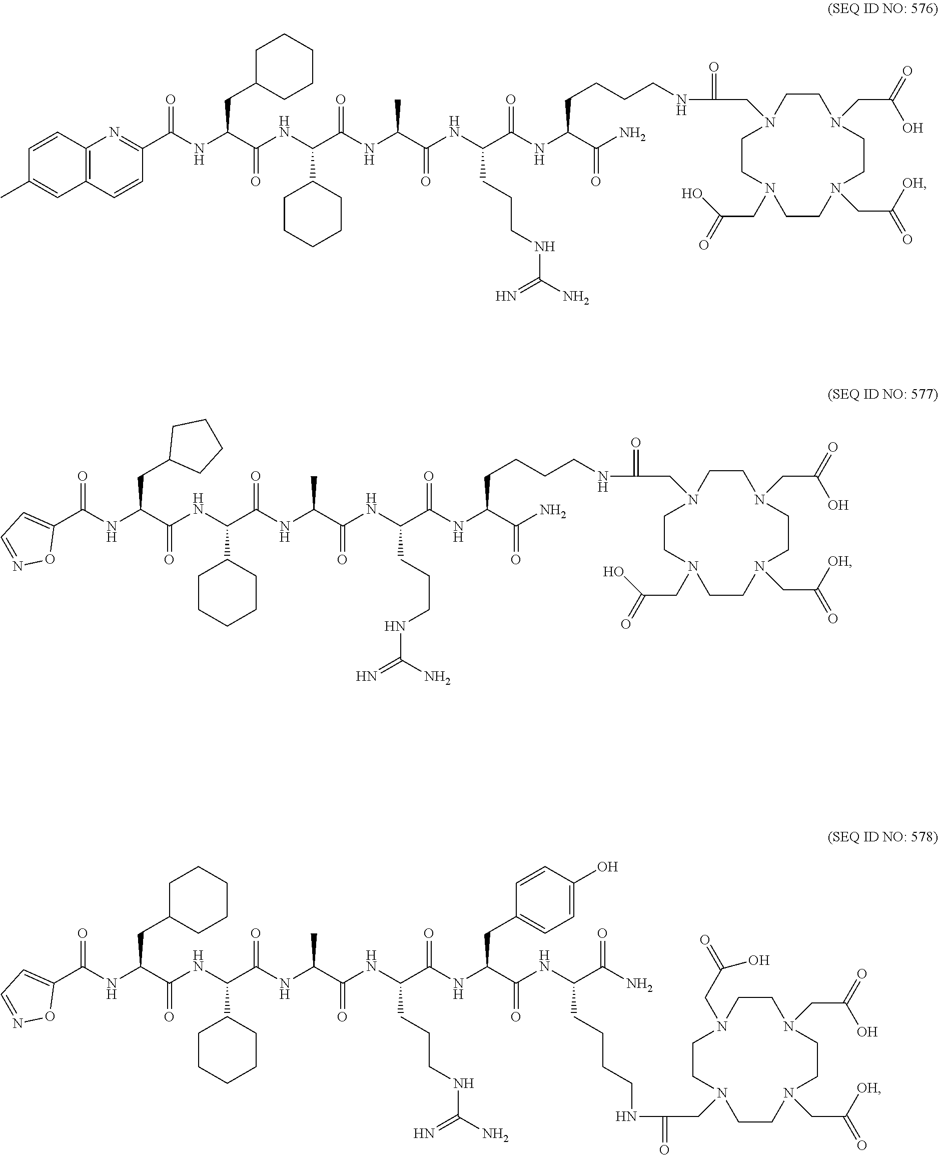 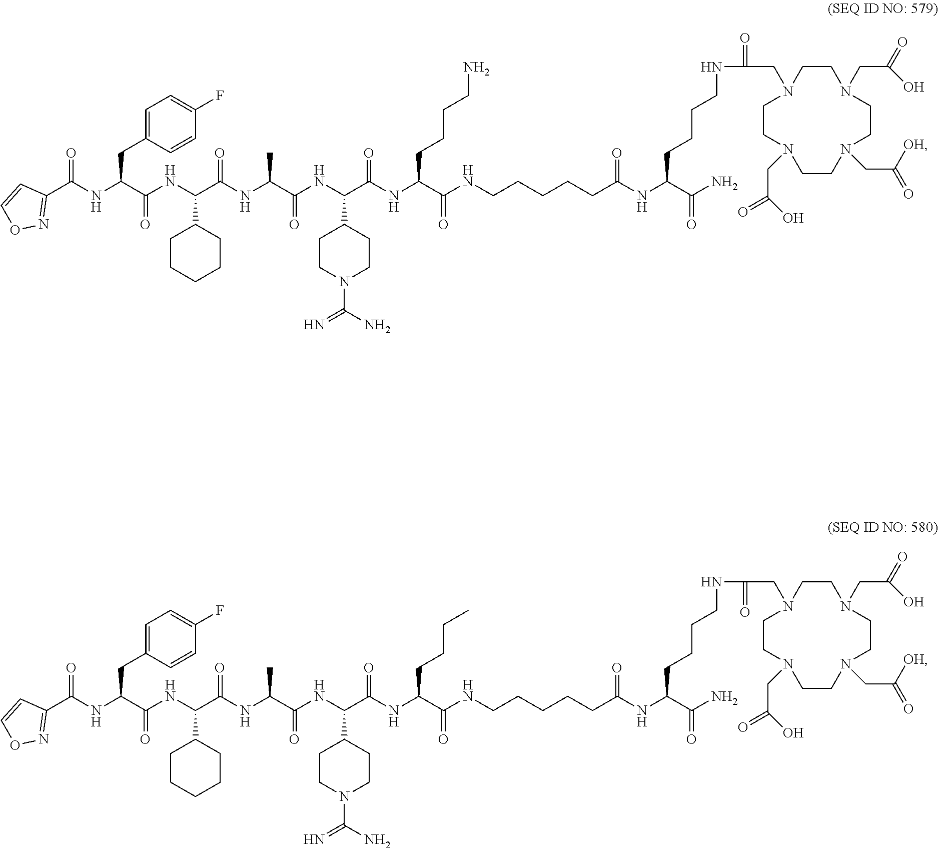 [SMILES: C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCN)C(=O)NCCCCCC(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; CCCC[C@H](NC(=O)[C@@H](NC(=O)[C@H](C)NC(=O)[C@@H](NC(=O)[C@H](Cc1ccc(F)cc1)NC(=O)c1ccon1)C1CCCCC1)C1CCN(C(=N)N)CC1)C(=O)NCCCCCC(=O)N[C@@H](CCCCNC(=O)CN1CCN(CC(=O)O)CCN(CC(=O)O)CCN(CC(=O)O)CC1)C(N)=O] 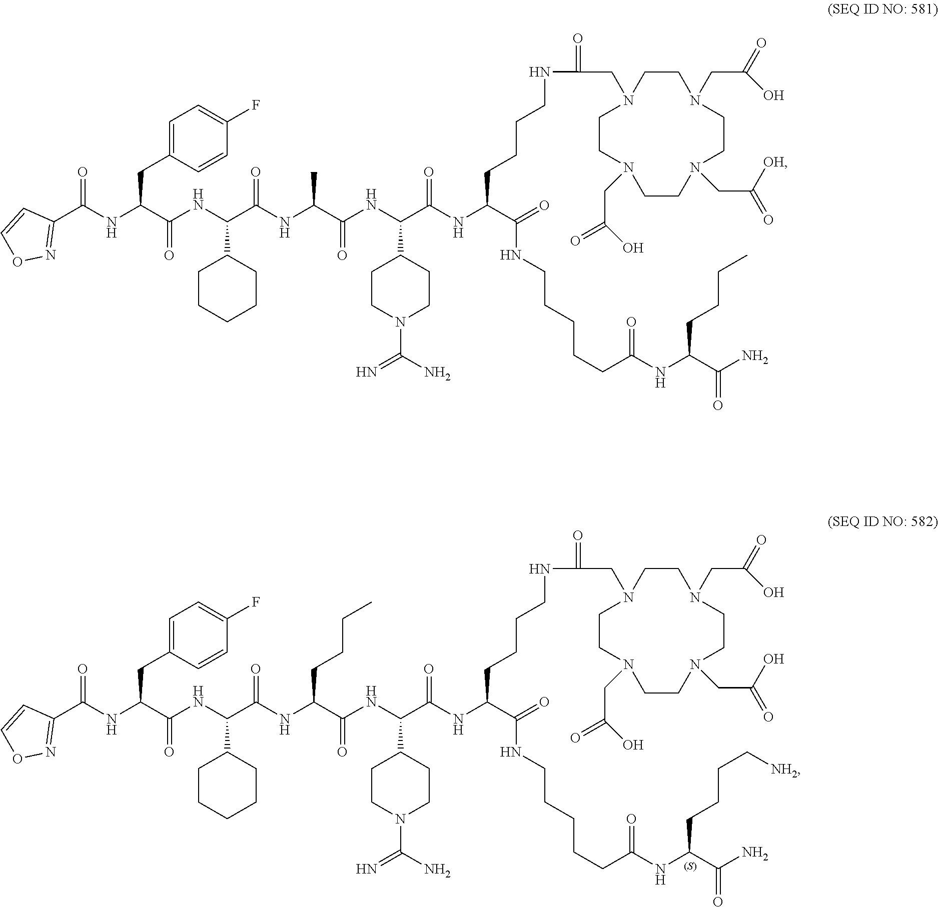 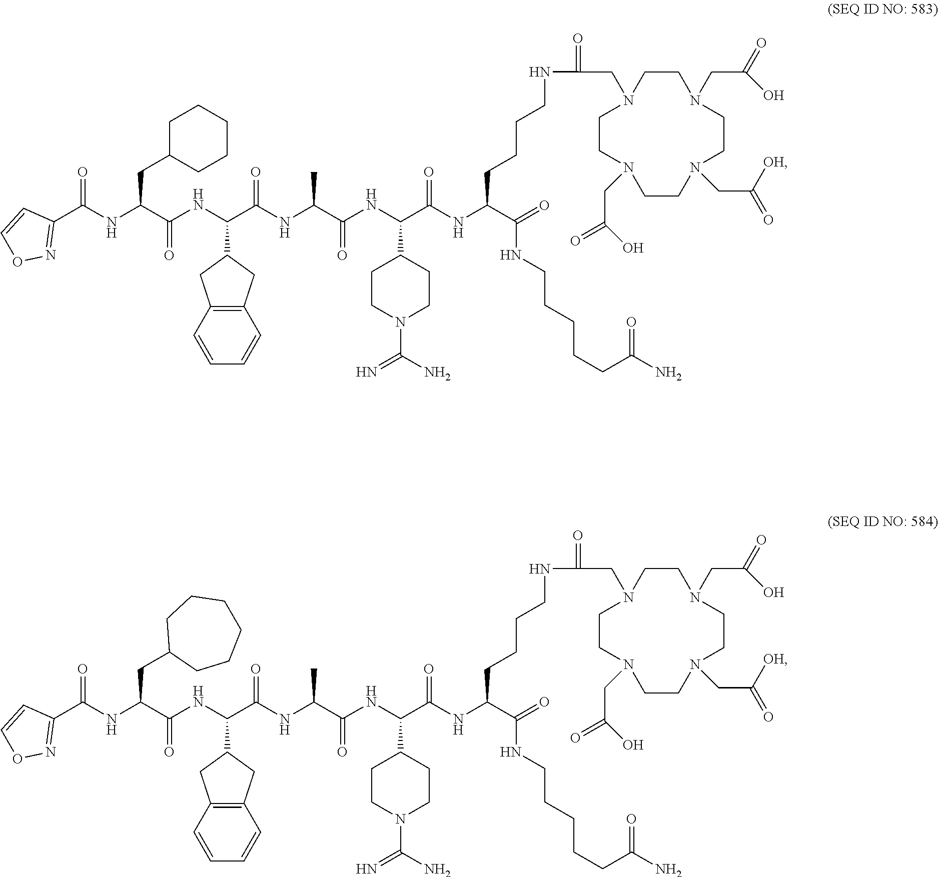 [SMILES: C[C@H](NC(=O)[C@@H](NC(=O)[C@H](CC1CCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1 ; C[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1] 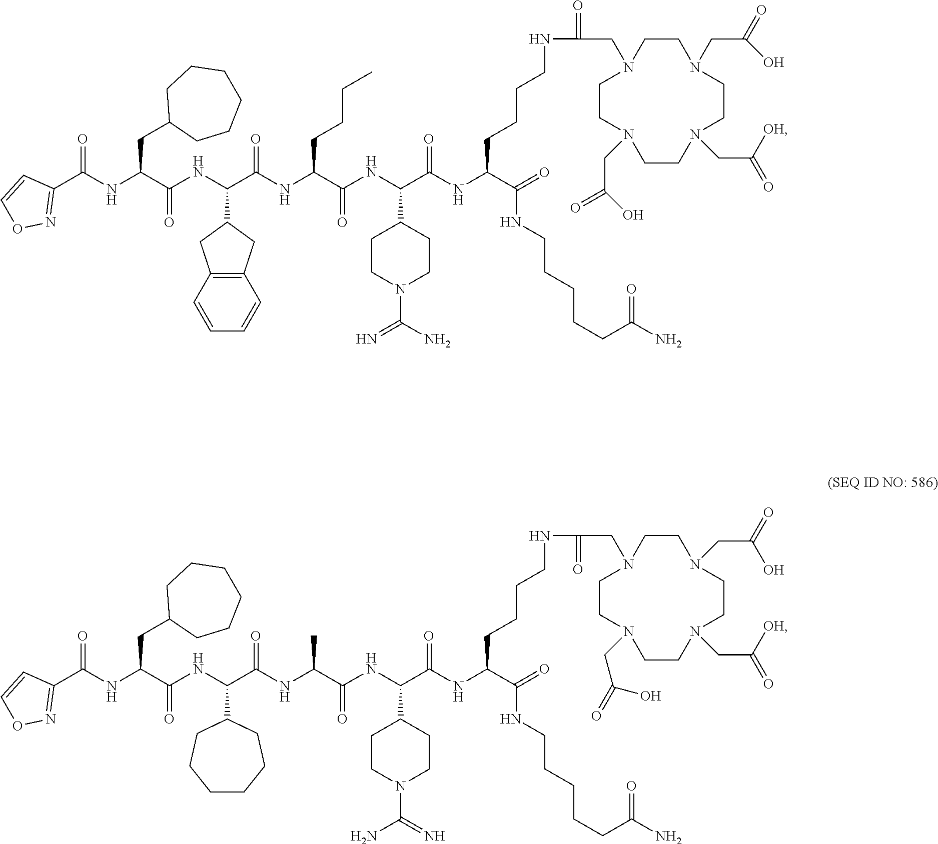 [SMILES: CCCC[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1 ; C[C@H](NC(=O)[C@@H](NC(=O)[C@H](CC1CCCCCC1)NC(=O)c1ccon1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1] 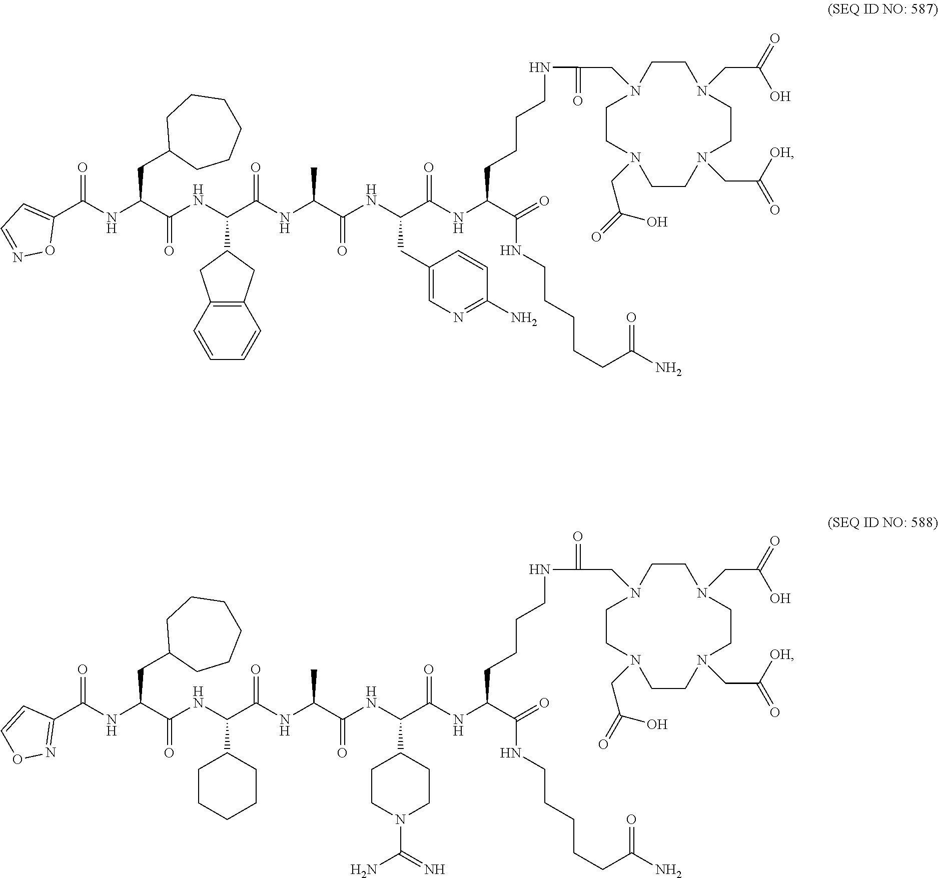 [SMILES: C[C@H](NC(=O)[C@@H](NC(=O)[C@H](CC1CCCCCC1)NC(=O)c1ccno1)C1Cc2ccccc2C1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(=O)NCCCCCC(N)=O ; C[C@H](NC(=O)[C@@H](NC(=O)[C@H](CC1CCCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(N)=O)C1CCN(C(=N)N)CC1] 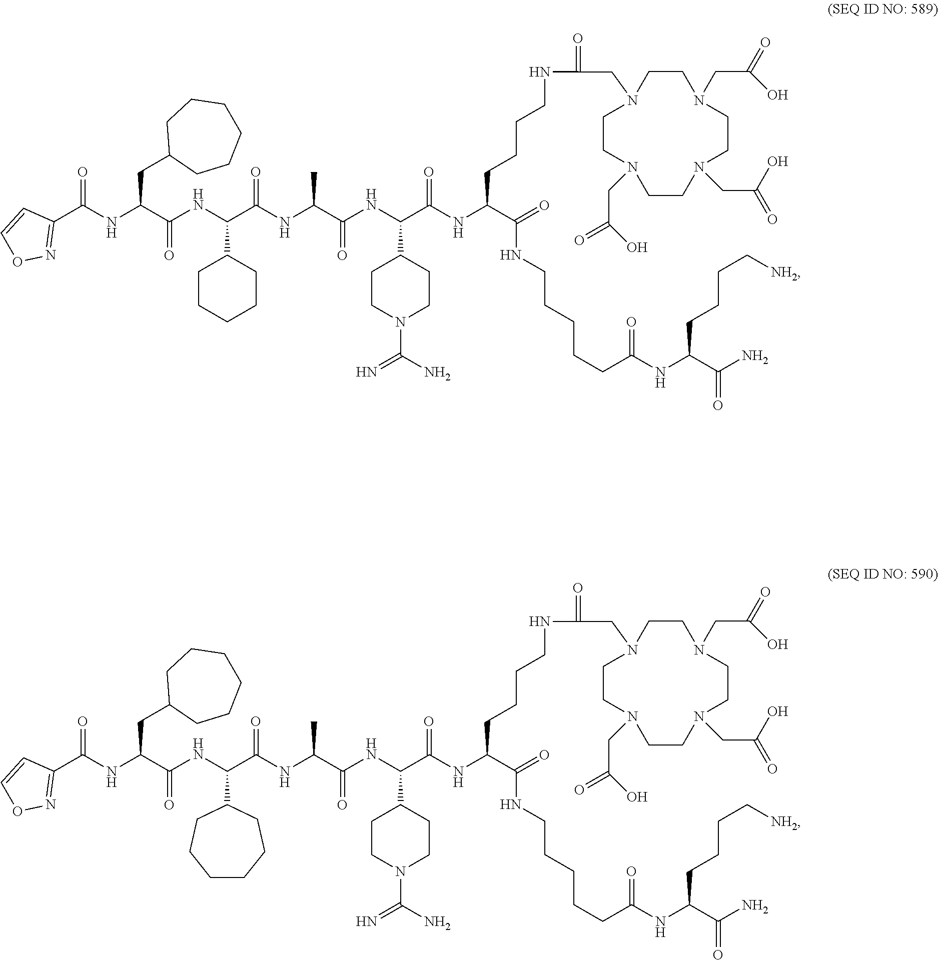 [SMILES: C[C@H](NC(=O)[C@@H](NC(=O)[C@H](CC1CCCCCC1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1 ; C[C@H](NC(=O)[C@@H](NC(=O)[C@H](CC1CCCCCC1)NC(=O)c1ccon1)C1CCCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1] 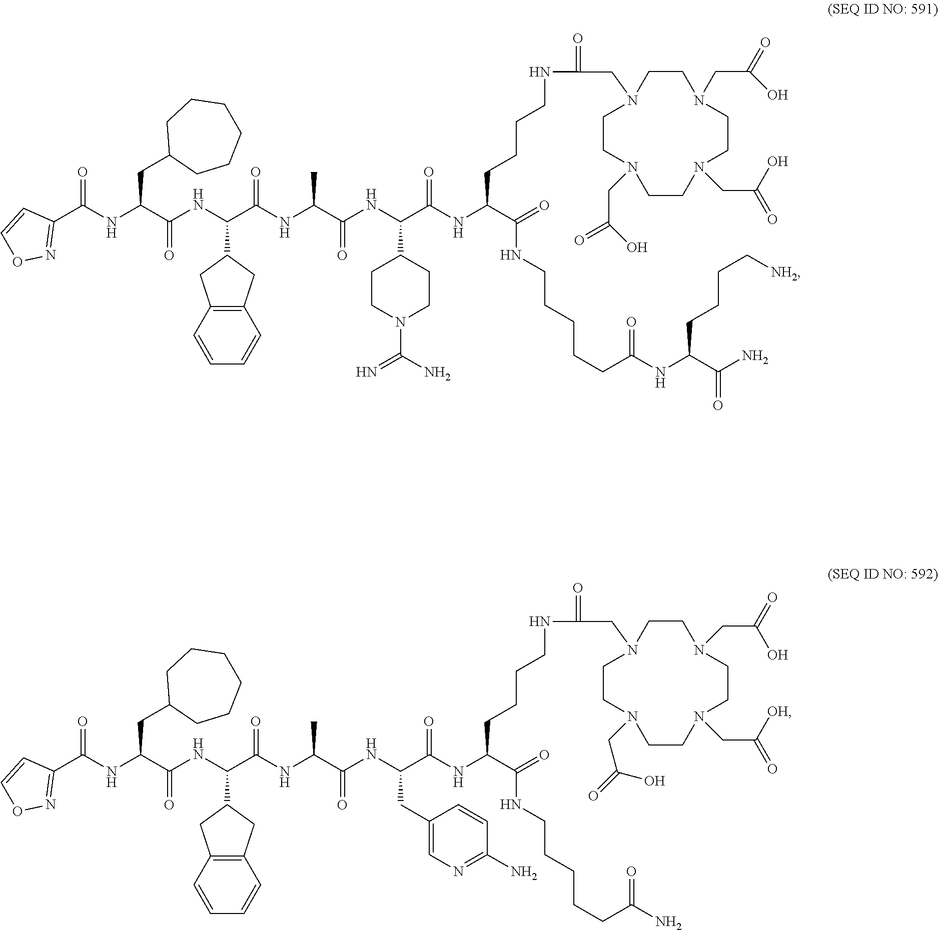 [SMILES: C[C@H](NC(=O)[C@@H](NC(=O)[C@H](CC1CCCCCC1)NC(=O)c1ccon1)C1Cc2ccccc2C1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1 ; C[C@H](NC(=O)[C@@H](NC(=O)[C@H](CC1CCCCCC1)NC(=O)c1ccon1)C1Cc2ccccc2C1)C(=O)N[C@@H](Cc1ccc(N)nc1)C(=O)N[C@@H](CCCCNC(=O)CN1CCN(CC(=O)O)CCN(CC(=O)O)CCN(CC(=O)O)CC1)C(=O)NCCCCCC(N)=O] 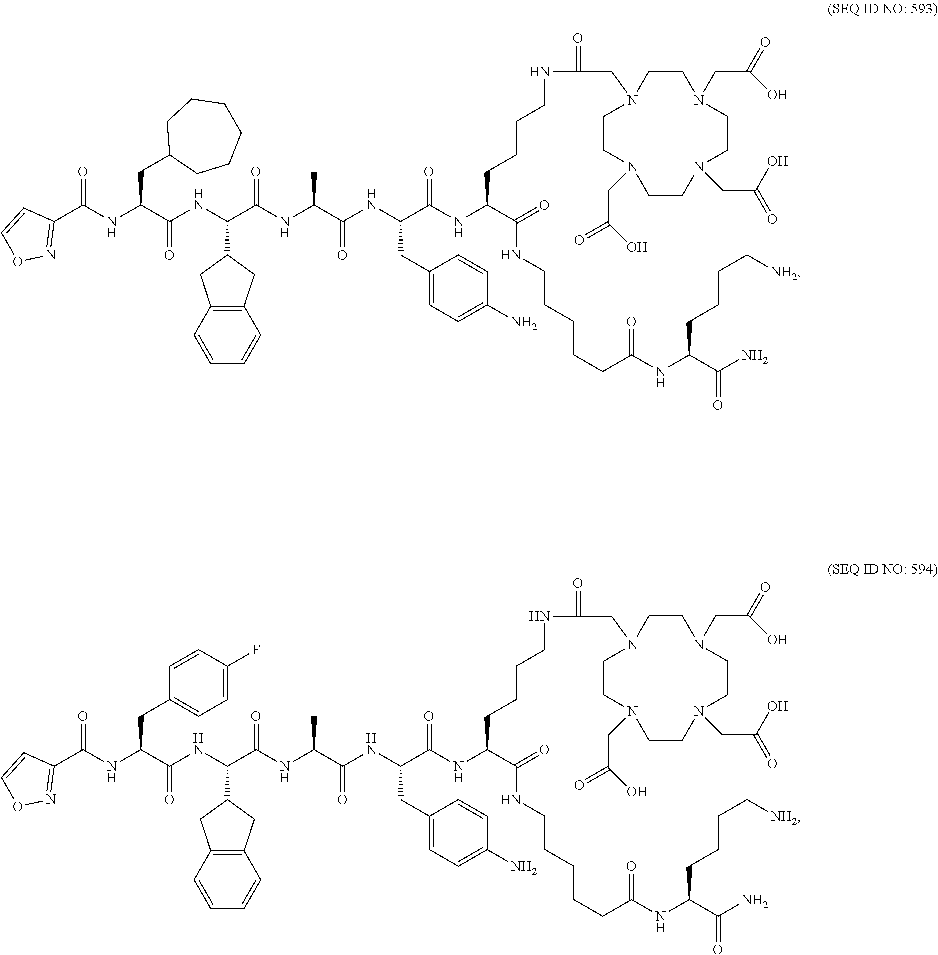 [SMILES: C[C@H](NC(=O)[C@@H](NC(=O)[C@H](CC1CCCCCC1)NC(=O)c1ccon1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O ; C[C@H](NC(=O)[C@@H](NC(=O)[C@H](Cc1ccc(F)cc1)NC(=O)c1ccon1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O] 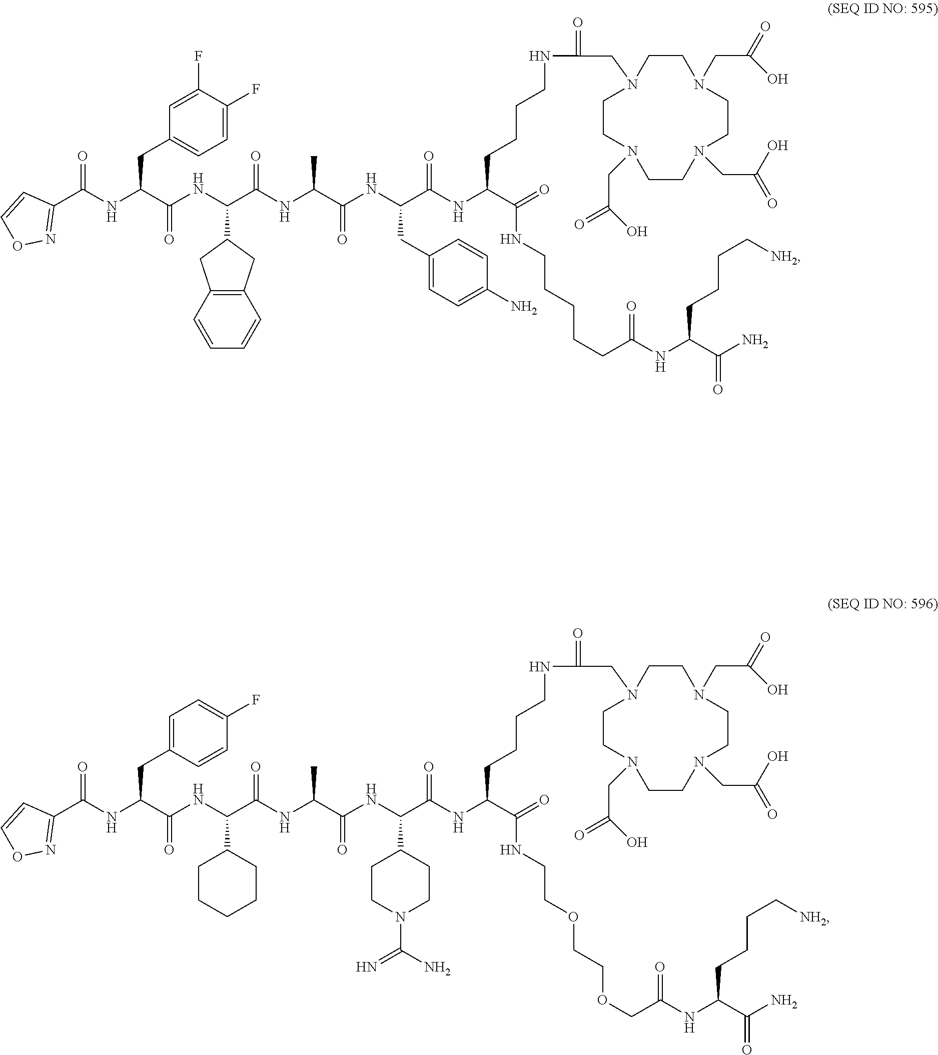 [SMILES: C[C@H](NC(=O)[C@@H](NC(=O)[C@H](Cc1ccc(F)c(F)c1)NC(=O)c1ccon1)C1Cc2ccccc2C1)C(=O)N[C@@H](Cc1ccc(N)cc1)C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O ; C[C@H](NC(=O)[C@@H](NC(=O)[C@H](Cc1ccc(F)cc1)NC(=O)c1ccon1)C1CCCCC1)C(=O)N[C@H](C(=O)N[C@@H](CCCCNC(=O)CN1CCN(CC(=O)O)CCN(CC(=O)O)CCN(CC(=O)O)CC1)C(=O)NCCOCCOCC(=O)N[C@@H](CCCCN)C(N)=O)C1CCN(C(=N)N)CC1] 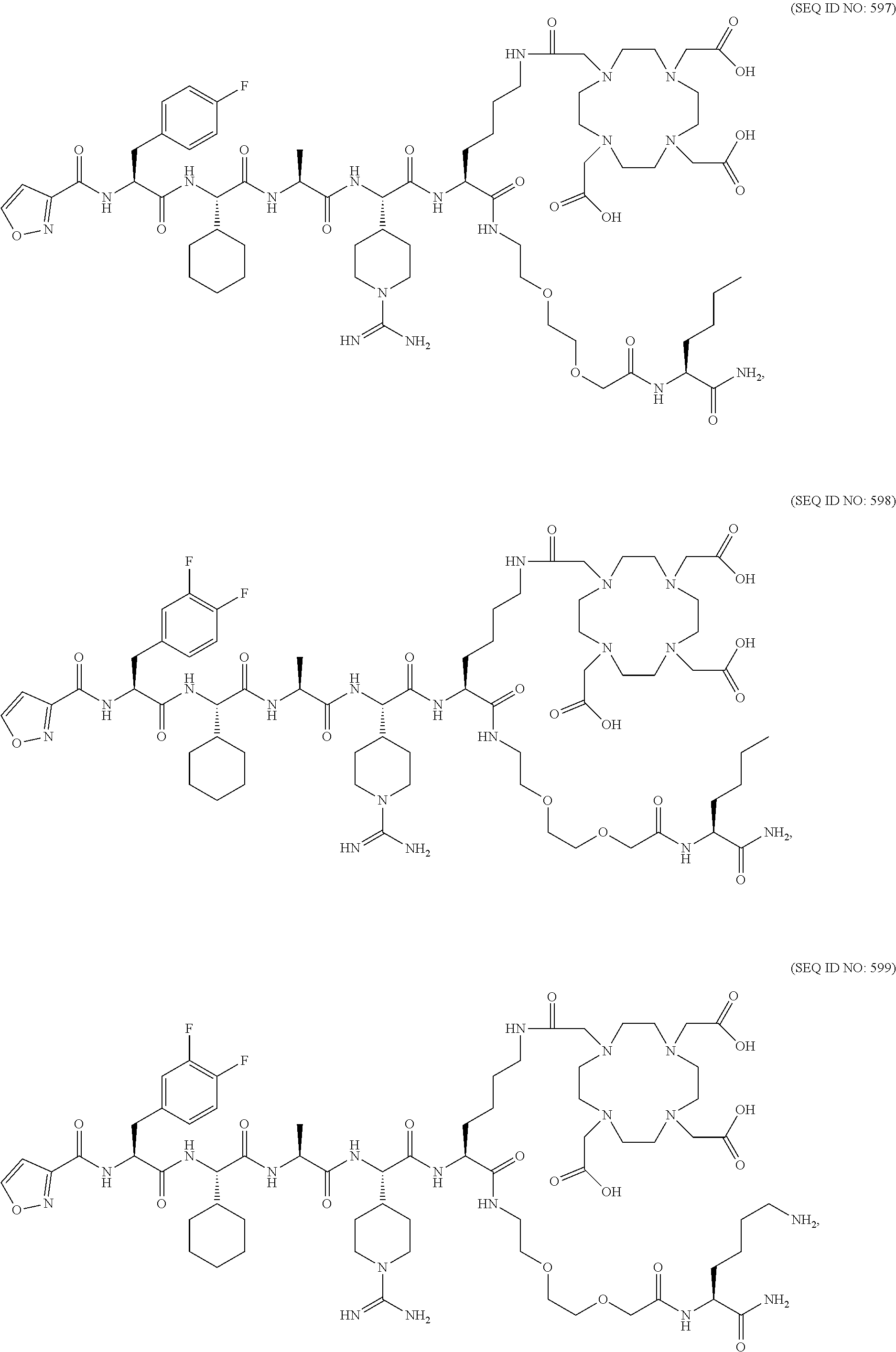 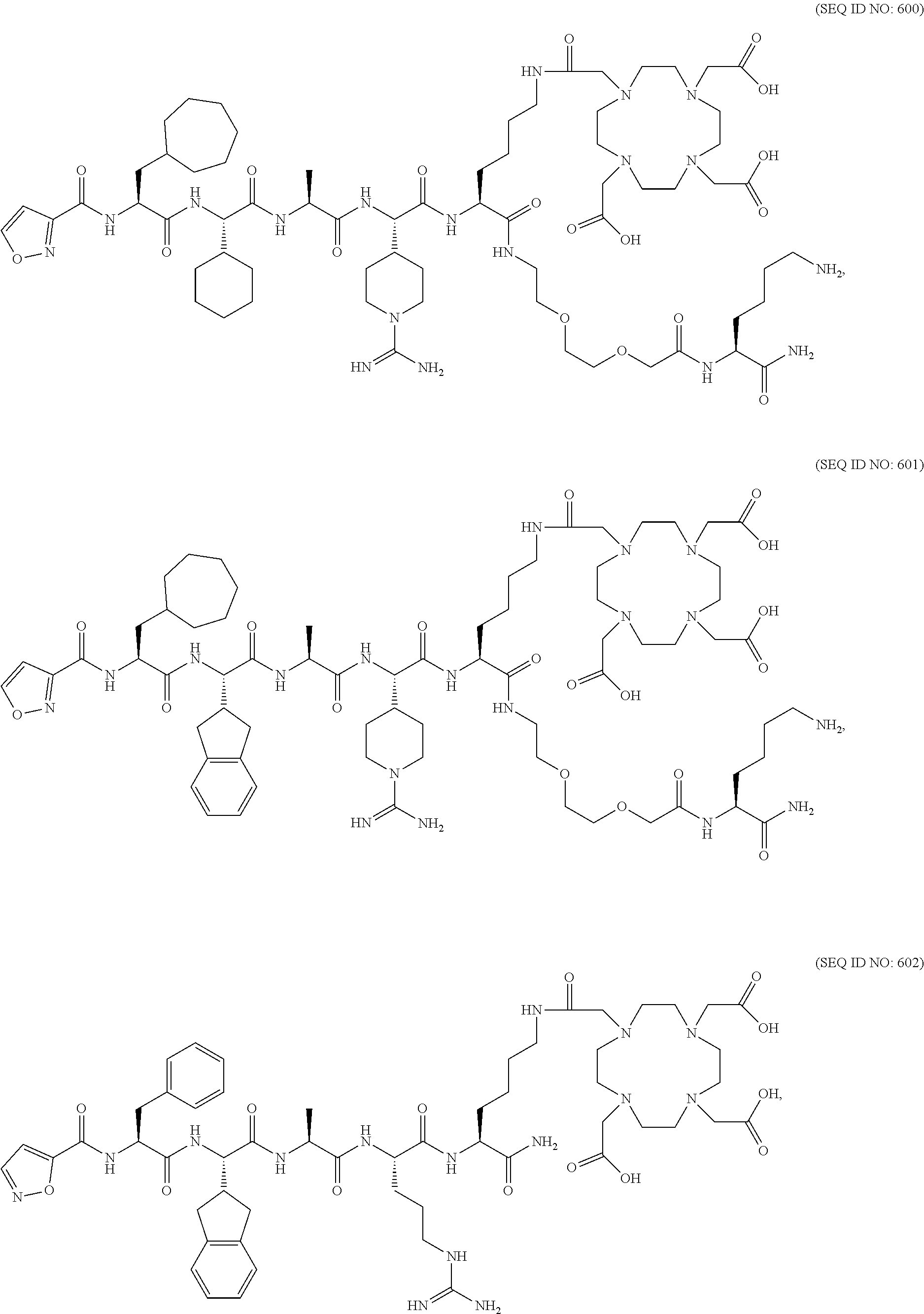 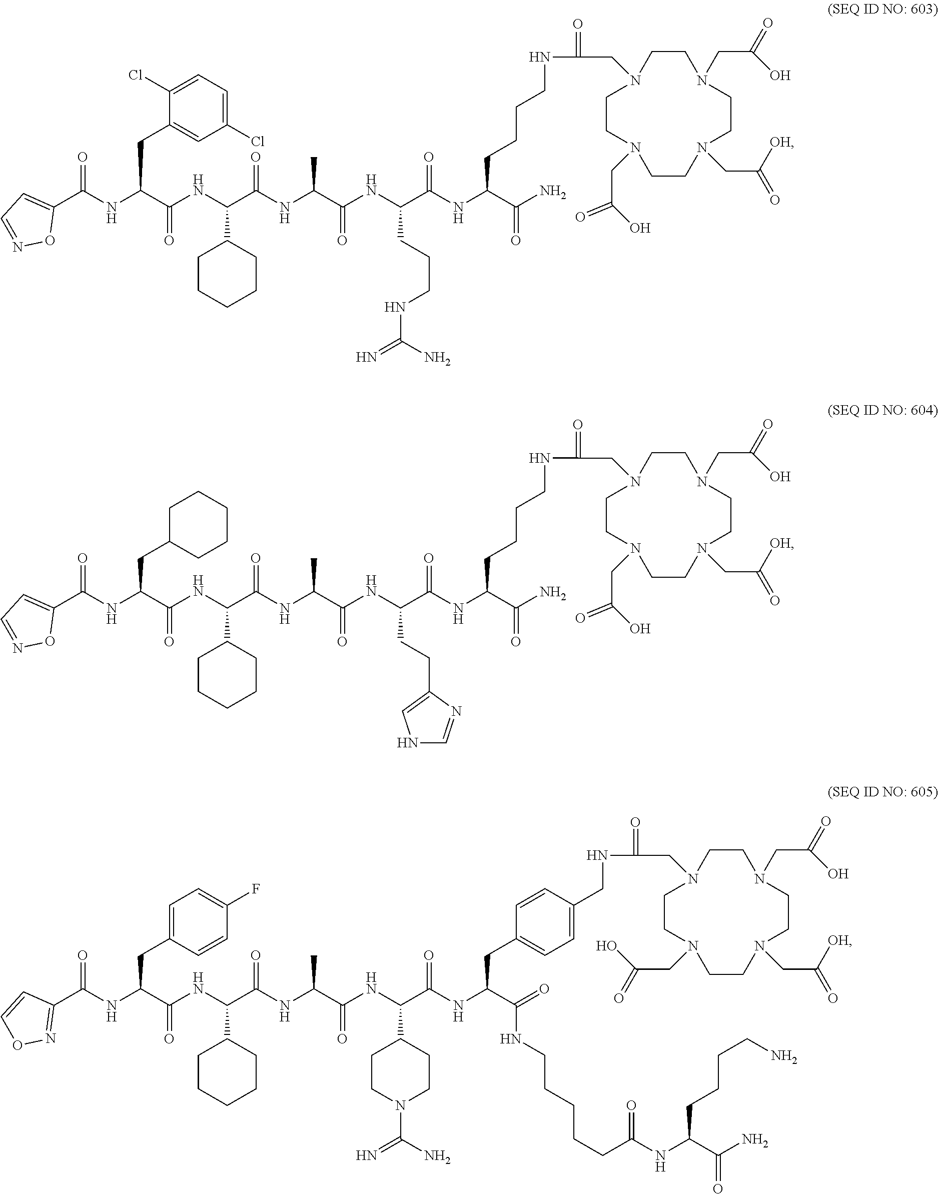 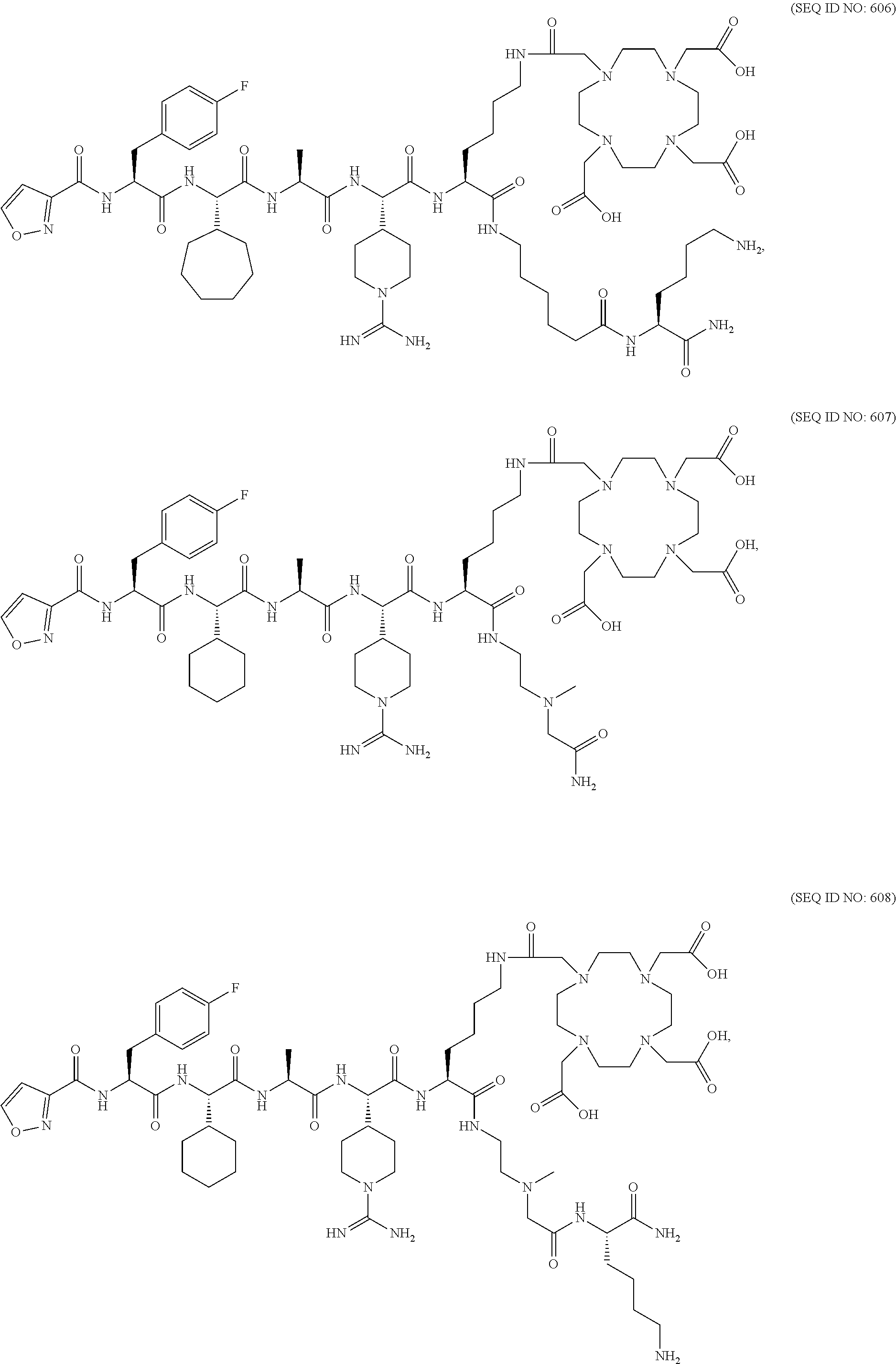 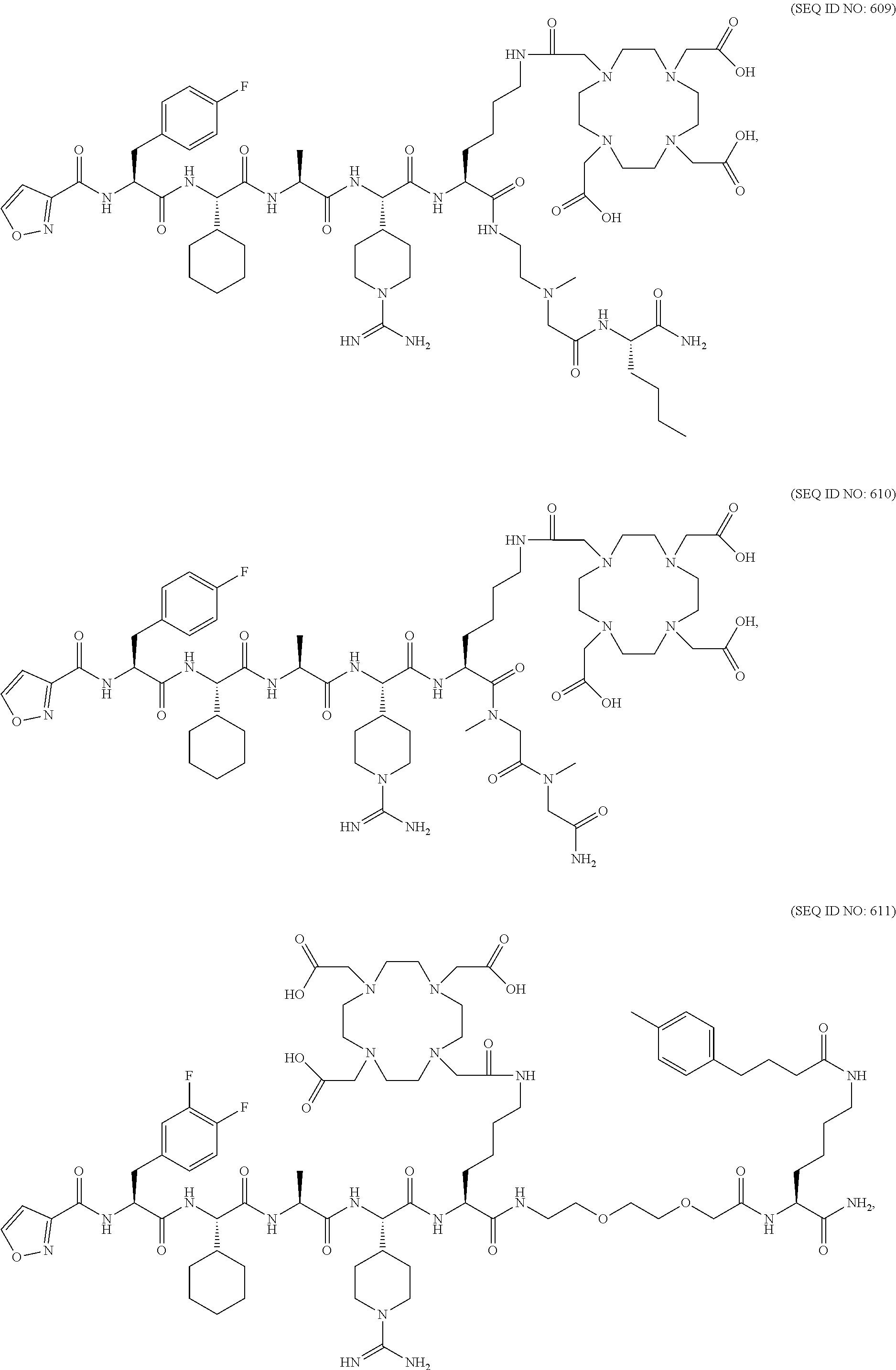 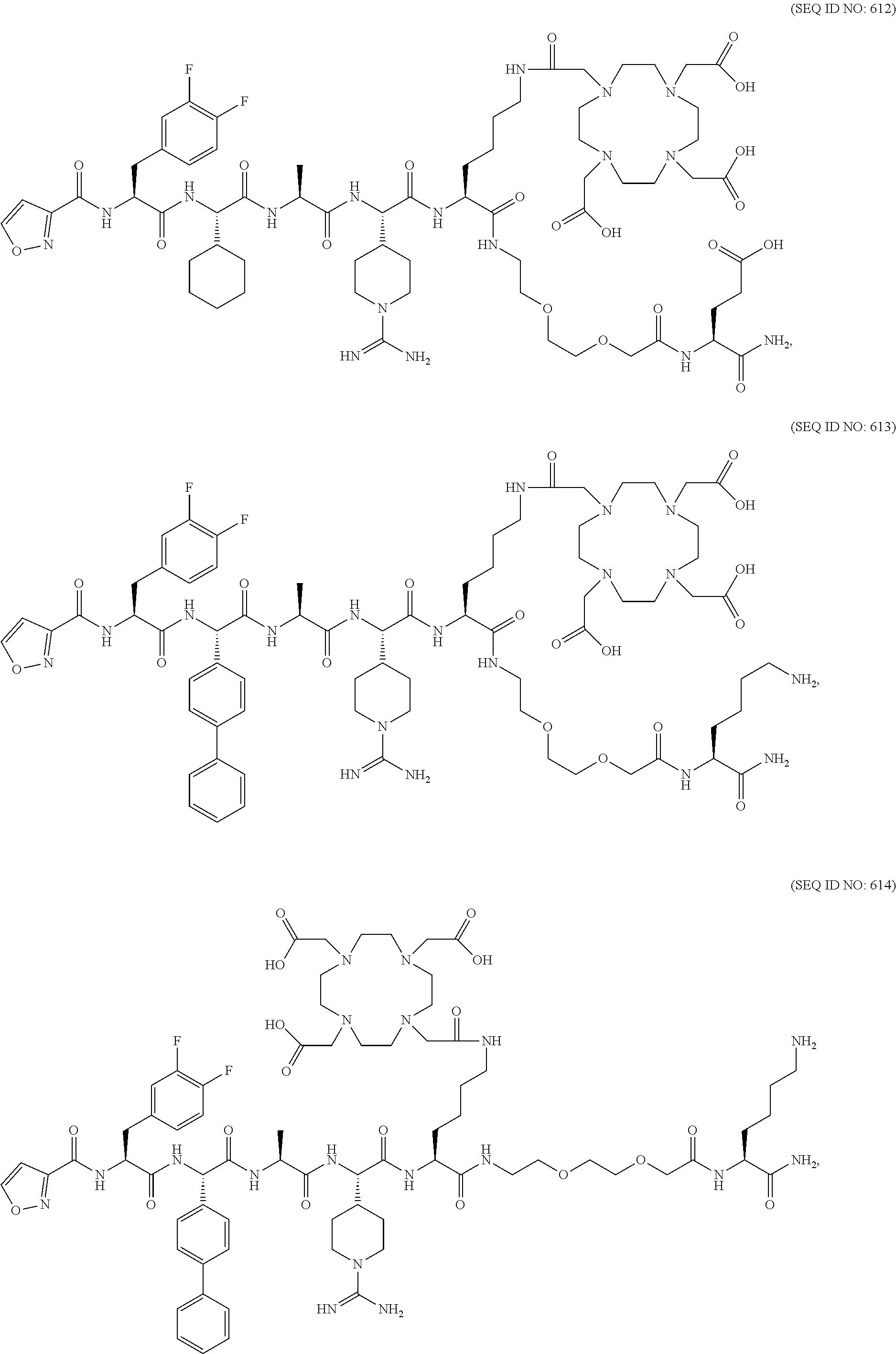 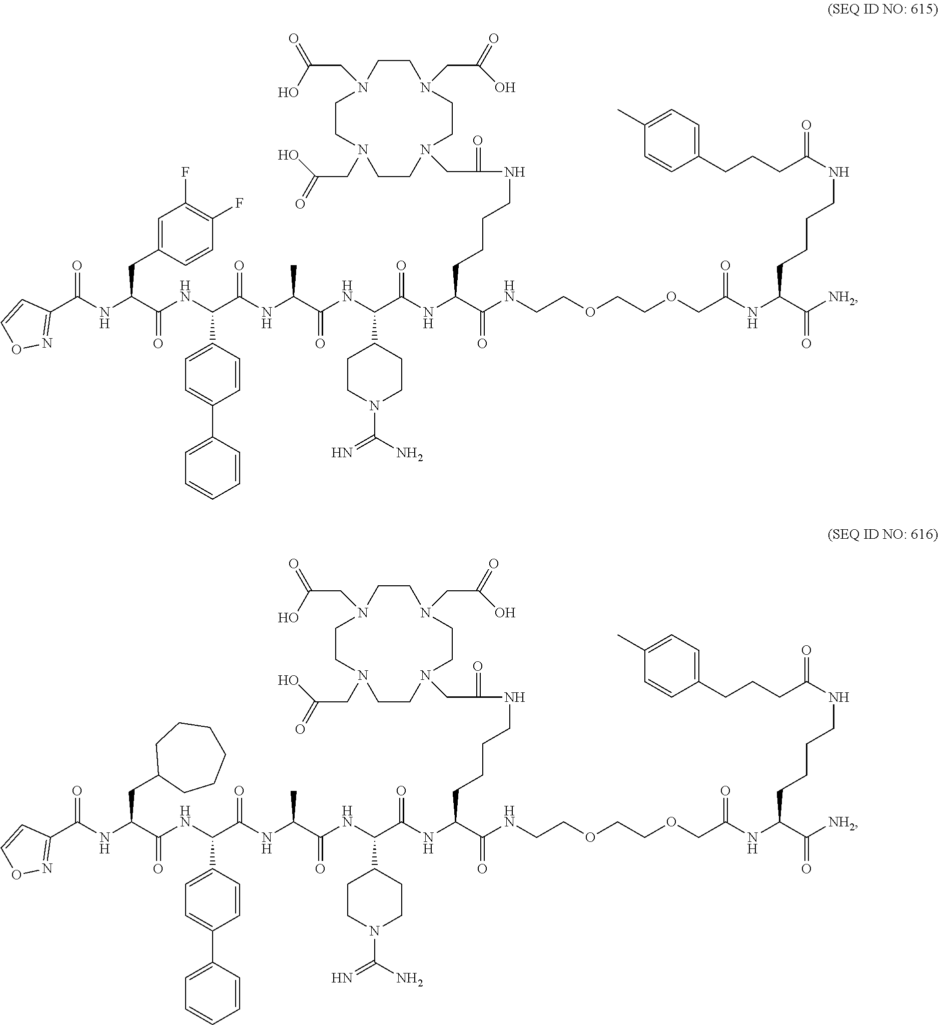 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)c2ccc(-c3ccccc3)cc2)C2CCN(C(=N)N)CC2)C(N)=O)cc1 ; Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](CC2CCCCCC2)NC(=O)c2ccon2)c2ccc(-c3ccccc3)cc2)C2CCN(C(=N)N)CC2)C(N)=O)cc1] 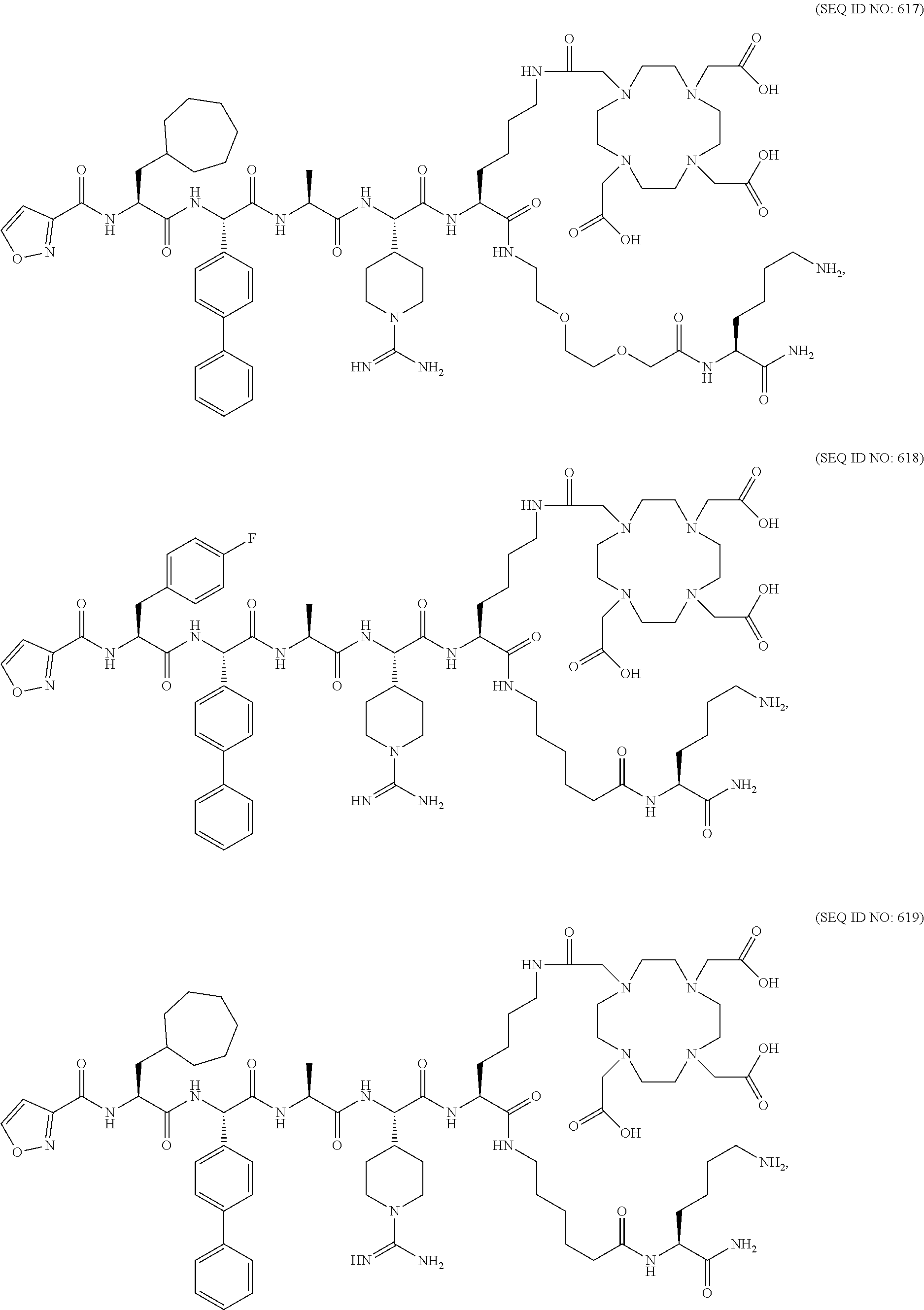 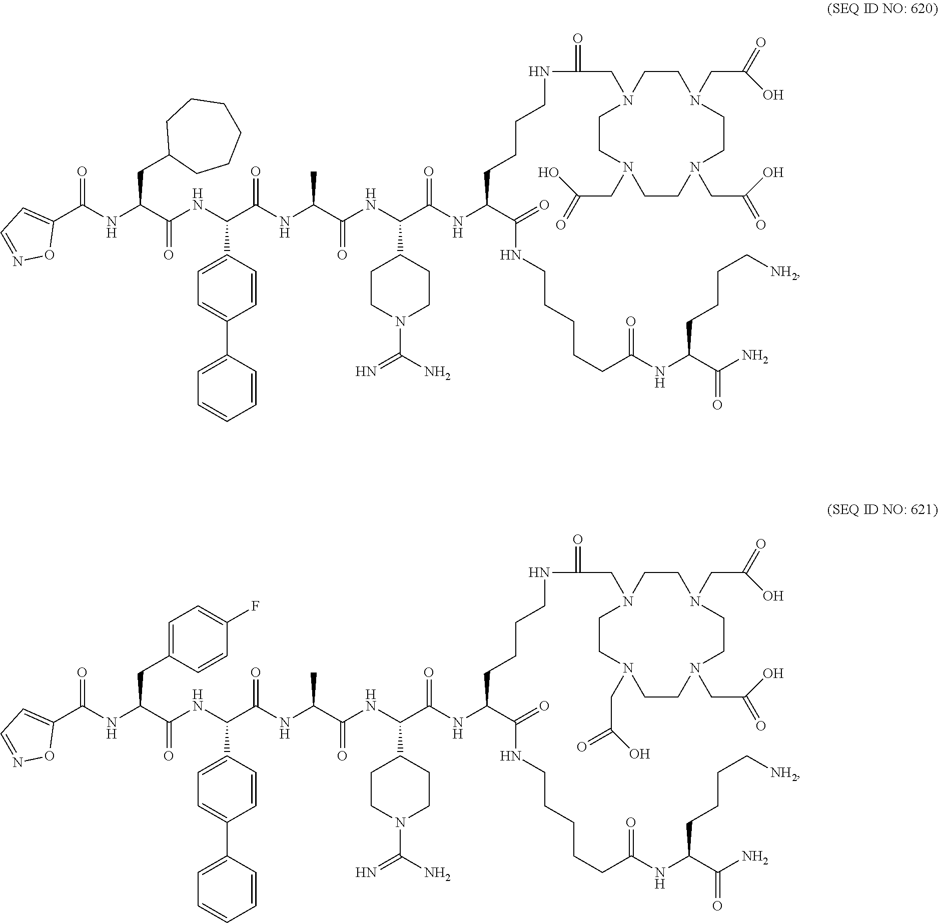 [SMILES: C[C@H](NC(=O)[C@@H](NC(=O)[C@H](CC1CCCCCC1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1 ; C[C@H](NC(=O)[C@@H](NC(=O)[C@H](Cc1ccc(F)cc1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1] 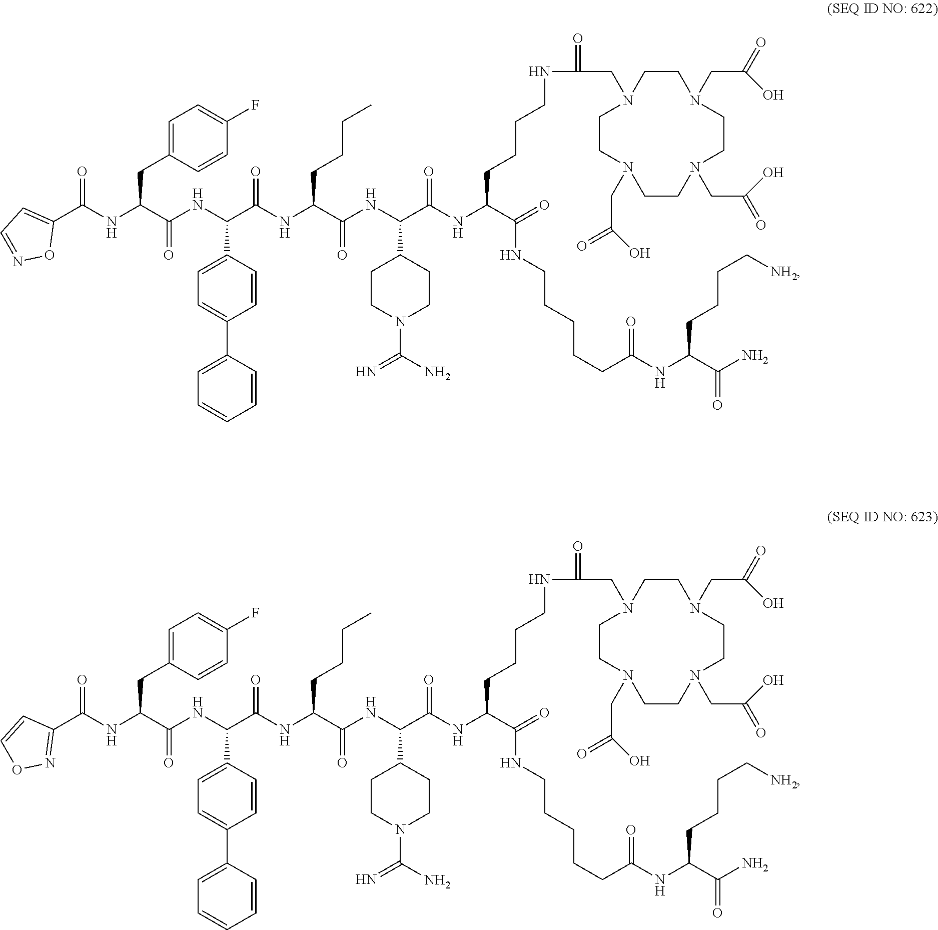 [SMILES: CCCC[C@H](NC(=O)[C@@H](NC(=O)[C@H](Cc1ccc(F)cc1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1 ; CCCC[C@H](NC(=O)[C@@H](NC(=O)[C@H](Cc1ccc(F)cc1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1] 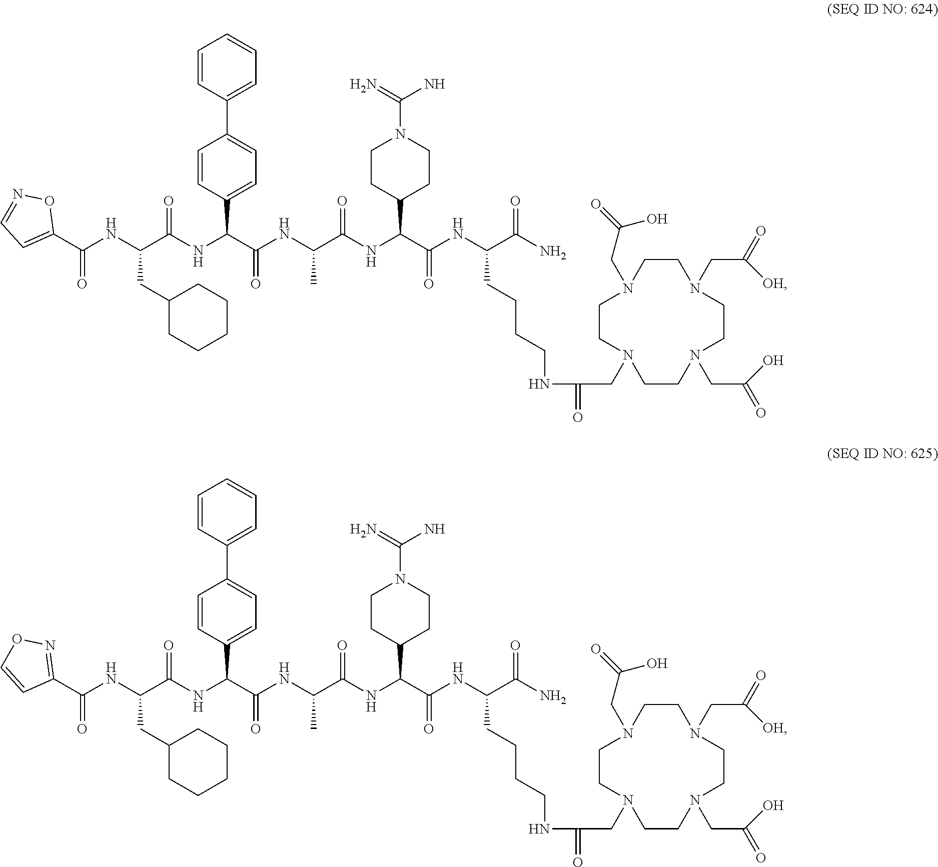 [SMILES: C[C@H](NC(=O)[C@@H](NC(=O)[C@H](CC1CCCCC1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](CC1CCCCC1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1] 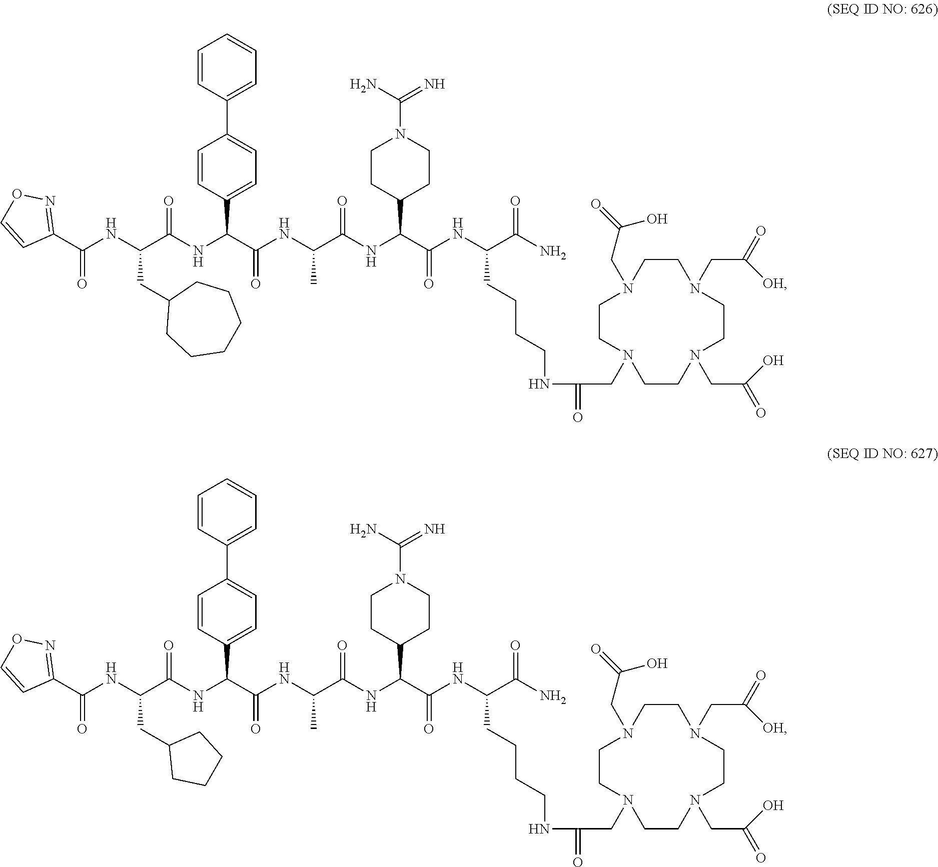 [SMILES: C[C@H](NC(=O)[C@@H](NC(=O)[C@H](CC1CCCCCC1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](CC1CCCC1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1] 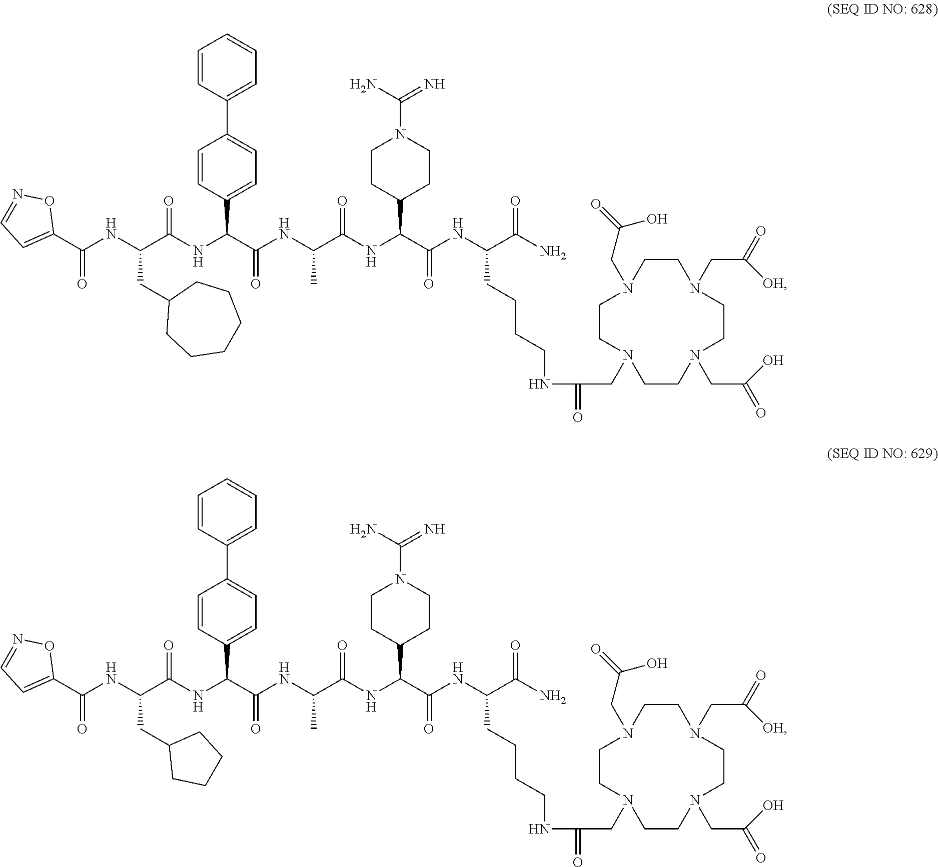 [SMILES: C[C@H](NC(=O)[C@@H](NC(=O)[C@H](CC1CCCCCC1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](CC1CCCC1)NC(=O)c1ccno1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(N)=O)C1CCN(C(=N)N)CC1] 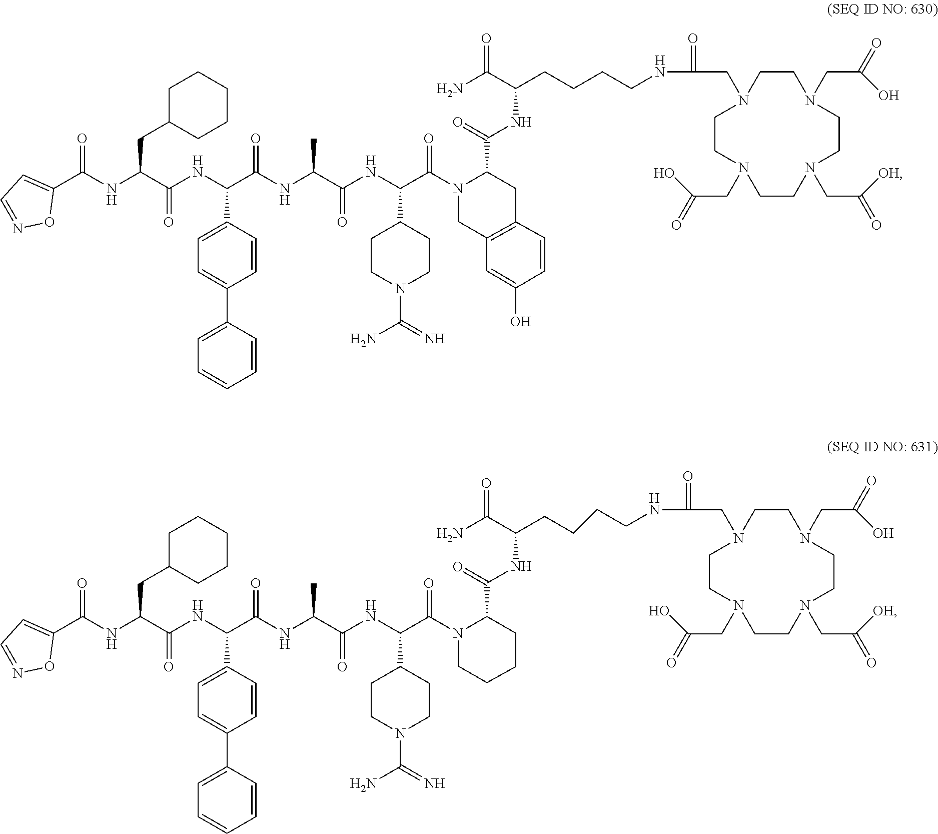 [SMILES: C[C@H](NC(=O)[C@@H](NC(=O)[C@H](CC1CCCCC1)NC(=O)c1ccno1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](CC1CCCCC1)NC(=O)c1ccno1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(N)=O)C1CCN(C(=N)N)CC1] 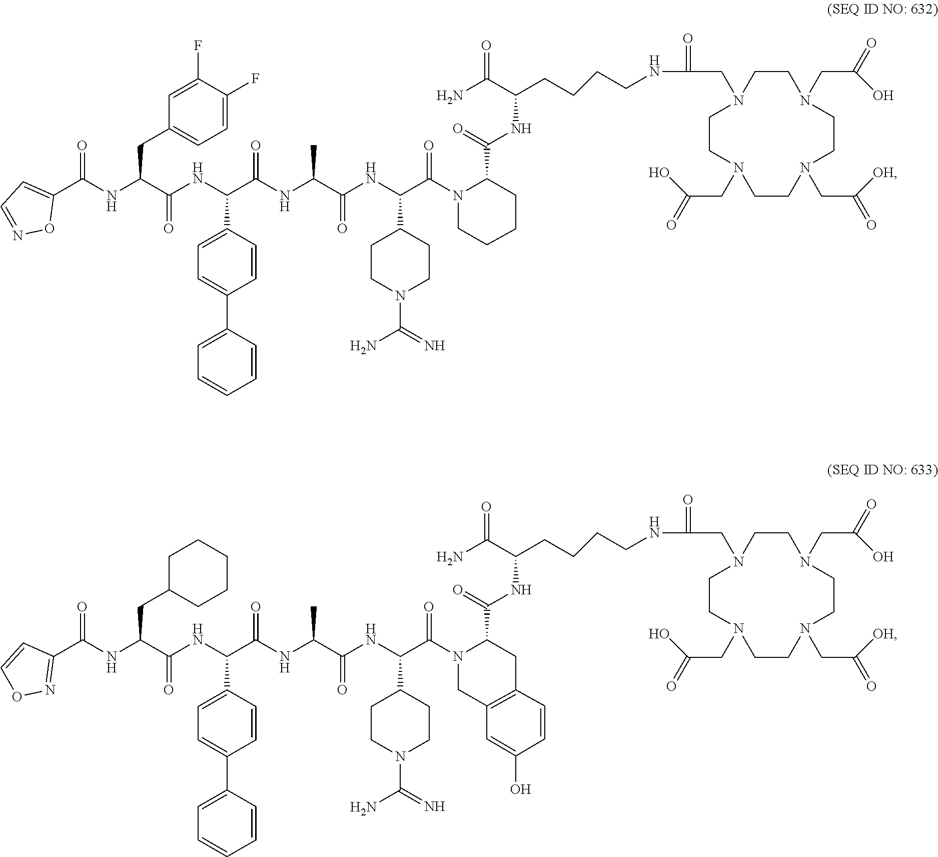 [SMILES: C[C@H](NC(=O)[C@@H](NC(=O)[C@H](Cc1ccc(F)c(F)c1)NC(=O)c1ccno1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](CC1CCCCC1)NC(=O)c1ccon1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(N)=O)C1CCN(C(=N)N)CC1] 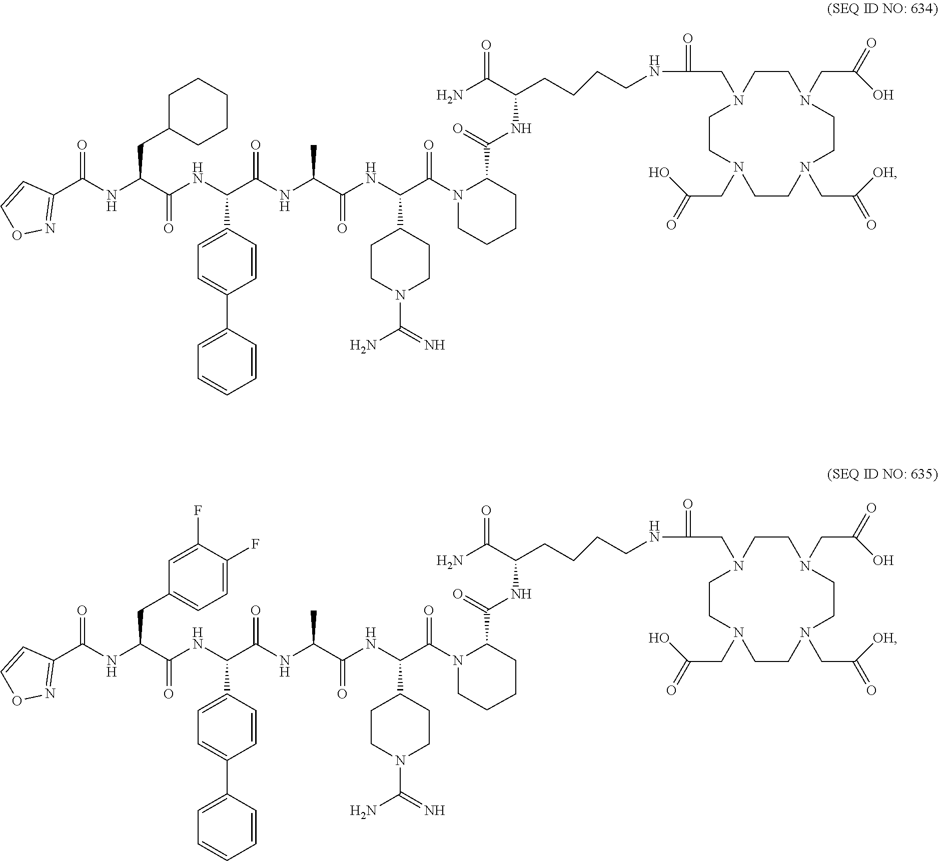 [SMILES: C[C@H](NC(=O)[C@@H](NC(=O)[C@H](CC1CCCCC1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](Cc1ccc(F)c(F)c1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(N)=O)C1CCN(C(=N)N)CC1] 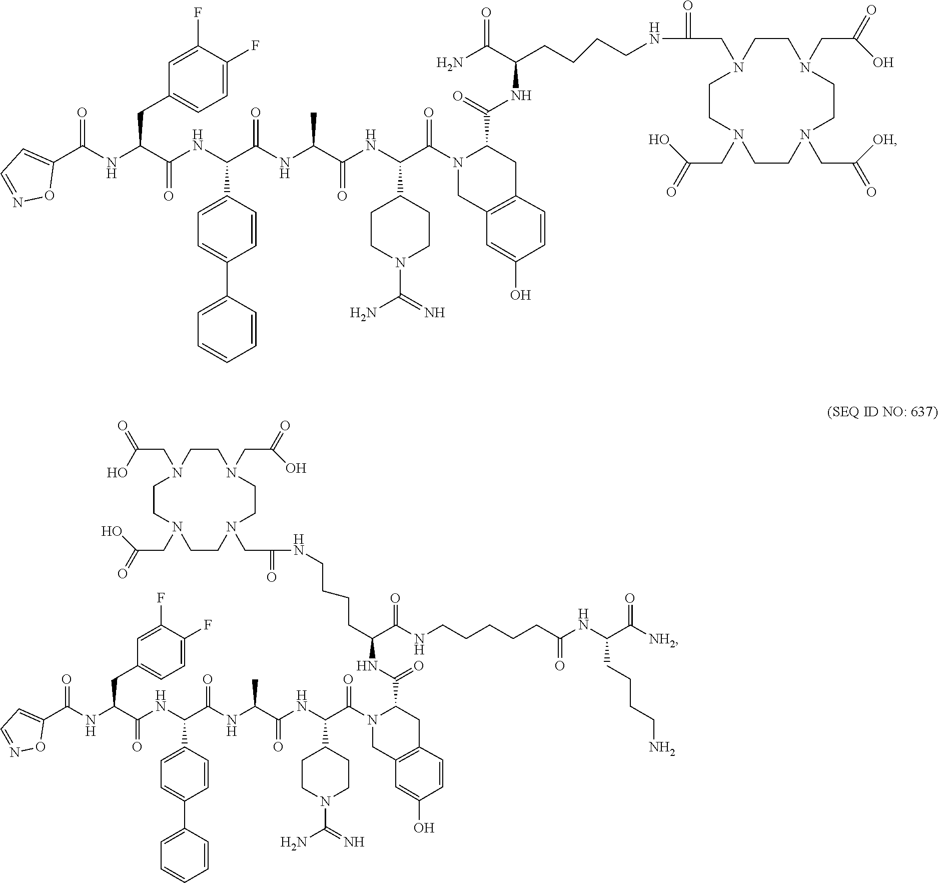 [SMILES: C[C@H](NC(=O)[C@@H](NC(=O)[C@H](Cc1ccc(F)c(F)c1)NC(=O)c1ccno1)c1ccc(-c2ccccc2)cc1)C(=O)N[C@H](C(=O)N1Cc2cc(O)ccc2C[C@H]1C(=O)N[C@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](Cc1ccc(F)c(F)c1)NC(=O)c1ccno1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1] 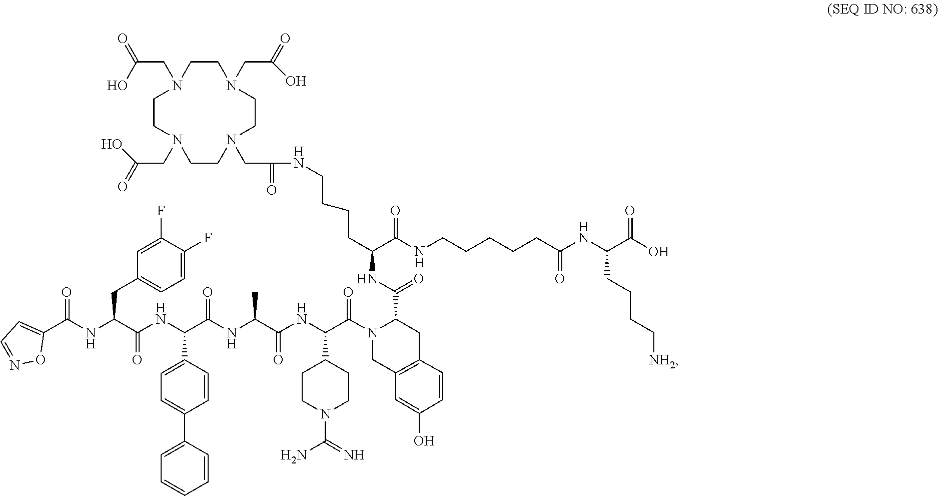 [SMILES: C[C@H](NC(=O)[C@@H](NC(=O)[C@H](Cc1ccc(F)c(F)c1)NC(=O)c1ccno1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(=O)O)C1CCN(C(=N)N)CC1] 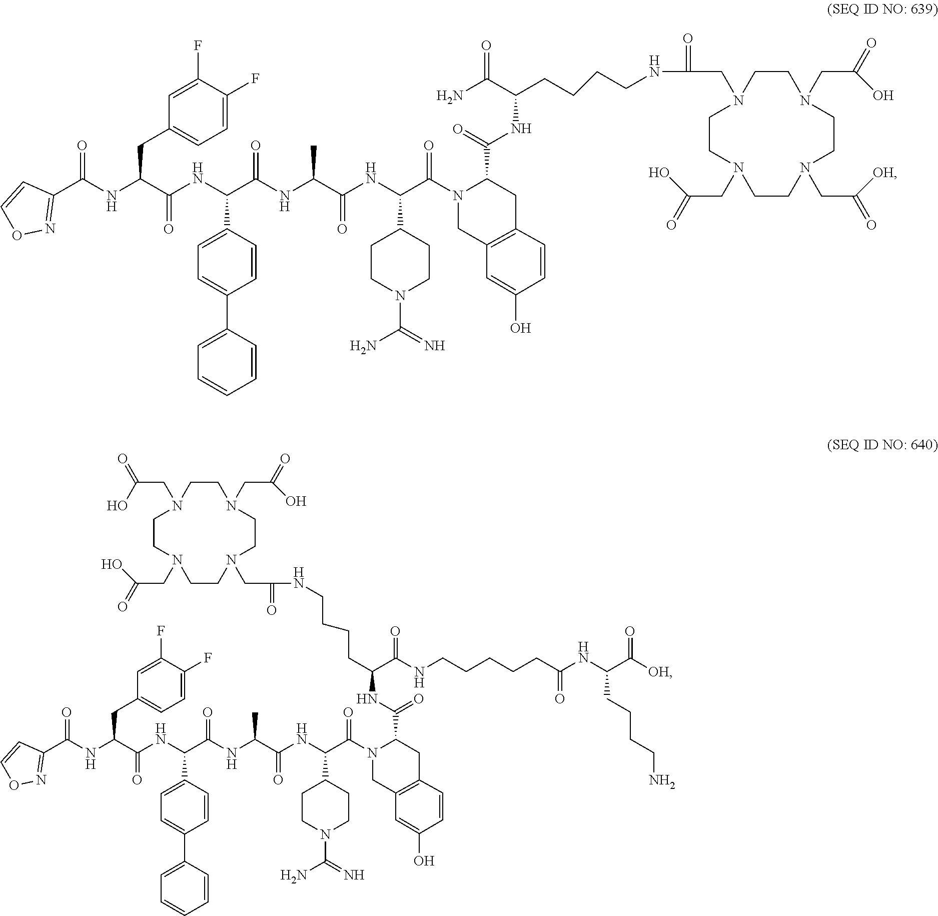 [SMILES: C[C@H](NC(=O)[C@@H](NC(=O)[C@H](Cc1ccc(F)c(F)c1)NC(=O)c1ccon1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(N)=O)C1CCN(C(=N)N)CC1 ; C[C@H](NC(=O)[C@@H](NC(=O)[C@H](Cc1ccc(F)c(F)c1)NC(=O)c1ccon1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(=O)O)C1CCN(C(=N)N)CC1] 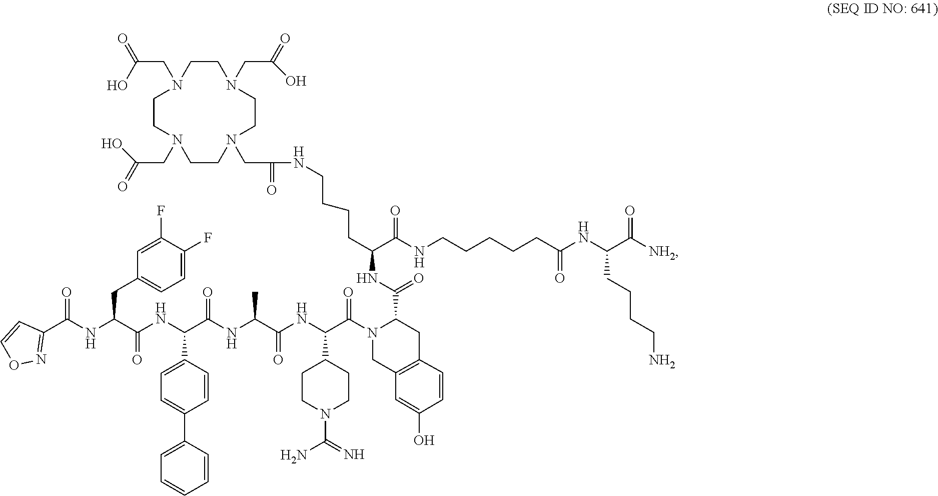 [SMILES: C[C@H](NC(=O)[C@@H](NC(=O)[C@H](Cc1ccc(F)c(F)c1)NC(=O)c1ccon1)c1ccc(-c2ccccc2)cc1)C(=O)N[C@H](C(=O)N1Cc2cc(O)ccc2C[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1] 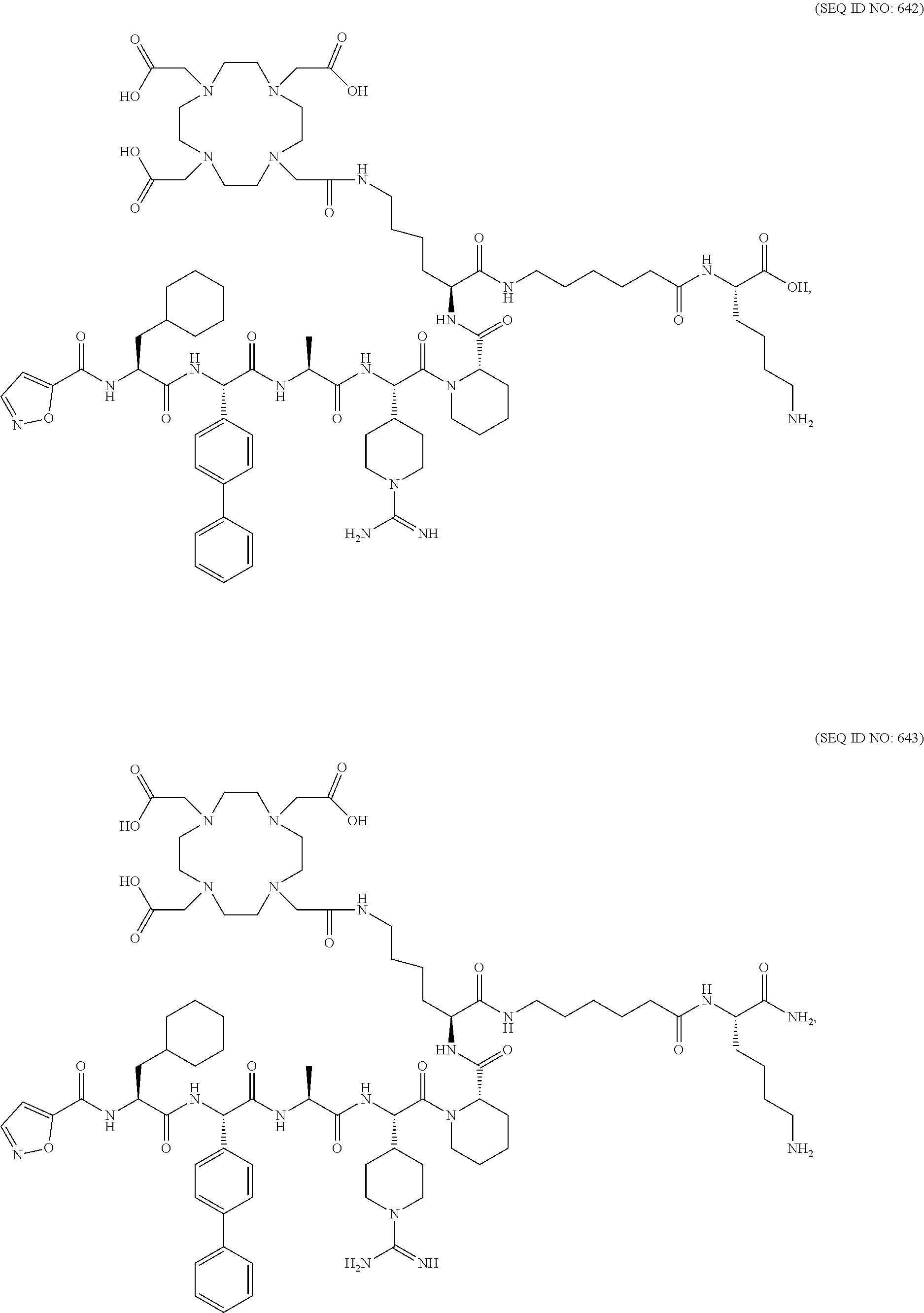 [SMILES: C[C@H](NC(=O)[C@@H](NC(=O)[C@H](CC1CCCCC1)NC(=O)c1ccno1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(=O)O)C1CCN(C(=N)N)CC1 ; C[C@H](NC(=O)[C@@H](NC(=O)[C@H](CC1CCCCC1)NC(=O)c1ccno1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1] 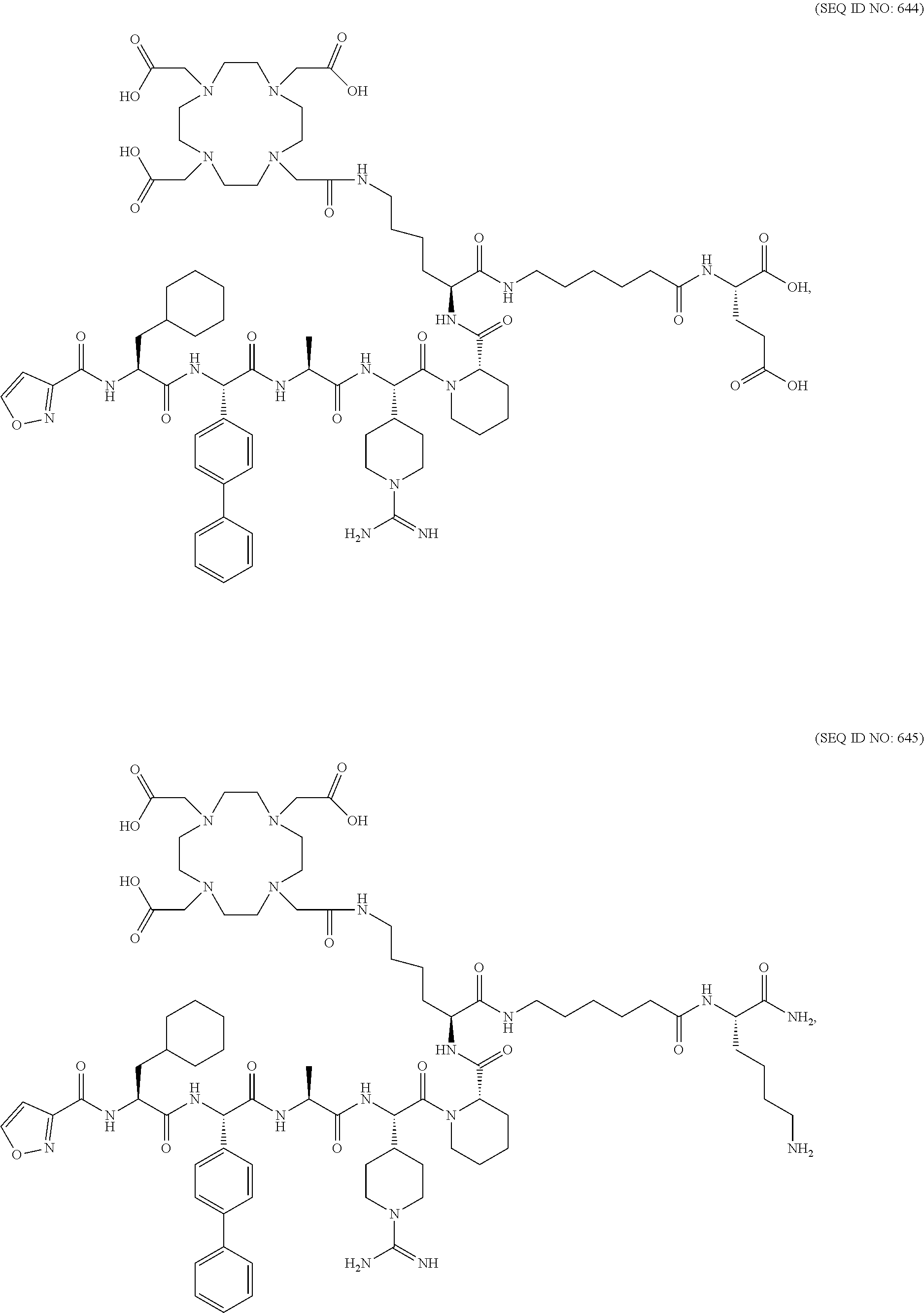 [SMILES: C[C@H](NC(=O)[C@@H](NC(=O)[C@H](CC1CCCCC1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCC(=O)O)C(=O)O)C1CCN(C(=N)N)CC1 ; C[C@H](NC(=O)[C@@H](NC(=O)[C@H](CC1CCCCC1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(N)=O)C1CCN(C(=N)N)CC1] 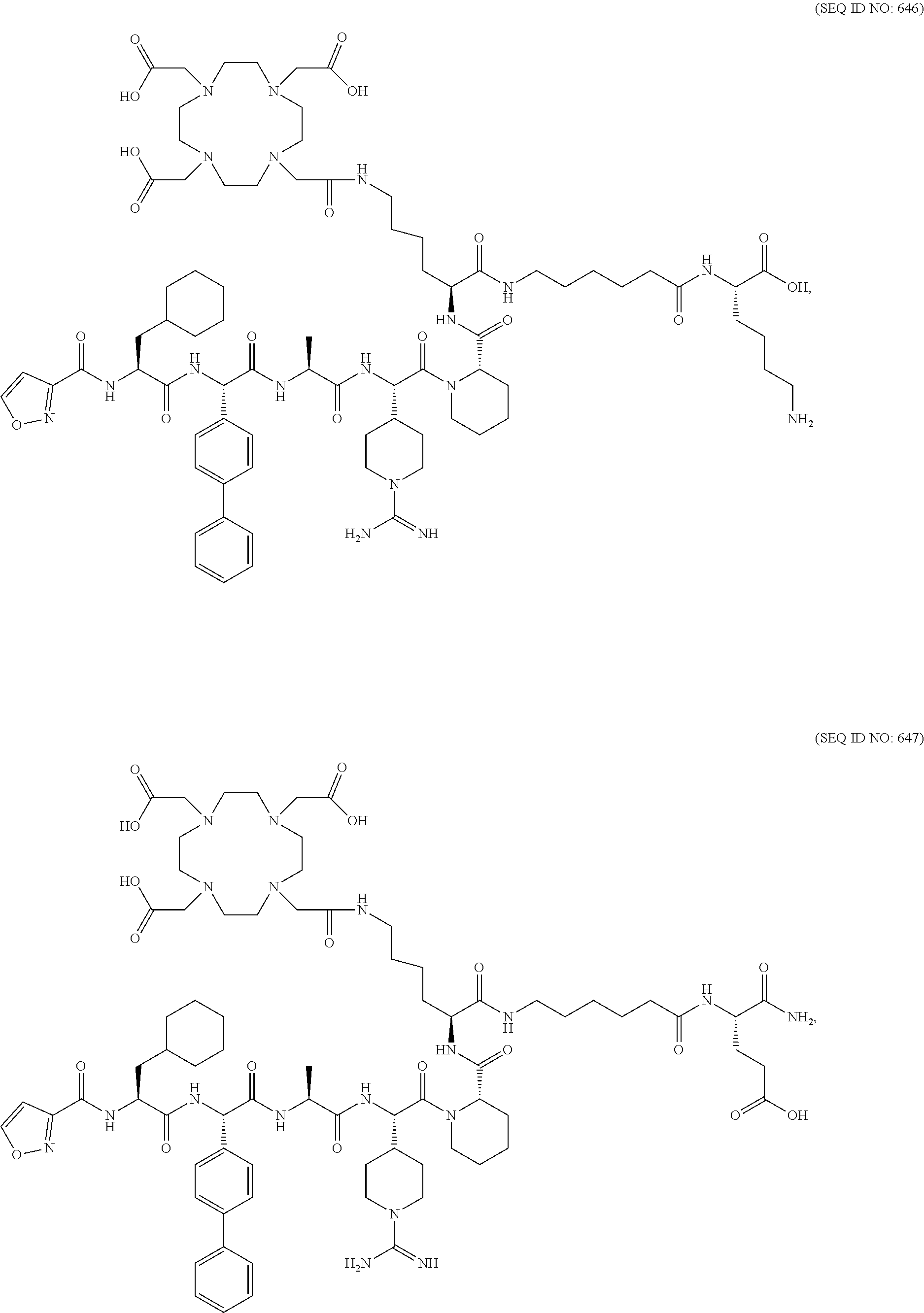 [SMILES: C[C@H](NC(=O)[C@@H](NC(=O)[C@H](CC1CCCCC1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCCCN)C(=O)O)C1CCN(C(=N)N)CC1 ; C[C@H](NC(=O)[C@@H](NC(=O)[C@H](CC1CCCCC1)NC(=O)c1ccon1)c1ccc(-c2ccccc2)cc1)C(=O)N[C@H](C(=O)N1CCCC[C@H]1C(=O)N[C@@H](CCCCNC(=O)CN1CCN(CC(=O)O)CCN(CC(=O)O)CCN(CC(=O)O)CC1)C(=O)NCCCCCC(=O)N[C@@H](CCC(=O)O)C(N)=O)C1CCN(C(=N)N)CC1] 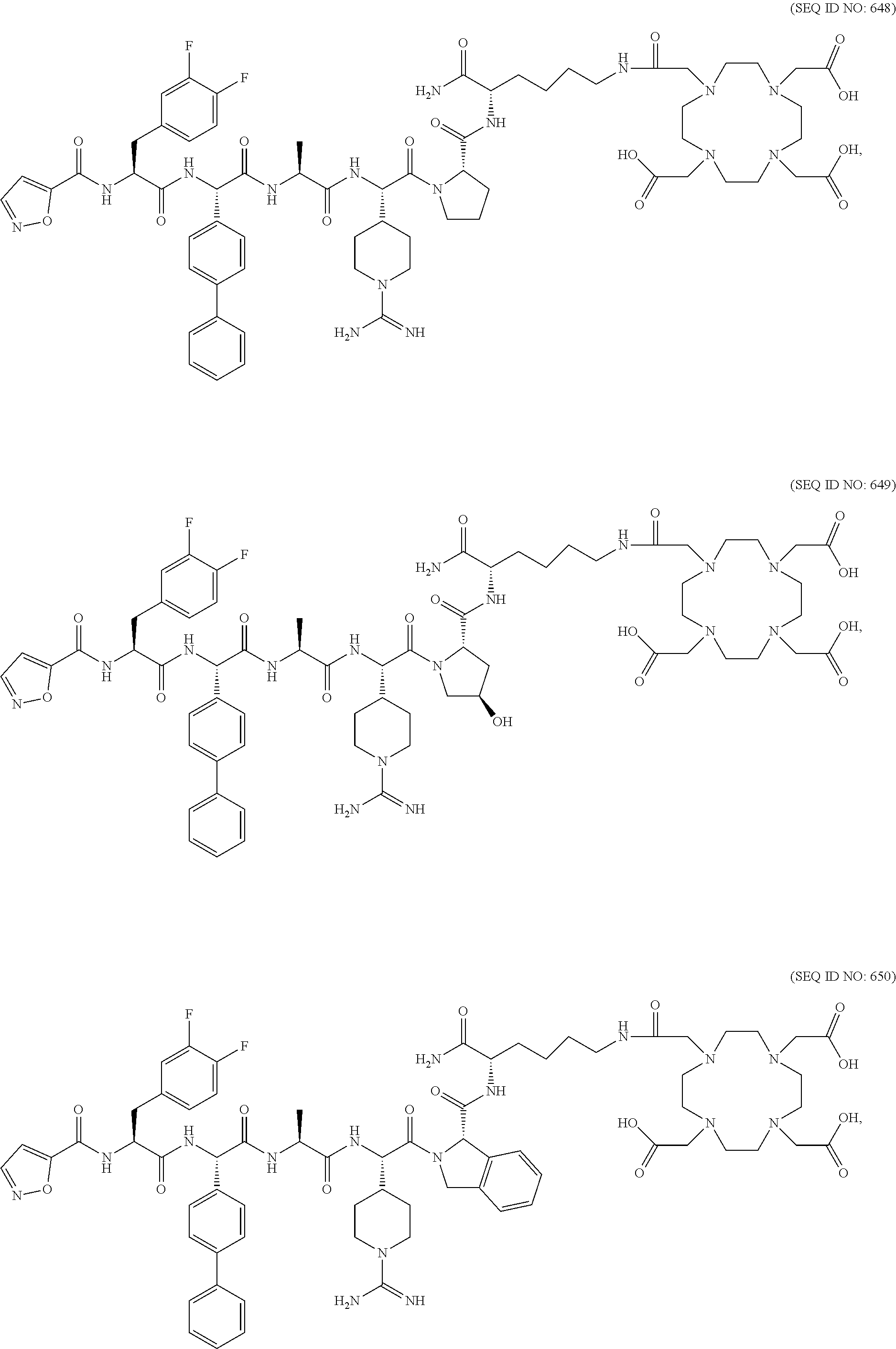 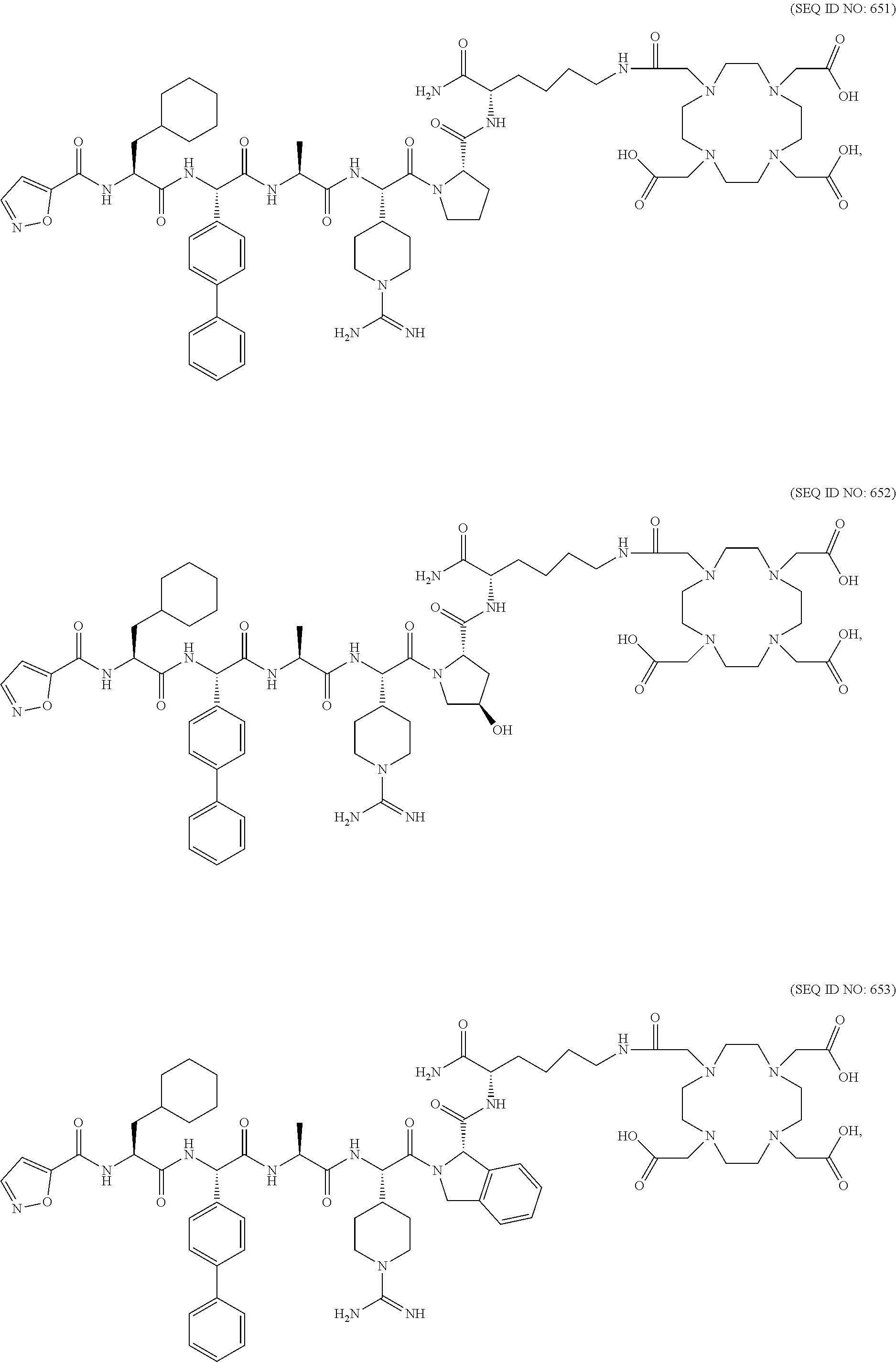 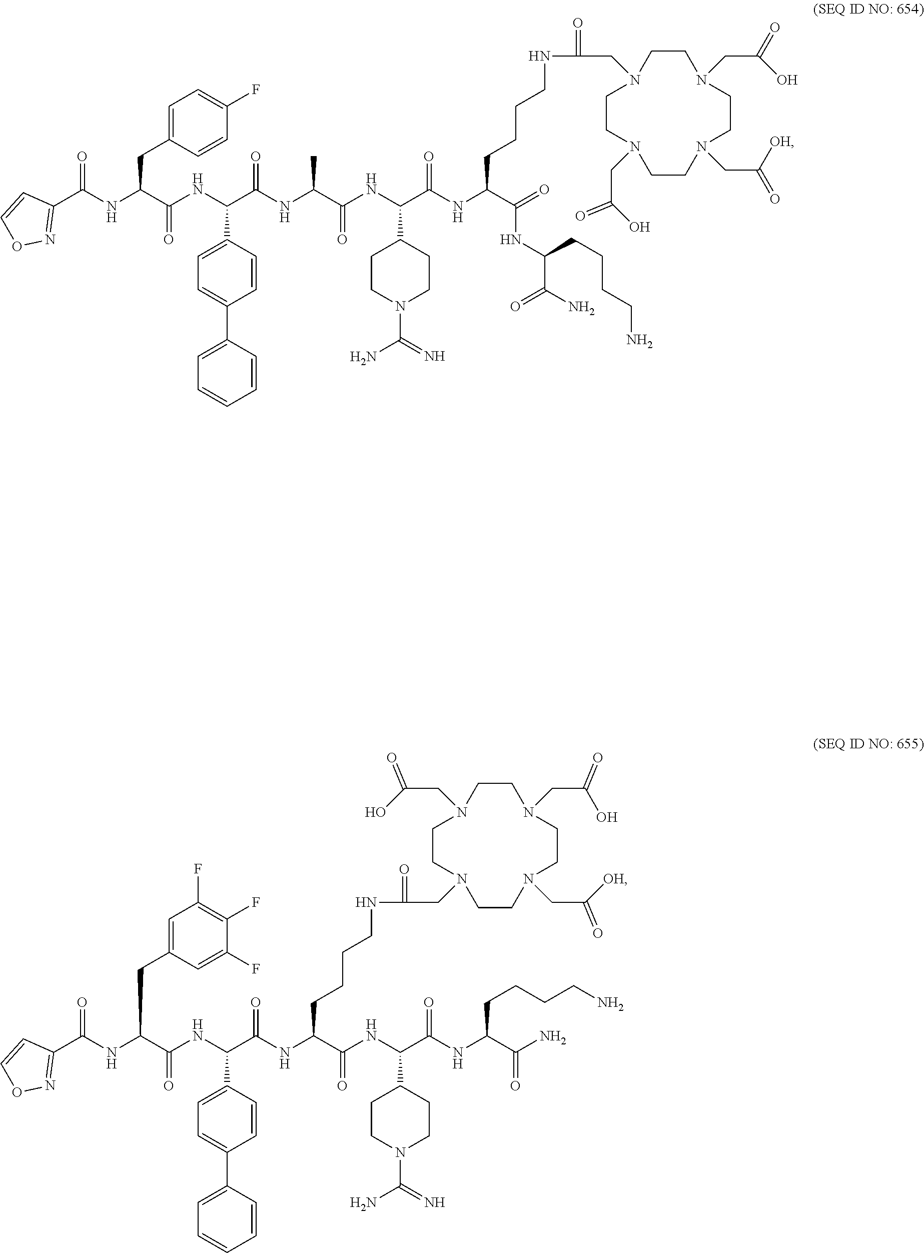 [SMILES: C[C@H](NC(=O)[C@@H](NC(=O)[C@H](Cc1ccc(F)cc1)NC(=O)c1ccon1)c1ccc(-c2ccccc2)cc1)C(=O)N[C@H](C(=O)N[C@@H](CCCCNC(=O)CN1CCN(CC(=O)O)CCN(CC(=O)O)CCN(CC(=O)O)CC1)C(=O)N[C@@H](CCCCN)C(N)=O)C1CCN(C(=N)N)CC1 ; N=C(N)N1CCC([C@H](NC(=O)[C@H](CCCCNC(=O)CN2CCN(CC(=O)O)CCN(CC(=O)O)CCN(CC(=O)O)CC2)NC(=O)[C@@H](NC(=O)[C@H](Cc2cc(F)c(F)c(F)c2)NC(=O)c2ccon2)c2ccc(-c3ccccc3)cc2)C(=O)N[C@@H](CCCCN)C(N)=O)CC1] 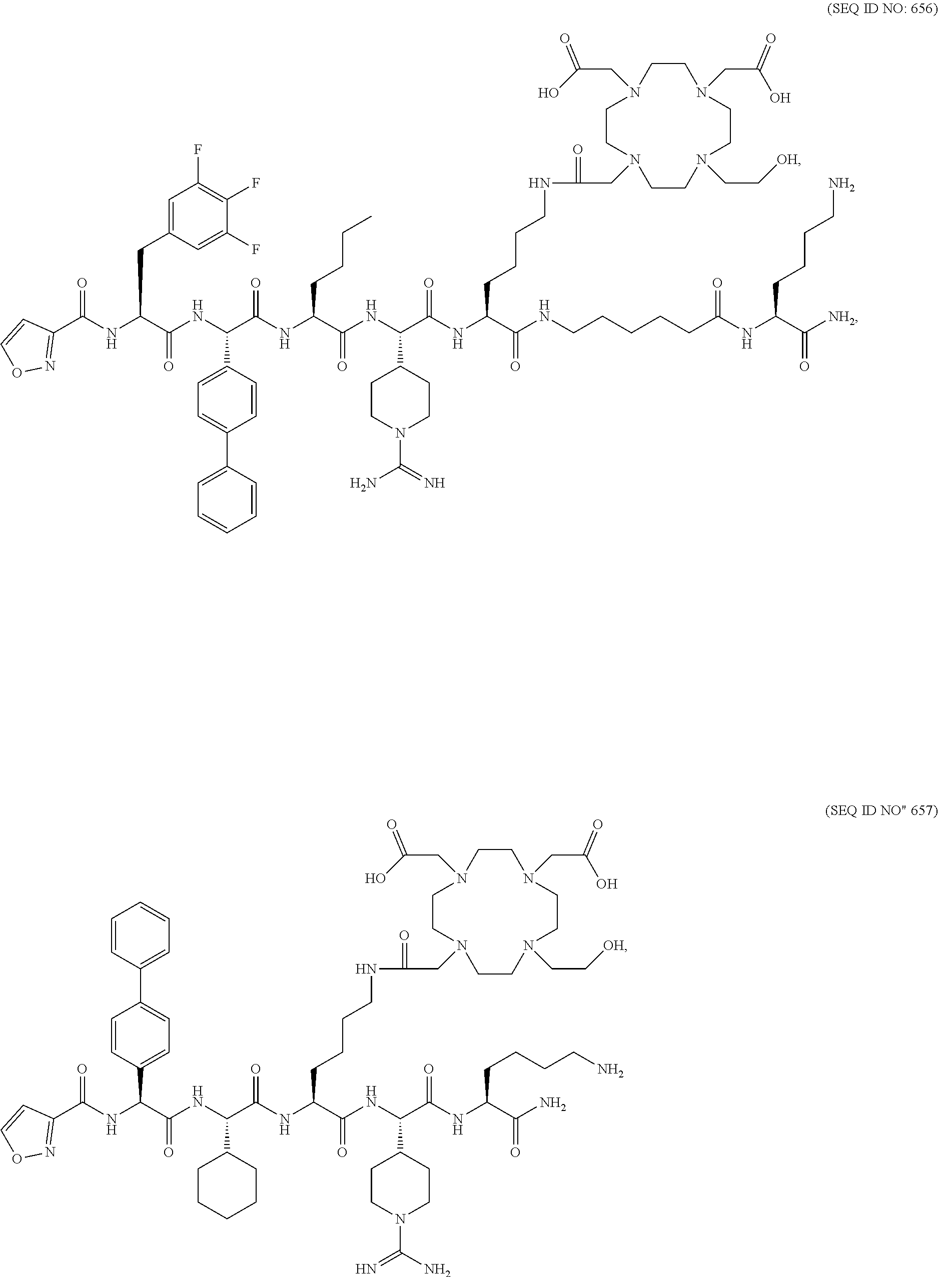 [SMILES: CCCC[C@H](NC(=O)[C@@H](NC(=O)[C@H](Cc1cc(F)c(F)c(F)c1)NC(=O)c1ccon1)c1ccc(-c2ccccc2)cc1)C(=O)N[C@H](C(=O)N[C@@H](CCCCNC(=O)CN1CCN(CCO)CCN(CC(=O)O)CCN(CC(=O)O)CC1)C(=O)NCCCCCC(=O)N[C@@H](CCCCN)C(N)=O)C1CCN(C(=N)N)CC1 ; N=C(N)N1CCC([C@H](NC(=O)[C@H](CCCCNC(=O)CN2CCN(CCO)CCN(CC(=O)O)CCN(CC(=O)O)CC2)NC(=O)[C@@H](NC(=O)[C@@H](NC(=O)c2ccon2)c2ccc(-c3ccccc3)cc2)C2CCCCC2)C(=O)N[C@@H](CCCCN)C(N)=O)CC1] 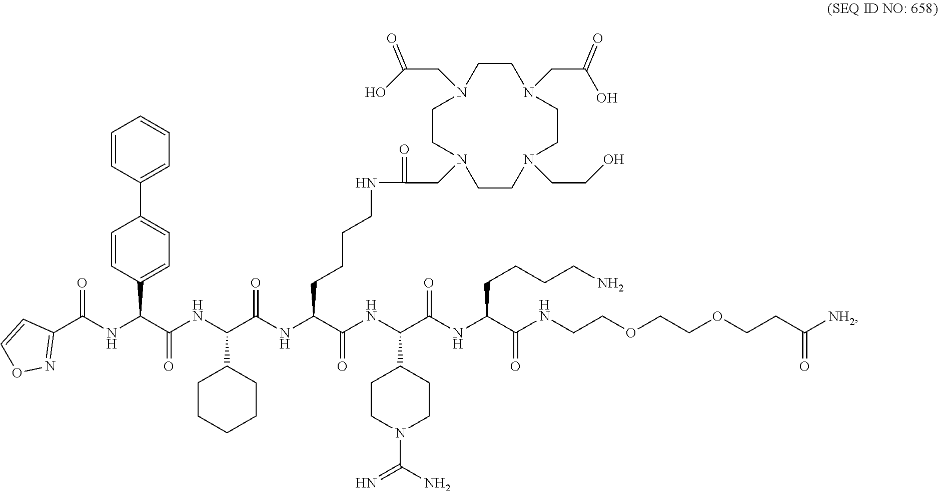 [SMILES: N=C(N)N1CCC([C@H](NC(=O)[C@H](CCCCNC(=O)CN2CCN(CCO)CCN(CC(=O)O)CCN(CC(=O)O)CC2)NC(=O)[C@@H](NC(=O)[C@@H](NC(=O)c2ccon2)c2ccc(-c3ccccc3)cc2)C2CCCCC2)C(=O)N[C@@H](CCCCN)C(=O)NCCOCCOCCC(N)=O)CC1] 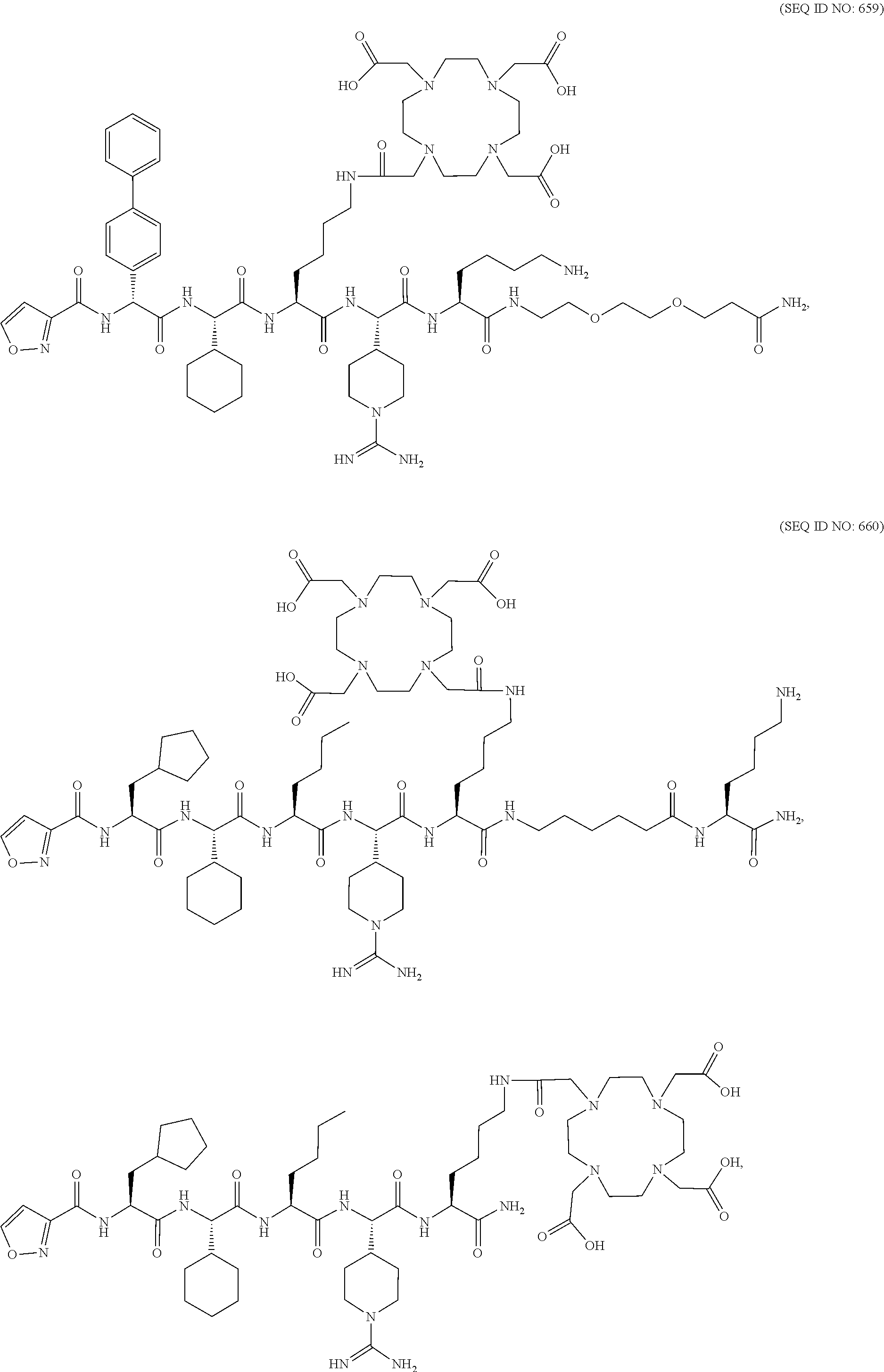 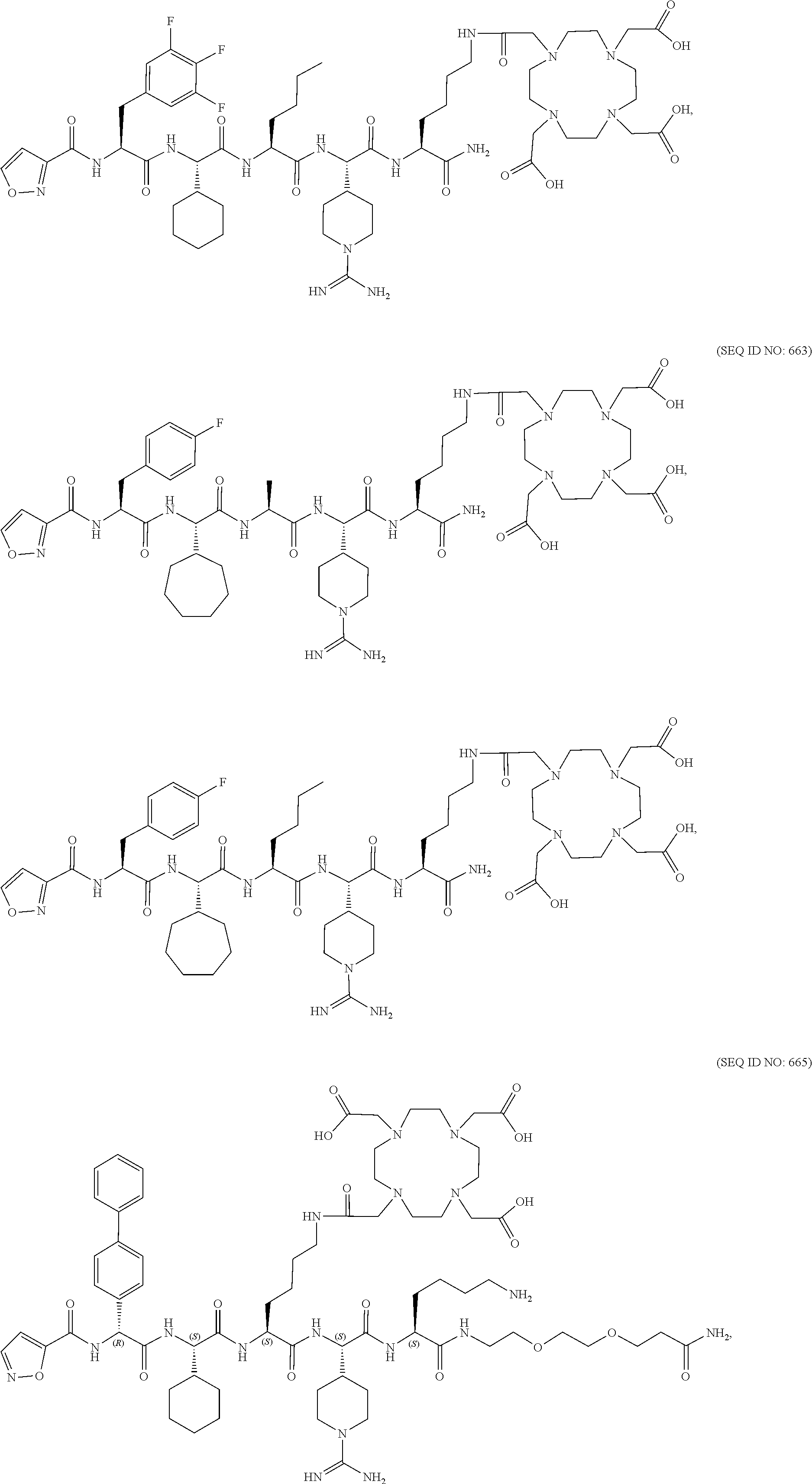 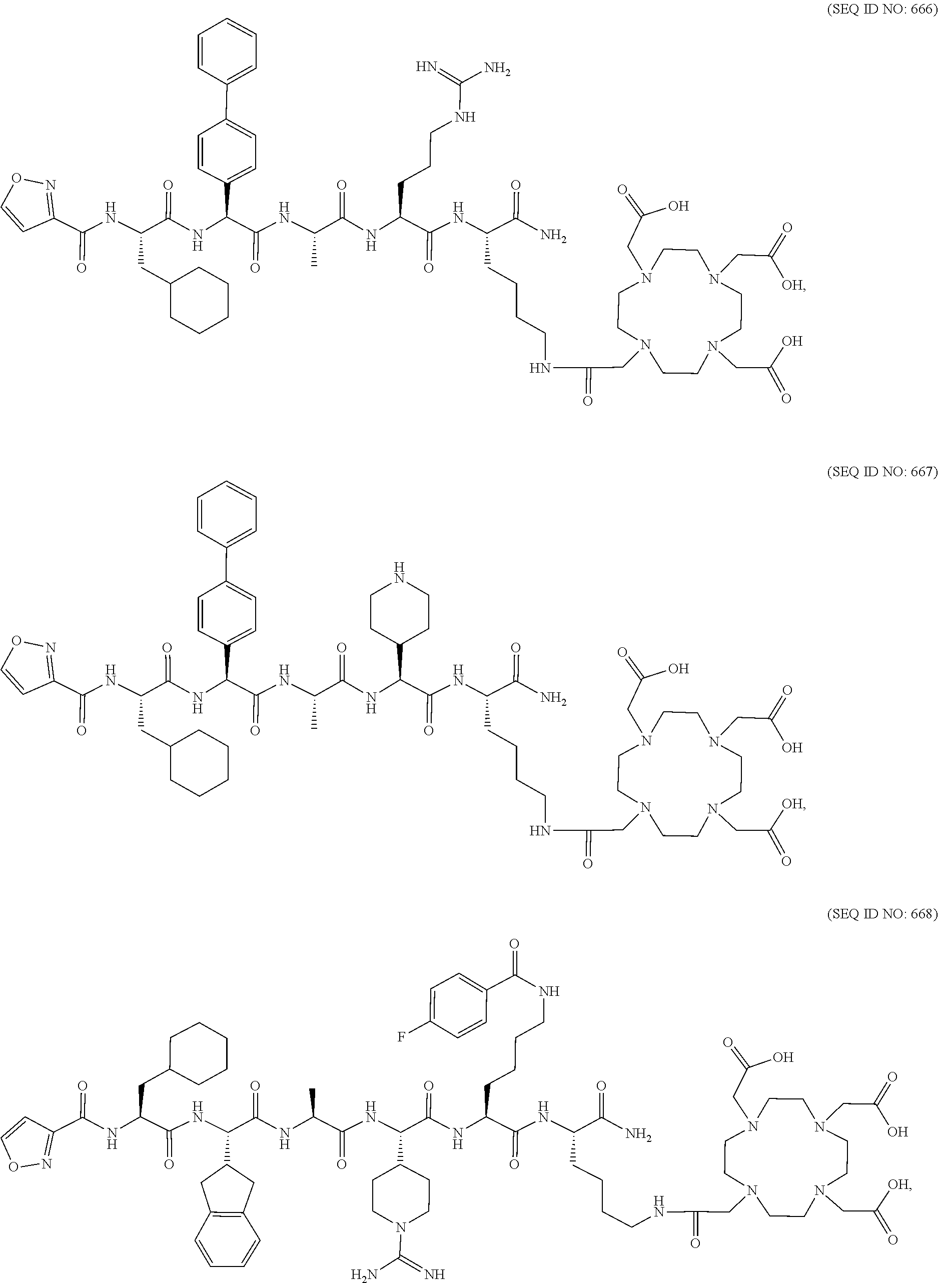 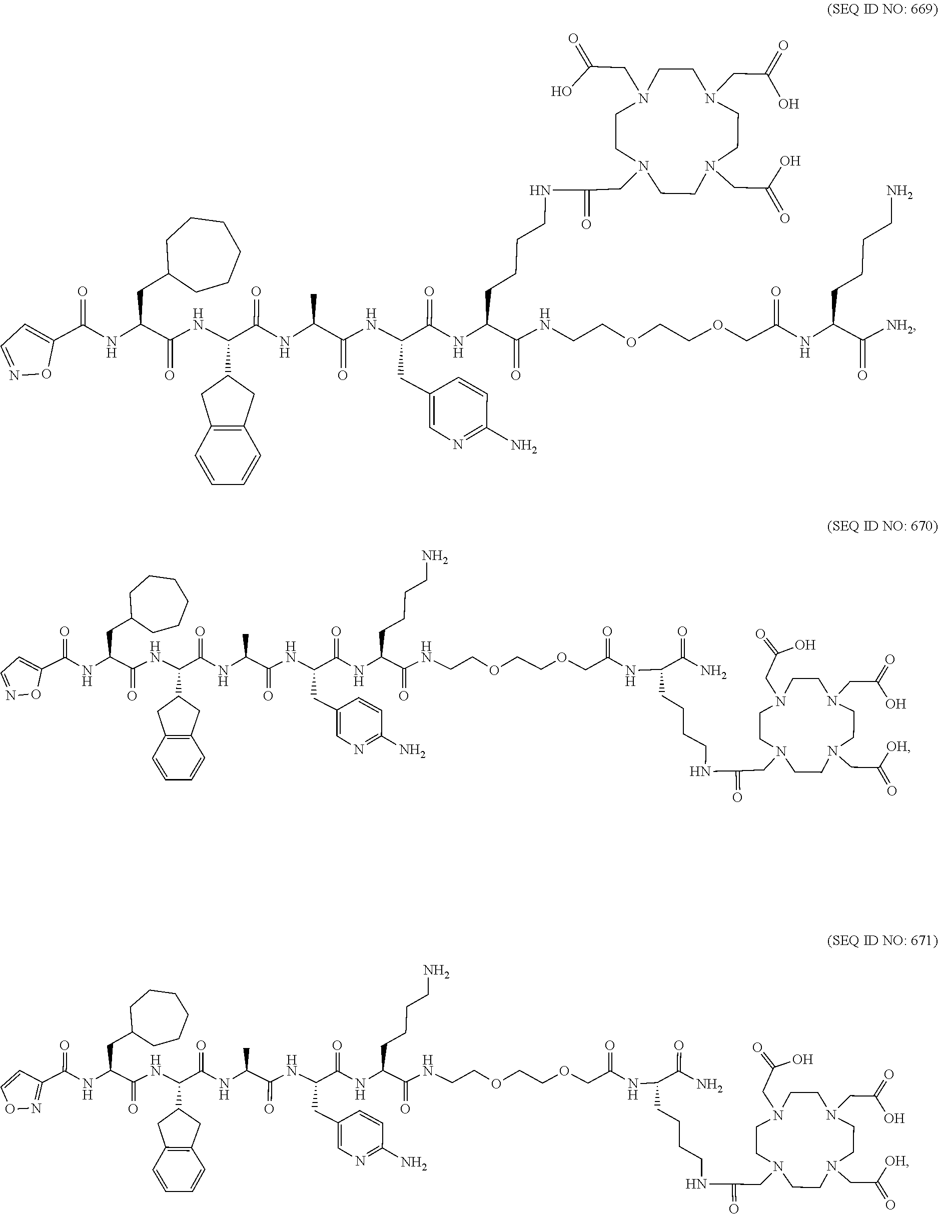 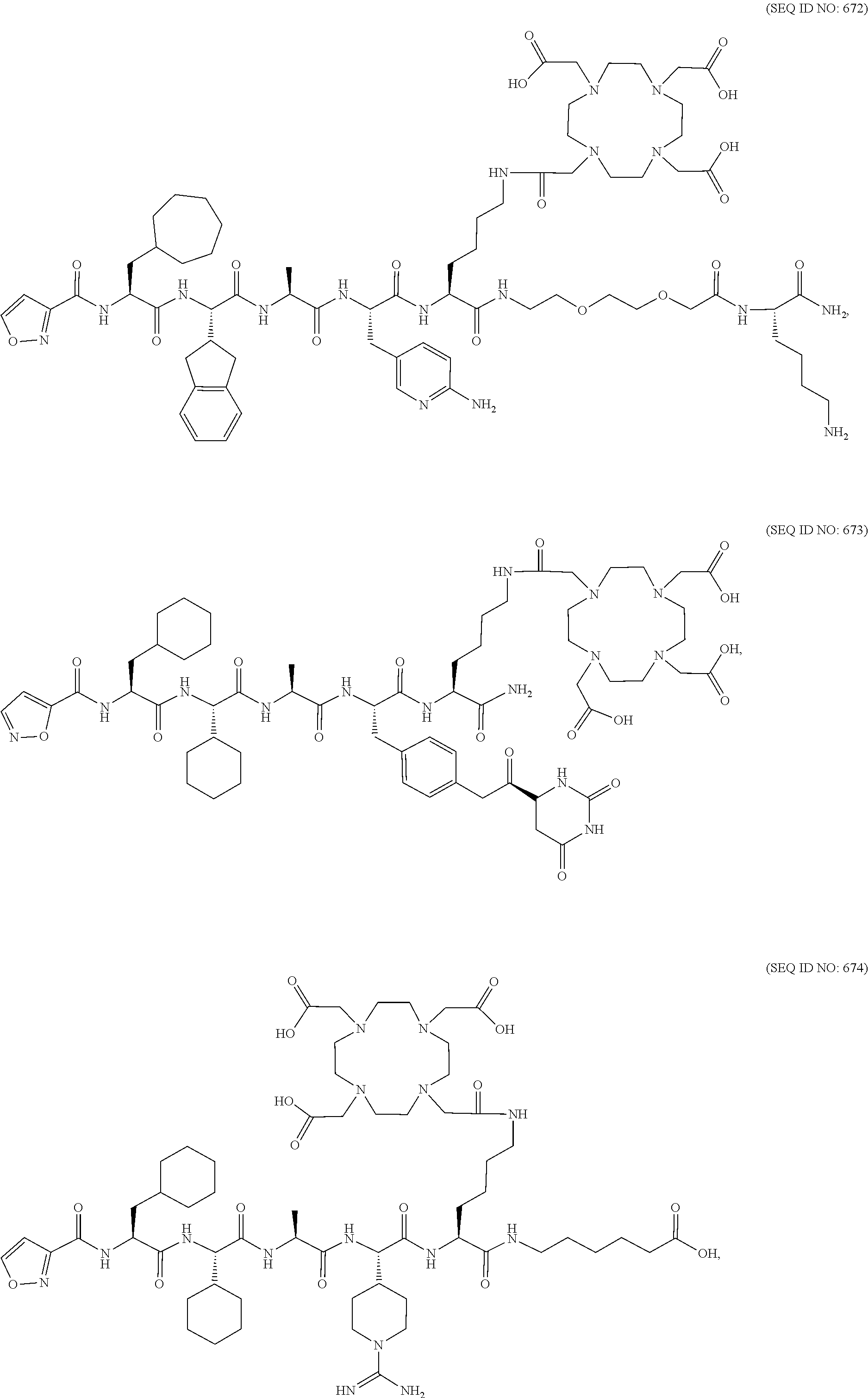 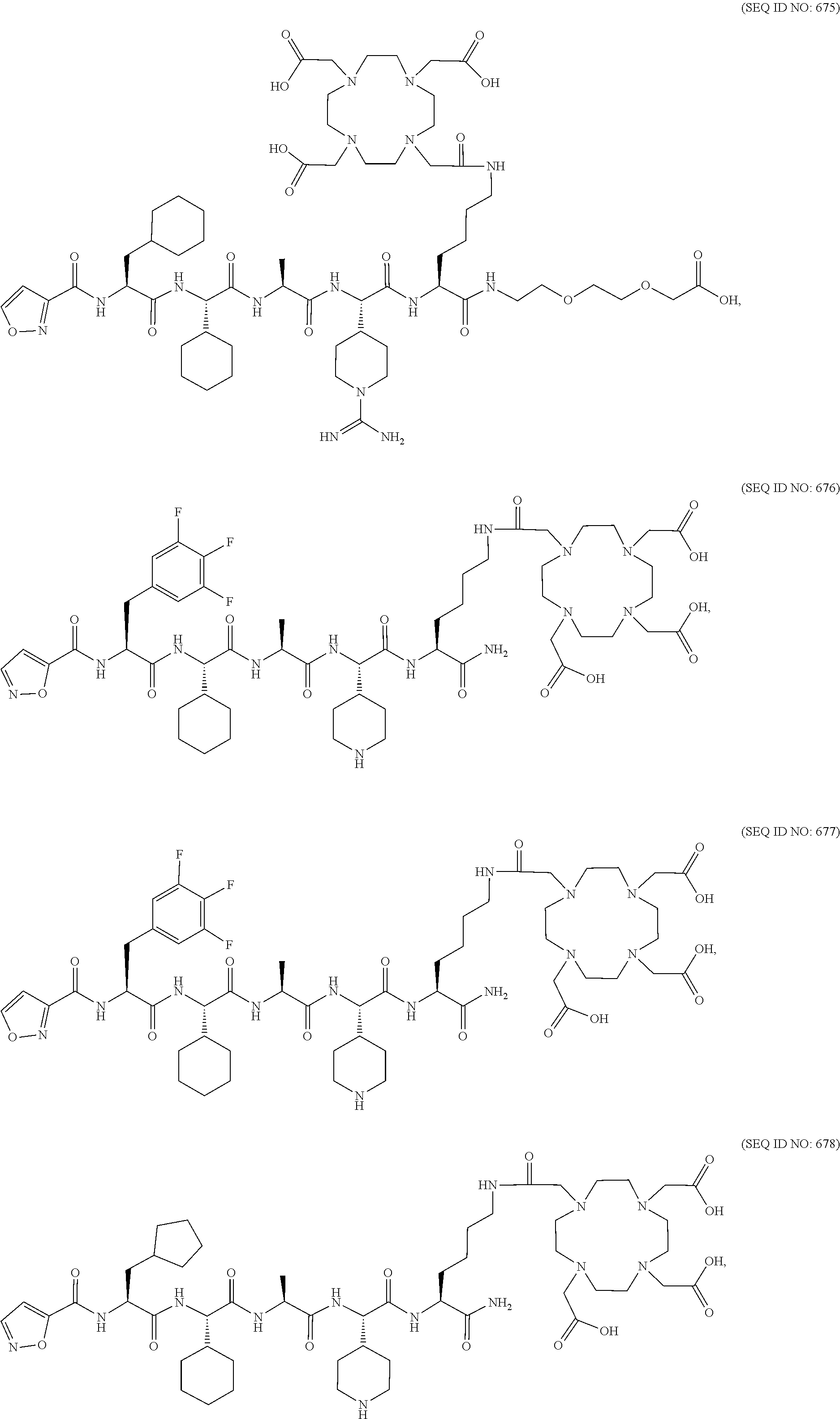 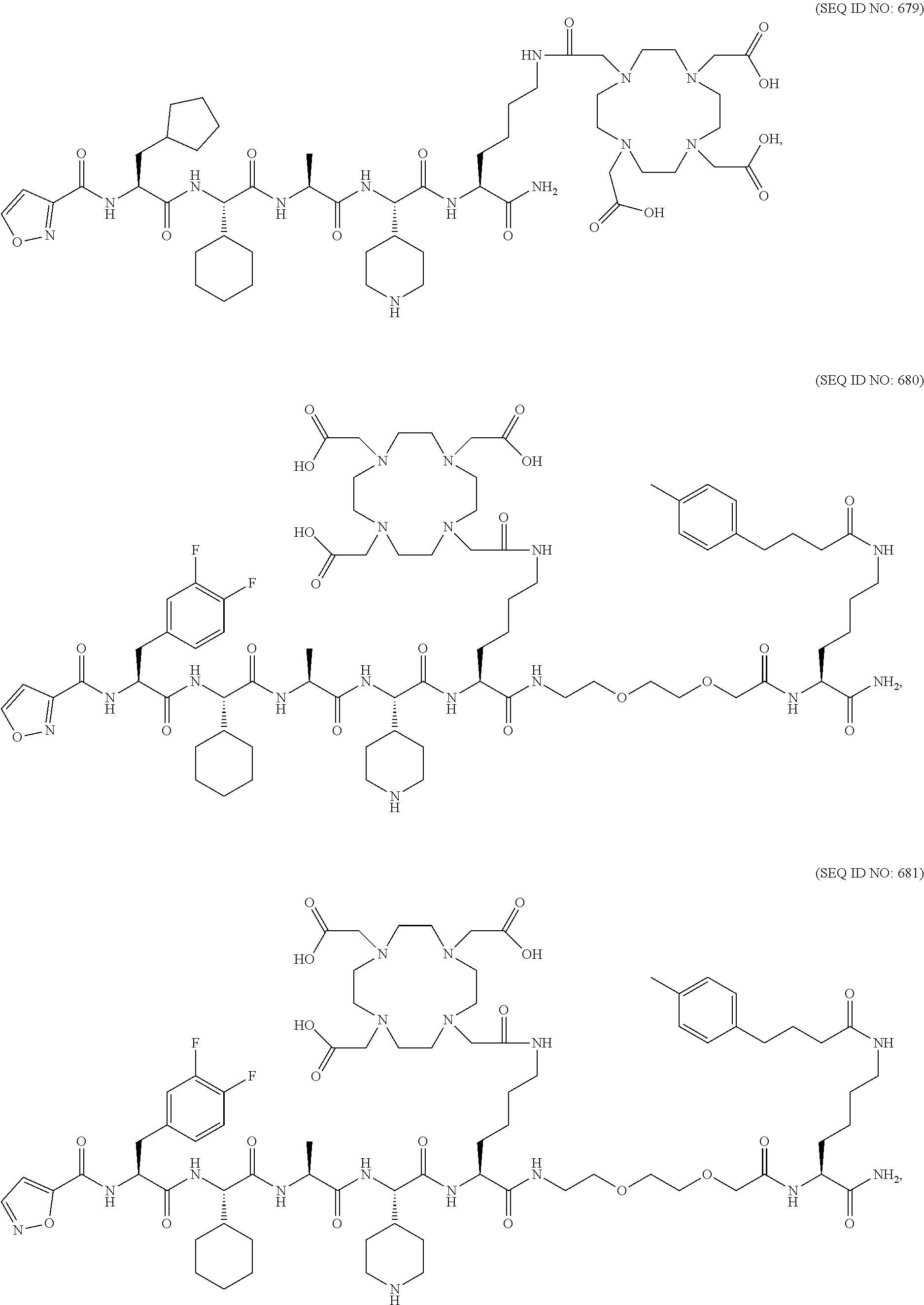 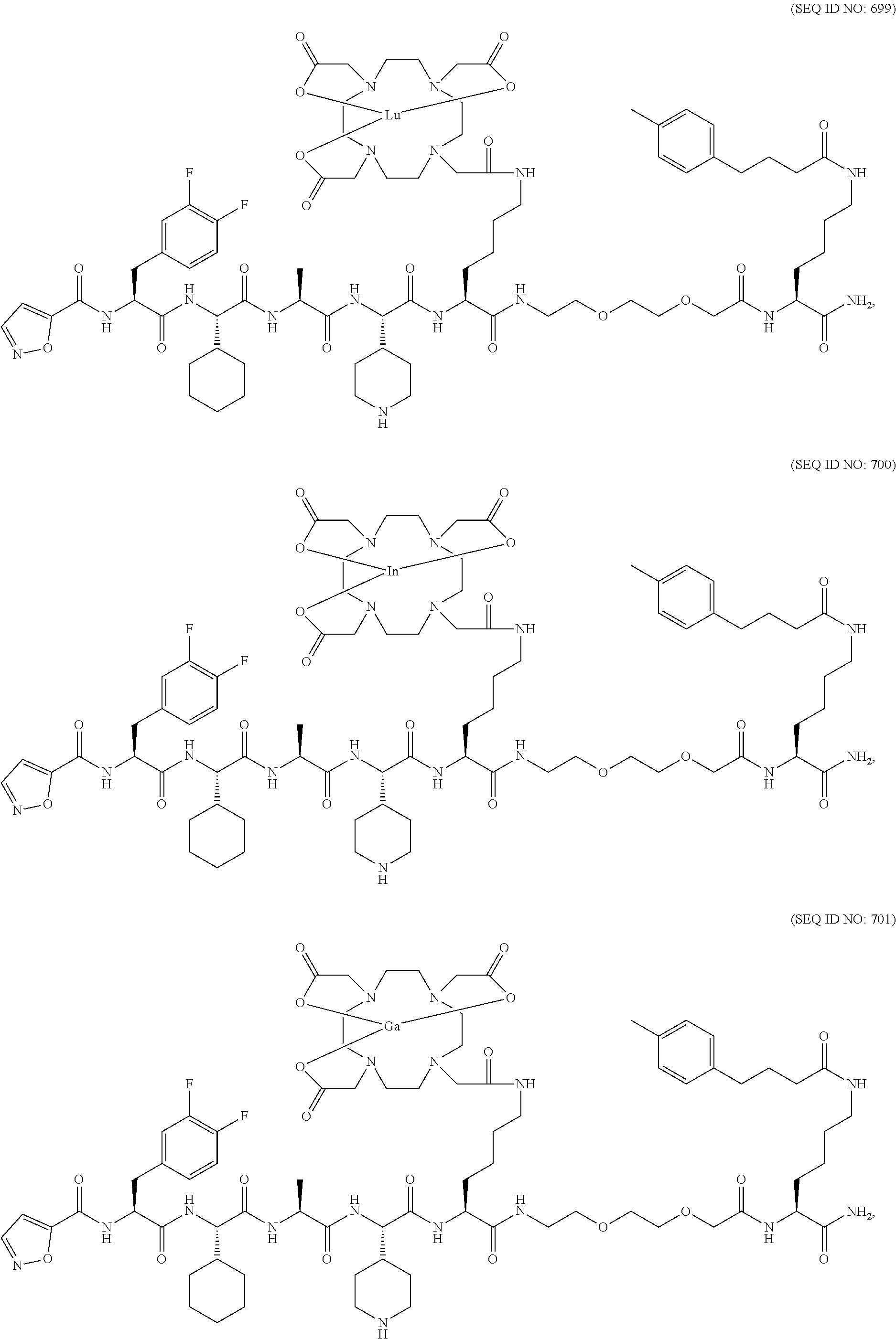 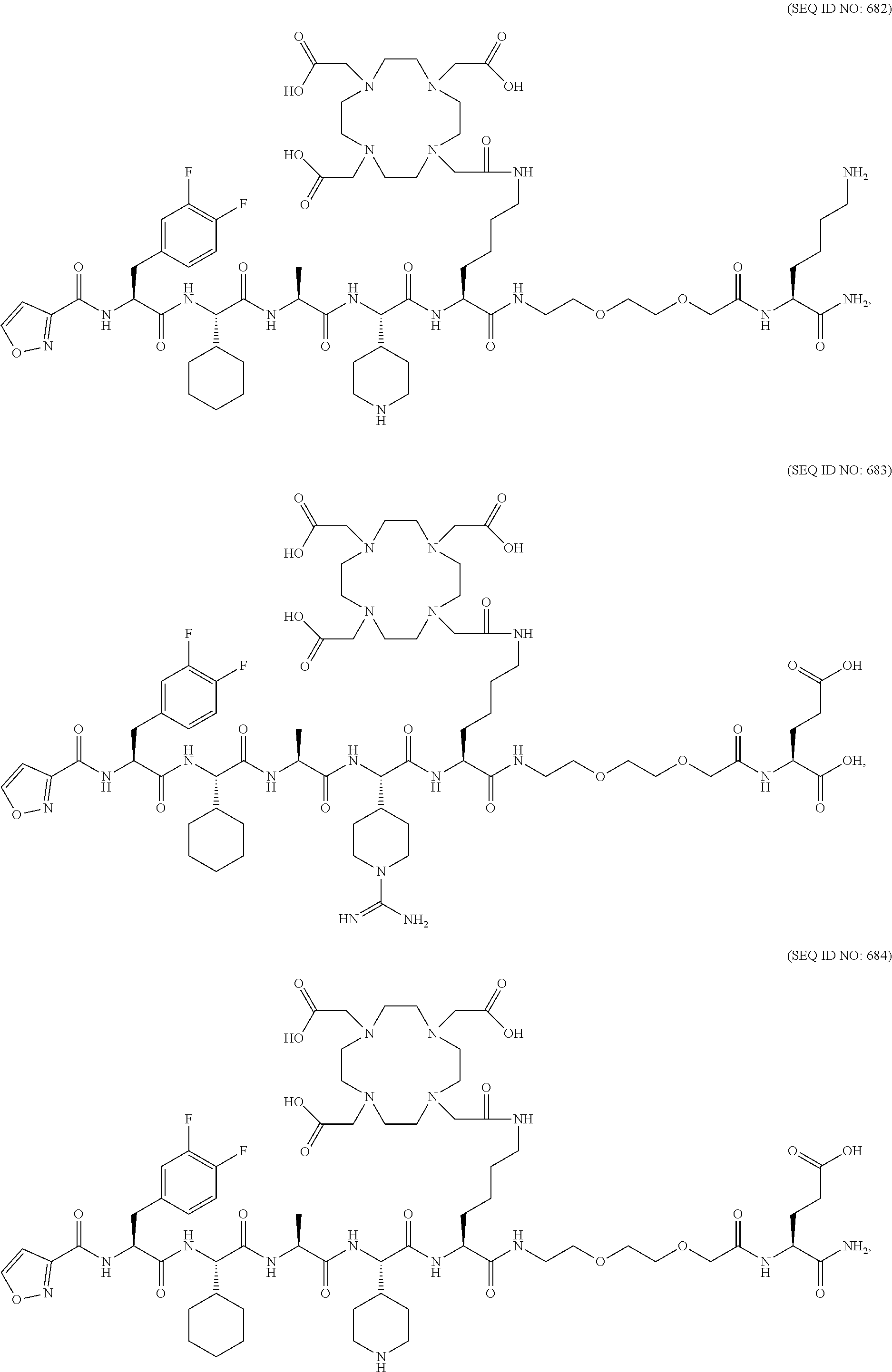 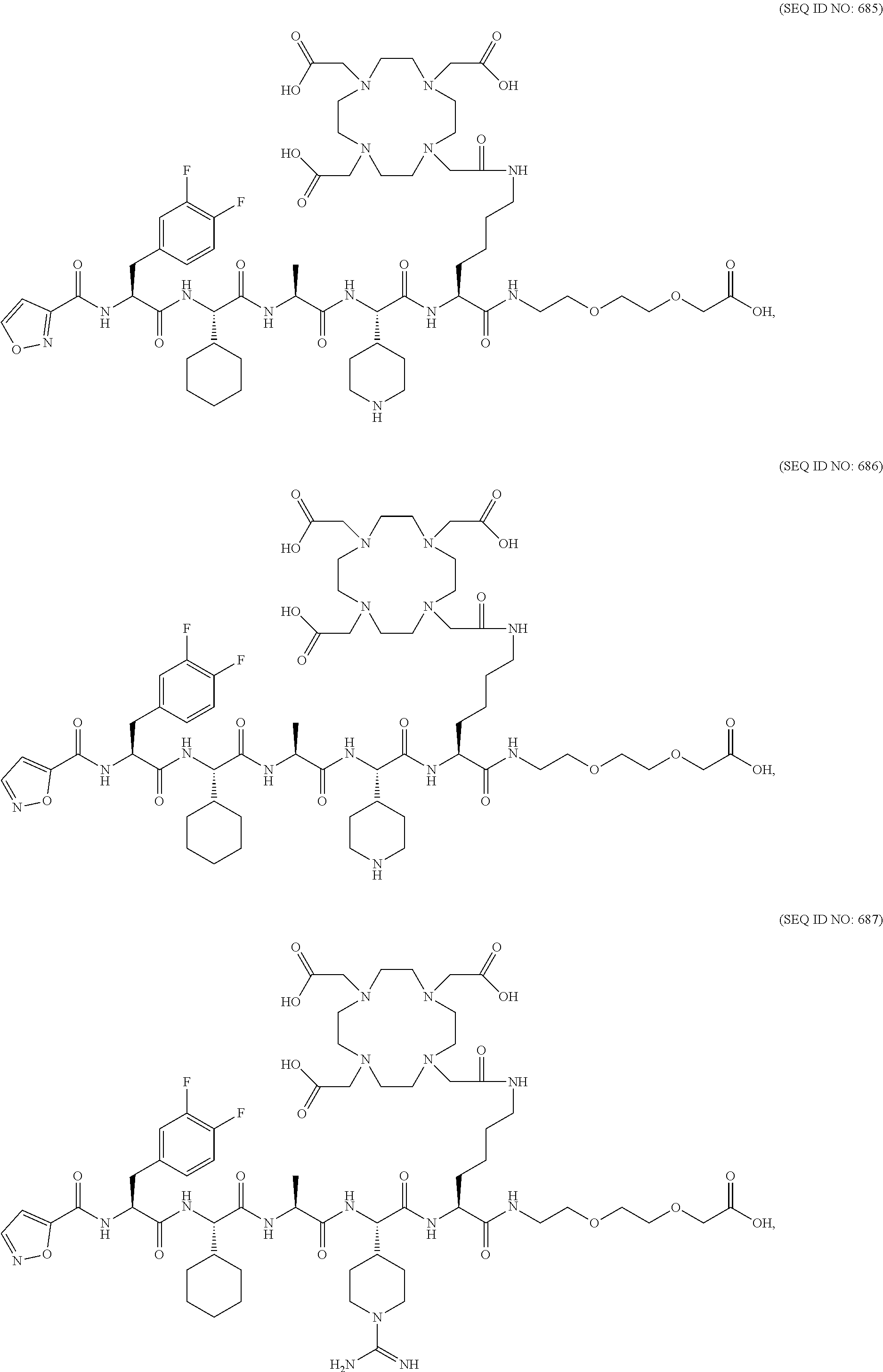 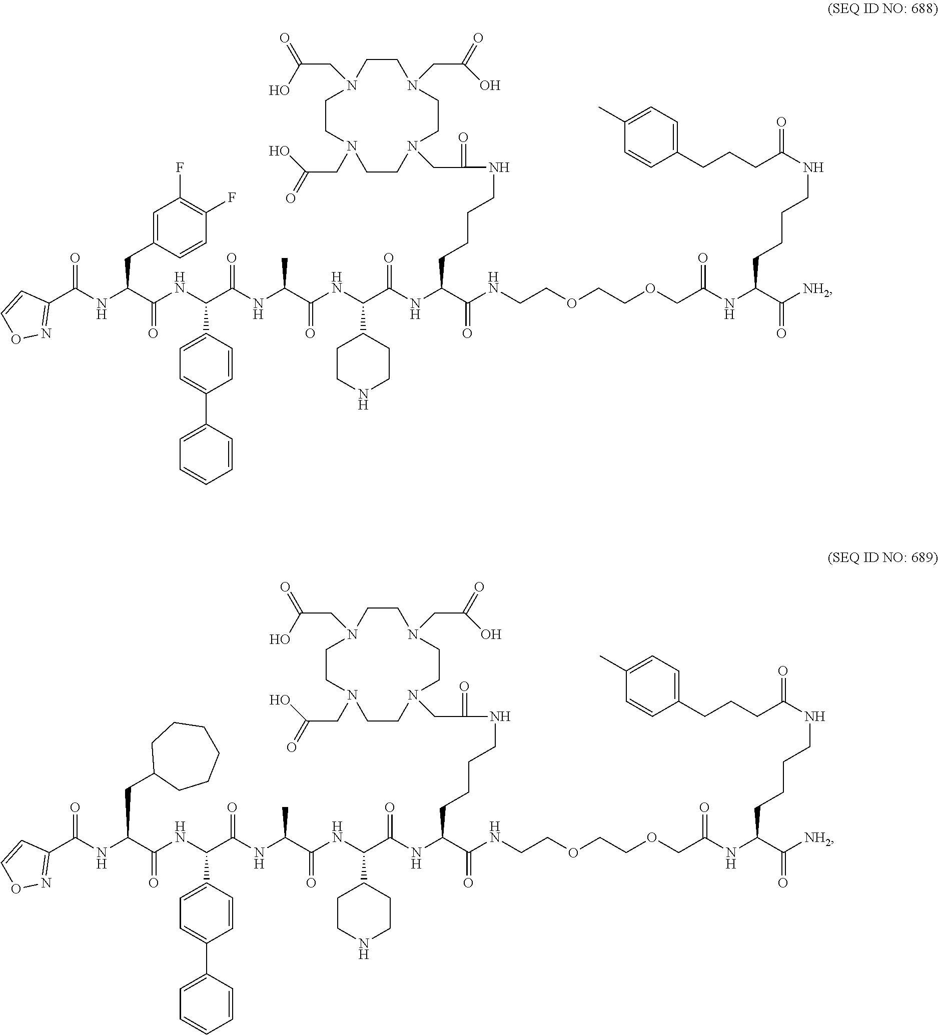 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2ccon2)c2ccc(-c3ccccc3)cc2)C2CCNCC2)C(N)=O)cc1 ; Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](CC2CCCCCC2)NC(=O)c2ccon2)c2ccc(-c3ccccc3)cc2)C2CCNCC2)C(N)=O)cc1] 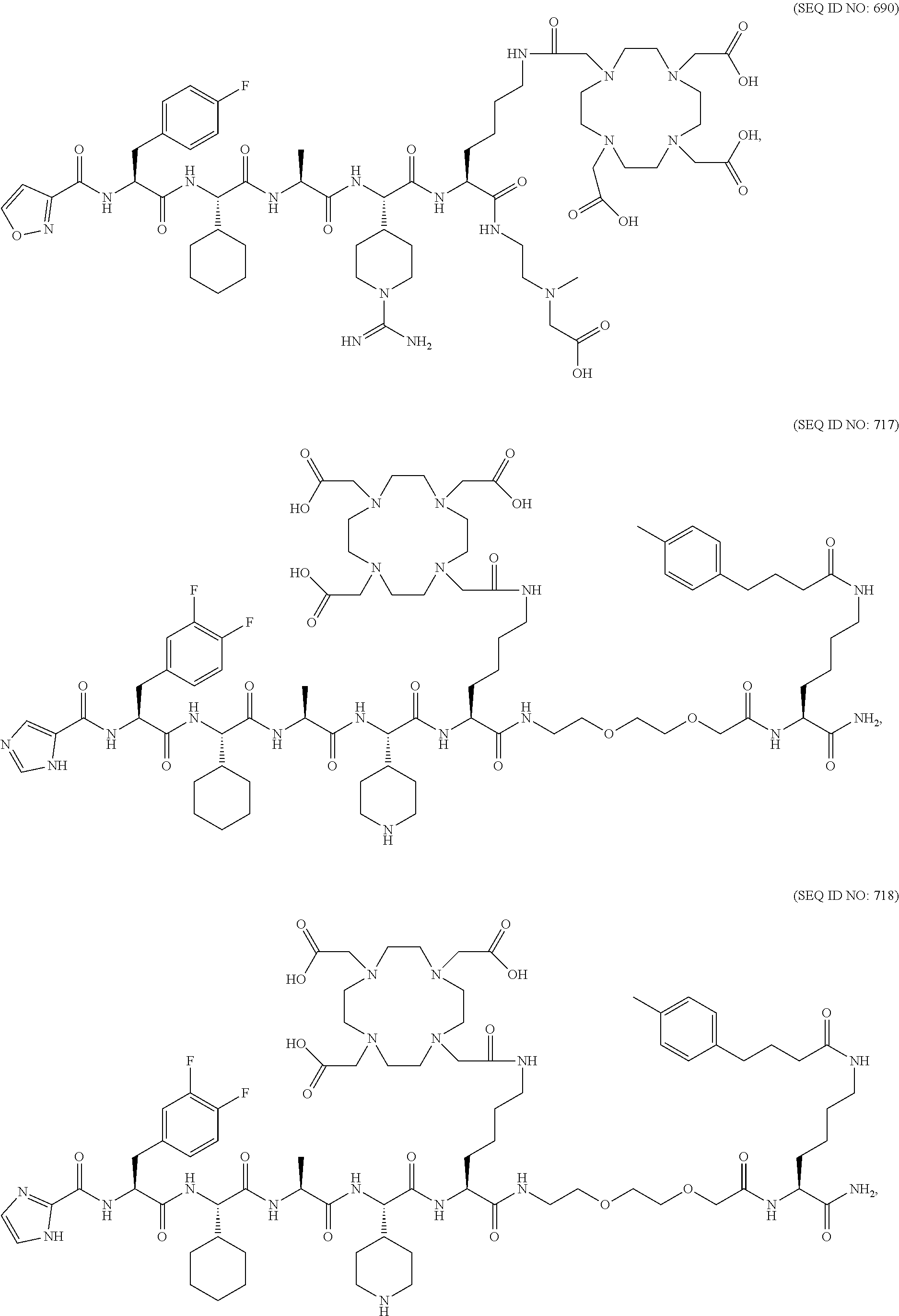 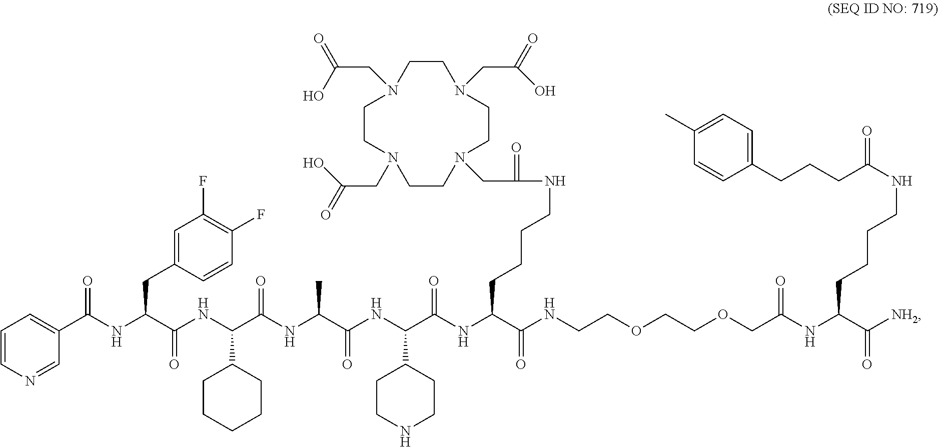 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cccnc2)C2CCCCC2)C2CCNCC2)C(N)=O)cc1] 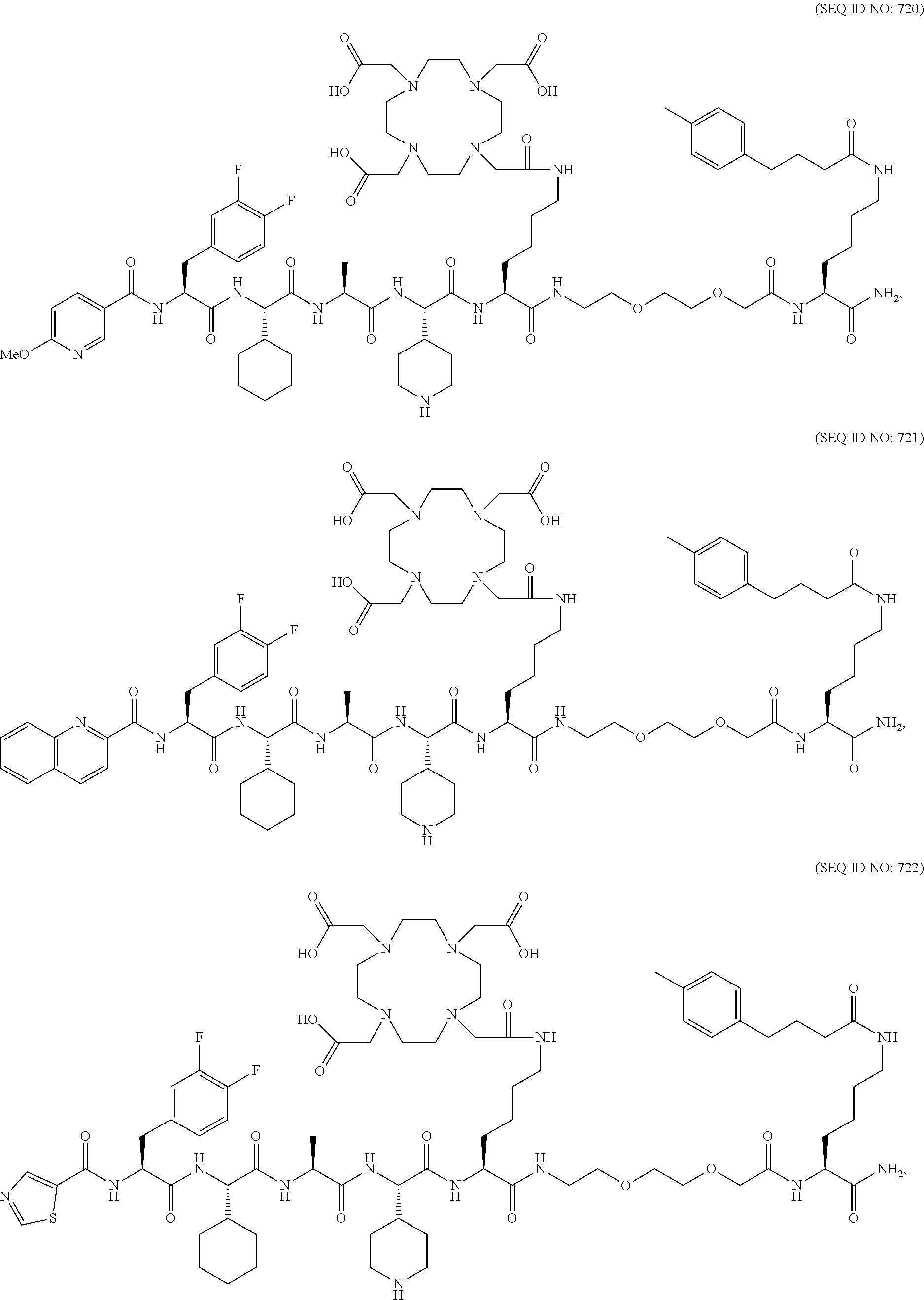 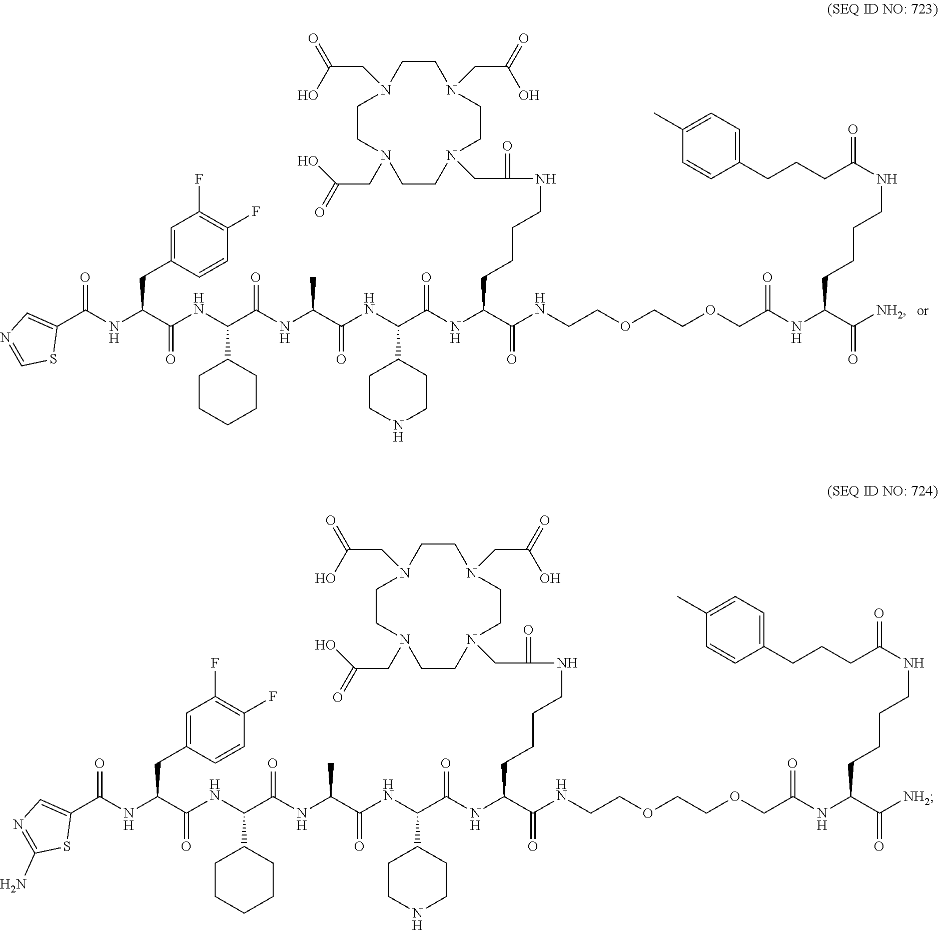 [SMILES: Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cncs2)C2CCCCC2)C2CCNCC2)C(N)=O)cc1 ; Cc1ccc(CCCC(=O)NCCCC[C@H](NC(=O)COCCOCCNC(=O)[C@H](CCCCNC(=O)CN2CCN(CC(=O)O)CCN(CC(=O)O)CCN(CC(=O)O)CC2)NC(=O)[C@@H](NC(=O)[C@H](C)NC(=O)[C@@H](NC(=O)[C@H](Cc2ccc(F)c(F)c2)NC(=O)c2cnc(N)s2)C2CCCCC2)C2CCNCC2)C(N)=O)cc1]
or a pharmaceutically acceptable salt thereof, or a radionuclide complex thereof;
wherein the radionuclide of the radionuclide complex is:
an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt); or
an α-emitting radionuclide that is 225-actinium (²²⁵Ac), 213-bismuth (²¹³Bi), 223-radium (²²³Ra), or 211-astatine (²¹¹At); or
a β-emitting radionuclide that is aluminium-[¹⁸F]fluoride ([¹⁸F]AlF), 68-gallium (⁶⁸Ga), 90-yttrium (⁹⁰Y), 177-lutetium (¹⁷⁷Lu), 161-terbium (¹⁶¹Tb), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), 52-manganese (⁵²Mn), or 212-lead (²¹²Pb); or
a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).


SEQUENCE LISTING (727 sequences)

SEQ ID NO: 1 - protein, 4 aa, synthetic construct
  SITE 1: note = (S)-cyclohexylalanine
  SITE 2: note = (S)-cyclohexylglycine
AGAR

SEQ ID NO: 2 - protein, 4 aa, synthetic construct
  SITE 1: note = (S)-2-amino-3-cyclopropylpropanoic acid
  SITE 2: note = (S)-cyclohexylglycine
AGAR

SEQ ID NO: 3 - protein, 4 aa, synthetic construct
  SITE 1: note = (S)-4-fluorophenylalanine
  SITE 2: note = (S)-cyclohexylglycine
FGAR

SEQ ID NO: 4-5 - skipped (2 entries)

SEQ ID NO: 6 - protein, 4 aa, synthetic construct
  SITE 1: note = (S)-cyclohexylalanine
  SITE 2: note = (S)-2-amino-2-cyclopentylacetic acid
AGAR

SEQ ID NO: 7 - protein, 4 aa, synthetic construct
  SITE 1: note = (S)-cyclohexylalanine
  SITE 2: note = (S)-2-amino-2-(2,3-dihydro-1H-inden-2-yl)acetic acid
AGAR

SEQ ID NO: 8 - protein, 4 aa, synthetic construct
  SITE 1: note = (S)-cyclohexylalanine
  SITE 2: note = (S)-cyclohexylalanine
AAAR

SEQ ID NO: 9-11 - skipped (3 entries)

SEQ ID NO: 12 - protein, 4 aa, synthetic construct
  SITE 1: note = (S)-cyclohexylalanine
  SITE 2: note = (S)-2-amino-2-(4,4-difluorocyclohexyl)acetic acid
AGAR

SEQ ID NO: 13 - protein, 4 aa, synthetic construct
  SITE 1: note = (S)-cyclohexylalanine
  SITE 2: note = (S)-cyclohexylglycine
  SITE 4: note = 2-amino-5-((2-aminopyridin-4-yl)amino)pentanoic acid
AGAK

SEQ ID NO: 14-16 - skipped (3 entries)

SEQ ID NO: 17 - protein, 4 aa, synthetic construct
  SITE 1: note = (S)-cyclohexylalanine
  SITE 2: note = (S)-cyclohexylglycine
  SITE 4: note = N4,N4-dimethyl-L-asparagine
AGAN

SEQ ID NO: 18-19 - skipped (2 entries)

SEQ ID NO: 20 - protein, 4 aa, synthetic construct
  SITE 1: note = (S)-cyclohexylalanine
  SITE 2: note = (S)-cyclohexylglycine
  SITE 3: note = (R)-2-amino-3,3,3-trifluoropropanoic acid
AGAR

SEQ ID NO: 21 - protein, 4 aa, synthetic construct
  SITE 1: note = (S)-cyclohexylalanine
  SITE 2: note = (S)-cyclohexylglycine
  SITE 4: note = N6-carbamimidoyl-L-lysine
AGAK

SEQ ID NO: 22 - protein, 4 aa, synthetic construct
  SITE 1: note = (S)-cyclohexylalanine
  SITE 2: note = (S)-cyclohexylglycine
  SITE 3: note = N6-diazo-L-lysine
AGKR

SEQ ID NO: 23 - protein, 4 aa, synthetic construct
  SITE 1: note = (S)-cyclohexylalanine
  SITE 2: note = (S)-cyclohexylglycine
  SITE 4: note = (S)-2-amino-3-(4-ureidophenyl)propanoic acid
AGAF

SEQ ID NO: 24 - protein, 4 aa, synthetic construct
  SITE 1: note = (S)-cyclohexylalanine
  SITE 2: note = (S)-cyclohexylglycine
  SITE 4: note = N-omega-nitro-L-arginine
AGAR

SEQ ID NO: 25 - protein, 4 aa, synthetic construct
  SITE 1: note = (S)-2-amino-3-(3-cyclopropylphenyl)propanoic acid
  SITE 2: note = (S)-cyclohexylglycine
FGAR

SEQ ID NO: 26 - protein, 4 aa, synthetic construct
  SITE 1: note = (S)-cyclohexylalanine
  SITE 2: note = (S)-cyclohexylglycine
  SITE 4: note = (S)-2-amino-3-(4-carbamoylphenyl)propanoic acid
AGAF

SEQ ID NO: 27 - protein, 4 aa, synthetic construct
  SITE 1: note = (S)-cyclohexylalanine
  SITE 2: note = (S)-cyclohexylglycine
  SITE 4: note = (S)-2-amino-3-(4-aminophenyl)propanoic acid
AGAF

SEQ ID NO: 28 - protein, 4 aa, synthetic construct
  SITE 1: note = (S)-cyclohexylalanine
  SITE 2: note = (S)-cyclohexylglycine
  SITE 4: note = (S)-2-amino-3-(4-nitrophenyl)propanoic acid
AGAF

SEQ ID NO: 29 - protein, 4 aa, synthetic construct
  SITE 1: note = (S)-cyclohexylalanine
  SITE 2: note = (S)-2-amino-3-(2-hydroxyphenyl)propanoic acid
AFAR

SEQ ID NO: 30 - skipped

SEQ ID NO: 31 - protein, 4 aa, synthetic construct
  SITE 1: note = (S)-cyclohexylalanine
  SITE 2: note = (S)-cyclohexylglycine
  SITE 4: note = (S)-2-amino-3-(4-(2-aminoethoxy)phenyl)propanoic acid
AGAF

SEQ ID NO: 32 - skipped

SEQ ID NO: 33 - protein, 4 aa, synthetic construct
  SITE 1: note = (S)-cyclohexylalanine
  SITE 2: note = (S)-cyclohexylglycine
AGAE

SEQ ID NO: 34 - skipped

SEQ ID NO: 35 - protein, 4 aa, synthetic construct
  SITE 1: note = (S)-cyclohexylalanine
  SITE 2: note = (S)-biphenylalanine
AFAR

SEQ ID NO: 36 - protein, 4 aa, synthetic construct
  SITE 1: note = (S)-cyclohexylalanine
  SITE 2: note = (S)-cyclohexylglycine
AGAF

SEQ ID NO: 37 - protein, 4 aa, synthetic construct
  SITE 1: note = (S)-cyclohexylalanine
  SITE 2: note = (S)-cyclohexylglycine
AGAN

SEQ ID NO: 38 - protein, 4 aa, synthetic construct
  SITE 1: note = (S)-cyclohexylalanine
  SITE 2: note = (S)-2-amino-2-cycloheptylacetic acid
AGAR

SEQ ID NO: 39 - protein, 4 aa, synthetic construct
  SITE 1: note = (S)-2-amino-3-cycloheptylpropanoic acid
  SITE 2: note = (S)-cyclohexylglycine
AGAR

SEQ ID NO: 40 - skipped

SEQ ID NO: 41 - protein, 4 aa, synthetic construct
  SITE 1: note = (S)-cyclohexylalanine
  SITE 2: note = (S)-2-amino-2-(2,3-dihydro-1H-inden-2-yl)acetic acid
AGSR

SEQ ID NO: 42 - protein, 4 aa, synthetic construct
  SITE 1: note = (S)-cyclohexylalanine
  SITE 2: note = (S)-2-amino-2-(2,3-dihydro-1H-inden-2-yl)acetic acid
  SITE 3: note = (S)-2-aminobutanoic acid
AGAR

SEQ ID NO: 43 - protein, 4 aa, synthetic construct
  SITE 1: note = (S)-cyclohexylalanine
  SITE 2: note = (S)-2-amino-2-(2,3-dihydro-1H-inden-2-yl)acetic acid
  SITE 3: note = (S)-2-amino-2-cyclopropylacetic acid
AGGR

SEQ ID NO: 44 - protein, 4 aa, synthetic construct
  SITE 1: note = (S)-4-fluorophenylalanine
  SITE 2: note = (S)-2-amino-2-(2,3-dihydro-1H-inden-2-yl)acetic acid
FGAR

SEQ ID NO: 45 - protein, 4 aa, synthetic construct
  SITE 1: note = 4-methoxyphenylalanine
  SITE 2: note = (S)-2-amino-2-(2,3-dihydro-1H-inden-2-yl)acetic acid
FGAR

SEQ ID NO: 46 - protein, 4 aa, synthetic construct
  SITE 2: note = (S)-2-amino-2-(2,3-dihydro-1H-inden-2-yl)acetic acid
TGAR

SEQ ID NO: 47 - protein, 4 aa, synthetic construct
  SITE 1: note = (S)-cyclohexylalanine
  SITE 2: note = (S)-cyclohexylglycine
  SITE 3: note = (S)-2-amino-6-(4-hexyl-1H-1,2,3-triazol-1-yl)hexano ic acid
AGKR

SEQ ID NO: 48 - protein, 4 aa, synthetic construct
  SITE 1: note = (S)-cyclohexylalanine
  SITE 2: note = (S)-cyclohexylglycine
  SITE 3: note = (S)-2-amino-6-(4-(cyclopentylmethyl)-1H-1,2,3-triaz ol-1-yl)hexanoic acid
AGKR

SEQ ID NO: 49 - protein, 4 aa, synthetic construct
  SITE 1: note = (S)-cyclohexylalanine
  SITE 2: note = (S)-cyclohexylglycine
  SITE 3: note = (S)-2-amino-6-(4-((2-methoxyethoxy)methyl)-1H-1,2,3 -triazol-1-yl)hexanoic acid
AGKR

SEQ ID NO: 50 - protein, 4 aa, synthetic construct
  SITE 1: note = (S)-cyclohexylalanine
  SITE 2: note = (S)-cyclohexylglycine
  SITE 3: note = (S)-2-amino-6-(4-benzyl-1H-1,2,3-triazol-1-yl)hexan oic acid
AGKR

SEQ ID NO: 51 - protein, 4 aa, synthetic construct
  SITE 1: note = 4-chlorophenylalanine
  SITE 2: note = (S)-cyclohexylglycine
FGAR

SEQ ID NO: 52 - protein, 4 aa, synthetic construct
  SITE 1: note = 3,4-difluoro-phenylalanine
  SITE 2: note = (S)-cyclohexylglycine
FGAR

SEQ ID NO: 53 - protein, 4 aa, synthetic construct
  SITE 1: note = (S)-2-amino-3,3-diphenylpropanoic acid
  SITE 2: note = (S)-cyclohexylglycine
FGAR

SEQ ID NO: 54 - protein, 4 aa, synthetic construct
  SITE 1: note = 4-bromo-L-phenylalanine
  SITE 2: note = (S)-cyclohexylglycine
FGAR

SEQ ID NO: 55 - protein, 4 aa, synthetic construct
  SITE 1: note = 4-methyl-phenylalanine
  SITE 2: note = (S)-cyclohexylglycine
FGAR

SEQ ID NO: 56 - protein, 4 aa, synthetic construct
  SITE 1: note = 4-Trifluoro-phenylalanine
  SITE 2: note = (S)-cyclohexylglycine
FGAR

SEQ ID NO: 57 - protein, 4 aa, synthetic construct
  SITE 1: note = (S)-2-amino-3-(2,4-difluorophenyl)propanoic acid
  SITE 2: note = (S)-cyclohexylglycine
FGAR

SEQ ID NO: 58 - protein, 4 aa, synthetic construct
  SITE 1: note = (S)-2-amino-3-(2,5-dichlorophenyl)propanoic acid
  SITE 2: note = (S)-cyclohexylglycine
FGAR

SEQ ID NO: 59 - protein, 4 aa, synthetic construct
  SITE 1: note = (S)-2-amino-3-(4-vinylphenyl)propanoic acid
  SITE 2: note = (S)-cyclohexylglycine
FGAR

SEQ ID NO: 60 - protein, 4 aa, synthetic construct
  SITE 1: note = (S)-2-amino-3-(3,4,5-trifluorophenyl)propanoic acid
  SITE 2: note = (S)-cyclohexylglycine
FGAR

SEQ ID NO: 61 - protein, 4 aa, synthetic construct
  SITE 1: note = (S)-2-amino-3-cyclopentylpropanoic acid
  SITE 2: note = (S)-cyclohexylglycine
AGAR

SEQ ID NO: 62 - protein, 4 aa, synthetic construct
  SITE 1: note = (S)-2-amino-3-(3,4-bis(trifluoromethyl)phenyl)propa noic acid
  SITE 2: note = (S)-cyclohexylglycine
FGAR

SEQ ID NO: 63 - protein, 4 aa, synthetic construct
  SITE 1: note = (S)-2-amino-3-(perfluorophenyl)propanoic acid
  SITE 2: note = (S)-cyclohexylglycine
FGAR

SEQ ID NO: 64 - protein, 4 aa, synthetic construct
  SITE 1: note = (S)-cyclohexylalanine
  SITE 2: note = (S)-cyclohexylglycine
  SITE 4: note = (Z)-N-omega'-methyl-L-arginine
AGAR

SEQ ID NO: 65 - protein, 4 aa, synthetic construct
  SITE 1: note = (S)-cyclohexylalanine
  SITE 2: note = (S)-cyclohexylglycine
  SITE 4: note = 2-amino-2-(piperidin-4-yl)acetic acid
AGAG

SEQ ID NO: 66-69 - skipped (4 entries)

SEQ ID NO: 70 - protein, 4 aa, synthetic construct
  SITE 1: note = (S)-cyclohexylalanine
  SITE 2: note = (S)-cyclohexylglycine
  SITE 3: note = N-methyl-Alanine
AGAR

SEQ ID NO: 71 - protein, 4 aa, synthetic construct
  SITE 1: note = (S)-cyclohexylalanine
  SITE 2: note = (S)-cyclohexylglycine
AGAR

SEQ ID NO: 72 - protein, 4 aa, synthetic construct
  SITE 1: note = (S)-cyclohexylalanine
  SITE 2: note = (S)-cyclohexylglycine
  SITE 3: note = D-N-methyl-Alanine
AGAR

SEQ ID NO: 73 - skipped

SEQ ID NO: 74 - protein, 4 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(2,4-difluorophenyl)propanoic acid
FGAF

SEQ ID NO: 75-78 - skipped (4 entries)

SEQ ID NO: 79 - protein, 4 aa, synthetic construct
  SITE 1: note = (S)-cyclohexylalanine
  SITE 2: note = (S)-cyclohexylglycine
  SITE 3: note = 2-amino-2-methylpropanoic acid
  SITE 4: note = 2-amino-2-(piperidin-4-yl)acetic acid
AGAG

SEQ ID NO: 80 - protein, 4 aa, synthetic construct
  SITE 1: note = (S)-4-fluorophenylalanine
  SITE 2: note = (S)-cyclohexylglycine
  SITE 4: note = (S)-2-amino-2-(piperidin-4-yl)acetic acid
FGAG

SEQ ID NO: 81 - protein, 4 aa, synthetic construct
  SITE 1: note = (S)-cyclohexylalanine
  SITE 2: note = (S)-cyclohexylglycine
  SITE 4: note = 2-amino-5-((2-aminopyridin-4-yl)amino)pentanoic acid
AGAK

SEQ ID NO: 82 - skipped

SEQ ID NO: 83 - protein, 4 aa, synthetic construct
  SITE 1: note = (S)-cyclohexylalanine
  SITE 2: note = (S)-cyclohexylglycine
  SITE 4: note = (S)-2-amino-2-(piperidin-4-yl)acetic acid
AGAG

SEQ ID NO: 84 - protein, 4 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(2,4-difluorophenyl)propanoic acid
AGAG

SEQ ID NO: 85 - skipped

SEQ ID NO: 86 - protein, 4 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = 2-amino-5-((2-aminopyridin-4-yl)amino)pentanoic acid
AGAK

SEQ ID NO: 87 - protein, 4 aa, synthetic construct
  SITE 1: note = (S)-cyclohexylalanine
  SITE 2: note = (S)-cyclohexylglycine
  SITE 4: note = (S)-2-amino-5-oxo-5-(piperazin-1-yl)pentanoic acid
AGAN

SEQ ID NO: 88-89 - skipped (2 entries)

SEQ ID NO: 90 - protein, 4 aa, synthetic construct
  SITE 1: note = (S)-cyclohexylalanine
  SITE 2: note = (S)-cyclohexylglycine
  SITE 4: note = N6-nicotinoyl-L-lysine
AGAK

SEQ ID NO: 91 - skipped

SEQ ID NO: 92 - protein, 4 aa, synthetic construct
  SITE 1: note = (S)-cyclohexylalanine
  SITE 2: note = (S)-cyclohexylglycine
  SITE 4: note = N6-(1-benzylpiperidine-4-carbonyl)-L-lysine
AGAK

SEQ ID NO: 93-103 - skipped (11 entries)

SEQ ID NO: 104 - protein, 4 aa, synthetic construct
  SITE 1: note = (S)-2-amino-3-cycloheptylpropanoic acid
  SITE 2: note = (S)-cyclohexylglycine
  SITE 4: note = 2-amino-5-((2-aminopyridin-4-yl)amino)pentanoic acid
AGAK

SEQ ID NO: 105 - protein, 4 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = 2-amino-5-((2-aminopyridin-4-yl)amino)pentanoic acid
AGAK

SEQ ID NO: 106 - protein, 4 aa, synthetic construct
  SITE 1: note = (S)-2-amino-3-cycloheptylpropanoic acid
  SITE 2: note = (S)-2-amino-2-cycloheptylacetic acid
  SITE 4: note = 2-amino-5-((2-aminopyridin-4-yl)amino)pentanoic acid
AGAK

SEQ ID NO: 107-108 - skipped (2 entries)

SEQ ID NO: 109 - protein, 4 aa, synthetic construct
  SITE 1: note = (S)-2-amino-3-cycloheptylpropanoic acid
  SITE 2: note = (S)-cyclohexylglycine
AGAF

SEQ ID NO: 110 - protein, 4 aa, synthetic construct
  SITE 1: note = (S)-2-amino-3-cycloheptylpropanoic acid
  SITE 2: note = (S)-cyclohexylglycine
  SITE 4: note = (S)-cyclohexylalanine
AGAA

SEQ ID NO: 111 - protein, 4 aa, synthetic construct
  SITE 1: note = (S)-2-amino-3-cycloheptylpropanoic acid
  SITE 2: note = (S)-cyclohexylglycine
  SITE 4: note = (S)-4-fluorophenylalanine
AGAF

SEQ ID NO: 112 - protein, 4 aa, synthetic construct
  SITE 1: note = (S)-2-amino-3-cycloheptylpropanoic acid
  SITE 2: note = (S)-cyclohexylglycine
  SITE 4: note = (S)-2-amino-2-phenylacetic acid
AGAG

SEQ ID NO: 113 - protein, 4 aa, synthetic construct
  SITE 1: note = (S)-2-amino-3-cycloheptylpropanoic acid
  SITE 2: note = (S)-cyclohexylglycine
  SITE 4: note = (S)-cyclohexylglycine
AGAG

SEQ ID NO: 114 - protein, 4 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-2-(piperidin-4-yl)acetic acid
AGAG

SEQ ID NO: 115 - protein, 4 aa, synthetic construct
  SITE 1: note = (S)-2-amino-3-(3-cyclopropylphenyl)propanoic acid
  SITE 2: note = (S)-cyclohexylglycine
FGAR

SEQ ID NO: 116-130 - skipped (15 entries)

SEQ ID NO: 131 - protein, 4 aa, synthetic construct
  SITE 1: note = (S)-2-amino-3-(3,4-difluorophenyl)propanoic acid
  SITE 2: note = (S)-cyclohexylglycine
FGAR

SEQ ID NO: 132 - skipped

SEQ ID NO: 133 - protein, 4 aa, synthetic construct
  SITE 1: note = (S)-2-amino-3-(2,5-difluorophenyl)propanoic acid
  SITE 2: note = (S)-cyclohexylglycine
FGAR

SEQ ID NO: 134-135 - skipped (2 entries)

SEQ ID NO: 136 - protein, 4 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
AGAF

SEQ ID NO: 137 - protein, 4 aa, synthetic construct
  SITE 1: note = (S)-2-amino-3-(3,4-difluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
FGAF

SEQ ID NO: 138 - skipped

SEQ ID NO: 139 - protein, 4 aa, synthetic construct
  SITE 2: note = (S)-2-amino-2-(2,3-dihydro-1H-inden-2-yl)acetic acid
FGAR

SEQ ID NO: 140 - protein, 4 aa, synthetic construct
  SITE 1: note = (S)-2-amino-3-(2,5-dichlorophenyl)propanoic acid
  SITE 2: note = (S)-cyclohexylglycine
FGAR

SEQ ID NO: 141-146 - skipped (6 entries)

SEQ ID NO: 147 - protein, 4 aa, synthetic construct
  SITE 1: note = (S)-2-amino-3-(3,4,5-trifluorophenyl)propanoic acid
  SITE 2: note = (S)-cyclohexylglycine
  SITE 4: note = (S)-2-amino-2-(piperidin-4-yl)acetic acid
FGAG

SEQ ID NO: 148 - protein, 4 aa, synthetic construct
  SITE 1: note = (S)-2-amino-3-cyclopentylpropanoic acid
  SITE 2: note = (S)-cyclohexylglycine
  SITE 4: note = (S)-2-amino-2-(piperidin-4-yl)acetic acid
AGAG

SEQ ID NO: 149 - protein, 4 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
FGAG

SEQ ID NO: 150 - skipped

SEQ ID NO: 151 - protein, 4 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(piperidin-4-yl)acetic acid
FFAG

SEQ ID NO: 152 - protein, 4 aa, synthetic construct
  SITE 1: note = (S)-2-amino-3-cycloheptylpropanoic acid
  SITE 2: note = (S)-biphenylalanine
  SITE 4: note = (S)-2-amino-2-(piperidin-4-yl)acetic acid
AFAG

SEQ ID NO: 153 - protein, 5 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = Amidated Lysine
AGAXK

SEQ ID NO: 154 - protein, 5 aa, synthetic construct
  SITE 1: note = (S)-2-amino-3-cycloheptylpropanoic acid
  SITE 2: note = (S)-2-amino-2-cycloheptylacetic acid
  SITE 4: note = (S)-2-amino-2-(1-carbamimidoylpiperidin-4-yl)acetic acid
  SITE 5: note = Amidated Lysine
AGAXK

SEQ ID NO: 155-156 - skipped (2 entries)

SEQ ID NO: 157 - protein, 5 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(piperidin-4-yl)acetic acid
  SITE 5: note = Amidated Lysine
FGAGK

SEQ ID NO: 158 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 6: note = Amidated 6-aminohexanoic acid
AGAXKX

SEQ ID NO: 159 - protein, 6 aa, synthetic construct
  SITE 1: note = (S)-cyclohexylalanine
  SITE 2: note = (S)-cyclohexylglycine
  SITE 5: note = (S)-2-amino-3-(3-cyclopropylphenyl)propanoic acid
  SITE 6: note = Amidated Lysine
AGARFK

SEQ ID NO: 160 - protein, 6 aa, synthetic construct
  SITE 1: note = (S)-cyclohexylalanine
  SITE 2: note = (S)-cyclohexylglycine
  SITE 4: note = (S)-2-amino-2-(piperidin-4-yl)acetic acid
  SITE 5: note = 3-(2-(2-aminoethoxy)ethoxy)propanoic acid
  SITE 6: note = Amidated Lysine
AGAGXK

SEQ ID NO: 161 - protein, 5 aa, synthetic construct
  SITE 1: note = (S)-cyclohexylalanine
  SITE 2: note = (S)-cyclohexylglycine
  SITE 5: note = Amidated Lysine
AGARK

SEQ ID NO: 162 - protein, 6 aa, synthetic construct
  SITE 6: note = Amidated Ornithine
LIGRIX

SEQ ID NO: 163 - protein, 5 aa, synthetic construct
  SITE 1: note = (S)-cyclohexylalanine
  SITE 2: note = (S)-cyclohexylglycine
  SITE 5: note = Amidated Ornithine
AGARX

SEQ ID NO: 164 - protein, 5 aa, synthetic construct
  SITE 1: note = (S)-cyclohexylalanine
  SITE 2: note = (S)-cyclohexylglycine
  SITE 5: note = Amidated Ornithine
AGARX

SEQ ID NO: 165 - protein, 5 aa, synthetic construct
  SITE 1: note = (S)-cyclohexylalanine
  SITE 2: note = (S)-cyclohexylglycine
  SITE 5: note = Amidated Ornithine
AGARX

SEQ ID NO: 166 - protein, 5 aa, synthetic construct
LIGRL

SEQ ID NO: 167 - protein, 5 aa, synthetic construct
  SITE 1: note = (S)-2-amino-4-methylpent-4-enoic acid
  SITE 1: note = Amidated Leucine
XIGRL

SEQ ID NO: 168 - protein, 5 aa, synthetic construct
  SITE 1: note = (S)-cyclohexylalanine
  SITE 2: note = (S)-cyclohexylglycine
  SITE 5: note = Amidated N6-acetyl-L-lysine
AGARK

SEQ ID NO: 169 - protein, 5 aa, synthetic construct
  SITE 1: note = (2S)-2-((1r,3R,5S)-adamantan-1-yl)-2-aminoacetic acid
  SITE 2: note = (S)-cyclohexylglycine
  SITE 5: note = Amidated lysine
GGARK

SEQ ID NO: 170 - protein, 5 aa, synthetic construct
  SITE 1: note = (S)-2-amino-3-cyclopropylpropanoic acid
  SITE 2: note = (S)-cyclohexylglycine
  SITE 5: note = Amidated lysine
AGARK

SEQ ID NO: 171 - protein, 5 aa, synthetic construct
  SITE 1: note = (S)-2-amino-3-(5,6,7,8-tetrahydronaphthalen-2-yl)pr opanoic acid
  SITE 2: note = (S)-cyclohexylglycine
  SITE 5: note = Amidated lysine
FGARK

SEQ ID NO: 172 - protein, 5 aa, synthetic construct
  SITE 1: note = (S)-2-amino-3-(4-fluorophenyl)propanoic acid
  SITE 2: note = (S)-cyclohexylglycine
  SITE 5: note = Amidated lysine
FGARK

SEQ ID NO: 173 - protein, 5 aa, synthetic construct
  SITE 1: note = (S)-2-amino-2-((R)-2,3-dihydro-1H-inden-1-yl)acetic acid
  SITE 2: note = (S)-cyclohexylglycine
  SITE 5: note = Amidated lysine
GGARK

SEQ ID NO: 174 - protein, 5 aa, synthetic construct
  SITE 1: note = (S)-cyclohexylalanine
  SITE 2: note = (S)-cyclohexylglycine
  SITE 3: note = (S)-2,3-diaminopropanoic acid
  SITE 5: note = Amidated lysine
AGXRK

SEQ ID NO: 175 - protein, 5 aa, synthetic construct
  SITE 1: note = (S)-cyclohexylglycine
  SITE 2: note = (S)-cyclohexylglycine
  SITE 5: note = Amidated lysine
GAARK

SEQ ID NO: 176 - protein, 5 aa, synthetic construct
  SITE 1: note = (S)-cyclohexylalanine
  SITE 2: note = (S)-cyclohexylglycine
  SITE 3: note = Ornithine
  SITE 5: note = Amidated lysine
AGXRK

SEQ ID NO: 177 - protein, 5 aa, synthetic construct
  SITE 1: note = (S)-cyclohexylalanine
  SITE 2: note = (S)-cyclohexylglycine
  SITE 5: note = Amidated lysine
AGERK

SEQ ID NO: 178 - protein, 5 aa, synthetic construct
  SITE 1: note = (S)-cyclohexylalanine
  SITE 2: note = (S)-2-amino-3-ethylpentanoic acid
  SITE 5: note = Amidated lysine
AXARK

SEQ ID NO: 179 - protein, 5 aa, synthetic construct
  SITE 1: note = (S)-cyclohexylalanine
  SITE 2: note = (S)-2-amino-4,4,4-trifluoro-3-(trifluoro-l5-methyl) -4l5-butanoic acid
  SITE 5: note = Amidated lysine
AAARK

SEQ ID NO: 180 - protein, 5 aa, synthetic construct
  SITE 1: note = (S)-cyclohexylalanine
  SITE 2: note = (S)-2-amino-4,4-dimethylpentanoic acid
  SITE 5: note = Amidated lysine
AAARK

SEQ ID NO: 181 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 182 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 5: note = Amidated lysine
AGARK

SEQ ID NO: 183 - protein, 5 aa, synthetic construct
  SITE 1: note = (S)-cyclohexylglycine
  SITE 2: note = (S)-cyclohexylglycine
  SITE 5: note = Amidated lysine
GGARK

SEQ ID NO: 184 - protein, 5 aa, synthetic construct
  SITE 1: note = (S)-cyclohexylalanine
  SITE 2: note = 2-amino-2,3-dihydro-1H-indene-2-carboxylic acid
  SITE 5: note = Amidated lysine
AXARK

SEQ ID NO: 185 - protein, 5 aa, synthetic construct
  SITE 1: note = (S)-cyclohexylalanine
  SITE 2: note = 1-aminocyclopentane-1-carboxylic acid
  SITE 5: note = Amidated lysine
AXARK

SEQ ID NO: 186 - protein, 5 aa, synthetic construct
  SITE 1: note = (S)-cyclohexylalanine
  SITE 2: note = 1-aminocyclohexane-1-carboxylic acid
  SITE 5: note = Amidated lysine
AXARK

SEQ ID NO: 187 - protein, 5 aa, synthetic construct
  SITE 1: note = (S)-cyclohexylalanine
  SITE 2: note = 4-aminopiperidine-4-carboxylic acid
  SITE 5: note = Amidated lysine
AXARK

SEQ ID NO: 188 - protein, 5 aa, synthetic construct
  SITE 1: note = (S)-cyclohexylalanine
  SITE 2: note = (S)-cyclohexylglycine
  SITE 5: note = Amidated lysine
AGQRK

SEQ ID NO: 189 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 190 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 191 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 192 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 193 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 194 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 195 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 196 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 197 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 198 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 199 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 200 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 201 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 202 - protein, 5 aa, synthetic construct
  SITE 1: note = (S)-cyclohexylalanine
  SITE 2: note = (S)-2-amino-3-ethylpentanoic acid
  SITE 5: note = Amidated lysine
AXARK

SEQ ID NO: 203 - protein, 5 aa, synthetic construct
  SITE 1: note = (S)-cyclohexylalanine
  SITE 2: note = (S)-2-amino-2-(4-fluorophenyl)acetic acid
  SITE 5: note = Amidated lysine
AXARK

SEQ ID NO: 204 - protein, 5 aa, synthetic construct
  SITE 1: note = (2S)-2-((1r,3R,5S)-adamantan-1-yl)-2-aminoacetic acid
  SITE 2: note = (S)-cyclohexylglycine
  SITE 5: note = Amidated lysine
GGARK

SEQ ID NO: 205 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 206 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 207 - protein, 5 aa, synthetic construct
  SITE 1: note = (S)-cyclohexylalanine
  SITE 2: note = (S)-2-amino-2-(4,4-difluorocyclohexyl)acetic acid
  SITE 5: note = Amidated lysine
AGARK

SEQ ID NO: 208-212 - skipped (5 entries)

SEQ ID NO: 213 - protein, 5 aa, synthetic construct
  SITE 1: note = (S)-3-([1,1'-biphenyl]-4-yl)-2-aminopropanoic acid
  SITE 2: note = (S)-cyclohexylglycine
  SITE 5: note = Amidated lysine
FGARK

SEQ ID NO: 214 - protein, 5 aa, synthetic construct
  SITE 1: note = (S)-cyclohexylalanine
  SITE 2: note = 2-amino-2-methylpropanoic acid
  SITE 5: note = Amidated lysine
AAARK

SEQ ID NO: 215 - protein, 5 aa, synthetic construct
  SITE 1: note = (S)-cyclohexylalanine
  SITE 2: note = (S)-cyclohexylglycine
  SITE 4: note = N4,N4-dimethyl-L-asparagine
  SITE 5: note = Amidated lysine
AGANK

SEQ ID NO: 216 - protein, 5 aa, synthetic construct
  SITE 1: note = (S)-cyclohexylalanine
  SITE 2: note = (S)-cyclohexylglycine
  SITE 4: note = (S)-2-amino-3-(6-aminopyridin-3-yl)propanoic acid
  SITE 5: note = Amidated lysine
AGAXK

SEQ ID NO: 217 - protein, 5 aa, synthetic construct
  SITE 1: note = (S)-cyclohexylalanine
  SITE 2: note = (S)-cyclohexylglycine
  SITE 4: note = (S)-2-amino-5-ureidopentanoic acid
  SITE 5: note = Amidated lysine
AGAXK

SEQ ID NO: 218 - protein, 5 aa, synthetic construct
  SITE 1: note = (S)-cyclohexylalanine
  SITE 2: note = (S)-cyclohexylglycine
  SITE 4: note = N6-octanoyl-L-lysine
  SITE 5: note = Amidated lysine
AGARK

SEQ ID NO: 219 - protein, 6 aa, synthetic construct
  SITE 1: note = (S)-cyclohexylalanine
  SITE 2: note = (S)-cyclohexylglycine
  SITE 5: note = (S)-2-aminoheptanoic acid
  SITE 5: note = Amidated lysine
AGARXK

SEQ ID NO: 220 - protein, 5 aa, synthetic construct
  SITE 1: note = (S)-cyclohexylalanine
  SITE 2: note = (S)-cyclohexylglycine
  SITE 3: note = (R)-2-amino-3,3,3-trifluoropropanoic acid
  SITE 5: note = Amidated lysine
AGARK

SEQ ID NO: 221 - protein, 5 aa, synthetic construct
  SITE 1: note = (S)-cyclohexylalanine
  SITE 2: note = (S)-cyclohexylglycine
  SITE 5: note = Amidated N6-(pent-4-ynoyl)-L-lysine
AGARK

SEQ ID NO: 222 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 223 - protein, 5 aa, synthetic construct
  SITE 1: note = (S)-cyclohexylalanine
  SITE 2: note = (S)-cyclohexylglycine
  SITE 5: note = Amidated N6-carbamimidoyl-L-lysine
AGAKK

SEQ ID NO: 224 - protein, 5 aa, synthetic construct
  SITE 1: note = (S)-cyclohexylalanine
  SITE 2: note = (S)-cyclohexylglycine
  SITE 5: note = Amidated (S)-2-amino-3-(4-azidophenyl)propanoic acid
AGARF

SEQ ID NO: 225 - protein, 5 aa, synthetic construct
  SITE 1: note = (S)-cyclohexylalanine
  SITE 2: note = (S)-cyclohexylglycine
  SITE 5: note = Amidated (S)-2-amino-3-(4-(azidomethyl)phenyl)propanoic acid
AGARF

SEQ ID NO: 226 - protein, 5 aa, synthetic construct
  SITE 1: note = (S)-cyclohexylalanine
  SITE 2: note = (S)-cyclohexylglycine
  SITE 3: note = Amidated N6-diazo-L-lysine
AGKRK

SEQ ID NO: 227 - protein, 5 aa, synthetic construct
  SITE 1: note = (S)-cyclohexylalanine
  SITE 2: note = (S)-cyclohexylglycine
  SITE 4: note = (S)-2-amino-3-(4-guanidinophenyl)propanoic acid
  SITE 5: note = Amidated lysine
AGAFK

SEQ ID NO: 228 - protein, 5 aa, synthetic construct
  SITE 1: note = (S)-cyclohexylalanine
  SITE 2: note = (S)-cyclohexylglycine
  SITE 4: note = Amidated N-omega-nitro-L-arginine
AGARK

SEQ ID NO: 229 - protein, 5 aa, synthetic construct
  SITE 1: note = (S)-cyclohexylalanine
  SITE 2: note = (S)-cyclohexylglycine
  SITE 4: note = (S)-2-amino-3-(4-((R)-2,6-dioxohexahydropyrimidine- 4-carboxamido)phenyl)propanoic acid
  SITE 5: note = Amidated lysine
AGAXK

SEQ ID NO: 230 - protein, 5 aa, synthetic construct
  SITE 1: note = (S)-cyclohexylalanine
  SITE 2: note = (S)-cyclohexylglycine
  SITE 4: note = (E)-N-omega,N-omega'-diethyl-L-arginine
  SITE 5: note = Amidated lysine
AGARK

SEQ ID NO: 231 - protein, 5 aa, synthetic construct
  SITE 1: note = (S)-cyclohexylalanine
  SITE 2: note = (S)-cyclohexylglycine
  SITE 4: note = (E)-N-omega,N-omega'-dimethyl-L-arginine
  SITE 5: note = Amidated lysine
AGARK

SEQ ID NO: 232 - protein, 5 aa, synthetic construct
  SITE 1: note = (S)-2-amino-3-(3-cyclopropylphenyl)propanoic acid
  SITE 2: note = (S)-cyclohexylglycine
  SITE 5: note = Amidated lysine
FGARK

SEQ ID NO: 233 - protein, 5 aa, synthetic construct
  SITE 1: note = (S)-cyclohexylalanine
  SITE 2: note = (S)-cyclohexylglycine
  SITE 4: note = (S)-2-amino-3-(4-guanidinophenyl)propanoic acid
  SITE 5: note = Amidated lysine
AGAFK

SEQ ID NO: 234 - protein, 5 aa, synthetic construct
  SITE 1: note = (S)-cyclohexylalanine
  SITE 2: note = (S)-cyclohexylglycine
  SITE 4: note = (S)-2-amino-3-(4-carbamoylphenyl)propanoic acid
  SITE 5: note = Amidated lysine
AGAFK

SEQ ID NO: 235 - protein, 5 aa, synthetic construct
  SITE 1: note = (S)-cyclohexylalanine
  SITE 2: note = (S)-cyclohexylglycine
  SITE 4: note = (S)-2-amino-3-(4-aminophenyl)propanoic acid
  SITE 5: note = Amidated lysine
AGAFK

SEQ ID NO: 236 - protein, 5 aa, synthetic construct
  SITE 1: note = (S)-cyclohexylalanine
  SITE 2: note = (S)-cyclohexylglycine
  SITE 4: note = (S)-2-amino-3-(4-nitrophenyl)propanoic acid
  SITE 5: note = Amidated lysine
AGAFK

SEQ ID NO: 237 - protein, 5 aa, synthetic construct
  SITE 1: note = (S)-cyclohexylalanine
  SITE 2: note = (S)-2-amino-3-(2-hydroxyphenyl)propanoic acid
  SITE 5: note = Amidated lysine
AFARK

SEQ ID NO: 238 - protein, 5 aa, synthetic construct
  SITE 1: note = (S)-2-amino-3-methyl-3-phenylbutanoic acid
  SITE 2: note = (S)-cyclohexylglycine
  SITE 5: note = Amidated lysine
XGARK

SEQ ID NO: 239 - protein, 5 aa, synthetic construct
  SITE 1: note = (R)-2-amino-3-methyl-3-phenylbutanoic acid
  SITE 2: note = (S)-cyclohexylglycine
  SITE 5: note = Amidated lysine
XGARK

SEQ ID NO: 240 - protein, 5 aa, synthetic construct
  SITE 1: note = (S)-2-amino-3-(4-(tert-butyl)phenyl)propanoic acid
  SITE 2: note = (S)-cyclohexylglycine
  SITE 5: note = Amidated lysine
FGARK

SEQ ID NO: 241 - protein, 5 aa, synthetic construct
  SITE 1: note = (S)-cyclohexylalanine
  SITE 2: note = (S)-3-(4-acetylphenyl)-2-aminopropanoic acid
  SITE 5: note = Amidated lysine
AFARK

SEQ ID NO: 242 - protein, 5 aa, synthetic construct
  SITE 2: note = (S)-cyclohexylglycine
  SITE 5: note = Amidated lysine
WGARK

SEQ ID NO: 243 - protein, 5 aa, synthetic construct
  SITE 1: note = (S)-cyclohexylalanine
  SITE 2: note = (S)-cyclohexylglycine
  SITE 4: note = (S)-3-amino-6-guanidinohexanoic acid
  SITE 5: note = Amidated lysine
AGAXK

SEQ ID NO: 244 - protein, 5 aa, synthetic construct
  SITE 1: note = (S)-3-amino-4-cyclohexylbutanoic acid
  SITE 2: note = (S)-cyclohexylglycine
  SITE 5: note = Amidated lysine
XGARK

SEQ ID NO: 245 - protein, 5 aa, synthetic construct
  SITE 1: note = (R)-3-amino-3-(naphthalen-1-yl)propanoic acid
  SITE 2: note = (S)-cyclohexylglycine
  SITE 5: note = Amidated lysine
XGARK

SEQ ID NO: 246 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 247 - protein, 5 aa, synthetic construct
  SITE 1: note = (S)-cyclohexylalanine
  SITE 2: note = (S)-cyclohexylglycine
  SITE 4: note = 3-amino-3-(piperidin-3-yl)propanoic acid
  SITE 5: note = Amidated lysine
AGAXK

SEQ ID NO: 248 - protein, 5 aa, synthetic construct
  SITE 1: note = (S)-cyclohexylalanine
  SITE 2: note = (S)-cyclohexylglycine
  SITE 4: note = 3-amino-3-(1-carbamimidoylpiperidin-3-yl)propanoic acid
  SITE 5: note = Amidated lysine
AGAXK

SEQ ID NO: 249 - protein, 5 aa, synthetic construct
  SITE 1: note = (S)-cyclohexylalanine
  SITE 2: note = (S)-cyclohexylglycine
  SITE 3: note = 1-amino-4-oxocyclohexane-1-carboxylic acid
  SITE 5: note = Amidated lysine
AGXRK

SEQ ID NO: 250 - protein, 5 aa, synthetic construct
  SITE 1: note = (S)-cyclohexylalanine
  SITE 2: note = (S)-cyclohexylglycine
  SITE 5: note = Amidated lysine
AGPRK

SEQ ID NO: 251 - protein, 5 aa, synthetic construct
  SITE 1: note = (S)-cyclohexylalanine
  SITE 2: note = (S)-cyclohexylglycine
  SITE 4: note = (S)-2-amino-3-(4-(2-aminoethoxy)phenyl)propanoic acid
  SITE 5: note = Amidated lysine
AGAFK

SEQ ID NO: 252 - protein, 5 aa, synthetic construct
  SITE 1: note = (S)-cyclohexylalanine
  SITE 2: note = (S)-cyclohexylglycine
  SITE 4: note = (S)-2-aminohexanoic acid
  SITE 5: note = Amidated lysine
AGAXK

SEQ ID NO: 253 - protein, 5 aa, synthetic construct
  SITE 1: note = (S)-cyclohexylalanine
  SITE 2: note = (S)-cyclohexylglycine
  SITE 5: note = Amidated lysine
AGAEK

SEQ ID NO: 254 - protein, 5 aa, synthetic construct
  SITE 1: note = (S)-cyclohexylalanine
  SITE 2: note = (S)-cyclohexylglycine
  SITE 5: note = Amidated lysine
AGAPK

SEQ ID NO: 255 - protein, 6 aa, synthetic construct
  SITE 1: note = (S)-cyclohexylalanine
  SITE 2: note = (S)-cyclohexylglycine
  SITE 5: note = Amidated lysine
AGARYK

SEQ ID NO: 256 - protein, 5 aa, synthetic construct
  SITE 1: note = 1-aminocyclohexane-1-carboxylic acid
  SITE 2: note = (S)-cyclohexylglycine
  SITE 5: note = Amidated lysine
XGARK

SEQ ID NO: 257 - protein, 5 aa, synthetic construct
  SITE 1: note = 2-amino-2,3-dihydro-1H-indene-2-carboxylic acid
  SITE 2: note = (S)-cyclohexylglycine
  SITE 5: note = Amidated lysine
XGARK

SEQ ID NO: 258 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 259 - protein, 5 aa, synthetic construct
  SITE 1: note = (S)-cyclohexylalanine
  SITE 2: note = (S)-2-([1,1'-biphenyl]-4-yl)-2-aminoacetic acid
  SITE 5: note = Amidated lysine
AFARK

SEQ ID NO: 260 - protein, 5 aa, synthetic construct
  SITE 1: note = (S)-cyclohexylalanine
  SITE 2: note = (R)-2-([1,1'-biphenyl]-4-yl)-2-aminoacetic acid
  SITE 5: note = Amidated lysine
AGARK

SEQ ID NO: 261 - protein, 5 aa, synthetic construct
  SITE 1: note = (S)-cyclohexylalanine
  SITE 2: note = (S)-cyclohexylglycine
  SITE 5: note = Amidated lysine
AGAFK

SEQ ID NO: 262 - protein, 5 aa, synthetic construct
  SITE 1: note = (S)-cyclohexylalanine
  SITE 2: note = (S)-cyclohexylglycine
  SITE 5: note = Amidated lysine
AGANK

SEQ ID NO: 263 - protein, 5 aa, synthetic construct
  SITE 1: note = (S)-cyclohexylalanine
  SITE 2: note = (S)-2-amino-2-cycloheptylacetic acid
  SITE 5: note = Amidated lysine
AGARK

SEQ ID NO: 264 - protein, 5 aa, synthetic construct
  SITE 1: note = (S)-2-amino-3-cycloheptylpropanoic acid
  SITE 2: note = (S)-cyclohexylglycine
  SITE 5: note = Amidated lysine
AGARK

SEQ ID NO: 265 - skipped

SEQ ID NO: 266 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 5: note = Amidated Ornithine
AGARX

SEQ ID NO: 267 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 5: note = Amidated Ornithine
AGARX

SEQ ID NO: 268 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 3: note = (S)-2-aminobutanoic acid
  SITE 5: note = Amidated Ornithine
AGARX

SEQ ID NO: 269 - skipped

SEQ ID NO: 270 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 3: note = (S)-2-amino-2-cyclopropylacetic acid
  SITE 5: note = Amidated Ornithine
AGGRX

SEQ ID NO: 271 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 272 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 273 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 274 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 275 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 276 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 277 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 278 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 279 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 280 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 281 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 282 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 283 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 284 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 285 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 286 - protein, 5 aa, synthetic construct
  SITE 1: note = (S)-2-amino-3-(4-methoxyphenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 287 - protein, 5 aa, synthetic construct
  SITE 1: note = (S)-2-amino-3-(4-aminophenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 288 - protein, 5 aa, synthetic construct
  SITE 1: note = (S)-2-amino-3-(4-carbamoylphenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 289 - protein, 5 aa, synthetic construct
  SITE 1: note = (S)-2-amino-3-(4-guanidinophenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 290 - protein, 5 aa, synthetic construct
  SITE 1: note = (S)-3-(4-acetylphenyl)-2-aminopropanoic acid
  SITE 2: note = (S)-2-amino-2-(2,3-dihydro-1H-inden-2-yl)acetic acid
  SITE 5: note = Amidated lysine
FGARK

SEQ ID NO: 291 - protein, 5 aa, synthetic construct
  SITE 1: note = (S)-2-amino-3-(4-azidophenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 292 - protein, 5 aa, synthetic construct
  SITE 1: note = (S)-2-amino-3-(4-(azidomethyl)phenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 293 - protein, 5 aa, synthetic construct
  SITE 2: note = (S)-2-amino-2-(2,3-dihydro-1H-inden-2-yl)acetic acid
  SITE 5: note = Amidated lysine
FGARK

SEQ ID NO: 294 - protein, 5 aa, synthetic construct
  SITE 2: note = (S)-2-amino-2-(2,3-dihydro-1H-inden-2-yl)acetic acid
  SITE 5: note = Amidated lysine
YGARK

SEQ ID NO: 295 - protein, 5 aa, synthetic construct
  SITE 2: note = (S)-2-amino-2-(2,3-dihydro-1H-inden-2-yl)acetic acid
  SITE 5: note = Amidated lysine
YGARK

SEQ ID NO: 296 - protein, 5 aa, synthetic construct
  SITE 2: note = (S)-2-amino-2-(2,3-dihydro-1H-inden-2-yl)acetic acid
  SITE 5: note = Amidated lysine
YGARK

SEQ ID NO: 297 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 298 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 299 - protein, 5 aa, synthetic construct
  SITE 1: note = (S)-2-amino-3-(4-azidophenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 300 - protein, 5 aa, synthetic construct
  SITE 1: note = (S)-2-amino-3-(4-azidophenyl)propanoic acid
  SITE 2: note = (S)-2-amino-2-(2,3-dihydro-1H-inden-2-yl)acetic acid
  SITE 5: note = Amidated lysine
FGARK

SEQ ID NO: 301 - protein, 5 aa, synthetic construct
  SITE 1: note = (S)-3-(4-acetylphenyl)-2-aminopropanoic acid
  SITE 2: note = (S)-2-amino-2-(2,3-dihydro-1H-inden-2-yl)acetic acid
  SITE 5: note = Amidated lysine
FGARK

SEQ ID NO: 302 - protein, 5 aa, synthetic construct
  SITE 1: note = (S)-3-(4-acetylphenyl)-2-aminopropanoic acid
  SITE 2: note = (S)-2-amino-2-(2,3-dihydro-1H-inden-2-yl)acetic acid
  SITE 5: note = Amidated lysine
FGARK

SEQ ID NO: 303 - protein, 5 aa, synthetic construct
  SITE 1: note = (S)-cyclohexylalanine
  SITE 2: note = (S)-cyclohexylglycine
  SITE 3: note = (S)-2-amino-6-(4-hexyl-1H-1,2,3-triazol-1-yl)hexano ic acid
  SITE 5: note = Amidated lysine
AGKRK

SEQ ID NO: 304 - protein, 5 aa, synthetic construct
  SITE 1: note = (S)-cyclohexylalanine
  SITE 2: note = (S)-cyclohexylglycine
  SITE 3: note = (S)-2-amino-6-(4-(cyclopentylmethyl)-1H-1,2,3-triaz ol-1-yl)hexanoic acid
  SITE 5: note = Amidated lysine
AGKRK

SEQ ID NO: 305 - protein, 5 aa, synthetic construct
  SITE 1: note = (S)-cyclohexylalanine
  SITE 2: note = (S)-cyclohexylglycine
  SITE 3: note = (S)-2-amino-6-(4-((2-methoxyethoxy)methyl)-1H-1,2,3 -triazol-1-yl)hexanoic acid
  SITE 5: note = Amidated lysine
AGKRK

SEQ ID NO: 306 - protein, 5 aa, synthetic construct
  SITE 1: note = (S)-cyclohexylalanine
  SITE 2: note = (S)-cyclohexylglycine
  SITE 3: note = (S)-2-amino-6-(4-benzyl-1H-1,2,3-triazol-1-yl)hexan oic acid
  SITE 5: note = Amidated lysine
AGKRK

SEQ ID NO: 307 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 308 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 309 - protein, 5 aa, synthetic construct
  SITE 1: note = (S)-cyclohexylalanine
  SITE 2: note = (S)-cyclohexylglycine
  SITE 5: note = Amidated Ornithine
AGARX

SEQ ID NO: 310 - protein, 5 aa, synthetic construct
  SITE 1: note = (S)-cyclohexylalanine
  SITE 2: note = (S)-cyclohexylglycine
  SITE 5: note = Amidated Ornithine
AGARX

SEQ ID NO: 311 - protein, 5 aa, synthetic construct
  SITE 1: note = (S)-cyclohexylalanine
  SITE 2: note = (S)-cyclohexylglycine
  SITE 5: note = Amidated Ornithine
AGARX

SEQ ID NO: 312 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 313 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 314 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 315 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 316 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 317 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 318 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 319 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 320 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 321 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 322 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 323 - protein, 5 aa, synthetic construct
  SITE 1: note = (S)-cyclohexylalanine
  SITE 2: note = (S)-cyclohexylglycine
  SITE 4: note = (S)-2-amino-3-(piperidin-1-yl)propanoic acid
  SITE 5: note = Amidated lysine
AGAXK

SEQ ID NO: 324 - protein, 5 aa, synthetic construct
  SITE 1: note = (S)-2-amino-3-(piperidin-1-yl)propanoic acid
  SITE 2: note = (S)-cyclohexylglycine
  SITE 5: note = Amidated lysine
XGARK

SEQ ID NO: 325 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 326 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 327 - protein, 5 aa, synthetic construct
  SITE 1: note = (S)-2-amino-3-(4-chlorophenyl)propanoic acid
  SITE 2: note = (S)-cyclohexylglycine
  SITE 5: note = Amidated lysine
FGARK

SEQ ID NO: 328 - protein, 5 aa, synthetic construct
  SITE 1: note = (S)-2-amino-3-(3,4-difluorophenyl)propanoic acid
  SITE 2: note = (S)-cyclohexylglycine
  SITE 5: note = Amidated lysine
FGARK

SEQ ID NO: 329 - protein, 5 aa, synthetic construct
  SITE 1: note = (S)-2-amino-3,3-diphenylpropanoic acid
  SITE 2: note = (S)-cyclohexylglycine
  SITE 5: note = Amidated lysine
FGARK

SEQ ID NO: 330 - protein, 5 aa, synthetic construct
  SITE 1: note = (S)-2-amino-3-(4-bromophenyl)propanoic acid
  SITE 2: note = (S)-cyclohexylglycine
  SITE 5: note = Amidated lysine
FGARK

SEQ ID NO: 331 - protein, 5 aa, synthetic construct
  SITE 1: note = (S)-2-amino-3-(p-tolyl)propanoic acid
  SITE 2: note = (S)-cyclohexylglycine
  SITE 5: note = Amidated lysine
FGARK

SEQ ID NO: 332 - protein, 5 aa, synthetic construct
  SITE 1: note = (S)-2-amino-3-(4-(trifluoromethyl)phenyl)propanoic acid
  SITE 2: note = (S)-cyclohexylglycine
  SITE 5: note = Amidated lysine
FGARK

SEQ ID NO: 333 - protein, 5 aa, synthetic construct
  SITE 1: note = (S)-2-amino-3-(2,4-difluorophenyl)propanoic acid
  SITE 2: note = (S)-cyclohexylglycine
  SITE 5: note = Amidated lysine
FGARK

SEQ ID NO: 334 - protein, 5 aa, synthetic construct
  SITE 1: note = (S)-2-amino-3-(2,5-dichlorophenyl)propanoic acid
  SITE 2: note = (S)-cyclohexylglycine
  SITE 5: note = Amidated lysine
FGARK

SEQ ID NO: 335 - protein, 5 aa, synthetic construct
  SITE 1: note = (S)-2-amino-3-(4-vinylphenyl)propanoic acid
  SITE 2: note = (S)-cyclohexylglycine
  SITE 5: note = Amidated lysine
FGARK

SEQ ID NO: 336 - protein, 5 aa, synthetic construct
  SITE 1: note = (S)-2-amino-3-(3,4,5-trifluorophenyl)propanoic acid
  SITE 2: note = (S)-cyclohexylglycine
  SITE 5: note = Amidated lysine
FGARK

SEQ ID NO: 337 - protein, 5 aa, synthetic construct
  SITE 1: note = (S)-2-amino-3-cyclopentylpropanoic acid
  SITE 2: note = (S)-cyclohexylglycine
  SITE 5: note = Amidated lysine
AGARK

SEQ ID NO: 338 - protein, 5 aa, synthetic construct
  SITE 1: note = (S)-2-amino-3-(3,4-bis(trifluoromethyl)phenyl)propa noic acid
  SITE 2: note = (S)-cyclohexylglycine
  SITE 5: note = Amidated lysine
FGARK

SEQ ID NO: 339 - protein, 5 aa, synthetic construct
  SITE 1: note = (S)-cyclohexylalanine
  SITE 2: note = (S)-2-amino-2-((S)-2,3-dihydro-1H-inden-1-yl)acetic acid
  SITE 5: note = Amidated lysine
AXARK

SEQ ID NO: 340 - protein, 5 aa, synthetic construct
  SITE 1: note = (S)-cyclohexylalanine
  SITE 2: note = (S)-2-amino-2-((S)-1,2,3,4-tetrahydronaphthalen-1-y l)acetic acid
  SITE 5: note = Amidated lysine
AXARK

SEQ ID NO: 341 - protein, 5 aa, synthetic construct
  SITE 1: note = (S)-2-amino-3-(perfluorophenyl)propanoic acid
  SITE 2: note = (S)-cyclohexylglycine
  SITE 5: note = Amidated lysine
FGARK

SEQ ID NO: 342 - protein, 5 aa, synthetic construct
  SITE 1: note = (S)-2-amino-3-(2,3-dihydrobenzofuran-6-yl)propanoic acid
  SITE 2: note = (S)-cyclohexylglycine
  SITE 5: note = Amidated lysine
FGARK

SEQ ID NO: 343 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 344 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 345 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 346 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 347 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 348 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 349 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 350 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 351 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 352 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 353 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 354 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 355 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 356 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 357 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 358 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 359 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 360 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 361 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 362 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 363 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 364 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 365 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 366 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 367 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 368 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 369 - protein, 5 aa, synthetic construct
  SITE 1: note = (S)-cyclohexylalanine
  SITE 2: note = (S)-cyclohexylglycine
  SITE 5: note = Amidated lysine
AGARK

SEQ ID NO: 370 - protein, 5 aa, synthetic construct
  SITE 1: note = (S)-cyclohexylalanine
  SITE 2: note = (S)-cyclohexylglycine
  SITE 4: note = (S)-2-amino-3-(4-((R)-2,6-dioxohexahydropyrimidine- 4-carboxamido)phenyl)propanoic acid
  SITE 5: note = Amidated lysine
AGAXK

SEQ ID NO: 371 - protein, 5 aa, synthetic construct
  SITE 1: note = (S)-cyclohexylalanine
  SITE 2: note = (S)-cyclohexylglycine
  SITE 5: note = Ornithine1,4,7,10-tetraazacyclododecane-1,4,7,10-te traacetic acid
  SITE 5: note = Amidated lysine
AGARX

SEQ ID NO: 372 - protein, 5 aa, synthetic construct
  SITE 1: note = (S)-cyclohexylalanine
  SITE 2: note = (S)-cyclohexylglycine
  SITE 4: note = (Z)-N-omega'-methyl-L-arginine
  SITE 5: note = Lysine1,4,7,10-tetraazacyclododecane-1,4,7,10-tetra acetic acid
  SITE 5: note = Amidated lysine
AGARK

SEQ ID NO: 373 - protein, 5 aa, synthetic construct
  SITE 1: note = (S)-cyclohexylalanine
  SITE 2: note = (S)-cyclohexylglycine
  SITE 4: note = (S)-2-amino-2-(piperidin-4-yl)acetic acid
  SITE 5: note = Lysine1,4,7,10-tetraazacyclododecane-1,4,7,10-tetra acetic acid
  SITE 5: note = Amidated lysine
AGAGK

SEQ ID NO: 374 - protein, 5 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = Lysine1,4,7,10-tetraazacyclododecane-1,4,7,10-tetra acetic acid
  SITE 5: note = Amidated lysine
AGAXK

SEQ ID NO: 375 - protein, 5 aa, synthetic construct
  SITE 1: note = (S)-cyclohexylalanine
  SITE 2: note = (S)-cyclohexylglycine
  SITE 4: note = 4-aminopiperidine-4-carboxylic acid
  SITE 5: note = Amidated Lysine(1,4,7,10-tetraazacyclododecane-1,4,7,10-tetraacetic acid)
AGAXK

SEQ ID NO: 376 - protein, 5 aa, synthetic construct
  SITE 1: note = (S)-cyclohexylalanine
  SITE 2: note = (S)-cyclohexylglycine
  SITE 4: note = 4-amino-1-carbamimidoylpiperidine-4-carboxylic acid
  SITE 5: note = Amidated Lysine(1,4,7,10-tetraazacyclododecane-1,4,7,10-tetraacetic acid)
AGAXK

SEQ ID NO: 377 - protein, 5 aa, synthetic construct
  SITE 1: note = (S)-cyclohexylalanine
  SITE 2: note = (S)-cyclohexylglycine
  SITE 4: note = 4-amino-1-(methyl-D-alanyl)piperidine-4-carboxylic acid
  SITE 5: note = Amidated Lysine(1,4,7,10-tetraazacyclododecane-1,4,7,10-tetraacetic acid)
AGAXK

SEQ ID NO: 378 - protein, 5 aa, synthetic construct
  SITE 1: note = (S)-cyclohexylalanine
  SITE 2: note = (S)-cyclohexylglycine
  SITE 3: note = N-methyl-Alanine
  SITE 5: note = Amidated Lysine(1,4,7,10-tetraazacyclododecane-1,4,7,10-tetraacetic acid)
AGARK

SEQ ID NO: 379 - protein, 5 aa, synthetic construct
  SITE 1: note = (S)-cyclohexylalanine
  SITE 2: note = (S)-cyclohexylglycine
  SITE 4: note = N-methyl-Arginine
  SITE 5: note = Amidated Lysine(1,4,7,10-tetraazacyclododecane-1,4,7,10-tetraacetic acid)
AGARK

SEQ ID NO: 380 - protein, 6 aa, synthetic construct
  SITE 1: note = (S)-cyclohexylalanine
  SITE 2: note = (S)-cyclohexylglycine
  SITE 5: note = Amidated D-Lysine(1,4,7,10-tetraazacyclododecane-1, 4,7,10-tetraacetic acid)
AGARDK

SEQ ID NO: 381 - protein, 6 aa, synthetic construct
  SITE 1: note = (S)-cyclohexylalanine
  SITE 2: note = (S)-cyclohexylglycine
  SITE 4: note = D-arginine
  SITE 5: note = Amidated Lysine(1,4,7,10-tetraazacyclododecane-1,4,7,10-tetraacetic acid)
AGADRK

SEQ ID NO: 382 - protein, 6 aa, synthetic construct
  SITE 1: note = (S)-cyclohexylalanine
  SITE 2: note = (S)-cyclohexylglycine
  SITE 3: note = D-Alanine
  SITE 5: note = Amidated Lysine(1,4,7,10-tetraazacyclododecane-1,4,7,10-tetraacetic acid)
AGDARK

SEQ ID NO: 383 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 384 - protein, 6 aa, synthetic construct
  SITE 1: note = (S)-cyclohexylalanine
  SITE 2: note = (S)-cyclohexylglycine
  SITE 5: note = (S)-2-amino-3-(3-cyclopropylphenyl)propanoic acid
  SITE 6: note = Amidated Lysine(1,4,7,10-tetraazacyclododecane-1,4,7,10-tetraacetic acid)
AGARXK

SEQ ID NO: 385 - protein, 6 aa, synthetic construct
  SITE 1: note = (S)-cyclohexylalanine
  SITE 2: note = (S)-cyclohexylglycine
  SITE 5: note = 4-amino-1-((3-fluoro-4-methylbenzyl)glycyl)piperidi ne-4-carboxylic acid
  SITE 6: note = Amidated Lysine(1,4,7,10-tetraazacyclododecane-1,4,7,10-tetraacetic acid)
AGARXK

SEQ ID NO: 386 - protein, 7 aa, synthetic construct
  SITE 1: note = (S)-cyclohexylalanine
  SITE 2: note = (S)-cyclohexylglycine
  SITE 6: note = 4-amino-1-(methylglycyl)piperidine-4-carboxylic acid
  SITE 7: note = Amidated Lysine(1,4,7,10-tetraazacyclododecane-1,4,7,10-tetraacetic acid)
AGARAXK

SEQ ID NO: 387-388 - skipped (2 entries)

SEQ ID NO: 389 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Lysine(1,4,7,10-tetraazacyclododecane-1,4,7,10-tetraacetic acid)
FGAFK

SEQ ID NO: 390 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = 2-(2-(2-aminoethoxy)ethoxy)acetic acid
  SITE 6: note = Amidated D-Lysine(1,4,7,10-tetraazacyclododecane-1, 4,7,10-tetraacetic acid)
FGAFXK

SEQ ID NO: 391 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = 2-(2-(2-aminoethoxy)ethoxy)acetic acid
  SITE 6: note = Amidated D-Lysine(indium(III) (R)-2,2',2''-(10-(2-( (5,6-diamino-6-oxohexyl)amino)-2-oxoethyl)-1,4,7,10-tetraa zacyclododecane-1,4,7-triyl)triacetate)
FGAFXK

SEQ ID NO: 392 - protein, 5 aa, synthetic construct
  SITE 1: note = (S)-cyclohexylalanine
  SITE 2: note = (S)-cyclohexylglycine
  SITE 3: note = (S)-3-([1,1'-biphenyl]-4-yl)-2-aminopropanoic acid
  SITE 4: note = 2-amino-5-((2-aminopyridin-4-yl)amino)pentanoic acid
  SITE 5: note = Amidated Ornithine(1,4,7,10-tetraazacyclododecane-1 ,4,7,10-tetraacetic acid)
AGFKX

SEQ ID NO: 393-394 - skipped (2 entries)

SEQ ID NO: 395 - protein, 5 aa, synthetic construct
  SITE 1: note = (S)-cyclohexylalanine
  SITE 2: note = (S)-2-amino-3-ethylpentanoic acid
  SITE 5: note = Amidated Lysine(1,4,7,10-tetraazacyclododecane-1,4,7,10-tetraacetic acid)
AXARK

SEQ ID NO: 396 - protein, 5 aa, synthetic construct
  SITE 1: note = (S)-cyclohexylalanine
  SITE 2: note = (S)-cyclohexylglycine
  SITE 4: note = (S)-2,5-diaminopentanoic acid
  SITE 5: note = Amidated Lysine(1,4,7,10-tetraazacyclododecane-1,4,7,10-tetraacetic acid)
AGAXK

SEQ ID NO: 397 - protein, 5 aa, synthetic construct
  SITE 1: note = (S)-cyclohexylalanine
  SITE 2: note = (S)-cyclohexylglycine
  SITE 4: note = (S)-2,3-diaminopropanoic acid
  SITE 5: note = Amidated Lysine(1,4,7,10-tetraazacyclododecane-1,4,7,10-tetraacetic acid)
AGAXK

SEQ ID NO: 398 - protein, 5 aa, synthetic construct
  SITE 1: note = (S)-cyclohexylalanine
  SITE 2: note = (S)-2-amino-3-ethylpentanoic acid
  SITE 2: note = (S)-2-amino-2-(piperidin-4-yl)acetic acid
  SITE 5: note = Amidated Lysine(1,4,7,10-tetraazacyclododecane-1,4,7,10-tetraacetic acid)
AXAGK

SEQ ID NO: 399 - protein, 6 aa, synthetic construct
  SITE 1: note = (S)-cyclohexylalanine
  SITE 2: note = (S)-cyclohexylglycine
  SITE 4: note = (S)-2-amino-2-(piperidin-4-yl)acetic acid
  SITE 5: note = 2-(2-(2-aminoethoxy)ethoxy)acetic acid
  SITE 6: note = Amidated Lysine(1,4,7,10-tetraazacyclododecane-1,4,7,10-tetraacetic acid)
AGAGXK

SEQ ID NO: 400 - protein, 5 aa, synthetic construct
  SITE 1: note = (S)-cyclohexylalanine
  SITE 2: note = (S)-cyclohexylglycine
  SITE 4: note = (S)-2-amino-3-(pyridin-4-yl)propanoic acid
  SITE 5: note = Amidated Lysine(1,4,7,10-tetraazacyclododecane-1,4,7,10-tetraacetic acid)
AGAXK

SEQ ID NO: 401 - protein, 5 aa, synthetic construct
  SITE 1: note = (S)-cyclohexylalanine
  SITE 2: note = (S)-cyclohexylglycine
  SITE 4: note = (S)-2-amino-3-(4-aminophenyl)propanoic acid
  SITE 5: note = Amidated Lysine(1,4,7,10-tetraazacyclododecane-1,4,7,10-tetraacetic acid)
AGAFK

SEQ ID NO: 402 - protein, 5 aa, synthetic construct
  SITE 1: note = (S)-cyclohexylalanine
  SITE 2: note = (S)-cyclohexylglycine
  SITE 3: note = 2-amino-2-methylpropanoic acid
  SITE 4: note = (S)-2-amino-2-(piperidin-4-yl)acetic acid
  SITE 5: note = Amidated Lysine(1,4,7,10-tetraazacyclododecane-1,4,7,10-tetraacetic acid)
AGAGK

SEQ ID NO: 403 - protein, 5 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(piperidin-4-yl)acetic acid
  SITE 5: note = Amidated Lysine(1,4,7,10-tetraazacyclododecane-1,4,7,10-tetraacetic acid)
FGAGK

SEQ ID NO: 404 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Lysine(1,4,7,10-tetraazacyclododecane-1,4,7,10-tetraacetic acid)
FGAFEK

SEQ ID NO: 405 - protein, 5 aa, synthetic construct
  SITE 1: note = (S)-cyclohexylalanine
  SITE 2: note = (S)-cyclohexylglycine
  SITE 4: note = 2-amino-5-((2-aminopyridin-4-yl)amino)pentanoic acid
  SITE 5: note = Amidated Ornithine(2,2',2''-(10-(2-((1-(carboxymeth yl)piperidin-4-yl)amino)-2-oxoethyl)-1,4,7,10-tetraazacycl ododecane-1,4,7-triyl)triacetic acid)
AGAKX

SEQ ID NO: 406 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = 2-(2-(2-aminoethoxy)ethoxy)acetic acid
  SITE 6: note = Amidated D-Lysine-2,2',2''-(10-(2-((((1r,4r)-4-carb oxycyclohexyl)methyl)amino)-2-oxoethyl)-1,4,7,10-tetraazac yclododecane-1,4,7-triyl)triacetic acid
FGAXXK

SEQ ID NO: 407 - protein, 5 aa, synthetic construct
  SITE 1: note = (S)-cyclohexylalanine
  SITE 2: note = (S)-2-amino-3-ethylpentanoic acid
  SITE 5: note = Amidated Lysine(1,4,7,10-tetraazacyclododecane-1,4,7,10-tetraacetic acid)
AXARK

SEQ ID NO: 408 - protein, 5 aa, synthetic construct
  SITE 1: note = (S)-cyclohexylalanine
  SITE 2: note = (S)-cyclohexylglycine
  SITE 4: note = (S)-2,5-diaminopentanoic acid
  SITE 5: note = Amidated Lysine(1,4,7,10-tetraazacyclododecane-1,4,7,10-tetraacetic acid)
AGAXK

SEQ ID NO: 409 - protein, 5 aa, synthetic construct
  SITE 1: note = (S)-cyclohexylalanine
  SITE 2: note = (S)-2-amino-3-ethylpentanoic acid
  SITE 4: note = (S)-2-amino-2-(piperidin-4-yl)acetic acid
  SITE 5: note = Amidated Lysine(1,4,7,10-tetraazacyclododecane-1,4,7,10-tetraacetic acid)
AXAGK

SEQ ID NO: 410 - protein, 6 aa, synthetic construct
  SITE 1: note = (S)-cyclohexylalanine
  SITE 2: note = (S)-cyclohexylglycine
  SITE 4: note = (S)-2-amino-2-(piperidin-4-yl)acetic acid
  SITE 5: note = 3-(2-(2-aminoethoxy)ethoxy)propanoic acid
  SITE 5: note = Amidated Lysine(1,4,7,10-tetraazacyclododecane-1,4,7,10-tetraacetic acid)
AGAGXK

SEQ ID NO: 411 - protein, 5 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(piperidin-4-yl)acetic acid
  SITE 5: note = Amidated Lysine(1,4,7,10-tetraazacyclododecane-1,4,7,10-tetraacetic acid)
FGAGK

SEQ ID NO: 412 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Lysine(2-(2-(2-aminoethoxy)ethoxy)acetic acid-1,4,7,10-tetraazacyclododecane-1,4,7,10-tetraacetic acid)
FGAFK

SEQ ID NO: 413 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Lysine(2-(2-(2-aminoethoxy)ethoxy)acetic acid-1,4,7,10-tetraazacyclododecane-1,4,7,10-tetraacetic acid)
FGAFK

SEQ ID NO: 414 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Lysine(2-(2-(2-aminoethoxy)ethoxy)acetic acid-1,4,7,10-tetraazacyclododecane-1,4,7,10-tetraacetic acid)
AGAFK

SEQ ID NO: 415 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 6: note = Amidated Lysine(1,4,7,10-tetraazacyclododecane-1,4,7,10-tetraacetic acid)
AGAFXK

SEQ ID NO: 416 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 6: note = Amidated Lysine(1,4,7,10-tetraazacyclododecane-1,4,7,10-tetraacetic acid)
AGAFEK

SEQ ID NO: 417 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Lysine(2,2',2''-(10-(2-((2-(2-(carboxymeth oxy)ethoxy)ethyl)amino)-2-oxoethyl)-1,4,7,10-tetraazacyclo dodecane-1,4,7-triyl)triacetic acid)
AGAFK

SEQ ID NO: 418 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Lysine(2,2',2''-(10-(2-((5-carboxypentyl)a mino)-2-oxoethyl)-1,4,7,10-tetraazacyclododecane-1,4,7-tri yl)triacetic acid)
AGAFK

SEQ ID NO: 419 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 6: note = Amidated Lysine(2-(2-(2-aminoethoxy)ethoxy)acetic acid-1,4,7,10-tetraazacyclododecane-1,4,7,10-tetraacetic acid)
AGAFKK

SEQ ID NO: 420 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = N6-palmitoyl-L-lysine
  SITE 6: note = Amidated Lysine(1,4,7,10-tetraazacyclododecane-1,4,7,10-tetraacetic acid)
AGAFKK

SEQ ID NO: 421 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = (S)-16-((5-amino-5-carboxypentyl)amino)-16-oxohexad ecanoic acid
  SITE 6: note = Amidated Lysine(2-(2-(2-aminoethoxy)ethoxy)acetic acid-1,4,7,10-tetraazacyclododecane-1,4,7,10-tetraacetic acid)
AGAFKK

SEQ ID NO: 422 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = (S)-2-(16-((5-amino-5-carboxypentyl)amino)-16-oxohe xadecanamido)-N,N,N-trimethylethan-1-aminium
  SITE 6: note = Amidated Lysine(2-(2-(2-aminoethoxy)ethoxy)acetic acid-1,4,7,10-tetraazacyclododecane-1,4,7,10-tetraacetic acid)
AGAFKK

SEQ ID NO: 423 - protein, 6 aa, synthetic construct
  SITE 1: note = (S)-cyclohexylalanine
  SITE 2: note = (S)-cyclohexylglycine
  SITE 4: note = 2-amino-5-((2-aminopyridin-4-yl)amino)pentanoic acid
  SITE 5: note = (S)-2-amino-3-(4-nitrophenyl)propanoic acid
  SITE 6: note = Amidated Lysine(1,4,7,10-tetraazacyclododecane-1,4,7,10-tetraacetic acid)
AGAKFK

SEQ ID NO: 424 - protein, 6 aa, synthetic construct
  SITE 1: note = (S)-cyclohexylalanine
  SITE 2: note = (S)-cyclohexylglycine
  SITE 4: note = 2-amino-5-((2-aminopyridin-4-yl)amino)pentanoic acid
  SITE 5: note = (S)-2-amino-3-(4-iodophenyl)propanoic acid
  SITE 6: note = Amidated Lysine(1,4,7,10-tetraazacyclododecane-1,4,7,10-tetraacetic acid)
AGAKFK

SEQ ID NO: 425 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Ornithine(1,4,7,10-tetraazacyclododecane-1 ,4,7,10-tetraacetic acid)
AGAKX

SEQ ID NO: 426 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Ornithine(1,4,7,10-tetraazacyclododecane-1 ,4,7,10-tetraacetic acid)
FGAKX

SEQ ID NO: 427 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Ornithine
  SITE 6: note = Amidated Lysine(1,4,7,10-tetraazacyclododecane-1,4,7,10-tetraacetic acid)
AGAFXK

SEQ ID NO: 428 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = (S)-2,3-diaminopropanoic acid
  SITE 6: note = Amidated Lysine(1,4,7,10-tetraazacyclododecane-1,4,7,10-tetraacetic acid)
AGAFXK

SEQ ID NO: 429 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 5: note = Amidated Lysine(1,4,7,10-tetraazacyclododecane-1,4,7,10-tetraacetic acid)
AGARK

SEQ ID NO: 430 - protein, 5 aa, synthetic construct
  SITE 1: note = (S)-cyclohexylalanine
  SITE 2: note = (S)-cyclohexylglycine
  SITE 4: note = (S)-2-amino-3-(6-aminopyridin-3-yl)propanoic acid
  SITE 5: note = Amidated Lysine(1,4,7,10-tetraazacyclododecane-1,4,7,10-tetraacetic acid)
AGAXK

SEQ ID NO: 431 - protein, 5 aa, synthetic construct
  SITE 1: note = (S)-cyclohexylalanine
  SITE 2: note = (S)-cyclohexylglycine
  SITE 4: note = N4,N4-dimethyl-L-asparagine
  SITE 5: note = Amidated Lysine(1,4,7,10-tetraazacyclododecane-1,4,7,10-tetraacetic acid)
AGANK

SEQ ID NO: 432 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 433 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 434 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 435 - protein, 6 aa, synthetic construct
  SITE 1: note = (S)-cyclohexylalanine
  SITE 2: note = (S)-cyclohexylglycine
  SITE 4: note = (S)-2-amino-5-((2-aminopyridin-4-yl)amino)pentanoic acid
  SITE 5: note = (S)-2-amino-3-(4-iodophenyl)propanoic acid
  SITE 6: note = Amidated Lysine(1,4,7,10-tetraazacyclododecane-1,4,7,10-tetraacetic acid)
AGAXFK

SEQ ID NO: 436 - protein, 6 aa, synthetic construct
  SITE 1: note = (S)-cyclohexylalanine
  SITE 2: note = (S)-cyclohexylglycine
  SITE 4: note = (S)-2-amino-5-((2-aminopyridin-4-yl)amino)pentanoic acid
  SITE 5: note = (S)-2-amino-3-(4-nitrophenyl)propanoic acid
  SITE 6: note = Amidated Lysine(1,4,7,10-tetraazacyclododecane-1,4,7,10-tetraacetic acid)
AGAXFK

SEQ ID NO: 437 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-5-((2-aminopyridin-4-yl)amino)pentanoic acid
  SITE 5: note = (S)-2-amino-3-(4-nitrophenyl)propanoic acid
  SITE 6: note = Amidated Lysine(1,4,7,10-tetraazacyclododecane-1,4,7,10-tetraacetic acid)
AGAXFK

SEQ ID NO: 438 - protein, 6 aa, synthetic construct
  SITE 1: note = (S)-cyclohexylalanine
  SITE 2: note = (S)-cyclohexylglycine
  SITE 4: note = (S)-2-amino-5-((2-aminopyridin-4-yl)amino)pentanoic acid
  SITE 5: note = (S)-2-amino-3-(4-nitrophenyl)propanoic acid
  SITE 6: note = Amidated Lysine(1,4,7,10-tetraazacyclododecane-1,4,7,10-tetraacetic acid)
AGAXFK

SEQ ID NO: 439 - protein, 5 aa, synthetic construct
  SITE 1: note = (S)-cyclohexylalanine
  SITE 2: note = (S)-cyclohexylglycine
  SITE 4: note = N6-carbamimidoyl-L-lysine
  SITE 5: note = Amidated Lysine(1,4,7,10-tetraazacyclododecane-1,4,7,10-tetraacetic acid)
AGAKK

SEQ ID NO: 440 - protein, 5 aa, synthetic construct
  SITE 1: note = (S)-cyclohexylalanine
  SITE 2: note = (S)-cyclohexylglycine
  SITE 4: note = (S)-2-amino-3-(2,4-difluorophenyl)propanoic acid
  SITE 5: note = Amidated Lysine(1,4,7,10-tetraazacyclododecane-1,4,7,10-tetraacetic acid)
AGAFK

SEQ ID NO: 441 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 442 - protein, 5 aa, synthetic construct
  SITE 1: note = (S)-cyclohexylalanine
  SITE 2: note = (S)-cyclohexylglycine
  SITE 5: note = Ornithine(1,4,7,10-tetraazacyclododecane-1,4,7,10-t etraacetic acid)
AGARX

SEQ ID NO: 443 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 444 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 445 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 446 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 447 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 448 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 449 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 450 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 451 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 452 - protein, 5 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Ornithine(1,4,7,10-tetraazacyclododecane-1 ,4,7,10-tetraacetic acid)
AGARK

SEQ ID NO: 453 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Amidated Ornithine(1,4,7,10-tetraazacyclododecane-1 ,4,7,10-tetraacetic acid)
AGARX

SEQ ID NO: 454 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Ornithine
  SITE 6: note = Amidated Lysine(1,4,7,10-tetraazacyclododecane-1,4,7,10-tetraacetic acid)
AGAFXK

SEQ ID NO: 455 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = (S)-2,3-diaminopropanoic acid
  SITE 6: note = Amidated Lysine(1,4,7,10-tetraazacyclododecane-1,4,7,10-tetraacetic acid)
AGAFXK

SEQ ID NO: 456 - protein, 5 aa, synthetic construct
  SITE 1: note = (S)-cyclohexylalanine
  SITE 2: note = (S)-cyclohexylglycine
  SITE 4: note = (S)-2-amino-5-oxo-5-(piperazin-1-yl)pentanoic acid
  SITE 5: note = Amidated Ornithine(1,4,7,10-tetraazacyclododecane-1 ,4,7,10-tetraacetic acid)
AGAQX

SEQ ID NO: 457-459 - skipped (3 entries)

SEQ ID NO: 460 - protein, 5 aa, synthetic construct
  SITE 1: note = (S)-cyclohexylalanine
  SITE 2: note = (S)-cyclohexylglycine
  SITE 4: note = N6-nicotinoyl-L-lysine
  SITE 5: note = Amidated Ornithine(1,4,7,10-tetraazacyclododecane-1 ,4,7,10-tetraacetic acid)
AGAKX

SEQ ID NO: 461 - protein, 6 aa, synthetic construct
  SITE 1: note = (S)-cyclohexylalanine
  SITE 2: note = (S)-cyclohexylglycine
  SITE 5: note = (S)-7-hydroxy-1,2,3,4-tetrahydroisoquinoline-3-carb oxylic acid
  SITE 6: note = Amidated Lysine(1,4,7,10-tetraazacyclododecane-1,4,7,10-tetraacetic acid)
AGARXK

SEQ ID NO: 462 - protein, 6 aa, synthetic construct
  SITE 1: note = (S)-cyclohexylalanine
  SITE 2: note = (S)-cyclohexylglycine
  SITE 4: note = (S)-2-amino-5-((2-aminopyridin-4-yl)amino)pentanoic acid
  SITE 5: note = (S)-7-hydroxy-1,2,3,4-tetrahydroisoquinoline-3-carb oxylic acid
  SITE 6: note = Amidated Lysine(1,4,7,10-tetraazacyclododecane-1,4,7,10-tetraacetic acid)
AGAXXK

SEQ ID NO: 463 - skipped

SEQ ID NO: 464 - protein, 5 aa, synthetic construct
  SITE 1: note = (S)-cyclohexylalanine
  SITE 2: note = (S)-cyclohexylglycine
  SITE 4: note = N6-(1-benzylpiperidine-4-carbonyl)-L-lysine
  SITE 5: note = Amidated Ornithine(1,4,7,10-tetraazacyclododecane-1 ,4,7,10-tetraacetic acid)
AGAKX

SEQ ID NO: 465 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 6: note = Amidated Lysine(1,4,7,10-tetraazacyclododecane-1,4,7,10-tetraacetic acid)
AGAFKK

SEQ ID NO: 466 - protein, 7 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 6: note = 3-(2-(2-aminoethoxy)ethoxy)propanoic acid
  SITE 7: note = Amidated Lysine(1,4,7,10-tetraazacyclododecane-1,4,7,10-tetraacetic acid)
AGAFKXK

SEQ ID NO: 467 - protein, 7 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = N6-palmitoyl-L-lysine
  SITE 6: note = 3-(2-(2-aminoethoxy)ethoxy)propanoic acid
  SITE 6: note = Amidated Lysine(1,4,7,10-tetraazacyclododecane-1,4,7,10-tetraacetic acid)
AGAFKXK

SEQ ID NO: 468 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = 4-amino-1-((3-fluoro-4-methylbenzyl)glycyl)piperidi ne-4-carboxylic acid
  SITE 6: note = Amidated Lysine(1,4,7,10-tetraazacyclododecane-1,4,7,10-tetraacetic acid)
AGAXXK

SEQ ID NO: 469 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = (S)-2-amino-3-(3-cyclopropylphenyl)propanoic acid
  SITE 6: note = Amidated Lysine(1,4,7,10-tetraazacyclododecane-1,4,7,10-tetraacetic acid)
AGAXXK

SEQ ID NO: 470 - protein, 7 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 6: note = 4-amino-1-(methylglycyl)piperidine-4-carboxylic acid
  SITE 6: note = Amidated Lysine(1,4,7,10-tetraazacyclododecane-1,4,7,10-tetraacetic acid)
AGAXAXK

SEQ ID NO: 471 - protein, 7 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 6: note = 4-amino-1-(methylglycyl)piperidine-4-carboxylic acid
  SITE 6: note = Amidated Lysine(1,4,7,10-tetraazacyclododecane-1,4,7,10-tetraacetic acid)
AGAXAXK

SEQ ID NO: 472 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 6: note = 4-amino-1-((3-fluoro-4-methylbenzyl)glycyl)piperidi ne-4-carboxylic acid
  SITE 6: note = Amidated Lysine(1,4,7,10-tetraazacyclododecane-1,4,7,10-tetraacetic acid)
AGAXXK

SEQ ID NO: 473 - protein, 7 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 6: note = (S)-2-amino-3-(4-fluorobenzamido)propanoic acid
  SITE 7: note = Amidated Lysine(1,4,7,10-tetraazacyclododecane-1,4,7,10-tetraacetic acid)
AGAXAXK

SEQ ID NO: 474 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
FGAXK

SEQ ID NO: 475 - skipped

SEQ ID NO: 476 - protein, 5 aa, synthetic construct
  SITE 1: note = (S)-2-amino-2-(2,3-dihydro-1H-inden-2-yl)acetic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
GGAXK

SEQ ID NO: 477 - protein, 5 aa, synthetic construct
  SITE 1: note = (S)-2-amino-3-cycloheptylpropanoic acid
  SITE 2: note = (S)-2-amino-2-(4,4-difluorocyclohexyl)acetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 478 - protein, 5 aa, synthetic construct
  SITE 1: note = (S)-2-amino-3-cycloheptylpropanoic acid
  SITE 2: note = (S)-2-amino-2-cycloheptylacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 479 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 480 - protein, 5 aa, synthetic construct
  SITE 1: note = (S)-2-amino-3-cycloheptylpropanoic acid
  SITE 2: note = (S)-2-amino-2-cycloheptylacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 481 - protein, 5 aa, synthetic construct
  SITE 1: note = (S)-2-amino-3-cycloheptylpropanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 482 - protein, 5 aa, synthetic construct
  SITE 1: note = (S)-2-amino-3-cycloheptylpropanoic acid
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 483 - protein, 5 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 484 - protein, 5 aa, synthetic construct
  SITE 1: note = (S)-2-amino-3-cycloheptylpropanoic acid
  SITE 2: note = (S)-cyclohexylglycine
  SITE 4: note = (S)-2-amino-3-(6-aminopyridin-3-yl)propanoic acid
  SITE 5: note = Amidated Lysine(1,4,7,10-tetraazacyclododecane-1,4,7,10-tetraacetic acid)
AGAXK

SEQ ID NO: 485 - protein, 5 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-3-(6-aminopyridin-3-yl)propanoic acid
  SITE 5: note = Amidated Lysine(1,4,7,10-tetraazacyclododecane-1,4,7,10-tetraacetic acid)
AGAXK

SEQ ID NO: 486 - protein, 5 aa, synthetic construct
  SITE 1: note = (S)-2-amino-3-cycloheptylpropanoic acid
  SITE 2: note = (S)-2-amino-2-cycloheptylacetic acid
  SITE 4: note = (S)-2-amino-3-(6-aminopyridin-3-yl)propanoic acid
  SITE 4: note = Amidated Lysine(1,4,7,10-tetraazacyclododecane-1,4,7,10-tetraacetic acid)
AGAXK

SEQ ID NO: 487 - protein, 5 aa, synthetic construct
  SITE 1: note = (S)-2-amino-3-cycloheptylpropanoic acid
  SITE 2: note = (S)-2-amino-2-cycloheptylacetic acid
  SITE 4: note = (S)-2-amino-3-(6-aminopyridin-3-yl)propanoic acid
  SITE 5: note = Amidated Lysine(1,4,7,10-tetraazacyclododecane-1,4,7,10-tetraacetic acid)
AGAXK

SEQ ID NO: 488 - protein, 5 aa, synthetic construct
  SITE 1: note = (S)-2-amino-3-cycloheptylpropanoic acid
  SITE 2: note = (S)-2-amino-2-cycloheptylacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 489 - protein, 5 aa, synthetic construct
  SITE 1: note = (S)-2-amino-3-cycloheptylpropanoic acid
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 490 - protein, 5 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 491 - protein, 5 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 492 - protein, 5 aa, synthetic construct
  SITE 1: note = (S)-cyclohexylalanine
  SITE 2: note = (S)-cyclohexylglycine
  SITE 4: note = 2-amino-5-((2-aminopyridin-4-yl)amino)pentanoic acid
  SITE 5: note = Amidated Lysine(1,4,7,10-tetraazacyclododecane-1,4,7,10-tetraacetic acid)
AGAXK

SEQ ID NO: 493 - protein, 5 aa, synthetic construct
  SITE 1: note = (S)-cyclohexylalanine
  SITE 2: note = (S)-cyclohexylglycine
  SITE 4: note = 2-amino-5-((2-aminopyridin-4-yl)amino)pentanoic acid
  SITE 5: note = Amidated Lysine(1,4,7,10-tetraazacyclododecane-1,4,7,10-tetraacetic acid)
AGAXK

SEQ ID NO: 494 - protein, 5 aa, synthetic construct
  SITE 1: note = (S)-cyclohexylalanine
  SITE 2: note = (S)-cyclohexylglycine
  SITE 4: note = 2-amino-5-((2-aminopyridin-4-yl)amino)pentanoic acid
  SITE 5: note = Amidated Lysine(1,4,7,10-tetraazacyclododecane-1,4,7,10-tetraacetic acid)
AGAXK

SEQ ID NO: 495 - protein, 5 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = 2-amino-5-((2-aminopyridin-4-yl)amino)pentanoic acid
  SITE 5: note = Amidated Lysine(1,4,7,10-tetraazacyclododecane-1,4,7,10-tetraacetic acid)
AGAXK

SEQ ID NO: 496 - protein, 5 aa, synthetic construct
  SITE 1: note = (S)-2-amino-3-cycloheptylpropanoic acid
  SITE 2: note = (S)-2-amino-2-cycloheptylacetic acid
  SITE 4: note = 2-amino-5-((2-aminopyridin-4-yl)amino)pentanoic acid
  SITE 5: note = Amidated Lysine(1,4,7,10-tetraazacyclododecane-1,4,7,10-tetraacetic acid)
AGAXK

SEQ ID NO: 497 - protein, 5 aa, synthetic construct
  SITE 1: note = (S)-2-amino-3-cyclooctylpropanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 498 - protein, 5 aa, synthetic construct
  SITE 1: note = (S)-2-amino-3-cycloheptylpropanoic acid
  SITE 2: note = (2S)-2-((1r,3R,5S)-adamantan-1-yl)-2-aminoacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AGAXK

SEQ ID NO: 499 - protein, 6 aa, synthetic construct
  SITE 1: note = (S)-2-amino-3-cycloheptylpropanoic acid
  SITE 2: note = (S)-2-amino-2-cycloheptylacetic acid
  SITE 4: note = (S)-2-amino-2-(1-carbamimidoylpiperidin-4-yl)acetic acid
  SITE 5: note = Amidated Ornithine(1,4,7,10-tetraazacyclododecane-1 ,4,7,10-tetraacetic acid)
AGAXXK

SEQ ID NO: 500 - protein, 5 aa, synthetic construct
  SITE 1: note = (S)-2-amino-3-cycloheptylpropanoic acid
  SITE 2: note = (S)-cyclohexylglycine
  SITE 5: note = Amidated Lysine(1,4,7,10-tetraazacyclododecane-1,4,7,10-tetraacetic acid)
AGAFK

SEQ ID NO: 501 - protein, 5 aa, synthetic construct
  SITE 1: note = (S)-2-amino-3-cycloheptylpropanoic acid
  SITE 2: note = (S)-cyclohexylglycine
  SITE 4: note = (S)-cyclohexylalanine
  SITE 5: note = Amidated Lysine(1,4,7,10-tetraazacyclododecane-1,4,7,10-tetraacetic acid)
AGAAK

SEQ ID NO: 502 - protein, 5 aa, synthetic construct
  SITE 1: note = (S)-2-amino-3-cycloheptylpropanoic acid
  SITE 2: note = (S)-cyclohexylglycine
  SITE 4: note = (S)-2-amino-3-(4-fluorophenyl)propanoic acid
  SITE 5: note = Amidated Lysine(1,4,7,10-tetraazacyclododecane-1,4,7,10-tetraacetic acid)
AGAFK

SEQ ID NO: 503 - protein, 5 aa, synthetic construct
  SITE 1: note = (S)-2-amino-3-cycloheptylpropanoic acid
  SITE 2: note = (S)-cyclohexylglycine
  SITE 4: note = (S)-2-amino-2-phenylacetic acid
  SITE 5: note = Amidated Lysine(1,4,7,10-tetraazacyclododecane-1,4,7,10-tetraacetic acid)
AGAXK

SEQ ID NO: 504 - protein, 5 aa, synthetic construct
  SITE 1: note = (S)-2-amino-3-cycloheptylpropanoic acid
  SITE 2: note = (S)-cyclohexylglycine
  SITE 4: note = (S)-cyclohexylglycine
  SITE 5: note = Amidated Lysine(1,4,7,10-tetraazacyclododecane-1,4,7,10-tetraacetic acid)
AGAGK

SEQ ID NO: 505 - skipped

SEQ ID NO: 506 - protein, 7 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 6: note = 4-amino-1-(N-carbamimidoyl-N-methylglycyl)piperidin e-4-carboxylic acid
  SITE 7: note = Amidated Lysine(1,4,7,10-tetraazacyclododecane-1,4,7,10-tetraacetic acid)
AGAXAXK

SEQ ID NO: 507 - protein, 7 aa, synthetic construct
  SITE 1: note = (S)-cyclohexylalanine
  SITE 2: note = (S)-cyclohexylglycine
  SITE 4: note = (S)-2-amino-2-(piperidin-4-yl)acetic acid
  SITE 6: note = 4-amino-1-(methylglycyl)piperidine-4-carboxylic acid
  SITE 7: note = Amidated Lysine(1,4,7,10-tetraazacyclododecane-1,4,7,10-tetraacetic acid)
AGAXAXK

SEQ ID NO: 508 - protein, 7 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-2-(piperidin-4-yl)acetic acid
  SITE 6: note = 4-amino-1-(methylglycyl)piperidine-4-carboxylic acid
  SITE 7: note = Amidated Lysine(1,4,7,10-tetraazacyclododecane-1,4,7,10-tetraacetic acid)
AGAXAXK

SEQ ID NO: 509 - protein, 5 aa, synthetic construct
  SITE 1: note = (S)-2-amino-3-(3-cyclopropylphenyl)propanoic acid
  SITE 2: note = (S)-cyclohexylglycine
  SITE 5: note = Amidated Lysine(1,4,7,10-tetraazacyclododecane-1,4,7,10-tetraacetic acid)
FGARK

SEQ ID NO: 510 - protein, 5 aa, synthetic construct
  SITE 1: note = (S)-cyclohexylalanine
  SITE 2: note = (S)-cyclohexylglycine
  SITE 4: note = 2-amino-5-((2-aminopyridin-4-yl)amino)pentanoic acid
  SITE 5: note = Amidated Lysine(1,4,7,10-tetraazacyclododecane-1,4,7,10-tetraacetic acid)
AGAXK

SEQ ID NO: 511 - protein, 5 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
FGAXK

SEQ ID NO: 512 - protein, 5 aa, synthetic construct
  SITE 1: note = (S)-2-amino-3-(4-fluorophenyl)propanoic acid
  SITE 2: note = (S)-2-amino-2-cycloheptylacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
FGAXK

SEQ ID NO: 513 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
FGAXK

SEQ ID NO: 514 - protein, 5 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
FGAXK

SEQ ID NO: 515 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = (S)-2-amino-3-(4-fluorobenzamido)propanoic acid
  SITE 6: note = Amidated Lysine(1,4,7,10-tetraazacyclododecane-1,4,7,10-tetraacetic acid)
AGAXXK

SEQ ID NO: 516 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = (S)-2-amino-3-(4-fluorobenzamido)propanoic acid
  SITE 6: note = Amidated Lysine(1,4,7,10-tetraazacyclododecane-1,4,7,10-tetraacetic acid)
AGAXXK

SEQ ID NO: 517 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = (S)-2,3-diaminopropanoic acid
  SITE 6: note = Amidated Lysine(1,4,7,10-tetraazacyclododecane-1,4,7,10-tetraacetic acid)
AGAXXK

SEQ ID NO: 518 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = (S)-2,3-diaminopropanoic acid
  SITE 6: note = Amidated Lysine(1,4,7,10-tetraazacyclododecane-1,4,7,10-tetraacetic acid)
AGAXXK

SEQ ID NO: 519-526 - skipped (8 entries)

SEQ ID NO: 527 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = 3-(2-(2-aminoethoxy)ethoxy)propanoic acid
  SITE 6: note = Amidated Lysine(1,4,7,10-tetraazacyclododecane-1,4,7,10-tetraacetic acid)
AGAXXK

SEQ ID NO: 528 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = (S)-2,3-diaminopropanoic acid
  SITE 6: note = Amidated Lysine(1,4,7,10-tetraazacyclododecane-1,4,7,10-tetraacetic acid)
FGAXKK

SEQ ID NO: 529 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 6: note = Amidated Lysine(1,4,7,10-tetraazacyclododecane-1,4,7,10-tetraacetic acid)
FGAXKK

SEQ ID NO: 530 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = 6-aminohexanoic acid
  SITE 6: note = Amidated Lysine(1,4,7,10-tetraazacyclododecane-1,4,7,10-tetraacetic acid)
AGAXXK

SEQ ID NO: 531 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = Lys1,4,7,10-tetraazacyclododecane-1,4,7,10-tetraace tic acid
  SITE 6: note = Amidated 6-aminohexanoic acid
AGAXKX

SEQ ID NO: 532 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = Lys1,4,7,10-tetraazacyclododecane-1,4,7,10-tetraace tic acid
  SITE 6: note = Amidated 6-aminohexanoic acid
AGAXKX

SEQ ID NO: 533 - protein, 6 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lys1,4,7,10-tetraazacyclododecane-1,4,7,10-tetraace tic acid
  SITE 6: note = Amidated 6-aminohexanoic acid
FGAXKX

SEQ ID NO: 534 - protein, 6 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lys1,4,7,10-tetraazacyclododecane-1,4,7,10-tetraace tic acid
  SITE 6: note = Amidated 6-aminohexanoic acid
FGAXKX

SEQ ID NO: 535 - protein, 6 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Lys1,4,7,10-tetraazacyclododecane-1,4,7,10-tetraace tic acid
  SITE 5: note = Amidated 6-aminohexanoic acid
FGAXKX

SEQ ID NO: 536 - protein, 6 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lys(1,4,7,10-tetraazacyclododecane-1,4,7,10-tetraac etic acid)
  SITE 6: note = Amidated 2-(2-(2-aminoethoxy)ethoxy)acetic acid
FGAXKX

SEQ ID NO: 537 - protein, 6 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated Lysine
FGAXKK

SEQ ID NO: 538 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
FGAXKXK

SEQ ID NO: 539 - protein, 7 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 6: note = (S)-2-amino-3-(4-fluorobenzamido)propanoic acid
  SITE 7: note = Amidated Lysine(1,4,7,10-tetraazacyclododecane-1,4,7,10-tetraacetic acid)
FGAXAXK

SEQ ID NO: 540 - protein, 7 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = 6-aminohexanoic acid
  SITE 6: note = (S)-2-amino-3-(4-fluorobenzamido)propanoic acid
  SITE 7: note = Amidated Lysine(1,4,7,10-tetraazacyclododecane-1,4,7,10-tetraacetic acid)
FGAXXXK

SEQ ID NO: 541 - protein, 7 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = 6-aminohexanoic acid
  SITE 6: note = (S)-2-amino-3-(4-fluorobenzamido)propanoic acid
  SITE 7: note = Amidated Lysine(1,4,7,10-tetraazacyclododecane-1,4,7,10-tetraacetic acid)
FGAXXXK

SEQ ID NO: 542 - protein, 5 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-5-(4-benzylpiperazin-1-yl)pentanoic acid
  SITE 5: note = Amidated Lysine(1,4,7,10-tetraazacyclododecane-1,4,7,10-tetraacetic acid)
FGAXK

SEQ ID NO: 543-547 - skipped (5 entries)

SEQ ID NO: 548 - protein, 6 aa, synthetic construct
  SITE 1: note = (S)-cyclohexylalanine
  SITE 2: note = (S)-2-amino-2-cycloheptylacetic acid
  SITE 4: note = (S)-2-amino-2-(1-carbamimidoylpiperidin-4-yl)acetic acid
  SITE 5: note = (S)-2-amino-3-(4-fluorobenzamido)propanoic acid
  SITE 6: note = Amidated Lysine(1,4,7,10-tetraazacyclododecane-1,4,7,10-tetraacetic acid)
AGAXXK

SEQ ID NO: 549 - protein, 6 aa, synthetic construct
  SITE 1: note = (S)-cyclohexylalanine
  SITE 2: note = (S)-2-amino-2-cycloheptylacetic acid
  SITE 4: note = (S)-2-amino-2-(1-carbamimidoylpiperidin-4-yl)acetic acid
  SITE 5: note = (S)-2-amino-3-(4-fluorobenzamido)propanoic acid
  SITE 6: note = Amidated Lysine(1,4,7,10-tetraazacyclododecane-1,4,7,10-tetraacetic acid)
AGAXXK

SEQ ID NO: 550 - protein, 6 aa, synthetic construct
  SITE 1: note = (S)-2-amino-3-cycloheptylpropanoic acid
  SITE 2: note = (S)-cyclohexylglycine
  SITE 4: note = (S)-2-amino-2-(1-carbamimidoylpiperidin-4-yl)acetic acid
  SITE 5: note = (S)-2-amino-3-(4-fluorobenzamido)propanoic acid
  SITE 6: note = Amidated Lysine(1,4,7,10-tetraazacyclododecane-1,4,7,10-tetraacetic acid)
AGAXXK

SEQ ID NO: 551-552 - skipped (2 entries)

SEQ ID NO: 553 - protein, 6 aa, synthetic construct
  SITE 1: note = (S)-2-amino-3-cycloheptylpropanoic acid
  SITE 2: note = (S)-cyclohexylglycine
  SITE 4: note = (S)-2-amino-2-(1-carbamimidoylpiperidin-4-yl)acetic acid
  SITE 5: note = (S)-2-amino-3-(4-fluorobenzamido)propanoic acid
  SITE 6: note = Amidated Lysine(1,4,7,10-tetraazacyclododecane-1,4,7,10-tetraacetic acid)
AGAXXK

SEQ ID NO: 554 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 6: note = 6-aminohexanoic acid
  SITE 7: note = Amidated Lysine(1,4,7,10-tetraazacyclododecane-1,4,7,10-tetraacetic acid)
FGAXKXK

SEQ ID NO: 555 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 6: note = Amidated 6-aminohexanoic acid
AGAXKX

SEQ ID NO: 556 - protein, 6 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 6: note = Amidated 6-aminohexanoic acid
FGAXKX

SEQ ID NO: 557 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = N6-(4-fluorobenzoyl)-L-lysine
  SITE 6: note = Amidated Lysine(1,4,7,10-tetraazacyclododecane-1,4,7,10-tetraacetic acid)
AGAXKK

SEQ ID NO: 558 - protein, 6 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = N6-(4-fluorobenzoyl)-L-lysine
  SITE 6: note = Amidated Lysine(1,4,7,10-tetraazacyclododecane-1,4,7,10-tetraacetic acid)
AGAXKK

SEQ ID NO: 559 - protein, 5 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-5-(4-methylpiperazin-1-yl)pentanoic acid
  SITE 5: note = Amidated Lysine(1,4,7,10-tetraazacyclododecane-1,4,7,10-tetraacetic acid)
FGAXK

SEQ ID NO: 560 - protein, 5 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-5-(4-methylpiperazin-1-yl)pentanoic acid
  SITE 5: note = Amidated Lysine(1,4,7,10-tetraazacyclododecane-1,4,7,10-tetraacetic acid)
FGARK

SEQ ID NO: 561 - protein, 5 aa, synthetic construct
  SITE 1: note = (S)-2-amino-3-(2,4-difluorophenyl)propanoic acid
  SITE 2: note = (S)-cyclohexylglycine
  SITE 5: note = Amidated Lysine(1,4,7,10-tetraazacyclododecane-1,4,7,10-tetraacetic acid)
FGARK

SEQ ID NO: 562 - protein, 5 aa, synthetic construct
  SITE 1: note = (S)-2-amino-3-(3,4-difluorophenyl)propanoic acid
  SITE 2: note = (S)-cyclohexylglycine
  SITE 5: note = Amidated Lysine(1,4,7,10-tetraazacyclododecane-1,4,7,10-tetraacetic acid)
FGARK

SEQ ID NO: 563 - protein, 5 aa, synthetic construct
  SITE 1: note = (S)-2-amino-3-(2,4-difluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
FGAXK

SEQ ID NO: 564 - protein, 5 aa, synthetic construct
  SITE 1: note = (S)-2-amino-3-(2,4-difluorophenyl)propanoic acid
  SITE 2: note = (S)-cyclohexylglycine
  SITE 5: note = Amidated Lysine(1,4,7,10-tetraazacyclododecane-1,4,7,10-tetraacetic acid)
FGARK

SEQ ID NO: 565 - skipped

SEQ ID NO: 566 - protein, 5 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-5-(4-methylpiperazin-1-yl)pentanoic acid
  SITE 5: note = Amidated Lysine(1,4,7,10-tetraazacyclododecane-1,4,7,10-tetraacetic acid)
AGAXK

SEQ ID NO: 567 - protein, 5 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-5-(4-methylpiperazin-1-yl)pentanoic acid
  SITE 5: note = Amidated Lysine(1,4,7,10-tetraazacyclododecane-1,4,7,10-tetraacetic acid)
AGAXK

SEQ ID NO: 568 - protein, 5 aa, synthetic construct
  SITE 1: note = (S)-2-amino-3-(perfluorophenyl)propanoic acid
  SITE 2: note = (S)-cyclohexylglycine
  SITE 5: note = Amidated Lysine(1,4,7,10-tetraazacyclododecane-1,4,7,10-tetraacetic acid)
FGARK

SEQ ID NO: 569 - protein, 5 aa, synthetic construct
  SITE 1: note = (S)-cyclohexylalanine
  SITE 2: note = (S)-cyclohexylglycine
  SITE 5: note = Amidated (S)-3,7-diaminoheptanamide(1,4,7,10-tetraa zacyclododecane-1,4,7,10-tetraacetic acid)
AGARX

SEQ ID NO: 570-573 - skipped (4 entries)

SEQ ID NO: 574 - protein, 5 aa, synthetic construct
  SITE 1: note = (S)-2-([1,1'-biphenyl]-4-yl)-2-aminoacetic acid
  SITE 2: note = (S)-2-([1,1'-biphenyl]-4-yl)-2-aminoacetic acid
  SITE 5: note = Amidated Lysine(1,4,7,10-tetraazacyclododecane-1,4,7,10-tetraacetic acid)
FFARK

SEQ ID NO: 575 - protein, 5 aa, synthetic construct
  SITE 1: note = (S)-2-amino-3-(3,4,5-trifluorophenyl)propanoic acid
  SITE 2: note = (S)-cyclohexylglycine
  SITE 5: note = Amidated Lysine(1,4,7,10-tetraazacyclododecane-1,4,7,10-tetraacetic acid)
FGARK

SEQ ID NO: 576 - protein, 5 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 577 - protein, 5 aa, synthetic construct
  SITE 1: note = (S)-2-amino-3-cyclopentylpropanoic acid
  SITE 2: note = (S)-cyclohexylglycine
  SITE 5: note = Amidated Lysine(1,4,7,10-tetraazacyclododecane-1,4,7,10-tetraacetic acid)
AGARK

SEQ ID NO: 578 - protein, 6 aa, synthetic construct
  SITE 1: note = (S)-cyclohexylalanine
  SITE 2: note = (S)-cyclohexylglycine
  SITE 5: note = Amidated Lysine(1,4,7,10-tetraazacyclododecane-1,4,7,10-tetraacetic acid)
AGARYK

SEQ ID NO: 579 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 6: note = 6-aminohexanoic acid
  SITE 7: note = Amidated Lysine(1,4,7,10-tetraazacyclododecane-1,4,7,10-tetraacetic acid)
FGAXKXK

SEQ ID NO: 580 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = (S)-2-aminohexanoic acid
  SITE 6: note = 6-aminohexanoic acid
  SITE 7: note = Amidated Lysine(1,4,7,10-tetraazacyclododecane-1,4,7,10-tetraacetic acid)
FGAXXXK

SEQ ID NO: 581 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated (S)-2-aminohexanoic acid(1,4,7,10-tetraazacyclododecane-1,4,7,10-tetraacetic acid)
FGAXKXX

SEQ ID NO: 582 - protein, 7 aa, synthetic construct
  SITE 1: note = (S)-2-amino-3-(4-fluorophenyl)propanoic acid
  SITE 2: note = (S)-cyclohexylglycine
  SITE 3: note = (S)-2-aminohexanoic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated (S)-2-aminohexanoic acid(1,4,7,10-tetraazacyclododecane-1,4,7,10-tetraacetic acid)
FGXXKXK

SEQ ID NO: 583 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated 6-aminohexanoic acid
AGAXKX

SEQ ID NO: 584 - protein, 6 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated 6-aminohexanoic acid
AGAXKX

SEQ ID NO: 585 - skipped

SEQ ID NO: 586 - protein, 6 aa, synthetic construct
  SITE 1: note = (S)-2-amino-3-cycloheptylpropanoic acid
  SITE 2: note = (S)-2-amino-2-cycloheptylacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated 6-aminohexanoic acid
AGAXKX

SEQ ID NO: 587 - protein, 6 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated 6-aminohexanoic acid
AGAXKX

SEQ ID NO: 588 - protein, 6 aa, synthetic construct
  SITE 1: note = (S)-2-amino-3-cycloheptylpropanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated 6-aminohexanoic acid
AGAXKX

SEQ ID NO: 589 - protein, 7 aa, synthetic construct
  SITE 1: note = (S)-2-amino-3-cycloheptylpropanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated 6-aminohexanoic acid
AGAXKXK

SEQ ID NO: 590 - protein, 7 aa, synthetic construct
  SITE 1: note = (S)-2-amino-3-cycloheptylpropanoic acid
  SITE 2: note = (S)-2-amino-2-cycloheptylacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated 6-aminohexanoic acid
AGAXKXK

SEQ ID NO: 591 - protein, 7 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated 6-aminohexanoic acid
AGAXKXK

SEQ ID NO: 592 - protein, 7 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Lysine(1,4,7,10-tetraazacyclododecane-1,4,7,10-tetr aacetic acid)
  SITE 6: note = Amidated 6-aminohexanoic acid
AGAFKXK

SEQ ID NO: 593 - protein, 7 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
AGAFKXK

SEQ ID NO: 594 - protein, 7 aa, synthetic construct
  SITE 1: note = (S)-2-amino-3-(4-fluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
FGAFKXK

SEQ ID NO: 595 - protein, 7 aa, synthetic construct
  SITE 1: note = (S)-2-amino-3-(3,4-difluorophenyl)propanoic acid
  SITE 2: note = (S)-2-amino-2-(2,3-dihydro-1H-inden-2-yl)acetic acid
  SITE 4: note = (S)-2-amino-3-(4-aminophenyl)propanoic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
FGAFKXK

SEQ ID NO: 596 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 5: note = Amidated Lysine
FGAXKXK

SEQ ID NO: 597 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated (S)-2-aminohexanoic acid
FGAXKXX

SEQ ID NO: 598 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated (S)-2-aminohexanoic acid
FGAXKXX

SEQ ID NO: 599 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 5: note = Amidated Lysine
FGAXKXK

SEQ ID NO: 600 - protein, 7 aa, synthetic construct
  SITE 1: note = (S)-2-amino-3-cycloheptylpropanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated Lysine
AGAXKXK

SEQ ID NO: 601 - protein, 7 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated Lysine
AGAXKXK

SEQ ID NO: 602 - protein, 5 aa, synthetic construct
  SITE 2: note = (S)-2-amino-2-(2,3-dihydro-1H-inden-2-yl)acetic acid
  SITE 5: note = Amidated Lysine(1,4,7,10-tetraazacyclododecane-1,4,7,10-tetraacetic acid)
FGARK

SEQ ID NO: 603 - protein, 5 aa, synthetic construct
  SITE 1: note = (S)-2-amino-3-(2,5-dichlorophenyl)propanoic acid
  SITE 2: note = (S)-cyclohexylglycine
  SITE 5: note = Amidated Lysine(1,4,7,10-tetraazacyclododecane-1,4,7,10-tetraacetic acid)
FGARK

SEQ ID NO: 604 - protein, 5 aa, synthetic construct
  SITE 1: note = (S)-cyclohexylalanine
  SITE 2: note = (S)-cyclohexylglycine
  SITE 4: note = (S)-2-amino-4-(1H-imidazol-4-yl)butanoic acid
  SITE 5: note = Amidated Lysine(1,4,7,10-tetraazacyclododecane-1,4,7,10-tetraacetic acid)
AGAXK

SEQ ID NO: 605 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = (S)-2-amino-3-(4-(aminomethyl)phenyl)propanoic acid-1,4,7,10-tetraazacyclododecane-1,4,7,10-tetraacetic acid OR (S)-2,2',2''-(10-(2-((4-(2-amino-2-carboxyethyl)be nzyl)amino)-2-oxoethyl)-1,4,7,10-tetraazacyclododecane-1,4 ,7-triyl)triacetic acid
  SITE 6: note = 6-aminohexanoic acid
  SITE 7: note = Amidated Lysine
FGAXXXK

SEQ ID NO: 606 - protein, 7 aa, synthetic construct
  SITE 1: note = (S)-2-amino-3-(4-fluorophenyl)propanoic acid
  SITE 2: note = (S)-2-amino-2-cycloheptylacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 5: note = Amidated Lysine
FGAXKXK

SEQ ID NO: 607 - protein, 6 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidatd 2-((2-aminoethyl)(methyl)amino)acetamide
FGAXKX

SEQ ID NO: 608 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
  SITE 6: note = 2-((2-aminoethyl)(methyl)amino)acetamide
  SITE 7: note = Amidated Lysine
FGAXKXK

SEQ ID NO: 609 - protein, 7 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
  SITE 6: note = 2-((2-aminoethyl)(methyl)amino)acetamide
  SITE 7: note = Amidated (S)-2-aminohexanoic acid
FGAXKXX

SEQ ID NO: 610 - protein, 6 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
  SITE 6: note = Amidaetd 2-((2-aminoethyl)(methyl)amino)acetamide
FGAXKX

SEQ ID NO: 611 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
  SITE 6: note = 2-(2-(2-aminoethoxy)ethoxy)acetic acid
  SITE 7: note = Amidated N6-(4-(p-tolyl)butanoyl)-L-lysine
FGAXKXK

SEQ ID NO: 612 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
  SITE 6: note = 2-(2-(2-aminoethoxy)ethoxy)acetic acid
  SITE 5: note = Amidated Glutamic acid
FGAXKXE

SEQ ID NO: 613 - protein, 7 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 5: note = Amidated Lysine
FFAXKXK

SEQ ID NO: 614 - protein, 7 aa, synthetic construct
  SITE 1: note = (S)-2-amino-3-(3,4-difluorophenyl)propanoic acid
  SITE 2: note = (R)-2-amino-3-(3,4-difluorophenyl)propanoic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 5: note = Amidated Lysine
FFAXKXK

SEQ ID NO: 615 - protein, 7 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 5: note = Amidated Lysine
  SITE 6: note = 2-(2-(2-aminoethoxy)ethoxy)acetic acid
  SITE 7: note = Amidated N6-(4-(p-tolyl)butanoyl)-L-lysine
FFAXKXK

SEQ ID NO: 616 - protein, 7 aa, synthetic construct
  SITE 1: note = (S)-2-amino-3-cycloheptylpropanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
AFAXKXK

SEQ ID NO: 617 - protein, 7 aa, synthetic construct
  SITE 1: note = (S)-2-amino-3-cycloheptylpropanoic acid
  SITE 2: note = (S)-2-([1,1'-biphenyl]-4-yl)-2-aminoacetic acid
  SITE 5: note = Lysine(1,4,7,10-tetraazacyclododecane-1,4,7,10-tetr aacetic acid)
  SITE 4: note = (S)-2-amino-2-(1-carbamimidoylpiperidin-4-yl)acetic acid
  SITE 6: note = 2-(2-(2-aminoethoxy)ethoxy)acetic acid
  SITE 7: note = Amidated Lysine
AFAXKXK

SEQ ID NO: 618 - protein, 7 aa, synthetic construct
  SITE 1: note = (S)-2-amino-3-(4-fluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
FFAXKXK

SEQ ID NO: 619 - protein, 7 aa, synthetic construct
  SITE 1: note = (S)-2-amino-3-cycloheptylpropanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
AFAXKXK

SEQ ID NO: 620 - protein, 7 aa, synthetic construct
  SITE 1: note = (S)-2-amino-3-cycloheptylpropanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
AFAXKXK

SEQ ID NO: 621 - protein, 7 aa, synthetic construct
  SITE 1: note = (S)-2-amino-3-(4-fluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
FFAXKXK

SEQ ID NO: 622 - protein, 7 aa, synthetic construct
  SITE 1: note = (S)-2-amino-3-(4-fluorophenyl)propanoic acid
  SITE 2: note = (S)-2-([1,1'-biphenyl]-4-yl)-2-aminoacetic acid
  SITE 3: note = (S)-2-aminohexanoic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
FFXXKXK

SEQ ID NO: 623 - protein, 7 aa, synthetic construct
  SITE 1: note = (S)-2-amino-3-(4-fluorophenyl)propanoic acid
  SITE 2: note = (S)-2-([1,1'-biphenyl]-4-yl)-2-aminoacetic acid
  SITE 3: note = (S)-2-aminohexanoic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
FFXXKXK

SEQ ID NO: 624 - protein, 5 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AFAXK

SEQ ID NO: 625 - protein, 5 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AFAXK

SEQ ID NO: 626 - protein, 5 aa, synthetic construct
  SITE 1: note = (S)-2-amino-3-cyclopentylpropanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AFAXK

SEQ ID NO: 627 - protein, 5 aa, synthetic construct
  SITE 1: note = (S)-2-amino-3-cyclopentylpropanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AFAXK

SEQ ID NO: 628 - protein, 5 aa, synthetic construct
  SITE 1: note = (S)-2-amino-3-cyclopentylpropanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AFAXK

SEQ ID NO: 629 - protein, 5 aa, synthetic construct
  SITE 1: note = (S)-2-amino-3-cyclopentylpropanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
AFAXK

SEQ ID NO: 630 - protein, 6 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-7-hydroxy-1,2,3,4-tetrahydroisoquinoline-3-carb oxylic acid
  SITE 6: note = Amidated Lysine(1,4,7,10-tetraazacyclododecane-1,4,7,10-tetraacetic acid)
AFAXXK

SEQ ID NO: 631 - protein, 6 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-piperidine-2-carboxylic acid
  SITE 6: note = Amidated Lysine(1,4,7,10-tetraazacyclododecane-1,4,7,10-tetraacetic acid)
AFAXXK

SEQ ID NO: 632 - protein, 6 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-piperidine-2-carboxylic acid
  SITE 6: note = Amidated Lysine(1,4,7,10-tetraazacyclododecane-1,4,7,10-tetraacetic acid)
FFAXXK

SEQ ID NO: 633 - protein, 6 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-7-hydroxy-1,2,3,4-tetrahydroisoquinoline-3-carb oxylic acid
  SITE 6: note = Amidated Lysine(1,4,7,10-tetraazacyclododecane-1,4,7,10-tetraacetic acid)
AFAXXK

SEQ ID NO: 634 - protein, 6 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-7-hydroxy-1,2,3,4-tetrahydroisoquinoline-3-carb oxylic acid
  SITE 6: note = Amidated Lysine(1,4,7,10-tetraazacyclododecane-1,4,7,10-tetraacetic acid)
AFAXXK

SEQ ID NO: 635 - protein, 6 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-piperidine-2-carboxylic acid
  SITE 6: note = Amidated Lysine(1,4,7,10-tetraazacyclododecane-1,4,7,10-tetraacetic acid)
FFAXXK

SEQ ID NO: 636 - protein, 6 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-7-hydroxy-1,2,3,4-tetrahydroisoquinoline-3-carb oxylic acid
  SITE 6: note = Amidated Lysine(1,4,7,10-tetraazacyclododecane-1,4,7,10-tetraacetic acid)
FFAXXK

SEQ ID NO: 637 - protein, 8 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-7-hydroxy-1,2,3,4-tetrahydroisoquinoline-3-carb oxylic acid
  SITE 6: note = Lysine(1,4,7,10-tetraazacyclododecane-1,4,7,10-tetr aacetic acid)
  SITE 7: note = 6-aminohexanoic acid
  SITE 8: note = Amidated Lysine
FFAXXKXK

SEQ ID NO: 638 - protein, 8 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-7-hydroxy-1,2,3,4-tetrahydroisoquinoline-3-carb oxylic acid
  SITE 6: note = Lysine(1,4,7,10-tetraazacyclododecane-1,4,7,10-tetr aacetic acid)
  SITE 7: note = 6-aminohexanoic acid
  SITE 8: note = Amidated Lysine
FFAXXKXK

SEQ ID NO: 639 - protein, 6 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-7-hydroxy-1,2,3,4-tetrahydroisoquinoline-3-carb oxylic acid
  SITE 6: note = Amidated Lysine(1,4,7,10-tetraazacyclododecane-1,4,7,10-tetraacetic acid)
FFAXXK

SEQ ID NO: 640 - protein, 8 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-7-hydroxy-1,2,3,4-tetrahydroisoquinoline-3-carb oxylic acid
  SITE 6: note = Lysine(1,4,7,10-tetraazacyclododecane-1,4,7,10-tetr aacetic acid)
  SITE 7: note = 6-aminohexanoic acid
  SITE 8: note = Amidated Lysine
FFAXXKXK

SEQ ID NO: 641 - protein, 8 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-7-hydroxy-1,2,3,4-tetrahydroisoquinoline-3-carb oxylic acid
  SITE 6: note = Lysine(1,4,7,10-tetraazacyclododecane-1,4,7,10-tetr aacetic acid)
  SITE 7: note = 6-aminohexanoic acid
  SITE 8: note = Amidated Lysine
FFAXXKXK

SEQ ID NO: 642 - protein, 8 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-piperidine-2-carboxylic acid
  SITE 6: note = Lysine(1,4,7,10-tetraazacyclododecane-1,4,7,10-tetr aacetic acid)
  SITE 7: note = 6-aminohexanoic acid
  SITE 8: note = Amidated Lysine
AFAXXKXK

SEQ ID NO: 643 - protein, 8 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-piperidine-2-carboxylic acid
  SITE 6: note = Amidated Lysine(1,4,7,10-tetraazacyclododecane-1,4,7,10-tetraacetic acid)
  SITE 7: note = 6-aminohexanoic acid
  SITE 8: note = Amidated Lysine
AFAXXKXK

SEQ ID NO: 644 - protein, 8 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-piperidine-2-carboxylic acid
  SITE 6: note = Amidated Lysine(1,4,7,10-tetraazacyclododecane-1,4,7,10-tetraacetic acid)
  SITE 7: note = 6-aminohexanoic acid
  SITE 8: note = Amidated Glutamic acid
AFAXXKXE

SEQ ID NO: 645 - protein, 8 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-piperidine-2-carboxylic acid
  SITE 6: note = Lysine(1,4,7,10-tetraazacyclododecane-1,4,7,10-tetr aacetic acid)
  SITE 7: note = 6-aminohexanoic acid
  SITE 8: note = Amidated Lysine
AFAXXKXK

SEQ ID NO: 646 - protein, 8 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-piperidine-2-carboxylic acid
  SITE 6: note = Amidated Lysine(1,4,7,10-tetraazacyclododecane-1,4,7,10-tetraacetic acid)
  SITE 7: note = 6-aminohexanoic acid
  SITE 8: note = Amidated Lysine
AFAXXKXK

SEQ ID NO: 647 - protein, 8 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-piperidine-2-carboxylic acid
  SITE 5: note = Lysine(1,4,7,10-tetraazacyclododecane-1,4,7,10-tetr aacetic acid)
  SITE 7: note = 6-aminohexanoic acid
  SITE 5: note = Amidated Glutamic acid
AFAXXKXE

SEQ ID NO: 648 - protein, 6 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 6: note = Amidated Lysine(1,4,7,10-tetraazacyclododecane-1,4,7,10-tetraacetic acid)
FFAXPK

SEQ ID NO: 649 - protein, 6 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = hydroxy-Proline
  SITE 6: note = Amidated Lysine(1,4,7,10-tetraazacyclododecane-1,4,7,10-tetraacetic acid)
FFAXPK

SEQ ID NO: 650 - protein, 6 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-isoindoline-1-carboxylic acid
  SITE 6: note = Amidated Lysine(1,4,7,10-tetraazacyclododecane-1,4,7,10-tetraacetic acid)
FFAXXK

SEQ ID NO: 651 - protein, 6 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 6: note = Amidated Lysine(1,4,7,10-tetraazacyclododecane-1,4,7,10-tetraacetic acid)
AFAXPK

SEQ ID NO: 652 - protein, 6 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = hydroxy-Proline
  SITE 6: note = Amidated Lysine(1,4,7,10-tetraazacyclododecane-1,4,7,10-tetraacetic acid)
FFAXKK

SEQ ID NO: 653 - protein, 6 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(1-carbamimidoylpiperidin-4-yl)acetic acid
  SITE 5: note = (S)-isoindoline-1-carboxylic acid
  SITE 6: note = Amidated Lysine(1,4,7,10-tetraazacyclododecane-1,4,7,10-tetraacetic acid)
FFAXXK

SEQ ID NO: 654 - protein, 6 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated Lysine
FGAXKK

SEQ ID NO: 655 - protein, 5 aa, synthetic construct
  SITE 1: note = (S)-2-amino-3-(3,4,5-trifluorophenyl)propanoic acid
  SITE 2: note = (S)-cyclohexylglycine
  SITE 3: note = Lysine(1,4,7,10-tetraazacyclododecane-1,4,7,10-tetr aacetic acid)
  SITE 4: note = (S)-2-amino-2-(1-carbamimidoylpiperidin-4-yl)acetic acid
  SITE 5: note = Amidated Lysine
FGKXK

SEQ ID NO: 656 - protein, 7 aa, synthetic construct
  SITE 1: note = (S)-2-amino-3-(3,4,5-trifluorophenyl)propanoic acid
  SITE 2: note = (S)-cyclohexylglycine
  SITE 3: note = (S)-2-aminohexanoic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
FGXXKXK

SEQ ID NO: 657 - protein, 5 aa, synthetic construct
  SITE 1: note = (S)-2-([1,1'-biphenyl]-4-yl)-2-aminoacetic acid
  SITE 2: note = (S)-cyclohexylglycine
  SITE 3: note = Lysine(1,4,7,10-tetraazacyclododecane-1,4,7,10-tetr aacetic acid)
  SITE 4: note = (S)-2-amino-2-(1-carbamimidoylpiperidin-4-yl)acetic acid
  SITE 5: note = Amidated Lysine
FGKXK

SEQ ID NO: 658 - protein, 6 aa, synthetic construct
  SITE 1: note = (S)-2-([1,1'-biphenyl]-4-yl)-2-aminoacetic acid
  SITE 2: note = (S)-cyclohexylglycine
  SITE 3: note = Amidated Lysine(1,4,7,10-tetraazacyclododecane-1,4,7,10-tetraacetic acid)
  SITE 4: note = (S)-2-amino-2-(1-carbamimidoylpiperidin-4-yl)acetic acid
  SITE 6: note = Amidated 3-(2-(2-aminoethoxy)ethoxy)propanoic acid
FGKXKX

SEQ ID NO: 659 - protein, 6 aa, synthetic construct
  SITE 1: note = (S)-2-([1,1'-biphenyl]-4-yl)-2-aminoacetic acid
  SITE 2: note = (S)-cyclohexylglycine
  SITE 3: note = Lysine(1,4,7,10-tetraazacyclododecane-1,4,7,10-tetr aacetic acid)
  SITE 4: note = (S)-2-amino-2-(1-carbamimidoylpiperidin-4-yl)acetic acid
  SITE 6: note = Amidated 3-(2-(2-aminoethoxy)ethoxy)propanoic acid
FGKXKX

SEQ ID NO: 660 - protein, 7 aa, synthetic construct
  SITE 1: note = (S)-2-amino-3-cyclopentylpropanoic acid
  SITE 2: note = (S)-cyclohexylglycine
  SITE 3: note = (S)-2-aminohexanoic acid
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 6-aminohexanoic acid
  SITE 7: note = Amidated Lysine
AGXXKXK

SEQ ID NO: 661-662 - skipped (2 entries)

SEQ ID NO: 663 - protein, 5 aa, synthetic construct
  SITE 1: note = (S)-2-amino-3-(4-fluorophenyl)propanoic acid
  SITE 2: note = (S)-2-amino-2-cycloheptylacetic acid
  SITE 5: note = Amidated Lysine(1,4,7,10-tetraazacyclododecane-1,4,7,10-tetraacetic acid)
FGAXK

SEQ ID NO: 664 - skipped

SEQ ID NO: 665 - protein, 6 aa, synthetic construct
  SITE 1: note = (S)-2-([1,1'-biphenyl]-4-yl)-2-aminoacetic acid
  SITE 2: note = (S)-2-([1,1'-biphenyl]-4-yl)-2-aminoacetic acid
  SITE 3: note = Lysine(1,4,7,10-tetraazacyclododecane-1,4,7,10-tetr aacetic acid)
  SITE 4: note = (S)-2-amino-2-(1-carbamimidoylpiperidin-4-yl)acetic acid
  SITE 6: note = Amidated 3-(2-(2-aminoethoxy)ethoxy)propanoic acid
FGKXKX

SEQ ID NO: 666 - protein, 5 aa, synthetic construct
  SITE 1: note = (S)-cyclohexylalanine
  SITE 2: note = (S)-2-([1,1'-biphenyl]-4-yl)-2-aminoacetic acid
  SITE 5: note = Amidated Lysine(1,4,7,10-tetraazacyclododecane-1,4,7,10-tetraacetic acid)
AFARK

SEQ ID NO: 667 - protein, 5 aa, synthetic construct
  SITE 1: note = (S)-cyclohexylalanine
  SITE 2: note = (S)-2-([1,1'-biphenyl]-4-yl)-2-aminoacetic acid
  SITE 4: note = (S)-2-amino-2-(piperidin-4-yl)acetic acid
  SITE 5: note = Amidated Lysine(1,4,7,10-tetraazacyclododecane-1,4,7,10-tetraacetic acid)
AFAGK

SEQ ID NO: 668 - protein, 6 aa, synthetic construct
  SITE 1: note = (S)-cyclohexylalanine
  SITE 2: note = (S)-2-amino-2-(2,3-dihydro-1H-inden-2-yl)acetic acid
  SITE 4: note = (S)-2-amino-2-(1-carbamimidoylpiperidin-4-yl)acetic acid
  SITE 5: note = N6-(4-fluorobenzoyl)-L-lysine
  SITE 6: note = Amidated Lysine(1,4,7,10-tetraazacyclododecane-1,4,7,10-tetraacetic acid)
AGAXKK

SEQ ID NO: 669 - protein, 7 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-3-(6-aminopyridin-3-yl)propanoic acid
  SITE 6: note = 2-(2-(2-aminoethoxy)ethoxy)acetic acid
  SITE 7: note = Amidated Lysine(1,4,7,10-tetraazacyclododecane-1,4,7,10-tetraacetic acid)
AGAXKXK

SEQ ID NO: 670 - protein, 7 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-3-(6-aminopyridin-3-yl)propanoic acid
  SITE 6: note = 2-(2-(2-aminoethoxy)ethoxy)acetic acid
  SITE 7: note = Amidated Lysine(1,4,7,10-tetraazacyclododecane-1,4,7,10-tetraacetic acid)
AGAXKXK

SEQ ID NO: 671 - protein, 7 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-3-(6-aminopyridin-3-yl)propanoic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated Lysine
AGAXKXK

SEQ ID NO: 672 - protein, 7 aa, synthetic construct
  SITE 1: note = (S)-2-amino-3-cycloheptylpropanoic acid
  SITE 2: note = (S)-2-amino-2-(2,3-dihydro-1H-inden-2-yl)acetic acid
  SITE 4: note = (S)-2-amino-3-(6-aminopyridin-3-yl)propanoic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated Lysine
AGAXKXK

SEQ ID NO: 673 - protein, 5 aa, synthetic construct
  SITE 1: note = (S)-cyclohexylalanine
  SITE 2: note = (S)-cyclohexylglycine
  SITE 4: note = (S)-2-amino-3-(4-((R)-2,6-dioxohexahydropyrimidine- 4-carboxamido)phenyl)propanoic acid
  SITE 5: note = Amidated Lysine(1,4,7,10-tetraazacyclododecane-1,4,7,10-tetraacetic acid)
AGAXK

SEQ ID NO: 674 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = Amidated 6-aminohexanoic acid
AGAXKX

SEQ ID NO: 675 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
AGAXKX

SEQ ID NO: 676 - protein, 5 aa, synthetic construct
  SITE 1: note = (S)-2-amino-3-(3,4,5-trifluorophenyl)propanoic acid
  SITE 2: note = (S)-cyclohexylglycine
  SITE 4: note = (S)-2-amino-2-(piperidin-4-yl)acetic acid
  SITE 5: note = Amidated Lysine(1,4,7,10-tetraazacyclododecane-1,4,7,10-tetraacetic acid)
FGAGK

SEQ ID NO: 677 - protein, 5 aa, synthetic construct
  SITE 1: note = (S)-2-amino-3-(3,4,5-trifluorophenyl)propanoic acid
  SITE 2: note = (S)-cyclohexylglycine
  SITE 4: note = (S)-2-amino-2-(piperidin-4-yl)acetic acid
  SITE 5: note = Amidated Lysine(1,4,7,10-tetraazacyclododecane-1,4,7,10-tetraacetic acid)
FGAGK

SEQ ID NO: 678 - protein, 5 aa, synthetic construct
  SITE 1: note = (S)-2-amino-3-(3,4,5-trifluorophenyl)propanoic acid
  SITE 2: note = (S)-cyclohexylglycine
  SITE 4: note = (S)-2-amino-2-(piperidin-4-yl)acetic acid
  SITE 5: note = Amidated Lysine(1,4,7,10-tetraazacyclododecane-1,4,7,10-tetraacetic acid)
AGAGK

SEQ ID NO: 679 - protein, 5 aa, synthetic construct
  SITE 1: note = (S)-2-amino-3-cyclopentylpropanoic acid
  SITE 2: note = (S)-cyclohexylglycine
  SITE 4: note = (S)-2-amino-2-(piperidin-4-yl)acetic acid
  SITE 5: note = Amidated Lysine(1,4,7,10-tetraazacyclododecane-1,4,7,10-tetraacetic acid)
AGAGK

SEQ ID NO: 680 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FGAGKXK

SEQ ID NO: 681 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FGAGKXK

SEQ ID NO: 682 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = Amidated 2-(2-(2-aminoethoxy)ethoxy)acetic acid
FGAGKXK

SEQ ID NO: 683 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated Glutamic acid
FGAXKXE

SEQ ID NO: 684 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated Glutamic acid
FGAGKXE

SEQ ID NO: 685 - protein, 6 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
FGAGKX

SEQ ID NO: 686 - protein, 6 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
FGAGKX

SEQ ID NO: 687 - protein, 6 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
FGAXKX

SEQ ID NO: 688 - protein, 7 aa, synthetic construct
  SITE 1: note = (S)-2-amino-3-(3,4-difluorophenyl)propanoic acid
  SITE 2: note = (S)-2-([1,1'-biphenyl]-4-yl)-2-aminoacetic acid
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FFAGKXK

SEQ ID NO: 689 - protein, 7 aa, synthetic construct
  SITE 1: note = (S)-2-amino-3-cycloheptylpropanoic acid
  SITE 2: note = (S)-2-([1,1'-biphenyl]-4-yl)-2-aminoacetic acid
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
AFAGKXK

SEQ ID NO: 690 - protein, 6 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(1-carbamimidoylpiperidin-4-yl)acetic acid
  SITE 5: note = Amidated Lysine(1,4,7,10-tetraazacyclododecane-1,4,7,10-tetraacetic acid)
  SITE 6: note = N-(2-aminoethyl)-N-methylglycine
FGAXKG

SEQ ID NO: 691-693 - skipped (3 entries)

SEQ ID NO: 694 - protein, 5 aa, synthetic construct
  SITE 1: note = (S)-2-amino-3-(4-fluorophenyl)propanoic acid
  SITE 2: note = (S)-cyclohexylglycine
  SITE 2: note = (S)-2-amino-2-(piperidin-4-yl)acetic acid
  SITE 5: note = Lysine(gallium-1,4,7,10-tetraazacyclododecane-1,4,7 ,10-tetraacetic acid)
FGAGK

SEQ ID NO: 695 - protein, 5 aa, synthetic construct
  SITE 1: note = (S)-2-amino-3-(4-fluorophenyl)propanoic acid
  SITE 2: note = (S)-cyclohexylglycine
  SITE 2: note = (S)-2-amino-2-(piperidin-4-yl)acetic acid
  SITE 5: note = Lysine(Lutetium-1,4,7,10-tetraazacyclododecane-1,4, 7,10-tetraacetic acid)
FGAGK

SEQ ID NO: 696 - protein, 5 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(piperidin-4-yl)acetic acid
  SITE 5: note = Amidated Lysine(indium-1,4,7,10-tetraazacyclododeca ne-1,4,7,10-tetraacetic acid)
FGAGK

SEQ ID NO: 697 - protein, 6 aa, synthetic construct
  SITE 1: note = (S)-cyclohexylalanine
  SITE 2: note = (S)-cyclohexylglycine
  SITE 4: note = (S)-2-amino-2-(1-carbamimidoylpiperidin-4-yl)acetic acid
  SITE 5: note = Lysine(indium(III) (R)-2,2',2''-(10-(2-((5,6-diamin o-6-oxohexyl)amino)-2-oxoethyl)-1,4,7,10-tetraazacyclodode cane-1,4,7-triyl)triacetate)
  SITE 6: note = Amidated 6-aminohexanoic acid
AGAXKX

SEQ ID NO: 698 - protein, 5 aa, synthetic construct
  SITE 1: note = (S)-2-([1,1'-biphenyl]-4-yl)-2-aminoacetic acid
  SITE 2: note = (S)-2-([1,1'-biphenyl]-4-yl)-2-aminoacetic acid
  SITE 5: note = Amidated Lysine(1,4,7,10-tetraazacyclododecane-1,4,7,10-tetraacetic acid)
FGARK

SEQ ID NO: 699 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(Lutetium-1,4,7,10-tetraazacyclododecane-1,4, 7,10-tetraacetic acid)
  SITE 6: note = 2-(2-(2-aminoethoxy)ethoxy)acetic acid
  SITE 7: note = Amidated N6-(4-(p-tolyl)butanoyl)-L-lysine
FGAGKXK

SEQ ID NO: 700 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Amidated Lysine(indium-1,4,7,10-tetraazacyclododeca ne-1,4,7,10-tetraacetic acid)
  SITE 6: note = 2-(2-(2-aminoethoxy)ethoxy)acetic acid
  SITE 7: note = Amidated N6-(4-(p-tolyl)butanoyl)-L-lysine
FGAGKXK

SEQ ID NO: 701 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(gallium-1,4,7,10-tetraazacyclododecane-1,4,7 ,10-tetraacetic acid)
  SITE 6: note = 2-(2-(2-aminoethoxy)ethoxy)acetic acid
  SITE 7: note = Amidated N6-(4-(p-tolyl)butanoyl)-L-lysine
FGAGKXK

SEQ ID NO: 702 - protein, 4 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
FGAG

SEQ ID NO: 703 - protein, 4 aa, synthetic construct
  SITE 1: note = (S)-2-amino-3-(4-fluorophenyl)propanoic acid
  SITE 2: note = (S)-cyclohexylglycine
  SITE 4: note = (S)-2-amino-2-(piperidin-4-yl)acetic acid
FGAG

SEQ ID NO: 704 - protein, 4 aa, synthetic construct
  SITE 1: note = (S)-cyclohexylalanine
  SITE 2: note = (S)-cyclohexylglycine
  SITE 4: note = (S)-2-amino-2-(piperidin-4-yl)acetic acid
AGAG

SEQ ID NO: 705 - protein, 4 aa, synthetic construct
  SITE 1: note = (S)-cyclohexylalanine
  SITE 2: note = (S)-cyclohexylglycine
AGAR

SEQ ID NO: 706 - protein, 5 aa, synthetic construct
  SITE 1: note = (S)-cyclohexylalanine
  SITE 2: note = (S)-cyclohexylglycine
  SITE 5: note = Amidated Leucine
AGARL

SEQ ID NO: 707 - protein, 4 aa, synthetic construct
  SITE 1: note = (S)-cyclohexylalanine
  SITE 2: note = (S)-cyclohexylglycine
  SITE 4: note = Amidated Arginine
AGGR

SEQ ID NO: 708 - protein, 4 aa, synthetic construct
  SITE 1: note = (S)-cyclohexylalanine
  SITE 4: note = Amidated Arginine
AAGR

SEQ ID NO: 709 - protein, 4 aa, synthetic construct
  SITE 1: note = (S)-cyclohexylalanine
  SITE 2: note = (S)-cyclohexylglycine
  SITE 4: note = Amidated Arginine
AGAR

SEQ ID NO: 710 - protein, 5 aa, synthetic construct
  SITE 1: note = (S)-cyclohexylalanine
  SITE 2: note = (S)-cyclohexylglycine
  SITE 5: note = Amidated Lysine
AGARK

SEQ ID NO: 711 - protein, 6 aa, synthetic construct
  SITE 1: note = (S)-cyclohexylalanine
  SITE 2: note = (S)-cyclohexylglycine
  SITE 6: note = Amidated Lysine
AGARLK

SEQ ID NO: 712 - protein, 6 aa, synthetic construct
  SITE 1: note = (S)-cyclohexylalanine
  SITE 2: note = (S)-cyclohexylglycine
  SITE 6: note = Amidated Lysine
AGARAK

SEQ ID NO: 713 - protein, 5 aa, synthetic construct
  SITE 1: note = (S)-cyclohexylalanine
  SITE 2: note = (S)-cyclohexylglycine
  SITE 5: note = Amidated N6-acetyllysine
AGARK

SEQ ID NO: 714 - protein, 5 aa, synthetic construct
  SITE 1: note = (S)-cyclohexylalanine
  SITE 2: note = (S)-cyclohexylglycine
  SITE 5: note = Amidated N6-(4-fluorobenzoyl)-L-lysine
AGARK

SEQ ID NO: 715 - protein, 5 aa, synthetic construct
  SITE 1: note = (S)-cyclohexylalanine
  SITE 2: note = (S)-cyclohexylglycine
  SITE 5: note = Amidated (S)-2,3-diaminopropanoic acid
AGARX

SEQ ID NO: 716 - protein, 5 aa, synthetic construct
  SITE 1: note = (S)-cyclohexylalanine
  SITE 2: note = (S)-cyclohexylglycine
  SITE 5: note = Amidated (S)-2-amino-3-(4-fluorobenzamido)propanoic acid
AGARX

SEQ ID NO: 717 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FGAXKXK

SEQ ID NO: 718 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FGAXKXK

SEQ ID NO: 719 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FGAXKXK

SEQ ID NO: 720 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FGAXKXK

SEQ ID NO: 721 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = N6-(4-(p-tolyl)butanoyl)-L-lysine
FGAXKXK

SEQ ID NO: 722 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FGAXKXK

SEQ ID NO: 723 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FGAXKXK

SEQ ID NO: 724 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 5: note = Lysine(1,4,7,10-tetraazacyclododecane-1,4,7,10-tetr aacetic acid)
  SITE 6: note = 2-(2-(2-aminoethoxy)ethoxy)acetic acid
  SITE 7: note = Amidated N6-(4-(p-tolyl)butanoyl)-L-lysine
FGAXKXK

SEQ ID NO: 725 - protein, 6 aa, Homo sapiens
SLIGKV

SEQ ID NO: 726 - protein, 6 aa, unidentified
  source 1..6: note = rodent
SLIGRL

SEQ ID NO: 727 - protein, 7 aa, synthetic construct
  SITE 1: note = (S)-2-amino-3-(3,4-difluorophenyl)propanoic acid
  SITE 2: note = (S)-cyclohexylglycine
  SITE 4: note = (S)-2-amino-2-(piperidin-4-yl)acetic acid
  SITE 6: note = 2-(2-(2-aminoethoxy)ethoxy)acetic acid
  SITE 7: note = Amidated N6-(4-(p-tolyl)butanoyl)-L-lysine
FGAXKXK